(19) United States
(12) Patent Application Publication
(10) Pub. No.: US 2025/0352638 A1
(43) Pub. Date: Nov. 20, 2025

(54) CORONAVIRUS IMMUNOGEN COMPOSITIONS AND THEIR USES

(71) Applicant: Flagship Pioneering Innovations VI, LLC, Cambridge, MA (US)

(72) Inventors: Avak KAHVEJIAN, Lexington, MA (US); Alexandra Sophie DE BOER, Somerville, MA (US); Yann Paul Guy Régis ECHELARD, Jamaica Plain, MA (US); Jennifer A. NELSON, Brookline, MA (US); Soohyun LEE, Belmont, MA (US); Michael Donato MELFI, Somerville, MA (US); Mohammad Mubeen MOSAHEB, Watertown, MA (US); Alla Alexeevna SIGOVA, Newton, MA (US)

(21) Appl. No.: 18/712,356

(22) PCT Filed: Nov. 23, 2022
(86) PCT No.: PCT/US2022/050893

§ 371 Date: May 22, 2024

Related U.S. Application Data
(60) Provisional application No. 63/283,146, filed on Nov. 24, 2021.

(51) Int. Cl. A61K 39/215; A61K 39/00; A61P 31/14; C07K 14/005

(52) U.S. Cl. CPC A61K 39/215; A61P 31/14; C07K 14/005; A61K 2039/53; A61K 2039/55516; A61K 2039/572; A61K 2039/70; C12N 2760/16122; C12N 2760/16134; C12N 2770/20022; C12N 2770/20034

(57) ABSTRACT

The disclosure provides compositions and methods comprising circular polyribonucleotides comprising a sequence encoding a coronavirus immunogen, and compositions and methods comprising linear polyribonucleotides comprising a sequence encoding one or more coronavirus immunogens. Compositions and methods are provided that are related to generating polyclonal antibodies, for example, using the disclosed circular polyribonucleotides or the disclosed linear polyribonucleotides.

### SEQUENCE LISTING

The instant application contains a Sequence Listing which has been submitted electronically in XML format and is hereby incorporated by reference in its entirety. Said XML copy, created on Nov. 26, 2024, is named “51509-063002_Sequence_Listing_11_26_24.xml” and is 833,590 bytes in size.

### BACKGROUND

COVID-19, a respiratory disease in humans caused by an infection of SARS-CoV-2, emerged in Wuhan, China, and spread worldwide, leading to the World Health Organization declaring a pandemic on Mar. 11, 2020, and resulting in millions of deaths worldwide. Therefore, there is an urgent need for vaccines and therapeutics that are active against coronaviruses and uses thereof.

### SUMMARY

The disclosure generally relates to circular polyribonucleotides comprising a sequence encoding a coronavirus immunogen and to immunogenic compositions comprising the circular polyribonucleotide. This disclosure further relates to methods of using circular polyribonucleotides comprising a sequence encoding a coronavirus immunogen and the immunogenic composition. In some embodiments, the circular polyribonucleotides and immunogenic compositions of this disclosure are used in methods of generating polyclonal antibodies. The produced polyclonal antibodies can be used in methods of prophylaxis in subjects (e.g., human subjects) or methods of treatment for subjects (e.g., human subjects) having a coronavirus infection. The produced polyclonal antibodies can be administered to subjects at high risk for exposure to coronavirus infection.

In a first aspect, the disclosure provides a circular polyribonucleotide including an open reading frame encoding a coronavirus immunogen, wherein the coronavirus immunogen includes an amino acid sequence having at least 85% sequence identity (e.g., at least 85%, 86%, 87%, 88%, 89%, 90%, 91%, 92%, 93%, 94%, 95%, 96%, 97%, 98%, 99% or 100% sequence identity) with the amino acid sequence of any one of SEQ ID NOs: 63-111 and 283-291. In some embodiments, the coronavirus immunogen includes an amino acid sequence having at least 90% (e.g., at least 90%, 91%, 92%, 93%, 94%, 95%, 96%, 97%, 98%, 99% or 100% sequence identity) sequence identity with the amino acid sequence of any one of SEQ ID NOs: 63-111 and 283-291. In some embodiments, the coronavirus immunogen includes an amino acid sequence having at least 95% (e.g., at least 95%, 96%, 97%, 98%, 99% or 100% sequence identity) sequence identity with the amino acid sequence of any one of SEQ ID NOs: 63-111 and 283-291. In some embodiments, the coronavirus immunogen includes an amino acid sequence of any one of SEQ ID NOs: 63-111 and 283-291.

In certain embodiments, the coronavirus immunogen is a RBD immunogen having at least 85% (e.g., at least 85%, 86%, 87%, 88%, 89%, 90%, 91%, 92%, 93%, 94%, 95%, 96%, 97%, 98%, 99% or 100% sequence identity) identity with the amino acid sequence of any one of SEQ ID NOs: 63-68, 74, 79, 81-86, and 98-111. In some embodiments, the coronavirus immunogen is a RBD immunogen having at least 90% (e.g., at least 90%, 91%, 92%, 93%, 94%, 95%, 96%, 97%, 98%, 99% or 100% sequence identity) identity with the amino acid sequence of any one of SEQ ID NOs: 63-68, 74, 79, 81-86, and 98-111. In some embodiments, the coronavirus immunogen is a RBD immunogen having at least 95% (e.g., at least 95%, 96%, 97%, 98%, 99% or 100% sequence identity) identity with the amino acid sequence of any one of SEQ ID NOs: 63-68, 74, 79, 81-86, and 98-111. In some embodiments, the coronavirus immunogen is a RBD immunogen having an amino acid sequence of any one of SEQ ID NOs: 63-68, 74, 79, 81-86, and 98-111.

In certain embodiments, the coronavirus immunogen is a Spike immunogen having at least 85% (e.g., at least 85%, 86%, 87%, 88%, 89%, 90%, 91%, 92%, 93%, 94%, 95%, 96%, 97%, 98%, 99% or 100% sequence identity) identity with the amino acid sequence of any one of SEQ ID NOs: 69-73, 75-78, 80, 87-97, and 283-286. In some embodiments, the coronavirus immunogen is a Spike immunogen having at least 90% (e.g., at least 90%, 91%, 92%, 93%, 94%, 95%, 96%, 97%, 98%, 99% or 100% sequence identity) identity with the amino acid sequence of any one of SEQ ID NOs: 69-73, 75-78, 80, 87-97, and 283-286. In some embodiments, the coronavirus immunogen is a Spike immunogen having at least 95% (e.g., at least 95%, 96%, 97%, 98%, 99% or 100% sequence identity) identity with the amino acid sequence of any one of SEQ ID NOs: 69-73, 75-78, 80, 87-97, and 283-286. In some embodiments, the coronavirus immunogen is a Spike immunogen having an amino acid sequence of any one of SEQ ID NOs: 69-73, 75-78, 80, 87-97, and 283-286.

In certain embodiments, the coronavirus immunogen is a nonstructural protein (nsp) having at least 85% (e.g., at least 85%, 86%, 87%, 88%, 89%, 90%, 91%, 92%, 93%, 94%, 95%, 96%, 97%, 98%, 99% or 100% sequence identity) identity with the amino acid sequence of any one of SEQ ID NOs: 291-295. In some embodiments, the coronavirus immunogen is a nsp immunogen having at least 90% (e.g., at least 90%, 91%, 92%, 93%, 94%, 95%, 96%, 97%, 98%, 99% or 100% sequence identity) identity with the amino acid sequence of any one of SEQ ID NOs: 291-295. In some embodiments, the coronavirus immunogen is a nsp immunogen having at least 95% (e.g., at least 95%, 96%, 97%, 98%, 99% or 100% sequence identity) identity with the amino acid sequence of any one of SEQ ID NOs: 291-295. In some embodiments, the coronavirus immunogen is a nsp immunogen having an amino acid sequence of any one of SEQ ID NOs: 291-295.

In some embodiments, the open reading frame includes a nucleic acid sequence having at least 85% (e.g., at least 85%, 86%, 87%, 88%, 89%, 90%, 91%, 92%, 93%, 94%, 95%, 96%, 97%, 98%, 99% or 100% sequence identity) sequence identity with the nucleic acid sequence of any one of SEQ ID NOs: 112-174 and 292-300. In some embodiments, the open reading frame includes a nucleic acid sequence having at least 90% (e.g., at least 90%, 91%, 92%, 93%, 94%, 95%, 96%, 97%, 98%, 99% or 100% sequence identity) sequence identity with the nucleic acid sequence of any one of SEQ ID NOs: 112-174 and 292-300. In some embodiments, the open reading frame includes a nucleic acid sequence having at least 95% (e.g., at least 95%, 96%, 97%, 98%, 99% or 100% sequence identity) sequence identity with the nucleic acid sequence of any one of SEQ ID NOs: 112-174 and 292-300. In some embodiments, the open reading frame includes a nucleic acid sequence of any one of SEQ ID NOs: 112-174 and 292-300.

In some embodiments, the coronavirus immunogen is a RBD immunogen having at least 85% (e.g., at least 85%, 86%, 87%, 88%, 89%, 90%, 91%, 92%, 93%, 94%, 95%, 96%, 97%, 98%, 99% or 100% sequence identity) identity with the nucleic acid sequence of any one of SEQ ID NOs: 112-117, 123, 128, 133-138, and 163-174. In some embodiments, the coronavirus immunogen is a RBD immunogen having at least 90% (e.g., at least 90%, 91%, 92%, 93%, 94%, 95%, 96%, 97%, 98%, 99% or 100% sequence identity) identity with the nucleic acid sequence of any one of SEQ ID NOs: 112-117, 123, 128, 133-138, and 163-174. In some embodiments, the coronavirus immunogen is a RBD immunogen having at least 95% (e.g., at least 95%, 96%, 97%, 98%, 99% or 100% sequence identity) identity with the nucleic acid sequence of any one of SEQ ID NOs: 112-117, 123, 128, 133-138, and 163-174. In some embodiments, the coronavirus immunogen is a RBD immunogen having a nucleic acid sequence of any one of SEQ ID NOs: 112-117, 123, 128, 133-138, and 163-174.

In some embodiments, the coronavirus immunogen is a Spike immunogen having at least 85% (e.g., at least 85%, 86%, 87%, 88%, 89%, 90%, 91%, 92%, 93%, 94%, 95%, 96%, 97%, 98%, 99% or 100% sequence identity) identity with the nucleic acid sequence of any one of SEQ ID NOs: 118-122, 124-127, 129-132, 139-162, and 287-291. In some embodiments, the coronavirus immunogen is a Spike immunogen having at least 90% (e.g., at least 90%, 91%, 92%, 93%, 94%, 95%, 96%, 97%, 98%, 99% or 100% sequence identity) identity with the nucleic acid sequence of any one of SEQ ID NOs: 118-122, 124-127, 129-132, 139-162, and 287-291. In some embodiments, the coronavirus immunogen is a Spike immunogen having at least 95% (e.g., at least 95%, 96%, 97%, 98%, 99% or 100% sequence identity) identity with the nucleic acid sequence of any one of SEQ ID NOs: 118-122, 124-127, 129-132, 139-162, and 287-291. In some embodiments, the coronavirus immunogen is a Spike immunogen having a nucleic acid sequence of any one of SEQ ID NOs: 118-122, 124-127, 129-132, 139-162, and 287-291.

In some embodiments, the coronavirus immunogen is a nsp immunogen having at least 85% (e.g., at least 85%, 86%, 87%, 88%, 89%, 90%, 91%, 92%, 93%, 94%, 95%, 96%, 97%, 98%, 99% or 100% sequence identity) identity with the nucleic acid sequence of any one of SEQ ID NOs: 296-300, nsp nsp immunogen having at least 90% (e.g., at least 90%, 91%, 92%, 93%, 94%, 95%, 96%, 97%, 98%, 99% or 100% sequence identity) identity with the nucleic acid sequence of any one of SEQ ID NOs: 296-300. In some embodiments, the coronavirus immunogen is a nsp immunogen having at least 95% (e.g., at least 95%, 96%, 97%, 98%, 99% or 100% sequence identity) identity with the nucleic acid sequence of any one of SEQ ID NOs: 296-300. In some embodiments, the coronavirus immunogen is a nsp immunogen having a nucleic acid sequence of any one of SEQ ID NOs: 296-300.

In some embodiments, the open reading frame encoding the coronavirus immunogen is operably linked to an IRES. In some embodiments, the open reading frame encoding the coronavirus immunogen encodes a second polypeptide. In some embodiments, the coronavirus immunogen and the second polypeptide are separated by a polypeptide linker, a 2A self-cleaving peptide, a protease cleavage site, or 2A self-cleaving peptide in tandem with a protease cleavage site. In some embodiments, the protease cleavage site is a furin cleavage site.

In some embodiments, the circular polyribonucleotide further includes a second open reading frame encoding a second polypeptide operably linked to a second IRES. In some embodiments, the second polypeptide is a polypeptide immunogen. In some embodiments, the second polypeptide is a viral immunogen. In some embodiments, the second polypeptide is a coronavirus immunogen. In some embodiments, the second coronavirus immunogen includes an amino acid sequence having at least 95% identity (e.g., at least 95%, 96%, 97%, 98%, 99%, or 100% identity) to any one of SEQ ID NOs: 1-10, 53, 55, 57, 63-111, and 283-291. In some embodiments, the second coronavirus immunogen includes an amino acid sequence of any one of SEQ ID NOs: 1-10, 53, 55, 57, 63-111, and 283-291. In some embodiments, the second polypeptide is an influenza immunogen.

In some embodiments, the second polypeptide is a polypeptide adjuvant. In some embodiments, the adjuvant is a cytokine, a chemokine, a costimulatory molecule, an innate immune stimulator, a signaling molecule, a transcriptional activator, a cytokine receptor, a bacterial component, or a component of the innate immune system. In some embodiments, the circular polyribonucleotide further includes a non-coding ribonucleic acid sequence that is an innate immune system stimulator. In some embodiments, the innate immune system stimulator is selected from a GU-rich motif, an AU-rich motif, a structured region including dsRNA, or an aptamer.

In another aspect, the disclosure provides a circular polyribonucleotide including a first sequence encoding a coronavirus immunogen and a second sequence encoding a polypeptide adjuvant. In some embodiments, the sequence encoding the coronavirus immunogen is operably linked to a first IRES and the sequence encoding the polypeptide adjuvant is operably linked to a second IRES. In some embodiments, the coronavirus immunogen and the polypeptide adjuvant are encoded by a single open-reading frame operably linked to an IRES. In some embodiments, coronavirus immunogen and the polypeptide adjuvant are separated by a polypeptide linker, a 2A self-cleaving peptide, a protease cleavage site, or 2A self-cleaving peptide in tandem with a protease cleavage site.

In some embodiments, polypeptide adjuvant is a cytokine, a chemokine, a costimulatory molecule, an innate immune stimulator, a signaling molecule, a transcriptional activator, a cytokine receptor, a bacterial component, or a component of the innate immune system. In some embodiments, the second coronavirus immunogen includes an amino acid sequence having at least 95% identity (e.g., at least 95%, 96%, 97%, 98%, 99%, or 100% identity) to any one of SEQ ID NOs: 1-10, 53, 55, 57, 63-111, and 283-291. In some embodiments, the second coronavirus immunogen includes an amino acid sequence of any one of SEQ ID NOs: 1-10, 53, 55, 57, 63-111, and 283-291.

In another aspect, the disclosure provides a circular polyribonucleotide including an open reading frame encoding a coronavirus immunogen and a non-coding ribonucleic acid sequence that is an innate immune system stimulator. In some embodiments, the innate immune system stimulator is selected from a GU-rich motif, an AU-rich motif, a structured region including dsRNA, or an aptamer. In some embodiments, the second coronavirus immunogen includes an amino acid sequence having at least 95% identity (e.g., at least 95%, 96%, 97%, 98%, 99%, or 100% identity) to any one of SEQ ID NOs: 1-10, 53, 55, 57, 63-111, and 283-291. In some embodiments, the second coronavirus immunogen includes an amino acid sequence of any one of SEQ ID NOs: 1-10, 53, 55, 57, 63-111, and 283-291.

In some embodiments, the open reading frame encodes a concatemeric coronavirus immunogen. In some embodiments, the open reading frame comprises between 2-100 coronavirus immunogens connected directly to one another or interspersed by linkers. In other embodiments the immunogen is a concatemeric peptide immunogen composed of multiple peptide epitopes. In some embodiments, the circular polyribonucleotide encodes 2-10 coronavirus immunogens. In some embodiments, the circular polyribonucleotide encodes at least 10, 20, 30, 40, 50, 60, 70, 80, 90 or 100 coronavirus immunogens. In some embodiments, the coronavirus immunogens are separated by a polypeptide linker, a 2A self-cleaving peptide, a protease cleavage site, or 2A self-cleaving peptide in tandem with a protease cleavage site. In some embodiments, the concatemeric coronavirus immunogen includes an amino acid sequence having at least 85% identity (e.g., at least 85%, 86%, 87%, 88%, 89%, 90%, 91%, 92%, 93%, 94%, 95%, 96%, 97%, 98%, 99%, or 100% identity) to any one of SEQ ID NOs: 63-111 and 283-291. In some embodiments, the concatemeric coronavirus immunogen includes an amino acid sequence having at least 95% identity (e.g., at least 95%, 96%, 97%, 98%, 99%, or 100% identity) to any one of SEQ ID NOs: 63-111 and 283-291. In some embodiments, the concatemeric coronavirus immunogen includes an amino acid sequence of any one of SEQ ID NOs: 63-111 and 283-291. In some embodiments, the concatemeric coronavirus immunogen includes a nucleic acid sequence having at least 85% identity (e.g., at least 85%, 86%, 87%, 88%, 89%, 90%, 91%, 92%, 93%, 94%, 95%, 96%, 97%, 98%, 99%, or 100% identity) to any one of SEQ ID NOs: 112-174 and 292-300. In some embodiments, the concatemeric coronavirus immunogen includes a nucleic acid sequence having at least 95% identity (e.g., at least 95%, 96%, 97%, 98%, 99%, or 100% identity) to any one of SEQ ID NOs: 112-174 and 292-300. In some embodiments, the concatemeric coronavirus immunogen includes a nucleic acid sequence of any one of SEQ ID NOs: 112-174 and 292-300.

In another aspect, the disclosure provides a circular polyribonucleotide including a first sequence encoding a coronavirus immunogen and a second sequence encoding a multimerization domain. In some embodiments, the multimerization domain includes a T4 foldon domain. In some embodiments, the multimerization domain includes a ferritin domain. In some embodiments, the multimerization domain includes a β-annulus peptide. In some embodiments, the multimerization domain is at the N-terminus of the coronavirus immunogen. In some embodiments, the multimerization domain is at the C-terminus of the coronavirus immunogen.

In another aspect, the disclosure provides an immunogenic composition including any one of the circular polyribonucleotides described herein, a pharmaceutically acceptable excipient, and is free of any carrier. In another aspect, the disclosure provides an immunogenic composition including any one of the circular polyribonucleotides described herein and a pharmaceutically acceptable carrier or excipient. In some embodiments, the composition further includes a second circular polyribonucleotide. In some embodiments, the second circular polyribonucleotide includes an open reading frame encoding a second polypeptide immunogen. In some embodiments, the second circular polyribonucleotide includes a non-coding ribonucleic acid sequence that is an innate immune system stimulator.

In another aspect, the disclosure provides a linear polyribonucleotide including an open reading frame encoding a coronavirus immunogen, wherein the coronavirus immunogen includes an amino acid sequence having at least 85% (e.g., at least 85%, 86%, 87%, 88%, 89%, 90%, 91%, 92%, 93%, 94%, 95%, 96%, 97%, 98%, 99%, or 100%) sequence identity with the amino acid sequence of any one of SEQ ID NOs: 63-111 and 283-291. In some embodiments, the coronavirus immunogen includes an amino acid sequence having at least 90% (e.g., at least 90%, 91%, 92%, 93%, 94%, 95%, 96%, 97%, 98%, 99%, or 100%) sequence identity with the amino acid sequence of any one of SEQ ID NOs: 63-111 and 283-291. In some embodiments, the coronavirus immunogen includes an amino acid sequence having at least 95% (e.g., at least 95%, 96%, 97%, 98%, 99%, or 100%) sequence identity with the amino acid sequence of any one of SEQ ID NOs: 63-111 and 283-291. In some embodiments, the coronavirus immunogen includes an amino acid sequence having an amino acid sequence of any one of SEQ ID NOs: 63-111 and 283-291.

In some embodiments, the open reading frame includes a nucleic acid sequence having at least 85% (e.g., at least 85%, 86%, 87%, 88%, 89%, 90%, 91%, 92%, 93%, 94%, 95%, 96%, 97%, 98%, 99%, or 100%) sequence identity with the nucleic acid sequence of any one of SEQ ID NOs: 112-174 and 292-300. In some embodiments, the open reading frame includes a nucleic acid sequence having at least 90% (e.g., at least 90%, 91%, 92%, 93%, 94%, 95%, 96%, 97%, 98%, 99%, or 100%) sequence identity with the nucleic acid sequence of any one of SEQ ID NOs: 112-174 and 292-300. In some embodiments, the open reading frame includes a nucleic acid sequence having at least 95% (e.g., at least 95%, 96%, 97%, 98%, 99%, or 100%) sequence identity with the nucleic acid sequence of any one of SEQ ID NOs: 112-174 and 292-300. In some embodiments, the open reading frame includes a nucleic acid sequence having a nucleic acid sequence of any one of SEQ ID NOs: 112-174 and 292-300.

In some embodiments, the open reading frame encoding the coronavirus immunogen is operably linked to an IRES. In some embodiments, the open reading frame encoding the coronavirus immunogen encodes a second polypeptide. In some embodiments, the coronavirus immunogen and the second polypeptide are separated by a polypeptide linker, a 2A self-cleaving peptide, a protease cleavage site, or 2A self-cleaving peptide in tandem with a protease cleavage site. In some embodiments, the protease cleavage site is a furin cleavage site.

In some embodiments, the circular polyribonucleotide further includes a second open reading frame encoding a second polypeptide operably linked to a second IRES. In some embodiments, the second polypeptide is a polypeptide immunogen. In some embodiments, the second polypeptide is a coronavirus immunogen. In some embodiments, the second polypeptide is a polypeptide adjuvant. In some embodiments, the adjuvant is a cytokine, a chemokine, a costimulatory molecule, an innate immune stimulator, a signaling molecule, a transcriptional activator, a cytokine receptor, a bacterial component, or a component of the innate immune system. In some embodiments, the linear polyribonucleotide further includes a non-coding ribonucleic acid sequence that is an innate immune system stimulator. In some embodiments, the innate immune system stimulator is selected from a GU-rich motif, an AU-rich motif, a structured region including dsRNA, or an aptamer.

In another aspect the disclosure provides a linear polyribonucleotide including a first sequence encoding a coronavirus immunogen and a second sequence encoding a multimerization domain. In some embodiments, the multimerization domain includes a T4 foldon domain. In some embodiments, the multimerization domain includes a ferritin domain. In some embodiments, the multimerization domain includes a β-annulus peptide. In some embodiments, the multimerization domain is at the N-terminus of the coronavirus immunogen. In some embodiments, the multimerization domain is at the C-terminus of the coronavirus immunogen.

In another aspect, the disclosure provides an immunogenic composition including any one of the linear polyribonucleotides described herein and a pharmaceutically acceptable excipient and is free of any carrier. In another aspect, the disclosure provides an immunogenic composition including any one of the linear polyribonucleotides described herein and a pharmaceutically acceptable carrier and excipient. In some embodiments, the composition further includes a second linear polyribonucleotide. In some embodiments, the second linear polyribonucleotide includes an open reading frame encoding a second polypeptide immunogen. In some embodiments, the second linear polyribonucleotide includes an open reading frame encoding a polypeptide adjuvant. In some embodiments, the second linear polyribonucleotide includes a non-coding ribonucleic acid sequence that is an innate immune system stimulator.

In another aspect, the disclosure provides a method of inducing an immune response against a coronavirus immunogen in a non-human animal or human subject by: a) administering any one of the immunogenic compositions described herein to the non-human animal or human subject, and b) collecting antibodies against the coronavirus immunogen from the non-human animal or human subject. In some embodiments, further including administering an adjuvant to the non-human animal or human subject.

In another aspect, the disclosure provides a method of treating a subject who has or is suspected to have a SARS-CoV-2 infection including administering to the subject any one of the circular polyribonucleotides or immunogenic compositions described herein.

In another aspect, the disclosure provides a method of preventing a SARS-CoV-2 infection in a subject including administering to the subject any one of the circular polyribonucleotide or immunogenic compositions described herein. In some embodiments, the human subject is at risk for a SARS-CoV-2 infection. In some embodiments, the human subject is a human over 50 years old, an immune-compromised human, a human with a chronic health condition, or a health care worker. In some embodiments, administering the circular polyribonucleotide or immunogenic composition decreases the frequency or severity of symptoms associated with a SARS-CoV-2 infection. In some embodiments, the subject is a human subject. In some embodiments, the method further includes administering an adjuvant to the subject.

### Definitions

The present invention will be described with respect to particular embodiments and with reference to certain figures, but the invention is not limited thereto but only by the claims. Terms as set forth hereinafter are generally to be understood in their common sense unless indicated otherwise.

As used herein, the term “adaptive immune response” means either a humoral or cell-mediated immune response. For purposes of the present disclosure, a “humoral immune response” refers to an immune response mediated by antibody molecules, while a “cellular immune response” is one mediated by T-lymphocytes and/or other white blood cells.

As used herein, the term “adjuvant” refers to a composition (e.g., a compound, polypeptide, nucleic acid, or lipid) that increases an immune response, for example, increases a specific immune response against an immunogen. Increasing an immune response includes intensification or broadening the specificity of either or both antibody and cellular immune responses.

As used herein, the terms “circRNA,” “circular polyribonucleotide,” “circular RNA,” and “circular polyribonucleotide molecule” are used interchangeably and mean a polyribonucleotide molecule that has a structure having no free ends (i.e., no free 3′ and/or 5′ ends), for example a polyribonucleotide molecule that forms a circular or end-less structure through covalent (e.g., covalently closed) or non-covalent bonds. The circular polyribonucleotide may be covalently closed polyribonucleotide.

As used herein, the term “circularization efficiency” is a measurement of resultant circular polyribonucleotide versus its non-circular starting material.

The term “diluent” means a vehicle comprising an inactive solvent in which a composition described herein (e.g., a composition comprising a circular polyribonucleotide) may be diluted or dissolved. A diluent can be an RNA solubilizing agent, a buffer, an isotonic agent, or a mixture thereof. A diluent can be a liquid diluent or a solid diluent. Non-limiting examples of liquid diluents include water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and 1,3-butanediol. Non-limiting examples of solid diluents include calcium carbonate, sodium carbonate, calcium phosphate, dicalcium phosphate, calcium sulfate, calcium hydrogen phosphate, sodium phosphate, lactose, sucrose, cellulose, microcrystalline cellulose, kaolin, mannitol, sorbitol, inositol, sodium chloride, dry starch, cornstarch, or powdered sugar.

As used herein, the term “epitope” refers to a portion or the whole of an immunogen that is recognized, targeted, or bound by an antibody or T cell receptor. An epitope can be a linear epitope, for example, a contiguous sequence of nucleic acids or amino acids. An epitope can be a conformational epitope, for example, an epitope that contains amino acids that form an epitope in the folded conformation of the protein. A conformational epitope can contain non-contiguous amino acids from a primary amino acid sequence. As another example, a conformational epitope includes nucleic acids that form an epitope in the folded conformation of an immunogenic sequence based on its secondary structure or tertiary structure.

As used herein, the term “expression sequence” is a nucleic acid sequence that encodes a product, e.g., a polypeptide (e.g., an immunogen), or a regulatory nucleic acid. An exemplary expression sequence that codes for a polypeptide can comprise a plurality of nucleotide triads, each of which can code for an amino acid and is termed as a “codon”.

As used herein, the term “fragment” with respect to a polypeptide or a nucleic acid sequence, e.g., a polypeptide immunogen or a nucleic acid sequence encoding a polypeptide immunogen, refers to a continuous, less than a whole portion of a sequence of the polypeptide or the nucleic acid. A fragment of a polypeptide immunogen or a nucleic acid sequence encoding a polypeptide immunogen, for instance, refers to continuous, less than a whole fraction (e.g., at least 20%, 30%, 40%, 50%, 60%, 70%, 80%, 90%, 95%, or 99% of the entire length) of the sequence such as a sequence disclosed herein. It is understood that all the present disclosure contemplates fragments (e.g., immunogenic fragments) of all immunogens disclosed herein.

As used herein, the term “GC content” refers to the percentage of guanine (G) and cytosine (C) in a nucleic acid sequence. The formula for calculation of the GC content is (G+C)/(A+G+C+U)×100% (for RNA) or (G+C)/(A+G+C+T)×100% (for DNA). Likewise, the term “uridine content” refers to the percentage of uridine (U) in a nucleic acid sequence. The formula for calculation of the uridine content is U/(A+G+C+U)×100%. Likewise, the term “thymidine content” refers to the percentage of thymidine (T) in a nucleic acid sequence. The formula for calculation of the thymidine content is T/(A+G+C+T)×100%.

As used herein, the term “innate immune system stimulator” refers to a substance that induces an innate immunological response, in part, by inducing expression of one or more genes involved in innate immunity, including, but not limited to, a type I interferon (e.g., IFNα, INFβ, and/or IFNγ), a pro-inflammatory cytokine (e.g., IL-1, IL-12, IL-18, TNF-α, and/or GM-CSF), retinoic-acid inducible gene-I (RIG-1, also known as DDX58), melanoma-differentiation-associated gene 5 (MDA5, also known as IFIH1), 2′-5′ oligoadenylate synthase 1 (OAS 1), OAS-like protein (OASL), and/or protein kinase R (PKR). An innate immune system stimulator may act as an adjuvant, e.g., when administered in combination with or formulated with a ribonucleotide that encodes an immunogen. An innate immune system stimulator may be a separate molecule entity (e.g., not encoded by or incorporated as a sequence in a polyribonucleotide), for example, STING (e.g., caSTING), TLR3, TLR4, TLR9, TLR7, TLR8, TLR7, RIG-I/DDX58, and MDA-5/IFIH1 or a constitutively active mutant thereof. An innate immune system stimulator may be encoded by (e.g., expressed from) a polyribonucleotide. A polyribonucleotide may alternately or further include a ribonucleotide sequence that acts as an innate immune system stimulator (e.g., GU-rich motif, an AU-rich motif, a structured region including dsRNA, or an aptamer).

As used herein, the terms “human antibody,” “human immunoglobulin,” and “human polyclonal antibody” are used interchangeably and mean an antibody or antibodies produced in a non-human animal that is otherwise indistinguishable from antibody produced in a human vaccinated by the same circular RNA preparation. This is in contrast to “humanized antibodies” which are modified to have human characteristics, such as through generation of chimeras, but that maintain attributes of the host animal in which they are produced. Because human antibody made according to the method disclosed herein is comprised of IgG that are fully human, no enzymatic treatment is needed to eliminate the risk of anaphylaxis and serum sickness associated with heterologous species IgG.

As used herein, the term “immunogen” refers to any molecule or molecular structure that includes one or more epitopes recognized, targeted, or bound by an antibody or a T cell receptor. In particular, an immunogen induces an immune response in a subject (e.g., is immunogenic as defined herein). An immunogen is capable of inducing an immune response in a subject, wherein the immune response refers to a series of molecular, cellular, and organismal events that are induced when an immunogen is encountered by the immune system. The immune response may be humoral and/or cellular immune response. These may include the production of antibodies and the expansion of B- and T-cells. To determine whether an immune response has occurred and to follow its course, the immunized subject can be monitored for the appearance of immune reactants directed at the specific immunogen. Immune responses to most immunogens induce the production of both specific antibodies and specific effector T cells. In some embodiments, the immunogen is foreign to a host.

In some embodiments, the immunogen is not foreign to a host. An immunogen may include all or a portion of a polypeptide, a polysaccharide, a polynucleotide, or a lipid. An immunogen may also be a mixed polypeptide, polysaccharide, polynucleotide, and/or lipid. For example, an immunogen may be a polypeptide that has been translationally modified. A “polypeptide immunogen” refers to an immunogen that includes a polypeptide. A polypeptide immunogen may also include one or more post-translational modifications, and/or may form a complex with one or more additional molecules, and/or may adopt a tertiary or quaternary structure, each of which may determine or affect the immunogenicity of the polypeptide.

As used herein, the term “immunogenic” is a potential to induce a response to a substance in a particular immune response assay above a pre-determined threshold. The assay can be, e.g., expression of certain inflammatory markers, production of antibodies, or an assay for immunogenicity as described herein. In some embodiments, an immune response may be induced when an immune system of an organism or a certain type of immune cells are exposed to an immunogen.

An immunogenic response may be assessed may evaluating the antibodies in the plasma or serum of a subject using a total antibody assay, a confirmatory test, titration and isotyping of the antibodies, and neutralizing antibody assessment. A total antibody assay measures all the antibodies generated as part of the immune response in the serum or plasma of a subject that has been administered the immunogen. The most commonly used test to detect antibodies is an ELISA (enzyme-linked immunosorbent assay), which detects antibodies in the tested serum that bind to the antibody of interest, including IgM, IgD, IgG, IgA, and IgE. An immunogenic response can be further assessed by a confirmatory assay. Following a total antibody assessment, a confirmatory assay may be used to confirm the results of the total antibody assay. A competition assay may be used to confirm that antibody is specifically binding to target and that the positive finding in the screening assay is not a result of non-specific interactions of the test serum or detection reagent with other materials in the assay.

An immunogenic response can be assessed by isotyping and titration. An isotyping assay may be used to assess only the relevant antibody isotypes. For example, the expected isotypes may be IgM and IgG which may be specifically detected and quantified by isotyping and titration, and then compared to the total antibodies present.

An immunogenic response can be assessed by a neutralizing antibody assay (nAb). A neutralizing antibody assay (nAb) may be used to determine if the antibodies produced in response to the immunogen neutralized the immunogen thereby inhibiting the immunogen from having an effect on the target and leading to abnormal pharmacokinetic behaviors. An nAb assay is often a cell-based assay where the target cells are incubated with the antibody. A variety of cell based nAb assays may be used including but not limited to Cell Proliferation, Viability, Antibody-Dependent Cell-Mediated Cytotoxicity (ADCC), Complement-Dependent Cytotoxicity (CDC), Cytopathic Effect Inhibition (CPE), Apoptosis, Ligand Stimulated Cell Signaling, Enzyme Activity, Reporter Gene Assays, Protein Secretion, Metabolic Activity, Stress and Mitochondrial Function. Detection readouts include Absorbance, Fluorescence, Luminescence, Chemiluminescence, or Flow Cytometry. A ligand-binding assay may also be used to measure the binding affinity of an immunogen and an antibody in vitro to evaluate neutralization efficacy.

Furthermore, induction of a cellular immune response may be assessed by measuring T cell activation in a subject using cellular markers on T cells obtained from the subject. A blood sample, lymph node biopsy, or tissue sample can be collected from a subject and T cells from the sample evaluated for one or more (e.g., 2, 3, 4 or more) activation markers: CD25, CD71, CD26, CD27, CD28, CD30, CD154, CD40 L, CD134, CD69, CD62 L or CD44. T cell activation can also be assessed using the same methods in an in vivo animal model. This assay can also be performed by adding an immunogen to T cells in vitro (e.g., T cells obtained from a subject, animal model, repository, or commercial source) and measuring the aforementioned markers to evaluate T cell activation. Similar approaches can be used to assess the effect of and on activation of other immune cells, such as eosinophils (markers: CD35, CD11 b, CD66, CD69 and CD81), dendritic cells (makers: IL-8, MHC class II, CD40, CD80, CD83, and CD86), basophils (CD63, CD13, CD4, and CD203c), and neutrophils (CD11 b, CD35, CD66b and CD63). These markers can be assessed using flow cytometry, immunohistochemistry, in situ hybridization, and other assays that allow for measurement of cellular markers. Comparing results from before and after administration of an immunogen can be used to determine its effect.

As used herein, the term “impurity” is an undesired substance present in a composition, e.g., a pharmaceutical composition as described herein. In some embodiments, an impurity is a process-related impurity. In some embodiments, an impurity is a product-related substance other than the desired product in the final composition, e.g., other than the active drug ingredient, e.g., circular or linear polyribonucleotide, as described herein. As used herein, the term “process-related impurity” is a substance used, present, or generated in the manufacturing of a composition, preparation, or product that is undesired in the final composition, preparation, or product other than the linear polyribonucleotides described herein. In some embodiments, the process-related impurity is an enzyme used in the synthesis or circularization of polyribonucleotides. As used herein, the term “product-related substance” is a substance or byproduct produced during the synthesis of a composition, preparation, or product, or any intermediate thereof. In some embodiments, the product-related substance is deoxyribonucleotide fragments. In some embodiments, the product-related substance is deoxyribonucleotide monomers. In some embodiments, the product-related substance is one or more of: derivatives or fragments of polyribonucleotides described herein, e.g., fragments of 10, 9, 8, 7, 6, 5, or 4 ribonucleic acids, monoribonucleic acids, diribonucleic acids, or triribonucleic acids.

As used herein, the term “inducing an immune response” refers to initiating, amplifying, or sustaining an immune response by a subject. Inducing an immune response may refer to an adaptive immune response or an innate immune response. The induction of an immune response may be measured as discussed above.

As used herein, the terms “linear RNA,” “linear polyribonucleotide,” and “linear polyribonucleotide molecule” are used interchangeably and mean a monoribonucleotide molecule or polyribonucleotide molecule having a 5′ and 3′ end. One or both of the 5′ and 3′ ends may be free ends or joined to another moiety. In some embodiments, the linear RNA has a 5′ end or 3′ end that is modified or protected from degradation (e.g., by a 5′ end protectant or a 3′ end protectant). In some embodiments, the linear RNA has non-covalently linked 5′ or 3′ ends. A linear RNA can be used as a starting material for circularization through, for example, splint ligation, or chemical, enzymatic, ribozyme- or splicing-catalyzed circularization methods.

As used herein, the term “linear counterpart” is a polyribonucleotide molecule (and its fragments) having the same or similar nucleotide sequence (e.g., 100%, 95%, 90%, 85%, 80%, 75%, or any percentage therebetween sequence similarity) as a circular polyribonucleotide and having two free ends (i.e., the uncircularized version (and its fragments) of the circularized polyribonucleotide). In some embodiments, the linear counterpart (e.g., a pre-circularized version) is a polyribonucleotide molecule (and its fragments) having the same or similar nucleotide sequence (e.g., 100%, 95%, 90%, 85%, 80%, 75%, or any percentage therebetween sequence similarity) and same or similar nucleic acid modifications as a circular polyribonucleotide and having two free ends (i.e., the uncircularized version (and its fragments) of the circularized polyribonucleotide). In some embodiments, the linear counterpart is a polyribonucleotide molecule (and its fragments) having the same or similar nucleotide sequence (e.g., 100%, 95%, 90%, 85%, 80%, 75%, or any percentage therebetween sequence similarity) and different or no nucleic acid modifications as a circular polyribonucleotide and having two free ends (i.e., the uncircularized version (and its fragments) of the circularized polyribonucleotide). In some embodiments, a fragment of the polyribonucleotide molecule that is the linear counterpart is any portion of linear counterpart polyribonucleotide molecule that is shorter than the linear counterpart polyribonucleotide molecule. In some embodiments, the linear counterpart further comprises a 5′ cap. In some embodiments, the linear counterpart further comprises a poly adenosine tail. In some embodiments, the linear counterpart further comprises a 3′ UTR. In some embodiments, the linear counterpart further comprises a 5′ UTR.

As used herein, the term “modified ribonucleotide” is a nucleotide with at least one modification to the sugar, the nucleobase, or the internucleoside linkage.

As used herein, the term “multimerization domain” refers to a polypeptide domain that self-assembles to form multimers (e.g., dimers, trimers, tetramers, or oligomers). In particular embodiments, a multimerization domain can be fused to a polypeptide (e.g., a polypeptide immunogen). In such instances, fusion to a multimerization domain results in the formation of a multimeric immunogen complex having more than one immunogen upon expression of the polypeptide including an immunogen covalently attached to a multimerization domain.

As used herein, the term “naked,” “naked delivery,” and its cognates means a formulation for delivery to a cell without the aid of a carrier and without covalent modification to a moiety that aids in delivery to a cell. A naked delivery formulation is free from any transfection reagents, cationic carriers, carbohydrate carriers, nanoparticle carriers, or protein carriers. For example, naked delivery formulation of a circular polyribonucleotide is a formulation that comprises a circular polyribonucleotide without covalent modification and is free from a carrier. A naked delivery formulation may comprise non-carrier pharmaceutical excipients or diluents.

As used herein, the term “naked delivery” means a formulation for delivery to a cell without the aid of a carrier and without covalent modification to a moiety that aids in delivery to a cell. A naked delivery formulation is free from any transfection reagents, cationic carriers, carbohydrate carriers, nanoparticle carriers, or protein carriers. For example, naked delivery formulation of a circular polyribonucleotide is a formulation that includes a circular polyribonucleotide without covalent modification and is free from a carrier.

As used herein, the terms “nicked RNA,” “nicked linear polyribonucleotide,” and “nicked linear polyribonucleotide molecule” are used interchangeably and mean a polyribonucleotide molecule having a 5′ and 3′ end that results from nicking or degradation of a circular RNA.

As used herein, the term “non-circular RNA” means total nicked RNA and linear RNA.

The term “pharmaceutical composition” is intended to also disclose that the circular polyribonucleotide included within a pharmaceutical composition can be used for the treatment of the human or animal body by therapy. It is thus meant to be equivalent to “a circular polyribonucleotide for use in therapy”.

The term “polynucleotide” as used herein means a molecule including one or more nucleic acid subunits, or nucleotides, and can be used interchangeably with “nucleic acid” or “oligonucleotide”. A polynucleotide can include one or more nucleotides selected from adenosine (A), cytosine (C), guanine (G), thymine (T) and uracil (U), or variants thereof. A nucleotide can include a nucleoside and at least 1, 2, 3, 4, 5, 6, 7, 8, 9, 10, or more phosphate (PO3) groups. A nucleotide can include a nucleobase, a five-carbon sugar (either ribose or deoxyribose), and one or more phosphate groups. Ribonucleotides are nucleotides in which the sugar is ribose. Polyribonucleotides or ribonucleic acids, or RNA, can refer to macromolecules that include multiple ribonucleotides that are polymerized via phosphodiester bonds. Deoxyribonucleotides are nucleotides in which the sugar is deoxyribose.

“Polydeoxyribonucleotides,” “deoxyribonucleic acids,” and “DNA” mean macromolecules that include multiple deoxyribonucleotides that are polymerized via phosphodiester bonds. A nucleotide can be a nucleoside monophosphate or a nucleoside polyphosphate. A nucleotide means a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), uridine triphosphate (dUTP) and deoxythymidine triphosphate (dTTP) dNTPs, that include detectable tags, such as luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). In some examples, a polynucleotide is deoxyribonucleic acid (DNA), ribonucleic acid (RNA), or derivatives or variants thereof. In some cases, a polynucleotide is a short interfering RNA (siRNA), a microRNA (miRNA), a plasmid DNA (pDNA), a short hairpin RNA (shRNA), small nuclear RNA (snRNA), messenger RNA (mRNA), precursor mRNA (pre-mRNA), antisense RNA (asRNA), to name a few, and encompasses both the nucleotide sequence and any structural embodiments thereof, such as single-stranded, double-stranded, triple-stranded, helical, hairpin, etc. In some cases, a polynucleotide molecule is circular. A polynucleotide can have various lengths. A nucleic acid molecule can have a length of at least about 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. A polynucleotide can be isolated from a cell or a tissue. As embodied herein, the polynucleotide sequences may include isolated and purified DNA/RNA molecules, synthetic DNA/RNA molecules, and synthetic DNA/RNA analogs.

Polynucleotides, e.g., polyribonucleotides or polydeoxyribonucleotides, may include one or more nucleotide variants, including nonstandard nucleotide(s), non-natural nucleotide(s), nucleotide analog(s) and/or modified nucleotides. Examples of modified nucleotides include, but are not limited to diaminopurine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, wybutoxosine, pseudo uracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid(v), 5-methyl-2-thiouracil, 3-(3-amino-3-N-2-carboxypropyl) uracil, 3-(3-amino-3-carboxypropyl)uridine 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates). Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as amino ally 1-dUTP (aa-dUTP) and aminohexhylacrylamide-dCTP (aha-dCTP) to allow covalent attachment of amine reactive moieties, such as N-hydroxy succinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs compatible with natural and mutant polymerases for de novo and/or amplification synthesis are described in Betz K, Malyshev D A, Lavergne T, Welte W, Diederichs K, Dwyer T J, Ordoukhanian P, Romesberg F E, Marx A. NAT. CHEM. BIOL. 2012 July; 8(7):612-4, which is herein incorporated by reference for all purposes.

As used herein, “polypeptide” means a polymer of amino acid residues (natural or unnatural) linked together most often by peptide bonds. The term, as used herein, refers to proteins, polypeptides, and peptides of any size, structure, or function. Polypeptides can include gene products, naturally occurring polypeptides, synthetic polypeptides, homologs, orthologs, paralogs, fragments and other equivalents, variants, and analogs of the foregoing. A polypeptide can be a single molecule or may be a multi-molecular complex such as a dimer, trimer, or tetramer. They can also comprise single chain or multichain polypeptides such as antibodies or insulin and can be associated or linked. Most commonly disulfide linkages are found in multichain polypeptides. The term polypeptide can also apply to amino acid polymers in which one or more amino acid residues are an artificial chemical analogue of a corresponding naturally occurring amino acid.

As used herein, the term “prevent” means to reduce the likelihood of developing a disease, disorder, or condition, or alternatively, to reduce the severity or frequency of symptoms in a subsequently developed disease or disorder. A therapeutic agent can be administered to a subject who is at increased risk of developing a disease or disorder relative to a member of the general population in order to prevent the development of, or lessen the severity of, the disease or condition. A therapeutic agent can be administered as a prophylactic, e.g., before development of any symptom or manifestation of a disease or disorder.

As used interchangeably herein, the terms “polyA” and “polyA sequence” refer to an untranslated, contiguous region of a nucleic acid molecule of at least 5 nucleotides in length and consisting of adenosine residues. In some embodiments, a polyA sequence is at least 10 (SEQ ID NO: 330), at least 15 (SEQ ID NO: 331), at least 20 (SEQ ID NO: 332), at least 30 (SEQ ID NO: 333), at least 40 (SEQ ID NO: 334), or at least 50 (SEQ ID NO: 335) nucleotides in length. In some embodiments, a polyA sequence is located 3′ to (e.g., downstream of) an open reading frame (e.g., an open reading frame encoding a polypeptide), and the polyA sequence is 3′ to a termination element (e.g., a Stop codon) such that the polyA is not translated. In some embodiments, a polyA sequence is located 3′ to a termination element and a 3′ untranslated region.

As used herein, the term “regulatory element” is a moiety, such as a nucleic acid sequence, that modifies expression of an expression sequence within the circular polyribonucleotide.

As used herein, the term “replication element” is a sequence and/or motif useful for replication or that initiates transcription of the circular polyribonucleotide.

As used herein, the term “RNA equivalent” refers to an RNA sequence that is the RNA equivalent of a DNA sequence. An RNA equivalent of a DNA sequence therefore refers to a DNA sequence in which each of the thymidine (T) residues is replaced by a uridine (U) residue. For example, the disclosure provides DNA sequence for ribozymes identified by bioinformatics methods. The disclosure specifically contemplates that any of these DNA sequences may be converted to the corresponding RNA sequence and included in an RNA molecule described herein.

As used herein, the term “sequence identity” is determined by alignment of two peptide or two nucleotide sequences using a global or local alignment algorithm. Sequences may then be referred to as “substantially identical” or “essentially similar” when they (when optimally aligned by for example the programs GAP or BESTFIT using default parameters) share at least a certain minimal percentage of sequence identity. GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is a nwsgapdna.cmp scoring matrix and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or EmbossWin version 2.10.0 (using the program “needle”). Alternatively, or additionally, percent identity may be determined by searching against databases, using algorithms such as FASTA, BLAST, etc. Sequence identity refers to the sequence identity over the entire length of the sequence.

A “signal sequence” refers to a polypeptide sequence, e.g., between 10 and 45 amino acids in length, that is present at the N-terminus of a polypeptide sequence of a nascent protein which targets the polypeptide sequence to the secretory pathway.

As used herein, the terms “treat” and “treating” refer to a therapeutic treatment of a disease or disorder (e.g., an infectious disease, a cancer, a toxicity, or an allergic reaction) in a subject. The effect of treatment can include reversing, alleviating, reducing severity of, curing, inhibiting the progression of, reducing the likelihood of recurrence of the disease or one or more symptoms or manifestations of the disease or disorder, stabilizing (i.e., not worsening) the state of the disease or disorder, and/or preventing the spread of the disease or disorder as compared to the state and/or the condition of the disease or disorder in the absence of the therapeutic treatment.

As used herein, the term “termination element” is a moiety, such as a nucleic acid sequence, that terminates translation of the expression sequence in the circular polyribonucleotide.

As used herein, the term “total ribonucleotide molecules” means the total amount of any ribonucleotide molecules, including linear polyribonucleotide molecules, circular polyribonucleotide molecules, monomeric ribonucleotides, other polyribonucleotide molecules, fragments thereof, and modified variations thereof, as measured by total mass of the ribonucleotide molecules

As used herein, the term “translation efficiency” is a rate or amount of protein or peptide production from a ribonucleotide transcript. In some embodiments, translation efficiency can be expressed as amount of protein or peptide produced per given amount of transcript that codes for the protein or peptide, e.g., in a given period of time, e.g., in a given translation system, e.g., an in vitro translation system like rabbit reticulocyte lysate, or an in vivo translation system like a eukaryotic cell or a prokaryotic cell.

As used herein, the term “translation initiation sequence” is a nucleic acid sequence that initiates translation of an expression sequence in the circular polyribonucleotide.

As used herein, a “variant” refers to a polypeptide which includes at least one alteration, e.g., a substitution, insertion, deletion, and/or fusion, at one or more residue positions, as compared to the parent or wild-type polypeptide. A variant may include between 1 and 10, 10 and 20, 20 and 50, 50 and 100, or more alterations.

### BRIEF DESCRIPTION OF THE DRAWINGS

FIG. 1 shows exemplary circular polyribonucleotides comprising a sequence encoding a coronavirus immunogen (e.g., a spike protein, a receptor binding domain (RBD) protein of a spike protein).

FIG. 2 shows exemplary polyribonucleotide constructs encoding a coronavirus immunogen and one or more multimerization domains.

FIG. 3 is a schematic of an exemplary circular RNA that includes two expression sequences, each expression sequence operably connected to an IRES, and where at least one expression sequence is a coronavirus immunogen.

FIG. 4 is a schematic of an exemplary circular RNA that includes two expression sequences, separated by a cleavage domain (e.g., a 2A, a furin site, or a furin-2A), where at least one expression sequence is a coronavirus immunogen, and all are operably linked to an IRES.

FIG. 5 shows a schematic of a plurality of circular RNAs, where a first circular RNA includes an ORF encoding a coronavirus immunogen and a second circular RNA includes an ORF encoding either a second immunogen or a polypeptide adjuvant.

FIG. 6A shows multi-immunogen expression from a circular polyribonucleotide. RBD immunogen expression was detected from circular RNAs encoding a SARSs-CoV-2 RBD immunogen and GLuc.

FIG. 6B shows multi-immunogen expression from a circular polyribonucleotide. GLuc activity was detected from circular RNAs encoding a SARS-CoV-2 RBD immunogen and GLuc.

FIG. 7A demonstrates immunogenicity of multiple immunogen immunogens from circular RNAs in mouse model. Mice were vaccinated with a first circular RNA encoding a SARS-CoV-2 RBD immunogen and a second circular RNA encoding GLuc. Anti-RBD antibodies were obtained at 17 days after injection.

FIG. 7B demonstrates immunogenicity of multiple immunogens from circular RNAs in mouse model. Mice were vaccinated with a first circular RNA encoding a SARS-CoV-2 RBD immunogen and a second circular RNA encoding GLuc. GLuc activity was detected at 2 days after injection.

FIG. 8A demonstrates immunogenicity of multiple immunogens from circular RNAs in mouse model. Mice were vaccinated with a first circular RNA encoding a SARS-CoV-2 RBD immunogen and a second circular RNA encoding Influenza hemagglutinin (HA) immunogen. Anti-RBD antibodies were obtained at 17 days after injection.

FIG. 8B demonstrates immunogenicity of multiple immunogens from circular RNAs in mouse model. Mice were vaccinated with a first circular RNA encoding a SARS-CoV-2 RBD immunogen and a second circular RNA encoding Influenza hemagglutinin (HA) immunogen. Anti-HA antibodies were obtained at 17 days after injection.

FIG. 9A demonstrates immunogenicity of multiple immunogens from circular RNAs in mouse model. Mice were vaccinated with a first circular RNA encoding a SARS-CoV-2 Spike immunogen and a second circular RNA encoding Influenza hemagglutinin (HA) immunogen. Anti-RBD (domain of Spike) antibodies were obtained at 17 days after injection.

FIG. 9B demonstrates immunogenicity of multiple immunogens from circular RNAs in mouse model. Mice were vaccinated with a first circular RNA encoding a SARS-CoV-2 Spike immunogen and a second circular RNA encoding Influenza hemagglutinin (HA) immunogen. Anti-HA antibodies were obtained at 17 days after injection.

FIG. 10 demonstrates an anti-HA antibody response in mice administered circular RNA encoding multiple immunogens. Mice were administered a circular RNA encoding: a SARS-CoV-2 RBD immunogen, a SARS-CoV-2 Spike immunogen, an Influenza HA immunogen, a SARS-CoV-2 RBD immunogen and an Influenza HA immunogen, a SARS-CoV-2 RBD immunogen and a GLuc protein, or a SARS-CoV-2 RBD immunogen and a SARS-CoV-2 Spike immunogen. A hemagglutination inhibition assay (HAI) was used to measure anti-Influenza HA antibodies. FIG. 10 shows HAI titer in samples that were administered circular RNA preparations encoding the Influenza HA immunogen when it was administered alone or when administered in combination with SARS-CoV-2 immunogens e.g., RBD or Spike.

FIG. 11 shows IL-12, measured using an IL-12 specific ELISA, was expressed from circular RNA in mammalian cells. A circular RNA encoding a SARS-CoV-2 RBD immunogen was included as a negative control.

FIG. 12A shows IL-12 expression was detected in serum at 2 days after injection with a circular RNA preparation including a first circular RNA encoding IL-12 and a second circular RNA encoding a SARS-CoV-2 RBD immunogen, in a mouse model. Injection with PBS or with a preparation including only the circular RNA encoding a SARS-CoV-2 RBD immunogen were included as controls.

FIG. 12B shows an increase in serum IFN-γ (directly downstream of IL12 signaling) was detected in serum at 2 days after injection with a circular RNA preparation including a first circular RNA encoding IL-12 and a second circular RNA encoding a SARS-CoV-2 RBD immunogen, in a mouse model. Injection with PBS or with a preparation including only the circular RNA encoding a SARS-CoV-2 RBD immunogen were included as controls.

FIG. 13A shows that administration of a circular RNA preparation including a first circular RNA encoding IL-12 and a second circular RNA encoding a SARS-CoV-2 RBD immunogen increased the number of SARS-CoV-2 RBD specific CD4 T cells. Administration of PBS or a preparation including only the circular RNA encoding a SARS-CoV-2 RBD immunogen were included as controls. Asterisks denotes statistical significance as determined by a two-way RM ANOVA protected Tukey's post hoc test.

FIG. 13B shows that administration of a circular RNA preparation including a first circular RNA encoding IL-12 and a second circular RNA encoding a SARS-CoV-2 RBD immunogen produced no change in the number of RBD specific CD8 T cells. Administration of PBS or a preparation including only the circular RNA encoding a SARS-CoV-2 RBD immunogen were included as controls.

FIG. 13C shows that administration of a circular RNA preparation including a first circular RNA encoding IL-12 and a second circular RNA encoding SARS-CoV-2 RBD immunogen increased the amount of IFN-γ production by CD4 T cells. Administration of PBS or a preparation including only the circular RNA encoding a SARS-CoV-2 RBD immunogen were included as controls. Asterisks denotes statistical significance as determined by unpaired t-test.

FIG. 13D shows that administration of a circular RNA preparation including a first circular RNA encoding IL-12 and a second circular RNA encoding a SARS-CoV-2 RBD immunogen increased the amount of IFN-γ production by CD8 T cells. Administration of PBS or a preparation including only the circular RNA encoding a SARS-CoV-2 RBD immunogen were included as controls. Asterisks denote statistical significance as determined by unpaired t-test.

FIG. 14 shows expression of SARS-CoV-2 Spike immunogen in the serum of cynomolgus monkeys after having been administered a 100 μg dose of lipid nanoparticle (LNP)-formulated circular RNA via intramuscular injection at day 0 (prime) and day 28 (boost).

FIG. 15 shows expression of a SARS-CoV-2 RBD immunogen fused to a T4 foldon multimerization domain in the serum of cynomolgus monkeys after having been administered a 100 μg dose of LNP-formulated circular RNA or a 1000 μg dose of adjuvanted circular RNA via intramuscular injection.

FIG. 16A shows that Spike-specific binding antibodies were primed in cynomolgus monkeys at day 42 after administration of the initial dose of LNP-formulated or adjuvanted circular RNA encoding either a SARS-CoV-2 Spike immunogen or a SARS-CoV-2 RBD immunogen fused to a T4 foldon multimerization domain.

FIG. 16B shows that RBD-specific binding antibodies were primed in cynomolgus monkeys at day 42 after administration of the initial dose of LNP-formulated or adjuvanted circular polyribonucleotide encoding either a SARS-CoV-2 Spike immunogen or SARS-CoV-2 RBD immunogen fused to a T4 foldon multimerization domain.

FIG. 17A shows that SARS-CoV-2 neutralizing antibodies were primed in cynomolgus monkeys at day 42 after administration of an initial 30 μg or 100 μg dose of LNP-formulated circular RNA encoding a SARS-CoV-2 Spike immunogen.

FIG. 17B shows that SARS-CoV-2 neutralizing antibodies were primed in cynomolgus monkeys at day 42 after administration of an initial dose of either a LNP-formulated circular polyribonucleotide encoding a SARS-CoV-2 RBD immunogen fused to a T4 foldon multimerization domain or an adjuvanted circular polyribonucleotide encoding a SARS-CoV-2 RBD immunogen fused to a T4 foldon multimerization domain.

### DETAILED DESCRIPTION

This disclosure provides compositions, pharmaceutical preparations, and methods relating to polyribonucleotides (e.g., circular polyribonucleotides or linear polyribonucleotides) encoding one or more immunogens and/or epitopes from a coronavirus. This disclosure also provides methods of using the circular polyribonucleotides encoding one or more one or more immunogens and/or epitopes from a coronavirus. Compositions and pharmaceutical preparations of circular polyribonucleotides described herein may induce an immune response in a subject upon administration. Compositions and pharmaceutical preparations of circular polyribonucleotides described herein may be used to treat or prevent a disease, disorder, or condition in a subject (e.g., SARS-CoV, e.g., SARS-CoV-1 or SARS-CoV-2).

### Circular Polyribonucleotide

The circular polyribonucleotides as disclosed herein comprise one or more expression sequences encoding one or more immunogens and/or epitopes from a coronavirus. This circular polyribonucleotide expresses the sequence encoding the one or more immunogens and/or epitopes from the coronavirus in a subject. In some embodiments, circular polyribonucleotides comprising one or more coronavirus immunogens and/or epitopes are used to produce an immune response in a subject. In some embodiments, circular polyribonucleotides comprising one or more coronavirus immunogens and/or epitopes are used to produce polyclonal antibodies as described herein.

### Coronavirus Immunogens and Epitopes

Circular polyribonucleotides described herein include at least one expression sequence encoding a coronavirus immunogen and/or epitope. Circular polyribonucleotides described herein may include multiple expression sequences, wherein at least one expression sequence encodes a coronavirus immunogen and/or epitope. Circular polyribonucleotides described herein may include two or more (two, three, four, five, six or more) expression sequences, wherein each expression sequence encodes a coronavirus immunogen and/or epitope. Circular polyribonucleotides described herein may include a first expression sequence that encodes a coronavirus immunogen and/or epitope and a second expression sequence that encodes an adjuvant. Circular polyribonucleotides described herein may include an expression sequence that encodes a coronavirus immunogen and/or epitope and a non-coding sequence that stimulates the innate immune system.

In some embodiments, the coronavirus is a pathogenic coronavirus. In some embodiments, the coronavirus is a respiratory pathogen. In some embodiments, the coronavirus is a blood borne pathogen. In some embodiments, the coronavirus is an enteric pathogen.

Non-limiting examples of coronaviruses of the disclosure include severe acute respiratory syndrome associated coronavirus (SARS-CoV, e.g., SARS-CoV-1, SARS-CoV-2), Middle East respiratory syndrome coronavirus (MERS-CoV), bat coronaviruses, zoonotic coronaviruses that can infect humans or other animals, newly emerged or newly discovered coronaviruses, and other coronaviruses.

In some embodiments, a circular polyribonucleotide comprises severe acute respiratory syndrome associated coronavirus (SARS-CoV) immunogens and/or epitopes. In some embodiments, a circular polyribonucleotide comprises SARS-CoV-1 immunogens and/or epitopes. In some embodiments, a circular polyribonucleotide comprises SARS-CoV-2 immunogens and/or epitopes. In some embodiments, a circular polyribonucleotide comprises Middle East respiratory syndrome coronavirus (MERS-CoV) immunogens and/or epitopes. In some embodiments, a circular polyribonucleotide comprises zoonotic coronavirus immunogens and/or epitopes that can infect humans or other animals. In some embodiments, a circular polyribonucleotide comprises immunogens and/or epitopes from a newly emerged coronavirus.

In some embodiments, a circular polyribonucleotide comprises Coronaviridae immunogens and/or epitopes.

In some embodiments, a circular polyribonucleotide comprises immunogens and/or epitopes from a genus or subgenus that is Alphacoronavirus, Betacoronavirus, Gammacoronavirus, Deltacoronavirus, Merbecovirus, or Sarbecovirus. In some embodiments, a circular polyribonucleotide comprises Betacoronavirus immunogens and/or epitopes. In some embodiments, a circular polyribonucleotide comprises Sarbecovirus immunogens and/or epitopes. In some embodiments, a circular polyribonucleotide comprises Merbecovirus immunogens and/or epitopes.

In some embodiments, the circular polyribonucleotide comprises immunogens and/or epitopes from a genus or subgenus of the omicron coronavirus variant (B.1.1.529. In some embodiments, the omicron coronavirus variant may be of the sublineage of BA.2, BA.2.75, BA.4.1, BA.4.1.8, BA.4.6.1, BA.4.6.4, BA.5, BA.5.1, BA.5.1.12, BA.5.1.25, BA.5.10.1, BA.5.2, BA.5.2.1, BA.5.2.6, BA.5.3, BA.5.3.1, BA.5.3.5, BA. 5.5., BA 5.6, BA.5.6.1, BA.5.7, BE.1.1, BF.10, BF.16, BF.31, BF.31.1, BF.7, BQ.1, BQ.1.1, BQ.1.8, XBB, or XBB.1.

In some embodiments, a circular polyribonucleotide comprises a sequence for an immunogen from a coronavirus that is a biosafety level 2 (BSL-2) pathogen). In some embodiments, a circular polyribonucleotide comprises a sequence from a coronavirus that is a biosafety level 3 (BSL-3) pathogen. In some embodiments, the coronavirus is a biosafety level 4 pathogen (BSL-4). In some embodiments, no approved drugs (e.g., antiviral or antibiotic drugs) are available to treat infection with the coronavirus from which the immunogen expressed by the circular polyribonucleotide is derived. In some embodiments, no approved vaccines are available to prevent or reduce the risk of infection with the coronavirus from which the immunogen expressed by the circular polyribonucleotide is derived.

An immunogen and/or epitope can be from a coronavirus surface protein, a coronavirus membrane protein, a coronavirus envelope protein, a coronavirus capsid protein, a coronavirus nucleocapsid protein, a coronavirus spike protein, a coronavirus receptor binding domain (RBD) of a spike protein, a coronavirus entry protein, a coronavirus membrane fusion protein, a coronavirus structural protein, a coronavirus non-structural protein, a coronavirus regulatory protein, a coronavirus accessory protein, a secreted coronavirus protein, a coronavirus polymerase protein, a coronavirus RNA polymerase, a coronavirus protease, a coronavirus glycoprotein, a coronavirus fusogen, a coronavirus helical capsid protein, a coronavirus icosahedral capsid protein, a coronavirus matrix protein, a coronavirus replicase, a coronavirus transcription factor, or a coronavirus enzyme.

Immunogens and/or epitopes from any number of coronaviruses are expressed by the circular polyribonucleotide. In some cases, the immunogens and/or epitopes are associated with or expressed by one coronavirus disclosed herein. In some embodiments, the immunogens and/or epitopes are associated with or expressed by two or more coronaviruses disclosed herein.

In some cases, two or more coronaviruses are phenotypically related. For example, compositions and methods of the disclosure can utilize immunogens and/or epitopes from two or more coronaviruses that are respiratory pathogens, two or more coronaviruses that are associated with severe disease, two or more coronaviruses that are associated with adverse outcomes in immunocompromised subjects (e.g., subjects for immunization), two or more coronaviruses that are associated with acute respiratory distress syndrome (ARDS), two or more coronaviruses that are associated with severe acute respiratory syndrome (SARS), two or more coronaviruses that are associated with middle eastern respiratory syndrome (MERS), or a combination thereof.

A circular polyribonucleotide can comprise or encode, for example, immunogens and/or epitopes from at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, or more coronaviruses. In some embodiments, the circular polyribonucleotide includes or encodes for immunogens and/or epitopes from at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, or more targets other than a coronavirus (e.g., a virus other than a coronavirus, such as an influenza virus).

In some embodiments, a circular polyribonucleotide comprises or encodes immunogens and/or epitopes from at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, or less coronaviruses. In some embodiments, the circular polyribonucleotide includes or encodes for immunogens and/or epitopes from at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, or less targets other than a coronavirus (e.g., a virus other than a coronavirus, such as an influenza virus).

In some embodiments, a circular polyribonucleotide comprises or encodes immunogens and/or epitopes from about 1, 2, 3, 4, 5, 6, 7, 8, 9, 10, 15, 20, 25, 30, 40, 50, 60, 70, 80, 90, or 100, coronaviruses. In some embodiments, a circular polyribonucleotide comprises or encodes immunogens and/or epitopes from about 1, 2, 3, 4, 5, 6, 7, 8, 9, 10, 15, 20, 25, 30, 40, 50, 60, 70, 80, 90, or 100, targets other than a coronavirus (e.g., a virus other than a coronavirus, such as an influenza virus).

In some embodiments, an immunogen and/or epitope is from a coronavirus, for example, a severe acute respiratory syndrome associated coronavirus (SARS-CoV, e.g., SARS-CoV-1, SARS-CoV-2), a Middle East respiratory syndrome coronavirus (MERS-CoV), or another coronavirus. In some embodiments, an immunogen and/or epitope of the disclosure is from a predicted open reading frame from a coronavirus genome.

New SARS isolates may be identified by a percent homology of 99%, 98%, 97%, 95%, 92%, 90%, 85%, or 80% homology of the polynucleotide sequence for specific genomic regions for the new virus with the polynucleotide sequence for specific genomic regions of the known SARS viruses. Additionally, new SARS isolates may be identified by a percent homology of 99%, 98%, 97%, 95%, 92%, 90%, 85%, or 80% homology of the polypeptide sequence encoded by the polynucleotide of specific genomic regions of the new SARS virus to the polypeptide sequence encoded by the polynucleotides of specific regions of the known SARS virus. These genomic regions may include regions (e.g., gene products or ORFs) which are typically in common among numerous coronaviruses, as well as group specific regions (e.g., immunogenic groups), such as, for example, any one of the following genomic regions which could be readily identified by a virologist skilled in the art: 5′ untranslated region (UTR), leader sequence, ORF1a, ORF1 b, nonstructural protein 2 (NS2), hemagglutinin-esterase glycoprotein (HE) (also referred to as E3), spike glycoprotein (S) (also referred to as E2), ORF3a, ORF3b, nonstructural protein 4 (NS4), envelope (small membrane) protein (E) (also referred to as sM), membrane glycoprotein (M) (also referred to as E1), ORF5a, ORF5b, nucleocapsid phosphoprotein (N), ORF6, ORF7a, ORF7b, ORF8, ORF8a, ORF8b, ORF9a, ORF9b, ORF10, intergenic sequences, receptor binding domain (RBD) of a spike protein, 3′UTR, or RNA dependent RNA polymerase (pol). The SARS virus may have identifiable genomic regions with one or more the above-identified genomic regions. A SARS viral immunogen includes a protein encoded by any one of these genomic regions. A SARS viral immunogen may be a protein or a fragment thereof, which is highly conserved with coronaviruses. A SARS viral immunogen may be a protein or fragment thereof, which is specific to the SARS virus (as compared to known coronaviruses).

In some embodiments, an immunogen and/or epitope of the disclosure is from a predicted transcript from a SARS-CoV genome. In some embodiments, an immunogen and/or epitope of the disclosure is from a protein encoded by an open reading frame from a SARS-CoV genome. Non-limiting examples of open reading frames in SARS-CoV genomes can include ORF1a, ORF1 b, spike (S), ORF3a, ORF3b, envelope (E), membrane (M), ORF6, ORF7a, ORF7b, ORF8, ORF8a, ORF8b, ORF9a, ORF9b, nucleocapsid (N), and ORF10.

ORF1a and ORF1 b encode 16 non-structural proteins (nsp), for example, nsp1, nsp2, nsp3, nsp4, nsp5, nsp6, nsp7, nsp8, nsp9, nsp10, nsp11, nsp12, nsp13, nsp14, nsp15, and nsp16. Nonstructural proteins, for example, contribute to viral replication, viral assembly, immune response modulation, or a combination thereof. In some embodiments, the immunogen is a non-structural protein or is an immunogenic sequence encoding a non-structural protein. In some embodiments, epitopes are from a coronavirus non-structural protein.

Spike (S) encodes a spike protein, which in some embodiments contributes to binding to a host cell receptor, fusion of the virus with the host cell membrane, entry of the virus into a host cell, or a combination thereof. Spike protein can be an immunogen. In some embodiments, epitopes of the disclosure are from a spike protein. In some embodiments, epitopes of the disclosure comprise a receptor binding domain of a Spike protein. In some embodiments, epitopes of the disclosure comprise an ACE2 binding domain of a Spike protein.

Envelope (E) encodes envelope protein, which in some embodiments contributes to virus assembly and morphogenesis. Envelope protein can be an immunogen. In some embodiments, epitopes of the disclosure are from a coronavirus envelope protein.

Membrane (M) encodes membrane protein, which in some embodiments contributes to viral assembly. Membrane protein can be an immunogen. In some embodiments, epitopes of the disclosure are from a coronavirus membrane protein.

Nucleocapsid (N) encodes nucleocapsid protein, which in some embodiments can form complexes with genomic RNA and contribute to viral assembly, and/or interact with M protein.

Nucleocapsid protein can be an immunogen. In some embodiments, epitopes of the disclosure are from a coronavirus nucleocapsid protein.

ORF3a, ORF3b, ORF6, ORF7a, ORF7b, ORF8, ORF8a, ORF8b, ORF9a, ORF9b, and ORF10 encodes accessory proteins. In some embodiments, accessory proteins can modulate host cell signaling, modulate host cell immune responses, be incorporated into mature virions as minor structural proteins, or a combination thereof. An accessory protein can be an immunogen. In some embodiments, epitopes of the disclosure are from a coronavirus accessory protein.

Compositions and methods of the disclosure can utilize immunogens and/or epitopes that are encoded by or derived from one or more open reading frames of a SARS-CoV genome. For example, immunogens and/or epitopes can be encoded by or derived from ORF a, ORF b, spike (S), ORF3a, ORF3b, envelope (E), membrane (M), ORF6, ORF7a, ORF7b, ORF8, ORF8a, ORF8b, ORF9a, ORF9b, nucleocapsid (N), ORF10, or any combination thereof.

In some embodiments, epitopes of the disclosure are from a spike protein. In some embodiments, the epitopes of the disclosure are from the omicron coronavirus spike protein. The omicron coronavirus spike protein has an amino acid sequence of SEQ ID NO: 283. In some embodiments, epitopes of the disclosure comprise a receptor binding domain (RBD) of a Spike protein. In some embodiments, epitopes of the disclosure comprise an ACE2 binding domain of a Spike protein. In some embodiments, epitopes of the disclosure comprise an S1 subunit Spike protein, an S2 subunit of spike protein, or a combination thereof. In some embodiments, epitopes of the disclosure comprise an ectodomain of a spike protein. In some embodiments, an epitope of the disclosure comprises Gln498, Thr500, Asn501, or a combination thereof from a coronavirus spike protein. In some embodiments, an epitope of the disclosure comprises Lys417, Tyr453, or a combination thereof from a coronavirus spike protein. In some embodiments, an epitope of the disclosure comprises Gln474, Phe486, or a combination thereof from a coronavirus spike protein. In some embodiments, an epitope of the disclosure comprises Gln498, Thr500, Asn501, Lys417, Tyr453, Gln474, Phe486, one or more equivalent amino acids from a spike protein variant or derivative, or a combination thereof from a coronavirus spike protein. In some embodiments, the spike protein of the disclosure comprises a D614G mutation, namely having an amino acid glycine (G) at the 614 position instead of aspartic acid (D). In some embodiments, an epitope of the disclosure comprises Gly614 from a spike protein variant or derivative, or combination thereof from a coronavirus spike protein. In some cases, the D614G mutation can lead to reduction of S1 shedding and increase in the infectivity of the coronavirus. In some embodiments, the spike protein of the disclosure comprises may include one or more of a T19I, L24del, P25del, P26del, A27S, H69del, V70del, V213G, G229D, R346T, S371F, S373P, S375F, T376A, D405N, R408S, K417N, N440K, K444T, L452R, N460K, S477N, T478K, E484A, F486V, Q498R, N501Y, Y505H, D614G, H655Y, N679K, P681H, N764K, D796Y, Q954H, N969K, Y144del, P251 L, and S256 L mutations in comparison to the wildtype spike protein. In some embodiments, the spike protein of the disclosure comprises may include the mutations T19I, L24del, P25del, P26del, A27S, H69del, V70del, V213G, G229D, R346T, S371F, S373P, S375F, T376A, D405N, R408S, K417N, N440K, K444T, L452R, N460K, S477N, T478K, E484A, F486V, Q498R, N501Y, Y505H, D614G, H655Y, N679K, P681H, N764K, D796Y, Q954H, N969K, Y144del, P251 L, and S256 L in comparison to the wildtype spike protein. In some embodiments, the spike protein of the disclosure comprises may include one or more of a T19I, L24del, P25del, P26del, A27S, G142D, K147E, W152R, F157 L, I210V, V213G, G257S, G339H, R346T, K356T, S371F, S373P, T376A, D405N, R408S, K417N, N440K, G446S, N460K, S477N, T478K, E484A, F486S, F490S, Q498R, N501Y, Y505H, D574V, D614G, H655Y, N679K, P681H, N764K, D796Y, Q954H, N969K, D1199N, M177T, N185D, N211del, L212I, K444T, N450D, L452R, F486P, F4861, S494P, and H1101Y mutations in comparison to the wildtype spike protein. In some embodiments, the spike protein of the disclosure comprises may include the mutations T19I, L24del, P25del, P26del, A27S, G142D, K147E, W152R, F157 L, 1210V, V213G, G257S, G339H, R346T, K356T, S371F, S373P, T376A, D405N, R408S, K417N, N440K, G446S, N460K, S477N, T478K, E484A, F486S, F490S, Q498R, N501Y, Y505H, D574V, D614G, H655Y, N679K, P681H, N764K, D796Y, Q954H, N969K, D1199N, M177T, N185D, N211del, L212I, K444T, N450D, L452R, F486P, F4861, S494P, and H1101Y in comparison to the wildtype spike protein.

In some embodiments, the spike protein of the disclosure comprises may include one or more of a T19I, L24del, P25del, P26del, A27S, H69del, V70del, G142D, Y144del, V213G, G339D, S371F, S373P, S375F, T376A, D405N, R408S, K417N, N440K, K444T, L452R, N460K, S477N, T478K, E484A, F486V, Q498R, N501Y, Y505H, D614G, H655Y, N679K, P681H, N764K, D796Y, Q954H, and N969K, and P251H mutations in comparison to the wildtype spike protein. In some embodiments, the spike protein of the disclosure comprises may include the mutations T19I, L24del, P25del, P26del, A27S, H69del, V70del, G142D, Y144del, V213G, G339D, S371F, S373P, S375F, T376A, D405N, R408S, K417N, N440K, K444T, L452R, N460K, S477N, T478K, E484A, F486V, Q498R, N501Y, Y505H, D614G, H655Y, N679K, P681H, N764K, D796Y, Q954H, and N969K in comparison to the wildtype spike protein.

In some embodiments, the spike protein of the disclosure comprises may include one or more of a T19I, L24del, P25del, P26del, A27S, V83A, G142D, Y144del, H146Q, Q183E, V213E, G252V, G339H, R346T, L368I, S371F, S373P, S375F, T376A, D405N, R408S, K417N, N440K, V445P, G446S, N460K, S477N, T478K, E484A, F486S, F490S, Q498R, N501Y, Y505H, D614G, H655Y, N679K, P681H, N764K, D796Y, Q954H, N969K, and H146K mutations in comparison to the wildtype spike protein. In some embodiments, the spike protein of the disclosure comprises may include the mutations T19I, L24del, P25del, P26del, A27S, V83A, G142D, Y144del, H146Q, Q183E, V213E, G252V, G339H, R346T, L368I, S371F, S373P, S375F, T376A, D405N, R408S, K417N, N440K, V445P, G446S, N460K, S477N, T478K, E484A, F486S, F490S, Q498R, N501Y, Y505H, D614G, H655Y, N679K, P681H, N764K, D796Y, Q954H, N969K, and H146K in comparison to the wildtype spike protein.

In some embodiments, immunogens and/or epitopes are encoded by or derived from ORF a. In some embodiments, immunogens and/or epitopes are encoded by or derived from a SARS-CoV ORF b. In some embodiments, immunogens and/or epitopes are encoded by or derived from a SARS-CoV spike. In some embodiments, immunogens and/or epitopes are encoded by or derived from a SARS-CoV ORF3a. In some embodiments, immunogens and/or epitopes are encoded by or derived from a SARS-CoV ORF3b. In some embodiments, immunogens and/or epitopes are encoded by or derived from a SARS-CoV envelope (E). In some embodiments, immunogens and/or epitopes are encoded by or derived from a SARS-CoV membrane (M). In some embodiments, immunogens and/or epitopes are encoded by or derived from a SARS-CoV ORF6. In some embodiments, immunogens and/or epitopes are encoded by or derived from a SARS-CoV ORF7a. In some embodiments, immunogens and/or epitopes are encoded by or derived from a SARS-CoV ORF7b. In some embodiments, immunogens and/or epitopes are encoded by or derived from a SARS-CoV ORF8. In some embodiments, immunogens and/or epitopes are encoded by or derived from a SARS-CoV ORF8a. In some embodiments, immunogens and/or epitopes are encoded by or derived from a SARS-CoV ORF9a. In some embodiments, immunogens and/or epitopes are encoded by or derived from a SARS-CoV ORF9b. In some embodiments, immunogens and/or epitopes are encoded by or derived from a SARS-CoV nucleocapsid (N). In some embodiments, immunogens and/or epitopes are encoded by or derived from a SARS-CoV ORF10. In some embodiments, immunogens and/or epitopes are encoded by or derived from a SARS-CoV spike (S), envelope (E), membrane (M), and nucleocapsid (N).

In some embodiments, immunogens and/or epitopes are not encoded by or derived from a SARS-CoV ORF a. In some embodiments, immunogens and/or epitopes are not encoded by or derived from a SARS-CoV ORF b. In some embodiments, immunogens and/or epitopes are not encoded by or derived from a SARS-CoV spike. In some embodiments, immunogens and/or epitopes are not encoded by or derived from a SARS-CoV ORF3a. In some embodiments, immunogens and/or epitopes are not encoded by or derived from a SARS-CoV ORF3b. In some embodiments, immunogens and/or epitopes are not encoded by or derived from a SARS-CoV envelope (E). In some embodiments, immunogens and/or epitopes are not encoded by or derived from a SARS-CoV membrane (M). In some embodiments, immunogens and/or epitopes are not encoded by or derived from a SARS-CoV ORF6. In some embodiments, immunogens and/or epitopes are not encoded by or derived from a SARS-CoV ORF7a. In some embodiments, immunogens and/or epitopes are not encoded by or derived from a SARS-CoV ORF7b. In some embodiments, immunogens and/or epitopes are not encoded by or derived from a SARS-CoV ORF8. In some embodiments, immunogens and/or epitopes are not encoded by or derived from a SARS-CoV ORF8a. In some embodiments, immunogens and/or epitopes are not encoded by or derived from a SARS-CoV ORF9a. In some embodiments, immunogens and/or epitopes are not encoded by or derived from a SARS-CoV ORF9b. In some embodiments, immunogens and/or epitopes are not encoded by or derived from a SARS-CoV nucleocapsid (N). In some embodiments, immunogens and/or epitopes are not encoded by or derived from a SARS-CoV ORF10. In some embodiments, immunogens and/or epitopes are not encoded by or derived from a SARS-CoV spike (S), envelope (E), membrane (M), and nucleocapsid (N).

An immunogen and/or epitope can be encoded by or derived from SARS-CoV2.

A non-limiting example of a SARS-CoV-2 genome is provided in DB Source accession MN908947.3, the complete genome sequence of a SARS-CoV2 isolate, the content of which is incorporated herein by reference in its entirety. DB Source accession MN908947.3: 21563-25384 corresponds to the S protein, the content of which is incorporated herein by reference in its entirety. A non-limiting example of a SARS-CoV-2 spike protein is provided in GenBank Sequence: QHD43416.1, the sequence of a spike protein of a Severe acute respiratory syndrome coronavirus 2 isolate, the content of which is incorporated herein by reference in its entirety A non-limiting example of a SARS-CoV-2 genome is provided in sequence NCBI Reference Sequence accession number NC_045512, version NC_045512.2, the complete genome sequence of SARS-CoV2 isolate Wuhan-Hu-1, the content of which is incorporated herein by reference in its entirety.

A non-limiting example of a SARS-CoV-2 genome is provided in sequence NCBI Reference Sequence accession number MW450666, the complete genome sequence of SARS-CoV2 isolate, the content of which is incorporated herein by reference in its entirety.

A non-limiting example of a SARS-CoV-2 genome is provided in sequence NCBI Reference Sequence accession number MW487270, the complete genome sequence of SARS-CoV2 lineage B.1.1.7 virus, the content of which is incorporated herein by reference in its entirety.

A non-limiting example of a SARS-CoV-2 genome is provided in sequence GISAID Reference Sequence accession number EPI_-SL_10894052-EPI_ISL_10894090, the complete genome sequence of severe acute respiratory syndrome coronavirus 2, the content of which is incorporated herein by reference in its entirety.

A non-limiting example of a SARS-CoV-2 genome is provided in sequence GISAID Reference Sequence accession number EPI_ISL_792683, the complete genome sequence of SARS-CoV2 lineage P.1 virus, the content of which is incorporated herein by reference in its entirety.

A non-limiting example of a SARS-CoV-2 genome is provided in sequence GISAID Reference Sequence accession number EPI_ISL_678615, the complete genome sequence of SARS-CoV2 lineage B.1.351 virus, the content of which is incorporated herein by reference in its entirety.

Non-limiting examples of a SARS-CoV-2 genome are provided in sequence NCBI Reference Sequence accession numbers MW972466-MW974550, the complete genome sequence of SARS-CoV2 lineage B.1.427 and B.1.429 virus, the contents of which are incorporated herein by reference in their entirety.

Non-limiting examples of a SARS-CoV-2 genome are provided in sequence NCBI Reference Sequence accession numbers MZ156756-MZ226428, the complete genome sequence of SARS-CoV2 virus, the contents of which are incorporated herein by reference in their entirety.

In some embodiments, the SAR-CoV-2 genome is provided in the GISAID Database at www.gisaid.org. In some embodiments, the SARS-CoV-2 genome is provided in the International Nucleotide Sequence Database Collaboration (INSDC) at www.insdc.org.

In some embodiments, an immunogen and/or epitope of the disclosure is from a predicted transcript from a SARS-CoV-2 genome. In some embodiments, an immunogen and/or epitope of the disclosure is from a protein encoded by an open reading frame from a SARS-CoV-2 genome, or a derivative thereof. Non-limiting examples of open reading frames in the SARS-CoV-2 genome include ORF1a, ORF1b, spike (S), ORF3a, envelope (E), membrane (M), ORF6, ORF7a, ORF7b, ORF8, nucleocapsid (N), and ORF10. In some embodiments, a SARS-Co V-2 genome encodes an ORF3b, ORF9a, ORF9b, or a combination thereof. In some embodiments, a SARS-CoV-2 genome does not encode an ORF3b, ORF9a, ORF9b, or any combination thereof.

Nonlimiting examples of amino acid sequences are provided in TABLE 1. In some embodiments, the immunogen comprises a sequence having at least about 80%, 85%, 90%, 95%, 97%, 98%, 99%, or 100% sequence identity to a sequence from TABLE 1.

TABLE 1
Examples of amino acid sequence of proteins encoded by a SARS-CoV-2 genome.

| SEQ ID | | |
|---|---|---|
| NO: | Description | Sequence |
| 1 | Spike (S) protein | MFVFLVLLPLVSSQCVNLTTRTQLPPAYTNSFTRGVYYPDKVFRSSV |
| | | LHSTQDLFLPFFSNVTWFHAIHVSGTNGTKRFDNPVLPFNDGVYFAS |
| | | TEKSNIIRGWIFGTTLDSKTQSLLIVNNATNVVIKVCEFQFCNDPFLGV |
| | | YYHKNNKSWMESEFRVYSSANNCTFEYVSQPFLMDLEGKQGNFKN |
| | | LREFVFKNIDGYFKIYSKHTPINLVRDLPQGFSALEPLVDLPIGINITRF |
| | | QTLLALHRSYLTPGDSSSGWTAGAAAYYVGYLQPRTFLLKYNENGTI |
| | | TDAVDCALDPLSETKCTLKSFTVEKGIYQTSNFRVQPTESIVRFPNIT |
| | | NLCPFGEVFNATRFASVYAWNRKRISNCVADYSVLYNSASFSTFKCY |
| | | GVSPTKLNDLCFTNVYADSFVIRGDEVRQIAPGQTGKIADYNYKLPD |
| | | DFTGCVIAWNSNNLDSKVGGNYNYLYRLFRKSNLKPFERDISTEIYQ |
| | | AGSTPCNGVEGFNCYFPLQSYGFQPTNGVGYQPYRVVVLSFELLHA |
| | | PATVCGPKKSTNLVKNKCVNFNFNGLTGTGVLTESNKKFLPFQQFG |
| | | RDIADTTDAVRDPQTLEILDITPCSFGGVSVITPGTNTSNQVAVLYQD |
| | | VNCTEVPVAIHADQLTPTWRVYSTGSNVFQTRAGCLIGAEHVNNSY |
| | | ECDIPIGAGICASYQTQTNSPRRARSVASQSIIAYTMSLGAENSVAYS |
| | | NNSIAIPTNFTISVTTEILPVSMTKTSVDCTMYICGDSTECSNLLLQYG |
| | | SFCTQLNRALTGIAVEQDKNTQEVFAQVKQIYKTPPIKDFGGFNFSQI |
| | | LPDPSKPSKRSFIEDLLFNKVTLADAGFIKQYGDCLGDIAARDLICAQK |
| | | FNGLTVLPPLLTDEMIAQYTSALLAGTITSGWTFGAGAALQIPFAMQM |
| | | AYRFNGIGVTQNVLYENQKLIANQFNSAIGKIQDSLSSTASALGKLQD |
| | | VVNQNAQALNTLVKQLSSNFGAISSVLNDILSRLDKVEAEVQIDRLITG |
| | | RLQSLQTYVTQQLIRAAEIRASANLAATKMSECVLGQSKRVDFCGKG |
| | | YHLMSFPQSAPHGVVFLHVTYVPAQEKNFTTAPAICHDGKAHFPRE |
| | | GVFVSNGTHWFVTQRNFYEPQIITTDNTFVSGNCDVVIGIVNNTVYD |
| | | PLQPELDSFKEELDKYFKNHTSPDVDLGDISGINASVVNIQKEIDRLN |
| | | EVAKNLNESLIDLQELGKYEQYIKWPWYIWLGFIAGLIAIVMVTIMLCC |
| | | MTSCCSCLKGCCSCGSCCKFDEDDSEPVLKGVKLHYT |
| 2 | Envelope (E) | MYSFVSEETGTLIVNSVLLFLAFVVFLLVTLAILTALRLCAYCCNIVNVS |
| | protein | LVKPSFYVYSRVKNLNSSRVPDLLV |
| 3 | Membrane (M) | MADSNGTITVEELKKLLEQWNLVIGFLFLTWICLLQFAYANRNRFLYII |
| | protein | KLIFLWLLWPVTLACFVLAAVYRINWITGGIAIAMACLVGLMWLSYFIA |
| | | SFRLFARTRSMWSFNPETNILLNVPLHGTILTRPLLESELVIGAVILRG |
| | | HLRIAGHHLGRCDIKDLPKEITVATSRTLSYYKLGASQRVAGDSGFAA |
| | | YSRYRIGNYKLNTDHSSSSDNIALLVQ |
| 4 | Nucleocapsid (N) | MSDNGPQNQRNAPRITFGGPSDSTGSNQNGERSGARSKQRRPQG |
| | protein | LPNNTASWFTALTQHGKEDLKFPRGQGVPINTNSSPDDQIGYYRRA |
| | | TRRIRGGDGKMKDLSPRWYFYYLGTGPEAGLPYGANKDGIIWVATE |
| | | GALNTPKDHIGTRNPANNAAIVLQLPQGTTLPKGFYAEGSRGGSQAS |
| | | SRSSSRSRNSSRNSTPGSSRGTSPARMAGNGGDAALALLLLDRLN |
| | | QLESKMSGKGQQQQGQTVTKKSAAEASKKPRQKRTATKAYNVTQA |
| | | FGRRGPEQTQGNFGDQELIRQGTDYKHWPQIAQFAPSASAFFGMS |
| | | RIGMEVTPSGTWLTYTGAIKLDDKDPNFKDQVILLNKHIDAYKTFPPT |
| | | EPKKDKKKKADETQALPQRQKKQQTVTLLPAADLDDFSKQLQQSMS |
| | | SADSTQA |
| 5 | ORF3a | MDLFMRIFTIGTVTLKQGEIKDATPSDFVRATATIPIQASLPFGWLIVG |
| | accessory protein | VALLAVFQSASKIITLKKRWQLALSKGVHFVCNLLLLFVTVYSHLLLVA |
| | | AGLEAPFLYLYALVYFLQSINFVRIIMRLWLCWKCRSKNPLLYDANYF |
| | | LCWHTNCYDYCIPYNSVTSSIVITSGDGTTSPISEHDYQIGGYTEKWE |
| | | SGVKDCVVLHSYFTSDYYQLYSTQLSTDTGVEHVTFFIYNKIVDEPEE |
| | | HVQIHTIDGSSGVVNPVMEPIYDEPTTTTSVPL |
| 6 | ORF6 accessory | MFHLVDFQVTIAEILLIIMRTFKVSIWNLDYIINLIIKNLSKSLTENKYSQL |
| | protein | DEEQPMEID |
| 7 | ORF7a | MKIILFLALITLATCELYHYQECVRGTTVLLKEPCSSGTYEGNSPFHPL |
| | accessory protein | ADNKFALTCFSTQFAFACPDGVKHVYQLRARSVSPKLFIRQEEVQEL |
| | | YSPIFLIVAAIVFITLCFTLKRKTE |
| 8 | ORF7b | MIELSLIDFYLCFLAFLLFLVLIMLIIFWFSLELQDHNETCHA |
| | accessory protein | |
| 9 | ORF8 accessory | MKFLVFLGIITTVAAFHQECSLQSCTQHQPYVVDDPCPIHFYSKWYIR |
| | protein | VGARKSAPLIELCVDEAGSKSPIQYIDIGNYTVSCLPFTINCQEPKLGS |
| | | LVVRCSFYEDFLEYHDVRVVLDFI |
| 10 | ORF10 | MGYINVFAFPFTIYSLLLCRMNSRNYIAQVDVVNFNLT |
| | accessory protein | |

Additional non-limiting examples of proteins encoded by a SARS-CoV-2 genome include those with the contents of NCBI accession numbers MT334522, MT334523, MT334524, MT334525, MT334526, MT334527, MT334528, MT334529, MT334530, MT334531, MT334532, MT334533, MT334534, MT334535, MT334536, MT334537, MT334538, MT334539, MT334540, MT334541, MT334542, MT334543, MT334544, MT334545, MT334546, MT334555, MT334547, MT334548, MT334549, MT334550, MT334551, MT334552, MT334553, MT334554, MT334556, MT334557, MT334558, MT334559, MT334560, MT334561, MT334562, MT334563, MT334564, MT334565, MT334566, MT334567, MT334568, MT334569, MT334570, MT334571, MT334572, MT334573, MT326097, MT326106, MT326107, MT326116, MT326117, MT326124, MT326125, MT326126, MT326127, MT326134, MT326135, MT326136, MT326137, MT326138, MT326139, MT326140, MT326141, MT326142, MT326143, MT326144, MT326145, MT326146, MT326148, MT326149, MT326150, MT326151, MT326152, MT326158, MT326159, MT326160, MT326161, MT326162, MT326168, MT326169, MT326170, MT326171, MT326172, MT326178, MT326179, MT326180, MT326181, MT326182, MT326183, MT326188, MT326189, MT326190, MT326191, MT326129, MT326121, MT326120, MT326119, MT326118, MT326111, MT326023, MT326025, MT326033, MT326035, MT326036, MT326040, MT326043, MT326045, MT326053, MT326055, MT326056, MT326063, MT326066, MT326070, MT326071, MT326072, MT326075, MT326076, MT326078, MT326079, MT326089, MT325563, MT325565, MT325566, MT326155, MT326163, MT326177, MT326130, MT326128, MT326110, MT326109, MT326108, MT326101, MT326100, MT326099, MT326098, MT326094, MT326093, MT326092, MT325568, MT325569, MT325590, MT325640, MT325606, MT325607, MT325608, MT325609, MT325610, MT325611, MT325616, MT325618, MT325619, MT325620, MT325622, MT325623, MT325624, MT325599, MT325600, MT325601, MT325602, MT325612, MT325613, MT325615, MT325617, MT325625, MT324062, MT324684, MT325573, MT325574, MT325577, MT325579, MT325586, MT325592, MT325593, MT325594, MT325598, MT325605, MT325626, MT325627, MT325633, MT325634, MT326028, MT326031, MT326091, MT326090, MT326085, MT326084, MT326083, MT326082, MT326081, MT326080, MT326077, MT326067, MT326057, MT326024, MT326026, MT326027, MT326032, MT326034, MT326037, MT326039, MT326041, MT326042, MT326044, MT326046, MT326047, MT326049, MT326050, MT326051, MT326052, MT326054, MT326059, MT326060, MT326061, MT326062, MT326064, MT326065, MT326068, MT326069, MT326073, MT326074, MT326088, MT327745, MT324679, MT325561, MT325571, MT325572, MT325575, MT325583, MT325587, MT325588, MT325589, MT325596, MT325597, MT325603, MT325604, MT325614, MT325621, MT325629, MT325630, MT325631, MT325632, MT325635, MT325636, MT325637, MT325638, MT325639, MT326086, MT326096, MT326102, MT326104, MT326105, MT326112, MT326113, MT326114, MT326115, MT326122, MT328034, MT325564, MT325567, MT326164, MT326165, MT326173, MT326174, MT326184, MT326185, MT326186, MT326187, MT325584, MT325585, MT326087, MT326095, MT326103, MT326123, MT326131, MT326132, MT326133, MT328033, MT325562, MT326147, MT326153, MT326154, MT326156, MT326157, MT326166, MT326167, MT326175, MT326176, MT324680, MT325570, MT325576, MT325578, MT325580, MT325581, MT325582, MT325591, MT325595, MT325628, MT326029, MT326030, MT326038, MT326048, MT326058, MT324681, MT324682, MT324683, MT328032, MT328035, MT322404, MT039874, MT322398, MT322409, MT322421, MT322423, MT322408, MT322413, MT322417, MT322394, MT322407, MT322418, MT322424, MT322411, MT077125, MT322395, MT322396, MT322397, MT322399, MT322400, MT322401, MT322402, MT322403, MT322405, MT322406, MT322414, MT322416, MT322419, MT322420, MT322410, MT322412, MT322415, MT322422, MT320538, MT320891, MT308692, MT308693, MT308695, MT308696, MT308698, MT308699, MT308701, MT308703, MT308704, MT308694, MT308697, MT308700, MT308702, MT293547, MT304476, MT304474, MT304475, MT304477, MT304478, MT304479, MT304481, MT304482, MT304484, MT304485, MT304486, MT304487, MT304488, MT304491, MT304480, MT304483, MT304489, MT304490, MT300186, MT292571, MT292576, MT292578, MT293186, MT292570, MT292573, MT293173, MT292575, MT293179, MT293180, MT293184, MT293189, MT293192, MT293193, MT293194, MT293201, MT293202, MT292572, MT292577, MT293185, MT293187, MT293188, MT291826, MT291832, MT291833, MT291835, MT291836, MT291831, MT293170, MT292574, MT293178, MT293181, MT293183, MT293195, MT293196, MT293197, MT293203, MT293204, MT293223, MT293212, MT293214, MT293215, MT293216, MT293219, MT293224, MT293225, MT293206, MT293208, MT293209, MT293221, MT295464, MT293160, MT293166, MT293171, MT293190, MT293161, MT293167, MT293168, MT293174, MT293175, MT293182, MT293191, MT293158, MT293162, MT293163, MT293164, MT293156, MT293157, MT293159, MT291834, MT291829, MT291827, MT291830, MT291828, MT293169, MT293200, MT293210, MT293211, MT293217, MT293218, MT295465, MT293198, MT293205, MT293207, MT293213, MT293220, MT293222, MT292581, MT292569, MT293172, MT293177, MT293176, MT293199, MT292580, MT292582, MT293165, MT292579, MT273658, MT281577, MT281530, MT276597, MT276598, MT276323, MT276328, MT276331, MT276329, MT276330, MT276324, MT276325, MT276327, MT276326, MT263388, MT263392, MT262900, MT262902, MT262906, MT262908, MT262912, MT262913, MT262914, MT262993, MT263074, MT263381, MT263391, MT262901, MT262903, MT262907, MT262909, MT262911, MT262899, MT262904, MT262915, MT262916, MT262897, MT262898, MT262905, MT262910, MT263400, MT263382, MT263383, MT263384, MT263385, MT262896, MT263407, MT263415, MT263406, MT263408, MT263422, MT263469, MT263439, MT263457, MT263459, MT263432, MT263450, MT263458, MT263467, MT263401, MT263411, MT263413, MT263426, MT263421, MT263443, MT263412, MT263416, MT263417, MT263423, MT263431, MT263461, MT263410, MT263424, MT263425, MT263427, MT263442, MT263402, MT263405, MT263409, MT263418, MT263419, MT263398, MT263399, MT263403, MT263404, MT263414, MT263430, MT263390, MT263434, MT263436, MT263446, MT263448, MT263452, MT263453, MT263456, MT263462, MT263463, MT263386, MT263387, MT263389, MT263428, MT263429, MT263433, MT263435, MT263437, MT263438, MT263440, MT263447, MT263449, MT263455, MT263444, MT263445, MT263451, MT263466, MT263420, MT263441, MT263454, MT263464, MT263465, MT263468, MT263460, MT263393, MT263394, MT263395, MT263396, MT263397, MT259226, MT259275, MT259276, MT259279, MT259247, MT258377, MT258378, MT258379, MT259231, MT259228, MT259238, MT259248, MT256917, MT259227, MT259236, MT256918, MT258380, MT259235, MT259237, MT259239, MT259281, MT259282, MT259283, MT259240, MT259243, MT259249, MT259250, MT259251, MT259256, MT259258, MT259266, MT259267, MT259274, MT259286, MT259287, MT259241, MT259242, MT258381, MT259257, MT259261, MT259262, MT259263, MT259264, MT259268, MT259269, MT259270, MT259271, MT259272, MT259273, MT259277, MT259278, MT259280, MT258383, MT258382, MT259246, MT256924, MT259244, MT259245, MT259252, MT259253, MT259254, MT259255, MT259259, MT259284, MT259229, MT259230, MT259265, MT259260, MT259285, LC534419, LC534418, MT253710, MT253709, MT253705, MT253708, MT253701, MT253702, MT253703, MT253704, MT253706, MT253707, MT251972, MT251974, MT251975, MT251973, MT251976, MT251979, MT253697, MT253699, MT253696, MT253698, MT253700, MT251977, MT251978, MT251980, MT246451, MT246461, MT246471, MT246472, MT246474, MT246483, MT246450, MT246453, MT246454, MT246462, MT246463, MT246464, MT246470, MT246473, MT246480, MT246484, MT246449, MT246455, MT246456, MT246478, MT246485, MT246488, MT246452, MT246460, MT246465, MT246481, MT246482, MT246490, MT246459, MT246468, MT246475, MT246477, MT246479, MT246457, MT246458, MT246466, MT246467, MT246469, MT246476, MT246486, MT246487, MT246489, MT233526, MT246667, MT240479, MT232870, MT232871, MT233523, MT232869, MT232872, MT233519, MT233521, MT233522, MT233520, MT226610, MT198653, MT198651, MT198652, MT192773, MT192758, MT192772, MT192765, MT192759, MT188341, MT188340, MT188339, MT186676, MT186681, MT186677, MT186678, MT187977, MT186680, MT186682, MT186679, MT184909, MT184911, MT184912, MT184913, MT184910, MT184907, MT184908, CADDYA000000000, MT163718, MT163719, MT163720, MT163714, MT163715, MT163721, MT163717, MT163737, MT163738, MT163712, MT163716, MT159706, MT159716, MT159719, MT159707, MT159717, MT159709, MT159715, MT159718, MT159722, MT159708, MT161607, MT159705, MT159710, MT159711, MT159712, MT159713, MT159714, MT159720, MT159721, MT121215, MT159778, MT066156, LC529905, MT050493, MT012098, MT152900, MT152824, MT135044, MT135042, MT135041, MT135043, MT126808, MT127113, MT127114, MT127116, MT127115, LC528232, LC528233, MT123293, MT123291, MT123290, MT123292, MT118835, MT111896, MT111895, MT106052, MT106053, MT106054, MT093571, MT093631, MT081061, MT081063, MT081066, MT081062, MT081064, MT081065, MT081067, MT081059, MT081060, MT081068, MT072667, MT072668, MT072688, MT066157, MT066176, MT066159, MT066175, MT066158, LC523809, LC523807, LC523808, MT044258, MT044257, MT050416, MT050417, MT042773, MT042774, MT042775, MT042776, MT049951, MT050414, MT050415, MT042777, MT042778, MT039887, MT039888, MT039890, MT039873, LC522350, MT027062, MT027063, MT027064, MT020881, MT019530, MT019531, MT019533, MT020880, MT019532, MT019529, MT020781, LR757995, LR757998, LR757996, LR757997, MT007544, MT008022, MT008023, MN996531, MN996530, MN996527, MN996528, MN996529, MN997409, MN988668, MN988669, MN994467, MN994468, MN988713, MN938384, MN975262, MN985325, MN938386, MN938388, MN938385, MN938387, MN938390, MN938389, MN975263, MN975267, MN975268, MN975265, MN975264, MN975266, MN970004, MN970003, MN908947, OL6728316.1 each of which is incorporated herein by reference in its entirety.

In particular embodiments, a circular polyribonucleotide comprises a SARS-CoV-2 immunogen described in TABLE 2. In some embodiments, the immunogen comprises a sequence having at least about 80%, 85%, 90%, 95%, 97%, 98%, 99%, or 100% sequence identity to a sequence from TABLE 2.

TABLE 2
Descriptions of constructs and SARS-CoV-2 ORFs

| | ORF (SEQ | ORF | Proline | Cloning | Circularization | |
|---|---|---|---|---|---|---|
| Construct | ID NO.) | Description | substitutions | optimization | optimization | IRES |
| p1 | 1 (13) | S protein | Yes | Yes | No | CVB3 |
| SEQ ID | | transmembrane | | | | |
| NO. 12 | | (TM) domain | | | | |
| | | completely | | | | |
| | | removed, and a | | | | |
| | | trimerization | | | | |
| | | domain added | | | | |
| p3 | 3 (15) | S protein | Yes | Yes | No | CVB3 |
| SEQ ID | | transmembrane | | | | |
| NO. 14 | | (TM) domain | | | | |
| | | fully intact | | | | |
| p5 | 5 (17) | S protein | Yes | Yes | No | CVB3 |
| SEQ ID | | transmembrane | | | | |
| NO. 16 | | (TM) domain | | | | |
| | | partially removed | | | | |
| | | (111 of 195 | | | | |
| | | nucleotides | | | | |
| | | were removed) | | | | |
| | | (nucleotides | | | | |
| | | 3709-3819 were | | | | |
| | | removed) | | | | |
| p7 | 7 (19) | S protein | Yes | Yes | Yes | CVB3 |
| SEQ ID | | transmembrane | | | | |
| NO. 18 | | (TM) domain | | | | |
| | | completely | | | | |
| | | removed, and a | | | | |
| | | trimerization | | | | |
| | | domain added | | | | |
| p9 | 9 (21) | S protein | Yes | Yes | Yes | CVB3 |
| SEQ ID | | transmembrane | | | | |
| NO. 20 | | (TM) domain | | | | |
| | | fully intact | | | | |
| p11 | 11 (23) | S protein | Yes | Yes | Yes | CVB3 |
| SEQ ID | | transmembrane | | | | |
| NO. 22 | | (TM) domain | | | | |
| | | partially removed | | | | |
| | | (111 of 195 | | | | |
| | | nucleotides | | | | |
| | | were removed) | | | | |
| | | (nucleotides | | | | |
| | | 3709-3819 were | | | | |
| | | removed) | | | | |
| p13 | 13 (25) | S protein | N/A | N/A | N/A | CVB3 |
| SEQ ID | | receptor binding | | | | |
| NO. 24 | | domain (RBD) | | | | |
| | | only with | | | | |
| | | secretion signal | | | | |
| | | translationally | | | | |
| | | fused to the 5′ | | | | |
| | | end | | | | |
| p15 | 3 (15) | S protein | Yes | Yes | N/A | EMCV |
| | | transmembrane | | | | |
| | | (TM) domain | | | | |
| | | completely | | | | |
| | | removed, and a | | | | |
| | | trimerization | | | | |
| | | domain added | | | | |
| p17 | 5 (17) | S protein | Yes | Yes | N/A | EMCV |
| | | transmembrane | | | | |
| | | (TM) domain | | | | |
| | | fully intact | | | | |
| p19 | 1 (13) | S protein | Yes | Yes | N/A | EMCV |
| | | transmembrane | | | | |
| | | (TM) domain | | | | |
| | | partially | | | | |
| | | removed (111 | | | | |
| | | of 195 | | | | |
| | | nucleotides | | | | |
| | | were removed) | | | | |
| | | (nucleotides | | | | |
| | | 3709-3819 were | | | | |
| | | removed) | | | | |
| p21 | 7 (19) | S protein | Yes | Yes | Yes | EMCV |
| | | transmembrane | | | | |
| | | (TM) domain | | | | |
| | | completely | | | | |
| | | removed, and a | | | | |
| | | trimerization | | | | |
| | | domain added | | | | |
| p23 | 9 (21) | S protein | Yes | Yes | Yes | EMCV |
| | | transmembrane | | | | |
| | | (TM) domain | | | | |
| | | fully intact | | | | |
| p25 | 11 (23) | S protein | Yes | Yes | Yes | EMCV |
| | | transmembrane | | | | |
| | | (TM) domain | | | | |
| | | partially removed | | | | |
| | | (111 of 195 | | | | |
| | | nucleotides | | | | |
| | | were removed) | | | | |
| | | (nucleotides | | | | |
| | | 3709-3819 were | | | | |
| | | removed) | | | | |
| p27 | 13 (25) | S protein | N/A | N/A | N/A | EMCV |
| | | receptor binding | | | | |
| | | domain (RBD) | | | | |
| | | only with | | | | |
| | | secretion signal | | | | |
| | | translationally | | | | |
| | | fused to the 5′ end | | | | |
| p33 | 33 (26) | S protein | N/A | N/A | N/A | EMCV |
| | | receptor binding | | | | |
| | | domain (RBD) | | | | |
| | | only with | | | | |
| | | secretion signal | | | | |
| | | translationally | | | | |
| | | fused to the 5′ | | | | |
| | | end and RBD | | | | |
| | | type II terminator | | | | |
| | | removed | | | | |
| p35 | 35 (27) | S protein | Yes | Yes | No | EMCV |
| | | transmembrane | | | | |
| | | (TM) domain | | | | |
| | | fully intact and | | | | |
| | | RBD type II | | | | |
| | | terminator removed | | | | |
| p36 | 36 (28) | S protein | Yes | Yes | Yes | EMCV |
| | | transmembrane | | | | |
| | | (TM) domain | | | | |
| | | fully intact and | | | | |
| | | RBD type II | | | | |
| | | terminator removed | | | | |
| p39 | 39 (29) | S protein | N/A | N/A | N/A | EMCV |
| | | transmembrane | | | | |
| | | (TM) domain | | | | |
| | | fully intact. GC | | | | |
| | | optimized | | | | |
| p41 | 41 (30) | S protein | N/A | N/A | N/A | EMCV |
| | | receptor binding | | | | |
| | | domain (RBD) | | | | |
| | | only with | | | | |
| | | secretion signal | | | | |
| | | translationally | | | | |
| | | fused to the 5′ | | | | |
| | | end and RBD | | | | |
| | | type II terminator | | | | |
| | | removed. GC | | | | |
| | | optimized | | | | |
| p44 | 44 (48) | S protein | N/A | N/A | N/A | EMCV |
| | | receptor binding | | | | |
| | | domain (RBD) | | | | |
| | | only with IL-2 | | | | |
| | | secretion signal | | | | |
| | | translationally | | | | |
| | | fused to the 5′ | | | | |
| | | end and RBD type | | | | |
| | | II terminator | | | | |
| | | removed | | | | |
| p45 | 45 (49) | S protein | N/A | N/A | N/A | EMCV |
| | | receptor binding | | | | |
| | | domain (RBD) | | | | |
| | | only with Gluc | | | | |
| | | secretion signal | | | | |
| | | translationally | | | | |
| | | fused to the 5′ | | | | |
| | | end and RBD | | | | |
| | | type II | | | | |
| | | terminator | | | | |
| | | removed | | | | |

In TABLE 2, “proline substitutions” denote proline substitutions at residues 986 and 987, as well as a “GSAS” substitution (SEQ ID NO: 336) at the furin cleavage site (residues 682-685). For “cloning optimization,” single base substitution was made at coordinate 2541 to destroy a Bsal site to assist in Golden Gate Cloning construction of the plasmid DNA template. For “circularization optimizations”: four single nucleotides—at positions 2307, 2790, 159 and 315—were substituted to destroy sites that could potentially bind circularization elements of splint nucleic acid sequences, thereby potentially inhibiting efficient ligation. For constructs that have type II terminator removed (e.g., p33, p35, p36, p39, p41, p44, and p45): two single nucleotides—at positions 1047, 1049 were substituted to destroy type II terminator site. For constructs that have GO optimization (e.g., p39 and p41), GO optimization was performed such that GO content was approximately 50%. All single base pair substitutions were designed to be translationally silent. Further, in TABLE 2, IRES is EMCV (SEQ ID NO: 31) or is CV1B3 (SEQ ID NO: 45).

In some embodiments, the circular polyribonucleotide includes an open reading frame encoding a SARS-CoV-2 immunogen having an amino acid sequence that is at least about 80% (e.g., about 81%, 82%, 83%, 84%, 85%, 86%, 87%, 88%, 89%, 90%, 91%, 92%, 93%, 94%, 95%, 96%, 97%, 98%, 99%, or 100%) identical to any one of SEQ ID NOs: 63-111 and 283-291. In some embodiments, the circular polyribonucleotide includes an open reading frame encoding a SARS-CoV-2 immunogen having an amino acid sequence that is at least about 90% (e.g., about 91%, 92%, 93%, 94%, 95%, 96%, 97%, 98%, 99%, or 100%) identical to any one of SEQ ID NOs: 63-111 and 283-291. In some embodiments, the circular polyribonucleotide includes an open reading frame encoding a SARS-CoV-2 immunogen having an amino acid sequence that is at least about 95% (e.g., about 96%, 97%, 98%, 99%, or 100%) identical to any one of SEQ ID NOs: 63-111 and 283-291. In some embodiments, the circular polyribonucleotide includes an open reading frame encoding a SARS-CoV-2 immunogen having an amino acid sequence that is any one of SEQ ID NOs: 63-111 and 283-291.

In some embodiments, the SARS-CoV-2 immunogen is an immunogenic fragment including a contiguous stretch of at least 100, 150, 200, 250, 300, 350, 400, 450, 500, 550, 600, 650, 700, 750, 800, 850, 900, 950, 1000, 1100, 1200, 1300, 1400, or 1500 amino acids of any one of SEQ ID NOs: 63-111 and 283-291. In some embodiments, the SARS-CoV-2 immunogen is an immunogenic fragment including a contiguous stretch of at least 50%, 60%, 70%, 80%, 90%, or 95% of the amino acids of a sequence any one of SEQ ID NOs: 63-111 and 283-291.

In particular embodiments, the circular polyribonucleotide includes an open reading frame with a nucleic acid sequence encoding a SARS-CoV-2 immunogen, wherein the nucleic acid sequence has at least about 80% (e.g., about 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 112-174 and 292-300. In some embodiments, the circular polyribonucleotide includes an open reading frame with a nucleic acid sequence encoding a SARS-CoV-2 immunogen, wherein the nucleic acid sequence has at least about 90% (e.g., about 91%, 92%, 93%, 94%, 95%, 96%, 97%, 98%, 99%, or 100%) sequence identity to any one of SEQ ID NOs: 112-174 and 292-300. In some embodiments, the circular polyribonucleotide includes an open reading frame with a nucleic acid sequence encoding a SARS-CoV-2 immunogen, wherein the nucleic acid sequence has at least about 95% (e.g., about 96%, 97%, 98%, 99%, or 100%) sequence identity to any one of SEQ ID NOs: 112-174 and 292-300. In certain embodiments, the circular polyribonucleotide includes an open reading frame with a nucleic acid sequence encoding a SARS-CoV-2 immunogen, wherein the nucleic acid sequence is any one of SEQ ID NOs: 112-174 and 292-300. In some embodiments, the polyribonucleotide sequence encoding the SARS-CoV-2 immunogen is a fragment including a contiguous stretch of at least 300, 400, 500, 600, 700, 800, 900, 1000, 1100, 1200, 1500, 2000, 2500, 3000, 3500, 4000, or 4500 nucleotides of any one of SEQ ID NOs: 112-174 and 292-300. In some embodiments, the polyribonucleotide sequence encoding the SARS-CoV-2 immunogen is a fragment including a contiguous stretch of at least 50%, 60%, 70%, 80%, 90%, or 95% of the amino acids of any one of SEQ ID NOs: 112-174 and 292-300.

In particular embodiments, the circular polyribonucleotide includes an open reading frame with a nucleic acid sequence encoding a SARS-CoV-2 immunogen, wherein the nucleic acid sequence has at least about 80% (e.g., about 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 219-281. In some embodiments, the circular polyribonucleotide includes an open reading frame with a nucleic acid sequence encoding a SARS-CoV-2 immunogen, wherein the nucleic acid sequence has at least about 90% (e.g., about 91%, 92%, 93%, 94%, 95%, 96%, 97%, 98%, 99%, or 100%) sequence identity to any one of SEQ ID NOs: 219-281. In some embodiments, the circular polyribonucleotide includes an open reading frame with a nucleic acid sequence encoding a SARS-CoV-2 immunogen, wherein the nucleic acid sequence has at least about 95% (e.g., about 96%, 97%, 98%, 99%, or 100%) sequence identity to any one of SEQ ID NOs: 219-281. In certain embodiments, the circular polyribonucleotide includes an open reading frame with a nucleic acid sequence encoding a SARS-CoV-2 immunogen, wherein the nucleic acid sequence is any one of SEQ ID NOs: 219-281. In some embodiments, the polyribonucleotide sequence encoding the SARS-CoV-2 immunogen is a fragment including a contiguous stretch of at least 300, 400, 500, 600, 700, 800, 900, 1000, 1100, 1200, 1500, 2000, 2500, 3000, 3500, 4000, or 4500 nucleotides of any one of SEQ ID NOs: 219-281. In some embodiments, the polyribonucleotide sequence encoding the SARS-CoV-2 immunogen is a fragment including a contiguous stretch of at least 50%, 60%, 70%, 80%, 90%, or 95% of the amino acids of any one of SEQ ID NOs: 219-281.

In particular embodiments, a circular polyribonucleotide comprises a SARS-CoV-2 RBD immunogen described in TABLE 3. In some embodiments, the immunogen comprises a sequence having at least about 80%, 85%, 90%, 95%, 97%, 98%, 99%, or 100% sequence identity to a sequence from TABLE 3. In some embodiments, the circular polyribonucleotide includes an open reading frame encoding a SARS-CoV-2 RBD immunogen having an amino acid sequence that is at least about 80% (e.g., about 81%, 82%, 83%, 84%, 85%, 86%, 87%, 88%, 89%, 90%, 91%, 92%, 93%, 94%, 95%, 96%, 97%, 98%, 99%, or 100%) identical to any one of SEQ ID NOs: 63-68, 74, 79, 81-86, and 98-111. In some embodiments, the circular polyribonucleotide includes an open reading frame encoding a SARS-CoV-2 RBD immunogen having an amino acid sequence that is at least about 90% (e.g., about 91%, 92%, 93%, 94%, 95%, 96%, 97%, 98%, 99%, or 100%) identical to any one of SEQ ID NOs: 63-68, 74, 79, 81-86, and 98-111. In some embodiments, the circular polyribonucleotide includes an open reading frame encoding a SARS-CoV-2 RBD immunogen having an amino acid sequence that is at least about 95% (e.g., about 96%, 97%, 98%, 99%, or 100%) identical to any one of SEQ ID NOs: 63-68, 74, 79, 81-86, and 98-111. In some embodiments, the circular polyribonucleotide includes an open reading frame encoding a SARS-CoV-2 immunogen having an amino acid sequence that is any one of SEQ ID NOs: 63-68, 74, 79, 81-86, and 98-111. In some embodiments, the SARS-CoV-2 RBD immunogen is an immunogenic fragment including a contiguous stretch of at least 100, 150, 200, 250, 300, 350, 400, 450, 500, 550, 600, 650, 700, 750, 800, 850, 900, 950, 1000, 1100, 1200, 1300, 1400, or 1500 amino acids of any one of SEQ ID NOs: 63-68, 74, 79, 81-86, and 98-111. In some embodiments, the SARS-CoV-2 RBD immunogen is an immunogenic fragment including a contiguous stretch of at least 50%, 60%, 70%, 80%, 90%, or 95% of the amino acids of a sequence any one of SEQ ID NOs: 63-68, 74, 79, 81-86, and 98-111.

In particular embodiments, the circular polyribonucleotide includes an open reading frame with a nucleic acid sequence encoding a SARS-CoV-2 RBD immunogen, wherein the nucleic acid sequence has at least about 80% (e.g., about 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 112-117, 123, 128, 133-138, and 163-174. In some embodiments, the circular polyribonucleotide includes an open reading frame with a nucleic acid sequence encoding a SARS-CoV-2 RBD immunogen, wherein the nucleic acid sequence has at least about 90% (e.g., about 91%, 92%, 93%, 94%, 95%, 96%, 97%, 98%, 99%, or 100%) sequence identity to any one of SEQ ID NOs: 112-117, 123, 128, 133-138, and 163-174. In some embodiments, the circular polyribonucleotide includes an open reading frame with a nucleic acid sequence encoding a SARS-CoV-2 RBD immunogen, wherein the nucleic acid sequence has at least about 95% (e.g., about 96%, 97%, 98%, 99%, or 100%) sequence identity to any one of SEQ ID NOs: 112-117, 123, 128, 133-138, and 163-174. In certain embodiments, the circular polyribonucleotide includes an open reading frame with a nucleic acid sequence encoding a SARS-CoV-2 RBD immunogen, wherein the nucleic acid sequence is any one of SEQ ID NOs: 112-117, 123, 128, 133-138, and 163-174. In some embodiments, the polyribonucleotide sequence encoding the SARS-CoV-2 RBD immunogen is a fragment including a contiguous stretch of at least 300, 400, 500, 600, 700, 800, 900, 1000, 1100, 1200, 1500, 2000, 2500, 3000, 3500, 4000, or 4500 nucleotides of any one of SEQ ID NOs: 112-117, 123, 128, 133-138, and 163-174. In some embodiments, the polyribonucleotide sequence encoding the SARS-CoV-2 RBD immunogen is a fragment including a contiguous stretch of at least 50%, 60%, 70%, 80%, 90%, or 95% of the amino acids of any one of SEQ ID NOs: 112-117, 123, 128, 133-138, and 163-174.

In particular embodiments, a circular polyribonucleotide comprises more than one SARS-CoV-2 RBD as described in TABLE 5. In some embodiments, the circular polyribonucleotide includes the open reading frames described in TABLE 5.

In particular embodiments, a circular polyribonucleotide comprises a SARS-CoV-2 Spike immunogen described in TABLE 4. In some embodiments, the immunogen comprises a sequence having at least about 80%, 85%, 90%, 95%, 97%, 98%, 99%, or 100% sequence identity to a sequence from TABLE 4. In some embodiments, the circular polyribonucleotide includes an open reading frame encoding a SARS-CoV-2 Spike immunogen having an amino acid sequence that is at least about 80% (e.g., about 81%, 82%, 83%, 84%, 85%, 86%, 87%, 88%, 89%, 90%, 91%, 92%, 93%, 94%, 95%, 96%, 97%, 98%, 99%, or 100%) identical to any one of SEQ ID NOs: 69-73, 75-78, 80, 87-97, and 283-286. In some embodiments, the circular polyribonucleotide includes an open reading frame encoding a SARS-CoV-2 Spike immunogen having an amino acid sequence that is at least about 90% (e.g., about 91%, 92%, 93%, 94%, 95%, 96%, 97%, 98%, 99%, or 100%) identical to any one of SEQ ID NOs: 69-73, 75-78, 80, 87-97, and 283-286. In some embodiments, the circular polyribonucleotide includes an open reading frame encoding a SARS-CoV-2 Spike immunogen having an amino acid sequence that is at least about 95% (e.g., about 96%, 97%, 98%, 99%, or 100%) identical to any one of SEQ ID NOs: 69-73, 75-78, 80, 87-97, and 283-286. In some embodiments, the circular polyribonucleotide includes an open reading frame encoding a SARS-CoV-2 immunogen having an amino acid sequence that is any one of SEQ ID NOs: 69-73, 75-78, 80, 87-97, and 283-286. In some embodiments, the SARS-CoV-2 Spike immunogen is an immunogenic fragment including a contiguous stretch of at least 100, 150, 200, 250, 300, 350, 400, 450, 500, 550, 600, 650, 700, 750, 800, 850, 900, 950, 1000, 1100, 1200, 1300, 1400, or 1500 amino acids of any one of SEQ ID NOs: 69-73, 75-78, 80, 87-97, and 283-286. In some embodiments, the SARS-CoV-2 Spike immunogen is an immunogenic fragment including a contiguous stretch of at least 50%, 60%, 70%, 80%, 90%, or 95% of the amino acids of a sequence any one of SEQ ID NOs: 69-73, 75-78, 80, 87-97, and 283-286.

In particular embodiments, the circular polyribonucleotide includes an open reading frame with a nucleic acid sequence encoding a SARS-CoV-2 Spike immunogen, wherein the nucleic acid sequence has at least about 80% (e.g., about 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 118-122, 124-127, 129-132, 139-162, and 287-291. In some embodiments, the circular polyribonucleotide includes an open reading frame with a nucleic acid sequence encoding a SARS-CoV-2 Spike immunogen, wherein the nucleic acid sequence has at least about 90% (e.g., about 91%, 92%, 93%, 94%, 95%, 96%, 97%, 98%, 99%, or 100%) sequence identity to any one of SEQ ID NOs: 118-122, 124-127, 129-132, 139-162, and 287-291. In some embodiments, the circular polyribonucleotide includes an open reading frame with a nucleic acid sequence encoding a SARS-CoV-2 Spike immunogen, wherein the nucleic acid sequence has at least about 95% (e.g., about 96%, 97%, 98%, 99%, or 100%) sequence identity to any one of SEQ ID NOs: 118-122, 124-127, 129-132, 139-162, and 287-291. In certain embodiments, the circular polyribonucleotide includes an open reading frame with a nucleic acid sequence encoding a SARS-CoV-2 Spike immunogen, wherein the nucleic acid sequence is any one of SEQ ID NOs: 118-122, 124-127, 129-132, 139-162, and 287-291. In some embodiments, the polyribonucleotide sequence encoding the SARS-CoV-2 Spike immunogen is a fragment including a contiguous stretch of at least 300, 400, 500, 600, 700, 800, 900, 1000, 1100, 1200, 1500, 2000, 2500, 3000, 3500, 4000, or 4500 nucleotides of any one of SEQ ID NOs: 118-122, 124-127, 129-132, 139-162, and 287-291. In some embodiments, the polyribonucleotide sequence encoding the SARS-CoV-2 Spike immunogen is a fragment including a contiguous stretch of at least 50%, 60%, 70%, 80%, 90%, or 95% of the amino acids of any one of SEQ ID NOs: 118-122, 124-127, 129-132, 139-162, and 287-291.

In particular embodiments, a circular polyribonucleotide comprises a SARS-CoV-2 nonstructural protein (nsp) immunogen. In some embodiments, the circular polyribonucleotide includes an open reading frame encoding a SARS-CoV-2 nsp immunogen having an amino acid sequence that is at least about 80% (e.g., about 81%, 82%, 83%, 84%, 85%, 86%, 87%, 88%, 89%, 90%, 91%, 92%, 93%, 94%, 95%, 96%, 97%, 98%, 99%, or 100%) identical to any one of SEQ ID NOs: 291-295. In some embodiments, the circular polyribonucleotide includes an open reading frame encoding a SARS-CoV-2 nsp immunogen having an amino acid sequence that is at least about 90% (e.g., about 91%, 92%, 93%, 94%, 95%, 96%, 97%, 98%, 99%, or 100%) identical to any one of SEQ ID NOs: 291-295. In some embodiments, the circular polyribonucleotide includes an open reading frame encoding a SARS-CoV-2 nsp immunogen having an amino acid sequence that is at least about 95% (e.g., about 96%, 97%, 98%, 99%, or 100%) identical to any one of SEQ ID NOs: 291-295. In some embodiments, the circular polyribonucleotide includes an open reading frame encoding a SARS-CoV-2 immunogen having an amino acid sequence that is any one of SEQ ID NOs: 291-295.

In some embodiments, the SARS-CoV-2 nsp immunogen is an immunogenic fragment including a contiguous stretch of at least 100, 150, 200, 250, 300, 350, 400, 450, 500, 550, 600, 650, 700, 750, 800, 850, 900, 950, 1000, 1100, 1200, 1300, 1400, or 1500 amino acids of any one of SEQ ID NOs: 291-295. In some embodiments, the SARS-CoV-2 nsp immunogen is an immunogenic fragment including a contiguous stretch of at least 50%, 60%, 70%, 80%, 90%, or 95% of the amino acids of a sequence any one of SEQ ID NOs: 291-295.

In particular embodiments, the circular polyribonucleotide includes an open reading frame with a nucleic acid sequence encoding a SARS-CoV-2 nsp immunogen, wherein the nucleic acid sequence has at least about 80% (e.g., about 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 296-300. In some embodiments, the circular polyribonucleotide includes an open reading frame with a nucleic acid sequence encoding a SARS-CoV-2 nsp immunogen, wherein the nucleic acid sequence has at least about 90% (e.g., about 91%, 92%, 93%, 94%, 95%, 96%, 97%, 98%, 99%, or 100%) sequence identity to any one of SEQ ID NOs: 296-300. In some embodiments, the circular polyribonucleotide includes an open reading frame with a nucleic acid sequence encoding a SARS-CoV-2 nsp immunogen, wherein the nucleic acid sequence has at least about 95% (e.g., about 96%, 97%, 98%, 99%, or 100%) sequence identity to any one of SEQ ID NOs: 296-300. In certain embodiments, the circular polyribonucleotide includes an open reading frame with a nucleic acid sequence encoding a SARS-CoV-2 nsp immunogen, wherein the nucleic acid sequence is any one of SEQ ID NOs: 296-300, and 287-291. In some embodiments, the polyribonucleotide sequence encoding the SARS-CoV-2 nsp immunogen is a fragment including a contiguous stretch of at least 300, 400, 500, 600, 700, 800, 900, 1000, 1100, 1200, 1500, 2000, 2500, 3000, 3500, 4000, or4500 nucleotides of any one of SEQ ID NOs: 296-300. In some embodiments, the polyribonucleotide sequence encoding the SARS-CoV-2 nsp immunogen is a fragment including a contiguous stretch of at least 50%, 60%, 70%, 80%, 90%, or 95% of the amino acids of any one of SEQ ID NOs: 296-300.

The disclosure specifically contemplates that any of the DNA sequences described herein may be converted to the corresponding RNA sequence and included in an RNA molecule described herein.

TABLE 3
SARS-CoV-2 RBD Immunogen Constructs

| Amino | Nucleic | Signal | |
|---|---|---|---|
| Acid | Acid | Sequence | |
| SEQ ID | SEQ ID | SEQ ID | |
| NO: | NO: | NO: | Description |
| 63 | 112 | hEPO | RBD immunogen with hEPO signal sequence |
| | | (SEQ ID | |
| | | NO: 197) | |
| 64 | 113 | IL2 | RBD immunogen with IL2 signal sequence |
| | | (SEQ ID | |
| | | NO: 199) | |
| 65 | 114 | gLuc | RBD immunogen with gLuc signal sequence |
| | | (SEQ ID | |
| | | NO: 198) | |
| 66 | 115 | hEPO | RBD immunogen with hEPO signal sequence |
| | | (SEQ ID | |
| | | NO: 197) | |
| 67 | 116 | IL2 | RBD immunogen with IL2 signal sequence |
| | | (SEQ ID | |
| | | NO: 199) | |
| 68 | 117 | IL2 | RBD immunogen with IL2 signal sequence |
| | | (SEQ ID | |
| | | NO: 199) | |
| 74 | 123 | gLuc | RBD immunogen with gLuc signal sequence |
| | | (SEQ ID | |
| | | NO: 198) | |
| 79 | 128 | gLuc | RBD immunogen with K417N, E484K, and N501Y |
| | | (SEQ ID | mutations |
| | | NO: 198) | |
| 81 | 133 | gLuc | Two RBD immunogens, each having a gLuc signal |
| | | (SEQ ID | sequence, where the gLuc-RBD immunogens are |
| | | NO: 198) | separated by a cleavable linker including a |
| | | | furin protease cleavage site and a P2A |
| | | | self-cleaving peptide |
| 82 | 134 | gLuc | Three RBD immunogens, each having a gLuc signal |
| | | (SEQ ID | sequence, where the gLuc-RBD immunogens are each |
| | | NO: 198) | separated by a cleavable linker including a |
| | | | furin protease cleavage site and a P2A |
| | | | self-cleaving peptide |
| 83 | 135 | gLuc | Two RBD immunogens, the first having a gLuc signal |
| | | (SEQ ID | sequence, where the RBD immunogens are separated |
| | | NO: 198) | by a cleavable linker including a furin protease |
| | | | cleavage site and a P2A self-cleaving peptide |
| 84 | 136 | gLuc | Two RBD immunogens, the first having a gLuc signal |
| | | (SEQ ID | sequence, where the first two RBD immunogens are |
| | | NO: 198) | separated by a cleavable linker including a |
| | | | furin protease cleavage site and a P2A |
| | | | self-cleaving, and the second two RBD immunogens |
| | | | are separated by a P2A self-cleaving peptide |
| 85 | 137 | gLuc | Two RBD immunogens, the first having a gLuc signal |
| | | (SEQ ID | sequence, where the RBD immunogens are separated |
| | | NO: 198) | by a cleavable linker including a furin protease |
| | | | cleavage site and a P2A self-cleaving peptide, |
| | | | and including an ovalbumin peptide |
| 86 | 138 | gLuc | Two RBD immunogens, each having a gLuc signal |
| | | (SEQ ID | sequence, separated by a SCO furin cleavage |
| | | NO: 198) | site and a P2A self-cleaving peptide |
| 98 | 163 | WT | RBD immunogen having a wildtype SARS-CoV-2 |
| | | (SEQ ID | signal sequence and a T4 foldon domain |
| | | NO: 198) | |
| 99 | 164 | gLuc | RBD immunogen having a gLuc signal sequence |
| | | (SEQ ID | |
| | | NO: 198) | |
| 100 | 165 | gLuc | RBD domain having a gLuc signal sequence, a GS |
| | | (SEQ ID | linker, and a T4 foldon domain |
| | | NO: 198) | |
| 101 | 166 | gLuc | Two RBD immunogens, the first having a gLuc signal |
| | | (SEQ ID | sequence, where the two RBD immunogens are |
| | | NO: 198) | separated by a GS linker, a T4 foldon domain, and |
| | | | another GS linker |
| 102 | 167 | gLuc | RBD immunogen having a gLuc signal sequence and a |
| | | (SEQ ID | ferritin domain |
| | | NO: 198) | |
| 103 | 168 | gLuc | RBD immunogen having a gLuc signal sequence, |
| | | (SEQ ID | a GS linker, and a ferritin domain |
| | | NO: 198) | |
| 104 | 169 | gLuc | RBD immunogen having a gLuc signal sequence and |
| | | (SEQ ID | linker with a T4 foldon and ferritin domain |
| | | NO: 198) | |
| 105 | 170 | gLuc | RBD immunogen having a gLuc signal sequence and |
| | | (SEQ ID | beta-annulus peptide |
| | | NO: 198) | |
| 106 | 171 | gLuc | RBD immunogen having a gLuc signal sequence, |
| | | (SEQ ID | a GS linker, and a beta-annulus peptide |
| | | NO: 198) | |
| 107 | 172 | gLuc | RBD immunogen having a gLuc signal sequence and |
| | | (SEQ ID | beta-annulus peptide |
| | | NO: 198) | |
| 108 | 173 | gLuc | RBD immunogen having a gLuc signal sequence, |
| | | (SEQ ID | a GS linker, and a beta-annulus peptide |
| | | NO: 198) | |
| 109 | — | gLuc | RBD immunogen having a gLuc signal, an adaptor, |
| | | (SEQ ID | and a PADRE epitope |
| | | NO: 198) | |
| 110 | — | gLuc | RBD immunogen having a gLuc signal, an adaptor, |
| | | (SEQ ID | and a Tetanus helper epitope |
| | | NO: 198) | |
| 111 | 174 | gLuc | RBD immunogen having a gLuc signal sequence and a |
| | | (SEQ ID | foldon domain |
| | | NO: 198) | |

TABLE 4
SARS-CoV-2 Spike Immunogen Constructs

| Amino | Nucleic | | |
|---|---|---|---|
| Acid | Acid | | |
| SEQ ID | SEQ ID | Secretion | |
| NO: | NO: | Signal | Description |
| 69 | 118 | gLuc | Spike immunogen with gLuc signal sequence |
| | | (SEQ ID | |
| | | NO: 198) | |
| 70 | 119 | WT | Spike immunogen with wildtype SARS-CoV-2 |
| | | (SEQ ID | signal sequence |
| | | NO: 200) | |
| 71 | 120 | WT | Spike immunogen with D614G mutation and |
| | | (SEQ ID | wildtype SARS-CoV-2 signal sequence and |
| | | NO: 200) | |
| 72 | 121 | WT | Spike immunogen with wildtype SARS-CoV-2 |
| | | (SEQ ID | signal sequence and furin cleavage site |
| | | NO: 200) | |
| 73 | 122 | WT | Spike immunogen with D614G mutation, |
| | | (SEQ ID | wildtype SARS-CoV-2 signal sequence, |
| | | NO: 200) | and furin cleavage site |
| 75 | 124 | WT | Spike immunogen with wildtype SARS-CoV-2 |
| | | (SEQ ID | signal sequence |
| | | NO: 200) | |
| 76 | 125 | WT | Spike immunogen (Brazil P.1 variant) |
| | | (SEQ ID | |
| | | NO: 200) | |
| 77 | 126 | WT | Spike immunogen (South African B.1.351 |
| | | (SEQ ID | variant) |
| | | NO: 200) | |
| 78 | 127 | WT | Spike immunogen (UK B.1.1.7 variant |
| | | (SEQ ID | immunogen) |
| | | NO: 200) | |
| 80 | 129 | WT | Spike immunogen (VOC B.1.351) |
| | | (SEQ ID | |
| | | NO: 200) | |
| 87 | 130 | — | Spike immunogen |
| 88 | 131 | WT | Spike immunogen with wild-type |
| | | (SEQ ID | signal sequence |
| | | NO: 200) | |
| 89 | 132 | WT | Spike immunogen with wild-type |
| | | (SEQ ID | signal sequence |
| | | NO: 200) | |
| 90 | 139-159 | WT | Spike immunogen with wild-type signal |
| | | (SEQ ID | sequence |
| | | NO: 200) | |
| 91 | — | — | Spike chimera1-RBD:SARS-CoV-NTD:HKU3-1-S2:SARS- |
| | | | CoV-2 |
| 92 | — | — | Spike chimera 2-RBD:SARS-CoV-2-NTD:SARS-CoV, |
| | | | S2:SARS-CoV |
| 93 | — | — | Spike chimera 3-RBD:SARS-CoV-NTD:SARS-CoV2, |
| | | | S2:SARS-CoV2 |
| 94 | — | — | Spike chimera 4-RBD:RsSHC014-NTD:SARS-CoV2, |
| | | | S2:SARS-CoV2 |
| 95 | 160 | Il2 | Spike immunogen having an IL-2 signal |
| | | (SEQ ID | sequence and a T4 foldon domain |
| | | NO: 199) | |
| 96 | 161 | Il2 | Spike immunogen having an IL-2 signal |
| | | (SEQ ID | sequence and a T4 foldon domain |
| | | NO: 199) | |
| 97 | 162 | gLuc | Spike immunogen having a gLuc signal |
| | | (SEQ ID | sequence |
| | | NO: 198) | |

TABLE 5
Constructs including single open reading frame with multiple immunogens

| Amino | Nucleic | | | | | | | | | | |
|---|---|---|---|---|---|---|---|---|---|---|---|
| Acid | Acid | | | | | | | | | | |
| SEQ | SEQ | Secretion | Immunogen | Cleavage | | Secretion | Immunogen | Cleavage | Element | Secretion | Immunogen |
| ID NO: | ID NO: | Signal 1 | 1 | site 1 | Element1 | Signal 2 | 2 | site 2 | 2 | Signal 3 | 3 |
| 81 | 133 | gLuc | RBD | Furin | P2A | gLuc | RBD | N/A | N/A | N/A | N/A |
| | | (SEQ ID | | cleavage | (SEQ ID | (SEQ ID | | | | | |
| | | NO: 198) | | site | NO: 202) | NO: 198) | | | | | |
| | | | | (SEQ ID | | | | | | | |
| | | | | NO: 201) | | | | | | | |
| 82 | 134 | gLuc | RBD | Furin | P2A | gLuc | RBD | Furin | P2A | gLuc | RBD |
| | | (SEQ ID | | cleavage | (SEQ ID | (SEQ ID | | cleavage | (SEQ ID | (SEQ ID | |
| | | NO: 198) | | site | NO: 202) | NO: 198) | | site | NO: 202) | NO: 198) | |
| | | | | (SEQ ID | | | | (SEQ ID | | | |
| | | | | NO: 201) | | | | NO: 201) | | | |
| 83 | 135 | gLuc | RBD | Furin | P2A | N/A | RBD | N/A | N/A | N/A | N/A |
| | | (SEQ ID | | cleavage | (SEQ ID | | | | | | |
| | | NO: 198) | | site | NO: 202) | | | | | | |
| | | | | (SEQ ID | | | | | | | |
| | | | | NO: 201) | | | | | | | |
| 84 | 136 | gLuc | RBD | Furin | P2A | N/A | RBD | N/A | P2A | N/A | RBD |
| | | (SEQ ID | | cleavage | (SEQ ID | | | | (SEQ ID | | |
| | | NO: 198) | | site | NO: 202) | | | | NO: 202) | | |
| | | | | (SEQ ID | | | | | | | |
| | | | | NO: 201) | | | | | | | |
| 86 | 138 | gLuc | RBD | SCO Furin | P2A | gLuc | RBD | N/A | N/A | N/A | N/A |
| | | (SEQ ID | | cleavage | (SEQ ID | (SEQ ID | | | | | |
| | | NO: 198) | | site (SEQ | NO: 202) | NO: 198) | | | | | |
| | | | | ID NO: 203) | | | | | | | |
| 101 | 166 | gLuc | RBD | N/A | Foldon | N/A | RBD | N/A | N/A | N/A | N/A |
| | | (SEQ ID | | | (SEQ ID | | | | | | |
| | | NO: 198) | | | NO: 204) | | | | | | |

In some embodiments, the GC content of a nucleic acid sequence encoding a SARS-CoV-2 immunogen is at least 51% (e.g., at least 52%, 53%, 54%, 55%, 56%, 57%, 58%, 59%, or 60%). In some embodiments, the GC content of a nucleic acid sequence encoding a SARS-CoV-2 immunogen is at most 52%, 53%, 54%, 55%, 56%, 57%, 58% or 59%, or 60%. In some embodiments, the GC content of a nucleic acid sequence encoding a SARS-CoV-2 immunogen is 51% to 60%, 52% to 60%, 53% to 60%, 54% to 60%, 55% to 60%, 52% to 58%, 53% to 58%.

In some embodiments, the uridine content (for RNA) or the thymidine content (for DNA) of a nucleic acid sequence encoding a SARS-CoV-2 immunogen is more than 10% (e.g., more than 11%, 12%, 13%, 14%, 15%, 16%, 17%, 18%, 19%, 20%, 21%, 22%, 23%, 24%, or 25%). In some embodiments, the uridine content (for RNA) or the thymidine content (for DNA) of a nucleic acid sequence encoding a SARS-CoV-2 immunogen is at most 30% (e.g., at most 29%, 28%, 27%, 26%, 25%, 24%, 23%, 22%, 21%, or 20%). In some embodiments, the uridine content (for RNA) or the thymidine content (for DNA) of a nucleic acid sequence encoding a SARS-CoV-2 immunogen is 20% to 28%, 21% to 26%, 10% to 24%, 15% to 24%, 20% to 24%, 21% to 24%, 22% to 24%, 23% to 24%, 10% to 23%, 15% to 23%, 20% to 23%, 21% to 23%, or 22% to 23%.

The GC content of an expression sequence encoding the SARS-CoV-2 immunogen refers to the GC content of the expression sequence that exclusively encodes the SARS-CoV-2 immunogen with no other coding regions that encode peptides other than the SARS-CoV-2 immunogen. Likewise, the uridine content or thymidine of an expression sequence encoding the SARS-CoV-2 immunogen refers to the uridine content of the expression sequence that exclusively encodes the SARS-CoV-2 immunogen with no other coding regions that encode peptides other than the SARS-CoV-2 immunogen. In some embodiments, the calculation of the GC content or the uridine (or thymidine) content of the expression sequence encoding the SARS-CoV-2 immunogen only takes into account the continuous nucleic acid sequence that starts in a 5′ to 3′ direction from the first nucleoside of the start codon of the open reading frame that encodes the SARS-CoV-2 immunogen to the last nucleoside of the stop codon of the same open reading frame. In other embodiments, the calculation of the GC content or the uridine (or thymidine) content of the expression sequence encoding the SARS-CoV-2 immunogen only takes into account the continuous nucleic acid sequence that starts in a 5′ to 3′ direction from the first nucleoside of the codon that encodes the N-terminal end amino acid residue of the SARS-CoV-2 immunogen to the last nucleoside of the codon that encodes the C-terminal end amino acid residue of the SARS-CoV-2 immunogen.

In some embodiments, an immunogen or epitope is from a host subject (e.g., a subject for immunization) cell. For example, antibodies that block entry of a coronavirus can be produced by using an immunogen or epitope from a component of a host cell that the virus uses as an entry factor.

In some embodiments, a coronavirus epitope comprises or contains at least 4, at least 5, at least 6, at least 7, at least 8, at least 9, at least 10, at least 11, at least 12, at least 13, at least 14, at least 15, at least 16, at least 17, at least 18, at least, 19, at least 20, at least 21, at least 22, at least 23, at least 24, at least 25, at least 26, at least 27, at least 28, at least 29, or at least 30 amino acids, or more. In some embodiments, a coronavirus epitope comprises or contains at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, or at most 30 amino acids, or less. In some embodiments, a coronavirus epitope comprises or contains 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. In some embodiments, a coronavirus epitope contains 5 amino acids. In some embodiments, a coronavirus epitope contains 6 amino acids. In some embodiments, an epitope contains 7 amino acids. In some embodiments, a coronavirus epitope contains 8 amino acids. In some embodiments, an epitope can be about 8 to about 11 amino acids. In some embodiments, an epitope can be about 9 to about 22 amino acids.

The coronavirus immunogens may comprise immunogens recognized by B cells, immunogens recognized by T cells, or a combination thereof. In some embodiments, the immunogens comprise immunogens recognized by B cells. In some embodiments, the coronavirus immunogens are immunogens recognized by B cells. In some embodiments, the coronavirus immunogens comprise immunogens recognized by T cells. In some embodiments, the immunogens are immunogens recognized by T cells.

The coronavirus epitopes comprise recognized by B cells, immunogens recognized by T cells, or a combination thereof. In some embodiments, the coronavirus epitopes comprise epitopes recognized by B cells. In some embodiments, the epitopes are epitopes recognized by B cells. In some embodiments, the coronavirus epitopes comprise epitopes recognized by T cells. In some embodiments, the coronavirus epitopes are epitopes recognized by T cells.

Techniques for identifying immunogens and epitopes in silico have been disclosed, for example, in Sanchez-Trincado, et al. (2017), Fundamentals and methods for T-and B-cell epitope prediction, JOURNAL OF IMMUNOLOGY RESEARCH; Grifoni, Alba, et al., A Sequence Homology and Bioinformatic Approach Can Predict Candidate Targets for Immune Responses to SARS-CoV-2. CELL HOST & MICROBE (2020); Russi et al., and, In silico prediction of T-and B-cell epitopes in PmpD: First step towards to the design of a Chlamydia trachomatis vaccine, BIOMEDICAL JOURNAL 41.2 (2018): 109-17; Baruah, et al. Immunoinformatics-aided identification of T cell and B cell epitopes in the surface glycoprotein of 2019-nCoV. Journal of Medical Virology (2020); each of which is incorporated herein by reference in its entirety.

A circular polyribonucleotide of the disclosure may comprise sequences of any number of coronavirus immunogens and/or epitopes. A circular polyribonucleotide comprises a sequence, for example, of at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more coronavirus immunogens or epitopes (e.g., selected from any of the coronavirus immunogens and/or epitopes described herein).

In some embodiments, a circular polyribonucleotide comprises a sequence for example, of at most 1, at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, at most 500, or less coronavirus immunogens or epitopes.

In some embodiments, a circular polyribonucleotide comprises a sequence, for example, of about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 coronavirus immunogens or epitopes.

A circular polyribonucleotide may comprise a sequence for one or more coronavirus epitopes from a coronavirus immunogen. For example, a coronavirus immunogen can comprise an amino acid sequence, which can contain multiple coronavirus epitopes (e.g., epitopes recognized by B cells and/or T cells) therein, and a circular polyribonucleotide can comprise or encode one or more of those coronavirus epitopes. In some embodiments, the circular polyribonucleotide may include one or more sequences encoding a coronavirus immunogen and one or more sequences encoding immunogens that are note a coronavirus immunogen. For example, the circular polyribonucleotide may include one or more sequences encoding a coronavirus immunogen and one or more sequences encoding an immunogen from another virus (e.g., an influenza virus immunogen).

A circular polyribonucleotide comprises a sequence, for example, of at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more epitopes from one coronavirus immunogen.

In some embodiments, a circular polyribonucleotide comprises, for example, a sequence of at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, or at most 500, or less coronavirus epitopes from one coronavirus immunogen.

In some embodiments, a circular polyribonucleotide comprises a sequence, for example, of about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 coronavirus epitopes from one coronavirus immunogen.

A circular polyribonucleotide may encode variants of a coronavirus immunogen or epitope. Variants may be naturally occurring variants (for example, variants identified in sequence data from different coronavirus genera, species, isolates, or quasi species), or may be derivative sequences as disclosed herein that have been generated in silico (for example, immunogen or epitopes with one or more amino acid insertions, deletions, substitutions, or a combination thereof compared to a wild-type immunogen or epitope).

A circular polyribonucleotide comprises a sequence, for example, of at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more variants of a coronavirus immunogen or epitope.

In some embodiments, a circular polyribonucleotide comprises a sequence, for example, of at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, at most 500, or less variants of a coronavirus immunogen or epitope.

In some embodiments, a circular polyribonucleotide comprises a sequence, for example, of about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 variants of a coronavirus immunogen or epitope.

A coronavirus immunogen and/or epitope sequence of a circular polyribonucleotide can also be referred to as a coronavirus expression sequence. In some embodiments, the circular polyribonucleotide comprises one or more coronavirus expression sequences, each of which may encode a coronavirus polypeptide. The coronavirus polypeptide may be produced in substantial amounts. A coronavirus polypeptide can be a coronavirus polypeptide that is secreted from a cell, or localized to the cytoplasm, nucleus or membrane compartment of a cell. Some coronavirus polypeptides include, but are not limited to, an immunogen as disclosed herein, an epitope as disclosed herein, at least a portion of a coronavirus protein (for example, a viral envelope protein, viral matrix protein, viral spike protein, viral receptor binding domain (RBD) of a viral spike protein, viral membrane protein, viral nucleocapsid protein, viral accessory protein, a fragment thereof, or a combination thereof). In some embodiments, a coronavirus polypeptide encoded by a circular polyribonucleotide of the disclosure comprises a fragment of a coronavirus immunogen disclosed herein. In some embodiments, a coronavirus polypeptide encoded by a circular polyribonucleotide of the disclosure comprises a fusion protein comprising two or more coronavirus immunogens disclosed herein, or fragments thereof. In some embodiments, a coronavirus polypeptide encoded by a circular polyribonucleotide of the disclosure comprises a coronavirus epitope. In some embodiments, a polypeptide encoded by a circular polyribonucleotide of the disclosure comprises a fusion protein comprising two or more coronavirus epitopes disclosed herein, for example, an artificial peptide sequence comprising a plurality of predicted epitopes from one or more coronavirus s of the disclosure.

In some embodiments, exemplary coronavirus proteins that are expressed from the circular polyribonucleotide disclosed herein include a secreted protein, for example, a protein (e.g., immunogen and/or epitope) that naturally includes a signal peptide, or one that does not usually encode a signal peptide but is modified to contain one.

In some cases, the circular polyribonucleotide expresses a secretary coronavirus protein that has a short half-life in the blood, or is a protein with a subcellular localization signal, or protein with secretory signal peptide. In some cases, the circular polyribonucleotide expresses a transmembrane domain that has a short half-life in the blood, or is a protein with a subcellular localization signal, or protein with secretory peptide.

In some embodiments, the circular polyribonucleotide comprises one or more coronavirus expression sequences and is configured for persistent expression in a cell of a subject (e.g., a subject for immunization) in vivo. In some embodiments, the circular polyribonucleotide is configured such that expression of the one or more coronavirus expression sequences in the cell at a later time point is equal to or higher than an earlier time point. In such embodiments, the expression of the one or more coronavirus expression sequences is either maintained at a relatively stable level or can increase over time. In some embodiments, the expression of the coronavirus expression sequences is relatively stable for an extended period of time.

In some embodiments, the circular polyribonucleotide expresses one or more coronavirus immunogens and/or epitopes in a subject (e.g., a subject for immunization), e.g., transiently or long term. In certain embodiments, expression of the coronavirus expression sequences persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, expression of the coronavirus immunogens and/or epitopes persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 45 days, 60 days, 75 days, 90 days, or any time therebetween.

In some embodiments, the coronavirus expression sequence has a length less than 5000 bps (e.g., less than about 5000 bps, 4000 bps, 3000 bps, 2000 bps, 1000 bps, 900 bps, 800 bps, 700 bps, 600 bps, 500 bps, 400 bps, 300 bps, 200 bps, 100 bps, 50 bps, 40 bps, 30 bps, 20 bps, 10 bps, or less). In some embodiments, the coronavirus expression sequence has, independently or in addition to, a length greater than 10 bps (e.g., at least about 10 bps, 20 bps, 30 bps, 40 bps, 50 bps, 60 bps, 70 bps, 80 bps, 90 bps, 100 bps, 200 bps, 300 bps, 400 bps, 500 bps, 600 bps, 700 bps, 800 bps, 900 bps, 1000 kb, 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4 kb, 4.1 kb, 4.2 kb, 4.3 kb, 4.4 kb, 4.5 kb, 4.6 kb, 4.7 kb, 4.8 kb, 4.9 kb, 5 kb or greater).

In some embodiments, the circular polyribonucleotide encodes a plurality of immunogens (e.g., one or more, two or more, three or more, four or more, or five or more immunogens) and the plurality of immunogens share at least 90%, 91%, 92%, 93%, 94%, 95%, 96%, 97%, 98%, or 99% sequence identity. In some embodiments, the plurality of immunogens also has less than 100% sequence identity. This may be indicative of immunogens related to one another by genetic drift, as such, a single circular polyribonucleotide composition or immunogenic composition may be able to induce an immune response against a target that exists in various mutational states in a population or may induce an immune response against multiple targets having the same immunogen where the immunogen is related by genetic drift. For example, the immunogens may be related to one another by genetic drift of a target virus (e.g., a coronavirus, such as SARS-Cov-2).

### Derivatives and Fragments

An immunogen or epitope of the disclosure can comprise a wild-type sequence. When describing an immunogen or epitope, the term “wild type” refers to a sequence (e.g., an amino acid sequence) that is naturally occurring and encoded by a genome (e.g., a coronavirus genome). A coronavirus can have one wild-type sequence, or two or more wild type sequences (for example, with one canonical wild-type sequence present in a reference coronavirus genome, and additional variant wild-type sequences present that have arisen from mutations).

When describing an immunogen or epitope, the terms “derivative” and “derived from” refer to a sequence (e.g., amino acid sequence) that differs from a wild-type sequence by one or more amino acids, for example, containing one or more amino acid insertions, deletions, and/or substitutions relative to a wildtype sequence.

An immunogen or epitope derivative sequence is a sequence that has at least 60%, 70%, 75%, 80%, 85%, 90%, 91%, 92%, 93%, 94%, 95%, 96%, 97%, 98%, 99%, or more sequence identity to a wild-type sequence, for example, a wild type protein, immunogen, or epitope sequence.

In some embodiments, an immunogen or epitope contains one or more amino acid insertions, deletions, substitutions, or a combination thereof that affect the structure of an encoded protein. In some embodiments, an immunogen or epitope contains one or more amino acid insertions, deletions, substitutions, or a combination thereof that affect the function of an encoded protein. In some embodiments, an immunogen or epitope contains one or more amino acid insertions, deletions, substitutions, or a combination thereof that affect the expression or processing of an encoded protein by a cell.

Amino acid insertions, deletions, substitutions, or a combination thereof can introduce a site for a post-translational modification (for example, introduce a glycosylation, ubiquitination, phosphorylation, nitrosylation, methylation, acetylation, amidation, hydroxylation, sulfation, or lipidation site, or a sequence that is targeted for cleavage). In some embodiments, amino acid insertions, deletions, substitutions, or a combination thereof remove a site for a post-translational modification (for example, remove a glycosylation, ubiquitination, phosphorylation, nitrosylation, methylation, acetylation, amidation, hydroxylation, sulfation, or lipidation site, or a sequence that is targeted for cleavage). In some embodiments, amino acid insertions, deletions, substitutions, or a combination thereof modify a site for a post-translational modification (for example, modify a site to alter the efficiency or characteristics of glycosylation, ubiquitination, phosphorylation, nitrosylation, methylation, acetylation, amidation, hydroxylation, sulfation, or lipidation site, or cleavage).

An amino acid substitution can be a conservative or a non-conservative substitution. A conservative amino acid substitution can be a substitution of one amino acid for another amino acid of similar biochemical properties (e.g., charge, size, and/or hydrophobicity). A non-conservative amino acid substitution can be a substitution of one amino acid for another amino acid with different biochemical properties (e.g., charge, size, and/or hydrophobicity). A conservative amino acid change can be, for example, a substitution that has minimal effect on the secondary or tertiary structure of a polypeptide. A conservative amino acid change can be an amino acid change from one hydrophilic amino acid to another hydrophilic amino acid. Hydrophilic amino acids can include Thr (T), Ser (S), His (H), Glu (E), Asn (N), Gln (Q), Asp (D), Lys (K) and Arg (R). A conservative amino acid change can be an amino acid change from one hydrophobic amino acid to another hydrophilic amino acid. Hydrophobic amino acids can include Ile (I), Phe (F), Val (V), Leu (L), Trp (W), Met (M), Ala (A), Gly (G), Tyr (Y), and Pro (P). A conservative amino acid change can be an amino acid change from one acidic amino acid to another acidic amino acid. Acidic amino acids can include Glu (E) and Asp (D). A conservative amino acid change can be an amino acid change from one basic amino acid to another basic amino acid. Basic amino acids can include His (H), Arg (R) and Lys (K). A conservative amino acid change can be an amino acid change from one polar amino acid to another polar amino acid. Polar amino acids can include Asn (N), Gln (Q), Ser (S) and Thr (T). A conservative amino acid change can be an amino acid change from one nonpolar amino acid to another nonpolar amino acid. Nonpolar amino acids can include Leu (L), Val (V), Ile (I), Met (M), Gly (G) and Ala (A). A conservative amino acid change can be an amino acid change from one aromatic amino acid to another aromatic amino acid. Aromatic amino acids can include Phe (F), Tyr (Y) and Trp (W). A conservative amino acid change can be an amino acid change from one aliphatic amino acid to another aliphatic amino acid. Aliphatic amino acids can include Ala (A), Val (V), Leu (L) and Ile (I). In some embodiments, a conservative amino acid substitution is an amino acid change from one amino acid to another amino acid within one of the following groups: Group I: ala, pro, Gly, Gln, Asn, Ser, Thr; Group II: Cys, Ser, Tyr, Thr; Group III: Val, lie, Leu, Met, Ala, Phe; Group IV: Lys, Arg, His; Group V: Phe, Tyr, Trp, His; and Group VI: Asp, Glu.

In some embodiments, an immunogen derivative or epitope derivative of the disclosure comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, or at least 100 amino acid deletions relative to a sequence disclosed herein (e.g., a wild type sequence).

In some embodiments, an immunogen derivative or epitope derivative of the disclosure comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid substitutions relative to a sequence disclosed herein (e.g., a wild-type sequence).

In some embodiments, an immunogen derivative or epitope derivative of the disclosure comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to a sequence disclosed herein (e.g., a wild-type sequence).

In some embodiments, an immunogen derivative or epitope derivative of the disclosure comprises 1-2, 1-3, 1-4, 1-5, 1-6, 1-7, 1-8, 1-9, 1-10, 1-15, 1-20, 1-30, 1-40, 2-3, 2-4, 2-5, 2-6, 2-7, 2-8, 2-9, 2-10, 2-15, 2-20, 2-30, 2-40, 3-3, 3-4, 3-5, 3-6, 3-7, 3-8, 3-9, 3-10, 3-15, 3-20, 3-30, 3-40, 5-6, 5-7, 5-8, 5-9, 5-10, 5-15, 5-20, 5-30, 5-40, 10-15, 15-20, or 20-25 amino acid substitutions relative to a sequence disclosed herein (e.g., a wild-type sequence).

In some embodiments, an immunogen derivative or epitope derivative of the disclosure comprises 1, 2, 3, 4, 5, 6, 7, 8, 9, 10, 11, 12, 13, 14, 15, 16, 17, 18, 19, or 20 amino acid substitutions relative to a sequence disclosed herein (e.g., a wild-type sequence).

The one or more amino acid substitutions can be at the N-terminus, the C-terminus, within the amino acid sequence, or a combination thereof. The amino acid substitutions can be contiguous, non-contiguous, or a combination thereof.

In some embodiments, an immunogen derivative or epitope derivative of the disclosure comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, at most 50, at most 60, at most 70, at most 80, at most 90, at most 100, at most 120, at most 140, at most 160, at most 180, or at most 200 amino acid deletions relative to a sequence disclosed herein (e.g., a wild type sequence).

In some embodiments, an immunogen derivative or epitope derivative of the disclosure comprises 1-2, 1-3, 1-4, 1-5, 1-6, 1-7, 1-8, 1-9, 1-10, 1-15, 1-20, 1-30, 1-40, 2-3, 2-4, 2-5, 2-6, 2-7, 2-8, 2-9, 2-10, 2-15, 2-20, 2-30, 2-40, 3-3, 3-4, 3-5, 3-6, 3-7, 3-8, 3-9, 3-10, 3-15, 3-20, 3-30, 3-40, 5-6, 5-7, 5-8, 5-9, 5-10, 5-15, 5-20, 5-30, 5-40, 10-15, 15-20, 20-25, 20-30, 30-50, 50-100, or 100-200 amino acid deletions relative to a sequence disclosed herein (e.g., a wild type sequence).

In some embodiments, an immunogen derivative or epitope derivative of the disclosure comprises 1, 2, 3, 4, 5, 6, 7, 8, 9, 10, 11, 12, 13, 14, 15, 16, 17, 18, 19, or 20 amino acid deletions relative to a sequence disclosed herein (e.g., a wild-type sequence).

The one or more amino acid deletions can be at the N-terminus, the C-terminus, within the amino acid sequence, or a combination thereof. The amino acid deletions can be contiguous, non-contiguous, or a combination thereof.

In some embodiments, an immunogen derivative or epitope derivative of the disclosure comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid insertions relative to a sequence disclosed herein (e.g., a wild type sequence).

In some embodiments, an immunogen derivative or epitope derivative of the disclosure comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to a sequence disclosed herein (e.g., a wild type sequence).

In some embodiments, an immunogen derivative or epitope derivative of the disclosure comprises 1-2, 1-3, 1-4, 1-5, 1-6, 1-7, 1-8, 1-9, 1-10, 1-15, 1-20, 1-30, 1-40, 2-3, 2-4, 2-5, 2-6, 2-7, 2-8, 2-9, 2-10, 2-15, 2-20, 2-30, 2-40, 3-3, 3-4, 3-5, 3-6, 3-7, 3-8, 3-9, 3-10, 3-15, 3-20, 3-30, 3-40, 5-6, 5-7, 5-8, 5-9, 5-10, 5-15, 5-20, 5-30, 5-40, 10-15, 15-20, or 20-25 amino acid insertions relative to a sequence disclosed herein (e.g., a wild type sequence).

In some embodiments, an immunogen derivative or epitope derivative of the disclosure comprises 1, 2, 3, 4, 5, 6, 7, 8, 9, 10, 11, 12, 13, 14, 15, 16, 17, 18, 19, or 20 amino acid insertions relative to a sequence disclosed herein (e.g., a wild-type sequence).

The one or more amino acid insertions can be at the N-terminus, the C-terminus, within the amino acid sequence, or a combination thereof. The amino acid insertions can be contiguous, non-contiguous, or a combination thereof.

### Circular Polyribonucleotide Elements

The circular polyribonucleotide comprises the elements as described below as well as the coronavirus immunogen or epitope as described herein. In some embodiments, the circular polyribonucleotide includes any feature, or any combination of features as disclosed in International Patent Publication No. WO2019/118919, which is hereby incorporated by reference in its entirety.

In some embodiments, the circular polyribonucleotide is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides.

In some embodiments, the circular polyribonucleotide is between 500 nucleotides and 20,000 nucleotides, between 1,000 and 20,000 nucleotides, between 2,000 and 20,000 nucleotides, or between 5,000 and 20,000 nucleotides. In some embodiments, the circular polyribonucleotide is between 500 nucleotides and 10,000 nucleotides, between 1,000 and 10,000 nucleotides, between 2,000 and 10,000 nucleotides, or between 5,000 and 10,000 nucleotides.

### Internal Ribosome Entry Sites

In some embodiments, a circular or linear polyribonucleotide described herein includes one or more internal ribosome entry site (IRES) elements. In some embodiments, the IRES is operably linked to one or more expression sequences (e.g., each IRES is operably linked to one or more expression sequences, where each expression sequence optionally encodes an immunogen, such as a coronavirus immunogen). In embodiments, the IRES is located between a heterologous promoter and the 5′ end of a coding sequence (e.g., a coding sequence encoding a coronavirus immunogen).

A suitable IRES element to include in a polyribonucleotide includes an RNA sequence capable of engaging a eukaryotic ribosome. In some embodiments, the IRES element is at least about 5 nt, at least about 8 nt, at least about 9 nt, at least about 10 nt, at least about 15 nt, at least about 20 nt, at least about 25 nt, at least about 30 nt, at least about 40 nt, at least about 50 nt, at least about 100 nt, at least about 200 nt, at least about 250 nt, at least about 350 nt, or at least about 500 nt.

In some embodiments, the IRES element is derived from the DNA of an organism including, but not limited to, a virus, a mammal, and a Drosophila. Such viral DNA may be derived from, but is not limited to, picomavirus complementary DNA (cDNA), with encephalomyocarditis virus (EMCV) cDNA and poliovirus cDNA. In one embodiment, Drosophila DNA from which an IRES element is derived includes, but is not limited to, an Antennapedia gene from Drosophila melanogaster.

In some embodiments, the IRES sequence is an IRES sequence of Taura syndrome virus, Triatoma virus, Theiler's encephalomyelitis virus, simian Virus 40, Solenopsis invicta virus 1, Rhopalosiphum padi virus, Reticuloendotheliosis virus, fuman poliovirus 1, Plautia stall intestine virus, Kashmir bee virus, Human rhinovirus 2 (HRV-2), Homalodisca coagulata virus-1, Human Immunodeficiency Virus type 1, Homalodisca coagulata virus-1, Himetobi P virus, Hepatitis C virus, Hepatitis A virus, Hepatitis GB virus, foot and mouth disease virus, Human enterovirus 71, Equine rhinitis virus, Ectropis obliqua picorna-like virus, Encephalomyocarditis virus (EMCV), Drosophila C Virus, Crucifer tobamo virus, Cricket paralysis virus, Bovine viral diarrhea virus 1, Black Queen Cell Virus, Aphid lethal paralysis virus, Avian encephalomyelitis virus (AEV), Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1 R, Human BAG-I, Human BCL2, Human BiP, Human c-IAPI, Human c-myc, Human eIF4G, Mouse NDST4 L, Human LEF1, Mouse HIF1 alpha, Human n.myc, Mouse Gtx, Human p27kipl, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Salivirus, Cosavirus, Parechovirus, Drosophila hairless, S. cerevisiae TFIID, S. cerevisiae YAP1, Human c-src, Human FGF-1, Simian picomavirus, Turnip crinkle virus, Aichivirus, Crohivirus, Echovirus 11, an aptamer to eIF4G, Coxsackievirus B3 (CVB3) or Coxsackievirus A (CVB1/2). In yet another embodiment, the IRES is an IRES sequence of Coxsackievirus B3 (CVB3). In a further embodiment, the IRES is an IRES sequence of Encephalomyocarditis virus. In a further embodiment, the IRES is an IRES sequence of Theiler's encephalomyelitis virus.

The IRES sequence may have a modified sequence in comparison to the wild-type IRES sequence. In some embodiments, when the last nucleotide of the wild-type IRES is not a cytosine nucleic acid residue, the last nucleotide of the wild-type IRES sequence may be modified such that it is a cytosine residue. For example, the IRES sequence may be a CVB3 IRES sequence wherein the terminal adenosine residue is modified to cytosine residue. In some embodiments, the modified CVB3 IRES may have the nucleic acid sequence of:

(SEQ ID NO: 305)
TTAAAACAGCCTGTGGGTTGATCCCACCCACAGGCCCATTGGGCGCTAG
CACTCTGGTATCACGGTACCTTTGTGCGCCTGTTTTATACCCCCTCCCC
CAACTGTAACTTAGAAGTAACACACACCGATCAACAGTCAGCGTGGCAC
ACCAGCCACGTTTTGATCAAGCACTTCTGTTACCCCGGACTGAGTATCA
ATAGACTGCTCACGCGGTTGAAGGAGAAAGCGTTCGTTATCCGGCCAAC
TACTTCGAAAAACCTAGTAACACCGTGGAAGTTGCAGAGTGTTTCGCTC
AGCACTACCCCAGTGTAGATCAGGTCGATGAGTCACCGCATTCCCCACG
GGCGACCGTGGCGGTGGCTGCGTTGGCGGCCTGCCCATGGGGAAACCCA
TGGGACGCTCTAATACAGACATGGTGCGAAGAGTCTATTGAGCTAGTTG
GTAGTCCTCCGGCCCCTGAATGCGGCTAATCCTAACTGCGGAGCACACA
CCCTCAAGCCAGAGGGCAGTGTGTCGTAACGGGCAACTCTGCAGCGGAA
CCGACTACTTTGGGTGTCCGTGTTTCATTTTATTCCTATACTGGCTGCT
TATGGTGACAATTGAGAGATCGTTACCATATAGCTATTGGATTGGCCAT
CCGGTGACTAATAGAGCTATTATATATCCCTTTGTTGGGTTTATACCAC
TTAGCTTGAAAGAGGTTAAAACATTACAATTCATTGTTAAGTTGAATAC
AGCAAC

In some embodiments, the IRES sequence is an Enterovirus 71 (EV17) IRES. In some embodiments, the terminal guanosine residue of the EV17 IRES sequence is modified to a cytosine residue. In some embodiments, the modified EV71 IRES may have the nucleic acid sequence of:

(SEQ ID NO: 306)
TTAAAACAGCTGTGGGTTGTCACCCACCCACAGGGTCCACTGGGCGCTA
GTACACTGGTATCTCGGTACCTTTGTACGCCTGTTTTATACCCCCTCCC
TGATTTGCAACTTAGAAGCAACGCAAACCAGATCAATAGTAGGTGTGAC
ATACCAGTCGCATCTTGATCAAGCACTTCTGTATCCCCGGACCGAGTAT
CAATAGACTGTGCACACGGTTGAAGGAGAAAACGTCCGTTACCCGGCTA
ACTACTTCGAGAAGCCTAGTAACGCCATTGAAGTTGCAGAGTGTTTCGC
TCAGCACTCCCCCCGTGTAGATCAGGTCGATGAGTCACCGCATTCCCCA
CGGGCGACCGTGGCGGTGGCTGCGTTGGCGGCCTGCCTATGGGGTAACC
CATAGGACGCTCTAATACGGACATGGCGTGAAGAGTCTATTGAGCTAGT
TAGTAGTCCTCCGGCCCCTGAATGCGGCTAATCCTAACTGCGGAGCACA
TACCCTTAATCCAAAGGGCAGTGTGTCGTAACGGGCAACTCTGCAGCGG
AACCGACTACTTTGGGTGTCCGTGTTTCTTTTTATTCTTGTATTGGCTG
CTTATGGTGACAATTAAAGAATTGTTACCATATAGCTATTGGATTGGCC
ATCCAGTGTCAAACAGAGCTATTGTATATCTCTTTGTTGGATTCACACC
TCTCACTCTTGAAACGTTACACACCCTCAATTACATTATACTGCTGAAC
ACGAAGCGGCCACC

In some embodiments, the polyribonucleotide includes at least one IRES flanking at least one (e.g., 2, 3, 4, 5 or more) expression sequence. In some embodiments, the IRES flanks both sides of at least one (e.g., 2, 3, 4, 5 or more) expression sequence. In some embodiments, the polyribonucleotide includes one or more IRES sequences on one or both sides of each expression sequence, leading to separation of the resulting peptide(s) and or polypeptide(s). For example, a polyribonucleotide described herein may include a first IRES operably linked to a first expression sequence (e.g., encoding a first immunogen, such as a first coronavirus immunogen) and a second IRES operably linked to a second expression sequence (e.g., encoding a second immunogen, such as a second coronavirus immunogen).

In some embodiments, a polyribonucleotide described herein includes an IRES (e.g., an IRES operably linked to a coding region). For example, the polyribonucleotide may include any IRES as described in Chen et al. Mol. Cell 81(20):4300-4318, 2021; Jopling et al. Oncogene 20:2664-2670, 2001; Baranick et al. PNAS 105(12):4733-4738, 2008; Lang et al. Molecular Biology of the Cell 13(5):1792-1801, 2002; Dorokhov et al. PNAS 99(8):5301-5306, 2002; Wang et al. Nucleic Acids Research 33(7):2248-2258, 2005; Petz et al. Nucleic Acids Research 35(8):2473-2482, 2007, Chen et al. SCIENCE 268:415-417, 1995; Fan et al. NATURE COMMUNICATION 13(1):3751-3765, 2022, and International Publication No. WO2021/263124 each of which is hereby incorporated by reference in their entirety.

### Signal Sequences

In some embodiments, immunogens expressed from a circular or linear polyribonucleotide disclosed herein include a secreted protein, for example, a protein that naturally includes a signal sequence, or one that does not usually encode a signal sequence but is modified to contain one. In some embodiments, the immunogen(s) includes a secretion signal. For example, the secretion signal may be the naturally encoded secretion signal for a secreted protein. In another example, the secretion signal may be a modified secretion signal for a secreted protein. In other embodiments, the immunogen(s) do not include a secretion signal.

In some embodiments, a polyribonucleotide encodes multiple copies of the same immunogen (e.g., one, two, three, four, five, six, seven, eight, nine, ten, or more). In some embodiments, at least one copy of the immunogen includes a signal sequence and at least one copy of the immunogen does not include a signal sequence. In some embodiments, a circular polyribonucleotide encodes plurality of immunogens, where at least one of the plurality of immunogens includes a signal sequence and at least one copy of the plurality of immunogens does not include a signal sequence.

In some embodiments, the signal sequence is a wild-type signal sequence that is present on the N-terminus of the corresponding wild-type immunogen, e.g., when expressed endogenously. In some embodiments, the signal sequence is heterologous to the immunogen, e.g., is not present when the wild-type immunogen is expressed endogenously. A polyribonucleotide sequence encoding an immunogen may be modified to remove the nucleotide sequence encoding a wild-type signal sequence and/or add a sequence encoding a heterologous signal sequence.

The circular polyribonucleotide may further include one or more adjuvants, each with or without a signal sequence. In some embodiments, the circular polyribonucleotide encodes at least one adjuvant and at least one immunogen. In some embodiments, the at least one encoded adjuvant includes a signal sequence and the at least one encoded immunogen does not include a signal sequence. In some embodiments, the at least one encoded adjuvant includes a signal sequence and the at least one encoded immunogen includes a signal sequence. In some embodiments, the at least one encoded adjuvant does not include a signal sequence and the at least one encoded immunogen includes a signal sequence. In some embodiments, neither the encoded adjuvant nor the encoded immunogen includes a signal sequence.

In some embodiments, the signal sequence is a wild-type signal sequence that is present on the N-terminus of the corresponding wild-type adjuvant, e.g., when expressed endogenously. In some embodiments, the signal sequence is heterologous to the adjuvant, e.g., is not present when the wild-type adjuvant is expressed endogenously. A polyribonucleotide sequence encoding an adjuvant may be modified to remove the nucleotide sequence encoding a wild-type signal sequence and/or add a sequence encoding a heterologous signal sequence.

A polypeptide encoded by a polyribonucleotide (e.g., immunogen or an adjuvant encoded by a polyribonucleotide) may include a signal sequence that directs the immunogen or adjuvant to the secretory pathway. In some embodiments, the signal sequence may direct the immunogen or adjuvant to reside in certain organelles (e.g., the endoplasmic reticulum, Golgi apparatus, or endosomes). In some embodiments, the signal sequence directs the immunogen or adjuvant to be secreted from the cell. For secreted proteins, the signal sequence may be cleaved after secretion, resulting in a mature protein. In other embodiments, the signal sequence may become embedded in the membrane of the cell or certain organelles, creating a transmembrane segment that anchors the protein to the membrane of the cell, endoplasmic reticulum, or Golgi apparatus. In certain embodiments, the signal sequence of a transmembrane protein is a short sequence at the N-terminal of the polypeptide. In other embodiments, the first transmembrane domain acts as the first signal sequence, which targets the protein to the membrane.

In some embodiments, the secretion signal is human interleukin-2 (IL-2) secretion signal. In some embodiments, the IL-2 secretion signal has an amino acid sequence of at least 90% sequence identity to MYRMQLLSCIALSLALVTNS (SEQ ID NO: 199). In some embodiments, the IL2 secretion signal has an amino acid sequence of at least 95% sequence identity to SEQ ID NO: 199. In some embodiments, the IL-2 secretion signal has an amino acid sequence of at least 99% sequence identity to SEQ ID NO: 199. In some embodiments, the IL-2 secretion signal has an amino acid sequence of 100% sequence identity to SEQ ID NO: 199.

In some embodiments, the secretion signal is Gaussia luciferase secretion signal. In some embodiments, the Gaussia luciferase secretion signal has an amino acid sequence of at least 90% sequence identity of MGVKVLFALICIAVAEAK (SEQ ID NO: 198). In some embodiments, the Gaussia luciferase secretion signal has an amino acid sequence of at least 95% sequence identity of SEQ ID NO: 198. In some embodiments, the Gaussia luciferase secretion signal has an amino acid sequence of at least 99% sequence identity of SEQ ID NO: 198. In some embodiments, the Gaussia luciferase secretion signal has an amino acid sequence of 100% sequence identity of SEQ ID NO: 198.

In some embodiments, the secretion signal is an EPO (e.g., a human EPO) secretion signal. In some embodiments, the EPO secretion signal has an amino acid sequence of at least 90% sequence identity of MGVHECPAWLWLLLSLLSLPLGLPVLGA (SEQ ID NO: 197). In some embodiments, the EPO secretion signal has an amino acid sequence of at least 95% sequence identity of SEQ ID NO: 197. In some embodiments, the EPO secretion signal has an amino acid sequence of at least 99% sequence identity of SEQ ID NO: 197. In some embodiments, the EPO secretion signal has an amino acid sequence of 100% sequence identity of SEQ ID NO: 197.

In some embodiments, the secretion signal is a wildtype SARS-CoV-2 secretion signal. In some embodiments, the wildtype SARS-CoV-2 secretion signal has an amino acid sequence of at least 90% sequence identity of MFVFLVLLPLVSS (SEQ ID NO: 200). In some embodiments, the wildtype SARS-CoV-2 secretion signal has an amino acid sequence of at least 95% sequence identity of SEQ ID NO: 200. In some embodiments, the wildtype SARS-CoV-2 secretion signal has an amino acid sequence of at least 99% sequence identity of SEQ ID NO: 200. In some embodiments, the wildtype SARS-CoV-2 secretion signal has an amino acid sequence of 100% sequence identity of SEQ ID NO: 200.

In some embodiments, an adjuvant encoded by a polyribonucleotide includes a secretion signal sequence. In some embodiments, an immunogen encoded by a polyribonucleotide includes either a secretion signal sequence, a transmembrane insertion signal sequence, or does not include a signal sequence.

### Regulatory Elements

A regulatory element may include a sequence that is located adjacent to an expression sequence that encodes an expression product. A regulatory element may be operably linked to the adjacent sequence. A regulatory element may increase an amount of product expressed as compared to an amount of the expressed product when no regulatory element is present. A regulatory element may be used to increase the expression of one or more immunogen(s) and/or adjuvant(s) encoded by a polyribonucleotide. Likewise, a regulatory element may be used to decrease the expression of one or more immunogen(s) and/or adjuvant(s) encoded by a polyribonucleotide. In some embodiments, a regulatory element may be used to increase expression of an immunogen and/or adjuvant and another regulatory element may be used to decrease expression of another immunogen and/or adjuvant on the same polyribonucleotide. In addition, one regulatory element can increase an amount of product (e.g., an immunogen or adjuvants) expressed for multiple expression sequences attached in tandem. Hence, one regulatory element can enhance the expression of one or more expression sequences (e.g., immunogens or adjuvants). Multiple regulatory elements can also be used, for example, to differentially regulate expression of different expression sequences.

In some embodiments, a regulatory element as provided herein can include a selective translation sequence. As used herein, the term “selective translation sequence” refers to a nucleic acid sequence that selectively initiates or activates translation of an expression sequence in the polyribonucleotide, for instance, certain riboswitch aptazymes. A regulatory element can also include a selective degradation sequence. As used herein, the term “selective degradation sequence” refers to a nucleic acid sequence that initiates degradation of the polyribonucleotide, or an expression product of the polyribonucleotide. In some embodiments, the regulatory element is a translation modulator. A translation modulator can modulate translation of the expression sequence in the polyribonucleotide. A translation modulator can be a translation enhancer or suppressor. In some embodiments, a translation initiation sequence can function as a regulatory element.

In some embodiments, a polyribonucleotide produces stoichiometric ratios of expression products. Rolling circle translation continuously produces expression products at substantially equivalent ratios. In some embodiments, the polyribonucleotide has a stoichiometric translation efficiency, such that expression products are produced at substantially equivalent ratios. In some embodiments, the polyribonucleotide has a stoichiometric translation efficiency of multiple expression products, e.g., products from 2, 3, 4, 5, 6, 7, 8, 9, 10, 11, 12, or more expression sequences. In some embodiments, the polyribonucleotide produces substantially different ratios of expression products. For example, the translation efficiency of multiple expression products may have a ratio of 1:10,000; 1:7000, 1:5000, 1:1000, 1:700, 1:500, 1:100, 1:50, 1:10, 1:5, 1:4, 1:3 or 1:2. In some embodiments, the ratio of multiple expression products may be modified using a regulatory element.

Further examples of regulatory elements are described in paragraphs [0154]-[0161] of International Patent Publication No. WO2019/118919, which is hereby incorporated by reference in its entirety.

### Cleavage Domains

A circular or linear polyribonucleotide of the disclosure can include a cleavage domain (e.g., a stagger element or a cleavage sequence).

As used herein, the term “stagger element” is a moiety, such as a nucleotide sequence, that induces ribosomal pausing during translation. In some embodiments, the stagger element is a non-conserved sequence of amino-acids with a strong alpha-helical propensity followed by the consensus sequence -D(V/I)ExNPG P (SEQ ID NO: 52), where x=any amino acid. In some embodiments, the stagger element may include a chemical moiety, such as glycerol, a non-nucleic acid linking moiety, a chemical modification, a modified nucleic acid, or any combination thereof. In some embodiments, a circular or linear polyribonucleotide includes at least one stagger element adjacent to an expression sequence, such as a sequence encoding a coronavirus immunogen. In some embodiments, the circular or linear polyribonucleotide includes a stagger element adjacent to each expression sequence. In some embodiments, the stagger element is present on one or both sides of each expression sequence, leading to separation of the expression products, e.g., immunogen(s) and/or adjuvant(s). In some embodiments, the stagger element is a portion of the one or more expression sequences. In some embodiments, the circular or linear polyribonucleotide includes one or more expression sequences (e.g., immunogen(s) and/or adjuvant(s)), and each of the one or more expression sequences is separated from a succeeding expression sequence (e.g., immunogen(s) and/or adjuvant(s) by a stagger element on the circular or linear polyribonucleotide. In some embodiments, the stagger element prevents generation of a single polypeptide (a) from two rounds of translation of a single expression sequence or (b) from one or more rounds of translation of two or more expression sequences. In some embodiments, the stagger element is a sequence separate from the one or more expression sequences. In some embodiments, the stagger element includes a portion of an expression sequence of the one or more expression sequences.

Examples of stagger elements are described in paragraphs [0172]-[0175] of International Patent Publication No. WO2019/118919, which is hereby incorporated by reference in its entirety.

In some embodiments, the plurality of immunogens and/or adjuvants encoded by a circular ribonucleotide may be separated by an IRES between each immunogen (e.g., each immunogen is operably linked to a separate IRES). For example, a circular polyribonucleotide may include a first IRES operable linked to a first expression sequence and a second IRES operably linked to a second expression sequence. The IRES may be the same IRES between all immunogens. The IRES may be different between different immunogens.

In some embodiments, the plurality of immunogens and/or adjuvants may be separated by a 2A self-cleaving peptide. For example, a circular polyribonucleotide may encode an IRES operably linked to an open reading frame encoding a first immunogen, a 2A, and a second immunogen. In some embodiments, the 2A may have a sequence of GSGATNFSLLKQAGDVEENPGP (SEQ ID NO: 202).

In some embodiments, the plurality of immunogens and/or adjuvants may be separated by a protease cleavage site (e.g., a furin cleavage site). For example, a circular polyribonucleotide may encode an IRES operably linked to an open reading frame encoding a first immunogen, a protease cleavage site (e.g., a furin cleavage site), and a second immunogen. In some embodiments, the furin cleavage site may have a sequence of GRLRR (SEQ ID NO: 201). In some embodiments, the furin cleavage site may have a sequence of GRLRR (SEQ ID NO: 203).

In some embodiments, the plurality of immunogens and/or adjuvants may be separated by a 2A self-cleaving peptide and a protease cleavage site (e.g., a furin cleavage site). For example, a circular polyribonucleotide may encode an IRES operably linked to an open reading frame encoding a first immunogen, a 2A, a protease cleavage site (e.g., a furin cleavage site), and a second immunogen. A circular polyribonucleotide may also encode an IRES operably linked to an open reading frame encoding a first immunogen, a protease cleavage site (e.g., a furin cleavage site), a 2A, and a second immunogen. A tandem 2A and furin cleavage site may be referred to as a furin-2A (which includes furin-2A or 2A-furin, arranged in either orientation).

Furthermore, the plurality of immunogens and/or adjuvants encoded by the circular ribonucleotide may be separated by both IRES and 2A sequences. For example, an IRES may be between one immunogen and/or adjuvant and a second immunogen and/or adjuvant while a 2A peptide may be between the second immunogen and/or adjuvant and the third immunogen and/or adjuvant. The selection of a particular IRES or 2A self-cleaving peptide may be used to control the expression level of immunogen and/or adjuvant under control of the IRES or 2A sequence. For example, depending on the IRES and or 2A peptide selected, expression on the polypeptide may be higher or lower. To avoid production of a continuous expression product, e.g., immunogen and/or adjuvant, while maintaining rolling circle translation, a stagger element may be included to induce ribosomal pausing during translation. In some embodiments, the stagger element is at 3′ end of at least one of the one or more expression sequences. The stagger element can be configured to stall a ribosome during rolling circle translation of the circular or linear polyribonucleotide. The stagger element may include, but is not limited to a 2A-like, or CHYSEL (SEQ ID NO: 175) (cis-acting hydrolase element) sequence. In some embodiments, the stagger element encodes a sequence with a C-terminal consensus sequence that is X₁X₂X₃EX₅NPGP, where X₁ is absent or G or H, X₂ is absent or D or G, X₃ is D or V or I or S or M, and X₅ is any amino acid (SEQ ID NO: 176). Some non-limiting examples of stagger elements includes GDVESNPGP (SEQ ID NO: 177), GDIEENPGP (SEQ ID NO: 178), VEPNPGP (SEQ ID NO: 179), IETNPGP (SEQ ID NO: 180), GDIESNPGP (SEQ ID NO: 181), GDVELNPGP (SEQ ID NO: 182), GDIETNPGP (SEQ ID NO: 183), GDVENPGP (SEQ ID NO: 184), GDVEENPGP (SEQ ID NO: 185), GDVEQNPGP (SEQ ID NO: 186), IESNPGP (SEQ ID NO: 187), GDIELNPGP (SEQ ID NO: 188), HDIETNPGP (SEQ ID NO: 189), HDVETNPGP (SEQ ID NO: 190), HDVEMNPGP (SEQ ID NO: 191), GDMESNPGP (SEQ ID NO: 192), GDVETNPGP (SEQ ID NO: 193), GDIEQNPGP (SEQ ID NO: 194), and DSEFNPGP (SEQ ID NO: 195).

In some embodiments, a stagger element described herein cleaves an expression product, such as between G and P of the consensus sequence described herein. As one non-limiting example, the circular or linear polyribonucleotide includes at least one stagger element to cleave the expression product. In some embodiments, the circular or linear polyribonucleotide includes a stagger element adjacent to at least one expression sequence. In some embodiments, the circular or linear polyribonucleotide includes a stagger element after each expression sequence. In some embodiments, the circular or linear polyribonucleotide includes a stagger element is present on one or both sides of each expression sequence, leading to translation of individual peptide(s) and or polypeptide(s) from each expression sequence.

In some embodiments, a stagger element includes one or more modified nucleotides or unnatural nucleotides that induce ribosomal pausing during translation. Unnatural nucleotides may include peptide nucleic acid (PNA), Morpholino and locked nucleic acid (LNA), as well as glycol nucleic acid (GNA) and threose nucleic acid (TNA). Examples such as these are distinguished from naturally occurring DNA or RNA by changes to the backbone of the molecule. Exemplary modifications can include any modification to the sugar, the nucleobase, the internucleoside linkage (e.g., to a linking phosphate/to a phosphodiester linkage/to the phosphodiester backbone), and any combination thereof that can induce ribosomal pausing during translation. Some of the exemplary modifications provided herein are described elsewhere herein.

In some embodiments, a stagger element is present in a circular or linear polyribonucleotide in other forms. For example, in some exemplary circular or linear polyribonucleotides, a stagger element includes a termination element of a first expression sequence in the circular or linear polyribonucleotide, and a nucleotide spacer sequence that separates the termination element from a first translation initiation sequence of an expression succeeding the first expression sequence. In some examples, the first stagger element of the first expression sequence is upstream of (5′ to) a first translation initiation sequence of the expression succeeding the first expression sequence in the circular or linear polyribonucleotide. In some cases, the first expression sequence and the expression sequence succeeding the first expression sequence are two separate expression sequences in the circular or linear polyribonucleotide. The distance between the first stagger element and the first translation initiation sequence can enable continuous translation of the first expression sequence and its succeeding expression sequence. In some embodiments, the first stagger element includes a termination element and separates an expression product of the first expression sequence from an expression product of its succeeding expression sequences, thereby creating discrete expression products. In some cases, the circular or linear polyribonucleotide including the first stagger element upstream of the first translation initiation sequence of the succeeding sequence in the circular or linear polyribonucleotide is continuously translated, while a corresponding circular or linear polyribonucleotide including a stagger element of a second expression sequence that is upstream of a second translation initiation sequence of an expression sequence succeeding the second expression sequence is not continuously translated. In some cases, there is only one expression sequence in the circular or linear polyribonucleotide, and the first expression sequence and its succeeding expression sequence are the same expression sequence. In some exemplary circular or linear polyribonucleotides, a stagger element includes a first termination element of a first expression sequence in the circular or linear polyribonucleotide, and a nucleotide spacer sequence that separates the termination element from a downstream translation initiation sequence. In some such examples, the first stagger element is upstream of (5′ to) a first translation initiation sequence of the first expression sequence in the circular or linear polyribonucleotide. In some cases, the distance between the first stagger element and the first translation initiation sequence enables continuous translation of the first expression sequence and any succeeding expression sequences. In some embodiments, the first stagger element separates one round expression product of the first expression sequence from the next round expression product of the first expression sequences, thereby creating discrete expression products. In some cases, the circular or linear polyribonucleotide including the first stagger element upstream of the first translation initiation sequence of the first expression sequence in the circular or linear polyribonucleotide is continuously translated, while a corresponding circular or linear polyribonucleotide including a stagger element upstream of a second translation initiation sequence of a second expression sequence in the corresponding circular or linear polyribonucleotide is not continuously translated. In some cases, the distance between the second stagger element and the second translation initiation sequence is at least 2×, 3×, 4×, 5×, 6×, 7×, 8×, 9×, or 10× greater in the corresponding circular or linear polyribonucleotide than a distance between the first stagger element and the first translation initiation in the circular or linear polyribonucleotide. In some cases, the distance between the first stagger element and the first translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater. In some embodiments, the distance between the second stagger element and the second translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater than the distance between the first stagger element and the first translation initiation. In some embodiments, the circular or linear polyribonucleotide includes more than one expression sequence.

In some embodiments, a circular or linear polyribonucleotide includes at least one cleavage sequence. In some embodiments, the cleavage sequence is adjacent to an expression sequence. In some embodiments, the cleavage sequence is between two expression sequences. In some embodiments, cleavage sequence is included in an expression sequence. In some embodiments, the circular or linear polyribonucleotide includes between 2 and 10 cleavage sequences. In some embodiments, the circular or linear polyribonucleotide includes between 2 and 5 cleavage sequences. In some embodiments, the multiple cleavage sequences are between multiple expression sequences; for example, a circular or linear polyribonucleotide may include three expression sequences two cleavage sequences such that there is a cleavage sequence in between each expression sequence. In some embodiments, the circular or linear polyribonucleotide includes a cleavage sequence, such as in an immolating circRNA or cleavable circRNA or self-cleaving circRNA. In some embodiments, the circular or linear polyribonucleotide includes two or more cleavage sequences, leading to separation of the circular or linear polyribonucleotide into multiple products, e.g., miRNAs, linear RNAs, smaller circular or linear polyribonucleotide, etc.

In some embodiments, a cleavage sequence includes a ribozyme RNA sequence. A ribozyme (from ribonucleic acid enzyme, also called RNA enzyme or catalytic RNA) is an RNA molecule that catalyzes a chemical reaction. Many natural ribozymes catalyze either the hydrolysis of one of their own phosphodiester bonds, or the hydrolysis of bonds in other RNA, but they have also been found to catalyze the aminotransferase activity of the ribosome. Catalytic RNA can be “evolved” by in vitro methods. Similar to riboswitch activity discussed above, ribozymes and their reaction products can regulate gene expression. In some embodiments, a catalytic RNA or ribozyme can be placed within a larger non-coding RNA such that the ribozyme is present at many copies within the cell for the purposes of chemical transformation of a molecule from a bulk volume. In some embodiments, aptamers and ribozymes can both be encoded in the same non-coding RNA.

In some embodiments, the cleavage sequence encodes a cleavable polypeptide linker. For example, a polyribonucleotide may encode two or more immunogens, e.g., where the two or more immunogens are encoded by a single open-reading frame (ORF). For example, two or more immunogens may be encoded by a single open-reading frame, the expression of which is controlled by an IRES. In some embodiments, the ORF further encodes a polypeptide linker, e.g., such that the expression product of the ORF encodes two or more immunogens each separated by a sequence encoding a polypeptide linker (e.g., a linker of 5-200, 5 to 100, 5 to 50, 5 to 20, 50 to 100, or 50 to 200 amino acids). The polypeptide linker may include a cleavage site, for example, a cleavage site recognized and cleaved by a protease (e.g., an endogenous protease in a subject following administration of the polyribonucleotide to that subject). In such embodiments, a single expression product including the amino acid sequence of two or more immunogens is cleaved upon expression, such that the two or more immunogens are separated following expression. Exemplary protease cleavage sites are known to those of skill in the art, for example, amino acid sequences that act as protease cleavage sites recognized by a metalloproteinase (e.g., a matrix metalloproteinase (MMP), such as any one or more of MMPs 1-28), a disintegrin and metalloproteinase (ADAM, such as any one or more of ADAMs 2, 7-12, 15, 17-23, 28-30 and 33), a serine protease, urokinase-type plasminogen activator, matriptase, a cysteine protease, an aspartic protease, or a cathepsin protease. In some embodiments, the protease is MMP9 or MMP2. In some embodiments, the protease is matriptase.

In some embodiments, a circular or linear polyribonucleotide described herein is an immolating circular or linear polyribonucleotide, a cleavable circular or linear polyribonucleotide, or a self-cleaving circular or linear polyribonucleotide. A circular or linear polyribonucleotide can deliver cellular components including, for example, RNA, lncRNA, lincRNA, miRNA, tRNA, rRNA, snoRNA, ncRNA, siRNA, or shRNA. In some embodiments, a circular or linear polyribonucleotide includes miRNA separated by (i) self-cleavable elements; (ii) cleavage recruitment sites; (iii) degradable linkers; (iv) chemical linkers; and/or (v) spacer sequences. In some embodiments, circRNA includes siRNA separated by (i) self-cleavable elements; (ii) cleavage recruitment sites (e.g., ADAR); (iii) degradable linkers (e.g., glycerol); (iv) chemical linkers; and/or (v) spacer sequences. Non-limiting examples of self-cleavable elements include hammerhead, splicing element, hairpin, hepatitis delta virus (HDV), Varkud Satellite (VS), and g/mS ribozymes.

In some embodiments, the circular polyribonucleotide includes at least one stagger element adjacent to an expression sequence. In some embodiments, the circular polyribonucleotide includes a stagger element adjacent to each expression sequence. In some embodiments, the stagger element is present on one or both sides of each expression sequence, leading to separation of the expression products, e.g., peptide(s) and/or polypeptide(s). In some embodiments, the stagger element is a portion of the one or more expression sequences. In some embodiments, the circular polyribonucleotide comprises one or more expression sequences, and each of the one or more expression sequences is separated from a succeeding expression sequence by a stagger element on the circular polyribonucleotide. In some embodiments, the stagger element prevents generation of a single polypeptide (a) from two rounds of translation of a single expression sequence or (b) from one or more rounds of translation of two or more expression sequences. In some embodiments, the stagger element is a sequence separate from the one or more expression sequences. In some embodiments, the stagger element comprises a portion of an expression sequence of the one or more expression sequences.

Examples of stagger elements are described in paragraphs [0172]-[0175] of WO2019/118919, which is hereby incorporated by reference in its entirety.

### Translation Initiation Sequences

In some embodiments, a circular or linear polyribonucleotide encodes an immunogen and includes a translation initiation sequence, e.g., a start codon. In some embodiments, the circular polyribonucleotide encodes an immunogen that produces the human polyclonal antibodies of interest and comprises a translation initiation sequence, e.g., a start codon. In some embodiments, the translation initiation sequence includes a Kozak or Shine-Dalgarno sequence. In some embodiments, the translation initiation sequence includes a Kozak sequence. In some embodiments, the circular or linear polyribonucleotide includes the translation initiation sequence, e.g., Kozak sequence, adjacent to an expression sequence. In some embodiments, the translation initiation sequence is a non-coding start codon. In some embodiments, the translation initiation sequence, e.g., Kozak sequence, is present on one or both sides of each expression sequence, leading to separation of the expression products. In some embodiments, the circular or linear polyribonucleotide includes at least one translation initiation sequence adjacent to an expression sequence. In some embodiments, the translation initiation sequence provides conformational flexibility to the circular or linear polyribonucleotide. In some embodiments, the translation initiation sequence is within a substantially single stranded region of the circular or linear polyribonucleotide. Further examples of translation initiation sequences are described in paragraphs [0163]-[0165] of International Patent Publication No. WO2019/118919, which is hereby incorporated by reference in its entirety.

The circular or linear polyribonucleotide may include more than 1 start codon such as, but not limited to, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 50, at least 60 or more than 60 start codons. Translation may initiate on the first start codon or may initiate downstream of the first start codon.

In some embodiments, a circular or linear polyribonucleotide may initiate at a codon which is not the first start codon, e.g., AUG. Translation of the circular or linear polyribonucleotide may initiate at an alternative translation initiation sequence, such as those described in [0164] of International Patent Publication No. WO2019/118919A1, which is incorporated herein by reference in its entirety.

In some embodiments, translation is initiated by eukaryotic initiation factor 4A (eIF4A) treatment with Rocaglates (translation is repressed by blocking 43S scanning, leading to premature, upstream translation initiation and reduced protein expression from transcripts bearing the RocA-eIF4A target sequence, see for example, nature.com/articles/naturel7978).

### Untranslated Regions

In some embodiments, a circular or linear polyribonucleotide includes untranslated regions (UTRs). UTRs of a genomic region including a gene may be transcribed but not translated. In some embodiments, a UTR may be included upstream of the translation initiation sequence of an expression sequence described herein. In some embodiments, a UTR may be included downstream of an expression sequence described herein. In some instances, one UTR for the first expression sequence is the same as or continuous with or overlapping with another UTR for a second expression sequence. In some embodiments, the intron is a human intron. In some embodiments, the intron is a full-length human intron, e.g., ZKSCAN1.

Exemplary untranslated regions are described in paragraphs [0197]-[201] of International Patent Publication No. WO2019/118919, which is hereby incorporated by reference in its entirety.

In some embodiments, a circular polyribonucleotide includes a poly-A sequence. Exemplary poly-A sequences are described in paragraphs [0202]-[0205] of International Patent Publication No. WO2019/118919, which is hereby incorporated by reference in its entirety. In some embodiments, a circular polyribonucleotide lacks a poly-A sequence.

In some embodiments, a circular or linear polyribonucleotide includes a UTR with one or more stretches of Adenosines and Uridines embedded within. These AU rich signatures may increase turnover rates of the expression product.

Introduction, removal, or modification of UTR AU rich elements (AREs) may be useful to modulate the stability, or immunogenicity (e.g., the level of one or more markers of an immune or inflammatory response) of the circular or linear polyribonucleotide. When engineering specific circular polyribonucleotides, one or more copies of an ARE may be introduced to the circular polyribonucleotide and the copies of an ARE may modulate translation and/or production of an expression product. Likewise, AREs may be identified and removed or engineered into the circular polyribonucleotide to modulate the intracellular stability and thus affect translation and production of the resultant protein.

It should be understood that any UTR from any gene may be incorporated into the respective flanking regions of the circular polyribonucleotide.

In some embodiments, a circular polyribonucleotide lacks a 5′-UTR and is competent for protein expression from its one or more expression sequences. In some embodiments, the circular or linear polyribonucleotide lacks a 3′-UTR and is competent for protein expression from its one or more expression sequences. In some embodiments, the circular or linear polyribonucleotide lacks a poly-A sequence and is competent for protein expression from its one or more expression sequences. In some embodiments, the circular or linear polyribonucleotide lacks a termination element and is competent for protein expression from its one or more expression sequences. In some embodiments, the circular or linear polyribonucleotide lacks an internal ribosomal entry site and is competent for protein expression from its one or more expression sequences. In some embodiments, the circular or linear polyribonucleotide lacks a cap and is competent for protein expression from its one or more expression sequences. In some embodiments, the circular or linear polyribonucleotide lacks a 5′-UTR, a 3′-UTR, and an IRES, and is competent for protein expression from its one or more expression sequences. In some embodiments, the circular or linear polyribonucleotide includes one or more of the following sequences: a sequence that encodes one or more miRNAs, a sequence that encodes one or more replication proteins, a sequence that encodes an exogenous gene, a sequence that encodes a therapeutic, a regulatory element (e.g., translation modulator, e.g., translation enhancer or suppressor), a translation initiation sequence, one or more regulatory nucleic acids that targets endogenous genes (e.g., siRNA, lncRNAs, shRNA), and a sequence that encodes a therapeutic mRNA or protein.

In some embodiments, a circular or linear polyribonucleotide lacks a 5′-UTR. In some embodiments, the circular polyribonucleotide lacks a 3′-UTR. In some embodiments, the circular polyribonucleotide lacks a poly-A sequence. In some embodiments, the circular or linear polyribonucleotide lacks a termination element. In some embodiments, the circular or linear polyribonucleotide lacks an internal ribosomal entry site. In some embodiments, the circular or linear polyribonucleotide lacks degradation susceptibility by exonucleases. In some embodiments, the fact that the circular polyribonucleotide lacks degradation susceptibility can mean that the circular polyribonucleotide is not degraded by an exonuclease, or only degraded in the presence of an exonuclease to a limited extent, e.g., that is comparable to or similar to in the absence of exonuclease. In some embodiments, the circular polyribonucleotide is not degraded by exonucleases. In some embodiments, the circular polyribonucleotide has reduced degradation when exposed to exonuclease. In some embodiments, the circular polyribonucleotide lacks binding to a cap-binding protein. In some embodiments, the circular polyribonucleotide lacks a 5′ cap.

### Termination Elements

In some embodiments, the polyribonucleotide described herein includes at least one termination element. In some embodiments, the polyribonucleotide includes a termination element operably linked to an expression sequence. In some embodiments, the polynucleotide lacks a termination element.

In some embodiments, the polyribonucleotide includes one or more expression sequences, and each expression sequence may or may not have a termination element. In some embodiments, the polyribonucleotide includes one or more expression sequences, and the expression sequences lack a termination element, such that the polyribonucleotide is continuously translated. Exclusion of a termination element may result in rolling circle translation or continuous expression of expression product.

In some embodiments, the circular polyribonucleotide includes one or more expression sequences, and each expression sequence may or may not have a termination element. In some embodiments, the circular polyribonucleotide includes one or more expression sequences, and the expression sequences lack a termination element, such that the circular polyribonucleotide is continuously translated. Exclusion of a termination element may result in rolling circle translation or continuous expression of expression product, e.g., peptides or polypeptides, due to lack of ribosome stalling or fall-off. In such an embodiment, rolling circle translation expresses a continuous expression product through each expression sequence. In some other embodiments, a termination element of an expression sequence can be part of a stagger element. In some embodiments, one or more expression sequences in the circular polyribonucleotide includes a termination element. However, rolling circle translation or expression of a succeeding (e.g., second, third, fourth, fifth, etc.) expression sequence in the circular polyribonucleotide is performed. In such instances, the expression product may fall off the ribosome when the ribosome encounters the termination element, e.g., a stop codon, and terminates translation. In some embodiments, translation is terminated while the ribosome, e.g., at least one subunit of the ribosome, remains in contact with the circular polyribonucleotide.

In some embodiments, the circular polyribonucleotide includes a termination element at the end of one or more expression sequences. In some embodiments, one or more expression sequences includes two or more termination elements in succession. In such embodiments, translation is terminated and rolling circle translation is terminated. In some embodiments, the ribosome completely disengages with the circular polyribonucleotide. In some such embodiments, production of a succeeding (e.g., second, third, fourth, fifth, etc.) expression sequence in the circular polyribonucleotide may require the ribosome to reengage with the circular polyribonucleotide prior to initiation of translation. Generally, termination elements include an in-frame nucleotide triplet that signals termination of translation (e.g., UAA, UGA, UAG). In some embodiments, one or more termination elements in the circular polyribonucleotide are frame-shifted termination elements, such as but not limited to, off-frame or −1 and +1 shifted reading frames (e.g., hidden stop) that may terminate translation. Frame-shifted termination elements include nucleotide triples, TAA, TAG, and TGA that appear in the second and third reading frames of an expression sequence. Frame-shifted termination elements may be important in preventing misreads of mRNA, which is often detrimental to the cell. In some embodiments, the termination element is a stop codon.

In some embodiments, an expression sequence includes a poly-A sequence (e.g., at the 3′ end of an expression sequence, for example 3′ to a termination element). In some embodiments, the length of a poly-A sequence is greater than 10 nucleotides in length. In one embodiment, the poly-A sequence is greater than 15 nucleotides in length (e.g., at least or greater than about 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the poly-A sequence is designed according to the descriptions of the poly-A sequence in [0202]-[0204] of International Patent Publication No. WO2019/118919A1, which is incorporated herein by reference in its entirety. In some embodiments, the expression sequence lacks a poly-A sequence (e.g., at the 3′ end of an expression sequence).

In some embodiments, a circular polyribonucleotide includes a polyA, lacks a polyA, or has a modified polyA to modulate one or more characteristics of the circular polyribonucleotide. In some embodiments, the circular polyribonucleotide lacking a polyA or having modified polyA improves one or more functional characteristics, e.g., immunogenicity (e.g., the level of one or more marker of an immune or inflammatory response), half-life, and/or expression efficiency.

Further examples of termination elements are described in paragraphs [0169]-[0170] of International Patent Publication No. WO2019/118919, which is hereby incorporated by reference in its entirety.

### Spacer Sequences

In some embodiments, a polyribonucleotide described herein includes a spacer sequence. In some embodiments, a polyribonucleotide described herein includes one or more spacer sequences. A spacer refers to any contiguous nucleotide sequence (e.g., of one or more nucleotides) that provides distance or flexibility between two adjacent polynucleotide regions. Spacers may be present in between any of the nucleic acid elements described herein. Spacer may also be present within a nucleic acid element described herein.

The spacer may be, e.g., at least 5 (e.g., at least 10, at least 15, at least 20) ribonucleotides in length. In some embodiments, each spacer region is at least 5 (e.g., at least 10, at least 15, at least 20) ribonucleotides in length. Each spacer region may be, e.g., from 5 to 500 (e.g., 10, 20, 30, 40, 50, 60, 70, 80, 90, 100, 150, 200, 250, 300, 350, 400, 450, or 500) ribonucleotides in length. The first spacer region, the second spacer region, or the first spacer region and the second spacer region may include a polyA sequence. The first spacer region, the second spacer region, or the first spacer region and the second spacer region may include a polyA-C sequence. In some embodiments, the first spacer region, the second spacer region, or the first spacer region and the second spacer region includes a polyA-G sequence. In some embodiments, the first spacer region, the second spacer region, or the first spacer region and the second spacer region includes a polyA-T sequence. In some embodiments, the first spacer region, the second spacer region, or the first spacer region and the second spacer region includes a random sequence.

In some embodiments, the spacer sequence can be, for example, at least 10 nucleotides in length, at least 15 nucleotides in length, or at least 30 nucleotides in length. In some embodiments, the spacer sequence is at least 7, 8, 9, 10, 11, 12, 13, 14, 15, 16, 17, 18, 19, 20, 25 or 30 nucleotides in length. In some embodiments, the spacer sequence is no more than 100, 90, 80, 70, 60, 50, 45, 40, 35 or 30 nucleotides in length. In some embodiments the spacer sequence is from 20 to 50 nucleotides in length. In certain embodiments, the spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length.

The spacer sequences can be polyA sequences, polyA-C sequences, polyC sequences, or poly-U sequences.

In some embodiments, the spacer sequences can be polyA-T, polyA-C, polyA-G, or a random sequence.

Exemplary spacer sequences are described in paragraphs [0293]-[0302] of International Patent Publication No. WO2019/118919, which is hereby incorporated by reference in its entirety.

### Modifications

A polyribonucleotide may include one or more substitutions, insertions and/or additions, deletions, and covalent modifications with respect to reference sequences, in particular, the parent polyribonucleotide, are included within the scope of this disclosure.

In some embodiments, a polyribonucleotide includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27: 196-197). In some embodiments, the first isolated nucleic acid includes messenger RNA (mRNA). In some embodiments, the polyribonucleotide includes at least one nucleoside selected from the group such as those described in [0311] of International Patent Publication No. WO2019/118919A1, which is incorporated herein by reference in its entirety.

A polyribonucleotide may include any useful modification, such as to the sugar, the nucleobase, or the internucleoside linkage (e.g., to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, a polyribonucleotide includes at least one N(6)methyladenosine (m6A) modification to increase translation efficiency. In some embodiments, the m6A modification can reduce immunogenicity (e.g., reduce the level of one or more marker of an immune or inflammatory response) of the polyribonucleotide.

In some embodiments, a modification may include a chemical or cellular induced modification. For example, some non-limiting examples of intracellular RNA modifications are described by Lewis and Pan in “RNA modifications and structures cooperate to guide RNA-protein interactions” from Nat Reviews Mol Cell Biol, 2017, 18:202-210.

In some embodiments, chemical modifications to the ribonucleotides of a polyribonucleotide may enhance immune evasion. The polyribonucleotide may be synthesized and/or modified by methods well established in the art, such as those described in CURRENT PROTOCOLS IN NUCLEIC ACID CHEMISTRY, Beaucage, S. L. et al. (Eds.), John Wiley & Sons, Inc., New York, NY, USA, which is hereby incorporated herein by reference. Modifications include, for example, end modifications, e.g., 5′ end modifications (phosphorylation (mono-, di- and tri-), conjugation, inverted linkages, etc.), 3′ end modifications (conjugation, DNA nucleotides, inverted linkages, etc.), base modifications (e.g., replacement with stabilizing bases, destabilizing bases, or bases that base pair with an expanded repertoire of partners), removal of bases (abasic nucleotides), or conjugated bases. The modified ribonucleotide bases may also include 5-methylcytidine and pseudouridine. In some embodiments, base modifications may modulate expression, immune response, stability, subcellular localization, to name a few functional effects, of the polyribonucleotide. In some embodiments, the modification includes a bi-orthogonal nucleotide, e.g., an unnatural base. See for example, Kimoto et al, Chem Commun (Camb), 2017, 53:12309, DOI: 10.1039/c7cc06661a, which is hereby incorporated by reference.

In some embodiments, sugar modifications (e.g., at the 2′ position or 4′ position) or replacement of the sugar one or more ribonucleotides of the polyribonucleotide may, as well as backbone modifications, include modification or replacement of the phosphodiester linkages. Specific examples of polyribonucleotide include, but are not limited to, polyribonucleotide including modified backbones or no natural internucleoside linkages such as internucleoside modifications, including modification or replacement of the phosphodiester linkages. Polyribonucleotides having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, the polyribonucleotide will include ribonucleotides with a phosphorus atom in its internucleoside backbone.

Modified polyribonucleotide backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the polyribonucleotide may be negatively or positively charged.

The modified nucleotides, which may be incorporated into the polyribonucleotide, can be modified at the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylenephosphonates).

The a-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment. Phosphorothioate linked to the polyribonucleotide is expected to reduce the innate immune response through weaker binding/activation of cellular innate immune molecules.

In specific embodiments, a modified nucleoside includes an alpha-thio-nucleoside (e.g., 5′-O—(I-thiophosphate)-adenosine, 5′-O—(I-thiophosphate)-cytidine (a-thio-cytidine), 5′-O—(I-thiophosphate)-guanosine, 5′-O—(I-thiophosphate)-uridine, or 5′-O-(1-thiophosphate)-pseudouridine).

Other internucleoside linkages that may be employed according to the present disclosure, including internucleoside linkages which do not contain a phosphorous atom, are described herein.

In some embodiments, a polyribonucleotide may include one or more cytotoxic nucleosides. For example, cytotoxic nucleosides may be incorporated into polyribonucleotide, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, I-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-I-(tetrahydrofuran-2-yl)pyrimidine-2,4(IH,3H)-dione), troxacitabine, tezacitabine, 2′-deoxy-2′-methylidenecytidine (DMDC), and 6-mercaptopurine. Additional examples include fludarabine phosphate, N4-behenoyl-I-beta-D-arabinofuranosylcytosine, N4-octadecyl-1-beta-D-arabinofuranosylcytosine, N4-palmitoyl-I-(2-C-cyano-2-deoxy-beta-D-arabino-pentofuranosyl) cytosine, and P-4055 (cytarabine 5′-elaidic acid ester).

A polyribonucleotide may or may not be uniformly modified along the entire length of the molecule. For example, one or more or all types of nucleotides (e.g., naturally occurring nucleotides, purine or pyrimidine, or any one or more or all of A, G, U, C, 1, pU) may or may not be uniformly modified in the polyribonucleotide, or in a given predetermined sequence region thereof. In some embodiments, the polyribonucleotide includes a pseudouridine. In some embodiments, the polyribonucleotide includes an inosine, which may aid in the immune system characterizing the polyribonucleotide as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

In some embodiments, all nucleotides in a polyribonucleotide (or in a given sequence region thereof) are modified. In some embodiments, the modification may include an m6A, which may augment expression; an inosine, which may attenuate an immune response; pseudouridine, which may increase RNA stability, or translational readthrough (stagger element), an m5C, which may increase stability; and a 2,2,7-trimethylguanosine, which aids subcellular translocation (e.g., nuclear localization).

Different sugar modifications, nucleotide modifications, and/or internucleoside linkages (e.g., backbone structures) may exist at various positions in a polyribonucleotide. One of ordinary skill in the art will appreciate that the nucleotide analogs or other modification(s) may be located at any position(s) of the polyribonucleotide, such that the function of the polyribonucleotide is not substantially decreased. A modification may also be a non-coding region modification. The polyribonucleotide may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

### Multimerization

In certain embodiments, the circular polyribonucleotide may include a multimerization domain. For example, a circular polyribonucleotide may encode a first polypeptide that is an immunogen (e.g., a coronavirus immunogen) and a second polypeptide that is a multimerization domain. For example, a multimerization domain may be encoded in the same open reading frame as an immunogen (e.g., a coronavirus immunogen) and expressed as fusion protein with the immunogen. In some embodiments, the circular polyribonucleotide may encode two or more immunogens, and each immunogen may optionally be fused to a multimerization domain. The multimerization domain may promote the formation of immunogen complexes (e.g., a complex including a plurality of immunogens).

Multimerization of the encoded immunogen may be beneficial for the induction of an immune response. Fusion of the immunogen to one or more multimerization elements (e.g., dimerization elements, trimerization elements, tetramerization elements, and oligomerization elements) may lead to the formation of a multimeric immunogen complex (e.g., formation of a multimeric immunogen complex following expression in an immunized subject). In some embodiments, formation of a multimeric immunogen complex increases immunogenicity of the immunogen. For example, formation of a multimeric immunogen complex may increase immunogenicity of the immunogen by mimicking an infection with an exogenous pathogen (e.g., a virus) where a plurality of potential immunogens is commonly located at the envelope of the pathogen (e.g., hemagglutinin (HA) immunogen of the influenza virus). In some embodiments, the multimerization complex includes at least 2, 3, 4, 6, 8, 10, 15, 20, 25, 30, 35, 40, 45, 50, 55, 60, 70, 80, 90, or 100 immunogens. In some embodiments, the immunogen complex includes 2 to 10, 2 to 50, 2 to 100, 5 to 10, 5 to 15, 5 to 20, 5 to 50, 5 to 100, 10 to 20, 10 to 30, 10 to 40, 10 to 50, 10 to 60, 10 to 100, 20 to 50 or 20 to 100 immunogens. In some embodiments, the immunogen complex comprises 6 copies of the immunogen (e.g., the circular polyribonucleotide encodes an immunogen-foldon-immunogen fusion protein). In some embodiments, the immunogen complex comprises 24 copies of the immunogen (e.g., the circular polyribonucleotide encodes an immunogen-ferritin fusion protein). In some embodiments, the immunogen complex comprises 60 copies of the immunogen (e.g., the circular polyribonucleotide encodes an immunogen-AaLS fusion protein or encodes immunogen-β-annulus peptide).

When used in combination with a polypeptide immunogen of interest in the context of the present disclosure, such multimerization elements can be placed N-terminal or C-terminal to the polypeptide of interest. On nucleic acid level, the coding sequence for such multimerization element is typically placed in the same reading frame, 5′ or 3′ to the coding sequence for the polypeptide or protein of interest.

The multimerization domain may have between 10 and 500 amino acid residues (e.g., between 10 and 450, 10 and 400, 10 and 350, 10 and 300, 10 and 250, 10 and 200, 10 and 150, 10 and 100, 10 and 50, 50 and 500, 100 and 500, 150 and 500, 200 and 500, 250 and 500, 300 and 500, 350 and 500, 400 and 500, and 450 and 500 residues). In some embodiments, the multimerization domain may include between 20 and 2500 amino acid residues (e.g., between 20 and 250, 20 and 225, 20 and 200, 20 and 175, 20 and 150, 20 and 150, 20 and 125, 20 and 100, 20 and 75, 20 and 50, 50 and 250, 75 and 250, 100 and 250, 125 and 250, 150 and 250, 175 and 250, 200 and 250, and 225 and 250 residues).

In some embodiments, an immunogen fused to the multimerization domain is at least 2-fold, 5-fold, or 10-fold more immunogenic than the immunogen (e.g., in a human subject). In some embodiments, the immunogen fused to a multimerization domain is at least 20%, 30%, 40%, 50%, 60%, 70%, 80%, 90%, 100%, 200%, 300%, 400%, or 500% more immunogenic (e.g., in a human subject) than the immunogen not fused to a multimerization domain.

Particular multimerization elements are oligomerization elements, tetramerization elements, trimerization elements or dimerization elements. Dimerization elements may be selected from e.g., dimerization elements/domains of heat shock proteins, immunoglobulin Fc domains and leucine zippers (dimerization domains of the basic region leucine zipper class of transcription factors). Trimerization and tetramerization elements may be selected from e.g., engineered leucine zippers (engineered a-helical coiled coil peptide that adopt a parallel trimeric state), fibritin foldon domain from enterobacteria phage T4, GCN4pll, CCN4-pLI, and p53. In some embodiments, the circular polyribonucleotide includes a T4 foldon domain. In particular embodiments, the T4 foldon domain has an amino acid sequence that is at least 95% identical to GYIPEAPRDGQAYVRKDGEWVLLSTFL (SEQ ID NO: 204). In some embodiments, the T4 foldon has an amino acid sequence of SEQ ID NO: 204. In some embodiments, the multimerization domain is a β-annulus peptide (see, Matsuura et al. (2010), ANGEW. CHEM. INT. ED., 49: 9662-65). In some embodiments, the β-annulus peptide has an amino acid sequence of INHVGGTGGAIMAPVAVTRQLVGS (SEQ ID NO: 205), where the C-terminal Serine residue is optionally present or absent or has an amino acid sequence that is at least 95% identical to SEQ ID NO: 205. In some embodiments, the circular polyribonucleotide includes an AaLS peptide. In particular embodiments, the AaLS peptide has an amino acid sequence that is at least 95% identical to TDILGKYVINYLNKLKKKEDIFKEFLKW (SEQ ID NO: 282). In some embodiments, the AaLS peptide has an amino acid sequence of SEQ ID NO: 282.

Oligomerization elements may be selected from e.g., ferritin, surfactant D, oligomerization domains of phosphoproteins of paramyxoviruses, complement inhibitor C4 binding protein (C4 bp) oligomerization domains, Viral infectivity factor (Vif) oligomerization domain, sterile alpha motif (SAM) domain, and von Willebrand factor type D domain.

Ferritin forms oligomers and is a highly conserved protein found in all animals, bacteria, and plants. Ferritin is a protein that spontaneously forms nanoparticles of 24 identical subunits. Ferritin-immunogen fusion constructs potentially form oligomeric aggregates or “clusters” of immunogens that may enhance the immune response. In some embodiments, the circular polyribonucleotide includes a ferritin domain. In some embodiments, the circular polyribonucleotide includes a ferritin domain having the amino acid sequence of:
DIIKLLNEQVNKEMNSSNLYMSMSSWCYTHSLDGAGLFLFDHAAEEYEHAKKLIVFLNEN NVPVQLTSISAPEHKFESLTQIFQKAYEHEQHISESINNIVDHAIKGKDHATFNFLQWYVS EQHEEEVLFKDILDKIELIGNENHGLYLADQYVKGIAKSRKS (SEQ ID NO: 207).

Surfactant D protein (SPD) is a hydrophilic glycoprotein that spontaneously self-assembles to form oligomers. An SPD-immunogen fusion constructs may form oligomeric aggregates or “clusters” of immunogens that may enhance the immune response.

Phosphoprotein of paramyxoviruses (negative sense RNA viruses) functions as a transcriptional transactivator of the viral polymerase. Oligomerization of the phosphoprotein is critical for viral genome replication. A phosphoprotein-immunogen fusion constructs may form oligomeric aggregates or “clusters” of immunogens that may enhance the immune response.

Complement inhibitor C4 binding Protein (C4 bp) may also be used as a fusion partner to generate oligomeric immunogen aggregates. The C-terminal domain of C4 bp (57 amino acid residues in humans and 54 amino acid residues in mice) is both necessary and sufficient for the oligomerization of C4 bp or other polypeptides fused to it. A C4 bp-immunogen fusion constructs may form oligomeric aggregates or “clusters” of immunogens that may enhance the immune response. Viral infectivity factor (Vif) multimerization domain has been shown to form oligomers both in vitro and in vivo. The oligomerization of Vif involves a sequence mapping between residues 1 51 to 1 64 in the C-terminal domain, the 1 61 PPLP1 64 motif (for human HIV-1: TPKKIKPPLP (SEQ ID NO: 327). A Vif-immunogen fusion constructs may form oligomeric aggregates or “clusters” of immunogens that may enhance the immune response.

The sterile alpha motif (SAM) domain is a protein interaction module present in a wide variety of proteins involved in many biological processes. The SAM domain that spreads over around 70 residues is found in diverse eukaryotic organisms. SAM domains have been shown to homo- and hetero-oligomerise, forming multiple self-association oligomeric architectures. A SAM-immunogen fusion constructs may form oligomeric aggregates or “clusters” of immunogens that may enhance the immune response. von Willebrand factor (vWF) contains several type D domains: D1 and D2 are present within the N-terminal propeptide whereas the remaining D domains are required for oligomerization. The vWF domain is found in various plasma proteins: complement factors B, C2, C 3 and CR4; the Integrins (I-domains); collagen types VI, VII, XII and XIV; and other extracellular proteins. A vWF-immunogen fusion constructs may form oligomeric aggregates or “clusters” of immunogens that may enhance the immune response.

In some embodiments, the circular polyribonucleotide may include one or more multimerization domains. For example, the circular polyribonucleotide may include 2, 3, 4, 5, 6, 7, 8, 9, or 10 multimerization domains. In some embodiments, the circular polyribonucleotide includes two multimerization domains. Two or more multimerization domains may be adjacent to one another. Alternatively, two or more multimerization domains may be separated by one or more other elements. For example, two multimerization domains may be separated by an immunogen. In particular embodiments, the circular polyribonucleotide includes a ferritin domain and a T4 foldon domain. The ferritin and T4 foldon domain may be linked by a Gly-Ser linker. In some embodiments, the ferritin domain linked to the T4 foldon domain has an amino acid sequence of:

(SEQ ID NO: 206)
PGSGYIPEAPRDGQAYVRKDGEWVLLSTFLSGRSGGDIIKLLNEQVNKE
MNSSNLYMSMSSWCYTHSLDGAGLFLFDHAAEEYEHAKKLIVFLNENNV
PVQLTSISAPEHKFESLTQIFQKAYEHEQHISESINNIVDHAIKGKDHA
TFNFLQWYVSEQHEEEVLFKDILDKIELIGNENHGLYLADQYVKGIAKS
RKS.

In some embodiments, the multimerization domain is a lumazine synthase domain. Lumazine synthase may assemble into a complex including 60 copies of the lumazine synthase domain, where each lumazine synthase domain may be fused to one or more immunogens. In some embodiments, the lumazine synthase domain includes an amino acid sequence of any of SEQ ID NOs: 208-209, 325, and 328-329 or an amino acid sequence having a least 95% sequence identity with any one of SEQ ID NOs: 208-209, 325, and 328-329.

SEQ ID NO: 328
MQIYEGKLTAEGLRFGIVASRFNHALVDRLVEGAIDAIVRHGGREEDIT
LVRVPGSWEIPVAAGELARKEDIDAVIAIGVLIRGATPHFDYIASEVSK
GLADLSLELRKPITFGVITADTLEQAIERAGTKHGNKGWEAALSAIEMA
NLFKSLR
SEQ ID NO: 329
QIYEGKLTAEGLRFGIVASRFNHALVDRLVEGCIDCIVRHGGREEDITL
VRVPGSWEIPVAAGELARKEDIDAVIAIGVLIRGATPHFDYIASEVSKG
LANLSLELRKPITFGVITADTLEQAIERAGTKHGNKCWEAALSAIEMAN
LFKSLR
SEQ ID NO: 208
QIYEGKLTAEGLRFGIVASRFNHALVDRLVEGAIDAIVRHGGREEDITL
VRVPGSWEIPVAAGELARKENISAVIAIGVLIRGATPHFDYIASEVSKG
LADLSLELRKPITFGVITADTLEQAIERAGTKHGNKGWEAALSAIEMAN
LFKSLR
SEQ ID NO: 209
QIYEGKLTAEGLRFGIVASRFNHALVDRLVEGAIDCIVRHGGREEDITL
VRVPGSWEIPVAAGELARKEDIDAVIAIGVLIRGATPHFDYIASEVSKG
LADLSLELRKPITFGVITADTLEQAIERAGTKHGNKGWEAALSAIEMAN
LFKSLR
SEQ ID NO: 325
MQIYEGKLTAEGLRFGIVASRFNHALVDRLVEGAIDCIVRHGGREEDIT
LVRVPGSWEIPVAAGELARKEDIDAVIAIGVLIRGATPHFDYIASEVSK
GLANLSLELRKPITFGVITADTLEQAIERAGTKHGNKGWEAALSAIEMA
NLFKSLR

Lumazine synthase domains are provided with one or more cysteine substitutions to introduce non-native disulfide bond(s) that stabilize the lumazine synthase complex formed from self-assembled subunits. In some embodiments, the non-native disulfide bond(s) are introduced with L121C-K131C, L121CG-K131C, L121GC-K131C, K7C-R40C, 13C-L50C, 182C-K131CG, E5C-R52C, or E95C-A101C substitutions, or a combination thereof (such as 13C-L50C and 182C-K131CG; E5C-R52C and 182C-K131CG; or E95C-A101C and 182C-K131CG). The residues numbering is with reference to the lumazine synthase subunit set forth as SEQ ID NO: 328. Non-limiting examples include:

(L121C-K131C)
SEQ ID NO: 210
QIYEGKLTAEGLRFGIVASRFNHALVDRLVEGAIDAIVRHGGREEDITL
VRVPGSWEIPVAAGELARKENISAVIAIGVLIRGATPHFDYIASEVSKG
LADLSLELRKPITFGVITADTCEQAIERAGTcHGNKGWEAALSAIEMAN
LFKSLR
(L121CG-K131C)
SEQ ID NO: 211
QIYEGKLTAEGLRFGIVASRFNHALVDRLVEGAIDAIVRHGGREEDITL
VRVPGSWEIPVAAGELARKENISAVIAIGVLIRGATPHFDYIASEVSKG
LADLSLELRKPITFGVITADTcCfEQAIERAGTcHGNKGWEAALSAIEM
ANLFKSLR
(L121GC-K131C)
SEQ ID NO: 212
QIYEGKLTAEGLRFGIVASRFNHALVDRLVEGAIDAIVRHGGREEDITL
VRVPGSWEIPVAAGELARKENISAVIAIGVLIRGATPHFDYIASEVSKG
LADLSLELRKPITFGVITADTCfcEQAIERAGTcHGNKGWEAALSAIEM
ANLFKSLR
(K7C-R40C)
SEQ ID NO: 213
QIYEGCLTAEGLRFGIVASRFNHALVDRLVEGAIDAIVCHGGREEDITL
VRVPGSWEIPVAAGELARKENISAVIAIGVLIRGATPHFDYIASEVSKG
LADLSLELRKPITFGVITADTLEQAIERAGTKHGNKGWEAALSAIEMAN
LFKSLR
(I3C-L50C, I82C-K131CG)
SEQ ID NO: 214
QCYEGKLTAEGLRFGIVASRFNHALVDRLVEGAIDCIVRHGGREEDITC
VRVPGSWEIPVAAGELARKEDIDAVIAIGVLCRGATPHFDYIASEVSKG
LADLSLELRKPITFGVITADTLEQAIERAGTCGHGNKGWEAALSAIEMA
NLFKSLR
(E5C-R52C, I82C-K131CG)
SEQ ID NO: 215
QIYCGKLTAEGLRFGIVASRFNHALVDRLVEGAIDCIVRHGGREEDITL
VCVPGSWEIPVAAGELARKEDIDAVIAIGVLCRGATPHFDYIASEVSKG
LADLSLELRKPITFGVITADTLEQAIERAGTCGHGNKGWEAALSAIEMA
NLFKSLR
(E95C-A101C, I82C-K131CG)
SEQ ID NO: 216
QIYEGKLTAEGLRFGIVASRFNHALVDRLVEGAIDCIVRHGGREEDITL
VRVPGSWEIPVAAGELARKEDIDAVIAIGVLCRGATPHFDYIASCVSKG
LCDLSLELRKPITFGVITADTLEQAIERAGTCGHGNKGWEAALSAIEMA
NLFKSLR

Various methods of multimerization of polypeptides are described International Publication No. WO2020/061564, page 25, line 1 through page 26 line 20 which is herein incorporated by reference.

In some embodiments, the multimerization domain is a riboflavin synthase domain. For example, the riboflavin synthase domain may have an amino acid sequence having a least 95% sequence identity TDILGKYVINYLNKLKKKEDIFKEFLKW (SEQ ID NO: 326). In some embodiments, the riboflavin synthase domain may have an amino acid sequence of SEQ ID NO: 326.

Suitable multimerization domains may be selected, for example, from the list of amino acid sequences according to SEQ ID NOs: 1116-1167 of the international patent application WO2017/081082, or fragments or variants of these sequences.

### Production Methods

The disclosure provides methods for producing circular polyribonucleotides, including, e.g., recombinant technology or chemical synthesis. For example, a DNA molecule used to produce an RNA circle can include a DNA sequence of a naturally occurring nucleic acid sequence, a modified version thereof, or a DNA sequence encoding a synthetic polypeptide not normally found in nature (e.g., chimeric molecules or fusion proteins). DNA and RNA molecules can be modified using a variety of techniques including, but not limited to, classic mutagenesis techniques and recombinant techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification or mutagenesis of selected regions of a nucleic acid sequence, synthesis of oligonucleotide mixtures and ligation of mixture groups to “build” a mixture of nucleic acid molecules and combinations thereof.

The circular polyribonucleotides may be prepared according to any available technique, including, but not limited to chemical synthesis and enzymatic synthesis. In some embodiments, a linear primary construct or linear RNA may be cyclized or concatenated to create a circRNA described herein. The mechanism of cyclization or concatenation may occur through methods such as, e.g., chemical, enzymatic, splint ligation, or ribozyme-catalyzed methods. The newly formed 5′-3′ linkage may be an intramolecular linkage or an intermolecular linkage. For example, a splint ligase, such as a SplintR® ligase, can be used for splint ligation. According to this method, a single stranded polynucleotide (splint), such as a single-stranded DNA or RNA, can be designed to hybridize with both termini of a linear polyribonucleotide, so that the two termini can be juxtaposed upon hybridization with the single-stranded splint. Splint ligase can thus catalyze the ligation of the juxtaposed two termini of the linear polyribonucleotide, generating a circRNA. In some embodiments, a DNA or RNA ligase may be used in the synthesis of the circular polynucleotides. As a non-limiting example, the ligase may be a circ ligase or circular ligase.

In another example, either the 5′ or 3′ end of the linear polyribonucleotide can encode a ligase ribozyme sequence such that during in vitro transcription, the resultant linear circRNA includes an active ribozyme sequence capable of ligating the 5′ end of the linear polyribonucleotide to the 3′ end of the linear polyribonucleotide. The ligase ribozyme may be derived from the Group I Intron, Hepatitis Delta Virus, Hairpin ribozyme or may be selected by SELEX (systematic evolution of ligands by exponential enrichment).

In another example, a linear polyribonucleotide may be cyclized or concatenated by using at least one non-nucleic acid moiety. For example, the at least one non-nucleic acid moiety may react with regions or features near the 5′ terminus or near the 3′ terminus of the linear polyribonucleotide in order to cyclize or concatenate the linear polyribonucleotide. In another example, the at least one non-nucleic acid moiety may be located in or linked to or near the 5′ terminus or the 3′ terminus of the linear polyribonucleotide. The non-nucleic acid moieties may be homologous or heterologous. As a non-limiting example, the non-nucleic acid moiety may be a linkage such as a hydrophobic linkage, ionic linkage, a biodegradable linkage, or a cleavable linkage. As another non-limiting example, the non-nucleic acid moiety is a ligation moiety. As yet another non-limiting example, the non-nucleic acid moiety may be an oligonucleotide or a peptide moiety, such as an aptamer or a non-nucleic acid linker as described herein.

In another example, linear polyribonucleotides may be cyclized or concatenated by self-splicing. In some embodiments, the linear polyribonucleotides may include loop E sequence to self-ligate. In another embodiment, the linear polyribonucleotides may include a self-circularizing intron, e.g., a 5′ and 3′ slice junction, or a self-circularizing catalytic intron such as a Group I, Group II, or Group III Introns. Nonlimiting examples of group I intron self-splicing sequences may include self-splicing permuted intron-exon sequences derived from T4 bacteriophage gene td, and the intervening sequence (IVS) rRNA of Tetrahymena, cyanobacterium Anabaena pre-tRNA-Leu gene, or a Tetrahymena pre-rRNA.

In some embodiments, the polyribonucleotide may include catalytic intron fragments, such as a 3′ half of Group I catalytic intron fragment and a 5′ half of Group I catalytic intron fragment. The first and second annealing regions may be positioned within the catalytic intron fragments. Group I catalytic introns are self-splicing ribozymes that catalyze their own excision from mRNA, tRNA, and rRNA precursors via two-metal ion phorphoryl transfer mechanism. Importantly, the RNA itself self-catalyzes the intron removal without the requirement of an exogenous enzyme, such as a ligase.

In some embodiments, the 3′ half of Group I catalytic intron fragment and the 5′ half of Group I catalytic intron fragment are from a cyanobacterium Anabaena pre-tRNA-Leu gene, or a Tetrahymena pre-rRNA.

In some embodiments, the 3′ half of Group I catalytic intron fragment and the 5′ half of Group I catalytic intron fragment are from a Cyanobacterium Anabaena pre-tRNA-Leu gene, and the 3′ exon fragment includes the first annealing region and the 5′ exon fragment includes the second annealing region. The first annealing region may include, e.g., from 5 to 50, e.g., from 10 to 15 (e.g., 10, 11, 12, 13, 14, or 15) ribonucleotides and the second annealing region may include, e.g., from 5 to 50, e.g., from 10 to 15 (e.g., 10, 11, 12, 13, 14, or 15) ribonucleotides.

In some embodiments, the 3′ half of Group I catalytic intron fragment and the 5′ half of Group I catalytic intron fragment are from a Tetrahymena pre-rRNA, and the 3′ half of Group I catalytic intron fragment includes the first annealing region and the 5′ exon fragment includes the second annealing region. In some embodiments, the 3′ exon includes the first annealing region and the 5′ half of Group I catalytic intron fragment includes the second annealing region. The first annealing region may include, e.g., from 6 to 50, e.g., from 10 to 16 (e.g., 10, 11, 12, 13, 14, 15, or 16) ribonucleotides, and the second annealing region may include, e.g., from 6 to 50, e.g., from 10 to 16 (e.g., 10, 11, 12, 13, 14, 15, or 16) ribonucleotides.

In some embodiments, the 3′ half of Group I catalytic intron fragment and the 5′ half of Group I catalytic intron fragment are from a cyanobacterium Anabaena pre-tRNA-Leu gene, a Tetrahymena pre-rRNA, or a T4 phage td gene.

In some embodiments, the 3′ half of Group I catalytic intron fragment and the 5′ Group I catalytic intron fragment are from a T4 phage td gene. The 3′ exon fragment may include the first annealing region and the 5′ half of Group I catalytic intron fragment may include the second annealing region. The first annealing region may include, e.g., from 2 to 16, e.g., 10 to 16 (e.g., 2, 3, 4, 5, 6, 7, 8, 9, 10, 11, 12, 13, 14, 15, or 16) ribonucleotides, and the second annealing region may include, e.g., from 2 to 16, e.g., 10 to 16 (e.g., 2, 3, 4, 5, 6, 7, 8, 9, 10, 11, 12, 13, 14, 15, or 16) ribonucleotides.

In some embodiments, the 3′ half of Group I catalytic intron fragment is the 5′ terminus of the linear polynucleotide.

In some embodiments, the 5′ half of Group I catalytic intron fragment is the 3′ terminus of the linear polyribonucleotide.

In some embodiments, the 3′ half of Group I catalytic intron fragment has at least 80% (e.g., at least 85%, 90%, 95%, 97%, 99%, or 100%) sequence identity to the sequence of 5′-AACAACAGATAACTTACAGCTAGTCGGAAGGTGCAGAGACTCGACGGGAGCTACCCTAACGTCAAG ACGAGGGTAAAGAGAGAGTCCAATTCTCAAAGCCAATAGGCAGTAGCGAAAGCTGCGGGAGAATG-3′ (SEQ ID NO: 307).

In some embodiments, the 5′ half of Group I catalytic intron fragment has at least 80% (e.g., at least 85%, 90%, 95%, 97%, 99%, or 100%) sequence identity to the sequence of 5′-AAATAATTGAGCCTTAGAGAAGAAATTCTTTAAGTGGATGCTCTCAAACTCAGGGAAACCTAAATCTA GCTATAGACAAGGCAATCCTGAGCCAAGCCGAAGTAGTAATTAGTAAGTT-3′ (SEQ ID NO: 308).

In some embodiments, the 3′ half of Group I catalytic intron fragment has the sequence of SEQ ID NO: 307 and the 5′ half of Group I catalytic intron fragment has the sequence of SEQ ID NO: 308.

In some embodiments, the 3′ half of Group I catalytic intron fragment has at least 80% (e.g., at least 85%, 90%, 95%, 97%, 99%, or 100%) sequence identity to the sequence of 5′-CTTCTGTTGATATGGATGCAGTTCACAGACTAAATGTCGGTCGGGGAAGATGTATTCTTCTCATAAGA TATAGTCGGACCTCTCCTTAATGGGAGCTAGCGGATGAAGTGATGCAACACTGGAGCCGCTGGGAA CTAATTTGTATGCGAAAGTATATTGATTAGTTTTGGAGTACTCG-3′ (SEQ ID NO: 309).

In some embodiments, the 5′ half of Group I catalytic intron fragment has at least 80% (e.g., at least 85%, 90%, 95%, 97%, 99%, or 100%) sequence identity to the sequence of 5′-AAATAGCAATATTTACCTTTGGAGGGAAAAGTTATCAGGCATGCACCTGGTAGCTAGTCTTTAAACCA ATAGATTGCATCGGTTTAAAAGGCAAGACCGTCAAATTGCGGGAAAGGGGTCAACAGCCGTTCAGTA CCAAGTCTCAGGGGAAACTTTGAGATGGCCTTGCAAAGGGTATGGTAATAAGCTGACGGACATGGT CCTAACCACGCAGCCAAGTCCTAAGTCAACAGAT-3′ (SEQ ID NO: 310).

In some embodiments, the 3′ half of Group I catalytic intron fragment has the sequence of SEQ ID NO: 309 and the 5′ half of Group I catalytic intron fragment has the sequence of SEQ ID NO: 310.

In some embodiments, the 3′ half of Group I catalytic intron fragment has at least 80% (e.g., at least 85%, 90%, 95%, 97%, 99%, or 100%) sequence identity to the sequence of 5′-GGTTCTACATAAATGCCTAACGACTATCCCTTTGGGGAGTAGGGTCAAGTGACTCGAAACGATAGAC AACTTGCTTTAACAAGTTGGAGATATAGTCTGCTCTGCATGGTGACATGCAGCTGGATATAATTCCGG GGTAAGATTAACGACCTTATCTGAACATAATG-3′ (SEQ ID NO: 311).

In some embodiments, the 5′ half of Group I catalytic intron fragment has at least 80% (e.g., at least 85%, 90%, 95%, 97%, 99%, or 100%) sequence identity to the sequence of 5′-TAATTGAGGCCTGAGTATAAGGTGACTTATACTTGTAATCTATCTAAACGGGGAACCTCTCTAGTAGA CAATCCCGTGCTAAATTGTAGGACT-3′ (SEQ ID NO: 312).

In some embodiments, the 3′ half of Group I catalytic intron fragment has the sequence of SEQ ID NO: 311 and the 5′ half of Group I catalytic intron fragment has the sequence of SEQ ID NO: 312.

In some embodiments, the 3′ half of Group I catalytic intron fragment has at least 80% (e.g., at least 85%, 90%, 95%, 97%, 99%, or 100%) sequence identity to the sequence of 5′-TAAACAACTAACAGCTTTAGAAGGTGCAGAGACTAGACGGGAGCTACCCTAACGGATTCAGCCGAG GGTAAAGGGATAGTCCAATTCTCAACATCGCGATTGTTGATGGCAGCGAAAGTTGCAGAGAGAATGA AAATCCGCTGACTGTAAAGGTCGTGAGGGTTCGAGTCCCTCCGCCCCCA-3′ (SEQ ID NO: 313).

In some embodiments, the 5′ half of Group I catalytic intron fragment has at least 80% (e.g., at least 85%, 90%, 95%, 97%, 99%, or 100%) sequence identity to the sequence of 5′-ACGGTAGACGCAGCGGACTTAGAAAACTGGGCCTCGATCGCGAAAGGGATCGAGTGGCAGCTCTCA AACTCAGGGAAACCTAAAACTTTAAACATTMAAGTCATGGCAATCCTGAGCCAAGCTAAAGC-3′ (SEQ ID NO: 314).

In some embodiments, the 3′ half of Group I catalytic intron fragment has the sequence of SEQ ID NO: 313 and the 5′ half of Group I catalytic intron fragment has the sequence of SEQ ID NO: 314.

In some embodiments, the 3′ half of Group I catalytic intron fragment has at least 80% (e.g., at least 85%, 90%, 95%, 97%, 99%, or 100%) sequence identity to the sequence of 5′-TTAAACTCAAAATTTAAAATCCCAAATTCAAAATTCCGGGAAGGTGCAGAGACTCGACGGGAGCTAC CCTAACGTAAAGCCGAGGGTAAAGGGAGAGTCCAATTCTCAAAGCCTGAAGTTGCTGAAGCAACAA GGCAGTAGTGAAAGCTGCGAGAGAATGAAAATCCGTTGACTGTAAAAAGTCGTGGGGGTTCAAGTC CCCCCACCCCC-3′ (SEQ ID NO: 315).

In some embodiments, the 5′ half of Group I catalytic intron fragment has at least 80% (e.g., at least 85%, 90%, 95%, 97%, 99%, or 100%) sequence identity to the sequence of 5′-ATGGTAGACGCTACGGACTTAGAAAACTGAGCCTTGATAGAGAAATCTTTTAAGTGGAAGCTCTCAAA TTCAGGGAAACCTAAATCTGAATACAGATATGGCAATCCTGAGCCAAGCCCAGAAAATTTAGACTTGA GATTTGATTTTGGAG-3′ (SEQ ID NO: 316).

In some embodiments, the 3′ half of Group I catalytic intron fragment has the sequence of SEQ ID NO: 315 and the 5′ half of Group I catalytic intron fragment has the sequence of SEQ ID NO: 316.

In some embodiments, the 3′ half of Group I catalytic intron fragment has at least 80% (e.g., at least 85%, 90%, 95%, 97%, 99%, or 100%) sequence identity to the sequence of 5′-GGCTTTCAATTTGAAATCAGAAATTCAAAATTCAGGGAAGGTGCAGAGACTCGACGGGAGCTACCCT AACGTAAAGGCGAGGGTAAAGGGAGAGTCCAATTCTTAAAGCCTGAAGTTGTGCAAGCAACAAGGC AACAGTGAAAGCTGTGGAAGAATGAAAATCCGTTGACCTTAAACGGTCGTGGGGGTTCAAGTCCCCC CACCCCC-3′ (SEQ ID NO: 317).

In some embodiments, the 5′ half of Group I catalytic intron fragment has at least 80% (e.g., at least 85%, 90%, 95%, 97%, 99%, or 100%) sequence identity to the sequence of 5′-ATGGTAGACGCTACGGACTTAGAAAACTGAGCCTTGATAGAGAAATCTTTCAAGTGGAAGCTCTCAA ATTCAGGGAAACCTAAATCTGAATACAGATATGGCAATCCTGAGCCAAGCCCGGAAATTTTAGAATCA AGATTTTATTTT-3′ (SEQ ID NO: 318).

In some embodiments, the 3′ half of Group I catalytic intron fragment has the sequence of SEQ ID NO: 317 and the 5′ half of Group I catalytic intron fragment has the sequence of SEQ ID NO: 318.

In some embodiments, the 3′ half of Group I catalytic intron fragment has at least 80% (e.g., at least 85%, 90%, 95%, 97%, 99%, or 100%) sequence identity to the sequence of 5′-AGAAATGGAGAAGGTGTAGAGACTGGAAGGCAGGCACCCTAACGTTAAAGGCGAGGGTGAAGGGA CAGTCCAGACCACAAACCAGTAAATCTGGGCAGCGAAAGCTGTAGATGGTAAGCATAACCCGAAGG TCAGTGGTTCAAATCCACTTCCCGCCACCAAATTAAAAAAACAATAA-3′ (SEQ ID NO: 319).

In some embodiments, the 5′ half of Group I catalytic intron fragment has at least 80% (e.g., at least 85%, 90%, 95%, 97%, 99%, or 100%) sequence identity to the sequence of 5′-AGAAATGGAGAAGGTGTAGAGACTGGAAGGCAGGCACCCTAACGTTAAAGGCGAGGGTGAAGGGA CAGTCCAGACCACAAACCAGTAAATCTGGGCAGCGAAAGCTGTAGATGGTAAGCATAACCCGAAGG TCAGTGGTTCAAATCCACTTCCCGCCACCAAATTAAAAAAACAATAA-3′ (SEQ ID NO: 320).

In some embodiments, the 3′ half of Group I catalytic intron fragment has the sequence of SEQ ID NO: 319 and the 5′ half of Group I catalytic intron fragment has the sequence of SEQ ID NO: 320.

In some embodiments, the 3′ half of Group I catalytic intron fragment has at least 80% (e.g., at least 85%, 90%, 95%, 97%, 99%, or 100%) sequence identity to the sequence of 5′-ACAACAGATAACTTACTAACTTACAGCTAGTCGGAAGGTGCAGAGACTCGACGGGAGCTACCCTAAC GTCAAGACGAGGGTAAAGAGAGAGTCCAATTCTCAAAGCCAATAGGCAGTAGCGAAAGCTGCGGGA GAATGAAAATCCGTAGCGTCTAAACGGTCGTGTGGGTTCAAGTCCCTCCACCCCCA-3′ (SEQ ID NO: 321).

In some embodiments, the 5′ half of Group I catalytic intron fragment has at least 80% (e.g., at least 85%, 90%, 95%, 97%, 99%, or 100%) sequence identity to the sequence of 5′-AGACGCTACGGACTTAAATAATTGAGCCTTAGAGAAGAAATTCTTTAAGTGGATGCTCTCAAACTCAG GGAAACCTAAATCTAGCTATAGACAAGGCAATCCTGAGCCAAGCCGAAGTAGTAATTAGTAAGTTAG TAAGTT-3′ (SEQ ID NO: 322).

In some embodiments, the 3′ half of Group I catalytic intron fragment has the sequence of SEQ ID NO: 321 and the 5′ half of Group I catalytic intron fragment has the sequence of SEQ ID NO: 322.

In some embodiments, the 3′ half of Group I catalytic intron fragment has at least 80% (e.g., at least 85%, 90%, 95%, 97%, 99%, or 100%) sequence identity to the sequence of 5′-AACAACAGATAACTTACTAGTTACTAGTCGGAAGGTGCAGAGACTCGACGGGAGCTACCCTAACGTC AAGACGAGGGTAAAGAGAGAGTCCAATTCTCAAAGCCAATAGGCAGTAGCGAAAGCTGCGGGAGAA TGAAAATCCGTAGCGTCTAAACGGTCGTGTGGGTTCAAGTCCCTCCACCCCCA-3′ (SEQ ID NO: 323).

In some embodiments, the 5′ half of Group I catalytic intron fragment has at least 80% (e.g., at least 85%, 90%, 95%, 97%, 99%, or 100%) sequence identity to the sequence of 5′-AGACGCTACGGACTTAAATAATTGAGCCTTAGAGAAGAAATTCTTTAAGTGGATGCTCTCAAACTCAG GGAAACCTAAATCTAGCTATAGACAAGGCAATCCTGAGCCAAGCCGAAGTAGTAATTAGTAAGTT-3′ (SEQ ID NO: 324).

In some embodiments, the 3′ half of Group I catalytic intron fragment has the sequence of SEQ ID NO: 323 and the 5′ half of Group I catalytic intron fragment has the sequence of SEQ ID NO: 324.

In another example, a linear polyribonucleotide may be cyclized or concatenated by a non-nucleic acid moiety that causes an attraction between atoms, molecular surfaces at, near, or linked to the 5′ and 3′ ends of the linear polyribonucleotide. The one or more linear polyribonucleotides may be cyclized or concatenated by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In another example, the linear polyribonucleotide may comprise a ribozyme RNA sequence near the 5′ terminus and near the 3′ terminus. The ribozyme RNA sequence may covalently link to a peptide when the sequence is exposed to the remainder of the ribozyme. The peptides covalently linked to the ribozyme RNA sequence near the 5′ terminus and the 3′terminus may associate with each other, thereby causing a linear polyribonucleotide to cyclize or concatenate. In another example, the peptides covalently linked to the ribozyme RNA near the 5′ terminus and the 3′ terminus may cause the linear primary construct or linear mRNA to cyclize or concatenate after being subjected to ligated using various methods known in the art such as, but not limited to, protein ligation. Non-limiting examples of ribozymes for use in the linear primary constructs or linear polyribonucleotides of the present invention or a non-exhaustive listing of methods to incorporate or covalently link peptides are described in US patent application No. US20030082768, the contents of which is here in incorporated by reference in its entirety.

In yet another example, chemical methods of circularization may be used to generate the circular polyribonucleotide. Such methods may include but are not limited to click chemistry (e.g., alkyne and azide-based methods, or clickable bases), olefin metathesis, phosphoramidate ligation, hemiaminal-imine crosslinking, base modification, and any combination thereof.

In another example, the circular polyribonucleotide may be produced using a deoxyribonucleotide template transcribed in a cell-free system (e.g., by in vitro transcription) to a produce a linear RNA. The linear polyribonucleotide produces a splicing-compatible polyribonucleotide, which may be self-spliced to produce a circular polyribonucleotide.

In some embodiments, the disclosure provides a method of producing a circular polyribonucleotide (e.g., in a cell-free system) by providing a linear polyribonucleotide; and self-splicing linear polyribonucleotide under conditions suitable for splicing of the 3′ and 5′ splice sites of the linear polyribonucleotide; thereby producing a circular polyribonucleotide.

In some embodiments, the disclosure provides a method of producing a circular polyribonucleotide by providing a deoxyribonucleotide encoding the linear polyribonucleotide; transcribing the deoxyribonucleotide in a cell-free system to produce the linear polyribonucleotide; optionally purifying the splicing-compatible linear polyribonucleotide; and self-splicing the linear polyribonucleotide under conditions suitable for splicing of the 3′ and 5′ splice sites of the linear polyribonucleotide, thereby producing a circular polyribonucleotide.

In some embodiments, the disclosure provides a method of producing a circular polyribonucleotide by providing a deoxyribonucleotide encoding a linear polyribonucleotide; transcribing the deoxyribonucleotide in a cell-free system to produce the linear polyribonucleotide, wherein the transcribing occurs in a solution under conditions suitable for splicing of the 3′ and 5′ splice sites of the linear polyribonucleotide, thereby producing a circular polyribonucleotide. In some embodiments, the linear polyribonucleotide comprises a 5′ split-intron and a 3′ split-intron (e.g., a self-splicing construct for producing a circular polyribonucleotide). In some embodiments, the linear polyribonucleotide comprises a 5′ annealing region and a 3′ annealing region.

Suitable conditions for in vitro transcriptions and or self-splicing may include any conditions (e.g., a solution or a buffer, such as an aqueous buffer or solution) that mimic physiological conditions in one or more respects. In some embodiments, suitable conditions include between 0.1-100 mM Mg2+ ions or a salt thereof (e.g., 1-100 mM, 1-50 mM, 1-20 mM, 5-50 mM, 5-20 mM, or 5-15 mM). In some embodiments, suitable conditions include between 1-1000 mM K+ ions or a salt thereof such as KCl (e.g., 1-1000 mM, 1-500 mM, 1-200 mM, 50-500 mM, 100-500 mM, or 100-300 mM). In some embodiments, suitable conditions include between 1-1000 mM Cl− ions or a salt thereof such as KCl (e.g., 1-1000 mM, 1-500 mM, 1-200 mM, 50-500 mM, 100-500 mM, or 100-300 mM). In some embodiments, suitable conditions include between 0.1-100 mM Mn2+ ions or a salt thereof such as MnCl2 (e.g., 0.1-100 mM, 0.1-50 mM, 0.1-20 mM, 0.1-10 mM, 0.1-5 mM, 0.1-2 mM, 0.5-50 mM, 0.5-20 mM, 0.5-15 mM, 0.5-5 mM, 0.5-2 mM, or 0.1-10 mM). In some embodiments, suitable conditions include dithiothreitol (DTT) (e.g., 1-1000 μM, 1-500 μM, 1-200 μM, 50-500 μM, 100-500 μM, 100-300 μM, 0.1-100 mM, 0.1-50 mM, 0.1-20 mM, 0.1-10 mM, 0.1-5 mM, 0.1-2 mM, 0.5-50 mM, 0.5-20 mM, 0.5-15 mM, 0.5-5 mM, 0.5-2 mM, or 0.1-10 mM). In some embodiments, suitable conditions include between 0.1 mM and 100 mM ribonucleoside triphosphate (NTP) (e.g., 0.1-100 mM, 0.1-50 mM, 0.1-10 mM, 1-100 mM, 1-50 mM, or 1-10 mM). In some embodiments, suitable conditions include a pH of 4 to 10 (e.g., pH of 5 to 9, pH of 6 to 9, or pH of 6.5 to 8.5). In some embodiments, suitable conditions include a temperature of 4° C. to 50° C. (e.g., 10° C. to 40° C., 15° C. to 40° C., 20° C. to 40° C., or 30° C. to 40° C.),

In some embodiments the linear polyribonucleotide is produced from a deoxyribonucleic acid, e.g., a deoxyribonucleic acid described herein, such as a DNA vector, a linearized DNA vector, or a cDNA. In some embodiments, the linear polyribonucleotide is transcribed from the deoxyribonucleic acid by transcription in a cell-free system (e.g., in vitro transcription).

In another example, the circular polyribonucleotide may be produced in a cell, e.g., a prokaryotic cell or a eukaryotic cell. In some embodiments, an exogenous polyribonucleotide is provided to a cell (e.g., a linear polyribonucleotide described herein or a DNA molecule encoding for the transcription of a linear polyribonucleotide described here). The linear polyribonucleotides may be transcribed in the cell from an exogenous DNA molecule provided to the cell. The linear polyribonucleotide may be transcribed in the cell from an exogenous recombinant DNA molecule transiently provided to the cell. In some embodiments, the exogenous DNA molecule does not integrate into the cell's genome. In some embodiments, the linear polyribonucleotide is transcribed in the cell from a recombinant DNA molecule that is incorporated into the cell's genome.

In some embodiments, the cell is a prokaryotic cell. In some embodiments, the prokaryotic cell including the polyribonucleotides described herein may be a bacterial cell or an archaeal cell. For example, the prokaryotic cell including the polyribonucleotides described herein may be E coli, halophilic archaea (e.g., Haloferax volcaniii), Sphingomonas, cyanobacteria (e.g., Synechococcus elongatus, Spirulina (Arthrospira) spp., and Synechocystis spp.), Streptomyces, actinomycetes (e.g., Nonomuraea, Kitasatospora, or Thermobifida), Bacillus spp. (e.g., Bacillus subtilis, Bacillus anthracis, Bacillus cereus), betaproteobacteria (e.g., Burkholderia), alphaproteobacterial (e.g., Agrobacterium), Pseudomonas (e.g., Pseudomonas putida), and enterobacteria. The prokaryotic cells may be grown in a culture medium. The prokaryotic cells may be contained in a bioreactor.

The cell may be a eukaryotic cell. In some embodiments, the eukaryotic cell is a unicellular eukaryotic cell. In some embodiments, the unicellular eukaryotic is a unicellular fungal cell such as a yeast cell (e.g., Saccharomyces cerevisiae and other Saccharomyces spp., Brettanomyces spp., Schizosaccharomyces spp., Torulaspora spp, and Pichia spp.). In some embodiments, the unicellular eukaryotic cell is a unicellular animal cell. A unicellular animal cell may be a cell isolated from a multicellular animal and grown in culture, or the daughter cells thereof. In some embodiments, the unicellular animal cell may be dedifferentiated. In some embodiments, the unicellular eukaryotic cell is a unicellular plant cell. A unicellular plant cell may be a cell isolated from a multicellular plant and grown in culture, or the daughter cells thereof. In some embodiments, the unicellular plant cell may be dedifferentiated. In some embodiments, the unicellular plant cell is from a plant callus. In embodiments, the unicellular cell is a plant cell protoplast. In some embodiments, the unicellular eukaryotic cell is a unicellular eukaryotic algal cell, such as a unicellular green alga, a diatom, a euglenid, or a dinoflagellate. Non-limiting examples of unicellular eukaryotic algae of interest include Dunaliella salina, Chlorella vulgaris, Chlorella zofingiensis, Haematococcus pluvialis, Neochloris oleoabundans and other Neochloris spp., Protosiphon botryoides, Botryococcus braunii, Cryptococcus spp., Chlamydomonas reinhardtii and other Chlamydomonas spp. In some embodiments, the unicellular eukaryotic cell is a protist cell. In some embodiments, the unicellular eukaryotic cell is a protozoan cell.

In some embodiments, the eukaryotic cell is a cell of a multicellular eukaryote. For example, the multicellular eukaryote may be selected from the group consisting of a vertebrate animal, an invertebrate animal, a multicellular fungus, a multicellular alga, and a multicellular plant. In some embodiments, the eukaryotic organism is a human. In some embodiments, the eukaryotic organism is a non-human vertebrate animal. In some embodiments, the eukaryotic organism is an invertebrate animal. In some embodiments, the eukaryotic organism is a multicellular fungus. In some embodiments, the eukaryotic organism is a multicellular plant. In embodiments, the eukaryotic cell is a cell of a human or a cell of a non-human mammal such as a non-human primate (e.g., monkeys, apes), ungulate (e.g., bovids including cattle, buffalo, bison, sheep, goat, and musk ox; pig; camelids including camel, llama, and alpaca; deer, antelope; and equids including horse and donkey), carnivore (e.g., dog, cat), rodent (e.g., rat, mouse, guinea pig, hamster, squirrel), or lagomorph (e.g., rabbit, hare). In embodiments, the eukaryotic cell is a cell of a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the eukaryotic cell is a cell of an arthropod (e.g., insects, arachnids, crustaceans), a nematode, an annelid, a helminth, or a mollusc. In embodiments, the eukaryotic cell is a cell of a multicellular plant, such as an angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the eukaryotic cell is a cell of a eukaryotic multicellular alga.

The eukaryotic cells may be grown in a culture medium. The eukaryotic cells may be contained in a bioreactor.

Examples of bioreactors include, without limitation, stirred tank (e.g., well mixed) bioreactors and tubular (e.g., plug flow) bioreactors, airlift bioreactors, membrane stirred tanks, spin filter stirred tanks, vibromixers, fluidized bed reactors, and membrane bioreactors. The mode of operating the bioreactor may be a batch or continuous processes. A bioreactor is continuous when the reagent and product streams are continuously being fed and withdrawn from the system. A batch bioreactor may have a continuous recirculating flow, but no continuous feeding of reagents or product harvest. Some methods of the present disclosure are directed to large-scale production of circular polyribonucleotides. For large-scale production methods, the method may be performed in a volume of 1 liter (L) to 50 L, or more (e.g., 5 L, 10 L, 15 L, 20 L, 25 L, 30 L, 35 L, 40 L, 45 L, 50 L, or more). In some embodiments, the method may be performed in a volume of 5 L to 10 L, 5 L to 15 L, 5 L to 20 L, 5 L to 25 L, 5 L to 30 L, 5 L to 35 L, 5 L to 40 L, 5 L to 45 L, 10 L to 15 L, 10 L to 20 L, 10 L to 25 L, 20 L to 30 L, 10 L to 35 L, 10 L to 40 L, 10 L to 45 L, 10 L to 50 L, 15 L to 20 L, 15 L to 25 L, 15 L to 30 L, 15 L to 35 L, 15 L to 40 L, 15 L to 45 L, or 15 to 50 L. In some embodiments, a bioreactor may produce at least 1 g of circular RNA. In some embodiments, a bioreactor may produce 1-200 g of circular RNA (e.g., 1-10 g, 1-20 g, 1-50 g, 10-50 g, 10-100 g, 50-100 g, of 50-200 g of circular RNA). In some embodiments, the amount produced is measured per liter (e.g., 1-200 g per liter), per batch or reaction (e.g., 1-200 g per batch or reaction), or per unit time (e.g., 1-200 g per hour or per day). In some embodiments, more than one bioreactor may be utilized in series to increase the production capacity (e.g., one, two, three, four, five, six, seven, eight, or nine bioreactors may be used in series).

Methods of making the circular polyribonucleotides described herein are described in, for example, Khudyakov & Fields, Artificial DNA: Methods and Applications, CRC Press (2002); in Zhao, SYNTHETIC BIOLOGY: TOOLS AND APPLICATIONS, (First Edition), Academic Press (2013); and Egli & Herdewijn, CHEMISTRY AND BIOLOGY OF ARTIFICIAL NUCLEIC ACIDS, (First Edition), Wiley-VCH (2012).

Various methods of synthesizing circular polyribonucleotides are also described elsewhere (see, e.g., U.S. Pat. Nos. 6,210,931, 5,773,244, 5,766,903, 5,712,128, 5,426,180, US Publication No. US20100137407, International Publication No. WO1992001813, International Publication No. WO2010084371, and Petkovic et al., Nucleic Acids Res. 43:2454-65 (2015); the contents of each of which are herein incorporated by reference in their entirety).

In some embodiments, the circular polyribonucleotide is purified, e.g., free ribonucleic acids, linear or nicked RNA, DNA, proteins, etc. are removed. In some embodiments, the circular polyribonucleotides may be purified by any known method commonly used in the art. Examples of nonlimiting purification methods include, column chromatography, gel excision, size exclusion, etc.

### Linear Polyribonucleotide

The linear polyribonucleotides as disclosed herein comprise one or more expression sequences encoding one or more immunogens and/or epitopes from a coronavirus. This linear polyribonucleotide expresses the sequence encoding the one or more immunogens and/or epitopes from the coronavirus in a subject. In some embodiments, linear polyribonucleotides comprising one or more coronavirus immunogens and/or epitopes are used to produce an immune response in a subject. In some embodiments, linear polyribonucleotides comprising one or more coronavirus immunogens and/or epitopes are used to produce polyclonal antibodies as described herein.

### Coronavirus Immunogens and Epitopes

The linear polyribonucleotide comprises a sequence encoding a coronavirus immunogen or epitope. The immunogens and/or epitopes disclosed herein are associated with coronaviruses. In some embodiments, the immunogens and/or epitopes are expressed by a coronavirus or derived from an immunogen and/or epitope that is expressed by a coronavirus.

In some embodiments, an immunogen and/or epitope of the disclosure is from a predicted transcript from a SARS-CoV genome. In some embodiments, an immunogen and/or epitope of the disclosure is from a protein encoded by an open reading frame from a SARS-CoV genome. Non-limiting examples of open reading frames in SARS-CoV genomes can include ORF1a, ORF1b, spike (S), ORF3a, ORF3b, envelope (E), membrane (M), ORF6, ORF7a, ORF7b, ORF8, ORF8a, ORF8b, ORF9a, ORF9b, nucleocapsid (N), and ORF10. In some embodiments, the open reading frame from the SARS-CoV genome includes SEQ ID NO: 11.

In particular embodiments, a linear polyribonucleotide comprises a SARS-CoV-2 immunogen described in TABLE 6.

TABLE 6
Descriptions of designed linear constructs.

| | ORF | | | | | | |
|---|---|---|---|---|---|---|---|
| | (SEQ | ORF | Proline | Cloning | Circularization | 5′ | 3′ |
| Construct | ID NO.) | Description | substitutions | optimization | optimization | element | element |
| p29 | 1 (13) | S protein | Yes | Yes | No | globin | globin |
| | | transmembrane | | | | | |
| | | (TM) domain | | | | | |
| | | completely | | | | | |
| | | removed, and a | | | | | |
| | | trimerization | | | | | |
| | | domain added | | | | | |
| p30 | 3 (15) | S protein | Yes | Yes | No | globin | globin |
| | | transmembrane | | | | | |
| | | (TM) domain | | | | | |
| | | fully intact | | | | | |
| p31 | 13 (12) | S protein | N/A | N/A | N/A | globin | globin |
| | | receptor binding | | | | | |
| | | domain (RBD) | | | | | |
| | | only with | | | | | |
| | | secretion signal | | | | | |
| | | translationally | | | | | |
| | | fused to the 5′ | | | | | |
| | | end | | | | | |
| p32 | 1 (13) | S protein | Yes | Yes | Yes | globin | globin |
| | | transmembrane | | | | | |
| | | (TM) domain | | | | | |
| | | completely | | | | | |
| | | removed, and a | | | | | |
| | | trimerization | | | | | |
| | | domain added | | | | | |

In TABLE 6, “proline substitutions” denotes proline substitutions that are at residues 986 and 987, as well as a “GSAS” substitution (SEQ ID NO: 336) at the furin cleavage site (residues 682-685). For cloning optimization, single base substitution was made at coordinate 2541 to destroy a Bsal site to assist in Golden Gate Cloning construction of the plasmid DNA template. For circularization optimizations, four single nucleotides—at positions 2307, 2709, 159 and 315—were substituted to destroy sites that could potentially bind circularization elements of splint nucleic acid sequences, thereby potentially inhibiting efficient ligation. All single bp substitutions were designed to be translationally silent. Further, in TABLE 6, the 5′ Element is Globin (SEQ ID NO: 32); and the 3′ Element: Globin (SEQ ID NO: 33).

In some embodiments, the linear polyribonucleotide includes an open reading frame encoding a SARS-CoV-2 immunogen having an amino acid sequence that is at least about 80% (e.g., about 81%, 82%, 83%, 84%, 85%, 86%, 87%, 88%, 89%, 90%, 91%, 92%, 93%, 94%, 95%, 96%, 97%, 98%, 99%, or 100%) identical to any one of SEQ ID NOs: 63-111 and 293-295. In some embodiments, the linear polyribonucleotide includes an open reding frame encoding a SARS-CoV-2 immunogen having an amino acid sequence that is at least about 90% (e.g., about 91%, 92%, 93%, 94%, 95%, 96%, 97%, 98%, 99%, or 100%) identical to any one of SEQ ID NOs: 63-111 and 293-295. In some embodiments, the linear polyribonucleotide includes an open reading frame encoding a SARS-CoV-2 immunogen having an amino acid sequence that is at least about 95% (e.g., about 96%, 97%, 98%, 99%, or 100%) identical to any one of SEQ ID NOs: 63-111 and 293-295. In some embodiments, the linear polyribonucleotide includes an open reading frame encoding a SARS-CoV-2 immunogen having an amino acid sequence that is any one of SEQ ID NOs: 63-111 and 293-295. In some embodiments, the SARS-CoV-2 immunogen is an immunogenic fragment including a contiguous stretch of at least 100, 150, 200, 250, 300, 350, 400, 450, 500, 550, 600, 650, 700, 750, 800, 850, 900, 950, 1000, 1100, 1200, 1300, 1400, or 1500 amino acids of any one of SEQ ID NOs: 63-111 and 293-295. In some embodiments, the SARS-CoV-2 immunogen is an immunogenic fragment including a contiguous stretch of at least 50%, 60%, 70%, 80%, 90%, or 95% of the amino acids of a sequence any one of SEQ ID NOs: 63-111 and 293-295.

In particular embodiments, the linear polyribonucleotide includes an open reading frame with a nucleic acid sequence encoding a SARS-CoV-2 immunogen, wherein the nucleic acid sequence has at least about 80% (e.g., about 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 112-174 and 292-300. In some embodiments, the linear polyribonucleotide includes an open reading frame with a nucleic acid sequence encoding a SARS-CoV-2 immunogen, wherein the nucleic acid sequence has at least about 90% (e.g., about 91%, 92%, 93%, 94%, 95%, 96%, 97%, 98%, 99%, or 100%) sequence identity to any one of SEQ ID NOs: 112-174 and 292-300. In some embodiments, the linear polyribonucleotide includes an open reading frame with a nucleic acid sequence encoding a SARS-CoV-2 immunogen, wherein the nucleic acid sequence has at least about 95% (e.g., about 96%, 97%, 98%, 99%, or 100%) sequence identity to any one of SEQ ID NOs: 112-174 and 292-300. In certain embodiments, the linear polyribonucleotide includes an open reading frame with a nucleic acid sequence encoding a SARS-CoV-2 immunogen, wherein the nucleic acid sequence is any one of SEQ ID NOs: 112-174 and 292-300. In some embodiments, the polyribonucleotide sequence encoding the SARS-CoV-2 immunogen is a fragment including a contiguous stretch of at least 300, 400, 500, 600, 700, 800, 900, 1000, 1100, 1200, 1500, 2000, 2500, 3000, 3500, 4000, or 4500 nucleotides of any one of SEQ ID NOs: 112-174 and 292-300. In some embodiments, the polyribonucleotide sequence encoding the SARS-CoV-2 immunogen is a fragment including a contiguous stretch of at least 50%, 60%, 70%, 80%, 90%, or 95% of the amino acids of any one of SEQ ID NOs: 112-174 and 292-300.

In particular embodiments, the linear polyribonucleotide includes an open reading frame with a nucleic acid sequence encoding a SARS-CoV-2 immunogen, wherein the nucleic acid sequence has at least about 80% (e.g., about 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 219-281. In some embodiments, the linear polyribonucleotide includes an open reading frame with a nucleic acid sequence encoding a SARS-CoV-2 immunogen, wherein the nucleic acid sequence has at least about 90% (e.g., about 91%, 92%, 93%, 94%, 95%, 96%, 97%, 98%, 99%, or 100%) sequence identity to any one of SEQ ID NOs: 219-281. In some embodiments, the linear polyribonucleotide includes an open reading frame with a nucleic acid sequence encoding a SARS-CoV-2 immunogen, wherein the nucleic acid sequence has at least about 95% (e.g., about 96%, 97%, 98%, 99%, or 100%) sequence identity to any one of SEQ ID NOs: 219-281. In certain embodiments, the linear polyribonucleotide includes an open reading frame with a nucleic acid sequence encoding a SARS-CoV-2 immunogen, wherein the nucleic acid sequence is any one of SEQ ID NOs: 219-281. In some embodiments, the polyribonucleotide sequence encoding the SARS-CoV-2 immunogen is a fragment including a contiguous stretch of at least 300, 400, 500, 600, 700, 800, 900, 1000, 1100, 1200, 1500, 2000, 2500, 3000, 3500, 4000, or 4500 nucleotides of any one of SEQ ID NOs: 219-281. In some embodiments, the polyribonucleotide sequence encoding the SARS-CoV-2 immunogen is a fragment including a contiguous stretch of at least 50%, 60%, 70%, 80%, 90%, or 95% of the amino acids of any one of SEQ ID NOs: 219-281.

The disclosure specifically contemplates that any of the DNA sequences described herein may be converted to the corresponding RNA sequence and included in an RNA molecule described herein.

In some embodiments, a coronavirus epitope comprises or contains at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids, or more. In some embodiments, a coronavirus epitope comprises or contains at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, or at most 30 amino acids, or less. In some embodiments, a coronavirus epitope comprises or contains 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. In some embodiments, a coronavirus epitope contains 5 amino acids. In some embodiments, a coronavirus epitope contains 6 amino acids. In some embodiments, an epitope contains 7 amino acids. In some embodiments, a coronavirus epitope contains 8 amino acids. In some embodiments, an epitope can be about 8 to about 11 amino acids. In some embodiments, an epitope can be about 9 to about 22 amino acids.

The coronavirus immunogens may comprise immunogens recognized by B cells, immunogens recognized by T cells, or a combination thereof. In some embodiments, the immunogens comprise immunogens recognized by B cells. In some embodiments, the coronavirus immunogens are immunogens recognized by B cells. In some embodiments, the coronavirus immunogens comprise immunogens recognized by T cells. In some embodiments, the immunogens are immunogens recognized by T cells.

The coronavirus epitopes comprise epitopes recognized by B cells, epitopes recognized by T cells, or a combination thereof. In some embodiments, the coronavirus epitopes comprise epitopes recognized by B cells. In some embodiments, the epitopes are epitopes recognized by B cells. In some embodiments, the coronavirus epitopes comprise epitopes recognized by T cells. In some embodiments, the coronavirus epitopes are epitopes recognized by T cells.

Techniques for identifying immunogens and epitopes in silico have been disclosed, for example, in Sanchez-Trincado, et al. (2017), Fundamentals and methods for T-and B-cell epitope prediction, JOURNAL OF IMMUNOLOGY RESEARCH; Grifoni, Alba, et al. A Sequence Homology and Bioinformatic Approach Can Predict Candidate Targets for Immune Responses to SARS-CoV-2. CELL HOST & MICROBE (2020); Russi et al., In silico prediction of T-and B-cell epitopes in PmpD: First step towards to the design of a Chlamydia trachomatis vaccine. BIOMEDICAL JOURNAL 41.2 (2018): 109-17; Baruah, et al., Immunoinformatics-aided identification of T cell and B cell epitopes in the surface glycoprotein of 2019-nCoV, JOURNAL OF MEDICAL VIROLOGY (2020); each of which is incorporated herein by reference in its entirety.

A linear polyribonucleotide of the disclosure may comprise sequences of any number of coronavirus immunogens and/or epitopes. A linear polyribonucleotide comprises a sequence, for example, of at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more coronavirus immunogens or epitopes. In some embodiments, a linear polyribonucleotide comprises a sequence, for example, of at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more immunogens or epitopes derived from a target other than a coronavirus.

In some embodiments, a linear polyribonucleotide comprises a sequence for example, of at most 1, at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, at most 500, or less coronavirus immunogens or epitopes. In some embodiments, a linear polyribonucleotide comprises a sequence for example, of at most 1, at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, at most 500, or less immunogens or epitopes derived from a target other than a coronavirus

In some embodiments, a linear polyribonucleotide comprises a sequence, for example, of about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 coronavirus immunogens or epitopes. In some embodiments, a linear polyribonucleotide comprises a sequence, for example, of about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 immunogens or epitopes derived from a source other than a coronavirus.

A linear polyribonucleotide may comprise a sequence for one or more coronavirus epitopes from a coronavirus immunogen. For example, a coronavirus immunogen can comprise an amino acid sequence, which can contain multiple coronavirus epitopes (e.g., epitopes recognized by a B cell and/or a T cell) therein, and a linear polyribonucleotide can comprise or encode one or more of those coronavirus epitopes.

A linear polyribonucleotide comprises a sequence, for example, of at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more epitopes from one coronavirus immunogen.

In some embodiments, a linear polyribonucleotide comprises, for example, a sequence of at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, or at most 500, or less coronavirus epitopes from one coronavirus immunogen.

In some embodiments, a linear polyribonucleotide comprises a sequence, for example, of about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 coronavirus epitopes from one coronavirus immunogen.

A linear polyribonucleotide may encode variants of a coronavirus immunogen or epitope. Variants may be naturally occurring variants (for example, variants identified in sequence data from different coronavirus genera, species, isolates, or quasi-species), or may be derivative sequences as disclosed herein that have been generated in silico (for example, immunogen or epitopes with one or more amino acid insertions, deletions, substitutions, or a combination thereof compared to a wild type immunogen or epitope).

A linear polyribonucleotide comprises a sequence, for example, of at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more variants of a coronavirus immunogen or epitope.

In some embodiments, a linear polyribonucleotide comprises a sequence, for example, of at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, at most 500, or less variants of a coronavirus immunogen or epitope.

In some embodiments, a linear polyribonucleotide comprises a sequence, for example, of about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 variants of a coronavirus immunogen or epitope.

A coronavirus immunogen and/or epitope sequence of a linear polyribonucleotide can also be referred to as a coronavirus expression sequence. In some embodiments, the linear polyribonucleotide comprises one or more coronavirus expression sequences, each of which may encode a coronavirus polypeptide. The coronavirus polypeptide may be produced in substantial amounts. A coronavirus polypeptide can be a coronavirus polypeptide that is secreted from a cell, or localized to the cytoplasm, nucleus or membrane compartment of a cell. Some coronavirus polypeptides include, but are not limited to, an immunogen as disclosed herein, an epitope as disclosed herein, at least a portion of a coronavirus protein (for example, a viral envelope protein, viral matrix protein, viral spike protein, viral membrane protein, viral nucleocapsid protein, viral accessory protein, a fragment thereof, or a combination thereof). In some embodiments, a coronavirus polypeptide encoded by a linear polyribonucleotide of the disclosure comprises a fragment of a coronavirus immunogen disclosed herein. In some embodiments, a coronavirus polypeptide encoded by a linear polyribonucleotide of the disclosure comprises a fusion protein comprising two or more coronavirus immunogens disclosed herein, or fragments thereof. In some embodiments, a coronavirus polypeptide encoded by a linear polyribonucleotide of the disclosure comprises a coronavirus epitope. In some embodiments, a polypeptide encoded by a linear polyribonucleotide of the disclosure comprises a fusion protein comprising two or more coronavirus epitopes disclosed herein, for example, an artificial peptide sequence comprising a plurality of predicted epitopes from one or more coronavirus of the disclosure.

In some embodiments, exemplary coronavirus proteins that are expressed from the linear polyribonucleotide disclosed herein include a secreted protein, for example, a protein (e.g., immunogen and/or epitope) that naturally includes a signal peptide, or one that does not usually encode a signal peptide but is modified to contain one.

### Linear Polyribonucleotide Elements

The linear polyribonucleotide comprises the elements as described below as well as the coronavirus immunogen or epitope as described herein.

Linear polyribonucleotides described herein are a polyribonucleotide molecule having a 5′ and 3′ end. In some embodiments, the linear RNA has a free 5′ end or 3′ end. In some embodiments, the linear RNA has a 5′ end or 3′ end that is modified or protected from degradation. In some embodiments, the linear RNA has non-covalently linked 5′ or 3′ ends. In some embodiments, the linear RNA is an mRNA.

In some embodiments, the linear polyribonucleotide is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides.

The linear polyribonucleotides of the disclosure may include any element or combination of elements described herein, e.g., any element or combination of elements described above with respect to circular polyribonucleotides. A linear polyribonucleotide may include any one or more IRES, signal sequence, regulatory element, cleavage domain, translation initiation sequence, untranslated region, termination element, or modification as described herein (e.g., with respect to circular polyribonucleotide described above). A linear polyribonucleotide may include such elements in any number or configuration described herein (e.g., with respect to circular polyribonucleotide described above).

### Methods of Producing an Immune Response

The disclosure provides immunogenic compositions comprising a circular polyribonucleotide described above. The disclosure provides immunogenic compositions comprising a linear polyribonucleotide described above. Immunogenic compositions of the invention may comprise a diluent or a carrier, adjuvant, or any combination thereof. Immunogenic compositions of the invention may also comprise one or more immunoregulatory agents, e.g., one or more adjuvants. The adjuvants may include a TH1 adjuvant and/or a TH2 adjuvant, further discussed below. In some embodiments, the immunogenic composition comprises a diluent free of any carrier and is used for naked delivery of the circular polyribonucleotide to a subject (e.g., a subject for immunization). In some embodiments, the immunogenic composition comprises a diluent free of any carrier and is used for naked delivery of the linear polyribonucleotide to a subject.

Immunogenic compositions of the invention are used to raise an immune response in a subject (e.g., a subject for immunization). The immune response may comprise an antibody response (usually including IgG) and/or a cell-mediated immune response. In some embodiments, the immunogenic compositions are used to produce polyclonal antibodies as described herein. For example, a subject is immunized with an immunogenic composition comprising a circular polyribonucleotide comprising a coronavirus immunogen and/or epitope to stimulate production of polyclonal antibodies that bind to the coronavirus immunogen and/or epitope. In another example, a subject is immunized with an immunogenic composition comprising a linear polyribonucleotide comprising a coronavirus immunogen and/or epitope to stimulate production of polyclonal antibodies that bind to the coronavirus immunogen and/or epitope. In some embodiments, the subject is a human. In some embodiments, the subject is a non-human animal. In some embodiments, the non-human animal has a humanized immune system. In some embodiments, the subject is further immunized with an adjuvant. In some embodiments the subject is further immunized with a vaccine. Optionally, after immunization with the immunogenic composition comprising the circular polyribonucleotide, the produced polyclonal antibodies are collected and purified from the subject. Optionally, after immunization with the immunogenic composition comprising the linear polyribonucleotide, the produced polyclonal antibodies are collected and purified from the subject. In some embodiments, a composition comprises plasma collected after administration of the immunogenic composition described herein.

### Immunization

In some embodiments, methods of the disclosure comprise immunizing a subject (e.g., a subject for immunization) with an immunogenic composition comprising a circular polyribonucleotide as disclosed herein. In some embodiments, a coronavirus immunogen and/or epitope is expressed from the circular polyribonucleotide. In some embodiments, immunization induces an immune response in a subject against the coronavirus immunogen and/or epitope expressed from the circular polyribonucleotide. In some embodiments, immunization induces the production of polyclonal antibodies that bind to the coronavirus immunogen and/or epitope expressed immunogenic composition. In some embodiments, the immunogenic composition comprises the circular polyribonucleotide and a diluent, carrier, first adjuvant or a combination thereof in a single composition. In some embodiments, the subject is further immunized with a second adjuvant. In some embodiments, the subject is further immunized with a vaccine.

In some embodiments, methods of the disclosure comprise immunizing a subject (e.g., a subject for immunization) with an immunogenic composition comprising a linear polyribonucleotide as disclosed herein. In some embodiments, a coronavirus immunogen and/or epitope is expressed from the linear polyribonucleotide. In some embodiments, immunization induces an immune response in a subject against the coronavirus immunogen and/or epitope expressed from the linear polyribonucleotide. In some embodiments, immunization induces the production of polyclonal antibodies that bind to the coronavirus immunogen and/or epitope expressed from the linear polyribonucleotide. In some embodiments, an immunogenic composition comprises the linear polyribonucleotide and a diluent, carrier, first adjuvant or a combination thereof in a single composition. In some embodiments, the subject is further immunized with a second adjuvant. In some embodiments, the subject is further immunized with a vaccine.

The circular polyribonucleotide as disclosed herein stimulates the production of human polyclonal antibodies by stimulating the adaptive immune response after immunization of a subject (e.g., a subject for immunization). In some embodiments, the adaptive immune response of the subject comprises a stimulation of B lymphocytes to release polyclonal antibodies that specifically bind to the coronavirus immunogen expressed by the circular polyribonucleotide. The linear polyribonucleotide as disclosed herein stimulates the production of human polyclonal antibodies by stimulating the adaptive immune response after immunization of a subject. In some embodiments, the adaptive immune response of the subject comprises a stimulation of B lymphocytes to release polyclonal antibodies that specifically bind to the coronavirus immunogen expressed by the linear polyribonucleotide. In some embodiments, the adaptive immune response of the subject comprises stimulating cell-mediated immune responses.

The subject (e.g., a subject for immunization) is immunized with one or more immunogenic composition(s) comprising any number of circular polyribonucleotides. The subject is immunized with, for example, one or more immunogenic composition(s) comprising at least 1 circular polyribonucleotide. A non-human animal having a non-humanized immune system is immunized with, for example, one or more immunogenic composition(s) comprising at least 2, at least 3, at least 4, at least 5, at least 6, at least 7, at least 8, at least 9, at least 10, at least 11, at least 12, at least 13, at least 14, at least 15, at least 20 different circular polyribonucleotides, or more different circular polyribonucleotides. In some embodiments, a subject is immunized with one or more immunogenic composition(s) comprising at most 1 circular polyribonucleotide. In some embodiments, a subject is immunized with one or more immunogenic composition(s) comprising about 1 circular polyribonucleotide. In some embodiments, a subject is immunized with one or more immunogenic composition(s) comprising about 1-20, 1-15, 1-10, 1-9, 1-8, 1-7, 1-6, 1-5, 1-4, 1-3, 1-2, 2-20, 2-15, 2-10, 2-9, 2-8, 2-7, 2-6, 2-5, 2-4, 2-3, 3-20, 3-15, 3-10, 3-9, 3-8, 3-7, 3-6, 3-5, 3-4, 4-20, 4-15, 4-10, 4-9, 4-8, 4-7, 4-6, 4-5, 4-4, 4-3, 5-20, 5-15, 5-10, 5-9, 5-8, 5-7, 5-6, 5-10, 10-15, or 15-20 different circular polyribonucleotides. Different circular polyribonucleotides have different sequences from each other. For example, they can comprise or encode different immunogens and/or epitopes, overlapping immunogens and/or epitopes, similar immunogens and/or epitopes, or the same immunogens and/or epitopes (for example, with the same or different regulatory elements, initiation sequences, promoters, termination elements, or other elements of the disclosure). In cases where a subject is immunized with one or more immunogenic composition(s) comprising two or more different circular polyribonucleotides, the two or more different circular polyribonucleotides can be in the same or different immunogenic compositions and immunized at the same time or at different times. The immunogenic compositions comprising two or more different circular polyribonucleotides can be administered to the same anatomical location or different anatomical locations.

The two or more different circular polyribonucleotides can comprise or encode immunogens and/or epitopes from the same coronavirus, different coronavirus, or different combinations of coronaviruses disclosed herein. The two or more different circular polyribonucleotides can comprise or encode immunogens and/or epitopes from the same coronavirus or from different coronaviruses, for example, different isolates.

The subject (e.g., a subject for immunization) is immunized with one or more immunogenic composition(s) comprising any number of linear polyribonucleotides. The subject is immunized with, for example, one or more immunogenic composition(s) comprising at least 1 linear polyribonucleotide. A non-human animal having a non-humanized immune system is immunized with, for example, one or more immunogenic composition(s) comprising at least 2, at least 3, at least 4, at least 5, at least 6, at least 7, at least 8, at least 9, at least 10, at least 11, at least 12, at least 13, at least 14, at least 15, at least 20 different linear polyribonucleotides, or more different linear polyribonucleotides. In some embodiments, a subject is immunized with one or more immunogenic composition(s) comprising at most 1 linear polyribonucleotide. In some embodiments, a subject is immunized with one or more immunogenic composition(s) comprising about 1 linear polyribonucleotide. In some embodiments, a subject is immunized with one or more immunogenic composition(s) comprising about 1-20, 1-15, 1-10, 1-9, 1-8, 1-7, 1-6, 1-5, 1-4, 1-3, 1-2, 2-20, 2-15, 2-10, 2-9, 2-8, 2-7, 2-6, 2-5, 2-4, 2-3, 3-20, 3-15, 3-10, 3-9, 3-8, 3-7, 3-6, 3-5, 3-4, 4-20, 4-15, 4-10, 4-9, 4-8, 4-7, 4-6, 4-5, 4-4, 4-3, 5-20, 5-15, 5-10, 5-9, 5-8, 5-7, 5-6, 5-10, 10-15, or 15-20 different linear polyribonucleotides. Different linear polyribonucleotides have different sequences from each other. For example, they can comprise or encode different immunogens and/or epitopes, overlapping immunogens and/or epitopes, similar immunogens and/or epitopes, or the same immunogens and/or epitopes (for example, with the same or different regulatory elements, initiation sequences, promoters, termination elements, or other elements of the disclosure). In cases where a subject is immunized with one or more immunogenic composition(s) comprising two or more different linear polyribonucleotides, the two or more different linear polyribonucleotides can be in the same or different immunogenic compositions and immunized at the same time or at different times. The immunogenic compositions comprising two or more different linear polyribonucleotides can be administered to the same anatomical location or different anatomical locations.

The two or more different linear polyribonucleotides can comprise or encode immunogens and/or epitopes from the same coronavirus, different coronavirus, or different combinations of coronaviruses disclosed herein. The two or more different linear polyribonucleotides can comprise or encode immunogens and/or epitopes from the same coronavirus or from different coronaviruses, for example, different isolates.

In some embodiments, the subject (e.g., a subject for immunization) is immunized with one or more immunogenic composition(s) comprising any number of circular polyribonucleotides and one or more immunogenic composition(s) comprising any number of linear polyribonucleotides as disclosed herein. In some embodiments, an immunogenic composition disclosed herein comprises one or more circular polyribonucleotides and one or more linear polyribonucleotides as disclosed herein.

In some embodiments, an immunogenic composition comprises a circular polyribonucleotide and a diluent, a carrier, a first adjuvant, or a combination thereof. In a particular embodiment, an immunogenic composition comprises a circular polyribonucleotide described herein and a carrier or a diluent free of any carrier. In some embodiments, an immunogenic composition comprising a circular polyribonucleotide with a diluent free of any carrier is used for naked delivery of the circular polyribonucleotide to a subject. In another particular embodiment, an immunogenic composition comprises a circular polyribonucleotide described herein and a first adjuvant.

In certain embodiments, a subject (e.g., a subject for immunization) is further administered a second adjuvant. An adjuvant enhances the innate immune response, which in turn enhances the adaptive immune response for the production of polyclonal antibodies in a subject. An adjuvant can be any adjuvant as discussed below. In certain embodiments, an adjuvant is formulated with the circular polyribonucleotide as a part of an immunogenic composition. In certain embodiments, an adjuvant is not part of an immunogenic composition comprising the circular polyribonucleotide. In certain embodiments, an adjuvant is administered separately from an immunogenic composition comprising the circular polyribonucleotide. In this aspect, the adjuvant is co-administered (e.g., administered simultaneously) or administered at a different time than an immunogenic composition comprising the circular polyribonucleotide to the subject. For example, the adjuvant is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, after an immunogenic composition comprising the circular polyribonucleotide. In some embodiments, the adjuvant is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, before an immunogenic composition comprising the circular polyribonucleotide. For example, the adjuvant is administered 1, 2, 3, 4, 5, 6, 7, 14, 21, 28, 35, 42, 49, 56, 63, 70, 77, or 84 days, or any day therebetween, after an immunogenic composition comprising the circular polyribonucleotide. In some embodiments, the adjuvant is administered 1, 2, 3, 4, 5, 6, 7, 14, 21, 28, 35, 42, 49, 56, 63, 70, 77, or 84 days, or any day therebetween, before an immunogenic composition comprising the circular polyribonucleotide. The adjuvant is administered to the same anatomical location or different anatomical location as the immunogenic composition comprising the circular polyribonucleotide.

In some embodiments, an immunogenic composition comprises a linear polyribonucleotide and a diluent, a carrier, a first adjuvant, or a combination thereof. In a particular embodiment, an immunogenic composition comprises a linear polyribonucleotide described herein and a carrier or a diluent free of any carrier. In some embodiments, an immunogenic composition comprising a linear polyribonucleotide with a diluent free of any carrier is used for naked delivery of the linear polyribonucleotide to a subject (e.g., a subject for immunization). In another particular embodiment, an immunogenic composition comprises a linear polyribonucleotide described herein and a first adjuvant.

In certain embodiments, a subject (e.g., a subject for immunization) is further administered a second adjuvant. An adjuvant enhances the innate immune response, which in turn enhances the adaptive immune response for the production of polyclonal antibodies in a subject. An adjuvant can be any adjuvant as discussed below. In certain embodiments, an adjuvant is formulated with the linear polyribonucleotide as a part of an immunogenic composition. In certain embodiments, an adjuvant is not part of an immunogenic composition comprising the linear polyribonucleotide. In certain embodiments, an adjuvant is administered separately from an immunogenic composition comprising the linear polyribonucleotide. In this aspect, the adjuvant is co-administered (e.g., administered simultaneously) or administered at a different time than an immunogenic composition comprising the linear polyribonucleotide to the subject. For example, the adjuvant is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, after an immunogenic composition comprising the linear polyribonucleotide. In some embodiments, the adjuvant is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, before an immunogenic composition comprising the linear polyribonucleotide. For example, the adjuvant is administered 1, 2, 3, 4, 5, 6, 7, 14, 21, 28, 35, 42, 49, 56, 63, 70, 77, or 84 days, or any day therebetween, after an immunogenic composition comprising the linear polyribonucleotide. In some embodiments, the adjuvant is administered 1, 2, 3, 4, 5, 6, 7, 14, 21, 28, 35, 42, 49, 56, 63, 70, 77, or 84 days, or any day therebetween, before an immunogenic composition comprising the linear polyribonucleotide. The adjuvant is administered to the same anatomical location or different anatomical location as the immunogenic composition comprising the linear polyribonucleotide.

In some embodiments, a subject (e.g., a subject for immunization) is further immunized with a second agent, e.g., a vaccine (as described below) that is not a circular polyribonucleotide. The vaccine is co-administered (e.g., administered simultaneously) or administered at a different time than an immunogenic composition comprising the circular polyribonucleotide to the subject. For example, the vaccine is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, after an immunogenic composition comprising the circular polyribonucleotide. In some embodiments, the vaccine is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, before an immunogenic composition comprising the circular polyribonucleotide. For example, the vaccine is administered 1, 2, 3, 4, 5, 6, 7, 14, 21, 28, 35, 42, 49, 56, 63, 70, 77, or 84 days, or any day therebetween, after an immunogenic composition comprising the circular polyribonucleotide. In some embodiments, the vaccine is administered 1, 2, 3, 4, 5, 6, 7, 14, 21, 28, 35, 42, 49, 56, 63, 70, 77, or 84 days, or any day therebetween, before an immunogenic composition comprising the circular polyribonucleotide.

In some embodiments, a subject (e.g., a subject for immunization) is further immunized with a second agent, e.g., a vaccine (as described below) that is not a linear polyribonucleotide. The vaccine is co-administered (e.g., administered simultaneously) or administered at a different time than an immunogenic composition comprising the linear polyribonucleotide to the subject. For example, the vaccine is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, after an immunogenic composition comprising the linear polyribonucleotide. In some embodiments, the vaccine is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, before an immunogenic composition comprising the linear polyribonucleotide. For example, the vaccine is administered 1, 2, 3, 4, 5, 6, 7, 14, 21, 28, 35, 42, 49, 56, 63, 70, 77, or 84 days, or any day therebetween, after an immunogenic composition comprising the linear polyribonucleotide. In some embodiments, the vaccine is administered 1, 2, 3, 4, 5, 6, 7, 14, 21, 28, 35, 42, 49, 56, 63, 70, 77, or 84 days, or any day therebetween, before an immunogenic composition comprising the linear polyribonucleotide.

A subject (e.g., a subject for immunization) can be immunized with an immunogenic composition, adjuvant, vaccine (e.g., protein subunit vaccine), or a combination thereof any suitable number of times to achieve a desired response. For example, a prime-boost immunization strategy can be utilized to generate hyperimmune plasma containing a high concentration of antibodies that bind to immunogens and/or epitopes of the disclosure. A subject can be immunized with an immunogenic composition, adjuvant, vaccine (e.g., protein subunit vaccine), or a combination thereof, of the disclosure, for example, at least 1, at least 2, at least 3, at least 4, at least 5, at least 6, at least 7, at least 8, at least 9, at least 10, or at least 15 times, or more.

In some embodiments, a subject (e.g., a subject for immunization) can be immunized with an immunogenic composition, adjuvant, vaccine (e.g., protein subunit vaccine), or a combination thereof, of the disclosure at most 2, at most 3, at most 4, at most 5, at most 6, at most 7, at most 8, at most 9, at most 10, at most 15, or at most 20 times, or less.

In some embodiments, a subject (e.g., a subject for immunization) can be immunized with an immunogenic composition, adjuvant, vaccine (e.g., protein subunit vaccine), or a combination thereof, of the disclosure about 1, 2, 3, 4, 5, 6, 7, 8, 9, 10, 15, or 20 times.

In some embodiments, a subject (e.g., a subject for immunization) can be immunized with an immunogenic composition, adjuvant, vaccine (e.g., protein subunit vaccine), or a combination thereof, of the disclosure once. In some embodiments, a subject can be immunized with an immunogenic composition, adjuvant, vaccine (e.g., protein subunit vaccine), or a combination thereof, of the disclosure twice. In some embodiments, a subject can be immunized with an immunogenic composition, adjuvant, vaccine (e.g., protein subunit vaccine), or a combination thereof, of the disclosure three times. In some embodiments, a subject can be immunized with an immunogenic composition, adjuvant, vaccine (e.g., protein subunit vaccine), or a combination thereof, of the disclosure four times. In some embodiments, a subject can be immunized with an immunogenic composition, adjuvant, vaccine (e.g., protein subunit vaccine), or a combination thereof, of the disclosure five times. In some embodiments, a subject can be immunized with an immunogenic composition, adjuvant, vaccine (e.g., protein subunit vaccine), or a combination thereof, of the disclosure seven times.

Suitable time intervals can be selected for spacing two or more immunizations. The time intervals can apply to multiple immunizations with the same immunogenic composition, adjuvant, or vaccine (e.g., protein subunit vaccine), or combination thereof, for example, the same the same immunogenic composition, adjuvant, or vaccine (e.g., protein subunit vaccine), or combination thereof, can be administered in the same amount or a different amount, via the same immunization route or a different immunization route. The time intervals can apply to immunizations with different agents, for example, a first immunogenic composition comprising a first circular polyribonucleotide and a second immunogenic composition comprising s second circular polyribonucleotide. The time intervals can apply to a first immunogenic composition comprising a first linear polyribonucleotide and a second immunogenic composition comprising s second linear polyribonucleotide. For regimens comprising three or more immunizations, the time intervals between immunizations can be the same or different. In some examples, about 1, 2, 3, 4, 5, 6, 7, 8, 9, 10, 11, 12, 14, 16, 17, 18, 20, 22, 24, 26, 28, 30, 32, 34, 36-, 40-, 48-, or 72-hours elapse between two immunizations. In some embodiments, about 1, 2, 3, 4, 5, 6, 7, 8, 9, 10, 11, 12, 14, 16, 17, 18, 20, 21-, 24-, 28-, or 30-days elapse between two immunizations. In some embodiments, about 1, 2, 3, 4, 5-, 6-, 7-, or 8-weeks elapse between two immunizations. In some embodiments, about 1, 2, 3, 4, 5-, 6-, 7-, or 8-months elapse between two immunizations.

In some embodiments, at least 1, at least 2, at least 3, at least 4, at least 5, at least 6, at least 7, at least 8, at least 9, at least 10, at least 15, at least 20, at least 24, at least 36, or at least 72 hours, or more elapse between two immunizations. In some embodiments, at most 1, at most 2, at most 3, at most 4, at most 5, at most 6, at most 7, at most 8, at most 9, at most 10, at most 15, at most 20, at most 24, at most 36, or at most 72 hours, or less elapse between two immunizations.

In some embodiments, at least 1, at least 2, at least 3, at least 4, at least 5, at least 6, at least 7, at least 8, at least 9, at least 10, at least 15, at least 20, at least 21, at least 22, at least 23, at least 24, at least 25, at least 26 at least 27, at least 28, at least 29, or at least 30 days, or more, elapse between two immunizations. In some embodiments, at most 2, at most 3, at most 4, at most 5, at most 6, at most 7, at most 8, at most 9, at most 10, at most 15, at most 20, at most 21, at most 22, at most 23, at most 24, at most 25, at most 26, at most 27, at most 28, at most 29, at most 30, at most 32, at most 34, or at most 36 days, or less elapse between two immunizations.

In some embodiments, at least 1, at least 2, at least 3, at least 4, at least 5, at least 6, at least 7, or at least 8 weeks, or more elapse between two immunizations. In some embodiments, at most 2, at most 3, at most 4, at most 5, at most 6, at most 7, at most 8 weeks, or less elapse between two immunizations.

In some embodiments, at least 1, at least 2, at least 3, at least 4, at least 5, at least 6, at least 7, or at least 8 months, or more elapse between two immunizations. In some embodiments, at most 2, at most 3, at most 4, at most 5, at most 6, at most 7, at most 8 months, or less elapse between two immunizations.

In some embodiments, a non-human animal having a humanized immune system is immunized 3 times at 3-4-week intervals.

In some embodiments, the method further comprises pre-administering an agent to improve immunogenic responses to the non-human animal (e.g., the non-human animal having a humanized immune system) or human subject (e.g., a non-human animal or human subject for immunization). In some embodiments, the agent is the immunogen as disclosed herein (e.g., a protein immunogen). For example, the method comprises administering the protein immunogen from 1 to 7 days prior to administration of the circular polyribonucleotide comprising the sequence encoding the protein immunogen. In some embodiments, the protein immunogen is administered 1, 2, 3, 4, 5, 6, or 7 days prior to administration of the circular polyribonucleotide comprising the sequence encoding the protein immunogen. For example, the method comprises administering the protein immunogen from 1 to 7 days prior to administration of the linear polyribonucleotide comprising the sequence encoding the protein immunogen. In some embodiments, the protein immunogen is administered 1, 2, 3, 4, 5, 6, or 7 days prior to administration of the linear polyribonucleotide comprising the sequence encoding the protein immunogen. The protein immunogen may be administered as a protein preparation, encoded in a plasmid (pDNA), presented in a virus-like particle (VLP), formulated in a lipid nanoparticle, or the like.

A subject (e.g., a subject for immunization) can be immunized with an immunogenic composition, an adjuvant, or a vaccine (e.g., protein subunit vaccine), or a combination thereof, at any suitable number anatomical sites. The same immunogenic composition, an adjuvant, a vaccine (e.g., protein subunit vaccine), or a combination thereof can be administered to multiple anatomical sites, different immunogenic compositions comprising the same or different circular polyribonucleotides, adjuvants, vaccines (e.g., protein subunit vaccine) or a combination thereof can be administered to different anatomical sites, different immunogenic compositions comprising the same or different circular polyribonucleotides, adjuvants, vaccines (e.g., protein subunit vaccines) or a combination thereof can be administered to the same anatomical site, or any combination thereof. For example, an immunogenic composition comprising a circular polyribonucleotide can be administered in to two different anatomical sites, and/or an immunogenic composition comprising a circular polyribonucleotide can be administered to one anatomical site, and an adjuvant can be administered to a different anatomical site. The same immunogenic composition, an adjuvant, a vaccine (e.g., protein subunit vaccine), or a combination thereof can be administered to multiple anatomical sites, different immunogenic compositions comprising the same or different linear polyribonucleotides, adjuvants, vaccines (e.g., protein subunit vaccine) or a combination thereof can be administered to different anatomical sites, different immunogenic compositions comprising the same or different linear polyribonucleotides, adjuvants, vaccines (e.g., protein subunit vaccines) or a combination thereof can be administered to the same anatomical site, or any combination thereof. For example, an immunogenic composition comprising a linear polyribonucleotide can be administered in to two different anatomical sites, and/or an immunogenic composition comprising a linear polyribonucleotide can be administered to one anatomical site, and an adjuvant can be administered to a different anatomical site.

Immunization at any two or more anatomical routes can be via the same route of immunization (e.g., intramuscular) or by two or more routes of immunization. In some embodiments, an immunogenic composition comprising a circular polyribonucleotide, an adjuvant, or a vaccine (e.g., protein subunit vaccine), or a combination thereof, of the disclosure is immunized to at least 1, at least 2, at least 3, at least 4, at least 5, or at least 6 anatomical sites of a subject (e.g., a subject for immunization). In some embodiments, an immunogenic composition comprising a circular polyribonucleotide, an adjuvant, or a vaccine (e.g., protein subunit vaccine), or a combination thereof, of the disclosure is immunized to at most 2, at most 3, at most 4, at most 5, at most 6, at most 7, at most 8, at most 9, or at most 10 anatomical sites of the subject, or less. In some embodiments, an immunogenic composition comprising a circular polyribonucleotide, or an adjuvant of the disclosure is immunized to 1, 2, 3, 4, 5, 6, 7, 8, 9, 10 anatomical sites of a subject. In some embodiments, an immunogenic composition comprising a linear polyribonucleotide, an adjuvant, or a vaccine (e.g., protein subunit vaccine), or a combination thereof, of the disclosure is immunized to at least 1, at least 2, at least 3, at least 4, at least 5, or at least 6 anatomical sites of a subject. In some embodiments, an immunogenic composition comprising a linear polyribonucleotide, an adjuvant, or a vaccine (e.g., protein subunit vaccine), or a combination thereof, of the disclosure is immunized to at most 2, at most 3, at most 4, at most 5, at most 6, at most 7, at most 8, at most 9, or at most 10 anatomical sites of the subject, or less. In some embodiments, an immunogenic composition comprising a linear polyribonucleotide, or an adjuvant of the disclosure is immunized to 1, 2, 3, 4, 5, 6, 7, 8, 9, 10 anatomical sites of a subject.

Immunization can be by any suitable route. Non-limiting examples of immunization routes include intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intrasternal, intracerebral, intraocular, intralesional, intracerebroventricular, intracisternal, or intraparenchymal, e.g., injection and infusion. In some cases, immunization can be via inhalation. Two or more immunizations can be done by the same route or by different routes.

Any suitable amount of a circular polyribonucleotide can be administered to a subject (e.g., a subject for immunization) of the disclosure. For example, a subject can be immunized with at least about 1 ng, at least about 10 ng, at least about 100 ng, at least about 1 μg, at least about 10 μg, at least about, at least about 100 μg, at least about 1 mg, at least about 10 mg, at least about 100 mg, or at least about 1 g of a circular polyribonucleotide. In some embodiments, a subject can be immunized with at most about 1 ng, at most about 10 ng, at most about 100 ng, at most about 1 μg, at most about 10 μg, at most about, at most about 100 μg, at most about 1 mg, at most about 10 mg, at most about 100 mg, or at most about 1 g of a circular polyribonucleotide. In some embodiments, a subject can be immunized with about 1 ng, about 10 ng, about 100 ng, about 1 μg, about 10 μg, about, about 100 μg, about 1 mg, about 10 mg, about 100 mg, or about 1 g of a circular polyribonucleotide.

Any suitable amount of a linear polyribonucleotide can be administered to a subject (e.g., a subject for immunization) of the disclosure. For example, a subject can be immunized with at least about 1 ng, at least about 10 ng, at least about 100 ng, at least about 1 μg, at least about 10 μg, at least about 100 μg, at least about 1 mg, at least about 10 mg, at least about 100 mg, or at least about 1 g of a linear polyribonucleotide. In some embodiments, a subject can be immunized with at most about 1 ng, at most about 10 ng, at most about 100 ng, at most about 1 μg, at most about 10 μg, at most about, at most about 100 μg, at most about 1 mg, at most about 10 mg, at most about 100 mg, or at most about 1 g of a linear polyribonucleotide. In some embodiments, a subject can be immunized with about 1 ng, about 10 ng, about 100 ng, about 1 μg, about 10 μg, about, about 100 μg, about 1 mg, about 10 mg, about 100 mg, or about 1 g of a linear polyribonucleotide.

In some embodiments, the method further comprises evaluating the non-human animal or human subject (e.g., a subject for immunization) for antibody response to the immunogen. In some embodiments, the evaluating is before and/or after administration of the circular polyribonucleotide comprising a sequence encoding a coronavirus immunogen. In some embodiments, the evaluating is before and/or after administration of the linear polyribonucleotide comprising a sequence encoding a coronavirus immunogen.

### Adjuvants

An adjuvant enhances the immune responses (humoral and/or cellular) elicited in a subject (e.g., a subject for immunization) who receives the adjuvant and/or an immunogenic composition comprising the adjuvant. In some embodiments, an adjuvant is administered to a subject (e.g., a subject for immunization) for the production of polyclonal antibodies from a circular polyribonucleotide as disclosed herein. In some embodiments, an adjuvant is administered to a subject for the production of polyclonal antibodies from a linear polyribonucleotide as disclosed herein. In some embodiments, an adjuvant is used in the methods described herein to produce polyclonal antibodies as described herein. In a particular embodiment, an adjuvant is used to promote production of the polyclonal antibodies in a subject against a coronavirus immunogen and/or epitope expressed from a circular polyribonucleotide. In some embodiments, an adjuvant and circular polyribonucleotide are co-administered in separate compositions. In some embodiments, an adjuvant is mixed or formulated with a circular polyribonucleotide in a single composition to obtain an immunogenic composition that is administered to a subject. In a particular embodiment, an adjuvant is used to promote production of the polyclonal antibodies in a subject against a coronavirus immunogen and/or epitope expressed from a linear polyribonucleotide. In some embodiments, an adjuvant and linear polyribonucleotide are co-administered in separate compositions. In some embodiments, an adjuvant is mixed or formulated with a linear polyribonucleotide in a single composition to obtain an immunogenic composition that is administered to a subject.

An adjuvant may be a component of a polyribonucleotide. An adjuvant may be a polypeptide adjuvant encoded by an expression sequence of a polyribonucleotide, may be a molecule (e.g., a small molecule, polypeptide, or nucleic acid molecule) that is not encoded by the polyribonucleotide. An adjuvant may be formulated with a polyribonucleotide in the same pharmaceutical composition. An adjuvant may be administered separately (e.g., as a separate pharmaceutical composition) in combination with a polyribonucleotide.

In some embodiments, the adjuvant is encoded by the polyribonucleotide. In some embodiments, the polyribonucleotide encodes more than one adjuvant. For example, the polyribonucleotide encodes between 2 and 100 adjuvants. In some embodiments, the polyribonucleotide encodes between 2 and 10 adjuvants. In some embodiments, the polyribonucleotide encodes 2 adjuvants. One or more of the adjuvants encoded by a polyribonucleotide may include an N-terminal signal sequence, e.g., that directs the expressed polypeptide adjuvant to the secretory pathway. In some embodiments, the polyribonucleotide encodes 3 adjuvants. In some embodiments, the polyribonucleotide encodes 4 adjuvants. In some embodiments, the polyribonucleotide encodes 5 adjuvants. In some embodiments, the adjuvant is encoded by the same polyribonucleotide that encodes one or more immunogens. The adjuvant(s) and immunogen(s) may be co-delivered on the same polyribonucleotide. In some embodiments, the adjuvant encoded by the polyribonucleotide is a sequence (e.g., a polyribonucleotide sequence) that is an innate immune system stimulator. The innate immune system stimulator sequence may include at least 5, at least 10, at least 20, at least 50, at least 100, or at least 500 ribonucleotides. The innate immune system stimulator sequence may include between 5 and 1000, between 10 and 500, between 20 and 500, between 10 and 100, between 20 and 100, between 20 and 50, between 100 and 500, between 500 and 1000, or between 10 and 1000 ribonucleotides. For example, a sequence that is an innate immune system stimulator may be selected from a GU-rich motif, an AU-rich motif, a structured region including dsRNA, or an aptamer.

Adjuvants may be a TH1 adjuvant and/or a TH2 adjuvant. Further adjuvants contemplated by this disclosure include, but are not limited to, one or more of the following:

Mineral-containing compositions. Mineral-containing compositions suitable for use as adjuvants in the disclosure include mineral salts, such as aluminum salts, and calcium salts. The disclosure includes mineral salts such as hydroxides (e.g., oxyhydroxides), phosphates (e.g., hydroxyphosphates, orthophosphates), sulphates, etc., or mixtures of different mineral compounds, with the compounds taking any suitable form (e.g., gel, crystalline, amorphous, etc.). Calcium salts include calcium phosphate (e.g., the “CAP”). Aluminum salts include hydroxides, phosphates, sulfates, and the like.

Oil emulsion compositions. Oil-emulsion compositions suitable for use as adjuvants in the disclosure include squalene-water emulsions, such as MF59 (5% Squalene, 0.5% Tween 80 and 0.5% Span, formulated into submicron particles using a microfluidizer), AS03 (α-tocopherol, squalene and polysorbate 80 in an oil-in-water emulsion), Montanide formulations (e.g., Montanide ISA 51, Montanide ISA 720), incomplete Freunds adjuvant (IFA), complete Freund's adjuvant (CFA), and incomplete Freund's adjuvant (IFA).

Small molecules. Small molecules suitable for use as adjuvants in the disclosure include imiquimod or 847, resiquimod or R848, and gardiquimod.

Polymeric nanoparticles. Polymeric nanoparticles suitable for use as an adjuvant in the disclosure include poly(α-hydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, and combinations thereof.

Saponin (i.e., a glycoside, polycyclic aglycones attached to one or more sugar side chains). Saponin formulations suitable for use as an adjuvant in the disclosure include purified formulations, such as QS21, as well as lipid formulations, such as ISCOMs and ISCOMs matrix. QS21 is marketed as STIMULON™. Saponin formulations may also include a sterol, such as cholesterol. Combinations of saponins and cholesterols can be used to form unique particles called immune-stimulating complexes (ISCOMs). ISCOMs typically also include a phospholipid such as phosphatidylethanolamine or phosphatidylcholine. Any known saponin can be used in ISCOMs. Preferably, the ISCOM includes one or more of QuilA, QHA & QHC. Optionally, the ISCOMS may be devoid of additional detergent.

Lipopolysaccharides. Adjuvants suitable for use in the disclosure include non-toxic derivatives of enterobacterial lipopolysaccharide (LPS). Such derivatives include monophosphoryl lipid A (MPLA), glucopyranosyl lipid A (GLA) and 3-O-deacylated MPL (3dMPL). 3dMPL is a mixture of 3 De-O-acylated monophosphoryl lipid A with 4, 5 or 6 acylated chains. Other non-toxic LPS derivatives include monophosphoryl lipid A mimics, such as aminoalkyl glucosaminide phosphate derivatives e.g., RC-529.

Liposomes. Liposomes suitable for use as an adjuvant in the disclosure include virosomes and CAF01.

Lipid nanoparticles. Adjuvants suitable for use in the disclosure include lipid nanoparticles (LNPs) and their components.

Lipopeptides (i.e., compounds including one or more fatty acid residues and two or more amino acid residues). Lipopeptide suitable for use as an adjuvant in the disclosure include Pam2 (Pam2CSK4) and Pam3 (Pam3CSK4).

Glycolipids. Glycolipids suitable for use as an adjuvant in the disclosure include cord factor (trehalose dimycolate).

Peptides and peptidoglycans derived from (synthetic or purified) gram-negative or gram-positive bacteria, such as MDP (N-acetyl-muramyl-L-alanyl-D-isoglutamine) are suitable for use as an adjuvant in the disclosure Carbohydrates (carbohydrate containing) or polysaccharides suitable for use as an adjuvant include dextran (e.g., branched microbial polysaccharide), dextran-sulfate, lentinan, zymosan, beta-glucan, deltin, mannan, and chitin.

RNA based adjuvants. RNA based adjuvants suitable for use in the disclosure are poly IC, poly IC:LC, hairpin RNAs with or without a 5′triphosphate, viral sequences, polyU containing sequence, dsRNA natural or synthetic RNA sequences (e.g., poly I:C), and nucleic acid analogs (e.g., cyclic GMP-AMP or other cyclic dinucleotides e.g., cyclic di-GMP, immunostimulatory base analogs e.g., C8-substituted and N7,C8-disubstituted guanine ribonucleotides). In some embodiments, the adjuvant is the linear polyribonucleotide counterpart of the circular polyribonucleotide described herein.

DNA based adjuvants. DNA based adjuvants suitable for use in the disclosure include CpGs (e.g., CpG1018), dsDNA, and natural or synthetic immunostimulatory DNA sequences.

Proteins or peptides. Proteins and peptides suitable for use as an adjuvant in the disclosure include flagellin-fusion proteins, MBL (mannose-binding lectin), cytokines, and chemokines.

Viral particles. Viral particles suitable for use as an adjuvant include virosomes (phospholipid cell membrane bilayer).

An adjuvant for use in the disclosure may be bacterial derived, such as a flagellin, LPS, or a bacterial toxin (e.g., enterotoxins (protein), e.g., heat-labile toxin or cholera toxin). An adjuvant for use in the disclosure may be a hybrid molecule such as CpG conjugated to imiquimod. An adjuvant for use in the disclosure may be a fungal or oomycete microbe-associated molecular patterns (MAMPs), such as chitin or beta-glucan. In some embodiments, an adjuvant is an inorganic nanoparticle, such as gold nanorods or silica-based nanoparticles (e.g., mesoporous silica nanoparticles (MSN)). In some embodiments, an adjuvant is a multi-component adjuvant or adjuvant system, such as AS01 (AS01B), ASO3, ASO4 (MLP5+alum), alum (mixture of aluminum hydroxide and magnesium hydroxide), aluminum hydroxide, magnesium hydroxide, CFA (complete Freund's adjuvant: IFA+peptiglycan+trehalose dimycolate), CAF01 (two component system of cationic liposome vehicle (dimethyl dioctadecyl-ammonium (DDA)) stabilized with a glycolipid immunomodulator (trehalose 6,6-dibehenate (TDB), which can be a synthetic variant of cord factor located in the mycobacterial cell wall).

Cytokines. An adjuvant may be a partial or full-length DNA encoding a cytokine such as, a pro-inflammatory cytokine (e.g., GM-CSF, IL-1 alpha, IL-1 beta, TGF-beta, TNF-alpha, TNF-beta), Th-1 inducing cytokines (e.g., IFN-gamma, IL-2, IL-12, IL-15, IL-18), or Th-2 inducing cytokines (e.g., IL-4, IL-5, IL-6, IL-10, IL-13).

Chemokines. An adjuvant may be a partial or full-length DNA or RNA (e.g., circular polyribonucleotide or mRNA) encoding a chemokine such as, MCP-1, MIP-1 alpha, MIP-1 beta, Rantes, or TCA-3.

An adjuvant may be a partial or full-length DNA encoding a costimulatory molecule, such as CD80, CD86, CD40-L, CD70, or CD27.

An adjuvant may be a partial or full length DNA or RNA (e.g., circular polyribonucleotide or mRNA) encoding for an innate immune system stimulator (partial, full-length, or mutated) such as TLR4, TLR3, TLR3, TLR9, TLR7, TLR8, TLR7, RIG-I/DDX58, or MDA-5/IFIH1; or a constitutively active (ca) innate immune stimulator, such as caTLR4, caTLR3, caTLR3, caTLR9, caTLR7, caTLR8, caTLR7, caRIG-I/DDX58, or caMDA-5/IFIH1.

An adjuvant may be a partial or full-length DNA or RNA (e.g., circular polyribonucleotide or mRNA) encoding for an adaptor or signaling molecule, such as STING (e.g., caSTING), TRIF, TRAM, MyD88, IPS1, ASC, MAVS, MAPKs, IKK-alpha, IKK complex, TBK1, beta-catenin, and caspase 1.

An adjuvant may be a partial or full-length DNA or RNA (e.g., circular polyribonucleotide or mRNA) encoding for a transcriptional activator, such as a transcription activator that can upregulate an immune response (e.g., AP1, NF-kappa B, IRF3, IRF7, IRF1, or IRF5). An adjuvant may be a partial or full-length DNA encoding for a cytokine receptor, such as IL-2beta, IFN-gamma, or IL-6.

An adjuvant may be a partial or full-length DNA or RNA (e.g., circular polyribonucleotide or mRNA) encoding for a bacterial component, such as flagellin or MBL.

An adjuvant may be a partial or full-length DNA or RNA (e.g., circular polyribonucleotide or mRNA) encoding for any component of the innate immune system.

In some embodiments, a subject is administered a polyribonucleotide encoding one or more immunogens in combination with an adjuvant (e.g., an adjuvant that is a separate molecular entity from the polyribonucleotide or an adjuvant that is encoded on a separate polyribonucleotide). The term “in combination with” as used throughout the description includes any two compositions administered as part of a therapeutic regimen. This may include, for example, a polyribonucleotide and an adjuvant formulated as a single pharmaceutical composition. This also includes, for example, a polyribonucleotide and an adjuvant administered to a subject as separate compositions according to a defined therapeutic or dosing regimen. An adjuvant may be administered to a subject before, at substantially the same time, or after the administration of a polyribonucleotide. An adjuvant may be administered within 1 day, 2 days, 5 days, 10 days, 20 days, 1 month, 2 months, 3 months, 4 months, 5 months, or 6 months before or after administration of a polyribonucleotide. An adjuvant may be administered by the same route of administration (e.g., intradermal, intramuscularly, subcutaneously, intravenously, intraperitoneally, topically, or orally) or a different route than a polyribonucleotide.

### Vaccine

In some embodiments of methods described herein, a second agent is also administered to the subject (e.g., a subject for immunization), e.g., a second vaccine is also administered to a subject (e.g., a subject for immunization). In some embodiments, a composition that is administered to a subject comprises a polyribonucleotide described herein and a second vaccine. In some embodiments, a vaccine and polyribonucleotide are co-administered in separate compositions. The vaccine is simultaneously administered with the polyribonucleotide immunization, administered before the polyribonucleotide immunization, or after the polyribonucleotide immunization.

For example, in some embodiments, a subject (e.g., a subject for immunization) is immunized with a non-polyribonucleotide coronavirus vaccine (e.g., protein subunit vaccine) and an immunogenic composition comprising a polyribonucleotide. In some embodiments, a subject is immunized with a non-polyribonucleotide vaccine for a first microorganism (e.g., pneumococcus) and an immunogenic composition comprising a polyribonucleotide as disclosed herein. A vaccine can be any bacterial infection vaccine or viral infection vaccine. In a particular embodiment, a vaccine is a pneumococcal polysaccharide vaccine, such as PCV13 or PPSV23. In some embodiments, the vaccine is an influenza vaccine. In some embodiments, the vaccine is an RSV vaccine (e.g., palivizumap).

In some embodiments, a composition that is administered to a subject comprises a linear polyribonucleotide and a vaccine. In some embodiments, a vaccine and linear polyribonucleotide are co-administered in separate compositions. The vaccine is simultaneously administered with the linear polyribonucleotide immunization, administered before the linear polyribonucleotide immunization, or after the linear polyribonucleotide immunization.

For example, in some embodiments, a subject (e.g., a subject for immunization) is immunized with a polyribonucleotide (e.g., non-linear polyribonucleotide) coronavirus vaccine (e.g., protein subunit vaccine) and an immunogenic composition comprising a linear polyribonucleotide as disclosed herein comprising a sequence encoding a coronavirus immunogen. In some embodiments, a subject is immunized with a non-polyribonucleotide vaccine for a first microorganism (e.g., pneumococcus) and an immunogenic composition comprising a linear polyribonucleotide as disclosed herein comprising a sequence encoding a coronavirus immunogen. A vaccine can be any bacterial infection vaccine or viral infection vaccine. In a particular embodiment, a vaccine is a pneumococcal polysaccharide vaccine, such as PCV13 or PPSV23. In some embodiments, the vaccine is an influenza vaccine. In some embodiments, the vaccine is an RSV vaccine (e.g., palivizumap).

### Production and Purification of Antibodies

Immunization of a subject with a polyribonucleotide described herein (e.g., a polyribonucleotide encoding a coronavirus immunogen) may induce the production of antibodies in the subject that bind to the immunogen expressed from the circular polyribonucleotide (e.g., produce anti-coronavirus antibodies). In some embodiments, immunization is for the purpose of producing antibodies in the subject (e.g., a human or a non-human animal) which are quantified or purified from the subject (e.g., for diagnostic or therapeutic use). Thereby, circular polyribonucleotides of the present invention may be used in methods of producing polyclonal or monoclonal antibodies (e.g., polyclonal or monoclonal anti-coronavirus antibodies).

For example, the disclosure provides administering a circular polyribonucleotide described herein (e.g., encoding a coronavirus immunogen) to a non-human animal (e.g., a non-human mammal, such as a goat, pig, rabbit, rat, mouse, llama, camel, horse, donkey, or bovine (cow)). The circular polyribonucleotide may be administered according to any composition, formulation, route or administration, amount, or dosing regimen described herein (e.g., optionally with an adjuvant, administered in the same composition or as part of a dosing regimen). In some embodiments, the non-human animal has a humanized immune system (e.g., a bovine having a humanized immune system).

Plasma including polyclonal antibodies produced from immunogenic compositions including circular polyribonucleotides as disclosed herein can be collected from a subject that was immunized with the circular polyribonucleotide. These polyclonal antibodies can be quantified (e.g., for diagnostic purposes in a human subject) or purified (e.g., for use in a method of treatment or for the development of monoclonal antibodies). Plasma can be collected by methods known to those of skill in the art, e.g., via plasmapheresis. Plasma can be collected from the same subject once or multiple times, for example, multiple times over a given period of time after an immunization, multiple times after an immunization, multiple times in between immunizations, or any combination thereof.

Antibodies, or fragments thereof, (e.g., polyclonal antibodies, such as human or humanized polyclonal antibodies) that bind specifically to a coronavirus immunogen (e.g., a coronavirus immunogen described herein) may be produced by the methods described herein. Antibodies, or fragments thereof, may be purified from blood (e.g., from blood plasma or blood serum) by methods known to those of skill in the art.

Polyclonal antibodies may be purified from plasma using techniques well known to those of skill in the art. For example, plasma is pH-adjusted to 4.8 (e.g., with dropwise addition of 20% acetic acid), fractionated by caprylic acid at a caprylic acid/total protein ratio of 1.0, and then clarified by centrifugation (e.g., at 10,000 g for 20 min at room temperature). The supernatant containing polyclonal antibodies (e.g., IgG polyclonal antibodies) is neutralized to pH 7.5 with 1 M tris, 0.22 μM filtered, and affinity-purified with an anti-human immunoglobulin-specific column (e.g., anti-human IgG light chain-specific column). The polyclonal antibodies are further purified by passage over an affinity column that specifically binds impurities, for example, non-human antibodies from the non-human animal. The polyclonal antibodies are stored in a suitable buffer, for example, a sterile-filtered buffer consisting of 10 mM glutamic acid monosodium salt, 262 mM D-sorbitol, and Tween (0.05 mg/ml) (pH 5.5). The quantity and concentration of the purified polyclonal antibodies are determined. HPLC size exclusion chromatography is conducted to determine whether aggregates or multimers are present. In some embodiments, the human polyclonal antibodies are purified from a non-human animal having a humanized immune system according to Beigel, J H et al. (Lancet Infect. Dis., 18:410-418 (2018), including Supplementary appendix), which is herein incorporated by reference in its entirety.

The disclosure also provides methods of producing antibodies in a human subject, e.g., for therapeutic treatment and/or diagnosis. For example, the disclosure provides a method of quantifying a level of anti-coronavirus antibodies in a subject following administration of a circular polyribonucleotide or immunogenic composition described herein. Quantification may be performed by methods known in the art (e.g., performing an antibody titer), for example by obtaining a blood sample from the subject and quantifying the anti-coronavirus antibody level using standard techniques, such as an enzyme-linked immunoassay (ELISA). Antibodies may also be purified by methods known to those of skill in the art.

### Pharmaceutical Compositions

In some embodiments, the immunogenic compositions administered to a subject (e.g., a subject for immunization) is a pharmaceutical composition. The pharmaceutical compositions as contemplated by the current invention may also include a pharmaceutically acceptable excipient.

The disclosure also provides pharmaceutical compositions comprising a plurality of polyclonal antibodies or a polyclonal antibody preparation against coronavirus disclosed herein and a pharmaceutically acceptable excipient.

A pharmaceutically acceptable excipient can be a non-carrier excipient. A non-carrier excipient serves as a vehicle or medium for a composition, such as a circular polyribonucleotide as described herein. A non-carrier excipient serves as a vehicle or medium for a composition, such as a linear polyribonucleotide as described herein. Non-limiting examples of a non-carrier excipient include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof. A non-carrier excipient can be any one of the inactive ingredients approved by the United States Food and Drug Administration (FDA) and listed in the Inactive Ingredient Database that does not exhibit a cell-penetrating effect. Pharmaceutical compositions may optionally comprise one or more additional active substances, e.g., therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference).

A pharmaceutical composition of the disclosure can comprise polyclonal antibodies of the disclosure, a circular polyribonucleotide of the disclosure, or a combination thereof. A pharmaceutical composition of the disclosure can comprise polyclonal antibodies of the disclosure, a linear polyribonucleotide of the disclosure, or a combination thereof. A pharmaceutical composition of the disclosure can comprise polyclonal antibodies of the disclosure, a circular polyribonucleotide of the disclosure, a linear polyribonucleotide of the disclosure, or a combination thereof.

In some embodiments, pharmaceutical compositions provided herein are suitable for administration to humans. In some embodiments, pharmaceutical compositions (e.g., comprising a circular polyribonucleotide, a linear polyribonucleotide, or an immunogenic composition as described herein) provided herein are suitable for administration to a subject (e.g., a subject for immunization), wherein the subject is a human. In some embodiments, pharmaceutical compositions (e.g., comprising a plurality of polyclonal antibodies or a polyclonal antibody preparation as described herein) provided herein are suitable for administration to a subject (e.g., a subject for treatment), wherein the subject is a human.

In some embodiments, pharmaceutical compositions (e.g., comprising a circular polyribonucleotide, a linear polyribonucleotide, or an immunogenic composition as described herein) provided herein are suitable for administration to a subject (e.g., a subject for immunization), wherein the subject is a non-human animal, for example, suitable for veterinary use. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, any animals, such as humans and/or other primates; mammals, including commercially relevant mammals, e.g., pet and live-stock animals, such as cattle, pigs, horses, sheep, cats, dogs, mice, and/or rats; and/or birds, including commercially relevant birds such as parrots, poultry, chickens, ducks, geese, hens or roosters, and/or turkeys; zoo animals, e.g., a feline; non-mammal animals, e.g., reptiles, fish, amphibians, etc.

In some embodiments, pharmaceutical compositions (e.g., comprising a plurality of polyclonal antibodies or a polyclonal antibody preparation as described herein) provided herein are suitable for administration to a subject (e.g., a subject for treatment), wherein the subject is non-human animal, for example, suitable for veterinary use. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, any animals, such as humans and/or other primates; mammals, including commercially relevant mammals, e.g., pet and live-stock animals, such as cattle, pigs, horses, sheep, cats, dogs, mice, and/or rats; and/or birds, including commercially relevant birds such as parrots, poultry, chickens, ducks, geese, hens or roosters, and/or turkeys; zoo animals, e.g., a feline; non-mammal animals, e.g., reptiles, fish, amphibians, etc.

Subjects (e.g., subjects for immunization or subjects for treatment) to which administration of the pharmaceutical compositions is contemplated include any ungulates.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

In some embodiments, the reference criterion for the amount of linear polyribonucleotide molecules present in the preparation is the presence of no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 1 μg/ml, 10 μg/ml, 50 μg/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, or 2 mg/ml of linear polyribonucleotide molecules.

In some embodiments, the reference criterion for the amount of circular polyribonucleotide molecules present in the preparation is at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), 99.9% (w/w), or 100% (w/w) molecules of the total ribonucleotide molecules in the pharmaceutical preparation.

In some embodiments, the reference criterion for the amount of linear polyribonucleotide molecules present in the preparation is no more than 0.5% (w/w), 1% (w/w), 2% (w/w), 5% (w/w), 10% (w/w), 15% (w/w), 20% (w/w), 25% (w/w), 30% (w/w), 40% (w/w), 50% (w/w) linear polyribonucleotide molecules of the total ribonucleotide molecules in the pharmaceutical preparation.

In some embodiments, the reference criterion for the amount of nicked polyribonucleotide molecules present in the preparation is no more than 0.5% (w/w), 1% (w/w), 2% (w/w), 5% (w/w), 10% (w/w), or 15% (w/w) nicked polyribonucleotide molecules of the total ribonucleotide molecules in the pharmaceutical preparation.

In some embodiments, the reference criterion for the amount of combined nicked and linear polyribonucleotide molecules present in the preparation is no more than 0.5% (w/w), 1% (w/w), 2% (w/w), 5% (w/w), 10% (w/w), 15% (w/w), 20% (w/w), 25% (w/w), 30% (w/w), 40% (w/w), 50% (w/w) combined nicked and linear polyribonucleotide molecules of the total ribonucleotide molecules in the pharmaceutical preparation. In some embodiments, a pharmaceutical preparation is an intermediate pharmaceutical preparation of a final circular polyribonucleotide drug product. In some embodiments, a pharmaceutical preparation is a drug substance or active pharmaceutical ingredient (API). In some embodiments, a pharmaceutical preparation is a drug product for administration to a subject.

In some embodiments, a preparation of circular polyribonucleotides is (before, during or after the reduction of linear RNA) further processed to substantially remove DNA, protein contamination (e.g., cell protein such as a host cell protein or protein process impurities), endotoxin, mononucleotide molecules, and/or a process-related impurity.

Pharmaceutical compositions can be sterile and stable under the conditions of manufacture and storage. The composition can be formulated as a solution, microemulsion, liposome, or other ordered structure suitable to high drug concentration. Examples of suitable aqueous and non-aqueous compositions which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Sterile injectable solutions can be prepared by incorporating the active compound (e.g., an agent, such as a circular polyribonucleotide, linear polyribonucleotide, or antibody) in the required amount in an appropriate solvent with one or a combination of ingredients e.g., as enumerated above, as required, followed by sterilization microfiltration. Generally, dispersions are prepared by incorporating the active compound into a sterile vehicle that contains a basic dispersion medium and the required other ingredients e.g., from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Agents (e.g., circular polyribonucleotides, linear polyribonucleotides, or antibodies) of the disclosure can be prepared in a composition that will protect them against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Methods for the preparation of such formulations are generally known to those skilled in the art. See, e.g., Sustained and Controlled Release Drug Delivery Systems, J. R. Robinson, ed., Marcel Dekker, Inc., New York, 1978. A composition of the disclosure can be, for example, an immediate release form or a controlled release formulation. An immediate release formulation can be formulated to allow the compounds (e.g., agents, such as a circular polyribonucleotide, linear polyribonucleotide or antibody) to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

Pharmaceutical formulations for administration can include aqueous solutions of the active compounds (e.g., agents, such as a circular polyribonucleotide, linear polyribonucleotide, or antibody) in water soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the agents to allow for the preparation of highly concentrated solutions. The active ingredient can be in powder form for constitution with a suitable vehicle, for example, sterile pyrogen-free water, before use.

Methods for the preparation of compositions comprising the agents described herein include formulating the agents with one or more inert, pharmaceutically acceptable excipients or carriers to form a solid, semi-solid, or liquid composition. Solid compositions include, for example, powders, dispersible granules, and cachets. Liquid compositions include, for example, solutions in which an agent is dissolved, emulsions comprising an agent, or a solution containing liposomes, micelles, or nanoparticles comprising an agent as disclosed herein. Semi-solid compositions include, for example, gels, suspensions, and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically acceptable additives.

Non-limiting examples of dosage forms suitable for use in the disclosure include liquid, powder, gel, nanosuspension, nanoparticle, microgel, aqueous or oily suspensions, emulsion, and any combination thereof.

In some embodiments, a formulation of the disclosure contains a thermal stabilizer, such as a sugar or sugar alcohol, for example, sucrose, sorbitol, glycerol, trehalose, or mannitol, or any combination thereof. In some embodiments, the stabilizer is a sugar. In some embodiments, the sugar is sucrose, mannitol, or trehalose.

Pharmaceutical compositions as described herein can be formulated for example to include a pharmaceutical excipient or carrier. A pharmaceutical carrier may be a membrane, lipid bilayer, and/or a polymeric carrier, e.g., a liposome or particle such as a nanoparticle, e.g., a lipid nanoparticle, and delivered by known methods, such as via partial or full encapsulation of the circular polyribonucleotide, to a subject (e.g., a subject for immunization or a subject for treatment) in need thereof (e.g., a human or non-human agricultural or domestic animal, e.g., cattle, dog, cat, horse, poultry).

### Methods of Delivery

The circular polyribonucleotide as described herein, or a pharmaceutical composition thereof as described herein can be administered to a cell in a vesicle or other membrane-based carrier as described herein. The linear polyribonucleotide as described herein, or a pharmaceutical composition thereof as described herein can be administered to a cell in a vesicle or other membrane-based carrier as described herein.

In some embodiments, the cell is a eukaryotic cell. In some embodiments, the cell is a mammalian cell. In some embodiments, the cell is an ungulate cell. In some embodiments, the cell is an animal cell. In some embodiments, the cell is an immune cell. In some embodiments, the tissue is a connective tissue, a muscle tissue, a nervous tissue, or an epithelial tissue. In some embodiments, the tissue is an organ (e.g., liver, lung, spleen, kidney, etc.). In some embodiments, the subject (e.g., a subject for immunization) is a mammal. In some embodiments, the subject (e.g., a subject for immunization) is an ungulate.

In some embodiments, a pharmaceutical formulation disclosed herein can comprise: (i) a compound (e.g., circular polyribonucleotide or antibody) disclosed herein; (ii) a buffer; (iii) a non-ionic detergent; (iv) a tonicity agent; and (v) a stabilizer. In some embodiments, a pharmaceutical formulation disclosed herein can comprise: (i) a compound (e.g., linear polyribonucleotide or antibody) disclosed herein; (ii) a buffer; (iii) a non-ionic detergent; (iv) a tonicity agent; and (v) a stabilizer. In some embodiments, the pharmaceutical formulation disclosed herein is a stable liquid pharmaceutical formulation.

### Diluents

In some embodiments, an immunogenic composition of the invention comprises a circular polyribonucleotide and a diluent. In some embodiments, an immunogenic composition of the invention comprises a linear polyribonucleotide and a diluent.

A diluent can be a non-carrier excipient. A non-carrier excipient serves as a vehicle or medium for a composition, such as a polyribonucleotide as described herein. Non-limiting examples of a non-carrier excipient include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof. A non-carrier excipient can be any one of the inactive ingredients approved by the United States Food and Drug Administration (FDA) and listed in the Inactive Ingredient Database that does not exhibit a cell-penetrating effect. A non-carrier excipient can be any inactive ingredient suitable for administration to a non-human animal, for example, suitable for veterinary use. Modification of compositions suitable for administration to humans in order to render the compositions suitable for administration to various animals is well understood, and the ordinarily skilled veterinary pharmacologist can design and/or perform such modification with merely ordinary, if any, experimentation.

In some embodiments, the polyribonucleotide may be delivered as a naked delivery formulation, such as including a diluent. A naked delivery formulation delivers a polyribonucleotide, to a cell without the aid of a carrier and without modification or partial or complete encapsulation of the polyribonucleotide, capped polyribonucleotide, or complex thereof.

A naked delivery formulation is a formulation that is free from a carrier and wherein the polyribonucleotide is without a covalent modification that binds a moiety that aids in delivery to a cell or without partial or complete encapsulation of the polyribonucleotide. In some embodiments, a polyribonucleotide without a covalent modification that binds a moiety that aids in delivery to a cell is a polyribonucleotide that is not covalently bound to a protein, small molecule, a particle, a polymer, or a biopolymer. A polyribonucleotide without covalent modification that binds a moiety that aids in delivery to a cell does not contain a modified phosphate group. For example, a polyribonucleotide without a covalent modification that binds a moiety that aids in delivery to a cell does not contain phosphorothioate, phosphoroselenates, boranophosphates, boranophosphate esters, hydrogen phosphonates, phosphoramidates, phosphorodiamidates, alkyl or aryl phosphonates, or phosphotriesters.

In some embodiments, a naked delivery formulation is free of any or all transfection reagents, cationic carriers, carbohydrate carriers, nanoparticle carriers, or protein carriers. In some embodiments, a naked delivery formulation is free from phytoglycogen octenyl succinate, phytoglycogen beta-dextrin, anhydride-modified phytoglycogen beta-dextrin, lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, I,2-Dioleoyl-3-Trimethylammonium-Propane (DOTAP), N-[1-(2,3-dioleoyloxy)propyl]-N,N,N-trimethylammonium chloride (DOTMA), I-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-I-propanaminium trifluoroacetate (DOSPA), 3B—[N— (N\N′-Dimethylaminoethane)-carbamoyl]Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride (DODAC), human serum albumin (HSA), low-density lipoprotein (LDL), high-density lipoprotein (HDL), or globulin.

In certain embodiments, a naked delivery formulation includes a non-carrier excipient. In some embodiments, a non-carrier excipient includes an inactive ingredient that does not exhibit a cell-penetrating effect. In some embodiments, a non-carrier excipient includes a buffer, for example PBS. In some embodiments, a non-carrier excipient is a solvent, a non-aqueous solvent, a diluent, a suspension aid, a surface-active agent, an isotonic agent, a thickening agent, an emulsifying agent, a preservative, a polymer, a peptide, a protein, a cell, a hyaluronidase, a dispersing agent, a granulating agent, a disintegrating agent, a binding agent, a buffering agent, a lubricating agent, or an oil.

In some embodiments, a naked delivery formulation includes a diluent. A diluent may be a liquid diluent or a solid diluent. In some embodiments, a diluent is an RNA solubilizing agent, a buffer, or an isotonic agent. Examples of an RNA solubilizing agent include water, ethanol, methanol, acetone, formamide, and 2-propanol. Examples of a buffer include 2-(N-morpholino)ethanesulfonic acid (MES), Bis-Tris, 2-[(2-amino-2-oxoethyl)-(carboxymethyl)amino]acetic acid (ADA), N-(2-Acetamido)-2-aminoethanesulfonic acid (ACES), piperazine-N,N′-bis(2-ethanesulfonic acid) (PIPES), 2-[[1,3-dihydroxy-2-(hydroxymethyl)propan-2-yl]amino]ethanesulfonic acid (TES), 3-(N-morpholino)propanesulfonic acid (MOPS), 4-(2-hydroxyethyl)-1-piperazineethanesulfonic acid (HEPES), Tris, Tricine, Gly-Gly, Bicine, or phosphate. Examples of an isotonic agent include glycerin, mannitol, polyethylene glycol, propylene glycol, trehalose, or sucrose.

In some embodiments, the formulation includes a cell-penetrating agent. In some embodiments, the formulation is a topical formulation and includes a cell-penetrating agent. The cell-penetrating agent can include organic compounds such as alcohols having one or more hydroxyl function groups. In some cases, the cell-penetrating agent includes an alcohol such as, but not limited to, monohydric alcohols, polyhydric alcohols, unsaturated aliphatic alcohols, and alicyclic alcohols. The cell-penetrating agent can include one or more of methanol, ethanol, isopropanol, phenoxyethanol, triethanolamine, phenethyl alcohol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, or hydroxyethylcellulose. In certain embodiments, the cell-penetrating agent includes ethanol. The cell-penetrating agents can include any cell-penetrating agent in any amount or in any formulation as described in WO 2020/180751 or WO 2020/180752, which are hereby incorporated by reference in their entirety.

### Carriers

In some embodiments, an immunogenic composition of the invention comprises a circular polyribonucleotide and a carrier. In some embodiments, an immunogenic composition of the invention comprises a linear polyribonucleotide and a carrier.

In certain embodiments, an immunogenic composition comprises a circular polyribonucleotide as described herein in a vesicle or other membrane-based carrier. In certain embodiments, an immunogenic composition comprises a linear polyribonucleotide as described herein in a vesicle or other membrane-based carrier.

In other embodiments, an immunogenic composition includes the polyribonucleotide in or via a cell, vesicle, or other membrane-based carrier. In one embodiment, an immunogenic composition includes the polyribonucleotide in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral, or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, JOURNAL OF DRUG DELIVERY, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, JOURNAL OF DRUG DELIVERY, vol. 2011, Article ID 469679, 12 pages, 2011. doi:10.1155/2011/469679 for review). Extruded lipids can be prepared by extruding through filters of decreasing size, as described in Templeton et al., NATURE BIOTECH, 15:647-52, 1997, the teachings of which relating to extruded lipid preparation are incorporated herein by reference.

In certain embodiments, an immunogenic composition of the disclosure includes a polyribonucleotide and lipid nanoparticles, for example lipid nanoparticles described herein. Lipid nanoparticles are another example of a carrier that provides a biocompatible and biodegradable delivery system for a polyribonucleotide molecule as described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Additional non-limiting examples of carriers include carbohydrate carriers (e.g., an anhydride-modified phytoglycogen or glycogen-type material), protein carriers (e.g., a protein covalently linked to the polyribonucleotide), or cationic carriers (e.g., a cationic lipopolymer or transfection reagent). Non-limiting examples of carbohydrate carriers include phytoglycogen octenyl succinate, phytoglycogen beta-dextrin, and anhydride-modified phytoglycogen beta-dextrin. Non-limiting examples of cationic carriers include lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane (DOTAP), N-[1-(2,3-dioleoyloxy)propyl]-N,N,N-trimethylammonium chloride (DOTMA), I-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-I-propanaminium trifluoroacetate (DOSPA), 3B—[N— (N\N′-Dimethylaminoethane)-carbamoyl]Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), and N,N-dioleyl-N,N-dimethylammonium chloride (DODAC). Non-limiting examples of protein carriers include human serum albumin (HSA), low-density lipoprotein (LDL), high-density lipoprotein (HDL), or globulin.

Exosomes can also be used as drug delivery vehicles for an RNA composition or preparation described herein. For a review, see Ha et al. July 2016. ACTA PHARMACEUTICA SINICA B. Volume 6, Issue 4, Pages 287-296; https://doi.org/10.1016/j.apsb.2016.02.001.

Ex vivo differentiated red blood cells can also be used as a carrier for an RNA composition or preparation described herein. See, e.g., International Patent Publication Nos. WO2015/073587; WO2017/123646; WO2017/123644; WO2018/102740; WO2016/183482; WO2015/153102; WO2018/151829; WO2018/009838; Shi et al. 2014. Proc Natl Acad Sci USA. 111(28): 10131-10136; U.S. Pat. No. 9,644,180; Huang et al. 2017. NATURE COMMUNICATIONS 8: 423; Shi et al. 2014. PROC NATL ACAD SCI USA. 111(28): 10131-136.

Fusosome compositions, e.g., as described in International Patent Publication No. WO2018/208728, can also be used as carriers to deliver a polyribonucleotide molecule described herein.

Virosomes and virus-like particles (VLPs) can also be used as carriers to deliver a polyribonucleotide molecule described herein to targeted cells.

Plant nanovesicles and plant messenger packs (PMPs), e.g., as described in International Patent Publication Nos. WO2011/097480, WO2013/070324, WO2017/004526, or WO2020/041784 can also be used as carriers to deliver the RNA composition or preparation described herein.

Microbubbles can also be used as carriers to deliver a polyribonucleotide molecule described herein. See, e.g., U.S. Pat. No. 7,115,583; Beeri, R. et al., CIRCULATION. 2002 Oct. 1; 106(14):1756-59; Bez, M. et al., NAT PROTOC. 2019 April; 14(4): 1015-26; Hernot, S. et al., ADV DRUG DELIV REV. 2008 Jun. 30; 60(10): 1153-66; Rychak, J. J. et al., ADV DRUG DELIV REV. 2014 June; 72: 82-93. In some embodiments, microbubbles are albumin-coated perfluorocarbon microbubbles.

The carrier including the polyribonucleotides described herein may include a plurality of particles. The particles may have median article size of 30 to 700 nanometers (e.g., 30 to 50, 50 to 100, 100 to 200, 200 to 300, 300 to 400, 400 to 500, 500 to 600, 600 to 700, 100 to 500, 50 to 500, or 200 to 700 nanometers). The size of the particle may be optimized to favor deposition of the payload, including the polyribonucleotide into a cell. Deposition of the polyribonucleotide into certain cell types may favor different particle sizes. For example, the particle size may be optimized for deposition of the polyribonucleotide into immunogen presenting cells. The particle size may be optimized for deposition of the polyribonucleotide into dendritic cells. Additionally, the particle size may be optimized for depositions of the polyribonucleotide into draining lymph node cells.

### Lipid Nanoparticles

The compositions, methods, and delivery systems provided by the present disclosure may employ any suitable carrier or delivery modality described herein, including, in certain embodiments, lipid nanoparticles (LNPs). Lipid nanoparticles, in some embodiments, include one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; incorporated herein by reference in its entirety); one or more sterols (e.g., cholesterol).

Lipids that can be used in nanoparticle formations (e.g., lipid nanoparticles) include, for example those described in Table 4 of WO2019217941, which is incorporated by reference—e.g., a lipid-containing nanoparticle can include one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as I-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-I-O-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al. (2020), dx.doi.org/10.1021/acs.nanolett.0c01386, incorporated herein by reference.

In some embodiments, the lipid particle includes an ionizable lipid, a non-cationic lipid, a conjugated lipid that inhibits aggregation of particles, and a sterol. The amounts of these components can be varied independently and to achieve desired properties. For example, in some embodiments, the lipid nanoparticle includes an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid (e.g., RNA (e.g., circular polyribonucleotide, linear polyribonucleotide)) described herein includes,

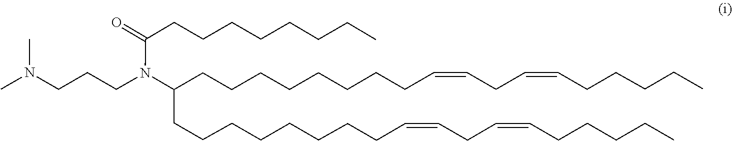 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP including Formula (i) is used to deliver a polyribonucleotide (e.g., a circular polyribonucleotide, a linear polyribonucleotide) composition described herein to cells.

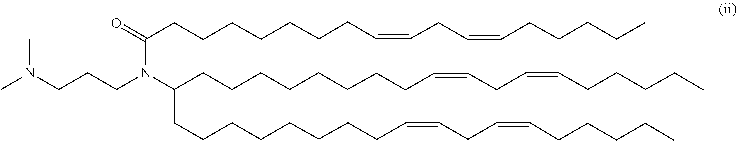 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP including Formula (ii) is used to deliver a polyribonucleotide (e.g., a circular polyribonucleotide, a linear polyribonucleotide) composition described herein to cells.

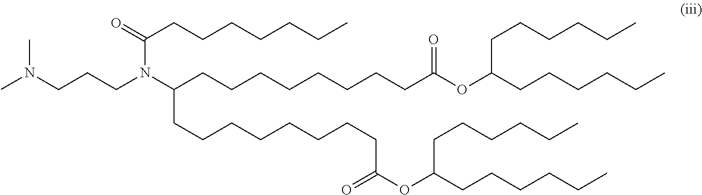 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP including Formula (iii) is used to deliver a polyribonucleotide (e.g., a circular polyribonucleotide, a linear polyribonucleotide) composition described herein to cells.

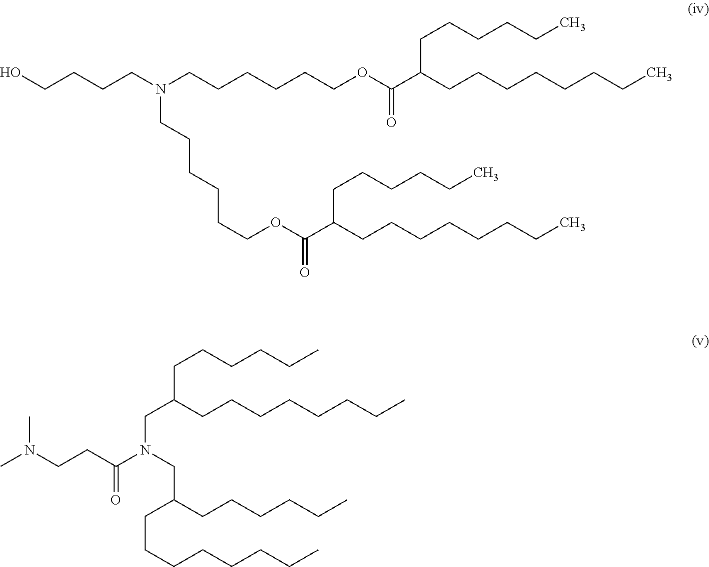

In some embodiments an LNP including Formula (v) is used to deliver a polyribonucleotide (e.g., a circular polyribonucleotide, a linear polyribonucleotide) composition described herein to cells.

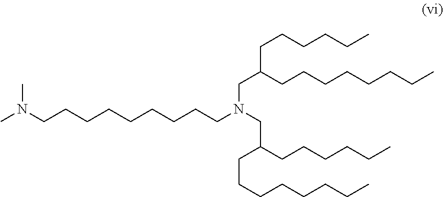 [SMILES: CCCCCCCCC(CCCCCC)CN(CCCCCCCCCN(C)C)CC(CCCCCC)CCCCCCCC]

In some embodiments an LNP including Formula (vi) is used to deliver a polyribonucleotide (e.g., a circular polyribonucleotide, a linear polyribonucleotide) composition described herein to cells.

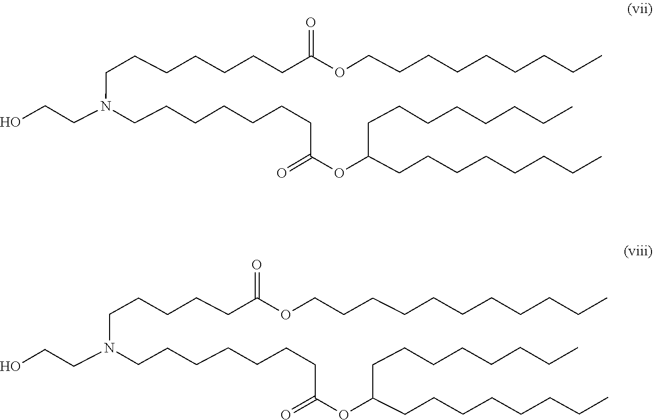 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP including Formula (viii) is used to deliver a polyribonucleotide (e.g., a circular polyribonucleotide, a linear polyribonucleotide) composition described herein to cells.

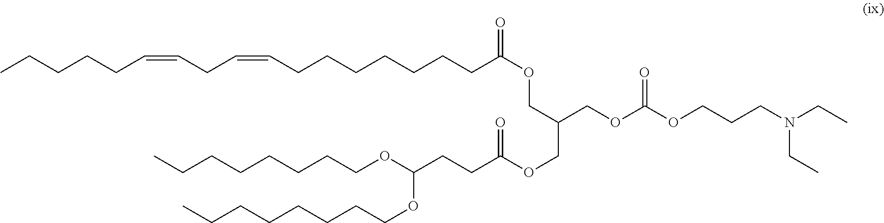 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments an LNP including Formula (ix) is used to deliver a polyribonucleotide (e.g., a circular polyribonucleotide, a linear polyribonucleotide) composition described herein to cells.

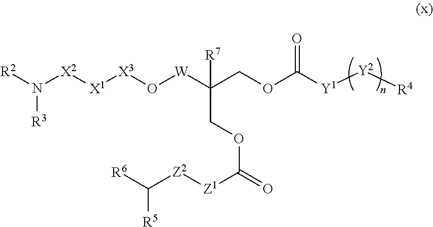

wherein
X¹ is O, NR¹, or a direct bond, X² is C2-5 alkylene, X³ is C(═O) or a direct bond, R¹ is H or Me, R³ is C₁₋₃ alkyl, R² is C₁₋₃ alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene, Y² is selected from

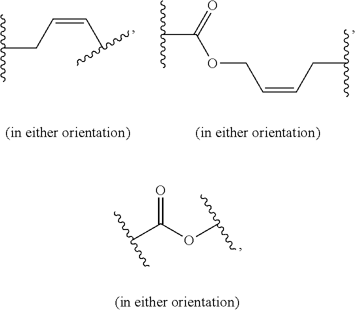 [SMILES: */C=C\C* ; *C/C=C\COC(*)=O ; *OC(*)=O]

n is 0 to 3, R⁴ is C₁₋₁₅ alkyl, Z¹ is C₁₋₆ alkylene or a direct bond,
Z² is 0

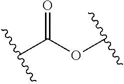 [SMILES: *OC(*)=O]

(in either orientation) or absent, provided that if Z¹ is a direct bond, Z² is absent;
R⁵ is C5-9 alkyl or C6-10 alkoxy, R⁶ is C5-9 alkyl or C6-10 alkoxy, W is methylene or a direct bond, and
R⁷ is H or Me, or a salt thereof, provided that if R³ and R² are C2 alkyls, X¹ is O, X² is linear C3 alkylene,
X³ is C(═O), Y¹ is linear Ce alkylene, (Y²)n-R⁴ is

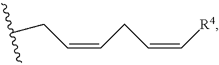

R⁴ is linear C5 alkyl, Z¹ is C2 alkylene, Z² is absent, W is methylene, and R⁷ is H, then R⁵ and R⁶ are not Cx alkoxy.

In some embodiments an LNP including Formula (xii) is used to deliver a polyribonucleotide (e.g., a circular polyribonucleotide, a linear polyribonucleotide) composition described herein to cells.

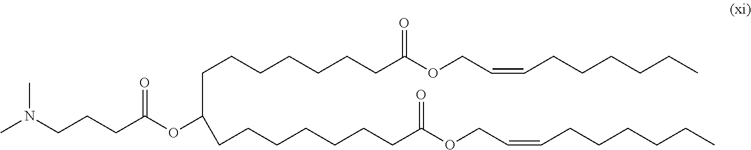 [SMILES: CCCCCC/C=C\COC(=O)CCCCCCCC(CCCCCCCC(=O)OC/C=C\CCCCCC)OC(=O)CCCN(C)C]

In some embodiments an LNP including Formula (xi) is used to deliver a polyribonucleotide (e.g., a circular polyribonucleotide, a linear polyribonucleotide) composition described herein to cells.

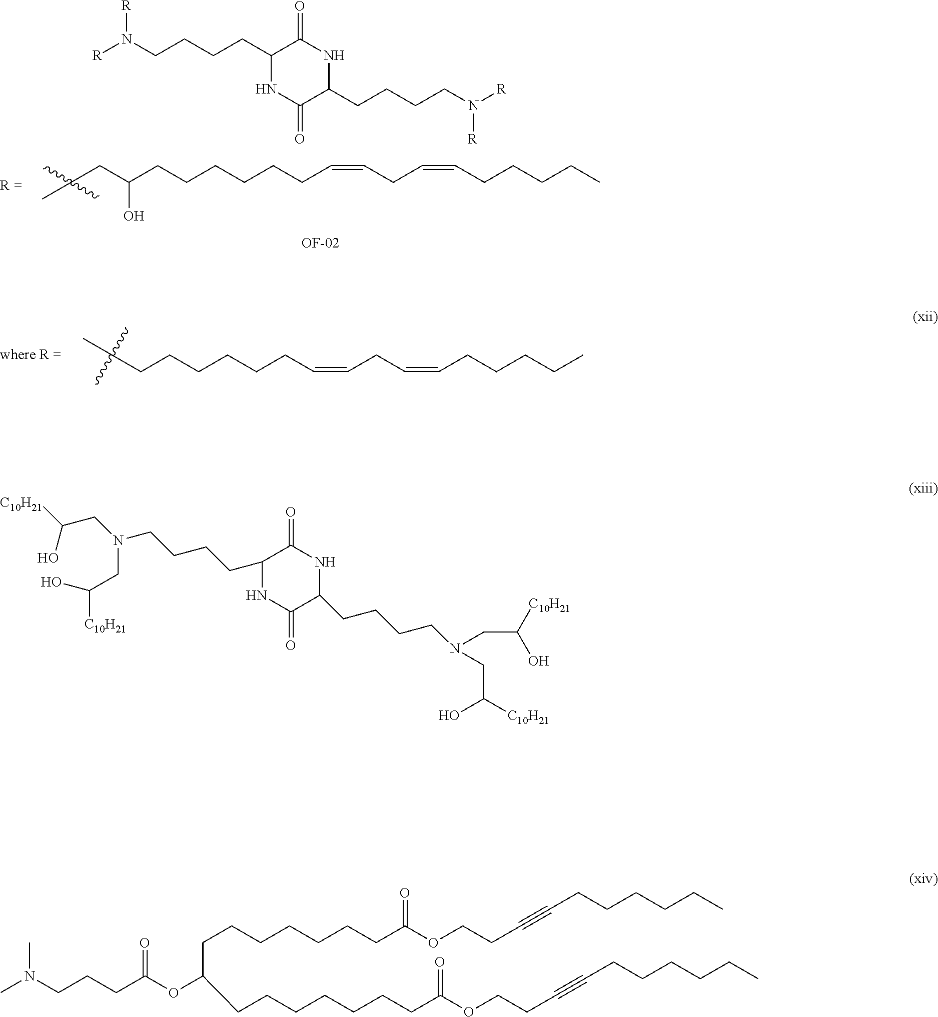

In some embodiments an LNP includes a compound of Formula (xiii) and a compound of Formula (xiv).

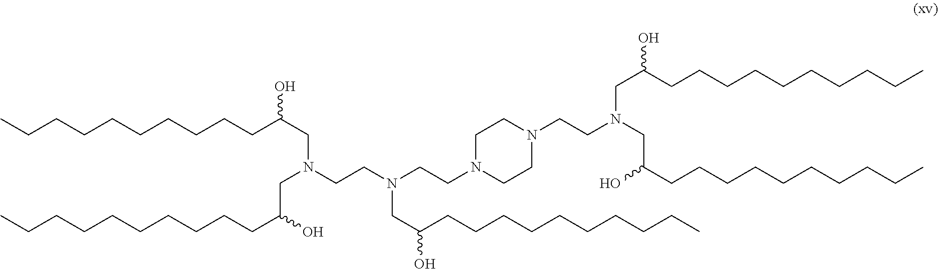 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments an LNP including Formula (xv) is used to deliver a polyribonucleotide (e.g., a circular polyribonucleotide, a linear polyribonucleotide) composition described herein to cells.

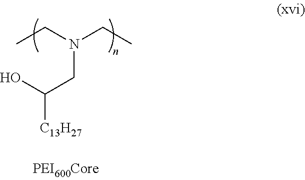

In some embodiments an LNP including a formulation of Formula (xvi) is used to deliver a polyribonucleotide (e.g., a circular polyribonucleotide, a linear polyribonucleotide) composition described herein to cells.

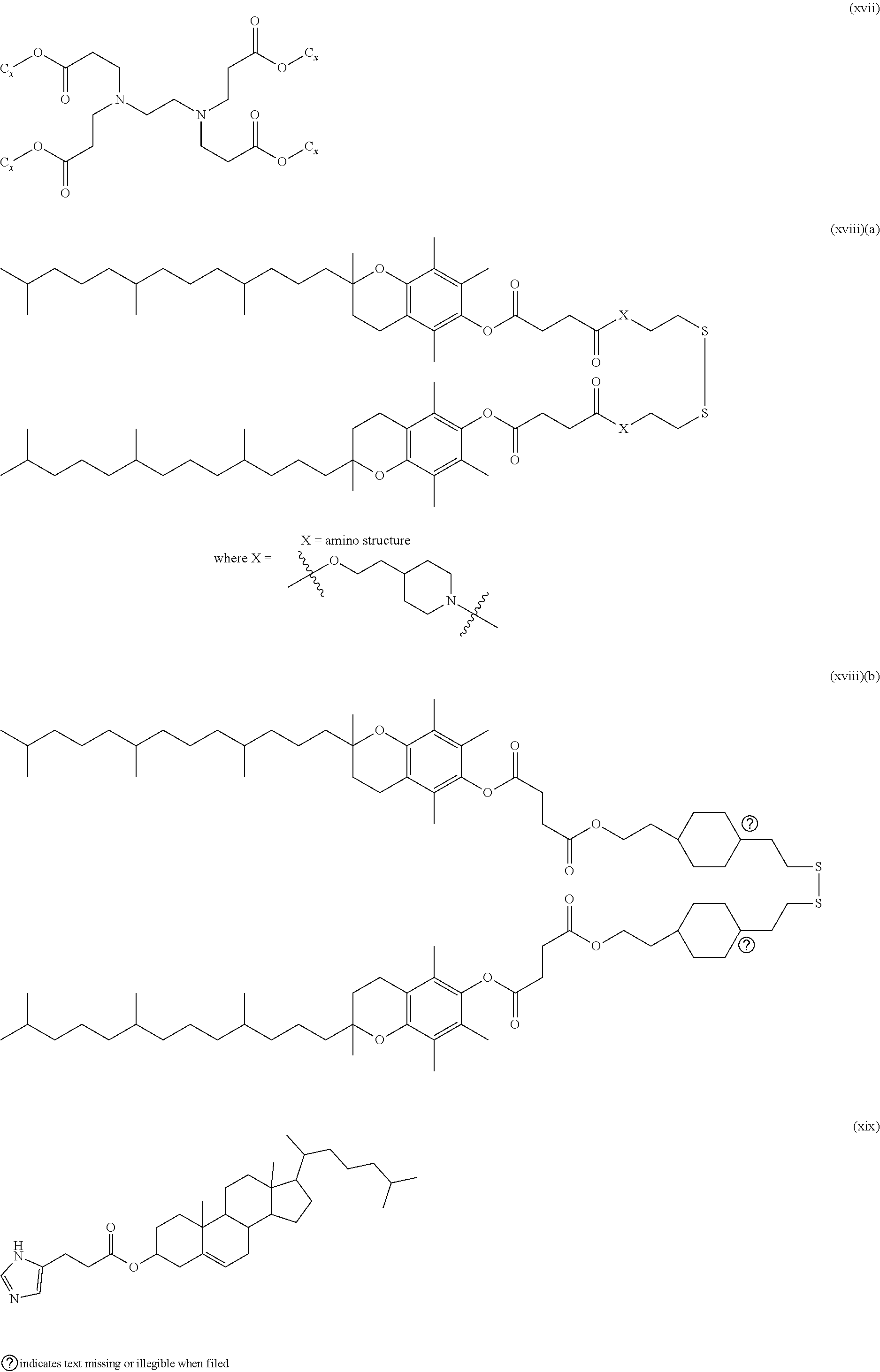

In some embodiments, a lipid compound used to form lipid nanoparticles for the delivery of compositions described herein, e.g., nucleic acid (e.g., RNA (e.g., circular polyribonucleotide, linear polyribonucleotide)) described herein is made by one of the following reactions:

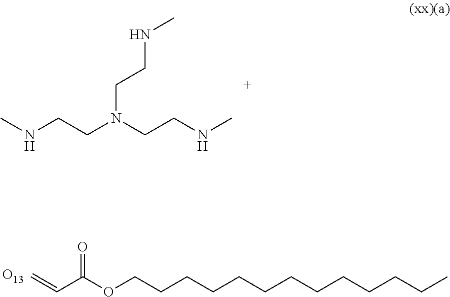

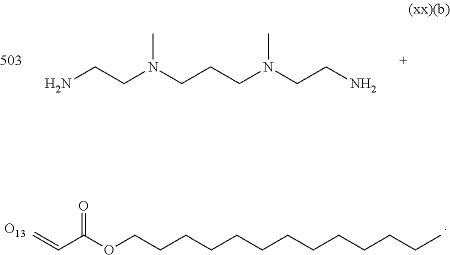 [SMILES: CN(CCN)CCCN(C)CCN ; "O13" ; C=CC(=O)OCCCCCCCCCCCCC]

In some embodiments an LNP including Formula (xxi) is used to deliver a polyribonucleotide (e.g., a circular polyribonucleotide, a linear polyribonucleotide) composition described herein to cells. In some embodiments the LNP of Formula (xxi) is an LNP described by WO2021113777 (e.g., a lipid of Formula (1) such as a lipid of Table 1 of WO2021113777).

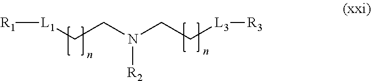

wherein
each n is independently an integer from 2-15; L₁ and L₃ are each independently —OC(O)—* or —C(O)O—*, wherein “*” indicates the attachment point to R₁ or R₃.

R₁ and R₃ are each independently a linear or branched C9-C20 alkyl or C9-C20 alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkyl sulfonyl, and alkyl sulfonealkyl; and
R₂ is selected from a group consisting of:

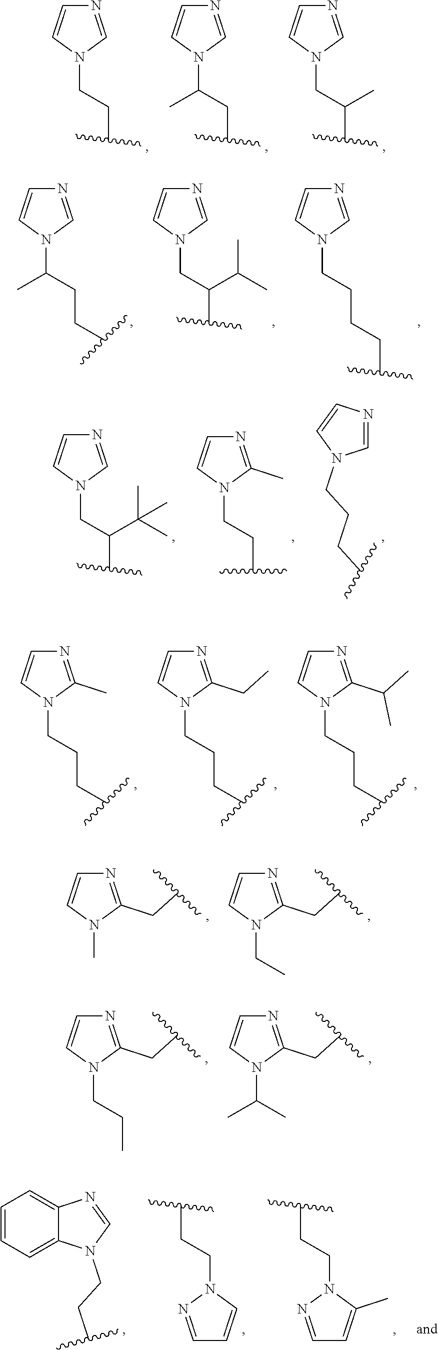

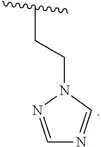 [SMILES: *CCn1cncn1]

In some embodiments an LNP including Formula (xxii) is used to deliver a polyribonucleotide (e.g., a circular polyribonucleotide, a linear polyribonucleotide) composition described herein to cells. In some embodiments the LNP of Formula (xxii) is an LNP described by WO2021113777 (e.g., a lipid of Formula (2) such as a lipid of Table 2 of WO2021113777).

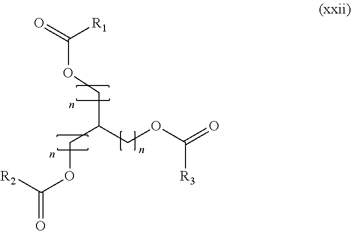

wherein
each n is independently an integer from 1-15;
R₁ and R₂ are each independently selected from a group consisting of:

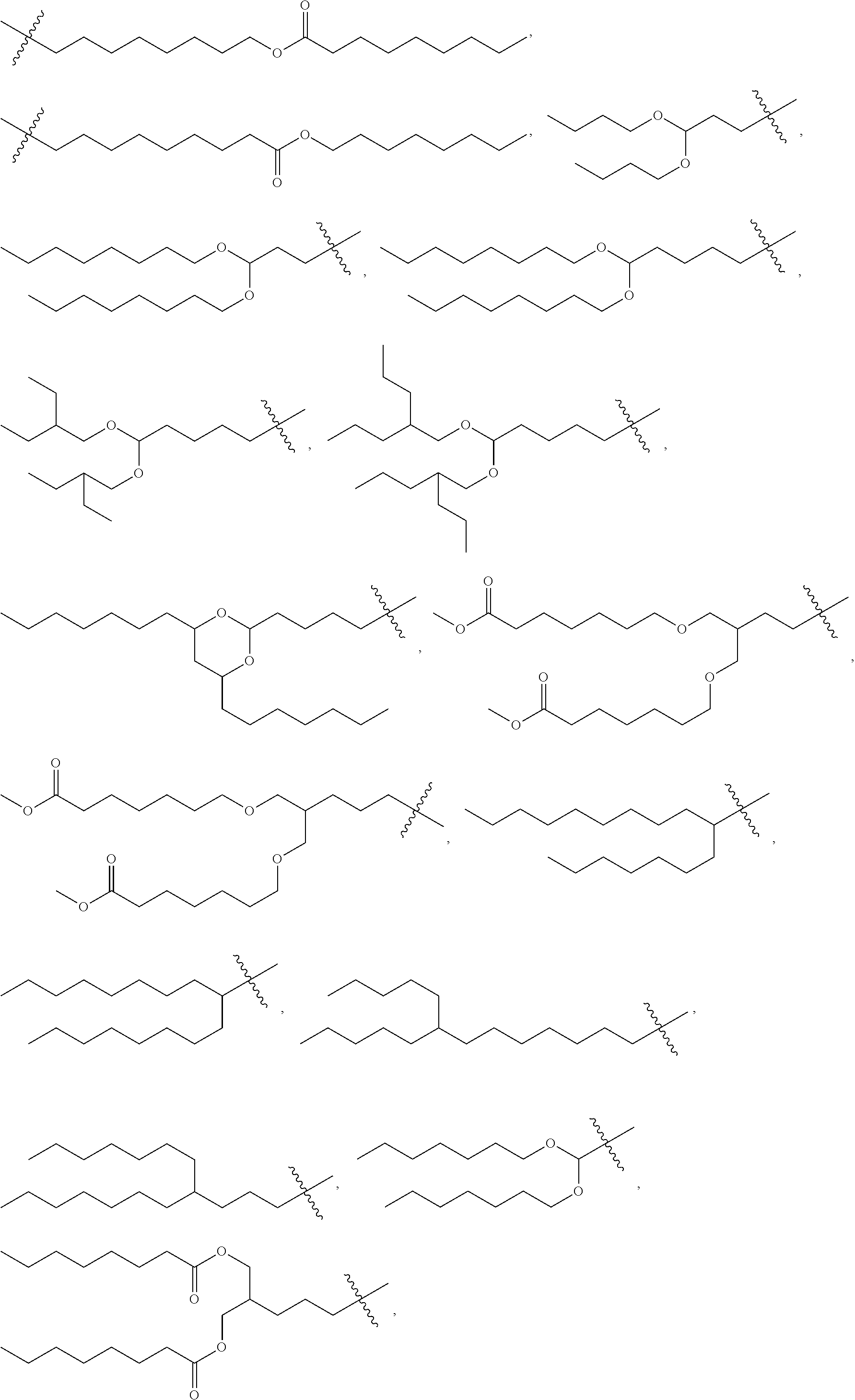

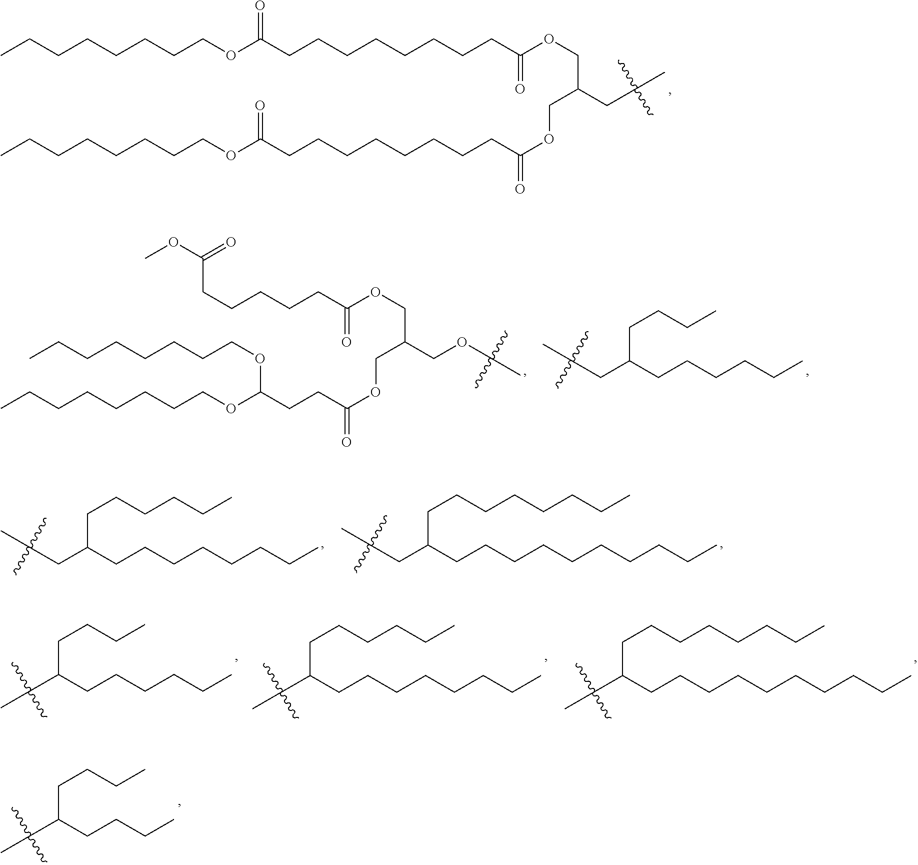

R₃ is selected from a group consisting of:

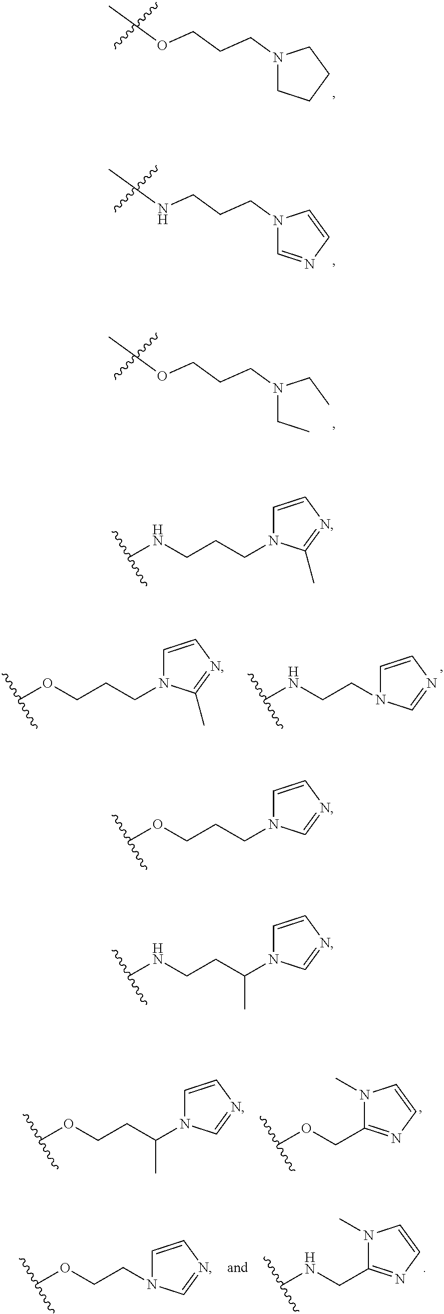

In some embodiments an LNP including Formula (xxiii) is used to deliver a polyribonucleotide (e.g., a circular polyribonucleotide, a linear polyribonucleotide) composition described herein to cells. In some embodiments the LNP of Formula (xxiii) is an LNP described by WO2021113777 (e.g., a lipid of Formula (3) such as a lipid of Table 3 of WO2021113777).

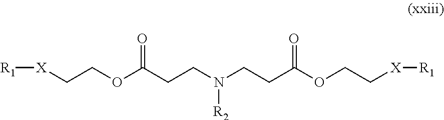

wherein
X is selected from —O—, —S—, or —OC(O)—*, wherein * indicates the attachment point to R₁;
R is selected from a group consisting of;

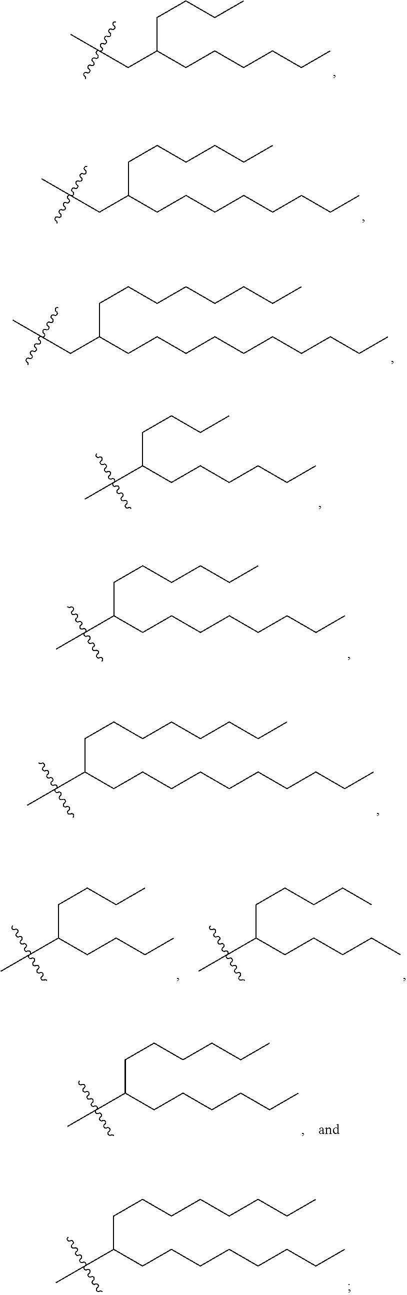

and R₂ is selected from a group consisting of:

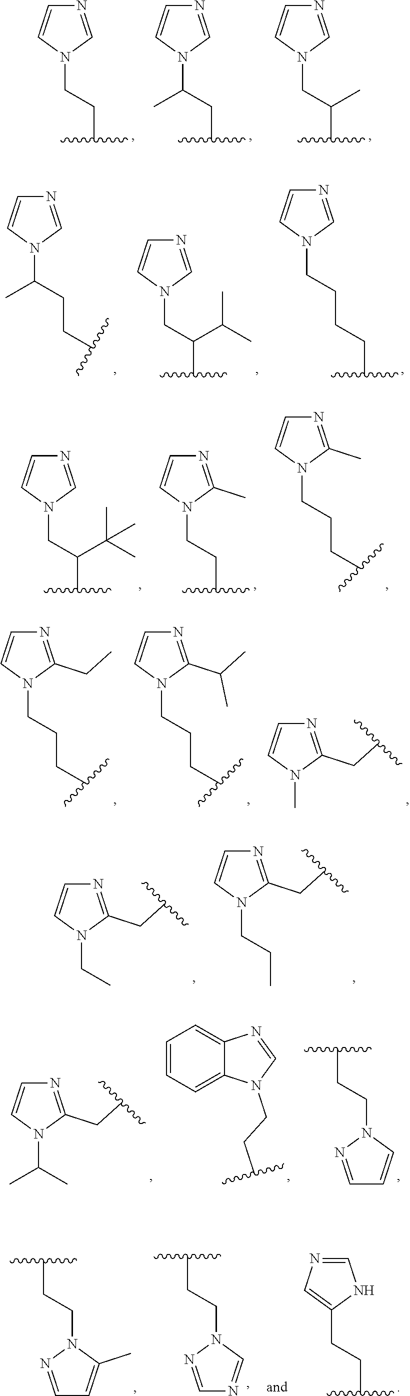

In some embodiments, a composition described herein (e.g., a nucleic acid (e.g., a circular polyribonucleotide, a linear polyribonucleotide) or a protein) is provided in an LNP that includes an ionizable lipid. In some embodiments, the ionizable lipid is heptadecan-9-yl 8-((2-hydroxyethyl) (6-oxo-6-(undecyloxy)hexyl)amino)octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LP01), e.g., as synthesized in Example 13 of WO2015/095340 (incorporated by reference herein in its entirety). In some embodiments, the ionizable lipid is Di((Z)-non-2-en-1-yl) 9-((4-dimethylamino) butanoyl)oxy) heptadecanedioate (L319), e.g., as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-IH-cyclopenta[a]phenanthren-3-yl 3-(1H-imidazol-4-yl) propanoate, e.g., Structure (I) from WO2020/106946 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle includes a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol, and polymer conjugated lipids. In some embodiments, the cationic lipid may be an ionizable cationic lipid. An exemplary cationic lipid as disclosed herein may have an effective pKa over 6.0. In embodiments, a lipid nanoparticle may include a second cationic lipid having a different effective pKa (e.g., greater than the first effective pKa), than the first cationic lipid. A lipid nanoparticle may include between 40 and 60 mol percent of a cationic lipid, a neutral lipid, a steroid, a polymer conjugated lipid, and a therapeutic agent, e.g., a nucleic acid (e.g., RNA (e.g., a circular polyribonucleotide, a linear polyribonucleotide)) described herein, encapsulated within or associated with the lipid nanoparticle. In some embodiments, the nucleic acid is co-formulated with the cationic lipid. The nucleic acid may be adsorbed to the surface of an LNP, e.g., an LNP including a cationic lipid. In some embodiments, the nucleic acid may be encapsulated in an LNP, e.g., an LNP including a cationic lipid. In some embodiments, the lipid nanoparticle may include a targeting moiety, e.g., coated with a targeting agent. In embodiments, the LNP formulation is biodegradable. In some embodiments, a lipid nanoparticle including one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of an RNA molecule.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; I-5 or I-8 of WO2020/081938; 18 or 25 of U.S. Pat. No. 9,867,888; A of US2019/0136231; II of WO2020/219876; I of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946; and (1), (2), (3), or (4) of WO2021/113777. Exemplary lipids further include a lipid of any one of Tables 1-16 of WO2021/113777.

In some embodiments, the ionizable lipid is MC3 (6Z,9Z,28Z,3 IZ)-heptatriaconta-6,9,28,31-tetraen-I9-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-I3,I6-dien-I-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), I8-I-trans PE, I-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can include, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not include any phospholipids.

In some aspects, the lipid nanoparticle can further include a component, such as a sterol, to provide membrane integrity. One exemplary sterol that can be used in the lipid nanoparticle is cholesterol and derivatives thereof. Non-limiting examples of cholesterol derivatives include polar analogues such as 5α-cholestanol, 53-coprostanol, cholesteryl-(2-hydroxy)-ethyl ether, cholesteryl-(4′-hydroxy)-butyl ether, and 6-ketocholestanol; non-polar analogues such as 5α-cholestane, cholestenone, 5α-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can include 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can include a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as I-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-I-O-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, US2018/0028664, and WO2017/099823, the contents of all of which are incorporated herein by reference in their entirety. In some embodiments, a PEG-lipid is a compound of Formula III, III-a-1, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (I-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid includes PEG-DMG, 1,2-dimyristoyl-sn-glycero-3-phosphoethanolamine-N-[methoxy(polyethylene glycol)-2000]. In some embodiments, the PEG-lipid includes a structure selected from:

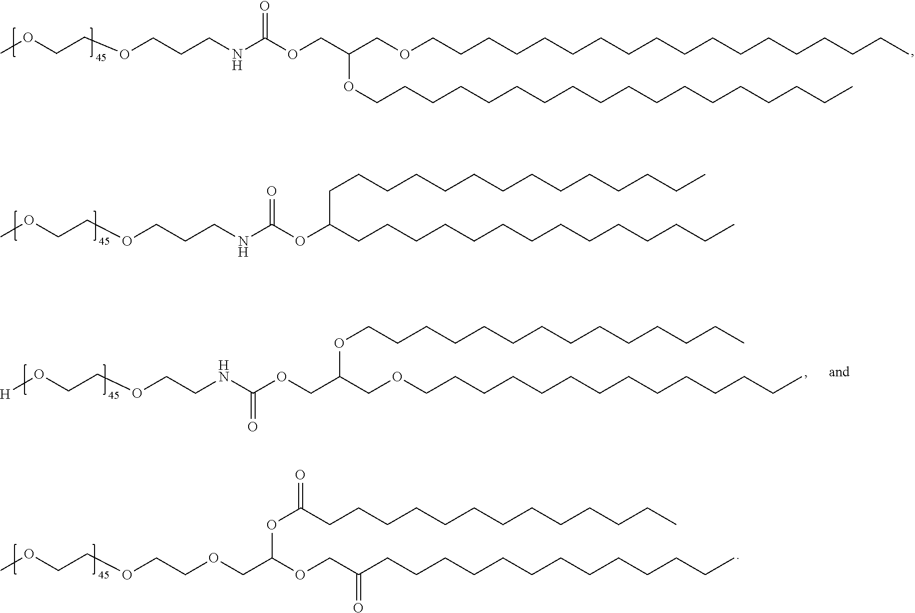

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9, the contents of all of which are incorporated herein by reference in their entirety.

In some embodiments, the PEG or the conjugated lipid can include 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can include 30-70% ionizable lipid by mole or by total weight of the composition, 0-60% cholesterol by mole or by total weight of the composition, 0-30% non-cationic lipid by mole or by total weight of the composition and 1-10% conjugated lipid by mole or by total weight of the composition. Preferably, the composition includes 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example including 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation includes ionizable lipid, phospholipid, cholesterol and a PEG-ylated lipid in a molar ratio of 50:10:38.5:1.5. In some other embodiments, the lipid particle formulation includes ionizable lipid, cholesterol and a PEG-ylated lipid in a molar ratio of 60:38.5:1.5.

In some embodiments, the lipid particle includes ionizable lipid, non-cationic lipid (e.g., phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle includes ionizable lipid/non-cationic-lipid/sterol/conjugated lipid at a molar ratio of 50:10:38.5:1.5.

In an aspect, the disclosure provides a lipid nanoparticle formulation including phospholipids, lecithin, phosphatidylcholine and phosphatidylethanolamine.

In some embodiments, one or more additional compounds can also be included. Those compounds can be administered separately, or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, the LNPs include biodegradable, ionizable lipids. In some embodiments, the LNPs include (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about I mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

An LNP may, in some instances, be relatively homogenous. A polydispersity index may be used to indicate the homogeneity of an LNP, e.g., the particle size distribution of the lipid nanoparticles. A small (e.g., less than 0.3) polydispersity index generally indicates a narrow particle size distribution. An LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of an LNP may be from about 0.10 to about 0.20.

The zeta potential of an LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of an LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid, describes the amount of protein and/or nucleic acid that is encapsulated or otherwise associated with an LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

An LNP may optionally include one or more coatings. In some embodiments, an LNP may be formulated in a capsule, film, or tablet having a coating. A capsule, film, or tablet including a composition described herein may have any useful size, tensile strength, hardness, or density.

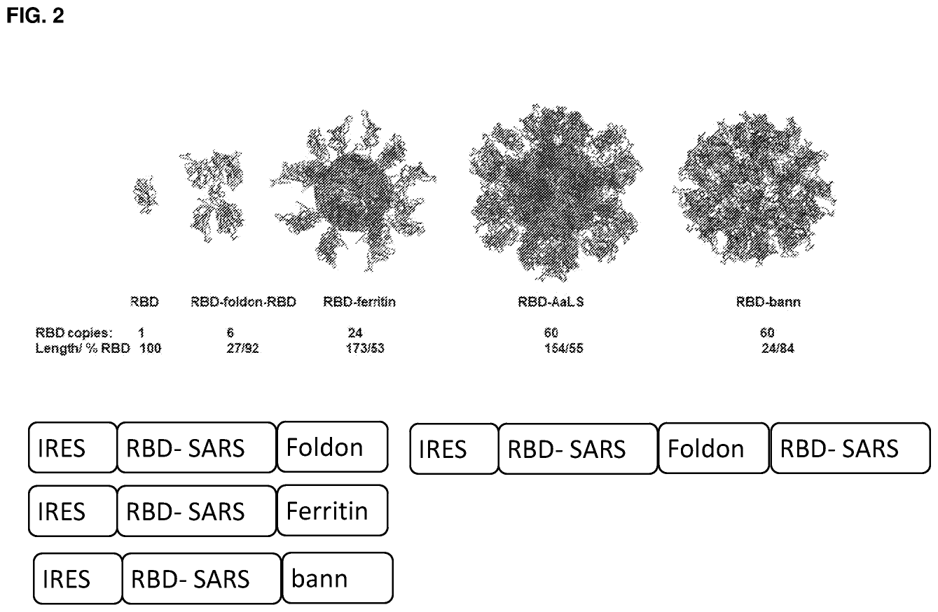

Additional exemplary lipids, formulations, methods, and characterization of LNPs are taught by WO2020/061457, WO2021/113777, and WO2021226597, each of which is incorporated herein by reference in its entirety. Further exemplary lipids, formulations, methods, and characterization of LNPs are taught by Hou et al. Lipid nanoparticles for mRNA delivery. Nat Rev Mater (2021). doi.org/10.1038/s41578-021-00358-0, which is incorporated herein by reference in its entirety (see, for example, exemplary lipids and lipid derivatives of FIG. 2 of Hou et al.).

In some embodiments, in vitro or ex vivo cell lipofections are performed using Lipofectamine MessengerMax (Thermo Fisher) or TranslT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. ANGEW CHEM INT ED ENGL 51(34):8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference, and are useful for delivery of circular polyribonucleotides and linear polyribonucleotides described herein.

Additional specific LNP formulations useful for delivery of nucleic acids (e.g., circular polyribonucleotides, linear polyribonucleotides) are described in U.S. Pat. Nos. 8,158,601 and 8,168,775, both incorporated by reference, which include formulations used in patisiran, sold under the name ONPATTRO.

In embodiments, a polyribonucleotide (e.g., a circular polyribonucleotide, a linear polyribonucleotide) encoding at least a portion (e.g., an antigenic portion) of an immunogen or polypeptide described herein is formulated in an LNP, wherein: (a) the LNPs comprise a cationic lipid, a neutral lipid, a cholesterol, and a PEG lipid, (b) the LNPs have a mean particle size of between 80 nm and 160 nm, and (c) the polyribonucleotide comprises: (i) a 5′-cap structure; (ii) a 5′-UTR; (iii) N1-methyl-pseudouridine, cytosine, adenine, and guanine; (iv) a 3′-UTR; and (v) a poly-A region. In embodiments, the polyribonucleotide (e.g., circular polyribonucleotide, linear polyribonucleotide) formulated in an LNP is a vaccine.

Exemplary dosing of polyribonucleotide (e.g., a circular polyribonucleotide, a linear polyribonucleotide) LNP may include about 0.1, 0.25, 0.3, 0.5, 1, 2, 3, 4, 5, 6, 8, 10, or 100 mg/kg (RNA).

In some embodiments, a dose of a polyribonucleotide (e.g., a circular polyribonucleotide, a linear polyribonucleotide) immunogenic composition described herein is between 30-200 mcg, e.g., 30 mcg, 50 mcg, 75 mcg, 100 mcg, 150 mcg, or 200 mcg. Exemplary dosing of AAV including a polyribonucleotide (e.g., a circular polyribonucleotide, a linear polyribonucleotide) may include an MOI of about 10¹¹, 10¹², 10¹³, and 10¹⁴ vg/kg.

### Therapeutics Methods

The disclosure provides compositions and methods that are useful as treatments or prophylactics, for example, compositions and methods that comprise antibodies that can be used to protect a subject (e.g., the subject for immunization or the subject for treatment) against the effects of a coronavirus infection. For example, a circular polyribonucleotide of the disclosure can be administered to a subject (e.g., a subject for immunization) to stimulate production of antibodies (e.g., human polyclonal antibodies) that bind to desired coronavirus immunogens/and or epitopes. A linear polyribonucleotide of the disclosure can be administered to a subject (e.g., a subject for immunization) to stimulate production of antibodies (e.g., human polyclonal antibodies) that bind to desired coronavirus immunogens/and or epitopes. The antibodies can be obtained from the subject (e.g., after immunization of the subject for immunization) and formulated for administration to a subject (e.g., a subject for treatment, such as a human subject for treatment), for example, as a treatment or prophylactic. The antibodies can provide protection against, for example, a coronavirus that expresses the immunogens and/or epitopes. In another example, a circular polyribonucleotide can be administered to a human subject (e.g., a human subject for immunization) to stimulate production of antibodies in the human subject that bind to desired immunogens/and or epitopes. In another example, a linear polyribonucleotide can be administered to a human subject (e.g., a human subject for immunization) to stimulate production of antibodies in the human subject that bind to desired immunogens/and or epitopes. In some embodiments, the disclosure provides compositions for use in treating or prophylaxis of a coronavirus infection.

Non-limiting examples of conditions and diseases that can be treated by compositions and methods of the disclosure include those caused by or associated with a coronavirus disclosed herein, for example coronavirus infections. In some embodiments, a condition is caused by or associated with a SARS-CoV. In some embodiments, a condition is caused by or associated with SARS-CoV-2. In some embodiments, a condition is coronavirus disease of 2019 (COVID-19). In some embodiments, a condition is caused by or associated with MERS-CoV.

In some embodiments, the polyclonal antibodies are produced by immunizing a non-human animal or human subject (e.g., a non-human animal or human subject for immunization) with a circular polyribonucleotide of the disclosure, plasma are collected from the non-human animal or human subject (e.g., after immunization of the non-human animal or human subject for immunization), and polyclonal antibodies are purified from the plasma. In some embodiments, the polyclonal antibodies are produced by immunizing a non-human animal or human subject (e.g., a non-human animal or human subject for immunization) with a linear polyribonucleotide of the disclosure, plasma are collected from the non-human animal or human subject (e.g., after immunization of the non-human animal or human subject for immunization), and polyclonal antibodies are purified from the plasma. Optionally, purified polyclonal antibodies from more than one non-human animal or human subject (e.g., after immunization of the more than one non-human animal or human subject for immunization), multiple purified polyclonal antibody samples from the same non-human animal or human subject (e.g., after immunization of the non-human animal or human subject for immunization), or a combination thereof, are pooled together and administered to a subject (e.g., a subject for treatment) in need thereof, e.g., a human subject (e.g., a human subject for treatment) in need thereof. In some embodiments, the polyclonal antibodies are formulated in a polyclonal antibody preparation, e.g., a polyclonal antibody preparation against a coronavirus. A method of producing a human polyclonal antibody preparation against a coronavirus comprising (a) administering to an animal (e.g., an animal for immunization) capable of producing antibodies an immunogenic composition comprising a polyribonucleotide (e.g., a circular polyribonucleotide or a linear polyribonucleotide) that comprises a sequence encoding a coronavirus immunogen, (b) collecting blood or plasma from the mammal, (c) purifying polyclonal antibodies against the coronavirus from the blood or plasma, and (d) formulating polyclonal antibodies as a therapeutic or pharmaceutical preparation for human use (e.g., administration to a human subject for treatment) or a veterinarian preparation for non-human animal use (e.g., administration to a non-human animal subject for treatment).

In some embodiments, the method further comprises monitoring the subject (e.g., the subject for treatment) having a coronavirus infection, the subject (e.g., the subject for treatment) at risk for exposure to a coronavirus infection, or the subject (e.g., the subject for treatment) in need thereof for the presence of the polyclonal antibodies for the coronavirus immunogen. In some embodiments, the monitoring is prior to administration of the polyclonal antibodies and/or after the administration of the polyclonal antibodies.

In practicing the methods of treatment or use provided herein, therapeutically effective amounts of the compounds (e.g., agents, such as a circular polyribonucleotide or antibody) described herein are administered in pharmaceutical compositions to a subject (e.g., the subject for immunization or the subject for treatment) having a disease or condition to be treated or requiring prophylaxis. In practicing the methods of treatment or use provided herein, therapeutically effective amounts of the compounds (e.g., agents, such as a linear polyribonucleotide or antibody) described herein are administered in pharmaceutical compositions to a subject (e.g., the subject for immunization or the subject for treatment) having a disease or condition to be treated or requiring prophylaxis. In some embodiments, the subject (e.g., the subject for immunization or the subject for treatment) is a mammal such as a human. A therapeutically effective amount can vary widely depending on the severity of the disease, the age and relative health of the subject (e.g., the subject for immunization or the subject for treatment), the potency of the compounds used, characteristics of a given coronavirus, and other factors.

### Methods and Routes of Administering

A composition (e.g., a pharmaceutical composition) disclosed herein can be administered in a therapeutically effective amount by various forms and routes including, for example, oral, or topical administration. In some embodiments, a composition can be administered by parenteral, intravenous, subcutaneous, intramuscular, intradermal, intraperitoneal, intracerebral, subarachnoid, intraocular, intrasternal, ophthalmic, endothelial, local, intranasal, intrapulmonary, rectal, intraarterial, intrathecal, inhalation, intralesional, intradermal, epidural, intracapsular, subcapsular, intracardiac, transtracheal, subcuticular, subarachnoid, or intraspinal administration, e.g., injection or infusion. In some embodiments, a composition can be administered by absorption through epithelial or mucocutaneous linings (e.g., oral mucosa, rectal and intestinal mucosa administration). In some embodiments, the composition is delivered via multiple administration routes.

In some embodiments, the composition is administered by intravenous infusion. In some embodiments, the composition is administered by slow continuous infusion over a long period, such as more than 24 hours. In some embodiments, the composition is administered as an intravenous injection or a short infusion.

A pharmaceutical composition can be administered in a local manner, for example, via injection of the agent directly into an organ, optionally in a depot or sustained release formulation or implant. A pharmaceutical composition can be provided in the form of a rapid release formulation, in the form of an extended-release formulation, or in the form of an intermediate release formulation. A rapid release form can provide an immediate release. An extended-release formulation can provide a controlled release or a sustained delayed release. In some embodiments, a pump can be used for delivery of the pharmaceutical composition. In some embodiments, a pen delivery device can be used, for example, for subcutaneous delivery of a composition of the disclosure.

A pharmaceutical composition provided herein can be administered in conjunction with other therapies, for example, an antiviral therapy, an antibiotic, a cell therapy, a cytokine therapy, or an anti-inflammatory agent. In some embodiments, a circular polyribonucleotide or antibody described herein can be used singly or in combination with one or more therapeutic agents as a component of mixtures. In some embodiments, a linear polyribonucleotide or antibody described herein can be used singly or in combination with one or more therapeutic agents as a component of mixtures.

### Doses and Frequency

Therapeutic agents described herein can be administered before, during, or after the occurrence of a disease or condition, and the timing of administering the composition containing a therapeutic agent can vary. In some cases, the compositions can be used as a prophylactic and can be administered continuously to subjects (e.g., the subject for immunization or the subject for treatment) with a susceptibility to a coronavirus or a propensity to a condition or disease associated with a coronavirus. Prophylactic administration can lessen the likelihood of the occurrence of the infection, disease, or condition, or can reduce the severity of the infection, disease or condition.

The compositions can be administered to a subject (e.g., the subject for immunization or the subject for treatment) after (e.g., as soon as possible after) the onset of the symptoms. The compositions can be administered to a subject (e.g., the subject for immunization or the subject for treatment) after (e.g., as soon as possible after) a test result, for example, a test result that provides a diagnosis, a test that shows the presence of a coronavirus in a subject (e.g., the subject for immunization or the subject for treatment), or a test showing progress of a condition, e.g., a decreased blood oxygen levels. A therapeutic agent can be administered after (e.g., as soon as is practicable after) the onset of a disease or condition is detected or suspected. A therapeutic agent can be administered after (e.g., as soon as is practicable after) a potential exposure to a coronavirus, for example, after a subject (e.g., the subject for immunization or the subject for treatment) has contact with an infected subject or learns they had contact with an infected subject that may be contagious.

A circular polyribonucleotide, antibody, or therapeutic agent described herein are administered at any interval desired. A linear polyribonucleotide, antibody, or therapeutic agent described herein are administered at any interval desired.

Actual dosage levels of an agent of the disclosure (e.g., circular polyribonucleotide, linear polyribonucleotide, antibody, or therapeutic agent) may be varied so as to obtain an amount of the agent to achieve the desired therapeutic response for a particular subject, composition, and mode of administration, without being toxic to the subject (e.g., the subject for immunization or the subject for treatment). The selected dosage level can depend upon a variety of pharmacokinetic factors including the activity of the particular compositions of the present invention employed, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts.

Dosage regimens may be adjusted to provide the optimum desired response (e.g., a therapeutic and/or prophylactic response). For example, a single bolus may be administered, several divided doses may be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects (e.g., the subjects for immunization or the subjects for treatment); each unit contains a predetermined quantity of active agent calculated to produce the desired therapeutic effect in association with the required pharmaceutical carrier. The specification for the dosage unit forms of the disclosure can be determined by and directly dependent on (a) the unique characteristics of the active agent and the particular therapeutic effect to be achieved, and (b) the limitations inherent in the art of compounding such an active agent for the treatment of sensitivity in individuals. A dose can be determined by reference to a plasma concentration or a local concentration of the circular polyribonucleotide or antibody. A dose can be determined by reference to a plasma concentration or a local concentration of the linear polyribonucleotide or antibody.

A pharmaceutical composition described herein can be in a unit dosage form suitable for a single administration of a precise dosage. In unit dosage form, the formulation can be divided into unit doses containing appropriate quantities of one or more circular polyribonucleotides, antibodies, and/or therapeutic agents. In unit dosage form, the formulation can be divided into unit doses containing appropriate quantities of one or more linear polyribonucleotides, antibodies, and/or therapeutic agents. The unit dosage can be in the form of a package containing discrete quantities of the formulation. Non-limiting examples are packaged injectables, vials, and ampoules. An aqueous suspension composition disclosed herein can be packaged in a single-dose non-reclosable container. Multiple-dose reclosable containers can be used, for example, in combination with or without a preservative. A formulation for injection disclosed herein can be present in a unit dosage form, for example, in ampoules, or in multi dose containers with a preservative.

A dose can be based on the amount of the agent per kilogram of body weight of a subject (e.g., the subject for immunization or the subject for treatment). A dose of an agent (e.g., antibody) is in the range of 10-3000 mg/kg, e.g., 100-2000 mg/kg, e.g., 300-500 mg/kg/day for 1-10 or 1-5 days; e.g., 400 mg/kg/day for 3-6 days; e.g., 1 g/kg/d for 2-3 days.

### Subjects

A composition is provided for use in treatment or prophylaxis of a condition disclosed herein, such as an infection with a coronavirus. The composition can be administered to a subject (e.g., the subject for immunization or the subject for treatment) that has a coronavirus infection or an associated disease or condition. The composition can be administered as a prophylactic to subjects (e.g., subjects for immunization or subjects for treatment) with a propensity for coronavirus infection or a susceptibility to an associated condition or disease in order to lessen a likelihood of the infection, disease or condition, or to reduce the severity of the infection, disease or condition.

A subject (e.g., the subject for immunization or the subject for treatment) can be a subject that is infected with a coronavirus. A subject (e.g., the subject for immunization or the subject for treatment) can be a subject that tested positive for the coronavirus. A subject (e.g., the subject for immunization or the subject for treatment) can be a subject that has been exposed to a coronavirus. A subject (e.g., the subject for immunization or the subject for treatment) can be a subject that has potentially been exposed to a coronavirus. A subject (e.g., the subject for immunization or the subject for treatment) can be a subject that is exhibiting one or more signs and/or symptoms consistent with infection with a coronavirus.

In some embodiments, a subject (e.g., the subject for immunization or the subject for treatment) is a subject that is at high risk of coming into contact with a coronavirus of the disclosure. For example, a subject (e.g., the subject for immunization or the subject for treatment) may be a health care worker, a laboratory worker, or a first responder that is more likely to come into contact with a coronavirus (e.g., SARS-CoV2) of the disclosure. A subject (e.g., the subject for immunization or the subject for treatment) may work at a health care facility, e.g., a hospital, doctor's surgery, inpatient facility, outpatient facility, urgent care facility, retirement home, aged care facility, or nursing home.

In some embodiments, a subject (e.g., the subject for immunization or the subject for treatment) is a subject that is at high risk of complications if infected with a coronavirus of the disclosure. For example, a subject (e.g., the subject for immunization or the subject for treatment) can have a co-morbidity, an age over 50, type 1 diabetes mellitus, type 2 diabetes mellitus, insulin resistance, or a combination thereof. In some embodiments, a subject is an immunocompromised subject. In some embodiments, a subject (e.g., the subject for immunization or the subject for treatment) is on immunosuppressive drugs. In some embodiments, a subject (e.g., the subject for immunization or the subject for treatment) is a transplant recipient that is on immunosuppressive drugs. In some embodiments, a subject (e.g., the subject for immunization or the subject for treatment) is undergoing therapy for cancer, e.g., chemotherapy, that may decrease the function of the immune system.

A subject (e.g., the subject for immunization or the subject for treatment) can be a mammal. A subject (e.g., the subject for immunization or the subject for treatment) can be a human. A subject (e.g., the subject for immunization or the subject for treatment) can be a non-human animal. The non-human animal can be an agricultural animal, e.g., a cow, pig, sheep, horse, or goat; a pet, e.g., a cat or dog; or a zoo animal, e.g., a feline.

### Kits

In some aspects, the disclosure provides a kit. In some embodiments, the kit includes (a) a circular polyribonucleotide, an immunogenic composition, or a pharmaceutical composition described herein, and, optionally (b) informational material. In some embodiments, the kit further comprises an adjuvant described herein, which may be provided in a separate composition to be administered in combination with the circular polyribonucleotide, an immunogenic composition, or a pharmaceutical composition as part of a defined dosing regimen. The informational material may be descriptive, instructional, marketing, or other material that relates to the methods described herein and/or the use of the pharmaceutical composition or circular polyribonucleotide for the methods described herein. The pharmaceutical composition or circular polyribonucleotide may comprise material for a single administration (e.g., single dosage form), or may comprise material for multiple administrations (e.g., a “multidose” kit).

The informational material of the kits is not limited in its form. In one embodiment, the informational material may include information about production of the pharmaceutical composition, the pharmaceutical drug substance, or the pharmaceutical drug product, molecular weight of the pharmaceutical composition, the pharmaceutical drug substance, or the pharmaceutical drug product, concentration, date of expiration, batch, or production site information, and so forth. In one embodiment, the informational material relates to methods for administering a dosage form of the pharmaceutical composition. In one embodiment, the informational material relates to methods for administering a dosage form of the circular polyribonucleotide.

In addition to a dosage form of the pharmaceutical composition and circular polyribonucleotide described herein, the kit may include other ingredients, such as a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, and/or a second agent for treating a condition or disorder described herein. Alternatively, the other ingredients may be included in the kit, but in different compositions or containers than a pharmaceutical composition or circular polyribonucleotide described herein. In such embodiments, the kit may include instructions for admixing a pharmaceutical composition or nucleic acid molecule (e.g., a circular polyribonucleotide) described herein and the other ingredients, or for using a pharmaceutical composition or nucleic acid molecule (e.g., a circular polyribonucleotide) described herein together with the other ingredients.

In some embodiments, the components of the kit are stored under inert conditions (e.g., under Nitrogen or another inert gas such as Argon). In some embodiments, the components of the kit are stored under anhydrous conditions (e.g., with a desiccant). In some embodiments, the components are stored in a light blocking container such as an amber vial.

A dosage form of a pharmaceutical composition or nucleic acid molecule (e.g., a circular polyribonucleotide) described herein may be provided in any form, e.g., liquid, dried or lyophilized form. It is preferred that a pharmaceutical composition or nucleic acid molecule (e.g., a circular polyribonucleotide) described herein be substantially pure and/or sterile. When a pharmaceutical composition or nucleic acid molecule (e.g., a circular polyribonucleotide) described herein is provided in a liquid solution, the liquid solution preferably is an aqueous solution, with a sterile aqueous solution being preferred. When a pharmaceutical composition or nucleic acid molecule (e.g., a circular polyribonucleotide) described herein is provided as a dried form, reconstitution generally is by the addition of a suitable solvent. The solvent, e.g., sterile water or buffer, can optionally be provided in the kit.

The kit may include one or more containers for the composition containing a dosage form described herein. In some embodiments, the kit contains separate containers, dividers or compartments for the composition and informational material. For example, the pharmaceutical composition or circular polyribonucleotide may be contained in a bottle, vial, or syringe, and the informational material may be contained in a plastic sleeve or packet. In other embodiments, the separate elements of the kit are contained within a single, undivided container. For example, the dosage form of a pharmaceutical composition or nucleic acid molecule (e.g., a circular polyribonucleotide) described herein is contained in a bottle, vial or syringe that has attached thereto the informational material in the form of a label. In some embodiments, the kit includes a plurality (e.g., a pack) of individual containers, each containing one or more unit dosage forms of a pharmaceutical composition or circular polyribonucleotide described herein. For example, the kit includes a plurality of syringes, ampules, foil packets, or blister packs, each containing a single unit dose of a dosage form described herein.

The containers of the kits can be airtight, waterproof (e.g., impermeable to changes in moisture or evaporation), and/or light tight.

The kit optionally includes a device suitable for use of the dosage form, e.g., a syringe, pipette, forceps, measured spoon, swab (e.g., a cotton swab or wooden swab), or any such device.

The kits of the invention may include dosage forms of varying strengths to provide a subject with doses suitable for one or more of the initiation phase regimens, induction phase regimens, or maintenance phase regimens described herein. Alternatively, the kit may include a scored tablet to allow the user to administered divided doses, as needed.

### EXAMPLES

The following examples are included for illustrative purposes only and are not intended to limit the scope of the invention.

### Example 1: Circular RNA Constructs

This example describes design of novel SARS-CoV-2 open reading frames (ORFs) and circRNA constructs.

In this Example, SARS-CoV-2 ORFs and circular RNA constructs encoding SARS-CoV-2 ORFs were designed as described in TABLE 2.

### Example 2: In Vitro Production of Circular RNAs Encoding SARS-CoV-2 Immunogens

This example demonstrates in vitro production of circular RNAs.

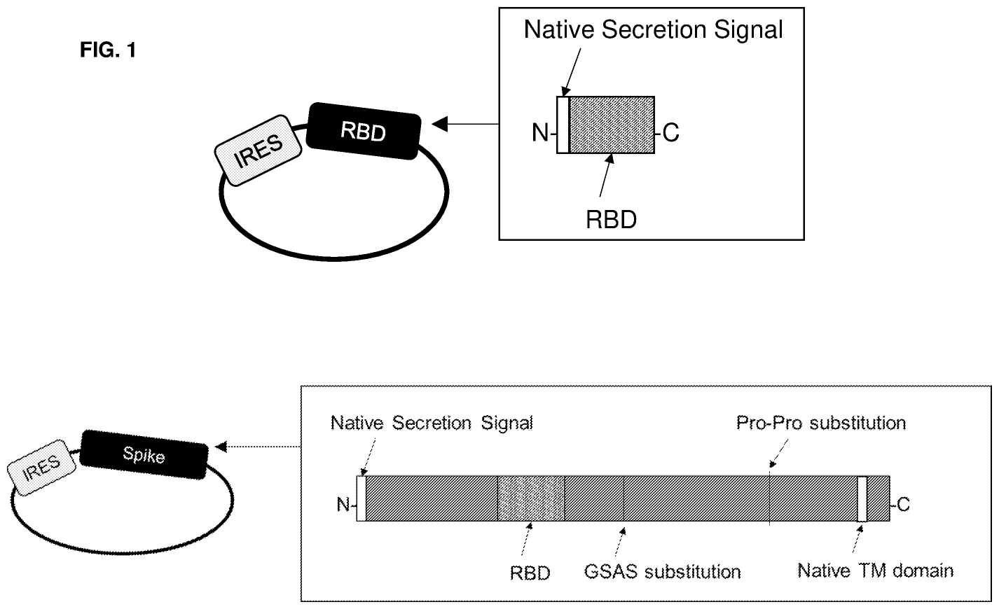

Circular RNAs were designed to include an IRES, an ORF encoding a modified SARS-CoV-2 spike immunogen or RBD immunogen (as described in Example 1), and two spacer elements flanking the IRES-ORF. Circularization enables rolling circle translation, multiple ORFs with alternating stagger elements for discrete ORF expression and controlled protein stoichiometry, and an IRES that targets RNA for ribosomal entry. Exemplary drawings of circular polyribonucleotide comprising a sequence encoding a coronavirus immunogen is shown in FIGS. 1 and 3-5.

In this Example, circular RNAs were generated as follows. Unmodified linear RNA was synthesized by in vitro transcription using T7 RNA polymerase from a DNA segment. Transcribed RNA was purified with an RNA purification system (New England Biolabs, Inc.), treated with RNA 5′phosphohydrolase (RppH) (New England Biolabs, M0356) following the manufacturer's instructions, and purified again with the RNA purification system. RppH-treated linear RNA was circularized using a splint DNA. Alternately or in addition to treatment with 5′RppH, the RNA was transcribed under conditions with excess GMP over GTP.

Splint-ligation was performed as follows: circular RNA was generated by treatment of the transcribed linear RNA and a DNA splint (5′-GTTTTTCGGCTATTCCCAATAGCCGTTTTG-3′) (SEQ ID NO: 47) using T4 DNA ligase 1 (New England Bio, Inc., M0437M). To purify the circular RNAs, ligation mixtures were resolved on 4% denaturing PAGE and RNA corresponding to each of the circular RNAs were excised. Excised RNA gel fragments were crushed, and RNA was eluted with gel elution buffer (0.5 M Sodium Acetate, 0.1% SDS, 1 mM EDTA) for one hour at 37° C. The eluted buffer was harvested, and RNA was eluted again by adding gel elution buffer to the crushed gel and incubated for one hour. Gel debris was removed by centrifuge filters and RNA was precipitated with ethanol. Agarose gel electrophoresis was used as a quality control measurement for validating purity and circularization. Additionally, or alternatively, the circular RNAs were purified using column chromatography.

### Example 3: MRNA Constructs

This example describes design of novel mRNA constructs encoding SARS-CoV-2 ORFs.

In this Example, linear RNA constructs encoding SARS-CoV-2 ORFs were designed as described in Table 6.

### Example 4: In Vitro Production of mRNAs Encoding SARS-CoV-2 Immunogens

This example demonstrates in vitro production of mRNAs.

In this Example, mRNA was designed with an ORF encoding a modified SARS-CoV-2 spike immunogen or RBD as described in Example 3.

In this Example, modified mRNA was made by in vitro transcription. RNA was fully substituted with Pseudo-Uridine and 5-Methyl-C, capped with CleanCap™ AG, included 5′ and 3′ human alpha-globin UTRs, and was polyadenylated. mRNA was Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000). Agarose gel electrophoresis was used as a quality control measurement for validating purity and circularization.

### Example 5: Expression of Non-Secreted SARS-CoV-2 Immunogen from RNA in Mammalian Cells

In this Example, circular RNA or mRNA encoding SARS-CoV-2 spike immunogens were designed and produced and purified by the methods described herein. Circular RNAs and mRNAs are formulated in MessengerMax and 0.1 picomoles of circular RNA is transfected into HEK293 cells (10 000 cells per well) according to the manufacturer's instructions.

Spike immunogen expression is measured using a SARS-CoV-2 spike immunogen-specific ELISA at 24, 48, and 72 hours. To measure expression, cells are lysed in each well at the appropriate timepoint, using a lysis buffer and a protease inhibitor. The cell lysate is retrieved and centrifuged at 12,000 rpm for 10 minutes. Supernatant is collected. In this example, a SARS-CoV-2 2019 spike immunogen detection sandwich ELISA kit is used (SARS-CoV-2 (2019-nCoV) Spike Detection ELISA Kit, Sino Biological, KIT40591) according to the manufacturer's instructions.

### Example 6: Administration of RNA Encoding SARS-CoV-2 Immunogens to a Human Subject

This example describes the administration of a circular RNA encoding a SARS-CoV-2 immunogen to a human subject.

In this Example, circular RNA or mRNA encoding SARS-CoV-2 immunogens were designed and produced and purified by the methods described herein.

In this example, in one approach, RNA is formulated (with a lipid carrier (e.g., TransiT), formulated with a cationic polymer (e.g., protamine), formulated with a lipid nanoparticle, or unformulated), to obtain a first set of circular RNA preparations or a first set of linear RNA preparations. In a second approach, Addavax™ adjuvant (Invivogen), MF59® adjuvant, or complete Freund's adjuvant, is formulated with the RNA-lipid carrier mixture or the unformulated RNA preparation (e.g., circular RNA preparation or linear RNA preparation), as described in Beigel J H et al. (Lancet Infect. Dis., 18: 410-418 (2018)), to obtain a second set of circular RNA preparations or a second set of linear RNA preparations. Circular RNA or linear RNA is formulated to obtain the circular RNA preparations or linear RNA preparations shortly before injection into the human subject.

In this example, a human subject is immunized with the circular RNA preparations (i.e., a first circular RNA preparation or a second circular RNA preparation), linear RNA preparations (i.e., a first circular RNA preparation or a second circular RNA preparation) via intramuscular or intradermal injection. The circular RNA preparations or linear RNA preparations are administered to the human subject at least one time, at least two times, at least 3 times, or more to elicit an immunogenic response in the human subject.

### Example 7: Expression of Multiple Immunogens from Circular RNAs in Mammalian Cells

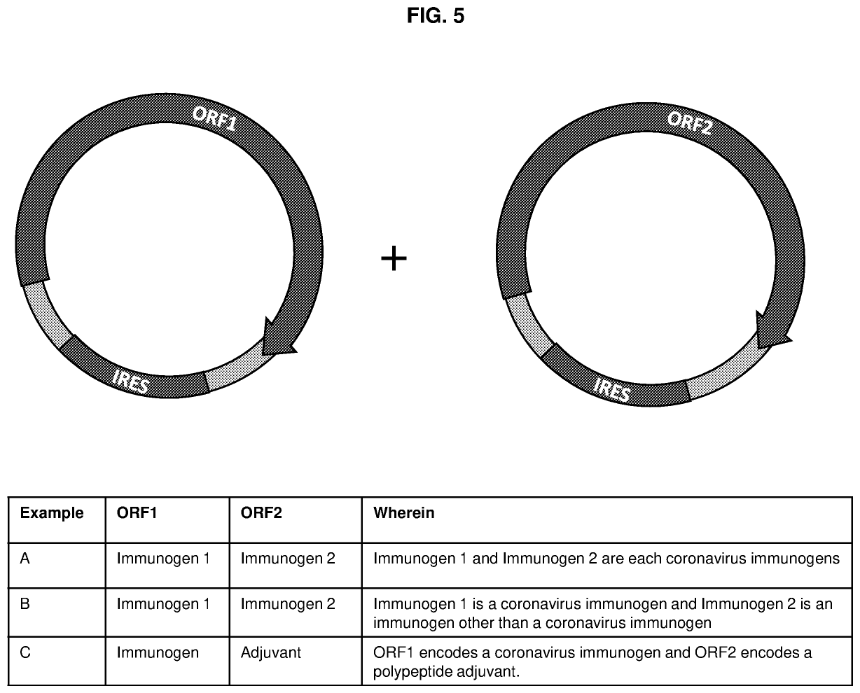

This example demonstrates expression of multiple immunogens from circular RNAs in mammalian cells. An exemplary schematic of these constructs is shown in FIG. 5.

### Experiment 1

A first circular RNA encoding a SARS-CoV-2 RBD immunogen (Nucleic acid SEQ ID NO: 56; Amino acid SEQ ID NO: 55) was designed and produced and purified by the methods described herein. A second circular RNA encoding a SARS-CoV-2 Spike immunogen (Nucleic acid SEQ ID NO: 54; Amino acid SEQ ID NO. 53) was designed and produced and purified by the methods described herein. The first circular RNA and the second circular RNA were mixed together to obtain a mixture. The mixture (1 picomoles of each of the circular RNAs) was transfected into HeLa cells (100,000 cells per well in a 24 well plate) using Lipofectamine MessengerMax (ThermoFisher, LMRNA015). As controls, the first circular RNA and the second circular RNA were also separately transfected into HeLa cells using MessengerMax.

RBD immunogen expression was measured at 24 hours using a SARS-CoV-2 RBD immunogen-specific ELISA. Spike immunogen expression was measured at 24 hours by flow cytometry.

From the transfection with the mixture, SARS-Co-V-2 RBD immunogen was detected in the HeLa cell supernatant and SARS-CoV-2 Spike immunogen was detected on the cell surface of the HeLa cells. From the transfection with the first circular RNA, SARS-CoV-2 RBD immunogen was detected, but SARS-CoV-2 Spike immunogen was not detected. From the transfection with the second circular RNA, SARS-CoV-2 Spike immunogen was detected, but SARS-CoV-2 RBD immunogen was not detected. This demonstrates that both SAR-CoV-2 RBD and SARS-CoV-2 Spike immunogens were expressed in mammalian cells from a combination mixture of circular RNAs.

### Experiment 2

A first circular RNA encoding a SARS-CoV-2 RBD immunogen (Nucleic acid SEQ ID NO: 56; Amino acid SEQ ID NO. 55) was designed and produced and purified by the methods described herein. A second circular RNA was designed with an IRES and ORF encoding Gaussia Luciferase (GLuc) (Nucleic acid SEQ ID NO: 58; Amino acid SEQ ID NO. 57) and produced and purified as described in Example 2. The first circular RNA and the second circular RNA were separately complexed with Lipofectamine MessengerMax (ThermoFisher, LMRNA015), and then mixed together to obtain a mixture. The mixture (0.1 picomoles of each circular RNAs) was transfected into HeLa cells (20,000 cells per well in a 96 well plate). As controls, the first circular RNA and the second circular RNA were also separately transfected into HeLa cells using MessengerMax.

RBD immunogen expression was measured at 24 hours using a SARS-CoV-2 RBD immunogen-specific ELISA. GLuc activity was measured at 24 hours using a Gaussia Luciferase activity assay (Thermo Scientific Pierce).

From the transfection with the mixture, SARS-CoV-2 RBD immunogen and GLuc activity were detected in the HeLa cell supernatant at 24 hrs. From the transfection with the first circular RNA, SARS-CoV-2 RBD immunogen was detected, but GLuc activity was not detected. From the transfection with the second circular RNA, GLuc activity was detected, but SARS-CoV-2 RBD immunogen was not detected.

This demonstrates that both SAR-CoV-2 RBD and GLuc immunogens were expressed in mammalian cells from a combination mixture of circular RNAs.

### Experiment 3

A first circular RNA encoding a SARS-CoV-2 RBD immunogen (Nucleic acid SEQ ID NO: 56; Amino acid SEQ ID NO. 55) was designed and produced and purified by the methods described herein. A second circular RNA was designed to include an IRES followed by an ORF encoding hemagglutinin (HA) from Influenza A H1N1, A/California/07/2009 (Nucleic acid SEQ ID NO: 60; Amino acid SEQ ID NO: 59) and produced and purified as described in Example 2. The first circular RNA and the second circular RNA were mixed together to obtain a mixture. The mixture (1 picomoles of each circular RNA) was transfected into HeLa cells (100,000 cells per well in a 24 well plate) using Lipofectamine MessengerMax (ThermoFisher, LMRNA015). As controls, the first circular RNA and the second circular RNA were also separately transfected into HeLa cells using MessengerMax.

RBD immunogen expression was measured at 24 hours using a SARS-CoV-2 RBD immunogen-specific ELISA. HA immunogen expression was measured at 24 hours using immunoblot. Briefly, for immunoblot, 24 hours after transfection, cells were lysed and Western blot was performed to detect the HA immunogen using Influenza A H1N1 HA (A/California/07/2009) monoclonal antibody (MA5-29920 (Thermo Fisher)) as the primary antibody and goat anti-mouse IgG H&L (HRP) as the secondary antibody (Abcam, ab 97023). For loading control alpha tubulin was used with alpha tubulin (DM1A) mouse antibody as the primary antibody (Cell Signaling Technology, CST #3873) and goat anti-mouse IgG H&L (HRP) as the secondary antibody (Abcam, ab 97023).

From the transfection with the mixture, both SARS-CoV-2 RBD and Influenza HA immunogens were detected. From the transfection with the first circular RNA, SARS-CoV-2 RBD was detected, but Influenza HA immunogen was not detected. From the transfection with the second circular RNA, Influenza HA immunogen was detected, but SARS-CoV-2 RBD immunogen was not detected. This demonstrates that both SAR-CoV-2 RBD and Influenza HA immunogens were expressed in mammalian cells from a combination mixture of circular RNAs.

### Experiment 4

A first circular RNA encoding a SARS-CoV-2 Spike immunogen (Nucleic acid SEQ ID NO: 45; Amino acid SEQ ID NO: 53) was designed and produced and purified by the methods described herein. A second circular RNA was designed to include an IRES followed by an ORF encoding HA from Influenza A H1N1, A/California/07/2009 (Nucleic acid SEQ ID NO: 60; Amino acid SEQ ID NO: 59) and produced and purified as described in Example 2. The first circular RNA and the second circular RNA were mixed together to obtain a mixture. The mixture (1 picomoles of each circular RNAs) was transfected into HeLa cells (100,000 cells per well in a 24 well plate) using Lipofectamine MessengerMax (ThermoFisher, LMRNA015). As controls, the first circular RNA and the second circular RNA were also separately transfected into HeLa cells using MessengerMax.

Spike immunogen expression was measured at 24 hours by flow cytometry. HA immunogen expression was measured at 24 hours by immunoblot as described above in Experiment 3.

From the transfection with the mixture, both SARS-CoV-2 Spike immunogen and Influenza HA immunogen were detected. From the transfection with the first circular RNA, SARS-CoV-2 Spike immunogen was detected, but Influenza HA immunogen was not detected. From the transfection with the second circular RNA, Influenza HA immunogen was detected, but SARS-CoV-2 Spike immunogen was not detected. This demonstrates that both SAR-CoV-2 Spike and Influenza HA immunogens were expressed in mammalian cells from a combination mixture of circular RNAs.

This Example shows that multiple immunogens were expressed in mammalian cells from circular RNA preparations comprising different combinations of circular RNAs.

### Example 8: Multi-Immunogen Expression from Circular RNA

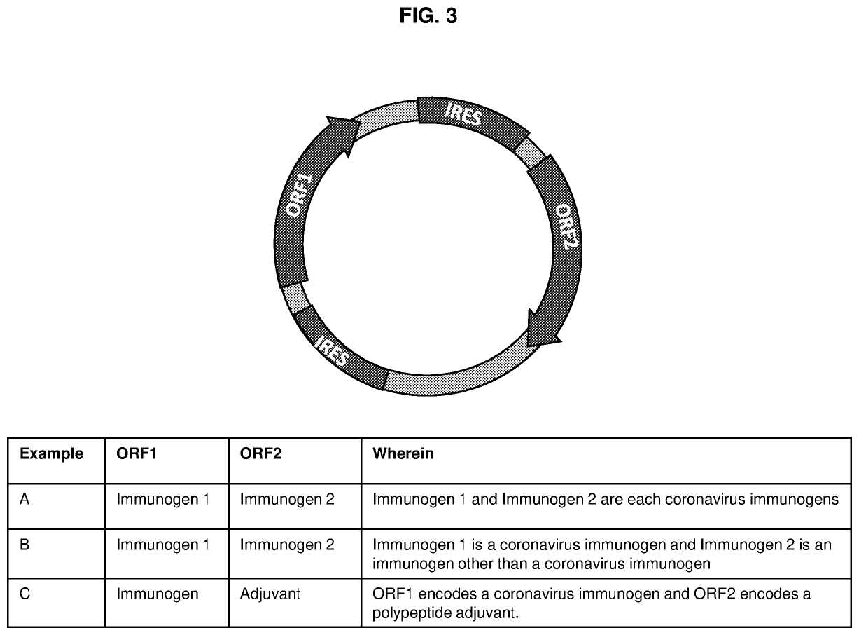

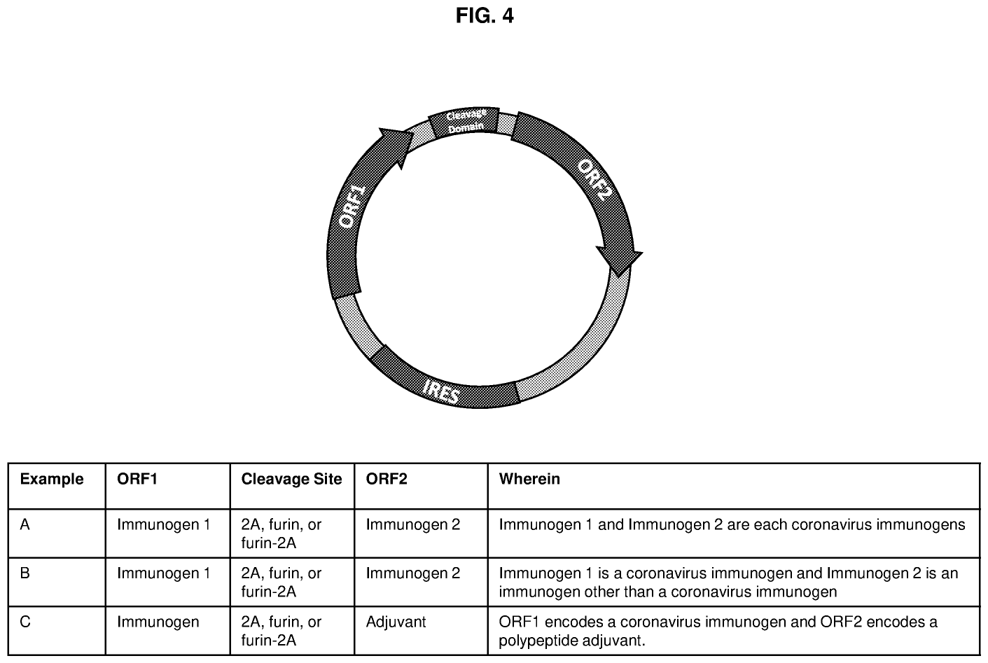

This example demonstrates expression of multiple immunogens from a circular RNA in mammalian cells. Exemplary schematics of these constructs are shown in FIGS. 3 and 4.

### Experiment 1

In this Example, a circular RNA was designed to include an IRES followed by an ORF encoding a GLuc polypeptide, a stop codon, a spacer, an IRES, an ORF encoding a SARS-CoV-2 RBD immunogen, and a stop codon. The circular RNA was produced and purified as described in Example 2. As controls, the following circular RNAs were produced as described above: (i) a circular RNA with an IRES and ORF encoding a SARS-CoV-2 RBD immunogen; (ii) a circular RNA with an IRES and ORF encoding GLuc.

The circular RNAs (0.1 picomoles) were transfected into HeLa cells (10,000 cells per well in a 96 well plate) using Lipofectamine MessengerMax (ThermoFisher, LMRNA015).

RBD immunogen expression was measured at 24 hours using a SARS-CoV-2 RBD immunogen-specific ELISA. GLuc activity was measured at 24 hours using a Gaussia Luciferase activity assay (Thermo Scientific Pierce).

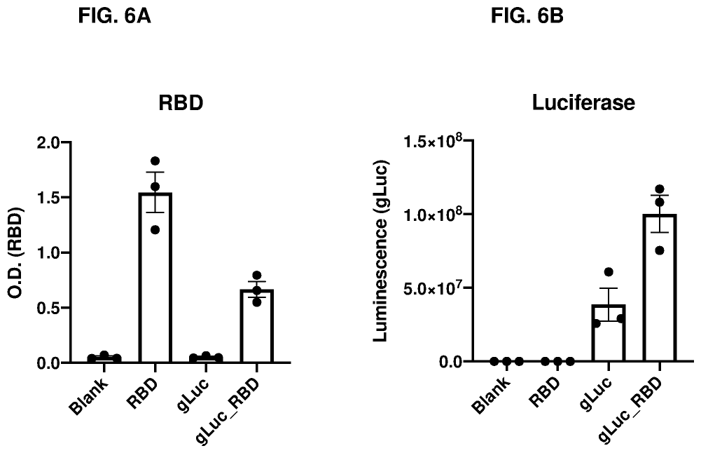

RBD immunogen expression was detected from circular RNAs encoding a SARS-CoV-2 RBD immunogen and GLuc protein (FIG. 6A). GLuc activity was detected from circular RNAs encoding GLuc polypeptide (FIG. 6B). This demonstrates that both SAR-CoV-2 RBD and GLuc immunogens were expressed in mammalian cells from a circular RNA encoding both SARS-CoV-2 RBD and GLuc immunogens.

### Experiment 2

In this Example, a circular RNA designed to include an IRES followed by an ORF encoding a SARS-CoV-2 RBD immunogen, a stop codon, a spacer, an IRES, an ORF encoding a Middle Eastern Respiratory Syndrome (MERS) RBD immunogen, and a stop codon. The circular RNA is produced and purified by the methods described herein.

The circular RNAs are transfected at various concentrations into HeLa cells (10,000 cells per well in a 96 well plate) using Lipofectamine MessengerMax (ThermoFisher, LMRNA015).

SARS-CoV-2 RBD immunogen expression is measured at 24 hours using a SARS-CoV-2 RBD immunogen-specific ELISA. MERS RBD immunogen expression is measured at 24 hours using a MERS RBD immunogen specific antibody capable of detection.

### Example 9: Immunogenicity of Multiple Immunogens from Circular RNAs in Mouse Model

This example describes expression of multiple immunogens in a subject by administrating multiple circular RNA molecules.

### Experiment 1

The immunogenicity of a circular RNA preparation comprising (a) a circular RNA encoding a SARS-CoV-2 RBD immunogen and (b) a circular RNA encoding GLuc polypeptide as a model immunogen, formulated in lipid nanoparticles, was evaluated in a mouse model. Production of antibodies to the SARS-CoV-2 RBD immunogen and GLuc activity were also evaluated in the mouse model.

A first circular RNA encoding a SARS-CoV-2 RBD immunogen (Nucleic acid SEQ ID NO: 56; Amino acid SEQ ID NO: 55) was designed and produced and purified by the methods described herein. A second circular RNA was designed with an IRES and ORF encoding GLuc polypeptide (Nucleic acid SEQ ID NO: 58; Amino acid SEQ ID NO. 57) and produced and purified by the methods described herein. The first circular RNA and the second circular RNA were mixed together to obtain a mixture. This mixture was then formulated with lipid nanoparticles to obtain a first circular RNA preparation. The first circular RNA and the second circular RNA were also separately formulated with lipid nanoparticles, and then mixed together to obtain a second circular RNA preparation.

Three mice were vaccinated intramuscularly with the first circular RNA preparation (for a total dose of 10 μg RBD+10 μg GLuc) at day 0 and with the second circular RNA preparation (for a total dose of 10 μg RBD+10 μg GLuc) at day 12. Additional mice (3 or 4 per group) were also vaccinated intramuscularly at day 0 and day 12 with: (i) a 10-μg dose of the first circular RNA formulated with lipid nanoparticles; (ii) a 10-μg dose of the second circular RNA formulated with lipid nanoparticles; or (iii) PBS.

Blood collection from each mouse was by submandibular drawing. Blood was collected into dry-anticoagulant free-tubes, at 2- and 17-days post-priming with the first circular RNA preparation. Serum was separated from whole blood by centrifugation at 1200 g for 30 minutes at 4° C. Individual serum samples were assayed for the presence of RBD-specific IgG by enzyme-linked immunosorbent assay (ELISA). ELISA plates (MaxiSorp 442404 96-well, Nunc) were coated overnight at 4° C. with SARS-CoV-2 RBD (Sino Biological, 40592-V08B; 100 ng) in 100 μL of 1× coating buffer (Biolegend, 421701). The plates were then blocked for 1 hour with blocking buffer (TBS with 2% BSA and 0.05% Tween 20). Serum dilutions (1:500, 1:1500, 1:4500, and 1:13,500) were then added to each well in 100 μL blocking buffer and incubated at room temperature for 1 hour. After washing three times with 1× Tris-buffered saline with Tween® detergent (TBS-T), plates were incubated with anti-mouse IgG HRP detection antibody (Abcam, ab97023) for 1 hour followed by three washes with TBS-T, then addition of tetramethylbenzene (Biolegend, 421101). The ELISA plate was allowed to react for 10-20 minutes and then quenched using 0.2N sulfuric acid. The optical density (O.D.) value was determined at 450 nm.

The optical density of each serum sample was divided by that of the background (plates coated with RBD, incubated only with secondary antibody). The fold over background of each sample was plotted.

The activity of GLuc was tested using a Gaussia Luciferase activity assay (Thermo Scientific Pierce). 50 μL of 1× GLuc substrate was added to 10 μL of serum to carry out the GLuc luciferase activity assay. Plates were read immediately after mixing in a luminometer instrument (Promega).

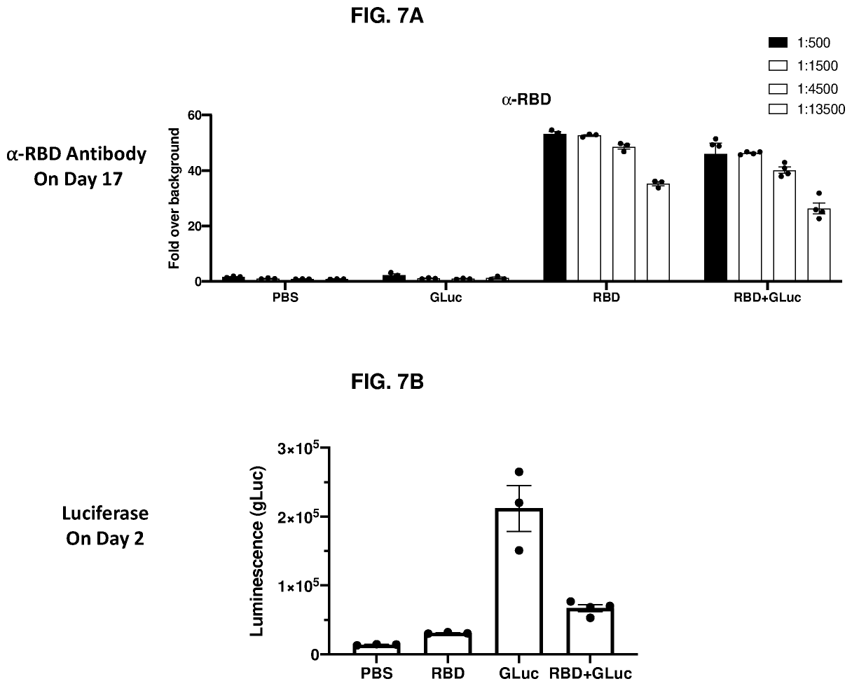

The results showed that anti-RBD antibodies were obtained at 17 days post prime (i.e., 17 days after injection with the first circular RNA preparation) (FIG. 7A) and GLuc activity was detected at 2 days post prime (i.e., 2 days after injection with the first circular RNA preparation) (FIG. 7B).

These results showed that circular RNA preparations comprising two circular RNAs encoding different immunogens induced immunogen-specific responses in mice.

### Experiment 2

The immunogenicity of a circular RNA preparation comprising (a) a circular RNA encoding a SARS-CoV-2 RBD immunogen and (b) a circular RNA encoding an Influenza hemagglutinin (HA) immunogen, formulated in lipid nanoparticles, was evaluated in a mouse model. Production of antibodies to the SARS-CoV-2 RBD and Influenza HA immunogens were also evaluated in the mouse model.

A first circular RNA encoding a SARS-CoV-2 RBD immunogen (Nucleic acid SEQ ID NO: 56; Amino acid SEQ ID NO: 55) was designed and produced and purified by the methods described herein.

A second circular RNA was designed to include an IRES followed by an ORF encoding hemagglutinin (HA) from Influenza A H1N1, A/California/07/2009 (Nucleic acid SEQ ID NO: 60; Amino acid SEQ ID NO: 59) and produced and purified by the methods described herein. The first circular RNA and the second circular RNA were mixed together to obtain a mixture. This mixture was then formulated with lipid nanoparticles as described to obtain a first circular RNA preparation. The first circular RNA and the second circular RNA were also separately formulated with lipid nanoparticles, and then mixed together to obtain a second circular RNA preparation.

Three mice were vaccinated intramuscularly with the first circular RNA preparation (for a total dose of 10 μg RBD+10 μg HA) at day 0 with the second circular RNA preparation (for a total dose of 10 μg RBD+10 μg HA) and at day 12. Additional mice (3 or 4 per group) were also vaccinated intramuscularly at day 0 and day 12 with: (i) a 10-μg dose of the first circular RNA formulated with lipid nanoparticles; (ii) a 10-μg dose of the second circular RNA formulated with lipid nanoparticles; or (iii) PBS.

Blood collection was as described in Experiment 1. The presence of RBD-specific IgG by ELISA was determined as described in Experiment 1.

Individual serum samples were assayed for the presence of HA-specific IgG by ELISA. ELISA plates were coated overnight at 4° C. with HA recombinant protein (Sino Biological, 11085-V08B; 100 ng) and plates were processed as described in Experiment 1. The optical density of each serum sample was divided by that of the background (plates coated with HA, incubated only with secondary antibody). The fold over background of each sample was plotted.

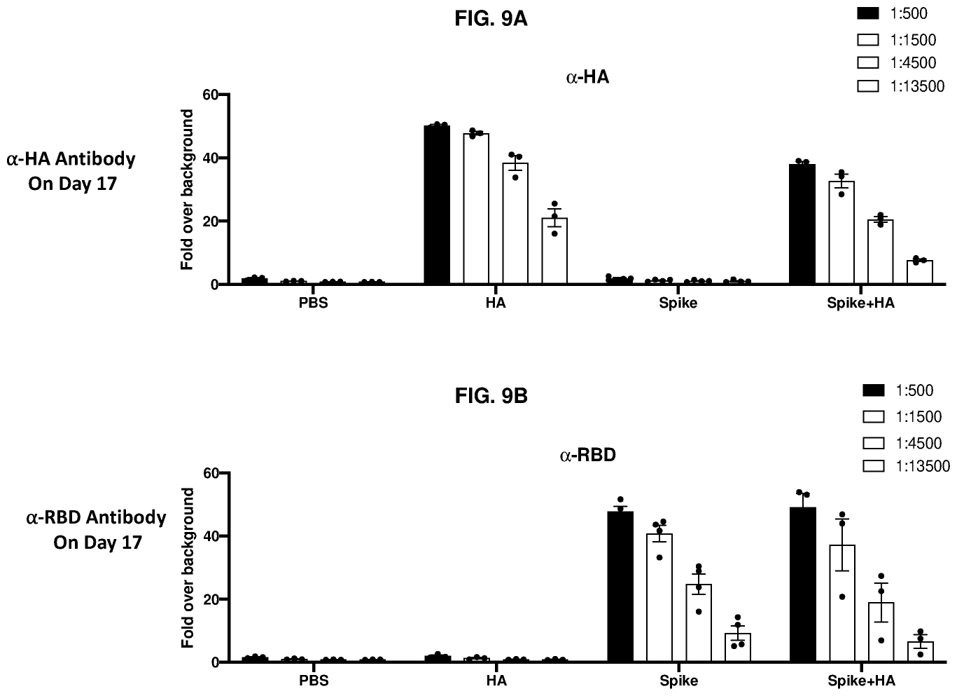

The results showed that anti-RBD and anti-HA antibodies were obtained at 17 days post prime (i.e., 17 days after injection with the first circular RNA preparation (FIGS. 9A and 9B).

The results also showed that circular RNA preparations comprising two circular RNAs encoding different immunogens induce an immunogen-specific immune response in mice.

### Experiment 3

The immunogenicity of a circular RNA preparation comprising (a) a circular RNA encoding a SARS-CoV-2 Spike immunogen and (b) a circular RNA encoding an Influenza hemagglutinin (HA) immunogen, formulated in lipid nanoparticles, was evaluated in a mouse model. Production of antibodies to the SARS-CoV-2 Spike and Influenza HA immunogens were also evaluated in the mouse model.

A first circular RNA encoding a SARS-CoV-2 Spike immunogen (Nucleic acid SEQ ID NO: 54; Amino acid SEQ ID NO: 53) was designed and produced and purified by the methods described herein. A second circular RNA was designed to include an IRES followed by an ORF encoding hemagglutinin (HA) from Influenza A H1N1, A/California/07/2009 (Nucleic acid SEQ ID NO: 60; Amino acid SEQ ID NO: 59) and produced and purified by the methods described herein. The first circular RNA and the second circular RNA were mixed together to obtain a mixture. This mixture was then formulated with lipid nanoparticles to obtain a first circular RNA preparation. The first circular RNA and the second circular RNA were also separately formulated with lipid nanoparticles, and then mixed together to obtain a second circular RNA preparation.

Three mice were vaccinated intramuscularly with the first circular RNA preparation at day 0 (for a total dose of 10 μg Spike+10 μg HA) and with the second circular RNA preparation (for a total dose of 10 μg Spike+10 μg HA) at day 12. Additional mice (3 or 4 per group) were also vaccinated intramuscularly at day 0 and day 12 with: (i) a 10-μg dose of the first circular RNA formulated with lipid nanoparticles; (ii) a 10-μg dose of the second circular RNA formulated with lipid nanoparticles; or (iii) PBS.

Blood collection was as described in Experiment 1 Serum was separated from whole blood by centrifugation at 1200 g for 30 minutes at 40C. Individual serum samples were assayed for the presence of RBD (i.e., RBD of Spike)-specific IgG by ELISA as described in Experiment 1.

Individual serum samples were assayed for the presence of HA-specific IgG by ELISA. ELISA plates were coated overnight at 4° C. with HA recombinant protein (Sino Biological, 11085-V08B; 100 ng) and plates were processed as described in Experiment 1. The optical density of each serum sample was divided by that of the background (plates coated with HA, incubated only with secondary antibody). The fold over background of each sample was plotted.

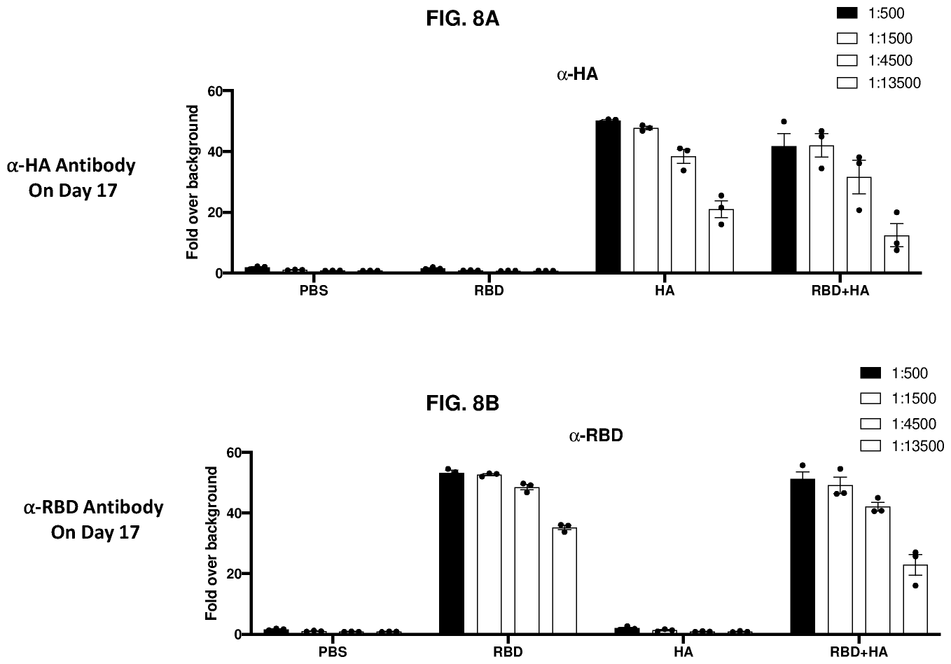

The results showed that anti-RBD antibodies and anti-HA antibodies were obtained at 17 days post prime (i.e., 17 days after injection with the first circular RNA preparation (FIGS. 8A and 8B).

The results also showed that circular RNA preparations comprising two circular RNAs encoding different immunogens induced immunogen-specific immune responses in mice.

### Example 10: Immunogenicity of a Circular RNA Comprising Multiple Immunogens in a Mouse Model

This Example describes the immunogenicity of a circular RNA comprising multiples immunogens.

This example also describes production of antibodies in a mouse model to multiple immunogens encoded by a single circular RNA.

### Experiment 1

In this Example, a circular RNA is designed to include an IRES followed by an ORF encoding GLuc, a stop codon, a spacer, an IRES, an ORF encoding SARS-CoV-2 RBD immunogen, and a stop codon, produced and purified as described in Example 8. As controls, the following circular RNAs are produced as described above: (i) a circular RNA with an IRES and ORF encoding a SARS-CoV-2 RBD immunogen; (ii) a circular RNA with an IRES and ORF encoding GLuc.

The circular RNAs are formulated with lipid nanoparticles to obtain a circular RNA preparation.

Three mice per group are vaccinated intramuscularly with a 10 μg or 20 μg total dose of circular RNA preparation at day 0 and at day 12.

Blood collection is as described in Example 9. The presence of RBD-specific IgG by ELISA is determined as described in Example 9. GLuc activity is measured as described in Example 9.

### Experiment 2

The immunogenicity of a circular RNA preparation comprising a circular RNA designed to include an IRES followed by an ORF encoding a SARS-CoV-2 RBD immunogen, a stop codon, a spacer, an IRES, an ORF encoding a MERS RBD immunogen, and a stop codon, formulated in lipid nanoparticles, is evaluated in a mouse model. Production of antibodies to the SARS-CoV-2 RBD and MERS RBD immunogens are also evaluated in the mouse model.

This circular RNA is then formulated with lipid nanoparticles as described in Example 7 to obtain a circular RNA preparation.

Mice are vaccinated intramuscularly or intradermally with the circular RNA preparation with amounts of 5 μg, 10 μg, 20 μg, or 50 μg at day 0 and again at least one day after the initial administration.

Blood collection is as described in Experiment 1. The presence of SARS-CoV-2 RBD-specific IgG by ELISA is determined as described in Experiment 1. The presence of MERS RBD-specific IgG is also determined by ELISA.

Individual serum samples are assayed for the presence of anti-SARS-CoV-2 RBD binding antibodies, anti-MERS RBD binding antibodies, neutralizing antibodies against the SARS-CoV-2 RBD immunogen, neutralizing antibodies against the MERS RBD immunogen, a cellular response to the SARS-CoV-2 immunogen, and a cellular response to the MERS RBD immunogen.

### Example 11: Evaluation of T Cell Responses

An ELISpot assay is used to detect the presence of SARS-CoV-2 Spike or RBD-specific T cells or Influenza HA-specific T cells. This assay is performed on the following groups of mice from Example 9:
1. RBD
2. GLuc
3. HA
4. Spike
5. RBD+HA
6. Spike+HA
7. PBS

Mice spleens are harvested on day 30 post boost (i.e., 30 days after injection with the first circular RNA preparation) and processed into a single cell suspension. Splenocytes are plated at 0.5M cells per well on IFN-g or IL-4 ELISpot plates (ImmunoSpot). Splenocytes are either left unstimulated or stimulated with SARS CoV-2 and HA peptide pools (JPT, PM-WCPV-SRB and PM-IFNA_HACal). ELISPOT plates are processed according to manufacturer's protocol.

### Example 12: Evaluation of Antibody Response in Mice Administered Circular RNA Encoding Multiple Immunogens

This example demonstrates an antibody response resulting from administration of a circular RNA encoding the expression of the multiple immunogens.

A hemagglutination inhibition assay (HAI) was used to measure anti-Influenza HA antibodies that prevent hemagglutination in serum from mice. Mice were administered a preparation of circular RNA each of which was designed and produced the methods described herein, and which encode for the expression of: a SARS-CoV-2 RBD immunogen, a SARS-CoV-2 Spike immunogen, an Influenza HA immunogen, a SARS-CoV-2 RBD immunogen and an Influenza HA immunogen, a SARS-CoV-2 RBD immunogen and a GLuc protein, or a SARS-CoV-2 RBD immunogen and a SARS-CoV-2 Spike immunogen. Blood collection was as described in Example 9, Experiment 1 and was performed on day 2 and day 17 after injection.

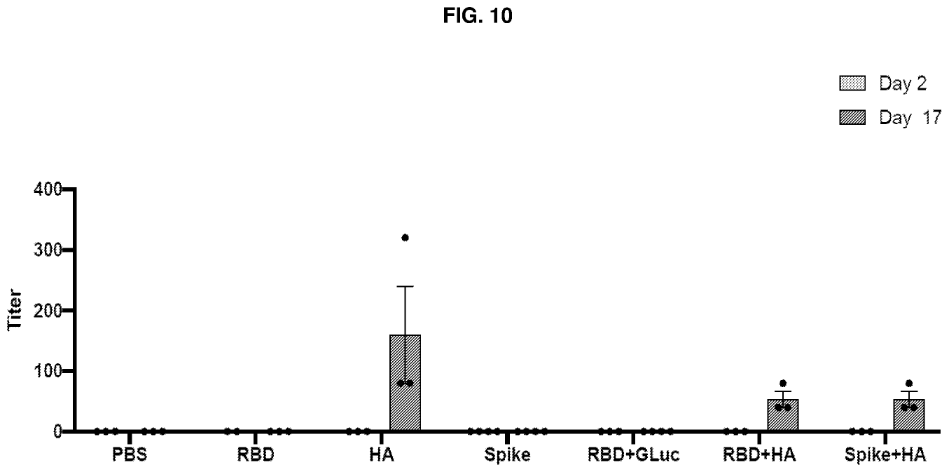

Two-fold serial dilutions of the collected sample from mice on day 2 and day 17 were prepared. A fixed amount of influenza virus with known hemagglutinin (HA) titer was added to every well of a 96-well plate, to a concentration equivalent to 4 hemagglutinin units, with the exception of the serum control wells, where no virus was added. The plate was allowed to stand at room temperature for 60 minutes, after which the red blood cell samples were added and allowed to incubate at 4° C. for 30 minutes. The highest serum dilution that prevented hemagglutination was determined to be the HAI titer of the serum. The sample collected on day 17 showed HAI titer in samples that were administered circular RNA preparations encoding the Influenza HA immunogen when it was administered alone or when administered in combination with SARS-CoV-2 immunogens e.g., RBD or Spike (FIG. 10). HAI titers on day 17 were not seen from samples where HA immunogen had not been administered e.g., the SARS-CoV-2 RBD immunogen alone or SARS-CoV-2 Spike immunogen alone.

### Example 13: Expression of an Adjuvant from Circular RNA in Mammalian Cells

This example demonstrates expression of a polypeptide adjuvant from circular RNA in mammalian cells.

In this example, a circular RNA was designed to include an IRES, an ORF encoding the adjuvant IL-12 (Nucleic acid SEQ ID NO: 217; Amino acid SEQ ID NO: 218), and two spacer elements flanking the IRES-ORF. As a control, a circular RNA including an IRES, an ORF encoding a SARS-CoV-2 RBD immunogen, and two spacer elements flanking the IRES-ORF was used. The circular RNAs were produced and purified according to the methods described herein.

Purified circular RNAs (0.1 and 1 picomoles) were transfected into HeLa cells (10,000 cells per well) using Lipofectamine MessengerMax (Invitrogen LMRNA001) according to the manufacturer's instructions.

IL-12 expression was measured using an IL-12 specific ELISA (ThermoFisher, BMS6004) in cell culture supernatant. Data are shown as Mean and SEM values of two replicates.

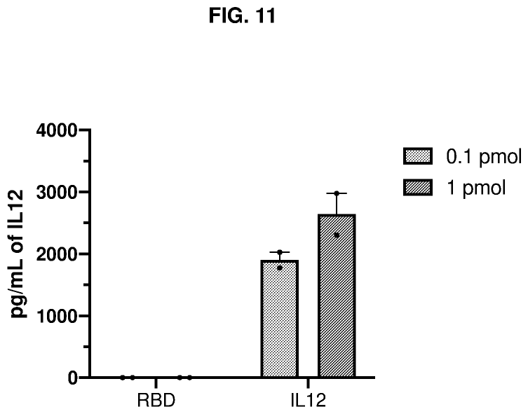

The results showed that IL-12 encoded by circular RNA was expressed by HeLa cells and not in the control (FIG. 11). This example shows that the IL-12 adjuvant was expressed from circular RNA in mammalian cells.

### Example 14: In Vivo Expression of an Adjuvant from Circular RNA in Mouse Model

This example demonstrates in vivo expression of a polypeptide adjuvant from a circular RNA.

In this example, the following circular RNAs were produced and purified according to the methods described herein: (i) a first circular RNA with an IRES and an ORF encoding an IL-12 adjuvant (Nucleic acid SEQ ID NO: 217 Amino acid SEQ ID NO: 218); and (ii) a second circular RNA with an IRES and ORF encoding a SARS-CoV-2 RBD immunogen with an N-terminal Gluc signal sequence (Nucleic acid SEQ ID NO: 56; Amino acid SEQ ID NO: 55). The first circular RNA and the second circular RNA were each separately formulated with lipid nanoparticles and then mixed together to obtain a first circular RNA preparation.

To formulate the circular RNA or mRNA with a lipid nanoparticle, circular RNA or mRNA was diluted in 25 mM acetate buffer pH=4 (filtered through 0.2 μm filter) to a concentration of 0.2 μg/μL. Lipid nanoparticles (LNPs) were formulated by first dissolving the ionizable lipid (e.g., ALC0315), cholesterol, DSPC, and DMG-PEG2000 in ethanol (filtered through 0.2 μm sterile filter) in a molar ratio of 50/38.5/10/1.5 mol %. The final ionizable lipid/RNA weight ratio was 8/1 w/w. The lipid and RNA solutions were mixed in a micromixer chip using microfluidics system with a flow rate ratio of 3/1 buffer/ethanol and a total flow rate of 1 ml/min. The LNPs were then dialyzed in PBS pH=7.4 for 3 h to remove ethanol. The RNA concentration inside the LNPs and the encapsulation efficiency were measured using Ribogreen® assay. If necessary, the LNPs were concentrated down to the desired RNA concentration using Amicon centrifugation filters, 100 kDa cut off. The size, concentration, and charge of the particles were measured using Zetasizer Ultra (Malvern Pananaytical). The RNA concentration was adjusted with PBS to a final concentration of 0.1 or 0.2 μg/μl. For formulations containing two RNA sequences the RNAs were either mixed before formulating in LNPs or after each RNA was formulated separately. For in vivo experiments, the final RNA formulated in LNPs were filtered through sterile 0.2 μm regenerated cellulose filters.

Three mice were vaccinated intramuscularly at day 0 with the first circular RNA preparation (10 μg dose). As controls, three mice per group were vaccinated intramuscularly with: (i) the second circular RNA formulated with lipid nanoparticles (10 μg dose) (i.e., the second circular RNA preparation); or (ii) PBS. Blood collection from each mouse was by submandibular drawing. Blood was collected from each mouse into dry-anticoagulant free-tubes at 2 days after administration of circular RNA or PBS. Serum was separated from whole blood by centrifugation at 1200 g for 30 minutes at 4° C. Individual serum samples were assayed for the presence of IL12 using a cytokine bead array (Biolegend, 749622). Data are shown as Mean and SEM values of three replicates.

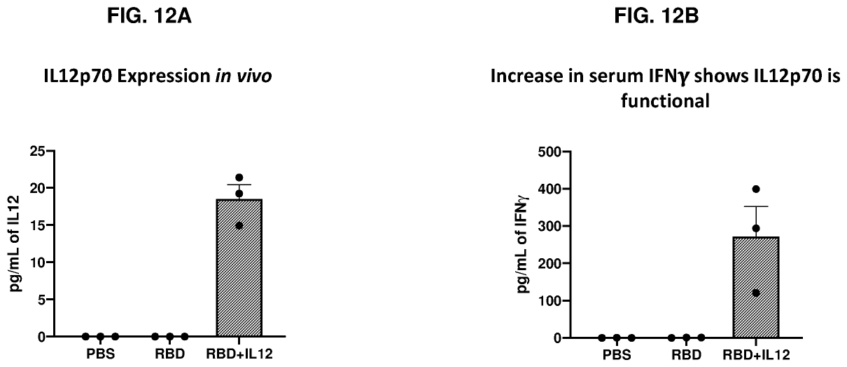

The results showed that IL-12 expression was detected in serum at 2 days after injection with the first circular RNA preparation but was not detected after injection with either of the controls (FIG. 12A).

To determine if the expressed IL12 is functional, IFN-γ (directly downstream of IL12 signaling) production in the serum was measured 2 days after injection with the circular RNA preparations. IFN-γ production was detected in serum in the same cytokine bead array assay described herein (Biolegend, 749622). Data are shown as Mean and SEM values of 3 replicates.

The results showed an increase in serum IFN-γ, indicating that circular RNA expressed IL12 is functional (FIG. 12B).

### Example 15: Induction of Immunogenicity in a Mouse Model by Co-Administration of an Immunogen and an Adjuvant Encoded by a Plurality of Circular RNAs

This example demonstrates the immunogenicity induced by administration of a plurality of circular RNAs to a subject. One circular RNA administered encodes an immunogen. Another circular RNA administered encodes a polypeptide adjuvant.

In this example, the following circular RNAs were produced and purified according to the methods described herein: (i) a first circular RNA with an IRES and an ORF encoding an IL-12 adjuvant (Nucleic acid SEQ ID NO: 217; Amino acid SEQ ID NO: 218); and (ii) a second circular RNA with an IRES and ORF encoding a SARS-CoV-2 RBD immunogen with an N-terminal Gluc signal sequence (Nucleic acid SEQ ID NO: 56; Amino acid SEQ ID NO: 55). The first circular RNA and the second circular RNA were each separately formulated with lipid nanoparticles and then mixed together to obtain a first circular RNA preparation.

To formulate the circular RNA or mRNA with a lipid nanoparticle, circular RNA or mRNA was diluted in 25 mM acetate buffer pH=4 (filtered through 0.2 μm filter) to a concentration of 0.2 μg/μL. Lipid nanoparticles (LNPs) were formulated by first dissolving the ionizable lipid (e.g., ALC0315), cholesterol, DSPC, and DMG-PEG2000 in ethanol (filtered through 0.2 μm sterile filter) in a molar ratio of 50/38.5/10/1.5 mol %. The final ionizable lipid/RNA weight ratio was 8/1 w/w. The lipid and RNA solutions were mixed in a micromixer chip using microfluidics system with a flow rate ratio of 3/1 buffer/ethanol and a total flow rate of 1 ml/min. The LNPs were then dialyzed in PBS pH=7.4 for 3 h to remove ethanol. The RNA concentration inside the LNPs and the encapsulation efficiency were measured using Ribogreen® assay. If necessary, the LNPs were concentrated down to the desired RNA concentration using Amicon centrifugation filters, 100 kDa cut off. The size, concentration, and charge of the particles were measured using Zetasizer Ultra (Malvern Pananaytical). The RNA concentration was adjusted with PBS to a final concentration of 0.1 or 0.2 μg/μL. For formulations containing two RNA sequences the RNAs were either mixed before formulating in LNPs or after each RNA was formulated separately. For in vivo experiments, the final RNA formulated in LNPs were filtered through sterile 0.2 μm regenerated cellulose filters.

Three mice were vaccinated intramuscularly at day 0 and day 14 with the first circular RNA preparation (2.5 μg dose per circular RNA per injection). As controls, three mice per group were vaccinated intramuscularly with: (i) the second circular RNA formulated with lipid nanoparticles (2.5 μg dose per injection) (i.e., the second circular RNA preparation); or (ii) PBS. Mice were euthanized 22 days post the first dose and splenocytes processed into a single cell suspension. Splenocytes were either left unstimulated or stimulated with RBD peptide pool (JPT, PM-WCPV-S-RBD-2) for 1 hour. Protein transport inhibitors (Monensin, BD 554724) and Brefeldin A, BD 555029)) were then added to the media followed by 5 more hours of culture. The cells were stained using the BD fixation and permeabilization kit (BD, 554714) according to the manufacturer's protocol. Antibodies used: viability dye (ThermoFisher, L₁₀₁₁), CD8 (ThermoFisher, MA5-16759) and CD44 (Biolegend, 103026). Stained cells were analyzed by flow cytometry.

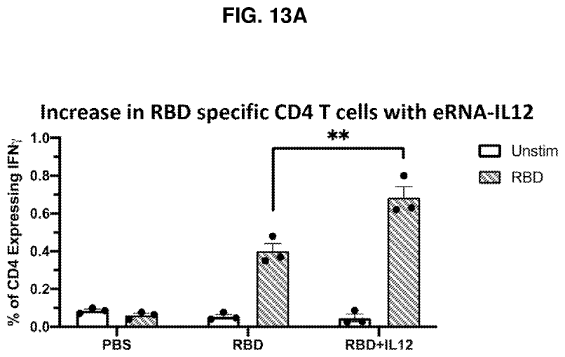

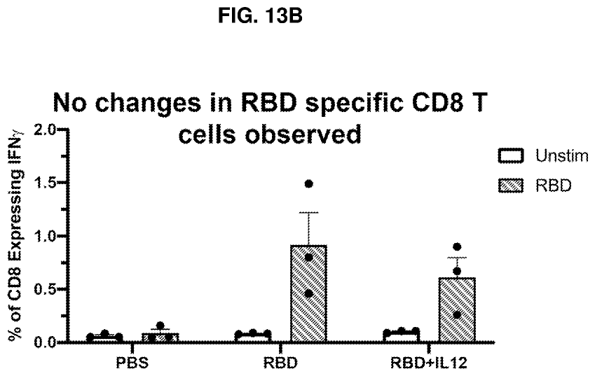

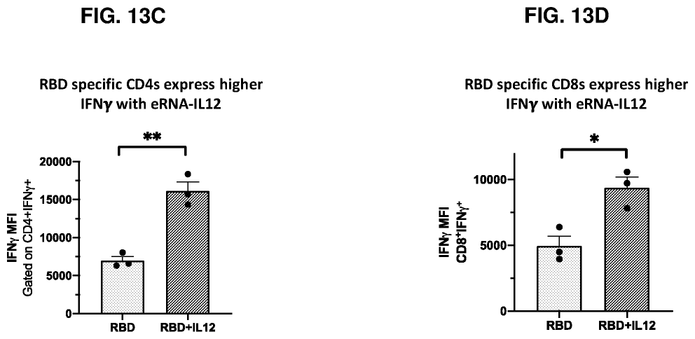

The data shows that the first circular RNA preparation increased the number of RBD specific CD4 T cells relative to the control, i.e., the second circular RNA preparation (FIG. 13A) and that no changes in RBD specific CD8 T cells were observed (FIG. 13B). In addition, the first circular RNA preparation increased the amount of IFN-γ production by both CD4 and CD8 T cells (FIGS. 13C, 13D). This shows that circular RNA expressing IL12 is acting as an adjuvant boosting cellular immune response elicited by circular RNA expressing an immunogen. Data shown as mean and SEM of 3 replicates. FIG. 13A: Asterisks denotes statistical significance as determined by a two-way RM ANOVA protected Tukey's post hoc test. FIGS. 13C, 13D: Asterisks denotes statistical significance as determined by unpaired t-test.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 16: In Vivo Expression of Non-Secreted SARS-CoV-2 Immunogen from RNA in Non-Human Primate Model

This example demonstrates in vivo expression a non-secreted of SAR-CoV-2 immunogen from circular RNA in a non-human primate (NHP).

Circular RNA was designed to include an internal ribosome entry site (IRES) and a nucleotide sequence encoding a SAR-CoV-2 spike immunogen. The DNA construct was designed to include an IRES, a polynucleotide cargo, and a spacer element. In this example, the construct was designed to include a CVB3 IRES (SEQ ID NO: 45) and a nucleotide sequence encoding a SARS-CoV-2 spike ORF (SEQ ID NO: 237) as the polynucleotide cargo.

In this example, the circular RNA was generated by self-splicing using a method described herein. Unmodified linear RNA was synthesized by in vitro transcription using T7 RNA polymerase from a DNA template including the motifs listed above in the presence of 7.5 mM of NTP. Synthesized linear RNA was purified with an RNA clean up kit (New England Biolabs, T2050). Self-splicing occurred during transcription; no additional reaction was required. The circular RNA was purified by urea polyacrylamide gel electrophoresis (Urea-PAGE) or by reversed phase column chromatography.

Purified circular RNA was formulated into a lipid nanoparticle (LNP) to obtain a circular RNA preparation. Briefly, circular RNA was diluted in 25 mM acetate buffer pH=4 (filtered through 0.2 um filter) to a concentration of 0.2 μg/μL. LNPs were formulated by first dissolving the ionizable lipid (e.g., ALC0315), cholesterol, DSPC, and DMG-PEG2000 in ethanol (filtered through 0.2 um sterile filter) in a molar ratio of 50/38.5/10/1.5 mol %. The final ionizable lipid/RNA weight ratio was 6/1 w/w. The lipid and RNA solutions were mixed in a micromixer chip using microfluidics system with a flow rate ratio of 3/1 buffer/ethanol and a total flow rate of 1 ml/min. The LNPs were then dialyzed in PBS pH=7.4 for 3 hours to remove ethanol. The LNPs were concentrated to the desired RNA concentration using Amicon centrifugation filters, 100 kDa cut off, as necessary.

Three cynomolgus monkeys (n=3) per group were administered either a 30 μg or 100 μg dose of LNP-formulated circular RNA via intramuscular injection at day 0 (prime) and day 28 (boost). At 6 hours post-prime, serum samples were collected from each monkey. Spike levels were measured using a SARS-CoV-2 Spike immunoassay according to manufacturer's protocol (MDS, S-PLEX SARS-CoV-2 Spike Kit, K150ADJS-2).

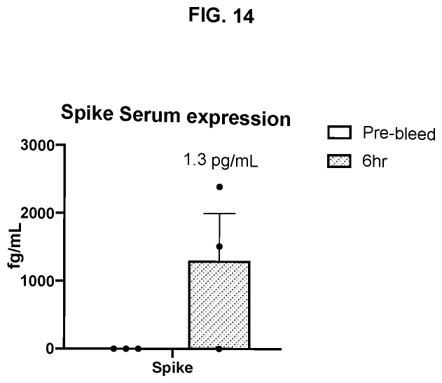

Spike immunogen was detected in serum of monkeys that received 100 μg of LNP-formulated circular RNA at 6 hours post prime (FIG. 14, data shown as the mean of three animals per group).

### Example 17: In Vivo Expression of Secreted Immunogen from Circular RNA in Non-Human Primate Model

This example demonstrates in vivo expression of a secreted SAR-CoV-2 immunogen from circular RNA in a non-human primate (NHP).

Circular RNA was designed to include an IRES and a nucleotide sequence encoding a SARS-CoV-2 RBD immunogen. The DNA construct was designed to include an IRES, a polynucleotide cargo, and a spacer element. In this example, the construct was designed to include an EMCV IRES (SEQ ID NO: 31) and a nucleotide sequence encoding a Gaussia luciferase (Glue) secretion signal sequence and a SARS-CoV-2 RBD immunogen fused to a T4 Foldon domain (SEQ ID NO: 303) as the polynucleotide payload. The circular RNA was produced as described in Example 16.

Circular RNA was formulated in LNP as described in Example 16 (LNP-formulated circular RNA). Circular RNA was also formulated by admixing with an equal volume of AddaSO3™ adjuvant solution (adjuvanted circular RNA).

Three cynomolgus monkeys (n=3) per group were administered either a 30 μg or 100 μg dose of LNP-formulated circular RNA, or a 1000 μg dose of adjuvanted circular RNA via intramuscular injection at day 0 (prime) and day 28 (boost). At 6 hours, Day 1, Day 4, Day 6 post-prime, serum samples were collected from each monkey. SARS-CoV-2 RBD immunogen fused to a T4 foldon multimerization domain levels were measured using a SARS-CoV-2 Spike immunoassay according to manufacturer's protocol (MDS, S-PLEX SARS-CoV-2 Spike Kit, K150ADJS-2).

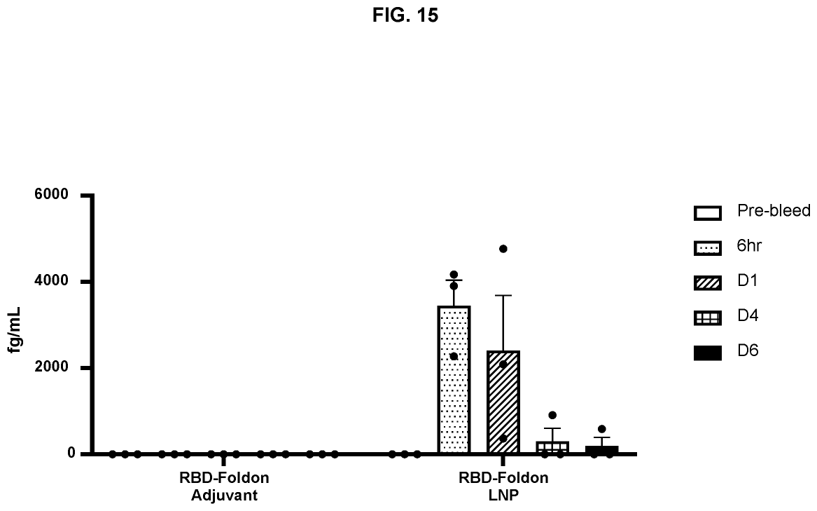

SARS-CoV-2 RBD immunogen fused to a T4 foldon multimerization domain expression was not detected in serum of monkeys that were administered adjuvanted circular RNAs. SARS-CoV-2 RBD immunogen fused to a T4 foldon multimerization domain was detected in serum of monkeys that received 100 μg of LNP-formulated circular RNA (FIG. 15, data shown as the mean of three animals per group). SARS-CoV-2 RBD immunogen fused to a T4 foldon multimerization domain levels of ˜3500 fg/mL were detected at 6 hours post-prime, with a SARS-CoV-2 RBD immunogen fused to a T4 foldon multimerization domain concentrations decreasing over the course of the 6 days during which samples were collected.

### Example 18: Immunogenicity of Immunogens from Circular RNA in Non-Human Primate Model

This example demonstrates circular RNA encoding a SARS-CoV-2 immunogen induces an immunogen-specific response in a non-human primate (NHP).

Serum samples were isolated from monkeys administered 30 μg or 100 μg dose of LNP-formulated circular RNA, or 1000 μg dose of adjuvanted circular RNA as described in Examples 16 and 17, at Days 14 and 42 post-prime.

Binding antibody was detected using a SARS-CoV-2 Spike immunoassay according to manufacturer's protocol MDS. S-PLEX SARS-CoV-2 Spike Kit, K150ADJS-2). NHP serum was diluted at 1:1000 or 1:5000 or 1:50 000. Binding antibody concentration was interpolated using the pooled serum standard and results were reported as Geometric Mean international Units per mL.

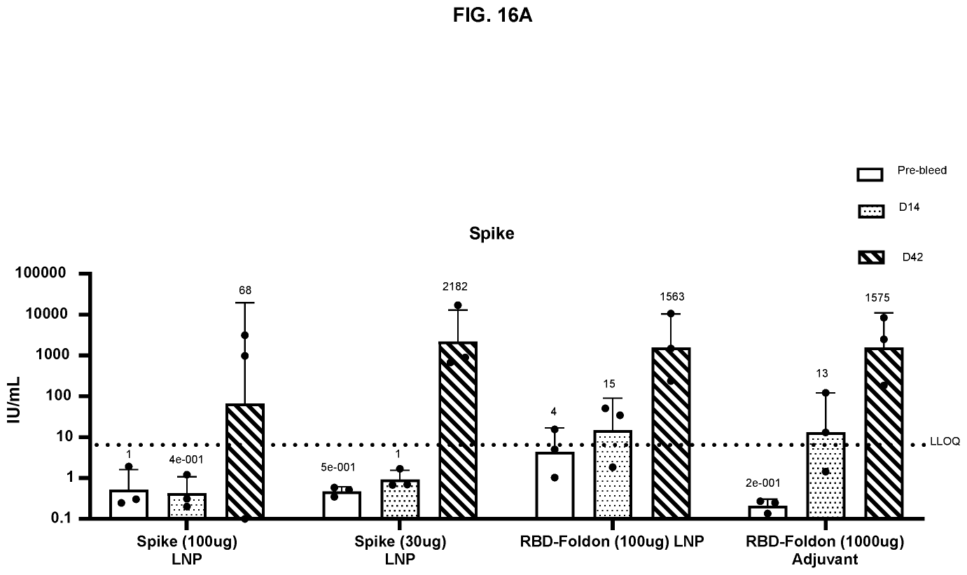

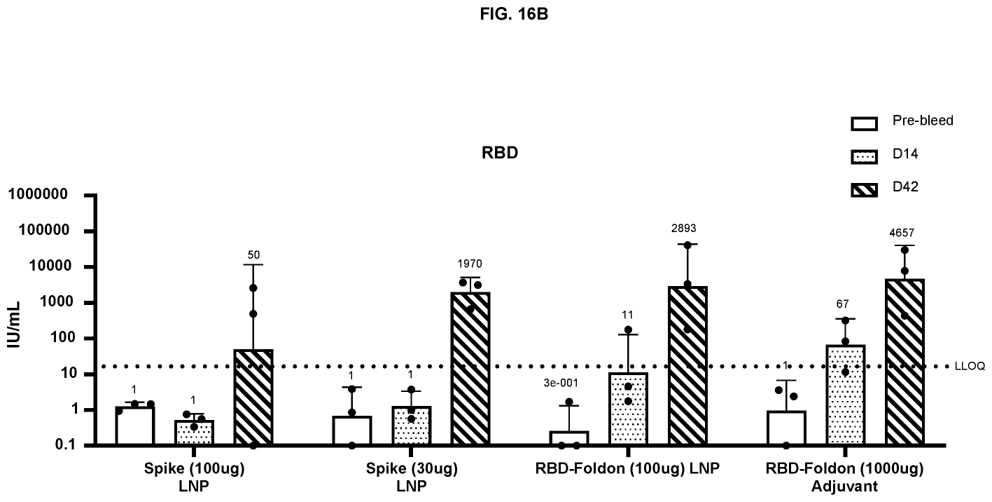

FIG. 16A shows the geometric mean IU/mL of Spike specific antibody at pre-bleed, Day 14 and Day 42 post-immunization with LNP-formulated circular RNAs (Spike (30 μg and 100 μg), SARS-CoV-2 RBD immunogen fused to a T4 foldon multimerization domain (100 μg)), and adjuvanted circular RNA (SARS-CoV-2 RBD immunogen fused to a T4 foldon multimerization domain (1000 μg)). The results show that LNP-formulated circular RNA encoding a SARS-CoV-2 Spike immunogen primed Spike-specific binding antibodies at Day 42 post-prime at 100 μg and 30 μg dose levels. The results also show that LNP-formulated circular RNA encoding a SARS-CoV-SARS-CoV-2 RBD immunogen fused to a T4 foldon multimerization domain primed Spike-specific binding antibodies at Day 42 post-prime, and that adjuvanted circular RNA encoding a SARS-CoV-2 RBD immunogen fused to a T4 foldon multimerization domain primed similar levels of Spike-specific binding antibodies.

FIG. 16B shows the geometric mean IU/mL of RBD specific antibody at pre-bleed, Day 14 and Day 42 post-immunization with LNP-formulated circular RNAs (Spike (30 μg and 100 μg), SARS-CoV-2 RBD immunogen fused to a T4 foldon multimerization domain (100 μg)), and adjuvanted circular RNA (SARS-CoV-2 RBD immunogen fused to a T4 foldon multimerization domain (1000 μg)). The results show that LNP-formulated circular RNA encoding a SARS-CoV-2 Spike immunogen primed RBD-specific binding antibodies at Day 42 post-prime at 100 μg and 30 μg dose levels. The results also show that LNP-formulated circular RNA primed RBD-specific binding antibodies at Day 42 post-prime, and that adjuvanted circular RNA encoding a SARS-CoV-2 RBD immunogen fused to a T4 foldon multimerization domain primed similar levels of RBD-specific binding antibodies.

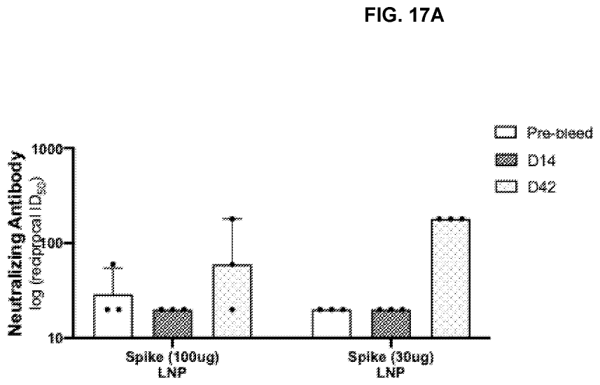

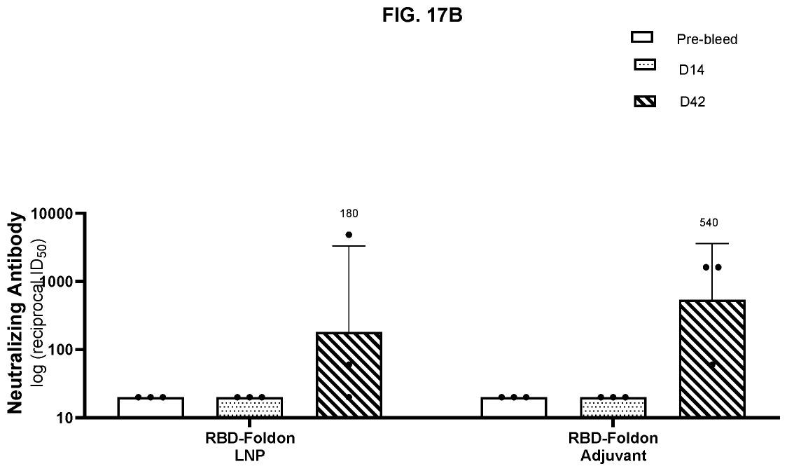

The neutralizing antibody titer from serum collected on pre-bleed, Day 14 and Day 42 post-prime was tested in a Plaque Reduction Neutralization Test (PRNT). Briefly serum was serially diluted, mixed with SARS-CoV-2 viral stock and placed on Vero E6 cells. Plates were overlayed with low-melting point agarose and incubated for 3 days, followed by fixation and staining with crystal violet. The neutralization titer was reported as D50: the dilution at which the serum reduces the number of plaques by fifty percent (50%). Data are shown in FIGS. 17A and 17B as geometric mean neutralizing titer at pre-bleed, and Day 14 and Day 42 post-boost.

FIG. 17A shows that DLNP-formulated circular RNA encoding Spike (30 μg and 100 μg) primed SARS-CoV-2 neutralizing antibodies at Day 42.

FIG. 17B shows that both LNP-formulated circular RNA encoding a SARS-CoV-2 RBD immunogen fused to a T4 foldon multimerization domain and adjuvanted circular RNA encoding a SARS-CoV-2 RBD immunogen fused to a T4 foldon multimerization domain primed SARS-CoV-2 neutralizing antibody.

### Example 19: T Cell Responses of Immunogens from Circular RNA in Non-Human Primate Model

Peripheral blood mononuclear cells (PBMCs) are harvested and frozen pre-immunization and at D42 post-immunization. PBMCs are thawed and an ELISpot assay is used to detect the presence of SARS-CoV-2 RBD-specific T cells. 0.2 M cells are plated per well on IFN-γ or IL-4 ELISpot plates (ImmunoSpot) and are either left unstimulated or stimulated with SARS-CoV-2 peptide pools (JPT, PM-WCPVS-2). ELISpot plates are processed according to manufacturer's protocol.

### Numbered Embodiments

[1] A circular polyribonucleotide comprising an open reading frame encoding a coronavirus immunogen, wherein the coronavirus immunogen comprises an amino acid sequence having at least 95% sequence identity with the amino acid sequence of any one of SEQ ID NOs: 63-111 and 283-291.
[2] The circular polyribonucleotide of embodiment [1], wherein the coronavirus immunogen is a RBD immunogen having at least 95% identity with the amino acid sequence of any one of SEQ ID NOs: 63-68, 74, 79, 81-86, and 98-111.
[3] The circular polyribonucleotide of [1], wherein the coronavirus immunogen is a Spike immunogen having at least 95% identity with the amino acid sequence of any one of SEQ ID NOs: 69-73, 75-78, 80, 87-97 and 283-286.
[4] The circular polyribonucleotide of embodiment [1 [. wherein the coronavirus immunogen is a nonstructural protein (nsp) immunogen having at least 95% identity with the amino acid sequence of any one of SEQ ID NOs: 291-295.
[5] The circular polyribonucleotide of embodiment [1], wherein the coronavirus immunogen comprises an amino acid sequence of any one of SEQ ID NOs: 63-111 and 283-291.
[6] The circular polyribonucleotide of any one of embodiments [1]-[5], wherein the open reading frame comprises a nucleic acid sequence having at least 95% sequence identity with the nucleic acid sequence of any one of SEQ ID NOs: 112-174 and 292-300.
[7] The circular polyribonucleotide of embodiment [6], wherein the coronavirus immunogen is a RBD immunogen having at least 95% identity with the nucleic acid sequence of any one of SEQ ID NOs: 112-117, 123, 128, 133-138, and 163-174.
[8] The circular polyribonucleotide of embodiment [6], wherein the coronavirus immunogen is a Spike immunogen having at least 95% identity with the nucleic sequence of any one of SEQ ID NOs: 118-122, 124-127, 129-132, 139-162, and 287-291.
[9] The circular polyribonucleotide of embodiment [6], wherein the coronavirus immunogen is a nsp having at least 95% identity with the nucleic sequence of any one of SEQ ID NOs: 296-300.
[10] The circular polyribonucleotide of embodiment [6], wherein the open reading frame comprises a nucleic acid sequence of any one of SEQ ID NOs: 112-174 and 292-300.
[11] The circular polyribonucleotide of any one of embodiment [1]-[10], wherein the open reading frame encoding the coronavirus immunogen is operably linked to an IRES.
[12] The circular polyribonucleotide of any one of embodiment [1]-[11], wherein the open reading frame encoding the coronavirus immunogen encodes a second polypeptide.
[13] The circular polyribonucleotide of embodiment [12], wherein the coronavirus immunogen and the second polypeptide are separated by a polypeptide linker, a 2A self-cleaving peptide, a protease cleavage site, or 2A self-cleaving peptide in tandem with a protease cleavage site.
[14] The circular polyribonucleotide of embodiment [13], wherein the protease cleavage site is a furin cleavage site.
[15] The circular polyribonucleotide of any one of embodiments [1]-[11], wherein the circular polyribonucleotide further comprises a second open reading frame encoding a second polypeptide operably linked to a second IRES.
[16] The circular polyribonucleotide of any one of embodiments [12]-[15], wherein the second polypeptide is a polypeptide immunogen.
[17] The circular polyribonucleotide of embodiment [16], wherein the second polypeptide is a viral immunogen.
[18] The circular polyribonucleotide of embodiment [17], wherein the second polypeptide is a coronavirus immunogen.
[19] The circular polyribonucleotide of embodiment [18], wherein the second coronavirus immunogen comprises an amino acid sequence of any one of SEQ ID NOs: 1-10, 53, 55, 57, 63-111, and 283-291.
[20] The circular polyribonucleotide of embodiment [17], wherein the second polypeptide is an influenza immunogen.
[21] The circular polyribonucleotide of any one of embodiments [12]-[15], wherein the second polypeptide is a polypeptide adjuvant.
[22] The circular polyribonucleotide of embodiment [18], wherein the adjuvant is a cytokine, a chemokine, a costimulatory molecule, an innate immune stimulator, a signaling molecule, a transcriptional activator, a cytokine receptor, a bacterial component, or a component of the innate immune system.
[23] The circular polyribonucleotide of any one of embodiments [1]-[22], wherein the circular polyribonucleotide further comprises a non-coding ribonucleic acid sequence that is an innate immune system stimulator.
[24] The circular polyribonucleotide of embodiment [23], wherein the innate immune system stimulator is selected from a GU-rich motif, an AU-rich motif, a structured region comprising dsRNA, or an aptamer.
[25] A circular polyribonucleotide comprising a first sequence encoding a coronavirus immunogen and a second sequence encoding a polypeptide adjuvant.
[26] The circular polyribonucleotide of embodiment [25], wherein the sequence encoding the coronavirus immunogen is operably linked to a first IRES and the sequence encoding the polypeptide adjuvant is operably linked to a second IRES.
[27] The circular polyribonucleotide of embodiment [25], wherein the coronavirus immunogen and the polypeptide adjuvant are encoded by a single open-reading frame operably linked to an IRES.
[28] The circular polyribonucleotide of embodiment [27], wherein coronavirus immunogen and the polypeptide adjuvant are separated by a polypeptide linker, a 2A self-cleaving peptide, a protease cleavage site, or 2A self-cleaving peptide in tandem with a protease cleavage site.
[29] The circular polyribonucleotide of any one of embodiments [25]-[28], wherein polypeptide adjuvant is a cytokine, a chemokine, a costimulatory molecule, an innate immune stimulator, a signaling molecule, a transcriptional activator, a cytokine receptor, a bacterial component, or a component of the innate immune system.
[30] The circular polyribonucleotide of any one of embodiments [25]-[29], wherein the second coronavirus immunogen comprises an amino acid sequence of any one of SEQ ID NOs: 1-10, 53, 55, 57, 63-111, and 283-291.
[31] A circular polyribonucleotide comprising an open reading frame encoding a coronavirus immunogen and a non-coding ribonucleic acid sequence that is an innate immune system stimulator.
[32] The circular polyribonucleotide of embodiment [31], wherein the innate immune system stimulator is selected from a GU-rich motif, an AU-rich motif, a structured region comprising dsRNA, or an aptamer.
[33] The circular polyribonucleotide of embodiment [31] or embodiment [32], wherein the second coronavirus immunogen comprises an amino acid sequence of any one of SEQ ID NOs: 1-10, 53, 55, 57, 63-111, and 283-291.
[34] A circular polyribonucleotide comprising a first sequence encoding a coronavirus immunogen and a second sequence encoding a multimerization domain.
[35] The circular polyribonucleotide of embodiment [34], wherein the multimerization domain comprises a T4 foldon domain.
[36] The circular polyribonucleotide of embodiment [34], wherein the multimerization domain comprises a ferritin domain.
[37] The circular polyribonucleotide of embodiment [34], wherein the multimerization domain comprises a β-annulus peptide.
[38] The circular polyribonucleotide of any one of embodiments [34]-[37], wherein the multimerization domain is at the N-terminus of the coronavirus immunogen.
[39] The circular polyribonucleotide of any one of embodiments [34]-[37], wherein the multimerization domain is at the C-terminus of the coronavirus immunogen.
[40] An immunogenic composition comprising the circular polyribonucleotide of any one of embodiments [1]-[39] and a pharmaceutically acceptable excipient and is free of any carrier.
[41] An immunogenic composition comprising the circular polyribonucleotide of any one of embodiments [1]-[39] and a pharmaceutically acceptable carrier or excipient.
[42] The immunogenic composition of embodiment [40] or embodiment [41], wherein the composition further comprises a second circular polyribonucleotide.
[43] The immunogenic composition of embodiment [42], wherein the second circular polyribonucleotide comprises and open reading frame encoding a second polypeptide immunogen.
[44] The immunogenic composition of embodiment [42], wherein the second circular polyribonucleotide comprises an open reading frame encoding a polypeptide adjuvant.
[45] The immunogenic composition of embodiment [42], wherein the second circular polyribonucleotide comprises a non-coding ribonucleic acid sequence that is an innate immune system stimulator.
[46]A linear polyribonucleotide comprising an open reading frame encoding a coronavirus immunogen, wherein the coronavirus immunogen comprises an amino acid sequence having at least 95% sequence identity with the amino acid sequence of any one of SEQ ID NOs: 63-111 and 283-291.
[47] The linear polyribonucleotide of embodiment [46], wherein the coronavirus immunogen comprises an amino acid sequence of any one of SEQ ID NOs: 63-111 and 283-291.
[48] The linear polyribonucleotide of embodiment [46] or embodiment [47], wherein the open reading frame comprises a nucleic acid sequence having at least 95% sequence identity with the nucleic acid sequence of any one of SEQ ID NOs: 112-174 and 292-300.
[49] The linear polyribonucleotide of embodiment [46], wherein the open reading frame comprises a nucleic acid sequence of any one of SEQ ID NOs: 112-174 and 292-300.
[50] The linear polyribonucleotide of any one of embodiments [46]-[49], wherein the open reading frame encoding the coronavirus immunogen is operably linked to an IRES.
[51] The linear polyribonucleotide of any one of embodiments [46]-[50], wherein the open reading frame encoding the coronavirus immunogen encodes a second polypeptide.
[52] The linear polyribonucleotide of embodiment [51], wherein the coronavirus immunogen and the second polypeptide are separated by a polypeptide linker, a 2A self-cleaving peptide, a protease cleavage site, or 2A self-cleaving peptide in tandem with a protease cleavage site.
[53] The linear polyribonucleotide of embodiment [52], wherein the protease cleavage site is a furin cleavage site.
[54] The linear polyribonucleotide of any one of embodiments [46]-[49], wherein the circular polyribonucleotide further comprises a second open reading frame encoding a second polypeptide operably linked to a second IRES.
[55] The linear polyribonucleotide of any one of embodiments [51]-[54], wherein the second polypeptide is a polypeptide immunogen.
[56] The linear polyribonucleotide of embodiment [55], wherein the second polypeptide is a coronavirus immunogen.
[57] The linear polyribonucleotide of any one of embodiments [51]-[54], wherein the second polypeptide is a polypeptide adjuvant.
[58] The linear polyribonucleotide of embodiment [57], wherein the adjuvant is a cytokine, a chemokine, a costimulatory molecule, an innate immune stimulator, a signaling molecule, a transcriptional activator, a cytokine receptor, a bacterial component, or a component of the innate immune system.
[59] The linear polyribonucleotide of any one of embodiments [46]-[58], wherein the linear polyribonucleotide further comprises a non-coding ribonucleic acid sequence that is an innate immune system stimulator.
[60] The linear polyribonucleotide of embodiment [59], wherein the innate immune system stimulator is selected from a GU-rich motif, an AU-rich motif, a structured region comprising dsRNA, or an aptamer.
[61]A linear polyribonucleotide comprising a first sequence encoding a coronavirus immunogen and a second sequence encoding a multimerization domain.
[62] The linear polyribonucleotide of embodiment [61], wherein the multimerization domain comprises a T4 foldon domain.
[63] The linear polyribonucleotide of embodiment [61], wherein the multimerization domain comprises a ferritin domain.
[64] The linear polyribonucleotide of embodiment [61], wherein the multimerization domain comprises a pi-annulus peptide.
[65] The linear polyribonucleotide of any one of embodiments [61]-[64], wherein the multimerization domain is at the N-terminus of the coronavirus immunogen.
[66] The linear polyribonucleotide of any one of embodiments [61]-[64], wherein the multimerization domain is at the C-terminus of the coronavirus immunogen.
[67] An immunogenic composition comprising the linear polyribonucleotide of any one of embodiments [46]-[66] and a pharmaceutically acceptable excipient and is free of any carrier.
[68] An immunogenic composition comprising the linear polyribonucleotide of any one of embodiments [46]-[66] and a pharmaceutically acceptable carrier or excipient.
[69] The immunogenic composition of embodiment [67] or embodiment [68], wherein the composition further comprises a second linear polyribonucleotide.
[70] The immunogenic composition of embodiment [69], wherein the second linear polyribonucleotide comprises an open reading frame encoding a second polypeptide immunogen.
[71] The immunogenic composition of embodiment [69], wherein the second linear polyribonucleotide comprises an open reading frame encoding a polypeptide adjuvant.
[72] The immunogenic composition of embodiment [69], wherein the second linear polyribonucleotide comprises a non-coding ribonucleic acid sequence that is an innate immune system stimulator.
[73]A method of inducing an immune response against a coronavirus immunogen in a non-human animal or human subject comprising a) administering the immunogenic composition of any one of embodiments [40]-[45] and [67]-[72] to the non-human animal or human subject, and b) collecting antibodies against the coronavirus immunogen from the non-human animal or human subject.
[74] The method of embodiment [73], further comprising administering an adjuvant to the non-human animal or human subject.
[75]A method of inducing an immune response in a subject against SARS-CoV-2, the method comprising administering to the subject the circular polyribonucleotide, linear polyribonucleotide, or immunogenic compositions of any one of embodiments [1]-[72].
[76]. A method of treating a subject who has or is suspected to have a SARS-CoV-2 infection, the method comprising administering to the subject the circular polyribonucleotide or immunogenic composition of any one of embodiments [1]-[72].
[77]A method of preventing a SARS-CoV-2 infection in a subject, the method comprising administering to the subject the circular polyribonucleotide or immunogenic composition of any one of embodiments [1]-[72].
[78] The method of embodiment [77], wherein the human subject is at risk for a SARS-CoV-2 infection.
[79] The method of embodiment [76] or [78], wherein the human subject is a human over 50 years old, an immune-compromised human, a human with a chronic health condition, or a health care worker.
[80] The method of any one of embodiments [77]-[79], wherein administering the circular polyribonucleotide or immunogenic composition decreases the frequency or severity of symptoms associated with a SARS-CoV-2 infection.
[81] The method of any one of embodiments [76]-[82], wherein the subject is a human subject.
[82] The method of any one of embodiments [76]-[82], further comprising administering an adjuvant to the subject.
[83] The method of any one of embodiments [76]-[82], further comprising administering a SARS-CoV-2 immunogen to the subject.

SEQUENCE LISTING

| SEQ | | |
|---|---|---|
| ID | | |
| NO: | Comment | Sequence |
| 11 | QHD42416.1 | atgtttgtttttcttgttttattgccactagtctctagtcagtgtgttaatcttacaaccagaactcaattaccccctgcatacactaattctttcacacgtggtgtttattaccctgaca |
| | | aagttttcagatcctcagttttacattcaactcaggacttgttcttacctttcttttccaatgttacttggttccatgctatacatgtctctgggaccaatggtactaagaggtttgataa |
| | | ccctgtcctaccatttaatgatggtgtttattttgcttccactgagaagtctaacataataagaggctggatttttggtactactttagattcgaagacccagtccctacttattgttaat |
| | | aacgctactaatgttgttattaaagtctgtgaatttcaattttgtaatgatccatttttgggtgtttattaccacaaaaacaacaaaagttggatggaaagtgagttcagagtttattcta |
| | | gtgcgaataattgcacttttgaatatgtctctcagccttttcttatggaccttgaaggaaaacagggtaatttcaaaaatcttagggaatttgtgtttaagaatattgatggttattttaa |
| | | aatatattctaagcacacgcctattaatttagtgcgtgatctccctcagggtttttcggctttagaaccattggtagatttgccaataggtattaacatcactaggtttcaaactttactt |
| | | gctttacatagaagttatttgactcctggtgattcttcttcaggttggacagctggtgctgcagcttattatgtgggttatcttcaacctaggacttttctattaaaatataatgaaaatg |
| | | gaaccattacagatgctgtagactgtgcacttgaccctctctcagaaacaaagtgtacgttgaaatccttcactgtagaaaaaggaatctatcaaacttctaactttagagtccaaccaac |
| | | agaatctattgttagatttcctaatattacaaacttgtgcccttttggtgaagtttttaacgccaccagatttgcatctgtttatgcttggaacaggaagagaatcagcaactgtgttgct |
| | | gattattctgtcctatataattccgcatcattttccacttttaagtgttatggagtgtctcctactaaattaaatgatctctgctttactaatgtctatgcagattcatttgtaattagag |
| | | gtgatgaagtcagacaaatcgctccagggcaaactggaaagattgctgattataattataaattaccagatgattttacaggctgcgttatagcttggaattctaacaatcttgattctaa |
| | | ggttggtggtaattataattacctgtatagattgtttaggaagtctaatctcaaaccttttgagagagatatttcaactgaaatctatcaggccggtagcacaccttgtaatggtgttgaa |
| | | ggttttaattgttactttcctttacaatcatatggtttccaacccactaatggtgttggttaccaaccatacagagtagtagtactttcttttgaacttctacatgcaccagcaactgttt |
| | | gtggacctaaaaagtctactaatttggttaaaaacaaatgtgtcaatttcaacttcaatggtttaacaggcacaggtgttcttactgagtctaacaaaaagtttctgcctttccaacaatt |
| | | tggcagagacattgctgacactactgatgctgtccgtgatccacagacacttgagattcttgacattacaccatgttcttttggtggtgtcagtgttataacaccaggaacaaatacttct |
| | | aaccaggttgctgttctttatcaggatgttaactgcacagaagtccctgttgctattcatgcagatcaacttactcctacttggcgtgtttattctacaggttctaatgtttttcaaacac |
| | | gtgcaggctgtttaataggggctgaacatgtcaacaactcatatgagtgtgacatacccattggtgcaggtatatgcgctagttatcagactcagactaattctcctcggcgggca |
| | | cgtagtgtagctagtcaatccatcattgcctacactatgtcacttggtgcagaaaattcagttgcttactctaataactctattgccatacccacaaattttactattagtgttaccaca |
| | | gaaattctaccagtgtctatgaccaagacatcagtagattgtacaatgtacatttgtggtgattcaactgaatgcagcaatcttttgttgcaatatggcagtttttgtacacaattaaa |
| | | ccgtgctttaactggaatagctgttgaacaagacaaaaacacccaagaagtttttgcacaagtcaaacaaatttacaaaacaccaccaattaaagattttggtggttttaattttt |
| | | cacaaatattaccagatccatcaaaaccaagcaagaggtcatttattgaagatctacttttcaacaaagtgacacttgcagatgctggcttcatcaaacaatatggtgattgcctt |
| | | ggtgatattgctgctagagacctcatttgtgcacaaaagtttaacggccttactgttttgccacctttgctcacagatgaaatgattgctcaatacacttctgcactgttagcgggtac |
| | | aatcacttctggttggacctttggtgcaggtgctgcattacaaataccatttgctatgcaaatggcttataggtttaatggtattggagttacacagaatgttctctatgagaaccaaa |
| | | aattgattgccaaccaatttaatagtgctattggcaaaattcaagactcactttcttccacagcaagtgcacttggaaaacttcaagatgtggtcaaccaaaatgcacaagcttta |
| | | aacacgcttgttaaacaacttagctccaattttggtgcaatttcaagtgttttaaatgatatcctttcacgtcttgacaaagttgaggctgaagtgcaaattgataggttgatcacagg |
| | | cagacttcaaagtttgcagacatatgtgactcaacaattaattagagctgcagaaatcagagcttctgctaatcttgctgctactaaaatgtcagagtgtgtacttggacaatcaa |
| | | aaagagttgatttttgtggaaagggctatcatcttatgtccttccctcagtcagcacctcatggtgtagtcttcttgcatgtgacttatgtccctgcacaagaaaagaacttcacaact |
| | | gctcctgccatttgtcatgatggaaaagcacactttcctcgtgaaggtgtctttgtttcaaatggcacacactggtttgtaacacaaaggaatttttatgaaccacaaatcattacta |
| | | cagacaacacatttgtgtctggtaactgtgatgttgtaataggaattgtcaacaacacagtttatgatcctttgcaacctgaattagactcattcaaggaggagttagataaatattt |
| | | taagaatcatacatcaccagatgttgatttaggtgacatctctggcattaatgcttcagttgtaaacattcaaaaagaaattgaccgcctcaatgaggttgccaagaatttaaatg |
| | | aatctctcatcgatctccaagaacttggaaagtatgagcagtatataaaatggccatggtacatttggctaggttttatagctggcttgattgccatagtaatggtgacaattatgctt |
| | | tgctgtatgaccagttgctgtagttgtctcaagggctgttgttcttgtggatcctgctgcaaatttgatgaagacgactctgagccagtgctcaaaggagtcaaattacattacacat |
| | | aa |
| 12 | p1 | AGATTATCAAAAAGGATCTTCACCTAGATCCTTTTAAATTAAAAATGAAGTTTTAAATCAATCTAAAGTATATATGAGTAA |
| | | ACTTGGTCTGACAGTTATTAGAAAAATTCATCCAGCAGACGATAAAACGCAATACGCTGGCTATCCGGTGCCGCAATG |
| | | CCATACAGCACCAGAAAACGATCCGCCCATTCGCCGCCCAGTTCTTCCGCAATATCACGGGTGGCCAGCGCAATATC |
| | | CTGATAACGATCCGCCACGCCCAGACGGCCGCAATCAATAAAGCCGCTAAAACGGCCATTTTCCACCATAATGTTCG |
| | | GCAGGCACGCATCACCATGGGTCACCACCAGATCTTCGCCATCCGGCATGCTCGCTTTCAGACGCGCAAACAGCTCT |
| | | GCCGGTGCCAGGCCCTGATGTTCTTCATCCAGATCATCCTGATCCACCAGGCCCGCTTCCATACGGGTACGCGCACG |
| | | TTCAATACGATGTTTCGCCTGATGATCAAACGGACAGGTCGCCGGGTCCAGGGTATGCAGACGACGCATGGCATCCG |
| | | CCATAATGCTCACTTTTTCTGCCGGCGCCAGATGGCTAGACAGCAGATCCTGACCCGGCACTTCGCCCAGCAGCAGC |
| | | CAATCACGGCCCGCTTCGGTCACCACATCCAGCACCGCCGCACACGGAACACCGGTGGTGGCCAGCCAGCTCAGAC |
| | | GCGCCGCTTCATCCTGCAGCTCGTTCAGCGCACCGCTCAGATCGGTTTTCACAAACAGCACCGGACGACCCTGCGC |
| | | GCTCAGACGAAACACCGCCGCATCAGAGCAGCCAATGGTCTGCTGCGCCCAATCATAGCCAAACAGACGTTCCACCC |
| | | ACGCTGCCGGGCTACCCGCATGCAGGCCATCCTGTTCAATCATACTCTTCCTTTTTCAATATTATTGAAGCATTTATCA |
| | | GGGTTATTGTCTCATGAGCGGATACATATTTGAATGTATTTAGAAAAATAAACAAATAGGGGTTCCGCGCACATTTCCC |
| | | CGAAAAGTGCCACCTAAATTGTAAGCGTTAATATTTTGTTAAAATTCGCGTTAAATTTTTGTTAAATCAGCTCATTTTTTA |
| | | ACCAATAGGCCGAAATCGGCAAAATCCCTTATAAATCAAAAGAATAGACCGAGATAGGGTTGAGTGGCCGCTACAGG |
| | | GCGCTCCCATTCGCCATTCAGGCTGCGCAACTGTTGGGAAGGGCGTTTCGGTGCGGGCCTCTTCGCTATTACGCCA |
| | | GCTGGCGAAAGGGGGATGTGCTGCAAGGCGATTAAGTTGGGTAACGCCAGGGTTTTCCCAGTCACGACGTTGTAAAA |
| | | CGACGGCCAGTGAGCGCGACGTAATACGACTCACTATAGGGCGAATTGAAGGAAGGCCGTCAAGGCCGCATGGGAA |
| | | GCCCTCGACCGTCGATTGTCCACTGGTCAACAATAGATGACTTACAACTAATCGGAAGGTGCAGAGACTCGACGGGA |
| | | GCTACCCTAACGTCAAGACGAGGGTAAAGAGAGAGTCCAATTCTCAAAGCCAATAGGCAGTAGCGAAAGCTGCAAGA |
| | | GAATGAAAATCCGTTGACCTTAAACGGTCGTGTGGGTTCAAGTCCCTCCACCCCCACGCCGGAAACGCAATAGCCGA |
| | | AAAACAAAAAACAAAAAAAACAAAAAAAAAACCAAAAAAACAAAACACAacgttactggccgaagccgcttggaataaggccggtgtgcgttt |
| | | gtctatatgttattttccaccatattgccgtcttttggcaatgtgagggcccggaaacctggccctgtcttcttgacgagcattcctaggggtctttcccctctcgccaaaggaatgca |
| | | aggtctgttgaatgtcgtgaaggaagcagttcctctggaagcttcttgaagacaaacaacgtctgtagcgaccctttgcaggcagcggaaccccccacctggcgacaggtgc |
| | | ctctgcggccaaaagccacgtgtataagatacacctgcaaaggcggcacaaccccagtgccacgttgtgagttggatagttgtggaaagagtcaaatggctctcctcaagc |
| | | gtattcaacaaggggctgaaggatgcccagaaggtaccccattgtatgggatctgatctggggcctcggtgcacatgctttacatgtgtttagtcgaggttaaaaaacgtctag |
| | | gccccccgaaccacggggacgtggttttcctttgaaaaacacgatgataataGCCACCatgtttgtttttcttgttttattgccactagtctctagtcagtgtgttaatcttacaacc |
| | | agaactcaattaccccctgcatacactaattctttcacacgtggtgtttattaccctgacaaagttttcagatcctcagttttacattcaactcaggacttgttcttacctttcttttcca |
| | | atgttacttggttccatgctatacatgtctctgggaccaatggtactaagaggtttgataaccctgtcctaccatttaatgatggtgtttattttgcttccactgagaagtctaacataat |
| | | aagaggctggatttttggtactactttagattcgaagacccagtccctacttattgttaataacgctactaatgttgttattaaagtctgtgaatttcaattttgtaatgatccatttttg |
| | | ggtgtttattaccacaaaaacaacaaaagttggatggaaagtgagttcagagtttattctagtgcgaataattgcacttttgaatatgtctctcagccttttcttatggaccttgaaggaaa |
| | | acagggtaatttcaaaaatcttagggaatttgtgtttaagaatattgatggttattttaaaatatattctaagcacacgcctattaatttagtgcgtgatctccctcagggtttttcggctt |
| | | tagaaccattggtagatttgccaataggtattaacatcactaggtttcaaactttacttgctttacatagaagttatttgactcctggtgattcttcttcaggttggacagctggtgctgca |
| | | gcttattatgtgggttatcttcaacctaggacttttctattaaaatataatgaaaatggaaccattacagatgctgtagactgtgcacttgaccctctctcagaaacaaagtgtacgttga |
| | | aatccttcactgtagaaaaaggaatctatcaaacttctaactttagagtccaaccaacagaatctattgttagatttcctaatattacaaacttgtgcccttttggtgaagtttttaacg |
| | | ccaccagatttgcatctgtttatgcttggaacaggaagagaatcagcaactgtgttgctgattattctgtcctatataattccgcatcattttccacttttaagtgttatggagtgtctcct |
| | | actaaattaaatgatctctgctttactaatgtctatgcagattcatttgtaattagaggtgatgaagtcagacaaatcgctccagggcaaactggaaagattgctgattataattata |
| | | aattaccagatgattttacaggctgcgttatagcttggaattctaacaatcttgattctaaggttggtggtaattataattacctgtatagattgtttaggaagtctaatctcaaacctttt |
| | | gagagagatatttcaactgaaatctatcaggccggtagcacaccttgtaatggtgttgaaggttttaattgttactttcctttacaatcatatggtttccaacccactaatggtgttggtt |
| | | accaaccatacagagtagtagtactttcttttgaacttctacatgcaccagcaactgtttgtggacctaaaaagtctactaatttggttaaaaacaaatgtgtcaatttcaacttcaat |
| | | ggtttaacaggcacaggtgttcttactgagtctaacaaaaagtttctgcctttccaacaatttggcagagacattgctgacactactgatgctgtccgtgatccacagacacttga |
| | | gattcttgacattacaccatgttcttttggtggtgtcagtgttataacaccaggaacaaatacttctaaccaggttgctgttctttatcaggatgttaactgcacagaagtccctgttgct |
| | | attcatgcagatcaacttactcctacttggcgtgtttattctacaggttctaatgtttttcaaacacgtgcaggctgtttaataggggctgaacatgtcaacaactcatatgagtgtgac |
| | | atacccattggtgcaggtatatgcgctagttatcagactcagactaattctcctggcagcgccagcagtgtagctagtcaatccatcattgcctacactatgtcacttggtgcaga |
| | | aaattcagttgcttactctaataactctattgccatacccacaaattttactattagtgttaccacagaaattctaccagtgtctatgaccaagacatcagtagattgtacaatgtaca |
| | | tttgtggtgattcaactgaatgcagcaatcttttgttgcaatatggcagtttttgtacacaattaaaccgtgctttaactggaatagctgttgaacaagacaaaaacacccaagaag |
| | | tttttgcacaagtcaaacaaatttacaaaacaccaccaattaaagattttggtggttttaatttttcacaaatattaccagatccatcaaaaccaagcaagaggtcatttattgaag |
| | | atctacttttcaacaaagtgacacttgcagatgctggcttcatcaaacaatatggtgattgccttggtgatattgctgctagggacctcatttgtgcacaaaagtttaacggccttact |
| | | gttttgccacctttgctcacagatgaaatgattgctcaatacacttctgcactgttagcgggtacaatcacttctggttggacctttggtgcaggtgctgcattacaaataccatttgct |
| | | atgcaaatggcttataggtttaatggtattggagttacacagaatgttctctatgagaaccaaaaattgattgccaaccaatttaatagtgctattggcaaaattcaagactcacttt |
| | | cttccacagcaagtgcacttggaaaacttcaagatgtggtcaaccaaaatgcacaagctttaaacacgcttgttaaacaacttagctccaattttggtgcaatttcaagtgttttaa |
| | | atgatatcctttcacgtcttgaccctcccgaggctgaagtgcaaattgataggttgatcacaggcagacttcaaagtttgcagacatatgtgactcaacaattaattagagctgca |
| | | gaaatcagagcttctgctaatcttgctgctactaaaatgtcagagtgtgtacttggacaatcaaaaagagttgatttttgtggaaagggctatcatcttatgtccttccctcagtcag |
| | | cacctcatggtgtagtcttcttgcatgtgacttatgtccctgcacaagaaaagaacttcacaactgctcctgccatttgtcatgatggaaaagcacactttcctcgtgaaggtgtcttt |
| | | gtttcaaatggcacacactggtttgtaacacaaaggaatttttatgaaccacaaatcattactacagacaacacatttgtgtctggtaactgtgatgttgtaataggaattgtcaac |
| | | aacacagtttatgatcctttgcaacctgaattagactcattcaaggaggagttagataaatattttaagaatcatacatcaccagatgttgatttaggtgacatctctggcattaatg |
| | | cttcagttgtaaacattcaaaaagaaattgaccgcctcaatgaggttgccaagaatttaaatgaatctctcatcgatctccaagaacttggaaagtatgagcagGGGTATA |
| | | TCCCTGAAGCCCCCAGGGACGGCCAGGCTTACGTCAGAAAGGATGGAGAGTGGGTGCTCTTGAGCACCTTCCTGTA |
| | | AAAAAAACAAAAAACAAAACGGCTATTATGCGTTACCGGCGAGACGCTACGGACTTAAATAATTGAGCCTTAAAGAAG |
| | | AAATTCTTTAAGTGGATGCTCTCAAACTCAGGGAAACCTAAATCTAGTTATAGACAAGGCAATCCTGAGCCAAGCCGA |
| | | AGTAGTAATTAGTAAGACCAGTGGACAATCGACGGATAACAGCATATCTAGCTGGGCCTCATGGGCCTTCCTTTCACT |
| | | GCCCGCTTTCCAGTCGGGAAACCTGTCGTGCCAGCTGCATTAACATGGTCATAGCTGTTTCCTTGCGTATTGGGCGC |
| | | TCTCCGCTTCCTCGCTCACTGACTCGCTGCGCTCGGTCGTTCGGGTAAAGCCTGGGGTGCCTAATGAGCAAAAGGCC |
| | | AGCAAAAGGCCAGGAACCGTAAAAAGGCCGCGTTGCTGGCGTTTTTCCATAGGCTCCGCCCCCCTGACGAGCATCAC |
| | | AAAAATCGACGCTCAAGTCAGAGGTGGCGAAACCCGACAGGACTATAAAGATACCAGGCGTTTCCCCCTGGAAGCTC |
| | | CCTCGTGCGCTCTCCTGTTCCGACCCTGCCGCTTACCGGATACCTGTCCGCCTTTCTCCCTTCGGGAAGCGTGGCGC |
| | | TTTCTCATAGCTCACGCTGTAGGTATCTCAGTTCGGTGTAGGTCGTTCGCTCCAAGCTGGGCTGTGTGCACGAACCC |
| | | CCCGTTCAGCCCGACCGCTGCGCCTTATCCGGTAACTATCGTCTTGAGTCCAACCCGGTAAGACACGACTTATCGCC |
| | | ACTGGCAGCAGCCACTGGTAACAGGATTAGCAGAGCGAGGTATGTAGGCGGTGCTACAGAGTTCTTGAAGTGGTGG |
| | | CCTAACTACGGCTACACTAGAAGAACAGTATTTGGTATCTGCGCTCTGCTGAAGCCAGTTACCTTCGGAAAAAGAGTT |
| | | GGTAGCTCTTGATCCGGCAAACAAACCACCGCTGGTAGCGGTGGTTTTTTTGTTTGCAAGCAGCAGATTACGCGCAG |
| | | AAAAAAAGGATCTCAAGAAGATCCTTTGATCTTTTCTACGGGGTCTGACGCTCAGTGGAACGAAAACTCACGTTAAGG |
| | | GATTTTGGTCATG |
| 13 | ORF1 | atgtttgtttttcttgttttattgccactagtctctagtcagtgtgttaatcttacaaccagaactcaattaccccctgcatacactaattctttcacacgtggtgtttattaccctgacaa |
| | | agttttcagatcctcagttttacattcaactcaggacttgttcttacctttcttttccaatgttacttggttccatgctatacatgtctctgggaccaatggtactaagaggtttgataacc |
| | | ctgtcctaccatttaatgatggtgtttattttgcttccactgagaagtctaacataataagaggctggatttttggtactactttagattcgaagacccagtccctacttattgttaataac |
| | | gctactaatgttgttattaaagtctgtgaatttcaattttgtaatgatccatttttgggtgtttattaccacaaaaacaacaaaagttggatggaaagtgagttcagagtttattctagtgc |
| | | gaataattgcacttttgaatatgtctctcagccttttcttatggaccttgaaggaaaacagggtaatttcaaaaatcttagggaatttgtgtttaagaatattgatggttattttaaaatat |
| | | attctaagcacacgcctattaatttagtgcgtgatctccctcagggtttttcggctttagaaccattggtagatttgccaataggtattaacatcactaggtttcaaactttacttgcttta |
| | | catagaagttatttgactcctggtgattcttcttcaggttggacagctggtgctgcagcttattatgtgggttatcttcaacctaggacttttctattaaaatataatgaaaatggaaccat |
| | | tacagatgctgtagactgtgcacttgaccctctctcagaaacaaagtgtacgttgaaatccttcactgtagaaaaaggaatctatcaaacttctaactttagagtccaaccaacaga |
| | | atctattgttagatttcctaatattacaaacttgtgcccttttggtgaagtttttaacgccaccagatttgcatctgtttatgcttggaacaggaagagaatcagcaactgtgttgctgatt |
| | | attctgtcctatataattccgcatcattttccacttttaagtgttatggagtgtctcctactaaattaaatgatctctgctttactaatgtctatgcagattcatttgtaattagaggtgat |
| | | gaagtcagacaaatcgctccagggcaaactggaaagattgctgattataattataaattaccagatgattttacaggctgcgttatagcttggaattctaacaatcttgattctaaggttg |
| | | gtggtaattataattacctgtatagattgtttaggaagtctaatctcaaaccttttgagagagatatttcaactgaaatctatcaggccggtagcacaccttgtaatggtgttgaaggt |
| | | tttaattgttactttcctttacaatcatatggtttccaacccactaatggtgttggttaccaaccatacagagtagtagtactttcttttgaacttctacatgcaccagcaactgtttgtgg |
| | | acctaaaaagtctactaatttggttaaaaacaaatgtgtcaatttcaacttcaatggtttaacaggcacaggtgttcttactgagtctaacaaaaagtttctgcctttccaacaatttgg |
| | | cagagacattgctgacactactgatgctgtccgtgatccacagacacttgagattcttgacattacaccatgttcttttggtggtgtcagtgttataacaccaggaacaaatacttct |
| | | aaccaggttgctgttctttatcaggatgttaactgcacagaagtccctgttgctattcatgcagatcaacttactcctacttggcgtgtttattctacaggttctaatgtttttcaaacacg |
| | | tgcaggctgtttaataggggctgaacatgtcaacaactcatatgagtgtgacatacccattggtgcaggtatatgcgctagttatcagactcagactaattctcctggcagcgcc |
| | | agcagtgtagctagtcaatccatcattgcctacactatgtcacttggtgcagaaaattcagttgcttactctaataactctattgccatacccacaaattttactattagtgttaccaca |
| | | gaaattctaccagtgtctatgaccaagacatcagtagattgtacaatgtacatttgtggtgattcaactgaatgcagcaatcttttgttgcaatatggcagtttttgtacacaattaaa |
| | | ccgtgctttaactggaatagctgttgaacaagacaaaaacacccaagaagtttttgcacaagtcaaacaaatttacaaaacaccaccaattaaagattttggtggttttaattttt |
| | | cacaaatattaccagatccatcaaaaccaagcaagaggtcatttattgaagatctacttttcaacaaagtgacacttgcagatgctggcttcatcaaacaatatggtgattgcctt |
| | | ggtgatattgctgctagggacctcatttgtgcacaaaagtttaacggccttactgttttgccacctttgctcacagatgaaatgattgctcaatacacttctgcactgttagcgggtac |
| | | aatcacttctggttggacctttggtgcaggtgctgcattacaaataccatttgctatgcaaatggcttataggtttaatggtattggagttacacagaatgttctctatgagaaccaaa |
| | | aattgattgccaaccaatttaatagtgctattggcaaaattcaagactcactttcttccacagcaagtgcacttggaaaacttcaagatgtggtcaaccaaaatgcacaagcttta |
| | | aacacgcttgttaaacaacttagctccaattttggtgcaatttcaagtgttttaaatgatatcctttcacgtcttgaccctcccgaggctgaagtgcaaattgataggttgatcacagg |
| | | cagacttcaaagtttgcagacatatgtgactcaacaattaattagagctgcagaaatcagagcttctgctaatcttgctgctactaaaatgtcagagtgtgtacttggacaatcaa |
| | | aaagagttgatttttgtggaaagggctatcatcttatgtccttccctcagtcagcacctcatggtgtagtcttcttgcatgtgacttatgtccctgcacaagaaaagaacttcacaact |
| | | gctcctgccatttgtcatgatggaaaagcacactttcctcgtgaaggtgtctttgtttcaaatggcacacactggtttgtaacacaaaggaatttttatgaaccacaaatcattacta |
| | | cagacaacacatttgtgtctggtaactgtgatgttgtaataggaattgtcaacaacacagtttatgatcctttgcaacctgaattagactcattcaaggaggagttagataaatattt |
| | | taagaatcatacatcaccagatgttgatttaggtgacatctctggcattaatgcttcagttgtaaacattcaaaaagaaattgaccgcctcaatgaggttgccaagaatttaaatg |
| | | aatctctcatcgatctccaagaacttggaaagtatgagcagGGGTATATCCCTGAAGCCCCCAGGGACGGCCAGGCTTACGTCAGAAAG |
| | | GATGGAGAGTGGGTGCTCTTGAGCACCTTCCTGTAA |
| 14 | p3 | AGATTATCAAAAAGGATCTTCACCTAGATCCTTTTAAATTAAAAATGAAGTTTTAAATCAATCTAAAGTATATATGAGTAA |
| | | ACTTGGTCTGACAGTTATTAGAAAAATTCATCCAGCAGACGATAAAACGCAATACGCTGGCTATCCGGTGCCGCAATG |
| | | CCATACAGCACCAGAAAACGATCCGCCCATTCGCCGCCCAGTTCTTCCGCAATATCACGGGTGGCCAGCGCAATATC |
| | | CTGATAACGATCCGCCACGCCCAGACGGCCGCAATCAATAAAGCCGCTAAAACGGCCATTTTCCACCATAATGTTCG |
| | | GCAGGCACGCATCACCATGGGTCACCACCAGATCTTCGCCATCCGGCATGCTCGCTTTCAGACGCGCAAACAGCTCT |
| | | GCCGGTGCCAGGCCCTGATGTTCTTCATCCAGATCATCCTGATCCACCAGGCCCGCTTCCATACGGGTACGCGCACG |
| | | TTCAATACGATGTTTCGCCTGATGATCAAACGGACAGGTCGCCGGGTCCAGGGTATGCAGACGACGCATGGCATCCG |
| | | CCATAATGCTCACTTTTTCTGCCGGCGCCAGATGGCTAGACAGCAGATCCTGACCCGGCACTTCGCCCAGCAGCAGC |
| | | CAATCACGGCCCGCTTCGGTCACCACATCCAGCACCGCCGCACACGGAACACCGGTGGTGGCCAGCCAGCTCAGAC |
| | | GCGCCGCTTCATCCTGCAGCTCGTTCAGCGCACCGCTCAGATCGGTTTTCACAAACAGCACCGGACGACCCTGCGC |
| | | GCTCAGACGAAACACCGCCGCATCAGAGCAGCCAATGGTCTGCTGCGCCCAATCATAGCCAAACAGACGTTCCACCC |
| | | ACGCTGCCGGGCTACCCGCATGCAGGCCATCCTGTTCAATCATACTCTTCCTTTTTCAATATTATTGAAGCATTTATCA |
| | | GGGTTATTGTCTCATGAGCGGATACATATTTGAATGTATTTAGAAAAATAAACAAATAGGGGTTCCGCGCACATTTCCC |
| | | CGAAAAGTGCCACCTAAATTGTAAGCGTTAATATTTTGTTAAAATTCGCGTTAAATTTTTGTTAAATCAGCTCATTTTTTA |
| | | ACCAATAGGCCGAAATCGGCAAAATCCCTTATAAATCAAAAGAATAGACCGAGATAGGGTTGAGTGGCCGCTACAGG |
| | | GCGCTCCCATTCGCCATTCAGGCTGCGCAACTGTTGGGAAGGGCGTTTCGGTGCGGGCCTCTTCGCTATTACGCCA |
| | | GCTGGCGAAAGGGGGATGTGCTGCAAGGCGATTAAGTTGGGTAACGCCAGGGTTTTCCCAGTCACGACGTTGTAAAA |
| | | CGACGGCCAGTGAGCGCGACGTAATACGACTCACTATAGGGCGAATTGAAGGAAGGCCGTCAAGGCCGCATGGGAA |
| | | GCCCTCGACCGTCGATTGTCCACTGGTCAACAATAGATGACTTACAACTAATCGGAAGGTGCAGAGACTCGACGGGA |
| | | GCTACCCTAACGTCAAGACGAGGGTAAAGAGAGAGTCCAATTCTCAAAGCCAATAGGCAGTAGCGAAAGCTGCAAGA |
| | | GAATGAAAATCCGTTGACCTTAAACGGTCGTGTGGGTTCAAGTCCCTCCACCCCCACGCCGGAAACGCAATAGCCGA |
| | | AAAACAAAAAACAAAAAAAACAAAAAAAAAACCAAAAAAACAAAACACAacgttactggccgaagccgcttggaataaggccggtgtgcgttt |
| | | gtctatatgttattttccaccatattgccgtcttttggcaatgtgagggcccggaaacctggccctgtcttcttgacgagcattcctaggggtctttcccctctcgccaaaggaatgca |
| | | aggtctgttgaatgtcgtgaaggaagcagttcctctggaagcttcttgaagacaaacaacgtctgtagcgaccctttgcaggcagcggaaccccccacctggcgacaggtgc |
| | | ctctgcggccaaaagccacgtgtataagatacacctgcaaaggcggcacaaccccagtgccacgttgtgagttggatagttgtggaaagagtcaaatggctctcctcaagc |
| | | gtattcaacaaggggctgaaggatgcccagaaggtaccccattgtatgggatctgatctggggcctcggtgcacatgctttacatgtgtttagtcgaggttaaaaaacgtctag |
| | | gccccccgaaccacggggacgtggttttcctttgaaaaacacgatgataataGCCACCatgtttgtttttcttgttttattgccactagtctctagtcagtgtgttaatcttacaacc |
| | | agaactcaattaccccctgcatacactaattctttcacacgtggtgtttattaccctgacaaagttttcagatcctcagttttacattcaactcaggacttgttcttacctttcttttccaa |
| | | tgttacttggttccatgctatacatgtctctgggaccaatggtactaagaggtttgataaccctgtcctaccatttaatgatggtgtttattttgcttccactgagaagtctaacataataa |
| | | gaggctggatttttggtactactttagattcgaagacccagtccctacttattgttaataacgctactaatgttgttattaaagtctgtgaatttcaattttgtaatgatccatttttgggt |
| | | gtttattaccacaaaaacaacaaaagttggatggaaagtgagttcagagtttattctagtgcgaataattgcacttttgaatatgtctctcagccttttcttatggaccttgaaggaaaac |
| | | agggtaatttcaaaaatcttagggaatttgtgtttaagaatattgatggttattttaaaatatattctaagcacacgcctattaatttagtgcgtgatctccctcagggtttttcggcttta |
| | | gaaccattggtagatttgccaataggtattaacatcactaggtttcaaactttacttgctttacatagaagttatttgactcctggtgattcttcttcaggttggacagctggtgctgcagc |
| | | ttattatgtgggttatcttcaacctaggacttttctattaaaatataatgaaaatggaaccattacagatgctgtagactgtgcacttgaccctctctcagaaacaaagtgtacgttga |
| | | aatccttcactgtagaaaaaggaatctatcaaacttctaactttagagtccaaccaacagaatctattgttagatttcctaatattacaaacttgtgcccttttggtgaagtttttaacg |
| | | ccaccagatttgcatctgtttatgcttggaacaggaagagaatcagcaactgtgttgctgattattctgtcctatataattccgcatcattttccacttttaagtgttatggagtgtctcct |
| | | actaaattaaatgatctctgctttactaatgtctatgcagattcatttgtaattagaggtgatgaagtcagacaaatcgctccagggcaaactggaaagattgctgattataattata |
| | | aattaccagatgattttacaggctgcgttatagcttggaattctaacaatcttgattctaaggttggtggtaattataattacctgtatagattgtttaggaagtctaatctcaaacctttt |
| | | gagagagatatttcaactgaaatctatcaggccggtagcacaccttgtaatggtgttgaaggttttaattgttactttcctttacaatcatatggtttccaacccactaatggtgttggtt |
| | | accaaccatacagagtagtagtactttcttttgaacttctacatgcaccagcaactgtttgtggacctaaaaagtctactaatttggttaaaaacaaatgtgtcaatttcaacttcaat |
| | | ggtttaacaggcacaggtgttcttactgagtctaacaaaaagtttctgcctttccaacaatttggcagagacattgctgacactactgatgctgtccgtgatccacagacacttga |
| | | gattcttgacattacaccatgttcttttggtggtgtcagtgttataacaccaggaacaaatacttctaaccaggttgctgttctttatcaggatgttaactgcacagaagtccctgttgct |
| | | attcatgcagatcaacttactcctacttggcgtgtttattctacaggttctaatgtttttcaaacacgtgcaggctgtttaataggggctgaacatgtcaacaactcatatgagtgtgac |
| | | atacccattggtgcaggtatatgcgctagttatcagactcagactaattctcctggcagcgccagcagtgtagctagtcaatccatcattgcctacactatgtcacttggtgcaga |
| | | aaattcagttgcttactctaataactctattgccatacccacaaattttactattagtgttaccacagaaattctaccagtgtctatgaccaagacatcagtagattgtacaatgtaca |
| | | tttgtggtgattcaactgaatgcagcaatcttttgttgcaatatggcagtttttgtacacaattaaaccgtgctttaactggaatagctgttgaacaagacaaaaacacccaagaag |
| | | tttttgcacaagtcaaacaaatttacaaaacaccaccaattaaagattttggtggttttaatttttcacaaatattaccagatccatcaaaaccaagcaagaggtcatttattgaag |
| | | atctacttttcaacaaagtgacacttgcagatgctggcttcatcaaacaatatggtgattgccttggtgatattgctgctagggacctcatttgtgcacaaaagtttaacggccttact |
| | | gttttgccacctttgctcacagatgaaatgattgctcaatacacttctgcactgttagcgggtacaatcacttctggttggacctttggtgcaggtgctgcattacaaataccatttgct |
| | | atgcaaatggcttataggtttaatggtattggagttacacagaatgttctctatgagaaccaaaaattgattgccaaccaatttaatagtgctattggcaaaattcaagactcacttt |
| | | cttccacagcaagtgcacttggaaaacttcaagatgtggtcaaccaaaatgcacaagctttaaacacgcttgttaaacaacttagctccaattttggtgcaatttcaagtgttttaa |
| | | atgatatcctttcacgtcttgaccctcccgaggctgaagtgcaaattgataggttgatcacaggcagacttcaaagtttgcagacatatgtgactcaacaattaattagagctgca |
| | | gaaatcagagcttctgctaatcttgctgctactaaaatgtcagagtgtgtacttggacaatcaaaaagagttgatttttgtggaaagggctatcatcttatgtccttccctcagtcag |
| | | cacctcatggtgtagtcttcttgcatgtgacttatgtccctgcacaagaaaagaacttcacaactgctcctgccatttgtcatgatggaaaagcacactttcctcgtgaaggtgtcttt |
| | | gtttcaaatggcacacactggtttgtaacacaaaggaatttttatgaaccacaaatcattactacagacaacacatttgtgtctggtaactgtgatgttgtaataggaattgtcaac |
| | | aacacagtttatgatcctttgcaacctgaattagactcattcaaggaggagttagataaatattttaagaatcatacatcaccagatgttgatttaggtgacatctctggcattaatg |
| | | cttcagttgtaaacattcaaaaagaaattgaccgcctcaatgaggttgccaagaatttaaatgaatctctcatcgatctccaagaacttggaaagtatgagcagtatataaaatg |
| | | gccatggtacatttggctaggttttatagctggcttgattgccatagtaatggtgacaattatgctttgctgtatgaccagttgctgtagttgtctcaagggctgttgttcttgtggatcctg |
| | | ctgcaaatttgatgaagacgactctgagccagtgctcaaaggagtcaaattacattacacaTAAAAAAAACAAAAAACAAAACGGCTATTATGCGTT |
| | | ACCGGCGAGACGCTACGGACTTAAATAATTGAGCCTTAAAGAAGAAATTCTTTAAGTGGATGCTCTCAAACTCAGGGA |
| | | AACCTAAATCTAGTTATAGACAAGGCAATCCTGAGCCAAGCCGAAGTAGTAATTAGTAAGACCAGTGGACAATCGACG |
| | | GATAACAGCATATCTAGCTGGGCCTCATGGGCCTTCCTTTCACTGCCCGCTTTCCAGTCGGGAAACCTGTCGTGCCA |
| | | GCTGCATTAACATGGTCATAGCTGTTTCCTTGCGTATTGGGCGCTCTCCGCTTCCTCGCTCACTGACTCGCTGCGCTC |
| | | GGTCGTTCGGGTAAAGCCTGGGGTGCCTAATGAGCAAAAGGCCAGCAAAAGGCCAGGAACCGTAAAAAGGCCGCGT |
| | | TGCTGGCGTTTTTCCATAGGCTCCGCCCCCCTGACGAGCATCACAAAAATCGACGCTCAAGTCAGAGGTGGCGAAAC |
| | | CCGACAGGACTATAAAGATACCAGGCGTTTCCCCCTGGAAGCTCCCTCGTGCGCTCTCCTGTTCCGACCCTGCCGCT |
| | | TACCGGATACCTGTCCGCCTTTCTCCCTTCGGGAAGCGTGGCGCTTTCTCATAGCTCACGCTGTAGGTATCTCAGTTC |
| | | GGTGTAGGTCGTTCGCTCCAAGCTGGGCTGTGTGCACGAACCCCCCGTTCAGCCCGACCGCTGCGCCTTATCCGGT |
| | | AACTATCGTCTTGAGTCCAACCCGGTAAGACACGACTTATCGCCACTGGCAGCAGCCACTGGTAACAGGATTAGCAG |
| | | AGCGAGGTATGTAGGCGGTGCTACAGAGTTCTTGAAGTGGTGGCCTAACTACGGCTACACTAGAAGAACAGTATTTG |
| | | GTATCTGCGCTCTGCTGAAGCCAGTTACCTTCGGAAAAAGAGTTGGTAGCTCTTGATCCGGCAAACAAACCACCGCT |
| | | GGTAGCGGTGGTTTTTTTGTTTGCAAGCAGCAGATTACGCGCAGAAAAAAAGGATCTCAAGAAGATCCTTTGATCTTTT |
| | | CTACGGGGTCTGACGCTCAGTGGAACGAAAACTCACGTTAAGGGATTTTGGTCATG |
| 15 | ORF3 | atgtttgtttttcttgttttattgccactagtctctagtcagtgtgttaatcttacaaccagaactcaattaccccctgcatacactaattctttcacacgtggtgtttattaccctgacaa |
| | | agttttcagatcctcagttttacattcaactcaggacttgttcttacctttcttttccaatgttacttggttccatgctatacatgtctctgggaccaatggtactaagaggtttgataacc |
| | | ctgtcctaccatttaatgatggtgtttattttgcttccactgagaagtctaacataataagaggctggatttttggtactactttagattcgaagacccagtccctacttattgttaataac |
| | | gctactaatgttgttattaaagtctgtgaatttcaattttgtaatgatccatttttgggtgtttattaccacaaaaacaacaaaagttggatggaaagtgagttcagagtttattctagtgc |
| | | gaataattgcacttttgaatatgtctctcagccttttcttatggaccttgaaggaaaacagggtaatttcaaaaatcttagggaatttgtgtttaagaatattgatggttattttaaaatat |
| | | attctaagcacacgcctattaatttagtgcgtgatctccctcagggtttttcggctttagaaccattggtagatttgccaataggtattaacatcactaggtttcaaactttacttgcttta |
| | | catagaagttatttgactcctggtgattcttcttcaggttggacagctggtgctgcagcttattatgtgggttatcttcaacctaggacttttctattaaaatataatgaaaatggaaccat |
| | | tacagatgctgtagactgtgcacttgaccctctctcagaaacaaagtgtacgttgaaatccttcactgtagaaaaaggaatctatcaaacttctaactttagagtccaaccaacaga |
| | | atctattgttagatttcctaatattacaaacttgtgcccttttggtgaagtttttaacgccaccagatttgcatctgtttatgcttggaacaggaagagaatcagcaactgtgttgctgatt |
| | | attctgtcctatataattccgcatcattttccacttttaagtgttatggagtgtctcctactaaattaaatgatctctgctttactaatgtctatgcagattcatttgtaattagaggtgat |
| | | gaagtcagacaaatcgctccagggcaaactggaaagattgctgattataattataaattaccagatgattttacaggctgcgttatagcttggaattctaacaatcttgattctaaggttg |
| | | gtggtaattataattacctgtatagattgtttaggaagtctaatctcaaaccttttgagagagatatttcaactgaaatctatcaggccggtagcacaccttgtaatggtgttgaaggt |
| | | tttaattgttactttcctttacaatcatatggtttccaacccactaatggtgttggttaccaaccatacagagtagtagtactttcttttgaacttctacatgcaccagcaactgtttgtgg |
| | | acctaaaaagtctactaatttggttaaaaacaaatgtgtcaatttcaacttcaatggtttaacaggcacaggtgttcttactgagtctaacaaaaagtttctgcctttccaacaatttgg |
| | | cagagacattgctgacactactgatgctgtccgtgatccacagacacttgagattcttgacattacaccatgttcttttggtggtgtcagtgttataacaccaggaacaaatacttct |
| | | aaccaggttgctgttctttatcaggatgttaactgcacagaagtccctgttgctattcatgcagatcaacttactcctacttggcgtgtttattctacaggttctaatgtttttcaaacacg |
| | | tgcaggctgtttaataggggctgaacatgtcaacaactcatatgagtgtgacatacccattggtgcaggtatatgcgctagttatcagactcagactaattctcctggcagcgcc |
| | | agcagtgtagctagtcaatccatcattgcctacactatgtcacttggtgcagaaaattcagttgcttactctaataactctattgccatacccacaaattttactattagtgttaccaca |
| | | gaaattctaccagtgtctatgaccaagacatcagtagattgtacaatgtacatttgtggtgattcaactgaatgcagcaatcttttgttgcaatatggcagtttttgtacacaattaaa |
| | | ccgtgctttaactggaatagctgttgaacaagacaaaaacacccaagaagtttttgcacaagtcaaacaaatttacaaaacaccaccaattaaagattttggtggttttaattttt |
| | | cacaaatattaccagatccatcaaaaccaagcaagaggtcatttattgaagatctacttttcaacaaagtgacacttgcagatgctggcttcatcaaacaatatggtgattgcctt |
| | | ggtgatattgctgctagggacctcatttgtgcacaaaagtttaacggccttactgttttgccacctttgctcacagatgaaatgattgctcaatacacttctgcactgttagcgggtac |
| | | aatcacttctggttggacctttggtgcaggtgctgcattacaaataccatttgctatgcaaatggcttataggtttaatggtattggagttacacagaatgttctctatgagaaccaaa |
| | | aattgattgccaaccaatttaatagtgctattggcaaaattcaagactcactttcttccacagcaagtgcacttggaaaacttcaagatgtggtcaaccaaaatgcacaagcttta |
| | | aacacgcttgttaaacaacttagctccaattttggtgcaatttcaagtgttttaaatgatatcctttcacgtcttgaccctcccgaggctgaagtgcaaattgataggttgatcacagg |
| | | cagacttcaaagtttgcagacatatgtgactcaacaattaattagagctgcagaaatcagagcttctgctaatcttgctgctactaaaatgtcagagtgtgtacttggacaatcaa |
| | | aaagagttgatttttgtggaaagggctatcatcttatgtccttccctcagtcagcacctcatggtgtagtcttcttgcatgtgacttatgtccctgcacaagaaaagaacttcacaact |
| | | gctcctgccatttgtcatgatggaaaagcacactttcctcgtgaaggtgtctttgtttcaaatggcacacactggtttgtaacacaaaggaatttttatgaaccacaaatcattacta |
| | | cagacaacacatttgtgtctggtaactgtgatgttgtaataggaattgtcaacaacacagtttatgatcctttgcaacctgaattagactcattcaaggaggagttagataaatattt |
| | | taagaatcatacatcaccagatgttgatttaggtgacatctctggcattaatgcttcagttgtaaacattcaaaaagaaattgaccgcctcaatgaggttgccaagaatttaaatg |
| | | aatctctcatcgatctccaagaacttggaaagtatgagcagtatataaaatggccatggtacatttggctaggttttatagctggcttgattgccatagtaatggtgacaattatgctt |
| | | tgctgtatgaccagttgctgtagttgtctcaagggctgttgttcttgtggatcctgctgcaaatttgatgaagacgactctgagccagtgctcaaaggagtcaaattacattacaca |
| | | TAA |
| 16 | p5 | AGATTATCAAAAAGGATCTTCACCTAGATCCTTTTAAATTAAAAATGAAGTTTTAAATCAATCTAAAGTATATATGAGTAA |
| | | ACTTGGTCTGACAGTTATTAGAAAAATTCATCCAGCAGACGATAAAACGCAATACGCTGGCTATCCGGTGCCGCAATG |
| | | CCATACAGCACCAGAAAACGATCCGCCCATTCGCCGCCCAGTTCTTCCGCAATATCACGGGTGGCCAGCGCAATATC |
| | | CTGATAACGATCCGCCACGCCCAGACGGCCGCAATCAATAAAGCCGCTAAAACGGCCATTTTCCACCATAATGTTCG |
| | | GCAGGCACGCATCACCATGGGTCACCACCAGATCTTCGCCATCCGGCATGCTCGCTTTCAGACGCGCAAACAGCTCT |
| | | GCCGGTGCCAGGCCCTGATGTTCTTCATCCAGATCATCCTGATCCACCAGGCCCGCTTCCATACGGGTACGCGCACG |
| | | TTCAATACGATGTTTCGCCTGATGATCAAACGGACAGGTCGCCGGGTCCAGGGTATGCAGACGACGCATGGCATCCG |
| | | CCATAATGCTCACTTTTTCTGCCGGCGCCAGATGGCTAGACAGCAGATCCTGACCCGGCACTTCGCCCAGCAGCAGC |
| | | CAATCACGGCCCGCTTCGGTCACCACATCCAGCACCGCCGCACACGGAACACCGGTGGTGGCCAGCCAGCTCAGAC |
| | | GCGCCGCTTCATCCTGCAGCTCGTTCAGCGCACCGCTCAGATCGGTTTTCACAAACAGCACCGGACGACCCTGCGC |
| | | GCTCAGACGAAACACCGCCGCATCAGAGCAGCCAATGGTCTGCTGCGCCCAATCATAGCCAAACAGACGTTCCACCC |
| | | ACGCTGCCGGGCTACCCGCATGCAGGCCATCCTGTTCAATCATACTCTTCCTTTTTCAATATTATTGAAGCATTTATCA |
| | | GGGTTATTGTCTCATGAGCGGATACATATTTGAATGTATTTAGAAAAATAAACAAATAGGGGTTCCGCGCACATTTCCC |
| | | CGAAAAGTGCCACCTAAATTGTAAGCGTTAATATTTTGTTAAAATTCGCGTTAAATTTTTGTTAAATCAGCTCATTTTTTA |
| | | ACCAATAGGCCGAAATCGGCAAAATCCCTTATAAATCAAAAGAATAGACCGAGATAGGGTTGAGTGGCCGCTACAGG |
| | | GCGCTCCCATTCGCCATTCAGGCTGCGCAACTGTTGGGAAGGGCGTTTCGGTGCGGGCCTCTTCGCTATTACGCCA |
| | | GCTGGCGAAAGGGGGATGTGCTGCAAGGCGATTAAGTTGGGTAACGCCAGGGTTTTCCCAGTCACGACGTTGTAAAA |
| | | CGACGGCCAGTGAGCGCGACGTAATACGACTCACTATAGGGCGAATTGAAGGAAGGCCGTCAAGGCCGCATGGGAA |
| | | GCCCTCGACCGTCGATTGTCCACTGGTCAACAATAGATGACTTACAACTAATCGGAAGGTGCAGAGACTCGACGGGA |
| | | GCTACCCTAACGTCAAGACGAGGGTAAAGAGAGAGTCCAATTCTCAAAGCCAATAGGCAGTAGCGAAAGCTGCAAGA |
| | | GAATGAAAATCCGTTGACCTTAAACGGTCGTGTGGGTTCAAGTCCCTCCACCCCCACGCCGGAAACGCAATAGCCGA |
| | | AAAACAAAAAACAAAAAAAACAAAAAAAAAACCAAAAAAACAAAACACAacgttactggccgaagccgcttggaataaggccggtgtgcgttt |
| | | gtctatatgttattttccaccatattgccgtcttttggcaatgtgagggcccggaaacctggccctgtcttcttgacgagcattcctaggggtctttcccctctcgccaaaggaatgca |
| | | aggtctgttgaatgtcgtgaaggaagcagttcctctggaagcttcttgaagacaaacaacgtctgtagcgaccctttgcaggcagcggaaccccccacctggcgacaggtgc |
| | | ctctgcggccaaaagccacgtgtataagatacacctgcaaaggcggcacaaccccagtgccacgttgtgagttggatagttgtggaaagagtcaaatggctctcctcaagc |
| | | gtattcaacaaggggctgaaggatgcccagaaggtaccccattgtatgggatctgatctggggcctcggtgcacatgctttacatgtgtttagtcgaggttaaaaaacgtctag |
| | | gccccccgaaccacggggacgtggttttcctttgaaaaacacgatgataataGCCACCatgtttgtttttcttgttttattgccactagtctctagtcagtgtgttaatcttacaacc |
| | | agaactcaattaccccctgcatacactaattctttcacacgtggtgtttattaccctgacaaagttttcagatcctcagttttacattcaactcaggacttgttcttacctttcttttccaa |
| | | tgttacttggttccatgctatacatgtctctgggaccaatggtactaagaggtttgataaccctgtcctaccatttaatgatggtgtttattttgcttccactgagaagtctaacataataa |
| | | gaggctggatttttggtactactttagattcgaagacccagtccctacttattgttaataacgctactaatgttgttattaaagtctgtgaatttcaattttgtaatgatccatttttgggt |
| | | gtttattaccacaaaaacaacaaaagttggatggaaagtgagttcagagtttattctagtgcgaataattgcacttttgaatatgtctctcagccttttcttatggaccttgaaggaaaac |
| | | agggtaatttcaaaaatcttagggaatttgtgtttaagaatattgatggttattttaaaatatattctaagcacacgcctattaatttagtgcgtgatctccctcagggtttttcggcttta |
| | | gaaccattggtagatttgccaataggtattaacatcactaggtttcaaactttacttgctttacatagaagttatttgactcctggtgattcttcttcaggttggacagctggtgctgcagc |
| | | ttattatgtgggttatcttcaacctaggacttttctattaaaatataatgaaaatggaaccattacagatgctgtagactgtgcacttgaccctctctcagaaacaaagtgtacgttga |
| | | aatccttcactgtagaaaaaggaatctatcaaacttctaactttagagtccaaccaacagaatctattgttagatttcctaatattacaaacttgtgcccttttggtgaagtttttaacg |
| | | ccaccagatttgcatctgtttatgcttggaacaggaagagaatcagcaactgtgttgctgattattctgtcctatataattccgcatcattttccacttttaagtgttatggagtgtctcct |
| | | actaaattaaatgatctctgctttactaatgtctatgcagattcatttgtaattagaggtgatgaagtcagacaaatcgctccagggcaaactggaaagattgctgattataattata |
| | | aattaccagatgattttacaggctgcgttatagcttggaattctaacaatcttgattctaaggttggtggtaattataattacctgtatagattgtttaggaagtctaatctcaaacctttt |
| | | gagagagatatttcaactgaaatctatcaggccggtagcacaccttgtaatggtgttgaaggttttaattgttactttcctttacaatcatatggtttccaacccactaatggtgttggtt |
| | | accaaccatacagagtagtagtactttcttttgaacttctacatgcaccagcaactgtttgtggacctaaaaagtctactaatttggttaaaaacaaatgtgtcaatttcaacttcaat |
| | | ggtttaacaggcacaggtgttcttactgagtctaacaaaaagtttctgcctttccaacaatttggcagagacattgctgacactactgatgctgtccgtgatccacagacacttga |
| | | gattcttgacattacaccatgttcttttggtggtgtcagtgttataacaccaggaacaaatacttctaaccaggttgctgttctttatcaggatgttaactgcacagaagtccctgttgct |
| | | attcatgcagatcaacttactcctacttggcgtgtttattctacaggttctaatgtttttcaaacacgtgcaggctgtttaataggggctgaacatgtcaacaactcatatgagtgtgac |
| | | atacccattggtgcaggtatatgcgctagttatcagactcagactaattctcctggcagcgccagcagtgtagctagtcaatccatcattgcctacactatgtcacttggtgcaga |
| | | aaattcagttgcttactctaataactctattgccatacccacaaattttactattagtgttaccacagaaattctaccagtgtctatgaccaagacatcagtagattgtacaatgtaca |
| | | tttgtggtgattcaactgaatgcagcaatcttttgttgcaatatggcagtttttgtacacaattaaaccgtgctttaactggaatagctgttgaacaagacaaaaacacccaagaag |
| | | tttttgcacaagtcaaacaaatttacaaaacaccaccaattaaagattttggtggttttaatttttcacaaatattaccagatccatcaaaaccaagcaagaggtcatttattgaag |
| | | atctacttttcaacaaagtgacacttgcagatgctggcttcatcaaacaatatggtgattgccttggtgatattgctgctagggacctcatttgtgcacaaaagtttaacggccttact |
| | | gttttgccacctttgctcacagatgaaatgattgctcaatacacttctgcactgttagcgggtacaatcacttctggttggacctttggtgcaggtgctgcattacaaataccatttgct |
| | | atgcaaatggcttataggtttaatggtattggagttacacagaatgttctctatgagaaccaaaaattgattgccaaccaatttaatagtgctattggcaaaattcaagactcacttt |
| | | cttccacagcaagtgcacttggaaaacttcaagatgtggtcaaccaaaatgcacaagctttaaacacgcttgttaaacaacttagctccaattttggtgcaatttcaagtgttttaa |
| | | atgatatcctttcacgtcttgaccctcccgaggctgaagtgcaaattgataggttgatcacaggcagacttcaaagtttgcagacatatgtgactcaacaattaattagagctgca |
| | | gaaatcagagcttctgctaatcttgctgctactaaaatgtcagagtgtgtacttggacaatcaaaaagagttgatttttgtggaaagggctatcatcttatgtccttccctcagtcag |
| | | cacctcatggtgtagtcttcttgcatgtgacttatgtccctgcacaagaaaagaacttcacaactgctcctgccatttgtcatgatggaaaagcacactttcctcgtgaaggtgtcttt |
| | | gtttcaaatggcacacactggtttgtaacacaaaggaatttttatgaaccacaaatcattactacagacaacacatttgtgtctggtaactgtgatgttgtaataggaattgtcaac |
| | | aacacagtttatgatcctttgcaacctgaattagactcattcaaggaggagttagataaatattttaagaatcatacatcaccagatgttgatttaggtgacatctctggcattaatg |
| | | cttcagttgtaaacattcaaaaagaaattgaccgcctcaatgaggttgccaagaatttaaatgaatctctcatcgatctccaagaacttggaaagtatgagcagtatataaaatg |
| | | gccatggtacatttggctaggttttatagctggcttgattgccatagtaatggtgacaattatgctttgctgtTAAAAAAAACAAAAAACAAAACGGCTATTATG |
| | | CGTTACCGGCGAGACGCTACGGACTTAAATAATTGAGCCTTAAAGAAGAAATTCTTTAAGTGGATGCTCTCAAACTCA |
| | | GGGAAACCTAAATCTAGTTATAGACAAGGCAATCCTGAGCCAAGCCGAAGTAGTAATTAGTAAGACCAGTGGACAATC |
| | | GACGGATAACAGCATATCTAGCTGGGCCTCATGGGCCTTCCTTTCACTGCCCGCTTTCCAGTCGGGAAACCTGTCGT |
| | | GCCAGCTGCATTAACATGGTCATAGCTGTTTCCTTGCGTATTGGGCGCTCTCCGCTTCCTCGCTCACTGACTCGCTGC |
| | | GCTCGGTCGTTCGGGTAAAGCCTGGGGTGCCTAATGAGCAAAAGGCCAGCAAAAGGCCAGGAACCGTAAAAAGGCC |
| | | GCGTTGCTGGCGTTTTTCCATAGGCTCCGCCCCCCTGACGAGCATCACAAAAATCGACGCTCAAGTCAGAGGTGGCG |
| | | AAACCCGACAGGACTATAAAGATACCAGGCGTTTCCCCCTGGAAGCTCCCTCGTGCGCTCTCCTGTTCCGACCCTGC |
| | | CGCTTACCGGATACCTGTCCGCCTTTCTCCCTTCGGGAAGCGTGGCGCTTTCTCATAGCTCACGCTGTAGGTATCTCA |
| | | GTTCGGTGTAGGTCGTTCGCTCCAAGCTGGGCTGTGTGCACGAACCCCCCGTTCAGCCCGACCGCTGCGCCTTATC |
| | | CGGTAACTATCGTCTTGAGTCCAACCCGGTAAGACACGACTTATCGCCACTGGCAGCAGCCACTGGTAACAGGATTA |
| | | GCAGAGCGAGGTATGTAGGCGGTGCTACAGAGTTCTTGAAGTGGTGGCCTAACTACGGCTACACTAGAAGAACAGTA |
| | | TTTGGTATCTGCGCTCTGCTGAAGCCAGTTACCTTCGGAAAAAGAGTTGGTAGCTCTTGATCCGGCAAACAAACCACC |
| | | GCTGGTAGCGGTGGTTTTTTTGTTTGCAAGCAGCAGATTACGCGCAGAAAAAAAGGATCTCAAGAAGATCCTTTGATC |
| | | TTTTCTACGGGGTCTGACGCTCAGTGGAACGAAAACTCACGTTAAGGGATTTTGGTCATG |
| 17 | ORF5 | atgtttgtttttcttgttttattgccactagtctctagtcagtgtgttaatcttacaaccagaactcaattaccccctgcatacactaattctttcacacgtggtgtttattaccctgacaa |
| | | agttttcagatcctcagttttacattcaactcaggacttgttcttacctttcttttccaatgttacttggttccatgctatacatgtctctgggaccaatggtactaagaggtttgataacc |
| | | ctgtcctaccatttaatgatggtgtttattttgcttccactgagaagtctaacataataagaggctggatttttggtactactttagattcgaagacccagtccctacttattgttaataac |
| | | gctactaatgttgttattaaagtctgtgaatttcaattttgtaatgatccatttttgggtgtttattaccacaaaaacaacaaaagttggatggaaagtgagttcagagtttattctagtgc |
| | | gaataattgcacttttgaatatgtctctcagccttttcttatggaccttgaaggaaaacagggtaatttcaaaaatcttagggaatttgtgtttaagaatattgatggttattttaaaatat |
| | | attctaagcacacgcctattaatttagtgcgtgatctccctcagggtttttcggctttagaaccattggtagatttgccaataggtattaacatcactaggtttcaaactttacttgcttta |
| | | catagaagttatttgactcctggtgattcttcttcaggttggacagctggtgctgcagcttattatgtgggttatcttcaacctaggacttttctattaaaatataatgaaaatggaaccat |
| | | tacagatgctgtagactgtgcacttgaccctctctcagaaacaaagtgtacgttgaaatccttcactgtagaaaaaggaatctatcaaacttctaactttagagtccaaccaacaga |
| | | atctattgttagatttcctaatattacaaacttgtgcccttttggtgaagtttttaacgccaccagatttgcatctgtttatgcttggaacaggaagagaatcagcaactgtgttgctgatt |
| | | attctgtcctatataattccgcatcattttccacttttaagtgttatggagtgtctcctactaaattaaatgatctctgctttactaatgtctatgcagattcatttgtaattagaggtgat |
| | | gaagtcagacaaatcgctccagggcaaactggaaagattgctgattataattataaattaccagatgattttacaggctgcgttatagcttggaattctaacaatcttgattctaaggttg |
| | | gtggtaattataattacctgtatagattgtttaggaagtctaatctcaaaccttttgagagagatatttcaactgaaatctatcaggccggtagcacaccttgtaatggtgttgaaggt |
| | | tttaattgttactttcctttacaatcatatggtttccaacccactaatggtgttggttaccaaccatacagagtagtagtactttcttttgaacttctacatgcaccagcaactgtttgtgg |
| | | acctaaaaagtctactaatttggttaaaaacaaatgtgtcaatttcaacttcaatggtttaacaggcacaggtgttcttactgagtctaacaaaaagtttctgcctttccaacaatttgg |
| | | cagagacattgctgacactactgatgctgtccgtgatccacagacacttgagattcttgacattacaccatgttcttttggtggtgtcagtgttataacaccaggaacaaatacttct |
| | | aaccaggttgctgttctttatcaggatgttaactgcacagaagtccctgttgctattcatgcagatcaacttactcctacttggcgtgtttattctacaggttctaatgtttttcaaacacg |
| | | tgcaggctgtttaataggggctgaacatgtcaacaactcatatgagtgtgacatacccattggtgcaggtatatgcgctagttatcagactcagactaattctcctggcagcgcc |
| | | agcagtgtagctagtcaatccatcattgcctacactatgtcacttggtgcagaaaattcagttgcttactctaataactctattgccatacccacaaattttactattagtgttaccaca |
| | | gaaattctaccagtgtctatgaccaagacatcagtagattgtacaatgtacatttgtggtgattcaactgaatgcagcaatcttttgttgcaatatggcagtttttgtacacaattaaa |
| | | ccgtgctttaactggaatagctgttgaacaagacaaaaacacccaagaagtttttgcacaagtcaaacaaatttacaaaacaccaccaattaaagattttggtggttttaattttt |
| | | cacaaatattaccagatccatcaaaaccaagcaagaggtcatttattgaagatctacttttcaacaaagtgacacttgcagatgctggcttcatcaaacaatatggtgattgcctt |
| | | ggtgatattgctgctagggacctcatttgtgcacaaaagtttaacggccttactgttttgccacctttgctcacagatgaaatgattgctcaatacacttctgcactgttagcgggtac |
| | | aatcacttctggttggacctttggtgcaggtgctgcattacaaataccatttgctatgcaaatggcttataggtttaatggtattggagttacacagaatgttctctatgagaaccaaa |
| | | aattgattgccaaccaatttaatagtgctattggcaaaattcaagactcactttcttccacagcaagtgcacttggaaaacttcaagatgtggtcaaccaaaatgcacaagcttta |
| | | aacacgcttgttaaacaacttagctccaattttggtgcaatttcaagtgttttaaatgatatcctttcacgtcttgaccctcccgaggctgaagtgcaaattgataggttgatcacagg |
| | | cagacttcaaagtttgcagacatatgtgactcaacaattaattagagctgcagaaatcagagcttctgctaatcttgctgctactaaaatgtcagagtgtgtacttggacaatcaa |
| | | aaagagttgatttttgtggaaagggctatcatcttatgtccttccctcagtcagcacctcatggtgtagtcttcttgcatgtgacttatgtccctgcacaagaaaagaacttcacaact |
| | | gctcctgccatttgtcatgatggaaaagcacactttcctcgtgaaggtgtctttgtttcaaatggcacacactggtttgtaacacaaaggaatttttatgaaccacaaatcattacta |
| | | cagacaacacatttgtgtctggtaactgtgatgttgtaataggaattgtcaacaacacagtttatgatcctttgcaacctgaattagactcattcaaggaggagttagataaatattt |
| | | taagaatcatacatcaccagatgttgatttaggtgacatctctggcattaatgcttcagttgtaaacattcaaaaagaaattgaccgcctcaatgaggttgccaagaatttaaatg |
| | | aatctctcatcgatctccaagaacttggaaagtatgagcagtatataaaatggccatggtacatttggctaggttttatagctggcttgattgccatagtaatggtgacaattatgctt |
| | | tgctgtTAA |
| 18 | P7 | AGATTATCAAAAAGGATCTTCACCTAGATCCTTTTAAATTAAAAATGAAGTTTTAAATCAATCTAAAGTATATATGAGTAA |
| | | ACTTGGTCTGACAGTTATTAGAAAAATTCATCCAGCAGACGATAAAACGCAATACGCTGGCTATCCGGTGCCGCAATG |
| | | CCATACAGCACCAGAAAACGATCCGCCCATTCGCCGCCCAGTTCTTCCGCAATATCACGGGTGGCCAGCGCAATATC |
| | | CTGATAACGATCCGCCACGCCCAGACGGCCGCAATCAATAAAGCCGCTAAAACGGCCATTTTCCACCATAATGTTCG |
| | | GCAGGCACGCATCACCATGGGTCACCACCAGATCTTCGCCATCCGGCATGCTCGCTTTCAGACGCGCAAACAGCTCT |
| | | GCCGGTGCCAGGCCCTGATGTTCTTCATCCAGATCATCCTGATCCACCAGGCCCGCTTCCATACGGGTACGCGCACG |
| | | TTCAATACGATGTTTCGCCTGATGATCAAACGGACAGGTCGCCGGGTCCAGGGTATGCAGACGACGCATGGCATCCG |
| | | CCATAATGCTCACTTTTTCTGCCGGCGCCAGATGGCTAGACAGCAGATCCTGACCCGGCACTTCGCCCAGCAGCAGC |
| | | CAATCACGGCCCGCTTCGGTCACCACATCCAGCACCGCCGCACACGGAACACCGGTGGTGGCCAGCCAGCTCAGAC |
| | | GCGCCGCTTCATCCTGCAGCTCGTTCAGCGCACCGCTCAGATCGGTTTTCACAAACAGCACCGGACGACCCTGCGC |
| | | GCTCAGACGAAACACCGCCGCATCAGAGCAGCCAATGGTCTGCTGCGCCCAATCATAGCCAAACAGACGTTCCACCC |
| | | ACGCTGCCGGGCTACCCGCATGCAGGCCATCCTGTTCAATCATACTCTTCCTTTTTCAATATTATTGAAGCATTTATCA |
| | | GGGTTATTGTCTCATGAGCGGATACATATTTGAATGTATTTAGAAAAATAAACAAATAGGGGTTCCGCGCACATTTCCC |
| | | CGAAAAGTGCCACCTAAATTGTAAGCGTTAATATTTTGTTAAAATTCGCGTTAAATTTTTGTTAAATCAGCTCATTTTTTA |
| | | ACCAATAGGCCGAAATCGGCAAAATCCCTTATAAATCAAAAGAATAGACCGAGATAGGGTTGAGTGGCCGCTACAGG |
| | | GCGCTCCCATTCGCCATTCAGGCTGCGCAACTGTTGGGAAGGGCGTTTCGGTGCGGGCCTCTTCGCTATTACGCCA |
| | | GCTGGCGAAAGGGGGATGTGCTGCAAGGCGATTAAGTTGGGTAACGCCAGGGTTTTCCCAGTCACGACGTTGTAAAA |
| | | CGACGGCCAGTGAGCGCGACGTAATACGACTCACTATAGGGCGAATTGAAGGAAGGCCGTCAAGGCCGCATGGGAA |
| | | GCCCTCGACCGTCGATTGTCCACTGGTCAACAATAGATGACTTACAACTAATCGGAAGGTGCAGAGACTCGACGGGA |
| | | GCTACCCTAACGTCAAGACGAGGGTAAAGAGAGAGTCCAATTCTCAAAGCCAATAGGCAGTAGCGAAAGCTGCAAGA |
| | | GAATGAAAATCCGTTGACCTTAAACGGTCGTGTGGGTTCAAGTCCCTCCACCCCCACGCCGGAAACGCAATAGCCGA |
| | | AAAACAAAAAACAAAAAAAACAAAAAAAAAACCAAAAAAACAAAACACAacgttactggccgaagccgcttggaataaggccggtgtgcgttt |
| | | gtctatatgttattttccaccatattgccgtcttttggcaatgtgagggcccggaaacctggccctgtcttcttgacgagcattcctaggggtctttcccctctcgccaaaggaatgca |
| | | aggtctgttgaatgtcgtgaaggaagcagttcctctggaagcttcttgaagacaaacaacgtctgtagcgaccctttgcaggcagcggaaccccccacctggcgacaggtgc |
| | | ctctgcggccaaaagccacgtgtataagatacacctgcaaaggcggcacaaccccagtgccacgttgtgagttggatagttgtggaaagagtcaaatggctctcctcaagc |
| | | gtattcaacaaggggctgaaggatgcccagaaggtaccccattgtatgggatctgatctggggcctcggtgcacatgctttacatgtgtttagtcgaggttaaaaaacgtctag |
| | | gccccccgaaccacggggacgtggttttcctttgaaaaacacgatgataataGCCACCatgtttgtttttcttgttttattgccactagtctctagtcagtgtgttaatcttacaacc |
| | | agaactcaattaccccctgcatacactaattctttcacacgtggtgtttattaccctgacaaagttttcagatcctcagttttacattcaactcaggatttgttcttacctttttttccaat |
| | | gttacttggttccatgctatacatgtctctgggaccaatggtactaagaggtttgataaccctgtcctaccatttaatgatggtgtttattttgcttccactgagaagtctaacataataag |
| | | aggctggatctttggtactactttagattcgaagacccagtccctacttattgttaataacgctactaatgttgttattaaagtctgtgaatttcaattttgtaatgatccatttttgggtg |
| | | tttattaccacaaaaacaacaaaagttggatggaaagtgagttcagagtttattctagtgcgaataattgcacttttgaatatgtctctcagccttttcttatggaccttgaaggaaaac |
| | | agggtaatttcaaaaatcttagggaatttgtgtttaagaatattgatggttattttaaaatatattctaagcacacgcctattaatttagtgcgtgatctccctcagggtttttcggcttta |
| | | gaaccattggtagatttgccaataggtattaacatcactaggtttcaaactttacttgctttacatagaagttatttgactcctggtgattcttcttcaggttggacagctggtgctgcagc |
| | | ttattatgtgggttatcttcaacctaggacttttctattaaaatataatgaaaatggaaccattacagatgctgtagactgtgcacttgaccctctctcagaaacaaagtgtacgttga |
| | | aatccttcactgtagaaaaaggaatctatcaaacttctaactttagagtccaaccaacagaatctattgttagatttcctaatattacaaacttgtgcccttttggtgaagtttttaacg |
| | | ccaccagatttgcatctgtttatgcttggaacaggaagagaatcagcaactgtgttgctgattattctgtcctatataattccgcatcattttccacttttaagtgttatggagtgtctcct |
| | | actaaattaaatgatctctgctttactaatgtctatgcagattcatttgtaattagaggtgatgaagtcagacaaatcgctccagggcaaactggaaagattgctgattataattata |
| | | aattaccagatgattttacaggctgcgttatagcttggaattctaacaatcttgattctaaggttggtggtaattataattacctgtatagattgtttaggaagtctaatctcaaacctttt |
| | | gagagagatatttcaactgaaatctatcaggccggtagcacaccttgtaatggtgttgaaggttttaattgttactttcctttacaatcatatggtttccaacccactaatggtgttggtt |
| | | accaaccatacagagtagtagtactttcttttgaacttctacatgcaccagcaactgtttgtggacctaaaaagtctactaatttggttaaaaacaaatgtgtcaatttcaacttcaat |
| | | ggtttaacaggcacaggtgttcttactgagtctaacaaaaagtttctgcctttccaacaatttggcagagacattgctgacactactgatgctgtccgtgatccacagacacttga |
| | | gattcttgacattacaccatgttcttttggtggtgtcagtgttataacaccaggaacaaatacttctaaccaggttgctgttctttatcaggatgttaactgcacagaagtccctgttgct |
| | | attcatgcagatcaacttactcctacttggcgtgtttattctacaggttctaatgtttttcaaacacgtgcaggctgtttaataggggctgaacatgtcaacaactcatatgagtgtgac |
| | | atacccattggtgcaggtatatgcgctagttatcagactcagactaattctcctggcagcgccagcagtgtagctagtcaatccatcattgcctacactatgtcacttggtgcaga |
| | | aaattcagttgcttactctaataactctattgccatacccacaaattttactattagtgttaccacagaaattctaccagtgtctatgaccaagacatcagtagattgtacaatgtaca |
| | | tttgtggtgattcaactgaatgcagcaatcttttgttgcaatatggcagtttttgtacacaattaaaccgtgctttaactgggatagctgttgaacaagacaaaaacacccaagaag |
| | | tttttgcacaagtcaaacaaatttacaaaacaccaccaattaaagattttggtggttttaatttttcacaaatattaccagatccatcaaaaccaagcaagaggtcatttattgaag |
| | | atctacttttcaacaaagtgacacttgcagatgctggcttcatcaaacaatatggtgattgccttggtgatattgctgctagggacctcatttgtgcacaaaagtttaacggccttact |
| | | gttttgccacctttgctcacagatgaaatgattgctcaatacacttctgcactgttagcgggtacaatcacttctggttggacctttggtgcaggtgctgcattacaaataccatttgct |
| | | atgcaaatggcttataggtttaatggtattggagttacacagaatgttctctatgagaaccaaaaattgattgccaaccaatttaatagtgccattggcaaaattcaagactcacttt |
| | | cttccacagcaagtgcacttggaaaacttcaagatgtggtcaaccaaaatgcacaagctttaaacacgcttgttaaacaacttagctccaattttggtgcaatttcaagtgttttaa |
| | | atgatatcctttcacgtcttgaccctcccgaggctgaagtgcaaattgataggttgatcacaggcagacttcaaagtttgcagacatatgtgactcaacaattaattagagctgca |
| | | gaaatcagagcttctgctaatcttgctgctactaaaatgtcagagtgtgtacttggacaatcaaaaagagttgatttttgtggaaagggctatcatcttatgtccttccctcagtcag |
| | | cacctcatggtgtagtcttcttgcatgtgacttatgtccctgcacaagaaaagaacttcacaactgctcctgccatttgtcatgatggaaaagcacactttcctcgtgaaggtgtcttt |
| | | gtttcaaatggcacacactggtttgtaacacaaaggaatttttatgaaccacaaatcattactacagacaacacatttgtgtctggtaactgtgatgttgtaataggaattgtcaac |
| | | aacacagtttatgatcctttgcaacctgaattagactcattcaaggaggagttagataaatattttaagaatcatacatcaccagatgttgatttaggtgacatctctggcattaatg |
| | | cttcagttgtaaacattcaaaaagaaattgaccgcctcaatgaggttgccaagaatttaaatgaatctctcatcgatctccaagaacttggaaagtatgagcagGGGTATA |
| | | TCCCTGAAGCCCCCAGGGACGGCCAGGCTTACGTCAGAAAGGATGGAGAGTGGGTGCTCTTGAGCACCTTCCTGTA |
| | | AAAAAAACAAAAAACAAAACGGCTATTATGCGTTACCGGCGAGACGCTACGGACTTAAATAATTGAGCCTTAAAGAAG |
| | | AAATTCTTTAAGTGGATGCTCTCAAACTCAGGGAAACCTAAATCTAGTTATAGACAAGGCAATCCTGAGCCAAGCCGA |
| | | AGTAGTAATTAGTAAGACCAGTGGACAATCGACGGATAACAGCATATCTAGCTGGGCCTCATGGGCCTTCCTTTCACT |
| | | GCCCGCTTTCCAGTCGGGAAACCTGTCGTGCCAGCTGCATTAACATGGTCATAGCTGTTTCCTTGCGTATTGGGCGC |
| | | TCTCCGCTTCCTCGCTCACTGACTCGCTGCGCTCGGTCGTTCGGGTAAAGCCTGGGGTGCCTAATGAGCAAAAGGCC |
| | | AGCAAAAGGCCAGGAACCGTAAAAAGGCCGCGTTGCTGGCGTTTTTCCATAGGCTCCGCCCCCCTGACGAGCATCAC |
| | | AAAAATCGACGCTCAAGTCAGAGGTGGCGAAACCCGACAGGACTATAAAGATACCAGGCGTTTCCCCCTGGAAGCTC |
| | | CCTCGTGCGCTCTCCTGTTCCGACCCTGCCGCTTACCGGATACCTGTCCGCCTTTCTCCCTTCGGGAAGCGTGGCGC |
| | | TTTCTCATAGCTCACGCTGTAGGTATCTCAGTTCGGTGTAGGTCGTTCGCTCCAAGCTGGGCTGTGTGCACGAACCC |
| | | CCCGTTCAGCCCGACCGCTGCGCCTTATCCGGTAACTATCGTCTTGAGTCCAACCCGGTAAGACACGACTTATCGCC |
| | | ACTGGCAGCAGCCACTGGTAACAGGATTAGCAGAGCGAGGTATGTAGGCGGTGCTACAGAGTTCTTGAAGTGGTGG |
| | | CCTAACTACGGCTACACTAGAAGAACAGTATTTGGTATCTGCGCTCTGCTGAAGCCAGTTACCTTCGGAAAAAGAGTT |
| | | GGTAGCTCTTGATCCGGCAAACAAACCACCGCTGGTAGCGGTGGTTTTTTTGTTTGCAAGCAGCAGATTACGCGCAG |
| | | AAAAAAAGGATCTCAAGAAGATCCTTTGATCTTTTCTACGGGGTCTGACGCTCAGTGGAACGAAAACTCACGTTAAGG |
| | | GATTTTGGTCATG |
| 19 | ORF7 | atgtttgtttttcttgttttattgccactagtctctagtcagtgtgttaatcttacaaccagaactcaattaccccctgcatacactaattctttcacacgtggtgtttattaccctgacaa |
| | | agttttcagatcctcagttttacattcaactcaggatttgttcttacctttcttttccaatgttacttggttccatgctatacatgtctctgggaccaatggtactaagaggtttgataacc |
| | | ctgtcctaccatttaatgatggtgtttattttgcttccactgagaagtctaacataataagaggctggatctttggtactactttagattcgaagacccagtccctacttattgttaataac |
| | | gctactaatgttgttattaaagtctgtgaatttcaattttgtaatgatccatttttgggtgtttattaccacaaaaacaacaaaagttggatggaaagtgagttcagagtttattctagtgc |
| | | gaataattgcacttttgaatatgtctctcagccttttcttatggaccttgaaggaaaacagggtaatttcaaaaatcttagggaatttgtgtttaagaatattgatggttattttaaaatat |
| | | attctaagcacacgcctattaatttagtgcgtgatctccctcagggtttttcggctttagaaccattggtagatttgccaataggtattaacatcactaggtttcaaactttacttgcttta |
| | | catagaagttatttgactcctggtgattcttcttcaggttggacagctggtgctgcagcttattatgtgggttatcttcaacctaggacttttctattaaaatataatgaaaatggaaccat |
| | | tacagatgctgtagactgtgcacttgaccctctctcagaaacaaagtgtacgttgaaatccttcactgtagaaaaaggaatctatcaaacttctaactttagagtccaaccaacaga |
| | | atctattgttagatttcctaatattacaaacttgtgcccttttggtgaagtttttaacgccaccagatttgcatctgtttatgcttggaacaggaagagaatcagcaactgtgttgctgatt |
| | | attctgtcctatataattccgcatcattttccacttttaagtgttatggagtgtctcctactaaattaaatgatctctgctttactaatgtctatgcagattcatttgtaattagaggtgat |
| | | gaagtcagacaaatcgctccagggcaaactggaaagattgctgattataattataaattaccagatgattttacaggctgcgttatagcttggaattctaacaatcttgattctaaggttg |
| | | gtggtaattataattacctgtatagattgtttaggaagtctaatctcaaaccttttgagagagatatttcaactgaaatctatcaggccggtagcacaccttgtaatggtgttgaaggt |
| | | tttaattgttactttcctttacaatcatatggtttccaacccactaatggtgttggttaccaaccatacagagtagtagtactttcttttgaacttctacatgcaccagcaactgtttgtgg |
| | | acctaaaaagtctactaatttggttaaaaacaaatgtgtcaatttcaacttcaatggtttaacaggcacaggtgttcttactgagtctaacaaaaagtttctgcctttccaacaatttgg |
| | | cagagacattgctgacactactgatgctgtccgtgatccacagacacttgagattcttgacattacaccatgttcttttggtggtgtcagtgttataacaccaggaacaaatacttct |
| | | aaccaggttgctgttctttatcaggatgttaactgcacagaagtccctgttgctattcatgcagatcaacttactcctacttggcgtgtttattctacaggttctaatgtttttcaaacacg |
| | | tgcaggctgtttaataggggctgaacatgtcaacaactcatatgagtgtgacatacccattggtgcaggtatatgcgctagttatcagactcagactaattctcctggcagcgcc |
| | | agcagtgtagctagtcaatccatcattgcctacactatgtcacttggtgcagaaaattcagttgcttactctaataactctattgccatacccacaaattttactattagtgttaccaca |
| | | gaaattctaccagtgtctatgaccaagacatcagtagattgtacaatgtacatttgtggtgattcaactgaatgcagcaatcttttgttgcaatatggcagtttttgtacacaattaaa |
| | | ccgtgctttaactgggatagctgttgaacaagacaaaaacacccaagaagtttttgcacaagtcaaacaaatttacaaaacaccaccaattaaagattttggtggttttaattttt |
| | | cacaaatattaccagatccatcaaaaccaagcaagaggtcatttattgaagatctacttttcaacaaagtgacacttgcagatgctggcttcatcaaacaatatggtgattgcctt |
| | | ggtgatattgctgctagggacctcatttgtgcacaaaagtttaacggccttactgttttgccacctttgctcacagatgaaatgattgctcaatacacttctgcactgttagcgggtac |
| | | aatcacttctggttggacctttggtgcaggtgctgcattacaaataccatttgctatgcaaatggcttataggtttaatggtattggagttacacagaatgttctctatgagaaccaaa |
| | | aattgattgccaaccaatttaatagtgccattggcaaaattcaagactcactttcttccacagcaagtgcacttggaaaacttcaagatgtggtcaaccaaaatgcacaagcttt |
| | | aaacacgcttgttaaacaacttagctccaattttggtgcaatttcaagtgttttaaatgatatcctttcacgtcttgaccctcccgaggctgaagtgcaaattgataggttgatcacag |
| | | gcagacttcaaagtttgcagacatatgtgactcaacaattaattagagctgcagaaatcagagcttctgctaatcttgctgctactaaaatgtcagagtgtgtacttggacaatca |
| | | aaaagagttgatttttgtggaaagggctatcatcttatgtccttccctcagtcagcacctcatggtgtagtcttcttgcatgtgacttatgtccctgcacaagaaaagaacttcacaa |
| | | ctgctcctgccatttgtcatgatggaaaagcacactttcctcgtgaaggtgtctttgtttcaaatggcacacactggtttgtaacacaaaggaatttttatgaaccacaaatcattact |
| | | acagacaacacatttgtgtctggtaactgtgatgttgtaataggaattgtcaacaacacagtttatgatcctttgcaacctgaattagactcattcaaggaggagttagataaatat |
| | | tttaagaatcatacatcaccagatgttgatttaggtgacatctctggcattaatgcttcagttgtaaacattcaaaaagaaattgaccgcctcaatgaggttgccaagaatttaaat |
| | | gaatctctcatcgatctccaagaacttggaaagtatgagcagGGGTATATCCCTGAAGCCCCCAGGGACGGCCAGGCTTACGTCAGAAAG |
| | | GATGGAGAGTGGGTGCTCTTGAGCACCTTCCTGTAA |
| 20 | P9 | AGATTATCAAAAAGGATCTTCACCTAGATCCTTTTAAATTAAAAATGAAGTTTTAAATCAATCTAAAGTATATATGAGTAA |
| | | ACTTGGTCTGACAGTTATTAGAAAAATTCATCCAGCAGACGATAAAACGCAATACGCTGGCTATCCGGTGCCGCAATG |
| | | CCATACAGCACCAGAAAACGATCCGCCCATTCGCCGCCCAGTTCTTCCGCAATATCACGGGTGGCCAGCGCAATATC |
| | | CTGATAACGATCCGCCACGCCCAGACGGCCGCAATCAATAAAGCCGCTAAAACGGCCATTTTCCACCATAATGTTCG |
| | | GCAGGCACGCATCACCATGGGTCACCACCAGATCTTCGCCATCCGGCATGCTCGCTTTCAGACGCGCAAACAGCTCT |
| | | GCCGGTGCCAGGCCCTGATGTTCTTCATCCAGATCATCCTGATCCACCAGGCCCGCTTCCATACGGGTACGCGCACG |
| | | TTCAATACGATGTTTCGCCTGATGATCAAACGGACAGGTCGCCGGGTCCAGGGTATGCAGACGACGCATGGCATCCG |
| | | CCATAATGCTCACTTTTTCTGCCGGCGCCAGATGGCTAGACAGCAGATCCTGACCCGGCACTTCGCCCAGCAGCAGC |
| | | CAATCACGGCCCGCTTCGGTCACCACATCCAGCACCGCCGCACACGGAACACCGGTGGTGGCCAGCCAGCTCAGAC |
| | | GCGCCGCTTCATCCTGCAGCTCGTTCAGCGCACCGCTCAGATCGGTTTTCACAAACAGCACCGGACGACCCTGCGC |
| | | GCTCAGACGAAACACCGCCGCATCAGAGCAGCCAATGGTCTGCTGCGCCCAATCATAGCCAAACAGACGTTCCACCC |
| | | ACGCTGCCGGGCTACCCGCATGCAGGCCATCCTGTTCAATCATACTCTTCCTTTTTCAATATTATTGAAGCATTTATCA |
| | | GGGTTATTGTCTCATGAGCGGATACATATTTGAATGTATTTAGAAAAATAAACAAATAGGGGTTCCGCGCACATTTCCC |
| | | CGAAAAGTGCCACCTAAATTGTAAGCGTTAATATTTTGTTAAAATTCGCGTTAAATTTTTGTTAAATCAGCTCATTTTTTA |
| | | ACCAATAGGCCGAAATCGGCAAAATCCCTTATAAATCAAAAGAATAGACCGAGATAGGGTTGAGTGGCCGCTACAGG |
| | | GCGCTCCCATTCGCCATTCAGGCTGCGCAACTGTTGGGAAGGGCGTTTCGGTGCGGGCCTCTTCGCTATTACGCCA |
| | | GCTGGCGAAAGGGGGATGTGCTGCAAGGCGATTAAGTTGGGTAACGCCAGGGTTTTCCCAGTCACGACGTTGTAAAA |
| | | CGACGGCCAGTGAGCGCGACGTAATACGACTCACTATAGGGCGAATTGAAGGAAGGCCGTCAAGGCCGCATGGGAA |
| | | GCCCTCGACCGTCGATTGTCCACTGGTCAACAATAGATGACTTACAACTAATCGGAAGGTGCAGAGACTCGACGGGA |
| | | GCTACCCTAACGTCAAGACGAGGGTAAAGAGAGAGTCCAATTCTCAAAGCCAATAGGCAGTAGCGAAAGCTGCAAGA |
| | | GAATGAAAATCCGTTGACCTTAAACGGTCGTGTGGGTTCAAGTCCCTCCACCCCCACGCCGGAAACGCAATAGCCGA |
| | | AAAACAAAAAACAAAAAAAACAAAAAAAAAACCAAAAAAACAAAACACAacgttactggccgaagccgcttggaataaggccggtgtgcgttt |
| | | gtctatatgttattttccaccatattgccgtcttttggcaatgtgagggcccggaaacctggccctgtcttcttgacgagcattcctaggggtctttcccctctcgccaaaggaatgca |
| | | aggtctgttgaatgtcgtgaaggaagcagttcctctggaagcttcttgaagacaaacaacgtctgtagcgaccctttgcaggcagcggaaccccccacctggcgacaggtgc |
| | | ctctgcggccaaaagccacgtgtataagatacacctgcaaaggcggcacaaccccagtgccacgttgtgagttggatagttgtggaaagagtcaaatggctctcctcaagc |
| | | gtattcaacaaggggctgaaggatgcccagaaggtaccccattgtatgggatctgatctggggcctcggtgcacatgctttacatgtgtttagtcgaggttaaaaaacgtctag |
| | | gccccccgaaccacggggacgtggttttcctttgaaaaacacgatgataataGCCACCatgtttgtttttcttgttttattgccactagtctctagtcagtgtgttaatcttacaacc |
| | | agaactcaattaccccctgcatacactaattctttcacacgtggtgtttattaccctgacaaagttttcagatcctcagttttacattcaactcaggatttgttcttacctttcttttccaa |
| | | tgttacttggttccatgctatacatgtctctgggaccaatggtactaagaggtttgataaccctgtcctaccatttaatgatggtgtttattttgcttccactgagaagtctaacataataa |
| | | gaggctggatctttggtactactttagattcgaagacccagtccctacttattgttaataacgctactaatgttgttattaaagtctgtgaatttcaattttgtaatgatccatttttgggt |
| | | gtttattaccacaaaaacaacaaaagttggatggaaagtgagttcagagtttattctagtgcgaataattgcacttttgaatatgtctctcagccttttcttatggaccttgaaggaaaac |
| | | agggtaatttcaaaaatcttagggaatttgtgtttaagaatattgatggttattttaaaatatattctaagcacacgcctattaatttagtgcgtgatctccctcagggtttttcggcttta |
| | | gaaccattggtagatttgccaataggtattaacatcactaggtttcaaactttacttgctttacatagaagttatttgactcctggtgattcttcttcaggttggacagctggtgctgcagc |
| | | ttattatgtgggttatcttcaacctaggacttttctattaaaatataatgaaaatggaaccattacagatgctgtagactgtgcacttgaccctctctcagaaacaaagtgtacgttga |
| | | aatccttcactgtagaaaaaggaatctatcaaacttctaactttagagtccaaccaacagaatctattgttagatttcctaatattacaaacttgtgcccttttggtgaagtttttaacg |
| | | ccaccagatttgcatctgtttatgcttggaacaggaagagaatcagcaactgtgttgctgattattctgtcctatataattccgcatcattttccacttttaagtgttatggagtgtctcct |
| | | actaaattaaatgatctctgctttactaatgtctatgcagattcatttgtaattagaggtgatgaagtcagacaaatcgctccagggcaaactggaaagattgctgattataattata |
| | | aattaccagatgattttacaggctgcgttatagcttggaattctaacaatcttgattctaaggttggtggtaattataattacctgtatagattgtttaggaagtctaatctcaaacctttt |
| | | gagagagatatttcaactgaaatctatcaggccggtagcacaccttgtaatggtgttgaaggttttaattgttactttcctttacaatcatatggtttccaacccactaatggtgttggtt |
| | | accaaccatacagagtagtagtactttcttttgaacttctacatgcaccagcaactgtttgtggacctaaaaagtctactaatttggttaaaaacaaatgtgtcaatttcaacttcaat |
| | | ggtttaacaggcacaggtgttcttactgagtctaacaaaaagtttctgcctttccaacaatttggcagagacattgctgacactactgatgctgtccgtgatccacagacacttga |
| | | gattcttgacattacaccatgttcttttggtggtgtcagtgttataacaccaggaacaaatacttctaaccaggttgctgttctttatcaggatgttaactgcacagaagtccctgttgct |
| | | attcatgcagatcaacttactcctacttggcgtgtttattctacaggttctaatgtttttcaaacacgtgcaggctgtttaataggggctgaacatgtcaacaactcatatgagtgtgac |
| | | atacccattggtgcaggtatatgcgctagttatcagactcagactaattctcctggcagcgccagcagtgtagctagtcaatccatcattgcctacactatgtcacttggtgcaga |
| | | aaattcagttgcttactctaataactctattgccatacccacaaattttactattagtgttaccacagaaattctaccagtgtctatgaccaagacatcagtagattgtacaatgtaca |
| | | tttgtggtgattcaactgaatgcagcaatcttttgttgcaatatggcagtttttgtacacaattaaaccgtgctttaactgggatagctgttgaacaagacaaaaacacccaagaag |
| | | tttttgcacaagtcaaacaaatttacaaaacaccaccaattaaagattttggtggttttaatttttcacaaatattaccagatccatcaaaaccaagcaagaggtcatttattgaag |
| | | atctacttttcaacaaagtgacacttgcagatgctggcttcatcaaacaatatggtgattgccttggtgatattgctgctagggacctcatttgtgcacaaaagtttaacggccttact |
| | | gttttgccacctttgctcacagatgaaatgattgctcaatacacttctgcactgttagcgggtacaatcacttctggttggacctttggtgcaggtgctgcattacaaataccatttgct |
| | | atgcaaatggcttataggtttaatggtattggagttacacagaatgttctctatgagaaccaaaaattgattgccaaccaatttaatagtgccattggcaaaattcaagactcacttt |
| | | cttccacagcaagtgcacttggaaaacttcaagatgtggtcaaccaaaatgcacaagctttaaacacgcttgttaaacaacttagctccaattttggtgcaatttcaagtgttttaa |
| | | atgatatcctttcacgtcttgaccctcccgaggctgaagtgcaaattgataggttgatcacaggcagacttcaaagtttgcagacatatgtgactcaacaattaattagagctgca |
| | | gaaatcagagcttctgctaatcttgctgctactaaaatgtcagagtgtgtacttggacaatcaaaaagagttgatttttgtggaaagggctatcatcttatgtccttccctcagtcag |
| | | cacctcatggtgtagtcttcttgcatgtgacttatgtccctgcacaagaaaagaacttcacaactgctcctgccatttgtcatgatggaaaagcacactttcctcgtgaaggtgtcttt |
| | | gtttcaaatggcacacactggtttgtaacacaaaggaatttttatgaaccacaaatcattactacagacaacacatttgtgtctggtaactgtgatgttgtaataggaattgtcaac |
| | | aacacagtttatgatcctttgcaacctgaattagactcattcaaggaggagttagataaatattttaagaatcatacatcaccagatgttgatttaggtgacatctctggcattaatg |
| | | cttcagttgtaaacattcaaaaagaaattgaccgcctcaatgaggttgccaagaatttaaatgaatctctcatcgatctccaagaacttggaaagtatgagcagtatataaaatg |
| | | gccatggtacatttggctaggttttatagctggcttgattgccatagtaatggtgacaattatgctttgctgtatgaccagttgctgtagttgtctcaagggctgttgttcttgtggatcctg |
| | | ctgcaaatttgatgaagacgactctgagccagtgctcaaaggagtcaaattacattacacaTAAAAAAAACAAAAAACAAAACGGCTATTATGCGTT |
| | | ACCGGCGAGACGCTACGGACTTAAATAATTGAGCCTTAAAGAAGAAATTCTTTAAGTGGATGCTCTCAAACTCAGGGA |
| | | AACCTAAATCTAGTTATAGACAAGGCAATCCTGAGCCAAGCCGAAGTAGTAATTAGTAAGACCAGTGGACAATCGACG |
| | | GATAACAGCATATCTAGCTGGGCCTCATGGGCCTTCCTTTCACTGCCCGCTTTCCAGTCGGGAAACCTGTCGTGCCA |
| | | GCTGCATTAACATGGTCATAGCTGTTTCCTTGCGTATTGGGCGCTCTCCGCTTCCTCGCTCACTGACTCGCTGCGCTC |
| | | GGTCGTTCGGGTAAAGCCTGGGGTGCCTAATGAGCAAAAGGCCAGCAAAAGGCCAGGAACCGTAAAAAGGCCGCGT |
| | | TGCTGGCGTTTTTCCATAGGCTCCGCCCCCCTGACGAGCATCACAAAAATCGACGCTCAAGTCAGAGGTGGCGAAAC |
| | | CCGACAGGACTATAAAGATACCAGGCGTTTCCCCCTGGAAGCTCCCTCGTGCGCTCTCCTGTTCCGACCCTGCCGCT |
| | | TACCGGATACCTGTCCGCCTTTCTCCCTTCGGGAAGCGTGGCGCTTTCTCATAGCTCACGCTGTAGGTATCTCAGTTC |
| | | GGTGTAGGTCGTTCGCTCCAAGCTGGGCTGTGTGCACGAACCCCCCGTTCAGCCCGACCGCTGCGCCTTATCCGGT |
| | | AACTATCGTCTTGAGTCCAACCCGGTAAGACACGACTTATCGCCACTGGCAGCAGCCACTGGTAACAGGATTAGCAG |
| | | AGCGAGGTATGTAGGCGGTGCTACAGAGTTCTTGAAGTGGTGGCCTAACTACGGCTACACTAGAAGAACAGTATTTG |
| | | GTATCTGCGCTCTGCTGAAGCCAGTTACCTTCGGAAAAAGAGTTGGTAGCTCTTGATCCGGCAAACAAACCACCGCT |
| | | GGTAGCGGTGGTTTTTTTGTTTGCAAGCAGCAGATTACGCGCAGAAAAAAAGGATCTCAAGAAGATCCTTTGATCTTTT |
| | | CTACGGGGTCTGACGCTCAGTGGAACGAAAACTCACGTTAAGGGATTTTGGTCATG |
| 21 | ORF9 | atgtttgtttttcttgttttattgccactagtctctagtcagtgtgttaatcttacaaccagaactcaattaccccctgcatacactaattctttcacacgtggtgtttattaccctgacaa |
| | | agttttcagatcctcagttttacattcaactcaggatttgttcttacctttcttttccaatgttacttggttccatgctatacatgtctctgggaccaatggtactaagaggtttgataacc |
| | | ctgtcctaccatttaatgatggtgtttattttgcttccactgagaagtctaacataataagaggctggatctttggtactactttagattcgaagacccagtccctacttattgttaataac |
| | | gctactaatgttgttattaaagtctgtgaatttcaattttgtaatgatccatttttgggtgtttattaccacaaaaacaacaaaagttggatggaaagtgagttcagagtttattctagtgc |
| | | gaataattgcacttttgaatatgtctctcagccttttcttatggaccttgaaggaaaacagggtaatttcaaaaatcttagggaatttgtgtttaagaatattgatggttattttaaaatat |
| | | attctaagcacacgcctattaatttagtgcgtgatctccctcagggtttttcggctttagaaccattggtagatttgccaataggtattaacatcactaggtttcaaactttacttgcttta |
| | | catagaagttatttgactcctggtgattcttcttcaggttggacagctggtgctgcagcttattatgtgggttatcttcaacctaggacttttctattaaaatataatgaaaatggaaccat |
| | | tacagatgctgtagactgtgcacttgaccctctctcagaaacaaagtgtacgttgaaatccttcactgtagaaaaaggaatctatcaaacttctaactttagagtccaaccaacaga |
| | | atctattgttagatttcctaatattacaaacttgtgcccttttggtgaagtttttaacgccaccagatttgcatctgtttatgcttggaacaggaagagaatcagcaactgtgttgctgatt |
| | | attctgtcctatataattccgcatcattttccacttttaagtgttatggagtgtctcctactaaattaaatgatctctgctttactaatgtctatgcagattcatttgtaattagaggtgat |
| | | gaagtcagacaaatcgctccagggcaaactggaaagattgctgattataattataaattaccagatgattttacaggctgcgttatagcttggaattctaacaatcttgattctaaggttg |
| | | gtggtaattataattacctgtatagattgtttaggaagtctaatctcaaaccttttgagagagatatttcaactgaaatctatcaggccggtagcacaccttgtaatggtgttgaaggt |
| | | tttaattgttactttcctttacaatcatatggtttccaacccactaatggtgttggttaccaaccatacagagtagtagtactttcttttgaacttctacatgcaccagcaactgtttgtgg |
| | | acctaaaaagtctactaatttggttaaaaacaaatgtgtcaatttcaacttcaatggtttaacaggcacaggtgttcttactgagtctaacaaaaagtttctgcctttccaacaatttgg |
| | | cagagacattgctgacactactgatgctgtccgtgatccacagacacttgagattcttgacattacaccatgttcttttggtggtgtcagtgttataacaccaggaacaaatacttct |
| | | aaccaggttgctgttctttatcaggatgttaactgcacagaagtccctgttgctattcatgcagatcaacttactcctacttggcgtgtttattctacaggttctaatgtttttcaaacacg |
| | | tgcaggctgtttaataggggctgaacatgtcaacaactcatatgagtgtgacatacccattggtgcaggtatatgcgctagttatcagactcagactaattctcctggcagcgcc |
| | | agcagtgtagctagtcaatccatcattgcctacactatgtcacttggtgcagaaaattcagttgcttactctaataactctattgccatacccacaaattttactattagtgttaccaca |
| | | gaaattctaccagtgtctatgaccaagacatcagtagattgtacaatgtacatttgtggtgattcaactgaatgcagcaatcttttgttgcaatatggcagtttttgtacacaattaaa |
| | | ccgtgctttaactgggatagctgttgaacaagacaaaaacacccaagaagtttttgcacaagtcaaacaaatttacaaaacaccaccaattaaagattttggtggttttaattttt |
| | | cacaaatattaccagatccatcaaaaccaagcaagaggtcatttattgaagatctacttttcaacaaagtgacacttgcagatgctggcttcatcaaacaatatggtgattgcctt |
| | | ggtgatattgctgctagggacctcatttgtgcacaaaagtttaacggccttactgttttgccacctttgctcacagatgaaatgattgctcaatacacttctgcactgttagcgggtac |
| | | aatcacttctggttggacctttggtgcaggtgctgcattacaaataccatttgctatgcaaatggcttataggtttaatggtattggagttacacagaatgttctctatgagaaccaaa |
| | | aattgattgccaaccaatttaatagtgccattggcaaaattcaagactcactttcttccacagcaagtgcacttggaaaacttcaagatgtggtcaaccaaaatgcacaagcttt |
| | | aaacacgcttgttaaacaacttagctccaattttggtgcaatttcaagtgttttaaatgatatcctttcacgtcttgaccctcccgaggctgaagtgcaaattgataggttgatcacag |
| | | gcagacttcaaagtttgcagacatatgtgactcaacaattaattagagctgcagaaatcagagcttctgctaatcttgctgctactaaaatgtcagagtgtgtacttggacaatca |
| | | aaaagagttgatttttgtggaaagggctatcatcttatgtccttccctcagtcagcacctcatggtgtagtcttcttgcatgtgacttatgtccctgcacaagaaaagaacttcacaa |
| | | ctgctcctgccatttgtcatgatggaaaagcacactttcctcgtgaaggtgtctttgtttcaaatggcacacactggtttgtaacacaaaggaatttttatgaaccacaaatcattact |
| | | acagacaacacatttgtgtctggtaactgtgatgttgtaataggaattgtcaacaacacagtttatgatcctttgcaacctgaattagactcattcaaggaggagttagataaatat |
| | | tttaagaatcatacatcaccagatgttgatttaggtgacatctctggcattaatgcttcagttgtaaacattcaaaaagaaattgaccgcctcaatgaggttgccaagaatttaaat |
| | | gaatctctcatcgatctccaagaacttggaaagtatgagcagtatataaaatggccatggtacatttggctaggttttatagctggcttgattgccatagtaatggtgacaattatgc |
| | | tttgctgtatgaccagttgctgtagttgtctcaagggctgttgttcttgtggatcctgctgcaaatttgatgaagacgactctgagccagtgctcaaaggagtcaaattacattacac |
| | | aTAA |
| 22 | P1 | AGATTATCAAAAAGGATCTTCACCTAGATCCTTTTAAATTAAAAATGAAGTTTTAAATCAATCTAAAGTATATATGAGTAA |
| | | ACTTGGTCTGACAGTTATTAGAAAAATTCATCCAGCAGACGATAAAACGCAATACGCTGGCTATCCGGTGCCGCAATG |
| | | CCATACAGCACCAGAAAACGATCCGCCCATTCGCCGCCCAGTTCTTCCGCAATATCACGGGTGGCCAGCGCAATATC |
| | | CTGATAACGATCCGCCACGCCCAGACGGCCGCAATCAATAAAGCCGCTAAAACGGCCATTTTCCACCATAATGTTCG |
| | | GCAGGCACGCATCACCATGGGTCACCACCAGATCTTCGCCATCCGGCATGCTCGCTTTCAGACGCGCAAACAGCTCT |
| | | GCCGGTGCCAGGCCCTGATGTTCTTCATCCAGATCATCCTGATCCACCAGGCCCGCTTCCATACGGGTACGCGCACG |
| | | TTCAATACGATGTTTCGCCTGATGATCAAACGGACAGGTCGCCGGGTCCAGGGTATGCAGACGACGCATGGCATCCG |
| | | CCATAATGCTCACTTTTTCTGCCGGCGCCAGATGGCTAGACAGCAGATCCTGACCCGGCACTTCGCCCAGCAGCAGC |
| | | CAATCACGGCCCGCTTCGGTCACCACATCCAGCACCGCCGCACACGGAACACCGGTGGTGGCCAGCCAGCTCAGAC |
| | | GCGCCGCTTCATCCTGCAGCTCGTTCAGCGCACCGCTCAGATCGGTTTTCACAAACAGCACCGGACGACCCTGCGC |
| | | GCTCAGACGAAACACCGCCGCATCAGAGCAGCCAATGGTCTGCTGCGCCCAATCATAGCCAAACAGACGTTCCACCC |
| | | ACGCTGCCGGGCTACCCGCATGCAGGCCATCCTGTTCAATCATACTCTTCCTTTTTCAATATTATTGAAGCATTTATCA |
| | | GGGTTATTGTCTCATGAGCGGATACATATTTGAATGTATTTAGAAAAATAAACAAATAGGGGTTCCGCGCACATTTCCC |
| | | CGAAAAGTGCCACCTAAATTGTAAGCGTTAATATTTTGTTAAAATTCGCGTTAAATTTTTGTTAAATCAGCTCATTTTTTA |
| | | ACCAATAGGCCGAAATCGGCAAAATCCCTTATAAATCAAAAGAATAGACCGAGATAGGGTTGAGTGGCCGCTACAGG |
| | | GCGCTCCCATTCGCCATTCAGGCTGCGCAACTGTTGGGAAGGGCGTTTCGGTGCGGGCCTCTTCGCTATTACGCCA |
| | | GCTGGCGAAAGGGGGATGTGCTGCAAGGCGATTAAGTTGGGTAACGCCAGGGTTTTCCCAGTCACGACGTTGTAAAA |
| | | CGACGGCCAGTGAGCGCGACGTAATACGACTCACTATAGGGCGAATTGAAGGAAGGCCGTCAAGGCCGCATGGGAA |
| | | GCCCTCGACCGTCGATTGTCCACTGGTCAACAATAGATGACTTACAACTAATCGGAAGGTGCAGAGACTCGACGGGA |
| | | GCTACCCTAACGTCAAGACGAGGGTAAAGAGAGAGTCCAATTCTCAAAGCCAATAGGCAGTAGCGAAAGCTGCAAGA |
| | | GAATGAAAATCCGTTGACCTTAAACGGTCGTGTGGGTTCAAGTCCCTCCACCCCCACGCCGGAAACGCAATAGCCGA |
| | | AAAACAAAAAACAAAAAAAACAAAAAAAAAACCAAAAAAACAAAACACAacgttactggccgaagccgcttggaataaggccggtgtgcgttt |
| | | gtctatatgttattttccaccatattgccgtcttttggcaatgtgagggcccggaaacctggccctgtcttcttgacgagcattcctaggggtctttcccctctcgccaaaggaatgca |
| | | aggtctgttgaatgtcgtgaaggaagcagttcctctggaagcttcttgaagacaaacaacgtctgtagcgaccctttgcaggcagcggaaccccccacctggcgacaggtgc |
| | | ctctgcggccaaaagccacgtgtataagatacacctgcaaaggcggcacaaccccagtgccacgttgtgagttggatagttgtggaaagagtcaaatggctctcctcaagc |
| | | gtattcaacaaggggctgaaggatgcccagaaggtaccccattgtatgggatctgatctggggcctcggtgcacatgctttacatgtgtttagtcgaggttaaaaaacgtctag |
| | | gccccccgaaccacggggacgtggttttcctttgaaaaacacgatgataataGCCACCatgtttgtttttcttgttttattgccactagtctctagtcagtgtgttaatcttacaacc |
| | | agaactcaattaccccctgcatacactaattctttcacacgtggtgtttattaccctgacaaagttttcagatcctcagttttacattcaactcaggatttgttcttacctttcttttccaa |
| | | tgttacttggttccatgctatacatgtctctgggaccaatggtactaagaggtttgataaccctgtcctaccatttaatgatggtgtttattttgcttccactgagaagtctaacataataa |
| | | gaggctggatctttggtactactttagattcgaagacccagtccctacttattgttaataacgctactaatgttgttattaaagtctgtgaatttcaattttgtaatgatccatttttgggt |
| | | gtttattaccacaaaaacaacaaaagttggatggaaagtgagttcagagtttattctagtgcgaataattgcacttttgaatatgtctctcagccttttcttatggaccttgaaggaaaac |
| | | agggtaatttcaaaaatcttagggaatttgtgtttaagaatattgatggttattttaaaatatattctaagcacacgcctattaatttagtgcgtgatctccctcagggtttttcggcttta |
| | | gaaccattggtagatttgccaataggtattaacatcactaggtttcaaactttacttgctttacatagaagttatttgactcctggtgattcttcttcaggttggacagctggtgctgcagc |
| | | ttattatgtgggttatcttcaacctaggacttttctattaaaatataatgaaaatggaaccattacagatgctgtagactgtgcacttgaccctctctcagaaacaaagtgtacgttga |
| | | aatccttcactgtagaaaaaggaatctatcaaacttctaactttagagtccaaccaacagaatctattgttagatttcctaatattacaaacttgtgcccttttggtgaagtttttaacg |
| | | ccaccagatttgcatctgtttatgcttggaacaggaagagaatcagcaactgtgttgctgattattctgtcctatataattccgcatcattttccacttttaagtgttatggagtgtctcct |
| | | actaaattaaatgatctctgctttactaatgtctatgcagattcatttgtaattagaggtgatgaagtcagacaaatcgctccagggcaaactggaaagattgctgattataattata |
| | | aattaccagatgattttacaggctgcgttatagcttggaattctaacaatcttgattctaaggttggtggtaattataattacctgtatagattgtttaggaagtctaatctcaaacctttt |
| | | gagagagatatttcaactgaaatctatcaggccggtagcacaccttgtaatggtgttgaaggttttaattgttactttcctttacaatcatatggtttccaacccactaatggtgttggtt |
| | | accaaccatacagagtagtagtactttcttttgaacttctacatgcaccagcaactgtttgtggacctaaaaagtctactaatttggttaaaaacaaatgtgtcaatttcaacttcaat |
| | | ggtttaacaggcacaggtgttcttactgagtctaacaaaaagtttctgcctttccaacaatttggcagagacattgctgacactactgatgctgtccgtgatccacagacacttga |
| | | gattcttgacattacaccatgttcttttggtggtgtcagtgttataacaccaggaacaaatacttctaaccaggttgctgttctttatcaggatgttaactgcacagaagtccctgttgct |
| | | attcatgcagatcaacttactcctacttggcgtgtttattctacaggttctaatgtttttcaaacacgtgcaggctgtttaataggggctgaacatgtcaacaactcatatgagtgtgac |
| | | atacccattggtgcaggtatatgcgctagttatcagactcagactaattctcctggcagcgccagcagtgtagctagtcaatccatcattgcctacactatgtcacttggtgcaga |
| | | aaattcagttgcttactctaataactctattgccatacccacaaattttactattagtgttaccacagaaattctaccagtgtctatgaccaagacatcagtagattgtacaatgtaca |
| | | tttgtggtgattcaactgaatgcagcaatcttttgttgcaatatggcagtttttgtacacaattaaaccgtgctttaactgggatagctgttgaacaagacaaaaacacccaagaag |
| | | tttttgcacaagtcaaacaaatttacaaaacaccaccaattaaagattttggtggttttaatttttcacaaatattaccagatccatcaaaaccaagcaagaggtcatttattgaag |
| | | atctacttttcaacaaagtgacacttgcagatgctggcttcatcaaacaatatggtgattgccttggtgatattgctgctagggacctcatttgtgcacaaaagtttaacggccttact |
| | | gttttgccacctttgctcacagatgaaatgattgctcaatacacttctgcactgttagcgggtacaatcacttctggttggacctttggtgcaggtgctgcattacaaataccatttgct |
| | | atgcaaatggcttataggtttaatggtattggagttacacagaatgttctctatgagaaccaaaaattgattgccaaccaatttaatagtgccattggcaaaattcaagactcacttt |
| | | cttccacagcaagtgcacttggaaaacttcaagatgtggtcaaccaaaatgcacaagctttaaacacgcttgttaaacaacttagctccaattttggtgcaatttcaagtgttttaa |
| | | atgatatcctttcacgtcttgaccctcccgaggctgaagtgcaaattgataggttgatcacaggcagacttcaaagtttgcagacatatgtgactcaacaattaattagagctgca |
| | | gaaatcagagcttctgctaatcttgctgctactaaaatgtcagagtgtgtacttggacaatcaaaaagagttgatttttgtggaaagggctatcatcttatgtccttccctcagtcag |
| | | cacctcatggtgtagtcttcttgcatgtgacttatgtccctgcacaagaaaagaacttcacaactgctcctgccatttgtcatgatggaaaagcacactttcctcgtgaaggtgtcttt |
| | | gtttcaaatggcacacactggtttgtaacacaaaggaatttttatgaaccacaaatcattactacagacaacacatttgtgtctggtaactgtgatgttgtaataggaattgtcaac |
| | | aacacagtttatgatcctttgcaacctgaattagactcattcaaggaggagttagataaatattttaagaatcatacatcaccagatgttgatttaggtgacatctctggcattaatg |
| | | cttcagttgtaaacattcaaaaagaaattgaccgcctcaatgaggttgccaagaatttaaatgaatctctcatcgatctccaagaacttggaaagtatgagcagtatataaaatg |
| | | gccatggtacatttggctaggttttatagctggcttgattgccatagtaatggtgacaattatgctttgctgtTAAAAAAAACAAAAAACAAAACGGCTATTATG |
| | | CGTTACCGGCGAGACGCTACGGACTTAAATAATTGAGCCTTAAAGAAGAAATTCTTTAAGTGGATGCTCTCAAACTCA |
| | | GGGAAACCTAAATCTAGTTATAGACAAGGCAATCCTGAGCCAAGCCGAAGTAGTAATTAGTAAGACCAGTGGACAATC |
| | | GACGGATAACAGCATATCTAGCTGGGCCTCATGGGCCTTCCTTTCACTGCCCGCTTTCCAGTCGGGAAACCTGTCGT |
| | | GCCAGCTGCATTAACATGGTCATAGCTGTTTCCTTGCGTATTGGGCGCTCTCCGCTTCCTCGCTCACTGACTCGCTGC |
| | | GCTCGGTCGTTCGGGTAAAGCCTGGGGTGCCTAATGAGCAAAAGGCCAGCAAAAGGCCAGGAACCGTAAAAAGGCC |
| | | GCGTTGCTGGCGTTTTTCCATAGGCTCCGCCCCCCTGACGAGCATCACAAAAATCGACGCTCAAGTCAGAGGTGGCG |
| | | AAACCCGACAGGACTATAAAGATACCAGGCGTTTCCCCCTGGAAGCTCCCTCGTGCGCTCTCCTGTTCCGACCCTGC |
| | | CGCTTACCGGATACCTGTCCGCCTTTCTCCCTTCGGGAAGCGTGGCGCTTTCTCATAGCTCACGCTGTAGGTATCTCA |
| | | GTTCGGTGTAGGTCGTTCGCTCCAAGCTGGGCTGTGTGCACGAACCCCCCGTTCAGCCCGACCGCTGCGCCTTATC |
| | | CGGTAACTATCGTCTTGAGTCCAACCCGGTAAGACACGACTTATCGCCACTGGCAGCAGCCACTGGTAACAGGATTA |
| | | GCAGAGCGAGGTATGTAGGCGGTGCTACAGAGTTCTTGAAGTGGTGGCCTAACTACGGCTACACTAGAAGAACAGTA |
| | | TTTGGTATCTGCGCTCTGCTGAAGCCAGTTACCTTCGGAAAAAGAGTTGGTAGCTCTTGATCCGGCAAACAAACCACC |
| | | GCTGGTAGCGGTGGTTTTTTTGTTTGCAAGCAGCAGATTACGCGCAGAAAAAAAGGATCTCAAGAAGATCCTTTGATC |
| | | TTTTCTACGGGGTCTGACGCTCAGTGGAACGAAAACTCACGTTAAGGGATTTTGGTCATG |
| 23 | ORF11 | atgtttgtttttcttgttttattgccactagtctctagtcagtgtgttaatcttacaaccagaactcaattaccccctgcatacactaattctttcacacgtggtgtttattaccctgacaa |
| | | agttttcagatcctcagttttacattcaactcaggatttgttcttacctttcttttccaatgttacttggttccatgctatacatgtctctgggaccaatggtactaagaggtttgataacc |
| | | ctgtcctaccatttaatgatggtgtttattttgcttccactgagaagtctaacataataagaggctggatctttggtactactttagattcgaagacccagtccctacttattgttaataac |
| | | gctactaatgttgttattaaagtctgtgaatttcaattttgtaatgatccatttttgggtgtttattaccacaaaaacaacaaaagttggatggaaagtgagttcagagtttattctagtgc |
| | | gaataattgcacttttgaatatgtctctcagccttttcttatggaccttgaaggaaaacagggtaatttcaaaaatcttagggaatttgtgtttaagaatattgatggttattttaaaatat |
| | | attctaagcacacgcctattaatttagtgcgtgatctccctcagggtttttcggctttagaaccattggtagatttgccaataggtattaacatcactaggtttcaaactttacttgcttta |
| | | catagaagttatttgactcctggtgattcttcttcaggttggacagctggtgctgcagcttattatgtgggttatcttcaacctaggacttttctattaaaatataatgaaaatggaaccat |
| | | tacagatgctgtagactgtgcacttgaccctctctcagaaacaaagtgtacgttgaaatccttcactgtagaaaaaggaatctatcaaacttctaactttagagtccaaccaacaga |
| | | atctattgttagatttcctaatattacaaacttgtgcccttttggtgaagtttttaacgccaccagatttgcatctgtttatgcttggaacaggaagagaatcagcaactgtgttgctgatt |
| | | attctgtcctatataattccgcatcattttccacttttaagtgttatggagtgtctcctactaaattaaatgatctctgctttactaatgtctatgcagattcatttgtaattagaggtgat |
| | | gaagtcagacaaatcgctccagggcaaactggaaagattgctgattataattataaattaccagatgattttacaggctgcgttatagcttggaattctaacaatcttgattctaaggttg |
| | | gtggtaattataattacctgtatagattgtttaggaagtctaatctcaaaccttttgagagagatatttcaactgaaatctatcaggccggtagcacaccttgtaatggtgttgaaggt |
| | | tttaattgttactttcctttacaatcatatggtttccaacccactaatggtgttggttaccaaccatacagagtagtagtactttcttttgaacttctacatgcaccagcaactgtttgtgg |
| | | acctaaaaagtctactaatttggttaaaaacaaatgtgtcaatttcaacttcaatggtttaacaggcacaggtgttcttactgagtctaacaaaaagtttctgcctttccaacaatttgg |
| | | cagagacattgctgacactactgatgctgtccgtgatccacagacacttgagattcttgacattacaccatgttcttttggtggtgtcagtgttataacaccaggaacaaatacttct |
| | | aaccaggttgctgttctttatcaggatgttaactgcacagaagtccctgttgctattcatgcagatcaacttactcctacttggcgtgtttattctacaggttctaatgtttttcaaacacg |
| | | tgcaggctgtttaataggggctgaacatgtcaacaactcatatgagtgtgacatacccattggtgcaggtatatgcgctagttatcagactcagactaattctcctggcagcgcc |
| | | agcagtgtagctagtcaatccatcattgcctacactatgtcacttggtgcagaaaattcagttgcttactctaataactctattgccatacccacaaattttactattagtgttaccaca |
| | | gaaattctaccagtgtctatgaccaagacatcagtagattgtacaatgtacatttgtggtgattcaactgaatgcagcaatcttttgttgcaatatggcagtttttgtacacaattaaa |
| | | ccgtgctttaactgggatagctgttgaacaagacaaaaacacccaagaagtttttgcacaagtcaaacaaatttacaaaacaccaccaattaaagattttggtggttttaattttt |
| | | cacaaatattaccagatccatcaaaaccaagcaagaggtcatttattgaagatctacttttcaacaaagtgacacttgcagatgctggcttcatcaaacaatatggtgattgcctt |
| | | ggtgatattgctgctagggacctcatttgtgcacaaaagtttaacggccttactgttttgccacctttgctcacagatgaaatgattgctcaatacacttctgcactgttagcgggtac |
| | | aatcacttctggttggacctttggtgcaggtgctgcattacaaataccatttgctatgcaaatggcttataggtttaatggtattggagttacacagaatgttctctatgagaaccaaa |
| | | aattgattgccaaccaatttaatagtgccattggcaaaattcaagactcactttcttccacagcaagtgcacttggaaaacttcaagatgtggtcaaccaaaatgcacaagcttt |
| | | aaacacgcttgttaaacaacttagctccaattttggtgcaatttcaagtgttttaaatgatatcctttcacgtcttgaccctcccgaggctgaagtgcaaattgataggttgatcacag |
| | | gcagacttcaaagtttgcagacatatgtgactcaacaattaattagagctgcagaaatcagagcttctgctaatcttgctgctactaaaatgtcagagtgtgtacttggacaatca |
| | | aaaagagttgatttttgtggaaagggctatcatcttatgtccttccctcagtcagcacctcatggtgtagtcttcttgcatgtgacttatgtccctgcacaagaaaagaacttcacaa |
| | | ctgctcctgccatttgtcatgatggaaaagcacactttcctcgtgaaggtgtctttgtttcaaatggcacacactggtttgtaacacaaaggaatttttatgaaccacaaatcattact |
| | | acagacaacacatttgtgtctggtaactgtgatgttgtaataggaattgtcaacaacacagtttatgatcctttgcaacctgaattagactcattcaaggaggagttagataaatat |
| | | tttaagaatcatacatcaccagatgttgatttaggtgacatctctggcattaatgcttcagttgtaaacattcaaaaagaaattgaccgcctcaatgaggttgccaagaatttaaat |
| | | gaatctctcatcgatctccaagaacttggaaagtatgagcagtatataaaatggccatggtacatttggctaggttttatagctggcttgattgccatagtaatggtgacaattatgc |
| | | tttgctgtTAA |
| 24 | P13 | AGATTATCAAAAAGGATCTTCACCTAGATCCTTTTAAATTAAAAATGAAGTTTTAAATCAATCTAAAGTATATATGAGTAA |
| | | ACTTGGTCTGACAGTTATTAGAAAAATTCATCCAGCAGACGATAAAACGCAATACGCTGGCTATCCGGTGCCGCAATG |
| | | CCATACAGCACCAGAAAACGATCCGCCCATTCGCCGCCCAGTTCTTCCGCAATATCACGGGTGGCCAGCGCAATATC |
| | | CTGATAACGATCCGCCACGCCCAGACGGCCGCAATCAATAAAGCCGCTAAAACGGCCATTTTCCACCATAATGTTCG |
| | | GCAGGCACGCATCACCATGGGTCACCACCAGATCTTCGCCATCCGGCATGCTCGCTTTCAGACGCGCAAACAGCTCT |
| | | GCCGGTGCCAGGCCCTGATGTTCTTCATCCAGATCATCCTGATCCACCAGGCCCGCTTCCATACGGGTACGCGCACG |
| | | TTCAATACGATGTTTCGCCTGATGATCAAACGGACAGGTCGCCGGGTCCAGGGTATGCAGACGACGCATGGCATCCG |
| | | CCATAATGCTCACTTTTTCTGCCGGCGCCAGATGGCTAGACAGCAGATCCTGACCCGGCACTTCGCCCAGCAGCAGC |
| | | CAATCACGGCCCGCTTCGGTCACCACATCCAGCACCGCCGCACACGGAACACCGGTGGTGGCCAGCCAGCTCAGAC |
| | | GCGCCGCTTCATCCTGCAGCTCGTTCAGCGCACCGCTCAGATCGGTTTTCACAAACAGCACCGGACGACCCTGCGC |
| | | GCTCAGACGAAACACCGCCGCATCAGAGCAGCCAATGGTCTGCTGCGCCCAATCATAGCCAAACAGACGTTCCACCC |
| | | ACGCTGCCGGGCTACCCGCATGCAGGCCATCCTGTTCAATCATACTCTTCCTTTTTCAATATTATTGAAGCATTTATCA |
| | | GGGTTATTGTCTCATGAGCGGATACATATTTGAATGTATTTAGAAAAATAAACAAATAGGGGTTCCGCGCACATTTCCC |
| | | CGAAAAGTGCCACCTAAATTGTAAGCGTTAATATTTTGTTAAAATTCGCGTTAAATTTTTGTTAAATCAGCTCATTTTTTA |
| | | ACCAATAGGCCGAAATCGGCAAAATCCCTTATAAATCAAAAGAATAGACCGAGATAGGGTTGAGTGGCCGCTACAGG |
| | | GCGCTCCCATTCGCCATTCAGGCTGCGCAACTGTTGGGAAGGGCGTTTCGGTGCGGGCCTCTTCGCTATTACGCCA |
| | | GCTGGCGAAAGGGGGATGTGCTGCAAGGCGATTAAGTTGGGTAACGCCAGGGTTTTCCCAGTCACGACGTTGTAAAA |
| | | CGACGGCCAGTGAGCGCGACGTAATACGACTCACTATAGGGCGAATTGAAGGAAGGCCGTCAAGGCCGCATGGGAA |
| | | GCCCTCGACCGTCGATTGTCCACTGGTCAACAATAGATGACTTACAACTAATCGGAAGGTGCAGAGACTCGACGGGA |
| | | GCTACCCTAACGTCAAGACGAGGGTAAAGAGAGAGTCCAATTCTCAAAGCCAATAGGCAGTAGCGAAAGCTGCAAGA |
| | | GAATGAAAATCCGTTGACCTTAAACGGTCGTGTGGGTTCAAGTCCCTCCACCCCCACGCCGGAAACGCAATAGCCGA |
| | | AAAACAAAAAACAAAAAAAACAAAAAAAAAACCAAAAAAACAAAACACAacgttactggccgaagccgcttggaataaggccggtgtgcgttt |
| | | gtctatatgttattttccaccatattgccgtcttttggcaatgtgagggcccggaaacctggccctgtcttcttgacgagcattcctaggggtctttcccctctcgccaaaggaatgca |
| | | aggtctgttgaatgtcgtgaaggaagcagttcctctggaagcttcttgaagacaaacaacgtctgtagcgaccctttgcaggcagcggaaccccccacctggcgacaggtgc |
| | | ctctgcggccaaaagccacgtgtataagatacacctgcaaaggcggcacaaccccagtgccacgttgtgagttggatagttgtggaaagagtcaaatggctctcctcaagc |
| | | gtattcaacaaggggctgaaggatgcccagaaggtaccccattgtatgggatctgatctggggcctcggtgcacatgctttacatgtgtttagtcgaggttaaaaaacgtctag |
| | | gccccccgaaccacggggacgtggttttcctttgaaaaacacgatgataataGCCACCatgtttgtttttcttgttttattgccactagtctctagtcagtgtagagtccaaccaa |
| | | cagaatctattgttagatttcctaatattacaaacttgtgcccttttggtgaagtttttaacgccaccagatttgcatctgtttatgcttggaacaggaagagaatcagcaactgtgttg |
| | | ctgattattctgtcctatataattccgcatcattttccacttttaagtgttatggagtgtctcctactaaattaaatgatctctgctttactaatgtctatgcagattcatttgtaattaga |
| | | ggtgatgaagtcagacaaatcgctccagggcaaactggaaagattgctgattataattataaattaccagatgattttacaggctgcgttatagcttggaattctaacaatcttgattcta |
| | | aggttggtggtaattataattacctgtatagattgtttaggaagtctaatctcaaaccttttgagagagatatttcaactgaaatctatcaggccggtagcacaccttgtaatggtgtt |
| | | gaaggttttaattgttactttcctttacaatcatatggtttccaacccactaatggtgttggttaccaaccatacagagtagtagtactttcttttgaacttctacatgcaccagcaactgt |
| | | ttgtggacctaaaaagtctactaatttggttaaaaacaaatgtgtcaatttcTAAAAAAAACAAAAAACAAAACGGCTATTATGCGTTACCGGCGA |
| | | GACGCTACGGACTTAAATAATTGAGCCTTAAAGAAGAAATTCTTTAAGTGGATGCTCTCAAACTCAGGGAAACCTAAAT |
| | | CTAGTTATAGACAAGGCAATCCTGAGCCAAGCCGAAGTAGTAATTAGTAAGACCAGTGGACAATCGACGGATAACAGC |
| | | ATATCTAGCTGGGCCTCATGGGCCTTCCTTTCACTGCCCGCTTTCCAGTCGGGAAACCTGTCGTGCCAGCTGCATTAA |
| | | CATGGTCATAGCTGTTTCCTTGCGTATTGGGCGCTCTCCGCTTCCTCGCTCACTGACTCGCTGCGCTCGGTCGTTCG |
| | | GGTAAAGCCTGGGGTGCCTAATGAGCAAAAGGCCAGCAAAAGGCCAGGAACCGTAAAAAGGCCGCGTTGCTGGCGT |
| | | TTTTCCATAGGCTCCGCCCCCCTGACGAGCATCACAAAAATCGACGCTCAAGTCAGAGGTGGCGAAACCCGACAGGA |
| | | CTATAAAGATACCAGGCGTTTCCCCCTGGAAGCTCCCTCGTGCGCTCTCCTGTTCCGACCCTGCCGCTTACCGGATA |
| | | CCTGTCCGCCTTTCTCCCTTCGGGAAGCGTGGCGCTTTCTCATAGCTCACGCTGTAGGTATCTCAGTTCGGTGTAGGT |
| | | CGTTCGCTCCAAGCTGGGCTGTGTGCACGAACCCCCCGTTCAGCCCGACCGCTGCGCCTTATCCGGTAACTATCGTC |
| | | TTGAGTCCAACCCGGTAAGACACGACTTATCGCCACTGGCAGCAGCCACTGGTAACAGGATTAGCAGAGCGAGGTAT |
| | | GTAGGCGGTGCTACAGAGTTCTTGAAGTGGTGGCCTAACTACGGCTACACTAGAAGAACAGTATTTGGTATCTGCGCT |
| | | CTGCTGAAGCCAGTTACCTTCGGAAAAAGAGTTGGTAGCTCTTGATCCGGCAAACAAACCACCGCTGGTAGCGGTGG |
| | | TTTTTTTGTTTGCAAGCAGCAGATTACGCGCAGAAAAAAAGGATCTCAAGAAGATCCTTTGATCTTTTCTACGGGGTCT |
| | | GACGCTCAGTGGAACGAAAACTCACGTTAAGGGATTTTGGTCATG |
| 25 | ORF13 | atgtttgtttttcttgttttattgccactagtctctagtcagtgtagagtccaaccaacagaatctattgttagatttcctaatattacaaacttgtgcccttttggtgaagtttttaacgc |
| | | caccagatttgcatctgtttatgcttggaacaggaagagaatcagcaactgtgttgctgattattctgtcctatataattccgcatcattttccacttttaagtgttatggagtgtctccta |
| | | ctaaattaaatgatctctgctttactaatgtctatgcagattcatttgtaattagaggtgatgaagtcagacaaatcgctccagggcaaactggaaagattgctgattataattataaatt |
| | | accagatgattttacaggctgcgttatagcttggaattctaacaatcttgattctaaggttggtggtaattataattacctgtatagattgtttaggaagtctaatctcaaaccttttgag |
| | | agagatatttcaactgaaatctatcaggccggtagcacaccttgtaatggtgttgaaggttttaattgttactttcctttacaatcatatggtttccaacccactaatggtgttggttacc |
| | | aaccatacagagtagtagtactttcttttgaacttctacatgcaccagcaactgtttgtggacctaaaaagtctactaatttggttaaaaacaaatgtgtcaatttcTAA |
| 26 | ORF33 | atgtttgtttttcttgttttattgccactagtctctagtcagtgtagagtccaaccaacagaatctattgttagatttcctaatattacaaacttgtgcccttttggtgaagtttttaacgc |
| | | caccagatttgcatccgtgtatgcttggaacaggaagagaatcagcaactgtgttgctgattattctgtcctatataattccgcatcattttccacttttaagtgttatggagtgtctccta |
| | | ctaaattaaatgatctctgctttactaatgtctatgcagattcatttgtaattagaggtgatgaagtcagacaaatcgctccagggcaaactggaaagattgctgattataattataaatt |
| | | accagatgattttacaggctgcgttatagcttggaattctaacaatcttgattctaaggttggtggtaattataattacctgtatagattgtttaggaagtctaatctcaaaccttttgag |
| | | agagatatttcaactgaaatctatcaggccggtagcacaccttgtaatggtgttgaaggttttaattgttactttcctttacaatcatatggtttccaacccactaatggtgttggttacc |
| | | aaccatacagagtagtagtactttcttttgaacttctacatgcaccagcaactgtttgtggacctaaaaagtctactaatttggttaaaaacaaatgtgtcaatttcTAA |
| 27 | ORF35 | atgtttgtttttcttgttttattgccactagtctctagtcagtgtgttaatcttacaaccagaactcaattaccccctgcatacactaattctttcacacgtggtgtttattaccctgacaa |
| | | agttttcagatcctcagttttacattcaactcaggacttgttcttacctttcttttccaatgttacttggttccatgctatacatgtctctgggaccaatggtactaagaggtttgataacc |
| | | ctgtcctaccatttaatgatggtgtttattttgcttccactgagaagtctaacataataagaggctggatttttggtactactttagattcgaagacccagtccctacttattgttaataac |
| | | gctactaatgttgttattaaagtctgtgaatttcaattttgtaatgatccatttttgggtgtttattaccacaaaaacaacaaaagttggatggaaagtgagttcagagtttattctagtgc |
| | | gaataattgcacttttgaatatgtctctcagccttttcttatggaccttgaaggaaaacagggtaatttcaaaaatcttagggaatttgtgtttaagaatattgatggttattttaaaatat |
| | | attctaagcacacgcctattaatttagtgcgtgatctccctcagggtttttcggctttagaaccattggtagatttgccaataggtattaacatcactaggtttcaaactttacttgcttta |
| | | catagaagttatttgactcctggtgattcttcttcaggttggacagctggtgctgcagcttattatgtgggttatcttcaacctaggacttttctattaaaatataatgaaaatggaaccat |
| | | tacagatgctgtagactgtgcacttgaccctctctcagaaacaaagtgtacgttgaaatccttcactgtagaaaaaggaatctatcaaacttctaactttagagtccaaccaacaga |
| | | atctattgttagatttcctaatattacaaacttgtgcccttttggtgaagtttttaacgccaccagatttgcatccgtgtatgcttggaacaggaagagaatcagcaactgtgttgctga |
| | | ttattctgtcctatataattccgcatcattttccacttttaagtgttatggagtgtctcctactaaattaaatgatctctgctttactaatgtctatgcagattcatttgtaattagaggtg |
| | | atgaagtcagacaaatcgctccagggcaaactggaaagattgctgattataattataaattaccagatgattttacaggctgcgttatagcttggaattctaacaatcttgattctaaggtt |
| | | ggtggtaattataattacctgtatagattgtttaggaagtctaatctcaaaccttttgagagagatatttcaactgaaatctatcaggccggtagcacaccttgtaatggtgttgaag |
| | | gttttaattgttactttcctttacaatcatatggtttccaacccactaatggtgttggttaccaaccatacagagtagtagtactttcttttgaacttctacatgcaccagcaactgtttgt |
| | | ggacctaaaaagtctactaatttggttaaaaacaaatgtgtcaatttcaacttcaatggtttaacaggcacaggtgttcttactgagtctaacaaaaagtttctgcctttccaacaattt |
| | | ggcagagacattgctgacactactgatgctgtccgtgatccacagacacttgagattcttgacattacaccatgttcttttggtggtgtcagtgttataacaccaggaacaaatact |
| | | tctaaccaggttgctgttctttatcaggatgttaactgcacagaagtccctgttgctattcatgcagatcaacttactcctacttggcgtgtttattctacaggttctaatgtttttcaaac |
| | | acgtgcaggctgtttaataggggctgaacatgtcaacaactcatatgagtgtgacatacccattggtgcaggtatatgcgctagttatcagactcagactaattctcctggcagcg |
| | | ccagcagtgtagctagtcaatccatcattgcctacactatgtcacttggtgcagaaaattcagttgcttactctaataactctattgccatacccacaaattttactattagtgttacca |
| | | cagaaattctaccagtgtctatgaccaagacatcagtagattgtacaatgtacatttgtggtgattcaactgaatgcagcaatcttttgttgcaatatggcagtttttgtacacaatta |
| | | aaccgtgctttaactggaatagctgttgaacaagacaaaaacacccaagaagtttttgcacaagtcaaacaaatttacaaaacaccaccaattaaagattttggtggttttaatt |
| | | tttcacaaatattaccagatccatcaaaaccaagcaagaggtcatttattgaagatctacttttcaacaaagtgacacttgcagatgctggcttcatcaaacaatatggtgattgc |
| | | cttggtgatattgctgctagggacctcatttgtgcacaaaagtttaacggccttactgttttgccacctttgctcacagatgaaatgattgctcaatacacttctgcactgttagcgggt |
| | | acaatcacttctggttggacctttggtgcaggtgctgcattacaaataccatttgctatgcaaatggcttataggtttaatggtattggagttacacagaatgttctctatgagaacca |
| | | aaaattgattgccaaccaatttaatagtgctattggcaaaattcaagactcactttcttccacagcaagtgcacttggaaaacttcaagatgtggtcaaccaaaatgcacaagct |
| | | ttaaacacgcttgttaaacaacttagctccaattttggtgcaatttcaagtgttttaaatgatatcctttcacgtcttgaccctcccgaggctgaagtgcaaattgataggttgatcaca |
| | | ggcagacttcaaagtttgcagacatatgtgactcaacaattaattagagctgcagaaatcagagcttctgctaatcttgctgctactaaaatgtcagagtgtgtacttggacaatc |
| | | aaaaagagttgatttttgtggaaagggctatcatcttatgtccttccctcagtcagcacctcatggtgtagtcttcttgcatgtgacttatgtccctgcacaagaaaagaacttcaca |
| | | actgctcctgccatttgtcatgatggaaaagcacactttcctcgtgaaggtgtctttgtttcaaatggcacacactggtttgtaacacaaaggaatttttatgaaccacaaatcatta |
| | | ctacagacaacacatttgtgtctggtaactgtgatgttgtaataggaattgtcaacaacacagtttatgatcctttgcaacctgaattagactcattcaaggaggagttagataaat |
| | | attttaagaatcatacatcaccagatgttgatttaggtgacatctctggcattaatgcttcagttgtaaacattcaaaaagaaattgaccgcctcaatgaggttgccaagaatttaa |
| | | atgaatctctcatcgatctccaagaacttggaaagtatgagcagtatataaaatggccatggtacatttggctaggttttatagctggcttgattgccatagtaatggtgacaattat |
| | | gctttgctgtatgaccagttgctgtagttgtctcaagggctgttgttcttgtggatcctgctgcaaatttgatgaagacgactctgagccagtgctcaaaggagtcaaattacattac |
| | | acaTAA |
| 28 | ORF36 | atgtttgtttttcttgttttattgccactagtctctagtcagtgtgttaatcttacaaccagaactcaattaccccctgcatacactaattctttcacacgtggtgtttattaccctgacaa |
| | | agttttcagatcctcagttttacattcaactcaggatttgttcttacctttcttttccaatgttacttggttccatgctatacatgtctctgggaccaatggtactaagaggtttgataacc |
| | | ctgtcctaccatttaatgatggtgtttattttgcttccactgagaagtctaacataataagaggctggatctttggtactactttagattcgaagacccagtccctacttattgttaataac |
| | | gctactaatgttgttattaaagtctgtgaatttcaattttgtaatgatccatttttgggtgtttattaccacaaaaacaacaaaagttggatggaaagtgagttcagagtttattctagtgc |
| | | gaataattgcacttttgaatatgtctctcagccttttcttatggaccttgaaggaaaacagggtaatttcaaaaatcttagggaatttgtgtttaagaatattgatggttattttaaaatat |
| | | attctaagcacacgcctattaatttagtgcgtgatctccctcagggtttttcggctttagaaccattggtagatttgccaataggtattaacatcactaggtttcaaactttacttgcttta |
| | | catagaagttatttgactcctggtgattcttcttcaggttggacagctggtgctgcagcttattatgtgggttatcttcaacctaggacttttctattaaaatataatgaaaatggaaccat |
| | | tacagatgctgtagactgtgcacttgaccctctctcagaaacaaagtgtacgttgaaatccttcactgtagaaaaaggaatctatcaaacttctaactttagagtccaaccaacaga |
| | | atctattgttagatttcctaatattacaaacttgtgcccttttggtgaagtttttaacgccaccagatttgcatccgtgtatgcttggaacaggaagagaatcagcaactgtgttgctga |
| | | ttattctgtcctatataattccgcatcattttccacttttaagtgttatggagtgtctcctactaaattaaatgatctctgctttactaatgtctatgcagattcatttgtaattagaggtg |
| | | atgaagtcagacaaatcgctccagggcaaactggaaagattgctgattataattataaattaccagatgattttacaggctgcgttatagcttggaattctaacaatcttgattctaaggtt |
| | | ggtggtaattataattacctgtatagattgtttaggaagtctaatctcaaaccttttgagagagatatttcaactgaaatctatcaggccggtagcacaccttgtaatggtgttgaag |
| | | gttttaattgttactttcctttacaatcatatggtttccaacccactaatggtgttggttaccaaccatacagagtagtagtactttcttttgaacttctacatgcaccagcaactgtttgt |
| | | ggacctaaaaagtctactaatttggttaaaaacaaatgtgtcaatttcaacttcaatggtttaacaggcacaggtgttcttactgagtctaacaaaaagtttctgcctttccaacaattt |
| | | ggcagagacattgctgacactactgatgctgtccgtgatccacagacacttgagattcttgacattacaccatgttcttttggtggtgtcagtgttataacaccaggaacaaatact |
| | | tctaaccaggttgctgttctttatcaggatgttaactgcacagaagtccctgttgctattcatgcagatcaacttactcctacttggcgtgtttattctacaggttctaatgtttttcaaac |
| | | acgtgcaggctgtttaataggggctgaacatgtcaacaactcatatgagtgtgacatacccattggtgcaggtatatgcgctagttatcagactcagactaattctcctggcagcg |
| | | ccagcagtgtagctagtcaatccatcattgcctacactatgtcacttggtgcagaaaattcagttgcttactctaataactctattgccatacccacaaattttactattagtgttacca |
| | | cagaaattctaccagtgtctatgaccaagacatcagtagattgtacaatgtacatttgtggtgattcaactgaatgcagcaatcttttgttgcaatatggcagtttttgtacacaatta |
| | | aaccgtgctttaactgggatagctgttgaacaagacaaaaacacccaagaagtttttgcacaagtcaaacaaatttacaaaacaccaccaattaaagattttggtggttttaatt |
| | | tttcacaaatattaccagatccatcaaaaccaagcaagaggtcatttattgaagatctacttttcaacaaagtgacacttgcagatgctggcttcatcaaacaatatggtgattgc |
| | | cttggtgatattgctgctagggacctcatttgtgcacaaaagtttaacggccttactgttttgccacctttgctcacagatgaaatgattgctcaatacacttctgcactgttagcgggt |
| | | acaatcacttctggttggacctttggtgcaggtgctgcattacaaataccatttgctatgcaaatggcttataggtttaatggtattggagttacacagaatgttctctatgagaacca |
| | | aaaattgattgccaaccaatttaatagtgccattggcaaaattcaagactcactttcttccacagcaagtgcacttggaaaacttcaagatgtggtcaaccaaaatgcacaagc |
| | | tttaaacacgcttgttaaacaacttagctccaattttggtgcaatttcaagtgttttaaatgatatcctttcacgtcttgaccctcccgaggctgaagtgcaaattgataggttgatcac |
| | | aggcagacttcaaagtttgcagacatatgtgactcaacaattaattagagctgcagaaatcagagcttctgctaatcttgctgctactaaaatgtcagagtgtgtacttggacaat |
| | | caaaaagagttgatttttgtggaaagggctatcatcttatgtccttccctcagtcagcacctcatggtgtagtcttcttgcatgtgacttatgtccctgcacaagaaaagaacttcac |
| | | aactgctcctgccatttgtcatgatggaaaagcacactttcctcgtgaaggtgtctttgtttcaaatggcacacactggtttgtaacacaaaggaatttttatgaaccacaaatcatt |
| | | actacagacaacacatttgtgtctggtaactgtgatgttgtaataggaattgtcaacaacacagtttatgatcctttgcaacctgaattagactcattcaaggaggagttagataaa |
| | | tattttaagaatcatacatcaccagatgttgatttaggtgacatctctggcattaatgcttcagttgtaaacattcaaaaagaaattgaccgcctcaatgaggttgccaagaatttaa |
| | | atgaatctctcatcgatctccaagaacttggaaagtatgagcagtatataaaatggccatggtacatttggctaggttttatagctggcttgattgccatagtaatggtgacaattat |
| | | gctttgctgtatgaccagttgctgtagttgtctcaagggctgttgttcttgtggatcctgctgcaaatttgatgaagacgactctgagccagtgctcaaaggagtcaaattacattac |
| | | acaTAA |
| 29 | ORF39 | ATGTTCGTTTTCCTTGTTTTACTGCCCCTCGTGTCTTCACAGTGTGTGAACCTCACCACTCGCACACAGCTACCTCCCG |
| | | CGTACACTAATTCATTTACCAGGGGCGTCTATTATCCTGATAAGGTGTTCCGGAGTTCAGTGTTGCATAGCACTCAAG |
| | | ACCTGTTCCTGCCCTTCTTCTCCAATGTCACTTGGTTTCATGCGATACATGTGTCTGGTACCAACGGAACGAAGAGATT |
| | | TGATAACCCCGTACTGCCATTCAATGATGGCGTATACTTTGCTTCGACTGAAAAATCCAACATCATCAGGGGCTGGAT |
| | | TTTTGGTACAACGCTTGATTCCAAGACCCAGTCCCTCCTTATTGTGAACAATGCGACCAACGTCGTGATAAAGGTCTG |
| | | TGAGTTTCAGTTCTGCAATGACCCATTCCTTGGAGTGTATTATCACAAGAACAACAAATCGTGGATGGAGTCAGAGTTT |
| | | AGGGTGTACAGCAGCGCTAACAACTGTACATTTGAGTATGTGAGTCAGCCGTTTCTGATGGATCTTGAGGGCAAACAG |
| | | GGCAATTTTAAGAATCTCAGAGAATTTGTGTTCAAGAACATTGATGGTTACTTTAAGATCTATAGCAAACATACGCCAAT |
| | | CAACTTGGTTCGTGATCTGCCACAGGGATTTAGCGCACTGGAACCTCTCGTTGACTTGCCCATAGGTATTAACATCAC |
| | | CAGGTTCCAGACGCTCTTGGCATTACACCGTAGTTATCTGACCCCCGGGGACTCCAGTTCCGGATGGACTGCAGGAG |
| | | CCGCTGCCTACTATGTGGGTTACCTCCAGCCCAGGACCTTTCTTTTGAAATATAACGAGAACGGCACAATCACTGATG |
| | | CTGTGGACTGCGCATTGGATCCTTTGTCAGAGACTAAGTGCACTCTGAAGTCATTCACAGTCGAGAAAGGCATTTACC |
| | | AGACGTCTAACTTCAGGGTTCAGCCTACTGAGTCCATCGTGAGATTCCCAAACATCACAAATCTTTGTCCCTTCGGTG |
| | | AGGTATTCAATGCGACACGATTTGCCTCAGTGTACGCGTGGAATCGGAAGAGGATCTCCAATTGCGTGGCCGACTAC |
| | | TCCGTCTTATACAACTCAGCTAGCTTTTCAACATTCAAGTGCTATGGCGTGAGCCCTACCAAGCTCAATGACCTGTGCT |
| | | TCACTAATGTGTATGCCGACTCTTTTGTCATTCGCGGCGACGAGGTCCGACAAATCGCACCGGGCCAAACCGGTAAA |
| | | ATTGCCGACTACAACTACAAGCTGCCTGACGACTTCACCGGCTGCGTAATCGCCTGGAACAGCAATAACCTGGATAG |
| | | CAAAGTGGGCGGAAACTACAACTACCTGTACCGGCTCTTTAGAAAGTCCAACCTGAAACCATTCGAGCGCGATATCTC |
| | | GACCGAAATCTACCAGGCGGGCAGCACCCCCTGTAATGGTGTAGAAGGGTTCAATTGTTACTTTCCACTCCAGAGTTA |
| | | TGGGTTCCAGCCGACCAATGGCGTCGGTTATCAACCATATCGCGTTGTGGTGTTGTCCTTTGAGCTGCTACACGCCC |
| | | CAGCTACAGTGTGCGGGCCAAAGAAAAGCACAAATCTTGTCAAGAACAAATGCGTTAACTTTAATTTTAATGGACTCAC |
| | | AGGTACAGGAGTCCTGACCGAATCTAATAAGAAGTTCCTGCCCTTTCAACAGTTCGGACGAGACATTGCCGACACCAC |
| | | CGATGCCGTTCGGGACCCACAGACCTTAGAAATTCTGGATATTACTCCATGTAGTTTTGGGGGAGTGTCTGTCATCAC |
| | | CCCTGGCACTAATACATCTAACCAGGTTGCAGTCCTCTACCAGGATGTGAACTGTACCGAAGTGCCGGTCGCTATTCA |
| | | CGCAGACCAGCTCACTCCTACCTGGCGGGTGTACTCCACAGGCTCTAACGTGTTTCAGACACGTGCAGGGTGCCTAA |
| | | TCGGCGCAGAGCATGTAAATAACTCCTATGAGTGTGATATCCCCATCGGAGCCGGGATCTGCGCTTCCTACCAGACA |
| | | CAAACGAATAGTCCCGGATCTGCCTCAAGCGTGGCATCTCAATCCATTATAGCATATACGATGTCCCTTGGAGCTGAA |
| | | AACAGCGTTGCGTATTCAAACAATAGTATCGCTATTCCAACCAATTTTACAATTAGCGTGACCACAGAAATACTCCCTG |
| | | TGAGCATGACCAAGACCAGTGTAGACTGTACTATGTACATCTGCGGCGACAGTACTGAGTGTAGCAATCTGCTGCTAC |
| | | AGTATGGGTCCTTCTGTACTCAGCTTAATCGGGCTCTCACCGGAATCGCTGTAGAGCAAGATAAAAACACACAAGAAG |
| | | TGTTTGCTCAAGTGAAGCAGATCTATAAGACACCTCCCATCAAGGATTTCGGTGGGTTCAACTTTAGCCAGATTCTGC |
| | | CCGATCCGTCTAAACCGTCCAAGCGAAGTTTCATCGAAGACCTGCTTTTCAATAAGGTCACGCTGGCAGATGCTGGAT |
| | | TTATCAAACAGTACGGCGACTGTCTGGGCGATATCGCCGCAAGAGACTTGATATGCGCCCAAAAGTTTAATGGGTTAA |
| | | CCGTCCTTCCACCGCTCCTGACAGACGAGATGATCGCCCAGTATACAAGTGCCTTATTAGCTGGGACCATTACTAGTG |
| | | GATGGACATTTGGCGCCGGGGCTGCTCTACAGATACCCTTCGCCATGCAGATGGCTTACCGCTTCAACGGAATCGGA |
| | | GTTACCCAGAACGTACTGTACGAAAATCAGAAACTCATAGCTAATCAATTTAACTCTGCCATCGGGAAGATTCAGGATT |
| | | CCCTGTCGTCTACAGCGTCCGCCTTGGGGAAACTGCAAGATGTAGTGAACCAGAACGCCCAGGCCTTAAATACTCTG |
| | | GTCAAGCAGTTATCTTCAAATTTCGGAGCAATTAGCTCTGTGTTGAACGATATTCTTTCCAGGCTGGACCCTCCAGAAG |
| | | CCGAAGTGCAAATAGACCGGCTCATCACGGGGCGCTTGCAAAGCCTGCAAACCTATGTCACCCAGCAACTGATTCGA |
| | | GCAGCCGAGATCCGGGCCAGTGCTAATCTGGCCGCCACAAAAATGAGCGAGTGCGTCCTCGGGCAGAGCAAACGCG |
| | | TAGACTTCTGCGGTAAAGGCTATCACCTGATGAGCTTCCCTCAGAGCGCACCCCACGGGGTGGTCTTCCTCCACGTT |
| | | ACCTACGTCCCTGCGCAGGAGAAGAACTTCACTACGGCCCCTGCAATTTGCCACGATGGCAAGGCCCACTTTCCCAG |
| | | GGAGGGGGTCTTCGTTTCCAACGGGACTCATTGGTTCGTGACTCAGAGAAATTTTTATGAACCTCAGATCATTACCAC |
| | | TGATAATACATTCGTGTCTGGCAACTGTGATGTGGTTATTGGGATAGTTAATAATACGGTATACGACCCACTCCAGCCC |
| | | GAGCTGGACTCCTTCAAAGAGGAGCTGGACAAGTACTTTAAAAATCACACCTCACCTGATGTGGACCTAGGTGACATA |
| | | TCTGGCATAAATGCTAGCGTGGTTAACATTCAGAAGGAAATCGACAGACTCAACGAGGTGGCCAAAAATCTGAACGAG |
| | | AGTCTGATCGACCTGCAGGAGTTGGGAAAATATGAACAGTACATCAAATGGCCATGGTACATCTGGCTGGGCTTCATA |
| | | GCAGGCCTGATCGCCATCGTCATGGTGACTATTATGCTGTGCTGCATGACATCCTGTTGTAGCTGTTTGAAGGGGTGT |
| | | TGCTCCTGCGGCTCATGCTGCAAATTCGACGAGGACGATTCAGAACCTGTGCTGAAGGGAGTGAAGCTGCATTACAC |
| | | ATAA |
| 30 | ORF41 | ATGTTCGTTTTCCTTGTTTTACTGCCCCTCGTGTCTTCACAGTGTAGGGTTCAGCCTACTGAGTCCATCGTGAGATTCC |
| | | CAAACATCACAAATCTTTGTCCCTTCGGTGAGGTATTCAATGCGACACGATTTGCCTCAGTGTACGCGTGGAATCGGA |
| | | AGAGGATCTCCAATTGCGTGGCCGACTACTCCGTCTTATACAACTCAGCTAGCTTTTCAACATTCAAGTGCTATGGCG |
| | | TGAGCCCTACCAAGCTCAATGACCTGTGCTTCACTAATGTGTATGCCGACTCTTTTGTCATTCGCGGCGACGAGGTCC |
| | | GACAAATCGCACCGGGCCAAACCGGTAAAATTGCCGACTACAACTACAAGCTGCCTGACGACTTCACCGGCTGCGTA |
| | | ATCGCCTGGAACAGCAATAACCTGGATAGCAAAGTGGGCGGAAACTACAACTACCTGTACCGGCTCTTTAGAAAGTC |
| | | CAACCTGAAACCATTCGAGCGCGATATCTCGACCGAAATCTACCAGGGGGCAGCACCCCCTGTAATGGTGTAGAAG |
| | | GGTTCAATTGTTACTTTCCACTCCAGAGTTATGGGTTCCAGCCGACCAATGGCGTCGGTTATCAACCATATCGCGTTG |
| | | TGGTGTTGTCCTTTGAGCTGCTACACGCCCCAGCTACAGTGTGCGGGCCAAAGAAAAGCACAAATCTTGTCAAGAAC |
| | | AAATGCGTTAACTTTTAA |
| 31 | EMCV IRES | ACGTTACTGGCCGAAGCCGCTTGGAATAAGGCCGGTGTGCGTTTGTCTATATGTTATTTTCCACCATATTGCCGTCTTT |
| | | TGGCAATGTGAGGGCCCGGAAACCTGGCCCTGTCTTCTTGACGAGCATTCCTAGGGGTCTTTCCCCTCTCGCCAAAG |
| | | GAATGCAAGGTCTGTTGAATGTCGTGAAGGAAGCAGTTCCTCTGGAAGCTTCTTGAAGACAAACAACGTCTGTAGCGA |
| | | CCCTTTGCAGGCAGCGGAACCCCCCACCTGGCGACAGGTGCCTCTGCGGCCAAAAGCCACGTGTATAAGATACACC |
| | | TGCAAAGGCGGCACAACCCCAGTGCCACGTTGTGAGTTGGATAGTTGTGGAAAGAGTCAAATGGCTCTCCTCAAGCG |
| | | TATTCAACAAGGGGCTGAAGGATGCCCAGAAGGTACCCCATTGTATGGGATCTGATCTGGGGCCTCGGTGCACATGC |
| | | TTTACATGTGTTTAGTCGAGGTTAAAAAACGTCTAGGCCCCCCGAACCACGGGGACGTGGTTTTCCTTTGAAAAACAC |
| | | GATGATAATA |
| 32 | 5′ UTR | ACTCTTCTGGTCCCCACAGACTCAGAGAGAACCCACC |
| | Globin | |
| 33 | 3′ UTR | GCTGGAGCCTCGGTAGCCGTTCCTCCTGCCCGCTGGGCCTCCCAACGGGCCCTCCTCCCCTCCTTGCACCGGCCCT |
| | Globin | TCCTGGTCTTTGAATAAAGTCTGAGTGGGCAGCA |
| 45 | CVB3 | TTAAAACAGCCTGTGGGTTGATCCCACCCACAGGCCCATTGGGCGCTAGCACTCTGGTATCACGGTACCTTTGTGCG |
| | | CCTGTTTTATACCCCCTCCCCCAACTGTAACTTAGAAGTAACACACACCGATCAACAGTCAGCGTGGCACACCAGCCA |
| | | CGTTTTGATCAAGCACTTCTGTTACCCCGGACTGAGTATCAATAGACTGCTCACGCGGTTGAAGGAGAAAGCGTTCGT |
| | | TATCCGGCCAACTACTTCGAAAAACCTAGTAACACCGTGGAAGTTGCAGAGTGTTTCGCTCAGCACTACCCCAGTGTA |
| | | GATCAGGTCGATGAGTCACCGCATTCCCCACGGGCGACCGTGGCGGTGGCTGCGTTGGCGGCCTGCCCATGGGGA |
| | | AACCCATGGGACGCTCTAATACAGACATGGTGCGAAGAGTCTATTGAGCTAGTTGGTAGTCCTCCGGCCCCTGAATG |
| | | CGGCTAATCCTAACTGCGGAGCACACACCCTCAAGCCAGAGGGCAGTGTGTCGTAACGGGCAACTCTGCAGCGGAA |
| | | CCGACTACTTTGGGTGTCCGTGTTTCATTTTATTCCTATACTGGCTGCTTATGGTGACAATTGAGAGATCGTTACCATA |
| | | TAGCTATTGGATTGGCCATCCGGTGACTAATAGAGCTATTATATATCCCTTTGTTGGGTTTATACCACTTAGCTTGAAA |
| | | GAGGTTAAAACATTACAATTCATTGTTAAGTTGAATACAGCAAA |
| 46 | WT EMCV | CGCGGATCCTAATACGACTCACTATAGGGAATAGCCGAAAAACAAAAAACAAAAAAAACAAAAAAAAAACCAAAAAAAC |
| | GLuc IS | AAAACACAACGTTACTGGCCGAAGCCGCTTGGAATAAGGCCGGTGTGCGTTTGTCTATATGTTATTTTCCACCATATT |
| | | GCCGTCTTTTGGCAATGTGAGGGCCCGGAAACCTGGCCCTGTCTTCTTGACGAGCATTCCTAGGGGTCTTTCCCCTC |
| | | TCGCCAAAGGAATGCAAGGTCTGTTGAATGTCGTGAAGGAAGCAGTTCCTCTGGAAGCTTCTTGAAGACAAACAACGT |
| | | CTGTAGCGACCCTTTGCAGGCAGCGGAACCCCCCACCTGGCGACAGGTGCCTCTGCGGCCAAAAGCCACGTGTATA |
| | | AGATACACCTGCAAAGGCGGCACAACCCCAGTGCCACGTTGTGAGTTGGATAGTTGTGGAAAGAGTCAAATGGCTCT |
| | | CCTCAAGCGTATTCAACAAGGGGCTGAAGGATGCCCAGAAGGTACCCCATTGTATGGGATCTGATCTGGGGCCTCGG |
| | | TGCACATGCTTTACATGTGTTTAGTCGAGGTTAAAAAACGTCTAGGCCCCCCGAACCACGGGGACGTGGTTTTCCTTT |
| | | GAAAAACACGATGATAATAGCCACCATGGGAGTCAAAGTTCTGTTTGCCCTGATCTGCATCGCTGTGGCCGAGGCCA |
| | | AGCCCACCGAGAACAACGAAGACTTCAACATCGTGGCCGTGGCCAGCAACTTCGCGACCACGGATCTCGATGCTGA |
| | | CCGCGGGAAGTTGCCCGGCAAGAAGCTGCCGCTGGAGGTGCTCAAAGAGATGGAAGCCAATGCCCGGAAAGCTGG |
| | | CTGCACCAGGGGCTGTCTGATCTGCCTGTCCCACATCAAGTGCACGCCCAAGATGAAGAAGTTCATCCCAGGACGCT |
| | | GCCACACCTACGAAGGCGACAAAGAGTCCGCACAGGGGGGCATAGGCGAGGCGATCGTCGACATTCCTGAGATTCC |
| | | TGGGTTCAAGGACTTGGAGCCCATGGAGCAGTTCATCGCACAGGTCGATCTGTGTGTGGACTGCACAACTGGCTGCC |
| | | TCAAAGGGCTTGCCAACGTGCAGTGTTCTGACCTGCTCAAGAAGTGGCTGCCGCAACGCTGTGCGACCTTTGCCAGC |
| | | AAGATCCAGGGCCAGGTGGACAAGATCAAGGGGGCCGGTGGTGACTAAAAAAAACAAAAAACAAAACGGCTATT |
| 47 | Splint | GTTTTTCGGCTATTCCCAATAGCCGTTTTG |
| 48 | ORF44 | ATGTACCGGATGCAGTTGTTGTCCTGTATAGCTCTTTCCCTTGCATTGGTCACTAATTCTagagtccaaccaacagaatctattgtta |
| | | gatttcctaatattacaaacttgtgcccttttggtgaagtttttaacgccaccagatttgcatccgtgtatgcttggaacaggaagagaatcagcaactgtgttgctgattattctgtcct |
| | | atataattccgcatcattttccacttttaagtgttatggagtgtctcctactaaattaaatgatctctgctttactaatgtctatgcagattcatttgtaattagaggtgatgaagtcagaca |
| | | aatcgctccagggcaaactggaaagattgctgattataattataaattaccagatgattttacaggctgcgttatagcttggaattctaacaatcttgattctaaggttggtggtaatt |
| | | ataattacctgtatagattgtttaggaagtctaatctcaaaccttttgagagagatatttcaactgaaatctatcaggccggtagcacaccttgtaatggtgttgaaggttttaattgtt |
| | | actttcctttacaatcatatggtttccaacccactaatggtgttggttaccaaccatacagagtagtagtactttcttttgaacttctacatgcaccagcaactgtttgtggacctaaaa |
| | | agtctactaatttggttaaaaacaaatgtgtcaatttcTAA |
| 49 | ORF45 | ATGGGAGTCAAAGTTCTGTTTGCCCTGATCTGCATCGCTGTGGCCGAGGCCAAGagagtccaaccaacagaatctattgttagatttcc |
| | | taatattacaaacttgtgcccttttggtgaagtttttaacgccaccagatttgcatccgtgtatgcttggaacaggaagagaatcagcaactgtgttgctgattattctgtcctatataa |
| | | ttccgcatcattttccacttttaagtgttatggagtgtctcctactaaattaaatgatctctgctttactaatgtctatgcagattcatttgtaattagaggtgatgaagtcagacaaatcg |
| | | ctccagggcaaactggaaagattgctgattataattataaattaccagatgattttacaggctgcgttatagcttggaattctaacaatcttgattctaaggttggtggtaattataatt |
| | | acctgtatagattgtttaggaagtctaatctcaaaccttttgagagagatatttcaactgaaatctatcaggccggtagcacaccttgtaatggtgttgaaggttttaattgttactttcc |
| | | tttacaatcatatggtttccaacccactaatggtgttggttaccaaccatacagagtagtagtactttcttttgaacttctacatgcaccagcaactgtttgtggacctaaaaagtcta |
| | | ctaatttggttaaaaacaaatgtgtcaatttcTAA |
| 50 | IL2 | ATGTACCGGATGCAGTTGTTGTCCTGTATAGCTCTTTCCCTTGCATTGGTCACTAATTCT |
| | secretion | |
| | signal | |
| 51 | Gluc | ATGGGAGTCAAAGTTCTGTTTGCCCTGATCTGCATCGCTGTGGCCGAGGCCAAG |
| | secretion | |
| | signal | |
| 52 | Synthetic | D(V/I)ExNPGP |
| | Construct | |
| 53 | Severe | MFVFLVLLPLVSSQCVNLTTRTQLPPAYTNSFTRGVYYPDKVFRSSVLHSTQDLFLPFFSNVTWFHAIHVSGTNGTKRFDN |
| | acute | PVLPFNDGVYFASTEKSNIIRGWIFGTTLDSKTQSLLIVNNATNVVIKVCEFQFCNDPFLGVYYHKNNKSWMESEFRVYSSA |
| | respiratory | NNCTFEYVSQPFLMDLEGKQGNFKNLREFVFKNIDGYFKIYSKHTPINLVRDLPQGFSALEPLVDLPIGINITRFQTLLALHRS |
| | syndrome | YLTPGDSSSGWTAGAAAYYVGYLQPRTFLLKYNENGTITDAVDCALDPLSETKCTLKSFTVEKGIYQTSNFRVQPTESIVRF |
| | coronavirus | PNITNLCPFGEVFNATRFASVYAWNRKRISNCVADYSVLYNSASFSTFKCYGVSPTKLNDLCFTNVYADSFVIRGDEVRQIA |
| | 2 | PGQTGKIADYNYKLPDDFTGCVIAWNSNNLDSKVGGNYNYLYRLFRKSNLKPFERDISTEIYQAGSTPCNGVEGFNCYFPL |
| | | QSYGFQPTNGVGYQPYRVVVLSFELLHAPATVCGPKKSTNLVKNKCVNFNFNGLTGTGVLTESNKKFLPFQQFGRDIADTT |
| | | DAVRDPQTLEILDITPCSFGGVSVITPGTNTSNQVAVLYQDVNCTEVPVAIHADQLTPTWRVYSTGSNVFQTRAGCLIGAEH |
| | | VNNSYECDIPIGAGICASYQTQTNSPRRARSVASQSIIAYTMSLGAENSVAYSNNSIAIPTNFTISVTTEILPVSMTKTSVDCT |
| | | MYICGDSTECSNLLLQYGSFCTQLNRALTGIAVEQDKNTQEVFAQVKQIYKTPPIKDFGGFNFSQILPDPSKPSKRSFIEDLL |
| | | FNKVTLADAGFIKQYGDCLGDIAARDLICAQKFNGLTVLPPLLTDEMIAQYTSALLAGTITSGWTFGAGAALQIPFAMQMAYR |
| | | FNGIGVTQNVLYENQKLIANQFNSAIGKIQDSLSSTASALGKLQDVVNQNAQALNTLVKQLSSNFGAISSVLNDILSRLDPPE |
| | | AEVQIDRLITGRLQSLQTYVTQQLIRAAEIRASANLAATKMSECVLGQSKRVDFCGKGYHLMSFPQSAPHGVVFLHVTYVPA |
| | | QEKNFTTAPAICHDGKAHFPREGVFVSNGTHWFVTQRNFYEPQIITTDNTFVSGNCDVVIGIVNNTVYDPLQPELDSFKEEL |
| | | DKYFKNHTSPDVDLGDISGINASVVNIQKEIDRLNEVAKNLNESLIDLQELGKYEQYIKWPWYIWLGFIAGLIAIVMVTIMLCC |
| | | MTSCCSCLKGCCSCGSCCKFDEDDSEPVLKGVKLHYT |
| 54 | Severe | atgtttgtttttcttgttttattgccactagtctctagtcagtgtgttaatcttacaaccagaactcaattaccccctgcatacactaattctttcacacgtggtgtttattaccctgacaa |
| | acute | agttttcagatcctcagttttacattcaactcaggatttgttcttacctttcttttccaatgttacttggttccatgctatacatgtctctgggaccaatggtactaagaggtttgataacc |
| | respiratory | ctgtcctaccatttaatgatggtgtttattttgcttccactgagaagtctaacataataagaggctggatctttggtactactttagattcgaagacccagtccctacttattgttaataac |
| | syndrome | gctactaatgttgttattaaagtctgtgaatttcaattttgtaatgatccatttttgggtgtttattaccacaaaaacaacaaaagttggatggaaagtgagttcagagtttattctagtgc |
| | coronavirus | gaataattgcacttttgaatatgtctctcagccttttcttatggaccttgaaggaaaacagggtaatttcaaaaatcttagggaatttgtgtttaagaatattgatggttattttaaaatat |
| | 2 | attctaagcacacgcctattaatttagtgcgtgatctccctcagggtttttcggctttagaaccattggtagatttgccaataggtattaacatcactaggtttcaaactttacttgcttta |
| | | catagaagttatttgactcctggtgattcttcttcaggttggacagctggtgctgcagcttattatgtgggttatcttcaacctaggacttttctattaaaatataatgaaaatggaaccat |
| | | tacagatgctgtagactgtgcacttgaccctctctcagaaacaaagtgtacgttgaaatccttcactgtagaaaaaggaatctatcaaacttctaactttagagtccaaccaacaga |
| | | atctattgttagatttcctaatattacaaacttgtgcccttttggtgaagtttttaacgccaccagatttgcatccgtgtatgcttggaacaggaagagaatcagcaactgtgttgctga |
| | | ttattctgtcctatataattccgcatcattttccacttttaagtgttatggagtgtctcctactaaattaaatgatctctgctttactaatgtctatgcagattcatttgtaattagaggtg |
| | | atgaagtcagacaaatcgctccagggcaaactggaaagattgctgattataattataaattaccagatgattttacaggctgcgttatagcttggaattctaacaatcttgattctaaggtt |
| | | ggtggtaattataattacctgtatagattgtttaggaagtctaatctcaaaccttttgagagagatatttcaactgaaatctatcaggccggtagcacaccttgtaatggtgttgaag |
| | | gttttaattgttactttcctttacaatcatatggtttccaacccactaatggtgttggttaccaaccatacagagtagtagtactttcttttgaacttctacatgcaccagcaactgtttgt |
| | | ggacctaaaaagtctactaatttggttaaaaacaaatgtgtcaatttcaacttcaatggtttaacaggcacaggtgttcttactgagtctaacaaaaagtttctgcctttccaacaattt |
| | | ggcagagacattgctgacactactgatgctgtccgtgatccacagacacttgagattcttgacattacaccatgttcttttggtggtgtcagtgttataacaccaggaacaaatact |
| | | tctaaccaggttgctgttctttatcaggatgttaactgcacagaagtccctgttgctattcatgcagatcaacttactcctacttggcgtgtttattctacaggttctaatgtttttcaaac |
| | | acgtgcaggctgtttaataggggctgaacatgtcaacaactcatatgagtgtgacatacccattggtgcaggtatatgcgctagttatcagactcagactaattctcctcggggg |
| | | cacgtagtgtagctagtcaatccatcattgcctacactatgtcacttggtgcagaaaattcagttgcttactctaataactctattgccatacccacaaattttactattagtgttacca |
| | | cagaaattctaccagtgtctatgaccaagacatcagtagattgtacaatgtacatttgtggtgattcaactgaatgcagcaatcttttgttgcaatatggcagtttttgtacacaatta |
| | | aaccgtgctttaactgggatagctgttgaacaagacaaaaacacccaagaagtttttgcacaagtcaaacaaatttacaaaacaccaccaattaaagattttggtggttttaatt |
| | | tttcacaaatattaccagatccatcaaaaccaagcaagaggtcatttattgaagatctacttttcaacaaagtgacacttgcagatgctggcttcatcaaacaatatggtgattgc |
| | | cttggtgatattgctgctagggacctcatttgtgcacaaaagtttaacggccttactgttttgccacctttgctcacagatgaaatgattgctcaatacacttctgcactgttagcgggt |
| | | acaatcacttctggttggacctttggtgcaggtgctgcattacaaataccatttgctatgcaaatggcttataggtttaatggtattggagttacacagaatgttctctatgagaacca |
| | | aaaattgattgccaaccaatttaatagtgccattggcaaaattcaagactcactttcttccacagcaagtgcacttggaaaacttcaagatgtggtcaaccaaaatgcacaagc |
| | | tttaaacacgcttgttaaacaacttagctccaattttggtgcaatttcaagtgttttaaatgatatcctttcacgtcttgaccctcccgaggctgaagtgcaaattgataggttgatcac |
| | | aggcagacttcaaagtttgcagacatatgtgactcaacaattaattagagctgcagaaatcagagcttctgctaatcttgctgctactaaaatgtcagagtgtgtacttggacaat |
| | | caaaaagagttgatttttgtggaaagggctatcatcttatgtccttccctcagtcagcacctcatggtgtagtcttcttgcatgtgacttatgtccctgcacaagaaaagaacttcac |
| | | aactgctcctgccatttgtcatgatggaaaagcacactttcctcgtgaaggtgtctttgtttcaaatggcacacactggtttgtaacacaaaggaatttttatgaaccacaaatcatt |
| | | actacagacaacacatttgtgtctggtaactgtgatgttgtaataggaattgtcaacaacacagtttatgatcctttgcaacctgaattagactcattcaaggaggagttagataaa |
| | | tattttaagaatcatacatcaccagatgttgatttaggtgacatctctggcattaatgcttcagttgtaaacattcaaaaagaaattgaccgcctcaatgaggttgccaagaatttaa |
| | | atgaatctctcatcgatctccaagaacttggaaagtatgagcagtatataaaatggccatggtacatttggctaggttttatagctggcttgattgccatagtaatggtgacaattat |
| | | gctttgctgtatgaccagttgctgtagttgtctcaagggctgttgttcttgtggatcctgctgcaaatttgatgaagacgactctgagccagtgctcaaaggagtcaaattacattac |
| | | aca |
| 55 | Synthetic | MGVKVLFALICIAVAEAKRVQPTESIVRFPNITNLCPFGEVFNATRFASVYAWNRKRISNCVADYSVLYNSASFSTFKCYGVS |
| | Construct | PTKLNDLCFTNVYADSFVIRGDEVRQIAPGQTGKIADYNYKLPDDFTGCVIAWNSNNLDSKVGGNYNYLYRLFRKSNLKPFE |
| | | RDISTEIYQAGSTPCNGVEGFNCYFPLQSYGFQPTNGVGYQPYRVVVLSFELLHAPATVCGPKKSTNLVKNKCVNF |
| 56 | Synthetic | atgggagtcaaagttctgtttgccctgatctgcattgctgtggccgaggccaagagagtccaaccaacagaatctattgttagatttcctaatattacaaacttgtgcccttttggtg |
| | Construct | aagtttttaacgccaccagatttgcatccgtgtatgcttggaacaggaagagaatcagcaactgtgttgctgattattctgtcctatataattccgcatcattttccacttttaagtgttat |
| | | ggagtgtctcctactaaattaaatgatctctgctttactaatgtctatgcagattcatttgtaattagaggtgatgaagtcagacaaatcgctccagggcaaactggaaagattgct |
| | | gattataattataaattaccagatgattttacaggctgcgttatagcttggaattctaacaatcttgattctaaggttggtggtaattataattacctgtatagattgtttaggaagtctaa |
| | | tctcaaaccttttgagagagatatttcaactgaaatctatcaggccggtagcacaccttgtaatggtgttgaaggttttaattgttactttcctttacaatcatatggtttccaacccact |
| | | aatggtgttggttaccaaccatacagagtagtagtactttcttttgaacttctacatgcaccagcaactgtttgtggacctaaaaagtctactaatttggttaaaaacaaatgtgtca |
| | | atttc |
| 57 | Synthetic | MGVKVLFALICIAVAEAKPTENNEDFNIVAVASNFATTDLDADRGKLPGKKLPLEVLKEMEANARKAGCTRGCLICLSHIKCT |
| | Construct | PKMKKFIPGRCHTYEGDKESAQGGIGEAIVDIPEIPGFKDLEPMEQFIAQVDLCVDCTTGCLKGLANVQCSDLLKKWLPQRC |
| | | ATFASKIQGQVDKIKGAGGD |
| 58 | Synthetic | atgggagtcaaagttctgtttgccctgatctgcatcgctgtggccgaggccaagcccaccgagaacaacgaagacttcaacatcgtggccgtggccagcaacttcgcgacc |
| | Construct | acggatctcgatgctgaccgcgggaagttgcccggcaagaagctgccgctggaggtgctcaaagagatggaagccaatgcccggaaagctggctgcaccaggggctgt |
| | | ctgatctgcctgtcccacatcaagtgcacgcccaagatgaagaagttcatcccaggacgctgccacacctacgaaggcgacaaagagtccgcacagggcggcataggc |
| | | gaggcgatcgtcgacattcctgagattcctgggttcaaggacttggagcccatggagcagttcatcgcacaggtcgatctgtgtgtggactgcacaactggctgcctcaaagg |
| | | gcttgccaacgtgcagtgttctgacctgctcaagaagtggctgccgcaacgctgtgcgacctttgccagcaagatccagggccaggtggacaagatcaagggggccggtg |
| | | gtgactaa |
| 59 | Influenza | MKAILVVLLYTFATANADTLCIGYHANNSTDTVDTVLEKNVTVTHSVNLLEDKHNGKLCKLRGVAPLHLGKCNIAGWILGNPE |
| | A virus | CESLSTASSWSYIVETPSSDNGTCYPGDFIDYEELREQLSSVSSFERFEIFPKTSSWPNHDSNKGVTAACPHAGAKSFYKN |
| | | LIWLVKKGNSYPKLSKSYINDKGKEVLVLWGIHHPSTSADQQSLYQNADAYVFVGSSRYSKKFKPEIAIRPKVRDQEGRMN |
| | | YYWTLVEPGDKITFEATGNLVVPRYAFAMERNAGSGIIISDTPVHDCNTTCQTPKGAINTSLPFQNIHPITIGKCPKYVKSTKL |
| | | RLATGLRNIPSIQSRGLFGAIAGFIEGGWTGMVDGWYGYHHQNEQGSGYAADLKSTQNAIDEITNKVNSVIEKMNTQFTAV |
| | | GKEFNHLEKRIENLNKKVDDGFLDIWTYNAELLVLLENERTLDYHDSNVKNLYEKVRSQLKNNAKEIGNGCFEFYHKCDNT |
| | | CMESVKNGTYDYPKYSEEAKLNREEIDGVKLESTRIYQILAIYSTVASSLVLVVSLGAISFWMCSNGSLQCRICI |
| 60 | Influenza | atgaaagcaatactagtagttcttctatatacatttgcaaccgctaacgctgatacattgtgtataggatatcacgcgaacaactccaccgatacagtagatacagtactagaga |
| | A virus | agaacgtaacagtaacacattctgttaatcttctagaagacaagcataacggcaaactgtgcaaactaagaggtgtagccccattgcatctaggaaagtgtaatatagctgg |
| | | ctggattttgggaaatccagagtgtgaatcattaagtacagcaagctcctggtcctatatagtggaaacacctagtagtgataacggaacgtgttacccaggagattttatagatt |
| | | acgaggagctaagagagcagctgtcgtcagtatcatcatttgaaaggtttgaaattttcccgaaaacatcctcctggcccaatcacgatagtaacaaaggagtaacagcagc |
| | | ctgtcctcacgctggagcaaaaagcttctataaaaatttaatctggctagtgaagaagggaaattcatatccaaagctaagtaaaagttatataaacgataagggcaaggaa |
| | | gtactcgtactgtggggcattcatcatccatctactagtgctgatcaacaaagtttatatcaaaacgcagacgcatacgtttttgtggggtcaagtagatatagcaagaaatttaa |
| | | accagaaatagcaataagacctaaagtaagggatcaagaaggcagaatgaactattattggacactagtagaaccgggagataaaataacttttgaagcaacaggaaat |
| | | ctagtggttcccaggtacgcatttgcaatggaaagaaacgctggatcaggcatcattatatctgatacaccagtccacgattgtaatacaacttgtcaaacacctaaaggagct |
| | | ataaacaccagcttaccatttcaaaatattcatcctatcacaattggaaagtgtccaaaatacgtaaaaagtacaaaattgagattggccacaggattacgaaatattccatca |
| | | attcaatctagaggactttttggtgcaattgcaggtttcatagaaggaggctggactgggatggtagacggctggtacggttatcatcatcaaaacgaacagggaagtggata |
| | | cgcagctgatcttaaaagtacacaaaacgcaattgacgagattactaataaagtaaattctgtaattgaaaaaatgaatactcagtttacagcagtagggaaagagtttaacc |
| | | acctggaaaaaagaatagaaaatttaaataaaaaagtagacgacggatttcttgacatttggacttataacgccgaactattggtattactagaaaacgaaagaactctagat |
| | | tatcacgattcaaacgtaaaaaatttatacgaaaaagtaagaagccaacttaaaaataacgcaaaagaaataggaaacggctgttttgaattttatcacaagtgtgataatac |
| | | ctgcatggaaagtgttaaaaacgggacatacgattatccaaaatactcagaagaagcaaaattaaatagagaagaaatagacggcgtaaaattagaatcaacaaggata |
| | | tatcaaatattagcaatatattcaactgtcgcttcttcattggtactggtagtttctctaggtgcaatatcattttggatgtgctctaacggctccctacagtgtagaatttgtata |
| 61 | Hepatitis | ggccaccgacaagaggcggcagcccagaccacccgaaagaggagacgcggaccgccgcccaagcgagcagagcc |
| | D virus | |
| 62 | Hepatitis | ggcagaggcggcagcccagaccacccgaaagaggagacgcggaccgccgcccgagcc |
| | D virus | |
| 63 | P46 | MGVHECPAWLWLLLSLLSLPLGLPVLGARVQPTESIVRFPNITNLCPFGEVFNATRFASVYAWNRKRISNCVADYSVLYNSA |
| | | SFSTFKCYGVSPTKLNDLCFTNVYADSFVIRGDEVRQIAPGQTGKIADYNYKLPDDFTGCVIAWNSNNLDSKVGGNYNYLY |
| | | RLFRKSNLKPFERDISTEIYQAGSTPCNGVEGENCYFPLQSYGFQPTNGVGYQPYRVVVLSFELLHAPATVCGPKKSTNLV |
| | | KNKCVNF |
| 64 | P47 | MYRMQLLSCIALSLALVTNSRVQPTESIVRFPNITNLCPFGEVFNATRFASVYAWNRKRISNCVADYSVLYNSASFSTFKCY |
| | | GVSPTKLNDLCFTNVYADSFVIRGDEVRQIAPGQTGKIADYNYKLPDDFTGCVIAWNSNNLDSKVGGNYNYLYRLFRKSNL |
| | | KPFERDISTEIYQAGSTPCNGVEGFNCYFPLQSYGFQPTNGVGYQPYRVVVLSFELLHAPATVCGPKKSTNLVKNKCVNF |
| 65 | P48 | MGVKVLFALICIAVAEAKRVQPTESIVRFPNITNLCPFGEVFNATRFASVYAWNRKRISNCVADYSVLYNSASFSTFKCYGVS |
| | | PTKLNDLCFTNVYADSFVIRGDEVRQIAPGQTGKIADYNYKLPDDFTGCVIAWNSNNLDSKVGGNYNYLYRLFRKSNLKPFE |
| | | RDISTEIYQAGSTPCNGVEGFNCYFPLQSYGFQPTNGVGYQPYRVVVLSFELLHAPATVCGPKKSTNLVKNKCVNF |
| 66 | P49 | MGVHECPAWLWLLLSLLSLPLGLPVLGARVQPTESIVRFPNITNLCPFGEVFNATRFASVYAWNRKRISNCVADYSVLYNSA |
| | | SFSTFKCYGVSPTKLNDLCFTNVYADSFVIRGDEVRQIAPGQTGKIADYNYKLPDDFTGCVIAWNSNNLDSKVGGNYNYLY |
| | | RLFRKSNLKPFERDISTEIYQAGSTPCNGVEGFNCYFPLQSYGFQPTNGVGYQPYRVVVLSFELLHAPATVCGPKKSTNLV |
| | | KNKCVNF |
| 67 | P50 | MYRMQLLSCIALSLALVTNSRVQPTESIVRFPNITNLCPFGEVFNATRFASVYAWNRKRISNCVADYSVLYNSASFSTFKCY |
| | | GVSPTKLNDLCFTNVYADSFVIRGDEVRQIAPGQTGKIADYNYKLPDDFTGCVIAWNSNNLDSKVGGNYNYLYRLFRKSNL |
| | | KPFERDISTEIYQAGSTPCNGVEGENCYFPLQSYGFQPTNGVGYQPYRVVVLSFELLHAPATVCGPKKSTNLVKNKCVNF |
| 68 | P52 | MYRMQLLSCIALSLALVTNSVNLTTRTQLPPAYTNSFTRGVYYPDKVFRSSVLHSTQDLFLPFFSNVTWFHAIHVSGTNGTK |
| | | RFDNPVLPFNDGVYFASTEKSNIIRGWIFGTTLDSKTQSLLIVNNATNVVIKVCEFQFCNDPFLGVYYHKNNKSWMESEFRV |
| | | YSSANNCTFEYVSQPFLMDLEGKQGNFKNLREFVFKNIDGYFKIYSKHTPINLVRDLPQGFSALEPLVDLPIGINITRFQTLLA |
| | | LHRSYLTPGDSSSGWTAGAAAYYVGYLQPRTFLLKYNENGTITDAVDCALDPLSETKCTLKSFTVEKGIYQTSNFRVQPTE |
| | | SIVRFPNITNLCPFGEVFNATRFASVYAWNRKRISNCVADYSVLYNSASFSTFKCYGVSPTKLNDLCFTNVYADSFVIRGDE |
| | | VRQIAPGQTGKIADYNYKLPDDFTGCVIAWNSNNLDSKVGGNYNYLYRLFRKSNLKPFERDISTEIYQAGSTPCNGVEGEN |
| | | CYFPLQSYGFQPTNGVGYQPYRVVVLSFELLHAPATVCGPKKSTNLVKNKCVNFNFNGLTGTGVLTESNKKFLPFQQFGR |
| | | DIADTTDAVRDPQTLEILDITPCSFGGVSVITPGTNTSNQVAVLYQDVNCTEVPVAIHADQLTPTWRVYSTGSNVFQTRAGC |
| | | LIGAEHVNNSYECDIPIGAGICASYQTQTNSPGSASSVASQSIIAYTMSLGAENSVAYSNNSIAIPTNFTISVTTEILPVSMTKT |
| | | SVDCTMYICGDSTECSNLLLQYGSFCTQLNRALTGIAVEQDKNTQEVFAQVKQIYKTPPIKDFGGFNFSQILPDPSKPSKRS |
| | | FIEDLLFNKVTLADAGFIKQYGDCLGDIAARDLICAQKFNGLTVLPPLLTDEMIAQYTSALLAGTITSGWTFGAGAALQIPFAM |
| | | QMAYRFNGIGVTQNVLYENQKLIANQFNSAIGKIQDSLSSTASALGKLQDVVNQNAQALNTLVKQLSSNFGAISSVLNDILSR |
| | | LDPPEAEVQIDRLITGRLQSLQTYVTQQLIRAAEIRASANLAATKMSECVLGQSKRVDFCGKGYHLMSFPQSAPHGVVFLHV |
| | | TYVPAQEKNFTTAPAICHDGKAHFPREGVFVSNGTHWFVTQRNFYEPQIITTDNTFVSGNCDVVIGIVNNTVYDPLQPELDS |
| | | FKEELDKYFKNHTSPDVDLGDISGINASVVNIQKEIDRLNEVAKNLNESLIDLQELGKYEQYIKWPWYIWLGFIAGLIAIVMVTI |
| | | MLCCMTSCCSCLKGCCSCGSCCKFDEDDSEPVLKGVKLHYT |
| 69 | P54 | MGVKVLFALICIAVAEAKVNLTTRTQLPPAYTNSFTRGVYYPDKVFRSSVLHSTQDLFLPFFSNVTWFHAIHVSGTNGTKRF |
| | | DNPVLPFNDGVYFASTEKSNIIRGWIFGTTLDSKTQSLLIVNNATNVVIKVCEFQFCNDPFLGVYYHKNNKSWMESEFRVYS |
| | | SANNCTFEYVSQPFLMDLEGKQGNFKNLREFVFKNIDGYFKIYSKHTPINLVRDLPQGFSALEPLVDLPIGINITRFQTLLALH |
| | | RSYLTPGDSSSGWTAGAAAYYVGYLQPRTFLLKYNENGTITDAVDCALDPLSETKCTLKSFTVEKGIYQTSNFRVQPTESIV |
| | | RFPNITNLCPFGEVFNATRFASVYAWNRKRISNCVADYSVLYNSASFSTFKCYGVSPTKLNDLCFTNVYADSFVIRGDEVR |
| | | QIAPGQTGKIADYNYKLPDDFTGCVIAWNSNNLDSKVGGNYNYLYRLFRKSNLKPFERDISTEIYQAGSTPCNGVEGENCY |
| | | FPLQSYGFQPTNGVGYQPYRVVVLSFELLHAPATVCGPKKSTNLVKNKCVNFNFNGLTGTGVLTESNKKFLPFQQFGRDIA |
| | | DTTDAVRDPQTLEILDITPCSFGGVSVITPGTNTSNQVAVLYQDVNCTEVPVAIHADQLTPTWRVYSTGSNVFQTRAGCLIG |
| | | AEHVNNSYECDIPIGAGICASYQTQTNSPGSASSVASQSIIAYTMSLGAENSVAYSNNSIAIPTNFTISVTTEILPVSMTKTSVD |
| | | CTMYICGDSTECSNLLLQYGSFCTQLNRALTGIAVEQDKNTQEVFAQVKQIYKTPPIKDFGGFNFSQILPDPSKPSKRSFIED |
| | | LLFNKVTLADAGFIKQYGDCLGDIAARDLICAQKFNGLTVLPPLLTDEMIAQYTSALLAGTITSGWTFGAGAALQIPFAMQMA |
| | | YRFNGIGVTQNVLYENQKLIANQFNSAIGKIQDSLSSTASALGKLQDVVNQNAQALNTLVKQLSSNFGAISSVLNDILSRLDP |
| | | PEAEVQIDRLITGRLQSLQTYVTQQLIRAAEIRASANLAATKMSECVLGQSKRVDFCGKGYHLMSFPQSAPHGVVFLHVTYV |
| | | PAQEKNFTTAPAICHDGKAHFPREGVFVSNGTHWFVTQRNFYEPQIITTDNTFVSGNCDVVIGIVNNTVYDPLQPELDSFKE |
| | | ELDKYFKNHTSPDVDLGDISGINASVVNIQKEIDRLNEVAKNLNESLIDLQELGKYEQYIKWPWYIWLGFIAGLIAIVMVTIMLC |
| | | CMTSCCSCLKGCCSCGSCCKFDEDDSEPVLKGVKLHYT |
| 70 | P56 | MFVFLVLLPLVSSQCRVQPTESIVRFPNITNLCPFGEVFNATRFASVYAWNRKRISNCVADYSVLYNSASFSTFKCYGVSPT |
| | | KLNDLCFTNVYADSFVIRGDEVRQIAPGQTGKIADYNYKLPDDFTGCVIAWNSNNLDSKVGGNYNYLYRLFRKSNLKPFER |
| | | DISTEIYQAGSTPCNGVEGENCYFPLQSYGFQPTNGVGYQPYRVVVLSFELLHAPATVCGPKKSTNLVKNKCVNFGYIPEA |
| | | PRDGQAYVRKDGEWVLLSTFL |
| 71 | P59 | MFVFLVLLPLVSSQCVNLTTRTQLPPAYTNSFTRGVYYPDKVFRSSVLHSTQDLFLPFFSNVTWFHAIHVSGTNGTKRFDN |
| | | PVLPFNDGVYFASTEKSNIIRGWIFGTTLDSKTQSLLIVNNATNVVIKVCEFQFCNDPFLGVYYHKNNKSWMESEFRVYSSA |
| | | NNCTFEYVSQPFLMDLEGKQGNFKNLREFVFKNIDGYFKIYSKHTPINLVRDLPQGFSALEPLVDLPIGINITRFQTLLALHRS |
| | | YLTPGDSSSGWTAGAAAYYVGYLQPRTFLLKYNENGTITDAVDCALDPLSETKCTLKSFTVEKGIYQTSNFRVQPTESIVRF |
| | | PNITNLCPFGEVFNATRFASVYAWNRKRISNCVADYSVLYNSASFSTFKCYGVSPTKLNDLCFTNVYADSFVIRGDEVRQIA |
| | | PGQTGKIADYNYKLPDDFTGCVIAWNSNNLDSKVGGNYNYLYRLFRKSNLKPFERDISTEIYQAGSTPCNGVEGFNCYFPL |
| | | QSYGFQPTNGVGYQPYRVVVLSFELLHAPATVCGPKKSTNLVKNKCVNFNFNGLTGTGVLTESNKKFLPFQQFGRDIADTT |
| | | DAVRDPQTLEILDITPCSFGGVSVITPGTNTSNQVAVLYQDVNCTEVPVAIHADQLTPTWRVYSTGSNVFQTRAGCLIGAEH |
| | | VNNSYECDIPIGAGICASYQTQTNSPRRARSVASQSIIAYTMSLGAENSVAYSNNSIAIPTNFTISVTTEILPVSMTKTSVDCT |
| | | MYICGDSTECSNLLLQYGSFCTQLNRALTGIAVEQDKNTQEVFAQVKQIYKTPPIKDFGGFNFSQILPDPSKPSKRSFIEDLL |
| | | FNKVTLADAGFIKQYGDCLGDIAARDLICAQKFNGLTVLPPLLTDEMIAQYTSALLAGTITSGWTFGAGAALQIPFAMQMAYR |
| | | FNGIGVTQNVLYENQKLIANQFNSAIGKIQDSLSSTASALGKLQDVVNQNAQALNTLVKQLSSNFGAISSVLNDILSRLDPPE |
| | | AEVQIDRLITGRLQSLQTYVTQQLIRAAEIRASANLAATKMSECVLGQSKRVDFCGKGYHLMSFPQSAPHGVVFLHVTYVPA |
| | | QEKNFTTAPAICHDGKAHFPREGVFVSNGTHWFVTQRNFYEPQIITTDNTFVSGNCDVVIGIVNNTVYDPLQPELDSFKEEL |
| | | DKYFKNHTSPDVDLGDISGINASVVNIQKEIDRLNEVAKNLNESLIDLQELGKYEQYIKWPWYIWLGFIAGLIAIVMVTIMLCC |
| | | MTSCCSCLKGCCSCGSCCKFDEDDSEPVLKGVKLHYT |
| 72 | P60 | MFVFLVLLPLVSSQCVNLTTRTQLPPAYTNSFTRGVYYPDKVFRSSVLHSTQDLFLPFFSNVTWFHAIHVSGTNGTKRFDN |
| | | PVLPFNDGVYFASTEKSNIIRGWIFGTTLDSKTQSLLIVNNATNVVIKVCEFQFCNDPFLGVYYHKNNKSWMESEFRVYSSA |
| | | NNCTFEYVSQPFLMDLEGKQGNFKNLREFVFKNIDGYFKIYSKHTPINLVRDLPQGFSALEPLVDLPIGINITRFQTLLALHRS |
| | | YLTPGDSSSGWTAGAAAYYVGYLQPRTFLLKYNENGTITDAVDCALDPLSETKCTLKSFTVEKGIYQTSNFRVQPTESIVRF |
| | | PNITNLCPFGEVFNATRFASVYAWNRKRISNCVADYSVLYNSASFSTFKCYGVSPTKLNDLCFTNVYADSFVIRGDEVRQIA |
| | | PGQTGKIADYNYKLPDDFTGCVIAWNSNNLDSKVGGNYNYLYRLFRKSNLKPFERDISTEIYQAGSTPCNGVEGFNCYFPL |
| | | QSYGFQPTNGVGYQPYRVVVLSFELLHAPATVCGPKKSTNLVKNKCVNFNFNGLTGTGVLTESNKKFLPFQQFGRDIADTT |
| | | DAVRDPQTLEILDITPCSFGGVSVITPGTNTSNQVAVLYQGVNCTEVPVAIHADQLTPTWRVYSTGSNVFQTRAGCLIGAEH |
| | | VNNSYECDIPIGAGICASYQTQTNSPRRARSVASQSIIAYTMSLGAENSVAYSNNSIAIPTNFTISVTTEILPVSMTKTSVDCT |
| | | MYICGDSTECSNLLLQYGSFCTQLNRALTGIAVEQDKNTQEVFAQVKQIYKTPPIKDFGGFNFSQILPDPSKPSKRSFIEDLL |
| | | FNKVTLADAGFIKQYGDCLGDIAARDLICAQKFNGLTVLPPLLTDEMIAQYTSALLAGTITSGWTFGAGAALQIPFAMQMAYR |
| | | FNGIGVTQNVLYENQKLIANQFNSAIGKIQDSLSSTASALGKLQDVVNQNAQALNTLVKQLSSNFGAISSVLNDILSRLDPPE |
| | | AEVQIDRLITGRLQSLQTYVTQQLIRAAEIRASANLAATKMSECVLGQSKRVDFCGKGYHLMSFPQSAPHGVVFLHVTYVPA |
| | | QEKNFTTAPAICHDGKAHFPREGVFVSNGTHWFVTQRNFYEPQIITTDNTFVSGNCDVVIGIVNNTVYDPLQPELDSFKEEL |
| | | DKYFKNHTSPDVDLGDISGINASVVNIQKEIDRLNEVAKNLNESLIDLQELGKYEQYIKWPWYIWLGFIAGLIAIVMVTIMLCC |
| | | MTSCCSCLKGCCSCGSCCKFDEDDSEPVLKGVKLHYT |
| 73 | P61 | MFVFLVLLPLVSSQCVNLTTRTQLPPAYTNSFTRGVYYPDKVFRSSVLHSTQDLFLPFFSNVTWFHAIHVSGTNGTKRFDN |
| | | PVLPFNDGVYFASTEKSNIIRGWIFGTTLDSKTQSLLIVNNATNVVIKVCEFQFCNDPFLGVYYHKNNKSWMESEFRVYSSA |
| | | NNCTFEYVSQPFLMDLEGKQGNFKNLREFVFKNIDGYFKIYSKHTPINLVRDLPQGFSALEPLVDLPIGINITRFQTLLALHRS |
| | | YLTPGDSSSGWTAGAAAYYVGYLQPRTFLLKYNENGTITDAVDCALDPLSETKCTLKSFTVEKGIYQTSNFRVQPTESIVRF |
| | | PNITNLCPFGEVFNATRFASVYAWNRKRISNCVADYSVLYNSASFSTFKCYGVSPTKLNDLCFTNVYADSFVIRGDEVRQIA |
| | | PGQTGKIADYNYKLPDDFTGCVIAWNSNNLDSKVGGNYNYLYRLFRKSNLKPFERDISTEIYQAGSTPCNGVEGFNCYFPL |
| | | QSYGFQPTNGVGYQPYRVVVLSFELLHAPATVCGPKKSTNLVKNKCVNFNFNGLTGTGVLTESNKKFLPFQQFGRDIADTT |
| | | DAVRDPQTLEILDITPCSFGGVSVITPGTNTSNQVAVLYQDVNCTEVPVAIHADQLTPTWRVYSTGSNVFQTRAGCLIGAEH |
| | | VNNSYECDIPIGAGICASYQTQTNSPRRARSVASQSIIAYTMSLGAENSVAYSNNSIAIPTNFTISVTTEILPVSMTKTSVDCT |
| | | MYICGDSTECSNLLLQYGSFCTQLNRALTGIAVEQDKNTQEVFAQVKQIYKTPPIKDFGGFNFSQILPDPSKPSKRSFIEDLL |
| | | FNKVTLADAGFIKQYGDCLGDIAARDLICAQKFNGLTVLPPLLTDEMIAQYTSALLAGTITSGWTFGAGAALQIPFAMQMAYR |
| | | FNGIGVTQNVLYENQKLIANQFNSAIGKIQDSLSSTASALGKLQDVVNQNAQALNTLVKQLSSNFGAISSVLNDILSRLDPPE |
| | | AEVQIDRLITGRLQSLQTYVTQQLIRAAEIRASANLAATKMSECVLGQSKRVDFCGKGYHLMSFPQSAPHGVVFLHVTYVPA |
| | | QEKNFTTAPAICHDGKAHFPREGVFVSNGTHWFVTQRNFYEPQIITTDNTFVSGNCDVVIGIVNNTVYDPLQPELDSFKEEL |
| | | DKYFKNHTSPDVDLGDISGINASVVNIQKEIDRLNEVAKNLNESLIDLQELGKYEQYIKWPWYIWLGFIAGLIAIVMVTIMLCC |
| | | MTSCCSCLKGCCSCGSCCKFDEDDSEPVLKGVKLHYT |
| 74 | P63 | MGVKVLFALICIAVAEAKRVQPTESIVRFPNITNLCPFGEVFNATRFASVYAWNRKRISNCVADYSVLYNSASFSTFKCYGVS |
| | | PTKLNDLCFTNVYADSFVIRGDEVRQIAPGQTGKIADYNYKLPDDFTGCVIAWNSNNLDSKVGGNYNYLYRLFRKSNLKPFE |
| | | RDISTEIYQAGSTPCNGVEGFNCYFPLQSYGFQPTNGVGYQPYRVVVLSFELLHAPATVCGPKKSTNLVKNKCVNF |
| 75 | P64 | MFVFLVLLPLVSSQCVNLTTRTQLPPAYTNSFTRGVYYPDKVFRSSVLHSTQDLFLPFFSNVTWFHAIHVSGTNGTKRFDN |
| | | PVLPFNDGVYFASTEKSNIIRGWIFGTTLDSKTQSLLIVNNATNVVIKVCEFQFCNDPFLGVYYHKNNKSWMESEFRVYSSA |
| | | NNCTFEYVSQPFLMDLEGKQGNFKNLREFVFKNIDGYFKIYSKHTPINLVRDLPQGFSALEPLVDLPIGINITRFQTLLALHRS |
| | | YLTPGDSSSGWTAGAAAYYVGYLQPRTFLLKYNENGTITDAVDCALDPLSETKCTLKSFTVEKGIYQTSNFRVQPTESIVRF |
| | | PNITNLCPFGEVFNATRFASVYAWNRKRISNCVADYSVLYNSASFSTFKCYGVSPTKLNDLCFTNVYADSFVIRGDEVRQIA |
| | | PGQTGKIADYNYKLPDDFTGCVIAWNSNNLDSKVGGNYNYLYRLFRKSNLKPFERDISTEIYQAGSTPCNGVEGFNCYFPL |
| | | QSYGFQPTNGVGYQPYRVVVLSFELLHAPATVCGPKKSTNLVKNKCVNFNFNGLTGTGVLTESNKKFLPFQQFGRDIADTT |
| | | DAVRDPQTLEILDITPCSFGGVSVITPGTNTSNQVAVLYQDVNCTEVPVAIHADQLTPTWRVYSTGSNVFQTRAGCLIGAEH |
| | | VNNSYECDIPIGAGICASYQTQTNSPRRARSVASQSIIAYTMSLGAENSVAYSNNSIAIPTNFTISVTTEILPVSMTKTSVDCT |
| | | MYICGDSTECSNLLLQYGSFCTQLNRALTGIAVEQDKNTQEVFAQVKQIYKTPPIKDFGGFNFSQILPDPSKPSKRSFIEDLL |
| | | FNKVTLADAGFIKQYGDCLGDIAARDLICAQKFNGLTVLPPLLTDEMIAQYTSALLAGTITSGWTFGAGAALQIPFAMQMAYR |
| | | FNGIGVTQNVLYENQKLIANQFNSAIGKIQDSLSSTASALGKLQDVVNQNAQALNTLVKQLSSNFGAISSVLNDILSRLDPPE |
| | | AEVQIDRLITGRLQSLQTYVTQQLIRAAEIRASANLAATKMSECVLGQSKRVDFCGKGYHLMSFPQSAPHGVVFLHVTYVPA |
| | | QEKNFTTAPAICHDGKAHFPREGVFVSNGTHWFVTQRNFYEPQIITTDNTFVSGNCDVVIGIVNNTVYDPLQPELDSFKEEL |
| | | DKYFKNHTSPDVDLGDISGINASVVNIQKEIDRLNEVAKNLNESLIDLQELGKYEQYIKWPWYIWLGFIAGLIAIVMVTIMLCC |
| | | MTSCCSCLKGCCSCGSCCKFDEDDSEPVLKGVKLHYT |
| 76 | 2A6200 | MFVFLVLLPLVSSQCVNFTNRTQLPSAYTNSFTRGVYYPDKVFRSSVLHSTQDLFLPFFSNVTWFHAIHVSGTNGTKRFDN |
| | | PVLPFNDGVYFASTEKSNIIRGWIFGTTLDSKTQSLLIVNNATNVVIKVCEFQFCNYPFLGVYYHKNNKSWMESEFRVYSSA |
| | | NNCTFEYVSQPFLMDLEGKQGNFKNLSEFVFKNIDGYFKIYSKHTPINLVRDLPQGFSALEPLVDLPIGINITRFQTLLALHRS |
| | | YLTPGDSSSGWTAGAAAYYVGYLQPRTFLLKYNENGTITDAVDCALDPLSETKCTLKSFTVEKGIYQTSNFRVQPTESIVRF |
| | | PNITNLCPFGEVFNATRFASVYAWNRKRISNCVADYSVLYNSASFSTFKCYGVSPTKLNDLCFTNVYADSFVIRGDEVRQIA |
| | | PGQTGTIADYNYKLPDDFTGCVIAWNSNNLDSKVGGNYNYLYRLFRKSNLKPFERDISTEIYQAGSTPCNGVKGFNCYFPL |
| | | QSYGFQPTYGVGYQPYRVVVLSFELLHAPATVCGPKKSTNLVKNKCVNFNFNGLTGTGVLTESNKKFLPFQQFGRDIADTT |
| | | DAVRDPQTLEILDITPCSFGGVSVITPGTNTSNQVAVLYQGVNCTEVPVAIHADQLTPTWRVYSTGSNVFQTRAGCLIGAEY |
| | | VNNSYECDIPIGAGICASYQTQTNSPRRARSVASQSIIAYTMSLGAENSVAYSNNSIAIPTNFTISVTTEILPVSMTKTSVDCT |
| | | MYICGDSTECSNLLLQYGSFCTQLNRALTGIAVEQDKNTQEVFAQVKQIYKTPPIKDFGGFNFSQILPDPSKPSKRSFIEDLL |
| | | FNKVTLADAGFIKQYGDCLGDIAARDLICAQKFNGLTVLPPLLTDEMIAQYTSALLAGTITSGWTFGAGAALQIPFAMQMAYR |
| | | FNGIGVTQNVLYENQKLIANQFNSAIGKIQDSLSSTASALGKLQDVVNQNAQALNTLVKQLSSNFGAISSVLNDILSRLDPPE |
| | | AEVQIDRLITGRLQSLQTYVTQQLIRAAEIRASANLAAIKMSECVLGQSKRVDFCGKGYHLMSFPQSAPHGVVFLHVTYVPA |
| | | QEKNFTTAPAICHDGKAHFPREGVFVSNGTHWFVTQRNFYEPQIITTDNTFVSGNCDVVIGIVNNTVYDPLQPELDSFKEEL |
| | | DKYFKNHTSPDVDLGDISGINASFVNIQKEIDRLNEVAKNLNESLIDLQELGKYEQYIKWPWYIWLGFIAGLIAIVMVTIMLCCM |
| | | TSCCSCLKGCCSCGSCCKFDEDDSEPVLKGVKLHYT |
| 77 | 2A0620 | MFVFLVLLPLVSSQCVNLTTRTQLPPAYTNSFTRGVYYPDKVFRSSVLHSTQDLFLPFFSNVTWFHAIHVSGTNGTKRFANP |
| | | VLPFNDGVYFASTEKSNIIRGWIFGTTLDSKTQSLLIVNNATNVVIKVCEFQFCNDPFLGVYYHKNNKSWMESEFRVYSSAN |
| | | NCTFEYVSQPFLMDLEGKQGNFKNLREFVFKNIDGYFKIYSKHTPINLVRGLPQGFSALEPLVDLPIGINITRFQTLLALHRSY |
| | | LTPGDSSSGWTAGAAAYYVGYLQPRTFLLKYNENGTITDAVDCALDPLSETKCTLKSFTVEKGIYQTSNFRVQPTESIVRFP |
| | | NITNLCPFGEVFNATRFASVYAWNRKRISNCVADYSVLYNSASFSTFKCYGVSPTKLNDLCFTNVYADSFVIRGDEVRQIAP |
| | | GQTGNIADYNYKLPDDFTGCVIAWNSNNLDSKVGGNYNYLYRLFRKSNLKPFERDISTEIYQAGSTPCNGVKGFNCYFPLQ |
| | | SYGFQPTYGVGYQPYRVVVLSFELLHAPATVCGPKKSTNLVKNKCVNFNFNGLTGTGVLTESNKKFLPFQQFGRDIADTTD |
| | | AVRDPQTLEILDITPCSFGGVSVITPGTNTSNQVAVLYQGVNCTEVPVAIHADQLTPTWRVYSTGSNVFQTRAGCLIGAEHV |
| | | NNSYECDIPIGAGICASYQTQTNSPRRARSVASQSIIAYTMSLGVENSVAYSNNSIAIPTNFTISVTTEILPVSMTKTSVDCTM |
| | | YICGDSTECSNLLLQYGSFCTQLNRALTGIAVEQDKNTQEVFAQVKQIYKTPPIKDFGGFNFSQILPDPSKPSKRSFIEDLLF |
| | | NKVTLADAGFIKQYGDCLGDIAARDLICAQKFNGLTVLPPLLTDEMIAQYTSALLAGTITSGWTFGAGAALQIPFAMQMAYRF |
| | | NGIGVTQNVLYENQKLIANQFNSAIGKIQDSLSSTASALGKLQDVVNQNAQALNTLVKQLSSNFGAISSVLNDILSRLDPPEA |
| | | EVQIDRLITGRLQSLQTYVTQQLIRAAEIRASANLAATKMSECVLGQSKRVDFCGKGYHLMSFPQSAPHGVVFLHVTYVPA |
| | | QEKNFTTAPAICHDGKAHFPREGVFVSNGTHWFVTQRNFYEPQIITTDNTFVSGNCDVVIGIVNNTVYDPLQPELDSFKEEL |
| | | DKYFKNHTSPDVDLGDISGINASVVNIQKEIDRLNEVAKNLNESLIDLQELGKYEQYIKWPWYIWLGFIAGLIAIVMVTIMLCC |
| | | MTSCCSCLKGCCSCGSCCKFDEDDSEPVLKGVKLHYT |
| 78 | 2A3430 | MFVFLVLLPLVSSQCVNLTTRTQLPPAYTNSFTRGVYYPDKVFRSSVLHSTQDLFLPFFSNVTWFHAISGTNGTKRFDNPVL |
| | | PFNDGVYFASTEKSNIIRGWIFGTTLDSKTQSLLIVNNATNVVIKVCEFQFCNDPFLGVYHKNNKSWMESEFRVYSSANNCT |
| | | FEYVSQPFLMDLEGKQGNFKNLREFVFKNIDGYFKIYSKHTPINLVRDLPQGFSALEPLVDLPIGINITRFQTLLALHRSYLTP |
| | | GDSSSGWTAGAAAYYVGYLQPRTFLLKYNENGTITDAVDCALDPLSETKCTLKSFTVEKGIYQTSNFRVQPTESIVRFPNIT |
| | | NLCPFGEVFNATRFASVYAWNRKRISNCVADYSVLYNSASFSTFKCYGVSPTKLNDLCFTNVYADSFVIRGDEVRQIAPGQ |
| | | TGKIADYNYKLPDDFTGCVIAWNSNNLDSKVGGNYNYLYRLFRKSNLKPFERDISTEIYQAGSTPCNGVEGFNCYFPLQSY |
| | | GFQPTYGVGYQPYRVVVLSFELLHAPATVCGPKKSTNLVKNKCVNFNFNGLTGTGVLTESNKKFLPFQQFGRDIDDTTDAV |
| | | RDPQTLEILDITPCSFGGVSVITPGTNTSNQVAVLYQGVNCTEVPVAIHADQLTPTWRVYSTGSNVFQTRAGCLIGAEHVNN |
| | | SYECDIPIGAGICASYQTQTNSHRRARSVASQSIIAYTMSLGAENSVAYSNNSIAIPTNFTISVTTEILPVSMTKTSVDCTMYIC |
| | | GDSTECSNLLLQYGSFCIQLNRALTGIAVEQDKNTQEVFAQVKQIYKTPPIKDFGGFNFSQILPDPSKPSKRSFIEDLLFNKVT |
| | | LADAGFIKQYGDCLGDIAARDLICAQKFNGLTVLPPLLTDEMIAQYTSALLAGTITSGWTFGAGAALQIPFAMQMAYRFNGIG |
| | | VTQNVLYENQKLIANQFNSAIGKIQDSLSSTASALGKLQDVVNQNAQALNTLVKQLSSNFGAISSVLNDILARLDPPEAEVQI |
| | | DRLITGRLQSLQTYVTQQLIRAAEIRASANLAATKMSECVLGQSKRVDFCGKGYHLMSFPQSAPHGVVFLHVTYVPAQEKN |
| | | FTTAPAICHDGKAHFPREGVFVSNGTHWFVTQRNFYEPQIITTHNTFVSGNCDVVIGIVNNTVYDPLQPELDSFKEELDKYF |
| | | KNHTSPDVDLGDISGINASVVNIQKEIDRLNEVAKNLNESLIDLQELGKYEQYIKWPWYIWLGFIAGLIAIVMVTIMLCCMTSC |
| | | CSCLKGCCSCGSCCKFDEDDSEPVLKGVKLHYT |
| 79 | 2A8210 | MGVKVLFALICIAVAEAKRVQPTESIVRFPNITNLCPFGEVFNATRFASVYAWNRKRISNCVADYSVLYNSASFSTFKCYGVS |
| | | PTKLNDLCFTNVYADSFVIRGDEVRQIAPGQTGNIADYNYKLPDDFTGCVIAWNSNNLDSKVGGNYNYLYRLFRKSNLKPF |
| | | ERDISTEIYQAGSTPCNGVKGFNCYFPLQSYGFQPTYGVGYQPYRVVVLSFELLHAPATVCGPKKSTNLVKNKCVNF |
| 80 | 2A9200 | MFVFLVLLPLVSSQCVNFTTRTQLPPAYTNSFTRGVYYPDKVFRSSVLHSTQDLFLPFFSNVTWFHAIHVSGTNGTKRFAN |
| | | PVLPFNDGVYFASTEKSNIIRGWIFGTTLDSKTQSLLIVNNATNVVIKVCEFQFCNDPFLGVYYHKNNKSWMESEFRVYSSA |
| | | NNCTFEYVSQPFLMDLEGKQGNFKNLREFVFKNIDGYFKIYSKHTPINLVRGLPQGFSALEPLVDLPIGINITRFQTLHISYLT |
| | | PGDSSSGWTAGAAAYYVGYLQPRTFLLKYNENGTITDAVDCALDPLSETKCTLKSFTVEKGIYQTSNFRVQPTESIVRFPNI |
| | | TNLCPFGEVFNATRFASVYAWNRKRISNCVADYSVLYNSASFSTFKCYGVSPTKLNDLCFTNVYADSFVIRGDEVRQIAPG |
| | | QTGNIADYNYKLPDDFTGCVIAWNSNNLDSKVGGNYNYLYRLFRKSNLKPFERDISTEIYQAGSTPCNGVKGFNCYFPLQS |
| | | YGFQPTYGVGYQPYRVVVLSFELLHAPATVCGPKKSTNLVKNKCVNFNFNGLTGTGVLTESNKKFLPFQQFGRDIADTTDA |
| | | VRDPQTLEILDITPCSFGGVSVITPGTNTSNQVAVLYQGVNCTEVPVAIHADQLTPTWRVYSTGSNVFQTRAGCLIGAEHVN |
| | | NSYECDIPIGAGICASYQTQTNSPRRARSVASQSIIAYTMSLGVENSVAYSNNSIAIPTNFTISVTTEILPVSMTKTSVDCTMYI |
| | | CGDSTECSNLLLQYGSFCTQLNRALTGIAVEQDKNTQEVFAQVKQIYKTPPIKDFGGFNFSQILPDPSKPSKRSFIEDLLFNK |
| | | VTLADAGFIKQYGDCLGDIAARDLICAQKFNGLTVLPPLLTDEMIAQYTSALLAGTITSGWTFGAGAALQIPFAMQMAYRFNG |
| | | IGVTQNVLYENQKLIANQFNSAIGKIQDSLSSTASALGKLQDVVNQNAQALNTLVKQLSSNFGAISSVLNDILSRLDPPEAEV |
| | | QIDRLITGRLQSLQTYVTQQLIRAAEIRASANLAATKMSECVLGQSKRVDFCGKGYHLMSFPQSAPHGVVFLHVTYVPAQE |
| | | KNFTTAPAICHDGKAHFPREGVFVSNGTHWFVTQRNFYEPQIITTDNTFVSGNCDVVIGIVNNTVYDPLQPELDSFKEELDK |
| | | YFKNHTSPDVDLGDISGINASVVNIQKEIDRLNEVAKNLNESLIDLQELGKYEQYIKWPWYIWLGFIAGLIAIVMVTIMLCCMTS |
| | | CCSCLKGCCSCGSCCKFDEDDSEPVLKGVKLHYT |
| 81 | 2A4361 | MGVKVLFALICIAVAEAKRVQPTESIVRFPNITNLCPFGEVFNATRFASVYAWNRKRISNCVADYSVLYNSASFSTFKCYGVS |
| | | PTKLNDLCFTNVYADSFVIRGDEVRQIAPGQTGKIADYNYKLPDDFTGCVIAWNSNNLDSKVGGNYNYLYRLFRKSNLKPFE |
| | | RDISTEIYQAGSTPCNGVEGENCYFPLQSYGFQPTNGVGYQPYRVVVLSFELLHAPATVCGPKKSTNLVKNKCVNFGSGR |
| | | LRRGSGATNFSLLKQAGDVEENPGPMGVKVLFALICIAVAEAKRVQPTESIVRFPNITNLCPFGEVFNATRFASVYAWNRKR |
| | | ISNCVADYSVLYNSASFSTFKCYGVSPTKLNDLCFTNVYADSFVIRGDEVRQIAPGQTGKIADYNYKLPDDFTGCVIAWNSN |
| | | NLDSKVGGNYNYLYRLFRKSNLKPFERDISTEIYQAGSTPCNGVEGENCYFPLQSYGFQPTNGVGYQPYRVVVLSFELLHA |
| | | PATVCGPKKSTNLVKNKCVNF |
| 82 | 2A5361 | MGVKVLFALICIAVAEAKRVQPTESIVRFPNITNLCPFGEVFNATRFASVYAWNRKRISNCVADYSVLYNSASFSTFKCYGVS |
| | | PTKLNDLCFTNVYADSFVIRGDEVRQIAPGQTGKIADYNYKLPDDFTGCVIAWNSNNLDSKVGGNYNYLYRLFRKSNLKPFE |
| | | RDISTEIYQAGSTPCNGVEGENCYFPLQSYGFQPTNGVGYQPYRVVVLSFELLHAPATVCGPKKSTNLVKNKCVNFGSGR |
| | | LRRGSGATNFSLLKQAGDVEENPGPMGVKVLFALICIAVAEAKRVQPTESIVRFPNITNLCPFGEVFNATRFASVYAWNRKR |
| | | ISNCVADYSVLYNSASFSTFKCYGVSPTKLNDLCFTNVYADSFVIRGDEVRQIAPGQTGKIADYNYKLPDDFTGCVIAWNSN |
| | | NLDSKVGGNYNYLYRLFRKSNLKPFERDISTEIYQAGSTPCNGVEGFNCYFPLQSYGFQPTNGVGYQPYRVVVLSFELLHA |
| | | PATVCGPKKSTNLVKNKCVNFGSGRLRRGSGATNFSLLKQAGDVEENPGPMGVKVLFALICIAVAEAKRVQPTESIVRFPNI |
| | | TNLCPFGEVFNATRFASVYAWNRKRISNCVADYSVLYNSASFSTFKCYGVSPTKLNDLCFTNVYADSFVIRGDEVRQIAPG |
| | | QTGKIADYNYKLPDDFTGCVIAWNSNNLDSKVGGNYNYLYRLFRKSNLKPFERDISTEIYQAGSTPCNGVEGENCYFPLQS |
| | | YGFQPTNGVGYQPYRVVVLSFELLHAPATVCGPKKSTNLVKNKCVNF |
| 83 | 2A3461 | MGVKVLFALICIAVAEAKRVQPTESIVRFPNITNLCPFGEVFNATRFASVYAWNRKRISNCVADYSVLYNSASFSTFKCYGVS |
| | | PTKLNDLCFTNVYADSFVIRGDEVRQIAPGQTGKIADYNYKLPDDFTGCVIAWNSNNLDSKVGGNYNYLYRLFRKSNLKPFE |
| | | RDISTEIYQAGSTPCNGVEGFNCYFPLQSYGFQPTNGVGYQPYRVVVLSFELLHAPATVCGPKKSTNLVKNKCVNFGSGR |
| | | LRRGSGATNFSLLKQAGDVEENPGPRVQPTESIVRFPNITNLCPFGEVFNATRFASVYAWNRKRISNCVADYSVLYNSASF |
| | | STFKCYGVSPTKLNDLCFTNVYADSFVIRGDEVRQIAPGQTGKIADYNYKLPDDFTGCVIAWNSNNLDSKVGGNYNYLYRL |
| | | FRKSNLKPFERDISTEIYQAGSTPCNGVEGFNCYFPLQSYGFQPTNGVGYQPYRVVVLSFELLHAPATVCGPKKSTNLVKN |
| | | KCVNF |
| 84 | 2A4461 | MGVKVLFALICIAVAEAKRVQPTESIVRFPNITNLCPFGEVFNATRFASVYAWNRKRISNCVADYSVLYNSASFSTFKCYGVS |
| | | PTKLNDLCFTNVYADSFVIRGDEVRQIAPGQTGKIADYNYKLPDDFTGCVIAWNSNNLDSKVGGNYNYLYRLFRKSNLKPFE |
| | | RDISTEIYQAGSTPCNGVEGFNCYFPLQSYGFQPTNGVGYQPYRVVVLSFELLHAPATVCGPKKSTNLVKNKCVNFGSGR |
| | | LRRGSGATNFSLLKQAGDVEENPGPRVQPTESIVRFPNITNLCPFGEVFNATRFASVYAWNRKRISNCVADYSVLYNSASF |
| | | STFKCYGVSPTKLNDLCFTNVYADSFVIRGDEVRQIAPGQTGKIADYNYKLPDDFTGCVIAWNSNNLDSKVGGNYNYLYRL |
| | | FRKSNLKPFERDISTEIYQAGSTPCNGVEGFNCYFPLQSYGFQPTNGVGYQPYRVVVLSFELLHAPATVCGPKKSTNLVKN |
| | | KCVNFGSGRLRRGSGATNFSLLKQAGDVEENPGPRVQPTESIVRFPNITNLCPFGEVFNATRFASVYAWNRKRISNCVAD |
| | | YSVLYNSASFSTFKCYGVSPTKLNDLCFTNVYADSFVIRGDEVRQIAPGQTGKIADYNYKLPDDFTGCVIAWNSNNLDSKVG |
| | | GNYNYLYRLFRKSNLKPFERDISTEIYQAGSTPCNGVEGFNCYFPLQSYGFQPTNGVGYQPYRVVVLSFELLHAPATVCGP |
| | | KKSTNLVKNKCVNF |
| 85 | 2A5461 | MGVKVLFALICIAVAEAKRVQPTESIVRFPNITNLCPFGEVFNATRFASVYAWNRKRISNCVADYSVLYNSASFSTFKCYGVS |
| | | PTKLNDLCFTNVYADSFVIRGDEVRQIAPGQTGKIADYNYKLPDDFTGCVIAWNSNNLDSKVGGNYNYLYRLFRKSNLKPFE |
| | | RDISTEIYQAGSTPCNGVEGFNCYFPLQSYGFQPTNGVGYQPYRVVVLSFELLHAPATVCGPKKSTNLVKNKCVNFGSGR |
| | | LRRGSGATNFSLLKQAGDVEENPGPGLEQLESIINFEKLTEWTSGLEQLESIINFEKLTEWTSGLEQLESIINFEKLTEWTS |
| 86 | 2A6461 | MGVKVLFALICIAVAEAKRVQPTESIVRFPNITNLCPFGEVFNATRFASVYAWNRKRISNCVADYSVLYNSASFSTFKCYGVS |
| | | PTKLNDLCFTNVYADSFVIRGDEVRQIAPGQTGKIADYNYKLPDDFTGCVIAWNSNNLDSKVGGNYNYLYRLFRKSNLKPFE |
| | | RDISTEIYQAGSTPCNGVEGFNCYFPLQSYGFQPTNGVGYQPYRVVVLSFELLHAPATVCGPKKSTNLVKNKCVNFRRKR |
| | | RSGSGATNFSLLKQAGDVEENPGPMGVKVLFALICIAVAEAKRVQPTESIVRFPNITNLCPFGEVFNATRFASVYAWNRKRI |
| | | SNCVADYSVLYNSASFSTFKCYGVSPTKLNDLCFTNVYADSFVIRGDEVRQIAPGQTGKIADYNYKLPDDFTGCVIAWNSN |
| | | NLDSKVGGNYNYLYRLFRKSNLKPFERDISTEIYQAGSTPCNGVEGFNCYFPLQSYGFQPTNGVGYQPYRVVVLSFELLHA |
| | | PATVCGPKKSTNLVKNKCVNF |
| 87 | 2A7020 | MFVFLVLLPLVSSQCVNLTTRTQLPPAYTNSFTRGVYYPDKVFRSSVLHSTQDLFLPFFSNVTWFHAIHVSGTNGTKRFDN |
| | | PVLPFNDGVYFASTEKSNIIRGWIFGTTLDSKTQSLLIVNNATNVVIKVCEFQFCNDPFLGVYYHKNNKSWMESEFRVYSSA |
| | | NNCTFEYVSQPFLMDLEGKQGNFKNLREFVFKNIDGYFKIYSKHTPINLVRDLPQGFSALEPLVDLPIGINITRFQTLLALHRS |
| | | YLTPGDSSSGWTAGAAAYYVGYLQPRTFLLKYNENGTITDAVDCALDPLSETKCTLKSFTVEKGIYQTSNFRVQPTESIVRF |
| | | PNITNLCPFGEVFNATRFASVYAWNRKRISNCVADYSVLYNSASFSTFKCYGVSPTKLNDLCFTNVYADSFVIRGDEVRQIA |
| | | PGQTGKIADYNYKLPDDFTGCVIAWNSNNLDSKVGGNYNYLYRLFRKSNLKPFERDISTEIYQAGSTPCNGVEGFNCYFPL |
| | | QSYGFQPTNGVGYQPYRVVVLSFELLHAPATVCGPKKSTNLVKNKCVNFNFNGLTGTGVLTESNKKFLPFQQFGRDIADTT |
| | | DAVRDPQTLEILDITPCSFGGVSVITPGTNTSNQVAVLYQDVNCTEVPVAIHADQLTPTWRVYSTGSNVFQTRAGCLIGAEH |
| | | VNNSYECDIPIGAGICASYQTQTNSPRRARSVASQSIIAYTMSLGAENSVAYSNNSIAIPTNFTISVTTEILPVSMTKTSVDCT |
| | | MYICGDSTECSNLLLQYGSFCTQLNRALTGIAVEQDKNTQEVFAQVKQIYKTPPIKDFGGFNFSQILPDPSKPSKRSFIEDLL |
| | | FNKVTLADAGFIKQYGDCLGDIAARDLICAQKFNGLTVLPPLLTDEMIAQYTSALLAGTITSGWTFGAGAALQIPFAMQMAYR |
| | | FNGIGVTQNVLYENQKLIANQFNSAIGKIQDSLSSTASALGKLQDVVNQNAQALNTLVKQLSSNFGAISSVLNDILSRLDPPE |
| | | AEVQIDRLITGRLQSLQTYVTQQLIRAAEIRASANLAATKMSECVLGQSKRVDFCGKGYHLMSFPQSAPHGVVFLHVTYVPA |
| | | QEKNFTTAPAICHDGKAHFPREGVFVSNGTHWFVTQRNFYEPQIITTDNTFVSGNCDVVIGIVNNTVYDPLQPELDSFKEEL |
| | | DKYFKNHTSPDVDLGDISGINASVVNIQKEIDRLNEVAKNLNESLIDLQELGKYEQYIKWPWYIWLGFIAGLIAIVMVTIMLCC |
| | | MTSCCSCLKGCCSCGSCCKFDEDDSEPVLKGVKLHYT |
| 88 | 2A8620 | MFVFLVLLPLVSSQCVNLTTRTQLPPAYTNSFTRGVYYPDKVFRSSVLHSTQDLFLPFFSNVTWFHAIHVSGTNGTKRFDN |
| | | PVLPFNDGVYFASTEKSNIIRGWIFGTTLDSKTQSLLIVNNATNVVIKVCEFQFCNDPFLGVYYHKNNKSWMESEFRVYSSA |
| | | NNCTFEYVSQPFLMDLEGKQGNFKNLREFVFKNIDGYFKIYSKHTPINLVRDLPQGFSALEPLVDLPIGINITRFQTLLALHRS |
| | | YLTPGDSSSGWTAGAAAYYVGYLQPRTFLLKYNENGTITDAVDCALDPLSETKCTLKSFTVEKGIYQTSNFRVQPTESIVRF |
| | | PNITNLCPFGEVFNATRFASVYAWNRKRISNCVADYSVLYNSASFSTFKCYGVSPTKLNDLCFTNVYADSFVIRGDEVRQIA |
| | | PGQTGKIADYNYKLPDDFTGCVIAWNSNNLDSKVGGNYNYLYRLFRKSNLKPFERDISTEIYQAGSTPCNGVEGFNCYFPL |
| | | QSYGFQPTNGVGYQPYRVVVLSFELLHAPATVCGPKKSTNLVKNKCVNFNFNGLTGTGVLTESNKKFLPFQQFGRDIADTT |
| | | DAVRDPQTLEILDITPCSFGGVSVITPGTNTSNQVAVLYQDVNCTEVPVAIHADQLTPTWRVYSTGSNVFQTRAGCLIGAEH |
| | | VNNSYECDIPIGAGICASYQTQTNSPRRARSVASQSIIAYTMSLGAENSVAYSNNSIAIPTNFTISVTTEILPVSMTKTSVDCT |
| | | MYICGDSTECSNLLLQYGSFCTQLNRALTGIAVEQDKNTQEVFAQVKQIYKTPPIKDFGGFNFSQILPDPSKPSKRSFIEDLL |
| | | FNKVTLADAGFIKQYGDCLGDIAARDLICAQKFNGLTVLPPLLTDEMIAQYTSALLAGTITSGWTFGAGAALQIPFAMQMAYR |
| | | FNGIGVTQNVLYENQKLIANQFN89SAIGKIQDSLSSTASALGKLQDVVNQNAQALNTLVKQLSSNFGAISSVLNDILSRLDPP |
| | | EAEVQIDRLITGRLQSLQTYVTQQLIRAAEIRASANLAATKMSECVLGQSKRVDFCGKGYHLMSFPQSAPHGVVFLHVTYVP |
| | | AQEKNFTTAPAICHDGKAHFPREGVFVSNGTHWFVTQRNFYEPQIITTDNTFVSGNCDVVIGIVNNTVYDPLQPELDSFKEE |
| | | LDKYFKNHTSPDVDLGDISGINASVVNIQKEIDRLNEVAKNLNESLIDLQELGKYEQYIKWPWYIWLGFIAGLIAIVMVTIMLCC |
| | | MTSCCSCLKGCCSCGSCCKFDEDDSEPVLKGVKLHYT |
| 89 | 2A9420 | MFVFLVLLPLVSSQCVNLTTRTQLPPAYTNSFTRGVYYPDKVFRSSVLHSTQDLFLPFFSNVTWFHAIHVSGTNGTKRFDN |
| | | PVLPFNDGVYFASTEKSNIIRGWIFGTTLDSKTQSLLIVNNATNVVIKVCEFQFCNDPFLGVYYHKNNKSWMESEFRVYSSA |
| | | NNCTFEYVSQPFLMDLEGKQGNFKNLREFVFKNIDGYFKIYSKHTPINLVRDLPQGFSALEPLVDLPIGINITRFQTLLALHRS |
| | | YLTPGDSSSGWTAGAAAYYVGYLQPRTFLLKYNENGTITDAVDCALDPLSETKCTLKSFTVEKGIYQTSNFRVQPTESIVRF |
| | | PNITNLCPFGEVFNATRFASVYAWNRKRISNCVADYSVLYNSASFSTFKCYGVSPTKLNDLCFTNVYADSFVIRGDEVRQIA |
| | | PGQTGKIADYNYKLPDDFTGCVIAWNSNNLDSKVGGNYNYLYRLFRKSNLKPFERDISTEIYQAGSTPCNGVEGENCYFPL |
| | | QSYGFQPTNGVGYQPYRVVVLSFELLHAPATVCGPKKSTNLVKNKCVNFNFNGLTGTGVLTESNKKFLPFQQFGRDIADTT |
| | | DAVRDPQTLEILDITPCSFGGVSVITPGTNTSNQVAVLYQDVNCTEVPVAIHADQLTPTWRVYSTGSNVFQTRAGCLIGAEH |
| | | VNNSYECDIPIGAGICASYQTQTNSPRRARSVASQSIIAYTMSLGAENSVAYSNNSIAIPTNFTISVTTEILPVSMTKTSVDCT |
| | | MYICGDSTECSNLLLQYGSFCTQLNRALTGIAVEQDKNTQEVFAQVKQIYKTPPIKDFGGFNFSQILPDPSKPSKRSFIEDLL |
| | | FNKVTLADAGFIKQYGDCLGDIAARDLICAQKFNGLTVLPPLLTDEMIAQYTSALLAGTITSGWTFGAGAALQIPFAMQMAYR |
| | | FNGIGVTQNVLYENQKLIANQFNSAIGKIQDSLSSTASALGKLQDVVNQNAQALNTLVKQLSSNFGAISSVLNDILSRLDPPE |
| | | AEVQIDRLITGRLQSLQTYVTQQLIRAAEIRASANLAATKMSECVLGQSKRVDFCGKGYHLMSFPQSAPHGVVFLHVTYVPA |
| | | QEKNFTTAPAICHDGKAHFPREGVFVSNGTHWFVTQRNFYEPQIITTDNTFVSGNCDVVIGIVNNTVYDPLQPELDSFKEEL |
| | | DKYFKNHTSPDVDLGDISGINASVVNIQKEIDRLNEVAKNLNESLIDLQELGKYEQYIKWPWYIWLGFIAGLIAIVMVTIMLCC |
| | | MTSCCSCLKGCCSCGSCCKFDEDDSEPVLKGVKLHYT |
| 90 | 2A6481 | MFVFLVLLPLVSSQCVNLTTRTQLPPAYTNSFTRGVYYPDKVFRSSVLHSTQDLFLPFFSNVTWFHAIHVSGTNGTKRFDN |
| | | PVLPFNDGVYFASTEKSNIIRGWIFGTTLDSKTQSLLIVNNATNVVIKVCEFQFCNDPFLGVYYHKNNKSWMESEFRVYSSA |
| | | NNCTFEYVSQPFLMDLEGKQGNFKNLREFVFKNIDGYFKIYSKHTPINLVRDLPQGFSALEPLVDLPIGINITRFQTLLALHRS |
| | | YLTPGDSSSGWTAGAAAYYVGYLQPRTFLLKYNENGTITDAVDCALDPLSETKCTLKSFTVEKGIYQTSNFRVQPTESIVRF |
| | | PNITNLCPFGEVFNATRFASVYAWNRKRISNCVADYSVLYNSASFSTFKCYGVSPTKLNDLCFTNVYADSFVIRGDEVRQIA |
| | | PGQTGKIADYNYKLPDDFTGCVIAWNSNNLDSKVGGNYNYLYRLFRKSNLKPFERDISTEIYQAGSTPCNGVEGFNCYFPL |
| | | QSYGFQPTNGVGYQPYRVVVLSFELLHAPATVCGPKKSTNLVKNKCVNFNFNGLTGTGVLTESNKKFLPFQQFGRDIADTT |
| | | DAVRDPQTLEILDITPCSFGGVSVITPGTNTSNQVAVLYQDVNCTEVPVAIHADQLTPTWRVYSTGSNVFQTRAGCLIGAEH |
| | | VNNSYECDIPIGAGICASYQTQTNSPRRARSVASQSIIAYTMSLGAENSVAYSNNSIAIPTNFTISVTTEILPVSMTKTSVDCT |
| | | MYICGDSTECSNLLLQYGSFCTQLNRALTGIAVEQDKNTQEVFAQVKQIYKTPPIKDFGGFNFSQILPDPSKPSKRSFIEDLL |
| | | FNKVTLADAGFIKQYGDCLGDIAARDLICAQKFNGLTVLPPLLTDEMIAQYTSALLAGTITSGWTFGAGAALQIPFAMQMAYR |
| | | FNGIGVTQNVLYENQKLIANQFNSAIGKIQDSLSSTASALGKLQDVVNQNAQALNTLVKQLSSNFGAISSVLNDILSRLDKVE |
| | | AEVQIDRLITGRLQSLQTYVTQQLIRAAEIRASANLAATKMSECVLGQSKRVDFCGKGYHLMSFPQSAPHGVVFLHVTYVPA |
| | | QEKNFTTAPAICHDGKAHFPREGVFVSNGTHWFVTQRNFYEPQIITTDNTFVSGNCDVVIGIVNNTVYDPLQPELDSFKEEL |
| | | DKYFKNHTSPDVDLGDISGINASVVNIQKEIDRLNEVAKNLNESLIDLQELGKYEQYIKWPWYIWLGFIAGLIAIVMVTIMLCC |
| | | MTSCCSCLKGCCSCGSCCKFDEDDSEPVLKGVKLHYT |
| 91 | Spike_ | MAISGVPVLGFFIIAVLMSAQESWAGIISRKPQPKMAQVSSSRRGVYYNDDIFRSDVLHLTQDYFLPFDSNLTQYFSLN |
| | chimera1- | VDSDRYTYFDNPILDFGDGVYFAATEKSNVIRGWIFGSSFDNTTQSAVIVNNSTHIIIRVCNFNLCKEPMYTVSRGTQQ |
| | RBD:SARS- | NAWVYQSAFNCTYDRVEKSFQLDTTPKTGNFKDLREYVFKNRDGFLSVYQTYTAVNLPRGLPTGFSVLKPILKLPFGI |
| | CoV- | NITSYRVVMAMFSQTTSNFLPESAAYYVGNLKYSTFMLRFNENGTITDAVDCSQNPLAELKCTIKNFTVEKGIYQTSN |
| | NTD:HKU3-1- | FRVQPTESIVRFPNITNLCPFGEVFNATKFPSVYAWERKKISNCVADYSVLYNSTFFSTFKCYGVSATKLNDLCFSNVY |
| | S2:SARS- | ADSFVVKGDDVRQIAPGQTGVIADYNYKLPDDFMGCVLAWNTRNIDATSTGNYNYKYRYLRHGKLRPFERDISNVPF |
| | CoV-2 | SPDGKPCTPPALNCYWPLNDYGFYTTTGIGYQPYRVVVLSFELLNAPATVCGPKKSTNLVKNKCVNFNFNGLTGTGV |
| | | LTESNKKFLPFQQFGRDIADTTDAVRDPQTLEILDITPCSFGGVSVITPGTNTSNQVAVLYQDVNCTEVPVAIHADQLT |
| | | PTWRVYSTGSNVFQTRAGCLIGAEHVNNSYECDIPIGAGICASYQTQTNSPRRARSVASQSIIAYTMSLGAENSVAYSN |
| | | NSIAIPTNFTISVTTEILPVSMTKTSVDCTMYICGDSTECSNLLLQYGSFCTQLNRALTGIAVEQDKNTQEVFAQVKQIY |
| | | KTPPIKDFGGFNFSQILPDPSKPSKRSFIEDLLFNKVTLADAGFIKQYGDCLGDIAARDLICAQKFNGLTVLPPLLTDEMI |
| | | AQYTSALLAGTITSGWTFGAGAALQIPFAMQMAYRFNGIGVTQNVLYENQKLIANQFNSAIGKIQDSLSSTASALGKL |
| | | QDVVNQNAQALNTLVKQLSSNFGAISSVLNDILSRLDKVEAEVQIDRLITGRLQSLQTYVTQQLIRAAEIRASANLAAT |
| | | KMSECVLGQSKRVDFCGKGYHLMSFPQSAPHGVVFLHVTYVPAQEKNFTTAPAICHDGKAHFPREGVFVSNGTHWF |
| | | VTQRNFYEPQIITTDNTFVSGNCDVVIGIVNNTVYDPLQPELDSFKEELDKYFKNHTSPDVDLGDISGINASVVNIQKEI |
| | | DRLNEVAKNLNESLIDLQELGKYEQYIKWPWYIWLGFIAGLIAIVMVTIMLCCMTSCCSCLKGCCSCGSCCKFDEDDS |
| | | EPVLKGVKLHYT |
| 92 | Spike_ | MAISGVPVLGFFIIAVLMSAQESWASDLDRCTTFDDVQAPNYTQHTSSMRGVYYPDEIFRSDTLYLTQDLFLPFYSNV |
| | chimera 2- | TGFHTINHTFGNPVIPFKDGIYFAATEKSNVVRGWVFGSTMNNKSQSVIIINNSTNVVIRACNFELCDNPFFAVSKPMGT |
| | RBD:SARS- | QTHTMIFDNAFNCTFEYISDAFSLDVSEKSGNFKHLREFVFKNKDGFLYVYKGYQPIDVVRDLPSGFNTLKPIFKLPLGI |
| | CoV-2- | NITNFRAILTAFSPAQDIWGTSAAAYFVGYLKPTTFMLKYDENGTITDAVDCSQNPLAELKCSVKSFEIDKGIYQTSNF |
| | NTD:SARS- | RVVPSGDVVRFPNITNLCPFGEVFNATRFASVYAWNRKRISNCVADYSVLYNSASFSTFKCYGVSPTKLNDLCFTNVY |
| | CoV, | ADSFVIRGDEVRQIAPGQTGKIADYNYKLPDDFTGCVIAWNSNNLDSKVGGNYNYLYRLFRKSNLKPFERDISTEIYQA |
| | S2:SARS- | GSTPCNGVEGFNCYFPLQSYGFQPTNGVGYQPYRVVVLSFELLHAPATVCGPKLSTDLIKNQCVNFNFNGLTGTGVLT |
| | CoV | PSSKRFQPFQQFGRDVSDFTDSVRDPKTSEILDISPCSFGGVSVITPGTNASSEVAVLYQDVNCTDVSTAIHADQLTPAW |
| | | RIYSTGNNVFQTQAGCLIGAEHVDTSYECDIPIGAGICASYHTVSLLRSTSQKSIVAYTMSLGADSSIAYSNNTIAIPTNF |
| | | SISITTEVMPVSMAKTSVDCNMYICGDSTECANLLLQYGSFCTQLNRALSGIAAEQDRNTREVFAQVKQMYKTPTLKY |
| | | FGGFNFSQILPDPLKPTKRSFIEDLLFNKVTLADAGFMKQYGECLGDINARDLICAQKFNGLTVLPPLLTDDMIAAYTA |
| | | ALVSGTATAGWTFGAGAALQIPFAMQMAYRFNGIGVTQNVLYENQKQIANQFNKAISQIQESLTTTSTALGKLQDVV |
| | | NQNAQALNTLVKQLSSNFGAISSVLNDILSRLDKVEAEVQIDRLITGRLQSLQTYVTQQLIRAAEIRASANLAATKMSE |
| | | CVLGQSKRVDFCGKGYHLMSFPQAAPHGVVFLHVTYVPSQERNFTTAPAICHEGKAYFPREGVFVFNGTSWFITQRNF |
| | | FSPQIITTDNTFVSGNCDVVIGIINNTVYDPLQPELDSFKEELDKYFKNHTSPDVDLGDISGINASVVNIQKEIDRLNEVA |
| | | KNLNESLIDLQELGKYEQYIKWPWYVWLGFIAGLIAIVMVTILLCCMTSCCSCLKGACSCGSCCKFDEDDSEPVLKGV |
| | | KLHYT |
| 93 | Spike_ | MAISGVPVLGFFIIAVLMSAQESWAVNLTTRTQLPPAYTNSFTRGVYYPDKVFRSSVLHSTQDLFLPFFSNVTWFHAIH |
| | chimera 3- | VSGTNGTKRFDNPVLPFNDGVYFASTEKSNIIRGWIFGTTLDSKTQSLLIVNNATNVVIKVCEFQFCNDPFLGVYYHKN |
| | RBD:SARS- | NKSWMESEFRVYSSANNCTFEYVSQPFLMDLEGKQGNFKNLREFVFKNIDGYFKIYSKHTPINLVRDLPQGFSALEPL |
| | CoV- | VDLPIGINITRFQTLLALHRSYLTPGDSSSGWTAGAAAYYVGYLQPRTFLLKYNENGTITDAVDCALDPLSETKCTLKS |
| | NTD:SARS- | FTVEKGIYQTSNFRVQPTESIVRFPNITNLCPFGEVFNATKFPSVYAWERKKISNCVADYSVLYNSTFFSTFKCYGVSAT |
| | CoV2, | KLNDLCFSNVYADSFVVKGDDVRQIAPGQTGVIADYNYKLPDDFMGCVLAWNTRNIDATSTGNYNYKYRYLRHGKL |
| | S2:SARS- | RPFERDISNVPFSPDGKPCTPPALNCYWPLNDYGFYTTTGIGYQPYRVVVLSFELLNAPATVCGPKKSTNLVKNKCVN |
| | CoV2 | FNFNGLTGTGVLTESNKKFLPFQQFGRDIADTTDAVRDPQTLEILDITPCSFGGVSVITPGTNTSNQVAVLYQDVNCTE |
| | | VPVAIHADQLTPTWRVYSTGSNVFQTRAGCLIGAEHVNNSYECDIPIGAGICASYQTQTNSPRRARSVASQSIIAYTMSL |
| | | GAENSVAYSNNSIAIPTNFTISVTTEILPVSMTKTSVDCTMYICGDSTECSNLLLQYGSFCTQLNRALTGIAVEQDKNTQ |
| | | EVFAQVKQIYKTPPIKDFGGFNFSQILPDPSKPSKRSFIEDLLFNKVTLADAGFIKQYGDCLGDIAARDLICAQKENGLT |
| | | VLPPLLTDEMIAQYTSALLAGTITSGWTFGAGAALQIPFAMQMAYRFNGIGVTQNVLYENQKLIANQFNSAIGKIQDS |
| | | LSSTASALGKLQDVVNQNAQALNTLVKQLSSNFGAISSVLNDILSRLDKVEAEVQIDRLITGRLQSLQTYVTQQLIRAA |
| | | EIRASANLAATKMSECVLGQSKRVDFCGKGYHLMSFPQSAPHGVVFLHVTYVPAQEKNFTTAPAICHDGKAHFPREG |
| | | VFVSNGTHWFVTQRNFYEPQIITTDNTFVSGNCDVVIGIVNNTVYDPLQPELDSFKEELDKYFKNHTSPDVDLGDISGI |
| | | NASVVNIQKEIDRLNEVAKNLNESLIDLQELGKYEQYIKWPWYIWLGFIAGLIAIVMVTIMLCCMTSCCSCLKGCCSCG |
| | | SCCKFDEDDSEPVLKGVKLHYT |
| 94 | Spike_ | MAISGVPVLGFFIIAVLMSAQESWAVNLTTRTQLPPAYTNSFTRGVYYPDKVFRSSVLHSTQDLFLPFFSNVTWFHAIH |
| | chimera 4- | VSGTNGTKRFDNPVLPFNDGVYFASTEKSNIIRGWIFGTTLDSKTQSLLIVNNATNVVIKVCEFQFCNDPFLGVYYHKN |
| | RBD: | NKSWMESEFRVYSSANNCTFEYVSQPFLMDLEGKQGNFKNLREFVFKNIDGYFKIYSKHTPINLVRDLPQGFSALEPL |
| | RsSHC014- | VDLPIGINITRFQTLLALHRSYLTPGDSSSGWTAGAAAYYVGYLQPRTFLLKYNENGTITDAVDCALDPLSETKCTLKS |
| | NTD:SARS- | FTVEKGIYQTSNFRVQPTESIVRFPNITNLCPFGEVFNATTFPSVYAWERKRISNCVADYSVLYNSTSFSTFKCYGVSAT |
| | CoV2, | KLNDLCFSNVYADSFVVKGDDVRQIAPGQTGVIADYNYKLPDDFLGCVLAWNTNSKDSSTSGNYNYLYRWVRRSKL |
| | S2:SARS- | NPYERDLSNDIYSPGGQSCSAVGPNCYNPLRPYGFFTTAGVGHQPYRVVVLSFELLNAPATVCGPKKSTNLVKNKCVN |
| | CoV2 | FNFNGLTGTGVLTESNKKFLPFQQFGRDIADTTDAVRDPQTLEILDITPCSFGGVSVITPGTNTSNQVAVLYQDVNCTE |
| | | VPVAIHADQLTPTWRVYSTGSNVFQTRAGCLIGAEHVNNSYECDIPIGAGICASYQTQTNSPRRARSVASQSIIAYTMSL |
| | | GAENSVAYSNNSIAIPTNFTISVTTEILPVSMTKTSVDCTMYICGDSTECSNLLLQYGSFCTQLNRALTGIAVEQDKNTQ |
| | | EVFAQVKQIYKTPPIKDFGGFNFSQILPDPSKPSKRSFIEDLLFNKVTLADAGFIKQYGDCLGDIAARDLICAQKFNGLT |
| | | VLPPLLTDEMIAQYTSALLAGTITSGWTFGAGAALQIPFAMQMAYRFNGIGVTQNVLYENQKLIANQFNSAIGKIQDS |
| | | LSSTASALGKLQDVVNQNAQALNTLVKQLSSNFGAISSVLNDILSRLDKVEAEVQIDRLITGRLQSLQTYVTQQLIRAA |
| | | EIRASANLAATKMSECVLGQSKRVDFCGKGYHLMSFPQSAPHGVVFLHVTYVPAQEKNFTTAPAICHDGKAHFPREG |
| | | VFVSNGTHWFVTQRNFYEPQIITTDNTFVSGNCDVVIGIVNNTVYDPLQPELDSFKEELDKYFKNHTSPDVDLGDISGI |
| | | NASVVNIQKEIDRLNEVAKNLNESLIDLQELGKYEQYIKWPWYIWLGFIAGLIAIVMVTIMLCCMTSCCSCLKGCCSCG |
| | | SCCKFDEDDSEPVLKGVKLHYT |
| 95 | IL2 ss | MYRMQLLSCIALSLALVTNSVNLTTRTQLPPAYTNSFTRGVYYPDKVFRSSVLHSTQDLFLPFFSNVTWFHAIHVSGTNGTK |
| | Spike | RFDNPVLPFNDGVYFASTEKSNIIRGWIFGTTLDSKTQSLLIVNNATNVVIKVCEFQFCNDPFLGVYYHKNNKSWMESEFRV |
| | (highGC)- | YSSANNCTFEYVSQPFLMDLEGKQGNFKNLREFVFKNIDGYFKIYSKHTPINLVRDLPQGFSALEPLVDLPIGINITRFQTLLA |
| | Tri | LHRSYLTPGDSSSGWTAGAAAYYVGYLQPRTFLLKYNENGTITDAVDCALDPLSETKCTLKSFTVEKGIYQTSNFRVQPTE |
| | | SIVRFPNITNLCPFGEVFNATRFASVYAWNRKRISNCVADYSVLYNSASFSTFKCYGVSPTKLNDLCFTNVYADSFVIRGDE |
| | | VRQIAPGQTGKIADYNYKLPDDFTGCVIAWNSNNLDSKVGGNYNYLYRLFRKSNLKPFERDISTEIYQAGSTPCNGVEGEN |
| | | CYFPLQSYGFQPTNGVGYQPYRVVVLSFELLHAPATVCGPKKSTNLVKNKCVNFNFNGLTGTGVLTESNKKFLPFQQFGR |
| | | DIADTTDAVRDPQTLEILDITPCSFGGVSVITPGTNTSNQVAVLYQDVNCTEVPVAIHADQLTPTWRVYSTGSNVFQTRAGC |
| | | LIGAEHVNNSYECDIPIGAGICASYQTQTNSPGSASSVASQSIIAYTMSLGAENSVAYSNNSIAIPTNFTISVTTEILPVSMTKT |
| | | SVDCTMYICGDSTECSNLLLQYGSFCTQLNRALTGIAVEQDKNTQEVFAQVKQIYKTPPIKDFGGFNFSQILPDPSKPSKRS |
| | | FIEDLLFNKVTLADAGFIKQYGDCLGDIAARDLICAQKFNGLTVLPPLLTDEMIAQYTSALLAGTITSGWTFGAGAALQIPFAM |
| | | QMAYRFNGIGVTQNVLYENQKLIANQFNSAIGKIQDSLSSTASALGKLQDVVNQNAQALNTLVKQLSSNFGAISSVLNDILSR |
| | | LDPPEAEVQIDRLITGRLQSLQTYVTQQLIRAAEIRASANLAATKMSECVLGQSKRVDFCGKGYHLMSFPQSAPHGVVFLHV |
| | | TYVPAQEKNFTTAPAICHDGKAHFPREGVFVSNGTHWFVTQRNFYEPQIITTDNTFVSGNCDVVIGIVNNTVYDPLQPELDS |
| | | FKEELDKYFKNHTSPDVDLGDISGINASVVNIQKEIDRLNEVAKNLNESLIDLQELGKYEQGYIPEAPRDGQAYVRKDGEWV |
| | | LLSTFL |
| 96 | II-2 ss | MGVKVLFALICIAVAEAKVNLTTRTQLPPAYTNSFTRGVYYPDKVFRSSVLHSTQDLFLPFFSNVTWFHAIHVSGTNGTKRF |
| | Spike | DNPVLPFNDGVYFASTEKSNIIRGWIFGTTLDSKTQSLLIVNNATNVVIKVCEFQFCNDPFLGVYYHKNNKSWMESEFRVYS |
| | (highGC)- | SANNCTFEYVSQPFLMDLEGKQGNFKNLREFVFKNIDGYFKIYSKHTPINLVRDLPQGFSALEPLVDLPIGINITRFQTLLALH |
| | Tri | RSYLTPGDSSSGWTAGAAAYYVGYLQPRTFLLKYNENGTITDAVDCALDPLSETKCTLKSFTVEKGIYQTSNFRVQPTESIV |
| | | RFPNITNLCPFGEVFNATRFASVYAWNRKRISNCVADYSVLYNSASFSTFKCYGVSPTKLNDLCFTNVYADSFVIRGDEVR |
| | | QIAPGQTGKIADYNYKLPDDFTGCVIAWNSNNLDSKVGGNYNYLYRLFRKSNLKPFERDISTEIYQAGSTPCNGVEGENCY |
| | | FPLQSYGFQPTNGVGYQPYRVVVLSFELLHAPATVCGPKKSTNLVKNKCVNFNFNGLTGTGVLTESNKKFLPFQQFGRDIA |
| | | DTTDAVRDPQTLEILDITPCSFGGVSVITPGTNTSNQVAVLYQDVNCTEVPVAIHADQLTPTWRVYSTGSNVFQTRAGCLIG |
| | | AEHVNNSYECDIPIGAGICASYQTQTNSPGSASSVASQSIIAYTMSLGAENSVAYSNNSIAIPTNFTISVTTEILPVSMTKTSVD |
| | | CTMYICGDSTECSNLLLQYGSFCTQLNRALTGIAVEQDKNTQEVFAQVKQIYKTPPIKDFGGFNFSQILPDPSKPSKRSFIED |
| | | LLFNKVTLADAGFIKQYGDCLGDIAARDLICAQKFNGLTVLPPLLTDEMIAQYTSALLAGTITSGWTFGAGAALQIPFAMQMA |
| | | YRFNGIGVTQNVLYENQKLIANQFNSAIGKIQDSLSSTASALGKLQDVVNQNAQALNTLVKQLSSNFGAISSVLNDILSRLDP |
| | | PEAEVQIDRLITGRLQSLQTYVTQQLIRAAEIRASANLAATKMSECVLGQSKRVDFCGKGYHLMSFPQSAPHGVVFLHVTYV |
| | | PAQEKNFTTAPAICHDGKAHFPREGVFVSNGTHWFVTQRNFYEPQIITTDNTFVSGNCDVVIGIVNNTVYDPLQPELDSFKE |
| | | ELDKYFKNHTSPDVDLGDISGINASVVNIQKEIDRLNEVAKNLNESLIDLQELGKYEQGYIPEAPRDGQAYVRKDGEWVLLST |
| | | FL |
| 97 | Gluc ss | MGVKVLFALICIAVAEAKVNLTTRTQLPPAYTNSFTRGVYYPDKVFRSSVLHSTQDLFLPFFSNVTWFHAIHVSGTNGTKRF |
| | Spike- | DNPVLPFNDGVYFASTEKSNIIRGWIFGTTLDSKTQSLLIVNNATNVVIKVCEFQFCNDPFLGVYYHKNNKSWMESEFRVYS |
| | Tri(lowGC) | SANNCTFEYVSQPFLMDLEGKQGNFKNLREFVFKNIDGYFKIYSKHTPINLVRDLPQGFSALEPLVDLPIGINITRFQTLLALH |
| | | RSYLTPGDSSSGWTAGAAAYYVGYLQPRTFLLKYNENGTITDAVDCALDPLSETKCTLKSFTVEKGIYQTSNFRVQPTESIV |
| | | RFPNITNLCPFGEVFNATRFASVYAWNRKRISNCVADYSVLYNSASFSTFKCYGVSPTKLNDLCFTNVYADSFVIRGDEVR |
| | | QIAPGQTGKIADYNYKLPDDFTGCVIAWNSNNLDSKVGGNYNYLYRLFRKSNLKPFERDISTEIYQAGSTPCNGVEGENCY |
| | | FPLQSYGFQPTNGVGYQPYRVVVLSFELLHAPATVCGPKKSTNLVKNKCVNFNFNGLTGTGVLTESNKKFLPFQQFGRDIA |
| | | DTTDAVRDPQTLEILDITPCSFGGVSVITPGTNTSNQVAVLYQDVNCTEVPVAIHADQLTPTWRVYSTGSNVFQTRAGCLIG |
| | | AEHVNNSYECDIPIGAGICASYQTQTNSPGSASSVASQSIIAYTMSLGAENSVAYSNNSIAIPTNFTISVTTEILPVSMTKTSVD |
| | | CTMYICGDSTECSNLLLQYGSFCTQLNRALTGIAVEQDKNTQEVFAQVKQIYKTPPIKDFGGFNFSQILPDPSKPSKRSFIED |
| | | LLFNKVTLADAGFIKQYGDCLGDIAARDLICAQKFNGLTVLPPLLTDEMIAQYTSALLAGTITSGWTFGAGAALQIPFAMQMA |
| | | YRFNGIGVTQNVLYENQKLIANQFNSAIGKIQDSLSSTASALGKLQDVVNQNAQALNTLVKQLSSNFGAISSVLNDILSRLDP |
| | | PEAEVQIDRLITGRLQSLQTYVTQQLIRAAEIRASANLAATKMSECVLGQSKRVDFCGKGYHLMSFPQSAPHGVVFLHVTYV |
| | | PAQEKNFTTAPAICHDGKAHFPREGVFVSNGTHWFVTQRNFYEPQIITTDNTFVSGNCDVVIGIVNNTVYDPLQPELDSFKE |
| | | ELDKYFKNHTSPDVDLGDISGINASVVNIQKEIDRLNEVAKNLNESLIDLQELGKYEQYIKWPWYIWLGFIAGLIAIVMVTIMLC |
| | | CMTSCCSCLKGCCSCGSCCKFDEDDSEPVLKGVKLHYT |
| 98 | WT ss | MGVKVLFALICIAVAEAKRVQPTESIVRFPNITNLCPFGEVFNATRFASVYAWNRKRISNCVADYSVLYNSASFSTFKCYGVS |
| | RBD *- | PTKLNDLCFTNVYADSFVIRGDEVRQIAPGQTGKIADYNYKLPDDFTGCVIAWNSNNLDSKVGGNYNYLYRLFRKSNLKPFE |
| | Tri(lowGC) | RDISTEIYQAGSTPCNGVEGFNCYFPLQSYGFQPTNGVGYQPYRVVVLSFELLHAPATVCGPKKSTNLVKNKCVNFGYIPE |
| | | APRDGQAYVRKDGEWVLLSTFL |
| 99 | Gluc RBD*- | MFVFLVLLPLVSSQCVNLTTRTQLPPAYTNSFTRGVYYPDKVFRSSVLHSTQDLFLPFFSNVTWFHAIHVSGTNGTKRFDN |
| | Tri(lowGC) | PVLPFNDGVYFASTEKSNIIRGWIFGTTLDSKTQSLLIVNNATNVVIKVCEFQFCNDPFLGVYYHKNNKSWMESEFRVYSSA |
| | | NNCTFEYVSQPFLMDLEGKQGNFKNLREFVFKNIDGYFKIYSKHTPINLVRDLPQGFSALEPLVDLPIGINITRFQTLLALHRS |
| | | YLTPGDSSSGWTAGAAAYYVGYLQPRTFLLKYNENGTITDAVDCALDPLSETKCTLKSFTVEKGIYQTSNFRVQPTESIVRF |
| | | PNITNLCPFGEVFNATRFASVYAWNRKRISNCVADYSVLYNSASFSTFKCYGVSPTKLNDLCFTNVYADSFVIRGDEVRQIA |
| | | PGQTGKIADYNYKLPDDFTGCVIAWNSNNLDSKVGGNYNYLYRLFRKSNLKPFERDISTEIYQAGSTPCNGVEGENCYFPL |
| | | QSYGFQPTNGVGYQPYRVVVLSFELLHAPATVCGPKKSTNLVKNKCVNFNFNGLTGTGVLTESNKKFLPFQQFGRDIADTT |
| | | DAVRDPQTLEILDITPCSFGGVSVITPGTNTSNQVAVLYQGVNCTEVPVAIHADQLTPTWRVYSTGSNVFQTRAGCLIGAEH |
| | | VNNSYECDIPIGAGICASYQTQTNSPGSASSVASQSIIAYTMSLGAENSVAYSNNSIAIPTNFTISVTTEILPVSMTKTSVDCT |
| | | MYICGDSTECSNLLLQYGSFCTQLNRALTGIAVEQDKNTQEVFAQVKQIYKTPPIKDFGGFNFSQILPDPSKPSKRSFIEDLL |
| | | FNKVTLADAGFIKQYGDCLGDIAARDLICAQKFNGLTVLPPLLTDEMIAQYTSALLAGTITSGWTFGAGAALQIPFAMQMAYR |
| | | FNGIGVTQNVLYENQKLIANQFNSAIGKIQDSLSSTASALGKLQDVVNQNAQALNTLVKQLSSNFGAISSVLNDILSRLDPPE |
| | | AEVQIDRLITGRLQSLQTYVTQQLIRAAEIRASANLAATKMSECVLGQSKRVDFCGKGYHLMSFPQSAPHGVVFLHVTYVPA |
| | | QEKNFTTAPAICHDGKAHFPREGVFVSNGTHWFVTQRNFYEPQIITTDNTFVSGNCDVVIGIVNNTVYDPLQPELDSFKEEL |
| | | DKYFKNHTSPDVDLGDISGINASVVNIQKEIDRLNEVAKNLNESLIDLQELGKYEQYIKWPWYIWLGFIAGLIAIVMVTIMLCC |
| | | MTSCCSCLKGCCSCGSCCKFDEDDSEPVLKGVKLHYT |
| 100 | 2A2361 | MGVKVLFALICIAVAEAKRVQPTESIVRFPNITNLCPFGEVFNATRFASVYAWNRKRISNCVADYSVLYNSASFSTFKCYGVS |
| | | PTKLNDLCFTNVYADSFVIRGDEVRQIAPGQTGKIADYNYKLPDDFTGCVIAWNSNNLDSKVGGNYNYLYRLFRKSNLKPFE |
| | | RDISTEIYQAGSTPCNGVEGENCYFPLQSYGFQPTNGVGYQPYRVVVLSFELLHAPATVCGPKKSTNLVKNKCVNFGGGG |
| | | SGYIPEAPRDGQAYVRKDGEWVLLSTFL |
| 101 | 2A3361 | MGVKVLFALICIAVAEAKRVQPTESIVRFPNITNLCPFGEVFNATRFASVYAWNRKRISNCVADYSVLYNSASFSTFKCYGVS |
| | | PTKLNDLCFTNVYADSFVIRGDEVRQIAPGQTGKIADYNYKLPDDFTGCVIAWNSNNLDSKVGGNYNYLYRLFRKSNLKPFE |
| | | RDISTEIYQAGSTPCNGVEGFNCYFPLQSYGFQPTNGVGYQPYRVVVLSFELLHAPATVCGPKKSTNLVKNKCVNFGGGG |
| | | SGYIPEAPRDGQAYVRKDGEWVLLSTFLGGGGSRVQPTESIVRFPNITNLCPFGEVFNATRFASVYAWNRKRISNCVADYS |
| | | VLYNSASFSTFKCYGVSPTKLNDLCFTNVYADSFVIRGDEVRQIAPGQTGKIADYNYKLPDDFTGCVIAWNSNNLDSKVGG |
| | | NYNYLYRLFRKSNLKPFERDISTEIYQAGSTPCNGVEGENCYFPLQSYGFQPTNGVGYQPYRVVVLSFELLHAPATVCGPK |
| | | KSTNLVKNKCVNF |
| 102 | 2A6361 | MGVKVLFALICIAVAEAKRVQPTESIVRFPNITNLCPFGEVFNATRFASVYAWNRKRISNCVADYSVLYNSASFSTFKCYGVS |
| | | PTKLNDLCFTNVYADSFVIRGDEVRQIAPGQTGKIADYNYKLPDDFTGCVIAWNSNNLDSKVGGNYNYLYRLFRKSNLKPFE |
| | | RDISTEIYQAGSTPCNGVEGFNCYFPLQSYGFQPTNGVGYQPYRVVVLSFELLHAPATVCGPKKSTNLVKNKCVNFDIIKLL |
| | | NEQVNKEMNSSNLYMSMSSWCYTHSLDGAGLFLFDHAAEEYEHAKKLIVFLNENNVPVQLTSISAPEHKFESLTQIFQKAY |
| | | EHEQHISESINNIVDHAIKGKDHATFNFLQWYVSEQHEEEVLFKDILDKIELIGNENHGLYLADQYVKGIAKSRKS |
| 103 | 2A7361 | MGVKVLFALICIAVAEAKRVQPTESIVRFPNITNLCPFGEVFNATRFASVYAWNRKRISNCVADYSVLYNSASFSTFKCYGVS |
| | | PTKLNDLCFTNVYADSFVIRGDEVRQIAPGQTGKIADYNYKLPDDFTGCVIAWNSNNLDSKVGGNYNYLYRLFRKSNLKPFE |
| | | RDISTEIYQAGSTPCNGVEGFNCYFPLQSYGFQPTNGVGYQPYRVVVLSFELLHAPATVCGPKKSTNLVKNKCVNFGGGG |
| | | SDIIKLLNEQVNKEMNSSNLYMSMSSWCYTHSLDGAGLFLFDHAAEEYEHAKKLIVFLNENNVPVQLTSISAPEHKFESLTQI |
| | | FQKAYEHEQHISESINNIVDHAIKGKDHATFNFLQWYVSEQHEEEVLFKDILDKIELIGNENHGLYLADQYVKGIAKSRKS |
| 104 | 2A8361 | MGVKVLFALICIAVAEAKRVQPTESIVRFPNITNLCPFGEVFNATRFASVYAWNRKRISNCVADYSVLYNSASFSTFKCYGVS |
| | | PTKLNDLCFTNVYADSFVIRGDEVRQIAPGQTGKIADYNYKLPDDFTGCVIAWNSNNLDSKVGGNYNYLYRLFRKSNLKPFE |
| | | RDISTEIYQAGSTPCNGVEGENCYFPLQSYGFQPTNGVGYQPYRVVVLSFELLHAPATVCGPKKSTNLVKNKCVNFPGSG |
| | | YIPEAPRDGQAYVRKDGEWVLLSTFLSGRSGGDIIKLLNEQVNKEMNSSNLYMSMSSWCYTHSLDGAGLFLFDHAAEEYE |
| | | HAKKLIVFLNENNVPVQLTSISAPEHKFESLTQIFQKAYEHEQHISESINNIVDHAIKGKDHATFNFLQWYVSEQHEEEVLFKD |
| | | ILDKIELIGNENHGLYLADQYVKGIAKSRKS |
| 105 | 2A9361 | MGVKVLFALICIAVAEAKRVQPTESIVRFPNITNLCPFGEVFNATRFASVYAWNRKRISNCVADYSVLYNSASFSTFKCYGVS |
| | | PTKLNDLCFTNVYADSFVIRGDEVRQIAPGQTGKIADYNYKLPDDFTGCVIAWNSNNLDSKVGGNYNYLYRLFRKSNLKPFE |
| | | RDISTEIYQAGSTPCNGVEGFNCYFPLQSYGFQPTNGVGYQPYRVVVLSFELLHAPATVCGPKKSTNLVKNKCVNFINHVG |
| | | GTGGAIMAPVAVTRQLVGS |
| 106 | 2A0461 | MGVKVLFALICIAVAEAKRVQPTESIVRFPNITNLCPFGEVFNATRFASVYAWNRKRISNCVADYSVLYNSASFSTFKCYGVS |
| | | PTKLNDLCFTNVYADSFVIRGDEVRQIAPGQTGKIADYNYKLPDDFTGCVIAWNSNNLDSKVGGNYNYLYRLFRKSNLKPFE |
| | | RDISTEIYQAGSTPCNGVEGFNCYFPLQSYGFQPTNGVGYQPYRVVVLSFELLHAPATVCGPKKSTNLVKNKCVNFGGGG |
| | | SINHVGGTGGAIMAPVAVTRQLVGS |
| 107 | 2A1461 | MGVKVLFALICIAVAEAKINHVGGTGGAIMAPVAVTRQLVGSRVQPTESIVRFPNITNLCPFGEVFNATRFASVYAWNRKRIS |
| | | NCVADYSVLYNSASFSTFKCYGVSPTKLNDLCFTNVYADSFVIRGDEVRQIAPGQTGKIADYNYKLPDDFTGCVIAWNSNNL |
| | | DSKVGGNYNYLYRLFRKSNLKPFERDISTEIYQAGSTPCNGVEGENCYFPLQSYGFQPTNGVGYQPYRVVVLSFELLHAPA |
| | | TVCGPKKSTNLVKNKCVNF |
| 108 | 2A2461 | MGVKVLFALICIAVAEAKINHVGGTGGAIMAPVAVTRQLVGSGGGGSRVQPTESIVRFPNITNLCPFGEVFNATRFASVYAW |
| | | NRKRISNCVADYSVLYNSASFSTFKCYGVSPTKLNDLCFTNVYADSFVIRGDEVRQIAPGQTGKIADYNYKLPDDFTGCVIA |
| | | WNSNNLDSKVGGNYNYLYRLFRKSNLKPFERDISTEIYQAGSTPCNGVEGFNCYFPLQSYGFQPTNGVGYQPYRVVVLSF |
| | | ELLHAPATVCGPKKSTNLVKNKCVNF |
| 109 | | MGVKVLFALICIAVAEAKAKFVAAWTLKAAARVQPTESIVRFPNITNLCPFGEVFNATRFASVYAWNRKRISNCVADYSVLYN |
| | | SASFSTFKCYGVSPTKLNDLCFTNVYADSFVIRGDEVRQIAPGQTGKIADYNYKLPDDFTGCVIAWNSNNLDSKVGGNYNY |
| | | LYRLFRKSNLKPFERDISTEIYQAGSTPCNGVEGENCYFPLQSYGFQPTNGVGYQPYRVVVLSFELLHAPATVCGPKKSTN |
| | | LVKNKCVNF |
| 110 | | MGVKVLFALICIAVAEAKKSSQYIKANSKFIGITE |
| | | RVQPTESIVRFPNITNLCPFGEVFNATRFASVYAWNRKRISNCVADYSVLYNSASFSTFKCYGVSPTKLNDLCFTNVYADSF |
| | | VIRGDEVRQIAPGQTGKIADYNYKLPDDFTGCVIAWNSNNLDSKVGGNYNYLYRLFRKSNLKPFERDISTEIYQAGSTPCNG |
| | | VEGFNCYFPLQSYGFQPTNGVGYQPYRVVVLSFELLHAPATVCGPKKSTNLVKNKCVNF |
| 111 | 2A7520 | MGVKVLFALICIAVAEAKRVQPTESIVRFPNITNLCPFGEVFNATRFASVYAWNRKRISNCVADYSVLYNSASFSTFKCYGVS |
| | | PTKLNDLCFTNVYADSFVIRGDEVRQIAPGQTGKIADYNYKLPDDFTGCVIAWNSNNLDSKVGGNYNYLYRLFRKSNLKPFE |
| | | RDISTEIYQAGSTPCNGVEGFNCYFPLQSYGFQPTNGVGYQPYRVVVLSFELLHAPATVCGPKKSTNLVKNKCVNFGYIPE |
| | | APRDGQAYVRKDGEWVLLSTFL* |
| 112 | P46 | AUGGGCGUGCACGAGUGCCCCGCCUGGCUGUGGCUGCUGCUGAGCCUGCUGAGCCUGCCCCUGGGCCUGCCCG |
| | | UGCUGGGCGCCAGAGUCCAACCAACAGAAUCUAUUGUUAGAUUUCCUAAUAUUACAAACUUGUGCCCUUUUGGUG |
| | | AAGUUUUUAACGCCACCAGAUUUGCAUCCGUGUAUGCUUGGAACAGGAAGAGAAUCAGCAACUGUGUUGCUGAUU |
| | | AUUCUGUCCUAUAUAAUUCCGCAUCAUUUUCCACUUUUAAGUGUUAUGGAGUGUCUCCUACUAAAUUAAAUGAUC |
| | | UCUGCUUUACUAAUGUCUAUGCAGAUUCAUUUGUAAUUAGAGGUGAUGAAGUCAGACAAAUCGCUCCAGGGCAAA |
| | | CUGGAAAGAUUGCUGAUUAUAAUUAUAAAUUACCAGAUGAUUUUACAGGCUGCGUUAUAGCUUGGAAUUCUAACAA |
| | | UCUUGAUUCUAAGGUUGGUGGUAAUUAUAAUUACCUGUAUAGAUUGUUUAGGAAGUCUAAUCUCAAACCUUUUGA |
| | | GAGAGAUAUUUCAACUGAAAUCUAUCAGGCCGGUAGCACACCUUGUAAUGGUGUUGAAGGUUUUAAUUGUUACUU |
| | | UCCUUUACAAUCAUAUGGUUUCCAACCCACUAAUGGUGUUGGUUACCAACCAUACAGAGUAGUAGUACUUUCUUU |
| | | UGAACUUCUACAUGCACCAGCAACUGUUUGUGGACCUAAAAAGUCUACUAAUUUGGUUAAAAACAAAUGUGUCAAU |
| | | UUCUAA |
| 113 | P47 | AUGUACCGGAUGCAGUUGUUGUCCUGUAUAGCUCUUUCCCUUGCAUUGGUCACUAAUUCUAGAGUCCAACCAACA |
| | | GAAUCUAUUGUUAGAUUUCCUAAUAUUACAAACUUGUGCCCUUUUGGUGAAGUUUUUAACGCCACCAGAUUUGCA |
| | | UCCGUGUAUGCUUGGAACAGGAAGAGAAUCAGCAACUGUGUUGCUGAUUAUUCUGUCCUAUAUAAUUCCGCAUCA |
| | | UUUUCCACUUUUAAGUGUUAUGGAGUGUCUCCUACUAAAUUAAAUGAUCUCUGCUUUACUAAUGUCUAUGCAGAU |
| | | UCAUUUGUAAUUAGAGGUGAUGAAGUCAGACAAAUCGCUCCAGGGCAAACUGGAAAGAUUGCUGAUUAUAAUUAU |
| | | AAAUUACCAGAUGAUUUUACAGGCUGCGUUAUAGCUUGGAAUUCUAACAAUCUUGAUUCUAAGGUUGGUGGUAAU |
| | | UAUAAUUACCUGUAUAGAUUGUUUAGGAAGUCUAAUCUCAAACCUUUUGAGAGAGAUAUUUCAACUGAAAUCUAUC |
| | | AGGCCGGUAGCACACCUUGUAAUGGUGUUGAAGGUUUUAAUUGUUACUUUCCUUUACAAUCAUAUGGUUUCCAAC |
| | | CCACUAAUGGUGUUGGUUACCAACCAUACAGAGUAGUAGUACUUUCUUUUGAACUUCUACAUGCACCAGCAACUG |
| | | UUUGUGGACCUAAAAAGUCUACUAAUUUGGUUAAAAACAAAUGUGUCAAUUUCUAA |
| 114 | P48 | AUGGGAGUCAAAGUUCUGUUUGCCCUGAUCUGCAUCGCUGUGGCCGAGGCCAAGAGAGUCCAACCAACAGAAUCU |
| | | AUUGUUAGAUUUCCUAAUAUUACAAACUUGUGCCCUUUUGGUGAAGUUUUUAACGCCACCAGAUUUGCAUCCGUG |
| | | UAUGCUUGGAACAGGAAGAGAAUCAGCAACUGUGUUGCUGAUUAUUCUGUCCUAUAUAAUUCCGCAUCAUUUUCC |
| | | ACUUUUAAGUGUUAUGGAGUGUCUCCUACUAAAUUAAAUGAUCUCUGCUUUACUAAUGUCUAUGCAGAUUCAUUU |
| | | GUAAUUAGAGGUGAUGAAGUCAGACAAAUCGCUCCAGGGCAAACUGGAAAGAUUGCUGAUUAUAAUUAUAAAUUAC |
| | | CAGAUGAUUUUACAGGCUGCGUUAUAGCUUGGAAUUCUAACAAUCUUGAUUCUAAGGUUGGUGGUAAUUAUAAUU |
| | | ACCUGUAUAGAUUGUUUAGGAAGUCUAAUCUCAAACCUUUUGAGAGAGAUAUUUCAACUGAAAUCUAUCAGGCCG |
| | | GUAGCACACCUUGUAAUGGUGUUGAAGGUUUUAAUUGUUACUUUCCUUUACAAUCAUAUGGUUUCCAACCCACUA |
| | | AUGGUGUUGGUUACCAACCAUACAGAGUAGUAGUACUUUCUUUUGAACUUCUACAUGCACCAGCAACUGUUUGUG |
| | | GACCUAAAAAGUCUACUAAUUUGGUUAAAAACAAAUGUGUCAAUUUCUAA |
| 115 | P49 | AUGGGCGUGCACGAGUGCCCCGCCUGGCUGUGGCUGCUGCUGAGCCUGCUGAGCCUGCCCCUGGGCCUGCCCG |
| | | UGCUGGGCGCCAGAGUCCAACCAACAGAAUCUAUUGUUAGAUUUCCUAAUAUUACAAACUUGUGCCCUUUUGGUG |
| | | AAGUUUUUAACGCCACCAGAUUUGCAUCCGUGUAUGCUUGGAACAGGAAGAGAAUCAGCAACUGUGUUGCUGAUU |
| | | AUUCUGUCCUAUAUAAUUCCGCAUCAUUUUCCACUUUUAAGUGUUAUGGAGUGUCUCCUACUAAAUUAAAUGAUC |
| | | UCUGCUUUACUAAUGUCUAUGCAGAUUCAUUUGUAAUUAGAGGUGAUGAAGUCAGACAAAUCGCUCCAGGGCAAA |
| | | CUGGAAAGAUUGCUGAUUAUAAUUAUAAAUUACCAGAUGAUUUUACAGGCUGCGUUAUAGCUUGGAAUUCUAACAA |
| | | UCUUGAUUCUAAGGUUGGUGGUAAUUAUAAUUACCUGUAUAGAUUGUUUAGGAAGUCUAAUCUCAAACCUUUUGA |
| | | GAGAGAUAUUUCAACUGAAAUCUAUCAGGCCGGUAGCACACCUUGUAAUGGUGUUGAAGGUUUUAAUUGUUACUU |
| | | UCCUUUACAAUCAUAUGGUUUCCAACCCACUAAUGGUGUUGGUUACCAACCAUACAGAGUAGUAGUACUUUCUUU |
| | | UGAACUUCUACAUGCACCAGCAACUGUUUGUGGACCUAAAAAGUCUACUAAUUUGGUUAAAAACAAAUGUGUCAAU |
| | | UUCUAA |
| 116 | P50 | AUGUACCGGAUGCAGUUGUUGUCCUGUAUAGCUCUUUCCCUUGCAUUGGUCACUAAUUCUAGGGUUCAGCCUAC |
| | | UGAGUCCAUCGUGAGAUUCCCAAACAUCACAAAUCUUUGUCCCUUCGGUGAGGUAUUCAAUGCGACACGAUUUGC |
| | | CUCAGUGUACGCGUGGAAUCGGAAGAGGAUCUCCAAUUGCGUGGCCGACUACUCCGUCUUAUACAACUCAGCUAG |
| | | CUUUUCAACAUUCAAGUGCUAUGGCGUGAGCCCUACCAAGCUCAAUGACCUGUGCUUCACUAAUGUGUAUGCCGA |
| | | CUCUUUUGUCAUUCGCGGCGACGAGGUCCGACAAAUCGCACCGGGCCAAACCGGUAAAAUUGCCGACUACAACUA |
| | | CAAGCUGCCUGACGACUUCACCGGCUGCGUAAUCGCCUGGAACAGCAAUAACCUGGAUAGCAAAGUGGGGGAAA |
| | | CUACAACUACCUGUACCGGCUCUUUAGAAAGUCCAACCUGAAACCAUUCGAGCGCGAUAUCUCGACCGAAAUCUA |
| | | CCAGGCGGGCAGCACCCCCUGUAAUGGUGUAGAAGGGUUCAAUUGUUACUUUCCACUCCAGAGUUAUGGGUUCC |
| | | AGCCGACCAAUGGCGUCGGUUAUCAACCAUAUCGCGUUGUGGUGUUGUCCUUUGAGCUGCUACACGCCCCAGCU |
| | | ACAGUGUGCGGGCCAAAGAAAAGCACAAAUCUUGUCAAGAACAAAUGCGUUAACUUUUAA |
| 117 | P52 | AUGUACCGGAUGCAGUUGUUGUCCUGUAUAGCUCUUUCCCUUGCAUUGGUCACUAAUUCUGUGAACCUCACCACU |
| | | CGCACACAGCUACCUCCCGCGUACACUAAUUCAUUUACCAGGGGCGUCUAUUAUCCUGAUAAGGUGUUCCGGAGU |
| | | UCAGUGUUGCAUAGCACUCAAGACCUGUUCCUGCCCUUCUUCUCCAAUGUCACUUGGUUUCAUGCGAUACAUGUG |
| | | UCUGGUACCAACGGAACGAAGAGAUUUGAUAACCCCGUACUGCCAUUCAAUGAUGGCGUAUACUUUGCUUCGACU |
| | | GAAAAAUCCAACAUCAUCAGGGGCUGGAUUUUUGGUACAACGCUUGAUUCCAAGACCCAGUCCCUCCUUAUUGUG |
| | | AACAAUGCGACCAACGUCGUGAUAAAGGUCUGUGAGUUUCAGUUCUGCAAUGACCCAUUCCUUGGAGUGUAUUAU |
| | | CACAAGAACAACAAAUCGUGGAUGGAGUCAGAGUUUAGGGUGUACAGCAGCGCUAACAACUGUACAUUUGAGUAU |
| | | GUGAGUCAGCCGUUUCUGAUGGAUCUUGAGGGCAAACAGGGCAAUUUUAAGAAUCUCAGAGAAUUUGUGUUCAAG |
| | | AACAUUGAUGGUUACUUUAAGAUCUAUAGCAAACAUACGCCAAUCAACUUGGUUCGUGAUCUGCCACAGGGAUUU |
| | | AGCGCACUGGAACCUCUCGUUGACUUGCCCAUAGGUAUUAACAUCACCAGGUUCCAGACGCUCUUGGCAUUACAC |
| | | CGUAGUUAUCUGACCCCCGGGGACUCCAGUUCCGGAUGGACUGCAGGAGCCGCUGCCUACUAUGUGGGUUACCU |
| | | CCAGCCCAGGACCUUUCUUUUGAAAUAUAACGAGAACGGCACAAUCACUGAUGCUGUGGACUGCGCAUUGGAUCC |
| | | UUUGUCAGAGACUAAGUGCACUCUGAAGUCAUUCACAGUCGAGAAAGGCAUUUACCAGACGUCUAACUUCAGGGU |
| | | UCAGCCUACUGAGUCCAUCGUGAGAUUCCCAAACAUCACAAAUCUUUGUCCCUUCGGUGAGGUAUUCAAUGCGAC |
| | | ACGAUUUGCCUCAGUGUACGCGUGGAAUCGGAAGAGGAUCUCCAAUUGCGUGGCCGACUACUCCGUCUUAUACAA |
| | | CUCAGCUAGCUUUUCAACAUUCAAGUGCUAUGGCGUGAGCCCUACCAAGCUCAAUGACCUGUGCUUCACUAAUGU |
| | | GUAUGCCGACUCUUUUGUCAUUCGCGGCGACGAGGUCCGACAAAUCGCACCGGGCCAAACCGGUAAAAUUGCCG |
| | | ACUACAACUACAAGCUGCCUGACGACUUCACCGGCUGCGUAAUCGCCUGGAACAGCAAUAACCUGGAUAGCAAAG |
| | | UGGGCGGAAACUACAACUACCUGUACCGGCUCUUUAGAAAGUCCAACCUGAAACCAUUCGAGCGCGAUAUCUCGA |
| | | CCGAAAUCUACCAGGGGGGCAGCACCCCCUGUAAUGGUGUAGAAGGGUUCAAUUGUUACUUUCCACUCCAGAGUU |
| | | AUGGGUUCCAGCCGACCAAUGGCGUCGGUUAUCAACCAUAUCGCGUUGUGGUGUUGUCCUUUGAGCUGCUACAC |
| | | GCCCCAGCUACAGUGUGCGGGCCAAAGAAAAGCACAAAUCUUGUCAAGAACAAAUGCGUUAACUUUAAUUUUAAUG |
| | | GACUCACAGGUACAGGAGUCCUGACCGAAUCUAAUAAGAAGUUCCUGCCCUUUCAACAGUUCGGACGAGACAUUG |
| | | CCGACACCACCGAUGCCGUUCGGGACCCACAGACCUUAGAAAUUCUGGAUAUUACUCCAUGUAGUUUUGGGGGAG |
| | | UGUCUGUCAUCACCCCUGGCACUAAUACAUCUAACCAGGUUGCAGUCCUCUACCAGGAUGUGAACUGUACCGAAG |
| | | UGCCGGUCGCUAUUCACGCAGACCAGCUCACUCCUACCUGGGGGUGUACUCCACAGGCUCUAACGUGUUUCAG |
| | | ACACGUGCAGGGUGCCUAAUCGGCGCAGAGCAUGUAAAUAACUCCUAUGAGUGUGAUAUCCCCAUCGGAGCCGG |
| | | GAUCUGCGCUUCCUACCAGACACAAACGAAUAGUCCCGGAUCUGCCUCAAGCGUGGCAUCUCAAUCCAUUAUAGC |
| | | AUAUACGAUGUCCCUUGGAGCUGAAAACAGCGUUGCGUAUUCAAACAAUAGUAUCGCUAUUCCAACCAAUUUUACA |
| | | AUUAGCGUGACCACAGAAAUACUCCCUGUGAGCAUGACCAAGACCAGUGUAGACUGUACUAUGUACAUCUGCGGC |
| | | GACAGUACUGAGUGUAGCAAUCUGCUGCUACAGUAUGGGUCCUUCUGUACUCAGCUUAAUCGGGCUCUCACCGG |
| | | AAUCGCUGUAGAGCAAGAUAAAAACACACAAGAAGUGUUUGCUCAAGUGAAGCAGAUCUAUAAGACACCUCCCAUC |
| | | AAGGAUUUCGGUGGGUUCAACUUUAGCCAGAUUCUGCCCGAUCCGUCUAAACCGUCCAAGCGAAGUUUCAUCGAA |
| | | GACCUGCUUUUCAAUAAGGUCACGCUGGCAGAUGCUGGAUUUAUCAAACAGUACGGCGACUGUCUGGGCGAUAU |
| | | CGCCGCAAGAGACUUGAUAUGCGCCCAAAAGUUUAAUGGGUUAACCGUCCUUCCACCGCUCCUGACAGACGAGAU |
| | | GAUCGCCCAGUAUACAAGUGCCUUAUUAGCUGGGACCAUUACUAGUGGAUGGACAUUUGGCGCCGGGGCUGCUC |
| | | UACAGAUACCCUUCGCCAUGCAGAUGGCUUACCGCUUCAACGGAAUCGGAGUUACCCAGAACGUACUGUACGAAA |
| | | AUCAGAAACUCAUAGCUAAUCAAUUUAACUCUGCCAUCGGGAAGAUUCAGGAUUCCCUGUCGUCUACAGCGUCCG |
| | | CCUUGGGGAAACUGCAAGAUGUAGUGAACCAGAACGCCCAGGCCUUAAAUACUCUGGUCAAGCAGUUAUCUUCAA |
| | | AUUUCGGAGCAAUUAGCUCUGUGUUGAACGAUAUUCUUUCCAGGCUGGACCCUCCAGAAGCCGAAGUGCAAAUAG |
| | | ACCGGCUCAUCACGGGGCGCUUGCAAAGCCUGCAAACCUAUGUCACCCAGCAACUGAUUCGAGCAGCCGAGAUCC |
| | | GGGCCAGUGCUAAUCUGGCCGCCACAAAAAUGAGCGAGUGCGUCCUCGGGCAGAGCAAACGCGUAGACUUCUGC |
| | | GGUAAAGGCUAUCACCUGAUGAGCUUCCCUCAGAGCGCACCCCACGGGGUGGUCUUCCUCCACGUUACCUACGU |
| | | CCCUGCGCAGGAGAAGAACUUCACUACGGCCCCUGCAAUUUGCCACGAUGGCAAGGCCCACUUUCCCAGGGAGG |
| | | GGGUCUUCGUUUCCAACGGGACUCAUUGGUUCGUGACUCAGAGAAAUUUUUAUGAACCUCAGAUCAUUACCACUG |
| | | AUAAUACAUUCGUGUCUGGCAACUGUGAUGUGGUUAUUGGGAUAGUUAAUAAUACGGUAUACGACCCACUCCAGC |
| | | CCGAGCUGGACUCCUUCAAAGAGGAGCUGGACAAGUACUUUAAAAAUCACACCUCACCUGAUGUGGACCUAGGUG |
| | | ACAUAUCUGGCAUAAAUGCUAGCGUGGUUAACAUUCAGAAGGAAAUCGACAGACUCAACGAGGUGGCCAAAAAUC |
| | | UGAACGAGAGUCUGAUCGACCUGCAGGAGUUGGGAAAAUAUGAACAGUACAUCAAAUGGCCAUGGUACAUCUGGC |
| | | UGGGCUUCAUAGCAGGCCUGAUCGCCAUCGUCAUGGUGACUAUUAUGCUGUGCUGCAUGACAUCCUGUUGUAGC |
| | | UGUUUGAAGGGGUGUUGCUCCUGCGGCUCAUGCUGCAAAUUCGACGAGGACGAUUCAGAACCUGUGCUGAAGGG |
| | | AGUGAAGCUGCAUUACACAUAA |
| 118 | P54 | AUGGGAGUCAAAGUUCUGUUUGCCCUGAUCUGCAUCGCUGUGGCCGAGGCCAAGGUUAAUCUUACAACCAGAACU |
| | | CAAUUACCCCCUGCAUACACUAAUUCUUUCACACGUGGUGUUUAUUACCCUGACAAAGUUUUCAGAUCCUCAGUU |
| | | UUACAUUCAACUCAGGACUUGUUCUUACCUUUCUUUUCCAAUGUUACUUGGUUCCAUGCUAUACAUGUCUCUGGG |
| | | ACCAAUGGUACUAAGAGGUUUGAUAACCCUGUCCUACCAUUUAAUGAUGGUGUUUAUUUUGCUUCCACUGAGAAG |
| | | UCUAACAUAAUAAGAGGCUGGAUUUUUGGUACUACUUUAGAUUCGAAGACCCAGUCCCUACUUAUUGUUAAUAAC |
| | | GCUACUAAUGUUGUUAUUAAAGUCUGUGAAUUUCAAUUUUGUAAUGAUCCAUUUUUGGGUGUUUAUUACCACAAA |
| | | AACAACAAAAGUUGGAUGGAAAGUGAGUUCAGAGUUUAUUCUAGUGCGAAUAAUUGCACUUUUGAAUAUGUCUCU |
| | | CAGCCUUUUCUUAUGGACCUUGAAGGAAAACAGGGUAAUUUCAAAAAUCUUAGGGAAUUUGUGUUUAAGAAUAUU |
| | | GAUGGUUAUUUUAAAAUAUAUUCUAAGCACACGCCUAUUAAUUUAGUGCGUGAUCUCCCUCAGGGUUUUUCGGCU |
| | | UUAGAACCAUUGGUAGAUUUGCCAAUAGGUAUUAACAUCACUAGGUUUCAAACUUUACUUGCUUUACAUAGAAGUU |
| | | AUUUGACUCCUGGUGAUUCUUCUUCAGGUUGGACAGCUGGUGCUGCAGCUUAUUAUGUGGGUUAUCUUCAACCU |
| | | AGGACUUUUCUAUUAAAAUAUAAUGAAAAUGGAACCAUUACAGAUGCUGUAGACUGUGCACUUGACCCUCUCUCAG |
| | | AAACAAAGUGUACGUUGAAAUCCUUCACUGUAGAAAAAGGAAUCUAUCAAACUUCUAACUUUAGAGUCCAACCAAC |
| | | AGAAUCUAUUGUUAGAUUUCCUAAUAUUACAAACUUGUGCCCUUUUGGUGAAGUUUUUAACGCCACCAGAUUUGC |
| | | AUCCGUGUAUGCUUGGAACAGGAAGAGAAUCAGCAACUGUGUUGCUGAUUAUUCUGUCCUAUAUAAUUCCGCAUC |
| | | AUUUUCCACUUUUAAGUGUUAUGGAGUGUCUCCUACUAAAUUAAAUGAUCUCUGCUUUACUAAUGUCUAUGCAGA |
| | | UUCAUUUGUAAUUAGAGGUGAUGAAGUCAGACAAAUCGCUCCAGGGCAAACUGGAAAGAUUGCUGAUUAUAAUUA |
| | | UAAAUUACCAGAUGAUUUUACAGGCUGCGUUAUAGCUUGGAAUUCUAACAAUCUUGAUUCUAAGGUUGGUGGUAA |
| | | UUAUAAUUACCUGUAUAGAUUGUUUAGGAAGUCUAAUCUCAAACCUUUUGAGAGAGAUAUUUCAACUGAAAUCUAU |
| | | CAGGCCGGUAGCACACCUUGUAAUGGUGUUGAAGGUUUUAAUUGUUACUUUCCUUUACAAUCAUAUGGUUUCCAA |
| | | CCCACUAAUGGUGUUGGUUACCAACCAUACAGAGUAGUAGUACUUUCUUUUGAACUUCUACAUGCACCAGCAACU |
| | | GUUUGUGGACCUAAAAAGUCUACUAAUUUGGUUAAAAACAAAUGUGUCAAUUUCAACUUCAAUGGUUUAACAGGCA |
| | | CAGGUGUUCUUACUGAGUCUAACAAAAAGUUUCUGCCUUUCCAACAAUUUGGCAGAGACAUUGCUGACACUACUG |
| | | AUGCUGUCCGUGAUCCACAGACACUUGAGAUUCUUGACAUUACACCAUGUUCUUUUGGUGGUGUCAGUGUUAUAA |
| | | CACCAGGAACAAAUACUUCUAACCAGGUUGCUGUUCUUUAUCAGGAUGUUAACUGCACAGAAGUCCCUGUUGCUA |
| | | UUCAUGCAGAUCAACUUACUCCUACUUGGCGUGUUUAUUCUACAGGUUCUAAUGUUUUUCAAACACGUGCAGGCU |
| | | GUUUAAUAGGGGCUGAACAUGUCAACAACUCAUAUGAGUGUGACAUACCCAUUGGUGCAGGUAUAUGCGCUAGUU |
| | | AUCAGACUCAGACUAAUUCUCCUGGCAGCGCCAGCAGUGUAGCUAGUCAAUCCAUCAUUGCCUACACUAUGUCAC |
| | | UUGGUGCAGAAAAUUCAGUUGCUUACUCUAAUAACUCUAUUGCCAUACCCACAAAUUUUACUAUUAGUGUUACCAC |
| | | AGAAAUUCUACCAGUGUCUAUGACCAAGACAUCAGUAGAUUGUACAAUGUACAUUUGUGGUGAUUCAACUGAAUG |
| | | CAGCAAUCUUUUGUUGCAAUAUGGCAGUUUUUGUACACAAUUAAACCGUGCUUUAACUGGAAUAGCUGUUGAACA |
| | | AGACAAAAACACCCAAGAAGUUUUUGCACAAGUCAAACAAAUUUACAAAACACCACCAAUUAAAGAUUUUGGUGGU |
| | | UUUAAUUUUUCACAAAUAUUACCAGAUCCAUCAAAACCAAGCAAGAGGUCAUUUAUUGAAGAUCUACUUUUCAACA |
| | | AAGUGACACUUGCAGAUGCUGGCUUCAUCAAACAAUAUGGUGAUUGCCUUGGUGAUAUUGCUGCUAGGGACCUCA |
| | | UUUGUGCACAAAAGUUUAACGGCCUUACUGUUUUGCCACCUUUGCUCACAGAUGAAAUGAUUGCUCAAUACACUU |
| | | CUGCACUGUUAGCGGGUACAAUCACUUCUGGUUGGACCUUUGGUGCAGGUGCUGCAUUACAAAUACCAUUUGCU |
| | | AUGCAAAUGGCUUAUAGGUUUAAUGGUAUUGGAGUUACACAGAAUGUUCUCUAUGAGAACCAAAAAUUGAUUGCC |
| | | AACCAAUUUAAUAGUGCUAUUGGCAAAAUUCAAGACUCACUUUCUUCCACAGCAAGUGCACUUGGAAAACUUCAAG |
| | | AUGUGGUCAACCAAAAUGCACAAGCUUUAAACACGCUUGUUAAACAACUUAGCUCCAAUUUUGGUGCAAUUUCAAG |
| | | UGUUUUAAAUGAUAUCCUUUCACGUCUUGACCCUCCCGAGGCUGAAGUGCAAAUUGAUAGGUUGAUCACAGGCAG |
| | | ACUUCAAAGUUUGCAGACAUAUGUGACUCAACAAUUAAUUAGAGCUGCAGAAAUCAGAGCUUCUGCUAAUCUUGCU |
| | | GCUACUAAAAUGUCAGAGUGUGUACUUGGACAAUCAAAAAGAGUUGAUUUUUGUGGAAAGGGCUAUCAUCUUAUG |
| | | UCCUUCCCUCAGUCAGCACCUCAUGGUGUAGUCUUCUUGCAUGUGACUUAUGUCCCUGCACAAGAAAAGAACUUC |
| | | ACAACUGCUCCUGCCAUUUGUCAUGAUGGAAAAGCACACUUUCCUCGUGAAGGUGUCUUUGUUUCAAAUGGCACA |
| | | CACUGGUUUGUAACACAAAGGAAUUUUUAUGAACCACAAAUCAUUACUACAGACAACACAUUUGUGUCUGGUAACU |
| | | GUGAUGUUGUAAUAGGAAUUGUCAACAACACAGUUUAUGAUCCUUUGCAACCUGAAUUAGACUCAUUCAAGGAGG |
| | | AGUUAGAUAAAUAUUUUAAGAAUCAUACAUCACCAGAUGUUGAUUUAGGUGACAUCUCUGGCAUUAAUGCUUCAGU |
| | | UGUAAACAUUCAAAAAGAAAUUGACCGCCUCAAUGAGGUUGCCAAGAAUUUAAAUGAAUCUCUCAUCGAUCUCCAA |
| | | GAACUUGGAAAGUAUGAGCAGUAUAUAAAAUGGCCAUGGUACAUUUGGCUAGGUUUUAUAGCUGGCUUGAUUGCC |
| | | AUAGUAAUGGUGACAAUUAUGCUUUGCUGUAUGACCAGUUGCUGUAGUUGUCUCAAGGGCUGUUGUUCUUGUGG |
| | | AUCCUGCUGCAAAUUUGAUGAAGACGACUCUGAGCCAGUGCUCAAAGGAGUCAAAUUACAUUACACA |
| 119 | P56 | AUGUUUGUUUUUCUUGUUUUAUUGCCACUAGUCUCUAGUCAGUGUAGAGUCCAACCAACAGAAUCUAUUGUUAGA |
| | | UUUCCUAAUAUUACAAACUUGUGCCCUUUUGGUGAAGUUUUUAACGCCACCAGAUUUGCAUCCGUGUAUGCUUGG |
| | | AACAGGAAGAGAAUCAGCAACUGUGUUGCUGAUUAUUCUGUCCUAUAUAAUUCCGCAUCAUUUUCCACUUUUAAG |
| | | UGUUAUGGAGUGUCUCCUACUAAAUUAAAUGAUCUCUGCUUUACUAAUGUCUAUGCAGAUUCAUUUGUAAUUAGA |
| | | GGUGAUGAAGUCAGACAAAUCGCUCCAGGGCAAACUGGAAAGAUUGCUGAUUAUAAUUAUAAAUUACCAGAUGAU |
| | | UUUACAGGCUGCGUUAUAGCUUGGAAUUCUAACAAUCUUGAUUCUAAGGUUGGUGGUAAUUAUAAUUACCUGUAU |
| | | AGAUUGUUUAGGAAGUCUAAUCUCAAACCUUUUGAGAGAGAUAUUUCAACUGAAAUCUAUCAGGCCGGUAGCACA |
| | | CCUUGUAAUGGUGUUGAAGGUUUUAAUUGUUACUUUCCUUUACAAUCAUAUGGUUUCCAACCCACUAAUGGUGUU |
| | | GGUUACCAACCAUACAGAGUAGUAGUACUUUCUUUUGAACUUCUACAUGCACCAGCAACUGUUUGUGGACCUAAA |
| | | AAGUCUACUAAUUUGGUUAAAAACAAAUGUGUCAAUUUCGGGUAUAUCCCUGAAGCCCCCAGGGACGGCCAGGCU |
| | | UACGUCAGAAAGGAUGGAGAGUGGGUGCUCUUGAGCACCUUCCUGUAA |
| 120 | P59 | AUGUUUGUUUUUCUUGUUUUAUUGCCACUAGUCUCUAGUCAGUGUGUUAAUCUUACAACCAGAACUCAAUUACCC |
| | | CCUGCAUACACUAAUUCUUUCACACGUGGUGUUUAUUACCCUGACAAAGUUUUCAGAUCCUCAGUUUUACAUUCA |
| | | ACUCAGGAUUUGUUCUUACCUUUCUUUUCCAAUGUUACUUGGUUCCAUGCUAUACAUGUCUCUGGGACCAAUGGU |
| | | ACUAAGAGGUUUGAUAACCCUGUCCUACCAUUUAAUGAUGGUGUUUAUUUUGCUUCCACUGAGAAGUCUAACAUA |
| | | AUAAGAGGCUGGAUCUUUGGUACUACUUUAGAUUCGAAGACCCAGUCCCUACUUAUUGUUAAUAACGCUACUAAU |
| | | GUUGUUAUUAAAGUCUGUGAAUUUCAAUUUUGUAAUGAUCCAUUUUUGGGUGUUUAUUACCACAAAAACAACAAAA |
| | | GUUGGAUGGAAAGUGAGUUCAGAGUUUAUUCUAGUGCGAAUAAUUGCACUUUUGAAUAUGUCUCUCAGCCUUUUC |
| | | UUAUGGACCUUGAAGGAAAACAGGGUAAUUUCAAAAAUCUUAGGGAAUUUGUGUUUAAGAAUAUUGAUGGUUAUU |
| | | UUAAAAUAUAUUCUAAGCACACGCCUAUUAAUUUAGUGCGUGAUCUCCCUCAGGGUUUUUCGGCUUUAGAACCAU |
| | | UGGUAGAUUUGCCAAUAGGUAUUAACAUCACUAGGUUUCAAACUUUACUUGCUUUACAUAGAAGUUAUUUGACUC |
| | | CUGGUGAUUCUUCUUCAGGUUGGACAGCUGGUGCUGCAGCUUAUUAUGUGGGUUAUCUUCAACCUAGGACUUUU |
| | | CUAUUAAAAUAUAAUGAAAAUGGAACCAUUACAGAUGCUGUAGACUGUGCACUUGACCCUCUCUCAGAAACAAAGU |
| | | GUACGUUGAAAUCCUUCACUGUAGAAAAAGGAAUCUAUCAAACUUCUAACUUUAGAGUCCAACCAACAGAAUCUAU |
| | | UGUUAGAUUUCCUAAUAUUACAAACUUGUGCCCUUUUGGUGAAGUUUUUAACGCCACCAGAUUUGCAUCCGUGUA |
| | | UGCUUGGAACAGGAAGAGAAUCAGCAACUGUGUUGCUGAUUAUUCUGUCCUAUAUAAUUCCGCAUCAUUUUCCAC |
| | | UUUUAAGUGUUAUGGAGUGUCUCCUACUAAAUUAAAUGAUCUCUGCUUUACUAAUGUCUAUGCAGAUUCAUUUGU |
| | | AAUUAGAGGUGAUGAAGUCAGACAAAUCGCUCCAGGGCAAACUGGAAAGAUUGCUGAUUAUAAUUAUAAAUUACCA |
| | | GAUGAUUUUACAGGCUGCGUUAUAGCUUGGAAUUCUAACAAUCUUGAUUCUAAGGUUGGUGGUAAUUAUAAUUAC |
| | | CUGUAUAGAUUGUUUAGGAAGUCUAAUCUCAAACCUUUUGAGAGAGAUAUUUCAACUGAAAUCUAUCAGGCCGGU |
| | | AGCACACCUUGUAAUGGUGUUGAAGGUUUUAAUUGUUACUUUCCUUUACAAUCAUAUGGUUUCCAACCCACUAAU |
| | | GGUGUUGGUUACCAACCAUACAGAGUAGUAGUACUUUCUUUUGAACUUCUACAUGCACCAGCAACUGUUUGUGGA |
| | | CCUAAAAAGUCUACUAAUUUGGUUAAAAACAAAUGUGUCAAUUUCAACUUCAAUGGUUUAACAGGCACAGGUGUUC |
| | | UUACUGAGUCUAACAAAAAGUUUCUGCCUUUCCAACAAUUUGGCAGAGACAUUGCUGACACUACUGAUGCUGUCC |
| | | GUGAUCCACAGACACUUGAGAUUCUUGACAUUACACCAUGUUCUUUUGGUGGUGUCAGUGUUAUAACACCAGGAA |
| | | CAAAUACUUCUAACCAGGUUGCUGUUCUUUAUCAGGAUGUUAACUGCACAGAAGUCCCUGUUGCUAUUCAUGCAG |
| | | AUCAACUUACUCCUACUUGGCGUGUUUAUUCUACAGGUUCUAAUGUUUUUCAAACACGUGCAGGCUGUUUAAUAG |
| | | GGGCUGAACAUGUCAACAACUCAUAUGAGUGUGACAUACCCAUUGGUGCAGGUAUAUGCGCUAGUUAUCAGACUC |
| | | AGACUAAUUCUCCUCGGCGGGCACGUAGUGUAGCUAGUCAAUCCAUCAUUGCCUACACUAUGUCACUUGGUGCAG |
| | | AAAAUUCAGUUGCUUACUCUAAUAACUCUAUUGCCAUACCCACAAAUUUUACUAUUAGUGUUACCACAGAAAUUCU |
| | | ACCAGUGUCUAUGACCAAGACAUCAGUAGAUUGUACAAUGUACAUUUGUGGUGAUUCAACUGAAUGCAGCAAUCU |
| | | UUUGUUGCAAUAUGGCAGUUUUUGUACACAAUUAAACCGUGCUUUAACUGGGAUAGCUGUUGAACAAGACAAAAA |
| | | CACCCAAGAAGUUUUUGCACAAGUCAAACAAAUUUACAAAACACCACCAAUUAAAGAUUUUGGUGGUUUUAAUUUU |
| | | UCACAAAUAUUACCAGAUCCAUCAAAACCAAGCAAGAGGUCAUUUAUUGAAGAUCUACUUUUCAACAAAGUGACAC |
| | | UUGCAGAUGCUGGCUUCAUCAAACAAUAUGGUGAUUGCCUUGGUGAUAUUGCUGCUAGGGACCUCAUUUGUGCA |
| | | CAAAAGUUUAACGGCCUUACUGUUUUGCCACCUUUGCUCACAGAUGAAAUGAUUGCUCAAUACACUUCUGCACUG |
| | | UUAGCGGGUACAAUCACUUCUGGUUGGACCUUUGGUGCAGGUGCUGCAUUACAAAUACCAUUUGCUAUGCAAAUG |
| | | GCUUAUAGGUUUAAUGGUAUUGGAGUUACACAGAAUGUUCUCUAUGAGAACCAAAAAUUGAUUGCCAACCAAUUUA |
| | | AUAGUGCCAUUGGCAAAAUUCAAGACUCACUUUCUUCCACAGCAAGUGCACUUGGAAAACUUCAAGAUGUGGUCA |
| | | ACCAAAAUGCACAAGCUUUAAACACGCUUGUUAAACAACUUAGCUCCAAUUUUGGUGCAAUUUCAAGUGUUUUAAA |
| | | UGAUAUCCUUUCACGUCUUGACCCUCCCGAGGCUGAAGUGCAAAUUGAUAGGUUGAUCACAGGCAGACUUCAAAG |
| | | UUUGCAGACAUAUGUGACUCAACAAUUAAUUAGAGCUGCAGAAAUCAGAGCUUCUGCUAAUCUUGCUGCUACUAAA |
| | | AUGUCAGAGUGUGUACUUGGACAAUCAAAAAGAGUUGAUUUUUGUGGAAAGGGCUAUCAUCUUAUGUCCUUCCCU |
| | | CAGUCAGCACCUCAUGGUGUAGUCUUCUUGCAUGUGACUUAUGUCCCUGCACAAGAAAAGAACUUCACAACUGCU |
| | | CCUGCCAUUUGUCAUGAUGGAAAAGCACACUUUCCUCGUGAAGGUGUCUUUGUUUCAAAUGGCACACACUGGUUU |
| | | GUAACACAAAGGAAUUUUUAUGAACCACAAAUCAUUACUACAGACAACACAUUUGUGUCUGGUAACUGUGAUGUUG |
| | | UAAUAGGAAUUGUCAACAACACAGUUUAUGAUCCUUUGCAACCUGAAUUAGACUCAUUCAAGGAGGAGUUAGAUAA |
| | | AUAUUUUAAGAAUCAUACAUCACCAGAUGUUGAUUUAGGUGACAUCUCUGGCAUUAAUGCUUCAGUUGUAAACAUU |
| | | CAAAAAGAAAUUGACCGCCUCAAUGAGGUUGCCAAGAAUUUAAAUGAAUCUCUCAUCGAUCUCCAAGAACUUGGAA |
| | | AGUAUGAGCAGUAUAUAAAAUGGCCAUGGUACAUUUGGCUAGGUUUUAUAGCUGGCUUGAUUGCCAUAGUAAUGG |
| | | UGACAAUUAUGCUUUGCUGUAUGACCAGUUGCUGUAGUUGUCUCAAGGGCUGUUGUUCUUGUGGAUCCUGCUGC |
| | | AAAUUUGAUGAAGACGACUCUGAGCCAGUGCUCAAAGGAGUCAAAUUACAUUACACA |
| 121 | P60 | AUGUUUGUUUUUCUUGUUUUAUUGCCACUAGUCUCUAGUCAGUGUGUUAAUCUUACAACCAGAACUCAAUUACCC |
| | | CCUGCAUACACUAAUUCUUUCACACGUGGUGUUUAUUACCCUGACAAAGUUUUCAGAUCCUCAGUUUUACAUUCA |
| | | ACUCAGGAUUUGUUCUUACCUUUCUUUUCCAAUGUUACUUGGUUCCAUGCUAUACAUGUCUCUGGGACCAAUGGU |
| | | ACUAAGAGGUUUGAUAACCCUGUCCUACCAUUUAAUGAUGGUGUUUAUUUUGCUUCCACUGAGAAGUCUAACAUA |
| | | AUAAGAGGCUGGAUCUUUGGUACUACUUUAGAUUCGAAGACCCAGUCCCUACUUAUUGUUAAUAACGCUACUAAU |
| | | GUUGUUAUUAAAGUCUGUGAAUUUCAAUUUUGUAAUGAUCCAUUUUUGGGUGUUUAUUACCACAAAAACAACAAAA |
| | | GUUGGAUGGAAAGUGAGUUCAGAGUUUAUUCUAGUGCGAAUAAUUGCACUUUUGAAUAUGUCUCUCAGCCUUUUC |
| | | UUAUGGACCUUGAAGGAAAACAGGGUAAUUUCAAAAAUCUUAGGGAAUUUGUGUUUAAGAAUAUUGAUGGUUAUU |
| | | UUAAAAUAUAUUCUAAGCACACGCCUAUUAAUUUAGUGCGUGAUCUCCCUCAGGGUUUUUCGGCUUUAGAACCAU |
| | | UGGUAGAUUUGCCAAUAGGUAUUAACAUCACUAGGUUUCAAACUUUACUUGCUUUACAUAGAAGUUAUUUGACUC |
| | | CUGGUGAUUCUUCUUCAGGUUGGACAGCUGGUGCUGCAGCUUAUUAUGUGGGUUAUCUUCAACCUAGGACUUUU |
| | | CUAUUAAAAUAUAAUGAAAAUGGAACCAUUACAGAUGCUGUAGACUGUGCACUUGACCCUCUCUCAGAAACAAAGU |
| | | GUACGUUGAAAUCCUUCACUGUAGAAAAAGGAAUCUAUCAAACUUCUAACUUUAGAGUCCAACCAACAGAAUCUAU |
| | | UGUUAGAUUUCCUAAUAUUACAAACUUGUGCCCUUUUGGUGAAGUUUUUAACGCCACCAGAUUUGCAUCCGUGUA |
| | | UGCUUGGAACAGGAAGAGAAUCAGCAACUGUGUUGCUGAUUAUUCUGUCCUAUAUAAUUCCGCAUCAUUUUCCAC |
| | | UUUUAAGUGUUAUGGAGUGUCUCCUACUAAAUUAAAUGAUCUCUGCUUUACUAAUGUCUAUGCAGAUUCAUUUGU |
| | | AAUUAGAGGUGAUGAAGUCAGACAAAUCGCUCCAGGGCAAACUGGAAAGAUUGCUGAUUAUAAUUAUAAAUUACCA |
| | | GAUGAUUUUACAGGCUGCGUUAUAGCUUGGAAUUCUAACAAUCUUGAUUCUAAGGUUGGUGGUAAUUAUAAUUAC |
| | | CUGUAUAGAUUGUUUAGGAAGUCUAAUCUCAAACCUUUUGAGAGAGAUAUUUCAACUGAAAUCUAUCAGGCCGGU |
| | | AGCACACCUUGUAAUGGUGUUGAAGGUUUUAAUUGUUACUUUCCUUUACAAUCAUAUGGUUUCCAACCCACUAAU |
| | | GGUGUUGGUUACCAACCAUACAGAGUAGUAGUACUUUCUUUUGAACUUCUACAUGCACCAGCAACUGUUUGUGGA |
| | | CCUAAAAAGUCUACUAAUUUGGUUAAAAACAAAUGUGUCAAUUUCAACUUCAAUGGUUUAACAGGCACAGGUGUUC |
| | | UUACUGAGUCUAACAAAAAGUUUCUGCCUUUCCAACAAUUUGGCAGAGACAUUGCUGACACUACUGAUGCUGUCC |
| | | GUGAUCCACAGACACUUGAGAUUCUUGACAUUACACCAUGUUCUUUUGGUGGUGUCAGUGUUAUAACACCAGGAA |
| | | CAAAUACUUCUAACCAGGUUGCUGUUCUUUAUCAGGGUGUUAACUGCACAGAAGUCCCUGUUGCUAUUCAUGCAG |
| | | AUCAACUUACUCCUACUUGGCGUGUUUAUUCUACAGGUUCUAAUGUUUUUCAAACACGUGCAGGCUGUUUAAUAG |
| | | GGGCUGAACAUGUCAACAACUCAUAUGAGUGUGACAUACCCAUUGGUGCAGGUAUAUGCGCUAGUUAUCAGACUC |
| | | AGACUAAUUCUCCUCGGCGGGCACGUAGUGUAGCUAGUCAAUCCAUCAUUGCCUACACUAUGUCACUUGGUGCAG |
| | | AAAAUUCAGUUGCUUACUCUAAUAACUCUAUUGCCAUACCCACAAAUUUUACUAUUAGUGUUACCACAGAAAUUCU |
| | | ACCAGUGUCUAUGACCAAGACAUCAGUAGAUUGUACAAUGUACAUUUGUGGUGAUUCAACUGAAUGCAGCAAUCU |
| | | UUUGUUGCAAUAUGGCAGUUUUUGUACACAAUUAAACCGUGCUUUAACUGGGAUAGCUGUUGAACAAGACAAAAA |
| | | CACCCAAGAAGUUUUUGCACAAGUCAAACAAAUUUACAAAACACCACCAAUUAAAGAUUUUGGUGGUUUUAAUUUU |
| | | UCACAAAUAUUACCAGAUCCAUCAAAACCAAGCAAGAGGUCAUUUAUUGAAGAUCUACUUUUCAACAAAGUGACAC |
| | | UUGCAGAUGCUGGCUUCAUCAAACAAUAUGGUGAUUGCCUUGGUGAUAUUGCUGCUAGGGACCUCAUUUGUGCA |
| | | CAAAAGUUUAACGGCCUUACUGUUUUGCCACCUUUGCUCACAGAUGAAAUGAUUGCUCAAUACACUUCUGCACUG |
| | | UUAGCGGGUACAAUCACUUCUGGUUGGACCUUUGGUGCAGGUGCUGCAUUACAAAUACCAUUUGCUAUGCAAAUG |
| | | GCUUAUAGGUUUAAUGGUAUUGGAGUUACACAGAAUGUUCUCUAUGAGAACCAAAAAUUGAUUGCCAACCAAUUUA |
| | | AUAGUGCCAUUGGCAAAAUUCAAGACUCACUUUCUUCCACAGCAAGUGCACUUGGAAAACUUCAAGAUGUGGUCA |
| | | ACCAAAAUGCACAAGCUUUAAACACGCUUGUUAAACAACUUAGCUCCAAUUUUGGUGCAAUUUCAAGUGUUUUAAA |
| | | UGAUAUCCUUUCACGUCUUGACCCUCCCGAGGCUGAAGUGCAAAUUGAUAGGUUGAUCACAGGCAGACUUCAAAG |
| | | UUUGCAGACAUAUGUGACUCAACAAUUAAUUAGAGCUGCAGAAAUCAGAGCUUCUGCUAAUCUUGCUGCUACUAAA |
| | | AUGUCAGAGUGUGUACUUGGACAAUCAAAAAGAGUUGAUUUUUGUGGAAAGGGCUAUCAUCUUAUGUCCUUCCCU |
| | | CAGUCAGCACCUCAUGGUGUAGUCUUCUUGCAUGUGACUUAUGUCCCUGCACAAGAAAAGAACUUCACAACUGCU |
| | | CCUGCCAUUUGUCAUGAUGGAAAAGCACACUUUCCUCGUGAAGGUGUCUUUGUUUCAAAUGGCACACACUGGUUU |
| | | GUAACACAAAGGAAUUUUUAUGAACCACAAAUCAUUACUACAGACAACACAUUUGUGUCUGGUAACUGUGAUGUUG |
| | | UAAUAGGAAUUGUCAACAACACAGUUUAUGAUCCUUUGCAACCUGAAUUAGACUCAUUCAAGGAGGAGUUAGAUAA |
| | | AUAUUUUAAGAAUCAUACAUCACCAGAUGUUGAUUUAGGUGACAUCUCUGGCAUUAAUGCUUCAGUUGUAAACAUU |
| | | CAAAAAGAAAUUGACCGCCUCAAUGAGGUUGCCAAGAAUUUAAAUGAAUCUCUCAUCGAUCUCCAAGAACUUGGAA |
| | | AGUAUGAGCAGUAUAUAAAAUGGCCAUGGUACAUUUGGCUAGGUUUUAUAGCUGGCUUGAUUGCCAUAGUAAUGG |
| | | UGACAAUUAUGCUUUGCUGUAUGACCAGUUGCUGUAGUUGUCUCAAGGGCUGUUGUUCUUGUGGAUCCUGCUGC |
| | | AAAUUUGAUGAAGACGACUCUGAGCCAGUGCUCAAAGGAGUCAAAUUACAUUACACA |
| 122 | P6 | AUGUUUGUUUUUCUUGUUUUAUUGCCACUAGUCUCUAGUCAGUGUGUUAAUCUUACAACCAGAACUCAAUUACCC |
| | | CCUGCAUACACUAAUUCUUUCACACGUGGUGUUUAUUACCCUGACAAAGUUUUCAGAUCCUCAGUUUUACAUUCA |
| | | ACUCAGGAUUUGUUCUUACCUUUCUUUUCCAAUGUUACUUGGUUCCAUGCUAUACAUGUCUCUGGGACCAAUGGU |
| | | ACUAAGAGGUUUGAUAACCCUGUCCUACCAUUUAAUGAUGGUGUUUAUUUUGCUUCCACUGAGAAGUCUAACAUA |
| | | AUAAGAGGCUGGAUCUUUGGUACUACUUUAGAUUCGAAGACCCAGUCCCUACUUAUUGUUAAUAACGCUACUAAU |
| | | GUUGUUAUUAAAGUCUGUGAAUUUCAAUUUUGUAAUGAUCCAUUUUUGGGUGUUUAUUACCACAAAAACAACAAAA |
| | | GUUGGAUGGAAAGUGAGUUCAGAGUUUAUUCUAGUGCGAAUAAUUGCACUUUUGAAUAUGUCUCUCAGCCUUUUC |
| | | UUAUGGACCUUGAAGGAAAACAGGGUAAUUUCAAAAAUCUUAGGGAAUUUGUGUUUAAGAAUAUUGAUGGUUAUU |
| | | UUAAAAUAUAUUCUAAGCACACGCCUAUUAAUUUAGUGCGUGAUCUCCCUCAGGGUUUUUCGGCUUUAGAACCAU |
| | | UGGUAGAUUUGCCAAUAGGUAUUAACAUCACUAGGUUUCAAACUUUACUUGCUUUACAUAGAAGUUAUUUGACUC |
| | | CUGGUGAUUCUUCUUCAGGUUGGACAGCUGGUGCUGCAGCUUAUUAUGUGGGUUAUCUUCAACCUAGGACUUUU |
| | | CUAUUAAAAUAUAAUGAAAAUGGAACCAUUACAGAUGCUGUAGACUGUGCACUUGACCCUCUCUCAGAAACAAAGU |
| | | GUACGUUGAAAUCCUUCACUGUAGAAAAAGGAAUCUAUCAAACUUCUAACUUUAGAGUCCAACCAACAGAAUCUAU |
| | | UGUUAGAUUUCCUAAUAUUACAAACUUGUGCCCUUUUGGUGAAGUUUUUAACGCCACCAGAUUUGCAUCCGUGUA |
| | | UGCUUGGAACAGGAAGAGAAUCAGCAACUGUGUUGCUGAUUAUUCUGUCCUAUAUAAUUCCGCAUCAUUUUCCAC |
| | | UUUUAAGUGUUAUGGAGUGUCUCCUACUAAAUUAAAUGAUCUCUGCUUUACUAAUGUCUAUGCAGAUUCAUUUGU |
| | | AAUUAGAGGUGAUGAAGUCAGACAAAUCGCUCCAGGGCAAACUGGAAAGAUUGCUGAUUAUAAUUAUAAAUUACCA |
| | | GAUGAUUUUACAGGCUGCGUUAUAGCUUGGAAUUCUAACAAUCUUGAUUCUAAGGUUGGUGGUAAUUAUAAUUAC |
| | | CUGUAUAGAUUGUUUAGGAAGUCUAAUCUCAAACCUUUUGAGAGAGAUAUUUCAACUGAAAUCUAUCAGGCCGGU |
| | | AGCACACCUUGUAAUGGUGUUGAAGGUUUUAAUUGUUACUUUCCUUUACAAUCAUAUGGUUUCCAACCCACUAAU |
| | | GGUGUUGGUUACCAACCAUACAGAGUAGUAGUACUUUCUUUUGAACUUCUACAUGCACCAGCAACUGUUUGUGGA |
| | | CCUAAAAAGUCUACUAAUUUGGUUAAAAACAAAUGUGUCAAUUUCAACUUCAAUGGUUUAACAGGCACAGGUGUUC |
| | | UUACUGAGUCUAACAAAAAGUUUCUGCCUUUCCAACAAUUUGGCAGAGACAUUGCUGACACUACUGAUGCUGUCC |
| | | GUGAUCCACAGACACUUGAGAUUCUUGACAUUACACCAUGUUCUUUUGGUGGUGUCAGUGUUAUAACACCAGGAA |
| | | CAAAUACUUCUAACCAGGUUGCUGUUCUUUAUCAGGAUGUUAACUGCACAGAAGUCCCUGUUGCUAUUCAUGCAG |
| | | AUCAACUUACUCCUACUUGGCGUGUUUAUUCUACAGGUUCUAAUGUUUUUCAAACACGUGCAGGCUGUUUAAUAG |
| | | GGGCUGAACAUGUCAACAACUCAUAUGAGUGUGACAUACCCAUUGGUGCAGGUAUAUGCGCUAGUUAUCAGACUC |
| | | AGACUAAUUCUCCUCGGGGGCACGUAGUGUAGCUAGUCAAUCCAUCAUUGCCUACACUAUGUCACUUGGUGCAG |
| | | AAAAUUCAGUUGCUUACUCUAAUAACUCUAUUGCCAUACCCACAAAUUUUACUAUUAGUGUUACCACAGAAAUUCU |
| | | ACCAGUGUCUAUGACCAAGACAUCAGUAGAUUGUACAAUGUACAUUUGUGGUGAUUCAACUGAAUGCAGCAAUCU |
| | | UUUGUUGCAAUAUGGCAGUUUUUGUACACAAUUAAACCGUGCUUUAACUGGGAUAGCUGUUGAACAAGACAAAAA |
| | | CACCCAAGAAGUUUUUGCACAAGUCAAACAAAUUUACAAAACACCACCAAUUAAAGAUUUUGGUGGUUUUAAUUUU |
| | | UCACAAAUAUUACCAGAUCCAUCAAAACCAAGCAAGAGGUCAUUUAUUGAAGAUCUACUUUUCAACAAAGUGACAC |
| | | UUGCAGAUGCUGGCUUCAUCAAACAAUAUGGUGAUUGCCUUGGUGAUAUUGCUGCUAGGGACCUCAUUUGUGCA |
| | | CAAAAGUUUAACGGCCUUACUGUUUUGCCACCUUUGCUCACAGAUGAAAUGAUUGCUCAAUACACUUCUGCACUG |
| | | UUAGCGGGUACAAUCACUUCUGGUUGGACCUUUGGUGCAGGUGCUGCAUUACAAAUACCAUUUGCUAUGCAAAUG |
| | | GCUUAUAGGUUUAAUGGUAUUGGAGUUACACAGAAUGUUCUCUAUGAGAACCAAAAAUUGAUUGCCAACCAAUUUA |
| | | AUAGUGCCAUUGGCAAAAUUCAAGACUCACUUUCUUCCACAGCAAGUGCACUUGGAAAACUUCAAGAUGUGGUCA |
| | | ACCAAAAUGCACAAGCUUUAAACACGCUUGUUAAACAACUUAGCUCCAAUUUUGGUGCAAUUUCAAGUGUUUUAAA |
| | | UGAUAUCCUUUCACGUCUUGACCCUCCCGAGGCUGAAGUGCAAAUUGAUAGGUUGAUCACAGGCAGACUUCAAAG |
| | | UUUGCAGACAUAUGUGACUCAACAAUUAAUUAGAGCUGCAGAAAUCAGAGCUUCUGCUAAUCUUGCUGCUACUAAA |
| | | AUGUCAGAGUGUGUACUUGGACAAUCAAAAAGAGUUGAUUUUUGUGGAAAGGGCUAUCAUCUUAUGUCCUUCCCU |
| | | CAGUCAGCACCUCAUGGUGUAGUCUUCUUGCAUGUGACUUAUGUCCCUGCACAAGAAAAGAACUUCACAACUGCU |
| | | CCUGCCAUUUGUCAUGAUGGAAAAGCACACUUUCCUCGUGAAGGUGUCUUUGUUUCAAAUGGCACACACUGGUUU |
| | | GUAACACAAAGGAAUUUUUAUGAACCACAAAUCAUUACUACAGACAACACAUUUGUGUCUGGUAACUGUGAUGUUG |
| | | UAAUAGGAAUUGUCAACAACACAGUUUAUGAUCCUUUGCAACCUGAAUUAGACUCAUUCAAGGAGGAGUUAGAUAA |
| | | AUAUUUUAAGAAUCAUACAUCACCAGAUGUUGAUUUAGGUGACAUCUCUGGCAUUAAUGCUUCAGUUGUAAACAUU |
| | | CAAAAAGAAAUUGACCGCCUCAAUGAGGUUGCCAAGAAUUUAAAUGAAUCUCUCAUCGAUCUCCAAGAACUUGGAA |
| | | AGUAUGAGCAGUAUAUAAAAUGGCCAUGGUACAUUUGGCUAGGUUUUAUAGCUGGCUUGAUUGCCAUAGUAAUGG |
| | | UGACAAUUAUGCUUUGCUGUAUGACCAGUUGCUGUAGUUGUCUCAAGGGCUGUUGUUCUUGUGGAUCCUGCUGC |
| | | AAAUUUGAUGAAGACGACUCUGAGCCAGUGCUCAAAGGAGUCAAAUUACAUUACACA |
| 123 | P63 | AUGGGCGUGCACGAGUGCCCCGCCUGGCUGUGGCUGCUGCUGAGCCUGCUGAGCCUGCCCCUGGGCCUGCCCG |
| | | UGCUGGGCGCCAGAGUCCAACCAACAGAAUCUAUUGUUAGAUUUCCUAAUAUUACAAACUUGUGCCCUUUUGGUG |
| | | AAGUUUUUAACGCCACCAGAUUUGCAUCCGUGUAUGCUUGGAACAGGAAGAGAAUCAGCAACUGUGUUGCUGAUU |
| | | AUUCUGUCCUAUAUAAUUCCGCAUCAUUUUCCACUUUUAAGUGUUAUGGAGUGUCUCCUACUAAAUUAAAUGAUC |
| | | UCUGCUUUACUAAUGUCUAUGCAGAUUCAUUUGUAAUUAGAGGUGAUGAAGUCAGACAAAUCGCUCCAGGGCAAA |
| | | CUGGAAAGAUUGCUGAUUAUAAUUAUAAAUUACCAGAUGAUUUUACAGGCUGCGUUAUAGCUUGGAAUUCUAACAA |
| | | UCUUGAUUCUAAGGUUGGUGGUAAUUAUAAUUACCUGUAUAGAUUGUUUAGGAAGUCUAAUCUCAAACCUUUUGA |
| | | GAGAGAUAUUUCAACUGAAAUCUAUCAGGCCGGUAGCACACCUUGUAAUGGUGUUGAAGGUUUUAAUUGUUACUU |
| | | UCCUUUACAAUCAUAUGGUUUCCAACCCACUAAUGGUGUUGGUUACCAACCAUACAGAGUAGUAGUACUUUCUUU |
| | | UGAACUUCUACAUGCACCAGCAACUGUUUGUGGACCUAAAAAGUCUACUAAUUUGGUUAAAAACAAAUGUGUCAAU |
| | | UUCUAA |
| 124 | P64 | AUGUACCGGAUGCAGUUGUUGUCCUGUAUAGCUCUUUCCCUUGCAUUGGUCACUAAUUCUAGAGUCCAACCAACA |
| | | GAAUCUAUUGUUAGAUUUCCUAAUAUUACAAACUUGUGCCCUUUUGGUGAAGUUUUUAACGCCACCAGAUUUGCA |
| | | UCCGUGUAUGCUUGGAACAGGAAGAGAAUCAGCAACUGUGUUGCUGAUUAUUCUGUCCUAUAUAAUUCCGCAUCA |
| | | UUUUCCACUUUUAAGUGUUAUGGAGUGUCUCCUACUAAAUUAAAUGAUCUCUGCUUUACUAAUGUCUAUGCAGAU |
| | | UCAUUUGUAAUUAGAGGUGAUGAAGUCAGACAAAUCGCUCCAGGGCAAACUGGAAAGAUUGCUGAUUAUAAUUAU |
| | | AAAUUACCAGAUGAUUUUACAGGCUGCGUUAUAGCUUGGAAUUCUAACAAUCUUGAUUCUAAGGUUGGUGGUAAU |
| | | UAUAAUUACCUGUAUAGAUUGUUUAGGAAGUCUAAUCUCAAACCUUUUGAGAGAGAUAUUUCAACUGAAAUCUAUC |
| | | AGGCCGGUAGCACACCUUGUAAUGGUGUUGAAGGUUUUAAUUGUUACUUUCCUUUACAAUCAUAUGGUUUCCAAC |
| | | CCACUAAUGGUGUUGGUUACCAACCAUACAGAGUAGUAGUACUUUCUUUUGAACUUCUACAUGCACCAGCAACUG |
| | | UUUGUGGACCUAAAAAGUCUACUAAUUUGGUUAAAAACAAAUGUGUCAAUUUCUAA |
| 125 | 2A6200 | AUGUUCGUGUUCCUGGUGCUGCUCCCCCUGGUGAGCUCCCAGUGCGUCAACUUUACCAACCGGACCCAGCUGCC |
| | | CAGUGCCUAUACCAACAGCUUCACCCGCGGCGUCUACUACCCCGACAAGGUGUUCCGGAGCAGCGUGCUGCACA |
| | | GCACCCAGGACCUCUUCCUCCCCUUUUUUAGCAACGUGACCUGGUUCCACGCCAUCCACGUGAGUGGCACCAACG |
| | | GCACCAAGCGGUUCGACAACCCUGUGCUCCCCUUCAACGACGGCGUGUACUUCGCUAGCACAGAAAAAAGUAACA |
| | | UCAUUCGGGGCUGGAUCUUCGGCACCACACUCGACAGUAAGACCCAGAGCCUGCUGAUUGUGAAUAACGCCACCA |
| | | ACGUGGUGAUCAAGGUGUGCGAGUUCCAGUUCUGCAACUACCCCUUCCUGGGCGUGUACUACCACAAGAACAACA |
| | | AGAGCUGGAUGGAGAGCGAGUUCCGCGUGUAUAGCUCUGCCAACAACUGCACCUUUGAGUACGUGAGCCAGCCC |
| | | UUCCUGAUGGACCUGGAGGGCAAGCAGGGCAAUUUCAAAAACCUCAGCGAGUUCGUGUUCAAGAACAUCGACGGC |
| | | UACUUCAAGAUCUACAGCAAGCACACCCCCAUCAACCUGGUGCGGGACCUGCCCCAGGGCUUUAGCGCUCUGGAG |
| | | CCCCUGGUGGACCUGCCCAUCGGCAUCAACAUCACCCGCUUCCAAACCCUGUUGGCCCUGCACCGGAGCUACCU |
| | | GACCCCUGGCGAUAGCAGUAGUGGCUGGACAGCCGGAGCCGCCGCCUACUACGUGGGAUACCUGCAGCCCCGGA |
| | | CCUUCCUGCUGAAGUACAACGAGAACGGCACAAUCACAGACGCUGUGGACUGCGCCCUGGACCCCCUGAGCGAAA |
| | | CAAAGUGCACCCUCAAAAGUUUCACCGUGGAGAAGGGCAUCUACCAGACCAGCAACUUCCGGGUGCAGCCCACCG |
| | | AGAGCAUCGUGCGGUUCCCCAACAUCACCAACCUGUGCCCCUUCGGCGAGGUGUUCAACGCCACCCGGUUCGCC |
| | | AGCGUGUACGCCUGGAACCGGAAGCGGAUCAGCAACUGCGUGGCCGACUACAGCGUGCUGUAUAACAGUGCCAG |
| | | CUUCAGCACCUUCAAGUGCUACGGCGUGAGCCCCACCAAGCUGAACGACCUGUGCUUCACCAACGUGUACGCCGA |
| | | UAGCUUUGUGAUCCGGGGCGACGAGGUGCGGCAGAUCGCCCCCGGCCAGACAGGCACAAUUGCCGAUUACAAUU |
| | | ACAAGCUGCCCGACGAUUUCACCGGCUGCGUGAUCGCCUGGAACAGCAACAACCUGGACAGCAAGGUGGGGGA |
| | | AACUACAACUACCUGUACCGGCUGUUCCGCAAAAGUAACCUCAAGCCUUUCGAGCGGGACAUCAGCACCGAGAUC |
| | | UACCAGGCCGGCAGUACCCCUUGCAACGGCGUGAAGGGCUUCAACUGCUACUUCCCCCUGCAGAGCUACGGCUU |
| | | CCAGCCCACCUACGGCGUGGGCUACCAGCCCUACCGGGUGGUGGUUCUGAGCUUCGAGCUGCUGCACGCCCCUG |
| | | CCACCGUGUGCGGCCCCAAGAAAAGCACCAACCUGGUGAAGAACAAGUGCGUGAACUUCAACUUUAACGGACUGA |
| | | CAGGCACCGGCGUGCUGACCGAGAGCAACAAGAAGUUCCUGCCCUUCCAGCAAUUCGGCCGGGAUAUUGCCGAC |
| | | ACCACCGACGCCGUGCGGGACCCCCAGACCCUGGAGAUCCUGGACAUCACACCCUGUAGCUUCGGCGGCGUGAG |
| | | CGUGAUCACCCCCGGCACCAACACCAGCAACCAGGUGGCCGUGCUCUACCAAGGCGUGAACUGCACCGAGGUGC |
| | | CCGUGGCCAUCCACGCCGACCAGCUGACCCCCACCUGGCGGGUGUACAGCACCGGCAGCAACGUGUUCCAGACC |
| | | CGGGCCGGCUGCCUGAUCGGCGCCGAGUACGUGAAUAACAGCUACGAGUGCGACAUCCCCAUCGGAGCCGGAAU |
| | | CUGCGCCAGCUACCAGACCCAGACCAACAGCCCCCGGAGAGCCCGGAGCGUGGCCAGCCAGAGCAUUAUUGCCU |
| | | ACACCAUGAGCCUGGGCGCCGAGAACAGCGUGGCCUACAGCAACAAUAGUAUUGCCAUCCCCACCAACUUCACCA |
| | | UCAGCGUGACCACUGAAAUCCUCCCCGUGAGCAUGACCAAGACCAGCGUGGACUGCACCAUGUACAUCUGCGGCG |
| | | ACAGCACCGAGUGCAGCAACCUGCUCCUGCAGUACGGCAGCUUCUGCACCCAGCUGAACCGGGCCCUGACCGGA |
| | | AUUGCCGUGGAGCAGGACAAGAACACCCAGGAGGUGUUCGCCCAGGUGAAGCAGAUCUACAAGACACCUCCUAUC |
| | | AAGGACUUCGGCGGGUUCAACUUCAGCCAGAUCCUGCCCGACCCCAGCAAGCCCAGCAAGCGGAGCUUCAUCGA |
| | | GGACCUGUUGUUCAACAAGGUGACCCUGGCCGACGCCGGCUUCAUCAAGCAGUACGGCGACUGUCUGGGAGAUA |
| | | UUGCCGCACGGGACCUGAUCUGCGCCCAGAAGUUCAACGGCCUGACCGUGCUGCCUCCUCUGCUGACCGACGAG |
| | | AUGAUCGCCCAGUACACAAGCGCUCUGCUCGCCGGCACCAUCACCAGCGGCUGGACCUUCGGCGCCGGCGCUGC |
| | | CCUGCAGAUCCCCUUCGCCAUGCAGAUGGCCUACCGGUUCAACGGCAUCGGCGUGACCCAGAACGUGCUGUACG |
| | | AGAACCAGAAGCUGAUCGCCAACCAGUUCAACAGCGCCAUCGGCAAGAUCCAGGACAGCCUGAGCUCCACCGCCA |
| | | GCGCCCUGGGCAAGCUGCAGGACGUGGUGAACCAGAACGCCCAGGCCCUGAACACCCUGGUGAAGCAGCUGAGC |
| | | AGCAACUUCGGCGCCAUCAGCUCUGUGCUGAACGACAUCCUGAGCCGGCUGGACCCCCCAGAGGCCGAGGUGCA |
| | | AAUCGAUCGGCUCAUCACCGGCCGGCUGCAGAGCCUGCAGACCUACGUGACACAGCAGCUGAUCCGGGCCGCCG |
| | | AGAUCCGGGCCAGCGCCAACCUGGCCGCUAUCAAGAUGAGCGAGUGCGUGCUGGGCCAGAGCAAGCGGGUGGAC |
| | | UUCUGCGGCAAGGGCUACCACCUGAUGAGCUUCCCCCAGAGCGCCCCCCACGGCGUGGUGUUCCUGCACGUGAC |
| | | CUACGUGCCCGCCCAGGAGAAGAACUUCACCACCGCCCCCGCCAUCUGCCACGACGGCAAGGCCCACUUCCCCCG |
| | | GGAGGGCGUGUUCGUGAGCAACGGCACCCACUGGUUCGUGACCCAGCGGAACUUCUACGAGCCCCAGAUCAUAA |
| | | CCACCGACAACACCUUCGUGAGCGGCAACUGCGACGUGGUCAUCGGCAUCGUGAACAACACCGUGUACGACCCCC |
| | | UGCAGCCCGAGCUGGACAGCUUCAAGGAGGAACUGGACAAGUACUUCAAGAACCACACCAGCCCCGACGUGGACC |
| | | UGGGCGACAUCAGCGGCAUCAACGCCAGCUUCGUGAACAUCCAGAAGGAGAUCGACCGGCUGAACGAGGUGGCC |
| | | AAGAACCUGAACGAGAGCCUGAUCGACCUGCAGGAGCUGGGCAAGUACGAGCAGUACAUCAAGUGGCCCUGGUAC |
| | | AUCUGGCUGGGCUUUAUUGCCGGCCUGAUCGCCAUCGUGAUGGUGACCAUCAUGCUGUGCUGUAUGACAAGCUG |
| | | UUGCAGUUGCCUGAAGGGCUGCUGCAGCUGCGGCAGCUGCUGCAAGUUCGACGAGGACGACAGCGAGCCCGUGC |
| | | UGAAGGGCGUGAAGCUGCACUACACC |
| 126 | 2A0620 | AUGUUCGUGUUCCUGGUGCUGCUCCCCCUGGUGAGCUCCCAGUGCGUCAACCUGACCACACGGACCCAGCUGCC |
| | | CCCCGCCUAUACCAACAGCUUCACCCGCGGCGUCUACUACCCCGACAAGGUGUUCCGGAGCAGCGUGCUGCACA |
| | | GCACCCAGGACCUCUUCCUCCCCUUUUUUAGCAACGUGACCUGGUUCCACGCCAUCCACGUGAGUGGCACCAACG |
| | | GCACCAAGCGGUUCGCCAACCCUGUGCUCCCCUUCAACGACGGCGUGUACUUCGCUAGCACAGAAAAAAGUAACA |
| | | UCAUUCGGGGCUGGAUCUUCGGCACCACACUGGAUAGCAAAACCCAGAGCCUGCUGAUUGUGAAUAACGCCACCA |
| | | ACGUGGUGAUCAAGGUGUGCGAGUUCCAGUUCUGCAACGACCCCUUCCUGGGCGUGUACUACCACAAGAACAACA |
| | | AGAGCUGGAUGGAGAGCGAGUUCCGCGUGUAUAGCUCUGCCAACAACUGCACCUUCGAGUACGUGAGCCAGCCC |
| | | UUCCUGAUGGACCUGGAGGGCAAGCAGGGCAAUUUCAAAAACCUGCGGGAGUUCGUGUUCAAGAACAUCGACGG |
| | | CUACUUCAAGAUCUACAGCAAGCACACCCCCAUCAACCUGGUGCGGGGCCUGCCCCAGGGCUUUAGCGCUCUGG |
| | | AGCCCCUGGUGGACCUGCCCAUCGGCAUCAACAUCACCCGCUUCCAAACCCUGUUGGCCCUGCACCGGAGCUAC |
| | | CUGACCCCUGGCGAUAGCAGUAGUGGCUGGACAGCCGGAGCCGCCGCCUACUACGUGGGAUACCUGCAGCCCCG |
| | | GACCUUCCUGCUGAAGUACAACGAGAACGGCACAAUCACAGACGCCGUGGACUGCGCCCUGGACCCCCUGAGCGA |
| | | AACAAAGUGCACCCUCAAAAGUUUCACCGUGGAGAAGGGCAUCUACCAGACCAGCAACUUCCGGGUGCAGCCCAC |
| | | CGAGAGCAUCGUGCGGUUCCCCAACAUCACCAACCUGUGCCCCUUCGGCGAGGUGUUCAACGCCACCCGGUUCG |
| | | CCAGCGUGUACGCCUGGAACCGGAAGCGGAUCAGCAACUGCGUGGCCGACUACAGCGUGCUGUAUAACAGUGCC |
| | | AGCUUCAGCACCUUCAAGUGCUACGGCGUGAGCCCCACCAAGCUGAACGACCUGUGCUUCACCAACGUGUACGCC |
| | | GACAGCUUCGUGAUCCGGGGCGACGAGGUGCGGCAGAUCGCCCCCGGCCAGACCGGCAAUAUUGCUGACUAUAA |
| | | CUAUAAGCUGCCCGACGAUUUCACCGGCUGCGUGAUCGCCUGGAACAGCAACAACCUGGACAGCAAGGUGGGCG |
| | | GAAACUACAACUACCUGUACCGGCUGUUCCGCAAAAGUAACCUCAAGCCUUUCGAGCGGGACAUCAGCACCGAGA |
| | | UCUACCAGGCCGGCAGUACCCCUUGCAACGGCGUGAAGGGCUUCAACUGCUACUUCCCCCUGCAGAGCUACGGC |
| | | UUCCAGCCCACCUACGGCGUGGGCUACCAGCCCUACCGGGUGGUGGUUCUGAGCUUCGAGCUGCUGCACGCCCC |
| | | UGCCACCGUGUGCGGCCCCAAGAAAAGCACCAACCUGGUGAAGAACAAGUGCGUGAACUUCAACUUUAACGGACU |
| | | GACAGGCACCGGCGUGCUGACCGAGAGCAACAAGAAGUUCCUGCCCUUCCAGCAAUUCGGCCGGGAUAUUGCCG |
| | | ACACCACUGACGCCGUGCGGGACCCCCAGACCCUGGAGAUCCUGGACAUCACACCCUGUAGCUUCGGCGGCGUG |
| | | AGCGUGAUCACCCCCGGCACCAACACCAGCAACCAGGUGGCCGUGCUCUACCAAGGCGUGAACUGCACCGAGGU |
| | | GCCCGUGGCCAUCCACGCCGACCAGCUGACCCCCACCUGGGGGGUGUACAGCACCGGCAGCAACGUGUUCCAGA |
| | | CCCGGGCCGGCUGCCUGAUCGGCGCCGAGCACGUGAAUAACAGCUACGAGUGCGACAUCCCCAUCGGAGCCGGA |
| | | AUCUGCGCCAGCUACCAGACCCAGACCAACAGCCCCCGGAGAGCCCGGAGCGUGGCCAGCCAGAGCAUUAUUGC |
| | | CUACACCAUGAGCCUGGGCGUGGAGAACAGCGUGGCCUACAGCAACAAUAGUAUUGCCAUCCCCACCAACUUCAC |
| | | CAUCAGCGUGACCACCGAAAUCCUCCCCGUGAGCAUGACCAAGACCAGCGUGGACUGCACCAUGUACAUCUGCGG |
| | | CGACAGCACCGAGUGCAGCAACCUGCUCCUGCAGUACGGCAGCUUCUGCACCCAGCUGAACCGGGCCCUGACCG |
| | | GAAUUGCCGUGGAGCAGGACAAGAACACCCAGGAGGUGUUCGCCCAGGUGAAGCAGAUCUACAAGACACCUCCCA |
| | | UCAAGGACUUCGGCGGGUUCAACUUCAGCCAGAUCCUGCCCGACCCCAGCAAGCCCAGCAAGCGGAGCUUCAUC |
| | | GAGGACCUGUUGUUCAACAAGGUGACCCUGGCCGACGCCGGCUUCAUCAAGCAGUACGGCGACUGUCUGGGAGA |
| | | UAUUGCCGCACGGGACCUGAUCUGCGCCCAGAAGUUCAACGGCCUGACCGUGCUGCCCCCACUGCUGACCGACG |
| | | AGAUGAUCGCCCAGUACACAAGCGCUCUGCUCGCCGGCACCAUCACCAGCGGCUGGACCUUCGGCGCCGGCGCU |
| | | GCCCUGCAGAUCCCCUUCGCCAUGCAGAUGGCCUACCGGUUCAACGGCAUCGGCGUGACCCAGAACGUGCUGUA |
| | | CGAGAACCAGAAGCUGAUCGCCAACCAGUUCAACAGCGCCAUCGGCAAGAUCCAGGACAGCCUGAGCUCCACCGC |
| | | CAGCGCCCUGGGCAAGCUGCAGGACGUGGUGAACCAGAACGCCCAGGCCCUGAACACCCUGGUGAAGCAGCUGA |
| | | GCAGCAACUUCGGCGCCAUCAGCUCUGUGCUGAACGACAUCCUGAGCCGGCUGGAUCCUCCUGAGGCCGAGGUG |
| | | CAAAUCGAUCGGCUCAUCACCGGCCGGCUGCAGAGCCUGCAGACCUACGUGACACAGCAGCUGAUCCGGGCCGC |
| | | CGAGAUCCGGGCCAGCGCCAACCUGGCCGCUACCAAGAUGAGCGAGUGCGUGCUGGGCCAGAGCAAGCGGGUGG |
| | | ACUUCUGCGGCAAGGGCUACCACCUGAUGAGCUUCCCCCAGAGCGCCCCCCACGGCGUGGUGUUCCUGCACGUG |
| | | ACCUACGUGCCCGCCCAGGAGAAGAACUUCACCACCGCCCCCGCCAUCUGCCACGACGGCAAGGCCCACUUCCCC |
| | | CGGGAGGGCGUGUUCGUGAGCAACGGCACCCACUGGUUCGUGACCCAGCGGAACUUCUACGAGCCCCAGAUCAU |
| | | AACCACAGACAACACCUUCGUGAGCGGCAACUGCGACGUGGUCAUCGGCAUCGUGAACAACACCGUGUACGACCC |
| | | CCUGCAGCCCGAGCUGGACAGCUUCAAGGAGGAACUGGACAAGUACUUCAAGAACCACACCAGCCCCGACGUGGA |
| | | CCUGGGCGACAUCAGCGGCAUCAACGCCAGCGUGGUGAACAUCCAGAAGGAGAUCGACCGGCUGAACGAGGUGG |
| | | CCAAGAACCUGAACGAGAGCCUGAUCGACCUGCAGGAGCUGGGCAAGUACGAGCAGUACAUCAAGUGGCCCUGG |
| | | UACAUCUGGCUGGGCUUUAUUGCCGGCCUGAUCGCCAUCGUGAUGGUGACCAUCAUGCUGUGCUGUAUGACAAG |
| | | CUGUUGCAGUUGCCUGAAGGGCUGCUGCAGCUGCGGCAGCUGCUGCAAGUUCGACGAGGACGACAGCGAGCCCG |
| | | UGCUGAAGGGCGUGAAGCUGCACUACACC |
| 127 | 2A3430 | AUGUUCGUGUUCCUGGUGCUGCUCCCCCUGGUGAGCUCCCAGUGCGUCAACCUGACCACACGGACCCAGCUGCC |
| | | CCCCGCCUAUACCAACAGCUUCACCCGGGGCGUGUACUAUCCCGACAAGGUGUUCCGGAGCAGCGUGCUGCACA |
| | | GCACCCAGGACCUCUUCCUCCCCUUUUUUAGCAACGUGACCUGGUUCCACGCUAUCAGUGGCACCAACGGCACCA |
| | | AGCGGUUCGACAACCCUGUGCUCCCCUUCAACGACGGCGUGUACUUCGCUAGCACAGAAAAAAGUAACAUCAUUC |
| | | GGGGCUGGAUCUUCGGCACCACACUGGAUAGCAAAACCCAGAGCCUGCUGAUUGUGAAUAACGCCACCAACGUGG |
| | | UGAUCAAGGUGUGCGAGUUCCAGUUCUGCAACGACCCCUUCCUGGGCGUGUACCACAAGAACAACAAGAGCUGGA |
| | | UGGAGAGCGAGUUCCGCGUGUAUAGCUCUGCCAACAACUGCACCUUCGAGUACGUGAGCCAGCCCUUCCUGAUG |
| | | GACCUGGAGGGCAAGCAGGGCAAUUUCAAAAACCUGCGGGAGUUCGUGUUCAAGAACAUCGACGGCUACUUCAAG |
| | | AUCUACAGCAAGCACACCCCCAUCAACCUGGUGCGGGACCUGCCCCAGGGCUUUAGCGCUCUGGAGCCCCUGGU |
| | | GGACCUGCCCAUCGGCAUCAACAUCACCCGCUUCCAAACCCUGUUGGCCCUGCACCGGAGCUACCUGACCCCUG |
| | | GCGAUAGCAGUAGUGGCUGGACAGCCGGAGCCGCCGCCUACUACGUGGGAUACCUGCAGCCCCGGACCUUCCUG |
| | | CUGAAGUACAACGAGAACGGCACAAUCACAGACGCCGUGGACUGCGCCCUGGACCCCCUGAGCGAAACAAAGUGC |
| | | ACCCUCAAAAGUUUCACCGUGGAGAAGGGCAUCUACCAGACCAGCAACUUCCGGGUGCAGCCCACCGAGAGCAUC |
| | | GUGCGGUUCCCCAACAUCACCAACCUGUGCCCCUUCGGCGAGGUGUUCAACGCCACCCGGUUCGCCAGCGUGUA |
| | | CGCCUGGAACCGGAAGCGGAUCAGCAACUGCGUGGCCGACUACAGCGUGCUGUAUAACAGUGCCAGCUUCAGCA |
| | | CCUUCAAGUGCUACGGCGUGAGCCCCACCAAGCUGAACGACCUGUGCUUCACCAACGUGUACGCCGACAGCUUC |
| | | GUGAUCCGGGGCGACGAGGUGCGGCAGAUCGCCCCCGGCCAGACCGGCAAGAUCGCCGAUUACAAUUACAAGCU |
| | | GCCCGACGAUUUCACCGGCUGCGUGAUCGCCUGGAACAGCAACAACCUGGACAGCAAGGUGGGCGGAAACUACAA |
| | | CUACCUGUACCGGCUGUUCCGCAAAAGUAACCUGAAGCCCUUCGAGCGGGACAUCAGCACCGAGAUCUACCAGGC |
| | | CGGCAGUACCCCUUGCAACGGCGUGGAGGGCUUCAACUGCUACUUCCCCCUGCAGAGCUACGGCUUCCAGCCCA |
| | | CCUACGGCGUGGGCUACCAGCCCUACCGGGUGGUGGUUCUGAGCUUCGAGCUGCUGCACGCCCCUGCCACCGU |
| | | GUGCGGCCCCAAGAAAAGCACCAACCUGGUGAAGAACAAGUGCGUGAACUUCAACUUUAACGGACUGACAGGCAC |
| | | CGGCGUGCUGACCGAGAGCAACAAGAAGUUCCUGCCCUUCCAGCAAUUCGGCCGGGACAUCGACGACACCACUGA |
| | | CGCCGUGCGGGACCCCCAGACCCUGGAGAUCCUGGACAUCACACCCUGUAGCUUCGGCGGCGUGAGCGUGAUCA |
| | | CCCCCGGCACCAACACCAGCAACCAGGUGGCCGUGCUCUACCAAGGCGUGAACUGCACCGAGGUGCCCGUGGCC |
| | | AUCCACGCCGACCAGCUGACCCCCACCUGGGGGGUGUACAGCACCGGCAGCAACGUGUUCCAGACCCGGGCCGG |
| | | CUGCCUGAUCGGCGCCGAGCACGUGAAUAACAGCUACGAGUGCGACAUCCCCAUCGGAGCCGGAAUCUGCGCCA |
| | | GCUACCAGACCCAGACCAACAGCCACCGGAGAGCCCGGAGCGUGGCCAGCCAGAGCAUUAUUGCCUACACCAUGA |
| | | GCCUGGGCGCCGAGAACAGCGUGGCCUACAGCAACAAUAGUAUUGCCAUCCCCACCAACUUCACCAUCAGCGUGA |
| | | CCACCGAAAUCCUCCCCGUGAGCAUGACCAAGACCAGCGUGGACUGCACCAUGUACAUCUGCGGCGACAGCACCG |
| | | AGUGCAGCAACCUGCUCCUGCAGUACGGCAGCUUCUGCAUCCAGCUGAACCGGGCCCUGACCGGAAUUGCCGUG |
| | | GAGCAGGACAAGAACACCCAGGAGGUGUUCGCCCAGGUGAAGCAGAUCUACAAGACACCUCCCAUCAAGGACUUC |
| | | GGCGGGUUCAACUUCAGCCAGAUCCUGCCCGACCCCAGCAAGCCCAGCAAGCGGAGCUUCAUCGAGGACCUGUU |
| | | GUUCAACAAGGUGACCCUGGCCGACGCCGGCUUCAUCAAGCAGUACGGCGACUGUCUGGGAGAUAUUGCCGCAC |
| | | GGGACCUGAUCUGCGCCCAGAAGUUCAACGGCCUGACCGUGCUGCCCCCACUGCUGACCGACGAGAUGAUCGCC |
| | | CAGUACACAAGCGCUCUGCUCGCCGGCACCAUCACCAGCGGCUGGACCUUCGGCGCCGGCGCUGCCCUGCAGAU |
| | | CCCCUUCGCCAUGCAGAUGGCCUACCGGUUCAACGGCAUCGGCGUGACCCAGAACGUGCUGUACGAGAACCAGA |
| | | AGCUGAUCGCCAACCAGUUCAACAGCGCCAUCGGCAAGAUCCAGGACAGCCUGAGCUCCACCGCCAGCGCCCUGG |
| | | GCAAGCUGCAGGACGUGGUGAACCAGAACGCCCAGGCCCUGAACACCCUGGUGAAGCAGCUGAGCAGCAACUUC |
| | | GGCGCCAUCAGCUCUGUGCUGAACGACAUCCUGGCCCGGCUGGAUCCUCCUGAGGCCGAGGUGCAAAUCGAUCG |
| | | GCUCAUCACCGGCCGGCUGCAGAGCCUGCAGACCUACGUGACACAGCAGCUGAUCCGGGCCGCCGAGAUCCGGG |
| | | CCAGCGCCAACCUGGCCGCUACCAAGAUGAGCGAGUGCGUGCUGGGCCAGAGCAAGCGGGUGGACUUCUGCGGC |
| | | AAGGGCUACCACCUGAUGAGCUUCCCCCAGAGCGCCCCCCACGGCGUGGUGUUCCUGCACGUGACCUACGUGCC |
| | | CGCCCAGGAGAAGAACUUCACCACCGCCCCCGCCAUCUGCCACGACGGCAAGGCCCACUUCCCCCGGGAGGGCG |
| | | UGUUCGUGAGCAACGGCACCCACUGGUUCGUGACCCAGCGGAACUUCUACGAGCCCCAGAUCAUAACCACACACA |
| | | ACACCUUCGUGAGCGGCAACUGCGACGUGGUCAUCGGCAUCGUGAACAACACCGUGUACGACCCCCUGCAGCCC |
| | | GAGCUGGACAGCUUCAAGGAGGAACUGGACAAGUACUUCAAGAACCACACCAGCCCCGACGUGGACCUGGGCGAC |
| | | AUCAGCGGCAUCAACGCCAGCGUGGUGAACAUCCAGAAGGAGAUCGACCGGCUGAACGAGGUGGCCAAGAACCU |
| | | GAACGAGAGCCUGAUCGACCUGCAGGAGCUGGGCAAGUACGAGCAGUACAUCAAGUGGCCCUGGUACAUCUGGC |
| | | UGGGCUUUAUUGCCGGCCUGAUCGCCAUCGUGAUGGUGACCAUCAUGCUGUGCUGUAUGACAAGCUGUUGCAGU |
| | | UGCCUGAAGGGCUGCUGCAGCUGCGGCAGCUGCUGCAAGUUCGACGAGGACGACAGCGAGCCCGUGCUGAAGG |
| | | GCGUGAAGCUGCACUACACC |
| 128 | 2A8210 | AUGGGCGUCAAGGUCCUGUUCGCCCUGAUCUGUAUUGCCGUGGCCGAGGCCAAGCGGGUGCAGCCCACCGAGAG |
| | | CAUCGUGCGGUUCCCCAACAUCACCAACCUGUGCCCCUUCGGCGAGGUGUUCAACGCCACCCGGUUCGCCAGCG |
| | | UGUACGCCUGGAACCGGAAGCGGAUCAGCAACUGCGUGGCCGACUACAGCGUGCUGUACAACAGCGCCAGCUUC |
| | | AGCACCUUCAAGUGCUACGGCGUGAGCCCCACCAAGCUGAACGACCUGUGCUUCACCAACGUGUACGCCGACAGC |
| | | UUCGUGAUCCGGGGCGACGAGGUGCGGCAGAUCGCCCCCGGCCAGACAGGCAACAUUGCUGAUUACAAUUACAA |
| | | GCUGCCCGACGAUUUCACCGGCUGCGUGAUCGCCUGGAACAGCAACAAUCUGGACAGCAAGGUGGGGGAAACU |
| | | ACAACUACCUGUACCGGCUGUUCCGCAAAAGUAACCUCAAGCCUUUCGAGCGGGACAUCAGCACCGAGAUCUACC |
| | | AGGCCGGCAGCACCCCCUGCAACGGCGUGAAGGGCUUCAACUGCUACUUCCCCCUGCAGAGCUACGGCUUCCAG |
| | | CCCACCUACGGCGUGGGCUACCAGCCCUACCGGGUGGUCGUGCUGAGCUUCGAGCUGCUCCACGCCCCCGCCAC |
| | | CGUGUGCGGCCCCAAGAAAAGCACCAACCUGGUGAAGAACAAGUGCGUGAACUUC |
| 129 | 2A9200 | AUGUUCGUGUUCCUGGUGCUGCUCCCCCUGGUGAGCUCCCAGUGCGUCAACUUUACCACCCGGACCCAGCUGCC |
| | | CCCCGCCUAUACCAACAGCUUCACCCGCGGCGUCUACUACCCCGACAAGGUGUUCCGGAGCAGCGUGCUGCACA |
| | | GCACCCAGGACCUCUUCCUCCCCUUUUUUAGCAACGUGACCUGGUUCCACGCCAUCCACGUGAGUGGCACCAACG |
| | | GCACCAAGCGGUUCGCCAACCCUGUGCUCCCCUUCAACGACGGCGUGUACUUCGCUAGCACAGAAAAAAGUAACA |
| | | UCAUUCGGGGCUGGAUCUUCGGCACCACACUGGAUAGCAAAACCCAGAGCCUGCUGAUUGUGAAUAACGCCACCA |
| | | ACGUGGUGAUCAAGGUGUGCGAGUUCCAGUUCUGCAACGACCCCUUCCUGGGCGUGUACUACCACAAGAACAACA |
| | | AGAGCUGGAUGGAGAGCGAGUUCCGCGUGUAUAGCUCUGCCAACAACUGCACCUUCGAGUACGUGAGCCAGCCC |
| | | UUCCUGAUGGACCUGGAGGGCAAGCAGGGCAAUUUCAAAAACCUGCGGGAGUUCGUGUUCAAGAACAUCGACGG |
| | | CUACUUCAAGAUCUACAGCAAGCACACCCCCAUCAACCUGGUGCGGGGCCUGCCCCAGGGCUUUAGCGCUCUGG |
| | | AGCCCCUGGUGGACCUGCCCAUCGGCAUCAACAUCACCCGCUUCCAAACCCUGCACAUCAGCUACCUGACCCCUG |
| | | GCGAUAGCAGUAGUGGCUGGACAGCCGGAGCCGCCGCCUACUACGUGGGAUACCUGCAGCCCCGGACCUUCCUG |
| | | UUGAAGUACAACGAGAACGGCACAAUCACAGACGCCGUGGACUGCGCCCUGGACCCCCUGAGCGAAACAAAGUGC |
| | | ACCCUCAAAAGUUUCACCGUGGAGAAGGGCAUCUACCAGACCAGCAACUUCCGGGUGCAGCCCACCGAGAGCAUC |
| | | GUGCGGUUCCCCAACAUCACCAACCUGUGCCCCUUCGGCGAGGUGUUCAACGCCACCCGGUUCGCCAGCGUGUA |
| | | CGCCUGGAACCGGAAGCGGAUCAGCAACUGCGUGGCCGACUACAGCGUGCUGUAUAACAGUGCCAGCUUCAGCA |
| | | CCUUCAAGUGCUACGGCGUGAGCCCCACCAAGCUGAACGACCUGUGCUUCACCAACGUGUACGCCGACAGCUUC |
| | | GUGAUCCGGGGCGACGAGGUGCGGCAGAUCGCCCCCGGCCAGACCGGCAAUAUUGCUGACUAUAACUAUAAGCU |
| | | GCCCGACGAUUUCACCGGCUGCGUGAUCGCCUGGAACAGCAACAACCUGGACAGCAAGGUGGGCGGAAACUACAA |
| | | CUACCUGUACCGGCUGUUCCGCAAAAGUAACCUCAAGCCUUUCGAGCGGGACAUCAGCACCGAGAUCUACCAGGC |
| | | CGGCAGUACCCCUUGCAACGGCGUGAAGGGCUUCAACUGCUACUUCCCCCUGCAGAGCUACGGCUUCCAGCCCA |
| | | CCUACGGCGUGGGCUACCAGCCCUACCGGGUGGUGGUUCUGAGCUUCGAGCUGCUGCACGCCCCUGCCACCGU |
| | | GUGCGGCCCCAAGAAAAGCACCAACCUGGUGAAGAACAAGUGCGUGAACUUCAACUUUAACGGACUGACAGGCAC |
| | | CGGCGUGCUGACCGAGAGCAACAAGAAGUUCCUGCCCUUCCAGCAAUUCGGCCGGGAUAUUGCCGACACCACUG |
| | | ACGCCGUGCGGGACCCCCAGACCCUGGAGAUCCUGGACAUCACACCCUGUAGCUUCGGCGGCGUGAGCGUGAUC |
| | | ACCCCCGGCACCAACACCAGCAACCAGGUGGCCGUGCUCUACCAAGGCGUGAACUGCACCGAGGUGCCCGUGGC |
| | | CAUCCACGCCGACCAGCUGACCCCCACCUGGCGGGUGUACAGCACCGGCAGCAACGUGUUCCAGACCCGGGCCG |
| | | GCUGCCUGAUCGGCGCCGAGCACGUGAAUAACAGCUACGAGUGCGACAUCCCCAUCGGAGCCGGAAUCUGCGCC |
| | | AGCUACCAGACCCAGACCAACAGCCCCCGGAGAGCCCGGAGCGUGGCCAGCCAGAGCAUUAUUGCCUACACCAUG |
| | | AGCCUGGGCGUGGAGAACAGCGUGGCCUACAGCAACAAUAGUAUUGCCAUCCCCACCAACUUCACCAUCAGCGUG |
| | | ACCACCGAAAUCCUCCCCGUGAGCAUGACCAAGACCAGCGUGGACUGCACCAUGUACAUCUGCGGCGACAGCACC |
| | | GAGUGCAGCAACCUGCUGCUCCAGUACGGCAGCUUCUGCACCCAGCUGAACCGGGCCCUGACCGGAAUUGCCGU |
| | | GGAGCAGGACAAGAACACCCAGGAGGUGUUCGCCCAGGUGAAGCAGAUCUACAAGACACCUCCCAUCAAGGACUU |
| | | CGGCGGGUUCAACUUCAGCCAGAUCCUGCCCGACCCCAGCAAGCCCAGCAAGCGGAGCUUCAUCGAGGACCUGC |
| | | UGUUCAACAAGGUGACCCUGGCCGACGCCGGCUUCAUCAAGCAGUACGGCGACUGUCUGGGAGAUAUUGCCGCA |
| | | CGGGACCUGAUCUGCGCCCAGAAGUUCAACGGCCUGACCGUGCUGCCCCCACUGUUGACCGACGAGAUGAUCGC |
| | | CCAGUACACAAGCGCUCUGCUGGCCGGCACCAUCACCAGCGGCUGGACCUUCGGCGCCGGCGCUGCCCUGCAGA |
| | | UCCCCUUCGCCAUGCAGAUGGCCUACCGGUUCAACGGCAUCGGCGUGACCCAGAACGUGCUGUACGAGAACCAG |
| | | AAGCUGAUCGCCAACCAGUUCAACAGCGCCAUCGGCAAGAUCCAGGACAGCCUGAGCUCCACCGCCAGCGCCCUG |
| | | GGCAAGCUGCAGGACGUGGUGAACCAGAACGCCCAGGCCCUGAACACCCUGGUGAAGCAGCUGAGCAGCAACUU |
| | | CGGCGCCAUCAGCUCUGUGCUGAACGACAUCCUGAGCCGGCUGGAUCCUCCUGAGGCCGAGGUGCAAAUCGAUC |
| | | GGCUCAUCACCGGCCGGCUGCAGAGCCUGCAGACCUACGUGACACAGCAGCUGAUCCGGGCCGCCGAGAUCCGG |
| | | GCCAGCGCCAACCUGGCCGCUACCAAGAUGAGCGAGUGCGUGCUGGGCCAGAGCAAGCGGGUGGACUUCUGCGG |
| | | CAAGGGCUACCACCUGAUGAGCUUCCCCCAGAGCGCCCCCCACGGCGUGGUGUUCCUGCACGUGACCUACGUGC |
| | | CCGCCCAGGAGAAGAACUUCACCACCGCCCCCGCCAUCUGCCACGACGGCAAGGCCCACUUCCCCCGGGAGGGC |
| | | GUGUUCGUGAGCAACGGCACCCACUGGUUCGUGACCCAGCGGAACUUCUACGAGCCCCAGAUCAUAACCACAGAC |
| | | AACACCUUCGUGAGCGGCAACUGCGACGUGGUCAUCGGCAUCGUGAACAACACCGUGUACGACCCCCUGCAGCC |
| | | CGAGCUGGACAGCUUCAAGGAGGAACUGGACAAGUACUUCAAGAACCACACCAGCCCCGACGUGGACCUGGGCGA |
| | | CAUCAGCGGCAUCAACGCCAGCGUGGUGAACAUCCAGAAGGAGAUCGACCGGCUGAACGAGGUGGCCAAGAACCU |
| | | GAACGAGAGCCUGAUCGACCUGCAGGAGCUGGGCAAGUACGAGCAGUACAUCAAGUGGCCCUGGUACAUCUGGC |
| | | UGGGCUUUAUUGCCGGCCUGAUCGCCAUCGUGAUGGUGACCAUCAUGCUGUGCUGUAUGACAAGCUGUUGCAGU |
| | | UGCCUGAAGGGCUGCUGCAGCUGCGGCAGCUGCUGCAAGUUCGACGAGGACGACAGCGAGCCCGUGCUGAAGG |
| | | GCGUGAAGCUGCACUACACC |
| 130 | 2A7020 | AUGUUCGUGUUCCUGGUGCUGCUGCCUCUGGUGUCCAGCCAGUGUGUGAACCUGACCACCAGAACACAGCUGCC |
| | | UCCAGCCUACACCAACAGCUUUACCAGAGGCGUGUACUACCCCGACAAGGUGUUCAGAUCCAGCGUGCUGCACUC |
| | | UACCCAGGACCUGUUCCUGCCUUUCUUCAGCAACGUGACCUGGUUCCACGCCAUCCACGUGUCCGGCACCAAUG |
| | | GCACCAAGAGAUUCGACAACCCCGUGCUGCCCUUCAACGACGGGGUGUACUUUGCCAGCACCGAGAAGUCCAACA |
| | | UCAUCAGAGGCUGGAUCUUCGGCACCACACUGGACAGCAAGACCCAGAGCCUGCUGAUCGUGAACAACGCCACCA |
| | | ACGUGGUCAUCAAAGUGUGCGAGUUCCAGUUCUGCAACGACCCCUUCCUGGGCGUCUACUACCACAAGAACAACA |
| | | AGAGCUGGAUGGAAAGCGAGUUCCGGGUGUACAGCAGCGCCAACAACUGCACCUUCGAGUACGUGUCCCAGCCU |
| | | UUCCUGAUGGACCUGGAAGGCAAGCAGGGCAACUUCAAGAACCUGCGCGAGUUCGUGUUUAAGAACAUCGACGG |
| | | CUACUUCAAGAUCUACAGCAAGCACACCCCUAUCAACCUCGUGCGGGAUCUGCCUCAGGGCUUCUCUGCUCUGGA |
| | | ACCCCUGGUGGAUCUGCCCAUCGGCAUCAACAUCACCCGGUUUCAGACACUGCUGGCCCUGCACAGAAGCUACCU |
| | | GACACCUGGCGAUAGCAGCAGCGGAUGGACAGCUGGUGCCGCCGCUUACUAUGUGGGCUACCUGCAGCCUAGAA |
| | | CCUUCCUGCUGAAGUACAACGAGAACGGCACCAUCACCGACGCCGUGGAUUGUGCUCUGGAUCCUCUGAGCGAG |
| | | ACAAAGUGCACCCUGAAGUCCUUCACCGUGGAAAAGGGCAUCUACCAGACCAGCAACUUCCGGGUGCAGCCCACC |
| | | GAAUCCAUCGUGCGGUUCCCCAAUAUCACCAAUCUGUGCCCCUUCGGCGAGGUGUUCAAUGCCACCAGAUUCGCC |
| | | UCUGUGUACGCCUGGAACCGGAAGCGGAUCAGCAAUUGCGUGGCCGACUACUCCGUGCUGUACAACUCCGCCAG |
| | | CUUCAGCACCUUCAAGUGCUACGGCGUGUCCCCUACCAAGCUGAACGACCUGUGCUUCACAAACGUGUACGCCGA |
| | | CAGCUUCGUGAUCCGGGGAGAUGAAGUGCGGCAGAUUGCCCCUGGACAGACAGGCAAGAUCGCCGACUACAACU |
| | | ACAAGCUGCCCGACGACUUCACCGGCUGUGUGAUUGCCUGGAACAGCAACAACCUGGACUCCAAAGUCGGCGGCA |
| | | ACUACAAUUACCUGUACCGGCUGUUCCGGAAGUCCAAUCUGAAGCCCUUCGAGCGGGACAUCUCCACCGAGAUCU |
| | | AUCAGGCCGGCAGCACCCCUUGUAACGGCGUGGAAGGCUUCAACUGCUACUUCCCACUGCAGUCCUACGGCUUU |
| | | CAGCCCACAAAUGGCGUGGGCUAUCAGCCCUACAGAGUGGUGGUGCUGAGCUUCGAACUGCUGCAUGCCCCUGC |
| | | CACAGUGUGCGGCCCUAAGAAAAGCACCAAUCUCGUGAAGAACAAAUGCGUGAACUUCAACUUCAACGGCCUGAC |
| | | CGGCACCGGCGUGCUGACAGAGAGCAACAAGAAGUUCCUGCCAUUCCAGCAGUUUGGCCGGGAUAUCGCCGAUA |
| | | CCACAGACGCCGUUAGAGAUCCCCAGACACUGGAAAUCCUGGACAUCACCCCUUGCAGCUUCGGCGGAGUGUCU |
| | | GUGAUCACCCCUGGCACCAACACCAGCAAUCAGGUGGCAGUGCUGUACCAGGACGUGAACUGUACCGAAGUGCCC |
| | | GUGGCCAUUCACGCCGAUCAGCUGACACCUACAUGGCGGGUGUACUCCACCGGCAGCAAUGUGUUUCAGACCAG |
| | | AGCCGGCUGUCUGAUCGGAGCCGAGCACGUGAACAAUAGCUACGAGUGCGACAUCCCCAUCGGCGCUGGAAUCU |
| | | GCGCCAGCUACCAGACACAGACAAACAGCCCUCGGAGAGCCAGAAGCGUGGCCAGCCAGAGCAUCAUUGCCUACA |
| | | CAAUGUCUCUGGGCGCCGAGAACAGCGUGGCCUACUCCAACAACUCUAUCGCUAUCCCCACCAACUUCACCAUCA |
| | | GCGUGACCACAGAGAUCCUGCCUGUGUCCAUGACCAAGACCAGCGUGGACUGCACCAUGUACAUCUGCGGCGAU |
| | | UCCACCGAGUGCUCCAACCUGCUGCUGCAGUACGGCAGCUUCUGCACCCAGCUGAAUAGAGCCCUGACAGGGAU |
| | | CGCCGUGGAACAGGACAAGAACACCCAAGAGGUGUUCGCCCAAGUGAAGCAGAUCUACAAGACCCCUCCUAUCAA |
| | | GGACUUCGGCGGCUUCAAUUUCAGCCAGAUUCUGCCCGAUCCUAGCAAGCCCAGCAAGCGGAGCUUCAUCGAGG |
| | | ACCUGCUGUUCAACAAAGUGACACUGGCCGACGCCGGCUUCAUCAAGCAGUAUGGCGAUUGUCUGGGCGACAUU |
| | | GCCGCCAGGGAUCUGAUUUGCGCCCAGAAGUUUAACGGACUGACAGUGCUGCCUCCUCUGCUGACCGAUGAGAU |
| | | GAUCGCCCAGUACACAUCUGCCCUGCUGGCCGGCACAAUCACAAGCGGCUGGACAUUUGGAGCAGGCGCCGCUC |
| | | UGCAGAUCCCCUUUGCUAUGCAGAUGGCCUACCGGUUCAACGGCAUCGGAGUGACCCAGAAUGUGCUGUACGAG |
| | | AACCAGAAGCUGAUCGCCAACCAGUUCAACAGCGCCAUCGGCAAGAUCCAGGACAGCCUGAGCAGCACAGCAAGC |
| | | GCCCUGGGAAAGCUGCAGGACGUGGUCAACCAGAAUGCCCAGGCACUGAACACCCUGGUCAAGCAGCUGUCCUC |
| | | CAACUUCGGCGCCAUCAGCUCUGUGCUGAACGAUAUCCUGAGCAGACUGGACCCUCCUGAGGCCGAGGUGCAGA |
| | | UCGACAGACUGAUCACAGGCAGACUGCAGAGCCUCCAGACAUACGUGACCCAGCAGCUGAUCAGAGCCGCCGAGA |
| | | UUAGAGCCUCUGCCAAUCUGGCCGCCACCAAGAUGUCUGAGUGUGUGCUGGGCCAGAGCAAGAGAGUGGACUUU |
| | | UGCGGCAAGGGCUACCACCUGAUGAGCUUCCCUCAGUCUGCCCCUCACGGCGUGGUGUUUCUGCACGUGACAUA |
| | | UGUGCCCGCUCAAGAGAAGAAUUUCACCACCGCUCCAGCCAUCUGCCACGACGGCAAAGCCCACUUUCCUAGAGA |
| | | AGGCGUGUUCGUGUCCAACGGCACCCAUUGGUUCGUGACACAGCGGAACUUCUACGAGCCCCAGAUCAUCACCAC |
| | | CGACAACACCUUCGUGUCUGGCAACUGCGACGUCGUGAUCGGCAUUGUGAACAAUACCGUGUACGACCCUCUGCA |
| | | GCCCGAGCUGGACAGCUUCAAAGAGGAACUGGACAAGUACUUUAAGAACCACACAAGCCCCGACGUGGACCUGGG |
| | | CGAUAUCAGCGGAAUCAAUGCCAGCGUCGUGAACAUCCAGAAAGAGAUCGACCGGCUGAACGAGGUGGCCAAGAA |
| | | UCUGAACGAGAGCCUGAUCGACCUGCAAGAACUGGGGAAGUACGAGCAGUACAUCAAGUGGCCCUGGUACAUCUG |
| | | GCUGGGCUUUAUCGCCGGACUGAUUGCCAUCGUGAUGGUCACAAUCAUGCUGUGUUGCAUGACCAGCUGCUGUA |
| | | GCUGCCUGAAGGGCUGUUGUAGCUGUGGCAGCUGCUGCAAGUUCGACGAGGACGAUUCUGAGCCCGUGCUGAAG |
| | | GGCGUGAAACUGCACUACACA |
| 131 | 2A8620 | AUGUUUGUUUUUCUUGUUUUAUUGCCACUAGUCUCUAGUCAGUGUGUUAAUCUUACAACCAGAACUCAAUUACCC |
| | | CCUGCAUACACUAAUUCUUUCACACGUGGUGUUUAUUACCCUGACAAAGUUUUCAGAUCCUCAGUUUUACAUUCA |
| | | ACUCAGGAUUUGUUCUUACCUUUCUUUUCCAAUGUUACUUGGUUCCAUGCUAUACAUGUCUCUGGGACCAAUGGU |
| | | ACUAAGAGGUUUGAUAACCCUGUCCUACCAUUUAAUGAUGGUGUUUAUUUUGCUUCCACUGAGAAGUCUAACAUA |
| | | AUAAGAGGCUGGAUCUUUGGUACUACUUUAGAUUCGAAGACCCAGUCCCUACUUAUUGUUAAUAACGCUACUAAU |
| | | GUUGUUAUUAAAGUCUGUGAAUUUCAAUUUUGUAAUGAUCCAUUUUUGGGUGUUUAUUACCACAAAAACAACAAAA |
| | | GUUGGAUGGAAAGUGAGUUCAGAGUUUAUUCUAGUGCGAAUAAUUGCACUUUUGAAUAUGUCUCUCAGCCUUUUC |
| | | UUAUGGACCUUGAAGGAAAACAGGGUAAUUUCAAAAAUCUUAGGGAAUUUGUGUUUAAGAAUAUUGAUGGUUAUU |
| | | UUAAAAUAUAUUCUAAGCACACGCCUAUUAAUUUAGUGCGUGAUCUCCCUCAGGGUUUUUCGGCUUUAGAACCAU |
| | | UGGUAGAUUUGCCAAUAGGUAUUAACAUCACUAGGUUUCAAACUUUACUUGCUUUACAUAGAAGUUAUUUGACUC |
| | | CUGGUGAUUCUUCUUCAGGUUGGACAGCUGGUGCUGCAGCUUAUUAUGUGGGUUAUCUUCAACCUAGGACUUUU |
| | | CUAUUAAAAUAUAAUGAAAAUGGAACCAUUACAGAUGCUGUAGACUGUGCACUUGACCCUCUCUCAGAAACAAAGU |
| | | GUACGUUGAAAUCCUUCACUGUAGAAAAAGGAAUCUAUCAAACUUCUAACUUUAGAGUCCAACCAACAGAAUCUAU |
| | | UGUUAGAUUUCCUAAUAUUACAAACUUGUGCCCUUUUGGUGAAGUUUUUAACGCCACCAGAUUUGCAUCCGUGUA |
| | | UGCUUGGAACAGGAAGAGAAUCAGCAACUGUGUUGCUGAUUAUUCUGUCCUAUAUAAUUCCGCAUCAUUUUCCAC |
| | | UUUUAAGUGUUAUGGAGUGUCUCCUACUAAAUUAAAUGAUCUCUGCUUUACUAAUGUCUAUGCAGAUUCAUUUGU |
| | | AAUUAGAGGUGAUGAAGUCAGACAAAUCGCUCCAGGGCAAACUGGAAAGAUUGCUGAUUAUAAUUAUAAAUUACCA |
| | | GAUGAUUUUACAGGCUGCGUUAUAGCUUGGAAUUCUAACAAUCUUGAUUCUAAGGUUGGUGGUAAUUAUAAUUAC |
| | | CUGUAUAGAUUGUUUAGGAAGUCUAAUCUCAAACCUUUUGAGAGAGAUAUUUCAACUGAAAUCUAUCAGGCCGGU |
| | | AGCACACCUUGUAAUGGUGUUGAAGGUUUUAAUUGUUACUUUCCUUUACAAUCAUAUGGUUUCCAACCCACUAAU |
| | | GGUGUUGGUUACCAACCAUACAGAGUAGUAGUACUUUCUUUUGAACUUCUACAUGCACCAGCAACUGUUUGUGGA |
| | | CCUAAAAAGUCUACUAAUUUGGUUAAAAACAAAUGUGUCAAUUUCAACUUCAAUGGUUUAACAGGCACAGGUGUUC |
| | | UUACUGAGUCUAACAAAAAGUUUCUGCCUUUCCAACAAUUUGGCAGAGACAUUGCUGACACUACUGAUGCUGUCC |
| | | GUGAUCCACAGACACUUGAGAUUCUUGACAUUACACCAUGUUCUUUUGGUGGUGUCAGUGUUAUAACACCAGGAA |
| | | CAAAUACUUCUAACCAGGUUGCUGUUCUUUAUCAGGAUGUUAACUGCACAGAAGUCCCUGUUGCUAUUCAUGCAG |
| | | AUCAACUUACUCCUACUUGGCGUGUUUAUUCUACAGGUUCUAAUGUUUUUCAAACACGUGCAGGCUGUUUAAUAG |
| | | GGGCUGAACAUGUCAACAACUCAUAUGAGUGUGACAUACCCAUUGGUGCAGGUAUAUGCGCUAGUUAUCAGACUC |
| | | AGACUAAUUCUCCUCGGCGGGCACGUAGUGUAGCUAGUCAAUCCAUCAUUGCCUACACUAUGUCACUUGGUGCAG |
| | | AAAAUUCAGUUGCUUACUCUAAUAACUCUAUUGCCAUACCCACAAAUUUUACUAUUAGUGUUACCACAGAAAUUCU |
| | | ACCAGUGUCUAUGACCAAGACAUCAGUAGAUUGUACAAUGUACAUUUGUGGUGAUUCAACUGAAUGCAGCAAUCU |
| | | UUUGUUGCAAUAUGGCAGUUUUUGUACACAAUUAAACCGUGCUUUAACUGGGAUAGCUGUUGAACAAGACAAAAA |
| | | CACCCAAGAAGUUUUUGCACAAGUCAAACAAAUUUACAAAACACCACCAAUUAAAGAUUUUGGUGGUUUUAAUUUU |
| | | UCACAAAUAUUACCAGAUCCAUCAAAACCAAGCAAGAGGUCAUUUAUUGAAGAUCUACUUUUCAACAAAGUGACAC |
| | | UUGCAGAUGCUGGCUUCAUCAAACAAUAUGGUGAUUGCCUUGGUGAUAUUGCUGCUAGGGACCUCAUUUGUGCA |
| | | CAAAAGUUUAACGGCCUUACUGUUUUGCCACCUUUGCUCACAGAUGAAAUGAUUGCUCAAUACACUUCUGCACUG |
| | | UUAGCGGGUACAAUCACUUCUGGUUGGACCUUUGGUGCAGGUGCUGCAUUACAAAUACCAUUUGCUAUGCAAAUG |
| | | GCUUAUAGGUUUAAUGGUAUUGGAGUUACACAGAAUGUUCUCUAUGAGAACCAAAAAUUGAUUGCCAACCAAUUUA |
| | | AUAGUGCCAUUGGCAAAAUUCAAGACUCACUUUCUUCCACAGCAAGUGCACUUGGAAAACUUCAAGAUGUGGUCA |
| | | ACCAAAAUGCACAAGCUUUAAACACGCUUGUUAAACAACUUAGCUCCAAUUUUGGUGCAAUUUCAAGUGUUUUAAA |
| | | UGAUAUCCUUUCACGUCUUGACCCUCCCGAGGCUGAAGUGCAAAUUGAUAGGUUGAUCACAGGCAGACUUCAAAG |
| | | UUUGCAGACAUAUGUGACUCAACAAUUAAUUAGAGCUGCAGAAAUCAGAGCUUCUGCUAAUCUUGCUGCUACUAAA |
| | | AUGUCAGAGUGUGUACUUGGACAAUCAAAAAGAGUUGAUUUUUGUGGAAAGGGCUAUCAUCUUAUGUCCUUCCCU |
| | | CAGUCAGCACCUCAUGGUGUAGUCUUCUUGCAUGUGACUUAUGUCCCUGCACAAGAAAAGAACUUCACAACUGCU |
| | | CCUGCCAUUUGUCAUGAUGGAAAAGCACACUUUCCUCGUGAAGGUGUCUUUGUUUCAAAUGGCACACACUGGUUU |
| | | GUAACACAAAGGAAUUUUUAUGAACCACAAAUCAUUACUACAGACAACACAUUUGUGUCUGGUAACUGUGAUGUUG |
| | | UAAUAGGAAUUGUCAACAACACAGUUUAUGAUCCUUUGCAACCUGAAUUAGACUCAUUCAAGGAGGAGUUAGAUAA |
| | | AUAUUUUAAGAAUCAUACAUCACCAGAUGUUGAUUUAGGUGACAUCUCUGGCAUUAAUGCUUCAGUUGUAAACAUU |
| | | CAAAAAGAAAUUGACCGCCUCAAUGAGGUUGCCAAGAAUUUAAAUGAAUCUCUCAUCGAUCUCCAAGAACUUGGAA |
| | | AGUAUGAGCAGUAUAUAAAAUGGCCAUGGUACAUUUGGCUAGGUUUUAUAGCUGGCUUGAUUGCCAUAGUAAUGG |
| | | UGACAAUUAUGCUUUGCUGUAUGACCAGUUGCUGUAGUUGUCUCAAGGGCUGUUGUUCUUGUGGAUCCUGCUGC |
| | | AAAUUUGAUGAAGACGACUCUGAGCCAGUGCUCAAAGGAGUCAAAUUACAUUACACA |
| 132 | 2A9420 | AUGUUCGUGUUCCUGGUGCUGCUGCCUCUGGUGUCCAGCCAGUGUGUGAACCUGACCACCAGAACACAGCUGCC |
| | | UCCAGCCUACACCAACAGCUUUACCAGAGGCGUGUACUACCCCGACAAGGUGUUCAGAUCCAGCGUGCUGCACUC |
| | | UACCCAGGACCUGUUCCUGCCUUUCUUCAGCAACGUGACCUGGUUCCACGCCAUCCACGUGUCCGGCACCAACG |
| | | GCACCAAGAGAUUCGACAACCCCGUGCUGCCCUUCAACGACGGGGUGUACUUUGCCAGCACCGAGAAGUCCAACA |
| | | UCAUCAGAGGCUGGAUCUUCGGCACCACACUGGACAGCAAGACCCAGAGCCUGCUGAUCGUGAACAACGCCACCA |
| | | ACGUGGUCAUCAAAGUGUGCGAGUUCCAGUUCUGCAACGACCCCUUCCUGGGCGUCUACUACCACAAGAACAACA |
| | | AGAGCUGGAUGGAAAGCGAGUUCCGGGUGUACAGCAGCGCCAACAACUGCACCUUCGAGUACGUGUCCCAGCCU |
| | | UUCCUGAUGGACCUGGAAGGCAAGCAGGGCAACUUCAAGAACCUGCGCGAGUUCGUGUUUAAGAACAUCGACGG |
| | | CUACUUCAAGAUCUACAGCAAGCACACCCCUAUCAACCUCGUGCGGGAUCUGCCUCAGGGCUUCUCUGCUCUGGA |
| | | ACCCCUGGUGGAUCUGCCCAUCGGCAUCAACAUCACCCGGUUUCAGACACUGCUGGCCCUGCACAGAAGCUACCU |
| | | GACACCUGGCGAUAGCAGCAGCGGCUGGACAGCUGGUGCCGCCGCUUACUACGUGGGCUACCUGCAGCCUAGAA |
| | | CCUUCCUGCUGAAGUACAACGAGAACGGCACCAUCACCGACGCCGUGGAUUGUGCUCUGGAUCCUCUGAGCGAG |
| | | ACAAAGUGCACCCUGAAGUCCUUCACCGUGGAAAAGGGCAUCUACCAGACCAGCAACUUCCGGGUGCAGCCCACC |
| | | GAAUCCAUCGUGCGGUUCCCCAAUAUCACCAAUCUGUGCCCCUUCGGCGAGGUGUUCAACGCCACCAGAUUCGCC |
| | | UCUGUGUACGCCUGGAACCGGAAGCGGAUCAGCAAUUGCGUGGCCGACUACUCCGUGCUGUACAACUCCGCCAG |
| | | CUUCAGCACCUUCAAGUGCUACGGCGUGUCCCCUACCAAGCUGAACGACCUGUGCUUCACAAACGUGUACGCCGA |
| | | CAGCUUCGUGAUCCGGGGAGACGAAGUGCGGCAGAUUGCCCCUGGACAGACAGGCAAGAUCGCCGACUACAACU |
| | | ACAAGCUGCCCGACGACUUCACCGGCUGUGUGAUUGCCUGGAACAGCAACAACCUGGACUCCAAAGUCGGCGGCA |
| | | ACUACAAUUACCUGUACCGGCUGUUCCGGAAGUCCAAUCUGAAGCCCUUCGAGCGGGACAUCUCCACCGAGAUCU |
| | | AUCAGGCCGGCAGCACCCCUUGUAACGGCGUGGAAGGCUUCAACUGCUACUUCCCACUGCAGUCCUACGGCUUU |
| | | CAGCCCACAAACGGCGUGGGCUAUCAGCCCUACAGAGUGGUGGUGCUGAGCUUCGAACUGCUGCACGCCCCUGC |
| | | CACAGUGUGCGGCCCUAAGAAAAGCACCAAUCUCGUGAAGAACAAGUGCGUGAACUUCAACUUCAACGGCCUGAC |
| | | CGGCACCGGCGUGCUGACAGAGAGCAACAAGAAGUUCCUGCCAUUCCAGCAGUUUGGCCGGGAUAUCGCCGAUA |
| | | CCACAGACGCCGUUAGAGAUCCCCAGACACUGGAAAUCCUGGACAUCACCCCUUGCAGCUUCGGCGGAGUGUCU |
| | | GUGAUCACCCCUGGCACCAACACCAGCAAUCAGGUGGCAGUGCUGUACCAGGACGUGAACUGUACCGAAGUGCCC |
| | | GUGGCCAUUCACGCCGAUCAGCUGACACCUACCUGGCGGGUGUACUCCACCGGCAGCAACGUGUUUCAGACCAG |
| | | AGCCGGCUGUCUGAUCGGAGCCGAGCACGUGAACAAUAGCUACGAGUGCGACAUCCCCAUCGGCGCUGGAAUCU |
| | | GCGCCAGCUACCAGACACAGACAAACAGCCCUCGGAGAGCCAGAAGCGUGGCCAGCCAGAGCAUCAUUGCCUACA |
| | | CAAUGUCUCUGGGCGCCGAGAACAGCGUGGCCUACUCCAACAACUCUAUCGCUAUCCCCACCAACUUCACCAUCA |
| | | GCGUGACCACAGAGAUCCUGCCUGUGUCCAUGACCAAGACCAGCGUGGACUGCACCAUGUACAUCUGCGGCGAU |
| | | UCCACCGAGUGCUCCAACCUGCUGCUGCAGUACGGCAGCUUCUGCACCCAGCUGAAUAGAGCCCUGACAGGGAU |
| | | CGCCGUGGAACAGGACAAGAACACCCAAGAGGUGUUCGCCCAAGUGAAGCAGAUCUACAAGACCCCUCCUAUCAA |
| | | GGACUUCGGCGGCUUCAAUUUCAGCCAGAUUCUGCCCGAUCCUAGCAAGCCCAGCAAGCGGAGCUUCAUCGAGG |
| | | ACCUGCUGUUCAACAAAGUGACACUGGCCGACGCCGGCUUCAUCAAGCAGUACGGCGAUUGUCUGGGCGACAUU |
| | | GCCGCCAGGGAUCUGAUUUGCGCCCAGAAGUUUAACGGACUGACAGUGCUGCCUCCUCUGCUGACCGACGAGAU |
| | | GAUCGCCCAGUACACAUCUGCCCUGCUGGCCGGCACAAUCACAAGCGGCUGGACAUUUGGAGCAGGCGCCGCUC |
| | | UGCAGAUCCCCUUUGCUAUGCAGAUGGCCUACCGGUUCAACGGCAUCGGAGUGACCCAGAACGUGCUGUACGAG |
| | | AACCAGAAGCUGAUCGCCAACCAGUUCAACAGCGCCAUCGGCAAGAUCCAGGACAGCCUGAGCAGCACAGCAAGC |
| | | GCCCUGGGAAAGCUGCAGGACGUGGUCAACCAGAACGCCCAGGCACUGAACACCCUGGUCAAGCAGCUGUCCUC |
| | | CAACUUCGGCGCCAUCAGCUCUGUGCUGAACGAUAUCCUGAGCAGACUGGACCCUCCUGAGGCCGAGGUGCAGA |
| | | UCGACAGACUGAUCACAGGCAGACUGCAGAGCCUCCAGACAUACGUGACCCAGCAGCUGAUCAGAGCCGCCGAGA |
| | | UUAGAGCCUCUGCCAAUCUGGCCGCCACCAAGAUGUCUGAGUGUGUGCUGGGCCAGAGCAAGAGAGUGGACUUU |
| | | UGCGGCAAGGGCUACCACCUGAUGAGCUUCCCUCAGUCUGCCCCUCACGGCGUGGUGUUUCUGCACGUGACAUA |
| | | CGUGCCCGCUCAAGAGAAGAAUUUCACCACCGCUCCAGCCAUCUGCCACGACGGCAAAGCCCACUUUCCUAGAGA |
| | | AGGCGUGUUCGUGUCCAACGGCACCCAUUGGUUCGUGACACAGCGGAACUUCUACGAGCCCCAGAUCAUCACCAC |
| | | CGACAACACCUUCGUGUCUGGCAACUGCGACGUCGUGAUCGGCAUUGUGAACAAUACCGUGUACGACCCUCUGCA |
| | | GCCCGAGCUGGACAGCUUCAAAGAGGAACUGGACAAGUACUUUAAGAACCACACAAGCCCCGACGUGGACCUGGG |
| | | CGAUAUCAGCGGAAUCAACGCCAGCGUCGUGAACAUCCAGAAAGAGAUCGACCGGCUGAACGAGGUGGCCAAGAA |
| | | UCUGAACGAGAGCCUGAUCGACCUGCAAGAACUGGGGAAGUACGAGCAGUACAUCAAGUGGCCCUGGUACAUCUG |
| | | GCUGGGCUUUAUCGCCGGACUGAUUGCCAUCGUGAUGGUCACAAUCAUGCUGUGUUGCAUGACCAGCUGCUGUA |
| | | GCUGCCUGAAGGGCUGUUGUAGCUGUGGCAGCUGCUGCAAGUUCGACGAGGACGAUUCUGAGCCCGUGCUGAAG |
| | | GGCGUGAAACUGCACUACACA |
| 133 | 2A4361 | AUGGGAGUCAAAGUUCUGUUUGCCCUGAUCUGCAUUGCUGUGGCCGAGGCCAAGAGAGUCCAACCAACAGAAUCU |
| | | AUUGUUAGAUUUCCUAAUAUUACAAACUUGUGCCCUUUUGGUGAAGUUUUUAACGCCACCAGAUUUGCAUCCGUG |
| | | UAUGCUUGGAACAGGAAGAGAAUCAGCAACUGUGUUGCUGAUUAUUCUGUCCUAUAUAAUUCCGCAUCAUUUUCC |
| | | ACUUUUAAGUGUUAUGGAGUGUCUCCUACUAAAUUAAAUGAUCUCUGCUUUACUAAUGUCUAUGCAGAUUCAUUU |
| | | GUAAUUAGAGGUGAUGAAGUCAGACAAAUCGCUCCAGGGCAAACUGGAAAGAUUGCUGAUUAUAAUUAUAAAUUAC |
| | | CAGAUGAUUUUACAGGCUGCGUUAUAGCUUGGAAUUCUAACAAUCUUGAUUCUAAGGUUGGUGGUAAUUAUAAUU |
| | | ACCUGUAUAGAUUGUUUAGGAAGUCUAAUCUCAAACCUUUUGAGAGAGAUAUUUCAACUGAAAUCUAUCAGGCCG |
| | | GUAGCACACCUUGUAAUGGUGUUGAAGGUUUUAAUUGUUACUUUCCUUUACAAUCAUAUGGUUUCCAACCCACUA |
| | | AUGGUGUUGGUUACCAACCAUACAGAGUAGUAGUACUUUCUUUUGAACUUCUACAUGCACCAGCAACUGUUUGUG |
| | | GACCUAAAAAGUCUACUAAUUUGGUUAAAAACAAAUGUGUCAAUUUCGGAAGCGGCAGGCUGAGACGAGGCAGCG |
| | | GCGCCACCAACUUCAGCCUGUUGAAGCAGGCCGGCGACGUGGAGGAAAACCCCGGCCCCAUGGGAGUCAAAGUU |
| | | CUGUUUGCCCUGAUCUGCAUUGCUGUGGCCGAGGCCAAGAGAGUCCAACCAACAGAAUCUAUUGUUAGAUUUCCU |
| | | AAUAUUACAAACUUGUGCCCUUUUGGUGAAGUUUUUAACGCCACCAGAUUUGCAUCCGUGUAUGCUUGGAACAGG |
| | | AAGAGAAUCAGCAACUGUGUUGCUGAUUAUUCUGUCCUAUAUAAUUCCGCAUCAUUUUCCACUUUUAAGUGUUAU |
| | | GGAGUGUCUCCUACUAAAUUAAAUGAUCUCUGCUUUACUAAUGUCUAUGCAGAUUCAUUUGUAAUUAGAGGUGAU |
| | | GAAGUCAGACAAAUCGCUCCAGGGCAAACUGGAAAGAUUGCUGAUUAUAAUUAUAAAUUACCAGAUGAUUUUACAG |
| | | GCUGCGUUAUAGCUUGGAAUUCUAACAAUCUUGAUUCUAAGGUUGGUGGUAAUUAUAAUUACCUGUAUAGAUUGU |
| | | UUAGGAAGUCUAAUCUCAAACCUUUUGAGAGAGAUAUUUCAACUGAAAUCUAUCAGGCCGGUAGCACACCUUGUA |
| | | AUGGUGUUGAAGGUUUUAAUUGUUACUUUCCUUUACAAUCAUAUGGUUUCCAACCCACUAAUGGUGUUGGUUACC |
| | | AACCAUACAGAGUAGUAGUACUUUCUUUUGAACUUCUACAUGCACCAGCAACUGUUUGUGGACCUAAAAAGUCUAC |
| | | UAAUUUGGUUAAAAACAAAUGUGUCAAUUUC |
| 134 | 2A5361 | AUGGGAGUCAAAGUUCUGUUUGCCCUGAUCUGCAUUGCUGUGGCCGAGGCCAAGAGAGUCCAACCAACAGAAUCU |
| | | AUUGUUAGAUUUCCUAAUAUUACAAACUUGUGCCCUUUUGGUGAAGUUUUUAACGCCACCAGAUUUGCAUCCGUG |
| | | UAUGCUUGGAACAGGAAGAGAAUCAGCAACUGUGUUGCUGAUUAUUCUGUCCUAUAUAAUUCCGCAUCAUUUUCC |
| | | ACUUUUAAGUGUUAUGGAGUGUCUCCUACUAAAUUAAAUGAUCUCUGCUUUACUAAUGUCUAUGCAGAUUCAUUU |
| | | GUAAUUAGAGGUGAUGAAGUCAGACAAAUCGCUCCAGGGCAAACUGGAAAGAUUGCUGAUUAUAAUUAUAAAUUAC |
| | | CAGAUGAUUUUACAGGCUGCGUUAUAGCUUGGAAUUCUAACAAUCUUGAUUCUAAGGUUGGUGGUAAUUAUAAUU |
| | | ACCUGUAUAGAUUGUUUAGGAAGUCUAAUCUCAAACCUUUUGAGAGAGAUAUUUCAACUGAAAUCUAUCAGGCCG |
| | | GUAGCACACCUUGUAAUGGUGUUGAAGGUUUUAAUUGUUACUUUCCUUUACAAUCAUAUGGUUUCCAACCCACUA |
| | | AUGGUGUUGGUUACCAACCAUACAGAGUAGUAGUACUUUUUUUGAACUUCUACAUGCACCAGCAACUGUUUGUG |
| | | GACCUAAAAAGUCUACUAAUUUGGUUAAAAACAAAUGUGUCAAUUUCGGAAGCGGCAGGCUGAGACGAGGCAGCG |
| | | GCGCCACCAACUUCAGCCUGUUGAAGCAGGCCGGCGACGUGGAGGAAAACCCCGGCCCCAUGGGAGUCAAAGUU |
| | | CUGUUUGCCCUGAUCUGCAUUGCUGUGGCCGAGGCCAAGAGAGUCCAACCAACAGAAUCUAUUGUUAGAUUUCCU |
| | | AAUAUUACAAACUUGUGCCCUUUUGGUGAAGUUUUUAACGCCACCAGAUUUGCAUCCGUGUAUGCUUGGAACAGG |
| | | AAGAGAAUCAGCAACUGUGUUGCUGAUUAUUCUGUCCUAUAUAAUUCCGCAUCAUUUUCCACUUUUAAGUGUUAU |
| | | GGAGUGUCUCCUACUAAAUUAAAUGAUCUCUGCUUUACUAAUGUCUAUGCAGAUUCAUUUGUAAUUAGAGGUGAU |
| | | GAAGUCAGACAAAUCGCUCCAGGGCAAACUGGAAAGAUUGCUGAUUAUAAUUAUAAAUUACCAGAUGAUUUUACAG |
| | | GCUGCGUUAUAGCUUGGAAUUCUAACAAUCUUGAUUCUAAGGUUGGUGGUAAUUAUAAUUACCUGUAUAGAUUGU |
| | | UUAGGAAGUCUAAUCUCAAACCUUUUGAGAGAGAUAUUUCAACUGAAAUCUAUCAGGCCGGUAGCACACCUUGUA |
| | | AUGGUGUUGAAGGUUUUAAUUGUUACUUUCCUUUACAAUCAUAUGGUUUCCAACCCACUAAUGGUGUUGGUUACC |
| | | AACCAUACAGAGUAGUAGUACUUUCUUUUGAACUUCUACAUGCACCAGCAACUGUUUGUGGACCUAAAAAGUCUAC |
| | | UAAUUUGGUUAAAAACAAAUGUGUCAAUUUCGGAAGCGGCAGGCUGAGACGAGGCAGCGGCGCCACCAACUUCAG |
| | | CCUGUUGAAGCAGGCCGGCGACGUGGAGGAAAACCCCGGCCCCAUGGGAGUCAAAGUUCUGUUUGCCCUGAUCU |
| | | GCAUUGCUGUGGCCGAGGCCAAGAGAGUCCAACCAACAGAAUCUAUUGUUAGAUUUCCUAAUAUUACAAACUUGU |
| | | GCCCUUUUGGUGAAGUUUUUAACGCCACCAGAUUUGCAUCCGUGUAUGCUUGGAACAGGAAGAGAAUCAGCAACU |
| | | GUGUUGCUGAUUAUUCUGUCCUAUAUAAUUCCGCAUCAUUUUCCACUUUUAAGUGUUAUGGAGUGUCUCCUACUA |
| | | AAUUAAAUGAUCUCUGCUUUACUAAUGUCUAUGCAGAUUCAUUUGUAAUUAGAGGUGAUGAAGUCAGACAAAUCG |
| | | CUCCAGGGCAAACUGGAAAGAUUGCUGAUUAUAAUUAUAAAUUACCAGAUGAUUUUACAGGCUGCGUUAUAGCUU |
| | | GGAAUUCUAACAAUCUUGAUUCUAAGGUUGGUGGUAAUUAUAAUUACCUGUAUAGAUUGUUUAGGAAGUCUAAUC |
| | | UCAAACCUUUUGAGAGAGAUAUUUCAACUGAAAUCUAUCAGGCCGGUAGCACACCUUGUAAUGGUGUUGAAGGUU |
| | | UUAAUUGUUACUUUCCUUUACAAUCAUAUGGUUUCCAACCCACUAAUGGUGUUGGUUACCAACCAUACAGAGUAG |
| | | UAGUACUUUCUUUUGAACUUCUACAUGCACCAGCAACUGUUUGUGGACCUAAAAAGUCUACUAAUUUGGUUAAAAA |
| | | CAAAUGUGUCAAUUUC |
| 135 | 2A3461 | AUGGGAGUCAAAGUUCUGUUUGCCCUGAUCUGCAUUGCUGUGGCCGAGGCCAAGAGAGUCCAACCAACAGAAUCU |
| | | AUUGUUAGAUUUCCUAAUAUUACAAACUUGUGCCCUUUUGGUGAAGUUUUUAACGCCACCAGAUUUGCAUCCGUG |
| | | UAUGCUUGGAACAGGAAGAGAAUCAGCAACUGUGUUGCUGAUUAUUCUGUCCUAUAUAAUUCCGCAUCAUUUUCC |
| | | ACUUUUAAGUGUUAUGGAGUGUCUCCUACUAAAUUAAAUGAUCUCUGCUUUACUAAUGUCUAUGCAGAUUCAUUU |
| | | GUAAUUAGAGGUGAUGAAGUCAGACAAAUCGCUCCAGGGCAAACUGGAAAGAUUGCUGAUUAUAAUUAUAAAUUAC |
| | | CAGAUGAUUUUACAGGCUGCGUUAUAGCUUGGAAUUCUAACAAUCUUGAUUCUAAGGUUGGUGGUAAUUAUAAUU |
| | | ACCUGUAUAGAUUGUUUAGGAAGUCUAAUCUCAAACCUUUUGAGAGAGAUAUUUCAACUGAAAUCUAUCAGGCCG |
| | | GUAGCACACCUUGUAAUGGUGUUGAAGGUUUUAAUUGUUACUUUCCUUUACAAUCAUAUGGUUUCCAACCCACUA |
| | | AUGGUGUUGGUUACCAACCAUACAGAGUAGUAGUACUUUCUUUUGAACUUCUACAUGCACCAGCAACUGUUUGUG |
| | | GACCUAAAAAGUCUACUAAUUUGGUUAAAAACAAAUGUGUCAAUUUCGGAAGCGGCAGGCUGAGACGAGGCAGCG |
| | | GCGCCACCAACUUCAGCCUGUUGAAGCAGGCCGGCGACGUGGAGGAAAACCCCGGCCCCAGAGUCCAACCAACAG |
| | | AAUCUAUUGUUAGAUUUCCUAAUAUUACAAACUUGUGCCCUUUUGGUGAAGUUUUUAACGCCACCAGAUUUGCAU |
| | | CCGUGUAUGCUUGGAACAGGAAGAGAAUCAGCAACUGUGUUGCUGAUUAUUCUGUCCUAUAUAAUUCCGCAUCAU |
| | | UUUCCACUUUUAAGUGUUAUGGAGUGUCUCCUACUAAAUUAAAUGAUCUCUGCUUUACUAAUGUCUAUGCAGAUU |
| | | CAUUUGUAAUUAGAGGUGAUGAAGUCAGACAAAUCGCUCCAGGGCAAACUGGAAAGAUUGCUGAUUAUAAUUAUA |
| | | AAUUACCAGAUGAUUUUACAGGCUGCGUUAUAGCUUGGAAUUCUAACAAUCUUGAUUCUAAGGUUGGUGGUAAUU |
| | | AUAAUUACCUGUAUAGAUUGUUUAGGAAGUCUAAUCUCAAACCUUUUGAGAGAGAUAUUUCAACUGAAAUCUAUCA |
| | | GGCCGGUAGCACACCUUGUAAUGGUGUUGAAGGUUUUAAUUGUUACUUUCCUUUACAAUCAUAUGGUUUCCAACC |
| | | CACUAAUGGUGUUGGUUACCAACCAUACAGAGUAGUAGUACUUUUUUUGAACUUCUACAUGCACCAGCAACUGU |
| | | UUGUGGACCUAAAAAGUCUACUAAUUUGGUUAAAAACAAAUGUGUCAAUUUC |
| 136 | 2A4461 | AUGGGAGUCAAAGUUCUGUUUGCCCUGAUCUGCAUUGCUGUGGCCGAGGCCAAGAGAGUCCAACCAACAGAAUCU |
| | | AUUGUUAGAUUUCCUAAUAUUACAAACUUGUGCCCUUUUGGUGAAGUUUUUAACGCCACCAGAUUUGCAUCCGUG |
| | | UAUGCUUGGAACAGGAAGAGAAUCAGCAACUGUGUUGCUGAUUAUUCUGUCCUAUAUAAUUCCGCAUCAUUUUCC |
| | | ACUUUUAAGUGUUAUGGAGUGUCUCCUACUAAAUUAAAUGAUCUCUGCUUUACUAAUGUCUAUGCAGAUUCAUUU |
| | | GUAAUUAGAGGUGAUGAAGUCAGACAAAUCGCUCCAGGGCAAACUGGAAAGAUUGCUGAUUAUAAUUAUAAAUUAC |
| | | CAGAUGAUUUUACAGGCUGCGUUAUAGCUUGGAAUUCUAACAAUCUUGAUUCUAAGGUUGGUGGUAAUUAUAAUU |
| | | ACCUGUAUAGAUUGUUUAGGAAGUCUAAUCUCAAACCUUUUGAGAGAGAUAUUUCAACUGAAAUCUAUCAGGCCG |
| | | GUAGCACACCUUGUAAUGGUGUUGAAGGUUUUAAUUGUUACUUUCCUUUACAAUCAUAUGGUUUCCAACCCACUA |
| | | AUGGUGUUGGUUACCAACCAUACAGAGUAGUAGUACUUUCUUUUGAACUUCUACAUGCACCAGCAACUGUUUGUG |
| | | GACCUAAAAAGUCUACUAAUUUGGUUAAAAACAAAUGUGUCAAUUUCGGAAGCGGCAGGCUGAGACGAGGCAGCG |
| | | GCGCCACCAACUUCAGCCUGUUGAAGCAGGCCGGCGACGUGGAGGAAAACCCCGGCCCCAGAGUCCAACCAACAG |
| | | AAUCUAUUGUUAGAUUUCCUAAUAUUACAAACUUGUGCCCUUUUGGUGAAGUUUUUAACGCCACCAGAUUUGCAU |
| | | CCGUGUAUGCUUGGAACAGGAAGAGAAUCAGCAACUGUGUUGCUGAUUAUUCUGUCCUAUAUAAUUCCGCAUCAU |
| | | UUUCCACUUUUAAGUGUUAUGGAGUGUCUCCUACUAAAUUAAAUGAUCUCUGCUUUACUAAUGUCUAUGCAGAUU |
| | | CAUUUGUAAUUAGAGGUGAUGAAGUCAGACAAAUCGCUCCAGGGCAAACUGGAAAGAUUGCUGAUUAUAAUUAUA |
| | | AAUUACCAGAUGAUUUUACAGGCUGCGUUAUAGCUUGGAAUUCUAACAAUCUUGAUUCUAAGGUUGGUGGUAAUU |
| | | AUAAUUACCUGUAUAGAUUGUUUAGGAAGUCUAAUCUCAAACCUUUUGAGAGAGAUAUUUCAACUGAAAUCUAUCA |
| | | GGCCGGUAGCACACCUUGUAAUGGUGUUGAAGGUUUUAAUUGUUACUUUCCUUUACAAUCAUAUGGUUUCCAACC |
| | | CACUAAUGGUGUUGGUUACCAACCAUACAGAGUAGUAGUACUUUCUUUUGAACUUCUACAUGCACCAGCAACUGU |
| | | UUGUGGACCUAAAAAGUCUACUAAUUUGGUUAAAAACAAAUGUGUCAAUUUCGGAAGCGGCAGGCUGAGACGAGG |
| | | CAGCGGCGCCACCAACUUCAGCCUGUUGAAGCAGGCCGGCGACGUGGAGGAAAACCCCGGCCCCAGAGUCCAAC |
| | | CAACAGAAUCUAUUGUUAGAUUUCCUAAUAUUACAAACUUGUGCCCUUUUGGUGAAGUUUUUAACGCCACCAGAU |
| | | UUGCAUCCGUGUAUGCUUGGAACAGGAAGAGAAUCAGCAACUGUGUUGCUGAUUAUUCUGUCCUAUAUAAUUCCG |
| | | CAUCAUUUUCCACUUUUAAGUGUUAUGGAGUGUCUCCUACUAAAUUAAAUGAUCUCUGCUUUACUAAUGUCUAUG |
| | | CAGAUUCAUUUGUAAUUAGAGGUGAUGAAGUCAGACAAAUCGCUCCAGGGCAAACUGGAAAGAUUGCUGAUUAUA |
| | | AUUAUAAAUUACCAGAUGAUUUUACAGGCUGCGUUAUAGCUUGGAAUUCUAACAAUCUUGAUUCUAAGGUUGGUG |
| | | GUAAUUAUAAUUACCUGUAUAGAUUGUUUAGGAAGUCUAAUCUCAAACCUUUUGAGAGAGAUAUUUCAACUGAAAU |
| | | CUAUCAGGCCGGUAGCACACCUUGUAAUGGUGUUGAAGGUUUUAAUUGUUACUUUCCUUUACAAUCAUAUGGUUU |
| | | CCAACCCACUAAUGGUGUUGGUUACCAACCAUACAGAGUAGUAGUACUUUCUUUUGAACUUCUACAUGCACCAGC |
| | | AACUGUUUGUGGACCUAAAAAGUCUACUAAUUUGGUUAAAAACAAAUGUGUCAAUUUC |
| 137 | 2A5461 | AUGGGAGUCAAAGUUCUGUUUGCCCUGAUCUGCAUUGCUGUGGCCGAGGCCAAGAGAGUCCAACCAACAGAAUCU |
| | | AUUGUUAGAUUUCCUAAUAUUACAAACUUGUGCCCUUUUGGUGAAGUUUUUAACGCCACCAGAUUUGCAUCCGUG |
| | | UAUGCUUGGAACAGGAAGAGAAUCAGCAACUGUGUUGCUGAUUAUUCUGUCCUAUAUAAUUCCGCAUCAUUUUCC |
| | | ACUUUUAAGUGUUAUGGAGUGUCUCCUACUAAAUUAAAUGAUCUCUGCUUUACUAAUGUCUAUGCAGAUUCAUUU |
| | | GUAAUUAGAGGUGAUGAAGUCAGACAAAUCGCUCCAGGGCAAACUGGAAAGAUUGCUGAUUAUAAUUAUAAAUUAC |
| | | CAGAUGAUUUUACAGGCUGCGUUAUAGCUUGGAAUUCUAACAAUCUUGAUUCUAAGGUUGGUGGUAAUUAUAAUU |
| | | ACCUGUAUAGAUUGUUUAGGAAGUCUAAUCUCAAACCUUUUGAGAGAGAUAUUUCAACUGAAAUCUAUCAGGCCG |
| | | GUAGCACACCUUGUAAUGGUGUUGAAGGUUUUAAUUGUUACUUUCCUUUACAAUCAUAUGGUUUCCAACCCACUA |
| | | AUGGUGUUGGUUACCAACCAUACAGAGUAGUAGUACUUUCUUUUGAACUUCUACAUGCACCAGCAACUGUUUGUG |
| | | GACCUAAAAAGUCUACUAAUUUGGUUAAAAACAAAUGUGUCAAUUUCGGAAGCGGCAGGCUGAGACGAGGCAGCG |
| | | GCGCCACCAACUUCAGCCUGUUGAAGCAGGCCGGCGACGUGGAGGAAAACCCCGGCCCCGGCCUCGAACAACUC |
| | | GAAAGCAUUAUCAAUUUUGAAAAACUCACAGAAUGGACCAGUGGACUCGAGCAACUCGAGAGUAUCAUUAAUUUCG |
| | | AAAAGCUCACAGAGUGGACAAGCGGCCUGGAGCAGCUGGAGAGCAUCAUAAACUUCGAGAAGCUGACCGAGUGGA |
| | | CCAGC |
| 138 | 2A6461 | AUGGGAGUCAAAGUUCUGUUUGCCCUGAUCUGCAUUGCUGUGGCCGAGGCCAAGAGAGUCCAACCAACAGAAUCU |
| | | AUUGUUAGAUUUCCUAAUAUUACAAACUUGUGCCCUUUUGGUGAAGUUUUUAACGCCACCAGAUUUGCAUCCGUG |
| | | UAUGCUUGGAACAGGAAGAGAAUCAGCAACUGUGUUGCUGAUUAUUCUGUCCUAUAUAAUUCCGCAUCAUUUUCC |
| | | ACUUUUAAGUGUUAUGGAGUGUCUCCUACUAAAUUAAAUGAUCUCUGCUUUACUAAUGUCUAUGCAGAUUCAUUU |
| | | GUAAUUAGAGGUGAUGAAGUCAGACAAAUCGCUCCAGGGCAAACUGGAAAGAUUGCUGAUUAUAAUUAUAAAUUAC |
| | | CAGAUGAUUUUACAGGCUGCGUUAUAGCUUGGAAUUCUAACAAUCUUGAUUCUAAGGUUGGUGGUAAUUAUAAUU |
| | | ACCUGUAUAGAUUGUUUAGGAAGUCUAAUCUCAAACCUUUUGAGAGAGAUAUUUCAACUGAAAUCUAUCAGGCCG |
| | | GUAGCACACCUUGUAAUGGUGUUGAAGGUUUUAAUUGUUACUUUCCUUUACAAUCAUAUGGUUUCCAACCCACUA |
| | | AUGGUGUUGGUUACCAACCAUACAGAGUAGUAGUACUUUCUUUUGAACUUCUACAUGCACCAGCAACUGUUUGUG |
| | | GACCUAAAAAGUCUACUAAUUUGGUUAAAAACAAAUGUGUCAAUUUCCGGCGGAAGCGGAGAAGCGGCAGCGGCG |
| | | CCACCAACUUCAGCCUGUUGAAGCAGGCCGGCGACGUGGAGGAAAACCCCGGCCCCAUGGGAGUCAAAGUUCUG |
| | | UUUGCCCUGAUCUGCAUUGCUGUGGCCGAGGCCAAGAGAGUCCAACCAACAGAAUCUAUUGUUAGAUUUCCUAAU |
| | | AUUACAAACUUGUGCCCUUUUGGUGAAGUUUUUAACGCCACCAGAUUUGCAUCCGUGUAUGCUUGGAACAGGAAG |
| | | AGAAUCAGCAACUGUGUUGCUGAUUAUUCUGUCCUAUAUAAUUCCGCAUCAUUUUCCACUUUUAAGUGUUAUGGA |
| | | GUGUCUCCUACUAAAUUAAAUGAUCUCUGCUUUACUAAUGUCUAUGCAGAUUCAUUUGUAAUUAGAGGUGAUGAA |
| | | GUCAGACAAAUCGCUCCAGGGCAAACUGGAAAGAUUGCUGAUUAUAAUUAUAAAUUACCAGAUGAUUUUACAGGCU |
| | | GCGUUAUAGCUUGGAAUUCUAACAAUCUUGAUUCUAAGGUUGGUGGUAAUUAUAAUUACCUGUAUAGAUUGUUUA |
| | | GGAAGUCUAAUCUCAAACCUUUUGAGAGAGAUAUUUCAACUGAAAUCUAUCAGGCCGGUAGCACACCUUGUAAUG |
| | | GUGUUGAAGGUUUUAAUUGUUACUUUCCUUUACAAUCAUAUGGUUUCCAACCCACUAAUGGUGUUGGUUACCAAC |
| | | CAUACAGAGUAGUAGUACUUUCUUUUGAACUUCUACAUGCACCAGCAACUGUUUGUGGACCUAAAAAGUCUACUAA |
| | | UUUGGUUAAAAACAAAUGUGUCAAUUUC |
| 139 | 2A6481 | AUAGGUCUCACAUGUUUGUUUUCUUAGUUUUAUUACCUUUAGUUUCUUCCCAGUGUGUUAAUUUAACUACUAGAA |
| | | CUCAAUUACCUCCUGCUUAUACUAAUUCUUUUACUAGAGGAGUGUAUUAUCCUGAUAAGGUUUUUAGAUCUUCUG |
| | | UUCUGCACUCUACUCAGGAUUUGUUCUUGCCCUUCUUCUCUAACGUGACCUGGUUCCACGCUAUUCACGUUUCU |
| | | GGAACCAACGGCACUAAGAGAUUUGAUAAUCCCGUUUUGCCUUUUAACGACGGAGUUUAUUUCGCCUCUACCGAA |
| | | AAAUCUAACAUCAUUAGGGGCUGGAUCUUUGGAACUACCCUGGAUUCUAAAACUCAAUCUUUAUUGAUUGUGAAU |
| | | AACGCUACCAACGUUGUGAUUAAAGUUUGUGAAUUUCAAUUUUGUAACGAUCCUUUUCUGGGAGUUUAUUAUCAU |
| | | AAAAACAAUAAAUCUUGGAUGGAAUCUGAAUUCCGCGUUUAUUCUUCUGCCAAUAACUGUACCUUUGAGUACGUU |
| | | UCUCAACCUUUCUUAAUGGAUUUAGAAGGAAAACAAGGCAAUUUCAAAAACUUAAGAGAAUUUGUUUUUAAAAAUA |
| | | UUGACGGAUAUUUUAAAAUUUAUUCUAAGCAUACUCCUAUUAAUUUAGUGAGAGACUUACCCCAAGGAUUUUCUGC |
| | | UUUAGAACCUUUAGUUGACUUACCUAUUGGAAUCAAUAUCACUAGAUUUCAAACUUUAUUAGCUUUACAUAGAUCU |
| | | UAUUUAACUCCUGGAGAUUCUUCCUCCGGCUGGACAGCCGGAGCCGCUGCUUAUUACGUUGGAUAUUUACAGCC |
| | | UAGAACUUUUCUGCUGAAAUAUAACGAAAACGGAACCAUUACCGACGCCGUUGACUGUGCUCUGGAUCCUUUAUC |
| | | UGAAACCAAGUGUACUUUAAAAUCUUUUACCGUUGAAAAAGGAAUUUAUCAAACUUCUAAUUUUAGAGUUCAACCU |
| | | ACUGAAUCUAUCGUUAGAUUCCCUAAUAUUACUAAUUUGUGUCCCUUUGGAGAGGUUUUCAACGCUACUAGAUUU |
| | | GCCUCUGUUUACGCUUGGAAUCGCAAGAGAAUUUCUAAUUGUGUGGCCGAUUACUCCGUUUUGUAUAACUCUGCU |
| | | UCUUUCUCUACUUUUAAGUGUUACGGAGUUUCCCCUACUAAACUGAACGAUUUGUGUUUUACUAACGUUUACGCU |
| | | GAUUCUUUUGUUAUUAGAGGCGACGAAGUUAGACAAAUUGCUCCUGGACAAACUGGAAAGAUUGCUGACUAUAAC |
| | | UAUAAACUGCCUGACGAUUUUACCGGCUGUGUUAUUGCUUGGAAUUCUAAUAAUUUAGAUUCUAAAGUUGGAGGA |
| | | AAUUAUAAUUAUUUAUAUAGAUUAUUUAGAAAAUCUAAUUUAAAACCUUUUGAAAGAGAUAUUUCCACUGAGAUUUA |
| | | CCAGGCUGGAUCUACUCCUUGUAACGGCGUUGAGGGAUUUAAUUGUUAUUUUCCUUUACAAUCUUACGGAUUUCA |
| | | ACCUACCAACGGAGUUGGAUAUCAACCUUACAGAGUUGUGGUGCUGUCUUUUGAAUUAUUACACGCUCCCGCUAC |
| | | UGUUUGCGGCCCUAAAAAGUCUACUAAUCUGGUUAAGAAUAAGUGUGUGAAUUUUAAUUUCAACGGAUUGACCGG |
| | | AACUGGAGUUCUGACUGAAUCUAAUAAAAAAUUUUUACCCUUUCAACAAUUUGGAAGGGAUAUCGCUGAUACUACU |
| | | GACGCUGUUAGAGAUCCUCAAACUUUAGAAAUCCUGGAUAUUACUCCUUGUUCUUUUGGAGGAGUUUCCGUUAUU |
| | | ACUCCUGGCACUAAUACUUCUAAUCAAGUUGCUGUUUUAUAUCAAGACGUUAAUUGCACCGAAGUUCCUGUUGCU |
| | | AUUCACGCUGAUCAGUUAACUCCUACUUGGAGAGUUUAUUCUACUGGAUCUAACGUUUUUCAAACCAGAGCUGGC |
| | | UGUUUAAUCGGAGCUGAACACGUGAAUAAUUCUUACGAGUGCGAUAUUCCCAUCGGAGCUGGAAUUUGUGCCUCU |
| | | UAUCAAACUCAAACCAAUUCUCCUAGGAGGGCUAGAUCCGUUGCCUCUCAAUCUAUUAUUGCUUAUACCAUGUCU |
| | | UUAGGAGCUGAAAAUUCCGUUGCUUACUCUAACAAUUCUAUUGCUAUUCCUACUAACUUUACUAUUUCUGUUACUA |
| | | CUGAAAUUCUGCCCGUGUCCAUGACUAAAACUUCUGUUGAUUGUACUAUGUAUAUUUGUGGAGAUUCCACUGAGU |
| | | GUUCUAACCUGUUAUUACAAUACGGAUCUUUUUGUACUCAAUUAAAUAGAGCUCUGACUGGAAUUGCUGUUGAAC |
| | | AGGACAAAAAUACUCAAGAAGUUUUUGCCCAAGUUAAACAGAUUUAUAAAACUCCUCCUAUUAAAGAUUUUGGAGG |
| | | AUUUAAUUUCUCUCAAAUUUUACCUGACCCUUCUAAGCCUUCCAAAAGGUCUUUCAUCGAAGAUUUAUUAUUUAAU |
| | | AAAGUUACCUUAGCCGACGCUGGAUUUAUUAAGCAAUACGGAGAUUGCUUAGGCGAUAUCGCUGCUAGAGACUUA |
| | | AUUUGUGCUCAAAAAUUUAACGGAUUAACUGUUUUACCUCCCCUGUUAACUGACGAAAUGAUCGCUCAAUAUACUU |
| | | CUGCUCUGUUAGCUGGAACUAUUACUUCUGGCUGGACUUUUGGAGCUGGAGCUGCUUUACAAAUUCCCUUUGCU |
| | | AUGCAGAUGGCUUAUAGAUUUAACGGAAUUGGAGUUACUCAAAACGUUUUAUACGAAAAUCAAAAAUUAAUUGCUA |
| | | AUCAAUUUAAUUCUGCUAUUGGAAAAAUUCAAGAUUCCCUGUCUUCUACUGCUUCCGCUCUGGGAAAAUUACAAGA |
| | | CGUUGUUAAUCAAAACGCUCAAGCUUUGAAUACCUUAGUUAAACAAUUAUCUUCCAAUUUUGGAGCUAUUUCUUCU |
| | | GUUUUAAACGACAUUUUAUCUAGAUUAGAUCCCCCUGAAGCUGAAGUUCAGAUUGACAGAUUGAUUACCGGAAGA |
| | | UUACAAUCUUUACAAACUUACGUUACUCAACAACUGAUUAGAGCUGCUGAAAUUAGAGCCUCUGCUAAUUUAGCUG |
| | | CUACUAAGAUGUCUGAGUGUGUUUUAGGACAGUCCAAAAGAGUUGAUUUUUGUGGAAAAGGCUAUCAUCUGAUGU |
| | | CUUUUCCUCAAUCUGCUCCUCACGGAGUUGUGUUUUUACACGUUACUUACGUUCCUGCUCAAGAGAAGAAUUUUA |
| | | CUACUGCUCCUGCUAUUUGUCACGACGGAAAAGCUCAUUUUCCUAGAGAAGGAGUUUUUGUUUCUAACGGAACUC |
| | | AUUGGUUUGUUACUCAAAGAAACUUUUACGAACCUCAGAUUAUUACCACUGAUAAUACCUUUGUUUCUGGCAAUU |
| | | GUGACGUGGUUAUUGGAAUUGUUAAUAAUACUGUUUACGAUCCUUUACAACCCGAACUGGAUUCUUUUAAAGAAG |
| | | AAUUAGAUAAAUACUUUAAAAAUCAUACUUCUCCCGACGUUGAUUUAGGAGAUAUCUCUGGAAUUAACGCUUCUGU |
| | | UGUGAAUAUUCAAAAGGAAAUUGAUAGAUUAAACGAAGUGGCUAAAAAUUUAAACGAGUCUUUAAUUGAUUUACAG |
| | | GAACUGGGAAAAUACGAACAAUAUAUUAAGUGGCCUUGGUACAUUUGGUUAGGCUUUAUUGCUGGACUGAUUGCU |
| | | AUUGUGAUGGUUACUAUCAUGUUGUGUUGCAUGACUUCUUGUUGUUCCUGUUUAAAAGGCUGUUGCUCUUGUGG |
| | | AUCUUGUUGCAAAUUUGACGAAGACGAUUCUGAACCUGUUCUGAAAGGAGUUAAACUGCAUUAUACUUAAAUGAG |
| | | ACCAUA |
| 140 | 2A7481 | AUAGGUCUCACAUGUUCGUGUUCUUAGUGCUGUUACCCCUGGUUUCUUCUCAGUGUGUCAAUUUAACCACCAGAA |
| | | CCCAACUGCCUCCUGCCUAUACUAAUUCCUUUACCCGCGGAGUUUACUAUCCCGAUAAGGUUUUUCGCUCCUCUG |
| | | UUUUACACUCUACUCAGGACUUAUUCUUACCCUUCUUCUCCAACGUUACUUGGUUUCACGCCAUUCACGUUUCUG |
| | | GAACCAACGGAACUAAGAGAUUCGAUAAUCCUGUGUUACCUUUCAACGACGGAGUUUAUUUUGCUUCCACUGAAA |
| | | AAUCCAAUAUCAUUAGGGGCUGGAUCUUUGGAACCACUUUAGAUUCCAAGACUCAAUCUUUAUUAAUUGUGAAUAA |
| | | CGCCACCAACGUGGUUAUCAAAGUGUGUGAAUUCCAAUUUUGUAACGACCCUUUCUUAGGCGUGUAUUAUCACAA |
| | | AAAUAAUAAAUCUUGGAUGGAAUCUGAAUUUCGCGUUUACUCUUCCGCUAACAACUGCACUUUUGAGUACGUUUC |
| | | CCAGCCUUUUCUGAUGGAUCUGGAAGGCAAACAGGGAAAUUUUAAAAAUCUGAGAGAAUUUGUUUUUAAGAACAU |
| | | UGACGGAUAUUUCAAGAUCUAUUCUAAACACACUCCCAUCAAUUUGGUUAGAGAUUUACCUCAGGGCUUUUCUGC |
| | | UUUAGAACCUUUAGUUGACCUGCCUAUUGGAAUCAACAUUACUAGAUUUCAAACUCUGUUAGCUCUGCAUAGAUC |
| | | UUAUUUAACUCCUGGAGAUUCCUCCUCCGGCUGGACCGCCGGAGCUGCUGCUUAUUACGUUGGCUAUUUACAAC |
| | | CCAGAACUUUUCUGUUAAAAUAUAACGAAAACGGCACCAUCACUGACGCCGUGGAUUGUGCUUUAGAUCCCUUAU |
| | | CCGAAACUAAGUGUACUCUGAAAUCCUUCACUGUUGAAAAAGGAAUUUAUCAGACCUCUAAUUUUAGAGUGCAACC |
| | | UACCGAAUCUAUUGUUAGAUUUCCUAAUAUUACCAACUUGUGCCCUUUUGGCGAAGUUUUCAACGCUACUAGAUU |
| | | UGCUUCUGUUUACGCUUGGAAUAGAAAAAGAAUUUCUAACUGUGUGGCUGACUAUUCCGUGUUAUACAAUUCUGC |
| | | CUCUUUUUCUACUUUUAAGUGUUACGGAGUGUCUCCCACUAAACUGAACGAUUUGUGCUUUACCAACGUUUACGC |
| | | UGAUUCUUUUGUUAUUAGAGGCGACGAGGUUCGCCAGAUUGCCCCUGGCCAGACUGGCAAAAUUGCUGACUAUAA |
| | | CUAUAAAUUACCUGACGAUUUUACCGGCUGCGUUAUCGCUUGGAACUCUAAUAAUCUGGAUUCUAAAGUUGGAGG |
| | | AAACUACAAUUAUUUAUAUAGAUUAUUUAGAAAAUCUAAUUUAAAACCCUUCGAGAGGGAUAUCUCUACUGAAAUC |
| | | UACCAAGCUGGAUCUACUCCUUGCAACGGCGUUGAAGGAUUUAAUUGUUAUUUUCCUUUACAAUCUUACGGAUUC |
| | | CAACCUACUAACGGAGUUGGAUAUCAGCCUUAUAGAGUGGUGGUUUUAUCUUUCGAAUUAUUACACGCUCCCGCU |
| | | ACUGUGUGUGGACCCAAAAAGUCUACUAAUUUAGUUAAAAAUAAGUGUGUUAAUUUCAAUUUUAACGGACUCACUG |
| | | GCACUGGAGUGUUAACUGAAUCUAAUAAAAAGUUCUUACCUUUCCAACAGUUCGGACGCGACAUUGCCGACACCA |
| | | CUGACGCUGUGAGAGAUCCUCAAACCUUAGAGAUUCUGGAUAUUACCCCUUGUUCCUUUGGAGGCGUUUCCGUU |
| | | AUCACUCCCGGAACUAACACUUCUAAUCAGGUUGCCGUUUUAUAUCAGGACGUUAAUUGCACUGAAGUUCCUGUU |
| | | GCUAUUCACGCUGAUCAACUGACUCCUACUUGGAGAGUUUAUUCCACUGGAUCUAACGUUUUUCAGACUCGCGCC |
| | | GGCUGUUUAAUUGGAGCUGAACACGUGAACAACUCCUACGAGUGCGAUAUUCCUAUUGGCGCUGGCAUCUGUGC |
| | | UUCUUAUCAAACUCAAACUAAUUCUCCCAGGAGGGCUCGCUCUGUUGCUUCCCAAUCUAUCAUUGCUUAUACUAU |
| | | GUCCUUAGGCGCUGAAAAUUCUGUUGCUUAUUCCAACAAUUCUAUUGCUAUUCCCACCAAUUUUACUAUUUCUGU |
| | | GACUACUGAAAUUUUACCUGUUUCCAUGACUAAGACUUCUGUUGAUUGUACUAUGUAUAUCUGCGGAGAUUCUAC |
| | | UGAGUGUUCCAACCUCUUAUUACAGUACGGAUCUUUUUGCACUCAGUUAAAUAGAGCUCUGACUGGAAUCGCUGU |
| | | UGAACAAGACAAAAACACCCAAGAAGUGUUUGCUCAGGUGAAGCAAAUUUACAAGACACCUCCUAUUAAGGAUUUU |
| | | GGAGGAUUCAAUUUUUCCCAGAUUUUACCUGAUCCUUCUAAACCUUCUAAACGCUCCUUUAUUGAAGAUUUACUG |
| | | UUCAAUAAAGUGACUCUGGCUGACGCUGGCUUCAUCAAGCAAUACGGCGAUUGUUUAGGAGACAUCGCUGCUAGA |
| | | GACUUAAUUUGUGCUCAAAAAUUCAACGGACUGACUGUUCUGCCCCCUUUACUGACCGACGAGAUGAUCGCCCAA |
| | | UAUACUUCUGCCCUGUUAGCUGGCACCAUUACUUCUGGCUGGACCUUUGGAGCUGGCGCUGCUUUACAAAUUCC |
| | | UUUCGCCAUGCAGAUGGCUUAUCGCUUUAACGGAAUUGGCGUUACCCAAAACGUGUUAUACGAGAAUCAAAAAUU |
| | | AAUCGCCAAUCAAUUUAAUUCUGCUAUUGGCAAGAUUCAAGAUUCUUUAUCUUCUACUGCUUCCGCCCUGGGCAA |
| | | ACUGCAGGACGUUGUUAACCAGAACGCUCAAGCCCUGAACACUUUAGUGAAGCAACUGUCUUCUAACUUCGGCGC |
| | | UAUUUCUUCCGUUUUAAACGACAUUCUGUCUCGCUUAGAUCCCCCUGAGGCUGAGGUUCAAAUUGAUAGACUGAU |
| | | UACUGGAAGAUUACAGUCCCUGCAAACUUACGUUACUCAACAGUUAAUCAGAGCUGCUGAAAUCCGCGCUUCCGC |
| | | UAAUUUAGCUGCUACUAAAAUGUCUGAGUGUGUGUUAGGCCAGUCUAAGAGAGUUGACUUUUGUGGCAAGGGCU |
| | | AUCAUUUAAUGUCUUUUCCUCAGUCCGCUCCUCACGGCGUUGUGUUCUUACACGUGACUUACGUUCCCGCCCAAG |
| | | AGAAAAAUUUUACCACUGCUCCUGCUAUUUGUCACGACGGAAAAGCUCAUUUUCCUAGAGAAGGAGUGUUUGUUU |
| | | CUAACGGAACCCACUGGUUUGUGACUCAAAGAAACUUUUACGAACCCCAAAUUAUUACUACUGACAAUACUUUCGU |
| | | UUCCGGCAACUGUGACGUGGUGAUCGGAAUUGUUAAUAACACCGUUUACGAUCCCUUACAACCUGAAUUAGAUUC |
| | | UUUUAAAGAAGAAUUAGAUAAAUAUUUCAAAAAUCAUACUUCCCCUGACGUUGAUCUGGGAGAUAUUUCUGGAAUU |
| | | AACGCUUCUGUGGUGAACAUUCAAAAAGAGAUUGACAGAUUAAACGAAGUUGCUAAAAACCUGAACGAGUCCCUGA |
| | | UCGACCUGCAGGAACUGGGAAAAUACGAGCAGUAUAUUAAGUGGCCUUGGUAUAUUUGGUUAGGAUUUAUUGCUG |
| | | GAUUAAUUGCUAUUGUGAUGGUGACCAUUAUGUUGUGCUGUAUGACCUCUUGUUGUUCUUGCCUGAAGGGCUGC |
| | | UGUUCUUGUGGCUCUUGUUGCAAGUUUGACGAAGACGAUUCCGAACCCGUUUUAAAGGGAGUGAAACUGCAUUAU |
| | | ACUUAAAUGAGACCAUA |
| 141 | 2A8481 | AUAGGUCUCACAUGUUUGUUUUUCUGGUUUUAUUACCCUUAGUGUCCUCCCAGUGCGUGAACUUAACUACCCGCA |
| | | CUCAGCUGCCUCCUGCCUAUACCAACUCUUUCACUAGAGGCGUUUACUAUCCUGACAAAGUUUUUAGAUCUUCUG |
| | | UGCUGCAUUCCACUCAAGAUUUAUUCCUGCCCUUCUUUUCUAACGUUACUUGGUUUCACGCCAUCCACGUGUCUG |
| | | GCACCAACGGAACCAAACGCUUUGACAACCCUGUGUUACCUUUUAACGACGGAGUUUACUUCGCCUCCACUGAAA |
| | | AGUCCAAUAUUAUCCGGGGCUGGAUUUUCGGAACCACCUUAGAUUCUAAAACCCAGUCUCUGCUGAUUGUGAACA |
| | | ACGCCACUAACGUGGUUAUUAAAGUGUGCGAGUUUCAAUUUUGCAACGACCCUUUUCUGGGAGUUUAUUACCACA |
| | | AAAAUAACAAAUCUUGGAUGGAGUCUGAAUUUAGAGUGUAUUCUUCCGCCAAUAAUUGCACCUUCGAGUACGUUU |
| | | CCCAACCCUUUCUGAUGGACUUAGAAGGAAAGCAAGGCAAUUUCAAAAACCUGCGCGAAUUUGUGUUCAAAAAUAU |
| | | UGACGGCUAUUUCAAAAUUUACUCCAAGCAUACCCCUAUUAAUUUAGUGCGCGAUCUGCCUCAGGGCUUCUCUGC |
| | | CUUAGAGCCUUUAGUGGACCUGCCUAUUGGCAUUAAUAUUACUCGCUUUCAGACUUUACUGGCCCUGCAUAGAUC |
| | | UUAUUUAACUCCUGGAGACUCUUCUUCCGGCUGGACCGCUGGAGCCGCUGCCUACUACGUUGGCUAUCUGCAGC |
| | | CUAGAACUUUUUUAUUAAAGUACAACGAAAACGGAACCAUCACUGACGCUGUUGAUUGCGCCCUGGAUCCUUUAU |
| | | CUGAAACAAAGUGCACCCUGAAGUCCUUCACCGUUGAAAAGGGCAUUUACCAAACAUCCAAUUUUAGGGUUCAGC |
| | | CUACUGAAUCCAUCGUGCGCUUUCCCAACAUCACUAACUUGUGUCCCUUUGGCGAGGUGUUUAACGCCACUCGCU |
| | | UUGCCUCUGUGUACGCCUGGAACAGAAAGAGAAUUUCCAAUUGCGUGGCUGAUUAUUCUGUGCUGUAUAACUCCG |
| | | CUUCUUUCUCCACCUUUAAGUGUUACGGAGUGUCCCCUACUAAACUGAACGACUUGUGCUUCACCAACGUUUACG |
| | | CCGAUUCUUUUGUGAUUCGCGGCGACGAAGUUAGACAAAUCGCCCCCGGACAGACCGGAAAGAUCGCUGAUUACA |
| | | ACUAUAAGUUACCUGACGACUUCACCGGCUGCGUGAUUGCUUGGAAUUCUAAUAACUUAGACUCUAAAGUUGGAG |
| | | GCAACUACAAUUACCUGUAUAGAUUAUUCCGCAAAUCUAACCUGAAACCUUUUGAGAGAGACAUCUCCACCGAAAU |
| | | UUACCAAGCCGGCUCUACCCCCUGCAACGGAGUUGAAGGAUUUAAUUGCUACUUUCCCCUGCAGUCCUACGGAUU |
| | | CCAACCUACUAACGGCGUUGGAUACCAACCUUACAGAGUUGUGGUGCUGUCUUUUGAAUUAUUACACGCCCCUGC |
| | | CACCGUUUGUGGCCCUAAGAAAUCUACCAAUCUGGUUAAAAACAAGUGUGUGAAUUUUAAUUUUAACGGCCUGAC |
| | | CGGCACUGGCGUUUUAACCGAGUCUAACAAGAAGUUUCUGCCUUUCCAACAAUUUGGCAGAGACAUCGCCGACAC |
| | | CACCGACGCUGUGCGCGAUCCUCAAACCUUAGAAAUUUUAGAUAUUACUCCUUGCUCUUUCGGCGGCGUUUCCGU |
| | | UAUUACUCCUGGAACCAAUACCUCUAAUCAAGUGGCUGUGCUGUACCAGGACGUUAAUUGCACUGAAGUGCCUGU |
| | | GGCUAUUCACGCCGAUCAACUGACCCCCACUUGGAGAGUUUAUUCUACUGGAUCUAACGUGUUCCAAACUAGAGC |
| | | UGGCUGUCUGAUCGGCGCCGAGCACGUUAAUAAUUCUUACGAGUGUGAUAUCCCUAUUGGCGCUGGCAUUUGCG |
| | | CCUCUUACCAGACCCAGACCAACUCCCCCCGCAGAGCUAGAUCCGUGGCCUCUCAGUCCAUUAUUGCCUAUACUA |
| | | UGUCCUUAGGAGCCGAAAAUUCUGUGGCUUACUCCAACAAUUCUAUUGCCAUUCCCACUAAUUUUACUAUCUCUG |
| | | UGACUACCGAAAUCCUGCCCGUGUCCAUGACUAAGACCUCCGUGGACUGCACCAUGUAUAUCUGCGGAGACUCUA |
| | | CUGAGUGUUCCAACUUAUUACUGCAGUACGGCUCUUUCUGUACUCAGCUGAAUAGAGCCCUGACUGGAAUUGCUG |
| | | UGGAGCAAGACAAAAAUACCCAGGAGGUGUUCGCUCAGGUGAAACAAAUUUAUAAGACCCCUCCCAUCAAAGAUUU |
| | | UGGAGGCUUCAAUUUUUCUCAAAUUCUGCCCGACCCCUCCAAGCCUUCCAAAAGAUCCUUUAUUGAAGAUCUGCU |
| | | GUUCAAUAAGGUUACUUUAGCCGACGCCGGCUUCAUUAAGCAGUACGGAGAUUGUCUGGGCGAUAUUGCUGCCC |
| | | GCGACCUGAUUUGCGCUCAGAAGUUCAACGGAUUAACCGUUCUGCCUCCUCUGCUGACUGACGAGAUGAUUGCU |
| | | CAGUAUACUUCUGCCCUGCUGGCUGGAACUAUUACCUCUGGCUGGACCUUCGGCGCCGGAGCUGCUCUGCAAAU |
| | | UCCCUUCGCCAUGCAAAUGGCUUACAGAUUUAACGGCAUCGGAGUUACUCAAAACGUGCUGUACGAAAACCAAAAA |
| | | UUAAUUGCCAACCAGUUCAAUUCCGCUAUCGGCAAAAUUCAGGAUUCUCUGUCUUCUACUGCUUCCGCUUUAGGC |
| | | AAACUGCAAGACGUUGUUAACCAGAACGCCCAAGCUUUAAACACCCUGGUUAAGCAGCUGUCCUCUAAUUUUGGC |
| | | GCUAUCUCUUCUGUUCUGAACGACAUUUUAUCUAGACUGGACCCCCCUGAAGCCGAGGUGCAGAUUGAUAGAUUA |
| | | AUCACCGGAAGACUGCAAUCCCUGCAGACCUACGUUACCCAGCAAUUAAUCAGAGCCGCCGAGAUCAGAGCCUCU |
| | | GCCAACUUAGCUGCCACCAAAAUGUCCGAGUGUGUGCUGGGACAGUCCAAGCGCGUGGACUUCUGUGGAAAGGG |
| | | AUACCACCUGAUGUCUUUUCCUCAAUCUGCUCCCCACGGAGUUGUGUUUCUGCACGUUACUUACGUGCCUGCCCA |
| | | AGAGAAAAACUUUACUACCGCCCCCGCUAUUUGCCACGACGGCAAGGCUCACUUCCCUAGAGAAGGAGUUUUUGU |
| | | GUCUAACGGAACCCAUUGGUUUGUGACUCAAAGAAAUUUUUACGAGCCUCAAAUCAUUACCACCGAUAACACUUUU |
| | | GUUUCCGGCAAUUGUGACGUGGUGAUUGGAAUCGUUAACAACACCGUUUACGACCCUUUACAGCCCGAAUUAGAC |
| | | UCUUUUAAAGAGGAGUUAGAUAAAUAUUUUAAAAACCAUACCUCUCCCGACGUGGACCUGGGAGAUAUUUCCGGA |
| | | AUUAACGCUUCUGUUGUGAAUAUCCAGAAAGAGAUCGACAGAUUAAACGAGGUUGCUAAAAAUUUAAACGAAUCCC |
| | | UGAUUGACCUGCAGGAGCUGGGCAAAUACGAACAAUAUAUUAAGUGGCCUUGGUAUAUUUGGUUAGGCUUCAUCG |
| | | CUGGAUUAAUCGCUAUUGUUAUGGUGACUAUUAUGUUGUGUUGUAUGACUUCCUGCUGUUCUUGCUUAAAAGGC |
| | | UGCUGUUCCUGCGGAUCCUGUUGUAAAUUCGACGAGGACGAUUCCGAGCCUGUUUUAAAAGGAGUGAAAUUACAU |
| | | UAUACUUAAAUGAGACCAUA |
| 142 | 2A9481 | AUAGGUCUCACAUGUUCGUGUUCCUGGUGCUGCUGCCCUUAGUGUCCUCCCAGUGUGUGAACCUGACCACCAGA |
| | | ACCCAGUUACCUCCCGCUUACACCAACUCCUUUACCCGCGGAGUGUACUACCCUGAUAAGGUUUUUAGAUCCUCC |
| | | GUUCUGCAUUCUACCCAAGACCUGUUUUUACCCUUCUUUUCCAACGUGACCUGGUUCCACGCUAUCCACGUGUCC |
| | | GGCACUAACGGCACUAAGCGCUUCGACAAUCCUGUGCUCCCUUUUAACGACGGCGUGUACUUCGCUUCUACCGAA |
| | | AAAUCCAACAUCAUCCGCGGCUGGAUCUUCGGAACUACCCUGGAUUCUAAAACCCAGUCUUUAUUAAUCGUGAAU |
| | | AACGCCACCAACGUGGUUAUUAAGGUCUGUGAGUUCCAAUUUUGCAACGACCCUUUCUUAGGCGUUUAUUACCAC |
| | | AAAAACAAUAAGUCUUGGAUGGAGUCCGAAUUCCGCGUGUACUCCUCUGCCAACAAUUGCACCUUUGAGUACGUG |
| | | UCCCAGCCCUUCCUGAUGGACCUGGAAGGCAAGCAGGGCAAUUUUAAAAAUCUGCGCGAAUUCGUUUUCAAGAAC |
| | | AUCGACGGCUACUUUAAGAUUUAUUCCAAACAUACCCCCAUCAACCUGGUGCGCGAUUUACCCCAGGGCUUCUCC |
| | | GCCUUAGAGCCCUUAGUGGAUCUGCCUAUCGGCAUCAAUAUCACCCGCUUUCAGACUCUGCUGGCCCUGCACAGA |
| | | UCCUACCUGACUCCCGGAGACUCUUCUUCUGGCUGGACCGCCGGAGCUGCCGCCUACUACGUGGGCUACCUGCA |
| | | ACCCCGCACCUUUCUGCUGAAGUACAACGAAAACGGCACCAUCACAGACGCCGUUGACUGCGCUCUGGACCCCCU |
| | | GUCUGAGACUAAGUGCACCCUGAAGUCCUUCACUGUGGAGAAAGGAAUCUACCAGACCUCCAACUUCCGCGUGCA |
| | | GCCUACCGAAUCCAUUGUUCGCUUCCCUAACAUUACUAAUCUGUGCCCCUUCGGAGAAGUUUUCAACGCCACUAG |
| | | AUUCGCUUCCGUGUACGCCUGGAAUCGCAAGCGCAUCUCCAAUUGCGUUGCUGACUACUCUGUGUUAUACAAUUC |
| | | CGCCUCUUUUUCCACUUUCAAGUGCUACGGCGUGUCUCCUACCAAGCUGAACGACCUGUGUUUCACUAACGUGUA |
| | | CGCUGAUUCCUUUGUUAUCCGCGGCGACGAGGUGAGACAGAUCGCCCCUGGCCAGACCGGAAAAAUCGCCGACU |
| | | ACAACUACAAGUUACCCGACGAUUUUACCGGCUGUGUUAUUGCUUGGAACUCUAAUAAUCUGGACUCUAAAGUGG |
| | | GCGGCAAUUACAACUACUUAUAUCGCCUGUUCAGAAAAUCCAACCUGAAACCCUUCGAACGGGACAUUUCCACCGA |
| | | AAUUUAUCAGGCCGGCUCCACCCCCUGCAACGGAGUGGAGGGCUUCAAUUGCUACUUCCCCCUGCAAUCCUACG |
| | | GCUUUCAGCCCACCAACGGCGUGGGAUACCAGCCCUACCGCGUUGUUGUGUUAUCCUUUGAACUGCUGCACGCU |
| | | CCUGCUACUGUGUGCGGACCUAAAAAAUCCACCAAUCUGGUGAAGAACAAGUGUGUGAAUUUUAAUUUUAACGGC |
| | | CUGACCGGCACCGGAGUGCUGACCGAGUCCAACAAAAAGUUUCUGCCCUUUCAGCAGUUCGGCCGGGACAUUGC |
| | | CGACACUACCGACGCUGUGAGGGAUCCUCAGACCCUGGAGAUUCUGGACAUUACCCCUUGCUCCUUCGGAGGAG |
| | | UUUCCGUGAUCACCCCUGGCACCAAUACUUCUAACCAAGUGGCUGUUUUAUACCAGGACGUGAAUUGUACUGAGG |
| | | UGCCUGUGGCCAUCCACGCCGAUCAAUUAACUCCCACCUGGAGAGUGUAUUCCACCGGCUCUAACGUUUUCCAAA |
| | | CCCGCGCUGGCUGCUUAAUCGGCGCUGAGCACGUGAACAACUCCUACGAGUGUGAUAUUCCUAUCGGAGCCGGC |
| | | AUCUGUGCUUCCUACCAGACUCAGACCAACUCCCCCAGACGCGCUAGGUCCGUGGCUUCCCAGUCUAUUAUCGCU |
| | | UAUACUAUGUCCCUGGGAGCCGAGAAUUCUGUUGCCUAUUCCAAUAACUCCAUCGCCAUUCCUACCAACUUUACC |
| | | AUCUCUGUGACCACCGAGAUUCUGCCCGUGUCCAUGACCAAAACUUCUGUGGAUUGUACCAUGUACAUCUGUGGA |
| | | GAUUCCACCGAGUGCUCUAACCUGCUGCUGCAGUACGGCUCCUUUUGCACCCAAUUAAACCGCGCUCUGACUGG |
| | | CAUCGCCGUGGAGCAAGAUAAAAACACCCAAGAGGUGUUCGCUCAGGUGAAGCAGAUCUACAAGACACCUCCCAU |
| | | UAAAGAUUUCGGAGGCUUUAAUUUCUCUCAGAUCCUGCCCGACCCUUCUAAGCCCUCUAAACGCUCCUUCAUCGA |
| | | GGAUCUGCUGUUCAACAAGGUUACCCUGGCCGACGCUGGCUUUAUCAAACAGUACGGCGACUGCUUAGGCGACA |
| | | UCGCCGCCCGCGAUUUAAUUUGCGCUCAGAAGUUCAACGGACUGACCGUGCUGCCUCCUCUGCUGACCGACGAA |
| | | AUGAUCGCCCAAUACACCUCCGCCCUGCUGGCUGGCACUAUCACCUCCGGCUGGACCUUUGGCGCUGGAGCCGC |
| | | UCUGCAAAUCCCUUUCGCUAUGCAGAUGGCUUACCGCUUUAACGGAAUCGGCGUGACCCAGAACGUGUUAUACGA |
| | | AAAUCAGAAAUUAAUUGCCAAUCAAUUUAACUCUGCCAUCGGCAAGAUUCAGGAUUCUCUGUCUUCUACUGCCUCU |
| | | GCCUUAGGAAAACUGCAGGACGUGGUGAAUCAGAACGCCCAGGCCCUGAAUACCCUGGUUAAGCAACUGUCUUCC |
| | | AAUUUCGGCGCUAUCUCCUCUGUGUUAAACGACAUUCUGUCCCGCCUGGAUCCUCCUGAGGCCGAAGUGCAAAUU |
| | | GACCGCUUAAUCACUGGCAGACUGCAAUCCCUGCAAACCUACGUUACUCAACAGCUGAUCAGAGCCGCCGAAAUC |
| | | CGCGCUUCUGCCAACUUAGCUGCUACCAAGAUGUCCGAGUGCGUGCUGGGACAGUCCAAAAGAGUUGACUUUUG |
| | | CGGAAAGGGCUACCACCUGAUGUCCUUUCCCCAGUCCGCCCCCCACGGAGUUGUUUUCCUGCACGUUACUUACG |
| | | UUCCUGCUCAAGAGAAGAACUUCACCACCGCUCCCGCUAUUUGCCACGACGGCAAGGCUCAUUUCCCCAGAGAGG |
| | | GAGUGUUUGUGUCCAACGGCACUCACUGGUUUGUUACUCAGCGCAACUUCUACGAACCUCAGAUCAUCACCACUG |
| | | ACAAUACCUUUGUGUCCGGAAACUGUGACGUGGUUAUCGGCAUUGUGAACAAUACUGUGUACGACCCUUUACAAC |
| | | CUGAGCUGGAUUCUUUUAAAGAAGAACUGGACAAGUACUUCAAGAACCACACCUCCCCCGACGUGGAUCUGGGCG |
| | | ACAUCUCCGGCAUCAACGCCUCUGUGGUGAAUAUUCAGAAGGAGAUUGAUCGCCUGAACGAGGUUGCCAAGAAUC |
| | | UGAACGAGUCUCUGAUCGACCUGCAGGAGCUGGGCAAGUACGAACAGUACAUCAAGUGGCCCUGGUACAUUUGG |
| | | CUGGGCUUUAUUGCUGGACUGAUCGCUAUCGUUAUGGUUACCAUCAUGCUGUGCUGUAUGACCUCUUGCUGCUC |
| | | CUGUCUGAAAGGCUGUUGCUCCUGCGGCUCCUGUUGUAAGUUUGACGAGGACGACUCCGAGCCUGUUCUGAAAG |
| | | GAGUUAAACUGCACUACACCUAAAUGAGACCAUA |
| 143 | 2A0581 | AUAGGUCUCACAUGUUUGUGUUUCUGGUGCUGCUGCCUCUGGUGUCCUCUCAGUGUGUUAACCUGACCACCAGA |
| | | ACCCAACUGCCCCCCGCCUACACUAACUCUUUCACUCGCGGCGUGUACUACCCCGAUAAAGUUUUCAGAUCCUCC |
| | | GUGCUCCAUUCUACCCAAGACCUGUUCUUACCCUUUUUCUCCAACGUGACCUGGUUCCACGCCAUCCACGUGUCC |
| | | GGCACUAACGGAACCAAGCGCUUCGAUAAUCCCGUGCUGCCUUUCAACGACGGAGUUUACUUUGCUUCCACCGAG |
| | | AAGUCCAAUAUUAUCCGGGGCUGGAUCUUCGGCACCACACUGGAUUCCAAAACCCAGUCCUUACUGAUUGUGAAC |
| | | AACGCCACUAACGUGGUGAUCAAGGUGUGUGAGUUCCAGUUCUGUAACGACCCCUUUCUGGGCGUGUAUUAUCA |
| | | UAAAAACAACAAGUCCUGGAUGGAGUCCGAGUUUAGAGUGUAUUCUUCCGCUAACAACUGCACCUUCGAAUACGU |
| | | GUCCCAGCCUUUCCUGAUGGAUCUGGAAGGAAAGCAGGGCAACUUCAAGAACUUACGCGAGUUCGUGUUCAAGAA |
| | | CAUCGACGGCUACUUCAAAAUUUACUCCAAGCACACCCCUAUCAACUUAGUGAGAGAUCUGCCCCAGGGCUUCUC |
| | | CGCUCUGGAACCUCUGGUGGACUUACCCAUUGGCAUCAACAUCACUCGCUUCCAAACACUGCUCGCUCUGCACCG |
| | | CUCUUAUCUGACCCCCGGCGACUCCUCUUCUGGCUGGACCGCCGGCGCCGCUGCCUAUUACGUGGGAUACCUGC |
| | | AACCCCGCACCUUUCUGCUGAAAUACAACGAAAACGGCACCAUCACAGACGCUGUGGACUGCGCUCUGGACCCCC |
| | | UGUCCGAGACUAAGUGCACCCUGAAGUCCUUCACCGUUGAAAAGGGCAUUUAUCAGACUUCCAACUUCCGCGUGC |
| | | AGCCUACUGAGUCCAUUGUGCGCUUCCCUAACAUCACUAACUUGUGUCCUUUCGGCGAAGUGUUCAACGCUACUC |
| | | GCUUCGCCUCCGUGUACGCCUGGAACCGCAAGAGAAUCUCCAAUUGCGUGGCCGACUACUCUGUUCUCUACAACU |
| | | CUGCCUCCUUCUCCACCUUUAAGUGCUACGGCGUGUCCCCCACCAAACUGAACGACCUGUGCUUCACCAACGUGU |
| | | ACGCCGACUCCUUUGUGAUCAGAGGCGACGAGGUGCGCCAGAUCGCCCCCGGCCAGACUGGCAAGAUCGCCGAU |
| | | UACAAUUACAAGCUGCCUGACGACUUUACCGGCUGCGUGAUUGCCUGGAACUCCAACAACCUGGACUCCAAAGUG |
| | | GGCGGCAACUACAACUACCUGUACCGCCUGUUUAGAAAAUCCAACCUGAAGCCCUUCGAGCGGGACAUUUCUACU |
| | | GAGAUCUACCAGGCCGGCUCCACCCCCUGUAACGGCGUGGAAGGCUUCAACUGCUACUUUCCCCUGCAGUCCUA |
| | | CGGCUUCCAGCCUACCAACGGCGUGGGCUACCAGCCCUACCGCGUGGUGGUGUUAUCCUUCGAGCUGCUGCACG |
| | | CCCCUGCCACCGUGUGCGGACCCAAAAAGUCUACUAACCUGGUUAAGAAUAAGUGUGUUAAUUUCAAUUUUAACG |
| | | GCUUAACCGGAACCGGAGUGCUGACCGAGUCCAAUAAAAAGUUCCUGCCCUUUCAGCAGUUUGGCCGCGACAUCG |
| | | CCGAUACUACCGACGCCGUGCGCGAUCCCCAGACCCUGGAGAUCCUGGACAUUACCCCCUGCUCUUUCGGCGGA |
| | | GUGUCCGUGAUCACCCCCGGCACCAAUACCUCCAAUCAGGUGGCCGUUCUGUACCAGGACGUGAAUUGCACCGAA |
| | | GUGCCCGUGGCCAUUCACGCCGACCAGUUAACUCCUACUUGGAGAGUGUACUCCACCGGCUCUAACGUGUUUCA |
| | | GACCCGCGCCGGCUGUCUGAUCGGCGCCGAGCACGUGAACAACUCCUACGAGUGCGACAUUCCCAUUGGCGCCG |
| | | GCAUCUGCGCCUCCUACCAAACCCAGACUAACUCUCCCAGACGCGCCCGCUCUGUGGCCUCUCAGUCCAUCAUCG |
| | | CUUACACUAUGUCCCUGGGCGCCGAGAACUCCGUGGCCUAUUCCAACAACUCCAUCGCCAUCCCCACUAACUUCA |
| | | CCAUCUCUGUGACCACCGAAAUCCUCCCCGUGUCUAUGACUAAGACUUCCGUGGACUGUACCAUGUACAUUUGCG |
| | | GCGACUCCACCGAGUGUUCCAAUCUGCUGCUGCAGUACGGCUCCUUUUGCACCCAGCUGAACCGCGCCCUGACC |
| | | GGCAUCGCUGUUGAGCAGGACAAGAAUACCCAGGAGGUGUUUGCUCAGGUGAAGCAAAUCUAUAAGACCCCUCCC |
| | | AUCAAGGACUUUGGCGGCUUCAACUUUUCCCAGAUCCUGCCCGACCCUUCCAAGCCCUCCAAACGCUCUUUCAUC |
| | | GAAGAUCUGCUGUUCAAUAAAGUGACCCUGGCCGACGCCGGCUUCAUCAAGCAGUACGGAGACUGUCUGGGAGA |
| | | CAUUGCUGCUCGCGACCUGAUUUGCGCCCAGAAGUUCAACGGCCUGACCGUGCUGCCCCCUCUGCUGACCGACG |
| | | AGAUGAUCGCUCAGUACACCUCCGCCCUGCUGGCUGGCACUAUCACCUCCGGCUGGACUUUCGGCGCUGGCGCC |
| | | GCCCUGCAGAUUCCCUUCGCCAUGCAGAUGGCUUAUCGCUUCAACGGAAUCGGAGUGACACAGAACGUGCUGUA |
| | | CGAGAAUCAGAAACUGAUCGCUAACCAGUUUAAUUCCGCCAUUGGCAAGAUCCAGGACUCCCUGUCCUCUACUGC |
| | | UUCCGCCCUGGGCAAGCUGCAGGACGUGGUGAACCAGAACGCCCAGGCUCUGAACACCCUGGUGAAGCAACUGU |
| | | CUUCUAACUUUGGAGCCAUUUCUUCCGUGCUGAACGACAUCCUGUCCAGACUGGAUCCUCCUGAGGCCGAGGUG |
| | | CAGAUCGACCGCCUGAUCACUGGCCGCCUGCAGUCCCUGCAGACUUACGUUACCCAGCAAUUAAUCCGCGCUGCC |
| | | GAGAUCCGCGCCUCCGCCAACCUGGCCGCCACCAAGAUGUCCGAGUGCGUUCUGGGCCAGUCCAAACGCGUUGA |
| | | CUUCUGCGGCAAGGGCUACCACCUGAUGUCCUUCCCCCAGUCCGCUCCCCACGGAGUGGUGUUCCUGCACGUGA |
| | | CCUACGUGCCUGCCCAGGAGAAGAACUUCACCACCGCCCCCGCCAUUUGCCACGACGGAAAGGCCCACUUCCCCC |
| | | GCGAGGGAGUGUUCGUUUCUAACGGCACUCACUGGUUCGUGACCCAGAGAAACUUCUACGAGCCCCAGAUUAUUA |
| | | CCACCGACAAUACCUUCGUUUCUGGCAACUGCGACGUGGUUAUCGGCAUCGUGAACAACACCGUGUACGACCCCC |
| | | UGCAACCCGAGCUGGAUUCUUUCAAGGAGGAGCUGGAUAAGUACUUUAAAAACCAUACCUCCCCCGACGUGGACC |
| | | UGGGCGACAUCUCUGGCAUCAACGCCUCCGUGGUGAAUAUCCAGAAGGAGAUCGAUCGCCUGAACGAGGUGGCC |
| | | AAGAACUUAAACGAAUCUCUGAUCGACCUGCAGGAGCUGGGCAAGUACGAGCAGUAUAUUAAGUGGCCCUGGUAU |
| | | AUCUGGUUAGGCUUUAUCGCCGGCCUGAUCGCCAUUGUUAUGGUGACCAUCAUGCUGUGCUGCAUGACCUCCUG |
| | | CUGUUCCUGUUUAAAAGGCUGCUGCUCCUGCGGAUCCUGCUGUAAAUUCGACGAAGACGACUCCGAGCCCGUGC |
| | | UGAAAGGAGUUAAGUUACACUAUACCUAAAUGAGACCAUA |
| 144 | 2A1581 | AUAGGUCUCACAUGUUCGUGUUCCUGGUGCUGCUGCCCCUGGUGUCCUCCCAGUGCGUCAACCUGACCACCCGG |
| | | ACCCAACUGCCCCCUGCCUAUACCAAUUCCUUCACCCGGGGCGUCUACUACCCCGACAAGGUGUUCCGGUCCUCU |
| | | GUGCUCCACUCCACCCAGGACCUCUUCCUCCCCUUUUUCUCCAACGUGACCUGGUUCCACGCCAUCCACGUGUCU |
| | | GGCACAAACGGAACCAAGCGCUUCGACAACCCUGUGCUCCCCUUCAACGACGGCGUGUACUUCGCCUCCACCGAG |
| | | AAGUCCAACAUCAUCCGCGGCUGGAUCUUCGGCACCACCCUCGACUCUAAGACCCAGUCCCUGCUGAUUGUGAAU |
| | | AACGCCACCAACGUGGUGAUCAAGGUGUGCGAAUUUCAGUUCUGCAACGACCCCUUCCUGGGCGUUUACUACCAU |
| | | AAGAACAACAAGUCCUGGAUGGAGUCCGAGUUCCGGGUGUAUUCCUCUGCCAAUAACUGCACCUUUGAGUACGUG |
| | | UCCCAGCCCUUCCUGAUGGACCUGGAGGGCAAGCAGGGCAAUUUUAAAAACCUGCGCGAGUUCGUGUUCAAGAAC |
| | | AUCGACGGCUACUUCAAGAUCUAUUCCAAGCACACCCCUAUCAACCUGGUGCGCGACCUGCCCCAGGGCUUUUCC |
| | | GCUCUGGAGCCCCUGGUUGACCUGCCCAUCGGCAUCAACAUCACCCGCUUCCAGACUUUACUGGCCCUGCACCG |
| | | CUCCUACCUGACCCCCGGCGAUUCCUCUUCUGGCUGGACAGCCGGAGCCGCUGCCUACUACGUGGGAUACCUGC |
| | | AGCCCCGCACCUUCCUGCUGAAGUACAACGAGAACGGAACCAUUACAGACGCUGUCGACUGUGCUCUGGAUCCCC |
| | | UGUCCGAAACAAAGUGCACCCUGAAGUCCUUCACCGUGGAGAAGGGCAUCUACCAGACCUCCAACUUCCGCGUGC |
| | | AGCCCACCGAGUCCAUCGUGAGAUUCCCCAACAUCACUAACCUGUGCCCCUUCGGCGAGGUGUUCAACGCCACCC |
| | | GCUUCGCCUCCGUGUACGCCUGGAACCGCAAGCGCAUCUCCAACUGCGUUGCCGACUACUCUGUGCUCUACAAC |
| | | UCCGCCUCCUUCUCCACCUUCAAGUGCUACGGCGUGUCCCCCACCAAGCUGAACGACCUGUGCUUCACCAACGUG |
| | | UACGCCGACUCCUUCGUGAUCCGCGGCGACGAGGUGCGCCAGAUCGCCCCCGGCCAGACCGGAAAGAUCGCCGA |
| | | UUACAAUUACAAGCUGCCCGACGACUUCACCGGCUGCGUGAUCGCCUGGAACUCCAACAAUCUGGACUCCAAGGU |
| | | UGGCGGCAACUACAACUACCUGUACCGCCUGUUCCGCAAGUCCAACCUGAAGCCCUUCGAGCGGGACAUUUCCAC |
| | | AGAGAUUUACCAGGCUGGCUCCACCCCCUGUAACGGAGUGGAGGGCUUCAACUGCUACUUCCCCCUGCAGUCCU |
| | | ACGGCUUCCAGCCCACCAACGGCGUGGGCUACCAGCCCUACCGCGUGGUGGUGCUGUCCUUCGAGCUGCUGCAC |
| | | GCCCCUGCCACCGUGUGCGGCCCCAAGAAGUCCACCAACCUGGUGAAGAACAAGUGCGUGAACUUCAACUUCAAC |
| | | GGACUGACAGGCACCGGCGUGCUGACCGAGUCCAACAAGAAGUUCCUGCCCUUCCAGCAGUUCGGCCGGGACAU |
| | | UGCCGACACCACCGACGCCGUGCGCGACCCCCAGACCCUGGAGAUCCUGGACAUCACCCCCUGCUCCUUCGGCG |
| | | GAGUGUCCGUGAUCACCCCCGGCACCAACACCUCCAACCAGGUUGCCGUGCUGUACCAGGACGUUAACUGCACC |
| | | GAGGUGCCCGUGGCCAUCCACGCCGACCAGCUGACCCCCACCUGGCGCGUGUACUCCACCGGCUCCAACGUGUU |
| | | CCAGACCCGCGCCGGCUGCCUGAUCGGCGCCGAGCACGUGAACAACUCCUACGAGUGCGACAUCCCCAUUGGCG |
| | | CCGGCAUCUGCGCCUCCUACCAGACCCAGACCAACUCCCCUCGCCGCGCCCGGUCCGUCGCCUCCCAGUCCAUC |
| | | AUCGCCUACACCAUGUCCCUGGGCGCCGAGAACUCCGUGGCCUACUCCAACAACUCCAUUGCCAUCCCCACCAAC |
| | | UUCACCAUCUCCGUGACCACCGAAAUCCUCCCCGUGUCCAUGACCAAGACCUCCGUGGACUGCACCAUGUACAUC |
| | | UGCGGCGACUCCACCGAGUGCUCCAACCUGCUGCUGCAGUACGGCUCCUUCUGCACCCAGCUGAAUCGCGCCCU |
| | | GACCGGCAUCGCCGUGGAGCAGGACAAGAACACCCAAGAGGUGUUCGCCCAGGUGAAGCAGAUCUACAAGACACC |
| | | UCCCAUCAAGGACUUCGGCGGCUUCAACUUCUCCCAGAUCCUGCCCGACCCCUCCAAGCCCUCCAAGCGCUCCUU |
| | | CAUCGAGGACCUGCUGUUCAACAAGGUGACCCUGGCCGACGCCGGCUUCAUCAAGCAGUACGGCGACUGUCUGG |
| | | GAGACAUUGCCGCCCGCGACCUGAUCUGCGCCCAGAAGUUCAACGGCUUAACCGUGCUGCCCCCUCUGCUGACC |
| | | GACGAGAUGAUCGCCCAGUACACAUCCGCUCUGCUGGCCGGCACCAUCACCUCCGGCUGGACCUUUGGCGCUGG |
| | | CGCUGCCCUGCAGAUCCCCUUCGCUAUGCAGAUGGCCUACCGCUUCAACGGCAUCGGCGUGACCCAGAACGUGC |
| | | UGUACGAGAACCAGAAGCUGAUCGCCAACCAGUUCAACUCCGCCAUCGGCAAGAUCCAGGACUCCCUGUCCUCCA |
| | | CCGCCUCCGCCCUGGGCAAGCUGCAGGACGUGGUGAACCAGAACGCCCAGGCCCUGAACACCCUGGUGAAGCAG |
| | | CUGUCCUCUAACUUCGGCGCCAUCUCCUCCGUGCUGAACGACAUUCUGUCCCGCCUGGAUCCUCCUGAGGCCGA |
| | | GGUGCAAAUCGAUCGCCUCAUCACCGGCCGCCUGCAGUCCCUGCAGACCUACGUGACACAGCAGCUGAUCCGCG |
| | | CCGCCGAGAUCCGCGCCUCCGCCAACCUGGCCGCCACCAAGAUGUCUGAGUGCGUGCUGGGCCAGUCCAAACGC |
| | | GUUGACUUCUGCGGCAAGGGCUACCACCUGAUGUCCUUCCCCCAGUCCGCCCCCCACGGCGUGGUGUUCCUGCA |
| | | CGUGACCUACGUGCCCGCCCAGGAGAAGAACUUCACUACCGCCCCCGCCAUCUGCCACGACGGCAAGGCCCACUU |
| | | CCCCCGCGAGGGCGUGUUCGUGUCCAACGGCACCCACUGGUUCGUGACCCAGCGCAACUUCUACGAGCCCCAGA |
| | | UCAUCACCACCGACAACACCUUCGUGUCCGGCAACUGCGACGUGGUGAUUGGCAUCGUGAACAACACCGUGUACG |
| | | ACCCCCUGCAGCCCGAGCUGGACUCCUUCAAGGAGGAGCUGGACAAGUACUUUAAGAACCACACCUCCCCCGACG |
| | | UGGACCUGGGCGACAUCUCCGGCAUCAACGCCUCCGUGGUGAACAUCCAGAAGGAGAUCGACCGCCUGAACGAG |
| | | GUGGCCAAGAACCUGAACGAGUCCCUGAUCGACCUGCAGGAGCUGGGCAAGUACGAACAGUACAUCAAGUGGCCC |
| | | UGGUACAUCUGGCUGGGCUUCAUCGCCGGCCUGAUCGCCAUCGUGAUGGUGACCAUCAUGCUGUGCUGCAUGAC |
| | | CUCUUGCUGUUCCUGUCUGAAGGGCUGCUGCUCCUGCGGCUCCUGCUGCAAGUUCGACGAGGACGACUCCGAGC |
| | | CCGUGCUGAAGGGCGUGAAGCUGCACUACACCUAAAUGAGACCAUA |
| 145 | 2A2581 | AUAGGUCUCACAUGUUCGUGUUCCUGGUGCUGCUCCCCCUGGUGAGCUCCCAGUGCGUCAACCUGACCACACGG |
| | | ACCCAGCUGCCCCCCGCCUAUACCAACAGCUUCACCCGCGGCGUCUACUACCCCGACAAGGUGUUCCGGAGCAGC |
| | | GUGCUGCACAGCACCCAGGACCUCUUCCUCCCCUUUUUUAGCAACGUGACCUGGUUCCACGCCAUCCACGUGAG |
| | | UGGCACCAACGGCACCAAGCGGUUCGACAACCCUGUGCUCCCCUUCAACGACGGCGUGUACUUCGCUAGCACAGA |
| | | AAAAAGUAACAUCAUUCGGGGCUGGAUCUUCGGCACCACACUGGAUAGCAAAACCCAGAGCCUGCUGAUUGUGAA |
| | | UAACGCCACCAACGUGGUGAUCAAGGUGUGCGAGUUCCAGUUCUGCAACGACCCCUUCCUGGGCGUGUACUACC |
| | | ACAAGAACAACAAGAGCUGGAUGGAGAGCGAGUUCCGCGUGUAUAGCUCUGCCAACAACUGCACCUUCGAGUACG |
| | | UGAGCCAGCCCUUCCUGAUGGACCUGGAGGGCAAGCAGGGCAAUUUCAAAAACCUGCGGGAGUUCGUGUUCAAG |
| | | AACAUCGACGGCUACUUCAAGAUCUACAGCAAGCACACCCCCAUCAACCUGGUGCGGGACCUGCCCCAGGGCUUU |
| | | AGCGCUCUGGAGCCCCUGGUGGACCUGCCCAUCGGCAUCAACAUCACCCGCUUCCAAACCCUGUUGGCCCUGCA |
| | | CCGGAGCUACCUGACCCCUGGCGAUAGCAGUAGUGGCUGGACAGCCGGAGCCGCCGCCUACUACGUGGGAUACC |
| | | UGCAGCCCCGGACCUUCCUGCUGAAGUACAACGAGAACGGCACAAUCACAGACGCCGUGGACUGCGCCCUGGAC |
| | | CCCCUGAGCGAAACAAAGUGCACCCUCAAAAGUUUCACCGUGGAGAAGGGCAUCUACCAGACCAGCAACUUCCGG |
| | | GUGCAGCCCACCGAGAGCAUCGUGCGGUUCCCCAACAUCACCAACCUGUGCCCCUUCGGCGAGGUGUUCAACGC |
| | | CACCCGGUUCGCCAGCGUGUACGCCUGGAACCGGAAGCGGAUCAGCAACUGCGUGGCCGACUACAGCGUGCUGU |
| | | AUAACAGUGCCAGCUUCAGCACCUUCAAGUGCUACGGCGUGAGCCCCACCAAGCUGAACGACCUGUGCUUCACCA |
| | | ACGUGUACGCCGACAGCUUCGUGAUCCGGGGCGACGAGGUGCGGCAGAUCGCCCCCGGCCAGACCGGCAAGAUC |
| | | GCCGAUUACAAUUACAAGCUGCCCGACGAUUUCACCGGCUGCGUGAUCGCCUGGAACAGCAACAACCUGGACAGC |
| | | AAGGUGGGCGGCAACUACAACUACCUGUACCGGCUGUUCCGCAAAAGUAACCUGAAGCCCUUCGAGCGGGACAUC |
| | | AGCACCGAGAUCUACCAGGCCGGCAGUACCCCUUGCAACGGCGUCGAGGGCUUCAACUGCUACUUCCCCCUGCA |
| | | GAGCUACGGCUUCCAGCCCACCAACGGCGUGGGCUACCAGCCCUACCGGGUGGUGGUUCUGAGCUUCGAGCUGC |
| | | UGCACGCCCCUGCCACCGUGUGCGGCCCCAAGAAAAGCACCAACCUGGUGAAGAACAAGUGCGUGAAUUUCAAUU |
| | | UUAACGGACUCACCGGAACCGGCGUGCUGACCGAGAGCAACAAGAAGUUCCUGCCCUUCCAGCAAUUCGGCCGG |
| | | GAUAUUGCCGACACCACUGACGCCGUGCGGGACCCCCAGACCCUGGAGAUCCUGGACAUCACACCCUGUAGCUU |
| | | CGGCGGGGUGAGCGUGAUCACCCCCGGCACCAACACCAGCAACCAGGUGGCCGUGCUGUACCAGGACGUGAACU |
| | | GCACCGAGGUGCCCGUGGCCAUCCACGCCGACCAGCUGACCCCCACCUGGGGGGUGUACAGCACCGGCAGCAAC |
| | | GUGUUCCAGACCCGGGCCGGCUGCCUGAUCGGCGCCGAGCACGUGAAUAACAGCUACGAGUGCGACAUCCCCAU |
| | | CGGAGCCGGAAUCUGCGCCAGCUACCAGACCCAGACCAACAGCCCCCGGAGAGCCCGGAGCGUGGCCAGCCAGA |
| | | GCAUUAUUGCCUACACCAUGAGCCUGGGCGCCGAGAACAGCGUGGCCUACAGCAACAAUAGUAUUGCCAUCCCCA |
| | | CCAACUUCACCAUCAGCGUGACCACCGAAAUCCUCCCCGUGAGCAUGACCAAGACCAGCGUGGACUGCACCAUGU |
| | | ACAUCUGCGGCGACAGCACCGAGUGCAGCAACCUGCUCCUGCAGUACGGCAGCUUCUGCACCCAGCUGAACCGG |
| | | GCCCUGACCGGAAUUGCCGUGGAGCAGGACAAGAACACCCAGGAGGUGUUCGCCCAGGUGAAGCAGAUCUACAA |
| | | GACACCUCCCAUCAAGGACUUCGGCGGCUUCAACUUCAGCCAGAUCCUGCCCGACCCCAGCAAGCCCAGCAAGCG |
| | | GAGCUUCAUCGAGGACCUGUUGUUCAACAAGGUGACCCUGGCCGACGCCGGCUUCAUCAAGCAGUACGGCGACU |
| | | GUCUGGGAGAUAUUGCCGCACGGGACCUGAUCUGCGCCCAGAAGUUCAACGGCCUGACCGUGCUGCCCCCACUG |
| | | CUGACCGACGAGAUGAUCGCCCAGUACACAAGCGCUCUGCUCGCCGGCACCAUCACCAGCGGCUGGACCUUCGG |
| | | CGCCGGCGCUGCCCUGCAGAUCCCCUUCGCCAUGCAGAUGGCCUACCGGUUCAACGGCAUCGGCGUGACCCAGA |
| | | ACGUGCUGUACGAGAACCAGAAGCUGAUCGCCAACCAGUUCAACAGCGCCAUCGGCAAGAUCCAGGACAGCCUGA |
| | | GCUCCACCGCCAGCGCCCUGGGCAAGCUGCAGGACGUGGUGAACCAGAACGCCCAGGCCCUGAACACCCUGGUG |
| | | AAGCAGCUGAGCAGCAACUUCGGCGCCAUCAGCUCUGUGCUGAACGACAUCCUGAGCCGGCUGGAUCCUCCUGA |
| | | GGCCGAGGUGCAAAUCGAUCGGCUCAUCACCGGCCGGCUGCAGAGCCUGCAGACCUACGUGACACAGCAGCUGA |
| | | UCCGGGCCGCCGAGAUCCGGGCCAGCGCCAACCUGGCCGCUACCAAGAUGAGCGAGUGCGUGCUGGGCCAGAGC |
| | | AAGCGGGUGGACUUCUGCGGCAAGGGCUACCACCUGAUGAGCUUCCCCCAGAGCGCCCCCCACGGCGUGGUGUU |
| | | CCUGCACGUGACCUACGUGCCCGCCCAGGAGAAGAACUUCACCACCGCCCCCGCCAUCUGCCACGACGGCAAGG |
| | | CCCACUUCCCCCGGGAGGGCGUGUUCGUGAGCAACGGCACCCACUGGUUCGUGACCCAGCGGAACUUCUACGAG |
| | | CCCCAGAUCAUAACCACAGACAACACCUUCGUGAGCGGCAACUGCGACGUGGUCAUCGGCAUCGUGAACAACACC |
| | | GUGUACGACCCCCUGCAGCCCGAGCUGGACAGCUUCAAGGAGGAACUGGACAAGUACUUCAAGAACCACACCAGC |
| | | CCCGACGUGGACCUGGGCGACAUCAGCGGCAUCAACGCCAGCGUGGUGAACAUCCAGAAGGAGAUCGACCGGCU |
| | | GAACGAGGUGGCCAAGAACCUGAACGAGAGCCUGAUCGACCUGCAGGAGCUGGGCAAGUACGAGCAGUACAUCAA |
| | | GUGGCCCUGGUACAUCUGGCUGGGCUUUAUUGCCGGCCUGAUCGCCAUCGUGAUGGUGACCAUCAUGCUGUGCU |
| | | GUAUGACAAGCUGUUGCAGUUGCCUGAAGGGCUGCUGCAGCUGCGGCAGCUGCUGCAAGUUCGACGAGGACGAC |
| | | AGCGAGCCCGUGCUGAAGGGCGUGAAGCUGCACUACACCUAAAUGAGACCAUA |
| 146 | 2A3581 | AUAGGUCUCACACAUUAAAACAGCCUGUGGGUUGAUCCCACCCACAGGCCCAUUGGGCGCUAGCACUCUGGUAUC |
| | | ACGGUACCUUUGUGCGCCUGUUUUAUACCCCCUCCCCCAACUGUAACUUAGAAGUAACACACACCGAUCAACAGU |
| | | CAGCGUGGCACACCAGCCACGUUUUGAUCAAGCACUUCUGUUACCCCGGACUGAGUAUCAAUAGACUGCUCACGC |
| | | GGUUGAAGGAGAAAGCGUUCGUUAUCCGGCCAACUACUUCGAAAAACCUAGUAACACCGUGGAAGUUGCAGAGUG |
| | | UUUCGCUCAGCACUACCCCAGUGUAGAUCAGGUCGAUGAGUCACCGCAUUCCCCACGGGCGACCGUGGCGGUGG |
| | | CUGCGUUGGCGGCCUGCCCAUGGGGAAACCCAUGGGACGCUCUAAUACAGACAUGGUGCGAAGAGUCUAUUGAG |
| | | CUAGUUGGUAGUCCUCCGGCCCCUGAAUGCGGCUAAUCCUAACUGCGGAGCACACACCCUCAAGCCAGAGGGCA |
| | | GUGUGUCGUAACGGGCAACUCUGCAGCGGAACCGACUACUUUGGGUGUCCGUGUUUCAUUUUAUUCCUAUACUG |
| | | GCUGCUUAUGGUGACAAUUGAGAGAUCGUUACCAUAUAGCUAUUGGAUUGGCCAUCCGGUGACUAAUAGAGCUAU |
| | | UAUAUAUCCCUUUGUUGGGUUUAUACCACUUAGCUUGAAAGAGGUUAAAACAUUACAAUUCAUUGUUAAGUUGAAU |
| | | ACAGCAACAUGUUUGUUUUCUUAGUUUUAUUACCUUUAGUUUCUUCCCAGUGUGUUAAUUUAACUACUAGAACUC |
| | | AAUUACCUCCUGCUUAUACUAAUUCUUUUACUAGAGGAGUGUAUUAUCCUGAUAAGGUUUUUAGAUCUUCUGUUC |
| | | UGCACUCUACUCAGGAUUUGUUCUUGCCCUUCUUCUCUAACGUGACCUGGUUCCACGCUAUUCACGUUUCUGGAA |
| | | CCAACGGCACUAAGAGAUUUGAUAAUCCCGUUUUGCCUUUUAACGACGGAGUUUAUUUCGCCUCUACCGAAAAAU |
| | | CUAACAUCAUUAGGGGCUGGAUCUUUGGAACUACCCUGGAUUCUAAAACUCAAUCUUUAUUGAUUGUGAAUAACG |
| | | CUACCAACGUUGUGAUUAAAGUUUGUGAAUUUCAAUUUUGUAACGAUCCUUUUCUGGGAGUUUAUUAUCAUAAAA |
| | | ACAAUAAAUCUUGGAUGGAAUCUGAAUUCCGCGUUUAUUCUUCUGCCAAUAACUGUACCUUUGAGUACGUUUCUC |
| | | AACCUUUCUUAAUGGAUUUAGAAGGAAAACAAGGCAAUUUCAAAAACUUAAGAGAAUUUGUUUUUAAAAAUAUUGA |
| | | CGGAUAUUUUAAAAUUUAUUCUAAGCAUACUCCUAUUAAUUUAGUGAGAGACUUACCCCAAGGAUUUUCUGCUUUA |
| | | GAACCUUUAGUUGACUUACCUAUUGGAAUCAAUAUCACUAGAUUUCAAACUUUAUUAGCUUUACAUAGAUCUUAUU |
| | | UAACUCCUGGAGAUUCUUCCUCCGGCUGGACAGCCGGAGCCGCUGCUUAUUACGUUGGAUAUUUACAGCCUAGA |
| | | ACUUUUCUGCUGAAAUAUAACGAAAACGGAACCAUUACCGACGCCGUUGACUGUGCUCUGGAUCCUUUAUCUGAA |
| | | ACCAAGUGUACUUUAAAAUCUUUUACCGUUGAAAAAGGAAUUUAUCAAACUUCUAAUUUUAGAGUUCAACCUACUG |
| | | AAUCUAUCGUUAGAUUCCCUAAUAUUACUAAUUUGUGUCCCUUUGGAGAGGUUUUCAACGCUACUAGAUUUGCCU |
| | | CUGUUUACGCUUGGAAUCGCAAGAGAAUUUCUAAUUGUGUGGCCGAUUACUCCGUUUUGUAUAACUCUGCUUCUU |
| | | UCUCUACUUUUAAGUGUUACGGAGUUUCCCCUACUAAACUGAACGAUUUGUGUUUUACUAACGUUUACGCUGAUU |
| | | CUUUUGUUAUUAGAGGCGACGAAGUUAGACAAAUUGCUCCUGGACAAACUGGAAAGAUUGCUGACUAUAACUAUA |
| | | AACUGCCUGACGAUUUUACCGGCUGUGUUAUUGCUUGGAAUUCUAAUAAUUUAGAUUCUAAAGUUGGAGGAAAUU |
| | | AUAAUUAUUUAUAUAGAUUAUUUAGAAAAUCUAAUUUAAAACCUUUUGAAAGAGAUAUUUCCACUGAGAUUUACCA |
| | | GGCUGGAUCUACUCCUUGUAACGGCGUUGAGGGAUUUAAUUGUUAUUUUCCUUUACAAUCUUACGGAUUUCAACC |
| | | UACCAACGGAGUUGGAUAUCAACCUUACAGAGUUGUGGUGCUGUCUUUUGAAUUAUUACACGCUCCCGCUACUGU |
| | | UUGCGGCCCUAAAAAGUCUACUAAUCUGGUUAAGAAUAAGUGUGUGAAUUUUAAUUUCAACGGAUUGACCGGAAC |
| | | UGGAGUUCUGACUGAAUCUAAUAAAAAAUUUUUACCCUUUCAACAAUUUGGAAGGGAUAUCGCUGAUACUACUGAC |
| | | GCUGUUAGAGAUCCUCAAACUUUAGAAAUCCUGGAUAUUACUCCUUGUUCUUUUGGAGGAGUUUCCGUUAUUACU |
| | | CCUGGCACUAAUACUUCUAAUCAAGUUGCUGUUUUAUAUCAAGACGUUAAUUGCACCGAAGUUCCUGUUGCUAUU |
| | | CACGCUGAUCAGUUAACUCCUACUUGGAGAGUUUAUUCUACUGGAUCUAACGUUUUUCAAACCAGAGCUGGCUGU |
| | | UUAAUCGGAGCUGAACACGUGAAUAAUUCUUACGAGUGCGAUAUUCCCAUCGGAGCUGGAAUUUGUGCCUCUUAU |
| | | CAAACUCAAACCAAUUCUCCUAGGAGGGCUAGAUCCGUUGCCUCUCAAUCUAUUAUUGCUUAUACCAUGUCUUUA |
| | | GGAGCUGAAAAUUCCGUUGCUUACUCUAACAAUUCUAUUGCUAUUCCUACUAACUUUACUAUUUCUGUUACUACU |
| | | GAAAUUCUGCCCGUGUCCAUGACUAAAACUUCUGUUGAUUGUACUAUGUAUAUUUGUGGAGAUUCCACUGAGUGU |
| | | UCUAACCUGUUAUUACAAUACGGAUCUUUUUGUACUCAAUUAAAUAGAGCUCUGACUGGAAUUGCUGUUGAACAG |
| | | GACAAAAAUACUCAAGAAGUUUUUGCCCAAGUUAAACAGAUUUAUAAAACUCCUCCUAUUAAAGAUUUUGGAGGAU |
| | | UUAAUUUCUCUCAAAUUUUACCUGACCCUUCUAAGCCUUCCAAAAGGUCUUUCAUCGAAGAUUUAUUAUUUAAUAA |
| | | AGUUACCUUAGCCGACGCUGGAUUUAUUAAGCAAUACGGAGAUUGCUUAGGCGAUAUCGCUGCUAGAGACUUAAU |
| | | UUGUGCUCAAAAAUUUAACGGAUUAACUGUUUUACCUCCCCUGUUAACUGACGAAAUGAUCGCUCAAUAUACUUCU |
| | | GCUCUGUUAGCUGGAACUAUUACUUCUGGCUGGACUUUUGGAGCUGGAGCUGCUUUACAAAUUCCCUUUGCUAU |
| | | GCAGAUGGCUUAUAGAUUUAACGGAAUUGGAGUUACUCAAAACGUUUUAUACGAAAAUCAAAAAUUAAUUGCUAAU |
| | | CAAUUUAAUUCUGCUAUUGGAAAAAUUCAAGAUUCCCUGUCUUCUACUGCUUCCGCUCUGGGAAAAUUACAAGAC |
| | | GUUGUUAAUCAAAACGCUCAAGCUUUGAAUACCUUAGUUAAACAAUUAUCUUCCAAUUUUGGAGCUAUUUCUUCUG |
| | | UUUUAAACGACAUUUUAUCUAGAUUAGAUCCCCCUGAAGCUGAAGUUCAGAUUGACAGAUUGAUUACCGGAAGAU |
| | | UACAAUCUUUACAAACUUACGUUACUCAACAACUGAUUAGAGCUGCUGAAAUUAGAGCCUCUGCUAAUUUAGCUGC |
| | | UACUAAGAUGUCUGAGUGUGUUUUAGGACAGUCCAAAAGAGUUGAUUUUUGUGGAAAAGGCUAUCAUCUGAUGUC |
| | | UUUUCCUCAAUCUGCUCCUCACGGAGUUGUGUUUUUACACGUUACUUACGUUCCUGCUCAAGAGAAGAAUUUUAC |
| | | UACUGCUCCUGCUAUUUGUCACGACGGAAAAGCUCAUUUUCCUAGAGAAGGAGUUUUUGUUUCUAACGGAACUCA |
| | | UUGGUUUGUUACUCAAAGAAACUUUUACGAACCUCAGAUUAUUACCACUGAUAAUACCUUUGUUUCUGGCAAUUG |
| | | UGACGUGGUUAUUGGAAUUGUUAAUAAUACUGUUUACGAUCCUUUACAACCCGAACUGGAUUCUUUUAAAGAAGA |
| | | AUUAGAUAAAUACUUUAAAAAUCAUACUUCUCCCGACGUUGAUUUAGGAGAUAUCUCUGGAAUUAACGCUUCUGUU |
| | | GUGAAUAUUCAAAAGGAAAUUGAUAGAUUAAACGAAGUGGCUAAAAAUUUAAACGAGUCUUUAAUUGAUUUACAGG |
| | | AACUGGGAAAAUACGAACAAUAUAUUAAGUGGCCUUGGUACAUUUGGUUAGGCUUUAUUGCUGGACUGAUUGCUA |
| | | UUGUGAUGGUUACUAUCAUGUUGUGUUGCAUGACUUCUUGUUGUUCCUGUUUAAAAGGCUGUUGCUCUUGUGGA |
| | | UCUUGUUGCAAAUUUGACGAAGACGAUUCUGAACCUGUUCUGAAAGGAGUUAAACUGCAUUAUACUUAAAUGAGA |
| | | CCAUA |
| 147 | 2A4581 | AUAGGUCUCACACAUUAAAACAGCCUGUGGGUUGAUCCCACCCACAGGCCCAUUGGGCGCUAGCACUCUGGUAUC |
| | | ACGGUACCUUUGUGCGCCUGUUUUAUACCCCCUCCCCCAACUGUAACUUAGAAGUAACACACACCGAUCAACAGU |
| | | CAGCGUGGCACACCAGCCACGUUUUGAUCAAGCACUUCUGUUACCCCGGACUGAGUAUCAAUAGACUGCUCACGC |
| | | GGUUGAAGGAGAAAGCGUUCGUUAUCCGGCCAACUACUUCGAAAAACCUAGUAACACCGUGGAAGUUGCAGAGUG |
| | | UUUCGCUCAGCACUACCCCAGUGUAGAUCAGGUCGAUGAGUCACCGCAUUCCCCACGGGCGACCGUGGCGGUGG |
| | | CUGCGUUGGCGGCCUGCCCAUGGGGAAACCCAUGGGACGCUCUAAUACAGACAUGGUGCGAAGAGUCUAUUGAG |
| | | CUAGUUGGUAGUCCUCCGGCCCCUGAAUGCGGCUAAUCCUAACUGCGGAGCACACACCCUCAAGCCAGAGGGCA |
| | | GUGUGUCGUAACGGGCAACUCUGCAGCGGAACCGACUACUUUGGGUGUCCGUGUUUCAUUUUAUUCCUAUACUG |
| | | GCUGCUUAUGGUGACAAUUGAGAGAUCGUUACCAUAUAGCUAUUGGAUUGGCCAUCCGGUGACUAAUAGAGCUAU |
| | | UAUAUAUCCCUUUGUUGGGUUUAUACCACUUAGCUUGAAAGAGGUUAAAACAUUACAAUUCAUUGUUAAGUUGAAU |
| | | ACAGCAACAUGUUCGUGUUCUUAGUGCUGUUACCCCUGGUUUCUUCUCAGUGUGUCAAUUUAACCACCAGAACCC |
| | | AACUGCCUCCUGCCUAUACUAAUUCCUUUACCCGCGGAGUUUACUAUCCCGAUAAGGUUUUUCGCUCCUCUGUUU |
| | | UACACUCUACUCAGGACUUAUUCUUACCCUUCUUCUCCAACGUUACUUGGUUUCACGCCAUUCACGUUUCUGGAA |
| | | CCAACGGAACUAAGAGAUUCGAUAAUCCUGUGUUACCUUUCAACGACGGAGUUUAUUUUGCUUCCACUGAAAAAU |
| | | CCAAUAUCAUUAGGGGCUGGAUCUUUGGAACCACUUUAGAUUCCAAGACUCAAUCUUUAUUAAUUGUGAAUAACG |
| | | CCACCAACGUGGUUAUCAAAGUGUGUGAAUUCCAAUUUUGUAACGACCCUUUCUUAGGCGUGUAUUAUCACAAAA |
| | | AUAAUAAAUCUUGGAUGGAAUCUGAAUUUCGCGUUUACUCUUCCGCUAACAACUGCACUUUUGAGUACGUUUCCC |
| | | AGCCUUUUCUGAUGGAUCUGGAAGGCAAACAGGGAAAUUUUAAAAAUCUGAGAGAAUUUGUUUUUAAGAACAUUG |
| | | ACGGAUAUUUCAAGAUCUAUUCUAAACACACUCCCAUCAAUUUGGUUAGAGAUUUACCUCAGGGCUUUUCUGCUU |
| | | UAGAACCUUUAGUUGACCUGCCUAUUGGAAUCAACAUUACUAGAUUUCAAACUCUGUUAGCUCUGCAUAGAUCUU |
| | | AUUUAACUCCUGGAGAUUCCUCCUCCGGCUGGACCGCCGGAGCUGCUGCUUAUUACGUUGGCUAUUUACAACCC |
| | | AGAACUUUUCUGUUAAAAUAUAACGAAAACGGCACCAUCACUGACGCCGUGGAUUGUGCUUUAGAUCCCUUAUCC |
| | | GAAACUAAGUGUACUCUGAAAUCCUUCACUGUUGAAAAAGGAAUUUAUCAGACCUCUAAUUUUAGAGUGCAACCUA |
| | | CCGAAUCUAUUGUUAGAUUUCCUAAUAUUACCAACUUGUGCCCUUUUGGCGAAGUUUUCAACGCUACUAGAUUUG |
| | | CUUCUGUUUACGCUUGGAAUAGAAAAAGAAUUUCUAACUGUGUGGCUGACUAUUCCGUGUUAUACAAUUCUGCCU |
| | | CUUUUUCUACUUUUAAGUGUUACGGAGUGUCUCCCACUAAACUGAACGAUUUGUGCUUUACCAACGUUUACGCUG |
| | | AUUCUUUUGUUAUUAGAGGCGACGAGGUUCGCCAGAUUGCCCCUGGCCAGACUGGCAAAAUUGCUGACUAUAACU |
| | | AUAAAUUACCUGACGAUUUUACCGGCUGCGUUAUCGCUUGGAACUCUAAUAAUCUGGAUUCUAAAGUUGGAGGAA |
| | | ACUACAAUUAUUUAUAUAGAUUAUUUAGAAAAUCUAAUUUAAAACCCUUCGAGAGGGAUAUCUCUACUGAAAUCUA |
| | | CCAAGCUGGAUCUACUCCUUGCAACGGCGUUGAAGGAUUUAAUUGUUAUUUUCCUUUACAAUCUUACGGAUUCCA |
| | | ACCUACUAACGGAGUUGGAUAUCAGCCUUAUAGAGUGGUGGUUUUAUCUUUCGAAUUAUUACACGCUCCCGCUAC |
| | | UGUGUGUGGACCCAAAAAGUCUACUAAUUUAGUUAAAAAUAAGUGUGUUAAUUUCAAUUUUAACGGACUCACUGG |
| | | CACUGGAGUGUUAACUGAAUCUAAUAAAAAGUUCUUACCUUUCCAACAGUUCGGACGCGACAUUGCCGACACCAC |
| | | UGACGCUGUGAGAGAUCCUCAAACCUUAGAGAUUCUGGAUAUUACCCCUUGUUCCUUUGGAGGCGUUUCCGUUA |
| | | UCACUCCCGGAACUAACACUUCUAAUCAGGUUGCCGUUUUAUAUCAGGACGUUAAUUGCACUGAAGUUCCUGUUG |
| | | CUAUUCACGCUGAUCAACUGACUCCUACUUGGAGAGUUUAUUCCACUGGAUCUAACGUUUUUCAGACUCGCGCCG |
| | | GCUGUUUAAUUGGAGCUGAACACGUGAACAACUCCUACGAGUGCGAUAUUCCUAUUGGCGCUGGCAUCUGUGCU |
| | | UCUUAUCAAACUCAAACUAAUUCUCCCAGGAGGGCUCGCUCUGUUGCUUCCCAAUCUAUCAUUGCUUAUACUAUG |
| | | UCCUUAGGCGCUGAAAAUUCUGUUGCUUAUUCCAACAAUUCUAUUGCUAUUCCCACCAAUUUUACUAUUUCUGUG |
| | | ACUACUGAAAUUUUACCUGUUUCCAUGACUAAGACUUCUGUUGAUUGUACUAUGUAUAUCUGCGGAGAUUCUACU |
| | | GAGUGUUCCAACCUCUUAUUACAGUACGGAUCUUUUUGCACUCAGUUAAAUAGAGCUCUGACUGGAAUCGCUGUU |
| | | GAACAAGACAAAAACACCCAAGAAGUGUUUGCUCAGGUGAAGCAAAUUUACAAGACACCUCCUAUUAAGGAUUUUG |
| | | GAGGAUUCAAUUUUUCCCAGAUUUUACCUGAUCCUUCUAAACCUUCUAAACGCUCCUUUAUUGAAGAUUUACUGU |
| | | UCAAUAAAGUGACUCUGGCUGACGCUGGCUUCAUCAAGCAAUACGGCGAUUGUUUAGGAGACAUCGCUGCUAGAG |
| | | ACUUAAUUUGUGCUCAAAAAUUCAACGGACUGACUGUUCUGCCCCCUUUACUGACCGACGAGAUGAUCGCCCAAU |
| | | AUACUUCUGCCCUGUUAGCUGGCACCAUUACUUCUGGCUGGACCUUUGGAGCUGGCGCUGCUUUACAAAUUCCU |
| | | UUCGCCAUGCAGAUGGCUUAUCGCUUUAACGGAAUUGGCGUUACCCAAAACGUGUUAUACGAGAAUCAAAAAUUA |
| | | AUCGCCAAUCAAUUUAAUUCUGCUAUUGGCAAGAUUCAAGAUUCUUUAUCUUCUACUGCUUCCGCCCUGGGCAAA |
| | | CUGCAGGACGUUGUUAACCAGAACGCUCAAGCCCUGAACACUUUAGUGAAGCAACUGUCUUCUAACUUCGGCGCU |
| | | AUUUCUUCCGUUUUAAACGACAUUCUGUCUCGCUUAGAUCCCCCUGAGGCUGAGGUUCAAAUUGAUAGACUGAUU |
| | | ACUGGAAGAUUACAGUCCCUGCAAACUUACGUUACUCAACAGUUAAUCAGAGCUGCUGAAAUCCGCGCUUCCGCU |
| | | AAUUUAGCUGCUACUAAAAUGUCUGAGUGUGUGUUAGGCCAGUCUAAGAGAGUUGACUUUUGUGGCAAGGGCUA |
| | | UCAUUUAAUGUCUUUUCCUCAGUCCGCUCCUCACGGCGUUGUGUUCUUACACGUGACUUACGUUCCCGCCCAAGA |
| | | GAAAAAUUUUACCACUGCUCCUGCUAUUUGUCACGACGGAAAAGCUCAUUUUCCUAGAGAAGGAGUGUUUGUUUC |
| | | UAACGGAACCCACUGGUUUGUGACUCAAAGAAACUUUUACGAACCCCAAAUUAUUACUACUGACAAUACUUUCGUU |
| | | UCCGGCAACUGUGACGUGGUGAUCGGAAUUGUUAAUAACACCGUUUACGAUCCCUUACAACCUGAAUUAGAUUCU |
| | | UUUAAAGAAGAAUUAGAUAAAUAUUUCAAAAAUCAUACUUCCCCUGACGUUGAUCUGGGAGAUAUUUCUGGAAUUA |
| | | ACGCUUCUGUGGUGAACAUUCAAAAAGAGAUUGACAGAUUAAACGAAGUUGCUAAAAACCUGAACGAGUCCCUGAU |
| | | CGACCUGCAGGAACUGGGAAAAUACGAGCAGUAUAUUAAGUGGCCUUGGUAUAUUUGGUUAGGAUUUAUUGCUG |
| | | GAUUAAUUGCUAUUGUGAUGGUGACCAUUAUGUUGUGCUGUAUGACCUCUUGUUGUUCUUGCCUGAAGGGCUGC |
| | | UGUUCUUGUGGCUCUUGUUGCAAGUUUGACGAAGACGAUUCCGAACCCGUUUUAAAGGGAGUGAAACUGCAUUAU |
| | | ACUUAAAUGAGACCAUA |
| 148 | 2A5581 | AUAGGUCUCACACAUUAAAACAGCCUGUGGGUUGAUCCCACCCACAGGCCCAUUGGGCGCUAGCACUCUGGUAUC |
| | | ACGGUACCUUUGUGCGCCUGUUUUAUACCCCCUCCCCCAACUGUAACUUAGAAGUAACACACACCGAUCAACAGU |
| | | CAGCGUGGCACACCAGCCACGUUUUGAUCAAGCACUUCUGUUACCCCGGACUGAGUAUCAAUAGACUGCUCACGC |
| | | GGUUGAAGGAGAAAGCGUUCGUUAUCCGGCCAACUACUUCGAAAAACCUAGUAACACCGUGGAAGUUGCAGAGUG |
| | | UUUCGCUCAGCACUACCCCAGUGUAGAUCAGGUCGAUGAGUCACCGCAUUCCCCACGGGCGACCGUGGCGGUGG |
| | | CUGCGUUGGCGGCCUGCCCAUGGGGAAACCCAUGGGACGCUCUAAUACAGACAUGGUGCGAAGAGUCUAUUGAG |
| | | CUAGUUGGUAGUCCUCCGGCCCCUGAAUGCGGCUAAUCCUAACUGCGGAGCACACACCCUCAAGCCAGAGGGCA |
| | | GUGUGUCGUAACGGGCAACUCUGCAGCGGAACCGACUACUUUGGGUGUCCGUGUUUCAUUUUAUUCCUAUACUG |
| | | GCUGCUUAUGGUGACAAUUGAGAGAUCGUUACCAUAUAGCUAUUGGAUUGGCCAUCCGGUGACUAAUAGAGCUAU |
| | | UAUAUAUCCCUUUGUUGGGUUUAUACCACUUAGCUUGAAAGAGGUUAAAACAUUACAAUUCAUUGUUAAGUUGAAU |
| | | ACAGCAACAUGUUUGUUUUUCUGGUUUUAUUACCCUUAGUGUCCUCCCAGUGCGUGAACUUAACUACCCGCACUC |
| | | AGCUGCCUCCUGCCUAUACCAACUCUUUCACUAGAGGCGUUUACUAUCCUGACAAAGUUUUUAGAUCUUCUGUGC |
| | | UGCAUUCCACUCAAGAUUUAUUCCUGCCCUUCUUUUCUAACGUUACUUGGUUUCACGCCAUCCACGUGUCUGGCA |
| | | CCAACGGAACCAAACGCUUUGACAACCCUGUGUUACCUUUUAACGACGGAGUUUACUUCGCCUCCACUGAAAAGU |
| | | CCAAUAUUAUCCGGGGCUGGAUUUUCGGAACCACCUUAGAUUCUAAAACCCAGUCUCUGCUGAUUGUGAACAACG |
| | | CCACUAACGUGGUUAUUAAAGUGUGCGAGUUUCAAUUUUGCAACGACCCUUUUCUGGGAGUUUAUUACCACAAAA |
| | | AUAACAAAUCUUGGAUGGAGUCUGAAUUUAGAGUGUAUUCUUCCGCCAAUAAUUGCACCUUCGAGUACGUUUCCC |
| | | AACCCUUUCUGAUGGACUUAGAAGGAAAGCAAGGCAAUUUCAAAAACCUGCGCGAAUUUGUGUUCAAAAAUAUUGA |
| | | CGGCUAUUUCAAAAUUUACUCCAAGCAUACCCCUAUUAAUUUAGUGCGCGAUCUGCCUCAGGGCUUCUCUGCCUU |
| | | AGAGCCUUUAGUGGACCUGCCUAUUGGCAUUAAUAUUACUCGCUUUCAGACUUUACUGGCCCUGCAUAGAUCUUA |
| | | UUUAACUCCUGGAGACUCUUCUUCCGGCUGGACCGCUGGAGCCGCUGCCUACUACGUUGGCUAUCUGCAGCCUA |
| | | GAACUUUUUUAUUAAAGUACAACGAAAACGGAACCAUCACUGACGCUGUUGAUUGCGCCCUGGAUCCUUUAUCUG |
| | | AAACAAAGUGCACCCUGAAGUCCUUCACCGUUGAAAAGGGCAUUUACCAAACAUCCAAUUUUAGGGUUCAGCCUAC |
| | | UGAAUCCAUCGUGCGCUUUCCCAACAUCACUAACUUGUGUCCCUUUGGCGAGGUGUUUAACGCCACUCGCUUUG |
| | | CCUCUGUGUACGCCUGGAACAGAAAGAGAAUUUCCAAUUGCGUGGCUGAUUAUUCUGUGCUGUAUAACUCCGCUU |
| | | CUUUCUCCACCUUUAAGUGUUACGGAGUGUCCCCUACUAAACUGAACGACUUGUGCUUCACCAACGUUUACGCCG |
| | | AUUCUUUUGUGAUUCGCGGCGACGAAGUUAGACAAAUCGCCCCCGGACAGACCGGAAAGAUCGCUGAUUACAACU |
| | | AUAAGUUACCUGACGACUUCACCGGCUGCGUGAUUGCUUGGAAUUCUAAUAACUUAGACUCUAAAGUUGGAGGCA |
| | | ACUACAAUUACCUGUAUAGAUUAUUCCGCAAAUCUAACCUGAAACCUUUUGAGAGAGACAUCUCCACCGAAAUUUA |
| | | CCAAGCCGGCUCUACCCCCUGCAACGGAGUUGAAGGAUUUAAUUGCUACUUUCCCCUGCAGUCCUACGGAUUCCA |
| | | ACCUACUAACGGCGUUGGAUACCAACCUUACAGAGUUGUGGUGCUGUCUUUUGAAUUAUUACACGCCCCUGCCAC |
| | | CGUUUGUGGCCCUAAGAAAUCUACCAAUCUGGUUAAAAACAAGUGUGUGAAUUUUAAUUUUAACGGCCUGACCGG |
| | | CACUGGCGUUUUAACCGAGUCUAACAAGAAGUUUCUGCCUUUCCAACAAUUUGGCAGAGACAUCGCCGACACCAC |
| | | CGACGCUGUGCGCGAUCCUCAAACCUUAGAAAUUUUAGAUAUUACUCCUUGCUCUUUCGGCGGCGUUUCCGUUAU |
| | | UACUCCUGGAACCAAUACCUCUAAUCAAGUGGCUGUGCUGUACCAGGACGUUAAUUGCACUGAAGUGCCUGUGGC |
| | | UAUUCACGCCGAUCAACUGACCCCCACUUGGAGAGUUUAUUCUACUGGAUCUAACGUGUUCCAAACUAGAGCUGG |
| | | CUGUCUGAUCGGCGCCGAGCACGUUAAUAAUUCUUACGAGUGUGAUAUCCCUAUUGGCGCUGGCAUUUGCGCCU |
| | | CUUACCAGACCCAGACCAACUCCCCCCGCAGAGCUAGAUCCGUGGCCUCUCAGUCCAUUAUUGCCUAUACUAUGU |
| | | CCUUAGGAGCCGAAAAUUCUGUGGCUUACUCCAACAAUUCUAUUGCCAUUCCCACUAAUUUUACUAUCUCUGUGA |
| | | CUACCGAAAUCCUGCCCGUGUCCAUGACUAAGACCUCCGUGGACUGCACCAUGUAUAUCUGCGGAGACUCUACUG |
| | | AGUGUUCCAACUUAUUACUGCAGUACGGCUCUUUCUGUACUCAGCUGAAUAGAGCCCUGACUGGAAUUGCUGUG |
| | | GAGCAAGACAAAAAUACCCAGGAGGUGUUCGCUCAGGUGAAACAAAUUUAUAAGACCCCUCCCAUCAAAGAUUUUG |
| | | GAGGCUUCAAUUUUUCUCAAAUUCUGCCCGACCCCUCCAAGCCUUCCAAAAGAUCCUUUAUUGAAGAUCUGCUGU |
| | | UCAAUAAGGUUACUUUAGCCGACGCCGGCUUCAUUAAGCAGUACGGAGAUUGUCUGGGCGAUAUUGCUGCCCGC |
| | | GACCUGAUUUGCGCUCAGAAGUUCAACGGAUUAACCGUUCUGCCUCCUCUGCUGACUGACGAGAUGAUUGCUCA |
| | | GUAUACUUCUGCCCUGCUGGCUGGAACUAUUACCUCUGGCUGGACCUUCGGCGCCGGAGCUGCUCUGCAAAUUC |
| | | CCUUCGCCAUGCAAAUGGCUUACAGAUUUAACGGCAUCGGAGUUACUCAAAACGUGCUGUACGAAAACCAAAAAUU |
| | | AAUUGCCAACCAGUUCAAUUCCGCUAUCGGCAAAAUUCAGGAUUCUCUGUCUUCUACUGCUUCCGCUUUAGGCAA |
| | | ACUGCAAGACGUUGUUAACCAGAACGCCCAAGCUUUAAACACCCUGGUUAAGCAGCUGUCCUCUAAUUUUGGCGC |
| | | UAUCUCUUCUGUUCUGAACGACAUUUUAUCUAGACUGGACCCCCCUGAAGCCGAGGUGCAGAUUGAUAGAUUAAU |
| | | CACCGGAAGACUGCAAUCCCUGCAGACCUACGUUACCCAGCAAUUAAUCAGAGCCGCCGAGAUCAGAGCCUCUGC |
| | | CAACUUAGCUGCCACCAAAAUGUCCGAGUGUGUGCUGGGACAGUCCAAGCGCGUGGACUUCUGUGGAAAGGGAU |
| | | ACCACCUGAUGUCUUUUCCUCAAUCUGCUCCCCACGGAGUUGUGUUUCUGCACGUUACUUACGUGCCUGCCCAA |
| | | GAGAAAAACUUUACUACCGCCCCCGCUAUUUGCCACGACGGCAAGGCUCACUUCCCUAGAGAAGGAGUUUUUGUG |
| | | UCUAACGGAACCCAUUGGUUUGUGACUCAAAGAAAUUUUUACGAGCCUCAAAUCAUUACCACCGAUAACACUUUUG |
| | | UUUCCGGCAAUUGUGACGUGGUGAUUGGAAUCGUUAACAACACCGUUUACGACCCUUUACAGCCCGAAUUAGACU |
| | | CUUUUAAAGAGGAGUUAGAUAAAUAUUUUAAAAACCAUACCUCUCCCGACGUGGACCUGGGAGAUAUUUCCGGAA |
| | | UUAACGCUUCUGUUGUGAAUAUCCAGAAAGAGAUCGACAGAUUAAACGAGGUUGCUAAAAAUUUAAACGAAUCCCU |
| | | GAUUGACCUGCAGGAGCUGGGCAAAUACGAACAAUAUAUUAAGUGGCCUUGGUAUAUUUGGUUAGGCUUCAUCGC |
| | | UGGAUUAAUCGCUAUUGUUAUGGUGACUAUUAUGUUGUGUUGUAUGACUUCCUGCUGUUCUUGCUUAAAAGGCU |
| | | GCUGUUCCUGCGGAUCCUGUUGUAAAUUCGACGAGGACGAUUCCGAGCCUGUUUUAAAAGGAGUGAAAUUACAUU |
| | | AUACUUAAAUGAGACCAUA |
| 149 | 2A6581 | AUAGGUCUCACACAUUAAAACAGCCUGUGGGUUGAUCCCACCCACAGGCCCAUUGGGCGCUAGCACUCUGGUAUC |
| | | ACGGUACCUUUGUGCGCCUGUUUUAUACCCCCUCCCCCAACUGUAACUUAGAAGUAACACACACCGAUCAACAGU |
| | | CAGCGUGGCACACCAGCCACGUUUUGAUCAAGCACUUCUGUUACCCCGGACUGAGUAUCAAUAGACUGCUCACGC |
| | | GGUUGAAGGAGAAAGCGUUCGUUAUCCGGCCAACUACUUCGAAAAACCUAGUAACACCGUGGAAGUUGCAGAGUG |
| | | UUUCGCUCAGCACUACCCCAGUGUAGAUCAGGUCGAUGAGUCACCGCAUUCCCCACGGGCGACCGUGGCGGUGG |
| | | CUGCGUUGGCGGCCUGCCCAUGGGGAAACCCAUGGGACGCUCUAAUACAGACAUGGUGCGAAGAGUCUAUUGAG |
| | | CUAGUUGGUAGUCCUCCGGCCCCUGAAUGCGGCUAAUCCUAACUGCGGAGCACACACCCUCAAGCCAGAGGGCA |
| | | GUGUGUCGUAACGGGCAACUCUGCAGCGGAACCGACUACUUUGGGUGUCCGUGUUUCAUUUUAUUCCUAUACUG |
| | | GCUGCUUAUGGUGACAAUUGAGAGAUCGUUACCAUAUAGCUAUUGGAUUGGCCAUCCGGUGACUAAUAGAGCUAU |
| | | UAUAUAUCCCUUUGUUGGGUUUAUACCACUUAGCUUGAAAGAGGUUAAAACAUUACAAUUCAUUGUUAAGUUGAAU |
| | | ACAGCAACAUGUUCGUGUUCCUGGUGCUGCUGCCCUUAGUGUCCUCCCAGUGUGUGAACCUGACCACCAGAACC |
| | | CAGUUACCUCCCGCUUACACCAACUCCUUUACCCGCGGAGUGUACUACCCUGAUAAGGUUUUUAGAUCCUCCGUU |
| | | CUGCAUUCUACCCAAGACCUGUUUUUACCCUUCUUUUCCAACGUGACCUGGUUCCACGCUAUCCACGUGUCCGGC |
| | | ACUAACGGCACUAAGCGCUUCGACAAUCCUGUGCUCCCUUUUAACGACGGCGUGUACUUCGCUUCUACCGAAAAA |
| | | UCCAACAUCAUCCGCGGCUGGAUCUUCGGAACUACCCUGGAUUCUAAAACCCAGUCUUUAUUAAUCGUGAAUAAC |
| | | GCCACCAACGUGGUUAUUAAGGUCUGUGAGUUCCAAUUUUGCAACGACCCUUUCUUAGGCGUUUAUUACCACAAA |
| | | AACAAUAAGUCUUGGAUGGAGUCCGAAUUCCGCGUGUACUCCUCUGCCAACAAUUGCACCUUUGAGUACGUGUCC |
| | | CAGCCCUUCCUGAUGGACCUGGAAGGCAAGCAGGGCAAUUUUAAAAAUCUGCGCGAAUUCGUUUUCAAGAACAUC |
| | | GACGGCUACUUUAAGAUUUAUUCCAAACAUACCCCCAUCAACCUGGUGCGCGAUUUACCCCAGGGCUUCUCCGCC |
| | | UUAGAGCCCUUAGUGGAUCUGCCUAUCGGCAUCAAUAUCACCCGCUUUCAGACUCUGCUGGCCCUGCACAGAUCC |
| | | UACCUGACUCCCGGAGACUCUUCUUCUGGCUGGACCGCCGGAGCUGCCGCCUACUACGUGGGCUACCUGCAACC |
| | | CCGCACCUUUCUGCUGAAGUACAACGAAAACGGCACCAUCACAGACGCCGUUGACUGCGCUCUGGACCCCCUGUC |
| | | UGAGACUAAGUGCACCCUGAAGUCCUUCACUGUGGAGAAAGGAAUCUACCAGACCUCCAACUUCCGCGUGCAGCC |
| | | UACCGAAUCCAUUGUUCGCUUCCCUAACAUUACUAAUCUGUGCCCCUUCGGAGAAGUUUUCAACGCCACUAGAUU |
| | | CGCUUCCGUGUACGCCUGGAAUCGCAAGCGCAUCUCCAAUUGCGUUGCUGACUACUCUGUGUUAUACAAUUCCG |
| | | CCUCUUUUUCCACUUUCAAGUGCUACGGCGUGUCUCCUACCAAGCUGAACGACCUGUGUUUCACUAACGUGUACG |
| | | CUGAUUCCUUUGUUAUCCGCGGCGACGAGGUGAGACAGAUCGCCCCUGGCCAGACCGGAAAAAUCGCCGACUAC |
| | | AACUACAAGUUACCCGACGAUUUUACCGGCUGUGUUAUUGCUUGGAACUCUAAUAAUCUGGACUCUAAAGUGGGC |
| | | GGCAAUUACAACUACUUAUAUCGCCUGUUCAGAAAAUCCAACCUGAAACCCUUCGAACGGGACAUUUCCACCGAAA |
| | | UUUAUCAGGCCGGCUCCACCCCCUGCAACGGAGUGGAGGGCUUCAAUUGCUACUUCCCCCUGCAAUCCUACGGC |
| | | UUUCAGCCCACCAACGGCGUGGGAUACCAGCCCUACCGCGUUGUUGUGUUAUCCUUUGAACUGCUGCACGCUCC |
| | | UGCUACUGUGUGCGGACCUAAAAAAUCCACCAAUCUGGUGAAGAACAAGUGUGUGAAUUUUAAUUUUAACGGCCU |
| | | GACCGGCACCGGAGUGCUGACCGAGUCCAACAAAAAGUUUCUGCCCUUUCAGCAGUUCGGCCGGGACAUUGCCG |
| | | ACACUACCGACGCUGUGAGGGAUCCUCAGACCCUGGAGAUUCUGGACAUUACCCCUUGCUCCUUCGGAGGAGUU |
| | | UCCGUGAUCACCCCUGGCACCAAUACUUCUAACCAAGUGGCUGUUUUAUACCAGGACGUGAAUUGUACUGAGGUG |
| | | CCUGUGGCCAUCCACGCCGAUCAAUUAACUCCCACCUGGAGAGUGUAUUCCACCGGCUCUAACGUUUUCCAAACC |
| | | CGCGCUGGCUGCUUAAUCGGCGCUGAGCACGUGAACAACUCCUACGAGUGUGAUAUUCCUAUCGGAGCCGGCAU |
| | | CUGUGCUUCCUACCAGACUCAGACCAACUCCCCCAGACGCGCUAGGUCCGUGGCUUCCCAGUCUAUUAUCGCUUA |
| | | UACUAUGUCCCUGGGAGCCGAGAAUUCUGUUGCCUAUUCCAAUAACUCCAUCGCCAUUCCUACCAACUUUACCAU |
| | | CUCUGUGACCACCGAGAUUCUGCCCGUGUCCAUGACCAAAACUUCUGUGGAUUGUACCAUGUACAUCUGUGGAGA |
| | | UUCCACCGAGUGCUCUAACCUGCUGCUGCAGUACGGCUCCUUUUGCACCCAAUUAAACCGCGCUCUGACUGGCAU |
| | | CGCCGUGGAGCAAGAUAAAAACACCCAAGAGGUGUUCGCUCAGGUGAAGCAGAUCUACAAGACACCUCCCAUUAA |
| | | AGAUUUCGGAGGCUUUAAUUUCUCUCAGAUCCUGCCCGACCCUUCUAAGCCCUCUAAACGCUCCUUCAUCGAGGA |
| | | UCUGCUGUUCAACAAGGUUACCCUGGCCGACGCUGGCUUUAUCAAACAGUACGGCGACUGCUUAGGCGACAUCG |
| | | CCGCCCGCGAUUUAAUUUGCGCUCAGAAGUUCAACGGACUGACCGUGCUGCCUCCUCUGCUGACCGACGAAAUG |
| | | AUCGCCCAAUACACCUCCGCCCUGCUGGCUGGCACUAUCACCUCCGGCUGGACCUUUGGCGCUGGAGCCGCUCU |
| | | GCAAAUCCCUUUCGCUAUGCAGAUGGCUUACCGCUUUAACGGAAUCGGCGUGACCCAGAACGUGUUAUACGAAAA |
| | | UCAGAAAUUAAUUGCCAAUCAAUUUAACUCUGCCAUCGGCAAGAUUCAGGAUUCUCUGUCUUCUACUGCCUCUGC |
| | | CUUAGGAAAACUGCAGGACGUGGUGAAUCAGAACGCCCAGGCCCUGAAUACCCUGGUUAAGCAACUGUCUUCCAA |
| | | UUUCGGCGCUAUCUCCUCUGUGUUAAACGACAUUCUGUCCCGCCUGGAUCCUCCUGAGGCCGAAGUGCAAAUUG |
| | | ACCGCUUAAUCACUGGCAGACUGCAAUCCCUGCAAACCUACGUUACUCAACAGCUGAUCAGAGCCGCCGAAAUCC |
| | | GCGCUUCUGCCAACUUAGCUGCUACCAAGAUGUCCGAGUGCGUGCUGGGACAGUCCAAAAGAGUUGACUUUUGC |
| | | GGAAAGGGCUACCACCUGAUGUCCUUUCCCCAGUCCGCCCCCCACGGAGUUGUUUUCCUGCACGUUACUUACGU |
| | | UCCUGCUCAAGAGAAGAACUUCACCACCGCUCCCGCUAUUUGCCACGACGGCAAGGCUCAUUUCCCCAGAGAGGG |
| | | AGUGUUUGUGUCCAACGGCACUCACUGGUUUGUUACUCAGCGCAACUUCUACGAACCUCAGAUCAUCACCACUGA |
| | | CAAUACCUUUGUGUCCGGAAACUGUGACGUGGUUAUCGGCAUUGUGAACAAUACUGUGUACGACCCUUUACAACC |
| | | UGAGCUGGAUUCUUUUAAAGAAGAACUGGACAAGUACUUCAAGAACCACACCUCCCCCGACGUGGAUCUGGGCGA |
| | | CAUCUCCGGCAUCAACGCCUCUGUGGUGAAUAUUCAGAAGGAGAUUGAUCGCCUGAACGAGGUUGCCAAGAAUCU |
| | | GAACGAGUCUCUGAUCGACCUGCAGGAGCUGGGCAAGUACGAACAGUACAUCAAGUGGCCCUGGUACAUUUGGC |
| | | UGGGCUUUAUUGCUGGACUGAUCGCUAUCGUUAUGGUUACCAUCAUGCUGUGCUGUAUGACCUCUUGCUGCUCC |
| | | UGUCUGAAAGGCUGUUGCUCCUGCGGCUCCUGUUGUAAGUUUGACGAGGACGACUCCGAGCCUGUUCUGAAAGG |
| | | AGUUAAACUGCACUACACCUAAAUGAGACCAUA |
| 150 | 2A7581 | AUAGGUCUCACACAUUAAAACAGCCUGUGGGUUGAUCCCACCCACAGGCCCAUUGGGCGCUAGCACUCUGGUAUC |
| | | ACGGUACCUUUGUGCGCCUGUUUUAUACCCCCUCCCCCAACUGUAACUUAGAAGUAACACACACCGAUCAACAGU |
| | | CAGCGUGGCACACCAGCCACGUUUUGAUCAAGCACUUCUGUUACCCCGGACUGAGUAUCAAUAGACUGCUCACGC |
| | | GGUUGAAGGAGAAAGCGUUCGUUAUCCGGCCAACUACUUCGAAAAACCUAGUAACACCGUGGAAGUUGCAGAGUG |
| | | UUUCGCUCAGCACUACCCCAGUGUAGAUCAGGUCGAUGAGUCACCGCAUUCCCCACGGGCGACCGUGGCGGUGG |
| | | CUGCGUUGGCGGCCUGCCCAUGGGGAAACCCAUGGGACGCUCUAAUACAGACAUGGUGCGAAGAGUCUAUUGAG |
| | | CUAGUUGGUAGUCCUCCGGCCCCUGAAUGCGGCUAAUCCUAACUGCGGAGCACACACCCUCAAGCCAGAGGGCA |
| | | GUGUGUCGUAACGGGCAACUCUGCAGCGGAACCGACUACUUUGGGUGUCCGUGUUUCAUUUUAUUCCUAUACUG |
| | | GCUGCUUAUGGUGACAAUUGAGAGAUCGUUACCAUAUAGCUAUUGGAUUGGCCAUCCGGUGACUAAUAGAGCUAU |
| | | UAUAUAUCCCUUUGUUGGGUUUAUACCACUUAGCUUGAAAGAGGUUAAAACAUUACAAUUCAUUGUUAAGUUGAAU |
| | | ACAGCAACAUGUUUGUGUUUCUGGUGCUGCUGCCUCUGGUGUCCUCUCAGUGUGUUAACCUGACCACCAGAACC |
| | | CAACUGCCCCCCGCCUACACUAACUCUUUCACUCGCGGCGUGUACUACCCCGAUAAAGUUUUCAGAUCCUCCGUG |
| | | CUCCAUUCUACCCAAGACCUGUUCUUACCCUUUUUCUCCAACGUGACCUGGUUCCACGCCAUCCACGUGUCCGGC |
| | | ACUAACGGAACCAAGCGCUUCGAUAAUCCCGUGCUGCCUUUCAACGACGGAGUUUACUUUGCUUCCACCGAGAAG |
| | | UCCAAUAUUAUCCGGGGCUGGAUCUUCGGCACCACACUGGAUUCCAAAACCCAGUCCUUACUGAUUGUGAACAAC |
| | | GCCACUAACGUGGUGAUCAAGGUGUGUGAGUUCCAGUUCUGUAACGACCCCUUUCUGGGCGUGUAUUAUCAUAA |
| | | AAACAACAAGUCCUGGAUGGAGUCCGAGUUUAGAGUGUAUUCUUCCGCUAACAACUGCACCUUCGAAUACGUGUC |
| | | CCAGCCUUUCCUGAUGGAUCUGGAAGGAAAGCAGGGCAACUUCAAGAACUUACGCGAGUUCGUGUUCAAGAACAU |
| | | CGACGGCUACUUCAAAAUUUACUCCAAGCACACCCCUAUCAACUUAGUGAGAGAUCUGCCCCAGGGCUUCUCCGC |
| | | UCUGGAACCUCUGGUGGACUUACCCAUUGGCAUCAACAUCACUCGCUUCCAAACACUGCUCGCUCUGCACCGCUC |
| | | UUAUCUGACCCCCGGCGACUCCUCUUCUGGCUGGACCGCCGGCGCCGCUGCCUAUUACGUGGGAUACCUGCAAC |
| | | CCCGCACCUUUCUGCUGAAAUACAACGAAAACGGCACCAUCACAGACGCUGUGGACUGCGCUCUGGACCCCCUGU |
| | | CCGAGACUAAGUGCACCCUGAAGUCCUUCACCGUUGAAAAGGGCAUUUAUCAGACUUCCAACUUCCGCGUGCAGC |
| | | CUACUGAGUCCAUUGUGCGCUUCCCUAACAUCACUAACUUGUGUCCUUUCGGCGAAGUGUUCAACGCUACUCGCU |
| | | UCGCCUCCGUGUACGCCUGGAACCGCAAGAGAAUCUCCAAUUGCGUGGCCGACUACUCUGUUCUCUACAACUCUG |
| | | CCUCCUUCUCCACCUUUAAGUGCUACGGCGUGUCCCCCACCAAACUGAACGACCUGUGCUUCACCAACGUGUACG |
| | | CCGACUCCUUUGUGAUCAGAGGCGACGAGGUGCGCCAGAUCGCCCCCGGCCAGACUGGCAAGAUCGCCGAUUAC |
| | | AAUUACAAGCUGCCUGACGACUUUACCGGCUGCGUGAUUGCCUGGAACUCCAACAACCUGGACUCCAAAGUGGGC |
| | | GGCAACUACAACUACCUGUACCGCCUGUUUAGAAAAUCCAACCUGAAGCCCUUCGAGCGGGACAUUUCUACUGAG |
| | | AUCUACCAGGCCGGCUCCACCCCCUGUAACGGCGUGGAAGGCUUCAACUGCUACUUUCCCCUGCAGUCCUACGG |
| | | CUUCCAGCCUACCAACGGCGUGGGCUACCAGCCCUACCGCGUGGUGGUGUUAUCCUUCGAGCUGCUGCACGCCC |
| | | CUGCCACCGUGUGCGGACCCAAAAAGUCUACUAACCUGGUUAAGAAUAAGUGUGUUAAUUUCAAUUUUAACGGCU |
| | | UAACCGGAACCGGAGUGCUGACCGAGUCCAAUAAAAAGUUCCUGCCCUUUCAGCAGUUUGGCCGCGACAUCGCCG |
| | | AUACUACCGACGCCGUGCGCGAUCCCCAGACCCUGGAGAUCCUGGACAUUACCCCCUGCUCUUUCGGCGGAGUG |
| | | UCCGUGAUCACCCCCGGCACCAAUACCUCCAAUCAGGUGGCCGUUCUGUACCAGGACGUGAAUUGCACCGAAGUG |
| | | CCCGUGGCCAUUCACGCCGACCAGUUAACUCCUACUUGGAGAGUGUACUCCACCGGCUCUAACGUGUUUCAGACC |
| | | CGCGCCGGCUGUCUGAUCGGCGCCGAGCACGUGAACAACUCCUACGAGUGCGACAUUCCCAUUGGCGCCGGCAU |
| | | CUGCGCCUCCUACCAAACCCAGACUAACUCUCCCAGACGCGCCCGCUCUGUGGCCUCUCAGUCCAUCAUCGCUUA |
| | | CACUAUGUCCCUGGGCGCCGAGAACUCCGUGGCCUAUUCCAACAACUCCAUCGCCAUCCCCACUAACUUCACCAU |
| | | CUCUGUGACCACCGAAAUCCUCCCCGUGUCUAUGACUAAGACUUCCGUGGACUGUACCAUGUACAUUUGCGGCGA |
| | | CUCCACCGAGUGUUCCAAUCUGCUGCUGCAGUACGGCUCCUUUUGCACCCAGCUGAACCGCGCCCUGACCGGCA |
| | | UCGCUGUUGAGCAGGACAAGAAUACCCAGGAGGUGUUUGCUCAGGUGAAGCAAAUCUAUAAGACCCCUCCCAUCA |
| | | AGGACUUUGGCGGCUUCAACUUUUCCCAGAUCCUGCCCGACCCUUCCAAGCCCUCCAAACGCUCUUUCAUCGAAG |
| | | AUCUGCUGUUCAAUAAAGUGACCCUGGCCGACGCCGGCUUCAUCAAGCAGUACGGAGACUGUCUGGGAGACAUU |
| | | GCUGCUCGCGACCUGAUUUGCGCCCAGAAGUUCAACGGCCUGACCGUGCUGCCCCCUCUGCUGACCGACGAGAU |
| | | GAUCGCUCAGUACACCUCCGCCCUGCUGGCUGGCACUAUCACCUCCGGCUGGACUUUCGGCGCUGGCGCCGCCC |
| | | UGCAGAUUCCCUUCGCCAUGCAGAUGGCUUAUCGCUUCAACGGAAUCGGAGUGACACAGAACGUGCUGUACGAGA |
| | | AUCAGAAACUGAUCGCUAACCAGUUUAAUUCCGCCAUUGGCAAGAUCCAGGACUCCCUGUCCUCUACUGCUUCCG |
| | | CCCUGGGCAAGCUGCAGGACGUGGUGAACCAGAACGCCCAGGCUCUGAACACCCUGGUGAAGCAACUGUCUUCU |
| | | AACUUUGGAGCCAUUUCUUCCGUGCUGAACGACAUCCUGUCCAGACUGGAUCCUCCUGAGGCCGAGGUGCAGAU |
| | | CGACCGCCUGAUCACUGGCCGCCUGCAGUCCCUGCAGACUUACGUUACCCAGCAAUUAAUCCGCGCUGCCGAGA |
| | | UCCGCGCCUCCGCCAACCUGGCCGCCACCAAGAUGUCCGAGUGCGUUCUGGGCCAGUCCAAACGCGUUGACUUC |
| | | UGCGGCAAGGGCUACCACCUGAUGUCCUUCCCCCAGUCCGCUCCCCACGGAGUGGUGUUCCUGCACGUGACCUA |
| | | CGUGCCUGCCCAGGAGAAGAACUUCACCACCGCCCCCGCCAUUUGCCACGACGGAAAGGCCCACUUCCCCCGCGA |
| | | GGGAGUGUUCGUUUCUAACGGCACUCACUGGUUCGUGACCCAGAGAAACUUCUACGAGCCCCAGAUUAUUACCAC |
| | | CGACAAUACCUUCGUUUCUGGCAACUGCGACGUGGUUAUCGGCAUCGUGAACAACACCGUGUACGACCCCCUGCA |
| | | ACCCGAGCUGGAUUCUUUCAAGGAGGAGCUGGAUAAGUACUUUAAAAACCAUACCUCCCCCGACGUGGACCUGGG |
| | | CGACAUCUCUGGCAUCAACGCCUCCGUGGUGAAUAUCCAGAAGGAGAUCGAUCGCCUGAACGAGGUGGCCAAGAA |
| | | CUUAAACGAAUCUCUGAUCGACCUGCAGGAGCUGGGCAAGUACGAGCAGUAUAUUAAGUGGCCCUGGUAUAUCUG |
| | | GUUAGGCUUUAUCGCCGGCCUGAUCGCCAUUGUUAUGGUGACCAUCAUGCUGUGCUGCAUGACCUCCUGCUGUU |
| | | CCUGUUUAAAAGGCUGCUGCUCCUGCGGAUCCUGCUGUAAAUUCGACGAAGACGACUCCGAGCCCGUGCUGAAA |
| | | GGAGUUAAGUUACACUAUACCUAAAUGAGACCAUA |
| 151 | 2A8581 | AUAGGUCUCACACAUUAAAACAGCCUGUGGGUUGAUCCCACCCACAGGCCCAUUGGGCGCUAGCACUCUGGUAUC |
| | | ACGGUACCUUUGUGCGCCUGUUUUAUACCCCCUCCCCCAACUGUAACUUAGAAGUAACACACACCGAUCAACAGU |
| | | CAGCGUGGCACACCAGCCACGUUUUGAUCAAGCACUUCUGUUACCCCGGACUGAGUAUCAAUAGACUGCUCACGC |
| | | GGUUGAAGGAGAAAGCGUUCGUUAUCCGGCCAACUACUUCGAAAAACCUAGUAACACCGUGGAAGUUGCAGAGUG |
| | | UUUCGCUCAGCACUACCCCAGUGUAGAUCAGGUCGAUGAGUCACCGCAUUCCCCACGGGCGACCGUGGCGGUGG |
| | | CUGCGUUGGCGGCCUGCCCAUGGGGAAACCCAUGGGACGCUCUAAUACAGACAUGGUGCGAAGAGUCUAUUGAG |
| | | CUAGUUGGUAGUCCUCCGGCCCCUGAAUGCGGCUAAUCCUAACUGCGGAGCACACACCCUCAAGCCAGAGGGCA |
| | | GUGUGUCGUAACGGGCAACUCUGCAGCGGAACCGACUACUUUGGGUGUCCGUGUUUCAUUUUAUUCCUAUACUG |
| | | GCUGCUUAUGGUGACAAUUGAGAGAUCGUUACCAUAUAGCUAUUGGAUUGGCCAUCCGGUGACUAAUAGAGCUAU |
| | | UAUAUAUCCCUUUGUUGGGUUUAUACCACUUAGCUUGAAAGAGGUUAAAACAUUACAAUUCAUUGUUAAGUUGAAU |
| | | ACAGCAACAUGUUCGUGUUCCUGGUGCUGCUGCCCCUGGUGUCCUCCCAGUGCGUCAACCUGACCACCCGGACC |
| | | CAACUGCCCCCUGCCUAUACCAAUUCCUUCACCCGGGGCGUCUACUACCCCGACAAGGUGUUCCGGUCCUCUGU |
| | | GCUCCACUCCACCCAGGACCUCUUCCUCCCCUUUUUCUCCAACGUGACCUGGUUCCACGCCAUCCACGUGUCUG |
| | | GCACAAACGGAACCAAGCGCUUCGACAACCCUGUGCUCCCCUUCAACGACGGCGUGUACUUCGCCUCCACCGAGA |
| | | AGUCCAACAUCAUCCGCGGCUGGAUCUUCGGCACCACCCUCGACUCUAAGACCCAGUCCCUGCUGAUUGUGAAUA |
| | | ACGCCACCAACGUGGUGAUCAAGGUGUGCGAAUUUCAGUUCUGCAACGACCCCUUCCUGGGCGUUUACUACCAUA |
| | | AGAACAACAAGUCCUGGAUGGAGUCCGAGUUCCGGGUGUAUUCCUCUGCCAAUAACUGCACCUUUGAGUACGUGU |
| | | CCCAGCCCUUCCUGAUGGACCUGGAGGGCAAGCAGGGCAAUUUUAAAAACCUGCGCGAGUUCGUGUUCAAGAACA |
| | | UCGACGGCUACUUCAAGAUCUAUUCCAAGCACACCCCUAUCAACCUGGUGCGCGACCUGCCCCAGGGCUUUUCCG |
| | | CUCUGGAGCCCCUGGUUGACCUGCCCAUCGGCAUCAACAUCACCCGCUUCCAGACUUUACUGGCCCUGCACCGC |
| | | UCCUACCUGACCCCCGGCGAUUCCUCUUCUGGCUGGACAGCCGGAGCCGCUGCCUACUACGUGGGAUACCUGCA |
| | | GCCCCGCACCUUCCUGCUGAAGUACAACGAGAACGGAACCAUUACAGACGCUGUCGACUGUGCUCUGGAUCCCCU |
| | | GUCCGAAACAAAGUGCACCCUGAAGUCCUUCACCGUGGAGAAGGGCAUCUACCAGACCUCCAACUUCCGCGUGCA |
| | | GCCCACCGAGUCCAUCGUGAGAUUCCCCAACAUCACUAACCUGUGCCCCUUCGGCGAGGUGUUCAACGCCACCCG |
| | | CUUCGCCUCCGUGUACGCCUGGAACCGCAAGCGCAUCUCCAACUGCGUUGCCGACUACUCUGUGCUCUACAACU |
| | | CCGCCUCCUUCUCCACCUUCAAGUGCUACGGCGUGUCCCCCACCAAGCUGAACGACCUGUGCUUCACCAACGUGU |
| | | ACGCCGACUCCUUCGUGAUCCGCGGCGACGAGGUGCGCCAGAUCGCCCCCGGCCAGACCGGAAAGAUCGCCGAU |
| | | UACAAUUACAAGCUGCCCGACGACUUCACCGGCUGCGUGAUCGCCUGGAACUCCAACAAUCUGGACUCCAAGGUU |
| | | GGCGGCAACUACAACUACCUGUACCGCCUGUUCCGCAAGUCCAACCUGAAGCCCUUCGAGCGGGACAUUUCCACA |
| | | GAGAUUUACCAGGCUGGCUCCACCCCCUGUAACGGAGUGGAGGGCUUCAACUGCUACUUCCCCCUGCAGUCCUA |
| | | CGGCUUCCAGCCCACCAACGGCGUGGGCUACCAGCCCUACCGCGUGGUGGUGCUGUCCUUCGAGCUGCUGCACG |
| | | CCCCUGCCACCGUGUGCGGCCCCAAGAAGUCCACCAACCUGGUGAAGAACAAGUGCGUGAACUUCAACUUCAACG |
| | | GACUGACAGGCACCGGCGUGCUGACCGAGUCCAACAAGAAGUUCCUGCCCUUCCAGCAGUUCGGCCGGGACAUU |
| | | GCCGACACCACCGACGCCGUGCGCGACCCCCAGACCCUGGAGAUCCUGGACAUCACCCCCUGCUCCUUCGGCGG |
| | | AGUGUCCGUGAUCACCCCCGGCACCAACACCUCCAACCAGGUUGCCGUGCUGUACCAGGACGUUAACUGCACCGA |
| | | GGUGCCCGUGGCCAUCCACGCCGACCAGCUGACCCCCACCUGGCGCGUGUACUCCACCGGCUCCAACGUGUUCC |
| | | AGACCCGCGCCGGCUGCCUGAUCGGCGCCGAGCACGUGAACAACUCCUACGAGUGCGACAUCCCCAUUGGCGCC |
| | | GGCAUCUGCGCCUCCUACCAGACCCAGACCAACUCCCCUCGCCGCGCCCGGUCCGUCGCCUCCCAGUCCAUCAU |
| | | CGCCUACACCAUGUCCCUGGGCGCCGAGAACUCCGUGGCCUACUCCAACAACUCCAUUGCCAUCCCCACCAACUU |
| | | CACCAUCUCCGUGACCACCGAAAUCCUCCCCGUGUCCAUGACCAAGACCUCCGUGGACUGCACCAUGUACAUCUG |
| | | CGGCGACUCCACCGAGUGCUCCAACCUGCUGCUGCAGUACGGCUCCUUCUGCACCCAGCUGAAUCGCGCCCUGA |
| | | CCGGCAUCGCCGUGGAGCAGGACAAGAACACCCAAGAGGUGUUCGCCCAGGUGAAGCAGAUCUACAAGACACCUC |
| | | CCAUCAAGGACUUCGGCGGCUUCAACUUCUCCCAGAUCCUGCCCGACCCCUCCAAGCCCUCCAAGCGCUCCUUCA |
| | | UCGAGGACCUGCUGUUCAACAAGGUGACCCUGGCCGACGCCGGCUUCAUCAAGCAGUACGGCGACUGUCUGGGA |
| | | GACAUUGCCGCCCGCGACCUGAUCUGCGCCCAGAAGUUCAACGGCUUAACCGUGCUGCCCCCUCUGCUGACCGA |
| | | CGAGAUGAUCGCCCAGUACACAUCCGCUCUGCUGGCCGGCACCAUCACCUCCGGCUGGACCUUUGGCGCUGGCG |
| | | CUGCCCUGCAGAUCCCCUUCGCUAUGCAGAUGGCCUACCGCUUCAACGGCAUCGGCGUGACCCAGAACGUGCUG |
| | | UACGAGAACCAGAAGCUGAUCGCCAACCAGUUCAACUCCGCCAUCGGCAAGAUCCAGGACUCCCUGUCCUCCACC |
| | | GCCUCCGCCCUGGGCAAGCUGCAGGACGUGGUGAACCAGAACGCCCAGGCCCUGAACACCCUGGUGAAGCAGCU |
| | | GUCCUCUAACUUCGGCGCCAUCUCCUCCGUGCUGAACGACAUUCUGUCCCGCCUGGAUCCUCCUGAGGCCGAGG |
| | | UGCAAAUCGAUCGCCUCAUCACCGGCCGCCUGCAGUCCCUGCAGACCUACGUGACACAGCAGCUGAUCCGCGCC |
| | | GCCGAGAUCCGCGCCUCCGCCAACCUGGCCGCCACCAAGAUGUCUGAGUGCGUGCUGGGCCAGUCCAAACGCGU |
| | | UGACUUCUGCGGCAAGGGCUACCACCUGAUGUCCUUCCCCCAGUCCGCCCCCCACGGCGUGGUGUUCCUGCACG |
| | | UGACCUACGUGCCCGCCCAGGAGAAGAACUUCACUACCGCCCCCGCCAUCUGCCACGACGGCAAGGCCCACUUCC |
| | | CCCGCGAGGGCGUGUUCGUGUCCAACGGCACCCACUGGUUCGUGACCCAGCGCAACUUCUACGAGCCCCAGAUC |
| | | AUCACCACCGACAACACCUUCGUGUCCGGCAACUGCGACGUGGUGAUUGGCAUCGUGAACAACACCGUGUACGAC |
| | | CCCCUGCAGCCCGAGCUGGACUCCUUCAAGGAGGAGCUGGACAAGUACUUUAAGAACCACACCUCCCCCGACGUG |
| | | GACCUGGGCGACAUCUCCGGCAUCAACGCCUCCGUGGUGAACAUCCAGAAGGAGAUCGACCGCCUGAACGAGGU |
| | | GGCCAAGAACCUGAACGAGUCCCUGAUCGACCUGCAGGAGCUGGGCAAGUACGAACAGUACAUCAAGUGGCCCUG |
| | | GUACAUCUGGCUGGGCUUCAUCGCCGGCCUGAUCGCCAUCGUGAUGGUGACCAUCAUGCUGUGCUGCAUGACCU |
| | | CUUGCUGUUCCUGUCUGAAGGGCUGCUGCUCCUGCGGCUCCUGCUGCAAGUUCGACGAGGACGACUCCGAGCCC |
| | | GUGCUGAAGGGCGUGAAGCUGCACUACACCUAAAUGAGACCAUA |
| 152 | 2A9581 | AUAGGUCUCACACAUUAAAACAGCCUGUGGGUUGAUCCCACCCACAGGCCCAUUGGGCGCUAGCACUCUGGUAUC |
| | | ACGGUACCUUUGUGCGCCUGUUUUAUACCCCCUCCCCCAACUGUAACUUAGAAGUAACACACACCGAUCAACAGU |
| | | CAGCGUGGCACACCAGCCACGUUUUGAUCAAGCACUUCUGUUACCCCGGACUGAGUAUCAAUAGACUGCUCACGC |
| | | GGUUGAAGGAGAAAGCGUUCGUUAUCCGGCCAACUACUUCGAAAAACCUAGUAACACCGUGGAAGUUGCAGAGUG |
| | | UUUCGCUCAGCACUACCCCAGUGUAGAUCAGGUCGAUGAGUCACCGCAUUCCCCACGGGCGACCGUGGCGGUGG |
| | | CUGCGUUGGCGGCCUGCCCAUGGGGAAACCCAUGGGACGCUCUAAUACAGACAUGGUGCGAAGAGUCUAUUGAG |
| | | CUAGUUGGUAGUCCUCCGGCCCCUGAAUGCGGCUAAUCCUAACUGCGGAGCACACACCCUCAAGCCAGAGGGCA |
| | | GUGUGUCGUAACGGGCAACUCUGCAGCGGAACCGACUACUUUGGGUGUCCGUGUUUCAUUUUAUUCCUAUACUG |
| | | GCUGCUUAUGGUGACAAUUGAGAGAUCGUUACCAUAUAGCUAUUGGAUUGGCCAUCCGGUGACUAAUAGAGCUAU |
| | | UAUAUAUCCCUUUGUUGGGUUUAUACCACUUAGCUUGAAAGAGGUUAAAACAUUACAAUUCAUUGUUAAGUUGAAU |
| | | ACAGCAACAUGUUCGUGUUCCUGGUGCUGCUCCCCCUGGUGAGCUCCCAGUGCGUCAACCUGACCACACGGACC |
| | | CAGCUGCCCCCCGCCUAUACCAACAGCUUCACCCGCGGCGUCUACUACCCCGACAAGGUGUUCCGGAGCAGCGU |
| | | GCUGCACAGCACCCAGGACCUCUUCCUCCCCUUUUUUAGCAACGUGACCUGGUUCCACGCCAUCCACGUGAGUG |
| | | GCACCAACGGCACCAAGCGGUUCGACAACCCUGUGCUCCCCUUCAACGACGGCGUGUACUUCGCUAGCACAGAAA |
| | | AAAGUAACAUCAUUCGGGGCUGGAUCUUCGGCACCACACUGGAUAGCAAAACCCAGAGCCUGCUGAUUGUGAAUA |
| | | ACGCCACCAACGUGGUGAUCAAGGUGUGCGAGUUCCAGUUCUGCAACGACCCCUUCCUGGGCGUGUACUACCAC |
| | | AAGAACAACAAGAGCUGGAUGGAGAGCGAGUUCCGCGUGUAUAGCUCUGCCAACAACUGCACCUUCGAGUACGUG |
| | | AGCCAGCCCUUCCUGAUGGACCUGGAGGGCAAGCAGGGCAAUUUCAAAAACCUGCGGGAGUUCGUGUUCAAGAA |
| | | CAUCGACGGCUACUUCAAGAUCUACAGCAAGCACACCCCCAUCAACCUGGUGCGGGACCUGCCCCAGGGCUUUAG |
| | | CGCUCUGGAGCCCCUGGUGGACCUGCCCAUCGGCAUCAACAUCACCCGCUUCCAAACCCUGUUGGCCCUGCACC |
| | | GGAGCUACCUGACCCCUGGCGAUAGCAGUAGUGGCUGGACAGCCGGAGCCGCCGCCUACUACGUGGGAUACCUG |
| | | CAGCCCCGGACCUUCCUGCUGAAGUACAACGAGAACGGCACAAUCACAGACGCCGUGGACUGCGCCCUGGACCCC |
| | | CUGAGCGAAACAAAGUGCACCCUCAAAAGUUUCACCGUGGAGAAGGGCAUCUACCAGACCAGCAACUUCCGGGUG |
| | | CAGCCCACCGAGAGCAUCGUGCGGUUCCCCAACAUCACCAACCUGUGCCCCUUCGGCGAGGUGUUCAACGCCAC |
| | | CCGGUUCGCCAGCGUGUACGCCUGGAACCGGAAGCGGAUCAGCAACUGCGUGGCCGACUACAGCGUGCUGUAUA |
| | | ACAGUGCCAGCUUCAGCACCUUCAAGUGCUACGGCGUGAGCCCCACCAAGCUGAACGACCUGUGCUUCACCAACG |
| | | UGUACGCCGACAGCUUCGUGAUCCGGGGCGACGAGGUGCGGCAGAUCGCCCCCGGCCAGACCGGCAAGAUCGCC |
| | | GAUUACAAUUACAAGCUGCCCGACGAUUUCACCGGCUGCGUGAUCGCCUGGAACAGCAACAACCUGGACAGCAAG |
| | | GUGGGCGGCAACUACAACUACCUGUACCGGCUGUUCCGCAAAAGUAACCUGAAGCCCUUCGAGCGGGACAUCAGC |
| | | ACCGAGAUCUACCAGGCCGGCAGUACCCCUUGCAACGGCGUCGAGGGCUUCAACUGCUACUUCCCCCUGCAGAG |
| | | CUACGGCUUCCAGCCCACCAACGGCGUGGGCUACCAGCCCUACCGGGUGGUGGUUCUGAGCUUCGAGCUGCUGC |
| | | ACGCCCCUGCCACCGUGUGCGGCCCCAAGAAAAGCACCAACCUGGUGAAGAACAAGUGCGUGAAUUUCAAUUUUA |
| | | ACGGACUCACCGGAACCGGCGUGCUGACCGAGAGCAACAAGAAGUUCCUGCCCUUCCAGCAAUUCGGCCGGGAU |
| | | AUUGCCGACACCACUGACGCCGUGCGGGACCCCCAGACCCUGGAGAUCCUGGACAUCACACCCUGUAGCUUCGG |
| | | CGGGGUGAGCGUGAUCACCCCCGGCACCAACACCAGCAACCAGGUGGCCGUGCUGUACCAGGACGUGAACUGCA |
| | | CCGAGGUGCCCGUGGCCAUCCACGCCGACCAGCUGACCCCCACCUGGGGGGUGUACAGCACCGGCAGCAACGUG |
| | | UUCCAGACCCGGGCCGGCUGCCUGAUCGGCGCCGAGCACGUGAAUAACAGCUACGAGUGCGACAUCCCCAUCGG |
| | | AGCCGGAAUCUGCGCCAGCUACCAGACCCAGACCAACAGCCCCCGGAGAGCCCGGAGCGUGGCCAGCCAGAGCA |
| | | UUAUUGCCUACACCAUGAGCCUGGGCGCCGAGAACAGCGUGGCCUACAGCAACAAUAGUAUUGCCAUCCCCACCA |
| | | ACUUCACCAUCAGCGUGACCACCGAAAUCCUCCCCGUGAGCAUGACCAAGACCAGCGUGGACUGCACCAUGUACA |
| | | UCUGCGGCGACAGCACCGAGUGCAGCAACCUGCUCCUGCAGUACGGCAGCUUCUGCACCCAGCUGAACCGGGCC |
| | | CUGACCGGAAUUGCCGUGGAGCAGGACAAGAACACCCAGGAGGUGUUCGCCCAGGUGAAGCAGAUCUACAAGACA |
| | | CCUCCCAUCAAGGACUUCGGCGGCUUCAACUUCAGCCAGAUCCUGCCCGACCCCAGCAAGCCCAGCAAGCGGAGC |
| | | UUCAUCGAGGACCUGUUGUUCAACAAGGUGACCCUGGCCGACGCCGGCUUCAUCAAGCAGUACGGCGACUGUCU |
| | | GGGAGAUAUUGCCGCACGGGACCUGAUCUGCGCCCAGAAGUUCAACGGCCUGACCGUGCUGCCCCCACUGCUGA |
| | | CCGACGAGAUGAUCGCCCAGUACACAAGCGCUCUGCUCGCCGGCACCAUCACCAGCGGCUGGACCUUCGGCGCC |
| | | GGCGCUGCCCUGCAGAUCCCCUUCGCCAUGCAGAUGGCCUACCGGUUCAACGGCAUCGGCGUGACCCAGAACGU |
| | | GCUGUACGAGAACCAGAAGCUGAUCGCCAACCAGUUCAACAGCGCCAUCGGCAAGAUCCAGGACAGCCUGAGCUC |
| | | CACCGCCAGCGCCCUGGGCAAGCUGCAGGACGUGGUGAACCAGAACGCCCAGGCCCUGAACACCCUGGUGAAGC |
| | | AGCUGAGCAGCAACUUCGGCGCCAUCAGCUCUGUGCUGAACGACAUCCUGAGCCGGCUGGAUCCUCCUGAGGCC |
| | | GAGGUGCAAAUCGAUCGGCUCAUCACCGGCCGGCUGCAGAGCCUGCAGACCUACGUGACACAGCAGCUGAUCCG |
| | | GGCCGCCGAGAUCCGGGCCAGCGCCAACCUGGCCGCUACCAAGAUGAGCGAGUGCGUGCUGGGCCAGAGCAAGC |
| | | GGGUGGACUUCUGCGGCAAGGGCUACCACCUGAUGAGCUUCCCCCAGAGCGCCCCCCACGGCGUGGUGUUCCUG |
| | | CACGUGACCUACGUGCCCGCCCAGGAGAAGAACUUCACCACCGCCCCCGCCAUCUGCCACGACGGCAAGGCCCAC |
| | | UUCCCCCGGGAGGGCGUGUUCGUGAGCAACGGCACCCACUGGUUCGUGACCCAGCGGAACUUCUACGAGCCCCA |
| | | GAUCAUAACCACAGACAACACCUUCGUGAGCGGCAACUGCGACGUGGUCAUCGGCAUCGUGAACAACACCGUGUA |
| | | CGACCCCCUGCAGCCCGAGCUGGACAGCUUCAAGGAGGAACUGGACAAGUACUUCAAGAACCACACCAGCCCCGA |
| | | CGUGGACCUGGGCGACAUCAGCGGCAUCAACGCCAGCGUGGUGAACAUCCAGAAGGAGAUCGACCGGCUGAACG |
| | | AGGUGGCCAAGAACCUGAACGAGAGCCUGAUCGACCUGCAGGAGCUGGGCAAGUACGAGCAGUACAUCAAGUGG |
| | | CCCUGGUACAUCUGGCUGGGCUUUAUUGCCGGCCUGAUCGCCAUCGUGAUGGUGACCAUCAUGCUGUGCUGUAU |
| | | GACAAGCUGUUGCAGUUGCCUGAAGGGCUGCUGCAGCUGCGGCAGCUGCUGCAAGUUCGACGAGGACGACAGCG |
| | | AGCCCGUGCUGAAGGGCGUGAAGCUGCACUACACCUAAAUGAGACCAUA |
| 153 | 2A0681 | AUAGGUCUCACACACUAGGGCGCGCCAGUCCUCAAGCUGGGCCUGACACAACUGUGUUCACUAGCAACUACUCCC |
| | | AGACACCAUGGUACUCCCGACUCCUGAGGUACUCCCUGCCGUUACUGCCCUGUGGGGCAAGGUGAUACUCCCUG |
| | | AAGUUGGUGGUGAGGCCCUGGGCAGGCUACUCCCGGUCUACCCUUGGACCCAGAGGUCGACUCGGACCGGCCAC |
| | | CAUGUUUGUUUUCUUAGUUUUAUUACCUUUAGUUUCUUCCCAGUGUGUUAAUUUAACUACUAGAACUCAAUUACC |
| | | UCCUGCUUAUACUAAUUCUUUUACUAGAGGAGUGUAUUAUCCUGAUAAGGUUUUUAGAUCUUCUGUUCUGCACUC |
| | | UACUCAGGAUUUGUUCUUGCCCUUCUUCUCUAACGUGACCUGGUUCCACGCUAUUCACGUUUCUGGAACCAACGG |
| | | CACUAAGAGAUUUGAUAAUCCCGUUUUGCCUUUUAACGACGGAGUUUAUUUCGCCUCUACCGAAAAAUCUAACAU |
| | | CAUUAGGGGCUGGAUCUUUGGAACUACCCUGGAUUCUAAAACUCAAUCUUUAUUGAUUGUGAAUAACGCUACCAA |
| | | CGUUGUGAUUAAAGUUUGUGAAUUUCAAUUUUGUAACGAUCCUUUUCUGGGAGUUUAUUAUCAUAAAAACAAUAAA |
| | | UCUUGGAUGGAAUCUGAAUUCCGCGUUUAUUCUUCUGCCAAUAACUGUACCUUUGAGUACGUUUCUCAACCUUUC |
| | | UUAAUGGAUUUAGAAGGAAAACAAGGCAAUUUCAAAAACUUAAGAGAAUUUGUUUUUAAAAAUAUUGACGGAUAUU |
| | | UUAAAAUUUAUUCUAAGCAUACUCCUAUUAAUUUAGUGAGAGACUUACCCCAAGGAUUUUCUGCUUUAGAACCUUU |
| | | AGUUGACUUACCUAUUGGAAUCAAUAUCACUAGAUUUCAAACUUUAUUAGCUUUACAUAGAUCUUAUUUAACUCCU |
| | | GGAGAUUCUUCCUCCGGCUGGACAGCCGGAGCCGCUGCUUAUUACGUUGGAUAUUUACAGCCUAGAACUUUUCU |
| | | GCUGAAAUAUAACGAAAACGGAACCAUUACCGACGCCGUUGACUGUGCUCUGGAUCCUUUAUCUGAAACCAAGUG |
| | | UACUUUAAAAUCUUUUACCGUUGAAAAAGGAAUUUAUCAAACUUCUAAUUUUAGAGUUCAACCUACUGAAUCUAUC |
| | | GUUAGAUUCCCUAAUAUUACUAAUUUGUGUCCCUUUGGAGAGGUUUUCAACGCUACUAGAUUUGCCUCUGUUUAC |
| | | GCUUGGAAUCGCAAGAGAAUUUCUAAUUGUGUGGCCGAUUACUCCGUUUUGUAUAACUCUGCUUCUUUCUCUACU |
| | | UUUAAGUGUUACGGAGUUUCCCCUACUAAACUGAACGAUUUGUGUUUUACUAACGUUUACGCUGAUUCUUUUGUU |
| | | AUUAGAGGCGACGAAGUUAGACAAAUUGCUCCUGGACAAACUGGAAAGAUUGCUGACUAUAACUAUAAACUGCCU |
| | | GACGAUUUUACCGGCUGUGUUAUUGCUUGGAAUUCUAAUAAUUUAGAUUCUAAAGUUGGAGGAAAUUAUAAUUAU |
| | | UUAUAUAGAUUAUUUAGAAAAUCUAAUUUAAAACCUUUUGAAAGAGAUAUUUCCACUGAGAUUUACCAGGCUGGAU |
| | | CUACUCCUUGUAACGGCGUUGAGGGAUUUAAUUGUUAUUUUCCUUUACAAUCUUACGGAUUUCAACCUACCAACG |
| | | GAGUUGGAUAUCAACCUUACAGAGUUGUGGUGCUGUCUUUUGAAUUAUUACACGCUCCCGCUACUGUUUGCGGC |
| | | CCUAAAAAGUCUACUAAUCUGGUUAAGAAUAAGUGUGUGAAUUUUAAUUUCAACGGAUUGACCGGAACUGGAGUU |
| | | CUGACUGAAUCUAAUAAAAAAUUUUUACCCUUUCAACAAUUUGGAAGGGAUAUCGCUGAUACUACUGACGCUGUUA |
| | | GAGAUCCUCAAACUUUAGAAAUCCUGGAUAUUACUCCUUGUUCUUUUGGAGGAGUUUCCGUUAUUACUCCUGGCA |
| | | CUAAUACUUCUAAUCAAGUUGCUGUUUUAUAUCAAGACGUUAAUUGCACCGAAGUUCCUGUUGCUAUUCACGCUG |
| | | AUCAGUUAACUCCUACUUGGAGAGUUUAUUCUACUGGAUCUAACGUUUUUCAAACCAGAGCUGGCUGUUUAAUCG |
| | | GAGCUGAACACGUGAAUAAUUCUUACGAGUGCGAUAUUCCCAUCGGAGCUGGAAUUUGUGCCUCUUAUCAAACUC |
| | | AAACCAAUUCUCCUAGGAGGGCUAGAUCCGUUGCCUCUCAAUCUAUUAUUGCUUAUACCAUGUCUUUAGGAGCUG |
| | | AAAAUUCCGUUGCUUACUCUAACAAUUCUAUUGCUAUUCCUACUAACUUUACUAUUUCUGUUACUACUGAAAUUCU |
| | | GCCCGUGUCCAUGACUAAAACUUCUGUUGAUUGUACUAUGUAUAUUUGUGGAGAUUCCACUGAGUGUUCUAACCU |
| | | GUUAUUACAAUACGGAUCUUUUUGUACUCAAUUAAAUAGAGCUCUGACUGGAAUUGCUGUUGAACAGGACAAAAAU |
| | | ACUCAAGAAGUUUUUGCCCAAGUUAAACAGAUUUAUAAAACUCCUCCUAUUAAAGAUUUUGGAGGAUUUAAUUUCU |
| | | CUCAAAUUUUACCUGACCCUUCUAAGCCUUCCAAAAGGUCUUUCAUCGAAGAUUUAUUAUUUAAUAAAGUUACCUU |
| | | AGCCGACGCUGGAUUUAUUAAGCAAUACGGAGAUUGCUUAGGCGAUAUCGCUGCUAGAGACUUAAUUUGUGCUCA |
| | | AAAAUUUAACGGAUUAACUGUUUUACCUCCCCUGUUAACUGACGAAAUGAUCGCUCAAUAUACUUCUGCUCUGUUA |
| | | GCUGGAACUAUUACUUCUGGCUGGACUUUUGGAGCUGGAGCUGCUUUACAAAUUCCCUUUGCUAUGCAGAUGGC |
| | | UUAUAGAUUUAACGGAAUUGGAGUUACUCAAAACGUUUUAUACGAAAAUCAAAAAUUAAUUGCUAAUCAAUUUAAU |
| | | UCUGCUAUUGGAAAAAUUCAAGAUUCCCUGUCUUCUACUGCUUCCGCUCUGGGAAAAUUACAAGACGUUGUUAAU |
| | | CAAAACGCUCAAGCUUUGAAUACCUUAGUUAAACAAUUAUCUUCCAAUUUUGGAGCUAUUUCUUCUGUUUUAAACG |
| | | ACAUUUUAUCUAGAUUAGAUCCCCCUGAAGCUGAAGUUCAGAUUGACAGAUUGAUUACCGGAAGAUUACAAUCUU |
| | | UACAAACUUACGUUACUCAACAACUGAUUAGAGCUGCUGAAAUUAGAGCCUCUGCUAAUUUAGCUGCUACUAAGAU |
| | | GUCUGAGUGUGUUUUAGGACAGUCCAAAAGAGUUGAUUUUUGUGGAAAAGGCUAUCAUCUGAUGUCUUUUCCUCA |
| | | AUCUGCUCCUCACGGAGUUGUGUUUUUACACGUUACUUACGUUCCUGCUCAAGAGAAGAAUUUUACUACUGCUCC |
| | | UGCUAUUUGUCACGACGGAAAAGCUCAUUUUCCUAGAGAAGGAGUUUUUGUUUCUAACGGAACUCAUUGGUUUGU |
| | | UACUCAAAGAAACUUUUACGAACCUCAGAUUAUUACCACUGAUAAUACCUUUGUUUCUGGCAAUUGUGACGUGGU |
| | | UAUUGGAAUUGUUAAUAAUACUGUUUACGAUCCUUUACAACCCGAACUGGAUUCUUUUAAAGAAGAAUUAGAUAAA |
| | | UACUUUAAAAAUCAUACUUCUCCCGACGUUGAUUUAGGAGAUAUCUCUGGAAUUAACGCUUCUGUUGUGAAUAUU |
| | | CAAAAGGAAAUUGAUAGAUUAAACGAAGUGGCUAAAAAUUUAAACGAGUCUUUAAUUGAUUUACAGGAACUGGGAA |
| | | AAUACGAACAAUAUAUUAAGUGGCCUUGGUACAUUUGGUUAGGCUUUAUUGCUGGACUGAUUGCUAUUGUGAUGG |
| | | UUACUAUCAUGUUGUGUUGCAUGACUUCUUGUUGUUCCUGUUUAAAAGGCUGUUGCUCUUGUGGAUCUUGUUGC |
| | | AAAUUUGACGAAGACGAUUCUGAACCUGUUCUGAAAGGAGUUAAACUGCAUUAUACUUAAAUGAGACCAUA |
| 154 | 2A1681 | AUAGGUCUCACACACUAGGGCGCGCCAGUCCUCAAGCUGGGCCUGACACAACUGUGUUCACUAGCAACUACUCCC |
| | | AGACACCAUGGUACUCCCGACUCCUGAGGUACUCCCUGCCGUUACUGCCCUGUGGGGCAAGGUGAUACUCCCUG |
| | | AAGUUGGUGGUGAGGCCCUGGGCAGGCUACUCCCGGUCUACCCUUGGACCCAGAGGUCGACUCGGACCGGCCAC |
| | | CAUGUUCGUGUUCUUAGUGCUGUUACCCCUGGUUUCUUCUCAGUGUGUCAAUUUAACCACCAGAACCCAACUGCC |
| | | UCCUGCCUAUACUAAUUCCUUUACCCGCGGAGUUUACUAUCCCGAUAAGGUUUUUCGCUCCUCUGUUUUACACUC |
| | | UACUCAGGACUUAUUCUUACCCUUCUUCUCCAACGUUACUUGGUUUCACGCCAUUCACGUUUCUGGAACCAACGG |
| | | AACUAAGAGAUUCGAUAAUCCUGUGUUACCUUUCAACGACGGAGUUUAUUUUGCUUCCACUGAAAAAUCCAAUAUC |
| | | AUUAGGGGCUGGAUCUUUGGAACCACUUUAGAUUCCAAGACUCAAUCUUUAUUAAUUGUGAAUAACGCCACCAAC |
| | | GUGGUUAUCAAAGUGUGUGAAUUCCAAUUUUGUAACGACCCUUUCUUAGGCGUGUAUUAUCACAAAAAUAAUAAAU |
| | | CUUGGAUGGAAUCUGAAUUUCGCGUUUACUCUUCCGCUAACAACUGCACUUUUGAGUACGUUUCCCAGCCUUUUC |
| | | UGAUGGAUCUGGAAGGCAAACAGGGAAAUUUUAAAAAUCUGAGAGAAUUUGUUUUUAAGAACAUUGACGGAUAUU |
| | | UCAAGAUCUAUUCUAAACACACUCCCAUCAAUUUGGUUAGAGAUUUACCUCAGGGCUUUUCUGCUUUAGAACCUU |
| | | UAGUUGACCUGCCUAUUGGAAUCAACAUUACUAGAUUUCAAACUCUGUUAGCUCUGCAUAGAUCUUAUUUAACUC |
| | | CUGGAGAUUCCUCCUCCGGCUGGACCGCCGGAGCUGCUGCUUAUUACGUUGGCUAUUUACAACCCAGAACUUUU |
| | | CUGUUAAAAUAUAACGAAAACGGCACCAUCACUGACGCCGUGGAUUGUGCUUUAGAUCCCUUAUCCGAAACUAAG |
| | | UGUACUCUGAAAUCCUUCACUGUUGAAAAAGGAAUUUAUCAGACCUCUAAUUUUAGAGUGCAACCUACCGAAUCUA |
| | | UUGUUAGAUUUCCUAAUAUUACCAACUUGUGCCCUUUUGGCGAAGUUUUCAACGCUACUAGAUUUGCUUCUGUUU |
| | | ACGCUUGGAAUAGAAAAAGAAUUUCUAACUGUGUGGCUGACUAUUCCGUGUUAUACAAUUCUGCCUCUUUUUCUA |
| | | CUUUUAAGUGUUACGGAGUGUCUCCCACUAAACUGAACGAUUUGUGCUUUACCAACGUUUACGCUGAUUCUUUUG |
| | | UUAUUAGAGGCGACGAGGUUCGCCAGAUUGCCCCUGGCCAGACUGGCAAAAUUGCUGACUAUAACUAUAAAUUAC |
| | | CUGACGAUUUUACCGGCUGCGUUAUCGCUUGGAACUCUAAUAAUCUGGAUUCUAAAGUUGGAGGAAACUACAAUU |
| | | AUUUAUAUAGAUUAUUUAGAAAAUCUAAUUUAAAACCCUUCGAGAGGGAUAUCUCUACUGAAAUCUACCAAGCUGG |
| | | AUCUACUCCUUGCAACGGCGUUGAAGGAUUUAAUUGUUAUUUUCCUUUACAAUCUUACGGAUUCCAACCUACUAA |
| | | CGGAGUUGGAUAUCAGCCUUAUAGAGUGGUGGUUUUAUCUUUCGAAUUAUUACACGCUCCCGCUACUGUGUGUG |
| | | GACCCAAAAAGUCUACUAAUUUAGUUAAAAAUAAGUGUGUUAAUUUCAAUUUUAACGGACUCACUGGCACUGGAGU |
| | | GUUAACUGAAUCUAAUAAAAAGUUCUUACCUUUCCAACAGUUCGGACGCGACAUUGCCGACACCACUGACGCUGU |
| | | GAGAGAUCCUCAAACCUUAGAGAUUCUGGAUAUUACCCCUUGUUCCUUUGGAGGCGUUUCCGUUAUCACUCCCGG |
| | | AACUAACACUUCUAAUCAGGUUGCCGUUUUAUAUCAGGACGUUAAUUGCACUGAAGUUCCUGUUGCUAUUCACGC |
| | | UGAUCAACUGACUCCUACUUGGAGAGUUUAUUCCACUGGAUCUAACGUUUUUCAGACUCGCGCCGGCUGUUUAAU |
| | | UGGAGCUGAACACGUGAACAACUCCUACGAGUGCGAUAUUCCUAUUGGCGCUGGCAUCUGUGCUUCUUAUCAAAC |
| | | UCAAACUAAUUCUCCCAGGAGGGCUCGCUCUGUUGCUUCCCAAUCUAUCAUUGCUUAUACUAUGUCCUUAGGCGC |
| | | UGAAAAUUCUGUUGCUUAUUCCAACAAUUCUAUUGCUAUUCCCACCAAUUUUACUAUUUCUGUGACUACUGAAAUU |
| | | UUACCUGUUUCCAUGACUAAGACUUCUGUUGAUUGUACUAUGUAUAUCUGCGGAGAUUCUACUGAGUGUUCCAAC |
| | | CUCUUAUUACAGUACGGAUCUUUUUGCACUCAGUUAAAUAGAGCUCUGACUGGAAUCGCUGUUGAACAAGACAAA |
| | | AACACCCAAGAAGUGUUUGCUCAGGUGAAGCAAAUUUACAAGACACCUCCUAUUAAGGAUUUUGGAGGAUUCAAU |
| | | UUUUCCCAGAUUUUACCUGAUCCUUCUAAACCUUCUAAACGCUCCUUUAUUGAAGAUUUACUGUUCAAUAAAGUGA |
| | | CUCUGGCUGACGCUGGCUUCAUCAAGCAAUACGGCGAUUGUUUAGGAGACAUCGCUGCUAGAGACUUAAUUUGU |
| | | GCUCAAAAAUUCAACGGACUGACUGUUCUGCCCCCUUUACUGACCGACGAGAUGAUCGCCCAAUAUACUUCUGCC |
| | | CUGUUAGCUGGCACCAUUACUUCUGGCUGGACCUUUGGAGCUGGCGCUGCUUUACAAAUUCCUUUCGCCAUGCA |
| | | GAUGGCUUAUCGCUUUAACGGAAUUGGCGUUACCCAAAACGUGUUAUACGAGAAUCAAAAAUUAAUCGCCAAUCAA |
| | | UUUAAUUCUGCUAUUGGCAAGAUUCAAGAUUCUUUAUCUUCUACUGCUUCCGCCCUGGGCAAACUGCAGGACGUU |
| | | GUUAACCAGAACGCUCAAGCCCUGAACACUUUAGUGAAGCAACUGUCUUCUAACUUCGGCGCUAUUUCUUCCGUU |
| | | UUAAACGACAUUCUGUCUCGCUUAGAUCCCCCUGAGGCUGAGGUUCAAAUUGAUAGACUGAUUACUGGAAGAUUA |
| | | CAGUCCCUGCAAACUUACGUUACUCAACAGUUAAUCAGAGCUGCUGAAAUCCGCGCUUCCGCUAAUUUAGCUGCU |
| | | ACUAAAAUGUCUGAGUGUGUGUUAGGCCAGUCUAAGAGAGUUGACUUUUGUGGCAAGGGCUAUCAUUUAAUGUCU |
| | | UUUCCUCAGUCCGCUCCUCACGGCGUUGUGUUCUUACACGUGACUUACGUUCCCGCCCAAGAGAAAAAUUUUACC |
| | | ACUGCUCCUGCUAUUUGUCACGACGGAAAAGCUCAUUUUCCUAGAGAAGGAGUGUUUGUUUCUAACGGAACCCAC |
| | | UGGUUUGUGACUCAAAGAAACUUUUACGAACCCCAAAUUAUUACUACUGACAAUACUUUCGUUUCCGGCAACUGU |
| | | GACGUGGUGAUCGGAAUUGUUAAUAACACCGUUUACGAUCCCUUACAACCUGAAUUAGAUUCUUUUAAAGAAGAA |
| | | UUAGAUAAAUAUUUCAAAAAUCAUACUUCCCCUGACGUUGAUCUGGGAGAUAUUUCUGGAAUUAACGCUUCUGUG |
| | | GUGAACAUUCAAAAAGAGAUUGACAGAUUAAACGAAGUUGCUAAAAACCUGAACGAGUCCCUGAUCGACCUGCAGG |
| | | AACUGGGAAAAUACGAGCAGUAUAUUAAGUGGCCUUGGUAUAUUUGGUUAGGAUUUAUUGCUGGAUUAAUUGCUA |
| | | UUGUGAUGGUGACCAUUAUGUUGUGCUGUAUGACCUCUUGUUGUUCUUGCCUGAAGGGCUGCUGUUCUUGUGGC |
| | | UCUUGUUGCAAGUUUGACGAAGACGAUUCCGAACCCGUUUUAAAGGGAGUGAAACUGCAUUAUACUUAAAUGAGA |
| | | CCAUA |
| 155 | 2A2681 | AUAGGUCUCACACACUAGGGCGCGCCAGUCCUCAAGCUGGGCCUGACACAACUGUGUUCACUAGCAACUACUCCC |
| | | AGACACCAUGGUACUCCCGACUCCUGAGGUACUCCCUGCCGUUACUGCCCUGUGGGGCAAGGUGAUACUCCCUG |
| | | AAGUUGGUGGUGAGGCCCUGGGCAGGCUACUCCCGGUCUACCCUUGGACCCAGAGGUCGACUCGGACCGGCCAC |
| | | CAUGUUUGUUUUUCUGGUUUUAUUACCCUUAGUGUCCUCCCAGUGCGUGAACUUAACUACCCGCACUCAGCUGCC |
| | | UCCUGCCUAUACCAACUCUUUCACUAGAGGCGUUUACUAUCCUGACAAAGUUUUUAGAUCUUCUGUGCUGCAUUC |
| | | CACUCAAGAUUUAUUCCUGCCCUUCUUUUCUAACGUUACUUGGUUUCACGCCAUCCACGUGUCUGGCACCAACGG |
| | | AACCAAACGCUUUGACAACCCUGUGUUACCUUUUAACGACGGAGUUUACUUCGCCUCCACUGAAAAGUCCAAUAU |
| | | UAUCCGGGGCUGGAUUUUCGGAACCACCUUAGAUUCUAAAACCCAGUCUCUGCUGAUUGUGAACAACGCCACUAA |
| | | CGUGGUUAUUAAAGUGUGCGAGUUUCAAUUUUGCAACGACCCUUUUCUGGGAGUUUAUUACCACAAAAAUAACAA |
| | | AUCUUGGAUGGAGUCUGAAUUUAGAGUGUAUUCUUCCGCCAAUAAUUGCACCUUCGAGUACGUUUCCCAACCCUU |
| | | UCUGAUGGACUUAGAAGGAAAGCAAGGCAAUUUCAAAAACCUGCGCGAAUUUGUGUUCAAAAAUAUUGACGGCUA |
| | | UUUCAAAAUUUACUCCAAGCAUACCCCUAUUAAUUUAGUGCGCGAUCUGCCUCAGGGCUUCUCUGCCUUAGAGCC |
| | | UUUAGUGGACCUGCCUAUUGGCAUUAAUAUUACUCGCUUUCAGACUUUACUGGCCCUGCAUAGAUCUUAUUUAAC |
| | | UCCUGGAGACUCUUCUUCCGGCUGGACCGCUGGAGCCGCUGCCUACUACGUUGGCUAUCUGCAGCCUAGAACUU |
| | | UUUUAUUAAAGUACAACGAAAACGGAACCAUCACUGACGCUGUUGAUUGCGCCCUGGAUCCUUUAUCUGAAACAAA |
| | | GUGCACCCUGAAGUCCUUCACCGUUGAAAAGGGCAUUUACCAAACAUCCAAUUUUAGGGUUCAGCCUACUGAAUC |
| | | CAUCGUGCGCUUUCCCAACAUCACUAACUUGUGUCCCUUUGGCGAGGUGUUUAACGCCACUCGCUUUGCCUCUG |
| | | UGUACGCCUGGAACAGAAAGAGAAUUUCCAAUUGCGUGGCUGAUUAUUCUGUGCUGUAUAACUCCGCUUCUUUCU |
| | | CCACCUUUAAGUGUUACGGAGUGUCCCCUACUAAACUGAACGACUUGUGCUUCACCAACGUUUACGCCGAUUCUU |
| | | UUGUGAUUCGCGGCGACGAAGUUAGACAAAUCGCCCCCGGACAGACCGGAAAGAUCGCUGAUUACAACUAUAAGU |
| | | UACCUGACGACUUCACCGGCUGCGUGAUUGCUUGGAAUUCUAAUAACUUAGACUCUAAAGUUGGAGGCAACUACA |
| | | AUUACCUGUAUAGAUUAUUCCGCAAAUCUAACCUGAAACCUUUUGAGAGAGACAUCUCCACCGAAAUUUACCAAGC |
| | | CGGCUCUACCCCCUGCAACGGAGUUGAAGGAUUUAAUUGCUACUUUCCCCUGCAGUCCUACGGAUUCCAACCUAC |
| | | UAACGGCGUUGGAUACCAACCUUACAGAGUUGUGGUGCUGUCUUUUGAAUUAUUACACGCCCCUGCCACCGUUU |
| | | GUGGCCCUAAGAAAUCUACCAAUCUGGUUAAAAACAAGUGUGUGAAUUUUAAUUUUAACGGCCUGACCGGCACUG |
| | | GCGUUUUAACCGAGUCUAACAAGAAGUUUCUGCCUUUCCAACAAUUUGGCAGAGACAUCGCCGACACCACCGACG |
| | | CUGUGCGCGAUCCUCAAACCUUAGAAAUUUUAGAUAUUACUCCUUGCUCUUUCGGCGGCGUUUCCGUUAUUACUC |
| | | CUGGAACCAAUACCUCUAAUCAAGUGGCUGUGCUGUACCAGGACGUUAAUUGCACUGAAGUGCCUGUGGCUAUUC |
| | | ACGCCGAUCAACUGACCCCCACUUGGAGAGUUUAUUCUACUGGAUCUAACGUGUUCCAAACUAGAGCUGGCUGUC |
| | | UGAUCGGCGCCGAGCACGUUAAUAAUUCUUACGAGUGUGAUAUCCCUAUUGGCGCUGGCAUUUGCGCCUCUUAC |
| | | CAGACCCAGACCAACUCCCCCCGCAGAGCUAGAUCCGUGGCCUCUCAGUCCAUUAUUGCCUAUACUAUGUCCUUA |
| | | GGAGCCGAAAAUUCUGUGGCUUACUCCAACAAUUCUAUUGCCAUUCCCACUAAUUUUACUAUCUCUGUGACUACC |
| | | GAAAUCCUGCCCGUGUCCAUGACUAAGACCUCCGUGGACUGCACCAUGUAUAUCUGCGGAGACUCUACUGAGUGU |
| | | UCCAACUUAUUACUGCAGUACGGCUCUUUCUGUACUCAGCUGAAUAGAGCCCUGACUGGAAUUGCUGUGGAGCAA |
| | | GACAAAAAUACCCAGGAGGUGUUCGCUCAGGUGAAACAAAUUUAUAAGACCCCUCCCAUCAAAGAUUUUGGAGGC |
| | | UUCAAUUUUUCUCAAAUUCUGCCCGACCCCUCCAAGCCUUCCAAAAGAUCCUUUAUUGAAGAUCUGCUGUUCAAUA |
| | | AGGUUACUUUAGCCGACGCCGGCUUCAUUAAGCAGUACGGAGAUUGUCUGGGCGAUAUUGCUGCCCGCGACCUG |
| | | AUUUGCGCUCAGAAGUUCAACGGAUUAACCGUUCUGCCUCCUCUGCUGACUGACGAGAUGAUUGCUCAGUAUACU |
| | | UCUGCCCUGCUGGCUGGAACUAUUACCUCUGGCUGGACCUUCGGCGCCGGAGCUGCUCUGCAAAUUCCCUUCGC |
| | | CAUGCAAAUGGCUUACAGAUUUAACGGCAUCGGAGUUACUCAAAACGUGCUGUACGAAAACCAAAAAUUAAUUGCC |
| | | AACCAGUUCAAUUCCGCUAUCGGCAAAAUUCAGGAUUCUCUGUCUUCUACUGCUUCCGCUUUAGGCAAACUGCAA |
| | | GACGUUGUUAACCAGAACGCCCAAGCUUUAAACACCCUGGUUAAGCAGCUGUCCUCUAAUUUUGGCGCUAUCUCU |
| | | UCUGUUCUGAACGACAUUUUAUCUAGACUGGACCCCCCUGAAGCCGAGGUGCAGAUUGAUAGAUUAAUCACCGGA |
| | | AGACUGCAAUCCCUGCAGACCUACGUUACCCAGCAAUUAAUCAGAGCCGCCGAGAUCAGAGCCUCUGCCAACUUA |
| | | GCUGCCACCAAAAUGUCCGAGUGUGUGCUGGGACAGUCCAAGCGCGUGGACUUCUGUGGAAAGGGAUACCACCU |
| | | GAUGUCUUUUCCUCAAUCUGCUCCCCACGGAGUUGUGUUUCUGCACGUUACUUACGUGCCUGCCCAAGAGAAAAA |
| | | CUUUACUACCGCCCCCGCUAUUUGCCACGACGGCAAGGCUCACUUCCCUAGAGAAGGAGUUUUUGUGUCUAACG |
| | | GAACCCAUUGGUUUGUGACUCAAAGAAAUUUUUACGAGCCUCAAAUCAUUACCACCGAUAACACUUUUGUUUCCG |
| | | GCAAUUGUGACGUGGUGAUUGGAAUCGUUAACAACACCGUUUACGACCCUUUACAGCCCGAAUUAGACUCUUUUA |
| | | AAGAGGAGUUAGAUAAAUAUUUUAAAAACCAUACCUCUCCCGACGUGGACCUGGGAGAUAUUUCCGGAAUUAACG |
| | | CUUCUGUUGUGAAUAUCCAGAAAGAGAUCGACAGAUUAAACGAGGUUGCUAAAAAUUUAAACGAAUCCCUGAUUGA |
| | | CCUGCAGGAGCUGGGCAAAUACGAACAAUAUAUUAAGUGGCCUUGGUAUAUUUGGUUAGGCUUCAUCGCUGGAUU |
| | | AAUCGCUAUUGUUAUGGUGACUAUUAUGUUGUGUUGUAUGACUUCCUGCUGUUCUUGCUUAAAAGGCUGCUGUU |
| | | CCUGCGGAUCCUGUUGUAAAUUCGACGAGGACGAUUCCGAGCCUGUUUUAAAAGGAGUGAAAUUACAUUAUACUU |
| | | AAAUGAGACCAUA |
| 156 | 2A3681 | AUAGGUCUCACACACUAGGGCGCGCCAGUCCUCAAGCUGGGCCUGACACAACUGUGUUCACUAGCAACUACUCCC |
| | | AGACACCAUGGUACUCCCGACUCCUGAGGUACUCCCUGCCGUUACUGCCCUGUGGGGCAAGGUGAUACUCCCUG |
| | | AAGUUGGUGGUGAGGCCCUGGGCAGGCUACUCCCGGUCUACCCUUGGACCCAGAGGUCGACUCGGACCGGCCAC |
| | | CAUGUUCGUGUUCCUGGUGCUGCUGCCCUUAGUGUCCUCCCAGUGUGUGAACCUGACCACCAGAACCCAGUUAC |
| | | CUCCCGCUUACACCAACUCCUUUACCCGCGGAGUGUACUACCCUGAUAAGGUUUUUAGAUCCUCCGUUCUGCAUU |
| | | CUACCCAAGACCUGUUUUUACCCUUCUUUUCCAACGUGACCUGGUUCCACGCUAUCCACGUGUCCGGCACUAACG |
| | | GCACUAAGCGCUUCGACAAUCCUGUGCUCCCUUUUAACGACGGCGUGUACUUCGCUUCUACCGAAAAAUCCAACA |
| | | UCAUCCGCGGCUGGAUCUUCGGAACUACCCUGGAUUCUAAAACCCAGUCUUUAUUAAUCGUGAAUAACGCCACCA |
| | | ACGUGGUUAUUAAGGUCUGUGAGUUCCAAUUUUGCAACGACCCUUUCUUAGGCGUUUAUUACCACAAAAACAAUA |
| | | AGUCUUGGAUGGAGUCCGAAUUCCGCGUGUACUCCUCUGCCAACAAUUGCACCUUUGAGUACGUGUCCCAGCCC |
| | | UUCCUGAUGGACCUGGAAGGCAAGCAGGGCAAUUUUAAAAAUCUGCGCGAAUUCGUUUUCAAGAACAUCGACGGC |
| | | UACUUUAAGAUUUAUUCCAAACAUACCCCCAUCAACCUGGUGCGCGAUUUACCCCAGGGCUUCUCCGCCUUAGAG |
| | | CCCUUAGUGGAUCUGCCUAUCGGCAUCAAUAUCACCCGCUUUCAGACUCUGCUGGCCCUGCACAGAUCCUACCUG |
| | | ACUCCCGGAGACUCUUCUUCUGGCUGGACCGCCGGAGCUGCCGCCUACUACGUGGGCUACCUGCAACCCCGCAC |
| | | CUUUCUGCUGAAGUACAACGAAAACGGCACCAUCACAGACGCCGUUGACUGCGCUCUGGACCCCCUGUCUGAGAC |
| | | UAAGUGCACCCUGAAGUCCUUCACUGUGGAGAAAGGAAUCUACCAGACCUCCAACUUCCGCGUGCAGCCUACCGA |
| | | AUCCAUUGUUCGCUUCCCUAACAUUACUAAUCUGUGCCCCUUCGGAGAAGUUUUCAACGCCACUAGAUUCGCUUC |
| | | CGUGUACGCCUGGAAUCGCAAGCGCAUCUCCAAUUGCGUUGCUGACUACUCUGUGUUAUACAAUUCCGCCUCUUU |
| | | UUCCACUUUCAAGUGCUACGGCGUGUCUCCUACCAAGCUGAACGACCUGUGUUUCACUAACGUGUACGCUGAUUC |
| | | CUUUGUUAUCCGCGGCGACGAGGUGAGACAGAUCGCCCCUGGCCAGACCGGAAAAAUCGCCGACUACAACUACAA |
| | | GUUACCCGACGAUUUUACCGGCUGUGUUAUUGCUUGGAACUCUAAUAAUCUGGACUCUAAAGUGGGGGCAAUUA |
| | | CAACUACUUAUAUCGCCUGUUCAGAAAAUCCAACCUGAAACCCUUCGAACGGGACAUUUCCACCGAAAUUUAUCAG |
| | | GCCGGCUCCACCCCCUGCAACGGAGUGGAGGGCUUCAAUUGCUACUUCCCCCUGCAAUCCUACGGCUUUCAGCC |
| | | CACCAACGGCGUGGGAUACCAGCCCUACCGCGUUGUUGUGUUAUCCUUUGAACUGCUGCACGCUCCUGCUACUG |
| | | UGUGCGGACCUAAAAAAUCCACCAAUCUGGUGAAGAACAAGUGUGUGAAUUUUAAUUUUAACGGCCUGACCGGCA |
| | | CCGGAGUGCUGACCGAGUCCAACAAAAAGUUUCUGCCCUUUCAGCAGUUCGGCCGGGACAUUGCCGACACUACC |
| | | GACGCUGUGAGGGAUCCUCAGACCCUGGAGAUUCUGGACAUUACCCCUUGCUCCUUCGGAGGAGUUUCCGUGAU |
| | | CACCCCUGGCACCAAUACUUCUAACCAAGUGGCUGUUUUAUACCAGGACGUGAAUUGUACUGAGGUGCCUGUGGC |
| | | CAUCCACGCCGAUCAAUUAACUCCCACCUGGAGAGUGUAUUCCACCGGCUCUAACGUUUUCCAAACCCGCGCUGG |
| | | CUGCUUAAUCGGCGCUGAGCACGUGAACAACUCCUACGAGUGUGAUAUUCCUAUCGGAGCCGGCAUCUGUGCUU |
| | | CCUACCAGACUCAGACCAACUCCCCCAGACGCGCUAGGUCCGUGGCUUCCCAGUCUAUUAUCGCUUAUACUAUGU |
| | | CCCUGGGAGCCGAGAAUUCUGUUGCCUAUUCCAAUAACUCCAUCGCCAUUCCUACCAACUUUACCAUCUCUGUGA |
| | | CCACCGAGAUUCUGCCCGUGUCCAUGACCAAAACUUCUGUGGAUUGUACCAUGUACAUCUGUGGAGAUUCCACCG |
| | | AGUGCUCUAACCUGCUGCUGCAGUACGGCUCCUUUUGCACCCAAUUAAACCGCGCUCUGACUGGCAUCGCCGUG |
| | | GAGCAAGAUAAAAACACCCAAGAGGUGUUCGCUCAGGUGAAGCAGAUCUACAAGACACCUCCCAUUAAAGAUUUCG |
| | | GAGGCUUUAAUUUCUCUCAGAUCCUGCCCGACCCUUCUAAGCCCUCUAAACGCUCCUUCAUCGAGGAUCUGCUGU |
| | | UCAACAAGGUUACCCUGGCCGACGCUGGCUUUAUCAAACAGUACGGCGACUGCUUAGGCGACAUCGCCGCCCGC |
| | | GAUUUAAUUUGCGCUCAGAAGUUCAACGGACUGACCGUGCUGCCUCCUCUGCUGACCGACGAAAUGAUCGCCCAA |
| | | UACACCUCCGCCCUGCUGGCUGGCACUAUCACCUCCGGCUGGACCUUUGGCGCUGGAGCCGCUCUGCAAAUCCC |
| | | UUUCGCUAUGCAGAUGGCUUACCGCUUUAACGGAAUCGGCGUGACCCAGAACGUGUUAUACGAAAAUCAGAAAUU |
| | | AAUUGCCAAUCAAUUUAACUCUGCCAUCGGCAAGAUUCAGGAUUCUCUGUCUUCUACUGCCUCUGCCUUAGGAAA |
| | | ACUGCAGGACGUGGUGAAUCAGAACGCCCAGGCCCUGAAUACCCUGGUUAAGCAACUGUCUUCCAAUUUCGGCGC |
| | | UAUCUCCUCUGUGUUAAACGACAUUCUGUCCCGCCUGGAUCCUCCUGAGGCCGAAGUGCAAAUUGACCGCUUAAU |
| | | CACUGGCAGACUGCAAUCCCUGCAAACCUACGUUACUCAACAGCUGAUCAGAGCCGCCGAAAUCCGCGCUUCUGC |
| | | CAACUUAGCUGCUACCAAGAUGUCCGAGUGCGUGCUGGGACAGUCCAAAAGAGUUGACUUUUGCGGAAAGGGCU |
| | | ACCACCUGAUGUCCUUUCCCCAGUCCGCCCCCCACGGAGUUGUUUUCCUGCACGUUACUUACGUUCCUGCUCAA |
| | | GAGAAGAACUUCACCACCGCUCCCGCUAUUUGCCACGACGGCAAGGCUCAUUUCCCCAGAGAGGGAGUGUUUGU |
| | | GUCCAACGGCACUCACUGGUUUGUUACUCAGCGCAACUUCUACGAACCUCAGAUCAUCACCACUGACAAUACCUU |
| | | UGUGUCCGGAAACUGUGACGUGGUUAUCGGCAUUGUGAACAAUACUGUGUACGACCCUUUACAACCUGAGCUGG |
| | | AUUCUUUUAAAGAAGAACUGGACAAGUACUUCAAGAACCACACCUCCCCCGACGUGGAUCUGGGCGACAUCUCCG |
| | | GCAUCAACGCCUCUGUGGUGAAUAUUCAGAAGGAGAUUGAUCGCCUGAACGAGGUUGCCAAGAAUCUGAACGAGU |
| | | CUCUGAUCGACCUGCAGGAGCUGGGCAAGUACGAACAGUACAUCAAGUGGCCCUGGUACAUUUGGCUGGGCUUU |
| | | AUUGCUGGACUGAUCGCUAUCGUUAUGGUUACCAUCAUGCUGUGCUGUAUGACCUCUUGCUGCUCCUGUCUGAA |
| | | AGGCUGUUGCUCCUGCGGCUCCUGUUGUAAGUUUGACGAGGACGACUCCGAGCCUGUUCUGAAAGGAGUUAAAC |
| | | UGCACUACACCUAAAUGAGACCAUA |
| 157 | 2A4681 | AUAGGUCUCACACACUAGGGCGCGCCAGUCCUCAAGCUGGGCCUGACACAACUGUGUUCACUAGCAACUACUCCC |
| | | AGACACCAUGGUACUCCCGACUCCUGAGGUACUCCCUGCCGUUACUGCCCUGUGGGGCAAGGUGAUACUCCCUG |
| | | AAGUUGGUGGUGAGGCCCUGGGCAGGCUACUCCCGGUCUACCCUUGGACCCAGAGGUCGACUCGGACCGGCCAC |
| | | CAUGUUUGUGUUUCUGGUGCUGCUGCCUCUGGUGUCCUCUCAGUGUGUUAACCUGACCACCAGAACCCAACUGC |
| | | CCCCCGCCUACACUAACUCUUUCACUCGCGGCGUGUACUACCCCGAUAAAGUUUUCAGAUCCUCCGUGCUCCAUU |
| | | CUACCCAAGACCUGUUCUUACCCUUUUUCUCCAACGUGACCUGGUUCCACGCCAUCCACGUGUCCGGCACUAACG |
| | | GAACCAAGCGCUUCGAUAAUCCCGUGCUGCCUUUCAACGACGGAGUUUACUUUGCUUCCACCGAGAAGUCCAAUA |
| | | UUAUCCGGGGCUGGAUCUUCGGCACCACACUGGAUUCCAAAACCCAGUCCUUACUGAUUGUGAACAACGCCACUA |
| | | ACGUGGUGAUCAAGGUGUGUGAGUUCCAGUUCUGUAACGACCCCUUUCUGGGCGUGUAUUAUCAUAAAAACAACA |
| | | AGUCCUGGAUGGAGUCCGAGUUUAGAGUGUAUUCUUCCGCUAACAACUGCACCUUCGAAUACGUGUCCCAGCCUU |
| | | UCCUGAUGGAUCUGGAAGGAAAGCAGGGCAACUUCAAGAACUUACGCGAGUUCGUGUUCAAGAACAUCGACGGCU |
| | | ACUUCAAAAUUUACUCCAAGCACACCCCUAUCAACUUAGUGAGAGAUCUGCCCCAGGGCUUCUCCGCUCUGGAAC |
| | | CUCUGGUGGACUUACCCAUUGGCAUCAACAUCACUCGCUUCCAAACACUGCUCGCUCUGCACCGCUCUUAUCUGA |
| | | CCCCCGGCGACUCCUCUUCUGGCUGGACCGCCGGCGCCGCUGCCUAUUACGUGGGAUACCUGCAACCCCGCACC |
| | | UUUCUGCUGAAAUACAACGAAAACGGCACCAUCACAGACGCUGUGGACUGCGCUCUGGACCCCCUGUCCGAGACU |
| | | AAGUGCACCCUGAAGUCCUUCACCGUUGAAAAGGGCAUUUAUCAGACUUCCAACUUCCGCGUGCAGCCUACUGAG |
| | | UCCAUUGUGCGCUUCCCUAACAUCACUAACUUGUGUCCUUUCGGCGAAGUGUUCAACGCUACUCGCUUCGCCUCC |
| | | GUGUACGCCUGGAACCGCAAGAGAAUCUCCAAUUGCGUGGCCGACUACUCUGUUCUCUACAACUCUGCCUCCUUC |
| | | UCCACCUUUAAGUGCUACGGCGUGUCCCCCACCAAACUGAACGACCUGUGCUUCACCAACGUGUACGCCGACUCC |
| | | UUUGUGAUCAGAGGCGACGAGGUGCGCCAGAUCGCCCCCGGCCAGACUGGCAAGAUCGCCGAUUACAAUUACAA |
| | | GCUGCCUGACGACUUUACCGGCUGCGUGAUUGCCUGGAACUCCAACAACCUGGACUCCAAAGUGGGGGGCAACU |
| | | ACAACUACCUGUACCGCCUGUUUAGAAAAUCCAACCUGAAGCCCUUCGAGCGGGACAUUUCUACUGAGAUCUACC |
| | | AGGCCGGCUCCACCCCCUGUAACGGCGUGGAAGGCUUCAACUGCUACUUUCCCCUGCAGUCCUACGGCUUCCAG |
| | | CCUACCAACGGCGUGGGCUACCAGCCCUACCGCGUGGUGGUGUUAUCCUUCGAGCUGCUGCACGCCCCUGCCAC |
| | | CGUGUGCGGACCCAAAAAGUCUACUAACCUGGUUAAGAAUAAGUGUGUUAAUUUCAAUUUUAACGGCUUAACCGG |
| | | AACCGGAGUGCUGACCGAGUCCAAUAAAAAGUUCCUGCCCUUUCAGCAGUUUGGCCGCGACAUCGCCGAUACUAC |
| | | CGACGCCGUGCGCGAUCCCCAGACCCUGGAGAUCCUGGACAUUACCCCCUGCUCUUUCGGCGGAGUGUCCGUGA |
| | | UCACCCCCGGCACCAAUACCUCCAAUCAGGUGGCCGUUCUGUACCAGGACGUGAAUUGCACCGAAGUGCCCGUG |
| | | GCCAUUCACGCCGACCAGUUAACUCCUACUUGGAGAGUGUACUCCACCGGCUCUAACGUGUUUCAGACCCGCGCC |
| | | GGCUGUCUGAUCGGCGCCGAGCACGUGAACAACUCCUACGAGUGCGACAUUCCCAUUGGCGCCGGCAUCUGCGC |
| | | CUCCUACCAAACCCAGACUAACUCUCCCAGACGCGCCCGCUCUGUGGCCUCUCAGUCCAUCAUCGCUUACACUAU |
| | | GUCCCUGGGCGCCGAGAACUCCGUGGCCUAUUCCAACAACUCCAUCGCCAUCCCCACUAACUUCACCAUCUCUGU |
| | | GACCACCGAAAUCCUCCCCGUGUCUAUGACUAAGACUUCCGUGGACUGUACCAUGUACAUUUGCGGCGACUCCAC |
| | | CGAGUGUUCCAAUCUGCUGCUGCAGUACGGCUCCUUUUGCACCCAGCUGAACCGCGCCCUGACCGGCAUCGCUG |
| | | UUGAGCAGGACAAGAAUACCCAGGAGGUGUUUGCUCAGGUGAAGCAAAUCUAUAAGACCCCUCCCAUCAAGGACU |
| | | UUGGCGGCUUCAACUUUUCCCAGAUCCUGCCCGACCCUUCCAAGCCCUCCAAACGCUCUUUCAUCGAAGAUCUGC |
| | | UGUUCAAUAAAGUGACCCUGGCCGACGCCGGCUUCAUCAAGCAGUACGGAGACUGUCUGGGAGACAUUGCUGCU |
| | | CGCGACCUGAUUUGCGCCCAGAAGUUCAACGGCCUGACCGUGCUGCCCCCUCUGCUGACCGACGAGAUGAUCGC |
| | | UCAGUACACCUCCGCCCUGCUGGCUGGCACUAUCACCUCCGGCUGGACUUUCGGCGCUGGCGCCGCCCUGCAGA |
| | | UUCCCUUCGCCAUGCAGAUGGCUUAUCGCUUCAACGGAAUCGGAGUGACACAGAACGUGCUGUACGAGAAUCAGA |
| | | AACUGAUCGCUAACCAGUUUAAUUCCGCCAUUGGCAAGAUCCAGGACUCCCUGUCCUCUACUGCUUCCGCCCUGG |
| | | GCAAGCUGCAGGACGUGGUGAACCAGAACGCCCAGGCUCUGAACACCCUGGUGAAGCAACUGUCUUCUAACUUUG |
| | | GAGCCAUUUCUUCCGUGCUGAACGACAUCCUGUCCAGACUGGAUCCUCCUGAGGCCGAGGUGCAGAUCGACCGC |
| | | CUGAUCACUGGCCGCCUGCAGUCCCUGCAGACUUACGUUACCCAGCAAUUAAUCCGCGCUGCCGAGAUCCGCGC |
| | | CUCCGCCAACCUGGCCGCCACCAAGAUGUCCGAGUGCGUUCUGGGCCAGUCCAAACGCGUUGACUUCUGCGGCA |
| | | AGGGCUACCACCUGAUGUCCUUCCCCCAGUCCGCUCCCCACGGAGUGGUGUUCCUGCACGUGACCUACGUGCCU |
| | | GCCCAGGAGAAGAACUUCACCACCGCCCCCGCCAUUUGCCACGACGGAAAGGCCCACUUCCCCCGCGAGGGAGU |
| | | GUUCGUUUCUAACGGCACUCACUGGUUCGUGACCCAGAGAAACUUCUACGAGCCCCAGAUUAUUACCACCGACAA |
| | | UACCUUCGUUUCUGGCAACUGCGACGUGGUUAUCGGCAUCGUGAACAACACCGUGUACGACCCCCUGCAACCCGA |
| | | GCUGGAUUCUUUCAAGGAGGAGCUGGAUAAGUACUUUAAAAACCAUACCUCCCCCGACGUGGACCUGGGCGACAU |
| | | CUCUGGCAUCAACGCCUCCGUGGUGAAUAUCCAGAAGGAGAUCGAUCGCCUGAACGAGGUGGCCAAGAACUUAAA |
| | | CGAAUCUCUGAUCGACCUGCAGGAGCUGGGCAAGUACGAGCAGUAUAUUAAGUGGCCCUGGUAUAUCUGGUUAG |
| | | GCUUUAUCGCCGGCCUGAUCGCCAUUGUUAUGGUGACCAUCAUGCUGUGCUGCAUGACCUCCUGCUGUUCCUGU |
| | | UUAAAAGGCUGCUGCUCCUGCGGAUCCUGCUGUAAAUUCGACGAAGACGACUCCGAGCCCGUGCUGAAAGGAGU |
| | | UAAGUUACACUAUACCUAAAUGAGACCAUA |
| 158 | 2A5681 | AUAGGUCUCACACACUAGGGCGCGCCAGUCCUCAAGCUGGGCCUGACACAACUGUGUUCACUAGCAACUACUCCC |
| | | AGACACCAUGGUACUCCCGACUCCUGAGGUACUCCCUGCCGUUACUGCCCUGUGGGGCAAGGUGAUACUCCCUG |
| | | AAGUUGGUGGUGAGGCCCUGGGCAGGCUACUCCCGGUCUACCCUUGGACCCAGAGGUCGACUCGGACCGGCCAC |
| | | CAUGUUCGUGUUCCUGGUGCUGCUGCCCCUGGUGUCCUCCCAGUGCGUCAACCUGACCACCCGGACCCAACUGC |
| | | CCCCUGCCUAUACCAAUUCCUUCACCCGGGGCGUCUACUACCCCGACAAGGUGUUCCGGUCCUCUGUGCUCCAC |
| | | UCCACCCAGGACCUCUUCCUCCCCUUUUUCUCCAACGUGACCUGGUUCCACGCCAUCCACGUGUCUGGCACAAAC |
| | | GGAACCAAGCGCUUCGACAACCCUGUGCUCCCCUUCAACGACGGCGUGUACUUCGCCUCCACCGAGAAGUCCAAC |
| | | AUCAUCCGCGGCUGGAUCUUCGGCACCACCCUCGACUCUAAGACCCAGUCCCUGCUGAUUGUGAAUAACGCCACC |
| | | AACGUGGUGAUCAAGGUGUGCGAAUUUCAGUUCUGCAACGACCCCUUCCUGGGCGUUUACUACCAUAAGAACAAC |
| | | AAGUCCUGGAUGGAGUCCGAGUUCCGGGUGUAUUCCUCUGCCAAUAACUGCACCUUUGAGUACGUGUCCCAGCC |
| | | CUUCCUGAUGGACCUGGAGGGCAAGCAGGGCAAUUUUAAAAACCUGCGCGAGUUCGUGUUCAAGAACAUCGACG |
| | | GCUACUUCAAGAUCUAUUCCAAGCACACCCCUAUCAACCUGGUGCGCGACCUGCCCCAGGGCUUUUCCGCUCUGG |
| | | AGCCCCUGGUUGACCUGCCCAUCGGCAUCAACAUCACCCGCUUCCAGACUUUACUGGCCCUGCACCGCUCCUACC |
| | | UGACCCCCGGCGAUUCCUCUUCUGGCUGGACAGCCGGAGCCGCUGCCUACUACGUGGGAUACCUGCAGCCCCGC |
| | | ACCUUCCUGCUGAAGUACAACGAGAACGGAACCAUUACAGACGCUGUCGACUGUGCUCUGGAUCCCCUGUCCGAA |
| | | ACAAAGUGCACCCUGAAGUCCUUCACCGUGGAGAAGGGCAUCUACCAGACCUCCAACUUCCGCGUGCAGCCCACC |
| | | GAGUCCAUCGUGAGAUUCCCCAACAUCACUAACCUGUGCCCCUUCGGCGAGGUGUUCAACGCCACCCGCUUCGC |
| | | CUCCGUGUACGCCUGGAACCGCAAGCGCAUCUCCAACUGCGUUGCCGACUACUCUGUGCUCUACAACUCCGCCU |
| | | CCUUCUCCACCUUCAAGUGCUACGGCGUGUCCCCCACCAAGCUGAACGACCUGUGCUUCACCAACGUGUACGCCG |
| | | ACUCCUUCGUGAUCCGCGGCGACGAGGUGCGCCAGAUCGCCCCCGGCCAGACCGGAAAGAUCGCCGAUUACAAU |
| | | UACAAGCUGCCCGACGACUUCACCGGCUGCGUGAUCGCCUGGAACUCCAACAAUCUGGACUCCAAGGUUGGCGG |
| | | CAACUACAACUACCUGUACCGCCUGUUCCGCAAGUCCAACCUGAAGCCCUUCGAGCGGGACAUUUCCACAGAGAU |
| | | UUACCAGGCUGGCUCCACCCCCUGUAACGGAGUGGAGGGCUUCAACUGCUACUUCCCCCUGCAGUCCUACGGCU |
| | | UCCAGCCCACCAACGGCGUGGGCUACCAGCCCUACCGCGUGGUGGUGCUGUCCUUCGAGCUGCUGCACGCCCCU |
| | | GCCACCGUGUGCGGCCCCAAGAAGUCCACCAACCUGGUGAAGAACAAGUGCGUGAACUUCAACUUCAACGGACUG |
| | | ACAGGCACCGGCGUGCUGACCGAGUCCAACAAGAAGUUCCUGCCCUUCCAGCAGUUCGGCCGGGACAUUGCCGA |
| | | CACCACCGACGCCGUGCGCGACCCCCAGACCCUGGAGAUCCUGGACAUCACCCCCUGCUCCUUCGGCGGAGUGU |
| | | CCGUGAUCACCCCCGGCACCAACACCUCCAACCAGGUUGCCGUGCUGUACCAGGACGUUAACUGCACCGAGGUG |
| | | CCCGUGGCCAUCCACGCCGACCAGCUGACCCCCACCUGGCGCGUGUACUCCACCGGCUCCAACGUGUUCCAGAC |
| | | CCGCGCCGGCUGCCUGAUCGGCGCCGAGCACGUGAACAACUCCUACGAGUGCGACAUCCCCAUUGGCGCCGGCA |
| | | UCUGCGCCUCCUACCAGACCCAGACCAACUCCCCUCGCCGCGCCCGGUCCGUCGCCUCCCAGUCCAUCAUCGCC |
| | | UACACCAUGUCCCUGGGCGCCGAGAACUCCGUGGCCUACUCCAACAACUCCAUUGCCAUCCCCACCAACUUCACC |
| | | AUCUCCGUGACCACCGAAAUCCUCCCCGUGUCCAUGACCAAGACCUCCGUGGACUGCACCAUGUACAUCUGCGGC |
| | | GACUCCACCGAGUGCUCCAACCUGCUGCUGCAGUACGGCUCCUUCUGCACCCAGCUGAAUCGCGCCCUGACCGG |
| | | CAUCGCCGUGGAGCAGGACAAGAACACCCAAGAGGUGUUCGCCCAGGUGAAGCAGAUCUACAAGACACCUCCCAU |
| | | CAAGGACUUCGGCGGCUUCAACUUCUCCCAGAUCCUGCCCGACCCCUCCAAGCCCUCCAAGCGCUCCUUCAUCGA |
| | | GGACCUGCUGUUCAACAAGGUGACCCUGGCCGACGCCGGCUUCAUCAAGCAGUACGGCGACUGUCUGGGAGACA |
| | | UUGCCGCCCGCGACCUGAUCUGCGCCCAGAAGUUCAACGGCUUAACCGUGCUGCCCCCUCUGCUGACCGACGAG |
| | | AUGAUCGCCCAGUACACAUCCGCUCUGCUGGCCGGCACCAUCACCUCCGGCUGGACCUUUGGCGCUGGCGCUGC |
| | | CCUGCAGAUCCCCUUCGCUAUGCAGAUGGCCUACCGCUUCAACGGCAUCGGCGUGACCCAGAACGUGCUGUACG |
| | | AGAACCAGAAGCUGAUCGCCAACCAGUUCAACUCCGCCAUCGGCAAGAUCCAGGACUCCCUGUCCUCCACCGCCU |
| | | CCGCCCUGGGCAAGCUGCAGGACGUGGUGAACCAGAACGCCCAGGCCCUGAACACCCUGGUGAAGCAGCUGUCC |
| | | UCUAACUUCGGCGCCAUCUCCUCCGUGCUGAACGACAUUCUGUCCCGCCUGGAUCCUCCUGAGGCCGAGGUGCA |
| | | AAUCGAUCGCCUCAUCACCGGCCGCCUGCAGUCCCUGCAGACCUACGUGACACAGCAGCUGAUCCGCGCCGCCG |
| | | AGAUCCGCGCCUCCGCCAACCUGGCCGCCACCAAGAUGUCUGAGUGCGUGCUGGGCCAGUCCAAACGCGUUGAC |
| | | UUCUGCGGCAAGGGCUACCACCUGAUGUCCUUCCCCCAGUCCGCCCCCCACGGCGUGGUGUUCCUGCACGUGAC |
| | | CUACGUGCCCGCCCAGGAGAAGAACUUCACUACCGCCCCCGCCAUCUGCCACGACGGCAAGGCCCACUUCCCCCG |
| | | CGAGGGCGUGUUCGUGUCCAACGGCACCCACUGGUUCGUGACCCAGCGCAACUUCUACGAGCCCCAGAUCAUCA |
| | | CCACCGACAACACCUUCGUGUCCGGCAACUGCGACGUGGUGAUUGGCAUCGUGAACAACACCGUGUACGACCCCC |
| | | UGCAGCCCGAGCUGGACUCCUUCAAGGAGGAGCUGGACAAGUACUUUAAGAACCACACCUCCCCCGACGUGGACC |
| | | UGGGCGACAUCUCCGGCAUCAACGCCUCCGUGGUGAACAUCCAGAAGGAGAUCGACCGCCUGAACGAGGUGGCC |
| | | AAGAACCUGAACGAGUCCCUGAUCGACCUGCAGGAGCUGGGCAAGUACGAACAGUACAUCAAGUGGCCCUGGUAC |
| | | AUCUGGCUGGGCUUCAUCGCCGGCCUGAUCGCCAUCGUGAUGGUGACCAUCAUGCUGUGCUGCAUGACCUCUUG |
| | | CUGUUCCUGUCUGAAGGGCUGCUGCUCCUGCGGCUCCUGCUGCAAGUUCGACGAGGACGACUCCGAGCCCGUGC |
| | | UGAAGGGCGUGAAGCUGCACUACACCUAAAUGAGACCAUA |
| 159 | 2A6681 | AUAGGUCUCACACACUAGGGCGCGCCAGUCCUCAAGCUGGGCCUGACACAACUGUGUUCACUAGCAACUACUCCC |
| | | AGACACCAUGGUACUCCCGACUCCUGAGGUACUCCCUGCCGUUACUGCCCUGUGGGGCAAGGUGAUACUCCCUG |
| | | AAGUUGGUGGUGAGGCCCUGGGCAGGCUACUCCCGGUCUACCCUUGGACCCAGAGGUCGACUCGGACCGGCCAC |
| | | CAUGUUCGUGUUCCUGGUGCUGCUCCCCCUGGUGAGCUCCCAGUGCGUCAACCUGACCACACGGACCCAGCUGC |
| | | CCCCCGCCUAUACCAACAGCUUCACCCGCGGCGUCUACUACCCCGACAAGGUGUUCCGGAGCAGCGUGCUGCAC |
| | | AGCACCCAGGACCUCUUCCUCCCCUUUUUUAGCAACGUGACCUGGUUCCACGCCAUCCACGUGAGUGGCACCAAC |
| | | GGCACCAAGCGGUUCGACAACCCUGUGCUCCCCUUCAACGACGGCGUGUACUUCGCUAGCACAGAAAAAAGUAAC |
| | | AUCAUUCGGGGCUGGAUCUUCGGCACCACACUGGAUAGCAAAACCCAGAGCCUGCUGAUUGUGAAUAACGCCACC |
| | | AACGUGGUGAUCAAGGUGUGCGAGUUCCAGUUCUGCAACGACCCCUUCCUGGGCGUGUACUACCACAAGAACAAC |
| | | AAGAGCUGGAUGGAGAGCGAGUUCCGCGUGUAUAGCUCUGCCAACAACUGCACCUUCGAGUACGUGAGCCAGCC |
| | | CUUCCUGAUGGACCUGGAGGGCAAGCAGGGCAAUUUCAAAAACCUGCGGGAGUUCGUGUUCAAGAACAUCGACG |
| | | GCUACUUCAAGAUCUACAGCAAGCACACCCCCAUCAACCUGGUGCGGGACCUGCCCCAGGGCUUUAGCGCUCUG |
| | | GAGCCCCUGGUGGACCUGCCCAUCGGCAUCAACAUCACCCGCUUCCAAACCCUGUUGGCCCUGCACCGGAGCUA |
| | | CCUGACCCCUGGCGAUAGCAGUAGUGGCUGGACAGCCGGAGCCGCCGCCUACUACGUGGGAUACCUGCAGCCCC |
| | | GGACCUUCCUGCUGAAGUACAACGAGAACGGCACAAUCACAGACGCCGUGGACUGCGCCCUGGACCCCCUGAGC |
| | | GAAACAAAGUGCACCCUCAAAAGUUUCACCGUGGAGAAGGGCAUCUACCAGACCAGCAACUUCCGGGUGCAGCCC |
| | | ACCGAGAGCAUCGUGCGGUUCCCCAACAUCACCAACCUGUGCCCCUUCGGCGAGGUGUUCAACGCCACCCGGUU |
| | | CGCCAGCGUGUACGCCUGGAACCGGAAGCGGAUCAGCAACUGCGUGGCCGACUACAGCGUGCUGUAUAACAGUG |
| | | CCAGCUUCAGCACCUUCAAGUGCUACGGCGUGAGCCCCACCAAGCUGAACGACCUGUGCUUCACCAACGUGUACG |
| | | CCGACAGCUUCGUGAUCCGGGGCGACGAGGUGCGGCAGAUCGCCCCCGGCCAGACCGGCAAGAUCGCCGAUUAC |
| | | AAUUACAAGCUGCCCGACGAUUUCACCGGCUGCGUGAUCGCCUGGAACAGCAACAACCUGGACAGCAAGGUGGGC |
| | | GGCAACUACAACUACCUGUACCGGCUGUUCCGCAAAAGUAACCUGAAGCCCUUCGAGCGGGACAUCAGCACCGAG |
| | | AUCUACCAGGCCGGCAGUACCCCUUGCAACGGCGUCGAGGGCUUCAACUGCUACUUCCCCCUGCAGAGCUACGG |
| | | CUUCCAGCCCACCAACGGCGUGGGCUACCAGCCCUACCGGGUGGUGGUUCUGAGCUUCGAGCUGCUGCACGCCC |
| | | CUGCCACCGUGUGCGGCCCCAAGAAAAGCACCAACCUGGUGAAGAACAAGUGCGUGAAUUUCAAUUUUAACGGAC |
| | | UCACCGGAACCGGCGUGCUGACCGAGAGCAACAAGAAGUUCCUGCCCUUCCAGCAAUUCGGCCGGGAUAUUGCC |
| | | GACACCACUGACGCCGUGCGGGACCCCCAGACCCUGGAGAUCCUGGACAUCACACCCUGUAGCUUCGGCGGGGU |
| | | GAGCGUGAUCACCCCCGGCACCAACACCAGCAACCAGGUGGCCGUGCUGUACCAGGACGUGAACUGCACCGAGG |
| | | UGCCCGUGGCCAUCCACGCCGACCAGCUGACCCCCACCUGGGGGGUGUACAGCACCGGCAGCAACGUGUUCCAG |
| | | ACCCGGGCCGGCUGCCUGAUCGGCGCCGAGCACGUGAAUAACAGCUACGAGUGCGACAUCCCCAUCGGAGCCGG |
| | | AAUCUGCGCCAGCUACCAGACCCAGACCAACAGCCCCCGGAGAGCCCGGAGCGUGGCCAGCCAGAGCAUUAUUGC |
| | | CUACACCAUGAGCCUGGGCGCCGAGAACAGCGUGGCCUACAGCAACAAUAGUAUUGCCAUCCCCACCAACUUCAC |
| | | CAUCAGCGUGACCACCGAAAUCCUCCCCGUGAGCAUGACCAAGACCAGCGUGGACUGCACCAUGUACAUCUGCGG |
| | | CGACAGCACCGAGUGCAGCAACCUGCUCCUGCAGUACGGCAGCUUCUGCACCCAGCUGAACCGGGCCCUGACCG |
| | | GAAUUGCCGUGGAGCAGGACAAGAACACCCAGGAGGUGUUCGCCCAGGUGAAGCAGAUCUACAAGACACCUCCCA |
| | | UCAAGGACUUCGGCGGCUUCAACUUCAGCCAGAUCCUGCCCGACCCCAGCAAGCCCAGCAAGCGGAGCUUCAUCG |
| | | AGGACCUGUUGUUCAACAAGGUGACCCUGGCCGACGCCGGCUUCAUCAAGCAGUACGGCGACUGUCUGGGAGAU |
| | | AUUGCCGCACGGGACCUGAUCUGCGCCCAGAAGUUCAACGGCCUGACCGUGCUGCCCCCACUGCUGACCGACGA |
| | | GAUGAUCGCCCAGUACACAAGCGCUCUGCUCGCCGGCACCAUCACCAGCGGCUGGACCUUCGGCGCCGGCGCUG |
| | | CCCUGCAGAUCCCCUUCGCCAUGCAGAUGGCCUACCGGUUCAACGGCAUCGGCGUGACCCAGAACGUGCUGUAC |
| | | GAGAACCAGAAGCUGAUCGCCAACCAGUUCAACAGCGCCAUCGGCAAGAUCCAGGACAGCCUGAGCUCCACCGCC |
| | | AGCGCCCUGGGCAAGCUGCAGGACGUGGUGAACCAGAACGCCCAGGCCCUGAACACCCUGGUGAAGCAGCUGAG |
| | | CAGCAACUUCGGCGCCAUCAGCUCUGUGCUGAACGACAUCCUGAGCCGGCUGGAUCCUCCUGAGGCCGAGGUGC |
| | | AAAUCGAUCGGCUCAUCACCGGCCGGCUGCAGAGCCUGCAGACCUACGUGACACAGCAGCUGAUCCGGGCCGCC |
| | | GAGAUCCGGGCCAGCGCCAACCUGGCCGCUACCAAGAUGAGCGAGUGCGUGCUGGGCCAGAGCAAGCGGGUGGA |
| | | CUUCUGCGGCAAGGGCUACCACCUGAUGAGCUUCCCCCAGAGCGCCCCCCACGGCGUGGUGUUCCUGCACGUGA |
| | | CCUACGUGCCCGCCCAGGAGAAGAACUUCACCACCGCCCCCGCCAUCUGCCACGACGGCAAGGCCCACUUCCCCC |
| | | GGGAGGGCGUGUUCGUGAGCAACGGCACCCACUGGUUCGUGACCCAGCGGAACUUCUACGAGCCCCAGAUCAUA |
| | | ACCACAGACAACACCUUCGUGAGCGGCAACUGCGACGUGGUCAUCGGCAUCGUGAACAACACCGUGUACGACCCC |
| | | CUGCAGCCCGAGCUGGACAGCUUCAAGGAGGAACUGGACAAGUACUUCAAGAACCACACCAGCCCCGACGUGGAC |
| | | CUGGGCGACAUCAGCGGCAUCAACGCCAGCGUGGUGAACAUCCAGAAGGAGAUCGACCGGCUGAACGAGGUGGC |
| | | CAAGAACCUGAACGAGAGCCUGAUCGACCUGCAGGAGCUGGGCAAGUACGAGCAGUACAUCAAGUGGCCCUGGUA |
| | | CAUCUGGCUGGGCUUUAUUGCCGGCCUGAUCGCCAUCGUGAUGGUGACCAUCAUGCUGUGCUGUAUGACAAGCU |
| | | GUUGCAGUUGCCUGAAGGGCUGCUGCAGCUGCGGCAGCUGCUGCAAGUUCGACGAGGACGACAGCGAGCCCGU |
| | | GCUGAAGGGCGUGAAGCUGCACUACACCUAAAUGAGACCAUA |
| 160 | P51 | AUGUACCGGAUGCAGUUGUUGUCCUGUAUAGCUCUUUCCCUUGCAUUGGUCACUAAUUCUGUGAACCUCACCACU |
| | | CGCACACAGCUACCUCCCGCGUACACUAAUUCAUUUACCAGGGGCGUCUAUUAUCCUGAUAAGGUGUUCCGGAGU |
| | | UCAGUGUUGCAUAGCACUCAAGACCUGUUCCUGCCCUUCUUCUCCAAUGUCACUUGGUUUCAUGCGAUACAUGUG |
| | | UCUGGUACCAACGGAACGAAGAGAUUUGAUAACCCCGUACUGCCAUUCAAUGAUGGCGUAUACUUUGCUUCGACU |
| | | GAAAAAUCCAACAUCAUCAGGGGCUGGAUUUUUGGUACAACGCUUGAUUCCAAGACCCAGUCCCUCCUUAUUGUG |
| | | AACAAUGCGACCAACGUCGUGAUAAAGGUCUGUGAGUUUCAGUUCUGCAAUGACCCAUUCCUUGGAGUGUAUUAU |
| | | CACAAGAACAACAAAUCGUGGAUGGAGUCAGAGUUUAGGGUGUACAGCAGCGCUAACAACUGUACAUUUGAGUAU |
| | | GUGAGUCAGCCGUUUCUGAUGGAUCUUGAGGGCAAACAGGGCAAUUUUAAGAAUCUCAGAGAAUUUGUGUUCAAG |
| | | AACAUUGAUGGUUACUUUAAGAUCUAUAGCAAACAUACGCCAAUCAACUUGGUUCGUGAUCUGCCACAGGGAUUU |
| | | AGCGCACUGGAACCUCUCGUUGACUUGCCCAUAGGUAUUAACAUCACCAGGUUCCAGACGCUCUUGGCAUUACAC |
| | | CGUAGUUAUCUGACCCCCGGGGACUCCAGUUCCGGAUGGACUGCAGGAGCCGCUGCCUACUAUGUGGGUUACCU |
| | | CCAGCCCAGGACCUUUCUUUUGAAAUAUAACGAGAACGGCACAAUCACUGAUGCUGUGGACUGCGCAUUGGAUCC |
| | | UUUGUCAGAGACUAAGUGCACUCUGAAGUCAUUCACAGUCGAGAAAGGCAUUUACCAGACGUCUAACUUCAGGGU |
| | | UCAGCCUACUGAGUCCAUCGUGAGAUUCCCAAACAUCACAAAUCUUUGUCCCUUCGGUGAGGUAUUCAAUGCGAC |
| | | ACGAUUUGCCUCAGUGUACGCGUGGAAUCGGAAGAGGAUCUCCAAUUGCGUGGCCGACUACUCCGUCUUAUACAA |
| | | CUCAGCUAGCUUUUCAACAUUCAAGUGCUAUGGCGUGAGCCCUACCAAGCUCAAUGACCUGUGCUUCACUAAUGU |
| | | GUAUGCCGACUCUUUUGUCAUUCGCGGCGACGAGGUCCGACAAAUCGCACCGGGCCAAACCGGUAAAAUUGCCG |
| | | ACUACAACUACAAGCUGCCUGACGACUUCACCGGCUGCGUAAUCGCCUGGAACAGCAAUAACCUGGAUAGCAAAG |
| | | UGGGCGGAAACUACAACUACCUGUACCGGCUCUUUAGAAAGUCCAACCUGAAACCAUUCGAGCGCGAUAUCUCGA |
| | | CCGAAAUCUACCAGGGGGGCAGCACCCCCUGUAAUGGUGUAGAAGGGUUCAAUUGUUACUUUCCACUCCAGAGUU |
| | | AUGGGUUCCAGCCGACCAAUGGCGUCGGUUAUCAACCAUAUCGCGUUGUGGUGUUGUCCUUUGAGCUGCUACAC |
| | | GCCCCAGCUACAGUGUGCGGGCCAAAGAAAAGCACAAAUCUUGUCAAGAACAAAUGCGUUAACUUUAAUUUUAAUG |
| | | GACUCACAGGUACAGGAGUCCUGACCGAAUCUAAUAAGAAGUUCCUGCCCUUUCAACAGUUCGGACGAGACAUUG |
| | | CCGACACCACCGAUGCCGUUCGGGACCCACAGACCUUAGAAAUUCUGGAUAUUACUCCAUGUAGUUUUGGGGGAG |
| | | UGUCUGUCAUCACCCCUGGCACUAAUACAUCUAACCAGGUUGCAGUCCUCUACCAGGAUGUGAACUGUACCGAAG |
| | | UGCCGGUCGCUAUUCACGCAGACCAGCUCACUCCUACCUGGGGGGUGUACUCCACAGGCUCUAACGUGUUUCAG |
| | | ACACGUGCAGGGUGCCUAAUCGGCGCAGAGCAUGUAAAUAACUCCUAUGAGUGUGAUAUCCCCAUCGGAGCCGG |
| | | GAUCUGCGCUUCCUACCAGACACAAACGAAUAGUCCCGGAUCUGCCUCAAGCGUGGCAUCUCAAUCCAUUAUAGC |
| | | AUAUACGAUGUCCCUUGGAGCUGAAAACAGCGUUGCGUAUUCAAACAAUAGUAUCGCUAUUCCAACCAAUUUUACA |
| | | AUUAGCGUGACCACAGAAAUACUCCCUGUGAGCAUGACCAAGACCAGUGUAGACUGUACUAUGUACAUCUGCGGC |
| | | GACAGUACUGAGUGUAGCAAUCUGCUGCUACAGUAUGGGUCCUUCUGUACUCAGCUUAAUCGGGCUCUCACCGG |
| | | AAUCGCUGUAGAGCAAGAUAAAAACACACAAGAAGUGUUUGCUCAAGUGAAGCAGAUCUAUAAGACACCUCCCAUC |
| | | AAGGAUUUCGGUGGGUUCAACUUUAGCCAGAUUCUGCCCGAUCCGUCUAAACCGUCCAAGCGAAGUUUCAUCGAA |
| | | GACCUGCUUUUCAAUAAGGUCACGCUGGCAGAUGCUGGAUUUAUCAAACAGUACGGCGACUGUCUGGGCGAUAU |
| | | CGCCGCAAGAGACUUGAUAUGCGCCCAAAAGUUUAAUGGGUUAACCGUCCUUCCACCGCUCCUGACAGACGAGAU |
| | | GAUCGCCCAGUAUACAAGUGCCUUAUUAGCUGGGACCAUUACUAGUGGAUGGACAUUUGGCGCCGGGGCUGCUC |
| | | UACAGAUACCCUUCGCCAUGCAGAUGGCUUACCGCUUCAACGGAAUCGGAGUUACCCAGAACGUACUGUACGAAA |
| | | AUCAGAAACUCAUAGCUAAUCAAUUUAACUCUGCCAUCGGGAAGAUUCAGGAUUCCCUGUCGUCUACAGCGUCCG |
| | | CCUUGGGGAAACUGCAAGAUGUAGUGAACCAGAACGCCCAGGCCUUAAAUACUCUGGUCAAGCAGUUAUCUUCAA |
| | | AUUUCGGAGCAAUUAGCUCUGUGUUGAACGAUAUUCUUUCCAGGCUGGACCCUCCAGAAGCCGAAGUGCAAAUAG |
| | | ACCGGCUCAUCACGGGGCGCUUGCAAAGCCUGCAAACCUAUGUCACCCAGCAACUGAUUCGAGCAGCCGAGAUCC |
| | | GGGCCAGUGCUAAUCUGGCCGCCACAAAAAUGAGCGAGUGCGUCCUCGGGCAGAGCAAACGCGUAGACUUCUGC |
| | | GGUAAAGGCUAUCACCUGAUGAGCUUCCCUCAGAGCGCACCCCACGGGGUGGUCUUCCUCCACGUUACCUACGU |
| | | CCCUGCGCAGGAGAAGAACUUCACUACGGCCCCUGCAAUUUGCCACGAUGGCAAGGCCCACUUUCCCAGGGAGG |
| | | GGGUCUUCGUUUCCAACGGGACUCAUUGGUUCGUGACUCAGAGAAAUUUUUAUGAACCUCAGAUCAUUACCACUG |
| | | AUAAUACAUUCGUGUCUGGCAACUGUGAUGUGGUUAUUGGGAUAGUUAAUAAUACGGUAUACGACCCACUCCAGC |
| | | CCGAGCUGGACUCCUUCAAAGAGGAGCUGGACAAGUACUUUAAAAAUCACACCUCACCUGAUGUGGACCUAGGUG |
| | | ACAUAUCUGGCAUAAAUGCUAGCGUGGUUAACAUUCAGAAGGAAAUCGACAGACUCAACGAGGUGGCCAAAAAUC |
| | | UGAACGAGAGUCUGAUCGACCUGCAGGAGUUGGGAAAAUAUGAACAGGGGUAUAUCCCUGAAGCCCCCAGGGAC |
| | | GGCCAGGCUUACGUCAGAAAGGAUGGAGAGUGGGUGCUCUUGAGCACCUUCCUGUAA |
| 161 | P53 | AUGGGAGUCAAAGUUCUGUUUGCCCUGAUCUGCAUCGCUGUGGCCGAGGCCAAGGUUAAUCUUACAACCAGAACU |
| | | CAAUUACCCCCUGCAUACACUAAUUCUUUCACACGUGGUGUUUAUUACCCUGACAAAGUUUUCAGAUCCUCAGUU |
| | | UUACAUUCAACUCAGGACUUGUUCUUACCUUUCUUUUCCAAUGUUACUUGGUUCCAUGCUAUACAUGUCUCUGGG |
| | | ACCAAUGGUACUAAGAGGUUUGAUAACCCUGUCCUACCAUUUAAUGAUGGUGUUUAUUUUGCUUCCACUGAGAAG |
| | | UCUAACAUAAUAAGAGGCUGGAUUUUUGGUACUACUUUAGAUUCGAAGACCCAGUCCCUACUUAUUGUUAAUAAC |
| | | GCUACUAAUGUUGUUAUUAAAGUCUGUGAAUUUCAAUUUUGUAAUGAUCCAUUUUUGGGUGUUUAUUACCACAAA |
| | | AACAACAAAAGUUGGAUGGAAAGUGAGUUCAGAGUUUAUUCUAGUGCGAAUAAUUGCACUUUUGAAUAUGUCUCU |
| | | CAGCCUUUUCUUAUGGACCUUGAAGGAAAACAGGGUAAUUUCAAAAAUCUUAGGGAAUUUGUGUUUAAGAAUAUU |
| | | GAUGGUUAUUUUAAAAUAUAUUCUAAGCACACGCCUAUUAAUUUAGUGCGUGAUCUCCCUCAGGGUUUUUCGGCU |
| | | UUAGAACCAUUGGUAGAUUUGCCAAUAGGUAUUAACAUCACUAGGUUUCAAACUUUACUUGCUUUACAUAGAAGUU |
| | | AUUUGACUCCUGGUGAUUCUUCUUCAGGUUGGACAGCUGGUGCUGCAGCUUAUUAUGUGGGUUAUCUUCAACCU |
| | | AGGACUUUUCUAUUAAAAUAUAAUGAAAAUGGAACCAUUACAGAUGCUGUAGACUGUGCACUUGACCCUCUCUCAG |
| | | AAACAAAGUGUACGUUGAAAUCCUUCACUGUAGAAAAAGGAAUCUAUCAAACUUCUAACUUUAGAGUCCAACCAAC |
| | | AGAAUCUAUUGUUAGAUUUCCUAAUAUUACAAACUUGUGCCCUUUUGGUGAAGUUUUUAACGCCACCAGAUUUGC |
| | | AUCCGUGUAUGCUUGGAACAGGAAGAGAAUCAGCAACUGUGUUGCUGAUUAUUCUGUCCUAUAUAAUUCCGCAUC |
| | | AUUUUCCACUUUUAAGUGUUAUGGAGUGUCUCCUACUAAAUUAAAUGAUCUCUGCUUUACUAAUGUCUAUGCAGA |
| | | UUCAUUUGUAAUUAGAGGUGAUGAAGUCAGACAAAUCGCUCCAGGGCAAACUGGAAAGAUUGCUGAUUAUAAUUA |
| | | UAAAUUACCAGAUGAUUUUACAGGCUGCGUUAUAGCUUGGAAUUCUAACAAUCUUGAUUCUAAGGUUGGUGGUAA |
| | | UUAUAAUUACCUGUAUAGAUUGUUUAGGAAGUCUAAUCUCAAACCUUUUGAGAGAGAUAUUUCAACUGAAAUCUAU |
| | | CAGGCCGGUAGCACACCUUGUAAUGGUGUUGAAGGUUUUAAUUGUUACUUUCCUUUACAAUCAUAUGGUUUCCAA |
| | | CCCACUAAUGGUGUUGGUUACCAACCAUACAGAGUAGUAGUACUUUCUUUUGAACUUCUACAUGCACCAGCAACU |
| | | GUUUGUGGACCUAAAAAGUCUACUAAUUUGGUUAAAAACAAAUGUGUCAAUUUCAACUUCAAUGGUUUAACAGGCA |
| | | CAGGUGUUCUUACUGAGUCUAACAAAAAGUUUCUGCCUUUCCAACAAUUUGGCAGAGACAUUGCUGACACUACUG |
| | | AUGCUGUCCGUGAUCCACAGACACUUGAGAUUCUUGACAUUACACCAUGUUCUUUUGGUGGUGUCAGUGUUAUAA |
| | | CACCAGGAACAAAUACUUCUAACCAGGUUGCUGUUCUUUAUCAGGAUGUUAACUGCACAGAAGUCCCUGUUGCUA |
| | | UUCAUGCAGAUCAACUUACUCCUACUUGGCGUGUUUAUUCUACAGGUUCUAAUGUUUUUCAAACACGUGCAGGCU |
| | | GUUUAAUAGGGGCUGAACAUGUCAACAACUCAUAUGAGUGUGACAUACCCAUUGGUGCAGGUAUAUGCGCUAGUU |
| | | AUCAGACUCAGACUAAUUCUCCUGGCAGCGCCAGCAGUGUAGCUAGUCAAUCCAUCAUUGCCUACACUAUGUCAC |
| | | UUGGUGCAGAAAAUUCAGUUGCUUACUCUAAUAACUCUAUUGCCAUACCCACAAAUUUUACUAUUAGUGUUACCAC |
| | | AGAAAUUCUACCAGUGUCUAUGACCAAGACAUCAGUAGAUUGUACAAUGUACAUUUGUGGUGAUUCAACUGAAUG |
| | | CAGCAAUCUUUUGUUGCAAUAUGGCAGUUUUUGUACACAAUUAAACCGUGCUUUAACUGGAAUAGCUGUUGAACA |
| | | AGACAAAAACACCCAAGAAGUUUUUGCACAAGUCAAACAAAUUUACAAAACACCACCAAUUAAAGAUUUUGGUGGU |
| | | UUUAAUUUUUCACAAAUAUUACCAGAUCCAUCAAAACCAAGCAAGAGGUCAUUUAUUGAAGAUCUACUUUUCAACA |
| | | AAGUGACACUUGCAGAUGCUGGCUUCAUCAAACAAUAUGGUGAUUGCCUUGGUGAUAUUGCUGCUAGGGACCUCA |
| | | UUUGUGCACAAAAGUUUAACGGCCUUACUGUUUUGCCACCUUUGCUCACAGAUGAAAUGAUUGCUCAAUACACUU |
| | | CUGCACUGUUAGCGGGUACAAUCACUUCUGGUUGGACCUUUGGUGCAGGUGCUGCAUUACAAAUACCAUUUGCU |
| | | AUGCAAAUGGCUUAUAGGUUUAAUGGUAUUGGAGUUACACAGAAUGUUCUCUAUGAGAACCAAAAAUUGAUUGCC |
| | | AACCAAUUUAAUAGUGCUAUUGGCAAAAUUCAAGACUCACUUUCUUCCACAGCAAGUGCACUUGGAAAACUUCAAG |
| | | AUGUGGUCAACCAAAAUGCACAAGCUUUAAACACGCUUGUUAAACAACUUAGCUCCAAUUUUGGUGCAAUUUCAAG |
| | | UGUUUUAAAUGAUAUCCUUUCACGUCUUGACCCUCCCGAGGCUGAAGUGCAAAUUGAUAGGUUGAUCACAGGCAG |
| | | ACUUCAAAGUUUGCAGACAUAUGUGACUCAACAAUUAAUUAGAGCUGCAGAAAUCAGAGCUUCUGCUAAUCUUGCU |
| | | GCUACUAAAAUGUCAGAGUGUGUACUUGGACAAUCAAAAAGAGUUGAUUUUUGUGGAAAGGGCUAUCAUCUUAUG |
| | | UCCUUCCCUCAGUCAGCACCUCAUGGUGUAGUCUUCUUGCAUGUGACUUAUGUCCCUGCACAAGAAAAGAACUUC |
| | | ACAACUGCUCCUGCCAUUUGUCAUGAUGGAAAAGCACACUUUCCUCGUGAAGGUGUCUUUGUUUCAAAUGGCACA |
| | | CACUGGUUUGUAACACAAAGGAAUUUUUAUGAACCACAAAUCAUUACUACAGACAACACAUUUGUGUCUGGUAACU |
| | | GUGAUGUUGUAAUAGGAAUUGUCAACAACACAGUUUAUGAUCCUUUGCAACCUGAAUUAGACUCAUUCAAGGAGG |
| | | AGUUAGAUAAAUAUUUUAAGAAUCAUACAUCACCAGAUGUUGAUUUAGGUGACAUCUCUGGCAUUAAUGCUUCAGU |
| | | UGUAAACAUUCAAAAAGAAAUUGACCGCCUCAAUGAGGUUGCCAAGAAUUUAAAUGAAUCUCUCAUCGAUCUCCAA |
| | | GAACUUGGAAAGUAUGAGCAGGGGUAUAUCCCUGAAGCCCCCAGGGACGGCCAGGCUUACGUCAGAAAGGAUGG |
| | | AGAGUGGGUGCUCUUGAGCACCUUCCUG |
| 162 | P55 | AUGGGAGUCAAAGUUCUGUUUGCCCUGAUCUGCAUCGCUGUGGCCGAGGCCAAGGUUAAUCUUACAACCAGAACU |
| | | CAAUUACCCCCUGCAUACACUAAUUCUUUCACACGUGGUGUUUAUUACCCUGACAAAGUUUUCAGAUCCUCAGUU |
| | | UUACAUUCAACUCAGGAUUUGUUCUUACCUUUCUUUUCCAAUGUUACUUGGUUCCAUGCUAUACAUGUCUCUGGG |
| | | ACCAAUGGUACUAAGAGGUUUGAUAACCCUGUCCUACCAUUUAAUGAUGGUGUUUAUUUUGCUUCCACUGAGAAG |
| | | UCUAACAUAAUAAGAGGCUGGAUCUUUGGUACUACUUUAGAUUCGAAGACCCAGUCCCUACUUAUUGUUAAUAAC |
| | | GCUACUAAUGUUGUUAUUAAAGUCUGUGAAUUUCAAUUUUGUAAUGAUCCAUUUUUGGGUGUUUAUUACCACAAA |
| | | AACAACAAAAGUUGGAUGGAAAGUGAGUUCAGAGUUUAUUCUAGUGCGAAUAAUUGCACUUUUGAAUAUGUCUCU |
| | | CAGCCUUUUCUUAUGGACCUUGAAGGAAAACAGGGUAAUUUCAAAAAUCUUAGGGAAUUUGUGUUUAAGAAUAUU |
| | | GAUGGUUAUUUUAAAAUAUAUUCUAAGCACACGCCUAUUAAUUUAGUGCGUGAUCUCCCUCAGGGUUUUUCGGCU |
| | | UUAGAACCAUUGGUAGAUUUGCCAAUAGGUAUUAACAUCACUAGGUUUCAAACUUUACUUGCUUUACAUAGAAGUU |
| | | AUUUGACUCCUGGUGAUUCUUCUUCAGGUUGGACAGCUGGUGCUGCAGCUUAUUAUGUGGGUUAUCUUCAACCU |
| | | AGGACUUUUCUAUUAAAAUAUAAUGAAAAUGGAACCAUUACAGAUGCUGUAGACUGUGCACUUGACCCUCUCUCAG |
| | | AAACAAAGUGUACGUUGAAAUCCUUCACUGUAGAAAAAGGAAUCUAUCAAACUUCUAACUUUAGAGUCCAACCAAC |
| | | AGAAUCUAUUGUUAGAUUUCCUAAUAUUACAAACUUGUGCCCUUUUGGUGAAGUUUUUAACGCCACCAGAUUUGC |
| | | AUCCGUGUAUGCUUGGAACAGGAAGAGAAUCAGCAACUGUGUUGCUGAUUAUUCUGUCCUAUAUAAUUCCGCAUC |
| | | AUUUUCCACUUUUAAGUGUUAUGGAGUGUCUCCUACUAAAUUAAAUGAUCUCUGCUUUACUAAUGUCUAUGCAGA |
| | | UUCAUUUGUAAUUAGAGGUGAUGAAGUCAGACAAAUCGCUCCAGGGCAAACUGGAAAGAUUGCUGAUUAUAAUUA |
| | | UAAAUUACCAGAUGAUUUUACAGGCUGCGUUAUAGCUUGGAAUUCUAACAAUCUUGAUUCUAAGGUUGGUGGUAA |
| | | UUAUAAUUACCUGUAUAGAUUGUUUAGGAAGUCUAAUCUCAAACCUUUUGAGAGAGAUAUUUCAACUGAAAUCUAU |
| | | CAGGCCGGUAGCACACCUUGUAAUGGUGUUGAAGGUUUUAAUUGUUACUUUCCUUUACAAUCAUAUGGUUUCCAA |
| | | CCCACUAAUGGUGUUGGUUACCAACCAUACAGAGUAGUAGUACUUUCUUUUGAACUUCUACAUGCACCAGCAACU |
| | | GUUUGUGGACCUAAAAAGUCUACUAAUUUGGUUAAAAACAAAUGUGUCAAUUUCAACUUCAAUGGUUUAACAGGCA |
| | | CAGGUGUUCUUACUGAGUCUAACAAAAAGUUUCUGCCUUUCCAACAAUUUGGCAGAGACAUUGCUGACACUACUG |
| | | AUGCUGUCCGUGAUCCACAGACACUUGAGAUUCUUGACAUUACACCAUGUUCUUUUGGUGGUGUCAGUGUUAUAA |
| | | CACCAGGAACAAAUACUUCUAACCAGGUUGCUGUUCUUUAUCAGGAUGUUAACUGCACAGAAGUCCCUGUUGCUA |
| | | UUCAUGCAGAUCAACUUACUCCUACUUGGCGUGUUUAUUCUACAGGUUCUAAUGUUUUUCAAACACGUGCAGGCU |
| | | GUUUAAUAGGGGCUGAACAUGUCAACAACUCAUAUGAGUGUGACAUACCCAUUGGUGCAGGUAUAUGCGCUAGUU |
| | | AUCAGACUCAGACUAAUUCUCCUGGCAGCGCCAGCAGUGUAGCUAGUCAAUCCAUCAUUGCCUACACUAUGUCAC |
| | | UUGGUGCAGAAAAUUCAGUUGCUUACUCUAAUAACUCUAUUGCCAUACCCACAAAUUUUACUAUUAGUGUUACCAC |
| | | AGAAAUUCUACCAGUGUCUAUGACCAAGACAUCAGUAGAUUGUACAAUGUACAUUUGUGGUGAUUCAACUGAAUG |
| | | CAGCAAUCUUUUGUUGCAAUAUGGCAGUUUUUGUACACAAUUAAACCGUGCUUUAACUGGGAUAGCUGUUGAACA |
| | | AGACAAAAACACCCAAGAAGUUUUUGCACAAGUCAAACAAAUUUACAAAACACCACCAAUUAAAGAUUUUGGUGGU |
| | | UUUAAUUUUUCACAAAUAUUACCAGAUCCAUCAAAACCAAGCAAGAGGUCAUUUAUUGAAGAUCUACUUUUCAACA |
| | | AAGUGACACUUGCAGAUGCUGGCUUCAUCAAACAAUAUGGUGAUUGCCUUGGUGAUAUUGCUGCUAGGGACCUCA |
| | | UUUGUGCACAAAAGUUUAACGGCCUUACUGUUUUGCCACCUUUGCUCACAGAUGAAAUGAUUGCUCAAUACACUU |
| | | CUGCACUGUUAGCGGGUACAAUCACUUCUGGUUGGACCUUUGGUGCAGGUGCUGCAUUACAAAUACCAUUUGCU |
| | | AUGCAAAUGGCUUAUAGGUUUAAUGGUAUUGGAGUUACACAGAAUGUUCUCUAUGAGAACCAAAAAUUGAUUGCC |
| | | AACCAAUUUAAUAGUGCCAUUGGCAAAAUUCAAGACUCACUUUCUUCCACAGCAAGUGCACUUGGAAAACUUCAAG |
| | | AUGUGGUCAACCAAAAUGCACAAGCUUUAAACACGCUUGUUAAACAACUUAGCUCCAAUUUUGGUGCAAUUUCAAG |
| | | UGUUUUAAAUGAUAUCCUUUCACGUCUUGACCCUCCCGAGGCUGAAGUGCAAAUUGAUAGGUUGAUCACAGGCAG |
| | | ACUUCAAAGUUUGCAGACAUAUGUGACUCAACAAUUAAUUAGAGCUGCAGAAAUCAGAGCUUCUGCUAAUCUUGCU |
| | | GCUACUAAAAUGUCAGAGUGUGUACUUGGACAAUCAAAAAGAGUUGAUUUUUGUGGAAAGGGCUAUCAUCUUAUG |
| | | UCCUUCCCUCAGUCAGCACCUCAUGGUGUAGUCUUCUUGCAUGUGACUUAUGUCCCUGCACAAGAAAAGAACUUC |
| | | ACAACUGCUCCUGCCAUUUGUCAUGAUGGAAAAGCACACUUUCCUCGUGAAGGUGUCUUUGUUUCAAAUGGCACA |
| | | CACUGGUUUGUAACACAAAGGAAUUUUUAUGAACCACAAAUCAUUACUACAGACAACACAUUUGUGUCUGGUAACU |
| | | GUGAUGUUGUAAUAGGAAUUGUCAACAACACAGUUUAUGAUCCUUUGCAACCUGAAUUAGACUCAUUCAAGGAGG |
| | | AGUUAGAUAAAUAUUUUAAGAAUCAUACAUCACCAGAUGUUGAUUUAGGUGACAUCUCUGGCAUUAAUGCUUCAGU |
| | | UGUAAACAUUCAAAAAGAAAUUGACCGCCUCAAUGAGGUUGCCAAGAAUUUAAAUGAAUCUCUCAUCGAUCUCCAA |
| | | GAACUUGGAAAGUAUGAGCAGUAUAUAAAAUGGCCAUGGUACAUUUGGCUAGGUUUUAUAGCUGGCUUGAUUGCC |
| | | AUAGUAAUGGUGACAAUUAUGCUUUGCUGUAUGACCAGUUGCUGUAGUUGUCUCAAGGGCUGUUGUUCUUGUGG |
| | | AUCCUGCUGCAAAUUUGAUGAAGACGACUCUGAGCCAGUGCUCAAAGGAGUCAAAUUACAUUACACA |
| 163 | P57 | AUGGGAGUCAAAGUUCUGUUUGCCCUGAUCUGCAUCGCUGUGGCCGAGGCCAAGAGAGUCCAACCAACAGAAUCU |
| | | AUUGUUAGAUUUCCUAAUAUUACAAACUUGUGCCCUUUUGGUGAAGUUUUUAACGCCACCAGAUUUGCAUCCGUG |
| | | UAUGCUUGGAACAGGAAGAGAAUCAGCAACUGUGUUGCUGAUUAUUCUGUCCUAUAUAAUUCCGCAUCAUUUUCC |
| | | ACUUUUAAGUGUUAUGGAGUGUCUCCUACUAAAUUAAAUGAUCUCUGCUUUACUAAUGUCUAUGCAGAUUCAUUU |
| | | GUAAUUAGAGGUGAUGAAGUCAGACAAAUCGCUCCAGGGCAAACUGGAAAGAUUGCUGAUUAUAAUUAUAAAUUAC |
| | | CAGAUGAUUUUACAGGCUGCGUUAUAGCUUGGAAUUCUAACAAUCUUGAUUCUAAGGUUGGUGGUAAUUAUAAUU |
| | | ACCUGUAUAGAUUGUUUAGGAAGUCUAAUCUCAAACCUUUUGAGAGAGAUAUUUCAACUGAAAUCUAUCAGGCCG |
| | | GUAGCACACCUUGUAAUGGUGUUGAAGGUUUUAAUUGUUACUUUCCUUUACAAUCAUAUGGUUUCCAACCCACUA |
| | | AUGGUGUUGGUUACCAACCAUACAGAGUAGUAGUACUUUCUUUUGAACUUCUACAUGCACCAGCAACUGUUUGUG |
| | | GACCUAAAAAGUCUACUAAUUUGGUUAAAAACAAAUGUGUCAAUUUCGGGUAUAUCCCUGAAGCCCCCAGGGACG |
| | | GCCAGGCUUACGUCAGAAAGGAUGGAGAGUGGGUGCUCUUGAGCACCUUCCUGUAA |
| 164 | P58 | AUGUUUGUUUUUCUUGUUUUAUUGCCACUAGUCUCUAGUCAGUGUGUUAAUCUUACAACCAGAACUCAAUUACCC |
| | | CCUGCAUACACUAAUUCUUUCACACGUGGUGUUUAUUACCCUGACAAAGUUUUCAGAUCCUCAGUUUUACAUUCA |
| | | ACUCAGGAUUUGUUCUUACCUUUCUUUUCCAAUGUUACUUGGUUCCAUGCUAUACAUGUCUCUGGGACCAAUGGU |
| | | ACUAAGAGGUUUGAUAACCCUGUCCUACCAUUUAAUGAUGGUGUUUAUUUUGCUUCCACUGAGAAGUCUAACAUA |
| | | AUAAGAGGCUGGAUCUUUGGUACUACUUUAGAUUCGAAGACCCAGUCCCUACUUAUUGUUAAUAACGCUACUAAU |
| | | GUUGUUAUUAAAGUCUGUGAAUUUCAAUUUUGUAAUGAUCCAUUUUUGGGUGUUUAUUACCACAAAAACAACAAAA |
| | | GUUGGAUGGAAAGUGAGUUCAGAGUUUAUUCUAGUGCGAAUAAUUGCACUUUUGAAUAUGUCUCUCAGCCUUUUC |
| | | UUAUGGACCUUGAAGGAAAACAGGGUAAUUUCAAAAAUCUUAGGGAAUUUGUGUUUAAGAAUAUUGAUGGUUAUU |
| | | UUAAAAUAUAUUCUAAGCACACGCCUAUUAAUUUAGUGCGUGAUCUCCCUCAGGGUUUUUCGGCUUUAGAACCAU |
| | | UGGUAGAUUUGCCAAUAGGUAUUAACAUCACUAGGUUUCAAACUUUACUUGCUUUACAUAGAAGUUAUUUGACUC |
| | | CUGGUGAUUCUUCUUCAGGUUGGACAGCUGGUGCUGCAGCUUAUUAUGUGGGUUAUCUUCAACCUAGGACUUUU |
| | | CUAUUAAAAUAUAAUGAAAAUGGAACCAUUACAGAUGCUGUAGACUGUGCACUUGACCCUCUCUCAGAAACAAAGU |
| | | GUACGUUGAAAUCCUUCACUGUAGAAAAAGGAAUCUAUCAAACUUCUAACUUUAGAGUCCAACCAACAGAAUCUAU |
| | | UGUUAGAUUUCCUAAUAUUACAAACUUGUGCCCUUUUGGUGAAGUUUUUAACGCCACCAGAUUUGCAUCCGUGUA |
| | | UGCUUGGAACAGGAAGAGAAUCAGCAACUGUGUUGCUGAUUAUUCUGUCCUAUAUAAUUCCGCAUCAUUUUCCAC |
| | | UUUUAAGUGUUAUGGAGUGUCUCCUACUAAAUUAAAUGAUCUCUGCUUUACUAAUGUCUAUGCAGAUUCAUUUGU |
| | | AAUUAGAGGUGAUGAAGUCAGACAAAUCGCUCCAGGGCAAACUGGAAAGAUUGCUGAUUAUAAUUAUAAAUUACCA |
| | | GAUGAUUUUACAGGCUGCGUUAUAGCUUGGAAUUCUAACAAUCUUGAUUCUAAGGUUGGUGGUAAUUAUAAUUAC |
| | | CUGUAUAGAUUGUUUAGGAAGUCUAAUCUCAAACCUUUUGAGAGAGAUAUUUCAACUGAAAUCUAUCAGGCCGGU |
| | | AGCACACCUUGUAAUGGUGUUGAAGGUUUUAAUUGUUACUUUCCUUUACAAUCAUAUGGUUUCCAACCCACUAAU |
| | | GGUGUUGGUUACCAACCAUACAGAGUAGUAGUACUUUCUUUUGAACUUCUACAUGCACCAGCAACUGUUUGUGGA |
| | | CCUAAAAAGUCUACUAAUUUGGUUAAAAACAAAUGUGUCAAUUUCAACUUCAAUGGUUUAACAGGCACAGGUGUUC |
| | | UUACUGAGUCUAACAAAAAGUUUCUGCCUUUCCAACAAUUUGGCAGAGACAUUGCUGACACUACUGAUGCUGUCC |
| | | GUGAUCCACAGACACUUGAGAUUCUUGACAUUACACCAUGUUCUUUUGGUGGUGUCAGUGUUAUAACACCAGGAA |
| | | CAAAUACUUCUAACCAGGUUGCUGUUCUUUAUCAGGGUGUUAACUGCACAGAAGUCCCUGUUGCUAUUCAUGCAG |
| | | AUCAACUUACUCCUACUUGGCGUGUUUAUUCUACAGGUUCUAAUGUUUUUCAAACACGUGCAGGCUGUUUAAUAG |
| | | GGGCUGAACAUGUCAACAACUCAUAUGAGUGUGACAUACCCAUUGGUGCAGGUAUAUGCGCUAGUUAUCAGACUC |
| | | AGACUAAUUCUCCUGGCAGCGCCAGCAGUGUAGCUAGUCAAUCCAUCAUUGCCUACACUAUGUCACUUGGUGCAG |
| | | AAAAUUCAGUUGCUUACUCUAAUAACUCUAUUGCCAUACCCACAAAUUUUACUAUUAGUGUUACCACAGAAAUUCU |
| | | ACCAGUGUCUAUGACCAAGACAUCAGUAGAUUGUACAAUGUACAUUUGUGGUGAUUCAACUGAAUGCAGCAAUCU |
| | | UUUGUUGCAAUAUGGCAGUUUUUGUACACAAUUAAACCGUGCUUUAACUGGGAUAGCUGUUGAACAAGACAAAAA |
| | | CACCCAAGAAGUUUUUGCACAAGUCAAACAAAUUUACAAAACACCACCAAUUAAAGAUUUUGGUGGUUUUAAUUUU |
| | | UCACAAAUAUUACCAGAUCCAUCAAAACCAAGCAAGAGGUCAUUUAUUGAAGAUCUACUUUUCAACAAAGUGACAC |
| | | UUGCAGAUGCUGGCUUCAUCAAACAAUAUGGUGAUUGCCUUGGUGAUAUUGCUGCUAGGGACCUCAUUUGUGCA |
| | | CAAAAGUUUAACGGCCUUACUGUUUUGCCACCUUUGCUCACAGAUGAAAUGAUUGCUCAAUACACUUCUGCACUG |
| | | UUAGCGGGUACAAUCACUUCUGGUUGGACCUUUGGUGCAGGUGCUGCAUUACAAAUACCAUUUGCUAUGCAAAUG |
| | | GCUUAUAGGUUUAAUGGUAUUGGAGUUACACAGAAUGUUCUCUAUGAGAACCAAAAAUUGAUUGCCAACCAAUUUA |
| | | AUAGUGCCAUUGGCAAAAUUCAAGACUCACUUUCUUCCACAGCAAGUGCACUUGGAAAACUUCAAGAUGUGGUCA |
| | | ACCAAAAUGCACAAGCUUUAAACACGCUUGUUAAACAACUUAGCUCCAAUUUUGGUGCAAUUUCAAGUGUUUUAAA |
| | | UGAUAUCCUUUCACGUCUUGACCCUCCCGAGGCUGAAGUGCAAAUUGAUAGGUUGAUCACAGGCAGACUUCAAAG |
| | | UUUGCAGACAUAUGUGACUCAACAAUUAAUUAGAGCUGCAGAAAUCAGAGCUUCUGCUAAUCUUGCUGCUACUAAA |
| | | AUGUCAGAGUGUGUACUUGGACAAUCAAAAAGAGUUGAUUUUUGUGGAAAGGGCUAUCAUCUUAUGUCCUUCCCU |
| | | CAGUCAGCACCUCAUGGUGUAGUCUUCUUGCAUGUGACUUAUGUCCCUGCACAAGAAAAGAACUUCACAACUGCU |
| | | CCUGCCAUUUGUCAUGAUGGAAAAGCACACUUUCCUCGUGAAGGUGUCUUUGUUUCAAAUGGCACACACUGGUUU |
| | | GUAACACAAAGGAAUUUUUAUGAACCACAAAUCAUUACUACAGACAACACAUUUGUGUCUGGUAACUGUGAUGUUG |
| | | UAAUAGGAAUUGUCAACAACACAGUUUAUGAUCCUUUGCAACCUGAAUUAGACUCAUUCAAGGAGGAGUUAGAUAA |
| | | AUAUUUUAAGAAUCAUACAUCACCAGAUGUUGAUUUAGGUGACAUCUCUGGCAUUAAUGCUUCAGUUGUAAACAUU |
| | | CAAAAAGAAAUUGACCGCCUCAAUGAGGUUGCCAAGAAUUUAAAUGAAUCUCUCAUCGAUCUCCAAGAACUUGGAA |
| | | AGUAUGAGCAGUAUAUAAAAUGGCCAUGGUACAUUUGGCUAGGUUUUAUAGCUGGCUUGAUUGCCAUAGUAAUGG |
| | | UGACAAUUAUGCUUUGCUGUAUGACCAGUUGCUGUAGUUGUCUCAAGGGCUGUUGUUCUUGUGGAUCCUGCUGC |
| | | AAAUUUGAUGAAGACGACUCUGAGCCAGUGCUCAAAGGAGUCAAAUUACAUUACACA |
| 165 | 2A2361 | AUGGGAGUCAAAGUUCUGUUUGCCCUGAUCUGCAUUGCUGUGGCCGAGGCCAAGAGAGUCCAACCAACAGAAUCU |
| | | AUUGUUAGAUUUCCUAAUAUUACAAACUUGUGCCCUUUUGGUGAAGUUUUUAACGCCACCAGAUUUGCAUCCGUG |
| | | UAUGCUUGGAACAGGAAGAGAAUCAGCAACUGUGUUGCUGAUUAUUCUGUCCUAUAUAAUUCCGCAUCAUUUUCC |
| | | ACUUUUAAGUGUUAUGGAGUGUCUCCUACUAAAUUAAAUGAUCUCUGCUUUACUAAUGUCUAUGCAGAUUCAUUU |
| | | GUAAUUAGAGGUGAUGAAGUCAGACAAAUCGCUCCAGGGCAAACUGGAAAGAUUGCUGAUUAUAAUUAUAAAUUAC |
| | | CAGAUGAUUUUACAGGCUGCGUUAUAGCUUGGAAUUCUAACAAUCUUGAUUCUAAGGUUGGUGGUAAUUAUAAUU |
| | | ACCUGUAUAGAUUGUUUAGGAAGUCUAAUCUCAAACCUUUUGAGAGAGAUAUUUCAACUGAAAUCUAUCAGGCCG |
| | | GUAGCACACCUUGUAAUGGUGUUGAAGGUUUUAAUUGUUACUUUCCUUUACAAUCAUAUGGUUUCCAACCCACUA |
| | | AUGGUGUUGGUUACCAACCAUACAGAGUAGUAGUACUUUCUUUUGAACUUCUACAUGCACCAGCAACUGUUUGUG |
| | | GACCUAAAAAGUCUACUAAUUUGGUUAAAAACAAAUGUGUCAAUUUCGGAGGAGGAGGAAGCGGAUACAUCCCCG |
| | | AGGCCCCCCGGGACGGCCAGGCCUACGUGCGGAAGGACGGCGAGUGGGUGCUGCUCAGCACCUUCCUG |
| 166 | 2A3361 | AUGGGAGUCAAAGUUCUGUUUGCCCUGAUCUGCAUUGCUGUGGCCGAGGCCAAGAGAGUCCAACCAACAGAAUCU |
| | | AUUGUUAGAUUUCCUAAUAUUACAAACUUGUGCCCUUUUGGUGAAGUUUUUAACGCCACCAGAUUUGCAUCCGUG |
| | | UAUGCUUGGAACAGGAAGAGAAUCAGCAACUGUGUUGCUGAUUAUUCUGUCCUAUAUAAUUCCGCAUCAUUUUCC |
| | | ACUUUUAAGUGUUAUGGAGUGUCUCCUACUAAAUUAAAUGAUCUCUGCUUUACUAAUGUCUAUGCAGAUUCAUUU |
| | | GUAAUUAGAGGUGAUGAAGUCAGACAAAUCGCUCCAGGGCAAACUGGAAAGAUUGCUGAUUAUAAUUAUAAAUUAC |
| | | CAGAUGAUUUUACAGGCUGCGUUAUAGCUUGGAAUUCUAACAAUCUUGAUUCUAAGGUUGGUGGUAAUUAUAAUU |
| | | ACCUGUAUAGAUUGUUUAGGAAGUCUAAUCUCAAACCUUUUGAGAGAGAUAUUUCAACUGAAAUCUAUCAGGCCG |
| | | GUAGCACACCUUGUAAUGGUGUUGAAGGUUUUAAUUGUUACUUUCCUUUACAAUCAUAUGGUUUCCAACCCACUA |
| | | AUGGUGUUGGUUACCAACCAUACAGAGUAGUAGUACUUUCUUUUGAACUUCUACAUGCACCAGCAACUGUUUGUG |
| | | GACCUAAAAAGUCUACUAAUUUGGUUAAAAACAAAUGUGUCAAUUUCGGAGGAGGAGGAAGCGGAUACAUCCCCG |
| | | AGGCCCCCCGGGACGGCCAGGCCUACGUGCGGAAGGACGGCGAGUGGGUGCUGCUCAGCACCUUCCUGGGAGG |
| | | AGGAGGAAGCAGAGUCCAACCAACAGAAUCUAUUGUUAGAUUUCCUAAUAUUACAAACUUGUGCCCUUUUGGUGA |
| | | AGUUUUUAACGCCACCAGAUUUGCAUCCGUGUAUGCUUGGAACAGGAAGAGAAUCAGCAACUGUGUUGCUGAUUA |
| | | UUCUGUCCUAUAUAAUUCCGCAUCAUUUUCCACUUUUAAGUGUUAUGGAGUGUCUCCUACUAAAUUAAAUGAUCU |
| | | CUGCUUUACUAAUGUCUAUGCAGAUUCAUUUGUAAUUAGAGGUGAUGAAGUCAGACAAAUCGCUCCAGGGCAAAC |
| | | UGGAAAGAUUGCUGAUUAUAAUUAUAAAUUACCAGAUGAUUUUACAGGCUGCGUUAUAGCUUGGAAUUCUAACAAU |
| | | CUUGAUUCUAAGGUUGGUGGUAAUUAUAAUUACCUGUAUAGAUUGUUUAGGAAGUCUAAUCUCAAACCUUUUGAG |
| | | AGAGAUAUUUCAACUGAAAUCUAUCAGGCCGGUAGCACACCUUGUAAUGGUGUUGAAGGUUUUAAUUGUUACUUU |
| | | CCUUUACAAUCAUAUGGUUUCCAACCCACUAAUGGUGUUGGUUACCAACCAUACAGAGUAGUAGUACUUUUUUU |
| | | GAACUUCUACAUGCACCAGCAACUGUUUGUGGACCUAAAAAGUCUACUAAUUUGGUUAAAAACAAAUGUGUCAAUU |
| | | UC |
| 167 | 2A6361 | AUGGGAGUCAAAGUUCUGUUUGCCCUGAUCUGCAUUGCUGUGGCCGAGGCCAAGAGAGUCCAACCAACAGAAUCU |
| | | AUUGUUAGAUUUCCUAAUAUUACAAACUUGUGCCCUUUUGGUGAAGUUUUUAACGCCACCAGAUUUGCAUCCGUG |
| | | UAUGCUUGGAACAGGAAGAGAAUCAGCAACUGUGUUGCUGAUUAUUCUGUCCUAUAUAAUUCCGCAUCAUUUUCC |
| | | ACUUUUAAGUGUUAUGGAGUGUCUCCUACUAAAUUAAAUGAUCUCUGCUUUACUAAUGUCUAUGCAGAUUCAUUU |
| | | GUAAUUAGAGGUGAUGAAGUCAGACAAAUCGCUCCAGGGCAAACUGGAAAGAUUGCUGAUUAUAAUUAUAAAUUAC |
| | | CAGAUGAUUUUACAGGCUGCGUUAUAGCUUGGAAUUCUAACAAUCUUGAUUCUAAGGUUGGUGGUAAUUAUAAUU |
| | | ACCUGUAUAGAUUGUUUAGGAAGUCUAAUCUCAAACCUUUUGAGAGAGAUAUUUCAACUGAAAUCUAUCAGGCCG |
| | | GUAGCACACCUUGUAAUGGUGUUGAAGGUUUUAAUUGUUACUUUCCUUUACAAUCAUAUGGUUUCCAACCCACUA |
| | | AUGGUGUUGGUUACCAACCAUACAGAGUAGUAGUACUUUCUUUUGAACUUCUACAUGCACCAGCAACUGUUUGUG |
| | | GACCUAAAAAGUCUACUAAUUUGGUUAAAAACAAAUGUGUCAAUUUCGACAUCAUUAAGCUGCUCAACGAGCAGGU |
| | | GAACAAGGAGAUGAACAGCUCCAACCUGUACAUGAGCAUGAGCAGCUGGUGCUACACCCACAGCCUGGACGGCGC |
| | | CGGCCUGUUCCUGUUUGACCACGCCGCUGAGGAAUACGAGCACGCCAAGAAACUGAUCGUGUUCCUGAACGAGAA |
| | | CAACGUGCCCGUGCAGCUGACCAGCAUCAGCGCCCCCGAGCACAAGUUCGAGAGCCUGACCCAGAUCUUCCAGAA |
| | | GGCCUACGAGCACGAGCAGCACAUCAGCGAGAGCAUCAACAACAUCGUCGACCACGCCAUCAAGGGCAAGGACCA |
| | | CGCCACCUUCAACUUCCUGCAGUGGUACGUGAGCGAGCAGCACGAGGAGGAGGUGCUGUUCAAGGACAUCCUGG |
| | | ACAAGAUCGAGCUGAUCGGCAACGAGAACCACGGCCUGUACCUGGCCGACCAGUACGUGAAGGGCAUCGCCAAGA |
| | | GCCGCAAAAGU |
| 168 | 2A7361 | AUGGGAGUCAAAGUUCUGUUUGCCCUGAUCUGCAUUGCUGUGGCCGAGGCCAAGAGAGUCCAACCAACAGAAUCU |
| | | AUUGUUAGAUUUCCUAAUAUUACAAACUUGUGCCCUUUUGGUGAAGUUUUUAACGCCACCAGAUUUGCAUCCGUG |
| | | UAUGCUUGGAACAGGAAGAGAAUCAGCAACUGUGUUGCUGAUUAUUCUGUCCUAUAUAAUUCCGCAUCAUUUUCC |
| | | ACUUUUAAGUGUUAUGGAGUGUCUCCUACUAAAUUAAAUGAUCUCUGCUUUACUAAUGUCUAUGCAGAUUCAUUU |
| | | GUAAUUAGAGGUGAUGAAGUCAGACAAAUCGCUCCAGGGCAAACUGGAAAGAUUGCUGAUUAUAAUUAUAAAUUAC |
| | | CAGAUGAUUUUACAGGCUGCGUUAUAGCUUGGAAUUCUAACAAUCUUGAUUCUAAGGUUGGUGGUAAUUAUAAUU |
| | | ACCUGUAUAGAUUGUUUAGGAAGUCUAAUCUCAAACCUUUUGAGAGAGAUAUUUCAACUGAAAUCUAUCAGGCCG |
| | | GUAGCACACCUUGUAAUGGUGUUGAAGGUUUUAAUUGUUACUUUCCUUUACAAUCAUAUGGUUUCCAACCCACUA |
| | | AUGGUGUUGGUUACCAACCAUACAGAGUAGUAGUACUUUCUUUUGAACUUCUACAUGCACCAGCAACUGUUUGUG |
| | | GACCUAAAAAGUCUACUAAUUUGGUUAAAAACAAAUGUGUCAAUUUCGGAGGAGGAGGAAGCGACAUCAUUAAGCU |
| | | GCUCAACGAGCAGGUGAACAAGGAGAUGAACAGCUCCAACCUGUACAUGAGCAUGAGCAGCUGGUGCUACACCCA |
| | | CAGCCUGGACGGCGCCGGCCUGUUCCUGUUUGACCACGCCGCUGAGGAAUACGAGCACGCCAAGAAACUGAUCG |
| | | UGUUCCUGAACGAGAACAACGUGCCCGUGCAGCUGACCAGCAUCAGCGCCCCCGAGCACAAGUUCGAGAGCCUGA |
| | | CCCAGAUCUUCCAGAAGGCCUACGAGCACGAGCAGCACAUCAGCGAGAGCAUCAACAACAUCGUCGACCACGCCA |
| | | UCAAGGGCAAGGACCACGCCACCUUCAACUUCCUGCAGUGGUACGUGAGCGAGCAGCACGAGGAGGAGGUGCUG |
| | | UUCAAGGACAUCCUGGACAAGAUCGAGCUGAUCGGCAACGAGAACCACGGCCUGUACCUGGCCGACCAGUACGUG |
| | | AAGGGCAUCGCCAAGAGCCGCAAAAGU |
| 169 | 2A8361 | AUGGGAGUCAAAGUUCUGUUUGCCCUGAUCUGCAUUGCUGUGGCCGAGGCCAAGAGAGUCCAACCAACAGAAUCU |
| | | AUUGUUAGAUUUCCUAAUAUUACAAACUUGUGCCCUUUUGGUGAAGUUUUUAACGCCACCAGAUUUGCAUCCGUG |
| | | UAUGCUUGGAACAGGAAGAGAAUCAGCAACUGUGUUGCUGAUUAUUCUGUCCUAUAUAAUUCCGCAUCAUUUUCC |
| | | ACUUUUAAGUGUUAUGGAGUGUCUCCUACUAAAUUAAAUGAUCUCUGCUUUACUAAUGUCUAUGCAGAUUCAUUU |
| | | GUAAUUAGAGGUGAUGAAGUCAGACAAAUCGCUCCAGGGCAAACUGGAAAGAUUGCUGAUUAUAAUUAUAAAUUAC |
| | | CAGAUGAUUUUACAGGCUGCGUUAUAGCUUGGAAUUCUAACAAUCUUGAUUCUAAGGUUGGUGGUAAUUAUAAUU |
| | | ACCUGUAUAGAUUGUUUAGGAAGUCUAAUCUCAAACCUUUUGAGAGAGAUAUUUCAACUGAAAUCUAUCAGGCCG |
| | | GUAGCACACCUUGUAAUGGUGUUGAAGGUUUUAAUUGUUACUUUCCUUUACAAUCAUAUGGUUUCCAACCCACUA |
| | | AUGGUGUUGGUUACCAACCAUACAGAGUAGUAGUACUUUCUUUUGAACUUCUACAUGCACCAGCAACUGUUUGUG |
| | | GACCUAAAAAGUCUACUAAUUUGGUUAAAAACAAAUGUGUCAAUUUCCCCGGCAGCGGCUACAUCCCCGAGGCCC |
| | | CCCGGGACGGCCAGGCCUACGUGCGGAAGGACGGCGAGUGGGUGCUGCUCAGCACCUUCCUGAGCGGCCGGAG |
| | | CGGCGGAGACAUCAUUAAGCUGCUGAACGAGCAGGUGAACAAGGAGAUGAACAGCUCCAACCUGUACAUGAGCAU |
| | | GAGCAGCUGGUGCUACACCCACAGCCUGGACGGCGCCGGCCUGUUCCUGUUUGACCACGCCGCUGAGGAAUACG |
| | | AGCACGCCAAGAAACUGAUCGUGUUCCUGAACGAGAACAACGUGCCCGUGCAGCUGACCAGCAUCAGCGCCCCCG |
| | | AGCACAAGUUCGAGAGCCUGACCCAGAUCUUCCAGAAGGCCUACGAGCACGAGCAGCACAUCAGCGAGAGCAUCA |
| | | ACAACAUCGUCGACCACGCCAUCAAGGGCAAGGACCACGCCACCUUCAACUUCCUGCAGUGGUACGUGAGCGAGC |
| | | AGCACGAGGAGGAGGUGCUGUUCAAGGACAUCCUGGACAAGAUCGAGCUGAUCGGCAACGAGAACCACGGCCUG |
| | | UACCUGGCCGACCAGUACGUGAAGGGCAUCGCCAAGAGCCGCAAAAGU |
| 170 | 2A9361 | AUGGGAGUCAAAGUUCUGUUUGCCCUGAUCUGCAUUGCUGUGGCCGAGGCCAAGAGAGUCCAACCAACAGAAUCU |
| | | AUUGUUAGAUUUCCUAAUAUUACAAACUUGUGCCCUUUUGGUGAAGUUUUUAACGCCACCAGAUUUGCAUCCGUG |
| | | UAUGCUUGGAACAGGAAGAGAAUCAGCAACUGUGUUGCUGAUUAUUCUGUCCUAUAUAAUUCCGCAUCAUUUUCC |
| | | ACUUUUAAGUGUUAUGGAGUGUCUCCUACUAAAUUAAAUGAUCUCUGCUUUACUAAUGUCUAUGCAGAUUCAUUU |
| | | GUAAUUAGAGGUGAUGAAGUCAGACAAAUCGCUCCAGGGCAAACUGGAAAGAUUGCUGAUUAUAAUUAUAAAUUAC |
| | | CAGAUGAUUUUACAGGCUGCGUUAUAGCUUGGAAUUCUAACAAUCUUGAUUCUAAGGUUGGUGGUAAUUAUAAUU |
| | | ACCUGUAUAGAUUGUUUAGGAAGUCUAAUCUCAAACCUUUUGAGAGAGAUAUUUCAACUGAAAUCUAUCAGGCCG |
| | | GUAGCACACCUUGUAAUGGUGUUGAAGGUUUUAAUUGUUACUUUCCUUUACAAUCAUAUGGUUUCCAACCCACUA |
| | | AUGGUGUUGGUUACCAACCAUACAGAGUAGUAGUACUUUCUUUUGAACUUCUACAUGCACCAGCAACUGUUUGUG |
| | | GACCUAAAAAGUCUACUAAUUUGGUUAAAAACAAAUGUGUCAAUUUCAUCAACCACGUGGGCGGAACCGGCGGCG |
| | | CCAUCAUGGCCCCCGUGGCCGUGACCCGGCAGCUGGUGGGCAGC |
| 171 | 2A0461 | AUGGGAGUCAAAGUUCUGUUUGCCCUGAUCUGCAUUGCUGUGGCCGAGGCCAAGAGAGUCCAACCAACAGAAUCU |
| | | AUUGUUAGAUUUCCUAAUAUUACAAACUUGUGCCCUUUUGGUGAAGUUUUUAACGCCACCAGAUUUGCAUCCGUG |
| | | UAUGCUUGGAACAGGAAGAGAAUCAGCAACUGUGUUGCUGAUUAUUCUGUCCUAUAUAAUUCCGCAUCAUUUUCC |
| | | ACUUUUAAGUGUUAUGGAGUGUCUCCUACUAAAUUAAAUGAUCUCUGCUUUACUAAUGUCUAUGCAGAUUCAUUU |
| | | GUAAUUAGAGGUGAUGAAGUCAGACAAAUCGCUCCAGGGCAAACUGGAAAGAUUGCUGAUUAUAAUUAUAAAUUAC |
| | | CAGAUGAUUUUACAGGCUGCGUUAUAGCUUGGAAUUCUAACAAUCUUGAUUCUAAGGUUGGUGGUAAUUAUAAUU |
| | | ACCUGUAUAGAUUGUUUAGGAAGUCUAAUCUCAAACCUUUUGAGAGAGAUAUUUCAACUGAAAUCUAUCAGGCCG |
| | | GUAGCACACCUUGUAAUGGUGUUGAAGGUUUUAAUUGUUACUUUCCUUUACAAUCAUAUGGUUUCCAACCCACUA |
| | | AUGGUGUUGGUUACCAACCAUACAGAGUAGUAGUACUUUCUUUUGAACUUCUACAUGCACCAGCAACUGUUUGUG |
| | | GACCUAAAAAGUCUACUAAUUUGGUUAAAAACAAAUGUGUCAAUUUCGGAGGAGGAGGAAGCAUCAACCACGUGG |
| | | GCGGAACCGGCGGCGCCAUCAUGGCCCCCGUGGCCGUGACCCGGCAGCUGGUGGGCAGC |
| 172 | 2A1461 | AUGGGAGUCAAAGUUCUGUUUGCCCUGAUCUGCAUUGCUGUGGCCGAGGCCAAGAUCAACCACGUGGGGGAAC |
| | | CGGCGGCGCCAUCAUGGCCCCCGUGGCCGUGACCCGGCAGCUGGUGGGCAGCAGAGUCCAACCAACAGAAUCUA |
| | | UUGUUAGAUUUCCUAAUAUUACAAACUUGUGCCCUUUUGGUGAAGUUUUUAACGCCACCAGAUUUGCAUCCGUGU |
| | | AUGCUUGGAACAGGAAGAGAAUCAGCAACUGUGUUGCUGAUUAUUCUGUCCUAUAUAAUUCCGCAUCAUUUUCCA |
| | | CUUUUAAGUGUUAUGGAGUGUCUCCUACUAAAUUAAAUGAUCUCUGCUUUACUAAUGUCUAUGCAGAUUCAUUUG |
| | | UAAUUAGAGGUGAUGAAGUCAGACAAAUCGCUCCAGGGCAAACUGGAAAGAUUGCUGAUUAUAAUUAUAAAUUACC |
| | | AGAUGAUUUUACAGGCUGCGUUAUAGCUUGGAAUUCUAACAAUCUUGAUUCUAAGGUUGGUGGUAAUUAUAAUUA |
| | | CCUGUAUAGAUUGUUUAGGAAGUCUAAUCUCAAACCUUUUGAGAGAGAUAUUUCAACUGAAAUCUAUCAGGCCGG |
| | | UAGCACACCUUGUAAUGGUGUUGAAGGUUUUAAUUGUUACUUUCCUUUACAAUCAUAUGGUUUCCAACCCACUAA |
| | | UGGUGUUGGUUACCAACCAUACAGAGUAGUAGUACUUUCUUUUGAACUUCUACAUGCACCAGCAACUGUUUGUGG |
| | | ACCUAAAAAGUCUACUAAUUUGGUUAAAAACAAAUGUGUCAAUUUC |
| 173 | 2A2461 | AUGGGAGUCAAAGUUCUGUUUGCCCUGAUCUGCAUUGCUGUGGCCGAGGCCAAGAUCAACCACGUGGGGGAAC |
| | | CGGCGGCGCCAUCAUGGCCCCCGUGGCCGUGACCCGGCAGCUGGUGGGCAGCGGAGGAGGAGGAAGCAGAGUC |
| | | CAACCAACAGAAUCUAUUGUUAGAUUUCCUAAUAUUACAAACUUGUGCCCUUUUGGUGAAGUUUUUAACGCCACCA |
| | | GAUUUGCAUCCGUGUAUGCUUGGAACAGGAAGAGAAUCAGCAACUGUGUUGCUGAUUAUUCUGUCCUAUAUAAUU |
| | | CCGCAUCAUUUUCCACUUUUAAGUGUUAUGGAGUGUCUCCUACUAAAUUAAAUGAUCUCUGCUUUACUAAUGUCU |
| | | AUGCAGAUUCAUUUGUAAUUAGAGGUGAUGAAGUCAGACAAAUCGCUCCAGGGCAAACUGGAAAGAUUGCUGAUU |
| | | AUAAUUAUAAAUUACCAGAUGAUUUUACAGGCUGCGUUAUAGCUUGGAAUUCUAACAAUCUUGAUUCUAAGGUUG |
| | | GUGGUAAUUAUAAUUACCUGUAUAGAUUGUUUAGGAAGUCUAAUCUCAAACCUUUUGAGAGAGAUAUUUCAACUG |
| | | AAAUCUAUCAGGCCGGUAGCACACCUUGUAAUGGUGUUGAAGGUUUUAAUUGUUACUUUCCUUUACAAUCAUAUG |
| | | GUUUCCAACCCACUAAUGGUGUUGGUUACCAACCAUACAGAGUAGUAGUACUUUCUUUUGAACUUCUACAUGCAC |
| | | CAGCAACUGUUUGUGGACCUAAAAAGUCUACUAAUUUGGUUAAAAACAAAUGUGUCAAUUUC |
| 174 | 2A7520 | AUGGGAGUCAAAGUUCUGUUUGCCCUGAUCUGCAUUGCUGUGGCCGAGGCCAAGAGAGUCCAACCAACAGAAUCU |
| | | AUUGUUAGAUUUCCUAAUAUUACAAACUUGUGCCCUUUUGGUGAAGUUUUUAACGCCACCAGAUUUGCAUCCGUG |
| | | UAUGCUUGGAACAGGAAGAGAAUCAGCAACUGUGUUGCUGAUUAUUCUGUCCUAUAUAAUUCCGCAUCAUUUUCC |
| | | ACUUUUAAGUGUUAUGGAGUGUCUCCUACUAAAUUAAAUGAUCUCUGCUUUACUAAUGUCUAUGCAGAUUCAUUU |
| | | GUAAUUAGAGGUGAUGAAGUCAGACAAAUCGCUCCAGGGCAAACUGGAAAGAUUGCUGAUUAUAAUUAUAAAUUAC |
| | | CAGAUGAUUUUACAGGCUGCGUUAUAGCUUGGAAUUCUAACAAUCUUGAUUCUAAGGUUGGUGGUAAUUAUAAUU |
| | | ACCUGUAUAGAUUGUUUAGGAAGUCUAAUCUCAAACCUUUUGAGAGAGAUAUUUCAACUGAAAUCUAUCAGGCCG |
| | | GUAGCACACCUUGUAAUGGUGUUGAAGGUUUUAAUUGUUACUUUCCUUUACAAUCAUAUGGUUUCCAACCCACUA |
| | | AUGGUGUUGGUUACCAACCAUACAGAGUAGUAGUACUUUCUUUUGAACUUCUACAUGCACCAGCAACUGUUUGUG |
| | | GACCUAAAAAGUCUACUAAUUUGGUUAAAAACAAAUGUGUCAAUUUCGGGUAUAUCCCUGAAGCCCCCAGGGACG |
| | | GCCAGGCUUACGUCAGAAAGGAUGGAGAGUGGGUGCUCUUGAGCACCUUCCUGUAA |
| 217 | IL-12 | atggctccccaaaagctgaccatcagctggttcgccatcgtgctgctcgtgagccccctgatggccatgtgggagctggagaaggacgtgtacgtggtcgaggtggactgga |
| | | cccccgacgcccccggcgaaacagtgaacctgacctgcgacacccccgaggaagacgatatcacctggacaagcgaccagcggcacggcgtgatcggcagcggca |
| | | agaccctgaccatcaccgtgaaggagttcctggacgccggccagtacacctgccacaagggcggagaaacactgagccacagccacctgctgttgcacaagaaagag |
| | | aacggcatctggagcaccgagatcctcaagaatttcaagaacaagaccttcctgaagtgcgaggcccccaactacagcggccggttcacctgcagctggctggtgcagcg |
| | | gaacatggacctgaagttcaacatcaagagctccagctctagccccgacagccgggccgtgacctgcggcatggccagcctgagcgccgagaaggtgaccctggacca |
| | | gcgggactacgagaagtacagcgtgagctgccaggaggacgtgacctgccctaccgctgaggaaacactgcccatcgagctggccctggaggcccggcagcaaaac |
| | | aagtacgagaactacagcaccagcttctttatccgggacatcattaagcccgacccccctaagaacctgcagatgaagcccctgaagaacagccaggtggaggtgagct |
| | | gggagtaccccgacagctggagcaccccccacagctacttcagcctgaagttcttcgtgcggatccagcggaagaaggagaagatgaaggaaacagaggagggctgc |
| | | aaccagaagggcgccttcctggtggaaaagacaagcacagaggtgcagtgcaagggcggcaacgtgtgcgtgcaggcccaggaccggtactataacagcagctgca |
| | | gcaagtgggcctgcgtgccctgccgggtgcggagtgtgcctggcgtcggcgtgcccggcgtgggccgggtgatccccgtgagcggccccgcccggtgcctgagccagag |
| | | ccggaacctgctgaagaccacagacgacatggtgaagaccgcccgggagaagctgaagcactacagctgcaccgccgaggacatcgaccacgaggacatcacccg |
| | | ggaccagaccagcaccctgaagacctgcctccccctggagctgcacaagaacgagagctgcctggccacccgggaaacaagctctaccacacggggcagctgcctgc |
| | | ctcctcagaagaccagcctgatgatgaccctgtgcctgggcagcatctacgaggacctgaagatgtaccagaccgagttccaggccatcaacgccgctctgcagaaccac |
| | | aaccaccagcagatcatcctggacaagggcatgctggtggccatcgacgagctgatgcagagcctgaaccacaacggcgaaacactgcggcagaagcccccagtgg |
| | | gcgaggccgacccctaccgggtgaagatgaagctgtgcatcctgctgcacgccttcagcacccgggtggtgaccatcaaccgggtgatgggctacctgagcagcgcc |
| 218 | IL12 | MCPQKLTISWFAIVLLVSPLMAMWELEKDVYVVEVDWTPDAPGETVNLTCDTPEEDDITWTSDQRHGVIGSGKTLTITVKEF |
| | | LDAGQYTCHKGGETLSHSHLLLHKKENGIWSTEILKNFKNKTFLKCEAPNYSGRFTCSWLVQRNMDLKFNIKSSSSSPDSR |
| | | AVTCGMASLSAEKVTLDQRDYEKYSVSCQEDVTCPTAEETLPIELALEARQQNKYENYSTSFFIRDIIKPDPPKNLQMKPLK |
| | | NSQVEVSWEYPDSWSTPHSYFSLKFFVRIQRKKEKMKETEEGCNQKGAFLVEKTSTEVQCKGGNVCVQAQDRYYNSSCS |
| | | KWACVPCRVRSVPGVGVPGVGRVIPVSGPARCLSQSRNLLKTTDDMVKTAREKLKHYSCTAEDIDHEDITRDQTSTLKTCL |
| | | PLELHKNESCLATRETSSTTRGSCLPPQKTSLMMTLCLGSIYEDLKMYQTEFQAINAALQNHNHQQIILDKGMLVAIDELMQ |
| | | SLNHNGETLRQKPPVGEADPYRVKMKLCILLHAFSTRVVTINRVMGYLSSA |
| 219 | P46 | ATGGGCGTGCACGAGTGCCCCGCCTGGCTGTGGCTGCTGCTGAGCCTGCTGAGCCTGCCCCTGGGCCTGCCCGTG |
| | | CTGGGCGCCagagtccaaccaacagaatctattgttagatttcctaatattacaaacttgtgcccttttggtgaagtttttaacgccaccagatttgcatccgtgtatgcttgg |
| | | aacaggaagagaatcagcaactgtgttgctgattattctgtcctatataattccgcatcattttccacttttaagtgttatggagtgtctcctactaaattaaatgatctctgctttactaa |
| | | tgtctatgcagattcatttgtaattagaggtgatgaagtcagacaaatcgctccagggcaaactggaaagattgctgattataattataaattaccagatgattttacaggctgcgt |
| | | tatagcttggaattctaacaatcttgattctaaggttggtggtaattataattacctgtatagattgtttaggaagtctaatctcaaaccttttgagagagatatttcaactgaaatctatc |
| | | aggccggtagcacaccttgtaatggtgttgaaggttttaattgttactttcctttacaatcatatggtttccaacccactaatggtgttggttaccaaccatacagagtagtagtactttc |
| | | ttttgaacttctacatgcaccagcaactgtttgtggacctaaaaagtctactaatttggttaaaaacaaatgtgtcaatttcTAA |
| 220 | p47 | ATGTACCGGATGCAGTTGTTGTCCTGTATAGCTCTTTCCCTTGCATTGGTCACTAATTCTagagtccaaccaacagaatctattgtta |
| | | gatttcctaatattacaaacttgtgcccttttggtgaagtttttaacgccaccagatttgcatccgtgtatgcttggaacaggaagagaatcagcaactgtgttgctgattattctgtcct |
| | | atataattccgcatcattttccacttttaagtgttatggagtgtctcctactaaattaaatgatctctgctttactaatgtctatgcagattcatttgtaattagaggtgatgaagtcagac |
| | | aaatcgctccagggcaaactggaaagattgctgattataattataaattaccagatgattttacaggctgcgttatagcttggaattctaacaatcttgattctaaggttggtggtaatt |
| | | ataattacctgtatagattgtttaggaagtctaatctcaaaccttttgagagagatatttcaactgaaatctatcaggccggtagcacaccttgtaatggtgttgaaggttttaattgtt |
| | | actttcctttacaatcatatggtttccaacccactaatggtgttggttaccaaccatacagagtagtagtactttcttttgaacttctacatgcaccagcaactgtttgtggacctaaaa |
| | | agtctactaatttggttaaaaacaaatgtgtcaatttcTAA |
| 221 | P48 | ATGGGAGTCAAAGTTCTGTTTGCCCTGATCTGCATCGCTGTGGCCGAGGCCAAGagagtccaaccaacagaatctattgttagatttcc |
| | | taatattacaaacttgtgcccttttggtgaagtttttaacgccaccagatttgcatccgtgtatgcttggaacaggaagagaatcagcaactgtgttgctgattattctgtcctatataa |
| | | ttccgcatcattttccacttttaagtgttatggagtgtctcctactaaattaaatgatctctgctttactaatgtctatgcagattcatttgtaattagaggtgatgaagtcagacaaatcg |
| | | ctccagggcaaactggaaagattgctgattataattataaattaccagatgattttacaggctgcgttatagcttggaattctaacaatcttgattctaaggttggtggtaattataatt |
| | | acctgtatagattgtttaggaagtctaatctcaaaccttttgagagagatatttcaactgaaatctatcaggccggtagcacaccttgtaatggtgttgaaggttttaattgttactttcc |
| | | tttacaatcatatggtttccaacccactaatggtgttggttaccaaccatacagagtagtagtactttcttttgaacttctacatgcaccagcaactgtttgtggacctaaaaagtcta |
| | | ctaatttggttaaaaacaaatgtgtcaatttcTAA |
| 222 | P49 | ATGGGCGTGCACGAGTGCCCCGCCTGGCTGTGGCTGCTGCTGAGCCTGCTGAGCCTGCCCCTGGGCCTGCCCGTG |
| | | CTGGGCGCCagagtccaaccaacagaatctattgttagatttcctaatattacaaacttgtgcccttttggtgaagtttttaacgccaccagatttgcatccgtgtatgcttgg |
| | | aacaggaagagaatcagcaactgtgttgctgattattctgtcctatataattccgcatcattttccacttttaagtgttatggagtgtctcctactaaattaaatgatctctgctttactaa |
| | | tgtctatgcagattcatttgtaattagaggtgatgaagtcagacaaatcgctccagggcaaactggaaagattgctgattataattataaattaccagatgattttacaggctgcgt |
| | | tatagcttggaattctaacaatcttgattctaaggttggtggtaattataattacctgtatagattgtttaggaagtctaatctcaaaccttttgagagagatatttcaactgaaatctatc |
| | | aggccggtagcacaccttgtaatggtgttgaaggttttaattgttactttcctttacaatcatatggtttccaacccactaatggtgttggttaccaaccatacagagtagtagtactttc |
| | | ttttgaacttctacatgcaccagcaactgtttgtggacctaaaaagtctactaatttggttaaaaacaaatgtgtcaatttcTAA |
| 223 | P50 | ATGTACCGGATGCAGTTGTTGTCCTGTATAGCTCTTTCCCTTGCATTGGTCACTAATTCTAGGGTTCAGCCTACTGAGT |
| | | CCATCGTGAGATTCCCAAACATCACAAATCTTTGTCCCTTCGGTGAGGTATTCAATGCGACACGATTTGCCTCAGTGTA |
| | | CGCGTGGAATCGGAAGAGGATCTCCAATTGCGTGGCCGACTACTCCGTCTTATACAACTCAGCTAGCTTTTCAACATT |
| | | CAAGTGCTATGGCGTGAGCCCTACCAAGCTCAATGACCTGTGCTTCACTAATGTGTATGCCGACTCTTTTGTCATTCG |
| | | CGGCGACGAGGTCCGACAAATCGCACCGGGCCAAACCGGTAAAATTGCCGACTACAACTACAAGCTGCCTGACGAC |
| | | TTCACCGGCTGCGTAATCGCCTGGAACAGCAATAACCTGGATAGCAAAGTGGGCGGAAACTACAACTACCTGTACCG |
| | | GCTCTTTAGAAAGTCCAACCTGAAACCATTCGAGCGCGATATCTCGACCGAAATCTACCAGGCGGGCAGCACCCCCT |
| | | GTAATGGTGTAGAAGGGTTCAATTGTTACTTTCCACTCCAGAGTTATGGGTTCCAGCCGACCAATGGCGTCGGTTATC |
| | | AACCATATCGCGTTGTGGTGTTGTCCTTTGAGCTGCTACACGCCCCAGCTACAGTGTGCGGGCCAAAGAAAAGCACA |
| | | AATCTTGTCAAGAACAAATGCGTTAACTTTTAA |
| 224 | P52 | ATGTACCGGATGCAGTTGTTGTCCTGTATAGCTCTTTCCCTTGCATTGGTCACTAATTCTGTGAACCTCACCACTCGCA |
| | | CACAGCTACCTCCCGCGTACACTAATTCATTTACCAGGGGCGTCTATTATCCTGATAAGGTGTTCCGGAGTTCAGTGT |
| | | TGCATAGCACTCAAGACCTGTTCCTGCCCTTCTTCTCCAATGTCACTTGGTTTCATGCGATACATGTGTCTGGTACCAA |
| | | CGGAACGAAGAGATTTGATAACCCCGTACTGCCATTCAATGATGGCGTATACTTTGCTTCGACTGAAAAATCCAACAT |
| | | CATCAGGGGCTGGATTTTTGGTACAACGCTTGATTCCAAGACCCAGTCCCTCCTTATTGTGAACAATGCGACCAACGT |
| | | CGTGATAAAGGTCTGTGAGTTTCAGTTCTGCAATGACCCATTCCTTGGAGTGTATTATCACAAGAACAACAAATCGTGG |
| | | ATGGAGTCAGAGTTTAGGGTGTACAGCAGCGCTAACAACTGTACATTTGAGTATGTGAGTCAGCCGTTTCTGATGGAT |
| | | CTTGAGGGCAAACAGGGCAATTTTAAGAATCTCAGAGAATTTGTGTTCAAGAACATTGATGGTTACTTTAAGATCTATA |
| | | GCAAACATACGCCAATCAACTTGGTTCGTGATCTGCCACAGGGATTTAGCGCACTGGAACCTCTCGTTGACTTGCCCA |
| | | TAGGTATTAACATCACCAGGTTCCAGACGCTCTTGGCATTACACCGTAGTTATCTGACCCCCGGGGACTCCAGTTCCG |
| | | GATGGACTGCAGGAGCCGCTGCCTACTATGTGGGTTACCTCCAGCCCAGGACCTTTCTTTTGAAATATAACGAGAAC |
| | | GGCACAATCACTGATGCTGTGGACTGCGCATTGGATCCTTTGTCAGAGACTAAGTGCACTCTGAAGTCATTCACAGTC |
| | | GAGAAAGGCATTTACCAGACGTCTAACTTCAGGGTTCAGCCTACTGAGTCCATCGTGAGATTCCCAAACATCACAAAT |
| | | CTTTGTCCCTTCGGTGAGGTATTCAATGCGACACGATTTGCCTCAGTGTACGCGTGGAATCGGAAGAGGATCTCCAAT |
| | | TGCGTGGCCGACTACTCCGTCTTATACAACTCAGCTAGCTTTTCAACATTCAAGTGCTATGGCGTGAGCCCTACCAAG |
| | | CTCAATGACCTGTGCTTCACTAATGTGTATGCCGACTCTTTTGTCATTCGCGGCGACGAGGTCCGACAAATCGCACCG |
| | | GGCCAAACCGGTAAAATTGCCGACTACAACTACAAGCTGCCTGACGACTTCACCGGCTGCGTAATCGCCTGGAACAG |
| | | CAATAACCTGGATAGCAAAGTGGGCGGAAACTACAACTACCTGTACCGGCTCTTTAGAAAGTCCAACCTGAAACCATT |
| | | CGAGCGCGATATCTCGACCGAAATCTACCAGGGGGGCAGCACCCCCTGTAATGGTGTAGAAGGGTTCAATTGTTACT |
| | | TTCCACTCCAGAGTTATGGGTTCCAGCCGACCAATGGCGTCGGTTATCAACCATATCGCGTTGTGGTGTTGTCCTTTG |
| | | AGCTGCTACACGCCCCAGCTACAGTGTGCGGGCCAAAGAAAAGCACAAATCTTGTCAAGAACAAATGCGTTAACTTTA |
| | | ATTTTAATGGACTCACAGGTACAGGAGTCCTGACCGAATCTAATAAGAAGTTCCTGCCCTTTCAACAGTTCGGACGAG |
| | | ACATTGCCGACACCACCGATGCCGTTCGGGACCCACAGACCTTAGAAATTCTGGATATTACTCCATGTAGTTTTGGGG |
| | | GAGTGTCTGTCATCACCCCTGGCACTAATACATCTAACCAGGTTGCAGTCCTCTACCAGGATGTGAACTGTACCGAAG |
| | | TGCCGGTCGCTATTCACGCAGACCAGCTCACTCCTACCTGGCGGGTGTACTCCACAGGCTCTAACGTGTTTCAGACA |
| | | CGTGCAGGGTGCCTAATCGGCGCAGAGCATGTAAATAACTCCTATGAGTGTGATATCCCCATCGGAGCCGGGATCTG |
| | | CGCTTCCTACCAGACACAAACGAATAGTCCCGGATCTGCCTCAAGCGTGGCATCTCAATCCATTATAGCATATACGAT |
| | | GTCCCTTGGAGCTGAAAACAGCGTTGCGTATTCAAACAATAGTATCGCTATTCCAACCAATTTTACAATTAGCGTGACC |
| | | ACAGAAATACTCCCTGTGAGCATGACCAAGACCAGTGTAGACTGTACTATGTACATCTGCGGCGACAGTACTGAGTGT |
| | | AGCAATCTGCTGCTACAGTATGGGTCCTTCTGTACTCAGCTTAATCGGGCTCTCACCGGAATCGCTGTAGAGCAAGAT |
| | | AAAAACACACAAGAAGTGTTTGCTCAAGTGAAGCAGATCTATAAGACACCTCCCATCAAGGATTTCGGTGGGTTCAAC |
| | | TTTAGCCAGATTCTGCCCGATCCGTCTAAACCGTCCAAGCGAAGTTTCATCGAAGACCTGCTTTTCAATAAGGTCACG |
| | | CTGGCAGATGCTGGATTTATCAAACAGTACGGCGACTGTCTGGGCGATATCGCCGCAAGAGACTTGATATGCGCCCA |
| | | AAAGTTTAATGGGTTAACCGTCCTTCCACCGCTCCTGACAGACGAGATGATCGCCCAGTATACAAGTGCCTTATTAGC |
| | | TGGGACCATTACTAGTGGATGGACATTTGGCGCCGGGGCTGCTCTACAGATACCCTTCGCCATGCAGATGGCTTACC |
| | | GCTTCAACGGAATCGGAGTTACCCAGAACGTACTGTACGAAAATCAGAAACTCATAGCTAATCAATTTAACTCTGCCAT |
| | | CGGGAAGATTCAGGATTCCCTGTCGTCTACAGCGTCCGCCTTGGGGAAACTGCAAGATGTAGTGAACCAGAACGCCC |
| | | AGGCCTTAAATACTCTGGTCAAGCAGTTATCTTCAAATTTCGGAGCAATTAGCTCTGTGTTGAACGATATTCTTTCCAG |
| | | GCTGGACCCTCCAGAAGCCGAAGTGCAAATAGACCGGCTCATCACGGGGCGCTTGCAAAGCCTGCAAACCTATGTCA |
| | | CCCAGCAACTGATTCGAGCAGCCGAGATCCGGGCCAGTGCTAATCTGGCCGCCACAAAAATGAGCGAGTGCGTCCT |
| | | CGGGCAGAGCAAACGCGTAGACTTCTGCGGTAAAGGCTATCACCTGATGAGCTTCCCTCAGAGCGCACCCCACGGG |
| | | GTGGTCTTCCTCCACGTTACCTACGTCCCTGCGCAGGAGAAGAACTTCACTACGGCCCCTGCAATTTGCCACGATGG |
| | | CAAGGCCCACTTTCCCAGGGAGGGGGTCTTCGTTTCCAACGGGACTCATTGGTTCGTGACTCAGAGAAATTTTTATGA |
| | | ACCTCAGATCATTACCACTGATAATACATTCGTGTCTGGCAACTGTGATGTGGTTATTGGGATAGTTAATAATACGGTA |
| | | TACGACCCACTCCAGCCCGAGCTGGACTCCTTCAAAGAGGAGCTGGACAAGTACTTTAAAAATCACACCTCACCTGAT |
| | | GTGGACCTAGGTGACATATCTGGCATAAATGCTAGCGTGGTTAACATTCAGAAGGAAATCGACAGACTCAACGAGGT |
| | | GGCCAAAAATCTGAACGAGAGTCTGATCGACCTGCAGGAGTTGGGAAAATATGAACAGTACATCAAATGGCCATGGT |
| | | ACATCTGGCTGGGCTTCATAGCAGGCCTGATCGCCATCGTCATGGTGACTATTATGCTGTGCTGCATGACATCCTGTT |
| | | GTAGCTGTTTGAAGGGGTGTTGCTCCTGCGGCTCATGCTGCAAATTCGACGAGGACGATTCAGAACCTGTGCTGAAG |
| | | GGAGTGAAGCTGCATTACACATAA |
| 225 | P54 | ATGGGAGTCAAAGTTCTGTTTGCCCTGATCTGCATCGCTGTGGCCGAGGCCAAGgttaatcttacaaccagaactcaattaccccctgc |
| | | atacactaattctttcacacgtggtgtttattaccctgacaaagttttcagatcctcagttttacattcaactcaggacttgttcttacctttcttttccaatgttacttggttccatgcta |
| | | tacatgtctctgggaccaatggtactaagaggtttgataaccctgtcctaccatttaatgatggtgtttattttgcttccactgagaagtctaacataataagaggctggatttttggtact |
| | | actttagattcgaagacccagtccctacttattgttaataacgctactaatgttgttattaaagtctgtgaatttcaattttgtaatgatccatttttgggtgtttattaccacaaaaacaa |
| | | caaaagttggatggaaagtgagttcagagtttattctagtgcgaataattgcacttttgaatatgtctctcagccttttcttatggaccttgaaggaaaacagggtaatttcaaaaatctta |
| | | gggaatttgtgtttaagaatattgatggttattttaaaatatattctaagcacacgcctattaatttagtgcgtgatctccctcagggtttttcggctttagaaccattggtagatttgcca |
| | | ataggtattaacatcactaggtttcaaactttacttgctttacatagaagttatttgactcctggtgattcttcttcaggttggacagctggtgctgcagcttattatgtgggttatcttca |
| | | acctaggacttttctattaaaatataatgaaaatggaaccattacagatgctgtagactgtgcacttgaccctctctcagaaacaaagtgtacgttgaaatccttcactgtagaaaa |
| | | aggaatctatcaaacttctaactttagagtccaaccaacagaatctattgttagatttcctaatattacaaacttgtgcccttttggtgaagtttttaacgccaccagatttgcatccgt |
| | | gtatgcttggaacaggaagagaatcagcaactgtgttgctgattattctgtcctatataattccgcatcattttccacttttaagtgttatggagtgtctcctactaaattaaatgatctct |
| | | gctttactaatgtctatgcagattcatttgtaattagaggtgatgaagtcagacaaatcgctccagggcaaactggaaagattgctgattataattataaattaccagatgattttac |
| | | aggctgcgttatagcttggaattctaacaatcttgattctaaggttggtggtaattataattacctgtatagattgtttaggaagtctaatctcaaaccttttgagagagatatttcaact |
| | | gaaatctatcaggccggtagcacaccttgtaatggtgttgaaggttttaattgttactttcctttacaatcatatggtttccaacccactaatggtgttggttaccaaccatacagagta |
| | | gtagtactttcttttgaacttctacatgcaccagcaactgtttgtggacctaaaaagtctactaatttggttaaaaacaaatgtgtcaatttcaacttcaatggtttaacaggcacagg |
| | | tgttcttactgagtctaacaaaaagtttctgcctttccaacaatttggcagagacattgctgacactactgatgctgtccgtgatccacagacacttgagattcttgacattacaccat |
| | | gttcttttggtggtgtcagtgttataacaccaggaacaaatacttctaaccaggttgctgttctttatcaggatgttaactgcacagaagtccctgttgctattcatgcagatcaactta |
| | | ctcctacttggcgtgtttattctacaggttctaatgtttttcaaacacgtgcaggctgtttaataggggctgaacatgtcaacaactcatatgagtgtgacatacccattggtgcaggt |
| | | atatgcgctagttatcagactcagactaattctcctggcagcgccagcagtgtagctagtcaatccatcattgcctacactatgtcacttggtgcagaaaattcagttgcttactcta |
| | | ataactctattgccatacccacaaattttactattagtgttaccacagaaattctaccagtgtctatgaccaagacatcagtagattgtacaatgtacatttgtggtgattcaactgaa |
| | | tgcagcaatcttttgttgcaatatggcagtttttgtacacaattaaaccgtgctttaactggaatagctgttgaacaagacaaaaacacccaagaagtttttgcacaagtcaaaca |
| | | aatttacaaaacaccaccaattaaagattttggtggttttaatttttcacaaatattaccagatccatcaaaaccaagcaagaggtcatttattgaagatctacttttcaacaaagtg |
| | | acacttgcagatgctggcttcatcaaacaatatggtgattgccttggtgatattgctgctagggacctcatttgtgcacaaaagtttaacggccttactgttttgccacctttgctcac |
| | | agatgaaatgattgctcaatacacttctgcactgttagcgggtacaatcacttctggttggacctttggtgcaggtgctgcattacaaataccatttgctatgcaaatggcttatagg |
| | | tttaatggtattggagttacacagaatgttctctatgagaaccaaaaattgattgccaaccaatttaatagtgctattggcaaaattcaagactcactttcttccacagcaagtgca |
| | | cttggaaaacttcaagatgtggtcaaccaaaatgcacaagctttaaacacgcttgttaaacaacttagctccaattttggtgcaatttcaagtgttttaaatgatatcctttcacgtctt |
| | | gaccctcccgaggctgaagtgcaaattgataggttgatcacaggcagacttcaaagtttgcagacatatgtgactcaacaattaattagagctgcagaaatcagagcttctgct |
| | | aatcttgctgctactaaaatgtcagagtgtgtacttggacaatcaaaaagagttgatttttgtggaaagggctatcatcttatgtccttccctcagtcagcacctcatggtgtagtcttc |
| | | ttgcatgtgacttatgtccctgcacaagaaaagaacttcacaactgctcctgccatttgtcatgatggaaaagcacactttcctcgtgaaggtgtctttgtttcaaatggcacacact |
| | | ggtttgtaacacaaaggaatttttatgaaccacaaatcattactacagacaacacatttgtgtctggtaactgtgatgttgtaataggaattgtcaacaacacagtttatgatcctttg |
| | | caacctgaattagactcattcaaggaggagttagataaatattttaagaatcatacatcaccagatgttgatttaggtgacatctctggcattaatgcttcagttgtaaacattcaaa |
| | | aagaaattgaccgcctcaatgaggttgccaagaatttaaatgaatctctcatcgatctccaagaacttggaaagtatgagcagtatataaaatggccatggtacatttggctag |
| | | gttttatagctggcttgattgccatagtaatggtgacaattatgctttgctgtatgaccagttgctgtagttgtctcaagggctgttgttcttgtggatcctgctgcaaatttgatgaagac |
| | | gactctgagccagtgctcaaaggagtcaaattacattacaca |
| 226 | P56 | atgtttgtttttcttgttttattgccactagtctctagtcagtgtagagtccaaccaacagaatctattgttagatttcctaatattacaaacttgtgcccttttggtgaagtttttaacgc |
| | | caccagatttgcatccgtgtatgcttggaacaggaagagaatcagcaactgtgttgctgattattctgtcctatataattccgcatcattttccacttttaagtgttatggagtgtctccta |
| | | ctaaattaaatgatctctgctttactaatgtctatgcagattcatttgtaattagaggtgatgaagtcagacaaatcgctccagggcaaactggaaagattgctgattataattataaatt |
| | | accagatgattttacaggctgcgttatagcttggaattctaacaatcttgattctaaggttggtggtaattataattacctgtatagattgtttaggaagtctaatctcaaaccttttgag |
| | | agagatatttcaactgaaatctatcaggccggtagcacaccttgtaatggtgttgaaggttttaattgttactttcctttacaatcatatggtttccaacccactaatggtgttggttacc |
| | | aaccatacagagtagtagtactttcttttgaacttctacatgcaccagcaactgtttgtggacctaaaaagtctactaatttggttaaaaacaaatgtgtcaatttcGGGTATAT |
| | | CCCTGAAGCCCCCAGGGACGGCCAGGCTTACGTCAGAAAGGATGGAGAGTGGGTGCTCTTGAGCACCTTCCTGTAA |
| 227 | P59 | atgtttgtttttcttgttttattgccactagtctctagtcagtgtgttaatcttacaaccagaactcaattaccccctgcatacactaattctttcacacgtggtgtttattaccctgaca |
| | | aagttttcagatcctcagttttacattcaactcaggatttgttcttacctttcttttccaatgttacttggttccatgctatacatgtctctgggaccaatggtactaagaggtttgataac |
| | | cctgtcctaccatttaatgatggtgtttattttgcttccactgagaagtctaacataataagaggctggatctttggtactactttagattcgaagacccagtccctacttattgttaataa |
| | | cgctactaatgttgttattaaagtctgtgaatttcaattttgtaatgatccatttttgggtgtttattaccacaaaaacaacaaaagttggatggaaagtgagttcagagtttattctagtg |
| | | cgaataattgcacttttgaatatgtctctcagccttttcttatggaccttgaaggaaaacagggtaatttcaaaaatcttagggaatttgtgtttaagaatattgatggttattttaaaata |
| | | tattctaagcacacgcctattaatttagtgcgtgatctccctcagggtttttcggctttagaaccattggtagatttgccaataggtattaacatcactaggtttcaaactttacttgcttt |
| | | acatagaagttatttgactcctggtgattcttcttcaggttggacagctggtgctgcagcttattatgtgggttatcttcaacctaggacttttctattaaaatataatgaaaatggaacca |
| | | ttacagatgctgtagactgtgcacttgaccctctctcagaaacaaagtgtacgttgaaatccttcactgtagaaaaaggaatctatcaaacttctaactttagagtccaaccaacaga |
| | | atctattgttagatttcctaatattacaaacttgtgcccttttggtgaagtttttaacgccaccagatttgcatccgtgtatgcttggaacaggaagagaatcagcaactgtgttgctga |
| | | ttattctgtcctatataattccgcatcattttccacttttaagtgttatggagtgtctcctactaaattaaatgatctctgctttactaatgtctatgcagattcatttgtaattagaggtg |
| | | atgaagtcagacaaatcgctccagggcaaactggaaagattgctgattataattataaattaccagatgattttacaggctgcgttatagcttggaattctaacaatcttgattctaaggtt |
| | | ggtggtaattataattacctgtatagattgtttaggaagtctaatctcaaaccttttgagagagatatttcaactgaaatctatcaggccggtagcacaccttgtaatggtgttgaag |
| | | gttttaattgttactttcctttacaatcatatggtttccaacccactaatggtgttggttaccaaccatacagagtagtagtactttcttttgaacttctacatgcaccagcaactgtttgt |
| | | ggacctaaaaagtctactaatttggttaaaaacaaatgtgtcaatttcaacttcaatggtttaacaggcacaggtgttcttactgagtctaacaaaaagtttctgcctttccaacaattt |
| | | ggcagagacattgctgacactactgatgctgtccgtgatccacagacacttgagattcttgacattacaccatgttcttttggtggtgtcagtgttataacaccaggaacaaatact |
| | | tctaaccaggttgctgttctttatcaggatgttaactgcacagaagtccctgttgctattcatgcagatcaacttactcctacttggcgtgtttattctacaggttctaatgtttttcaaac |
| | | acgtgcaggctgtttaataggggctgaacatgtcaacaactcatatgagtgtgacatacccattggtgcaggtatatgcgctagttatcagactcagactaattctcctcggcggg |
| | | cacgtagtgtagctagtcaatccatcattgcctacactatgtcacttggtgcagaaaattcagttgcttactctaataactctattgccatacccacaaattttactattagtgttacca |
| | | cagaaattctaccagtgtctatgaccaagacatcagtagattgtacaatgtacatttgtggtgattcaactgaatgcagcaatcttttgttgcaatatggcagtttttgtacacaatta |
| | | aaccgtgctttaactgggatagctgttgaacaagacaaaaacacccaagaagtttttgcacaagtcaaacaaatttacaaaacaccaccaattaaagattttggtggttttaatt |
| | | tttcacaaatattaccagatccatcaaaaccaagcaagaggtcatttattgaagatctacttttcaacaaagtgacacttgcagatgctggcttcatcaaacaatatggtgattgc |
| | | cttggtgatattgctgctagggacctcatttgtgcacaaaagtttaacggccttactgttttgccacctttgctcacagatgaaatgattgctcaatacacttctgcactgttagcgggt |
| | | acaatcacttctggttggacctttggtgcaggtgctgcattacaaataccatttgctatgcaaatggcttataggtttaatggtattggagttacacagaatgttctctatgagaacca |
| | | aaaattgattgccaaccaatttaatagtgccattggcaaaattcaagactcactttcttccacagcaagtgcacttggaaaacttcaagatgtggtcaaccaaaatgcacaagc |
| | | tttaaacacgcttgttaaacaacttagctccaattttggtgcaatttcaagtgttttaaatgatatcctttcacgtcttgaccctcccgaggctgaagtgcaaattgataggttgatcac |
| | | aggcagacttcaaagtttgcagacatatgtgactcaacaattaattagagctgcagaaatcagagcttctgctaatcttgctgctactaaaatgtcagagtgtgtacttggacaat |
| | | caaaaagagttgatttttgtggaaagggctatcatcttatgtccttccctcagtcagcacctcatggtgtagtcttcttgcatgtgacttatgtccctgcacaagaaaagaacttcac |
| | | aactgctcctgccatttgtcatgatggaaaagcacactttcctcgtgaaggtgtctttgtttcaaatggcacacactggtttgtaacacaaaggaatttttatgaaccacaaatcatt |
| | | actacagacaacacatttgtgtctggtaactgtgatgttgtaataggaattgtcaacaacacagtttatgatcctttgcaacctgaattagactcattcaaggaggagttagataaa |
| | | tattttaagaatcatacatcaccagatgttgatttaggtgacatctctggcattaatgcttcagttgtaaacattcaaaaagaaattgaccgcctcaatgaggttgccaagaatttaa |
| | | atgaatctctcatcgatctccaagaacttggaaagtatgagcagtatataaaatggccatggtacatttggctaggttttatagctggcttgattgccatagtaatggtgacaattat |
| | | gctttgctgtatgaccagttgctgtagttgtctcaagggctgttgttcttgtggatcctgctgcaaatttgatgaagacgactctgagccagtgctcaaaggagtcaaattacattac |
| | | aca |
| 228 | P60 | atgtttgtttttcttgttttattgccactagtctctagtcagtgtgttaatcttacaaccagaactcaattaccccctgcatacactaattctttcacacgtggtgtttattaccctgacaa |
| | | agttttcagatcctcagttttacattcaactcaggatttgttcttacctttcttttccaatgttacttggttccatgctatacatgtctctgggaccaatggtactaagaggtttgataacc |
| | | ctgtcctaccatttaatgatggtgtttattttgcttccactgagaagtctaacataataagaggctggatctttggtactactttagattcgaagacccagtccctacttattgttaataac |
| | | gctactaatgttgttattaaagtctgtgaatttcaattttgtaatgatccatttttgggtgtttattaccacaaaaacaacaaaagttggatggaaagtgagttcagagtttattctagtgc |
| | | gaataattgcacttttgaatatgtctctcagccttttcttatggaccttgaaggaaaacagggtaatttcaaaaatcttagggaatttgtgtttaagaatattgatggttattttaaaatat |
| | | attctaagcacacgcctattaatttagtgcgtgatctccctcagggtttttcggctttagaaccattggtagatttgccaataggtattaacatcactaggtttcaaactttacttgcttta |
| | | catagaagttatttgactcctggtgattcttcttcaggttggacagctggtgctgcagcttattatgtgggttatcttcaacctaggacttttctattaaaatataatgaaaatggaaccat |
| | | tacagatgctgtagactgtgcacttgaccctctctcagaaacaaagtgtacgttgaaatccttcactgtagaaaaaggaatctatcaaacttctaactttagagtccaaccaacaga |
| | | atctattgttagatttcctaatattacaaacttgtgcccttttggtgaagtttttaacgccaccagatttgcatccgtgtatgcttggaacaggaagagaatcagcaactgtgttgctga |
| | | ttattctgtcctatataattccgcatcattttccacttttaagtgttatggagtgtctcctactaaattaaatgatctctgctttactaatgtctatgcagattcatttgtaattagaggtg |
| | | atgaagtcagacaaatcgctccagggcaaactggaaagattgctgattataattataaattaccagatgattttacaggctgcgttatagcttggaattctaacaatcttgattctaaggtt |
| | | ggtggtaattataattacctgtatagattgtttaggaagtctaatctcaaaccttttgagagagatatttcaactgaaatctatcaggccggtagcacaccttgtaatggtgttgaag |
| | | gttttaattgttactttcctttacaatcatatggtttccaacccactaatggtgttggttaccaaccatacagagtagtagtactttcttttgaacttctacatgcaccagcaactgtttgt |
| | | ggacctaaaaagtctactaatttggttaaaaacaaatgtgtcaatttcaacttcaatggtttaacaggcacaggtgttcttactgagtctaacaaaaagtttctgcctttccaacaattt |
| | | ggcagagacattgctgacactactgatgctgtccgtgatccacagacacttgagattcttgacattacaccatgttcttttggtggtgtcagtgttataacaccaggaacaaatact |
| | | tctaaccaggttgctgttctttatcagggtgttaactgcacagaagtccctgttgctattcatgcagatcaacttactcctacttggcgtgtttattctacaggttctaatgtttttcaaac |
| | | acgtgcaggctgtttaataggggctgaacatgtcaacaactcatatgagtgtgacatacccattggtgcaggtatatgcgctagttatcagactcagactaattctcctcggcggg |
| | | cacgtagtgtagctagtcaatccatcattgcctacactatgtcacttggtgcagaaaattcagttgcttactctaataactctattgccatacccacaaattttactattagtgttacca |
| | | cagaaattctaccagtgtctatgaccaagacatcagtagattgtacaatgtacatttgtggtgattcaactgaatgcagcaatcttttgttgcaatatggcagtttttgtacacaatta |
| | | aaccgtgctttaactgggatagctgttgaacaagacaaaaacacccaagaagtttttgcacaagtcaaacaaatttacaaaacaccaccaattaaagattttggtggttttaatt |
| | | tttcacaaatattaccagatccatcaaaaccaagcaagaggtcatttattgaagatctacttttcaacaaagtgacacttgcagatgctggcttcatcaaacaatatggtgattgc |
| | | cttggtgatattgctgctagggacctcatttgtgcacaaaagtttaacggccttactgttttgccacctttgctcacagatgaaatgattgctcaatacacttctgcactgttagcgggt |
| | | acaatcacttctggttggacctttggtgcaggtgctgcattacaaataccatttgctatgcaaatggcttataggtttaatggtattggagttacacagaatgttctctatgagaacca |
| | | aaaattgattgccaaccaatttaatagtgccattggcaaaattcaagactcactttcttccacagcaagtgcacttggaaaacttcaagatgtggtcaaccaaaatgcacaagc |
| | | tttaaacacgcttgttaaacaacttagctccaattttggtgcaatttcaagtgttttaaatgatatcctttcacgtcttgaccctcccgaggctgaagtgcaaattgataggttgatcac |
| | | aggcagacttcaaagtttgcagacatatgtgactcaacaattaattagagctgcagaaatcagagcttctgctaatcttgctgctactaaaatgtcagagtgtgtacttggacaat |
| | | caaaaagagttgatttttgtggaaagggctatcatcttatgtccttccctcagtcagcacctcatggtgtagtcttcttgcatgtgacttatgtccctgcacaagaaaagaacttcac |
| | | aactgctcctgccatttgtcatgatggaaaagcacactttcctcgtgaaggtgtctttgtttcaaatggcacacactggtttgtaacacaaaggaatttttatgaaccacaaatcatt |
| | | actacagacaacacatttgtgtctggtaactgtgatgttgtaataggaattgtcaacaacacagtttatgatcctttgcaacctgaattagactcattcaaggaggagttagataaa |
| | | tattttaagaatcatacatcaccagatgttgatttaggtgacatctctggcattaatgcttcagttgtaaacattcaaaaagaaattgaccgcctcaatgaggttgccaagaatttaa |
| | | atgaatctctcatcgatctccaagaacttggaaagtatgagcagtatataaaatggccatggtacatttggctaggttttatagctggcttgattgccatagtaatggtgacaattat |
| | | gctttgctgtatgaccagttgctgtagttgtctcaagggctgttgttcttgtggatcctgctgcaaatttgatgaagacgactctgagccagtgctcaaaggagtcaaattacattac |
| | | aca |
| 229 | P61 | atgtttgtttttcttgttttattgccactagtctctagtcagtgtgttaatcttacaaccagaactcaattaccccctgcatacactaattctttcacacgtggtgtttattaccctgacaa |
| | | agttttcagatcctcagttttacattcaactcaggatttgttcttacctttcttttccaatgttacttggttccatgctatacatgtctctgggaccaatggtactaagaggtttgataacc |
| | | ctgtcctaccatttaatgatggtgtttattttgcttccactgagaagtctaacataataagaggctggatctttggtactactttagattcgaagacccagtccctacttattgttaataa |
| | | cgctactaatgttgttattaaagtctgtgaatttcaattttgtaatgatccatttttgggtgtttattaccacaaaaacaacaaaagttggatggaaagtgagttcagagtttattctagtg |
| | | cgaataattgcacttttgaatatgtctctcagccttttcttatggaccttgaaggaaaacagggtaatttcaaaaatcttagggaatttgtgtttaagaatattgatggttattttaaaata |
| | | tattctaagcacacgcctattaatttagtgcgtgatctccctcagggtttttcggctttagaaccattggtagatttgccaataggtattaacatcactaggtttcaaactttacttgcttt |
| | | acatagaagttatttgactcctggtgattcttcttcaggttggacagctggtgctgcagcttattatgtgggttatcttcaacctaggacttttctattaaaatataatgaaaatggaacca |
| | | ttacagatgctgtagactgtgcacttgaccctctctcagaaacaaagtgtacgttgaaatccttcactgtagaaaaaggaatctatcaaacttctaactttagagtccaaccaacaga |
| | | atctattgttagatttcctaatattacaaacttgtgcccttttggtgaagtttttaacgccaccagatttgcatccgtgtatgcttggaacaggaagagaatcagcaactgtgttgctga |
| | | ttattctgtcctatataattccgcatcattttccacttttaagtgttatggagtgtctcctactaaattaaatgatctctgctttactaatgtctatgcagattcatttgtaattagaggtg |
| | | atgaagtcagacaaatcgctccagggcaaactggaaagattgctgattataattataaattaccagatgattttacaggctgcgttatagcttggaattctaacaatcttgattctaaggtt |
| | | ggtggtaattataattacctgtatagattgtttaggaagtctaatctcaaaccttttgagagagatatttcaactgaaatctatcaggccggtagcacaccttgtaatggtgttgaag |
| | | gttttaattgttactttcctttacaatcatatggtttccaacccactaatggtgttggttaccaaccatacagagtagtagtactttcttttgaacttctacatgcaccagcaactgtttgt |
| | | ggacctaaaaagtctactaatttggttaaaaacaaatgtgtcaatttcaacttcaatggtttaacaggcacaggtgttcttactgagtctaacaaaaagtttctgcctttccaacaattt |
| | | ggcagagacattgctgacactactgatgctgtccgtgatccacagacacttgagattcttgacattacaccatgttcttttggtggtgtcagtgttataacaccaggaacaaatact |
| | | tctaaccaggttgctgttctttatcaggatgttaactgcacagaagtccctgttgctattcatgcagatcaacttactcctacttggcgtgtttattctacaggttctaatgtttttcaaac |
| | | acgtgcaggctgtttaataggggctgaacatgtcaacaactcatatgagtgtgacatacccattggtgcaggtatatgcgctagttatcagactcagactaattctcctcggcggg |
| | | cacgtagtgtagctagtcaatccatcattgcctacactatgtcacttggtgcagaaaattcagttgcttactctaataactctattgccatacccacaaattttactattagtgttacca |
| | | cagaaattctaccagtgtctatgaccaagacatcagtagattgtacaatgtacatttgtggtgattcaactgaatgcagcaatcttttgttgcaatatggcagtttttgtacacaatta |
| | | aaccgtgctttaactgggatagctgttgaacaagacaaaaacacccaagaagtttttgcacaagtcaaacaaatttacaaaacaccaccaattaaagattttggtggttttaatt |
| | | tttcacaaatattaccagatccatcaaaaccaagcaagaggtcatttattgaagatctacttttcaacaaagtgacacttgcagatgctggcttcatcaaacaatatggtgattgc |
| | | cttggtgatattgctgctagggacctcatttgtgcacaaaagtttaacggccttactgttttgccacctttgctcacagatgaaatgattgctcaatacacttctgcactgttagcgggt |
| | | acaatcacttctggttggacctttggtgcaggtgctgcattacaaataccatttgctatgcaaatggcttataggtttaatggtattggagttacacagaatgttctctatgagaacca |
| | | aaaattgattgccaaccaatttaatagtgccattggcaaaattcaagactcactttcttccacagcaagtgcacttggaaaacttcaagatgtggtcaaccaaaatgcacaagc |
| | | tttaaacacgcttgttaaacaacttagctccaattttggtgcaatttcaagtgttttaaatgatatcctttcacgtcttgaccctcccgaggctgaagtgcaaattgataggttgatcac |
| | | aggcagacttcaaagtttgcagacatatgtgactcaacaattaattagagctgcagaaatcagagcttctgctaatcttgctgctactaaaatgtcagagtgtgtacttggacaat |
| | | caaaaagagttgatttttgtggaaagggctatcatcttatgtccttccctcagtcagcacctcatggtgtagtcttcttgcatgtgacttatgtccctgcacaagaaaagaacttcac |
| | | aactgctcctgccatttgtcatgatggaaaagcacactttcctcgtgaaggtgtctttgtttcaaatggcacacactggtttgtaacacaaaggaatttttatgaaccacaaatcatt |
| | | actacagacaacacatttgtgtctggtaactgtgatgttgtaataggaattgtcaacaacacagtttatgatcctttgcaacctgaattagactcattcaaggaggagttagataaa |
| | | tattttaagaatcatacatcaccagatgttgatttaggtgacatctctggcattaatgcttcagttgtaaacattcaaaaagaaattgaccgcctcaatgaggttgccaagaatttaa |
| | | atgaatctctcatcgatctccaagaacttggaaagtatgagcagtatataaaatggccatggtacatttggctaggttttatagctggcttgattgccatagtaatggtgacaattat |
| | | gctttgctgtatgaccagttgctgtagttgtctcaagggctgttgttcttgtggatcctgctgcaaatttgatgaagacgactctgagccagtgctcaaaggagtcaaattacattac |
| | | aca |
| 230 | P63 | ATGGGCGTGCACGAGTGCCCCGCCTGGCTGTGGCTGCTGCTGAGCCTGCTGAGCCTGCCCCTGGGCCTGCCCGTG |
| | | CTGGGCGCCagagtccaaccaacagaatctattgttagatttcctaatattacaaacttgtgcccttttggtgaagtttttaacgccaccagatttgcatccgtgtatgcttgg |
| | | aacaggaagagaatcagcaactgtgttgctgattattctgtcctatataattccgcatcattttccacttttaagtgttatggagtgtctcctactaaattaaatgatctctgctttactaa |
| | | tgtctatgcagattcatttgtaattagaggtgatgaagtcagacaaatcgctccagggcaaactggaaagattgctgattataattataaattaccagatgattttacaggctgcgt |
| | | tatagcttggaattctaacaatcttgattctaaggttggtggtaattataattacctgtatagattgtttaggaagtctaatctcaaaccttttgagagagatatttcaactgaaatctatc |
| | | aggccggtagcacaccttgtaatggtgttgaaggttttaattgttactttcctttacaatcatatggtttccaacccactaatggtgttggttaccaaccatacagagtagtagtactttc |
| | | ttttgaacttctacatgcaccagcaactgtttgtggacctaaaaagtctactaatttggttaaaaacaaatgtgtcaatttcTAA |
| 231 | P64 | ATGTACCGGATGCAGTTGTTGTCCTGTATAGCTCTTTCCCTTGCATTGGTCACTAATTCTagagtccaaccaacagaatctattgtta |
| | | gatttcctaatattacaaacttgtgcccttttggtgaagtttttaacgccaccagatttgcatccgtgtatgcttggaacaggaagagaatcagcaactgtgttgctgattattctgtcct |
| | | atataattccgcatcattttccacttttaagtgttatggagtgtctcctactaaattaaatgatctctgctttactaatgtctatgcagattcatttgtaattagaggtgatgaagtcagac |
| | | aaatcgctccagggcaaactggaaagattgctgattataattataaattaccagatgattttacaggctgcgttatagcttggaattctaacaatcttgattctaaggttggtggtaatt |
| | | ataattacctgtatagattgtttaggaagtctaatctcaaaccttttgagagagatatttcaactgaaatctatcaggccggtagcacaccttgtaatggtgttgaaggttttaattgtt |
| | | actttcctttacaatcatatggtttccaacccactaatggtgttggttaccaaccatacagagtagtagtactttcttttgaacttctacatgcaccagcaactgtttgtggacctaaaa |
| | | agtctactaatttggttaaaaacaaatgtgtcaatttcTAA |
| 232 | 2A6200 | atgttcgtgttcctggtgctgctccccctggtgagctcccagtgcgtcaactttaccaaccggacccagctgcccagtgcctataccaacagcttcacccgcggcgtctactacc |
| | | ccgacaaggtgttccggagcagcgtgctgcacagcacccaggacctcttcctccccttttttagcaacgtgacctggttccacgccatccacgtgagtggcaccaacggcac |
| | | caagcggttcgacaaccctgtgctccccttcaacgacggcgtgtacttcgctagcacagaaaaaagtaacatcattcggggctggatcttcggcaccacactcgacagtaag |
| | | acccagagcctgctgattgtgaataacgccaccaacgtggtgatcaaggtgtgcgagttccagttctgcaactaccccttcctgggcgtgtactaccacaagaacaacaaga |
| | | gctggatggagagcgagttccgcgtgtatagctctgccaacaactgcacctttgagtacgtgagccagcccttcctgatggacctggagggcaagcagggcaatttcaaaa |
| | | acctcagcgagttcgtgttcaagaacatcgacggctacttcaagatctacagcaagcacacccccatcaacctggtgcgggacctgccccagggctttagcgctctggagc |
| | | ccctggtggacctgcccatcggcatcaacatcacccgcttccaaaccctgttggccctgcaccggagctacctgacccctggcgatagcagtagtggctggacagccggag |
| | | ccgccgcctactacgtgggatacctgcagccccggaccttcctgctgaagtacaacgagaacggcacaatcacagacgctgtggactgcgccctggaccccctgagcga |
| | | aacaaagtgcaccctcaaaagtttcaccgtggagaagggcatctaccagaccagcaacttccgggtgcagcccaccgagagcatcgtgcggttccccaacatcaccaac |
| | | ctgtgccccttcggcgaggtgttcaacgccacccggttcgccagcgtgtacgcctggaaccggaagcggatcagcaactgcgtggccgactacagcgtgctgtataacagt |
| | | gccagcttcagcaccttcaagtgctacggcgtgagccccaccaagctgaacgacctgtgcttcaccaacgtgtacgccgatagctttgtgatccggggcgacgaggtgcgg |
| | | cagatcgcccccggccagacaggcacaattgccgattacaattacaagctgcccgacgatttcaccggctgcgtgatcgcctggaacagcaacaacctggacagcaagg |
| | | tgggcggaaactacaactacctgtaccggctgttccgcaaaagtaacctcaagcctttcgagcgggacatcagcaccgagatctaccaggccggcagtaccccttgcaac |
| | | ggcgtgaagggcttcaactgctacttccccctgcagagctacggcttccagcccacctacggcgtgggctaccagccctaccgggtggtggttctgagcttcgagctgctgca |
| | | cgcccctgccaccgtgtgcggccccaagaaaagcaccaacctggtgaagaacaagtgcgtgaacttcaactttaacggactgacaggcaccggcgtgctgaccgagag |
| | | caacaagaagttcctgcccttccagcaattcggccgggatattgccgacaccaccgacgccgtgcgggacccccagaccctggagatcctggacatcacaccctgtagctt |
| | | cggcggcgtgagcgtgatcacccccggcaccaacaccagcaaccaggtggccgtgctctaccaaggcgtgaactgcaccgaggtgcccgtggccatccacgccgacc |
| | | agctgacccccacctggcgggtgtacagcaccggcagcaacgtgttccagacccgggccggctgcctgatcggcgccgagtacgtgaataacagctacgagtgcgacat |
| | | ccccatcggagccggaatctgcgccagctaccagacccagaccaacagcccccggagagcccggagcgtggccagccagagcattattgcctacaccatgagcctgg |
| | | gcgccgagaacagcgtggcctacagcaacaatagtattgccatccccaccaacttcaccatcagcgtgaccactgaaatcctccccgtgagcatgaccaagaccagcgtg |
| | | gactgcaccatgtacatctgcggcgacagcaccgagtgcagcaacctgctcctgcagtacggcagcttctgcacccagctgaaccgggccctgaccggaattgccgtgga |
| | | gcaggacaagaacacccaggaggtgttcgcccaggtgaagcagatctacaagacacctcctatcaaggacttcgggggttcaacttcagccagatcctgcccgacccc |
| | | agcaagcccagcaagcggagcttcatcgaggacctgttgttcaacaaggtgaccctggccgacgccggcttcatcaagcagtacggcgactgtctgggagatattgccgc |
| | | acgggacctgatctgcgcccagaagttcaacggcctgaccgtgctgcctcctctgctgaccgacgagatgatcgcccagtacacaagcgctctgctcgccggcaccatcac |
| | | cagcggctggaccttcggcgccggcgctgccctgcagatccccttcgccatgcagatggcctaccggttcaacggcatcggcgtgacccagaacgtgctgtacgagaacc |
| | | agaagctgatcgccaaccagttcaacagcgccatcggcaagatccaggacagcctgagctccaccgccagcgccctgggcaagctgcaggacgtggtgaaccagaac |
| | | gcccaggccctgaacaccctggtgaagcagctgagcagcaacttcggcgccatcagctctgtgctgaacgacatcctgagccggctggaccccccagaggccgaggtgc |
| | | aaatcgatcggctcatcaccggccggctgcagagcctgcagacctacgtgacacagcagctgatccgggccgccgagatccgggccagcgccaacctggccgctatca |
| | | agatgagcgagtgcgtgctgggccagagcaagcgggtggacttctgcggcaagggctaccacctgatgagcttcccccagagcgccccccacggcgtggtgttcctgcac |
| | | gtgacctacgtgcccgcccaggagaagaacttcaccaccgcccccgccatctgccacgacggcaaggcccacttcccccgggagggcgtgttcgtgagcaacggcacc |
| | | cactggttcgtgacccagcggaacttctacgagccccagatcataaccaccgacaacaccttcgtgagcggcaactgcgacgtggtcatcggcatcgtgaacaacaccgt |
| | | gtacgaccccctgcagcccgagctggacagcttcaaggaggaactggacaagtacttcaagaaccacaccagccccgacgtggacctgggcgacatcagcggcatca |
| | | acgccagcttcgtgaacatccagaaggagatcgaccggctgaacgaggtggccaagaacctgaacgagagcctgatcgacctgcaggagctgggcaagtacgagca |
| | | gtacatcaagtggccctggtacatctggctgggctttattgccggcctgatcgccatcgtgatggtgaccatcatgctgtgctgtatgacaagctgttgcagttgcctgaagggct |
| | | gctgcagctgcggcagctgctgcaagttcgacgaggacgacagcgagcccgtgctgaagggcgtgaagctgcactacacc |
| 233 | 2A0620 | atgttcgtgttcctggtgctgctccccctggtgagctcccagtgcgtcaacctgaccacacggacccagctgccccccgcctataccaacagcttcacccgcggcgtctactac |
| | | cccgacaaggtgttccggagcagcgtgctgcacagcacccaggacctcttcctccccttttttagcaacgtgacctggttccacgccatccacgtgagtggcaccaacggca |
| | | ccaagcggttcgccaaccctgtgctccccttcaacgacggcgtgtacttcgctagcacagaaaaaagtaacatcattcggggctggatcttcggcaccacactggatagcaa |
| | | aacccagagcctgctgattgtgaataacgccaccaacgtggtgatcaaggtgtgcgagttccagttctgcaacgaccccttcctgggcgtgtactaccacaagaacaacaa |
| | | gagctggatggagagcgagttccgcgtgtatagctctgccaacaactgcaccttcgagtacgtgagccagcccttcctgatggacctggagggcaagcagggcaatttcaa |
| | | aaacctgcgggagttcgtgttcaagaacatcgacggctacttcaagatctacagcaagcacacccccatcaacctggtgcggggcctgccccagggctttagcgctctgga |
| | | gcccctggtggacctgcccatcggcatcaacatcacccgcttccaaaccctgttggccctgcaccggagctacctgacccctggcgatagcagtagtggctggacagccgg |
| | | agccgccgcctactacgtgggatacctgcagccccggaccttcctgctgaagtacaacgagaacggcacaatcacagacgccgtggactgcgccctggaccccctgagc |
| | | gaaacaaagtgcaccctcaaaagtttcaccgtggagaagggcatctaccagaccagcaacttccgggtgcagcccaccgagagcatcgtgcggttccccaacatcacca |
| | | acctgtgccccttcggcgaggtgttcaacgccacccggttcgccagcgtgtacgcctggaaccggaagcggatcagcaactgcgtggccgactacagcgtgctgtataaca |
| | | gtgccagcttcagcaccttcaagtgctacggcgtgagccccaccaagctgaacgacctgtgcttcaccaacgtgtacgccgacagcttcgtgatccggggcgacgaggtgc |
| | | ggcagatcgcccccggccagaccggcaatattgctgactataactataagctgcccgacgatttcaccggctgcgtgatcgcctggaacagcaacaacctggacagcaag |
| | | gtgggcggaaactacaactacctgtaccggctgttccgcaaaagtaacctcaagcctttcgagcgggacatcagcaccgagatctaccaggccggcagtaccccttgcaa |
| | | cggcgtgaagggcttcaactgctacttccccctgcagagctacggcttccagcccacctacggcgtgggctaccagccctaccgggtggtggttctgagcttcgagctgctgc |
| | | acgcccctgccaccgtgtgcggccccaagaaaagcaccaacctggtgaagaacaagtgcgtgaacttcaactttaacggactgacaggcaccggcgtgctgaccgaga |
| | | gcaacaagaagttcctgcccttccagcaattcggccgggatattgccgacaccactgacgccgtgcgggacccccagaccctggagatcctggacatcacaccctgtagct |
| | | tcggcggcgtgagcgtgatcacccccggcaccaacaccagcaaccaggtggccgtgctctaccaaggcgtgaactgcaccgaggtgcccgtggccatccacgccgacc |
| | | agctgacccccacctggcgggtgtacagcaccggcagcaacgtgttccagacccgggccggctgcctgatcggcgccgagcacgtgaataacagctacgagtgcgaca |
| | | tccccatcggagccggaatctgcgccagctaccagacccagaccaacagcccccggagagcccggagcgtggccagccagagcattattgcctacaccatgagcctgg |
| | | gcgtggagaacagcgtggcctacagcaacaatagtattgccatccccaccaacttcaccatcagcgtgaccaccgaaatcctccccgtgagcatgaccaagaccagcgt |
| | | ggactgcaccatgtacatctgcggcgacagcaccgagtgcagcaacctgctcctgcagtacggcagcttctgcacccagctgaaccgggccctgaccggaattgccgtgg |
| | | agcaggacaagaacacccaggaggtgttcgcccaggtgaagcagattacaagacacctcccatcaaggacttcgggggttcaacttcagccagatcctgcccgaccc |
| | | cagcaagcccagcaagcggagcttcatcgaggacctgttgttcaacaaggtgaccctggccgacgccggcttcatcaagcagtacggcgactgtctgggagatattgccg |
| | | cacgggacctgatctgcgcccagaagttcaacggcctgaccgtgctgcccccactgctgaccgacgagatgatcgcccagtacacaagcgctctgctcgccggcaccatc |
| | | accagcggctggaccttcggcgccggcgctgccctgcagatccccttcgccatgcagatggcctaccggttcaacggcatcggcgtgacccagaacgtgctgtacgagaa |
| | | ccagaagctgatcgccaaccagttcaacagcgccatcggcaagatccaggacagcctgagctccaccgccagcgccctgggcaagctgcaggacgtggtgaaccaga |
| | | acgcccaggccctgaacaccctggtgaagcagctgagcagcaacttcggcgccatcagctctgtgctgaacgacatcctgagccggctggatcctcctgaggccgaggtg |
| | | caaatcgatcggctcatcaccggccggctgcagagcctgcagacctacgtgacacagcagctgatccgggccgccgagatccgggccagcgccaacctggccgctacc |
| | | aagatgagcgagtgcgtgctgggccagagcaagcgggtggacttctgcggcaagggctaccacctgatgagcttcccccagagcgccccccacggcgtggtgttcctgca |
| | | cgtgacctacgtgcccgcccaggagaagaacttcaccaccgcccccgccatctgccacgacggcaaggcccacttcccccgggagggcgtgttcgtgagcaacggcac |
| | | ccactggttcgtgacccagcggaacttctacgagccccagatcataaccacagacaacaccttcgtgagcggcaactgcgacgtggtcatcggcatcgtgaacaacaccg |
| | | tgtacgaccccctgcagcccgagctggacagcttcaaggaggaactggacaagtacttcaagaaccacaccagccccgacgtggacctgggcgacatcagcggcatca |
| | | acgccagcgtggtgaacatccagaaggagatcgaccggctgaacgaggtggccaagaacctgaacgagagcctgatcgacctgcaggagctgggcaagtacgagca |
| | | gtacatcaagtggccctggtacatctggctgggctttattgccggcctgatcgccatcgtgatggtgaccatcatgctgtgctgtatgacaagctgttgcagttgcctgaagggct |
| | | gctgcagctgcggcagctgctgcaagttcgacgaggacgacagcgagcccgtgctgaagggcgtgaagctgcactacacc |
| 234 | 2A3430 | atgttcgtgttcctggtgctgctccccctggtgagctcccagtgcgtcaacctgaccacacggacccagctgccccccgcctataccaacagcttcacccggggcgtgtactat |
| | | cccgacaaggtgttccggagcagcgtgctgcacagcacccaggacctcttcctccccttttttagcaacgtgacctggttccacgctatcagtggcaccaacggcaccaagc |
| | | ggttcgacaaccctgtgctccccttcaacgacggcgtgtacttcgctagcacagaaaaaagtaacatcattcggggctggatcttcggcaccacactggatagcaaaaccca |
| | | gagcctgctgattgtgaataacgccaccaacgtggtgatcaaggtgtgcgagttccagttctgcaacgaccccttcctgggcgtgtaccacaagaacaacaagagctggatg |
| | | gagagcgagttccgcgtgtatagctctgccaacaactgcaccttcgagtacgtgagccagcccttcctgatggacctggagggcaagcagggcaatttcaaaaacctgcgg |
| | | gagttcgtgttcaagaacatcgacggctacttcaagatctacagcaagcacacccccatcaacctggtgcgggacctgccccagggctttagcgctctggagcccctggtgg |
| | | acctgcccatcggcatcaacatcacccgcttccaaaccctgttggccctgcaccggagctacctgacccctggcgatagcagtagtggctggacagccggagccgccgcct |
| | | actacgtgggatacctgcagccccggaccttcctgctgaagtacaacgagaacggcacaatcacagacgccgtggactgcgccctggaccccctgagcgaaacaaagt |
| | | gcaccctcaaaagtttcaccgtggagaagggcatctaccagaccagcaacttccgggtgcagcccaccgagagcatcgtgcggttccccaacatcaccaacctgtgcccc |
| | | ttcggcgaggtgttcaacgccacccggttcgccagcgtgtacgcctggaaccggaagcggatcagcaactgcgtggccgactacagcgtgctgtataacagtgccagcttc |
| | | agcaccttcaagtgctacggcgtgagccccaccaagctgaacgacctgtgcttcaccaacgtgtacgccgacagcttcgtgatccggggcgacgaggtgcggcagatcgc |
| | | ccccggccagaccggcaagatcgccgattacaattacaagctgcccgacgatttcaccggctgcgtgatcgcctggaacagcaacaacctggacagcaaggtgggcgg |
| | | aaactacaactacctgtaccggctgttccgcaaaagtaacctgaagcccttcgagcgggacatcagcaccgagatctaccaggccggcagtaccccttgcaacggcgtgg |
| | | agggcttcaactgctacttccccctgcagagctacggcttccagcccacctacggcgtgggctaccagccctaccgggtggtggttctgagcttcgagctgctgcacgcccct |
| | | gccaccgtgtgcggccccaagaaaagcaccaacctggtgaagaacaagtgcgtgaacttcaactttaacggactgacaggcaccggcgtgctgaccgagagcaacaa |
| | | gaagttcctgcccttccagcaattcggccgggacatcgacgacaccactgacgccgtggggacccccagaccctggagatcctggacatcacaccctgtagcttcggcg |
| | | gcgtgagcgtgatcacccccggcaccaacaccagcaaccaggtggccgtgctctaccaaggcgtgaactgcaccgaggtgcccgtggccatccacgccgaccagctga |
| | | cccccacctggcgggtgtacagcaccggcagcaacgtgttccagacccgggccggctgcctgatcggcgccgagcacgtgaataacagctacgagtgcgacatccccat |
| | | cggagccggaatctgcgccagctaccagacccagaccaacagccaccggagagcccggagcgtggccagccagagcattattgcctacaccatgagcctgggcgccg |
| | | agaacagcgtggcctacagcaacaatagtattgccatccccaccaacttcaccatcagcgtgaccaccgaaatcctccccgtgagcatgaccaagaccagcgtggactgc |
| | | accatgtacatctgcggcgacagcaccgagtgcagcaacctgctcctgcagtacggcagcttctgcatccagctgaaccgggccctgaccggaattgccgtggagcagga |
| | | caagaacacccaggaggtgttcgcccaggtgaagcagatctacaagacacctcccatcaaggacttcgggggttcaacttcagccagatcctgcccgaccccagcaag |
| | | cccagcaagcggagcttcatcgaggacctgttgttcaacaaggtgaccctggccgacgccggcttcatcaagcagtacggcgactgtctgggagatattgccgcacggga |
| | | cctgatctgcgcccagaagttcaacggcctgaccgtgctgcccccactgctgaccgacgagatgatcgcccagtacacaagcgctctgctcgccggcaccatcaccagcg |
| | | gctggaccttcggcgccggcgctgccctgcagatccccttcgccatgcagatggcctaccggttcaacggcatcggcgtgacccagaacgtgctgtacgagaaccagaag |
| | | ctgatcgccaaccagttcaacagcgccatcggcaagatccaggacagcctgagctccaccgccagcgccctgggcaagctgcaggacgtggtgaaccagaacgccca |
| | | ggccctgaacaccctggtgaagcagctgagcagcaacttcggcgccatcagctctgtgctgaacgacatcctggcccggctggatcctcctgaggccgaggtgcaaatcg |
| | | atcggctcatcaccggccggctgcagagcctgcagacctacgtgacacagcagctgatccgggccgccgagatccgggccagcgccaacctggccgctaccaagatga |
| | | gcgagtgcgtgctgggccagagcaagcgggtggacttctgcggcaagggctaccacctgatgagcttcccccagagcgccccccacggcgtggtgttcctgcacgtgacc |
| | | tacgtgcccgcccaggagaagaacttcaccaccgcccccgccatctgccacgacggcaaggcccacttcccccgggagggcgtgttcgtgagcaacggcacccactggt |
| | | tcgtgacccagcggaacttctacgagccccagatcataaccacacacaacaccttcgtgagcggcaactgcgacgtggtcatcggcatcgtgaacaacaccgtgtacgac |
| | | cccctgcagcccgagctggacagcttcaaggaggaactggacaagtacttcaagaaccacaccagccccgacgtggacctgggcgacatcagcggcatcaacgccag |
| | | cgtggtgaacatccagaaggagatcgaccggctgaacgaggtggccaagaacctgaacgagagcctgatcgacctgcaggagctgggcaagtacgagcagtacatca |
| | | agtggccctggtacatctggctgggctttattgccggcctgatcgccatcgtgatggtgaccatcatgctgtgctgtatgacaagctgttgcagttgcctgaagggctgctgcagct |
| | | gcggcagctgctgcaagttcgacgaggacgacagcgagcccgtgctgaagggcgtgaagctgcactacacc |
| 235 | 2A8210 | atgggcgtcaaggtcctgttcgccctgatctgtattgccgtggccgaggccaagcgggtgcagcccaccgagagcatcgtgcggttccccaacatcaccaacctgtgcccctt |
| | | cggcgaggtgttcaacgccacccggttcgccagcgtgtacgcctggaaccggaagcggatcagcaactgcgtggccgactacagcgtgctgtacaacagcgccagcttc |
| | | agcaccttcaagtgctacggcgtgagccccaccaagctgaacgacctgtgcttcaccaacgtgtacgccgacagcttcgtgatccggggcgacgaggtgcggcagatcgc |
| | | ccccggccagacaggcaacattgctgattacaattacaagctgcccgacgatttcaccggctgcgtgatcgcctggaacagcaacaatctggacagcaaggtgggcgga |
| | | aactacaactacctgtaccggctgttccgcaaaagtaacctcaagcctttcgagcgggacatcagcaccgagatctaccaggccggcagcaccccctgcaacggcgtga |
| | | agggcttcaactgctacttccccctgcagagctacggcttccagcccacctacggcgtgggctaccagccctaccgggtggtcgtgctgagcttcgagctgctccacgccccc |
| | | gccaccgtgtgcggccccaagaaaagcaccaacctggtgaagaacaagtgcgtgaacttc |
| 236 | 2A9200 | atgttcgtgttcctggtgctgctccccctggtgagctcccagtgcgtcaactttaccacccggacccagctgccccccgcctataccaacagcttcacccgcggcgtctactacc |
| | | ccgacaaggtgttccggagcagcgtgctgcacagcacccaggacctcttcctccccttttttagcaacgtgacctggttccacgccatccacgtgagtggcaccaacggcac |
| | | caagcggttcgccaaccctgtgctccccttcaacgacggcgtgtacttcgctagcacagaaaaaagtaacatcattcggggctggatcttcggcaccacactggatagcaaa |
| | | acccagagcctgctgattgtgaataacgccaccaacgtggtgatcaaggtgtgcgagttccagttctgcaacgaccccttcctgggcgtgtactaccacaagaacaacaag |
| | | agctggatggagagcgagttccgcgtgtatagctctgccaacaactgcaccttcgagtacgtgagccagcccttcctgatggacctggagggcaagcagggcaatttcaaa |
| | | aacctgcgggagttcgtgttcaagaacatcgacggctacttcaagatctacagcaagcacacccccatcaacctggtgcggggcctgccccagggctttagcgctctggag |
| | | cccctggtggacctgcccatcggcatcaacatcacccgcttccaaaccctgcacatcagctacctgacccctggcgatagcagtagtggctggacagccggagccgccgc |
| | | ctactacgtgggatacctgcagccccggaccttcctgttgaagtacaacgagaacggcacaatcacagacgccgtggactgcgccctggaccccctgagcgaaacaaag |
| | | tgcaccctcaaaagtttcaccgtggagaagggcatctaccagaccagcaacttccgggtgcagcccaccgagagcatcgtgcggttccccaacatcaccaacctgtgccc |
| | | cttcggcgaggtgttcaacgccacccggttcgccagcgtgtacgcctggaaccggaagcggatcagcaactgcgtggccgactacagcgtgctgtataacagtgccagctt |
| | | cagcaccttcaagtgctacggcgtgagccccaccaagctgaacgacctgtgcttcaccaacgtgtacgccgacagcttcgtgatccggggcgacgaggtgcggcagatcg |
| | | cccccggccagaccggcaatattgctgactataactataagctgcccgacgatttcaccggctgcgtgatcgcctggaacagcaacaacctggacagcaaggtgggcgga |
| | | aactacaactacctgtaccggctgttccgcaaaagtaacctcaagcctttcgagcgggacatcagcaccgagatctaccaggccggcagtaccccttgcaacggcgtgaa |
| | | gggcttcaactgctacttccccctgcagagctacggcttccagcccacctacggcgtgggctaccagccctaccgggtggtggttctgagcttcgagctgctgcacgcccctg |
| | | ccaccgtgtgcggccccaagaaaagcaccaacctggtgaagaacaagtgcgtgaacttcaactttaacggactgacaggcaccggcgtgctgaccgagagcaacaag |
| | | aagttcctgcccttccagcaattcggccgggatattgccgacaccactgacgccgtgcgggacccccagaccctggagatcctggacatcacaccctgtagcttcggcggc |
| | | gtgagcgtgatcacccccggcaccaacaccagcaaccaggtggccgtgctctaccaaggcgtgaactgcaccgaggtgcccgtggccatccacgccgaccagctgacc |
| | | cccacctggcgggtgtacagcaccggcagcaacgtgttccagacccgggccggctgcctgatcggcgccgagcacgtgaataacagctacgagtgcgacatccccatcg |
| | | gagccggaatctgcgccagctaccagacccagaccaacagcccccggagagcccggagcgtggccagccagagcattattgcctacaccatgagcctgggcgtggag |
| | | aacagcgtggcctacagcaacaatagtattgccatccccaccaacttcaccatcagcgtgaccaccgaaatcctccccgtgagcatgaccaagaccagcgtggactgcac |
| | | catgtacatctgcggcgacagcaccgagtgcagcaacctgctgctccagtacggcagcttctgcacccagctgaaccgggccctgaccggaattgccgtggagcaggac |
| | | aagaacacccaggaggtgttcgcccaggtgaagcagattacaagacacctcccatcaaggacttcgggggttcaacttcagccagatcctgcccgaccccagcaagc |
| | | ccagcaagcggagcttcatcgaggacctgctgttcaacaaggtgaccctggccgacgccggcttcatcaagcagtacggcgactgtctgggagatattgccgcacgggac |
| | | ctgatctgcgcccagaagttcaacggcctgaccgtgctgcccccactgttgaccgacgagatgatcgcccagtacacaagcgctctgctggccggcaccatcaccagcgg |
| | | ctggaccttcggcgccggcgctgccctgcagatccccttcgccatgcagatggcctaccggttcaacggcatcggcgtgacccagaacgtgctgtacgagaaccagaagct |
| | | gatcgccaaccagttcaacagcgccatcggcaagatccaggacagcctgagctccaccgccagcgccctgggcaagctgcaggacgtggtgaaccagaacgcccag |
| | | gccctgaacaccctggtgaagcagctgagcagcaacttcggcgccatcagctctgtgctgaacgacatcctgagccggctggatcctcctgaggccgaggtgcaaatcgat |
| | | cggctcatcaccggccggctgcagagcctgcagacctacgtgacacagcagctgatccgggccgccgagatccgggccagcgccaacctggccgctaccaagatgag |
| | | cgagtgcgtgctgggccagagcaagcgggtggacttctgcggcaagggctaccacctgatgagcttcccccagagcgccccccacggcgtggtgttcctgcacgtgacct |
| | | acgtgcccgcccaggagaagaacttcaccaccgcccccgccatctgccacgacggcaaggcccacttcccccgggagggcgtgttcgtgagcaacggcacccactggtt |
| | | cgtgacccagcggaacttctacgagccccagatcataaccacagacaacaccttcgtgagcggcaactgcgacgtggtcatcggcatcgtgaacaacaccgtgtacgac |
| | | cccctgcagcccgagctggacagcttcaaggaggaactggacaagtacttcaagaaccacaccagccccgacgtggacctgggcgacatcagcggcatcaacgccag |
| | | cgtggtgaacatccagaaggagatcgaccggctgaacgaggtggccaagaacctgaacgagagcctgatcgacctgcaggagctgggcaagtacgagcagtacatca |
| | | agtggccctggtacatctggctgggctttattgccggcctgatcgccatcgtgatggtgaccatcatgctgtgctgtatgacaagctgttgcagttgcctgaagggctgctgcagct |
| | | gcggcagctgctgcaagttcgacgaggacgacagcgagcccgtgctgaagggcgtgaagctgcactacacc |
| 237 | 2A7020 | ATGTTCGTGTTCCTGGTGCTGCTGCCTCTGGTGTCCAGCCAGTGTGTGAACCTGACCACCAGAACACAGCTGCCTCC |
| | | AGCCTACACCAACAGCTTTACCAGAGGCGTGTACTACCCCGACAAGGTGTTCAGATCCAGCGTGCTGCACTCTACCC |
| | | AGGACCTGTTCCTGCCTTTCTTCAGCAACGTGACCTGGTTCCACGCCATCCACGTGTCCGGCACCAATGGCACCAAG |
| | | AGATTCGACAACCCCGTGCTGCCCTTCAACGACGGGGTGTACTTTGCCAGCACCGAGAAGTCCAACATCATCAGAGG |
| | | CTGGATCTTCGGCACCACACTGGACAGCAAGACCCAGAGCCTGCTGATCGTGAACAACGCCACCAACGTGGTCATCA |
| | | AAGTGTGCGAGTTCCAGTTCTGCAACGACCCCTTCCTGGGCGTCTACTACCACAAGAACAACAAGAGCTGGATGGAA |
| | | AGCGAGTTCCGGGTGTACAGCAGCGCCAACAACTGCACCTTCGAGTACGTGTCCCAGCCTTTCCTGATGGACCTGGA |
| | | AGGCAAGCAGGGCAACTTCAAGAACCTGCGCGAGTTCGTGTTTAAGAACATCGACGGCTACTTCAAGATCTACAGCA |
| | | AGCACACCCCTATCAACCTCGTGCGGGATCTGCCTCAGGGCTTCTCTGCTCTGGAACCCCTGGTGGATCTGCCCATC |
| | | GGCATCAACATCACCCGGTTTCAGACACTGCTGGCCCTGCACAGAAGCTACCTGACACCTGGCGATAGCAGCAGCG |
| | | GATGGACAGCTGGTGCCGCCGCTTACTATGTGGGCTACCTGCAGCCTAGAACCTTCCTGCTGAAGTACAACGAGAAC |
| | | GGCACCATCACCGACGCCGTGGATTGTGCTCTGGATCCTCTGAGCGAGACAAAGTGCACCCTGAAGTCCTTCACCGT |
| | | GGAAAAGGGCATCTACCAGACCAGCAACTTCCGGGTGCAGCCCACCGAATCCATCGTGCGGTTCCCCAATATCACCA |
| | | ATCTGTGCCCCTTCGGCGAGGTGTTCAATGCCACCAGATTCGCCTCTGTGTACGCCTGGAACCGGAAGCGGATCAGC |
| | | AATTGCGTGGCCGACTACTCCGTGCTGTACAACTCCGCCAGCTTCAGCACCTTCAAGTGCTACGGCGTGTCCCCTAC |
| | | CAAGCTGAACGACCTGTGCTTCACAAACGTGTACGCCGACAGCTTCGTGATCCGGGGAGATGAAGTGCGGCAGATTG |
| | | CCCCTGGACAGACAGGCAAGATCGCCGACTACAACTACAAGCTGCCCGACGACTTCACCGGCTGTGTGATTGCCTG |
| | | GAACAGCAACAACCTGGACTCCAAAGTCGGCGGCAACTACAATTACCTGTACCGGCTGTTCCGGAAGTCCAATCTGA |
| | | AGCCCTTCGAGCGGGACATCTCCACCGAGATCTATCAGGCCGGCAGCACCCCTTGTAACGGCGTGGAAGGCTTCAA |
| | | CTGCTACTTCCCACTGCAGTCCTACGGCTTTCAGCCCACAAATGGCGTGGGCTATCAGCCCTACAGAGTGGTGGTGC |
| | | TGAGCTTCGAACTGCTGCATGCCCCTGCCACAGTGTGCGGCCCTAAGAAAAGCACCAATCTCGTGAAGAACAAATGC |
| | | GTGAACTTCAACTTCAACGGCCTGACCGGCACCGGCGTGCTGACAGAGAGCAACAAGAAGTTCCTGCCATTCCAGCA |
| | | GTTTGGCCGGGATATCGCCGATACCACAGACGCCGTTAGAGATCCCCAGACACTGGAAATCCTGGACATCACCCCTT |
| | | GCAGCTTCGGCGGAGTGTCTGTGATCACCCCTGGCACCAACACCAGCAATCAGGTGGCAGTGCTGTACCAGGACGT |
| | | GAACTGTACCGAAGTGCCCGTGGCCATTCACGCCGATCAGCTGACACCTACATGGCGGGTGTACTCCACCGGCAGC |
| | | AATGTGTTTCAGACCAGAGCCGGCTGTCTGATCGGAGCCGAGCACGTGAACAATAGCTACGAGTGCGACATCCCCAT |
| | | CGGCGCTGGAATCTGCGCCAGCTACCAGACACAGACAAACAGCCCTCGGAGAGCCAGAAGCGTGGCCAGCCAGAG |
| | | CATCATTGCCTACACAATGTCTCTGGGCGCCGAGAACAGCGTGGCCTACTCCAACAACTCTATCGCTATCCCCACCAA |
| | | CTTCACCATCAGCGTGACCACAGAGATCCTGCCTGTGTCCATGACCAAGACCAGCGTGGACTGCACCATGTACATCT |
| | | GCGGCGATTCCACCGAGTGCTCCAACCTGCTGCTGCAGTACGGCAGCTTCTGCACCCAGCTGAATAGAGCCCTGAC |
| | | AGGGATCGCCGTGGAACAGGACAAGAACACCCAAGAGGTGTTCGCCCAAGTGAAGCAGATCTACAAGACCCCTCCTA |
| | | TCAAGGACTTCGGCGGCTTCAATTTCAGCCAGATTCTGCCCGATCCTAGCAAGCCCAGCAAGCGGAGCTTCATCGAG |
| | | GACCTGCTGTTCAACAAAGTGACACTGGCCGACGCCGGCTTCATCAAGCAGTATGGCGATTGTCTGGGCGACATTGC |
| | | CGCCAGGGATCTGATTTGCGCCCAGAAGTTTAACGGACTGACAGTGCTGCCTCCTCTGCTGACCGATGAGATGATCG |
| | | CCCAGTACACATCTGCCCTGCTGGCCGGCACAATCACAAGCGGCTGGACATTTGGAGCAGGCGCCGCTCTGCAGAT |
| | | CCCCTTTGCTATGCAGATGGCCTACCGGTTCAACGGCATCGGAGTGACCCAGAATGTGCTGTACGAGAACCAGAAGC |
| | | TGATCGCCAACCAGTTCAACAGCGCCATCGGCAAGATCCAGGACAGCCTGAGCAGCACAGCAAGCGCCCTGGGAAA |
| | | GCTGCAGGACGTGGTCAACCAGAATGCCCAGGCACTGAACACCCTGGTCAAGCAGCTGTCCTCCAACTTCGGCGCC |
| | | ATCAGCTCTGTGCTGAACGATATCCTGAGCAGACTGGACCCTCCTGAGGCCGAGGTGCAGATCGACAGACTGATCAC |
| | | AGGCAGACTGCAGAGCCTCCAGACATACGTGACCCAGCAGCTGATCAGAGCCGCCGAGATTAGAGCCTCTGCCAAT |
| | | CTGGCCGCCACCAAGATGTCTGAGTGTGTGCTGGGCCAGAGCAAGAGAGTGGACTTTTGCGGCAAGGGCTACCACC |
| | | TGATGAGCTTCCCTCAGTCTGCCCCTCACGGCGTGGTGTTTCTGCACGTGACATATGTGCCCGCTCAAGAGAAGAAT |
| | | TTCACCACCGCTCCAGCCATCTGCCACGACGGCAAAGCCCACTTTCCTAGAGAAGGCGTGTTCGTGTCCAACGGCAC |
| | | CCATTGGTTCGTGACACAGCGGAACTTCTACGAGCCCCAGATCATCACCACCGACAACACCTTCGTGTCTGGCAACT |
| | | GCGACGTCGTGATCGGCATTGTGAACAATACCGTGTACGACCCTCTGCAGCCCGAGCTGGACAGCTTCAAAGAGGAA |
| | | CTGGACAAGTACTTTAAGAACCACACAAGCCCCGACGTGGACCTGGGCGATATCAGCGGAATCAATGCCAGCGTCGT |
| | | GAACATCCAGAAAGAGATCGACCGGCTGAACGAGGTGGCCAAGAATCTGAACGAGAGCCTGATCGACCTGCAAGAA |
| | | CTGGGGAAGTACGAGCAGTACATCAAGTGGCCCTGGTACATCTGGCTGGGCTTTATCGCCGGACTGATTGCCATCGT |
| | | GATGGTCACAATCATGCTGTGTTGCATGACCAGCTGCTGTAGCTGCCTGAAGGGCTGTTGTAGCTGTGGCAGCTGCT |
| | | GCAAGTTCGACGAGGACGATTCTGAGCCCGTGCTGAAGGGCGTGAAACTGCACTACACA |
| 238 | 2A8620 | atgtttgtttttcttgttttattgccactagtctctagtcagtgtgttaatcttacaaccagaactcaattaccccctgcatacactaattctttcacacgtggtgtttattaccctgacaa |
| | | agttttcagatcctcagttttacattcaactcaggatttgttcttacctttcttttccaatgttacttggttccatgctatacatgtctctgggaccaatggtactaagaggtttgataac |
| | | cctgtcctaccatttaatgatggtgtttattttgcttccactgagaagtctaacataataagaggctggatctttggtactactttagattcgaagacccagtccctacttattgttaataa |
| | | cgctactaatgttgttattaaagtctgtgaatttcaattttgtaatgatccatttttgggtgtttattaccacaaaaacaacaaaagttggatggaaagtgagttcagagtttattctagtg |
| | | cgaataattgcacttttgaatatgtctctcagccttttcttatggaccttgaaggaaaacagggtaatttcaaaaatcttagggaatttgtgtttaagaatattgatggttattttaaaata |
| | | tattctaagcacacgcctattaatttagtgcgtgatctccctcagggtttttcggctttagaaccattggtagatttgccaataggtattaacatcactaggtttcaaactttacttgcttt |
| | | acatagaagttatttgactcctggtgattcttcttcaggttggacagctggtgctgcagcttattatgtgggttatcttcaacctaggacttttctattaaaatataatgaaaatggaacca |
| | | ttacagatgctgtagactgtgcacttgaccctctctcagaaacaaagtgtacgttgaaatccttcactgtagaaaaaggaatctatcaaacttctaactttagagtccaaccaacaga |
| | | atctattgttagatttcctaatattacaaacttgtgcccttttggtgaagtttttaacgccaccagatttgcatccgtgtatgcttggaacaggaagagaatcagcaactgtgttgctga |
| | | ttattctgtcctatataattccgcatcattttccacttttaagtgttatggagtgtctcctactaaattaaatgatctctgctttactaatgtctatgcagattcatttgtaattagaggtg |
| | | atgaagtcagacaaatcgctccagggcaaactggaaagattgctgattataattataaattaccagatgattttacaggctgcgttatagcttggaattctaacaatcttgattctaaggtt |
| | | ggtggtaattataattacctgtatagattgtttaggaagtctaatctcaaaccttttgagagagatatttcaactgaaatctatcaggccggtagcacaccttgtaatggtgttgaag |
| | | gttttaattgttactttcctttacaatcatatggtttccaacccactaatggtgttggttaccaaccatacagagtagtagtactttcttttgaacttctacatgcaccagcaactgtttgt |
| | | ggacctaaaaagtctactaatttggttaaaaacaaatgtgtcaatttcaacttcaatggtttaacaggcacaggtgttcttactgagtctaacaaaaagtttctgcctttccaacaattt |
| | | ggcagagacattgctgacactactgatgctgtccgtgatccacagacacttgagattcttgacattacaccatgttcttttggtggtgtcagtgttataacaccaggaacaaatact |
| | | tctaaccaggttgctgttctttatcaggatgttaactgcacagaagtccctgttgctattcatgcagatcaacttactcctacttggcgtgtttattctacaggttctaatgtttttcaaac |
| | | acgtgcaggctgtttaataggggctgaacatgtcaacaactcatatgagtgtgacatacccattggtgcaggtatatgcgctagttatcagactcagactaattctcctcggcggg |
| | | cacgtagtgtagctagtcaatccatcattgcctacactatgtcacttggtgcagaaaattcagttgcttactctaataactctattgccatacccacaaattttactattagtgttacca |
| | | cagaaattctaccagtgtctatgaccaagacatcagtagattgtacaatgtacatttgtggtgattcaactgaatgcagcaatcttttgttgcaatatggcagtttttgtacacaatta |
| | | aaccgtgctttaactgggatagctgttgaacaagacaaaaacacccaagaagtttttgcacaagtcaaacaaatttacaaaacaccaccaattaaagattttggtggttttaatt |
| | | tttcacaaatattaccagatccatcaaaaccaagcaagaggtcatttattgaagatctacttttcaacaaagtgacacttgcagatgctggcttcatcaaacaatatggtgattgc |
| | | cttggtgatattgctgctagggacctcatttgtgcacaaaagtttaacggccttactgttttgccacctttgctcacagatgaaatgattgctcaatacacttctgcactgttagcgggt |
| | | acaatcacttctggttggacctttggtgcaggtgctgcattacaaataccatttgctatgcaaatggcttataggtttaatggtattggagttacacagaatgttctctatgagaacca |
| | | aaaattgattgccaaccaatttaatagtgccattggcaaaattcaagactcactttcttccacagcaagtgcacttggaaaacttcaagatgtggtcaaccaaaatgcacaagc |
| | | tttaaacacgcttgttaaacaacttagctccaattttggtgcaatttcaagtgttttaaatgatatcctttcacgtcttgaccctcccgaggctgaagtgcaaattgataggttgatcac |
| | | aggcagacttcaaagtttgcagacatatgtgactcaacaattaattagagctgcagaaatcagagcttctgctaatcttgctgctactaaaatgtcagagtgtgtacttggacaat |
| | | caaaaagagttgatttttgtggaaagggctatcatcttatgtccttccctcagtcagcacctcatggtgtagtcttcttgcatgtgacttatgtccctgcacaagaaaagaacttcac |
| | | aactgctcctgccatttgtcatgatggaaaagcacactttcctcgtgaaggtgtctttgtttcaaatggcacacactggtttgtaacacaaaggaatttttatgaaccacaaatcatt |
| | | actacagacaacacatttgtgtctggtaactgtgatgttgtaataggaattgtcaacaacacagtttatgatcctttgcaacctgaattagactcattcaaggaggagttagataaa |
| | | tattttaagaatcatacatcaccagatgttgatttaggtgacatctctggcattaatgcttcagttgtaaacattcaaaaagaaattgaccgcctcaatgaggttgccaagaatttaa |
| | | atgaatctctcatcgatctccaagaacttggaaagtatgagcagtatataaaatggccatggtacatttggctaggttttatagctggcttgattgccatagtaatggtgacaattat |
| | | gctttgctgtatgaccagttgctgtagttgtctcaagggctgttgttcttgtggatcctgctgcaaatttgatgaagacgactctgagccagtgctcaaaggagtcaaattacattac |
| | | aca |
| 239 | 2A9420 | ATGTTCGTGTTCCTGGTGCTGCTGCCTCTGGTGTCCAGCCAGTGTGTGAACCTGACCACCAGAACACAGCTGCCTCC |
| | | AGCCTACACCAACAGCTTTACCAGAGGCGTGTACTACCCCGACAAGGTGTTCAGATCCAGCGTGCTGCACTCTACCC |
| | | AGGACCTGTTCCTGCCTTTCTTCAGCAACGTGACCTGGTTCCACGCCATCCACGTGTCCGGCACCAACGGCACCAAG |
| | | AGATTCGACAACCCCGTGCTGCCCTTCAACGACGGGGTGTACTTTGCCAGCACCGAGAAGTCCAACATCATCAGAGG |
| | | CTGGATCTTCGGCACCACACTGGACAGCAAGACCCAGAGCCTGCTGATCGTGAACAACGCCACCAACGTGGTCATCA |
| | | AAGTGTGCGAGTTCCAGTTCTGCAACGACCCCTTCCTGGGCGTCTACTACCACAAGAACAACAAGAGCTGGATGGAA |
| | | AGCGAGTTCCGGGTGTACAGCAGCGCCAACAACTGCACCTTCGAGTACGTGTCCCAGCCTTTCCTGATGGACCTGGA |
| | | AGGCAAGCAGGGCAACTTCAAGAACCTGCGCGAGTTCGTGTTTAAGAACATCGACGGCTACTTCAAGATCTACAGCA |
| | | AGCACACCCCTATCAACCTCGTGCGGGATCTGCCTCAGGGCTTCTCTGCTCTGGAACCCCTGGTGGATCTGCCCATC |
| | | GGCATCAACATCACCCGGTTTCAGACACTGCTGGCCCTGCACAGAAGCTACCTGACACCTGGCGATAGCAGCAGCG |
| | | GCTGGACAGCTGGTGCCGCCGCTTACTACGTGGGCTACCTGCAGCCTAGAACCTTCCTGCTGAAGTACAACGAGAAC |
| | | GGCACCATCACCGACGCCGTGGATTGTGCTCTGGATCCTCTGAGCGAGACAAAGTGCACCCTGAAGTCCTTCACCGT |
| | | GGAAAAGGGCATCTACCAGACCAGCAACTTCCGGGTGCAGCCCACCGAATCCATCGTGCGGTTCCCCAATATCACCA |
| | | ATCTGTGCCCCTTCGGCGAGGTGTTCAACGCCACCAGATTCGCCTCTGTGTACGCCTGGAACCGGAAGCGGATCAG |
| | | CAATTGCGTGGCCGACTACTCCGTGCTGTACAACTCCGCCAGCTTCAGCACCTTCAAGTGCTACGGCGTGTCCCCTA |
| | | CCAAGCTGAACGACCTGTGCTTCACAAACGTGTACGCCGACAGCTTCGTGATCCGGGGAGACGAAGTGCGGCAGAT |
| | | TGCCCCTGGACAGACAGGCAAGATCGCCGACTACAACTACAAGCTGCCCGACGACTTCACCGGCTGTGTGATTGCCT |
| | | GGAACAGCAACAACCTGGACTCCAAAGTCGGCGGCAACTACAATTACCTGTACCGGCTGTTCCGGAAGTCCAATCTG |
| | | AAGCCCTTCGAGCGGGACATCTCCACCGAGATCTATCAGGCCGGCAGCACCCCTTGTAACGGCGTGGAAGGCTTCA |
| | | ACTGCTACTTCCCACTGCAGTCCTACGGCTTTCAGCCCACAAACGGCGTGGGCTATCAGCCCTACAGAGTGGTGGTG |
| | | CTGAGCTTCGAACTGCTGCACGCCCCTGCCACAGTGTGCGGCCCTAAGAAAAGCACCAATCTCGTGAAGAACAAGTG |
| | | CGTGAACTTCAACTTCAACGGCCTGACCGGCACCGGCGTGCTGACAGAGAGCAACAAGAAGTTCCTGCCATTCCAGC |
| | | AGTTTGGCCGGGATATCGCCGATACCACAGACGCCGTTAGAGATCCCCAGACACTGGAAATCCTGGACATCACCCCT |
| | | TGCAGCTTCGGCGGAGTGTCTGTGATCACCCCTGGCACCAACACCAGCAATCAGGTGGCAGTGCTGTACCAGGACG |
| | | TGAACTGTACCGAAGTGCCCGTGGCCATTCACGCCGATCAGCTGACACCTACCTGGCGGGTGTACTCCACCGGCAG |
| | | CAACGTGTTTCAGACCAGAGCCGGCTGTCTGATCGGAGCCGAGCACGTGAACAATAGCTACGAGTGCGACATCCCCA |
| | | TCGGCGCTGGAATCTGCGCCAGCTACCAGACACAGACAAACAGCCCTCGGAGAGCCAGAAGCGTGGCCAGCCAGAG |
| | | CATCATTGCCTACACAATGTCTCTGGGCGCCGAGAACAGCGTGGCCTACTCCAACAACTCTATCGCTATCCCCACCAA |
| | | CTTCACCATCAGCGTGACCACAGAGATCCTGCCTGTGTCCATGACCAAGACCAGCGTGGACTGCACCATGTACATCT |
| | | GCGGCGATTCCACCGAGTGCTCCAACCTGCTGCTGCAGTACGGCAGCTTCTGCACCCAGCTGAATAGAGCCCTGAC |
| | | AGGGATCGCCGTGGAACAGGACAAGAACACCCAAGAGGTGTTCGCCCAAGTGAAGCAGATCTACAAGACCCCTCCTA |
| | | TCAAGGACTTCGGCGGCTTCAATTTCAGCCAGATTCTGCCCGATCCTAGCAAGCCCAGCAAGCGGAGCTTCATCGAG |
| | | GACCTGCTGTTCAACAAAGTGACACTGGCCGACGCCGGCTTCATCAAGCAGTACGGCGATTGTCTGGGCGACATTGC |
| | | CGCCAGGGATCTGATTTGCGCCCAGAAGTTTAACGGACTGACAGTGCTGCCTCCTCTGCTGACCGACGAGATGATCG |
| | | CCCAGTACACATCTGCCCTGCTGGCCGGCACAATCACAAGCGGCTGGACATTTGGAGCAGGCGCCGCTCTGCAGAT |
| | | CCCCTTTGCTATGCAGATGGCCTACCGGTTCAACGGCATCGGAGTGACCCAGAACGTGCTGTACGAGAACCAGAAGC |
| | | TGATCGCCAACCAGTTCAACAGCGCCATCGGCAAGATCCAGGACAGCCTGAGCAGCACAGCAAGCGCCCTGGGAAA |
| | | GCTGCAGGACGTGGTCAACCAGAACGCCCAGGCACTGAACACCCTGGTCAAGCAGCTGTCCTCCAACTTCGGCGCC |
| | | ATCAGCTCTGTGCTGAACGATATCCTGAGCAGACTGGACCCTCCTGAGGCCGAGGTGCAGATCGACAGACTGATCAC |
| | | AGGCAGACTGCAGAGCCTCCAGACATACGTGACCCAGCAGCTGATCAGAGCCGCCGAGATTAGAGCCTCTGCCAAT |
| | | CTGGCCGCCACCAAGATGTCTGAGTGTGTGCTGGGCCAGAGCAAGAGAGTGGACTTTTGCGGCAAGGGCTACCACC |
| | | TGATGAGCTTCCCTCAGTCTGCCCCTCACGGCGTGGTGTTTCTGCACGTGACATACGTGCCCGCTCAAGAGAAGAAT |
| | | TTCACCACCGCTCCAGCCATCTGCCACGACGGCAAAGCCCACTTTCCTAGAGAAGGCGTGTTCGTGTCCAACGGCAC |
| | | CCATTGGTTCGTGACACAGCGGAACTTCTACGAGCCCCAGATCATCACCACCGACAACACCTTCGTGTCTGGCAACT |
| | | GCGACGTCGTGATCGGCATTGTGAACAATACCGTGTACGACCCTCTGCAGCCCGAGCTGGACAGCTTCAAAGAGGAA |
| | | CTGGACAAGTACTTTAAGAACCACACAAGCCCCGACGTGGACCTGGGCGATATCAGCGGAATCAACGCCAGCGTCGT |
| | | GAACATCCAGAAAGAGATCGACCGGCTGAACGAGGTGGCCAAGAATCTGAACGAGAGCCTGATCGACCTGCAAGAA |
| | | CTGGGGAAGTACGAGCAGTACATCAAGTGGCCCTGGTACATCTGGCTGGGCTTTATCGCCGGACTGATTGCCATCGT |
| | | GATGGTCACAATCATGCTGTGTTGCATGACCAGCTGCTGTAGCTGCCTGAAGGGCTGTTGTAGCTGTGGCAGCTGCT |
| | | GCAAGTTCGACGAGGACGATTCTGAGCCCGTGCTGAAGGGCGTGAAACTGCACTACACA |
| 240 | 2A4361 | ATGGGAGTCAAAGTTCTGTTTGCCCTGATCTGCATTGCTGTGGCCGAGGCCAAGAGAGTCCAACCAACAGAATCTATT |
| | | GTTAGATTTCCTAATATTACAAACTTGTGCCCTTTTGGTGAAGTTTTTAACGCCACCAGATTTGCATCCGTGTATGCTTG |
| | | GAACAGGAAGAGAATCAGCAACTGTGTTGCTGATTATTCTGTCCTATATAATTCCGCATCATTTTCCACTTTTAAGTGTT |
| | | ATGGAGTGTCTCCTACTAAATTAAATGATCTCTGCTTTACTAATGTCTATGCAGATTCATTTGTAATTAGAGGTGATGAA |
| | | GTCAGACAAATCGCTCCAGGGCAAACTGGAAAGATTGCTGATTATAATTATAAATTACCAGATGATTTTACAGGCTGCG |
| | | TTATAGCTTGGAATTCTAACAATCTTGATTCTAAGGTTGGTGGTAATTATAATTACCTGTATAGATTGTTTAGGAAGTCT |
| | | AATCTCAAACCTTTTGAGAGAGATATTTCAACTGAAATCTATCAGGCCGGTAGCACACCTTGTAATGGTGTTGAAGGTT |
| | | TTAATTGTTACTTTCCTTTACAATCATATGGTTTCCAACCCACTAATGGTGTTGGTTACCAACCATACAGAGTAGTAGTA |
| | | CTTTCTTTTGAACTTCTACATGCACCAGCAACTGTTTGTGGACCTAAAAAGTCTACTAATTTGGTTAAAAACAAATGTGT |
| | | CAATTTCGGAAGCGGCAGGCTGAGACGAGGCAGCGGCGCCACCAACTTCAGCCTGTTGAAGCAGGCCGGCGACGT |
| | | GGAGGAAAACCCCGGCCCCATGGGAGTCAAAGTTCTGTTTGCCCTGATCTGCATTGCTGTGGCCGAGGCCAAGAGA |
| | | GTCCAACCAACAGAATCTATTGTTAGATTTCCTAATATTACAAACTTGTGCCCTTTTGGTGAAGTTTTTAACGCCACCAG |
| | | ATTTGCATCCGTGTATGCTTGGAACAGGAAGAGAATCAGCAACTGTGTTGCTGATTATTCTGTCCTATATAATTCCGCA |
| | | TCATTTTCCACTTTTAAGTGTTATGGAGTGTCTCCTACTAAATTAAATGATCTCTGCTTTACTAATGTCTATGCAGATTCA |
| | | TTTGTAATTAGAGGTGATGAAGTCAGACAAATCGCTCCAGGGCAAACTGGAAAGATTGCTGATTATAATTATAAATTAC |
| | | CAGATGATTTTACAGGCTGCGTTATAGCTTGGAATTCTAACAATCTTGATTCTAAGGTTGGTGGTAATTATAATTACCTG |
| | | TATAGATTGTTTAGGAAGTCTAATCTCAAACCTTTTGAGAGAGATATTTCAACTGAAATCTATCAGGCCGGTAGCACAC |
| | | CTTGTAATGGTGTTGAAGGTTTTAATTGTTACTTTCCTTTACAATCATATGGTTTCCAACCCACTAATGGTGTTGGTTAC |
| | | CAACCATACAGAGTAGTAGTACTTTCTTTTGAACTTCTACATGCACCAGCAACTGTTTGTGGACCTAAAAAGTCTACTA |
| | | ATTTGGTTAAAAACAAATGTGTCAATTTC |
| 241 | 2A5361 | ATGGGAGTCAAAGTTCTGTTTGCCCTGATCTGCATTGCTGTGGCCGAGGCCAAGAGAGTCCAACCAACAGAATCTATT |
| | | GTTAGATTTCCTAATATTACAAACTTGTGCCCTTTTGGTGAAGTTTTTAACGCCACCAGATTTGCATCCGTGTATGCTTG |
| | | GAACAGGAAGAGAATCAGCAACTGTGTTGCTGATTATTCTGTCCTATATAATTCCGCATCATTTTCCACTTTTAAGTGTT |
| | | ATGGAGTGTCTCCTACTAAATTAAATGATCTCTGCTTTACTAATGTCTATGCAGATTCATTTGTAATTAGAGGTGATGAA |
| | | GTCAGACAAATCGCTCCAGGGCAAACTGGAAAGATTGCTGATTATAATTATAAATTACCAGATGATTTTACAGGCTGCG |
| | | TTATAGCTTGGAATTCTAACAATCTTGATTCTAAGGTTGGTGGTAATTATAATTACCTGTATAGATTGTTTAGGAAGTCT |
| | | AATCTCAAACCTTTTGAGAGAGATATTTCAACTGAAATCTATCAGGCCGGTAGCACACCTTGTAATGGTGTTGAAGGTT |
| | | TTAATTGTTACTTTCCTTTACAATCATATGGTTTCCAACCCACTAATGGTGTTGGTTACCAACCATACAGAGTAGTAGTA |
| | | CTTTCTTTTGAACTTCTACATGCACCAGCAACTGTTTGTGGACCTAAAAAGTCTACTAATTTGGTTAAAAACAAATGTGT |
| | | CAATTTCGGAAGCGGCAGGCTGAGACGAGGCAGCGGCGCCACCAACTTCAGCCTGTTGAAGCAGGCCGGCGACGT |
| | | GGAGGAAAACCCCGGCCCCATGGGAGTCAAAGTTCTGTTTGCCCTGATCTGCATTGCTGTGGCCGAGGCCAAGAGA |
| | | GTCCAACCAACAGAATCTATTGTTAGATTTCCTAATATTACAAACTTGTGCCCTTTTGGTGAAGTTTTTAACGCCACCAG |
| | | ATTTGCATCCGTGTATGCTTGGAACAGGAAGAGAATCAGCAACTGTGTTGCTGATTATTCTGTCCTATATAATTCCGCA |
| | | TCATTTTCCACTTTTAAGTGTTATGGAGTGTCTCCTACTAAATTAAATGATCTCTGCTTTACTAATGTCTATGCAGATTCA |
| | | TTTGTAATTAGAGGTGATGAAGTCAGACAAATCGCTCCAGGGCAAACTGGAAAGATTGCTGATTATAATTATAAATTAC |
| | | CAGATGATTTTACAGGCTGCGTTATAGCTTGGAATTCTAACAATCTTGATTCTAAGGTTGGTGGTAATTATAATTACCTG |
| | | TATAGATTGTTTAGGAAGTCTAATCTCAAACCTTTTGAGAGAGATATTTCAACTGAAATCTATCAGGCCGGTAGCACAC |
| | | CTTGTAATGGTGTTGAAGGTTTTAATTGTTACTTTCCTTTACAATCATATGGTTTCCAACCCACTAATGGTGTTGGTTAC |
| | | CAACCATACAGAGTAGTAGTACTTTCTTTTGAACTTCTACATGCACCAGCAACTGTTTGTGGACCTAAAAAGTCTACTA |
| | | ATTTGGTTAAAAACAAATGTGTCAATTTCGGAAGCGGCAGGCTGAGACGAGGCAGCGGCGCCACCAACTTCAGCCTG |
| | | TTGAAGCAGGCCGGCGACGTGGAGGAAAACCCCGGCCCCATGGGAGTCAAAGTTCTGTTTGCCCTGATCTGCATTG |
| | | CTGTGGCCGAGGCCAAGAGAGTCCAACCAACAGAATCTATTGTTAGATTTCCTAATATTACAAACTTGTGCCCTTTTGG |
| | | TGAAGTTTTTAACGCCACCAGATTTGCATCCGTGTATGCTTGGAACAGGAAGAGAATCAGCAACTGTGTTGCTGATTAT |
| | | TCTGTCCTATATAATTCCGCATCATTTTCCACTTTTAAGTGTTATGGAGTGTCTCCTACTAAATTAAATGATCTCTGCTTT |
| | | ACTAATGTCTATGCAGATTCATTTGTAATTAGAGGTGATGAAGTCAGACAAATCGCTCCAGGGCAAACTGGAAAGATT |
| | | GCTGATTATAATTATAAATTACCAGATGATTTTACAGGCTGCGTTATAGCTTGGAATTCTAACAATCTTGATTCTAAGGT |
| | | TGGTGGTAATTATAATTACCTGTATAGATTGTTTAGGAAGTCTAATCTCAAACCTTTTGAGAGAGATATTTCAACTGAAA |
| | | TCTATCAGGCCGGTAGCACACCTTGTAATGGTGTTGAAGGTTTTAATTGTTACTTTCCTTTACAATCATATGGTTTCCAA |
| | | CCCACTAATGGTGTTGGTTACCAACCATACAGAGTAGTAGTACTTTCTTTTGAACTTCTACATGCACCAGCAACTGTTT |
| | | GTGGACCTAAAAAGTCTACTAATTTGGTTAAAAACAAATGTGTCAATTTC |
| 242 | 2A3461 | ATGGGAGTCAAAGTTCTGTTTGCCCTGATCTGCATTGCTGTGGCCGAGGCCAAGAGAGTCCAACCAACAGAATCTATT |
| | | GTTAGATTTCCTAATATTACAAACTTGTGCCCTTTTGGTGAAGTTTTTAACGCCACCAGATTTGCATCCGTGTATGCTTG |
| | | GAACAGGAAGAGAATCAGCAACTGTGTTGCTGATTATTCTGTCCTATATAATTCCGCATCATTTTCCACTTTTAAGTGTT |
| | | ATGGAGTGTCTCCTACTAAATTAAATGATCTCTGCTTTACTAATGTCTATGCAGATTCATTTGTAATTAGAGGTGATGAA |
| | | GTCAGACAAATCGCTCCAGGGCAAACTGGAAAGATTGCTGATTATAATTATAAATTACCAGATGATTTTACAGGCTGCG |
| | | TTATAGCTTGGAATTCTAACAATCTTGATTCTAAGGTTGGTGGTAATTATAATTACCTGTATAGATTGTTTAGGAAGTCT |
| | | AATCTCAAACCTTTTGAGAGAGATATTTCAACTGAAATCTATCAGGCCGGTAGCACACCTTGTAATGGTGTTGAAGGTT |
| | | TTAATTGTTACTTTCCTTTACAATCATATGGTTTCCAACCCACTAATGGTGTTGGTTACCAACCATACAGAGTAGTAGTA |
| | | CTTTCTTTTGAACTTCTACATGCACCAGCAACTGTTTGTGGACCTAAAAAGTCTACTAATTTGGTTAAAAACAAATGTGT |
| | | CAATTTCGGAAGCGGCAGGCTGAGACGAGGCAGCGGCGCCACCAACTTCAGCCTGTTGAAGCAGGCCGGCGACGT |
| | | GGAGGAAAACCCCGGCCCCAGAGTCCAACCAACAGAATCTATTGTTAGATTTCCTAATATTACAAACTTGTGCCCTTTT |
| | | GGTGAAGTTTTTAACGCCACCAGATTTGCATCCGTGTATGCTTGGAACAGGAAGAGAATCAGCAACTGTGTTGCTGAT |
| | | TATTCTGTCCTATATAATTCCGCATCATTTTCCACTTTTAAGTGTTATGGAGTGTCTCCTACTAAATTAAATGATCTCTGC |
| | | TTTACTAATGTCTATGCAGATTCATTTGTAATTAGAGGTGATGAAGTCAGACAAATCGCTCCAGGGCAAACTGGAAAGA |
| | | TTGCTGATTATAATTATAAATTACCAGATGATTTTACAGGCTGCGTTATAGCTTGGAATTCTAACAATCTTGATTCTAAG |
| | | GTTGGTGGTAATTATAATTACCTGTATAGATTGTTTAGGAAGTCTAATCTCAAACCTTTTGAGAGAGATATTTCAACTGA |
| | | AATCTATCAGGCCGGTAGCACACCTTGTAATGGTGTTGAAGGTTTTAATTGTTACTTTCCTTTACAATCATATGGTTTCC |
| | | AACCCACTAATGGTGTTGGTTACCAACCATACAGAGTAGTAGTACTTTCTTTTGAACTTCTACATGCACCAGCAACTGT |
| | | TTGTGGACCTAAAAAGTCTACTAATTTGGTTAAAAACAAATGTGTCAATTTC |
| 243 | 2A4461 | ATGGGAGTCAAAGTTCTGTTTGCCCTGATCTGCATTGCTGTGGCCGAGGCCAAGAGAGTCCAACCAACAGAATCTATT |
| | | GTTAGATTTCCTAATATTACAAACTTGTGCCCTTTTGGTGAAGTTTTTAACGCCACCAGATTTGCATCCGTGTATGCTTG |
| | | GAACAGGAAGAGAATCAGCAACTGTGTTGCTGATTATTCTGTCCTATATAATTCCGCATCATTTTCCACTTTTAAGTGTT |
| | | ATGGAGTGTCTCCTACTAAATTAAATGATCTCTGCTTTACTAATGTCTATGCAGATTCATTTGTAATTAGAGGTGATGAA |
| | | GTCAGACAAATCGCTCCAGGGCAAACTGGAAAGATTGCTGATTATAATTATAAATTACCAGATGATTTTACAGGCTGCG |
| | | TTATAGCTTGGAATTCTAACAATCTTGATTCTAAGGTTGGTGGTAATTATAATTACCTGTATAGATTGTTTAGGAAGTCT |
| | | AATCTCAAACCTTTTGAGAGAGATATTTCAACTGAAATCTATCAGGCCGGTAGCACACCTTGTAATGGTGTTGAAGGTT |
| | | TTAATTGTTACTTTCCTTTACAATCATATGGTTTCCAACCCACTAATGGTGTTGGTTACCAACCATACAGAGTAGTAGTA |
| | | CTTTCTTTTGAACTTCTACATGCACCAGCAACTGTTTGTGGACCTAAAAAGTCTACTAATTTGGTTAAAAACAAATGTGT |
| | | CAATTTCGGAAGCGGCAGGCTGAGACGAGGCAGCGGCGCCACCAACTTCAGCCTGTTGAAGCAGGCCGGCGACGT |
| | | GGAGGAAAACCCCGGCCCCAGAGTCCAACCAACAGAATCTATTGTTAGATTTCCTAATATTACAAACTTGTGCCCTTTT |
| | | GGTGAAGTTTTTAACGCCACCAGATTTGCATCCGTGTATGCTTGGAACAGGAAGAGAATCAGCAACTGTGTTGCTGAT |
| | | TATTCTGTCCTATATAATTCCGCATCATTTTCCACTTTTAAGTGTTATGGAGTGTCTCCTACTAAATTAAATGATCTCTGC |
| | | TTTACTAATGTCTATGCAGATTCATTTGTAATTAGAGGTGATGAAGTCAGACAAATCGCTCCAGGGCAAACTGGAAAGA |
| | | TTGCTGATTATAATTATAAATTACCAGATGATTTTACAGGCTGCGTTATAGCTTGGAATTCTAACAATCTTGATTCTAAG |
| | | GTTGGTGGTAATTATAATTACCTGTATAGATTGTTTAGGAAGTCTAATCTCAAACCTTTTGAGAGAGATATTTCAACTGA |
| | | AATCTATCAGGCCGGTAGCACACCTTGTAATGGTGTTGAAGGTTTTAATTGTTACTTTCCTTTACAATCATATGGTTTCC |
| | | AACCCACTAATGGTGTTGGTTACCAACCATACAGAGTAGTAGTACTTTCTTTTGAACTTCTACATGCACCAGCAACTGT |
| | | TTGTGGACCTAAAAAGTCTACTAATTTGGTTAAAAACAAATGTGTCAATTTCGGAAGCGGCAGGCTGAGACGAGGCAG |
| | | CGGCGCCACCAACTTCAGCCTGTTGAAGCAGGCCGGCGACGTGGAGGAAAACCCCGGCCCCAGAGTCCAACCAACA |
| | | GAATCTATTGTTAGATTTCCTAATATTACAAACTTGTGCCCTTTTGGTGAAGTTTTTAACGCCACCAGATTTGCATCCGT |
| | | GTATGCTTGGAACAGGAAGAGAATCAGCAACTGTGTTGCTGATTATTCTGTCCTATATAATTCCGCATCATTTTCCACT |
| | | TTTAAGTGTTATGGAGTGTCTCCTACTAAATTAAATGATCTCTGCTTTACTAATGTCTATGCAGATTCATTTGTAATTAGA |
| | | GGTGATGAAGTCAGACAAATCGCTCCAGGGCAAACTGGAAAGATTGCTGATTATAATTATAAATTACCAGATGATTTTA |
| | | CAGGCTGCGTTATAGCTTGGAATTCTAACAATCTTGATTCTAAGGTTGGTGGTAATTATAATTACCTGTATAGATTGTTT |
| | | AGGAAGTCTAATCTCAAACCTTTTGAGAGAGATATTTCAACTGAAATCTATCAGGCCGGTAGCACACCTTGTAATGGTG |
| | | TTGAAGGTTTTAATTGTTACTTTCCTTTACAATCATATGGTTTCCAACCCACTAATGGTGTTGGTTACCAACCATACAGA |
| | | GTAGTAGTACTTTCTTTTGAACTTCTACATGCACCAGCAACTGTTTGTGGACCTAAAAAGTCTACTAATTTGGTTAAAAA |
| | | CAAATGTGTCAATTTC |
| 244 | 2A5461 | ATGGGAGTCAAAGTTCTGTTTGCCCTGATCTGCATTGCTGTGGCCGAGGCCAAGAGAGTCCAACCAACAGAATCTATT |
| | | GTTAGATTTCCTAATATTACAAACTTGTGCCCTTTTGGTGAAGTTTTTAACGCCACCAGATTTGCATCCGTGTATGCTTG |
| | | GAACAGGAAGAGAATCAGCAACTGTGTTGCTGATTATTCTGTCCTATATAATTCCGCATCATTTTCCACTTTTAAGTGTT |
| | | ATGGAGTGTCTCCTACTAAATTAAATGATCTCTGCTTTACTAATGTCTATGCAGATTCATTTGTAATTAGAGGTGATGAA |
| | | GTCAGACAAATCGCTCCAGGGCAAACTGGAAAGATTGCTGATTATAATTATAAATTACCAGATGATTTTACAGGCTGCG |
| | | TTATAGCTTGGAATTCTAACAATCTTGATTCTAAGGTTGGTGGTAATTATAATTACCTGTATAGATTGTTTAGGAAGTCT |
| | | AATCTCAAACCTTTTGAGAGAGATATTTCAACTGAAATCTATCAGGCCGGTAGCACACCTTGTAATGGTGTTGAAGGTT |
| | | TTAATTGTTACTTTCCTTTACAATCATATGGTTTCCAACCCACTAATGGTGTTGGTTACCAACCATACAGAGTAGTAGTA |
| | | CTTTCTTTTGAACTTCTACATGCACCAGCAACTGTTTGTGGACCTAAAAAGTCTACTAATTTGGTTAAAAACAAATGTGT |
| | | CAATTTCGGAAGCGGCAGGCTGAGACGAGGCAGCGGCGCCACCAACTTCAGCCTGTTGAAGCAGGCCGGCGACGT |
| | | GGAGGAAAACCCCGGCCCCGGCCTCGAACAACTCGAAAGCATTATCAATTTTGAAAAACTCACAGAATGGACCAGTG |
| | | GACTCGAGCAACTCGAGAGTATCATTAATTTCGAAAAGCTCACAGAGTGGACAAGCGGCCTGGAGCAGCTGGAGAGC |
| | | ATCATAAACTTCGAGAAGCTGACCGAGTGGACCAGC |
| 245 | 2A6461 | ATGGGAGTCAAAGTTCTGTTTGCCCTGATCTGCATTGCTGTGGCCGAGGCCAAGAGAGTCCAACCAACAGAATCTATT |
| | | GTTAGATTTCCTAATATTACAAACTTGTGCCCTTTTGGTGAAGTTTTTAACGCCACCAGATTTGCATCCGTGTATGCTTG |
| | | GAACAGGAAGAGAATCAGCAACTGTGTTGCTGATTATTCTGTCCTATATAATTCCGCATCATTTTCCACTTTTAAGTGTT |
| | | ATGGAGTGTCTCCTACTAAATTAAATGATCTCTGCTTTACTAATGTCTATGCAGATTCATTTGTAATTAGAGGTGATGAA |
| | | GTCAGACAAATCGCTCCAGGGCAAACTGGAAAGATTGCTGATTATAATTATAAATTACCAGATGATTTTACAGGCTGCG |
| | | TTATAGCTTGGAATTCTAACAATCTTGATTCTAAGGTTGGTGGTAATTATAATTACCTGTATAGATTGTTTAGGAAGTCT |
| | | AATCTCAAACCTTTTGAGAGAGATATTTCAACTGAAATCTATCAGGCCGGTAGCACACCTTGTAATGGTGTTGAAGGTT |
| | | TTAATTGTTACTTTCCTTTACAATCATATGGTTTCCAACCCACTAATGGTGTTGGTTACCAACCATACAGAGTAGTAGTA |
| | | CTTTCTTTTGAACTTCTACATGCACCAGCAACTGTTTGTGGACCTAAAAAGTCTACTAATTTGGTTAAAAACAAATGTGT |
| | | CAATTTCCGGCGGAAGCGGAGAAGCGGCAGCGGCGCCACCAACTTCAGCCTGTTGAAGCAGGCCGGCGACGTGGA |
| | | GGAAAACCCCGGCCCCATGGGAGTCAAAGTTCTGTTTGCCCTGATCTGCATTGCTGTGGCCGAGGCCAAGAGAGTCC |
| | | AACCAACAGAATCTATTGTTAGATTTCCTAATATTACAAACTTGTGCCCTTTTGGTGAAGTTTTTAACGCCACCAGATTT |
| | | GCATCCGTGTATGCTTGGAACAGGAAGAGAATCAGCAACTGTGTTGCTGATTATTCTGTCCTATATAATTCCGCATCAT |
| | | TTTCCACTTTTAAGTGTTATGGAGTGTCTCCTACTAAATTAAATGATCTCTGCTTTACTAATGTCTATGCAGATTCATTTG |
| | | TAATTAGAGGTGATGAAGTCAGACAAATCGCTCCAGGGCAAACTGGAAAGATTGCTGATTATAATTATAAATTACCAGA |
| | | TGATTTTACAGGCTGCGTTATAGCTTGGAATTCTAACAATCTTGATTCTAAGGTTGGTGGTAATTATAATTACCTGTATA |
| | | GATTGTTTAGGAAGTCTAATCTCAAACCTTTTGAGAGAGATATTTCAACTGAAATCTATCAGGCCGGTAGCACACCTTG |
| | | TAATGGTGTTGAAGGTTTTAATTGTTACTTTCCTTTACAATCATATGGTTTCCAACCCACTAATGGTGTTGGTTACCAAC |
| | | CATACAGAGTAGTAGTACTTTCTTTTGAACTTCTACATGCACCAGCAACTGTTTGTGGACCTAAAAAGTCTACTAATTTG |
| | | GTTAAAAACAAATGTGTCAATTTC |
| 246 | 2A6481 | ATAGGTCTCACATGTTTGTTTTCTTAGTTTTATTACCTTTAGTTTCTTCCCAGTGTGTTAATTTAACTACTAGAACTCAAT |
| | | TACCTCCTGCTTATACTAATTCTTTTACTAGAGGAGTGTATTATCCTGATAAGGTTTTTAGATCTTCTGTTCTGCACTCT |
| | | ACTCAGGATTTGTTCTTGCCCTTCTTCTCTAACGTGACCTGGTTCCACGCTATTCACGTTTCTGGAACCAACGGCACTA |
| | | AGAGATTTGATAATCCCGTTTTGCCTTTTAACGACGGAGTTTATTTCGCCTCTACCGAAAAATCTAACATCATTAGGGG |
| | | CTGGATCTTTGGAACTACCCTGGATTCTAAAACTCAATCTTTATTGATTGTGAATAACGCTACCAACGTTGTGATTAAAG |
| | | TTTGTGAATTTCAATTTTGTAACGATCCTTTTCTGGGAGTTTATTATCATAAAAACAATAAATCTTGGATGGAATCTGAAT |
| | | TCCGCGTTTATTCTTCTGCCAATAACTGTACCTTTGAGTACGTTTCTCAACCTTTCTTAATGGATTTAGAAGGAAAACAA |
| | | GGCAATTTCAAAAACTTAAGAGAATTTGTTTTTAAAAATATTGACGGATATTTTAAAATTTATTCTAAGCATACTCCTATT |
| | | AATTTAGTGAGAGACTTACCCCAAGGATTTTCTGCTTTAGAACCTTTAGTTGACTTACCTATTGGAATCAATATCACTAG |
| | | ATTTCAAACTTTATTAGCTTTACATAGATCTTATTTAACTCCTGGAGATTCTTCCTCCGGCTGGACAGCCGGAGCCGCT |
| | | GCTTATTACGTTGGATATTTACAGCCTAGAACTTTTCTGCTGAAATATAACGAAAACGGAACCATTACCGACGCCGTTG |
| | | ACTGTGCTCTGGATCCTTTATCTGAAACCAAGTGTACTTTAAAATCTTTTACCGTTGAAAAAGGAATTTATCAAACTTCT |
| | | AATTTTAGAGTTCAACCTACTGAATCTATCGTTAGATTCCCTAATATTACTAATTTGTGTCCCTTTGGAGAGGTTTTCAA |
| | | CGCTACTAGATTTGCCTCTGTTTACGCTTGGAATCGCAAGAGAATTTCTAATTGTGTGGCCGATTACTCCGTTTTGTAT |
| | | AACTCTGCTTCTTTCTCTACTTTTAAGTGTTACGGAGTTTCCCCTACTAAACTGAACGATTTGTGTTTTACTAACGTTTA |
| | | CGCTGATTCTTTTGTTATTAGAGGCGACGAAGTTAGACAAATTGCTCCTGGACAAACTGGAAAGATTGCTGACTATAAC |
| | | TATAAACTGCCTGACGATTTTACCGGCTGTGTTATTGCTTGGAATTCTAATAATTTAGATTCTAAAGTTGGAGGAAATTA |
| | | TAATTATTTATATAGATTATTTAGAAAATCTAATTTAAAACCTTTTGAAAGAGATATTTCCACTGAGATTTACCAGGCTGG |
| | | ATCTACTCCTTGTAACGGCGTTGAGGGATTTAATTGTTATTTTCCTTTACAATCTTACGGATTTCAACCTACCAACGGAG |
| | | TTGGATATCAACCTTACAGAGTTGTGGTGCTGTCTTTTGAATTATTACACGCTCCCGCTACTGTTTGCGGCCCTAAAAA |
| | | GTCTACTAATCTGGTTAAGAATAAGTGTGTGAATTTTAATTTCAACGGATTGACCGGAACTGGAGTTCTGACTGAATCT |
| | | AATAAAAAATTTTTACCCTTTCAACAATTTGGAAGGGATATCGCTGATACTACTGACGCTGTTAGAGATCCTCAAACTTT |
| | | AGAAATCCTGGATATTACTCCTTGTTCTTTTGGAGGAGTTTCCGTTATTACTCCTGGCACTAATACTTCTAATCAAGTTG |
| | | CTGTTTTATATCAAGACGTTAATTGCACCGAAGTTCCTGTTGCTATTCACGCTGATCAGTTAACTCCTACTTGGAGAGT |
| | | TTATTCTACTGGATCTAACGTTTTTCAAACCAGAGCTGGCTGTTTAATCGGAGCTGAACACGTGAATAATTCTTACGAG |
| | | TGCGATATTCCCATCGGAGCTGGAATTTGTGCCTCTTATCAAACTCAAACCAATTCTCCTAGGAGGGCTAGATCCGTT |
| | | GCCTCTCAATCTATTATTGCTTATACCATGTCTTTAGGAGCTGAAAATTCCGTTGCTTACTCTAACAATTCTATTGCTAT |
| | | TCCTACTAACTTTACTATTTCTGTTACTACTGAAATTCTGCCCGTGTCCATGACTAAAACTTCTGTTGATTGTACTATGT |
| | | ATATTTGTGGAGATTCCACTGAGTGTTCTAACCTGTTATTACAATACGGATCTTTTTGTACTCAATTAAATAGAGCTCTG |
| | | ACTGGAATTGCTGTTGAACAGGACAAAAATACTCAAGAAGTTTTTGCCCAAGTTAAACAGATTTATAAAACTCCTCCTAT |
| | | TAAAGATTTTGGAGGATTTAATTTCTCTCAAATTTTACCTGACCCTTCTAAGCCTTCCAAAAGGTCTTTCATCGAAGATT |
| | | TATTATTTAATAAAGTTACCTTAGCCGACGCTGGATTTATTAAGCAATACGGAGATTGCTTAGGCGATATCGCTGCTAG |
| | | AGACTTAATTTGTGCTCAAAAATTTAACGGATTAACTGTTTTACCTCCCCTGTTAACTGACGAAATGATCGCTCAATATA |
| | | CTTCTGCTCTGTTAGCTGGAACTATTACTTCTGGCTGGACTTTTGGAGCTGGAGCTGCTTTACAAATTCCCTTTGCTAT |
| | | GCAGATGGCTTATAGATTTAACGGAATTGGAGTTACTCAAAACGTTTTATACGAAAATCAAAAATTAATTGCTAATCAAT |
| | | TTAATTCTGCTATTGGAAAAATTCAAGATTCCCTGTCTTCTACTGCTTCCGCTCTGGGAAAATTACAAGACGTTGTTAAT |
| | | CAAAACGCTCAAGCTTTGAATACCTTAGTTAAACAATTATCTTCCAATTTTGGAGCTATTTCTTCTGTTTTAAACGACATT |
| | | TTATCTAGATTAGATCCCCCTGAAGCTGAAGTTCAGATTGACAGATTGATTACCGGAAGATTACAATCTTTACAAACTTA |
| | | CGTTACTCAACAACTGATTAGAGCTGCTGAAATTAGAGCCTCTGCTAATTTAGCTGCTACTAAGATGTCTGAGTGTGTT |
| | | TTAGGACAGTCCAAAAGAGTTGATTTTTGTGGAAAAGGCTATCATCTGATGTCTTTTCCTCAATCTGCTCCTCACGGAG |
| | | TTGTGTTTTTACACGTTACTTACGTTCCTGCTCAAGAGAAGAATTTTACTACTGCTCCTGCTATTTGTCACGACGGAAAA |
| | | GCTCATTTTCCTAGAGAAGGAGTTTTTGTTTCTAACGGAACTCATTGGTTTGTTACTCAAAGAAACTTTTACGAACCTCA |
| | | GATTATTACCACTGATAATACCTTTGTTTCTGGCAATTGTGACGTGGTTATTGGAATTGTTAATAATACTGTTTACGATC |
| | | CTTTACAACCCGAACTGGATTCTTTTAAAGAAGAATTAGATAAATACTTTAAAAATCATACTTCTCCCGACGTTGATTTA |
| | | GGAGATATCTCTGGAATTAACGCTTCTGTTGTGAATATTCAAAAGGAAATTGATAGATTAAACGAAGTGGCTAAAAATT |
| | | TAAACGAGTCTTTAATTGATTTACAGGAACTGGGAAAATACGAACAATATATTAAGTGGCCTTGGTACATTTGGTTAGG |
| | | CTTTATTGCTGGACTGATTGCTATTGTGATGGTTACTATCATGTTGTGTTGCATGACTTCTTGTTGTTCCTGTTTAAAAG |
| | | GCTGTTGCTCTTGTGGATCTTGTTGCAAATTTGACGAAGACGATTCTGAACCTGTTCTGAAAGGAGTTAAACTGCATTA |
| | | TACTTAAATGAGACCATA |
| 247 | 2A7481 | ATAGGTCTCACATGTTCGTGTTCTTAGTGCTGTTACCCCTGGTTTCTTCTCAGTGTGTCAATTTAACCACCAGAACCCA |
| | | ACTGCCTCCTGCCTATACTAATTCCTTTACCCGCGGAGTTTACTATCCCGATAAGGTTTTTCGCTCCTCTGTTTTACAC |
| | | TCTACTCAGGACTTATTCTTACCCTTCTTCTCCAACGTTACTTGGTTTCACGCCATTCACGTTTCTGGAACCAACGGAA |
| | | CTAAGAGATTCGATAATCCTGTGTTACCTTTCAACGACGGAGTTTATTTTGCTTCCACTGAAAAATCCAATATCATTAGG |
| | | GGCTGGATCTTTGGAACCACTTTAGATTCCAAGACTCAATCTTTATTAATTGTGAATAACGCCACCAACGTGGTTATCA |
| | | AAGTGTGTGAATTCCAATTTTGTAACGACCCTTTCTTAGGCGTGTATTATCACAAAAATAATAAATCTTGGATGGAATCT |
| | | GAATTTCGCGTTTACTCTTCCGCTAACAACTGCACTTTTGAGTACGTTTCCCAGCCTTTTCTGATGGATCTGGAAGGCA |
| | | AACAGGGAAATTTTAAAAATCTGAGAGAATTTGTTTTTAAGAACATTGACGGATATTTCAAGATCTATTCTAAACACACT |
| | | CCCATCAATTTGGTTAGAGATTTACCTCAGGGCTTTTCTGCTTTAGAACCTTTAGTTGACCTGCCTATTGGAATCAACA |
| | | TTACTAGATTTCAAACTCTGTTAGCTCTGCATAGATCTTATTTAACTCCTGGAGATTCCTCCTCCGGCTGGACCGCCGG |
| | | AGCTGCTGCTTATTACGTTGGCTATTTACAACCCAGAACTTTTCTGTTAAAATATAACGAAAACGGCACCATCACTGAC |
| | | GCCGTGGATTGTGCTTTAGATCCCTTATCCGAAACTAAGTGTACTCTGAAATCCTTCACTGTTGAAAAAGGAATTTATC |
| | | AGACCTCTAATTTTAGAGTGCAACCTACCGAATCTATTGTTAGATTTCCTAATATTACCAACTTGTGCCCTTTTGGCGAA |
| | | GTTTTCAACGCTACTAGATTTGCTTCTGTTTACGCTTGGAATAGAAAAAGAATTTCTAACTGTGTGGCTGACTATTCCGT |
| | | GTTATACAATTCTGCCTCTTTTTCTACTTTTAAGTGTTACGGAGTGTCTCCCACTAAACTGAACGATTTGTGCTTTACCA |
| | | ACGTTTACGCTGATTCTTTTGTTATTAGAGGCGACGAGGTTCGCCAGATTGCCCCTGGCCAGACTGGCAAAATTGCTG |
| | | ACTATAACTATAAATTACCTGACGATTTTACCGGCTGCGTTATCGCTTGGAACTCTAATAATCTGGATTCTAAAGTTGGA |
| | | GGAAACTACAATTATTTATATAGATTATTTAGAAAATCTAATTTAAAACCCTTCGAGAGGGATATCTCTACTGAAATCTA |
| | | CCAAGCTGGATCTACTCCTTGCAACGGCGTTGAAGGATTTAATTGTTATTTTCCTTTACAATCTTACGGATTCCAACCT |
| | | ACTAACGGAGTTGGATATCAGCCTTATAGAGTGGTGGTTTTATCTTTCGAATTATTACACGCTCCCGCTACTGTGTGTG |
| | | GACCCAAAAAGTCTACTAATTTAGTTAAAAATAAGTGTGTTAATTTCAATTTTAACGGACTCACTGGCACTGGAGTGTTA |
| | | ACTGAATCTAATAAAAAGTTCTTACCTTTCCAACAGTTCGGACGCGACATTGCCGACACCACTGACGCTGTGAGAGAT |
| | | CCTCAAACCTTAGAGATTCTGGATATTACCCCTTGTTCCTTTGGAGGCGTTTCCGTTATCACTCCCGGAACTAACACTT |
| | | CTAATCAGGTTGCCGTTTTATATCAGGACGTTAATTGCACTGAAGTTCCTGTTGCTATTCACGCTGATCAACTGACTCC |
| | | TACTTGGAGAGTTTATTCCACTGGATCTAACGTTTTTCAGACTCGCGCCGGCTGTTTAATTGGAGCTGAACACGTGAA |
| | | CAACTCCTACGAGTGCGATATTCCTATTGGCGCTGGCATCTGTGCTTCTTATCAAACTCAAACTAATTCTCCCAGGAG |
| | | GGCTCGCTCTGTTGCTTCCCAATCTATCATTGCTTATACTATGTCCTTAGGCGCTGAAAATTCTGTTGCTTATTCCAAC |
| | | AATTCTATTGCTATTCCCACCAATTTTACTATTTCTGTGACTACTGAAATTTTACCTGTTTCCATGACTAAGACTTCTGTT |
| | | GATTGTACTATGTATATCTGCGGAGATTCTACTGAGTGTTCCAACCTCTTATTACAGTACGGATCTTTTTGCACTCAGTT |
| | | AAATAGAGCTCTGACTGGAATCGCTGTTGAACAAGACAAAAACACCCAAGAAGTGTTTGCTCAGGTGAAGCAAATTTA |
| | | CAAGACACCTCCTATTAAGGATTTTGGAGGATTCAATTTTTCCCAGATTTTACCTGATCCTTCTAAACCTTCTAAACGCT |
| | | CCTTTATTGAAGATTTACTGTTCAATAAAGTGACTCTGGCTGACGCTGGCTTCATCAAGCAATACGGCGATTGTTTAGG |
| | | AGACATCGCTGCTAGAGACTTAATTTGTGCTCAAAAATTCAACGGACTGACTGTTCTGCCCCCTTTACTGACCGACGA |
| | | GATGATCGCCCAATATACTTCTGCCCTGTTAGCTGGCACCATTACTTCTGGCTGGACCTTTGGAGCTGGCGCTGCTTT |
| | | ACAAATTCCTTTCGCCATGCAGATGGCTTATCGCTTTAACGGAATTGGCGTTACCCAAAACGTGTTATACGAGAATCAA |
| | | AAATTAATCGCCAATCAATTTAATTCTGCTATTGGCAAGATTCAAGATTCTTTATCTTCTACTGCTTCCGCCCTGGGCAA |
| | | ACTGCAGGACGTTGTTAACCAGAACGCTCAAGCCCTGAACACTTTAGTGAAGCAACTGTCTTCTAACTTCGGCGCTAT |
| | | TTCTTCCGTTTTAAACGACATTCTGTCTCGCTTAGATCCCCCTGAGGCTGAGGTTCAAATTGATAGACTGATTACTGGA |
| | | AGATTACAGTCCCTGCAAACTTACGTTACTCAACAGTTAATCAGAGCTGCTGAAATCCGCGCTTCCGCTAATTTAGCTG |
| | | CTACTAAAATGTCTGAGTGTGTGTTAGGCCAGTCTAAGAGAGTTGACTTTTGTGGCAAGGGCTATCATTTAATGTCTTT |
| | | TCCTCAGTCCGCTCCTCACGGCGTTGTGTTCTTACACGTGACTTACGTTCCCGCCCAAGAGAAAAATTTTACCACTGC |
| | | TCCTGCTATTTGTCACGACGGAAAAGCTCATTTTCCTAGAGAAGGAGTGTTTGTTTCTAACGGAACCCACTGGTTTGTG |
| | | ACTCAAAGAAACTTTTACGAACCCCAAATTATTACTACTGACAATACTTTCGTTTCCGGCAACTGTGACGTGGTGATCG |
| | | GAATTGTTAATAACACCGTTTACGATCCCTTACAACCTGAATTAGATTCTTTTAAAGAAGAATTAGATAAATATTTCAAAA |
| | | ATCATACTTCCCCTGACGTTGATCTGGGAGATATTTCTGGAATTAACGCTTCTGTGGTGAACATTCAAAAAGAGATTGA |
| | | CAGATTAAACGAAGTTGCTAAAAACCTGAACGAGTCCCTGATCGACCTGCAGGAACTGGGAAAATACGAGCAGTATAT |
| | | TAAGTGGCCTTGGTATATTTGGTTAGGATTTATTGCTGGATTAATTGCTATTGTGATGGTGACCATTATGTTGTGCTGTA |
| | | TGACCTCTTGTTGTTCTTGCCTGAAGGGCTGCTGTTCTTGTGGCTCTTGTTGCAAGTTTGACGAAGACGATTCCGAAC |
| | | CCGTTTTAAAGGGAGTGAAACTGCATTATACTTAAATGAGACCATA |
| 248 | 2A8481 | ATAGGTCTCACATGTTTGTTTTTCTGGTTTTATTACCCTTAGTGTCCTCCCAGTGCGTGAACTTAACTACCCGCACTCA |
| | | GCTGCCTCCTGCCTATACCAACTCTTTCACTAGAGGCGTTTACTATCCTGACAAAGTTTTTAGATCTTCTGTGCTGCAT |
| | | TCCACTCAAGATTTATTCCTGCCCTTCTTTTCTAACGTTACTTGGTTTCACGCCATCCACGTGTCTGGCACCAACGGAA |
| | | CCAAACGCTTTGACAACCCTGTGTTACCTTTTAACGACGGAGTTTACTTCGCCTCCACTGAAAAGTCCAATATTATCCG |
| | | GGGCTGGATTTTCGGAACCACCTTAGATTCTAAAACCCAGTCTCTGCTGATTGTGAACAACGCCACTAACGTGGTTAT |
| | | TAAAGTGTGCGAGTTTCAATTTTGCAACGACCCTTTTCTGGGAGTTTATTACCACAAAAATAACAAATCTTGGATGGAG |
| | | TCTGAATTTAGAGTGTATTCTTCCGCCAATAATTGCACCTTCGAGTACGTTTCCCAACCCTTTCTGATGGACTTAGAAG |
| | | GAAAGCAAGGCAATTTCAAAAACCTGCGCGAATTTGTGTTCAAAAATATTGACGGCTATTTCAAAATTTACTCCAAGCA |
| | | TACCCCTATTAATTTAGTGCGCGATCTGCCTCAGGGCTTCTCTGCCTTAGAGCCTTTAGTGGACCTGCCTATTGGCAT |
| | | TAATATTACTCGCTTTCAGACTTTACTGGCCCTGCATAGATCTTATTTAACTCCTGGAGACTCTTCTTCCGGCTGGACC |
| | | GCTGGAGCCGCTGCCTACTACGTTGGCTATCTGCAGCCTAGAACTTTTTTATTAAAGTACAACGAAAACGGAACCATC |
| | | ACTGACGCTGTTGATTGCGCCCTGGATCCTTTATCTGAAACAAAGTGCACCCTGAAGTCCTTCACCGTTGAAAAGGGC |
| | | ATTTACCAAACATCCAATTTTAGGGTTCAGCCTACTGAATCCATCGTGCGCTTTCCCAACATCACTAACTTGTGTCCCT |
| | | TTGGCGAGGTGTTTAACGCCACTCGCTTTGCCTCTGTGTACGCCTGGAACAGAAAGAGAATTTCCAATTGCGTGGCTG |
| | | ATTATTCTGTGCTGTATAACTCCGCTTCTTTCTCCACCTTTAAGTGTTACGGAGTGTCCCCTACTAAACTGAACGACTT |
| | | GTGCTTCACCAACGTTTACGCCGATTCTTTTGTGATTCGCGGCGACGAAGTTAGACAAATCGCCCCCGGACAGACCG |
| | | GAAAGATCGCTGATTACAACTATAAGTTACCTGACGACTTCACCGGCTGCGTGATTGCTTGGAATTCTAATAACTTAGA |
| | | CTCTAAAGTTGGAGGCAACTACAATTACCTGTATAGATTATTCCGCAAATCTAACCTGAAACCTTTTGAGAGAGACATC |
| | | TCCACCGAAATTTACCAAGCCGGCTCTACCCCCTGCAACGGAGTTGAAGGATTTAATTGCTACTTTCCCCTGCAGTCC |
| | | TACGGATTCCAACCTACTAACGGCGTTGGATACCAACCTTACAGAGTTGTGGTGCTGTCTTTTGAATTATTACACGCCC |
| | | CTGCCACCGTTTGTGGCCCTAAGAAATCTACCAATCTGGTTAAAAACAAGTGTGTGAATTTTAATTTTAACGGCCTGAC |
| | | CGGCACTGGCGTTTTAACCGAGTCTAACAAGAAGTTTCTGCCTTTCCAACAATTTGGCAGAGACATCGCCGACACCAC |
| | | CGACGCTGTGCGCGATCCTCAAACCTTAGAAATTTTAGATATTACTCCTTGCTCTTTCGGCGGCGTTTCCGTTATTACT |
| | | CCTGGAACCAATACCTCTAATCAAGTGGCTGTGCTGTACCAGGACGTTAATTGCACTGAAGTGCCTGTGGCTATTCAC |
| | | GCCGATCAACTGACCCCCACTTGGAGAGTTTATTCTACTGGATCTAACGTGTTCCAAACTAGAGCTGGCTGTCTGATC |
| | | GGCGCCGAGCACGTTAATAATTCTTACGAGTGTGATATCCCTATTGGCGCTGGCATTTGCGCCTCTTACCAGACCCAG |
| | | ACCAACTCCCCCCGCAGAGCTAGATCCGTGGCCTCTCAGTCCATTATTGCCTATACTATGTCCTTAGGAGCCGAAAAT |
| | | TCTGTGGCTTACTCCAACAATTCTATTGCCATTCCCACTAATTTTACTATCTCTGTGACTACCGAAATCCTGCCCGTGT |
| | | CCATGACTAAGACCTCCGTGGACTGCACCATGTATATCTGCGGAGACTCTACTGAGTGTTCCAACTTATTACTGCAGT |
| | | ACGGCTCTTTCTGTACTCAGCTGAATAGAGCCCTGACTGGAATTGCTGTGGAGCAAGACAAAAATACCCAGGAGGTG |
| | | TTCGCTCAGGTGAAACAAATTTATAAGACCCCTCCCATCAAAGATTTTGGAGGCTTCAATTTTTCTCAAATTCTGCCCG |
| | | ACCCCTCCAAGCCTTCCAAAAGATCCTTTATTGAAGATCTGCTGTTCAATAAGGTTACTTTAGCCGACGCCGGCTTCAT |
| | | TAAGCAGTACGGAGATTGTCTGGGCGATATTGCTGCCCGCGACCTGATTTGCGCTCAGAAGTTCAACGGATTAACCG |
| | | TTCTGCCTCCTCTGCTGACTGACGAGATGATTGCTCAGTATACTTCTGCCCTGCTGGCTGGAACTATTACCTCTGGCT |
| | | GGACCTTCGGCGCCGGAGCTGCTCTGCAAATTCCCTTCGCCATGCAAATGGCTTACAGATTTAACGGCATCGGAGTT |
| | | ACTCAAAACGTGCTGTACGAAAACCAAAAATTAATTGCCAACCAGTTCAATTCCGCTATCGGCAAAATTCAGGATTCTC |
| | | TGTCTTCTACTGCTTCCGCTTTAGGCAAACTGCAAGACGTTGTTAACCAGAACGCCCAAGCTTTAAACACCCTGGTTAA |
| | | GCAGCTGTCCTCTAATTTTGGCGCTATCTCTTCTGTTCTGAACGACATTTTATCTAGACTGGACCCCCCTGAAGCCGA |
| | | GGTGCAGATTGATAGATTAATCACCGGAAGACTGCAATCCCTGCAGACCTACGTTACCCAGCAATTAATCAGAGCCGC |
| | | CGAGATCAGAGCCTCTGCCAACTTAGCTGCCACCAAAATGTCCGAGTGTGTGCTGGGACAGTCCAAGCGCGTGGACT |
| | | TCTGTGGAAAGGGATACCACCTGATGTCTTTTCCTCAATCTGCTCCCCACGGAGTTGTGTTTCTGCACGTTACTTACGT |
| | | GCCTGCCCAAGAGAAAAACTTTACTACCGCCCCCGCTATTTGCCACGACGGCAAGGCTCACTTCCCTAGAGAAGGAG |
| | | TTTTTGTGTCTAACGGAACCCATTGGTTTGTGACTCAAAGAAATTTTTACGAGCCTCAAATCATTACCACCGATAACACT |
| | | TTTGTTTCCGGCAATTGTGACGTGGTGATTGGAATCGTTAACAACACCGTTTACGACCCTTTACAGCCCGAATTAGACT |
| | | CTTTTAAAGAGGAGTTAGATAAATATTTTAAAAACCATACCTCTCCCGACGTGGACCTGGGAGATATTTCCGGAATTAA |
| | | CGCTTCTGTTGTGAATATCCAGAAAGAGATCGACAGATTAAACGAGGTTGCTAAAAATTTAAACGAATCCCTGATTGAC |
| | | CTGCAGGAGCTGGGCAAATACGAACAATATATTAAGTGGCCTTGGTATATTTGGTTAGGCTTCATCGCTGGATTAATC |
| | | GCTATTGTTATGGTGACTATTATGTTGTGTTGTATGACTTCCTGCTGTTCTTGCTTAAAAGGCTGCTGTTCCTGCGGAT |
| | | CCTGTTGTAAATTCGACGAGGACGATTCCGAGCCTGTTTTAAAAGGAGTGAAATTACATTATACTTAAATGAGACCATA |
| 249 | 2A9481 | ATAGGTCTCACATGTTCGTGTTCCTGGTGCTGCTGCCCTTAGTGTCCTCCCAGTGTGTGAACCTGACCACCAGAACCC |
| | | AGTTACCTCCCGCTTACACCAACTCCTTTACCCGCGGAGTGTACTACCCTGATAAGGTTTTTAGATCCTCCGTTCTGCA |
| | | TTCTACCCAAGACCTGTTTTTACCCTTCTTTTCCAACGTGACCTGGTTCCACGCTATCCACGTGTCCGGCACTAACGG |
| | | CACTAAGCGCTTCGACAATCCTGTGCTCCCTTTTAACGACGGCGTGTACTTCGCTTCTACCGAAAAATCCAACATCAT |
| | | CCGCGGCTGGATCTTCGGAACTACCCTGGATTCTAAAACCCAGTCTTTATTAATCGTGAATAACGCCACCAACGTGGT |
| | | TATTAAGGTCTGTGAGTTCCAATTTTGCAACGACCCTTTCTTAGGCGTTTATTACCACAAAAACAATAAGTCTTGGATG |
| | | GAGTCCGAATTCCGCGTGTACTCCTCTGCCAACAATTGCACCTTTGAGTACGTGTCCCAGCCCTTCCTGATGGACCTG |
| | | GAAGGCAAGCAGGGCAATTTTAAAAATCTGCGCGAATTCGTTTTCAAGAACATCGACGGCTACTTTAAGATTTATTCCA |
| | | AACATACCCCCATCAACCTGGTGCGCGATTTACCCCAGGGCTTCTCCGCCTTAGAGCCCTTAGTGGATCTGCCTATC |
| | | GGCATCAATATCACCCGCTTTCAGACTCTGCTGGCCCTGCACAGATCCTACCTGACTCCCGGAGACTCTTCTTCTGGC |
| | | TGGACCGCCGGAGCTGCCGCCTACTACGTGGGCTACCTGCAACCCCGCACCTTTCTGCTGAAGTACAACGAAAACG |
| | | GCACCATCACAGACGCCGTTGACTGCGCTCTGGACCCCCTGTCTGAGACTAAGTGCACCCTGAAGTCCTTCACTGTG |
| | | GAGAAAGGAATCTACCAGACCTCCAACTTCCGCGTGCAGCCTACCGAATCCATTGTTCGCTTCCCTAACATTACTAAT |
| | | CTGTGCCCCTTCGGAGAAGTTTTCAACGCCACTAGATTCGCTTCCGTGTACGCCTGGAATCGCAAGCGCATCTCCAAT |
| | | TGCGTTGCTGACTACTCTGTGTTATACAATTCCGCCTCTTTTTCCACTTTCAAGTGCTACGGCGTGTCTCCTACCAAGC |
| | | TGAACGACCTGTGTTTCACTAACGTGTACGCTGATTCCTTTGTTATCCGCGGCGACGAGGTGAGACAGATCGCCCCT |
| | | GGCCAGACCGGAAAAATCGCCGACTACAACTACAAGTTACCCGACGATTTTACCGGCTGTGTTATTGCTTGGAACTCT |
| | | AATAATCTGGACTCTAAAGTGGGGGGCAATTACAACTACTTATATCGCCTGTTCAGAAAATCCAACCTGAAACCCTTCG |
| | | AACGGGACATTTCCACCGAAATTTATCAGGCCGGCTCCACCCCCTGCAACGGAGTGGAGGGCTTCAATTGCTACTTC |
| | | CCCCTGCAATCCTACGGCTTTCAGCCCACCAACGGCGTGGGATACCAGCCCTACCGCGTTGTTGTGTTATCCTTTGA |
| | | ACTGCTGCACGCTCCTGCTACTGTGTGCGGACCTAAAAAATCCACCAATCTGGTGAAGAACAAGTGTGTGAATTTTAA |
| | | TTTTAACGGCCTGACCGGCACCGGAGTGCTGACCGAGTCCAACAAAAAGTTTCTGCCCTTTCAGCAGTTCGGCCGGG |
| | | ACATTGCCGACACTACCGACGCTGTGAGGGATCCTCAGACCCTGGAGATTCTGGACATTACCCCTTGCTCCTTCGGA |
| | | GGAGTTTCCGTGATCACCCCTGGCACCAATACTTCTAACCAAGTGGCTGTTTTATACCAGGACGTGAATTGTACTGAG |
| | | GTGCCTGTGGCCATCCACGCCGATCAATTAACTCCCACCTGGAGAGTGTATTCCACCGGCTCTAACGTTTTCCAAACC |
| | | CGCGCTGGCTGCTTAATCGGCGCTGAGCACGTGAACAACTCCTACGAGTGTGATATTCCTATCGGAGCCGGCATCTG |
| | | TGCTTCCTACCAGACTCAGACCAACTCCCCCAGACGCGCTAGGTCCGTGGCTTCCCAGTCTATTATCGCTTATACTAT |
| | | GTCCCTGGGAGCCGAGAATTCTGTTGCCTATTCCAATAACTCCATCGCCATTCCTACCAACTTTACCATCTCTGTGACC |
| | | ACCGAGATTCTGCCCGTGTCCATGACCAAAACTTCTGTGGATTGTACCATGTACATCTGTGGAGATTCCACCGAGTGC |
| | | TCTAACCTGCTGCTGCAGTACGGCTCCTTTTGCACCCAATTAAACCGCGCTCTGACTGGCATCGCCGTGGAGCAAGA |
| | | TAAAAACACCCAAGAGGTGTTCGCTCAGGTGAAGCAGATCTACAAGACACCTCCCATTAAAGATTTCGGAGGCTTTAA |
| | | TTTCTCTCAGATCCTGCCCGACCCTTCTAAGCCCTCTAAACGCTCCTTCATCGAGGATCTGCTGTTCAACAAGGTTAC |
| | | CCTGGCCGACGCTGGCTTTATCAAACAGTACGGCGACTGCTTAGGCGACATCGCCGCCCGCGATTTAATTTGCGCTC |
| | | AGAAGTTCAACGGACTGACCGTGCTGCCTCCTCTGCTGACCGACGAAATGATCGCCCAATACACCTCCGCCCTGCTG |
| | | GCTGGCACTATCACCTCCGGCTGGACCTTTGGCGCTGGAGCCGCTCTGCAAATCCCTTTCGCTATGCAGATGGCTTA |
| | | CCGCTTTAACGGAATCGGCGTGACCCAGAACGTGTTATACGAAAATCAGAAATTAATTGCCAATCAATTTAACTCTGCC |
| | | ATCGGCAAGATTCAGGATTCTCTGTCTTCTACTGCCTCTGCCTTAGGAAAACTGCAGGACGTGGTGAATCAGAACGCC |
| | | CAGGCCCTGAATACCCTGGTTAAGCAACTGTCTTCCAATTTCGGCGCTATCTCCTCTGTGTTAAACGACATTCTGTCC |
| | | CGCCTGGATCCTCCTGAGGCCGAAGTGCAAATTGACCGCTTAATCACTGGCAGACTGCAATCCCTGCAAACCTACGT |
| | | TACTCAACAGCTGATCAGAGCCGCCGAAATCCGCGCTTCTGCCAACTTAGCTGCTACCAAGATGTCCGAGTGCGTGC |
| | | TGGGACAGTCCAAAAGAGTTGACTTTTGCGGAAAGGGCTACCACCTGATGTCCTTTCCCCAGTCCGCCCCCCACGGA |
| | | GTTGTTTTCCTGCACGTTACTTACGTTCCTGCTCAAGAGAAGAACTTCACCACCGCTCCCGCTATTTGCCACGACGGC |
| | | AAGGCTCATTTCCCCAGAGAGGGAGTGTTTGTGTCCAACGGCACTCACTGGTTTGTTACTCAGCGCAACTTCTACGAA |
| | | CCTCAGATCATCACCACTGACAATACCTTTGTGTCCGGAAACTGTGACGTGGTTATCGGCATTGTGAACAATACTGTG |
| | | TACGACCCTTTACAACCTGAGCTGGATTCTTTTAAAGAAGAACTGGACAAGTACTTCAAGAACCACACCTCCCCCGAC |
| | | GTGGATCTGGGCGACATCTCCGGCATCAACGCCTCTGTGGTGAATATTCAGAAGGAGATTGATCGCCTGAACGAGGT |
| | | TGCCAAGAATCTGAACGAGTCTCTGATCGACCTGCAGGAGCTGGGCAAGTACGAACAGTACATCAAGTGGCCCTGGT |
| | | ACATTTGGCTGGGCTTTATTGCTGGACTGATCGCTATCGTTATGGTTACCATCATGCTGTGCTGTATGACCTCTTGCTG |
| | | CTCCTGTCTGAAAGGCTGTTGCTCCTGCGGCTCCTGTTGTAAGTTTGACGAGGACGACTCCGAGCCTGTTCTGAAAG |
| | | GAGTTAAACTGCACTACACCTAAATGAGACCATA |
| 250 | 2A0581 | ATAGGTCTCACATGTTTGTGTTTCTGGTGCTGCTGCCTCTGGTGTCCTCTCAGTGTGTTAACCTGACCACCAGAACCC |
| | | AACTGCCCCCCGCCTACACTAACTCTTTCACTCGCGGCGTGTACTACCCCGATAAAGTTTTCAGATCCTCCGTGCTCC |
| | | ATTCTACCCAAGACCTGTTCTTACCCTTTTTCTCCAACGTGACCTGGTTCCACGCCATCCACGTGTCCGGCACTAACG |
| | | GAACCAAGCGCTTCGATAATCCCGTGCTGCCTTTCAACGACGGAGTTTACTTTGCTTCCACCGAGAAGTCCAATATTA |
| | | TCCGGGGCTGGATCTTCGGCACCACACTGGATTCCAAAACCCAGTCCTTACTGATTGTGAACAACGCCACTAACGTG |
| | | GTGATCAAGGTGTGTGAGTTCCAGTTCTGTAACGACCCCTTTCTGGGCGTGTATTATCATAAAAACAACAAGTCCTGG |
| | | ATGGAGTCCGAGTTTAGAGTGTATTCTTCCGCTAACAACTGCACCTTCGAATACGTGTCCCAGCCTTTCCTGATGGAT |
| | | CTGGAAGGAAAGCAGGGCAACTTCAAGAACTTACGCGAGTTCGTGTTCAAGAACATCGACGGCTACTTCAAAATTTAC |
| | | TCCAAGCACACCCCTATCAACTTAGTGAGAGATCTGCCCCAGGGCTTCTCCGCTCTGGAACCTCTGGTGGACTTACC |
| | | CATTGGCATCAACATCACTCGCTTCCAAACACTGCTCGCTCTGCACCGCTCTTATCTGACCCCCGGCGACTCCTCTTC |
| | | TGGCTGGACCGCCGGCGCCGCTGCCTATTACGTGGGATACCTGCAACCCCGCACCTTTCTGCTGAAATACAACGAAA |
| | | ACGGCACCATCACAGACGCTGTGGACTGCGCTCTGGACCCCCTGTCCGAGACTAAGTGCACCCTGAAGTCCTTCACC |
| | | GTTGAAAAGGGCATTTATCAGACTTCCAACTTCCGCGTGCAGCCTACTGAGTCCATTGTGCGCTTCCCTAACATCACT |
| | | AACTTGTGTCCTTTCGGCGAAGTGTTCAACGCTACTCGCTTCGCCTCCGTGTACGCCTGGAACCGCAAGAGAATCTC |
| | | CAATTGCGTGGCCGACTACTCTGTTCTCTACAACTCTGCCTCCTTCTCCACCTTTAAGTGCTACGGCGTGTCCCCCAC |
| | | CAAACTGAACGACCTGTGCTTCACCAACGTGTACGCCGACTCCTTTGTGATCAGAGGCGACGAGGTGCGCCAGATCG |
| | | CCCCCGGCCAGACTGGCAAGATCGCCGATTACAATTACAAGCTGCCTGACGACTTTACCGGCTGCGTGATTGCCTGG |
| | | AACTCCAACAACCTGGACTCCAAAGTGGGGGGCAACTACAACTACCTGTACCGCCTGTTTAGAAAATCCAACCTGAAG |
| | | CCCTTCGAGCGGGACATTTCTACTGAGATCTACCAGGCCGGCTCCACCCCCTGTAACGGCGTGGAAGGCTTCAACTG |
| | | CTACTTTCCCCTGCAGTCCTACGGCTTCCAGCCTACCAACGGCGTGGGCTACCAGCCCTACCGCGTGGTGGTGTTAT |
| | | CCTTCGAGCTGCTGCACGCCCCTGCCACCGTGTGCGGACCCAAAAAGTCTACTAACCTGGTTAAGAATAAGTGTGTT |
| | | AATTTCAATTTTAACGGCTTAACCGGAACCGGAGTGCTGACCGAGTCCAATAAAAAGTTCCTGCCCTTTCAGCAGTTT |
| | | GGCCGCGACATCGCCGATACTACCGACGCCGTGCGCGATCCCCAGACCCTGGAGATCCTGGACATTACCCCCTGCT |
| | | CTTTCGGCGGAGTGTCCGTGATCACCCCCGGCACCAATACCTCCAATCAGGTGGCCGTTCTGTACCAGGACGTGAAT |
| | | TGCACCGAAGTGCCCGTGGCCATTCACGCCGACCAGTTAACTCCTACTTGGAGAGTGTACTCCACCGGCTCTAACGT |
| | | GTTTCAGACCCGCGCCGGCTGTCTGATCGGCGCCGAGCACGTGAACAACTCCTACGAGTGCGACATTCCCATTGGC |
| | | GCCGGCATCTGCGCCTCCTACCAAACCCAGACTAACTCTCCCAGACGCGCCCGCTCTGTGGCCTCTCAGTCCATCAT |
| | | CGCTTACACTATGTCCCTGGGCGCCGAGAACTCCGTGGCCTATTCCAACAACTCCATCGCCATCCCCACTAACTTCAC |
| | | CATCTCTGTGACCACCGAAATCCTCCCCGTGTCTATGACTAAGACTTCCGTGGACTGTACCATGTACATTTGCGGCGA |
| | | CTCCACCGAGTGTTCCAATCTGCTGCTGCAGTACGGCTCCTTTTGCACCCAGCTGAACCGCGCCCTGACCGGCATCG |
| | | CTGTTGAGCAGGACAAGAATACCCAGGAGGTGTTTGCTCAGGTGAAGCAAATCTATAAGACCCCTCCCATCAAGGAC |
| | | TTTGGCGGCTTCAACTTTTCCCAGATCCTGCCCGACCCTTCCAAGCCCTCCAAACGCTCTTTCATCGAAGATCTGCTG |
| | | TTCAATAAAGTGACCCTGGCCGACGCCGGCTTCATCAAGCAGTACGGAGACTGTCTGGGAGACATTGCTGCTCGCGA |
| | | CCTGATTTGCGCCCAGAAGTTCAACGGCCTGACCGTGCTGCCCCCTCTGCTGACCGACGAGATGATCGCTCAGTACA |
| | | CCTCCGCCCTGCTGGCTGGCACTATCACCTCCGGCTGGACTTTCGGCGCTGGCGCCGCCCTGCAGATTCCCTTCGC |
| | | CATGCAGATGGCTTATCGCTTCAACGGAATCGGAGTGACACAGAACGTGCTGTACGAGAATCAGAAACTGATCGCTA |
| | | ACCAGTTTAATTCCGCCATTGGCAAGATCCAGGACTCCCTGTCCTCTACTGCTTCCGCCCTGGGCAAGCTGCAGGAC |
| | | GTGGTGAACCAGAACGCCCAGGCTCTGAACACCCTGGTGAAGCAACTGTCTTCTAACTTTGGAGCCATTTCTTCCGTG |
| | | CTGAACGACATCCTGTCCAGACTGGATCCTCCTGAGGCCGAGGTGCAGATCGACCGCCTGATCACTGGCCGCCTGC |
| | | AGTCCCTGCAGACTTACGTTACCCAGCAATTAATCCGCGCTGCCGAGATCCGCGCCTCCGCCAACCTGGCCGCCACC |
| | | AAGATGTCCGAGTGCGTTCTGGGCCAGTCCAAACGCGTTGACTTCTGCGGCAAGGGCTACCACCTGATGTCCTTCCC |
| | | CCAGTCCGCTCCCCACGGAGTGGTGTTCCTGCACGTGACCTACGTGCCTGCCCAGGAGAAGAACTTCACCACCGCC |
| | | CCCGCCATTTGCCACGACGGAAAGGCCCACTTCCCCCGCGAGGGAGTGTTCGTTTCTAACGGCACTCACTGGTTCGT |
| | | GACCCAGAGAAACTTCTACGAGCCCCAGATTATTACCACCGACAATACCTTCGTTTCTGGCAACTGCGACGTGGTTAT |
| | | CGGCATCGTGAACAACACCGTGTACGACCCCCTGCAACCCGAGCTGGATTCTTTCAAGGAGGAGCTGGATAAGTACT |
| | | TTAAAAACCATACCTCCCCCGACGTGGACCTGGGCGACATCTCTGGCATCAACGCCTCCGTGGTGAATATCCAGAAG |
| | | GAGATCGATCGCCTGAACGAGGTGGCCAAGAACTTAAACGAATCTCTGATCGACCTGCAGGAGCTGGGCAAGTACGA |
| | | GCAGTATATTAAGTGGCCCTGGTATATCTGGTTAGGCTTTATCGCCGGCCTGATCGCCATTGTTATGGTGACCATCAT |
| | | GCTGTGCTGCATGACCTCCTGCTGTTCCTGTTTAAAAGGCTGCTGCTCCTGCGGATCCTGCTGTAAATTCGACGAAGA |
| | | CGACTCCGAGCCCGTGCTGAAAGGAGTTAAGTTACACTATACCTAAATGAGACCATA |
| 251 | 2A1581 | ATAGGTCTCACATGTTCGTGTTCCTGGTGCTGCTGCCCCTGGTGTCCTCCCAGTGCGTCAACCTGACCACCCGGACC |
| | | CAACTGCCCCCTGCCTATACCAATTCCTTCACCCGGGGCGTCTACTACCCCGACAAGGTGTTCCGGTCCTCTGTGCT |
| | | CCACTCCACCCAGGACCTCTTCCTCCCCTTTTTCTCCAACGTGACCTGGTTCCACGCCATCCACGTGTCTGGCACAAA |
| | | CGGAACCAAGCGCTTCGACAACCCTGTGCTCCCCTTCAACGACGGCGTGTACTTCGCCTCCACCGAGAAGTCCAACA |
| | | TCATCCGCGGCTGGATCTTCGGCACCACCCTCGACTCTAAGACCCAGTCCCTGCTGATTGTGAATAACGCCACCAAC |
| | | GTGGTGATCAAGGTGTGCGAATTTCAGTTCTGCAACGACCCCTTCCTGGGCGTTTACTACCATAAGAACAACAAGTCC |
| | | TGGATGGAGTCCGAGTTCCGGGTGTATTCCTCTGCCAATAACTGCACCTTTGAGTACGTGTCCCAGCCCTTCCTGATG |
| | | GACCTGGAGGGCAAGCAGGGCAATTTTAAAAACCTGCGCGAGTTCGTGTTCAAGAACATCGACGGCTACTTCAAGAT |
| | | CTATTCCAAGCACACCCCTATCAACCTGGTGCGCGACCTGCCCCAGGGCTTTTCCGCTCTGGAGCCCCTGGTTGACC |
| | | TGCCCATCGGCATCAACATCACCCGCTTCCAGACTTTACTGGCCCTGCACCGCTCCTACCTGACCCCCGGCGATTCC |
| | | TCTTCTGGCTGGACAGCCGGAGCCGCTGCCTACTACGTGGGATACCTGCAGCCCCGCACCTTCCTGCTGAAGTACAA |
| | | CGAGAACGGAACCATTACAGACGCTGTCGACTGTGCTCTGGATCCCCTGTCCGAAACAAAGTGCACCCTGAAGTCCT |
| | | TCACCGTGGAGAAGGGCATCTACCAGACCTCCAACTTCCGCGTGCAGCCCACCGAGTCCATCGTGAGATTCCCCAAC |
| | | ATCACTAACCTGTGCCCCTTCGGCGAGGTGTTCAACGCCACCCGCTTCGCCTCCGTGTACGCCTGGAACCGCAAGC |
| | | GCATCTCCAACTGCGTTGCCGACTACTCTGTGCTCTACAACTCCGCCTCCTTCTCCACCTTCAAGTGCTACGGCGTGT |
| | | CCCCCACCAAGCTGAACGACCTGTGCTTCACCAACGTGTACGCCGACTCCTTCGTGATCCGCGGCGACGAGGTGCG |
| | | CCAGATCGCCCCCGGCCAGACCGGAAAGATCGCCGATTACAATTACAAGCTGCCCGACGACTTCACCGGCTGCGTG |
| | | ATCGCCTGGAACTCCAACAATCTGGACTCCAAGGTTGGCGGCAACTACAACTACCTGTACCGCCTGTTCCGCAAGTC |
| | | CAACCTGAAGCCCTTCGAGCGGGACATTTCCACAGAGATTTACCAGGCTGGCTCCACCCCCTGTAACGGAGTGGAGG |
| | | GCTTCAACTGCTACTTCCCCCTGCAGTCCTACGGCTTCCAGCCCACCAACGGCGTGGGCTACCAGCCCTACCGCGTG |
| | | GTGGTGCTGTCCTTCGAGCTGCTGCACGCCCCTGCCACCGTGTGCGGCCCCAAGAAGTCCACCAACCTGGTGAAGA |
| | | ACAAGTGCGTGAACTTCAACTTCAACGGACTGACAGGCACCGGCGTGCTGACCGAGTCCAACAAGAAGTTCCTGCCC |
| | | TTCCAGCAGTTCGGCCGGGACATTGCCGACACCACCGACGCCGTGCGCGACCCCCAGACCCTGGAGATCCTGGACA |
| | | TCACCCCCTGCTCCTTCGGCGGAGTGTCCGTGATCACCCCCGGCACCAACACCTCCAACCAGGTTGCCGTGCTGTAC |
| | | CAGGACGTTAACTGCACCGAGGTGCCCGTGGCCATCCACGCCGACCAGCTGACCCCCACCTGGCGCGTGTACTCCA |
| | | CCGGCTCCAACGTGTTCCAGACCCGCGCCGGCTGCCTGATCGGCGCCGAGCACGTGAACAACTCCTACGAGTGCGA |
| | | CATCCCCATTGGCGCCGGCATCTGCGCCTCCTACCAGACCCAGACCAACTCCCCTCGCCGCGCCCGGTCCGTCGCC |
| | | TCCCAGTCCATCATCGCCTACACCATGTCCCTGGGCGCCGAGAACTCCGTGGCCTACTCCAACAACTCCATTGCCAT |
| | | CCCCACCAACTTCACCATCTCCGTGACCACCGAAATCCTCCCCGTGTCCATGACCAAGACCTCCGTGGACTGCACCA |
| | | TGTACATCTGCGGCGACTCCACCGAGTGCTCCAACCTGCTGCTGCAGTACGGCTCCTTCTGCACCCAGCTGAATCGC |
| | | GCCCTGACCGGCATCGCCGTGGAGCAGGACAAGAACACCCAAGAGGTGTTCGCCCAGGTGAAGCAGATCTACAAGA |
| | | CACCTCCCATCAAGGACTTCGGCGGCTTCAACTTCTCCCAGATCCTGCCCGACCCCTCCAAGCCCTCCAAGCGCTCC |
| | | TTCATCGAGGACCTGCTGTTCAACAAGGTGACCCTGGCCGACGCCGGCTTCATCAAGCAGTACGGCGACTGTCTGG |
| | | GAGACATTGCCGCCCGCGACCTGATCTGCGCCCAGAAGTTCAACGGCTTAACCGTGCTGCCCCCTCTGCTGACCGA |
| | | CGAGATGATCGCCCAGTACACATCCGCTCTGCTGGCCGGCACCATCACCTCCGGCTGGACCTTTGGCGCTGGCGCT |
| | | GCCCTGCAGATCCCCTTCGCTATGCAGATGGCCTACCGCTTCAACGGCATCGGCGTGACCCAGAACGTGCTGTACGA |
| | | GAACCAGAAGCTGATCGCCAACCAGTTCAACTCCGCCATCGGCAAGATCCAGGACTCCCTGTCCTCCACCGCCTCCG |
| | | CCCTGGGCAAGCTGCAGGACGTGGTGAACCAGAACGCCCAGGCCCTGAACACCCTGGTGAAGCAGCTGTCCTCTAA |
| | | CTTCGGCGCCATCTCCTCCGTGCTGAACGACATTCTGTCCCGCCTGGATCCTCCTGAGGCCGAGGTGCAAATCGATC |
| | | GCCTCATCACCGGCCGCCTGCAGTCCCTGCAGACCTACGTGACACAGCAGCTGATCCGCGCCGCCGAGATCCGCGC |
| | | CTCCGCCAACCTGGCCGCCACCAAGATGTCTGAGTGCGTGCTGGGCCAGTCCAAACGCGTTGACTTCTGCGGCAAG |
| | | GGCTACCACCTGATGTCCTTCCCCCAGTCCGCCCCCCACGGCGTGGTGTTCCTGCACGTGACCTACGTGCCCGCCC |
| | | AGGAGAAGAACTTCACTACCGCCCCCGCCATCTGCCACGACGGCAAGGCCCACTTCCCCCGCGAGGGCGTGTTCGT |
| | | GTCCAACGGCACCCACTGGTTCGTGACCCAGCGCAACTTCTACGAGCCCCAGATCATCACCACCGACAACACCTTCG |
| | | TGTCCGGCAACTGCGACGTGGTGATTGGCATCGTGAACAACACCGTGTACGACCCCCTGCAGCCCGAGCTGGACTC |
| | | CTTCAAGGAGGAGCTGGACAAGTACTTTAAGAACCACACCTCCCCCGACGTGGACCTGGGCGACATCTCCGGCATCA |
| | | ACGCCTCCGTGGTGAACATCCAGAAGGAGATCGACCGCCTGAACGAGGTGGCCAAGAACCTGAACGAGTCCCTGAT |
| | | CGACCTGCAGGAGCTGGGCAAGTACGAACAGTACATCAAGTGGCCCTGGTACATCTGGCTGGGCTTCATCGCCGGC |
| | | CTGATCGCCATCGTGATGGTGACCATCATGCTGTGCTGCATGACCTCTTGCTGTTCCTGTCTGAAGGGCTGCTGCTC |
| | | CTGCGGCTCCTGCTGCAAGTTCGACGAGGACGACTCCGAGCCCGTGCTGAAGGGCGTGAAGCTGCACTACACCTAA |
| | | ATGAGACCATA |
| 252 | 2A2581 | ATAGGTCTCACATGTTCGTGTTCCTGGTGCTGCTCCCCCTGGTGAGCTCCCAGTGCGTCAACCTGACCACACGGACC |
| | | CAGCTGCCCCCCGCCTATACCAACAGCTTCACCCGCGGCGTCTACTACCCCGACAAGGTGTTCCGGAGCAGCGTGC |
| | | TGCACAGCACCCAGGACCTCTTCCTCCCCTTTTTTAGCAACGTGACCTGGTTCCACGCCATCCACGTGAGTGGCACC |
| | | AACGGCACCAAGCGGTTCGACAACCCTGTGCTCCCCTTCAACGACGGCGTGTACTTCGCTAGCACAGAAAAAAGTAA |
| | | CATCATTCGGGGCTGGATCTTCGGCACCACACTGGATAGCAAAACCCAGAGCCTGCTGATTGTGAATAACGCCACCA |
| | | ACGTGGTGATCAAGGTGTGCGAGTTCCAGTTCTGCAACGACCCCTTCCTGGGCGTGTACTACCACAAGAACAACAAG |
| | | AGCTGGATGGAGAGCGAGTTCCGCGTGTATAGCTCTGCCAACAACTGCACCTTCGAGTACGTGAGCCAGCCCTTCCT |
| | | GATGGACCTGGAGGGCAAGCAGGGCAATTTCAAAAACCTGCGGGAGTTCGTGTTCAAGAACATCGACGGCTACTTCA |
| | | AGATCTACAGCAAGCACACCCCCATCAACCTGGTGCGGGACCTGCCCCAGGGCTTTAGCGCTCTGGAGCCCCTGGT |
| | | GGACCTGCCCATCGGCATCAACATCACCCGCTTCCAAACCCTGTTGGCCCTGCACCGGAGCTACCTGACCCCTGGC |
| | | GATAGCAGTAGTGGCTGGACAGCCGGAGCCGCCGCCTACTACGTGGGATACCTGCAGCCCCGGACCTTCCTGCTGA |
| | | AGTACAACGAGAACGGCACAATCACAGACGCCGTGGACTGCGCCCTGGACCCCCTGAGCGAAACAAAGTGCACCCT |
| | | CAAAAGTTTCACCGTGGAGAAGGGCATCTACCAGACCAGCAACTTCCGGGTGCAGCCCACCGAGAGCATCGTGCGG |
| | | TTCCCCAACATCACCAACCTGTGCCCCTTCGGCGAGGTGTTCAACGCCACCCGGTTCGCCAGCGTGTACGCCTGGAA |
| | | CCGGAAGCGGATCAGCAACTGCGTGGCCGACTACAGCGTGCTGTATAACAGTGCCAGCTTCAGCACCTTCAAGTGCT |
| | | ACGGCGTGAGCCCCACCAAGCTGAACGACCTGTGCTTCACCAACGTGTACGCCGACAGCTTCGTGATCCGGGGCGA |
| | | CGAGGTGCGGCAGATCGCCCCCGGCCAGACCGGCAAGATCGCCGATTACAATTACAAGCTGCCCGACGATTTCACC |
| | | GGCTGCGTGATCGCCTGGAACAGCAACAACCTGGACAGCAAGGTGGGGGGCAACTACAACTACCTGTACCGGCTGT |
| | | TCCGCAAAAGTAACCTGAAGCCCTTCGAGCGGGACATCAGCACCGAGATCTACCAGGCCGGCAGTACCCCTTGCAAC |
| | | GGCGTCGAGGGCTTCAACTGCTACTTCCCCCTGCAGAGCTACGGCTTCCAGCCCACCAACGGCGTGGGCTACCAGC |
| | | CCTACCGGGTGGTGGTTCTGAGCTTCGAGCTGCTGCACGCCCCTGCCACCGTGTGCGGCCCCAAGAAAAGCACCAA |
| | | CCTGGTGAAGAACAAGTGCGTGAATTTCAATTTTAACGGACTCACCGGAACCGGCGTGCTGACCGAGAGCAACAAGA |
| | | AGTTCCTGCCCTTCCAGCAATTCGGCCGGGATATTGCCGACACCACTGACGCCGTGCGGGACCCCCAGACCCTGGA |
| | | GATCCTGGACATCACACCCTGTAGCTTCGGCGGGGTGAGCGTGATCACCCCCGGCACCAACACCAGCAACCAGGTG |
| | | GCCGTGCTGTACCAGGACGTGAACTGCACCGAGGTGCCCGTGGCCATCCACGCCGACCAGCTGACCCCCACCTGG |
| | | CGGGTGTACAGCACCGGCAGCAACGTGTTCCAGACCCGGGCCGGCTGCCTGATCGGCGCCGAGCACGTGAATAAC |
| | | AGCTACGAGTGCGACATCCCCATCGGAGCCGGAATCTGCGCCAGCTACCAGACCCAGACCAACAGCCCCCGGAGAG |
| | | CCCGGAGCGTGGCCAGCCAGAGCATTATTGCCTACACCATGAGCCTGGGCGCCGAGAACAGCGTGGCCTACAGCAA |
| | | CAATAGTATTGCCATCCCCACCAACTTCACCATCAGCGTGACCACCGAAATCCTCCCCGTGAGCATGACCAAGACCA |
| | | GCGTGGACTGCACCATGTACATCTGCGGCGACAGCACCGAGTGCAGCAACCTGCTCCTGCAGTACGGCAGCTTCTG |
| | | CACCCAGCTGAACCGGGCCCTGACCGGAATTGCCGTGGAGCAGGACAAGAACACCCAGGAGGTGTTCGCCCAGGT |
| | | GAAGCAGATCTACAAGACACCTCCCATCAAGGACTTCGGCGGCTTCAACTTCAGCCAGATCCTGCCCGACCCCAGCA |
| | | AGCCCAGCAAGCGGAGCTTCATCGAGGACCTGTTGTTCAACAAGGTGACCCTGGCCGACGCCGGCTTCATCAAGCA |
| | | GTACGGCGACTGTCTGGGAGATATTGCCGCACGGGACCTGATCTGCGCCCAGAAGTTCAACGGCCTGACCGTGCTG |
| | | CCCCCACTGCTGACCGACGAGATGATCGCCCAGTACACAAGCGCTCTGCTCGCCGGCACCATCACCAGCGGCTGGA |
| | | CCTTCGGCGCCGGCGCTGCCCTGCAGATCCCCTTCGCCATGCAGATGGCCTACCGGTTCAACGGCATCGGCGTGAC |
| | | CCAGAACGTGCTGTACGAGAACCAGAAGCTGATCGCCAACCAGTTCAACAGCGCCATCGGCAAGATCCAGGACAGC |
| | | CTGAGCTCCACCGCCAGCGCCCTGGGCAAGCTGCAGGACGTGGTGAACCAGAACGCCCAGGCCCTGAACACCCTG |
| | | GTGAAGCAGCTGAGCAGCAACTTCGGCGCCATCAGCTCTGTGCTGAACGACATCCTGAGCCGGCTGGATCCTCCTG |
| | | AGGCCGAGGTGCAAATCGATCGGCTCATCACCGGCCGGCTGCAGAGCCTGCAGACCTACGTGACACAGCAGCTGAT |
| | | CCGGGCCGCCGAGATCCGGGCCAGCGCCAACCTGGCCGCTACCAAGATGAGCGAGTGCGTGCTGGGCCAGAGCAA |
| | | GCGGGTGGACTTCTGCGGCAAGGGCTACCACCTGATGAGCTTCCCCCAGAGCGCCCCCCACGGCGTGGTGTTCCTG |
| | | CACGTGACCTACGTGCCCGCCCAGGAGAAGAACTTCACCACCGCCCCCGCCATCTGCCACGACGGCAAGGCCCACT |
| | | TCCCCCGGGAGGGCGTGTTCGTGAGCAACGGCACCCACTGGTTCGTGACCCAGCGGAACTTCTACGAGCCCCAGAT |
| | | CATAACCACAGACAACACCTTCGTGAGCGGCAACTGCGACGTGGTCATCGGCATCGTGAACAACACCGTGTACGACC |
| | | CCCTGCAGCCCGAGCTGGACAGCTTCAAGGAGGAACTGGACAAGTACTTCAAGAACCACACCAGCCCCGACGTGGA |
| | | CCTGGGCGACATCAGCGGCATCAACGCCAGCGTGGTGAACATCCAGAAGGAGATCGACCGGCTGAACGAGGTGGC |
| | | CAAGAACCTGAACGAGAGCCTGATCGACCTGCAGGAGCTGGGCAAGTACGAGCAGTACATCAAGTGGCCCTGGTAC |
| | | ATCTGGCTGGGCTTTATTGCCGGCCTGATCGCCATCGTGATGGTGACCATCATGCTGTGCTGTATGACAAGCTGTTG |
| | | CAGTTGCCTGAAGGGCTGCTGCAGCTGCGGCAGCTGCTGCAAGTTCGACGAGGACGACAGCGAGCCCGTGCTGAA |
| | | GGGCGTGAAGCTGCACTACACCTAAATGAGACCATA |
| 253 | 2A3581 | ATAGGTCTCACACATTAAAACAGCCTGTGGGTTGATCCCACCCACAGGCCCATTGGGCGCTAGCACTCTGGTATCAC |
| | | GGTACCTTTGTGCGCCTGTTTTATACCCCCTCCCCCAACTGTAACTTAGAAGTAACACACACCGATCAACAGTCAGCG |
| | | TGGCACACCAGCCACGTTTTGATCAAGCACTTCTGTTACCCCGGACTGAGTATCAATAGACTGCTCACGCGGTTGAAG |
| | | GAGAAAGCGTTCGTTATCCGGCCAACTACTTCGAAAAACCTAGTAACACCGTGGAAGTTGCAGAGTGTTTCGCTCAGC |
| | | ACTACCCCAGTGTAGATCAGGTCGATGAGTCACCGCATTCCCCACGGGCGACCGTGGCGGTGGCTGCGTTGGCGGC |
| | | CTGCCCATGGGGAAACCCATGGGACGCTCTAATACAGACATGGTGCGAAGAGTCTATTGAGCTAGTTGGTAGTCCTC |
| | | CGGCCCCTGAATGCGGCTAATCCTAACTGCGGAGCACACACCCTCAAGCCAGAGGGCAGTGTGTCGTAACGGGCAA |
| | | CTCTGCAGCGGAACCGACTACTTTGGGTGTCCGTGTTTCATTTTATTCCTATACTGGCTGCTTATGGTGACAATTGAGA |
| | | GATCGTTACCATATAGCTATTGGATTGGCCATCCGGTGACTAATAGAGCTATTATATATCCCTTTGTTGGGTTTATACC |
| | | ACTTAGCTTGAAAGAGGTTAAAACATTACAATTCATTGTTAAGTTGAATACAGCAACATGTTTGTTTTCTTAGTTTTATTA |
| | | CCTTTAGTTTCTTCCCAGTGTGTTAATTTAACTACTAGAACTCAATTACCTCCTGCTTATACTAATTCTTTTACTAGAGGA |
| | | GTGTATTATCCTGATAAGGTTTTTAGATCTTCTGTTCTGCACTCTACTCAGGATTTGTTCTTGCCCTTCTTCTCTAACGT |
| | | GACCTGGTTCCACGCTATTCACGTTTCTGGAACCAACGGCACTAAGAGATTTGATAATCCCGTTTTGCCTTTTAACGAC |
| | | GGAGTTTATTTCGCCTCTACCGAAAAATCTAACATCATTAGGGGCTGGATCTTTGGAACTACCCTGGATTCTAAAACTC |
| | | AATCTTTATTGATTGTGAATAACGCTACCAACGTTGTGATTAAAGTTTGTGAATTTCAATTTTGTAACGATCCTTTTCTG |
| | | GGAGTTTATTATCATAAAAACAATAAATCTTGGATGGAATCTGAATTCCGCGTTTATTCTTCTGCCAATAACTGTACCTT |
| | | TGAGTACGTTTCTCAACCTTTCTTAATGGATTTAGAAGGAAAACAAGGCAATTTCAAAAACTTAAGAGAATTTGTTTTTA |
| | | AAAATATTGACGGATATTTTAAAATTTATTCTAAGCATACTCCTATTAATTTAGTGAGAGACTTACCCCAAGGATTTTCTG |
| | | CTTTAGAACCTTTAGTTGACTTACCTATTGGAATCAATATCACTAGATTTCAAACTTTATTAGCTTTACATAGATCTTATT |
| | | TAACTCCTGGAGATTCTTCCTCCGGCTGGACAGCCGGAGCCGCTGCTTATTACGTTGGATATTTACAGCCTAGAACTT |
| | | TTCTGCTGAAATATAACGAAAACGGAACCATTACCGACGCCGTTGACTGTGCTCTGGATCCTTTATCTGAAACCAAGT |
| | | GTACTTTAAAATCTTTTACCGTTGAAAAAGGAATTTATCAAACTTCTAATTTTAGAGTTCAACCTACTGAATCTATCGTTA |
| | | GATTCCCTAATATTACTAATTTGTGTCCCTTTGGAGAGGTTTTCAACGCTACTAGATTTGCCTCTGTTTACGCTTGGAAT |
| | | CGCAAGAGAATTTCTAATTGTGTGGCCGATTACTCCGTTTTGTATAACTCTGCTTCTTTCTCTACTTTTAAGTGTTACGG |
| | | AGTTTCCCCTACTAAACTGAACGATTTGTGTTTTACTAACGTTTACGCTGATTCTTTTGTTATTAGAGGCGACGAAGTTA |
| | | GACAAATTGCTCCTGGACAAACTGGAAAGATTGCTGACTATAACTATAAACTGCCTGACGATTTTACCGGCTGTGTTAT |
| | | TGCTTGGAATTCTAATAATTTAGATTCTAAAGTTGGAGGAAATTATAATTATTTATATAGATTATTTAGAAAATCTAATTTA |
| | | AAACCTTTTGAAAGAGATATTTCCACTGAGATTTACCAGGCTGGATCTACTCCTTGTAACGGCGTTGAGGGATTTAATT |
| | | GTTATTTTCCTTTACAATCTTACGGATTTCAACCTACCAACGGAGTTGGATATCAACCTTACAGAGTTGTGGTGCTGTC |
| | | TTTTGAATTATTACACGCTCCCGCTACTGTTTGCGGCCCTAAAAAGTCTACTAATCTGGTTAAGAATAAGTGTGTGAAT |
| | | TTTAATTTCAACGGATTGACCGGAACTGGAGTTCTGACTGAATCTAATAAAAAATTTTTACCCTTTCAACAATTTGGAAG |
| | | GGATATCGCTGATACTACTGACGCTGTTAGAGATCCTCAAACTTTAGAAATCCTGGATATTACTCCTTGTTCTTTTGGA |
| | | GGAGTTTCCGTTATTACTCCTGGCACTAATACTTCTAATCAAGTTGCTGTTTTATATCAAGACGTTAATTGCACCGAAGT |
| | | TCCTGTTGCTATTCACGCTGATCAGTTAACTCCTACTTGGAGAGTTTATTCTACTGGATCTAACGTTTTTCAAACCAGA |
| | | GCTGGCTGTTTAATCGGAGCTGAACACGTGAATAATTCTTACGAGTGCGATATTCCCATCGGAGCTGGAATTTGTGCC |
| | | TCTTATCAAACTCAAACCAATTCTCCTAGGAGGGCTAGATCCGTTGCCTCTCAATCTATTATTGCTTATACCATGTCTTT |
| | | AGGAGCTGAAAATTCCGTTGCTTACTCTAACAATTCTATTGCTATTCCTACTAACTTTACTATTTCTGTTACTACTGAAAT |
| | | TCTGCCCGTGTCCATGACTAAAACTTCTGTTGATTGTACTATGTATATTTGTGGAGATTCCACTGAGTGTTCTAACCTG |
| | | TTATTACAATACGGATCTTTTTGTACTCAATTAAATAGAGCTCTGACTGGAATTGCTGTTGAACAGGACAAAAATACTCA |
| | | AGAAGTTTTTGCCCAAGTTAAACAGATTTATAAAACTCCTCCTATTAAAGATTTTGGAGGATTTAATTTCTCTCAAATTTT |
| | | ACCTGACCCTTCTAAGCCTTCCAAAAGGTCTTTCATCGAAGATTTATTATTTAATAAAGTTACCTTAGCCGACGCTGGA |
| | | TTTATTAAGCAATACGGAGATTGCTTAGGCGATATCGCTGCTAGAGACTTAATTTGTGCTCAAAAATTTAACGGATTAA |
| | | CTGTTTTACCTCCCCTGTTAACTGACGAAATGATCGCTCAATATACTTCTGCTCTGTTAGCTGGAACTATTACTTCTGG |
| | | CTGGACTTTTGGAGCTGGAGCTGCTTTACAAATTCCCTTTGCTATGCAGATGGCTTATAGATTTAACGGAATTGGAGTT |
| | | ACTCAAAACGTTTTATACGAAAATCAAAAATTAATTGCTAATCAATTTAATTCTGCTATTGGAAAAATTCAAGATTCCCTG |
| | | TCTTCTACTGCTTCCGCTCTGGGAAAATTACAAGACGTTGTTAATCAAAACGCTCAAGCTTTGAATACCTTAGTTAAAC |
| | | AATTATCTTCCAATTTTGGAGCTATTTCTTCTGTTTTAAACGACATTTTATCTAGATTAGATCCCCCTGAAGCTGAAGTT |
| | | CAGATTGACAGATTGATTACCGGAAGATTACAATCTTTACAAACTTACGTTACTCAACAACTGATTAGAGCTGCTGAAA |
| | | TTAGAGCCTCTGCTAATTTAGCTGCTACTAAGATGTCTGAGTGTGTTTTAGGACAGTCCAAAAGAGTTGATTTTTGTGG |
| | | AAAAGGCTATCATCTGATGTCTTTTCCTCAATCTGCTCCTCACGGAGTTGTGTTTTTACACGTTACTTACGTTCCTGCTC |
| | | AAGAGAAGAATTTTACTACTGCTCCTGCTATTTGTCACGACGGAAAAGCTCATTTTCCTAGAGAAGGAGTTTTTGTTTC |
| | | TAACGGAACTCATTGGTTTGTTACTCAAAGAAACTTTTACGAACCTCAGATTATTACCACTGATAATACCTTTGTTTCTG |
| | | GCAATTGTGACGTGGTTATTGGAATTGTTAATAATACTGTTTACGATCCTTTACAACCCGAACTGGATTCTTTTAAAGAA |
| | | GAATTAGATAAATACTTTAAAAATCATACTTCTCCCGACGTTGATTTAGGAGATATCTCTGGAATTAACGCTTCTGTTGT |
| | | GAATATTCAAAAGGAAATTGATAGATTAAACGAAGTGGCTAAAAATTTAAACGAGTCTTTAATTGATTTACAGGAACTGG |
| | | GAAAATACGAACAATATATTAAGTGGCCTTGGTACATTTGGTTAGGCTTTATTGCTGGACTGATTGCTATTGTGATGGT |
| | | TACTATCATGTTGTGTTGCATGACTTCTTGTTGTTCCTGTTTAAAAGGCTGTTGCTCTTGTGGATCTTGTTGCAAATTTG |
| | | ACGAAGACGATTCTGAACCTGTTCTGAAAGGAGTTAAACTGCATTATACTTAAATGAGACCATA |
| 254 | 2A4581 | ATAGGTCTCACACATTAAAACAGCCTGTGGGTTGATCCCACCCACAGGCCCATTGGGCGCTAGCACTCTGGTATCAC |
| | | GGTACCTTTGTGCGCCTGTTTTATACCCCCTCCCCCAACTGTAACTTAGAAGTAACACACACCGATCAACAGTCAGCG |
| | | TGGCACACCAGCCACGTTTTGATCAAGCACTTCTGTTACCCCGGACTGAGTATCAATAGACTGCTCACGCGGTTGAAG |
| | | GAGAAAGCGTTCGTTATCCGGCCAACTACTTCGAAAAACCTAGTAACACCGTGGAAGTTGCAGAGTGTTTCGCTCAGC |
| | | ACTACCCCAGTGTAGATCAGGTCGATGAGTCACCGCATTCCCCACGGGCGACCGTGGCGGTGGCTGCGTTGGCGGC |
| | | CTGCCCATGGGGAAACCCATGGGACGCTCTAATACAGACATGGTGCGAAGAGTCTATTGAGCTAGTTGGTAGTCCTC |
| | | CGGCCCCTGAATGCGGCTAATCCTAACTGCGGAGCACACACCCTCAAGCCAGAGGGCAGTGTGTCGTAACGGGCAA |
| | | CTCTGCAGCGGAACCGACTACTTTGGGTGTCCGTGTTTCATTTTATTCCTATACTGGCTGCTTATGGTGACAATTGAGA |
| | | GATCGTTACCATATAGCTATTGGATTGGCCATCCGGTGACTAATAGAGCTATTATATATCCCTTTGTTGGGTTTATACC |
| | | ACTTAGCTTGAAAGAGGTTAAAACATTACAATTCATTGTTAAGTTGAATACAGCAACATGTTCGTGTTCTTAGTGCTGTT |
| | | ACCCCTGGTTTCTTCTCAGTGTGTCAATTTAACCACCAGAACCCAACTGCCTCCTGCCTATACTAATTCCTTTACCCGC |
| | | GGAGTTTACTATCCCGATAAGGTTTTTCGCTCCTCTGTTTTACACTCTACTCAGGACTTATTCTTACCCTTCTTCTCCAA |
| | | CGTTACTTGGTTTCACGCCATTCACGTTTCTGGAACCAACGGAACTAAGAGATTCGATAATCCTGTGTTACCTTTCAAC |
| | | GACGGAGTTTATTTTGCTTCCACTGAAAAATCCAATATCATTAGGGGCTGGATCTTTGGAACCACTTTAGATTCCAAGA |
| | | CTCAATCTTTATTAATTGTGAATAACGCCACCAACGTGGTTATCAAAGTGTGTGAATTCCAATTTTGTAACGACCCTTTC |
| | | TTAGGCGTGTATTATCACAAAAATAATAAATCTTGGATGGAATCTGAATTTCGCGTTTACTCTTCCGCTAACAACTGCAC |
| | | TTTTGAGTACGTTTCCCAGCCTTTTCTGATGGATCTGGAAGGCAAACAGGGAAATTTTAAAAATCTGAGAGAATTTGTT |
| | | TTTAAGAACATTGACGGATATTTCAAGATCTATTCTAAACACACTCCCATCAATTTGGTTAGAGATTTACCTCAGGGCTT |
| | | TTCTGCTTTAGAACCTTTAGTTGACCTGCCTATTGGAATCAACATTACTAGATTTCAAACTCTGTTAGCTCTGCATAGAT |
| | | CTTATTTAACTCCTGGAGATTCCTCCTCCGGCTGGACCGCCGGAGCTGCTGCTTATTACGTTGGCTATTTACAACCCA |
| | | GAACTTTTCTGTTAAAATATAACGAAAACGGCACCATCACTGACGCCGTGGATTGTGCTTTAGATCCCTTATCCGAAAC |
| | | TAAGTGTACTCTGAAATCCTTCACTGTTGAAAAAGGAATTTATCAGACCTCTAATTTTAGAGTGCAACCTACCGAATCTA |
| | | TTGTTAGATTTCCTAATATTACCAACTTGTGCCCTTTTGGCGAAGTTTTCAACGCTACTAGATTTGCTTCTGTTTACGCT |
| | | TGGAATAGAAAAAGAATTTCTAACTGTGTGGCTGACTATTCCGTGTTATACAATTCTGCCTCTTTTTCTACTTTTAAGTG |
| | | TTACGGAGTGTCTCCCACTAAACTGAACGATTTGTGCTTTACCAACGTTTACGCTGATTCTTTTGTTATTAGAGGCGAC |
| | | GAGGTTCGCCAGATTGCCCCTGGCCAGACTGGCAAAATTGCTGACTATAACTATAAATTACCTGACGATTTTACCGGC |
| | | TGCGTTATCGCTTGGAACTCTAATAATCTGGATTCTAAAGTTGGAGGAAACTACAATTATTTATATAGATTATTTAGAAA |
| | | ATCTAATTTAAAACCCTTCGAGAGGGATATCTCTACTGAAATCTACCAAGCTGGATCTACTCCTTGCAACGGCGTTGAA |
| | | GGATTTAATTGTTATTTTCCTTTACAATCTTACGGATTCCAACCTACTAACGGAGTTGGATATCAGCCTTATAGAGTGGT |
| | | GGTTTTATCTTTCGAATTATTACACGCTCCCGCTACTGTGTGTGGACCCAAAAAGTCTACTAATTTAGTTAAAAATAAGT |
| | | GTGTTAATTTCAATTTTAACGGACTCACTGGCACTGGAGTGTTAACTGAATCTAATAAAAAGTTCTTACCTTTCCAACAG |
| | | TTCGGACGCGACATTGCCGACACCACTGACGCTGTGAGAGATCCTCAAACCTTAGAGATTCTGGATATTACCCCTTGT |
| | | TCCTTTGGAGGCGTTTCCGTTATCACTCCCGGAACTAACACTTCTAATCAGGTTGCCGTTTTATATCAGGACGTTAATT |
| | | GCACTGAAGTTCCTGTTGCTATTCACGCTGATCAACTGACTCCTACTTGGAGAGTTTATTCCACTGGATCTAACGTTTT |
| | | TCAGACTCGCGCCGGCTGTTTAATTGGAGCTGAACACGTGAACAACTCCTACGAGTGCGATATTCCTATTGGCGCTG |
| | | GCATCTGTGCTTCTTATCAAACTCAAACTAATTCTCCCAGGAGGGCTCGCTCTGTTGCTTCCCAATCTATCATTGCTTA |
| | | TACTATGTCCTTAGGCGCTGAAAATTCTGTTGCTTATTCCAACAATTCTATTGCTATTCCCACCAATTTTACTATTTCTGT |
| | | GACTACTGAAATTTTACCTGTTTCCATGACTAAGACTTCTGTTGATTGTACTATGTATATCTGCGGAGATTCTACTGAGT |
| | | GTTCCAACCTCTTATTACAGTACGGATCTTTTTGCACTCAGTTAAATAGAGCTCTGACTGGAATCGCTGTTGAACAAGA |
| | | CAAAAACACCCAAGAAGTGTTTGCTCAGGTGAAGCAAATTTACAAGACACCTCCTATTAAGGATTTTGGAGGATTCAAT |
| | | TTTTCCCAGATTTTACCTGATCCTTCTAAACCTTCTAAACGCTCCTTTATTGAAGATTTACTGTTCAATAAAGTGACTCT |
| | | GGCTGACGCTGGCTTCATCAAGCAATACGGCGATTGTTTAGGAGACATCGCTGCTAGAGACTTAATTTGTGCTCAAAA |
| | | ATTCAACGGACTGACTGTTCTGCCCCCTTTACTGACCGACGAGATGATCGCCCAATATACTTCTGCCCTGTTAGCTGG |
| | | CACCATTACTTCTGGCTGGACCTTTGGAGCTGGCGCTGCTTTACAAATTCCTTTCGCCATGCAGATGGCTTATCGCTT |
| | | TAACGGAATTGGCGTTACCCAAAACGTGTTATACGAGAATCAAAAATTAATCGCCAATCAATTTAATTCTGCTATTGGC |
| | | AAGATTCAAGATTCTTTATCTTCTACTGCTTCCGCCCTGGGCAAACTGCAGGACGTTGTTAACCAGAACGCTCAAGCC |
| | | CTGAACACTTTAGTGAAGCAACTGTCTTCTAACTTCGGCGCTATTTCTTCCGTTTTAAACGACATTCTGTCTCGCTTAG |
| | | ATCCCCCTGAGGCTGAGGTTCAAATTGATAGACTGATTACTGGAAGATTACAGTCCCTGCAAACTTACGTTACTCAACA |
| | | GTTAATCAGAGCTGCTGAAATCCGCGCTTCCGCTAATTTAGCTGCTACTAAAATGTCTGAGTGTGTGTTAGGCCAGTC |
| | | TAAGAGAGTTGACTTTTGTGGCAAGGGCTATCATTTAATGTCTTTTCCTCAGTCCGCTCCTCACGGCGTTGTGTTCTTA |
| | | CACGTGACTTACGTTCCCGCCCAAGAGAAAAATTTTACCACTGCTCCTGCTATTTGTCACGACGGAAAAGCTCATTTTC |
| | | CTAGAGAAGGAGTGTTTGTTTCTAACGGAACCCACTGGTTTGTGACTCAAAGAAACTTTTACGAACCCCAAATTATTAC |
| | | TACTGACAATACTTTCGTTTCCGGCAACTGTGACGTGGTGATCGGAATTGTTAATAACACCGTTTACGATCCCTTACAA |
| | | CCTGAATTAGATTCTTTTAAAGAAGAATTAGATAAATATTTCAAAAATCATACTTCCCCTGACGTTGATCTGGGAGATAT |
| | | TTCTGGAATTAACGCTTCTGTGGTGAACATTCAAAAAGAGATTGACAGATTAAACGAAGTTGCTAAAAACCTGAACGAG |
| | | TCCCTGATCGACCTGCAGGAACTGGGAAAATACGAGCAGTATATTAAGTGGCCTTGGTATATTTGGTTAGGATTTATT |
| | | GCTGGATTAATTGCTATTGTGATGGTGACCATTATGTTGTGCTGTATGACCTCTTGTTGTTCTTGCCTGAAGGGCTGCT |
| | | GTTCTTGTGGCTCTTGTTGCAAGTTTGACGAAGACGATTCCGAACCCGTTTTAAAGGGAGTGAAACTGCATTATACTTA |
| | | AATGAGACCATA |
| 255 | 2A5581 | ATAGGTCTCACACATTAAAACAGCCTGTGGGTTGATCCCACCCACAGGCCCATTGGGCGCTAGCACTCTGGTATCAC |
| | | GGTACCTTTGTGCGCCTGTTTTATACCCCCTCCCCCAACTGTAACTTAGAAGTAACACACACCGATCAACAGTCAGCG |
| | | TGGCACACCAGCCACGTTTTGATCAAGCACTTCTGTTACCCCGGACTGAGTATCAATAGACTGCTCACGCGGTTGAAG |
| | | GAGAAAGCGTTCGTTATCCGGCCAACTACTTCGAAAAACCTAGTAACACCGTGGAAGTTGCAGAGTGTTTCGCTCAGC |
| | | ACTACCCCAGTGTAGATCAGGTCGATGAGTCACCGCATTCCCCACGGGCGACCGTGGCGGTGGCTGCGTTGGCGGC |
| | | CTGCCCATGGGGAAACCCATGGGACGCTCTAATACAGACATGGTGCGAAGAGTCTATTGAGCTAGTTGGTAGTCCTC |
| | | CGGCCCCTGAATGCGGCTAATCCTAACTGCGGAGCACACACCCTCAAGCCAGAGGGCAGTGTGTCGTAACGGGCAA |
| | | CTCTGCAGCGGAACCGACTACTTTGGGTGTCCGTGTTTCATTTTATTCCTATACTGGCTGCTTATGGTGACAATTGAGA |
| | | GATCGTTACCATATAGCTATTGGATTGGCCATCCGGTGACTAATAGAGCTATTATATATCCCTTTGTTGGGTTTATACC |
| | | ACTTAGCTTGAAAGAGGTTAAAACATTACAATTCATTGTTAAGTTGAATACAGCAACATGTTTGTTTTTCTGGTTTTATTA |
| | | CCCTTAGTGTCCTCCCAGTGCGTGAACTTAACTACCCGCACTCAGCTGCCTCCTGCCTATACCAACTCTTTCACTAGA |
| | | GGCGTTTACTATCCTGACAAAGTTTTTAGATCTTCTGTGCTGCATTCCACTCAAGATTTATTCCTGCCCTTCTTTTCTAA |
| | | CGTTACTTGGTTTCACGCCATCCACGTGTCTGGCACCAACGGAACCAAACGCTTTGACAACCCTGTGTTACCTTTTAA |
| | | CGACGGAGTTTACTTCGCCTCCACTGAAAAGTCCAATATTATCCGGGGCTGGATTTTCGGAACCACCTTAGATTCTAA |
| | | AACCCAGTCTCTGCTGATTGTGAACAACGCCACTAACGTGGTTATTAAAGTGTGCGAGTTTCAATTTTGCAACGACCCT |
| | | TTTCTGGGAGTTTATTACCACAAAAATAACAAATCTTGGATGGAGTCTGAATTTAGAGTGTATTCTTCCGCCAATAATTG |
| | | CACCTTCGAGTACGTTTCCCAACCCTTTCTGATGGACTTAGAAGGAAAGCAAGGCAATTTCAAAAACCTGCGCGAATT |
| | | TGTGTTCAAAAATATTGACGGCTATTTCAAAATTTACTCCAAGCATACCCCTATTAATTTAGTGCGCGATCTGCCTCAG |
| | | GGCTTCTCTGCCTTAGAGCCTTTAGTGGACCTGCCTATTGGCATTAATATTACTCGCTTTCAGACTTTACTGGCCCTGC |
| | | ATAGATCTTATTTAACTCCTGGAGACTCTTCTTCCGGCTGGACCGCTGGAGCCGCTGCCTACTACGTTGGCTATCTGC |
| | | AGCCTAGAACTTTTTTATTAAAGTACAACGAAAACGGAACCATCACTGACGCTGTTGATTGCGCCCTGGATCCTTTATC |
| | | TGAAACAAAGTGCACCCTGAAGTCCTTCACCGTTGAAAAGGGCATTTACCAAACATCCAATTTTAGGGTTCAGCCTACT |
| | | GAATCCATCGTGCGCTTTCCCAACATCACTAACTTGTGTCCCTTTGGCGAGGTGTTTAACGCCACTCGCTTTGCCTCT |
| | | GTGTACGCCTGGAACAGAAAGAGAATTTCCAATTGCGTGGCTGATTATTCTGTGCTGTATAACTCCGCTTCTTTCTCCA |
| | | CCTTTAAGTGTTACGGAGTGTCCCCTACTAAACTGAACGACTTGTGCTTCACCAACGTTTACGCCGATTCTTTTGTGAT |
| | | TCGCGGCGACGAAGTTAGACAAATCGCCCCCGGACAGACCGGAAAGATCGCTGATTACAACTATAAGTTACCTGACG |
| | | ACTTCACCGGCTGCGTGATTGCTTGGAATTCTAATAACTTAGACTCTAAAGTTGGAGGCAACTACAATTACCTGTATAG |
| | | ATTATTCCGCAAATCTAACCTGAAACCTTTTGAGAGAGACATCTCCACCGAAATTTACCAAGCCGGCTCTACCCCCTG |
| | | CAACGGAGTTGAAGGATTTAATTGCTACTTTCCCCTGCAGTCCTACGGATTCCAACCTACTAACGGCGTTGGATACCA |
| | | ACCTTACAGAGTTGTGGTGCTGTCTTTTGAATTATTACACGCCCCTGCCACCGTTTGTGGCCCTAAGAAATCTACCAAT |
| | | CTGGTTAAAAACAAGTGTGTGAATTTTAATTTTAACGGCCTGACCGGCACTGGCGTTTTAACCGAGTCTAACAAGAAGT |
| | | TTCTGCCTTTCCAACAATTTGGCAGAGACATCGCCGACACCACCGACGCTGTGCGCGATCCTCAAACCTTAGAAATTT |
| | | TAGATATTACTCCTTGCTCTTTCGGCGGCGTTTCCGTTATTACTCCTGGAACCAATACCTCTAATCAAGTGGCTGTGCT |
| | | GTACCAGGACGTTAATTGCACTGAAGTGCCTGTGGCTATTCACGCCGATCAACTGACCCCCACTTGGAGAGTTTATTC |
| | | TACTGGATCTAACGTGTTCCAAACTAGAGCTGGCTGTCTGATCGGCGCCGAGCACGTTAATAATTCTTACGAGTGTGA |
| | | TATCCCTATTGGCGCTGGCATTTGCGCCTCTTACCAGACCCAGACCAACTCCCCCCGCAGAGCTAGATCCGTGGCCT |
| | | CTCAGTCCATTATTGCCTATACTATGTCCTTAGGAGCCGAAAATTCTGTGGCTTACTCCAACAATTCTATTGCCATTCC |
| | | CACTAATTTTACTATCTCTGTGACTACCGAAATCCTGCCCGTGTCCATGACTAAGACCTCCGTGGACTGCACCATGTAT |
| | | ATCTGCGGAGACTCTACTGAGTGTTCCAACTTATTACTGCAGTACGGCTCTTTCTGTACTCAGCTGAATAGAGCCCTG |
| | | ACTGGAATTGCTGTGGAGCAAGACAAAAATACCCAGGAGGTGTTCGCTCAGGTGAAACAAATTTATAAGACCCCTCCC |
| | | ATCAAAGATTTTGGAGGCTTCAATTTTTCTCAAATTCTGCCCGACCCCTCCAAGCCTTCCAAAAGATCCTTTATTGAAG |
| | | ATCTGCTGTTCAATAAGGTTACTTTAGCCGACGCCGGCTTCATTAAGCAGTACGGAGATTGTCTGGGCGATATTGCTG |
| | | CCCGCGACCTGATTTGCGCTCAGAAGTTCAACGGATTAACCGTTCTGCCTCCTCTGCTGACTGACGAGATGATTGCTC |
| | | AGTATACTTCTGCCCTGCTGGCTGGAACTATTACCTCTGGCTGGACCTTCGGCGCCGGAGCTGCTCTGCAAATTCCC |
| | | TTCGCCATGCAAATGGCTTACAGATTTAACGGCATCGGAGTTACTCAAAACGTGCTGTACGAAAACCAAAAATTAATTG |
| | | CCAACCAGTTCAATTCCGCTATCGGCAAAATTCAGGATTCTCTGTCTTCTACTGCTTCCGCTTTAGGCAAACTGCAAGA |
| | | CGTTGTTAACCAGAACGCCCAAGCTTTAAACACCCTGGTTAAGCAGCTGTCCTCTAATTTTGGCGCTATCTCTTCTGTT |
| | | CTGAACGACATTTTATCTAGACTGGACCCCCCTGAAGCCGAGGTGCAGATTGATAGATTAATCACCGGAAGACTGCAA |
| | | TCCCTGCAGACCTACGTTACCCAGCAATTAATCAGAGCCGCCGAGATCAGAGCCTCTGCCAACTTAGCTGCCACCAA |
| | | AATGTCCGAGTGTGTGCTGGGACAGTCCAAGCGCGTGGACTTCTGTGGAAAGGGATACCACCTGATGTCTTTTCCTC |
| | | AATCTGCTCCCCACGGAGTTGTGTTTCTGCACGTTACTTACGTGCCTGCCCAAGAGAAAAACTTTACTACCGCCCCCG |
| | | CTATTTGCCACGACGGCAAGGCTCACTTCCCTAGAGAAGGAGTTTTTGTGTCTAACGGAACCCATTGGTTTGTGACTC |
| | | AAAGAAATTTTTACGAGCCTCAAATCATTACCACCGATAACACTTTTGTTTCCGGCAATTGTGACGTGGTGATTGGAAT |
| | | CGTTAACAACACCGTTTACGACCCTTTACAGCCCGAATTAGACTCTTTTAAAGAGGAGTTAGATAAATATTTTAAAAACC |
| | | ATACCTCTCCCGACGTGGACCTGGGAGATATTTCCGGAATTAACGCTTCTGTTGTGAATATCCAGAAAGAGATCGACA |
| | | GATTAAACGAGGTTGCTAAAAATTTAAACGAATCCCTGATTGACCTGCAGGAGCTGGGCAAATACGAACAATATATTAA |
| | | GTGGCCTTGGTATATTTGGTTAGGCTTCATCGCTGGATTAATCGCTATTGTTATGGTGACTATTATGTTGTGTTGTATG |
| | | ACTTCCTGCTGTTCTTGCTTAAAAGGCTGCTGTTCCTGCGGATCCTGTTGTAAATTCGACGAGGACGATTCCGAGCCT |
| | | GTTTTAAAAGGAGTGAAATTACATTATACTTAAATGAGACCATA |
| 256 | 2A6581 | ATAGGTCTCACACATTAAAACAGCCTGTGGGTTGATCCCACCCACAGGCCCATTGGGCGCTAGCACTCTGGTATCAC |
| | | GGTACCTTTGTGCGCCTGTTTTATACCCCCTCCCCCAACTGTAACTTAGAAGTAACACACACCGATCAACAGTCAGCG |
| | | TGGCACACCAGCCACGTTTTGATCAAGCACTTCTGTTACCCCGGACTGAGTATCAATAGACTGCTCACGCGGTTGAAG |
| | | GAGAAAGCGTTCGTTATCCGGCCAACTACTTCGAAAAACCTAGTAACACCGTGGAAGTTGCAGAGTGTTTCGCTCAGC |
| | | ACTACCCCAGTGTAGATCAGGTCGATGAGTCACCGCATTCCCCACGGGCGACCGTGGCGGTGGCTGCGTTGGCGGC |
| | | CTGCCCATGGGGAAACCCATGGGACGCTCTAATACAGACATGGTGCGAAGAGTCTATTGAGCTAGTTGGTAGTCCTC |
| | | CGGCCCCTGAATGCGGCTAATCCTAACTGCGGAGCACACACCCTCAAGCCAGAGGGCAGTGTGTCGTAACGGGCAA |
| | | CTCTGCAGCGGAACCGACTACTTTGGGTGTCCGTGTTTCATTTTATTCCTATACTGGCTGCTTATGGTGACAATTGAGA |
| | | GATCGTTACCATATAGCTATTGGATTGGCCATCCGGTGACTAATAGAGCTATTATATATCCCTTTGTTGGGTTTATACC |
| | | ACTTAGCTTGAAAGAGGTTAAAACATTACAATTCATTGTTAAGTTGAATACAGCAACATGTTCGTGTTCCTGGTGCTGC |
| | | TGCCCTTAGTGTCCTCCCAGTGTGTGAACCTGACCACCAGAACCCAGTTACCTCCCGCTTACACCAACTCCTTTACCC |
| | | GCGGAGTGTACTACCCTGATAAGGTTTTTAGATCCTCCGTTCTGCATTCTACCCAAGACCTGTTTTTACCCTTCTTTTC |
| | | CAACGTGACCTGGTTCCACGCTATCCACGTGTCCGGCACTAACGGCACTAAGCGCTTCGACAATCCTGTGCTCCCTT |
| | | TTAACGACGGCGTGTACTTCGCTTCTACCGAAAAATCCAACATCATCCGCGGCTGGATCTTCGGAACTACCCTGGATT |
| | | CTAAAACCCAGTCTTTATTAATCGTGAATAACGCCACCAACGTGGTTATTAAGGTCTGTGAGTTCCAATTTTGCAACGA |
| | | CCCTTTCTTAGGCGTTTATTACCACAAAAACAATAAGTCTTGGATGGAGTCCGAATTCCGCGTGTACTCCTCTGCCAAC |
| | | AATTGCACCTTTGAGTACGTGTCCCAGCCCTTCCTGATGGACCTGGAAGGCAAGCAGGGCAATTTTAAAAATCTGCGC |
| | | GAATTCGTTTTCAAGAACATCGACGGCTACTTTAAGATTTATTCCAAACATACCCCCATCAACCTGGTGCGCGATTTAC |
| | | CCCAGGGCTTCTCCGCCTTAGAGCCCTTAGTGGATCTGCCTATCGGCATCAATATCACCCGCTTTCAGACTCTGCTGG |
| | | CCCTGCACAGATCCTACCTGACTCCCGGAGACTCTTCTTCTGGCTGGACCGCCGGAGCTGCCGCCTACTACGTGGG |
| | | CTACCTGCAACCCCGCACCTTTCTGCTGAAGTACAACGAAAACGGCACCATCACAGACGCCGTTGACTGCGCTCTGG |
| | | ACCCCCTGTCTGAGACTAAGTGCACCCTGAAGTCCTTCACTGTGGAGAAAGGAATCTACCAGACCTCCAACTTCCGC |
| | | GTGCAGCCTACCGAATCCATTGTTCGCTTCCCTAACATTACTAATCTGTGCCCCTTCGGAGAAGTTTTCAACGCCACTA |
| | | GATTCGCTTCCGTGTACGCCTGGAATCGCAAGCGCATCTCCAATTGCGTTGCTGACTACTCTGTGTTATACAATTCCG |
| | | CCTCTTTTTCCACTTTCAAGTGCTACGGCGTGTCTCCTACCAAGCTGAACGACCTGTGTTTCACTAACGTGTACGCTG |
| | | ATTCCTTTGTTATCCGCGGCGACGAGGTGAGACAGATCGCCCCTGGCCAGACCGGAAAAATCGCCGACTACAACTAC |
| | | AAGTTACCCGACGATTTTACCGGCTGTGTTATTGCTTGGAACTCTAATAATCTGGACTCTAAAGTGGGGGGCAATTACA |
| | | ACTACTTATATCGCCTGTTCAGAAAATCCAACCTGAAACCCTTCGAACGGGACATTTCCACCGAAATTTATCAGGCCG |
| | | GCTCCACCCCCTGCAACGGAGTGGAGGGCTTCAATTGCTACTTCCCCCTGCAATCCTACGGCTTTCAGCCCACCAAC |
| | | GGCGTGGGATACCAGCCCTACCGCGTTGTTGTGTTATCCTTTGAACTGCTGCACGCTCCTGCTACTGTGTGCGGACC |
| | | TAAAAAATCCACCAATCTGGTGAAGAACAAGTGTGTGAATTTTAATTTTAACGGCCTGACCGGCACCGGAGTGCTGAC |
| | | CGAGTCCAACAAAAAGTTTCTGCCCTTTCAGCAGTTCGGCCGGGACATTGCCGACACTACCGACGCTGTGAGGGATC |
| | | CTCAGACCCTGGAGATTCTGGACATTACCCCTTGCTCCTTCGGAGGAGTTTCCGTGATCACCCCTGGCACCAATACTT |
| | | CTAACCAAGTGGCTGTTTTATACCAGGACGTGAATTGTACTGAGGTGCCTGTGGCCATCCACGCCGATCAATTAACTC |
| | | CCACCTGGAGAGTGTATTCCACCGGCTCTAACGTTTTCCAAACCCGCGCTGGCTGCTTAATCGGCGCTGAGCACGTG |
| | | AACAACTCCTACGAGTGTGATATTCCTATCGGAGCCGGCATCTGTGCTTCCTACCAGACTCAGACCAACTCCCCCAGA |
| | | CGCGCTAGGTCCGTGGCTTCCCAGTCTATTATCGCTTATACTATGTCCCTGGGAGCCGAGAATTCTGTTGCCTATTCC |
| | | AATAACTCCATCGCCATTCCTACCAACTTTACCATCTCTGTGACCACCGAGATTCTGCCCGTGTCCATGACCAAAACTT |
| | | CTGTGGATTGTACCATGTACATCTGTGGAGATTCCACCGAGTGCTCTAACCTGCTGCTGCAGTACGGCTCCTTTTGCA |
| | | CCCAATTAAACCGCGCTCTGACTGGCATCGCCGTGGAGCAAGATAAAAACACCCAAGAGGTGTTCGCTCAGGTGAAG |
| | | CAGATCTACAAGACACCTCCCATTAAAGATTTCGGAGGCTTTAATTTCTCTCAGATCCTGCCCGACCCTTCTAAGCCCT |
| | | CTAAACGCTCCTTCATCGAGGATCTGCTGTTCAACAAGGTTACCCTGGCCGACGCTGGCTTTATCAAACAGTACGGCG |
| | | ACTGCTTAGGCGACATCGCCGCCCGCGATTTAATTTGCGCTCAGAAGTTCAACGGACTGACCGTGCTGCCTCCTCTG |
| | | CTGACCGACGAAATGATCGCCCAATACACCTCCGCCCTGCTGGCTGGCACTATCACCTCCGGCTGGACCTTTGGCGC |
| | | TGGAGCCGCTCTGCAAATCCCTTTCGCTATGCAGATGGCTTACCGCTTTAACGGAATCGGCGTGACCCAGAACGTGT |
| | | TATACGAAAATCAGAAATTAATTGCCAATCAATTTAACTCTGCCATCGGCAAGATTCAGGATTCTCTGTCTTCTACTGCC |
| | | TCTGCCTTAGGAAAACTGCAGGACGTGGTGAATCAGAACGCCCAGGCCCTGAATACCCTGGTTAAGCAACTGTCTTC |
| | | CAATTTCGGCGCTATCTCCTCTGTGTTAAACGACATTCTGTCCCGCCTGGATCCTCCTGAGGCCGAAGTGCAAATTGA |
| | | CCGCTTAATCACTGGCAGACTGCAATCCCTGCAAACCTACGTTACTCAACAGCTGATCAGAGCCGCCGAAATCCGCG |
| | | CTTCTGCCAACTTAGCTGCTACCAAGATGTCCGAGTGCGTGCTGGGACAGTCCAAAAGAGTTGACTTTTGCGGAAAG |
| | | GGCTACCACCTGATGTCCTTTCCCCAGTCCGCCCCCCACGGAGTTGTTTTCCTGCACGTTACTTACGTTCCTGCTCAA |
| | | GAGAAGAACTTCACCACCGCTCCCGCTATTTGCCACGACGGCAAGGCTCATTTCCCCAGAGAGGGAGTGTTTGTGTC |
| | | CAACGGCACTCACTGGTTTGTTACTCAGCGCAACTTCTACGAACCTCAGATCATCACCACTGACAATACCTTTGTGTC |
| | | CGGAAACTGTGACGTGGTTATCGGCATTGTGAACAATACTGTGTACGACCCTTTACAACCTGAGCTGGATTCTTTTAAA |
| | | GAAGAACTGGACAAGTACTTCAAGAACCACACCTCCCCCGACGTGGATCTGGGCGACATCTCCGGCATCAACGCCTC |
| | | TGTGGTGAATATTCAGAAGGAGATTGATCGCCTGAACGAGGTTGCCAAGAATCTGAACGAGTCTCTGATCGACCTGCA |
| | | GGAGCTGGGCAAGTACGAACAGTACATCAAGTGGCCCTGGTACATTTGGCTGGGCTTTATTGCTGGACTGATCGCTA |
| | | TCGTTATGGTTACCATCATGCTGTGCTGTATGACCTCTTGCTGCTCCTGTCTGAAAGGCTGTTGCTCCTGCGGCTCCT |
| | | GTTGTAAGTTTGACGAGGACGACTCCGAGCCTGTTCTGAAAGGAGTTAAACTGCACTACACCTAAATGAGACCATA |
| 257 | 2A7581 | ATAGGTCTCACACATTAAAACAGCCTGTGGGTTGATCCCACCCACAGGCCCATTGGGCGCTAGCACTCTGGTATCAC |
| | | GGTACCTTTGTGCGCCTGTTTTATACCCCCTCCCCCAACTGTAACTTAGAAGTAACACACACCGATCAACAGTCAGCG |
| | | TGGCACACCAGCCACGTTTTGATCAAGCACTTCTGTTACCCCGGACTGAGTATCAATAGACTGCTCACGCGGTTGAAG |
| | | GAGAAAGCGTTCGTTATCCGGCCAACTACTTCGAAAAACCTAGTAACACCGTGGAAGTTGCAGAGTGTTTCGCTCAGC |
| | | ACTACCCCAGTGTAGATCAGGTCGATGAGTCACCGCATTCCCCACGGGCGACCGTGGCGGTGGCTGCGTTGGCGGC |
| | | CTGCCCATGGGGAAACCCATGGGACGCTCTAATACAGACATGGTGCGAAGAGTCTATTGAGCTAGTTGGTAGTCCTC |
| | | CGGCCCCTGAATGCGGCTAATCCTAACTGCGGAGCACACACCCTCAAGCCAGAGGGCAGTGTGTCGTAACGGGCAA |
| | | CTCTGCAGCGGAACCGACTACTTTGGGTGTCCGTGTTTCATTTTATTCCTATACTGGCTGCTTATGGTGACAATTGAGA |
| | | GATCGTTACCATATAGCTATTGGATTGGCCATCCGGTGACTAATAGAGCTATTATATATCCCTTTGTTGGGTTTATACC |
| | | ACTTAGCTTGAAAGAGGTTAAAACATTACAATTCATTGTTAAGTTGAATACAGCAACATGTTTGTGTTTCTGGTGCTGCT |
| | | GCCTCTGGTGTCCTCTCAGTGTGTTAACCTGACCACCAGAACCCAACTGCCCCCCGCCTACACTAACTCTTTCACTCG |
| | | CGGCGTGTACTACCCCGATAAAGTTTTCAGATCCTCCGTGCTCCATTCTACCCAAGACCTGTTCTTACCCTTTTTCTCC |
| | | AACGTGACCTGGTTCCACGCCATCCACGTGTCCGGCACTAACGGAACCAAGCGCTTCGATAATCCCGTGCTGCCTTT |
| | | CAACGACGGAGTTTACTTTGCTTCCACCGAGAAGTCCAATATTATCCGGGGCTGGATCTTCGGCACCACACTGGATTC |
| | | CAAAACCCAGTCCTTACTGATTGTGAACAACGCCACTAACGTGGTGATCAAGGTGTGTGAGTTCCAGTTCTGTAACGA |
| | | CCCCTTTCTGGGCGTGTATTATCATAAAAACAACAAGTCCTGGATGGAGTCCGAGTTTAGAGTGTATTCTTCCGCTAAC |
| | | AACTGCACCTTCGAATACGTGTCCCAGCCTTTCCTGATGGATCTGGAAGGAAAGCAGGGCAACTTCAAGAACTTACGC |
| | | GAGTTCGTGTTCAAGAACATCGACGGCTACTTCAAAATTTACTCCAAGCACACCCCTATCAACTTAGTGAGAGATCTG |
| | | CCCCAGGGCTTCTCCGCTCTGGAACCTCTGGTGGACTTACCCATTGGCATCAACATCACTCGCTTCCAAACACTGCTC |
| | | GCTCTGCACCGCTCTTATCTGACCCCCGGCGACTCCTCTTCTGGCTGGACCGCCGGCGCCGCTGCCTATTACGTGG |
| | | GATACCTGCAACCCCGCACCTTTCTGCTGAAATACAACGAAAACGGCACCATCACAGACGCTGTGGACTGCGCTCTG |
| | | GACCCCCTGTCCGAGACTAAGTGCACCCTGAAGTCCTTCACCGTTGAAAAGGGCATTTATCAGACTTCCAACTTCCGC |
| | | GTGCAGCCTACTGAGTCCATTGTGCGCTTCCCTAACATCACTAACTTGTGTCCTTTCGGCGAAGTGTTCAACGCTACT |
| | | CGCTTCGCCTCCGTGTACGCCTGGAACCGCAAGAGAATCTCCAATTGCGTGGCCGACTACTCTGTTCTCTACAACTCT |
| | | GCCTCCTTCTCCACCTTTAAGTGCTACGGCGTGTCCCCCACCAAACTGAACGACCTGTGCTTCACCAACGTGTACGC |
| | | CGACTCCTTTGTGATCAGAGGCGACGAGGTGCGCCAGATCGCCCCCGGCCAGACTGGCAAGATCGCCGATTACAAT |
| | | TACAAGCTGCCTGACGACTTTACCGGCTGCGTGATTGCCTGGAACTCCAACAACCTGGACTCCAAAGTGGGCGGCAA |
| | | CTACAACTACCTGTACCGCCTGTTTAGAAAATCCAACCTGAAGCCCTTCGAGCGGGACATTTCTACTGAGATCTACCA |
| | | GGCCGGCTCCACCCCCTGTAACGGCGTGGAAGGCTTCAACTGCTACTTTCCCCTGCAGTCCTACGGCTTCCAGCCTA |
| | | CCAACGGCGTGGGCTACCAGCCCTACCGCGTGGTGGTGTTATCCTTCGAGCTGCTGCACGCCCCTGCCACCGTGTG |
| | | CGGACCCAAAAAGTCTACTAACCTGGTTAAGAATAAGTGTGTTAATTTCAATTTTAACGGCTTAACCGGAACCGGAGTG |
| | | CTGACCGAGTCCAATAAAAAGTTCCTGCCCTTTCAGCAGTTTGGCCGCGACATCGCCGATACTACCGACGCCGTGCG |
| | | CGATCCCCAGACCCTGGAGATCCTGGACATTACCCCCTGCTCTTTCGGCGGAGTGTCCGTGATCACCCCCGGCACCA |
| | | ATACCTCCAATCAGGTGGCCGTTCTGTACCAGGACGTGAATTGCACCGAAGTGCCCGTGGCCATTCACGCCGACCAG |
| | | TTAACTCCTACTTGGAGAGTGTACTCCACCGGCTCTAACGTGTTTCAGACCCGCGCCGGCTGTCTGATCGGCGCCGA |
| | | GCACGTGAACAACTCCTACGAGTGCGACATTCCCATTGGCGCCGGCATCTGCGCCTCCTACCAAACCCAGACTAACT |
| | | CTCCCAGACGCGCCCGCTCTGTGGCCTCTCAGTCCATCATCGCTTACACTATGTCCCTGGGCGCCGAGAACTCCGTG |
| | | GCCTATTCCAACAACTCCATCGCCATCCCCACTAACTTCACCATCTCTGTGACCACCGAAATCCTCCCCGTGTCTATG |
| | | ACTAAGACTTCCGTGGACTGTACCATGTACATTTGCGGCGACTCCACCGAGTGTTCCAATCTGCTGCTGCAGTACGG |
| | | CTCCTTTTGCACCCAGCTGAACCGCGCCCTGACCGGCATCGCTGTTGAGCAGGACAAGAATACCCAGGAGGTGTTTG |
| | | CTCAGGTGAAGCAAATCTATAAGACCCCTCCCATCAAGGACTTTGGCGGCTTCAACTTTTCCCAGATCCTGCCCGACC |
| | | CTTCCAAGCCCTCCAAACGCTCTTTCATCGAAGATCTGCTGTTCAATAAAGTGACCCTGGCCGACGCCGGCTTCATCA |
| | | AGCAGTACGGAGACTGTCTGGGAGACATTGCTGCTCGCGACCTGATTTGCGCCCAGAAGTTCAACGGCCTGACCGT |
| | | GCTGCCCCCTCTGCTGACCGACGAGATGATCGCTCAGTACACCTCCGCCCTGCTGGCTGGCACTATCACCTCCGGCT |
| | | GGACTTTCGGCGCTGGCGCCGCCCTGCAGATTCCCTTCGCCATGCAGATGGCTTATCGCTTCAACGGAATCGGAGTG |
| | | ACACAGAACGTGCTGTACGAGAATCAGAAACTGATCGCTAACCAGTTTAATTCCGCCATTGGCAAGATCCAGGACTCC |
| | | CTGTCCTCTACTGCTTCCGCCCTGGGCAAGCTGCAGGACGTGGTGAACCAGAACGCCCAGGCTCTGAACACCCTGG |
| | | TGAAGCAACTGTCTTCTAACTTTGGAGCCATTTCTTCCGTGCTGAACGACATCCTGTCCAGACTGGATCCTCCTGAGG |
| | | CCGAGGTGCAGATCGACCGCCTGATCACTGGCCGCCTGCAGTCCCTGCAGACTTACGTTACCCAGCAATTAATCCGC |
| | | GCTGCCGAGATCCGCGCCTCCGCCAACCTGGCCGCCACCAAGATGTCCGAGTGCGTTCTGGGCCAGTCCAAACGCG |
| | | TTGACTTCTGCGGCAAGGGCTACCACCTGATGTCCTTCCCCCAGTCCGCTCCCCACGGAGTGGTGTTCCTGCACGTG |
| | | ACCTACGTGCCTGCCCAGGAGAAGAACTTCACCACCGCCCCCGCCATTTGCCACGACGGAAAGGCCCACTTCCCCC |
| | | GCGAGGGAGTGTTCGTTTCTAACGGCACTCACTGGTTCGTGACCCAGAGAAACTTCTACGAGCCCCAGATTATTACCA |
| | | CCGACAATACCTTCGTTTCTGGCAACTGCGACGTGGTTATCGGCATCGTGAACAACACCGTGTACGACCCCCTGCAA |
| | | CCCGAGCTGGATTCTTTCAAGGAGGAGCTGGATAAGTACTTTAAAAACCATACCTCCCCCGACGTGGACCTGGGCGA |
| | | CATCTCTGGCATCAACGCCTCCGTGGTGAATATCCAGAAGGAGATCGATCGCCTGAACGAGGTGGCCAAGAACTTAA |
| | | ACGAATCTCTGATCGACCTGCAGGAGCTGGGCAAGTACGAGCAGTATATTAAGTGGCCCTGGTATATCTGGTTAGGC |
| | | TTTATCGCCGGCCTGATCGCCATTGTTATGGTGACCATCATGCTGTGCTGCATGACCTCCTGCTGTTCCTGTTTAAAA |
| | | GGCTGCTGCTCCTGCGGATCCTGCTGTAAATTCGACGAAGACGACTCCGAGCCCGTGCTGAAAGGAGTTAAGTTACA |
| | | CTATACCTAAATGAGACCATA |
| 258 | 2A8581 | ATAGGTCTCACACATTAAAACAGCCTGTGGGTTGATCCCACCCACAGGCCCATTGGGCGCTAGCACTCTGGTATCAC |
| | | GGTACCTTTGTGCGCCTGTTTTATACCCCCTCCCCCAACTGTAACTTAGAAGTAACACACACCGATCAACAGTCAGCG |
| | | TGGCACACCAGCCACGTTTTGATCAAGCACTTCTGTTACCCCGGACTGAGTATCAATAGACTGCTCACGCGGTTGAAG |
| | | GAGAAAGCGTTCGTTATCCGGCCAACTACTTCGAAAAACCTAGTAACACCGTGGAAGTTGCAGAGTGTTTCGCTCAGC |
| | | ACTACCCCAGTGTAGATCAGGTCGATGAGTCACCGCATTCCCCACGGGCGACCGTGGCGGTGGCTGCGTTGGCGGC |
| | | CTGCCCATGGGGAAACCCATGGGACGCTCTAATACAGACATGGTGCGAAGAGTCTATTGAGCTAGTTGGTAGTCCTC |
| | | CGGCCCCTGAATGCGGCTAATCCTAACTGCGGAGCACACACCCTCAAGCCAGAGGGCAGTGTGTCGTAACGGGCAA |
| | | CTCTGCAGCGGAACCGACTACTTTGGGTGTCCGTGTTTCATTTTATTCCTATACTGGCTGCTTATGGTGACAATTGAGA |
| | | GATCGTTACCATATAGCTATTGGATTGGCCATCCGGTGACTAATAGAGCTATTATATATCCCTTTGTTGGGTTTATACC |
| | | ACTTAGCTTGAAAGAGGTTAAAACATTACAATTCATTGTTAAGTTGAATACAGCAACATGTTCGTGTTCCTGGTGCTGC |
| | | TGCCCCTGGTGTCCTCCCAGTGCGTCAACCTGACCACCCGGACCCAACTGCCCCCTGCCTATACCAATTCCTTCACC |
| | | CGGGGCGTCTACTACCCCGACAAGGTGTTCCGGTCCTCTGTGCTCCACTCCACCCAGGACCTCTTCCTCCCCTTTTT |
| | | CTCCAACGTGACCTGGTTCCACGCCATCCACGTGTCTGGCACAAACGGAACCAAGCGCTTCGACAACCCTGTGCTCC |
| | | CCTTCAACGACGGCGTGTACTTCGCCTCCACCGAGAAGTCCAACATCATCCGCGGCTGGATCTTCGGCACCACCCTC |
| | | GACTCTAAGACCCAGTCCCTGCTGATTGTGAATAACGCCACCAACGTGGTGATCAAGGTGTGCGAATTTCAGTTCTGC |
| | | AACGACCCCTTCCTGGGCGTTTACTACCATAAGAACAACAAGTCCTGGATGGAGTCCGAGTTCCGGGTGTATTCCTCT |
| | | GCCAATAACTGCACCTTTGAGTACGTGTCCCAGCCCTTCCTGATGGACCTGGAGGGCAAGCAGGGCAATTTTAAAAA |
| | | CCTGCGCGAGTTCGTGTTCAAGAACATCGACGGCTACTTCAAGATCTATTCCAAGCACACCCCTATCAACCTGGTGCG |
| | | CGACCTGCCCCAGGGCTTTTCCGCTCTGGAGCCCCTGGTTGACCTGCCCATCGGCATCAACATCACCCGCTTCCAGA |
| | | CTTTACTGGCCCTGCACCGCTCCTACCTGACCCCCGGCGATTCCTCTTCTGGCTGGACAGCCGGAGCCGCTGCCTAC |
| | | TACGTGGGATACCTGCAGCCCCGCACCTTCCTGCTGAAGTACAACGAGAACGGAACCATTACAGACGCTGTCGACTG |
| | | TGCTCTGGATCCCCTGTCCGAAACAAAGTGCACCCTGAAGTCCTTCACCGTGGAGAAGGGCATCTACCAGACCTCCA |
| | | ACTTCCGCGTGCAGCCCACCGAGTCCATCGTGAGATTCCCCAACATCACTAACCTGTGCCCCTTCGGCGAGGTGTTC |
| | | AACGCCACCCGCTTCGCCTCCGTGTACGCCTGGAACCGCAAGCGCATCTCCAACTGCGTTGCCGACTACTCTGTGCT |
| | | CTACAACTCCGCCTCCTTCTCCACCTTCAAGTGCTACGGCGTGTCCCCCACCAAGCTGAACGACCTGTGCTTCACCAA |
| | | CGTGTACGCCGACTCCTTCGTGATCCGCGGCGACGAGGTGCGCCAGATCGCCCCCGGCCAGACCGGAAAGATCGC |
| | | CGATTACAATTACAAGCTGCCCGACGACTTCACCGGCTGCGTGATCGCCTGGAACTCCAACAATCTGGACTCCAAGG |
| | | TTGGCGGCAACTACAACTACCTGTACCGCCTGTTCCGCAAGTCCAACCTGAAGCCCTTCGAGCGGGACATTTCCACA |
| | | GAGATTTACCAGGCTGGCTCCACCCCCTGTAACGGAGTGGAGGGCTTCAACTGCTACTTCCCCCTGCAGTCCTACGG |
| | | CTTCCAGCCCACCAACGGCGTGGGCTACCAGCCCTACCGCGTGGTGGTGCTGTCCTTCGAGCTGCTGCACGCCCCT |
| | | GCCACCGTGTGCGGCCCCAAGAAGTCCACCAACCTGGTGAAGAACAAGTGCGTGAACTTCAACTTCAACGGACTGAC |
| | | AGGCACCGGCGTGCTGACCGAGTCCAACAAGAAGTTCCTGCCCTTCCAGCAGTTCGGCCGGGACATTGCCGACACC |
| | | ACCGACGCCGTGCGCGACCCCCAGACCCTGGAGATCCTGGACATCACCCCCTGCTCCTTCGGCGGAGTGTCCGTGA |
| | | TCACCCCCGGCACCAACACCTCCAACCAGGTTGCCGTGCTGTACCAGGACGTTAACTGCACCGAGGTGCCCGTGGC |
| | | CATCCACGCCGACCAGCTGACCCCCACCTGGCGCGTGTACTCCACCGGCTCCAACGTGTTCCAGACCCGCGCCGGC |
| | | TGCCTGATCGGCGCCGAGCACGTGAACAACTCCTACGAGTGCGACATCCCCATTGGCGCCGGCATCTGCGCCTCCT |
| | | ACCAGACCCAGACCAACTCCCCTCGCCGCGCCCGGTCCGTCGCCTCCCAGTCCATCATCGCCTACACCATGTCCCT |
| | | GGGCGCCGAGAACTCCGTGGCCTACTCCAACAACTCCATTGCCATCCCCACCAACTTCACCATCTCCGTGACCACCG |
| | | AAATCCTCCCCGTGTCCATGACCAAGACCTCCGTGGACTGCACCATGTACATCTGCGGCGACTCCACCGAGTGCTCC |
| | | AACCTGCTGCTGCAGTACGGCTCCTTCTGCACCCAGCTGAATCGCGCCCTGACCGGCATCGCCGTGGAGCAGGACA |
| | | AGAACACCCAAGAGGTGTTCGCCCAGGTGAAGCAGATCTACAAGACACCTCCCATCAAGGACTTCGGCGGCTTCAAC |
| | | TTCTCCCAGATCCTGCCCGACCCCTCCAAGCCCTCCAAGCGCTCCTTCATCGAGGACCTGCTGTTCAACAAGGTGAC |
| | | CCTGGCCGACGCCGGCTTCATCAAGCAGTACGGCGACTGTCTGGGAGACATTGCCGCCCGCGACCTGATCTGCGCC |
| | | CAGAAGTTCAACGGCTTAACCGTGCTGCCCCCTCTGCTGACCGACGAGATGATCGCCCAGTACACATCCGCTCTGCT |
| | | GGCCGGCACCATCACCTCCGGCTGGACCTTTGGCGCTGGCGCTGCCCTGCAGATCCCCTTCGCTATGCAGATGGCC |
| | | TACCGCTTCAACGGCATCGGCGTGACCCAGAACGTGCTGTACGAGAACCAGAAGCTGATCGCCAACCAGTTCAACTC |
| | | CGCCATCGGCAAGATCCAGGACTCCCTGTCCTCCACCGCCTCCGCCCTGGGCAAGCTGCAGGACGTGGTGAACCAG |
| | | AACGCCCAGGCCCTGAACACCCTGGTGAAGCAGCTGTCCTCTAACTTCGGCGCCATCTCCTCCGTGCTGAACGACAT |
| | | TCTGTCCCGCCTGGATCCTCCTGAGGCCGAGGTGCAAATCGATCGCCTCATCACCGGCCGCCTGCAGTCCCTGCAG |
| | | ACCTACGTGACACAGCAGCTGATCCGCGCCGCCGAGATCCGCGCCTCCGCCAACCTGGCCGCCACCAAGATGTCTG |
| | | AGTGCGTGCTGGGCCAGTCCAAACGCGTTGACTTCTGCGGCAAGGGCTACCACCTGATGTCCTTCCCCCAGTCCGC |
| | | CCCCCACGGCGTGGTGTTCCTGCACGTGACCTACGTGCCCGCCCAGGAGAAGAACTTCACTACCGCCCCCGCCATC |
| | | TGCCACGACGGCAAGGCCCACTTCCCCCGCGAGGGCGTGTTCGTGTCCAACGGCACCCACTGGTTCGTGACCCAGC |
| | | GCAACTTCTACGAGCCCCAGATCATCACCACCGACAACACCTTCGTGTCCGGCAACTGCGACGTGGTGATTGGCATC |
| | | GTGAACAACACCGTGTACGACCCCCTGCAGCCCGAGCTGGACTCCTTCAAGGAGGAGCTGGACAAGTACTTTAAGAA |
| | | CCACACCTCCCCCGACGTGGACCTGGGCGACATCTCCGGCATCAACGCCTCCGTGGTGAACATCCAGAAGGAGATC |
| | | GACCGCCTGAACGAGGTGGCCAAGAACCTGAACGAGTCCCTGATCGACCTGCAGGAGCTGGGCAAGTACGAACAGT |
| | | ACATCAAGTGGCCCTGGTACATCTGGCTGGGCTTCATCGCCGGCCTGATCGCCATCGTGATGGTGACCATCATGCTG |
| | | TGCTGCATGACCTCTTGCTGTTCCTGTCTGAAGGGCTGCTGCTCCTGCGGCTCCTGCTGCAAGTTCGACGAGGACGA |
| | | CTCCGAGCCCGTGCTGAAGGGCGTGAAGCTGCACTACACCTAAATGAGACCATA |
| 259 | 2A9581 | ATAGGTCTCACACATTAAAACAGCCTGTGGGTTGATCCCACCCACAGGCCCATTGGGCGCTAGCACTCTGGTATCAC |
| | | GGTACCTTTGTGCGCCTGTTTTATACCCCCTCCCCCAACTGTAACTTAGAAGTAACACACACCGATCAACAGTCAGCG |
| | | TGGCACACCAGCCACGTTTTGATCAAGCACTTCTGTTACCCCGGACTGAGTATCAATAGACTGCTCACGCGGTTGAAG |
| | | GAGAAAGCGTTCGTTATCCGGCCAACTACTTCGAAAAACCTAGTAACACCGTGGAAGTTGCAGAGTGTTTCGCTCAGC |
| | | ACTACCCCAGTGTAGATCAGGTCGATGAGTCACCGCATTCCCCACGGGCGACCGTGGCGGTGGCTGCGTTGGCGGC |
| | | CTGCCCATGGGGAAACCCATGGGACGCTCTAATACAGACATGGTGCGAAGAGTCTATTGAGCTAGTTGGTAGTCCTC |
| | | CGGCCCCTGAATGCGGCTAATCCTAACTGCGGAGCACACACCCTCAAGCCAGAGGGCAGTGTGTCGTAACGGGCAA |
| | | CTCTGCAGCGGAACCGACTACTTTGGGTGTCCGTGTTTCATTTTATTCCTATACTGGCTGCTTATGGTGACAATTGAGA |
| | | GATCGTTACCATATAGCTATTGGATTGGCCATCCGGTGACTAATAGAGCTATTATATATCCCTTTGTTGGGTTTATACC |
| | | ACTTAGCTTGAAAGAGGTTAAAACATTACAATTCATTGTTAAGTTGAATACAGCAACATGTTCGTGTTCCTGGTGCTGC |
| | | TCCCCCTGGTGAGCTCCCAGTGCGTCAACCTGACCACACGGACCCAGCTGCCCCCCGCCTATACCAACAGCTTCACC |
| | | CGCGGCGTCTACTACCCCGACAAGGTGTTCCGGAGCAGCGTGCTGCACAGCACCCAGGACCTCTTCCTCCCCTTTTT |
| | | TAGCAACGTGACCTGGTTCCACGCCATCCACGTGAGTGGCACCAACGGCACCAAGCGGTTCGACAACCCTGTGCTC |
| | | CCCTTCAACGACGGCGTGTACTTCGCTAGCACAGAAAAAAGTAACATCATTCGGGGCTGGATCTTCGGCACCACACT |
| | | GGATAGCAAAACCCAGAGCCTGCTGATTGTGAATAACGCCACCAACGTGGTGATCAAGGTGTGCGAGTTCCAGTTCT |
| | | GCAACGACCCCTTCCTGGGCGTGTACTACCACAAGAACAACAAGAGCTGGATGGAGAGCGAGTTCCGCGTGTATAGC |
| | | TCTGCCAACAACTGCACCTTCGAGTACGTGAGCCAGCCCTTCCTGATGGACCTGGAGGGCAAGCAGGGCAATTTCAA |
| | | AAACCTGCGGGAGTTCGTGTTCAAGAACATCGACGGCTACTTCAAGATCTACAGCAAGCACACCCCCATCAACCTGG |
| | | TGCGGGACCTGCCCCAGGGCTTTAGCGCTCTGGAGCCCCTGGTGGACCTGCCCATCGGCATCAACATCACCCGCTT |
| | | CCAAACCCTGTTGGCCCTGCACCGGAGCTACCTGACCCCTGGCGATAGCAGTAGTGGCTGGACAGCCGGAGCCGCC |
| | | GCCTACTACGTGGGATACCTGCAGCCCCGGACCTTCCTGCTGAAGTACAACGAGAACGGCACAATCACAGACGCCGT |
| | | GGACTGCGCCCTGGACCCCCTGAGCGAAACAAAGTGCACCCTCAAAAGTTTCACCGTGGAGAAGGGCATCTACCAG |
| | | ACCAGCAACTTCCGGGTGCAGCCCACCGAGAGCATCGTGCGGTTCCCCAACATCACCAACCTGTGCCCCTTCGGCG |
| | | AGGTGTTCAACGCCACCCGGTTCGCCAGCGTGTACGCCTGGAACCGGAAGCGGATCAGCAACTGCGTGGCCGACTA |
| | | CAGCGTGCTGTATAACAGTGCCAGCTTCAGCACCTTCAAGTGCTACGGCGTGAGCCCCACCAAGCTGAACGACCTGT |
| | | GCTTCACCAACGTGTACGCCGACAGCTTCGTGATCCGGGGCGACGAGGTGCGGCAGATCGCCCCCGGCCAGACCG |
| | | GCAAGATCGCCGATTACAATTACAAGCTGCCCGACGATTTCACCGGCTGCGTGATCGCCTGGAACAGCAACAACCTG |
| | | GACAGCAAGGTGGGCGGCAACTACAACTACCTGTACCGGCTGTTCCGCAAAAGTAACCTGAAGCCCTTCGAGCGGG |
| | | ACATCAGCACCGAGATCTACCAGGCCGGCAGTACCCCTTGCAACGGCGTCGAGGGCTTCAACTGCTACTTCCCCCTG |
| | | CAGAGCTACGGCTTCCAGCCCACCAACGGCGTGGGCTACCAGCCCTACCGGGTGGTGGTTCTGAGCTTCGAGCTGC |
| | | TGCACGCCCCTGCCACCGTGTGCGGCCCCAAGAAAAGCACCAACCTGGTGAAGAACAAGTGCGTGAATTTCAATTTT |
| | | AACGGACTCACCGGAACCGGCGTGCTGACCGAGAGCAACAAGAAGTTCCTGCCCTTCCAGCAATTCGGCCGGGATA |
| | | TTGCCGACACCACTGACGCCGTGCGGGACCCCCAGACCCTGGAGATCCTGGACATCACACCCTGTAGCTTCGGCGG |
| | | GGTGAGCGTGATCACCCCCGGCACCAACACCAGCAACCAGGTGGCCGTGCTGTACCAGGACGTGAACTGCACCGAG |
| | | GTGCCCGTGGCCATCCACGCCGACCAGCTGACCCCCACCTGGCGGGTGTACAGCACCGGCAGCAACGTGTTCCAGA |
| | | CCCGGGCCGGCTGCCTGATCGGCGCCGAGCACGTGAATAACAGCTACGAGTGCGACATCCCCATCGGAGCCGGAAT |
| | | CTGCGCCAGCTACCAGACCCAGACCAACAGCCCCCGGAGAGCCCGGAGCGTGGCCAGCCAGAGCATTATTGCCTAC |
| | | ACCATGAGCCTGGGCGCCGAGAACAGCGTGGCCTACAGCAACAATAGTATTGCCATCCCCACCAACTTCACCATCAG |
| | | CGTGACCACCGAAATCCTCCCCGTGAGCATGACCAAGACCAGCGTGGACTGCACCATGTACATCTGCGGCGACAGC |
| | | ACCGAGTGCAGCAACCTGCTCCTGCAGTACGGCAGCTTCTGCACCCAGCTGAACCGGGCCCTGACCGGAATTGCCG |
| | | TGGAGCAGGACAAGAACACCCAGGAGGTGTTCGCCCAGGTGAAGCAGATCTACAAGACACCTCCCATCAAGGACTTC |
| | | GGCGGCTTCAACTTCAGCCAGATCCTGCCCGACCCCAGCAAGCCCAGCAAGCGGAGCTTCATCGAGGACCTGTTGT |
| | | TCAACAAGGTGACCCTGGCCGACGCCGGCTTCATCAAGCAGTACGGCGACTGTCTGGGAGATATTGCCGCACGGGA |
| | | CCTGATCTGCGCCCAGAAGTTCAACGGCCTGACCGTGCTGCCCCCACTGCTGACCGACGAGATGATCGCCCAGTAC |
| | | ACAAGCGCTCTGCTCGCCGGCACCATCACCAGCGGCTGGACCTTCGGCGCCGGCGCTGCCCTGCAGATCCCCTTCG |
| | | CCATGCAGATGGCCTACCGGTTCAACGGCATCGGCGTGACCCAGAACGTGCTGTACGAGAACCAGAAGCTGATCGC |
| | | CAACCAGTTCAACAGCGCCATCGGCAAGATCCAGGACAGCCTGAGCTCCACCGCCAGCGCCCTGGGCAAGCTGCAG |
| | | GACGTGGTGAACCAGAACGCCCAGGCCCTGAACACCCTGGTGAAGCAGCTGAGCAGCAACTTCGGCGCCATCAGCT |
| | | CTGTGCTGAACGACATCCTGAGCCGGCTGGATCCTCCTGAGGCCGAGGTGCAAATCGATCGGCTCATCACCGGCCG |
| | | GCTGCAGAGCCTGCAGACCTACGTGACACAGCAGCTGATCCGGGCCGCCGAGATCCGGGCCAGCGCCAACCTGGC |
| | | CGCTACCAAGATGAGCGAGTGCGTGCTGGGCCAGAGCAAGCGGGTGGACTTCTGCGGCAAGGGCTACCACCTGATG |
| | | AGCTTCCCCCAGAGCGCCCCCCACGGCGTGGTGTTCCTGCACGTGACCTACGTGCCCGCCCAGGAGAAGAACTTCA |
| | | CCACCGCCCCCGCCATCTGCCACGACGGCAAGGCCCACTTCCCCCGGGAGGGCGTGTTCGTGAGCAACGGCACCC |
| | | ACTGGTTCGTGACCCAGCGGAACTTCTACGAGCCCCAGATCATAACCACAGACAACACCTTCGTGAGCGGCAACTGC |
| | | GACGTGGTCATCGGCATCGTGAACAACACCGTGTACGACCCCCTGCAGCCCGAGCTGGACAGCTTCAAGGAGGAAC |
| | | TGGACAAGTACTTCAAGAACCACACCAGCCCCGACGTGGACCTGGGCGACATCAGCGGCATCAACGCCAGCGTGGT |
| | | GAACATCCAGAAGGAGATCGACCGGCTGAACGAGGTGGCCAAGAACCTGAACGAGAGCCTGATCGACCTGCAGGAG |
| | | CTGGGCAAGTACGAGCAGTACATCAAGTGGCCCTGGTACATCTGGCTGGGCTTTATTGCCGGCCTGATCGCCATCGT |
| | | GATGGTGACCATCATGCTGTGCTGTATGACAAGCTGTTGCAGTTGCCTGAAGGGCTGCTGCAGCTGCGGCAGCTGCT |
| | | GCAAGTTCGACGAGGACGACAGCGAGCCCGTGCTGAAGGGCGTGAAGCTGCACTACACCTAAATGAGACCATA |
| 260 | 2A0681 | ATAGGTCTCACACACTAGGGCGCGCCAGTCCTCAAGCTGGGCCTGACACAACTGTGTTCACTAGCAACTACTCCCAG |
| | | ACACCATGGTACTCCCGACTCCTGAGGTACTCCCTGCCGTTACTGCCCTGTGGGGCAAGGTGATACTCCCTGAAGTT |
| | | GGTGGTGAGGCCCTGGGCAGGCTACTCCCGGTCTACCCTTGGACCCAGAGGTCGACTCGGACCGGCCACCATGTTT |
| | | GTTTTCTTAGTTTTATTACCTTTAGTTTCTTCCCAGTGTGTTAATTTAACTACTAGAACTCAATTACCTCCTGCTTATACT |
| | | AATTCTTTTACTAGAGGAGTGTATTATCCTGATAAGGTTTTTAGATCTTCTGTTCTGCACTCTACTCAGGATTTGTTCTT |
| | | GCCCTTCTTCTCTAACGTGACCTGGTTCCACGCTATTCACGTTTCTGGAACCAACGGCACTAAGAGATTTGATAATCC |
| | | CGTTTTGCCTTTTAACGACGGAGTTTATTTCGCCTCTACCGAAAAATCTAACATCATTAGGGGCTGGATCTTTGGAACT |
| | | ACCCTGGATTCTAAAACTCAATCTTTATTGATTGTGAATAACGCTACCAACGTTGTGATTAAAGTTTGTGAATTTCAATT |
| | | TTGTAACGATCCTTTTCTGGGAGTTTATTATCATAAAAACAATAAATCTTGGATGGAATCTGAATTCCGCGTTTATTCTT |
| | | CTGCCAATAACTGTACCTTTGAGTACGTTTCTCAACCTTTCTTAATGGATTTAGAAGGAAAACAAGGCAATTTCAAAAAC |
| | | TTAAGAGAATTTGTTTTTAAAAATATTGACGGATATTTTAAAATTTATTCTAAGCATACTCCTATTAATTTAGTGAGAGAC |
| | | TTACCCCAAGGATTTTCTGCTTTAGAACCTTTAGTTGACTTACCTATTGGAATCAATATCACTAGATTTCAAACTTTATTA |
| | | GCTTTACATAGATCTTATTTAACTCCTGGAGATTCTTCCTCCGGCTGGACAGCCGGAGCCGCTGCTTATTACGTTGGA |
| | | TATTTACAGCCTAGAACTTTTCTGCTGAAATATAACGAAAACGGAACCATTACCGACGCCGTTGACTGTGCTCTGGATC |
| | | CTTTATCTGAAACCAAGTGTACTTTAAAATCTTTTACCGTTGAAAAAGGAATTTATCAAACTTCTAATTTTAGAGTTCAAC |
| | | CTACTGAATCTATCGTTAGATTCCCTAATATTACTAATTTGTGTCCCTTTGGAGAGGTTTTCAACGCTACTAGATTTGCC |
| | | TCTGTTTACGCTTGGAATCGCAAGAGAATTTCTAATTGTGTGGCCGATTACTCCGTTTTGTATAACTCTGCTTCTTTCTC |
| | | TACTTTTAAGTGTTACGGAGTTTCCCCTACTAAACTGAACGATTTGTGTTTTACTAACGTTTACGCTGATTCTTTTGTTAT |
| | | TAGAGGCGACGAAGTTAGACAAATTGCTCCTGGACAAACTGGAAAGATTGCTGACTATAACTATAAACTGCCTGACGA |
| | | TTTTACCGGCTGTGTTATTGCTTGGAATTCTAATAATTTAGATTCTAAAGTTGGAGGAAATTATAATTATTTATATAGATT |
| | | ATTTAGAAAATCTAATTTAAAACCTTTTGAAAGAGATATTTCCACTGAGATTTACCAGGCTGGATCTACTCCTTGTAACG |
| | | GCGTTGAGGGATTTAATTGTTATTTTCCTTTACAATCTTACGGATTTCAACCTACCAACGGAGTTGGATATCAACCTTAC |
| | | AGAGTTGTGGTGCTGTCTTTTGAATTATTACACGCTCCCGCTACTGTTTGCGGCCCTAAAAAGTCTACTAATCTGGTTA |
| | | AGAATAAGTGTGTGAATTTTAATTTCAACGGATTGACCGGAACTGGAGTTCTGACTGAATCTAATAAAAAATTTTTACCC |
| | | TTTCAACAATTTGGAAGGGATATCGCTGATACTACTGACGCTGTTAGAGATCCTCAAACTTTAGAAATCCTGGATATTA |
| | | CTCCTTGTTCTTTTGGAGGAGTTTCCGTTATTACTCCTGGCACTAATACTTCTAATCAAGTTGCTGTTTTATATCAAGAC |
| | | GTTAATTGCACCGAAGTTCCTGTTGCTATTCACGCTGATCAGTTAACTCCTACTTGGAGAGTTTATTCTACTGGATCTA |
| | | ACGTTTTTCAAACCAGAGCTGGCTGTTTAATCGGAGCTGAACACGTGAATAATTCTTACGAGTGCGATATTCCCATCG |
| | | GAGCTGGAATTTGTGCCTCTTATCAAACTCAAACCAATTCTCCTAGGAGGGCTAGATCCGTTGCCTCTCAATCTATTAT |
| | | TGCTTATACCATGTCTTTAGGAGCTGAAAATTCCGTTGCTTACTCTAACAATTCTATTGCTATTCCTACTAACTTTACTAT |
| | | TTCTGTTACTACTGAAATTCTGCCCGTGTCCATGACTAAAACTTCTGTTGATTGTACTATGTATATTTGTGGAGATTCCA |
| | | CTGAGTGTTCTAACCTGTTATTACAATACGGATCTTTTTGTACTCAATTAAATAGAGCTCTGACTGGAATTGCTGTTGAA |
| | | CAGGACAAAAATACTCAAGAAGTTTTTGCCCAAGTTAAACAGATTTATAAAACTCCTCCTATTAAAGATTTTGGAGGATT |
| | | TAATTTCTCTCAAATTTTACCTGACCCTTCTAAGCCTTCCAAAAGGTCTTTCATCGAAGATTTATTATTTAATAAAGTTAC |
| | | CTTAGCCGACGCTGGATTTATTAAGCAATACGGAGATTGCTTAGGCGATATCGCTGCTAGAGACTTAATTTGTGCTCA |
| | | AAAATTTAACGGATTAACTGTTTTACCTCCCCTGTTAACTGACGAAATGATCGCTCAATATACTTCTGCTCTGTTAGCTG |
| | | GAACTATTACTTCTGGCTGGACTTTTGGAGCTGGAGCTGCTTTACAAATTCCCTTTGCTATGCAGATGGCTTATAGATT |
| | | TAACGGAATTGGAGTTACTCAAAACGTTTTATACGAAAATCAAAAATTAATTGCTAATCAATTTAATTCTGCTATTGGAA |
| | | AAATTCAAGATTCCCTGTCTTCTACTGCTTCCGCTCTGGGAAAATTACAAGACGTTGTTAATCAAAACGCTCAAGCTTT |
| | | GAATACCTTAGTTAAACAATTATCTTCCAATTTTGGAGCTATTTCTTCTGTTTTAAACGACATTTTATCTAGATTAGATCC |
| | | CCCTGAAGCTGAAGTTCAGATTGACAGATTGATTACCGGAAGATTACAATCTTTACAAACTTACGTTACTCAACAACTG |
| | | ATTAGAGCTGCTGAAATTAGAGCCTCTGCTAATTTAGCTGCTACTAAGATGTCTGAGTGTGTTTTAGGACAGTCCAAAA |
| | | GAGTTGATTTTTGTGGAAAAGGCTATCATCTGATGTCTTTTCCTCAATCTGCTCCTCACGGAGTTGTGTTTTTACACGTT |
| | | ACTTACGTTCCTGCTCAAGAGAAGAATTTTACTACTGCTCCTGCTATTTGTCACGACGGAAAAGCTCATTTTCCTAGAG |
| | | AAGGAGTTTTTGTTTCTAACGGAACTCATTGGTTTGTTACTCAAAGAAACTTTTACGAACCTCAGATTATTACCACTGAT |
| | | AATACCTTTGTTTCTGGCAATTGTGACGTGGTTATTGGAATTGTTAATAATACTGTTTACGATCCTTTACAACCCGAACT |
| | | GGATTCTTTTAAAGAAGAATTAGATAAATACTTTAAAAATCATACTTCTCCCGACGTTGATTTAGGAGATATCTCTGGAA |
| | | TTAACGCTTCTGTTGTGAATATTCAAAAGGAAATTGATAGATTAAACGAAGTGGCTAAAAATTTAAACGAGTCTTTAATT |
| | | GATTTACAGGAACTGGGAAAATACGAACAATATATTAAGTGGCCTTGGTACATTTGGTTAGGCTTTATTGCTGGACTGA |
| | | TTGCTATTGTGATGGTTACTATCATGTTGTGTTGCATGACTTCTTGTTGTTCCTGTTTAAAAGGCTGTTGCTCTTGTGGA |
| | | TCTTGTTGCAAATTTGACGAAGACGATTCTGAACCTGTTCTGAAAGGAGTTAAACTGCATTATACTTAAATGAGACCAT |
| | | A |
| 261 | 2A1681 | ATAGGTCTCACACACTAGGGCGCGCCAGTCCTCAAGCTGGGCCTGACACAACTGTGTTCACTAGCAACTACTCCCAG |
| | | ACACCATGGTACTCCCGACTCCTGAGGTACTCCCTGCCGTTACTGCCCTGTGGGGCAAGGTGATACTCCCTGAAGTT |
| | | GGTGGTGAGGCCCTGGGCAGGCTACTCCCGGTCTACCCTTGGACCCAGAGGTCGACTCGGACCGGCCACCATGTTC |
| | | GTGTTCTTAGTGCTGTTACCCCTGGTTTCTTCTCAGTGTGTCAATTTAACCACCAGAACCCAACTGCCTCCTGCCTATA |
| | | CTAATTCCTTTACCCGCGGAGTTTACTATCCCGATAAGGTTTTTCGCTCCTCTGTTTTACACTCTACTCAGGACTTATTC |
| | | TTACCCTTCTTCTCCAACGTTACTTGGTTTCACGCCATTCACGTTTCTGGAACCAACGGAACTAAGAGATTCGATAATC |
| | | CTGTGTTACCTTTCAACGACGGAGTTTATTTTGCTTCCACTGAAAAATCCAATATCATTAGGGGCTGGATCTTTGGAAC |
| | | CACTTTAGATTCCAAGACTCAATCTTTATTAATTGTGAATAACGCCACCAACGTGGTTATCAAAGTGTGTGAATTCCAAT |
| | | TTTGTAACGACCCTTTCTTAGGCGTGTATTATCACAAAAATAATAAATCTTGGATGGAATCTGAATTTCGCGTTTACTCT |
| | | TCCGCTAACAACTGCACTTTTGAGTACGTTTCCCAGCCTTTTCTGATGGATCTGGAAGGCAAACAGGGAAATTTTAAAA |
| | | ATCTGAGAGAATTTGTTTTTAAGAACATTGACGGATATTTCAAGATCTATTCTAAACACACTCCCATCAATTTGGTTAGA |
| | | GATTTACCTCAGGGCTTTTCTGCTTTAGAACCTTTAGTTGACCTGCCTATTGGAATCAACATTACTAGATTTCAAACTCT |
| | | GTTAGCTCTGCATAGATCTTATTTAACTCCTGGAGATTCCTCCTCCGGCTGGACCGCCGGAGCTGCTGCTTATTACGT |
| | | TGGCTATTTACAACCCAGAACTTTTCTGTTAAAATATAACGAAAACGGCACCATCACTGACGCCGTGGATTGTGCTTTA |
| | | GATCCCTTATCCGAAACTAAGTGTACTCTGAAATCCTTCACTGTTGAAAAAGGAATTTATCAGACCTCTAATTTTAGAGT |
| | | GCAACCTACCGAATCTATTGTTAGATTTCCTAATATTACCAACTTGTGCCCTTTTGGCGAAGTTTTCAACGCTACTAGAT |
| | | TTGCTTCTGTTTACGCTTGGAATAGAAAAAGAATTTCTAACTGTGTGGCTGACTATTCCGTGTTATACAATTCTGCCTCT |
| | | TTTTCTACTTTTAAGTGTTACGGAGTGTCTCCCACTAAACTGAACGATTTGTGCTTTACCAACGTTTACGCTGATTCTTT |
| | | TGTTATTAGAGGCGACGAGGTTCGCCAGATTGCCCCTGGCCAGACTGGCAAAATTGCTGACTATAACTATAAATTACC |
| | | TGACGATTTTACCGGCTGCGTTATCGCTTGGAACTCTAATAATCTGGATTCTAAAGTTGGAGGAAACTACAATTATTTA |
| | | TATAGATTATTTAGAAAATCTAATTTAAAACCCTTCGAGAGGGATATCTCTACTGAAATCTACCAAGCTGGATCTACTCC |
| | | TTGCAACGGCGTTGAAGGATTTAATTGTTATTTTCCTTTACAATCTTACGGATTCCAACCTACTAACGGAGTTGGATATC |
| | | AGCCTTATAGAGTGGTGGTTTTATCTTTCGAATTATTACACGCTCCCGCTACTGTGTGTGGACCCAAAAAGTCTACTAA |
| | | TTTAGTTAAAAATAAGTGTGTTAATTTCAATTTTAACGGACTCACTGGCACTGGAGTGTTAACTGAATCTAATAAAAAGT |
| | | TCTTACCTTTCCAACAGTTCGGACGCGACATTGCCGACACCACTGACGCTGTGAGAGATCCTCAAACCTTAGAGATTC |
| | | TGGATATTACCCCTTGTTCCTTTGGAGGCGTTTCCGTTATCACTCCCGGAACTAACACTTCTAATCAGGTTGCCGTTTT |
| | | ATATCAGGACGTTAATTGCACTGAAGTTCCTGTTGCTATTCACGCTGATCAACTGACTCCTACTTGGAGAGTTTATTCC |
| | | ACTGGATCTAACGTTTTTCAGACTCGCGCCGGCTGTTTAATTGGAGCTGAACACGTGAACAACTCCTACGAGTGCGAT |
| | | ATTCCTATTGGCGCTGGCATCTGTGCTTCTTATCAAACTCAAACTAATTCTCCCAGGAGGGCTCGCTCTGTTGCTTCCC |
| | | AATCTATCATTGCTTATACTATGTCCTTAGGCGCTGAAAATTCTGTTGCTTATTCCAACAATTCTATTGCTATTCCCACC |
| | | AATTTTACTATTTCTGTGACTACTGAAATTTTACCTGTTTCCATGACTAAGACTTCTGTTGATTGTACTATGTATATCTGC |
| | | GGAGATTCTACTGAGTGTTCCAACCTCTTATTACAGTACGGATCTTTTTGCACTCAGTTAAATAGAGCTCTGACTGGAA |
| | | TCGCTGTTGAACAAGACAAAAACACCCAAGAAGTGTTTGCTCAGGTGAAGCAAATTTACAAGACACCTCCTATTAAGG |
| | | ATTTTGGAGGATTCAATTTTTCCCAGATTTTACCTGATCCTTCTAAACCTTCTAAACGCTCCTTTATTGAAGATTTACTGT |
| | | TCAATAAAGTGACTCTGGCTGACGCTGGCTTCATCAAGCAATACGGCGATTGTTTAGGAGACATCGCTGCTAGAGACT |
| | | TAATTTGTGCTCAAAAATTCAACGGACTGACTGTTCTGCCCCCTTTACTGACCGACGAGATGATCGCCCAATATACTTC |
| | | TGCCCTGTTAGCTGGCACCATTACTTCTGGCTGGACCTTTGGAGCTGGCGCTGCTTTACAAATTCCTTTCGCCATGCA |
| | | GATGGCTTATCGCTTTAACGGAATTGGCGTTACCCAAAACGTGTTATACGAGAATCAAAAATTAATCGCCAATCAATTT |
| | | AATTCTGCTATTGGCAAGATTCAAGATTCTTTATCTTCTACTGCTTCCGCCCTGGGCAAACTGCAGGACGTTGTTAACC |
| | | AGAACGCTCAAGCCCTGAACACTTTAGTGAAGCAACTGTCTTCTAACTTCGGCGCTATTTCTTCCGTTTTAAACGACAT |
| | | TCTGTCTCGCTTAGATCCCCCTGAGGCTGAGGTTCAAATTGATAGACTGATTACTGGAAGATTACAGTCCCTGCAAAC |
| | | TTACGTTACTCAACAGTTAATCAGAGCTGCTGAAATCCGCGCTTCCGCTAATTTAGCTGCTACTAAAATGTCTGAGTGT |
| | | GTGTTAGGCCAGTCTAAGAGAGTTGACTTTTGTGGCAAGGGCTATCATTTAATGTCTTTTCCTCAGTCCGCTCCTCAC |
| | | GGCGTTGTGTTCTTACACGTGACTTACGTTCCCGCCCAAGAGAAAAATTTTACCACTGCTCCTGCTATTTGTCACGAC |
| | | GGAAAAGCTCATTTTCCTAGAGAAGGAGTGTTTGTTTCTAACGGAACCCACTGGTTTGTGACTCAAAGAAACTTTTACG |
| | | AACCCCAAATTATTACTACTGACAATACTTTCGTTTCCGGCAACTGTGACGTGGTGATCGGAATTGTTAATAACACCGT |
| | | TTACGATCCCTTACAACCTGAATTAGATTCTTTTAAAGAAGAATTAGATAAATATTTCAAAAATCATACTTCCCCTGACG |
| | | TTGATCTGGGAGATATTTCTGGAATTAACGCTTCTGTGGTGAACATTCAAAAAGAGATTGACAGATTAAACGAAGTTGC |
| | | TAAAAACCTGAACGAGTCCCTGATCGACCTGCAGGAACTGGGAAAATACGAGCAGTATATTAAGTGGCCTTGGTATAT |
| | | TTGGTTAGGATTTATTGCTGGATTAATTGCTATTGTGATGGTGACCATTATGTTGTGCTGTATGACCTCTTGTTGTTCTT |
| | | GCCTGAAGGGCTGCTGTTCTTGTGGCTCTTGTTGCAAGTTTGACGAAGACGATTCCGAACCCGTTTTAAAGGGAGTGA |
| | | AACTGCATTATACTTAAATGAGACCATA |
| 262 | 2A2681 | ATAGGTCTCACACACTAGGGCGCGCCAGTCCTCAAGCTGGGCCTGACACAACTGTGTTCACTAGCAACTACTCCCAG |
| | | ACACCATGGTACTCCCGACTCCTGAGGTACTCCCTGCCGTTACTGCCCTGTGGGGCAAGGTGATACTCCCTGAAGTT |
| | | GGTGGTGAGGCCCTGGGCAGGCTACTCCCGGTCTACCCTTGGACCCAGAGGTCGACTCGGACCGGCCACCATGTTT |
| | | GTTTTTCTGGTTTTATTACCCTTAGTGTCCTCCCAGTGCGTGAACTTAACTACCCGCACTCAGCTGCCTCCTGCCTATA |
| | | CCAACTCTTTCACTAGAGGCGTTTACTATCCTGACAAAGTTTTTAGATCTTCTGTGCTGCATTCCACTCAAGATTTATTC |
| | | CTGCCCTTCTTTTCTAACGTTACTTGGTTTCACGCCATCCACGTGTCTGGCACCAACGGAACCAAACGCTTTGACAAC |
| | | CCTGTGTTACCTTTTAACGACGGAGTTTACTTCGCCTCCACTGAAAAGTCCAATATTATCCGGGGCTGGATTTTCGGAA |
| | | CCACCTTAGATTCTAAAACCCAGTCTCTGCTGATTGTGAACAACGCCACTAACGTGGTTATTAAAGTGTGCGAGTTTCA |
| | | ATTTTGCAACGACCCTTTTCTGGGAGTTTATTACCACAAAAATAACAAATCTTGGATGGAGTCTGAATTTAGAGTGTATT |
| | | CTTCCGCCAATAATTGCACCTTCGAGTACGTTTCCCAACCCTTTCTGATGGACTTAGAAGGAAAGCAAGGCAATTTCAA |
| | | AAACCTGCGCGAATTTGTGTTCAAAAATATTGACGGCTATTTCAAAATTTACTCCAAGCATACCCCTATTAATTTAGTGC |
| | | GCGATCTGCCTCAGGGCTTCTCTGCCTTAGAGCCTTTAGTGGACCTGCCTATTGGCATTAATATTACTCGCTTTCAGA |
| | | CTTTACTGGCCCTGCATAGATCTTATTTAACTCCTGGAGACTCTTCTTCCGGCTGGACCGCTGGAGCCGCTGCCTACT |
| | | ACGTTGGCTATCTGCAGCCTAGAACTTTTTTATTAAAGTACAACGAAAACGGAACCATCACTGACGCTGTTGATTGCGC |
| | | CCTGGATCCTTTATCTGAAACAAAGTGCACCCTGAAGTCCTTCACCGTTGAAAAGGGCATTTACCAAACATCCAATTTT |
| | | AGGGTTCAGCCTACTGAATCCATCGTGCGCTTTCCCAACATCACTAACTTGTGTCCCTTTGGCGAGGTGTTTAACGCC |
| | | ACTCGCTTTGCCTCTGTGTACGCCTGGAACAGAAAGAGAATTTCCAATTGCGTGGCTGATTATTCTGTGCTGTATAACT |
| | | CCGCTTCTTTCTCCACCTTTAAGTGTTACGGAGTGTCCCCTACTAAACTGAACGACTTGTGCTTCACCAACGTTTACGC |
| | | CGATTCTTTTGTGATTCGCGGCGACGAAGTTAGACAAATCGCCCCCGGACAGACCGGAAAGATCGCTGATTACAACT |
| | | ATAAGTTACCTGACGACTTCACCGGCTGCGTGATTGCTTGGAATTCTAATAACTTAGACTCTAAAGTTGGAGGCAACTA |
| | | CAATTACCTGTATAGATTATTCCGCAAATCTAACCTGAAACCTTTTGAGAGAGACATCTCCACCGAAATTTACCAAGCC |
| | | GGCTCTACCCCCTGCAACGGAGTTGAAGGATTTAATTGCTACTTTCCCCTGCAGTCCTACGGATTCCAACCTACTAAC |
| | | GGCGTTGGATACCAACCTTACAGAGTTGTGGTGCTGTCTTTTGAATTATTACACGCCCCTGCCACCGTTTGTGGCCCT |
| | | AAGAAATCTACCAATCTGGTTAAAAACAAGTGTGTGAATTTTAATTTTAACGGCCTGACCGGCACTGGCGTTTTAACCG |
| | | AGTCTAACAAGAAGTTTCTGCCTTTCCAACAATTTGGCAGAGACATCGCCGACACCACCGACGCTGTGCGCGATCCTC |
| | | AAACCTTAGAAATTTTAGATATTACTCCTTGCTCTTTCGGCGGCGTTTCCGTTATTACTCCTGGAACCAATACCTCTAAT |
| | | CAAGTGGCTGTGCTGTACCAGGACGTTAATTGCACTGAAGTGCCTGTGGCTATTCACGCCGATCAACTGACCCCCAC |
| | | TTGGAGAGTTTATTCTACTGGATCTAACGTGTTCCAAACTAGAGCTGGCTGTCTGATCGGCGCCGAGCACGTTAATAA |
| | | TTCTTACGAGTGTGATATCCCTATTGGCGCTGGCATTTGCGCCTCTTACCAGACCCAGACCAACTCCCCCCGCAGAG |
| | | CTAGATCCGTGGCCTCTCAGTCCATTATTGCCTATACTATGTCCTTAGGAGCCGAAAATTCTGTGGCTTACTCCAACAA |
| | | TTCTATTGCCATTCCCACTAATTTTACTATCTCTGTGACTACCGAAATCCTGCCCGTGTCCATGACTAAGACCTCCGTG |
| | | GACTGCACCATGTATATCTGCGGAGACTCTACTGAGTGTTCCAACTTATTACTGCAGTACGGCTCTTTCTGTACTCAGC |
| | | TGAATAGAGCCCTGACTGGAATTGCTGTGGAGCAAGACAAAAATACCCAGGAGGTGTTCGCTCAGGTGAAACAAATTT |
| | | ATAAGACCCCTCCCATCAAAGATTTTGGAGGCTTCAATTTTTCTCAAATTCTGCCCGACCCCTCCAAGCCTTCCAAAAG |
| | | ATCCTTTATTGAAGATCTGCTGTTCAATAAGGTTACTTTAGCCGACGCCGGCTTCATTAAGCAGTACGGAGATTGTCTG |
| | | GGCGATATTGCTGCCCGCGACCTGATTTGCGCTCAGAAGTTCAACGGATTAACCGTTCTGCCTCCTCTGCTGACTGA |
| | | CGAGATGATTGCTCAGTATACTTCTGCCCTGCTGGCTGGAACTATTACCTCTGGCTGGACCTTCGGCGCCGGAGCTG |
| | | CTCTGCAAATTCCCTTCGCCATGCAAATGGCTTACAGATTTAACGGCATCGGAGTTACTCAAAACGTGCTGTACGAAA |
| | | ACCAAAAATTAATTGCCAACCAGTTCAATTCCGCTATCGGCAAAATTCAGGATTCTCTGTCTTCTACTGCTTCCGCTTTA |
| | | GGCAAACTGCAAGACGTTGTTAACCAGAACGCCCAAGCTTTAAACACCCTGGTTAAGCAGCTGTCCTCTAATTTTGGC |
| | | GCTATCTCTTCTGTTCTGAACGACATTTTATCTAGACTGGACCCCCCTGAAGCCGAGGTGCAGATTGATAGATTAATCA |
| | | CCGGAAGACTGCAATCCCTGCAGACCTACGTTACCCAGCAATTAATCAGAGCCGCCGAGATCAGAGCCTCTGCCAAC |
| | | TTAGCTGCCACCAAAATGTCCGAGTGTGTGCTGGGACAGTCCAAGCGCGTGGACTTCTGTGGAAAGGGATACCACCT |
| | | GATGTCTTTTCCTCAATCTGCTCCCCACGGAGTTGTGTTTCTGCACGTTACTTACGTGCCTGCCCAAGAGAAAAACTTT |
| | | ACTACCGCCCCCGCTATTTGCCACGACGGCAAGGCTCACTTCCCTAGAGAAGGAGTTTTTGTGTCTAACGGAACCCA |
| | | TTGGTTTGTGACTCAAAGAAATTTTTACGAGCCTCAAATCATTACCACCGATAACACTTTTGTTTCCGGCAATTGTGAC |
| | | GTGGTGATTGGAATCGTTAACAACACCGTTTACGACCCTTTACAGCCCGAATTAGACTCTTTTAAAGAGGAGTTAGATA |
| | | AATATTTTAAAAACCATACCTCTCCCGACGTGGACCTGGGAGATATTTCCGGAATTAACGCTTCTGTTGTGAATATCCA |
| | | GAAAGAGATCGACAGATTAAACGAGGTTGCTAAAAATTTAAACGAATCCCTGATTGACCTGCAGGAGCTGGGCAAATA |
| | | CGAACAATATATTAAGTGGCCTTGGTATATTTGGTTAGGCTTCATCGCTGGATTAATCGCTATTGTTATGGTGACTATTA |
| | | TGTTGTGTTGTATGACTTCCTGCTGTTCTTGCTTAAAAGGCTGCTGTTCCTGCGGATCCTGTTGTAAATTCGACGAGGA |
| | | CGATTCCGAGCCTGTTTTAAAAGGAGTGAAATTACATTATACTTAAATGAGACCATA |
| 263 | 2A3681 | ATAGGTCTCACACACTAGGGCGCGCCAGTCCTCAAGCTGGGCCTGACACAACTGTGTTCACTAGCAACTACTCCCAG |
| | | ACACCATGGTACTCCCGACTCCTGAGGTACTCCCTGCCGTTACTGCCCTGTGGGGCAAGGTGATACTCCCTGAAGTT |
| | | GGTGGTGAGGCCCTGGGCAGGCTACTCCCGGTCTACCCTTGGACCCAGAGGTCGACTCGGACCGGCCACCATGTTC |
| | | GTGTTCCTGGTGCTGCTGCCCTTAGTGTCCTCCCAGTGTGTGAACCTGACCACCAGAACCCAGTTACCTCCCGCTTA |
| | | CACCAACTCCTTTACCCGCGGAGTGTACTACCCTGATAAGGTTTTTAGATCCTCCGTTCTGCATTCTACCCAAGACCT |
| | | GTTTTTACCCTTCTTTTCCAACGTGACCTGGTTCCACGCTATCCACGTGTCCGGCACTAACGGCACTAAGCGCTTCGA |
| | | CAATCCTGTGCTCCCTTTTAACGACGGCGTGTACTTCGCTTCTACCGAAAAATCCAACATCATCCGCGGCTGGATCTT |
| | | CGGAACTACCCTGGATTCTAAAACCCAGTCTTTATTAATCGTGAATAACGCCACCAACGTGGTTATTAAGGTCTGTGAG |
| | | TTCCAATTTTGCAACGACCCTTTCTTAGGCGTTTATTACCACAAAAACAATAAGTCTTGGATGGAGTCCGAATTCCGCG |
| | | TGTACTCCTCTGCCAACAATTGCACCTTTGAGTACGTGTCCCAGCCCTTCCTGATGGACCTGGAAGGCAAGCAGGGC |
| | | AATTTTAAAAATCTGCGCGAATTCGTTTTCAAGAACATCGACGGCTACTTTAAGATTTATTCCAAACATACCCCCATCAA |
| | | CCTGGTGCGCGATTTACCCCAGGGCTTCTCCGCCTTAGAGCCCTTAGTGGATCTGCCTATCGGCATCAATATCACCC |
| | | GCTTTCAGACTCTGCTGGCCCTGCACAGATCCTACCTGACTCCCGGAGACTCTTCTTCTGGCTGGACCGCCGGAGCT |
| | | GCCGCCTACTACGTGGGCTACCTGCAACCCCGCACCTTTCTGCTGAAGTACAACGAAAACGGCACCATCACAGACGC |
| | | CGTTGACTGCGCTCTGGACCCCCTGTCTGAGACTAAGTGCACCCTGAAGTCCTTCACTGTGGAGAAAGGAATCTACC |
| | | AGACCTCCAACTTCCGCGTGCAGCCTACCGAATCCATTGTTCGCTTCCCTAACATTACTAATCTGTGCCCCTTCGGAG |
| | | AAGTTTTCAACGCCACTAGATTCGCTTCCGTGTACGCCTGGAATCGCAAGCGCATCTCCAATTGCGTTGCTGACTACT |
| | | CTGTGTTATACAATTCCGCCTCTTTTTCCACTTTCAAGTGCTACGGCGTGTCTCCTACCAAGCTGAACGACCTGTGTTT |
| | | CACTAACGTGTACGCTGATTCCTTTGTTATCCGCGGCGACGAGGTGAGACAGATCGCCCCTGGCCAGACCGGAAAAA |
| | | TCGCCGACTACAACTACAAGTTACCCGACGATTTTACCGGCTGTGTTATTGCTTGGAACTCTAATAATCTGGACTCTAA |
| | | AGTGGGCGGCAATTACAACTACTTATATCGCCTGTTCAGAAAATCCAACCTGAAACCCTTCGAACGGGACATTTCCAC |
| | | CGAAATTTATCAGGCCGGCTCCACCCCCTGCAACGGAGTGGAGGGCTTCAATTGCTACTTCCCCCTGCAATCCTACG |
| | | GCTTTCAGCCCACCAACGGCGTGGGATACCAGCCCTACCGCGTTGTTGTGTTATCCTTTGAACTGCTGCACGCTCCT |
| | | GCTACTGTGTGCGGACCTAAAAAATCCACCAATCTGGTGAAGAACAAGTGTGTGAATTTTAATTTTAACGGCCTGACC |
| | | GGCACCGGAGTGCTGACCGAGTCCAACAAAAAGTTTCTGCCCTTTCAGCAGTTCGGCCGGGACATTGCCGACACTAC |
| | | CGACGCTGTGAGGGATCCTCAGACCCTGGAGATTCTGGACATTACCCCTTGCTCCTTCGGAGGAGTTTCCGTGATCA |
| | | CCCCTGGCACCAATACTTCTAACCAAGTGGCTGTTTTATACCAGGACGTGAATTGTACTGAGGTGCCTGTGGCCATCC |
| | | ACGCCGATCAATTAACTCCCACCTGGAGAGTGTATTCCACCGGCTCTAACGTTTTCCAAACCCGCGCTGGCTGCTTAA |
| | | TCGGCGCTGAGCACGTGAACAACTCCTACGAGTGTGATATTCCTATCGGAGCCGGCATCTGTGCTTCCTACCAGACT |
| | | CAGACCAACTCCCCCAGACGCGCTAGGTCCGTGGCTTCCCAGTCTATTATCGCTTATACTATGTCCCTGGGAGCCGA |
| | | GAATTCTGTTGCCTATTCCAATAACTCCATCGCCATTCCTACCAACTTTACCATCTCTGTGACCACCGAGATTCTGCCC |
| | | GTGTCCATGACCAAAACTTCTGTGGATTGTACCATGTACATCTGTGGAGATTCCACCGAGTGCTCTAACCTGCTGCTG |
| | | CAGTACGGCTCCTTTTGCACCCAATTAAACCGCGCTCTGACTGGCATCGCCGTGGAGCAAGATAAAAACACCCAAGA |
| | | GGTGTTCGCTCAGGTGAAGCAGATCTACAAGACACCTCCCATTAAAGATTTCGGAGGCTTTAATTTCTCTCAGATCCT |
| | | GCCCGACCCTTCTAAGCCCTCTAAACGCTCCTTCATCGAGGATCTGCTGTTCAACAAGGTTACCCTGGCCGACGCTG |
| | | GCTTTATCAAACAGTACGGCGACTGCTTAGGCGACATCGCCGCCCGCGATTTAATTTGCGCTCAGAAGTTCAACGGA |
| | | CTGACCGTGCTGCCTCCTCTGCTGACCGACGAAATGATCGCCCAATACACCTCCGCCCTGCTGGCTGGCACTATCAC |
| | | CTCCGGCTGGACCTTTGGCGCTGGAGCCGCTCTGCAAATCCCTTTCGCTATGCAGATGGCTTACCGCTTTAACGGAA |
| | | TCGGCGTGACCCAGAACGTGTTATACGAAAATCAGAAATTAATTGCCAATCAATTTAACTCTGCCATCGGCAAGATTCA |
| | | GGATTCTCTGTCTTCTACTGCCTCTGCCTTAGGAAAACTGCAGGACGTGGTGAATCAGAACGCCCAGGCCCTGAATA |
| | | CCCTGGTTAAGCAACTGTCTTCCAATTTCGGCGCTATCTCCTCTGTGTTAAACGACATTCTGTCCCGCCTGGATCCTC |
| | | CTGAGGCCGAAGTGCAAATTGACCGCTTAATCACTGGCAGACTGCAATCCCTGCAAACCTACGTTACTCAACAGCTGA |
| | | TCAGAGCCGCCGAAATCCGCGCTTCTGCCAACTTAGCTGCTACCAAGATGTCCGAGTGCGTGCTGGGACAGTCCAAA |
| | | AGAGTTGACTTTTGCGGAAAGGGCTACCACCTGATGTCCTTTCCCCAGTCCGCCCCCCACGGAGTTGTTTTCCTGCA |
| | | CGTTACTTACGTTCCTGCTCAAGAGAAGAACTTCACCACCGCTCCCGCTATTTGCCACGACGGCAAGGCTCATTTCCC |
| | | CAGAGAGGGAGTGTTTGTGTCCAACGGCACTCACTGGTTTGTTACTCAGCGCAACTTCTACGAACCTCAGATCATCAC |
| | | CACTGACAATACCTTTGTGTCCGGAAACTGTGACGTGGTTATCGGCATTGTGAACAATACTGTGTACGACCCTTTACA |
| | | ACCTGAGCTGGATTCTTTTAAAGAAGAACTGGACAAGTACTTCAAGAACCACACCTCCCCCGACGTGGATCTGGGCG |
| | | ACATCTCCGGCATCAACGCCTCTGTGGTGAATATTCAGAAGGAGATTGATCGCCTGAACGAGGTTGCCAAGAATCTGA |
| | | ACGAGTCTCTGATCGACCTGCAGGAGCTGGGCAAGTACGAACAGTACATCAAGTGGCCCTGGTACATTTGGCTGGGC |
| | | TTTATTGCTGGACTGATCGCTATCGTTATGGTTACCATCATGCTGTGCTGTATGACCTCTTGCTGCTCCTGTCTGAAAG |
| | | GCTGTTGCTCCTGCGGCTCCTGTTGTAAGTTTGACGAGGACGACTCCGAGCCTGTTCTGAAAGGAGTTAAACTGCAC |
| | | TACACCTAAATGAGACCATA |
| 264 | 2A4681 | ATAGGTCTCACACACTAGGGCGCGCCAGTCCTCAAGCTGGGCCTGACACAACTGTGTTCACTAGCAACTACTCCCAG |
| | | ACACCATGGTACTCCCGACTCCTGAGGTACTCCCTGCCGTTACTGCCCTGTGGGGCAAGGTGATACTCCCTGAAGTT |
| | | GGTGGTGAGGCCCTGGGCAGGCTACTCCCGGTCTACCCTTGGACCCAGAGGTCGACTCGGACCGGCCACCATGTTT |
| | | GTGTTTCTGGTGCTGCTGCCTCTGGTGTCCTCTCAGTGTGTTAACCTGACCACCAGAACCCAACTGCCCCCCGCCTA |
| | | CACTAACTCTTTCACTCGCGGCGTGTACTACCCCGATAAAGTTTTCAGATCCTCCGTGCTCCATTCTACCCAAGACCT |
| | | GTTCTTACCCTTTTTCTCCAACGTGACCTGGTTCCACGCCATCCACGTGTCCGGCACTAACGGAACCAAGCGCTTCGA |
| | | TAATCCCGTGCTGCCTTTCAACGACGGAGTTTACTTTGCTTCCACCGAGAAGTCCAATATTATCCGGGGCTGGATCTT |
| | | CGGCACCACACTGGATTCCAAAACCCAGTCCTTACTGATTGTGAACAACGCCACTAACGTGGTGATCAAGGTGTGTGA |
| | | GTTCCAGTTCTGTAACGACCCCTTTCTGGGCGTGTATTATCATAAAAACAACAAGTCCTGGATGGAGTCCGAGTTTAG |
| | | AGTGTATTCTTCCGCTAACAACTGCACCTTCGAATACGTGTCCCAGCCTTTCCTGATGGATCTGGAAGGAAAGCAGGG |
| | | CAACTTCAAGAACTTACGCGAGTTCGTGTTCAAGAACATCGACGGCTACTTCAAAATTTACTCCAAGCACACCCCTATC |
| | | AACTTAGTGAGAGATCTGCCCCAGGGCTTCTCCGCTCTGGAACCTCTGGTGGACTTACCCATTGGCATCAACATCACT |
| | | CGCTTCCAAACACTGCTCGCTCTGCACCGCTCTTATCTGACCCCCGGCGACTCCTCTTCTGGCTGGACCGCCGGCGC |
| | | CGCTGCCTATTACGTGGGATACCTGCAACCCCGCACCTTTCTGCTGAAATACAACGAAAACGGCACCATCACAGACG |
| | | CTGTGGACTGCGCTCTGGACCCCCTGTCCGAGACTAAGTGCACCCTGAAGTCCTTCACCGTTGAAAAGGGCATTTAT |
| | | CAGACTTCCAACTTCCGCGTGCAGCCTACTGAGTCCATTGTGCGCTTCCCTAACATCACTAACTTGTGTCCTTTCGGC |
| | | GAAGTGTTCAACGCTACTCGCTTCGCCTCCGTGTACGCCTGGAACCGCAAGAGAATCTCCAATTGCGTGGCCGACTA |
| | | CTCTGTTCTCTACAACTCTGCCTCCTTCTCCACCTTTAAGTGCTACGGCGTGTCCCCCACCAAACTGAACGACCTGTG |
| | | CTTCACCAACGTGTACGCCGACTCCTTTGTGATCAGAGGCGACGAGGTGCGCCAGATCGCCCCCGGCCAGACTGGC |
| | | AAGATCGCCGATTACAATTACAAGCTGCCTGACGACTTTACCGGCTGCGTGATTGCCTGGAACTCCAACAACCTGGAC |
| | | TCCAAAGTGGGCGGCAACTACAACTACCTGTACCGCCTGTTTAGAAAATCCAACCTGAAGCCCTTCGAGCGGGACAT |
| | | TTCTACTGAGATCTACCAGGCCGGCTCCACCCCCTGTAACGGCGTGGAAGGCTTCAACTGCTACTTTCCCCTGCAGT |
| | | CCTACGGCTTCCAGCCTACCAACGGCGTGGGCTACCAGCCCTACCGCGTGGTGGTGTTATCCTTCGAGCTGCTGCA |
| | | CGCCCCTGCCACCGTGTGCGGACCCAAAAAGTCTACTAACCTGGTTAAGAATAAGTGTGTTAATTTCAATTTTAACGG |
| | | CTTAACCGGAACCGGAGTGCTGACCGAGTCCAATAAAAAGTTCCTGCCCTTTCAGCAGTTTGGCCGCGACATCGCCG |
| | | ATACTACCGACGCCGTGCGCGATCCCCAGACCCTGGAGATCCTGGACATTACCCCCTGCTCTTTCGGCGGAGTGTCC |
| | | GTGATCACCCCCGGCACCAATACCTCCAATCAGGTGGCCGTTCTGTACCAGGACGTGAATTGCACCGAAGTGCCCGT |
| | | GGCCATTCACGCCGACCAGTTAACTCCTACTTGGAGAGTGTACTCCACCGGCTCTAACGTGTTTCAGACCCGCGCCG |
| | | GCTGTCTGATCGGCGCCGAGCACGTGAACAACTCCTACGAGTGCGACATTCCCATTGGCGCCGGCATCTGCGCCTC |
| | | CTACCAAACCCAGACTAACTCTCCCAGACGCGCCCGCTCTGTGGCCTCTCAGTCCATCATCGCTTACACTATGTCCCT |
| | | GGGCGCCGAGAACTCCGTGGCCTATTCCAACAACTCCATCGCCATCCCCACTAACTTCACCATCTCTGTGACCACCG |
| | | AAATCCTCCCCGTGTCTATGACTAAGACTTCCGTGGACTGTACCATGTACATTTGCGGCGACTCCACCGAGTGTTCCA |
| | | ATCTGCTGCTGCAGTACGGCTCCTTTTGCACCCAGCTGAACCGCGCCCTGACCGGCATCGCTGTTGAGCAGGACAAG |
| | | AATACCCAGGAGGTGTTTGCTCAGGTGAAGCAAATCTATAAGACCCCTCCCATCAAGGACTTTGGCGGCTTCAACTTT |
| | | TCCCAGATCCTGCCCGACCCTTCCAAGCCCTCCAAACGCTCTTTCATCGAAGATCTGCTGTTCAATAAAGTGACCCTG |
| | | GCCGACGCCGGCTTCATCAAGCAGTACGGAGACTGTCTGGGAGACATTGCTGCTCGCGACCTGATTTGCGCCCAGA |
| | | AGTTCAACGGCCTGACCGTGCTGCCCCCTCTGCTGACCGACGAGATGATCGCTCAGTACACCTCCGCCCTGCTGGCT |
| | | GGCACTATCACCTCCGGCTGGACTTTCGGCGCTGGCGCCGCCCTGCAGATTCCCTTCGCCATGCAGATGGCTTATCG |
| | | CTTCAACGGAATCGGAGTGACACAGAACGTGCTGTACGAGAATCAGAAACTGATCGCTAACCAGTTTAATTCCGCCAT |
| | | TGGCAAGATCCAGGACTCCCTGTCCTCTACTGCTTCCGCCCTGGGCAAGCTGCAGGACGTGGTGAACCAGAACGCC |
| | | CAGGCTCTGAACACCCTGGTGAAGCAACTGTCTTCTAACTTTGGAGCCATTTCTTCCGTGCTGAACGACATCCTGTCC |
| | | AGACTGGATCCTCCTGAGGCCGAGGTGCAGATCGACCGCCTGATCACTGGCCGCCTGCAGTCCCTGCAGACTTACG |
| | | TTACCCAGCAATTAATCCGCGCTGCCGAGATCCGCGCCTCCGCCAACCTGGCCGCCACCAAGATGTCCGAGTGCGTT |
| | | CTGGGCCAGTCCAAACGCGTTGACTTCTGCGGCAAGGGCTACCACCTGATGTCCTTCCCCCAGTCCGCTCCCCACG |
| | | GAGTGGTGTTCCTGCACGTGACCTACGTGCCTGCCCAGGAGAAGAACTTCACCACCGCCCCCGCCATTTGCCACGA |
| | | CGGAAAGGCCCACTTCCCCCGCGAGGGAGTGTTCGTTTCTAACGGCACTCACTGGTTCGTGACCCAGAGAAACTTCT |
| | | ACGAGCCCCAGATTATTACCACCGACAATACCTTCGTTTCTGGCAACTGCGACGTGGTTATCGGCATCGTGAACAACA |
| | | CCGTGTACGACCCCCTGCAACCCGAGCTGGATTCTTTCAAGGAGGAGCTGGATAAGTACTTTAAAAACCATACCTCCC |
| | | CCGACGTGGACCTGGGCGACATCTCTGGCATCAACGCCTCCGTGGTGAATATCCAGAAGGAGATCGATCGCCTGAA |
| | | CGAGGTGGCCAAGAACTTAAACGAATCTCTGATCGACCTGCAGGAGCTGGGCAAGTACGAGCAGTATATTAAGTGGC |
| | | CCTGGTATATCTGGTTAGGCTTTATCGCCGGCCTGATCGCCATTGTTATGGTGACCATCATGCTGTGCTGCATGACCT |
| | | CCTGCTGTTCCTGTTTAAAAGGCTGCTGCTCCTGCGGATCCTGCTGTAAATTCGACGAAGACGACTCCGAGCCCGTG |
| | | CTGAAAGGAGTTAAGTTACACTATACCTAAATGAGACCATA |
| 265 | 2A5681 | ATAGGTCTCACACACTAGGGCGCGCCAGTCCTCAAGCTGGGCCTGACACAACTGTGTTCACTAGCAACTACTCCCAG |
| | | ACACCATGGTACTCCCGACTCCTGAGGTACTCCCTGCCGTTACTGCCCTGTGGGGCAAGGTGATACTCCCTGAAGTT |
| | | GGTGGTGAGGCCCTGGGCAGGCTACTCCCGGTCTACCCTTGGACCCAGAGGTCGACTCGGACCGGCCACCATGTTC |
| | | GTGTTCCTGGTGCTGCTGCCCCTGGTGTCCTCCCAGTGCGTCAACCTGACCACCCGGACCCAACTGCCCCCTGCCTA |
| | | TACCAATTCCTTCACCCGGGGCGTCTACTACCCCGACAAGGTGTTCCGGTCCTCTGTGCTCCACTCCACCCAGGACC |
| | | TCTTCCTCCCCTTTTTCTCCAACGTGACCTGGTTCCACGCCATCCACGTGTCTGGCACAAACGGAACCAAGCGCTTCG |
| | | ACAACCCTGTGCTCCCCTTCAACGACGGCGTGTACTTCGCCTCCACCGAGAAGTCCAACATCATCCGCGGCTGGATC |
| | | TTCGGCACCACCCTCGACTCTAAGACCCAGTCCCTGCTGATTGTGAATAACGCCACCAACGTGGTGATCAAGGTGTG |
| | | CGAATTTCAGTTCTGCAACGACCCCTTCCTGGGCGTTTACTACCATAAGAACAACAAGTCCTGGATGGAGTCCGAGTT |
| | | CCGGGTGTATTCCTCTGCCAATAACTGCACCTTTGAGTACGTGTCCCAGCCCTTCCTGATGGACCTGGAGGGCAAGC |
| | | AGGGCAATTTTAAAAACCTGCGCGAGTTCGTGTTCAAGAACATCGACGGCTACTTCAAGATCTATTCCAAGCACACCC |
| | | CTATCAACCTGGTGCGCGACCTGCCCCAGGGCTTTTCCGCTCTGGAGCCCCTGGTTGACCTGCCCATCGGCATCAAC |
| | | ATCACCCGCTTCCAGACTTTACTGGCCCTGCACCGCTCCTACCTGACCCCCGGCGATTCCTCTTCTGGCTGGACAGC |
| | | CGGAGCCGCTGCCTACTACGTGGGATACCTGCAGCCCCGCACCTTCCTGCTGAAGTACAACGAGAACGGAACCATTA |
| | | CAGACGCTGTCGACTGTGCTCTGGATCCCCTGTCCGAAACAAAGTGCACCCTGAAGTCCTTCACCGTGGAGAAGGGC |
| | | ATCTACCAGACCTCCAACTTCCGCGTGCAGCCCACCGAGTCCATCGTGAGATTCCCCAACATCACTAACCTGTGCCC |
| | | CTTCGGCGAGGTGTTCAACGCCACCCGCTTCGCCTCCGTGTACGCCTGGAACCGCAAGCGCATCTCCAACTGCGTT |
| | | GCCGACTACTCTGTGCTCTACAACTCCGCCTCCTTCTCCACCTTCAAGTGCTACGGCGTGTCCCCCACCAAGCTGAA |
| | | CGACCTGTGCTTCACCAACGTGTACGCCGACTCCTTCGTGATCCGCGGCGACGAGGTGCGCCAGATCGCCCCCGGC |
| | | CAGACCGGAAAGATCGCCGATTACAATTACAAGCTGCCCGACGACTTCACCGGCTGCGTGATCGCCTGGAACTCCAA |
| | | CAATCTGGACTCCAAGGTTGGCGGCAACTACAACTACCTGTACCGCCTGTTCCGCAAGTCCAACCTGAAGCCCTTCG |
| | | AGCGGGACATTTCCACAGAGATTTACCAGGCTGGCTCCACCCCCTGTAACGGAGTGGAGGGCTTCAACTGCTACTTC |
| | | CCCCTGCAGTCCTACGGCTTCCAGCCCACCAACGGCGTGGGCTACCAGCCCTACCGCGTGGTGGTGCTGTCCTTCG |
| | | AGCTGCTGCACGCCCCTGCCACCGTGTGCGGCCCCAAGAAGTCCACCAACCTGGTGAAGAACAAGTGCGTGAACTT |
| | | CAACTTCAACGGACTGACAGGCACCGGCGTGCTGACCGAGTCCAACAAGAAGTTCCTGCCCTTCCAGCAGTTCGGCC |
| | | GGGACATTGCCGACACCACCGACGCCGTGCGCGACCCCCAGACCCTGGAGATCCTGGACATCACCCCCTGCTCCTT |
| | | CGGCGGAGTGTCCGTGATCACCCCCGGCACCAACACCTCCAACCAGGTTGCCGTGCTGTACCAGGACGTTAACTGC |
| | | ACCGAGGTGCCCGTGGCCATCCACGCCGACCAGCTGACCCCCACCTGGCGCGTGTACTCCACCGGCTCCAACGTGT |
| | | TCCAGACCCGCGCCGGCTGCCTGATCGGCGCCGAGCACGTGAACAACTCCTACGAGTGCGACATCCCCATTGGCGC |
| | | CGGCATCTGCGCCTCCTACCAGACCCAGACCAACTCCCCTCGCCGCGCCCGGTCCGTCGCCTCCCAGTCCATCATC |
| | | GCCTACACCATGTCCCTGGGCGCCGAGAACTCCGTGGCCTACTCCAACAACTCCATTGCCATCCCCACCAACTTCAC |
| | | CATCTCCGTGACCACCGAAATCCTCCCCGTGTCCATGACCAAGACCTCCGTGGACTGCACCATGTACATCTGCGGCG |
| | | ACTCCACCGAGTGCTCCAACCTGCTGCTGCAGTACGGCTCCTTCTGCACCCAGCTGAATCGCGCCCTGACCGGCATC |
| | | GCCGTGGAGCAGGACAAGAACACCCAAGAGGTGTTCGCCCAGGTGAAGCAGATCTACAAGACACCTCCCATCAAGG |
| | | ACTTCGGCGGCTTCAACTTCTCCCAGATCCTGCCCGACCCCTCCAAGCCCTCCAAGCGCTCCTTCATCGAGGACCTG |
| | | CTGTTCAACAAGGTGACCCTGGCCGACGCCGGCTTCATCAAGCAGTACGGCGACTGTCTGGGAGACATTGCCGCCC |
| | | GCGACCTGATCTGCGCCCAGAAGTTCAACGGCTTAACCGTGCTGCCCCCTCTGCTGACCGACGAGATGATCGCCCA |
| | | GTACACATCCGCTCTGCTGGCCGGCACCATCACCTCCGGCTGGACCTTTGGCGCTGGCGCTGCCCTGCAGATCCCC |
| | | TTCGCTATGCAGATGGCCTACCGCTTCAACGGCATCGGCGTGACCCAGAACGTGCTGTACGAGAACCAGAAGCTGAT |
| | | CGCCAACCAGTTCAACTCCGCCATCGGCAAGATCCAGGACTCCCTGTCCTCCACCGCCTCCGCCCTGGGCAAGCTG |
| | | CAGGACGTGGTGAACCAGAACGCCCAGGCCCTGAACACCCTGGTGAAGCAGCTGTCCTCTAACTTCGGCGCCATCT |
| | | CCTCCGTGCTGAACGACATTCTGTCCCGCCTGGATCCTCCTGAGGCCGAGGTGCAAATCGATCGCCTCATCACCGGC |
| | | CGCCTGCAGTCCCTGCAGACCTACGTGACACAGCAGCTGATCCGCGCCGCCGAGATCCGCGCCTCCGCCAACCTGG |
| | | CCGCCACCAAGATGTCTGAGTGCGTGCTGGGCCAGTCCAAACGCGTTGACTTCTGCGGCAAGGGCTACCACCTGAT |
| | | GTCCTTCCCCCAGTCCGCCCCCCACGGCGTGGTGTTCCTGCACGTGACCTACGTGCCCGCCCAGGAGAAGAACTTC |
| | | ACTACCGCCCCCGCCATCTGCCACGACGGCAAGGCCCACTTCCCCCGCGAGGGCGTGTTCGTGTCCAACGGCACCC |
| | | ACTGGTTCGTGACCCAGCGCAACTTCTACGAGCCCCAGATCATCACCACCGACAACACCTTCGTGTCCGGCAACTGC |
| | | GACGTGGTGATTGGCATCGTGAACAACACCGTGTACGACCCCCTGCAGCCCGAGCTGGACTCCTTCAAGGAGGAGC |
| | | TGGACAAGTACTTTAAGAACCACACCTCCCCCGACGTGGACCTGGGCGACATCTCCGGCATCAACGCCTCCGTGGTG |
| | | AACATCCAGAAGGAGATCGACCGCCTGAACGAGGTGGCCAAGAACCTGAACGAGTCCCTGATCGACCTGCAGGAGC |
| | | TGGGCAAGTACGAACAGTACATCAAGTGGCCCTGGTACATCTGGCTGGGCTTCATCGCCGGCCTGATCGCCATCGTG |
| | | ATGGTGACCATCATGCTGTGCTGCATGACCTCTTGCTGTTCCTGTCTGAAGGGCTGCTGCTCCTGCGGCTCCTGCTG |
| | | CAAGTTCGACGAGGACGACTCCGAGCCCGTGCTGAAGGGCGTGAAGCTGCACTACACCTAAATGAGACCATA |
| 266 | 2A6681 | ATAGGTCTCACACACTAGGGCGCGCCAGTCCTCAAGCTGGGCCTGACACAACTGTGTTCACTAGCAACTACTCCCAG |
| | | ACACCATGGTACTCCCGACTCCTGAGGTACTCCCTGCCGTTACTGCCCTGTGGGGCAAGGTGATACTCCCTGAAGTT |
| | | GGTGGTGAGGCCCTGGGCAGGCTACTCCCGGTCTACCCTTGGACCCAGAGGTCGACTCGGACCGGCCACCATGTTC |
| | | GTGTTCCTGGTGCTGCTCCCCCTGGTGAGCTCCCAGTGCGTCAACCTGACCACACGGACCCAGCTGCCCCCCGCCT |
| | | ATACCAACAGCTTCACCCGCGGCGTCTACTACCCCGACAAGGTGTTCCGGAGCAGCGTGCTGCACAGCACCCAGGA |
| | | CCTCTTCCTCCCCTTTTTTAGCAACGTGACCTGGTTCCACGCCATCCACGTGAGTGGCACCAACGGCACCAAGCGGT |
| | | TCGACAACCCTGTGCTCCCCTTCAACGACGGCGTGTACTTCGCTAGCACAGAAAAAAGTAACATCATTCGGGGCTGG |
| | | ATCTTCGGCACCACACTGGATAGCAAAACCCAGAGCCTGCTGATTGTGAATAACGCCACCAACGTGGTGATCAAGGT |
| | | GTGCGAGTTCCAGTTCTGCAACGACCCCTTCCTGGGCGTGTACTACCACAAGAACAACAAGAGCTGGATGGAGAGCG |
| | | AGTTCCGCGTGTATAGCTCTGCCAACAACTGCACCTTCGAGTACGTGAGCCAGCCCTTCCTGATGGACCTGGAGGGC |
| | | AAGCAGGGCAATTTCAAAAACCTGCGGGAGTTCGTGTTCAAGAACATCGACGGCTACTTCAAGATCTACAGCAAGCAC |
| | | ACCCCCATCAACCTGGTGCGGGACCTGCCCCAGGGCTTTAGCGCTCTGGAGCCCCTGGTGGACCTGCCCATCGGCA |
| | | TCAACATCACCCGCTTCCAAACCCTGTTGGCCCTGCACCGGAGCTACCTGACCCCTGGCGATAGCAGTAGTGGCTGG |
| | | ACAGCCGGAGCCGCCGCCTACTACGTGGGATACCTGCAGCCCCGGACCTTCCTGCTGAAGTACAACGAGAACGGCA |
| | | CAATCACAGACGCCGTGGACTGCGCCCTGGACCCCCTGAGCGAAACAAAGTGCACCCTCAAAAGTTTCACCGTGGA |
| | | GAAGGGCATCTACCAGACCAGCAACTTCCGGGTGCAGCCCACCGAGAGCATCGTGCGGTTCCCCAACATCACCAAC |
| | | CTGTGCCCCTTCGGCGAGGTGTTCAACGCCACCCGGTTCGCCAGCGTGTACGCCTGGAACCGGAAGCGGATCAGCA |
| | | ACTGCGTGGCCGACTACAGCGTGCTGTATAACAGTGCCAGCTTCAGCACCTTCAAGTGCTACGGCGTGAGCCCCACC |
| | | AAGCTGAACGACCTGTGCTTCACCAACGTGTACGCCGACAGCTTCGTGATCCGGGGCGACGAGGTGCGGCAGATCG |
| | | CCCCCGGCCAGACCGGCAAGATCGCCGATTACAATTACAAGCTGCCCGACGATTTCACCGGCTGCGTGATCGCCTG |
| | | GAACAGCAACAACCTGGACAGCAAGGTGGGGGGCAACTACAACTACCTGTACCGGCTGTTCCGCAAAAGTAACCTGA |
| | | AGCCCTTCGAGCGGGACATCAGCACCGAGATCTACCAGGCCGGCAGTACCCCTTGCAACGGCGTCGAGGGCTTCAA |
| | | CTGCTACTTCCCCCTGCAGAGCTACGGCTTCCAGCCCACCAACGGCGTGGGCTACCAGCCCTACCGGGTGGTGGTT |
| | | CTGAGCTTCGAGCTGCTGCACGCCCCTGCCACCGTGTGCGGCCCCAAGAAAAGCACCAACCTGGTGAAGAACAAGT |
| | | GCGTGAATTTCAATTTTAACGGACTCACCGGAACCGGCGTGCTGACCGAGAGCAACAAGAAGTTCCTGCCCTTCCAG |
| | | CAATTCGGCCGGGATATTGCCGACACCACTGACGCCGTGCGGGACCCCCAGACCCTGGAGATCCTGGACATCACAC |
| | | CCTGTAGCTTCGGCGGGGTGAGCGTGATCACCCCCGGCACCAACACCAGCAACCAGGTGGCCGTGCTGTACCAGGA |
| | | CGTGAACTGCACCGAGGTGCCCGTGGCCATCCACGCCGACCAGCTGACCCCCACCTGGCGGGTGTACAGCACCGG |
| | | CAGCAACGTGTTCCAGACCCGGGCCGGCTGCCTGATCGGCGCCGAGCACGTGAATAACAGCTACGAGTGCGACATC |
| | | CCCATCGGAGCCGGAATCTGCGCCAGCTACCAGACCCAGACCAACAGCCCCCGGAGAGCCCGGAGCGTGGCCAGC |
| | | CAGAGCATTATTGCCTACACCATGAGCCTGGGCGCCGAGAACAGCGTGGCCTACAGCAACAATAGTATTGCCATCCC |
| | | CACCAACTTCACCATCAGCGTGACCACCGAAATCCTCCCCGTGAGCATGACCAAGACCAGCGTGGACTGCACCATGT |
| | | ACATCTGCGGCGACAGCACCGAGTGCAGCAACCTGCTCCTGCAGTACGGCAGCTTCTGCACCCAGCTGAACCGGGC |
| | | CCTGACCGGAATTGCCGTGGAGCAGGACAAGAACACCCAGGAGGTGTTCGCCCAGGTGAAGCAGATCTACAAGACA |
| | | CCTCCCATCAAGGACTTCGGCGGCTTCAACTTCAGCCAGATCCTGCCCGACCCCAGCAAGCCCAGCAAGCGGAGCT |
| | | TCATCGAGGACCTGTTGTTCAACAAGGTGACCCTGGCCGACGCCGGCTTCATCAAGCAGTACGGCGACTGTCTGGGA |
| | | GATATTGCCGCACGGGACCTGATCTGCGCCCAGAAGTTCAACGGCCTGACCGTGCTGCCCCCACTGCTGACCGACG |
| | | AGATGATCGCCCAGTACACAAGCGCTCTGCTCGCCGGCACCATCACCAGCGGCTGGACCTTCGGCGCCGGCGCTGC |
| | | CCTGCAGATCCCCTTCGCCATGCAGATGGCCTACCGGTTCAACGGCATCGGCGTGACCCAGAACGTGCTGTACGAG |
| | | AACCAGAAGCTGATCGCCAACCAGTTCAACAGCGCCATCGGCAAGATCCAGGACAGCCTGAGCTCCACCGCCAGCG |
| | | CCCTGGGCAAGCTGCAGGACGTGGTGAACCAGAACGCCCAGGCCCTGAACACCCTGGTGAAGCAGCTGAGCAGCAA |
| | | CTTCGGCGCCATCAGCTCTGTGCTGAACGACATCCTGAGCCGGCTGGATCCTCCTGAGGCCGAGGTGCAAATCGAT |
| | | CGGCTCATCACCGGCCGGCTGCAGAGCCTGCAGACCTACGTGACACAGCAGCTGATCCGGGCCGCCGAGATCCGG |
| | | GCCAGCGCCAACCTGGCCGCTACCAAGATGAGCGAGTGCGTGCTGGGCCAGAGCAAGCGGGTGGACTTCTGCGGC |
| | | AAGGGCTACCACCTGATGAGCTTCCCCCAGAGCGCCCCCCACGGCGTGGTGTTCCTGCACGTGACCTACGTGCCCG |
| | | CCCAGGAGAAGAACTTCACCACCGCCCCCGCCATCTGCCACGACGGCAAGGCCCACTTCCCCCGGGAGGGCGTGTT |
| | | CGTGAGCAACGGCACCCACTGGTTCGTGACCCAGCGGAACTTCTACGAGCCCCAGATCATAACCACAGACAACACCT |
| | | TCGTGAGCGGCAACTGCGACGTGGTCATCGGCATCGTGAACAACACCGTGTACGACCCCCTGCAGCCCGAGCTGGA |
| | | CAGCTTCAAGGAGGAACTGGACAAGTACTTCAAGAACCACACCAGCCCCGACGTGGACCTGGGCGACATCAGCGGC |
| | | ATCAACGCCAGCGTGGTGAACATCCAGAAGGAGATCGACCGGCTGAACGAGGTGGCCAAGAACCTGAACGAGAGCC |
| | | TGATCGACCTGCAGGAGCTGGGCAAGTACGAGCAGTACATCAAGTGGCCCTGGTACATCTGGCTGGGCTTTATTGCC |
| | | GGCCTGATCGCCATCGTGATGGTGACCATCATGCTGTGCTGTATGACAAGCTGTTGCAGTTGCCTGAAGGGCTGCTG |
| | | CAGCTGCGGCAGCTGCTGCAAGTTCGACGAGGACGACAGCGAGCCCGTGCTGAAGGGCGTGAAGCTGCACTACAC |
| | | CTAAATGAGACCATA |
| 267 | P51 | ATGTACCGGATGCAGTTGTTGTCCTGTATAGCTCTTTCCCTTGCATTGGTCACTAATTCTGTGAACCTCACCACTCGCA |
| | | CACAGCTACCTCCCGCGTACACTAATTCATTTACCAGGGGCGTCTATTATCCTGATAAGGTGTTCCGGAGTTCAGTGT |
| | | TGCATAGCACTCAAGACCTGTTCCTGCCCTTCTTCTCCAATGTCACTTGGTTTCATGCGATACATGTGTCTGGTACCAA |
| | | CGGAACGAAGAGATTTGATAACCCCGTACTGCCATTCAATGATGGCGTATACTTTGCTTCGACTGAAAAATCCAACAT |
| | | CATCAGGGGCTGGATTTTTGGTACAACGCTTGATTCCAAGACCCAGTCCCTCCTTATTGTGAACAATGCGACCAACGT |
| | | CGTGATAAAGGTCTGTGAGTTTCAGTTCTGCAATGACCCATTCCTTGGAGTGTATTATCACAAGAACAACAAATCGTGG |
| | | ATGGAGTCAGAGTTTAGGGTGTACAGCAGCGCTAACAACTGTACATTTGAGTATGTGAGTCAGCCGTTTCTGATGGAT |
| | | CTTGAGGGCAAACAGGGCAATTTTAAGAATCTCAGAGAATTTGTGTTCAAGAACATTGATGGTTACTTTAAGATCTATA |
| | | GCAAACATACGCCAATCAACTTGGTTCGTGATCTGCCACAGGGATTTAGCGCACTGGAACCTCTCGTTGACTTGCCCA |
| | | TAGGTATTAACATCACCAGGTTCCAGACGCTCTTGGCATTACACCGTAGTTATCTGACCCCCGGGGACTCCAGTTCCG |
| | | GATGGACTGCAGGAGCCGCTGCCTACTATGTGGGTTACCTCCAGCCCAGGACCTTTCTTTTGAAATATAACGAGAAC |
| | | GGCACAATCACTGATGCTGTGGACTGCGCATTGGATCCTTTGTCAGAGACTAAGTGCACTCTGAAGTCATTCACAGTC |
| | | GAGAAAGGCATTTACCAGACGTCTAACTTCAGGGTTCAGCCTACTGAGTCCATCGTGAGATTCCCAAACATCACAAAT |
| | | CTTTGTCCCTTCGGTGAGGTATTCAATGCGACACGATTTGCCTCAGTGTACGCGTGGAATCGGAAGAGGATCTCCAAT |
| | | TGCGTGGCCGACTACTCCGTCTTATACAACTCAGCTAGCTTTTCAACATTCAAGTGCTATGGCGTGAGCCCTACCAAG |
| | | CTCAATGACCTGTGCTTCACTAATGTGTATGCCGACTCTTTTGTCATTCGCGGCGACGAGGTCCGACAAATCGCACCG |
| | | GGCCAAACCGGTAAAATTGCCGACTACAACTACAAGCTGCCTGACGACTTCACCGGCTGCGTAATCGCCTGGAACAG |
| | | CAATAACCTGGATAGCAAAGTGGGCGGAAACTACAACTACCTGTACCGGCTCTTTAGAAAGTCCAACCTGAAACCATT |
| | | CGAGCGCGATATCTCGACCGAAATCTACCAGGCGGGCAGCACCCCCTGTAATGGTGTAGAAGGGTTCAATTGTTACT |
| | | TTCCACTCCAGAGTTATGGGTTCCAGCCGACCAATGGCGTCGGTTATCAACCATATCGCGTTGTGGTGTTGTCCTTTG |
| | | AGCTGCTACACGCCCCAGCTACAGTGTGCGGGCCAAAGAAAAGCACAAATCTTGTCAAGAACAAATGCGTTAACTTTA |
| | | ATTTTAATGGACTCACAGGTACAGGAGTCCTGACCGAATCTAATAAGAAGTTCCTGCCCTTTCAACAGTTCGGACGAG |
| | | ACATTGCCGACACCACCGATGCCGTTCGGGACCCACAGACCTTAGAAATTCTGGATATTACTCCATGTAGTTTTGGGG |
| | | GAGTGTCTGTCATCACCCCTGGCACTAATACATCTAACCAGGTTGCAGTCCTCTACCAGGATGTGAACTGTACCGAAG |
| | | TGCCGGTCGCTATTCACGCAGACCAGCTCACTCCTACCTGGCGGGTGTACTCCACAGGCTCTAACGTGTTTCAGACA |
| | | CGTGCAGGGTGCCTAATCGGCGCAGAGCATGTAAATAACTCCTATGAGTGTGATATCCCCATCGGAGCCGGGATCTG |
| | | CGCTTCCTACCAGACACAAACGAATAGTCCCGGATCTGCCTCAAGCGTGGCATCTCAATCCATTATAGCATATACGAT |
| | | GTCCCTTGGAGCTGAAAACAGCGTTGCGTATTCAAACAATAGTATCGCTATTCCAACCAATTTTACAATTAGCGTGACC |
| | | ACAGAAATACTCCCTGTGAGCATGACCAAGACCAGTGTAGACTGTACTATGTACATCTGCGGCGACAGTACTGAGTGT |
| | | AGCAATCTGCTGCTACAGTATGGGTCCTTCTGTACTCAGCTTAATCGGGCTCTCACCGGAATCGCTGTAGAGCAAGAT |
| | | AAAAACACACAAGAAGTGTTTGCTCAAGTGAAGCAGATCTATAAGACACCTCCCATCAAGGATTTCGGTGGGTTCAAC |
| | | TTTAGCCAGATTCTGCCCGATCCGTCTAAACCGTCCAAGCGAAGTTTCATCGAAGACCTGCTTTTCAATAAGGTCACG |
| | | CTGGCAGATGCTGGATTTATCAAACAGTACGGCGACTGTCTGGGCGATATCGCCGCAAGAGACTTGATATGCGCCCA |
| | | AAAGTTTAATGGGTTAACCGTCCTTCCACCGCTCCTGACAGACGAGATGATCGCCCAGTATACAAGTGCCTTATTAGC |
| | | TGGGACCATTACTAGTGGATGGACATTTGGCGCCGGGGCTGCTCTACAGATACCCTTCGCCATGCAGATGGCTTACC |
| | | GCTTCAACGGAATCGGAGTTACCCAGAACGTACTGTACGAAAATCAGAAACTCATAGCTAATCAATTTAACTCTGCCAT |
| | | CGGGAAGATTCAGGATTCCCTGTCGTCTACAGCGTCCGCCTTGGGGAAACTGCAAGATGTAGTGAACCAGAACGCCC |
| | | AGGCCTTAAATACTCTGGTCAAGCAGTTATCTTCAAATTTCGGAGCAATTAGCTCTGTGTTGAACGATATTCTTTCCAG |
| | | GCTGGACCCTCCAGAAGCCGAAGTGCAAATAGACCGGCTCATCACGGGGCGCTTGCAAAGCCTGCAAACCTATGTCA |
| | | CCCAGCAACTGATTCGAGCAGCCGAGATCCGGGCCAGTGCTAATCTGGCCGCCACAAAAATGAGCGAGTGCGTCCT |
| | | CGGGCAGAGCAAACGCGTAGACTTCTGCGGTAAAGGCTATCACCTGATGAGCTTCCCTCAGAGCGCACCCCACGGG |
| | | GTGGTCTTCCTCCACGTTACCTACGTCCCTGCGCAGGAGAAGAACTTCACTACGGCCCCTGCAATTTGCCACGATGG |
| | | CAAGGCCCACTTTCCCAGGGAGGGGGTCTTCGTTTCCAACGGGACTCATTGGTTCGTGACTCAGAGAAATTTTTATGA |
| | | ACCTCAGATCATTACCACTGATAATACATTCGTGTCTGGCAACTGTGATGTGGTTATTGGGATAGTTAATAATACGGTA |
| | | TACGACCCACTCCAGCCCGAGCTGGACTCCTTCAAAGAGGAGCTGGACAAGTACTTTAAAAATCACACCTCACCTGAT |
| | | GTGGACCTAGGTGACATATCTGGCATAAATGCTAGCGTGGTTAACATTCAGAAGGAAATCGACAGACTCAACGAGGT |
| | | GGCCAAAAATCTGAACGAGAGTCTGATCGACCTGCAGGAGTTGGGAAAATATGAACAGGGGTATATCCCTGAAGCCC |
| | | CCAGGGACGGCCAGGCTTACGTCAGAAAGGATGGAGAGTGGGTGCTCTTGAGCACCTTCCTGTAA |
| 268 | P53 | ATGGGAGTCAAAGTTCTGTTTGCCCTGATCTGCATCGCTGTGGCCGAGGCCAAGgttaatcttacaaccagaactcaattaccccctgc |
| | | atacactaattctttcacacgtggtgtttattaccctgacaaagttttcagatcctcagttttacattcaactcaggacttgttcttacctttcttttccaatgttacttggttccatgcta |
| | | tacatgtctctgggaccaatggtactaagaggtttgataaccctgtcctaccatttaatgatggtgtttattttgcttccactgagaagtctaacataataagaggctggatttttggtact |
| | | actttagattcgaagacccagtccctacttattgttaataacgctactaatgttgttattaaagtctgtgaatttcaattttgtaatgatccatttttgggtgtttattaccacaaaaacaa |
| | | caaaagttggatggaaagtgagttcagagtttattctagtgcgaataattgcacttttgaatatgtctctcagccttttcttatggaccttgaaggaaaacagggtaatttcaaaaatctta |
| | | gggaatttgtgtttaagaatattgatggttattttaaaatatattctaagcacacgcctattaatttagtgcgtgatctccctcagggtttttcggctttagaaccattggtagatttgcca |
| | | ataggtattaacatcactaggtttcaaactttacttgctttacatagaagttatttgactcctggtgattcttcttcaggttggacagctggtgctgcagcttattatgtgggttatcttca |
| | | acctaggacttttctattaaaatataatgaaaatggaaccattacagatgctgtagactgtgcacttgaccctctctcagaaacaaagtgtacgttgaaatccttcactgtagaaaa |
| | | aggaatctatcaaacttctaactttagagtccaaccaacagaatctattgttagatttcctaatattacaaacttgtgcccttttggtgaagtttttaacgccaccagatttgcatccgt |
| | | gtatgcttggaacaggaagagaatcagcaactgtgttgctgattattctgtcctatataattccgcatcattttccacttttaagtgttatggagtgtctcctactaaattaaatgatctct |
| | | gctttactaatgtctatgcagattcatttgtaattagaggtgatgaagtcagacaaatcgctccagggcaaactggaaagattgctgattataattataaattaccagatgattttac |
| | | aggctgcgttatagcttggaattctaacaatcttgattctaaggttggtggtaattataattacctgtatagattgtttaggaagtctaatctcaaaccttttgagagagatatttcaact |
| | | gaaatctatcaggccggtagcacaccttgtaatggtgttgaaggttttaattgttactttcctttacaatcatatggtttccaacccactaatggtgttggttaccaaccatacagagta |
| | | gtagtactttcttttgaacttctacatgcaccagcaactgtttgtggacctaaaaagtctactaatttggttaaaaacaaatgtgtcaatttcaacttcaatggtttaacaggcacagg |
| | | tgttcttactgagtctaacaaaaagtttctgcctttccaacaatttggcagagacattgctgacactactgatgctgtccgtgatccacagacacttgagattcttgacattacaccat |
| | | gttcttttggtggtgtcagtgttataacaccaggaacaaatacttctaaccaggttgctgttctttatcaggatgttaactgcacagaagtccctgttgctattcatgcagatcaactta |
| | | ctcctacttggcgtgtttattctacaggttctaatgtttttcaaacacgtgcaggctgtttaataggggctgaacatgtcaacaactcatatgagtgtgacatacccattggtgcaggt |
| | | atatgcgctagttatcagactcagactaattctcctggcagcgccagcagtgtagctagtcaatccatcattgcctacactatgtcacttggtgcagaaaattcagttgcttactcta |
| | | ataactctattgccatacccacaaattttactattagtgttaccacagaaattctaccagtgtctatgaccaagacatcagtagattgtacaatgtacatttgtggtgattcaactgaa |
| | | tgcagcaatcttttgttgcaatatggcagtttttgtacacaattaaaccgtgctttaactggaatagctgttgaacaagacaaaaacacccaagaagtttttgcacaagtcaaaca |
| | | aatttacaaaacaccaccaattaaagattttggtggttttaatttttcacaaatattaccagatccatcaaaaccaagcaagaggtcatttattgaagatctacttttcaacaaagtg |
| | | acacttgcagatgctggcttcatcaaacaatatggtgattgccttggtgatattgctgctagggacctcatttgtgcacaaaagtttaacggccttactgttttgccacctttgctcac |
| | | agatgaaatgattgctcaatacacttctgcactgttagcgggtacaatcacttctggttggacctttggtgcaggtgctgcattacaaataccatttgctatgcaaatggcttatagg |
| | | tttaatggtattggagttacacagaatgttctctatgagaaccaaaaattgattgccaaccaatttaatagtgctattggcaaaattcaagactcactttcttccacagcaagtgca |
| | | cttggaaaacttcaagatgtggtcaaccaaaatgcacaagctttaaacacgcttgttaaacaacttagctccaattttggtgcaatttcaagtgttttaaatgatatcctttcacgtctt |
| | | gaccctcccgaggctgaagtgcaaattgataggttgatcacaggcagacttcaaagtttgcagacatatgtgactcaacaattaattagagctgcagaaatcagagcttctgct |
| | | aatcttgctgctactaaaatgtcagagtgtgtacttggacaatcaaaaagagttgatttttgtggaaagggctatcatcttatgtccttccctcagtcagcacctcatggtgtagtcttc |
| | | ttgcatgtgacttatgtccctgcacaagaaaagaacttcacaactgctcctgccatttgtcatgatggaaaagcacactttcctcgtgaaggtgtctttgtttcaaatggcacacact |
| | | ggtttgtaacacaaaggaatttttatgaaccacaaatcattactacagacaacacatttgtgtctggtaactgtgatgttgtaataggaattgtcaacaacacagtttatgatcctttg |
| | | caacctgaattagactcattcaaggaggagttagataaatattttaagaatcatacatcaccagatgttgatttaggtgacatctctggcattaatgcttcagttgtaaacattcaaa |
| | | aagaaattgaccgcctcaatgaggttgccaagaatttaaatgaatctctcatcgatctccaagaacttggaaagtatgagcagGGGTATATCCCTGAAGCCCC |
| | | CAGGGACGGCCAGGCTTACGTCAGAAAGGATGGAGAGTGGGTGCTCTTGAGCACCTTCCTG |
| 269 | P55 | ATGGGAGTCAAAGTTCTGTTTGCCCTGATCTGCATCGCTGTGGCCGAGGCCAAGgttaatcttacaaccagaactcaattaccccctgc |
| | | atacactaattctttcacacgtggtgtttattaccctgacaaagttttcagatcctcagttttacattcaactcaggatttgttcttacctttcttttccaatgttacttggttccatgcta |
| | | tacatgtctctgggaccaatggtactaagaggtttgataaccctgtcctaccatttaatgatggtgtttattttgcttccactgagaagtctaacataataagaggctggatctttggtact |
| | | actttagattcgaagacccagtccctacttattgttaataacgctactaatgttgttattaaagtctgtgaatttcaattttgtaatgatccatttttgggtgtttattaccacaaaaacaa |
| | | caaaagttggatggaaagtgagttcagagtttattctagtgcgaataattgcacttttgaatatgtctctcagccttttcttatggaccttgaaggaaaacagggtaatttcaaaaatctta |
| | | gggaatttgtgtttaagaatattgatggttattttaaaatatattctaagcacacgcctattaatttagtgcgtgatctccctcagggtttttcggctttagaaccattggtagatttgcca |
| | | ataggtattaacatcactaggtttcaaactttacttgctttacatagaagttatttgactcctggtgattcttcttcaggttggacagctggtgctgcagcttattatgtgggttatcttca |
| | | acctaggacttttctattaaaatataatgaaaatggaaccattacagatgctgtagactgtgcacttgaccctctctcagaaacaaagtgtacgttgaaatccttcactgtagaaaa |
| | | aggaatctatcaaacttctaactttagagtccaaccaacagaatctattgttagatttcctaatattacaaacttgtgcccttttggtgaagtttttaacgccaccagatttgcatccgt |
| | | gtatgcttggaacaggaagagaatcagcaactgtgttgctgattattctgtcctatataattccgcatcattttccacttttaagtgttatggagtgtctcctactaaattaaatgatctct |
| | | gctttactaatgtctatgcagattcatttgtaattagaggtgatgaagtcagacaaatcgctccagggcaaactggaaagattgctgattataattataaattaccagatgattttac |
| | | aggctgcgttatagcttggaattctaacaatcttgattctaaggttggtggtaattataattacctgtatagattgtttaggaagtctaatctcaaaccttttgagagagatatttcaact |
| | | gaaatctatcaggccggtagcacaccttgtaatggtgttgaaggttttaattgttactttcctttacaatcatatggtttccaacccactaatggtgttggttaccaaccatacagagta |
| | | gtagtactttcttttgaacttctacatgcaccagcaactgtttgtggacctaaaaagtctactaatttggttaaaaacaaatgtgtcaatttcaacttcaatggtttaacaggcacagg |
| | | tgttcttactgagtctaacaaaaagtttctgcctttccaacaatttggcagagacattgctgacactactgatgctgtccgtgatccacagacacttgagattcttgacattacaccat |
| | | gttcttttggtggtgtcagtgttataacaccaggaacaaatacttctaaccaggttgctgttctttatcaggatgttaactgcacagaagtccctgttgctattcatgcagatcaactta |
| | | ctcctacttggcgtgtttattctacaggttctaatgtttttcaaacacgtgcaggctgtttaataggggctgaacatgtcaacaactcatatgagtgtgacatacccattggtgcaggt |
| | | atatgcgctagttatcagactcagactaattctcctggcagcgccagcagtgtagctagtcaatccatcattgcctacactatgtcacttggtgcagaaaattcagttgcttactcta |
| | | ataactctattgccatacccacaaattttactattagtgttaccacagaaattctaccagtgtctatgaccaagacatcagtagattgtacaatgtacatttgtggtgattcaactgaa |
| | | tgcagcaatcttttgttgcaatatggcagtttttgtacacaattaaaccgtgctttaactgggatagctgttgaacaagacaaaaacacccaagaagtttttgcacaagtcaaaca |
| | | aatttacaaaacaccaccaattaaagattttggtggttttaatttttcacaaatattaccagatccatcaaaaccaagcaagaggtcatttattgaagatctacttttcaacaaagtg |
| | | acacttgcagatgctggcttcatcaaacaatatggtgattgccttggtgatattgctgctagggacctcatttgtgcacaaaagtttaacggccttactgttttgccacctttgctcac |
| | | agatgaaatgattgctcaatacacttctgcactgttagcgggtacaatcacttctggttggacctttggtgcaggtgctgcattacaaataccatttgctatgcaaatggcttatagg |
| | | tttaatggtattggagttacacagaatgttctctatgagaaccaaaaattgattgccaaccaatttaatagtgccattggcaaaattcaagactcactttcttccacagcaagtgca |
| | | cttggaaaacttcaagatgtggtcaaccaaaatgcacaagctttaaacacgcttgttaaacaacttagctccaattttggtgcaatttcaagtgttttaaatgatatcctttcacgtctt |
| | | gaccctcccgaggctgaagtgcaaattgataggttgatcacaggcagacttcaaagtttgcagacatatgtgactcaacaattaattagagctgcagaaatcagagcttctgct |
| | | aatcttgctgctactaaaatgtcagagtgtgtacttggacaatcaaaaagagttgatttttgtggaaagggctatcatcttatgtccttccctcagtcagcacctcatggtgtagtcttc |
| | | ttgcatgtgacttatgtccctgcacaagaaaagaacttcacaactgctcctgccatttgtcatgatggaaaagcacactttcctcgtgaaggtgtctttgtttcaaatggcacacact |
| | | ggtttgtaacacaaaggaatttttatgaaccacaaatcattactacagacaacacatttgtgtctggtaactgtgatgttgtaataggaattgtcaacaacacagtttatgatcctttg |
| | | caacctgaattagactcattcaaggaggagttagataaatattttaagaatcatacatcaccagatgttgatttaggtgacatctctggcattaatgcttcagttgtaaacattcaaa |
| | | aagaaattgaccgcctcaatgaggttgccaagaatttaaatgaatctctcatcgatctccaagaacttggaaagtatgagcagtatataaaatggccatggtacatttggctag |
| | | gttttatagctggcttgattgccatagtaatggtgacaattatgctttgctgtatgaccagttgctgtagttgtctcaagggctgttgttcttgtggatcctgctgcaaatttgatgaagac |
| | | gactctgagccagtgctcaaaggagtcaaattacattacaca |
| 270 | P57 | ATGGGAGTCAAAGTTCTGTTTGCCCTGATCTGCATCGCTGTGGCCGAGGCCAAGagagtccaaccaacagaatctattgttagatttcc |
| | | taatattacaaacttgtgcccttttggtgaagtttttaacgccaccagatttgcatccgtgtatgcttggaacaggaagagaatcagcaactgtgttgctgattattctgtcctatataa |
| | | ttccgcatcattttccacttttaagtgttatggagtgtctcctactaaattaaatgatctctgctttactaatgtctatgcagattcatttgtaattagaggtgatgaagtcagacaaatcg |
| | | ctccagggcaaactggaaagattgctgattataattataaattaccagatgattttacaggctgcgttatagcttggaattctaacaatcttgattctaaggttggtggtaattataatt |
| | | acctgtatagattgtttaggaagtctaatctcaaaccttttgagagagatatttcaactgaaatctatcaggccggtagcacaccttgtaatggtgttgaaggttttaattgttactttcc |
| | | tttacaatcatatggtttccaacccactaatggtgttggttaccaaccatacagagtagtagtactttcttttgaacttctacatgcaccagcaactgtttgtggacctaaaaagtcta |
| | | ctaatttggttaaaaacaaatgtgtcaatttcGGGTATATCCCTGAAGCCCCCAGGGACGGCCAGGCTTACGTCAGAAAGGATGGAGA |
| | | GTGGGTGCTCTTGAGCACCTTCCTGTAA |
| 271 | P58 | atgtttgtttttcttgttttattgccactagtctctagtcagtgtgttaatcttacaaccagaactcaattaccccctgcatacactaattctttcacacgtggtgtttattaccctgacaa |
| | | agttttcagatcctcagttttacattcaactcaggatttgttcttacctttcttttccaatgttacttggttccatgctatacatgtctctgggaccaatggtactaagaggtttgataacc |
| | | ctgtcctaccatttaatgatggtgtttattttgcttccactgagaagtctaacataataagaggctggatctttggtactactttagattcgaagacccagtccctacttattgttaataac |
| | | gctactaatgttgttattaaagtctgtgaatttcaattttgtaatgatccatttttgggtgtttattaccacaaaaacaacaaaagttggatggaaagtgagttcagagtttattctagtgc |
| | | gaataattgcacttttgaatatgtctctcagccttttcttatggaccttgaaggaaaacagggtaatttcaaaaatcttagggaatttgtgtttaagaatattgatggttattttaaaatat |
| | | attctaagcacacgcctattaatttagtgcgtgatctccctcagggtttttcggctttagaaccattggtagatttgccaataggtattaacatcactaggtttcaaactttacttgcttta |
| | | catagaagttatttgactcctggtgattcttcttcaggttggacagctggtgctgcagcttattatgtgggttatcttcaacctaggacttttctattaaaatataatgaaaatggaaccat |
| | | tacagatgctgtagactgtgcacttgaccctctctcagaaacaaagtgtacgttgaaatccttcactgtagaaaaaggaatctatcaaacttctaactttagagtccaaccaacaga |
| | | atctattgttagatttcctaatattacaaacttgtgcccttttggtgaagtttttaacgccaccagatttgcatccgtgtatgcttggaacaggaagagaatcagcaactgtgttgctga |
| | | ttattctgtcctatataattccgcatcattttccacttttaagtgttatggagtgtctcctactaaattaaatgatctctgctttactaatgtctatgcagattcatttgtaattagaggtg |
| | | atgaagtcagacaaatcgctccagggcaaactggaaagattgctgattataattataaattaccagatgattttacaggctgcgttatagcttggaattctaacaatcttgattctaaggtt |
| | | ggtggtaattataattacctgtatagattgtttaggaagtctaatctcaaaccttttgagagagatatttcaactgaaatctatcaggccggtagcacaccttgtaatggtgttgaag |
| | | gttttaattgttactttcctttacaatcatatggtttccaacccactaatggtgttggttaccaaccatacagagtagtagtactttcttttgaacttctacatgcaccagcaactgtttgt |
| | | ggacctaaaaagtctactaatttggttaaaaacaaatgtgtcaatttcaacttcaatggtttaacaggcacaggtgttcttactgagtctaacaaaaagtttctgcctttccaacaattt |
| | | ggcagagacattgctgacactactgatgctgtccgtgatccacagacacttgagattcttgacattacaccatgttcttttggtggtgtcagtgttataacaccaggaacaaatact |
| | | tctaaccaggttgctgttctttatcagggtgttaactgcacagaagtccctgttgctattcatgcagatcaacttactcctacttggcgtgtttattctacaggttctaatgtttttcaaac |
| | | acgtgcaggctgtttaataggggctgaacatgtcaacaactcatatgagtgtgacatacccattggtgcaggtatatgcgctagttatcagactcagactaattctcctggcagcg |
| | | ccagcagtgtagctagtcaatccatcattgcctacactatgtcacttggtgcagaaaattcagttgcttactctaataactctattgccatacccacaaattttactattagtgttacca |
| | | cagaaattctaccagtgtctatgaccaagacatcagtagattgtacaatgtacatttgtggtgattcaactgaatgcagcaatcttttgttgcaatatggcagtttttgtacacaatta |
| | | aaccgtgctttaactgggatagctgttgaacaagacaaaaacacccaagaagtttttgcacaagtcaaacaaatttacaaaacaccaccaattaaagattttggtggttttaatt |
| | | tttcacaaatattaccagatccatcaaaaccaagcaagaggtcatttattgaagatctacttttcaacaaagtgacacttgcagatgctggcttcatcaaacaatatggtgattgc |
| | | cttggtgatattgctgctagggacctcatttgtgcacaaaagtttaacggccttactgttttgccacctttgctcacagatgaaatgattgctcaatacacttctgcactgttagcgggt |
| | | acaatcacttctggttggacctttggtgcaggtgctgcattacaaataccatttgctatgcaaatggcttataggtttaatggtattggagttacacagaatgttctctatgagaacca |
| | | aaaattgattgccaaccaatttaatagtgccattggcaaaattcaagactcactttcttccacagcaagtgcacttggaaaacttcaagatgtggtcaaccaaaatgcacaagc |
| | | tttaaacacgcttgttaaacaacttagctccaattttggtgcaatttcaagtgttttaaatgatatcctttcacgtcttgaccctcccgaggctgaagtgcaaattgataggttgatcac |
| | | aggcagacttcaaagtttgcagacatatgtgactcaacaattaattagagctgcagaaatcagagcttctgctaatcttgctgctactaaaatgtcagagtgtgtacttggacaat |
| | | caaaaagagttgatttttgtggaaagggctatcatcttatgtccttccctcagtcagcacctcatggtgtagtcttcttgcatgtgacttatgtccctgcacaagaaaagaacttcac |
| | | aactgctcctgccatttgtcatgatggaaaagcacactttcctcgtgaaggtgtctttgtttcaaatggcacacactggtttgtaacacaaaggaatttttatgaaccacaaatcatt |
| | | actacagacaacacatttgtgtctggtaactgtgatgttgtaataggaattgtcaacaacacagtttatgatcctttgcaacctgaattagactcattcaaggaggagttagataaa |
| | | tattttaagaatcatacatcaccagatgttgatttaggtgacatctctggcattaatgcttcagttgtaaacattcaaaaagaaattgaccgcctcaatgaggttgccaagaatttaa |
| | | atgaatctctcatcgatctccaagaacttggaaagtatgagcagtatataaaatggccatggtacatttggctaggttttatagctggcttgattgccatagtaatggtgacaattat |
| | | gctttgctgtatgaccagttgctgtagttgtctcaagggctgttgttcttgtggatcctgctgcaaatttgatgaagacgactctgagccagtgctcaaaggagtcaaattacattac |
| | | aca |
| 272 | 2A2361 | ATGGGAGTCAAAGTTCTGTTTGCCCTGATCTGCATTGCTGTGGCCGAGGCCAAGAGAGTCCAACCAACAGAATCTATT |
| | | GTTAGATTTCCTAATATTACAAACTTGTGCCCTTTTGGTGAAGTTTTTAACGCCACCAGATTTGCATCCGTGTATGCTTG |
| | | GAACAGGAAGAGAATCAGCAACTGTGTTGCTGATTATTCTGTCCTATATAATTCCGCATCATTTTCCACTTTTAAGTGTT |
| | | ATGGAGTGTCTCCTACTAAATTAAATGATCTCTGCTTTACTAATGTCTATGCAGATTCATTTGTAATTAGAGGTGATGAA |
| | | GTCAGACAAATCGCTCCAGGGCAAACTGGAAAGATTGCTGATTATAATTATAAATTACCAGATGATTTTACAGGCTGCG |
| | | TTATAGCTTGGAATTCTAACAATCTTGATTCTAAGGTTGGTGGTAATTATAATTACCTGTATAGATTGTTTAGGAAGTCT |
| | | AATCTCAAACCTTTTGAGAGAGATATTTCAACTGAAATCTATCAGGCCGGTAGCACACCTTGTAATGGTGTTGAAGGTT |
| | | TTAATTGTTACTTTCCTTTACAATCATATGGTTTCCAACCCACTAATGGTGTTGGTTACCAACCATACAGAGTAGTAGTA |
| | | CTTTCTTTTGAACTTCTACATGCACCAGCAACTGTTTGTGGACCTAAAAAGTCTACTAATTTGGTTAAAAACAAATGTGT |
| | | CAATTTCGGAGGAGGAGGAAGCGGATACATCCCCGAGGCCCCCCGGGACGGCCAGGCCTACGTGCGGAAGGACGG |
| | | CGAGTGGGTGCTGCTCAGCACCTTCCTG |
| 273 | 2A3361 | ATGGGAGTCAAAGTTCTGTTTGCCCTGATCTGCATTGCTGTGGCCGAGGCCAAGAGAGTCCAACCAACAGAATCTATT |
| | | GTTAGATTTCCTAATATTACAAACTTGTGCCCTTTTGGTGAAGTTTTTAACGCCACCAGATTTGCATCCGTGTATGCTTG |
| | | GAACAGGAAGAGAATCAGCAACTGTGTTGCTGATTATTCTGTCCTATATAATTCCGCATCATTTTCCACTTTTAAGTGTT |
| | | ATGGAGTGTCTCCTACTAAATTAAATGATCTCTGCTTTACTAATGTCTATGCAGATTCATTTGTAATTAGAGGTGATGAA |
| | | GTCAGACAAATCGCTCCAGGGCAAACTGGAAAGATTGCTGATTATAATTATAAATTACCAGATGATTTTACAGGCTGCG |
| | | TTATAGCTTGGAATTCTAACAATCTTGATTCTAAGGTTGGTGGTAATTATAATTACCTGTATAGATTGTTTAGGAAGTCT |
| | | AATCTCAAACCTTTTGAGAGAGATATTTCAACTGAAATCTATCAGGCCGGTAGCACACCTTGTAATGGTGTTGAAGGTT |
| | | TTAATTGTTACTTTCCTTTACAATCATATGGTTTCCAACCCACTAATGGTGTTGGTTACCAACCATACAGAGTAGTAGTA |
| | | CTTTCTTTTGAACTTCTACATGCACCAGCAACTGTTTGTGGACCTAAAAAGTCTACTAATTTGGTTAAAAACAAATGTGT |
| | | CAATTTCGGAGGAGGAGGAAGCGGATACATCCCCGAGGCCCCCCGGGACGGCCAGGCCTACGTGCGGAAGGACGG |
| | | CGAGTGGGTGCTGCTCAGCACCTTCCTGGGAGGAGGAGGAAGCAGAGTCCAACCAACAGAATCTATTGTTAGATTTC |
| | | CTAATATTACAAACTTGTGCCCTTTTGGTGAAGTTTTTAACGCCACCAGATTTGCATCCGTGTATGCTTGGAACAGGAA |
| | | GAGAATCAGCAACTGTGTTGCTGATTATTCTGTCCTATATAATTCCGCATCATTTTCCACTTTTAAGTGTTATGGAGTGT |
| | | CTCCTACTAAATTAAATGATCTCTGCTTTACTAATGTCTATGCAGATTCATTTGTAATTAGAGGTGATGAAGTCAGACAA |
| | | ATCGCTCCAGGGCAAACTGGAAAGATTGCTGATTATAATTATAAATTACCAGATGATTTTACAGGCTGCGTTATAGCTT |
| | | GGAATTCTAACAATCTTGATTCTAAGGTTGGTGGTAATTATAATTACCTGTATAGATTGTTTAGGAAGTCTAATCTCAAA |
| | | CCTTTTGAGAGAGATATTTCAACTGAAATCTATCAGGCCGGTAGCACACCTTGTAATGGTGTTGAAGGTTTTAATTGTT |
| | | ACTTTCCTTTACAATCATATGGTTTCCAACCCACTAATGGTGTTGGTTACCAACCATACAGAGTAGTAGTACTTTCTTTT |
| | | GAACTTCTACATGCACCAGCAACTGTTTGTGGACCTAAAAAGTCTACTAATTTGGTTAAAAACAAATGTGTCAATTTC |
| 274 | 2A6361 | ATGGGAGTCAAAGTTCTGTTTGCCCTGATCTGCATTGCTGTGGCCGAGGCCAAGAGAGTCCAACCAACAGAATCTATT |
| | | GTTAGATTTCCTAATATTACAAACTTGTGCCCTTTTGGTGAAGTTTTTAACGCCACCAGATTTGCATCCGTGTATGCTTG |
| | | GAACAGGAAGAGAATCAGCAACTGTGTTGCTGATTATTCTGTCCTATATAATTCCGCATCATTTTCCACTTTTAAGTGTT |
| | | ATGGAGTGTCTCCTACTAAATTAAATGATCTCTGCTTTACTAATGTCTATGCAGATTCATTTGTAATTAGAGGTGATGAA |
| | | GTCAGACAAATCGCTCCAGGGCAAACTGGAAAGATTGCTGATTATAATTATAAATTACCAGATGATTTTACAGGCTGCG |
| | | TTATAGCTTGGAATTCTAACAATCTTGATTCTAAGGTTGGTGGTAATTATAATTACCTGTATAGATTGTTTAGGAAGTCT |
| | | AATCTCAAACCTTTTGAGAGAGATATTTCAACTGAAATCTATCAGGCCGGTAGCACACCTTGTAATGGTGTTGAAGGTT |
| | | TTAATTGTTACTTTCCTTTACAATCATATGGTTTCCAACCCACTAATGGTGTTGGTTACCAACCATACAGAGTAGTAGTA |
| | | CTTTCTTTTGAACTTCTACATGCACCAGCAACTGTTTGTGGACCTAAAAAGTCTACTAATTTGGTTAAAAACAAATGTGT |
| | | CAATTTCGACATCATTAAGCTGCTCAACGAGCAGGTGAACAAGGAGATGAACAGCTCCAACCTGTACATGAGCATGAG |
| | | CAGCTGGTGCTACACCCACAGCCTGGACGGCGCCGGCCTGTTCCTGTTTGACCACGCCGCTGAGGAATACGAGCAC |
| | | GCCAAGAAACTGATCGTGTTCCTGAACGAGAACAACGTGCCCGTGCAGCTGACCAGCATCAGCGCCCCCGAGCACA |
| | | AGTTCGAGAGCCTGACCCAGATCTTCCAGAAGGCCTACGAGCACGAGCAGCACATCAGCGAGAGCATCAACAACATC |
| | | GTCGACCACGCCATCAAGGGCAAGGACCACGCCACCTTCAACTTCCTGCAGTGGTACGTGAGCGAGCAGCACGAGG |
| | | AGGAGGTGCTGTTCAAGGACATCCTGGACAAGATCGAGCTGATCGGCAACGAGAACCACGGCCTGTACCTGGCCGA |
| | | CCAGTACGTGAAGGGCATCGCCAAGAGCCGCAAAAGT |
| 275 | 2A7361 | ATGGGAGTCAAAGTTCTGTTTGCCCTGATCTGCATTGCTGTGGCCGAGGCCAAGAGAGTCCAACCAACAGAATCTATT |
| | | GTTAGATTTCCTAATATTACAAACTTGTGCCCTTTTGGTGAAGTTTTTAACGCCACCAGATTTGCATCCGTGTATGCTTG |
| | | GAACAGGAAGAGAATCAGCAACTGTGTTGCTGATTATTCTGTCCTATATAATTCCGCATCATTTTCCACTTTTAAGTGTT |
| | | ATGGAGTGTCTCCTACTAAATTAAATGATCTCTGCTTTACTAATGTCTATGCAGATTCATTTGTAATTAGAGGTGATGAA |
| | | GTCAGACAAATCGCTCCAGGGCAAACTGGAAAGATTGCTGATTATAATTATAAATTACCAGATGATTTTACAGGCTGCG |
| | | TTATAGCTTGGAATTCTAACAATCTTGATTCTAAGGTTGGTGGTAATTATAATTACCTGTATAGATTGTTTAGGAAGTCT |
| | | AATCTCAAACCTTTTGAGAGAGATATTTCAACTGAAATCTATCAGGCCGGTAGCACACCTTGTAATGGTGTTGAAGGTT |
| | | TTAATTGTTACTTTCCTTTACAATCATATGGTTTCCAACCCACTAATGGTGTTGGTTACCAACCATACAGAGTAGTAGTA |
| | | CTTTCTTTTGAACTTCTACATGCACCAGCAACTGTTTGTGGACCTAAAAAGTCTACTAATTTGGTTAAAAACAAATGTGT |
| | | CAATTTCGGAGGAGGAGGAAGCGACATCATTAAGCTGCTCAACGAGCAGGTGAACAAGGAGATGAACAGCTCCAACC |
| | | TGTACATGAGCATGAGCAGCTGGTGCTACACCCACAGCCTGGACGGCGCCGGCCTGTTCCTGTTTGACCACGCCGC |
| | | TGAGGAATACGAGCACGCCAAGAAACTGATCGTGTTCCTGAACGAGAACAACGTGCCCGTGCAGCTGACCAGCATCA |
| | | GCGCCCCCGAGCACAAGTTCGAGAGCCTGACCCAGATCTTCCAGAAGGCCTACGAGCACGAGCAGCACATCAGCGA |
| | | GAGCATCAACAACATCGTCGACCACGCCATCAAGGGCAAGGACCACGCCACCTTCAACTTCCTGCAGTGGTACGTGA |
| | | GCGAGCAGCACGAGGAGGAGGTGCTGTTCAAGGACATCCTGGACAAGATCGAGCTGATCGGCAACGAGAACCACGG |
| | | CCTGTACCTGGCCGACCAGTACGTGAAGGGCATCGCCAAGAGCCGCAAAAGT |
| 276 | 2A8361 | ATGGGAGTCAAAGTTCTGTTTGCCCTGATCTGCATTGCTGTGGCCGAGGCCAAGAGAGTCCAACCAACAGAATCTATT |
| | | GTTAGATTTCCTAATATTACAAACTTGTGCCCTTTTGGTGAAGTTTTTAACGCCACCAGATTTGCATCCGTGTATGCTTG |
| | | GAACAGGAAGAGAATCAGCAACTGTGTTGCTGATTATTCTGTCCTATATAATTCCGCATCATTTTCCACTTTTAAGTGTT |
| | | ATGGAGTGTCTCCTACTAAATTAAATGATCTCTGCTTTACTAATGTCTATGCAGATTCATTTGTAATTAGAGGTGATGAA |
| | | GTCAGACAAATCGCTCCAGGGCAAACTGGAAAGATTGCTGATTATAATTATAAATTACCAGATGATTTTACAGGCTGCG |
| | | TTATAGCTTGGAATTCTAACAATCTTGATTCTAAGGTTGGTGGTAATTATAATTACCTGTATAGATTGTTTAGGAAGTCT |
| | | AATCTCAAACCTTTTGAGAGAGATATTTCAACTGAAATCTATCAGGCCGGTAGCACACCTTGTAATGGTGTTGAAGGTT |
| | | TTAATTGTTACTTTCCTTTACAATCATATGGTTTCCAACCCACTAATGGTGTTGGTTACCAACCATACAGAGTAGTAGTA |
| | | CTTTCTTTTGAACTTCTACATGCACCAGCAACTGTTTGTGGACCTAAAAAGTCTACTAATTTGGTTAAAAACAAATGTGT |
| | | CAATTTCCCCGGCAGCGGCTACATCCCCGAGGCCCCCCGGGACGGCCAGGCCTACGTGCGGAAGGACGGCGAGTG |
| | | GGTGCTGCTCAGCACCTTCCTGAGCGGCCGGAGCGGCGGAGACATCATTAAGCTGCTGAACGAGCAGGTGAACAAG |
| | | GAGATGAACAGCTCCAACCTGTACATGAGCATGAGCAGCTGGTGCTACACCCACAGCCTGGACGGCGCCGGCCTGT |
| | | TCCTGTTTGACCACGCCGCTGAGGAATACGAGCACGCCAAGAAACTGATCGTGTTCCTGAACGAGAACAACGTGCCC |
| | | GTGCAGCTGACCAGCATCAGCGCCCCCGAGCACAAGTTCGAGAGCCTGACCCAGATCTTCCAGAAGGCCTACGAGC |
| | | ACGAGCAGCACATCAGCGAGAGCATCAACAACATCGTCGACCACGCCATCAAGGGCAAGGACCACGCCACCTTCAA |
| | | CTTCCTGCAGTGGTACGTGAGCGAGCAGCACGAGGAGGAGGTGCTGTTCAAGGACATCCTGGACAAGATCGAGCTG |
| | | ATCGGCAACGAGAACCACGGCCTGTACCTGGCCGACCAGTACGTGAAGGGCATCGCCAAGAGCCGCAAAAGT |
| 277 | 2A9361 | ATGGGAGTCAAAGTTCTGTTTGCCCTGATCTGCATTGCTGTGGCCGAGGCCAAGAGAGTCCAACCAACAGAATCTATT |
| | | GTTAGATTTCCTAATATTACAAACTTGTGCCCTTTTGGTGAAGTTTTTAACGCCACCAGATTTGCATCCGTGTATGCTTG |
| | | GAACAGGAAGAGAATCAGCAACTGTGTTGCTGATTATTCTGTCCTATATAATTCCGCATCATTTTCCACTTTTAAGTGTT |
| | | ATGGAGTGTCTCCTACTAAATTAAATGATCTCTGCTTTACTAATGTCTATGCAGATTCATTTGTAATTAGAGGTGATGAA |
| | | GTCAGACAAATCGCTCCAGGGCAAACTGGAAAGATTGCTGATTATAATTATAAATTACCAGATGATTTTACAGGCTGCG |
| | | TTATAGCTTGGAATTCTAACAATCTTGATTCTAAGGTTGGTGGTAATTATAATTACCTGTATAGATTGTTTAGGAAGTCT |
| | | AATCTCAAACCTTTTGAGAGAGATATTTCAACTGAAATCTATCAGGCCGGTAGCACACCTTGTAATGGTGTTGAAGGTT |
| | | TTAATTGTTACTTTCCTTTACAATCATATGGTTTCCAACCCACTAATGGTGTTGGTTACCAACCATACAGAGTAGTAGTA |
| | | CTTTCTTTTGAACTTCTACATGCACCAGCAACTGTTTGTGGACCTAAAAAGTCTACTAATTTGGTTAAAAACAAATGTGT |
| | | CAATTTCATCAACCACGTGGGCGGAACCGGCGGCGCCATCATGGCCCCCGTGGCCGTGACCCGGCAGCTGGTGGG |
| | | CAGC |
| 278 | 2A0461 | ATGGGAGTCAAAGTTCTGTTTGCCCTGATCTGCATTGCTGTGGCCGAGGCCAAGAGAGTCCAACCAACAGAATCTATT |
| | | GTTAGATTTCCTAATATTACAAACTTGTGCCCTTTTGGTGAAGTTTTTAACGCCACCAGATTTGCATCCGTGTATGCTTG |
| | | GAACAGGAAGAGAATCAGCAACTGTGTTGCTGATTATTCTGTCCTATATAATTCCGCATCATTTTCCACTTTTAAGTGTT |
| | | ATGGAGTGTCTCCTACTAAATTAAATGATCTCTGCTTTACTAATGTCTATGCAGATTCATTTGTAATTAGAGGTGATGAA |
| | | GTCAGACAAATCGCTCCAGGGCAAACTGGAAAGATTGCTGATTATAATTATAAATTACCAGATGATTTTACAGGCTGCG |
| | | TTATAGCTTGGAATTCTAACAATCTTGATTCTAAGGTTGGTGGTAATTATAATTACCTGTATAGATTGTTTAGGAAGTCT |
| | | AATCTCAAACCTTTTGAGAGAGATATTTCAACTGAAATCTATCAGGCCGGTAGCACACCTTGTAATGGTGTTGAAGGTT |
| | | TTAATTGTTACTTTCCTTTACAATCATATGGTTTCCAACCCACTAATGGTGTTGGTTACCAACCATACAGAGTAGTAGTA |
| | | CTTTCTTTTGAACTTCTACATGCACCAGCAACTGTTTGTGGACCTAAAAAGTCTACTAATTTGGTTAAAAACAAATGTGT |
| | | CAATTTCGGAGGAGGAGGAAGCATCAACCACGTGGGGGAACCGGCGGCGCCATCATGGCCCCCGTGGCCGTGAC |
| | | CCGGCAGCTGGTGGGCAGC |
| 279 | 2A1461 | ATGGGAGTCAAAGTTCTGTTTGCCCTGATCTGCATTGCTGTGGCCGAGGCCAAGATCAACCACGTGGGGGGAACCGG |
| | | CGGCGCCATCATGGCCCCCGTGGCCGTGACCCGGCAGCTGGTGGGCAGCAGAGTCCAACCAACAGAATCTATTGTT |
| | | AGATTTCCTAATATTACAAACTTGTGCCCTTTTGGTGAAGTTTTTAACGCCACCAGATTTGCATCCGTGTATGCTTGGA |
| | | ACAGGAAGAGAATCAGCAACTGTGTTGCTGATTATTCTGTCCTATATAATTCCGCATCATTTTCCACTTTTAAGTGTTAT |
| | | GGAGTGTCTCCTACTAAATTAAATGATCTCTGCTTTACTAATGTCTATGCAGATTCATTTGTAATTAGAGGTGATGAAGT |
| | | CAGACAAATCGCTCCAGGGCAAACTGGAAAGATTGCTGATTATAATTATAAATTACCAGATGATTTTACAGGCTGCGTT |
| | | ATAGCTTGGAATTCTAACAATCTTGATTCTAAGGTTGGTGGTAATTATAATTACCTGTATAGATTGTTTAGGAAGTCTAA |
| | | TCTCAAACCTTTTGAGAGAGATATTTCAACTGAAATCTATCAGGCCGGTAGCACACCTTGTAATGGTGTTGAAGGTTTT |
| | | AATTGTTACTTTCCTTTACAATCATATGGTTTCCAACCCACTAATGGTGTTGGTTACCAACCATACAGAGTAGTAGTACT |
| | | TTCTTTTGAACTTCTACATGCACCAGCAACTGTTTGTGGACCTAAAAAGTCTACTAATTTGGTTAAAAACAAATGTGTCA |
| | | ATTTC |
| 280 | 2A2461 | ATGGGAGTCAAAGTTCTGTTTGCCCTGATCTGCATTGCTGTGGCCGAGGCCAAGATCAACCACGTGGGGGGAACCGG |
| | | CGGCGCCATCATGGCCCCCGTGGCCGTGACCCGGCAGCTGGTGGGCAGCGGAGGAGGAGGAAGCAGAGTCCAAC |
| | | CAACAGAATCTATTGTTAGATTTCCTAATATTACAAACTTGTGCCCTTTTGGTGAAGTTTTTAACGCCACCAGATTTGCA |
| | | TCCGTGTATGCTTGGAACAGGAAGAGAATCAGCAACTGTGTTGCTGATTATTCTGTCCTATATAATTCCGCATCATTTT |
| | | CCACTTTTAAGTGTTATGGAGTGTCTCCTACTAAATTAAATGATCTCTGCTTTACTAATGTCTATGCAGATTCATTTGTA |
| | | ATTAGAGGTGATGAAGTCAGACAAATCGCTCCAGGGCAAACTGGAAAGATTGCTGATTATAATTATAAATTACCAGATG |
| | | ATTTTACAGGCTGCGTTATAGCTTGGAATTCTAACAATCTTGATTCTAAGGTTGGTGGTAATTATAATTACCTGTATAGA |
| | | TTGTTTAGGAAGTCTAATCTCAAACCTTTTGAGAGAGATATTTCAACTGAAATCTATCAGGCCGGTAGCACACCTTGTA |
| | | ATGGTGTTGAAGGTTTTAATTGTTACTTTCCTTTACAATCATATGGTTTCCAACCCACTAATGGTGTTGGTTACCAACCA |
| | | TACAGAGTAGTAGTACTTTCTTTTGAACTTCTACATGCACCAGCAACTGTTTGTGGACCTAAAAAGTCTACTAATTTGGT |
| | | TAAAAACAAATGTGTCAATTTC |
| 281 | 2A7520 | ATGGGAGTCAAAGTTCTGTTTGCCCTGATCTGCATTGCTGTGGCCGAGGCCAAGAGAGTCCAACCAACAGAATCTATT |
| | | GTTAGATTTCCTAATATTACAAACTTGTGCCCTTTTGGTGAAGTTTTTAACGCCACCAGATTTGCATCCGTGTATGCTTG |
| | | GAACAGGAAGAGAATCAGCAACTGTGTTGCTGATTATTCTGTCCTATATAATTCCGCATCATTTTCCACTTTTAAGTGTT |
| | | ATGGAGTGTCTCCTACTAAATTAAATGATCTCTGCTTTACTAATGTCTATGCAGATTCATTTGTAATTAGAGGTGATGAA |
| | | GTCAGACAAATCGCTCCAGGGCAAACTGGAAAGATTGCTGATTATAATTATAAATTACCAGATGATTTTACAGGCTGCG |
| | | TTATAGCTTGGAATTCTAACAATCTTGATTCTAAGGTTGGTGGTAATTATAATTACCTGTATAGATTGTTTAGGAAGTCT |
| | | AATCTCAAACCTTTTGAGAGAGATATTTCAACTGAAATCTATCAGGCCGGTAGCACACCTTGTAATGGTGTTGAAGGTT |
| | | TTAATTGTTACTTTCCTTTACAATCATATGGTTTCCAACCCACTAATGGTGTTGGTTACCAACCATACAGAGTAGTAGTA |
| | | CTTTCTTTTGAACTTCTACATGCACCAGCAACTGTTTGTGGACCTAAAAAGTCTACTAATTTGGTTAAAAACAAATGTGT |
| | | CAATTTCGGGTATATCCCTGAAGCCCCCAGGGACGGCCAGGCTTACGTCAGAAAGGATGGAGAGTGGGTGCTCTTGA |
| | | GCACCTTCCTGTAA |
| 283 | Omicron | MFVFLVLLPLVSSQCVNLITRTQSYTNSFTRGVYYPDKVFRSSVLHSTQDLFLPFFSNVTWFHAISGTNGIKRFDNPVLPFN |
| | spike | DGVYFASTEKSNIIRGWIFGTTLDSKTQSLLIVNNATNVVIKVCEFQFCNDPFLDVYYHKNNKSWMESEFRVYSSANNCTFE |
| | protein | YVSQPFLMDLEGKQGNFKNLREFVFKNIDGYFKIYSKHTPINLGRDLPQGFSALEPLVDLPIGINITRFQTLLALHRSYLTPGD |
| | | SSSGWTAGAAAYYVGYLQPRTFLLKYNENGTITDAVDCALDPLSETKCTLKSFTVEKGIYQTSNFRVQPTESIVRFPNITNLC |
| | | PFDEVFNATRFASVYAWNRKRISNCVADYSVLYNFAPFFAFKCYGVSPTKLNDLCFTNVYADSFVIRGNEVSQIAPGQTGNI |
| | | ADYNYKLPDDFTGCVIAWNSNKLDSKVGGNYNYRYRLFRKSNLKPFERDISTEIYQAGNKPCNGVAGVNCYFPLQSYGFR |
| | | PTYGVGHQPYRVVVLSFELLHAPATVCGPKKSTNLVKNKCVNFNFNGLTGTGVLTESNKKFLPFQQFGRDIADTTDAVRDP |
| | | QTLEILDITPCSFGGVSVITPGTNTSNQVAVLYQGVNCTEVPVAIHADQLTPTWRVYSTGSNVFQTRAGCLIGAEYVNNSYE |
| | | CDIPIGAGICASYQTQTKSHRRARSVASQSIIAYTMSLGAENSVAYSNNSIAIPTNFTISVTTEILPVSMTKTSVDCTMYICGDS |
| | | TECSNLLLQYGSFCTQLKRALTGIAVEQDKNTQEVFAQVKQIYKTPPIKYFGGFNFSQILPDPSKPSKRSFIEDLLFNKVTLAD |
| | | AGFIKQYGDCLGDIAARDLICAQKFNGLTVLPPLLTDEMIAQYTSALLAGTITSGWTFGAGAALQIPFAMQMAYRFNGIGVTQ |
| | | NVLYENQKLIANQFNSAIGKIQDSLSSTASALGKLQDVVNHNAQALNTLVKQLSSKFGAISSVLNDILSRLDKVEAEVQIDRLI |
| | | TGRLQSLQTYVTQQLIRAAEIRASANLAATKMSECVLGQSKRVDFCGKGYHLMSFPQSAPHGVVFLHVTYVPAQEKNFTTA |
| | | PAICHDGKAHFPREGVFVSNGTHWFVTQRNFYEPQIITTDNTFVSGNCDVVIGIVNNTVYDPLQPELDSFKEELDKYFKNHT |
| | | SPDVDLGDISGINASVVNIQKEIDRLNEVAKNLNESLIDLQELGKYEQYIKWPWYIWLGFIAGLIAIVMVTIMLCCMTSCCSCLK |
| | | GCCSCGSCCKFDEDDSEPVLKGVKLHYT |
| 284 | Omicron- | MGVFLVLLPLVSSQCVNLITRTQSYTNSFTRGVYYPDKVFRSSVLHSTQDLFLPFFSNVTWFHAISGTNGIKRFDNPVLPFN |
| | BA52P | DGVYFASTEKSNIIRGWIFGTTLDSKTQSLLIVNNATNVVIKVCEFQFCNDPFLDVYYHKNNKSWMESEFRVYSSANNCTFE |
| | | YVSQPFLMDLEGKQGNFKNLREFVFKNIDGYFKIYSKHTPINLGRDLPQGFSALEPLVDLPIGINITRFQTLLALHRSYLTPGD |
| | | SSSGWTAGAAAYYVGYLQPRTFLLKYNENGTITDAVDCALDPLSETKCTLKSFTVEKGIYQTSNFRVQPTESIVRFPNITNLC |
| | | PFDEVFNATRFASVYAWNRKRISNCVADYSVLYNFAPFFAFKCYGVSPTKLNDLCFTNVYADSFVIRGNEVSQIAPGQTGNI |
| | | ADYNYKLPDDFTGCVIAWNSNNLDSKVGGNYNYRYRLFRKSNLKPFERDISTEIYQAGNKPCNGVAGVNCYFPLQSYGFR |
| | | PTYGVGHQPYRVVVLSFELLHAPATVCGPKKSTNLVKNKCVNFNFNGLTGTGVLTESNKKFLPFQQFGRDIADTTDAVRDP |
| | | QTLEILDITPCSFGGVSVITPGTNTSNQVAVLYQGVNCTEVPVAIHADQLTPTWRVYSTGSNVFQTRAGCLIGAEYVNNSYE |
| | | CDIPIGAGICASYQTQTKSHRRARSVASQSIIAYTMSLGAENSVAYSNNSIAIPTNFTISVTTEILPVSMTKTSVDCTMYICGDS |
| | | TECSNLLLQYGSFCTQLKRALTGIAVEQDKNTQEVFAQVKQIYKTPPIKYFGGFNFSQILPDPSKPSKRSFIEDLLFNKVTLAD |
| | | AGFIKQYGDCLGDIAARDLICAQKFNGLTVLPPLLTDEMIAQYTSALLAGTITSGWTFGAGAALQIPFAMQMAYRFNGIGVTQ |
| | | NVLYENQKLIANQFNSAIGKIQDSLSSTASALGKLQDVVNHNAQALNTLVKQLSSKFGAISSVLNDILSRLDPPEAEVQIDRLI |
| | | TGRLQSLQTYVTQQLIRAAEIRASANLAATKMSECVLGQSKRVDFCGKGYHLMSFPQSAPHGVVFLHVTYVPAQEKNFTTA |
| | | PAICHDGKAHFPREGVFVSNGTHWFVTQRNFYEPQIITTDNTFVSGNCDVVIGIVNNTVYDPLQPELDSFKEELDKYFKNHT |
| | | SPDVDLGDISGINASVVNIQKEIDRLNEVAKNLNESLIDLQELGKYEQYIKWPWYIWLGFIAGLIAIVMVTIMLCCMTSCCSCLK |
| | | GCCSCGSCCKFDEDDSEPVLKGVKLHYT |
| 285 | Spike- | MFVFLVLLPLVSSQCVNLTTRTQLPPAYTNSFTRGVYYPDKVFRSSVLHSTQDLFLPFFSNVTWFHAIHVSGTNGTKRFDN |
| | HexaP | PVLPFNDGVYFASTEKSNIIRGWIFGTTLDSKTQSLLIVNNATNVVIKVCEFQFCNDPFLGVYYHKNNKSWMESEFRVYSSA |
| | | NNCTFEYVSQPFLMDLEGKQGNFKNLREFVFKNIDGYFKIYSKHTPINLVRDLPQGFSALEPLVDLPIGINITRFQTLLALHRS |
| | | YLTPGDSSSGWTAGAAAYYVGYLQPRTFLLKYNENGTITDAVDCALDPLSETKCTLKSFTVEKGIYQTSNFRVQPTESIVRF |
| | | PNITNLCPFGEVFNATRFASVYAWNRKRISNCVADYSVLYNSASFSTFKCYGVSPTKLNDLCFTNVYADSFVIRGDEVRQIA |
| | | PGQTGKIADYNYKLPDDFTGCVIAWNSNNLDSKVGGNYNYLYRLFRKSNLKPFERDISTEIYQAGSTPCNGVEGFNCYFPL |
| | | QSYGFQPTNGVGYQPYRVVVLSFELLHAPATVCGPKKSTNLVKNKCVNFNFNGLTGTGVLTESNKKFLPFQQFGRDIADTT |
| | | DAVRDPQTLEILDITPCSFGGVSVITPGTNTSNQVAVLYQDVNCTEVPVAIHADQLTPTWRVYSTGSNVFQTRAGCLIGAEH |
| | | VNNSYECDIPIGAGICASYQTQTNSPRRARSVASQSIIAYTMSLGAENSVAYSNNSIAIPTNFTISVTTEILPVSMTKTSVDCT |
| | | MYICGDSTECSNLLLQYGSFCTQLNRALTGIAVEQDKNTQEVFAQVKQIYKTPPIKDFGGFNFSQILPDPSKPSKRSPIEDLL |
| | | FNKVTLADAGFIKQYGDCLGDIAARDLICAQKFNGLTVLPPLLTDEMIAQYTSALLAGTITSGWTFGAGPALQIPFPMQMAYR |
| | | FNGIGVTQNVLYENQKLIANQFNSAIGKIQDSLSSTPSALGKLQDVVNQNAQALNTLVKQLSSNFGAISSVLNDILSRLDPPE |
| | | AEVQIDRLITGRLQSLQTYVTQQLIRAAEIRASANLAATKMSECVLGQSKRVDFCGKGYHLMSFPQSAPHGVVFLHVTYVPA |
| | | QEKNFTTAPAICHDGKAHFPREGVFVSNGTHWFVTQRNFYEPQIITTDNTFVSGNCDVVIGIVNNTVYDPLQPELDSFKEEL |
| | | DKYFKNHTSPDVDLGDISGINASVVNIQKEIDRLNEVAKNLNESLIDLQELGKYEQYIKWPWYIWLGFIAGLIAIVMVTIMLCC |
| | | MTSCCSCLKGCCSCGSCCKFDEDDSEPVLKGVKLHYT- |
| 286 | BA5-Spike- | MGVFLVLLPLVSSQCVNLITRTQSYTNSFTRGVYYPDKVFRSSVLHSTQDLFLPFFSNVTWFHAISGTNGIKRFDNPVLPFN |
| | HexaP | DGVYFASTEKSNIIRGWIFGTTLDSKTQSLLIVNNATNVVIKVCEFQFCNDPFLDVYYHKNNKSWMESEFRVYSSANNCTFE |
| | | YVSQPFLMDLEGKQGNFKNLREFVFKNIDGYFKIYSKHTPINLGRDLPQGFSALEPLVDLPIGINITRFQTLLALHRSYLTPGD |
| | | SSSGWTAGAAAYYVGYLQPRTFLLKYNENGTITDAVDCALDPLSETKCTLKSFTVEKGIYQTSNFRVQPTESIVRFPNITNLC |
| | | PFDEVFNATRFASVYAWNRKRISNCVADYSVLYNFAPFFAFKCYGVSPTKLNDLCFTNVYADSFVIRGNEVSQIAPGQTGNI |
| | | ADYNYKLPDDFTGCVIAWNSNNLDSKVGGNYNYRYRLFRKSNLKPFERDISTEIYQAGNKPCNGVAGVNCYFPLQSYGFR |
| | | PTYGVGHQPYRVVVLSFELLHAPATVCGPKKSTNLVKNKCVNFNFNGLTGTGVLTESNKKFLPFQQFGRDIADTTDAVRDP |
| | | QTLEILDITPCSFGGVSVITPGTNTSNQVAVLYQGVNCTEVPVAIHADQLTPTWRVYSTGSNVFQTRAGCLIGAEYVNNSYE |
| | | CDIPIGAGICASYQTQTKSHRRARSVASQSIIAYTMSLGAENSVAYSNNSIAIPTNFTISVTTEILPVSMTKTSVDCTMYICGDS |
| | | TECSNLLLQYGSFCTQLKRALTGIAVEQDKNTQEVFAQVKQIYKTPPIKYFGGFNFSQILPDPSKPSKRSPIEDLLFNKVTLA |
| | | DAGFIKQYGDCLGDIAARDLICAQKFNGLTVLPPLLTDEMIAQYTSALLAGTITSGWTFGAGPALQIPFPMQMAYRENGIGVT |
| | | QNVLYENQKLIANQFNSAIGKIQDSLSSTPSALGKLQDVVNHNAQALNTLVKQLSSKFGAISSVLNDILSRLDPPEAEVQIDRL |
| | | ITGRLQSLQTYVTQQLIRAAEIRASANLAATKMSECVLGQSKRVDFCGKGYHLMSFPQSAPHGVVFLHVTYVPAQEKNFTT |
| | | APAICHDGKAHFPREGVFVSNGTHWFVTQRNFYEPQIITTDNTFVSGNCDVVIGIVNNTVYDPLQPELDSFKEELDKYFKNH |
| | | TSPDVDLGDISGINASVVNIQKEIDRLNEVAKNLNESLIDLQELGKYEQYIKWPWYIWLGFIAGLIAIVMVTIMLCCMTSCCSCL |
| | | KGCCSCGSCCKFDEDDSEPVLKGVKLHYT- |
| 287 | Secretion | MGVTAPRTLILLLSGALALTETWAGSAVGACVLCNSQTSLRCGACIRRPFLCCKCCYDHVISTSHKLVLSVNPYVCNAPGC |
| | signal | DVTDVTQLYLGGMSYYCKSHKPPISFPLCANGQVFGLYKNTCVGSDNVTDFNAIATCDWTNAGDYILANTCTERLKLFAAE |
| | sequence | TLKATEETFKLSYGIATVREVLSDRELHLSWEVGKPRPPLNRNYVFTGYRVTKNSKVQIGEYTFEKGDYGDAVVYRGTTTY |
| | (under- | KLNVGDYFVLTSHTVMPLSAPTLVPQEHYVRITGLYPTLNISDEFSSNVANYQKVGMQKYSTLQGPPGTGKSHFAIGLALYY |
| | lined) | PSARIVYTACSHAAVDALCEKALKYLPIDKCSRIIPARARVECFDKFKVNSTLEQYVFCTVNALPETTADIVVFDEISMATNYD |
| | NSP13 | LSVVNARLRAKHYVYIGDPAQLPAPRTLLTKGTLEPEYFNSVCRLMKTIGPDMFLGTCRRCPAEIVDTVSALVYDNKLKAHK |
| | | DKSAQCFKMFYKGVITHDVSSAINRPQIGVVREFLTRNPAWRKAVFISPYNSQNAVASKILGLPTQTVDSSQGSEYDYVIFT |
| | | QTTETAHSCNVNRFNVAITRAKVGILCIMSDRDLYDKLQFTSLEIPRRNVATLQ |
| 288 | Secretion | MGVTAPRTLILLLSGALALTETWAGSSADAQSFLNRVCGVSAARLTPCGTGTSTDVVYRAFDIYNDKVAGFAKFLKTNCCR |
| | signal | FQEKDEDDNLIDSYFVVKRHTFSNYQHEETIYNLLKDCPAVAKHDFFKFRIDGDMVPHISRQRLTKYTMADLVYALRHFDEG |
| | sequence | NCDTLKEILVTYNCCDDDYFNKKDWYDFVENPDILRVYANLGERVRQALLKTVQFCDAMRNAGIVGVLTLDNQDLNGNWY |
| | (under- | DFGDFIQTTPGSGVPVVDSYYSLLMPILTLTRALTAESHVDTDLTKPYIKWDLLKYDFTEERLKLFDRYFKYWDQTYHPNCV |
| | lined) | NCLDDRCILHCANFNVLFSTVFPPTSFGPLVRKIFVDGVPFVVSTGYHFRELGVVHNQDVNLHSSRLSFKELLVYAADPAMH |
| | NSP12 | AASGNLLLDKRTTCFSVAALTNNVAFQTVKPGNFNKDFYDFAVSKGFFKEGSSVELKHFFFAQDGNAAISDYDYYRYNLPT |
| | | MCDIRQLLFVVEVVDKYFDCYDGGCINANQVIVNNLDKSAGFPFNKWGKARLYYDSMSYEDQDALFAYTKRNVIPTITQMN |
| | | LKYAISAKNRARTVAGVSICSTMTNRQFHQKLLKSIAATRGATVVIGTSKFYGGWHNMLKTVYSDVENPHLMGWDYPKCD |
| | | RAMPNMLRIMASLVLARKHTTCCSLSHRFYRLANECAQVLSEMVMCGGSLYVKPGGTSSGDATTAYANSVFNICQAVTAN |
| | | VNALLSTDGNKIADKYVRNLQHRLYECLYRNRDVDTDFVNEFYAYLRKHFSMMILSDDAVVCFNSTYASQGLVASIKNFKSV |
| | | LYYQNNVFMSEAKCWTETDLTKGPHEFCSQHTMLVKQGDDYVYLPYPDPSRILGAGCFVDDIVKTDGTLMIERFVSLAIDA |
| | | YPLTKHPNQEYADVFHLYLQYIRKLHDELTGHMLDMYSVMLTNDNTSRYWEPEFYEAMYTPHTVLQ |
| 289 | Secretion | MGVTAPRTLILLLSGALALTETWAGSSKMSDVKCTSVVLLSVLQQLRVESSSKLWAQCVQLHNDILLAKDTTEAFEKMVSLL |
| | signal | SVLLSMQGAVDINKLCEEMLDNRATLQ |
| | sequence | |
| | (under- | |
| | lined) | |
| | NSP7 | |
| 290 | Concatemer | MGAVGACVLCNSQTSLRCGACIRRPFLCCKCCYDHVISTSHKLVLSVNPYVCNAPGCDVTDVTQLYLGGMSYYCKSHKPP |
| | (NSP13-G4S- | ISFPLCANGQVFGLYKNTCVGSDNVTDFNAIATCDWTNAGDYILANTCTERLKLFAAETLKATEETFKLSYGIATVREVLSDR |
| | NSP12-G4S- | ELHLSWEVGKPRPPLNRNYVFTGYRVTKNSKVQIGEYTFEKGDYGDAVVYRGTTTYKLNVGDYFVLTSHTVMPLSAPTLVP |
| | NSP7) | QEHYVRITGLYPTLNISDEFSSNVANYQKVGMQKYSTLQGPPGTGKSHFAIGLALYYPSARIVYTACSHAAVDALCEKALKY |
| | | LPIDKCSRIIPARARVECFDKFKVNSTLEQYVFCTVNALPETTADIVVFDEISMATNYDLSVVNARLRAKHYVYIGDPAQLPAP |
| | | RTLLTKGTLEPEYFNSVCRLMKTIGPDMFLGTCRRCPAEIVDTVSALVYDNKLKAHKDKSAQCFKMFYKGVITHDVSSAINR |
| | | PQIGVVREFLTRNPAWRKAVFISPYNSQNAVASKILGLPTQTVDSSQGSEYDYVIFTQTTETAHSCNVNRFNVAITRAKVGIL |
| | | CIMSDRDLYDKLQFTSLEIPRRNVATLQGGGGSGGGGSSADAQSFLNRVCGVSAARLTPCGTGTSTDVVYRAFDIYNDKV |
| | | AGFAKFLKTNCCRFQEKDEDDNLIDSYFVVKRHTFSNYQHEETIYNLLKDCPAVAKHDFFKFRIDGDMVPHISRQRLTKYTM |
| | | ADLVYALRHFDEGNCDTLKEILVTYNCCDDDYFNKKDWYDFVENPDILRVYANLGERVRQALLKTVQFCDAMRNAGIVGVL |
| | | TLDNQDLNGNWYDFGDFIQTTPGSGVPVVDSYYSLLMPILTLTRALTAESHVDTDLTKPYIKWDLLKYDFTEERLKLFDRYF |
| | | KYWDQTYHPNCVNCLDDRCILHCANFNVLFSTVFPPTSFGPLVRKIFVDGVPFVVSTGYHFRELGVVHNQDVNLHSSRLSF |
| | | KELLVYAADPAMHAASGNLLLDKRTTCFSVAALTNNVAFQTVKPGNFNKDFYDFAVSKGFFKEGSSVELKHFFFAQDGNAA |
| | | ISDYDYYRYNLPTMCDIRQLLFVVEVVDKYFDCYDGGCINANQVIVNNLDKSAGFPFNKWGKARLYYDSMSYEDQDALFAY |
| | | TKRNVIPTITQMNLKYAISAKNRARTVAGVSICSTMTNRQFHQKLLKSIAATRGATVVIGTSKFYGGWHNMLKTVYSDVENP |
| | | HLMGWDYPKCDRAMPNMLRIMASLVLARKHTTCCSLSHRFYRLANECAQVLSEMVMCGGSLYVKPGGTSSGDATTAYAN |
| | | SVFNICQAVTANVNALLSTDGNKIADKYVRNLQHRLYECLYRNRDVDTDFVNEFYAYLRKHFSMMILSDDAVVCFNSTYAS |
| | | QGLVASIKNFKSVLYYQNNVFMSEAKCWTETDLTKGPHEFCSQHTMLVKQGDDYVYLPYPDPSRILGAGCFVDDIVKTDGT |
| | | LMIERFVSLAIDAYPLTKHPNQEYADVFHLYLQYIRKLHDELTGHMLDMYSVMLTNDNTSRYWEPEFYEAMYTPHTVLQGG |
| | | GGSGGGGSSKMSDVKCTSVVLLSVLQQLRVESSSKLWAQCVQLHNDILLAKDTTEAFEKMVSLLSVLLSMQGAVDINKLC |
| | | EEMLDNRATLQ |
| 291 | Concatemer | MGAVGACVLCNSQTSLRCGACIRRPFLCCKCCYDHVISTSHKLVLSVNPYVCNAPGCDVTDVTQLYLGGMSYYCKSHKPP |
| | (NSP13- | ISFPLCANGQVFGLYKNTCVGSDNVTDFNAIATCDWTNAGDYILANTCTERLKLFAAETLKATEETFKLSYGIATVREVLSDR |
| | G4furG4S- | ELHLSWEVGKPRPPLNRNYVFTGYRVTKNSKVQIGEYTFEKGDYGDAVVYRGTTTYKLNVGDYFVLTSHTVMPLSAPTLVP |
| | NSP12- | QEHYVRITGLYPTLNISDEFSSNVANYQKVGMQKYSTLQGPPGTGKSHFAIGLALYYPSARIVYTACSHAAVDALCEKALKY |
| | G4furG4S- | LPIDKCSRIIPARARVECFDKFKVNSTLEQYVFCTVNALPETTADIVVFDEISMATNYDLSVVNARLRAKHYVYIGDPAQLPAP |
| | NSP7) | RTLLTKGTLEPEYFNSVCRLMKTIGPDMFLGTCRRCPAEIVDTVSALVYDNKLKAHKDKSAQCFKMFYKGVITHDVSSAINR |
| | | PQIGVVREFLTRNPAWRKAVFISPYNSQNAVASKILGLPTQTVDSSQGSEYDYVIFTQTTETAHSCNVNRFNVAITRAKVGIL |
| | | CIMSDRDLYDKLQFTSLEIPRRNVATLQGGGGRSKRSGGGGSSADAQSFLNRVCGVSAARLTPCGTGTSTDVVYRAFDIY |
| | | NDKVAGFAKFLKTNCCRFQEKDEDDNLIDSYFVVKRHTFSNYQHEETIYNLLKDCPAVAKHDFFKFRIDGDMVPHISRQRLT |
| | | KYTMADLVYALRHFDEGNCDTLKEILVTYNCCDDDYFNKKDWYDFVENPDILRVYANLGERVRQALLKTVQFCDAMRNAGI |
| | | VGVLTLDNQDLNGNWYDFGDFIQTTPGSGVPVVDSYYSLLMPILTLTRALTAESHVDTDLTKPYIKWDLLKYDFTEERLKLF |
| | | DRYFKYWDQTYHPNCVNCLDDRCILHCANFNVLFSTVFPPTSFGPLVRKIFVDGVPFVVSTGYHFRELGVVHNQDVNLHSS |
| | | RLSFKELLVYAADPAMHAASGNLLLDKRTTCFSVAALTNNVAFQTVKPGNFNKDFYDFAVSKGFFKEGSSVELKHFFFAQD |
| | | GNAAISDYDYYRYNLPTMCDIRQLLFVVEVVDKYFDCYDGGCINANQVIVNNLDKSAGFPFNKWGKARLYYDSMSYEDQD |
| | | ALFAYTKRNVIPTITQMNLKYAISAKNRARTVAGVSICSTMTNRQFHQKLLKSIAATRGATVVIGTSKFYGGWHNMLKTVYSD |
| | | VENPHLMGWDYPKCDRAMPNMLRIMASLVLARKHTTCCSLSHRFYRLANECAQVLSEMVMCGGSLYVKPGGTSSGDATT |
| | | AYANSVFNICQAVTANVNALLSTDGNKIADKYVRNLQHRLYECLYRNRDVDTDFVNEFYAYLRKHFSMMILSDDAVVCFNS |
| | | TYASQGLVASIKNFKSVLYYQNNVFMSEAKCWTETDLTKGPHEFCSQHTMLVKQGDDYVYLPYPDPSRILGAGCFVDDIVK |
| | | TDGTLMIERFVSLAIDAYPLTKHPNQEYADVFHLYLQYIRKLHDELTGHMLDMYSVMLTNDNTSRYWEPEFYEAMYTPHTV |
| | | LQGGGGRSKRSGGGGSSKMSDVKCTSVVLLSVLQQLRVESSSKLWAQCVQLHNDILLAKDTTEAFEKMVSLLSVLLSMQ |
| | | GAVDINKLCEEMLDNRATLQ |
| 292 | Omicron- | ATGGGCGTTTTTCTTGTTTTATTGCCACTAGTCTCTAGTCAGTGTGTTAATCTTATAACCAGAACTCAATCATACACTAA |
| | BA52P | TTCTTTCACACGTGGTGTTTATTACCCTGACAAAGTTTTCAGATCCTCAGTTTTACATTCAACTCAGGACTTGTTCTTAC |
| | | CTTTCTTTTCCAATGTTACTTGGTTCCATGCTATCTCTGGGACCAATGGTATCAAGAGGTTTGATAACCCTGTCCTACC |
| | | ATTTAATGATGGTGTTTATTTTGCTTCCACTGAGAAGTCTAACATAATAAGAGGCTGGATTTTTGGTACTACTTTAGATT |
| | | CGAAGACCCAGTCCCTACTTATTGTTAATAACGCTACTAATGTTGTTATTAAAGTCTGTGAATTTCAATTTTGTAATGAT |
| | | CCATTTTTGGATGTTTATTACCACAAAAACAACAAAAGTTGGATGGAAAGTGAGTTCAGAGTTTATTCTAGTGCGAATA |
| | | ATTGCACTTTTGAATATGTCTCTCAGCCTTTTCTTATGGACCTTGAAGGAAAACAGGGTAATTTCAAAAATCTTAGGGAA |
| | | TTTGTGTTTAAGAATATTGATGGTTATTTTAAAATATATTCTAAGCACACGCCTATTAATTTAGGGCGTGATCTCCCTCA |
| | | GGGTTTTTCGGCTTTAGAACCATTGGTAGATTTGCCAATAGGTATTAACATCACTAGGTTTCAAACTTTACTTGCTTTAC |
| | | ATAGAAGTTATTTGACTCCTGGTGATTCTTCTTCAGGTTGGACAGCTGGTGCTGCAGCTTATTATGTGGGTTATCTTCA |
| | | ACCTAGGACTTTTCTATTAAAATATAATGAAAATGGAACCATTACAGATGCTGTAGACTGTGCACTTGACCCTCTCTCA |
| | | GAAACAAAGTGTACGTTGAAATCCTTCACTGTAGAAAAAGGAATCTATCAAACTTCTAACTTTAGAGTCCAACCAACAG |
| | | AATCTATTGTTAGATTTCCTAATATTACAAACTTGTGCCCTTTTGATGAAGTTTTTAACGCCACCAGATTTGCATCCGTG |
| | | TATGCTTGGAACAGGAAGAGAATCAGCAACTGTGTTGCTGATTATTCTGTCCTATATAATTTCGCACCATTTTTCGCTTT |
| | | TAAGTGTTATGGAGTGTCTCCTACTAAATTAAATGATCTCTGCTTTACTAATGTCTATGCAGATTCATTTGTAATTAGAG |
| | | GTAATGAAGTCAGCCAAATCGCTCCAGGGCAAACTGGAAATATTGCTGATTATAATTATAAATTACCAGATGATTTTAC |
| | | AGGCTGCGTTATAGCTTGGAATTCTAACAACCTTGATTCTAAGGTTGGTGGTAATTATAATTACCGGTATAGATTGTTTA |
| | | GGAAGTCTAATCTCAAACCTTTTGAGAGAGATATTTCAACTGAAATCTATCAGGCCGGTAACAAACCTTGTAATGGTGT |
| | | TGCAGGTGTTAATTGTTACTTTCCTTTACAATCATATGGTTTCCGACCCACTTATGGTGTTGGTCACCAACCATACAGA |
| | | GTAGTAGTACTTTCTTTTGAACTTCTACATGCACCAGCAACTGTTTGTGGACCTAAAAAGTCTACTAATTTGGTTAAAAA |
| | | CAAATGTGTCAATTTCAACTTCAATGGTTTAACAGGCACAGGTGTTCTTACTGAGTCTAACAAAAAGTTTCTGCCTTTCC |
| | | AACAATTTGGCAGAGACATTGCTGACACTACTGATGCTGTCCGTGATCCACAGACACTTGAGATTCTTGACATTACAC |
| | | CATGTTCTTTTGGTGGTGTCAGTGTTATAACACCAGGAACAAATACTTCTAACCAGGTTGCTGTTCTTTATCAGGGTGT |
| | | TAACTGCACAGAAGTCCCTGTTGCTATTCATGCAGATCAACTTACTCCTACTTGGCGTGTTTATTCTACAGGTTCTAAT |
| | | GTTTTTCAAACACGTGCAGGCTGTTTAATAGGGGCTGAATATGTCAACAACTCATATGAGTGTGACATACCCATTGGTG |
| | | CAGGTATATGCGCTAGTTATCAGACTCAGACTAAGTCTCATCGGCGGGCACGTAGTGTAGCTAGTCAATCCATCATTG |
| | | CCTACACTATGTCACTTGGTGCAGAAAATTCAGTTGCTTACTCTAATAACTCTATTGCCATACCCACAAATTTTACTATT |
| | | AGTGTTACCACAGAAATTCTACCAGTGTCTATGACCAAGACATCAGTAGATTGTACAATGTACATTTGTGGTGATTCAA |
| | | CTGAATGCAGCAATCTTTTGTTGCAATATGGCAGTTTTTGTACACAATTAAAACGTGCTTTAACTGGAATAGCTGTTGAA |
| | | CAAGACAAAAACACCCAAGAAGTTTTTGCACAAGTCAAACAAATTTACAAAACACCACCAATTAAATATTTTGGTGGTTT |
| | | TAATTTTTCACAAATATTACCAGATCCATCAAAACCAAGCAAGAGGTCATTTATTGAAGATCTACTTTTCAACAAAGTGA |
| | | CACTTGCAGATGCTGGCTTCATCAAACAATATGGTGATTGCCTTGGTGATATTGCTGCTAGAGACCTCATTTGTGCACA |
| | | AAAGTTTAACGGCCTTACTGTTTTGCCACCTTTGCTCACAGATGAAATGATTGCTCAATACACTTCTGCACTGTTAGCG |
| | | GGTACAATCACTTCTGGTTGGACCTTTGGTGCAGGTGCTGCATTACAAATACCATTTGCTATGCAAATGGCTTATAGGT |
| | | TTAATGGTATTGGAGTTACACAGAATGTTCTCTATGAGAACCAAAAATTGATTGCCAACCAATTTAATAGTGCTATTGGC |
| | | AAAATTCAAGACTCACTTTCTTCCACAGCAAGTGCACTTGGAAAACTTCAAGATGTGGTCAACCATAATGCACAAGCTT |
| | | TAAACACGCTTGTTAAACAACTTAGCTCCAAATTTGGTGCAATTTCAAGTGTTTTAAATGATATCCTTTCACGTCTTGAC |
| | | CCTCCCGAGGCTGAAGTGCAAATTGATAGGTTGATCACAGGCAGACTTCAAAGTTTGCAGACATATGTGACTCAACAA |
| | | TTAATTAGAGCTGCAGAAATCAGAGCTTCTGCTAATCTTGCTGCTACTAAAATGTCAGAGTGTGTACTTGGACAATCAA |
| | | AAAGAGTTGATTTTTGTGGAAAGGGCTATCATCTTATGTCCTTCCCTCAGTCAGCACCTCATGGTGTAGTCTTCTTGCA |
| | | TGTGACTTATGTCCCTGCACAAGAAAAGAACTTCACAACTGCTCCTGCCATTTGTCATGATGGAAAAGCACACTTTCCT |
| | | CGTGAAGGTGTCTTTGTTTCAAATGGCACACACTGGTTTGTAACACAAAGGAATTTTTATGAACCACAAATCATTACTA |
| | | CAGACAACACATTTGTGTCTGGTAACTGTGATGTTGTAATAGGAATTGTCAACAACACAGTTTATGATCCTTTGCAACC |
| | | TGAATTAGATTCATTCAAGGAGGAGTTAGATAAATATTTTAAGAATCATACATCACCAGATGTTGATTTAGGTGACATCT |
| | | CTGGCATTAATGCTTCAGTTGTAAACATTCAAAAAGAAATTGACCGCCTCAATGAGGTTGCCAAGAATTTAAATGAATC |
| | | TCTCATCGATCTCCAAGAACTTGGAAAGTATGAGCAGTATATAAAATGGCCATGGTACATTTGGCTAGGTTTTATAGCT |
| | | GGCTTGATTGCCATAGTAATGGTGACAATTATGCTTTGCTGTATGACCAGTTGCTGTAGTTGTCTCAAGGGCTGTTGTT |
| | | CTTGTGGATCCTGCTGCAAATTTGATGAAGACGACTCTGAGCCAGTGCTCAAAGGAGTCAAATTACATTACACA |
| 293 | Omicron- | ATGGGCGTTTTTCTTGTTTTATTGCCACTAGTCTCTAGTCAGTGTGTTAATCTTATCACCAGAACTCAAAGCTACACTAA |
| | BA52P | TTCTTTCACACGTGGTGTTTATTACCCTGACAAAGTTTTCAGATCCTCAGTTTTACATTCAACTCAGGATTTGTTCTTAC |
| | | CTTTCTTTTCCAATGTTACTTGGTTCCATGCTATATCTGGGACCAATGGTATCAAGAGGTTTGATAACCCTGTCCTACC |
| | | ATTTAATGATGGTGTTTATTTTGCTTCCACTGAGAAGTCTAACATAATAAGAGGCTGGATCTTTGGTACTACTTTAGATT |
| | | CGAAGACCCAGTCCCTACTTATTGTTAATAACGCTACTAATGTTGTTATTAAAGTCTGTGAATTTCAATTTTGTAATGAT |
| | | CCATTTTTGGACGTTTATTACCACAAAAACAACAAAAGTTGGATGGAAAGTGAGTTCAGAGTTTATTCTAGTGCGAATA |
| | | ATTGCACTTTTGAATATGTCTCTCAGCCTTTTCTTATGGACCTTGAAGGAAAACAGGGTAATTTCAAAAATCTTAGGGAA |
| | | TTTGTGTTTAAGAATATTGATGGTTATTTTAAAATATATTCTAAGCACACGCCTATTAATTTAGGCCGTGATCTCCCTCA |
| | | GGGTTTTTCGGCTTTAGAACCATTGGTAGATTTGCCAATAGGTATTAACATCACTAGGTTTCAAACTTTACTTGCTTTAC |
| | | ATAGAAGTTATTTGACTCCTGGTGATTCTTCTTCAGGTTGGACAGCTGGTGCTGCAGCTTATTATGTGGGTTATCTTCA |
| | | ACCTAGGACTTTTCTATTAAAATATAATGAAAATGGAACCATTACAGATGCTGTAGACTGTGCACTTGACCCTCTCTCA |
| | | GAAACAAAGTGTACGTTGAAATCCTTCACTGTAGAAAAAGGAATCTATCAAACTTCTAACTTTAGAGTCCAACCAACAG |
| | | AATCTATTGTTAGATTTCCTAATATTACAAACTTGTGCCCTTTTGACGAAGTTTTTAACGCCACCAGATTTGCATCCGTG |
| | | TATGCTTGGAACAGGAAGAGAATCAGCAACTGTGTTGCTGATTATTCTGTCCTATATAATTTCGCACCCTTTTTCGCCT |
| | | TTAAGTGTTATGGAGTGTCTCCTACTAAATTAAATGATCTCTGCTTTACTAATGTCTATGCAGATTCATTTGTAATTAGA |
| | | GGTAACGAAGTCAGCCAAATCGCTCCAGGGCAAACTGGAAATATTGCTGATTATAATTATAAATTACCAGATGATTTTA |
| | | CAGGCTGCGTTATAGCTTGGAATTCTAACAATCTTGATTCTAAGGTTGGTGGTAATTATAATTACCGGTATAGATTGTTT |
| | | AGGAAGTCTAATCTCAAACCTTTTGAGAGAGATATTTCAACTGAAATCTATCAGGCCGGTAACAAGCCTTGTAATGGTG |
| | | TTGCCGGTGTGAATTGTTACTTTCCTTTACAATCATATGGTTTCCGGCCCACTTACGGTGTTGGTCACCAACCATACAG |
| | | AGTAGTAGTACTTTCTTTTGAACTTCTACATGCACCAGCAACTGTTTGTGGACCTAAAAAGTCTACTAATTTGGTTAAAA |
| | | ACAAATGTGTCAATTTCAACTTCAATGGTTTAACAGGCACAGGTGTTCTTACTGAGTCTAACAAAAAGTTTCTGCCTTTC |
| | | CAACAATTTGGCAGAGACATTGCTGACACTACTGATGCTGTCCGTGATCCACAGACACTTGAGATTCTTGACATTACA |
| | | CCATGTTCTTTTGGTGGTGTCAGTGTTATAACACCAGGAACAAATACTTCTAACCAGGTTGCTGTTCTTTATCAGGGCG |
| | | TTAACTGCACAGAAGTCCCTGTTGCTATTCATGCAGATCAACTTACTCCTACTTGGCGTGTTTATTCTACAGGTTCTAAT |
| | | GTTTTTCAAACACGTGCAGGCTGTTTAATAGGGGCTGAATACGTCAACAACTCATATGAGTGTGACATACCCATTGGT |
| | | GCAGGTATATGCGCTAGTTATCAGACTCAGACTAAGTCTCACCGGGGGGCACGTAGTGTAGCTAGTCAATCCATCATT |
| | | GCCTACACTATGTCACTTGGTGCAGAAAATTCAGTTGCTTACTCTAATAACTCTATTGCCATACCCACAAATTTTACTAT |
| | | TAGCGTGACCACTGAAATCCTCCCCGTGAGCATGACCAAGACCAGCGTGGACTGCACCATGTACATCTGCGGCGACA |
| | | GCACCGAGTGCAGCAACCTGCTCCTGCAGTACGGCAGCTTCTGCACCCAGCTGAAGCGTGCTTTAACTGGGATAGCT |
| | | GTTGAACAAGACAAAAACACCCAAGAAGTTTTTGCACAAGTCAAACAAATTTACAAAACACCACCAATTAAATACTTTG |
| | | GTGGTTTTAATTTTTCACAAATATTACCAGATCCATCAAAACCAAGCAAGAGGTCATTTATTGAAGATCTACTTTTCAAC |
| | | AAAGTGACACTTGCAGATGCTGGCTTCATCAAACAATATGGTGATTGCCTTGGTGATATTGCTGCTAGGGACCTCATTT |
| | | GTGCACAAAAGTTTAACGGCCTTACTGTTTTGCCACCTTTGCTCACAGATGAAATGATTGCTCAATACACTTCTGCACT |
| | | GTTAGCGGGTACAATCACTTCTGGTTGGACCTTTGGTGCAGGTGCTGCATTACAAATACCATTTGCTATGCAAATGGC |
| | | TTATAGGTTTAATGGTATTGGAGTTACACAGAATGTTCTCTATGAGAACCAAAAATTGATTGCCAACCAATTTAATAGTG |
| | | CCATTGGCAAAATTCAAGACTCACTTTCTTCCACAGCAAGTGCACTTGGAAAACTTCAAGATGTGGTCAACCACAATGC |
| | | ACAAGCTTTAAACACGCTTGTTAAACAACTTAGCTCCAAGTTTGGTGCAATTTCAAGTGTTTTAAATGATATCCTTTCAC |
| | | GTCTTGACCCTCCCGAGGCTGAAGTGCAAATTGATAGGTTGATCACAGGCAGACTTCAAAGTTTGCAGACATATGTGA |
| | | CTCAACAATTAATTAGAGCTGCAGAAATCAGAGCTTCTGCTAATCTTGCTGCTACTAAAATGTCAGAGTGTGTACTTGG |
| | | ACAATCAAAAAGAGTTGATTTTTGTGGAAAGGGCTATCATCTTATGTCCTTCCCTCAGTCAGCACCTCATGGTGTAGTC |
| | | TTCTTGCATGTGACTTATGTCCCTGCACAAGAAAAGAACTTCACAACTGCTCCTGCCATTTGTCATGATGGAAAAGCAC |
| | | ACTTTCCTCGTGAAGGTGTCTTTGTTTCAAATGGCACACACTGGTTTGTAACACAAAGGAATTTTTATGAACCACAAAT |
| | | CATTACTACAGACAACACATTTGTGTCTGGTAACTGTGATGTTGTAATAGGAATTGTCAACAACACAGTTTATGATCCTT |
| | | TGCAACCTGAATTAGACTCATTCAAGGAGGAGTTAGATAAATATTTTAAGAATCATACATCACCAGATGTTGATTTAGGT |
| | | GACATCTCTGGCATTAATGCTTCAGTTGTAAACATTCAAAAAGAAATTGACCGCCTCAATGAGGTTGCCAAGAATTTAA |
| | | ATGAATCTCTCATCGATCTCCAAGAACTTGGAAAGTATGAGCAGTATATAAAATGGCCATGGTACATTTGGCTAGGTTT |
| | | TATAGCTGGCTTGATTGCCATAGTAATGGTGACAATTATGCTTTGCTGTATGACCAGTTGCTGTAGTTGTCTCAAGGGC |
| | | TGTTGTTCTTGTGGATCCTGCTGCAAATTTGATGAAGACGACTCTGAGCCAGTGCTCAAAGGAGTCAAATTACATTACA |
| | | CA |
| 294 | Spike-_ | ATGTTTGTTTTTCTTGTTTTATTGCCACTAGTCTCTAGTCAGTGTGTTAATCTTACAACCAGAACTCAATTACCCCCTGC |
| | HexaP | ATACACTAATTCTTTCACACGTGGTGTTTATTACCCTGACAAAGTTTTCAGATCCTCAGTTTTACATTCAACTCAGGATT |
| | | TGTTCTTACCTTTCTTTTCCAATGTTACTTGGTTCCATGCTATACATGTCTCTGGGACCAATGGTACTAAGAGGTTTGAT |
| | | AACCCTGTCCTACCATTTAATGATGGTGTTTATTTTGCTTCCACTGAGAAGTCTAACATAATAAGAGGCTGGATCTTTG |
| | | GTACTACTTTAGATTCGAAGACCCAGTCCCTACTTATTGTTAATAACGCTACTAATGTTGTTATTAAAGTCTGTGAATTT |
| | | CAATTTTGTAATGATCCATTTTTGGGTGTTTATTACCACAAAAACAACAAAAGTTGGATGGAAAGTGAGTTCAGAGTTTA |
| | | TTCTAGTGCGAATAATTGCACTTTTGAATATGTCTCTCAGCCTTTTCTTATGGACCTTGAAGGAAAACAGGGTAATTTCA |
| | | AAAATCTTAGGGAATTTGTGTTTAAGAATATTGATGGTTATTTTAAAATATATTCTAAGCACACGCCTATTAATTTAGTGC |
| | | GTGATCTCCCTCAGGGTTTTTCGGCTTTAGAACCATTGGTAGATTTGCCAATAGGTATTAACATCACTAGGTTTCAAAC |
| | | TTTACTTGCTTTACATAGAAGTTATTTGACTCCTGGTGATTCTTCTTCAGGTTGGACAGCTGGTGCTGCAGCTTATTAT |
| | | GTGGGTTATCTTCAACCTAGGACTTTTCTATTAAAATATAATGAAAATGGAACCATTACAGATGCTGTAGACTGTGCAC |
| | | TTGACCCTCTCTCAGAAACAAAGTGTACGTTGAAATCCTTCACTGTAGAAAAAGGAATCTATCAAACTTCTAACTTTAGA |
| | | GTCCAACCAACAGAATCTATTGTTAGATTTCCTAATATTACAAACTTGTGCCCTTTTGGTGAAGTTTTTAACGCCACCAG |
| | | ATTTGCATCCGTGTATGCTTGGAACAGGAAGAGAATCAGCAACTGTGTTGCTGATTATTCTGTCCTATATAATTCCGCA |
| | | TCATTTTCCACTTTTAAGTGTTATGGAGTGTCTCCTACTAAATTAAATGATCTCTGCTTTACTAATGTCTATGCAGATTCA |
| | | TTTGTAATTAGAGGTGATGAAGTCAGACAAATCGCTCCAGGGCAAACTGGAAAGATTGCTGATTATAATTATAAATTAC |
| | | CAGATGATTTTACAGGCTGCGTTATAGCTTGGAATTCTAACAATCTTGATTCTAAGGTTGGTGGTAATTATAATTACCTG |
| | | TATAGATTGTTTAGGAAGTCTAATCTCAAACCTTTTGAGAGAGATATTTCAACTGAAATCTATCAGGCCGGTAGCACAC |
| | | CTTGTAATGGTGTTGAAGGTTTTAATTGTTACTTTCCTTTACAATCATATGGTTTCCAACCCACTAATGGTGTTGGTTAC |
| | | CAACCATACAGAGTAGTAGTACTTTCTTTTGAACTTCTACATGCACCAGCAACTGTTTGTGGACCTAAAAAGTCTACTA |
| | | ATTTGGTTAAAAACAAATGTGTCAATTTCAACTTCAATGGTTTAACAGGCACAGGTGTTCTTACTGAGTCTAACAAAAAG |
| | | TTTCTGCCTTTCCAACAATTTGGCAGAGACATTGCTGACACTACTGATGCTGTCCGTGATCCACAGACACTTGAGATTC |
| | | TTGACATTACACCATGTTCTTTTGGTGGTGTCAGTGTTATAACACCAGGAACAAATACTTCTAACCAGGTTGCTGTTCT |
| | | TTATCAGGATGTTAACTGCACAGAAGTCCCTGTTGCTATTCATGCAGATCAACTTACTCCTACTTGGCGTGTTTATTCT |
| | | ACAGGTTCTAATGTTTTTCAAACACGTGCAGGCTGTTTAATAGGGGCTGAACATGTCAACAACTCATATGAGTGTGACA |
| | | TACCCATTGGTGCAGGTATATGCGCTAGTTATCAGACTCAGACTAATTCTCCTCGGCGGGCACGTAGTGTAGCTAGTC |
| | | AATCCATCATTGCCTACACTATGTCACTTGGTGCAGAAAATTCAGTTGCTTACTCTAATAACTCTATTGCCATACCCACA |
| | | AATTTTACTATTAGTGTTACCACAGAAATTCTACCAGTGTCTATGACCAAGACATCAGTAGATTGTACAATGTACATTTG |
| | | TGGTGATTCAACTGAATGCAGCAATCTTTTGTTGCAATATGGCAGTTTTTGTACACAATTAAACCGTGCTTTAACTGGG |
| | | ATAGCTGTTGAACAAGACAAAAACACCCAAGAAGTTTTTGCACAAGTCAAACAAATTTACAAAACACCACCAATTAAAG |
| | | ATTTTGGTGGTTTTAATTTTTCACAAATATTACCAGATCCATCAAAACCAAGCAAGAGGTCACCTATTGAAGATCTACTT |
| | | TTCAACAAAGTGACACTTGCAGATGCTGGCTTCATCAAACAATATGGTGATTGCCTTGGTGATATTGCTGCTAGGGAC |
| | | CTCATTTGTGCACAAAAGTTTAACGGCCTTACTGTTTTGCCACCTTTGCTCACAGATGAAATGATTGCTCAATACACTT |
| | | CTGCACTGTTAGCGGGTACAATCACTTCTGGTTGGACCTTTGGTGCAGGTCCTGCATTACAAATACCATTTCCTATGC |
| | | AAATGGCTTATAGGTTTAATGGTATTGGAGTTACACAGAATGTTCTCTATGAGAACCAAAAATTGATTGCCAACCAATTT |
| | | AATAGTGCCATTGGCAAAATTCAAGACTCACTTTCTTCCACACCTAGTGCACTTGGAAAACTTCAAGATGTGGTCAACC |
| | | AAAATGCACAAGCTTTAAACACGCTTGTTAAACAACTTAGCTCCAATTTTGGTGCAATTTCAAGTGTTTTAAATGATATC |
| | | CTTTCACGTCTTGACCCTCCCGAGGCTGAAGTGCAAATTGATAGGTTGATCACAGGCAGACTTCAAAGTTTGCAGACA |
| | | TATGTGACTCAACAATTAATTAGAGCTGCAGAAATCAGAGCTTCTGCTAATCTTGCTGCTACTAAAATGTCAGAGTGTG |
| | | TACTTGGACAATCAAAAAGAGTTGATTTTTGTGGAAAGGGCTATCATCTTATGTCCTTCCCTCAGTCAGCACCTCATGG |
| | | TGTAGTCTTCTTGCATGTGACTTATGTCCCTGCACAAGAAAAGAACTTCACAACTGCTCCTGCCATTTGTCATGATGGA |
| | | AAAGCACACTTTCCTCGTGAAGGTGTCTTTGTTTCAAATGGCACACACTGGTTTGTAACACAAAGGAATTTTTATGAAC |
| | | CACAAATCATTACTACAGACAACACATTTGTGTCTGGTAACTGTGATGTTGTAATAGGAATTGTCAACAACACAGTTTAT |
| | | GATCCTTTGCAACCTGAATTAGACTCATTCAAGGAGGAGTTAGATAAATATTTTAAGAATCATACATCACCAGATGTTG |
| | | ATTTAGGTGACATCTCTGGCATTAATGCTTCAGTTGTAAACATTCAAAAAGAAATTGACCGCCTCAATGAGGTTGCCAA |
| | | GAATTTAAATGAATCTCTCATCGATCTCCAAGAACTTGGAAAGTATGAGCAGTATATAAAATGGCCATGGTACATTTGG |
| | | CTAGGTTTTATAGCTGGCTTGATTGCCATAGTAATGGTGACAATTATGCTTTGCTGTATGACCAGTTGCTGTAGTTGTC |
| | | TCAAGGGCTGTTGTTCTTGTGGATCCTGCTGCAAATTTGATGAAGACGACTCTGAGCCAGTGCTCAAAGGAGTCAAAT |
| | | TACATTACACA |
| 295 | BA5-Spike- | ATGGGCGTTTTTCTTGTTTTATTGCCACTAGTCTCTAGTCAGTGTGTTAATCTTATCACCAGAACTCAAAGCTACACTAA |
| | HexaP | TTCTTTCACACGTGGTGTTTATTACCCTGACAAAGTTTTCAGATCCTCAGTTTTACATTCAACTCAGGATTTGTTCTTAC |
| | | CTTTCTTTTCCAATGTTACTTGGTTCCATGCTATATCTGGGACCAATGGTATCAAGAGGTTTGATAACCCTGTCCTACC |
| | | ATTTAATGATGGTGTTTATTTTGCTTCCACTGAGAAGTCTAACATAATAAGAGGCTGGATCTTTGGTACTACTTTAGATT |
| | | CGAAGACCCAGTCCCTACTTATTGTTAATAACGCTACTAATGTTGTTATTAAAGTCTGTGAATTTCAATTTTGTAATGAT |
| | | CCATTTTTGGACGTTTATTACCACAAAAACAACAAAAGTTGGATGGAAAGTGAGTTCAGAGTTTATTCTAGTGCGAATA |
| | | ATTGCACTTTTGAATATGTCTCTCAGCCTTTTCTTATGGACCTTGAAGGAAAACAGGGTAATTTCAAAAATCTTAGGGAA |
| | | TTTGTGTTTAAGAATATTGATGGTTATTTTAAAATATATTCTAAGCACACGCCTATTAATTTAGGCCGTGATCTCCCTCA |
| | | GGGTTTTTCGGCTTTAGAACCATTGGTAGATTTGCCAATAGGTATTAACATCACTAGGTTTCAAACTTTACTTGCTTTAC |
| | | ATAGAAGTTATTTGACTCCTGGTGATTCTTCTTCAGGTTGGACAGCTGGTGCTGCAGCTTATTATGTGGGTTATCTTCA |
| | | ACCTAGGACTTTTCTATTAAAATATAATGAAAATGGAACCATTACAGATGCTGTAGACTGTGCACTTGACCCTCTCTCA |
| | | GAAACAAAGTGTACGTTGAAATCCTTCACTGTAGAAAAAGGAATCTATCAAACTTCTAACTTTAGAGTCCAACCAACAG |
| | | AATCTATTGTTAGATTTCCTAATATTACAAACTTGTGCCCTTTTGACGAAGTTTTTAACGCCACCAGATTTGCATCCGTG |
| | | TATGCTTGGAACAGGAAGAGAATCAGCAACTGTGTTGCTGATTATTCTGTCCTATATAATTTCGCACCCTTTTTCGCCT |
| | | TTAAGTGTTATGGAGTGTCTCCTACTAAATTAAATGATCTCTGCTTTACTAATGTCTATGCAGATTCATTTGTAATTAGA |
| | | GGTAACGAAGTCAGCCAAATCGCTCCAGGGCAAACTGGAAATATTGCTGATTATAATTATAAATTACCAGATGATTTTA |
| | | CAGGCTGCGTTATAGCTTGGAATTCTAACAATCTTGATTCTAAGGTTGGTGGTAATTATAATTACCGGTATAGATTGTTT |
| | | AGGAAGTCTAATCTCAAACCTTTTGAGAGAGATATTTCAACTGAAATCTATCAGGCCGGTAACAAGCCTTGTAATGGTG |
| | | TTGCCGGTGTGAATTGTTACTTTCCTTTACAATCATATGGTTTCCGGCCCACTTACGGTGTTGGTCACCAACCATACAG |
| | | AGTAGTAGTACTTTCTTTTGAACTTCTACATGCACCAGCAACTGTTTGTGGACCTAAAAAGTCTACTAATTTGGTTAAAA |
| | | ACAAATGTGTCAATTTCAACTTCAATGGTTTAACAGGCACAGGTGTTCTTACTGAGTCTAACAAAAAGTTTCTGCCTTTC |
| | | CAACAATTTGGCAGAGACATTGCTGACACTACTGATGCTGTCCGTGATCCACAGACACTTGAGATTCTTGACATTACA |
| | | CCATGTTCTTTTGGTGGTGTCAGTGTTATAACACCAGGAACAAATACTTCTAACCAGGTTGCTGTTCTTTATCAGGGCG |
| | | TTAACTGCACAGAAGTCCCTGTTGCTATTCATGCAGATCAACTTACTCCTACTTGGCGTGTTTATTCTACAGGTTCTAAT |
| | | GTTTTTCAAACACGTGCAGGCTGTTTAATAGGGGCTGAATACGTCAACAACTCATATGAGTGTGACATACCCATTGGT |
| | | GCAGGTATATGCGCTAGTTATCAGACTCAGACTAAGTCTCACCGGCGGGCACGTAGTGTAGCTAGTCAATCCATCATT |
| | | GCCTACACTATGTCACTTGGTGCAGAAAATTCAGTTGCTTACTCTAATAACTCTATTGCCATACCCACAAATTTTACTAT |
| | | TAGCGTGACCACTGAAATCCTCCCCGTGAGCATGACCAAGACCAGCGTGGACTGCACCATGTACATCTGCGGCGACA |
| | | GCACCGAGTGCAGCAACCTGCTCCTGCAGTACGGCAGCTTCTGCACCCAGCTGAAGCGTGCTTTAACTGGGATAGCT |
| | | GTTGAACAAGACAAAAACACCCAAGAAGTTTTTGCACAAGTCAAACAAATTTACAAAACACCACCAATTAAATACTTTG |
| | | GTGGTTTTAATTTTTCACAAATATTACCAGATCCATCAAAACCAAGCAAGAGGTCACCTATTGAAGATCTACTTTTCAAC |
| | | AAAGTGACACTTGCAGATGCTGGCTTCATCAAACAATATGGTGATTGCCTTGGTGATATTGCTGCTAGGGACCTCATTT |
| | | GTGCACAAAAGTTTAACGGCCTTACTGTTTTGCCACCTTTGCTCACAGATGAAATGATTGCTCAATACACTTCTGCACT |
| | | GTTAGCGGGTACAATCACTTCTGGTTGGACCTTTGGTGCAGGTCCTGCATTACAAATACCATTTCCTATGCAAATGGC |
| | | TTATAGGTTTAATGGTATTGGAGTTACACAGAATGTTCTCTATGAGAACCAAAAATTGATTGCCAACCAATTTAATAGTG |
| | | CCATTGGCAAAATTCAAGACTCACTTTCTTCCACACCTAGTGCACTTGGAAAACTTCAAGATGTGGTCAACCACAATGC |
| | | ACAAGCTTTAAACACGCTTGTTAAACAACTTAGCTCCAAGTTTGGTGCAATTTCAAGTGTTTTAAATGATATCCTTTCAC |
| | | GTCTTGACCCTCCCGAGGCTGAAGTGCAAATTGATAGGTTGATCACAGGCAGACTTCAAAGTTTGCAGACATATGTGA |
| | | CTCAACAATTAATTAGAGCTGCAGAAATCAGAGCTTCTGCTAATCTTGCTGCTACTAAAATGTCAGAGTGTGTACTTGG |
| | | ACAATCAAAAAGAGTTGATTTTTGTGGAAAGGGCTATCATCTTATGTCCTTCCCTCAGTCAGCACCTCATGGTGTAGTC |
| | | TTCTTGCATGTGACTTATGTCCCTGCACAAGAAAAGAACTTCACAACTGCTCCTGCCATTTGTCATGATGGAAAAGCAC |
| | | ACTTTCCTCGTGAAGGTGTCTTTGTTTCAAATGGCACACACTGGTTTGTAACACAAAGGAATTTTTATGAACCACAAAT |
| | | CATTACTACAGACAACACATTTGTGTCTGGTAACTGTGATGTTGTAATAGGAATTGTCAACAACACAGTTTATGATCCTT |
| | | TGCAACCTGAATTAGACTCATTCAAGGAGGAGTTAGATAAATATTTTAAGAATCATACATCACCAGATGTTGATTTAGGT |
| | | GACATCTCTGGCATTAATGCTTCAGTTGTAAACATTCAAAAAGAAATTGACCGCCTCAATGAGGTTGCCAAGAATTTAA |
| | | ATGAATCTCTCATCGATCTCCAAGAACTTGGAAAGTATGAGCAGTATATAAAATGGCCATGGTACATTTGGCTAGGTTT |
| | | TATAGCTGGCTTGATTGCCATAGTAATGGTGACAATTATGCTTTGCTGTATGACCAGTTGCTGTAGTTGTCTCAAGGGC |
| | | TGTTGTTCTTGTGGATCCTGCTGCAAATTTGATGAAGACGACTCTGAGCCAGTGCTCAAAGGAGTCAAATTACATTACA |
| | | CA |
| 296 | Secretion | ATGGGCGTGACCGCCCCCCGGACCCTGATCCTGCTCCTGAGCGGCGCCCTGGCCCTGACCGAAACCTGGGCCGGC |
| | signal | AGCGCCGTGGGCGCCTGCGTGCTGTGTAACAGTCAGACCAGCCTGCGGTGCGGCGCCTGCATCCGCAGGCCCTTC |
| | sequence | CTGTGCTGTAAGTGCTGCTACGACCACGTGATTAGCACAAGCCACAAGCTCGTGCTCAGCGTGAACCCCTACGTGTG |
| | (under- | CAACGCCCCCGGCTGCGACGTGACAGACGTGACCCAGCTGTACCTGGGCGGAATGAGCTACTATTGTAAGAGTCAC |
| | lined) | AAGCCCCCTATCAGCTTCCCCCTGTGCGCCAACGGCCAGGTGTTCGGCCTGTACAAGAACACCTGCGTGGGCAGCG |
| | NSP13 | ACAACGTGACCGACTTCAACGCCATCGCCACCTGCGACTGGACCAACGCCGGCGACTACATCCTGGCCAACACCTG |
| | | CACCGAGCGGCTGAAGCTGTTCGCCGCTGAAACACTGAAGGCCACCGAGGAAACCTTCAAGCTGAGCTACGGCATC |
| | | GCCACCGTGCGGGAGGTGCTCAGCGATCGGGAACTGCACCTGAGCTGGGAGGTGGGCAAGCCCCGGCCTCCTCTG |
| | | AACCGGAACTACGTGTTCACCGGCTACCGGGTGACCAAGAACAGCAAGGTGCAGATCGGCGAGTACACCTTCGAGA |
| | | AGGGCGACTACGGCGACGCCGTGGTCTACCGGGGCACCACAACCTACAAGCTGAACGTGGGCGACTACTTCGTGCT |
| | | GACCAGCCACACCGTGATGCCCCTGAGCGCCCCCACCCTGGTGCCCCAGGAACACTACGTGCGGATCACCGGCCTG |
| | | TACCCCACCCTGAACATCAGCGACGAGTTCAGCTCTAACGTCGCCAACTACCAGAAAGTGGGAATGCAGAAGTATAG |
| | | CACACTGCAGGGCCCCCCAGGCACCGGCAAGAGCCACTTCGCCATCGGCCTGGCCCTGTACTACCCCAGCGCCCG |
| | | GATCGTGTACACCGCCTGCAGCCACGCCGCCGTGGACGCCCTGTGCGAGAAGGCCCTGAAGTACCTGCCCATCGAC |
| | | AAGTGCAGCCGGATCATTCCCGCTCGGGCTCGGGTGGAGTGCTTCGACAAGTTCAAGGTGAACAGTACCCTCGAGC |
| | | AGTACGTGTTCTGCACCGTGAACGCCCTGCCCGAAACAACTGCCGACATCGTGGTGTTCGACGAGATCAGCATGGCC |
| | | ACCAACTACGACCTGAGCGTGGTTAACGCCCGGCTGCGGGCCAAGCACTACGTGTACATCGGCGACCCCGCCCAGC |
| | | TGCCCGCCCCCCGGACCCTGCTCACCAAGGGCACCCTGGAGCCCGAGTACTTCAACAGCGTGTGCCGGCTGATGAA |
| | | GACCATCGGCCCCGACATGTTCCTGGGCACCTGCCGGCGGTGCCCCGCCGAGATCGTGGACACCGTGAGCGCCCT |
| | | GGTGTACGACAACAAGCTGAAGGCCCACAAGGACAAGAGCGCCCAGTGCTTCAAGATGTTCTACAAGGGCGTGATCA |
| | | CCCACGACGTGAGCAGCGCCATCAACCGGCCCCAGATCGGCGTGGTGCGGGAGTTCCTGACCCGGAACCCCGCCT |
| | | GGCGGAAGGCCGTGTTCATCAGCCCCTACAACAGCCAGAACGCCGTGGCCAGCAAGATCCTGGGCCTGCCCACCCA |
| | | GACCGTGGACAGCTCTCAGGGCAGCGAGTACGACTACGTGATCTTCACCCAGACCACCGAAACAGCCCACAGCTGC |
| | | AACGTCAACCGCTTCAACGTGGCCATCACCCGGGCCAAGGTGGGCATCCTGTGCATCATGAGCGACCGGGACCTGT |
| | | ACGACAAGCTGCAGTTCACCAGCCTGGAGATCCCCCGGAGGAACGTGGCCACCCTGCAG |
| 297 | Secretion | ATGGGCGTGACCGCCCCCCGGACCCTGATCCTGCTCCTGAGCGGCGCCCTGGCCCTGACCGAAACCTGGGCCGGC |
| | signal | AGCAGCGCCGACGCCCAGAGCTTCCTGAACCGGGTGTGCGGCGTGAGCGCCGCTCGGCTGACCCCTTGCGGAACC |
| | sequence | GGAACCAGTACCGACGTGGTCTACCGGGCCTTCGACATCTACAACGACAAGGTGGCCGGCTTCGCCAAGTTCCTGAA |
| | (under- | GACCAACTGCTGTCGGTTCCAGGAGAAGGACGAGGACGATAACCTGATCGACAGCTATTTCGTCGTGAAGCGGCACA |
| | lined) | CCTTCAGCAACTACCAGCACGAGGAAACCATCTACAACCTGCTCAAGGACTGCCCCGCCGTGGCCAAGCACGACTTC |
| | NSP12 | TTTAAGTTCCGGATCGACGGCGACATGGTGCCCCACATCAGTCGGCAACGGCTGACCAAGTACACCATGGCCGACCT |
| | | CGTGTACGCCCTGCGGCACTTCGACGAGGGCAACTGCGACACCCTGAAGGAGATCCTGGTGACCTACAACTGCTGC |
| | | GACGACGACTATTTCAATAAGAAAGATTGGTACGATTTTGTGGAAAACCCTGACATCCTGCGGGTGTACGCCAACCTG |
| | | GGCGAGCGGGTGCGGCAGGCCCTCCTGAAAACCGTCCAGTTCTGCGACGCCATGCGGAACGCCGGCATCGTGGGC |
| | | GTGCTGACCCTGGATAACCAAGACCTGAACGGCAACTGGTACGACTTCGGCGACTTCATCCAGACCACACCCGGCAG |
| | | CGGCGTGCCCGTGGTTGACAGCTACTATAGCCTGTTGATGCCCATCCTGACCCTGACCCGGGCCCTGACCGCCGAG |
| | | AGCCACGTGGATACCGATCTGACAAAGCCCTACATCAAGTGGGACCTGCTGAAGTACGACTTCACCGAGGAGCGGCT |
| | | GAAGCTGTTCGACCGGTACTTCAAGTACTGGGACCAGACCTACCACCCCAACTGCGTGAACTGCCTGGACGACCGGT |
| | | GCATCCTGCACTGCGCCAACTTCAACGTGCTGTTCAGCACCGTGTTCCCCCCTACCAGCTTCGGCCCCCTGGTGCGG |
| | | AAGATTTTCGTCGACGGCGTGCCCTTCGTGGTGAGCACCGGCTACCACTTCCGGGAGCTGGGCGTGGTGCACAACC |
| | | AGGACGTGAACCTGCACAGCTCCCGGCTGAGCTTCAAGGAGCTGCTGGTGTACGCCGCCGACCCCGCCATGCACGC |
| | | CGCAAGCGGCAACCTGCTCCTGGACAAGCGGACCACCTGCTTCAGCGTGGCCGCCCTGACCAACAACGTGGCCTTC |
| | | CAGACAGTGAAACCCGGAAACTTCAACAAGGACTTCTACGACTTCGCCGTGAGCAAGGGCTTCTTCAAGGAGGGCAG |
| | | CAGCGTGGAGCTGAAGCACTTCTTCTTCGCCCAGGACGGCAACGCTGCCATTAGCGACTACGACTACTACCGGTACA |
| | | ACCTGCCCACCATGTGCGACATCCGGCAGCTGTTGTTCGTGGTCGAGGTGGTGGACAAGTACTTCGACTGCTACGAC |
| | | GGCGGCTGCATCAACGCCAACCAGGTGATCGTGAACAACCTGGACAAGAGCGCCGGCTTCCCCTTCAACAAGTGGG |
| | | GCAAGGCCCGGCTGTACTACGACAGCATGAGCTACGAGGACCAGGACGCCCTGTTCGCCTACACCAAGCGGAACGT |
| | | GATCCCCACCATCACCCAGATGAACCTGAAGTACGCCATCAGCGCCAAGAACCGGGCCCGGACCGTGGCCGGCGTG |
| | | AGCATCTGCAGCACCATGACCAACCGGCAGTTCCACCAGAAGCTGCTCAAAAGTATCGCCGCTACCCGGGGCGCCA |
| | | CCGTGGTTATCGGCACCAGCAAGTTCTACGGCGGCTGGCACAACATGCTGAAGACCGTGTACAGCGACGTGGAGAA |
| | | CCCCCACCTGATGGGCTGGGACTACCCCAAGTGCGACCGGGCCATGCCCAACATGCTGCGGATCATGGCCAGCCTG |
| | | GTGCTGGCCCGCAAGCATACCACCTGCTGCAGCCTGAGCCACCGGTTCTACCGGCTGGCCAACGAGTGCGCCCAGG |
| | | TGCTGAGCGAGATGGTGATGTGCGGCGGGAGCCTGTACGTGAAGCCCGGCGGCACCAGCTCTGGCGACGCCACCA |
| | | CCGCCTACGCCAACAGCGTGTTCAACATCTGCCAGGCCGTGACCGCCAACGTGAACGCCCTGCTCAGCACCGACGG |
| | | CAACAAGATCGCCGACAAGTACGTGCGGAACCTGCAGCACCGGCTGTACGAGTGCCTGTACCGGAACCGGGACGTG |
| | | GACACCGACTTCGTGAACGAGTTTTACGCCTACCTGCGGAAGCACTTCAGCATGATGATCCTGAGCGACGACGCCGT |
| | | GGTGTGCTTCAACAGCACCTACGCCAGCCAGGGCCTGGTGGCCAGCATCAAGAACTTCAAGAGCGTGCTGTACTACC |
| | | AGAACAACGTGTTCATGAGCGAGGCCAAGTGCTGGACCGAAACAGACCTGACCAAGGGCCCCCACGAGTTCTGCAG |
| | | CCAGCACACCATGCTGGTGAAGCAGGGCGACGATTACGTGTACCTGCCCTACCCCGACCCCAGCCGGATCCTGGGC |
| | | GCCGGCTGCTTCGTGGACGACATCGTGAAGACCGACGGCACCCTGATGATCGAGCGGTTCGTGAGCCTGGCCATCG |
| | | ACGCCTACCCCCTGACCAAGCACCCCAACCAGGAGTACGCCGACGTGTTCCACCTGTACCTGCAGTACATCCGGAAG |
| | | CTGCACGACGAGCTGACCGGCCACATGCTGGACATGTACAGCGTGATGCTGACCAACGACAACACCAGCCGGTACT |
| | | GGGAGCCCGAGTTCTACGAGGCCATGTACACCCCCCACACCGTGCTGCAG |
| 298 | Secretion | ATGGGCGTGACCGCCCCCCGGACCCTGATCCTGCTCCTGAGCGGCGCCCTGGCCCTGACCGAAACCTGGGCCGGC |
| | signal | AGCAGCAAGATGAGCGACGTGAAGTGCACCAGCGTGGTCCTGCTCAGCGTGCTGCAGCAACTGCGGGTGGAGAGCT |
| | sequence | CCAGCAAGCTGTGGGCCCAGTGCGTCCAGCTCCACAACGACATCCTGCTGGCCAAGGACACCACAGAGGCCTTCGA |
| | (under- | GAAGATGGTGAGCCTGTTGAGCGTGCTGCTGAGCATGCAGGGCGCCGTGGACATCAACAAGCTGTGCGAGGAAATG |
| | lined) | CTGGACAACCGGGCCACCCTGCAG |
| | NSP7 | |
| 299 | Concatemer | ATGGGGGCCGTGGGCGCCTGCGTGCTGTGTAACAGTCAGACCAGCCTGCGGTGCGGCGCCTGCATCCGCAGGCCC |
| | (NSP13-G4S- | TTCCTGTGCTGTAAGTGCTGCTACGACCACGTGATTAGCACAAGCCACAAGCTCGTGCTCAGCGTGAACCCCTACGT |
| | NSP12-G4S- | GTGCAACGCCCCCGGCTGCGACGTGACAGACGTGACCCAGCTGTACCTGGGCGGAATGAGCTACTATTGTAAGAGT |
| | NSP7) | CACAAGCCCCCTATCAGCTTCCCCCTGTGCGCCAACGGCCAGGTGTTCGGCCTGTACAAGAACACCTGCGTGGGCA |
| | | GCGACAACGTGACCGACTTCAACGCCATCGCCACCTGCGACTGGACCAACGCCGGCGACTACATCCTGGCCAACAC |
| | | CTGCACCGAGCGGCTGAAGCTGTTCGCCGCTGAAACACTGAAGGCCACCGAGGAAACCTTCAAGCTGAGCTACGGC |
| | | ATCGCCACCGTGCGGGAGGTGCTCAGCGATCGGGAACTGCACCTGAGCTGGGAGGTGGGCAAGCCCCGGCCTCCT |
| | | CTGAACCGGAACTACGTGTTCACCGGCTACCGGGTGACCAAGAACAGCAAGGTGCAGATCGGCGAGTACACCTTCG |
| | | AGAAGGGCGACTACGGCGACGCCGTGGTCTACCGGGGCACCACAACCTACAAGCTGAACGTGGGCGACTACTTCGT |
| | | GCTGACCAGCCACACCGTGATGCCCCTGAGCGCCCCCACCCTGGTGCCCCAGGAACACTACGTGCGGATCACCGGC |
| | | CTGTACCCCACCCTGAACATCAGCGACGAGTTCAGCTCTAACGTCGCCAACTACCAGAAAGTGGGAATGCAGAAGTA |
| | | TAGCACACTGCAGGGCCCCCCAGGCACCGGCAAGAGCCACTTCGCCATCGGCCTGGCCCTGTACTACCCCAGCGCC |
| | | CGGATCGTGTACACCGCCTGCAGCCACGCCGCCGTGGACGCCCTGTGCGAGAAGGCCCTGAAGTACCTGCCCATCG |
| | | ACAAGTGCAGCCGGATCATTCCCGCTCGGGCTCGGGTGGAGTGCTTCGACAAGTTCAAGGTGAACAGTACCCTCGA |
| | | GCAGTACGTGTTCTGCACCGTGAACGCCCTGCCCGAAACAACTGCCGACATCGTGGTGTTCGACGAGATCAGCATGG |
| | | CCACCAACTACGACCTGAGCGTGGTTAACGCCCGGCTGCGGGCCAAGCACTACGTGTACATCGGCGACCCCGCCCA |
| | | GCTGCCCGCCCCCCGGACCCTGCTCACCAAGGGCACCCTGGAGCCCGAGTACTTCAACAGCGTGTGCCGGCTGATG |
| | | AAGACCATCGGCCCCGACATGTTCCTGGGCACCTGCCGGCGGTGCCCCGCCGAGATCGTGGACACCGTGAGCGCC |
| | | CTGGTGTACGACAACAAGCTGAAGGCCCACAAGGACAAGAGCGCCCAGTGCTTCAAGATGTTCTACAAGGGCGTGAT |
| | | CACCCACGACGTGAGCAGCGCCATCAACCGGCCCCAGATCGGCGTGGTGCGGGAGTTCCTGACCCGGAACCCCGC |
| | | CTGGCGGAAGGCCGTGTTCATCAGCCCCTACAACAGCCAGAACGCCGTGGCCAGCAAGATCCTGGGCCTGCCCACC |
| | | CAGACCGTGGACAGCTCTCAGGGCAGCGAGTACGACTACGTGATCTTCACCCAGACCACCGAAACAGCCCACAGCT |
| | | GCAACGTCAACCGCTTCAACGTGGCCATCACCCGGGCCAAGGTGGGCATCCTGTGCATCATGAGCGACCGGGACCT |
| | | GTACGACAAGCTGCAGTTCACCAGCCTGGAGATCCCCCGGAGGAACGTGGCCACCCTGCAGGGAGGAGGGGGCTC |
| | | TGGTGGCGGAGGAAGTAGCGCCGACGCCCAGAGCTTCCTGAACCGGGTGTGCGGCGTGAGCGCCGCTCGGCTGAC |
| | | CCCTTGCGGAACCGGAACCAGTACCGACGTGGTCTACCGGGCCTTCGACATCTACAACGACAAGGTGGCCGGCTTC |
| | | GCCAAGTTCCTGAAGACCAACTGCTGTCGGTTCCAGGAGAAGGACGAGGACGATAACCTGATCGACAGCTATTTCGT |
| | | CGTGAAGCGGCACACCTTCAGCAACTACCAGCACGAGGAAACCATCTACAACCTGCTCAAGGACTGCCCCGCCGTG |
| | | GCCAAGCACGACTTCTTTAAGTTCCGGATCGACGGCGACATGGTGCCCCACATCAGTCGGCAACGGCTGACCAAGTA |
| | | CACCATGGCCGACCTCGTGTACGCCCTGCGGCACTTCGACGAGGGCAACTGCGACACCCTGAAGGAGATCCTGGTG |
| | | ACCTACAACTGCTGCGACGACGACTATTTCAATAAGAAAGATTGGTACGATTTTGTGGAAAACCCTGACATCCTGCGG |
| | | GTGTACGCCAACCTGGGCGAGCGGGTGCGGCAGGCCCTCCTGAAAACCGTCCAGTTCTGCGACGCCATGCGGAAC |
| | | GCCGGCATCGTGGGCGTGCTGACCCTGGATAACCAAGACCTGAACGGCAACTGGTACGACTTCGGCGACTTCATCC |
| | | AGACCACACCCGGCAGCGGCGTGCCCGTGGTTGACAGCTACTATAGCCTGTTGATGCCCATCCTGACCCTGACCCG |
| | | GGCCCTGACCGCCGAGAGCCACGTGGATACCGATCTGACAAAGCCCTACATCAAGTGGGACCTGCTGAAGTACGAC |
| | | TTCACCGAGGAGCGGCTGAAGCTGTTCGACCGGTACTTCAAGTACTGGGACCAGACCTACCACCCCAACTGCGTGAA |
| | | CTGCCTGGACGACCGGTGCATCCTGCACTGCGCCAACTTCAACGTGCTGTTCAGCACCGTGTTCCCCCCTACCAGCT |
| | | TCGGCCCCCTGGTGCGGAAGATTTTCGTCGACGGCGTGCCCTTCGTGGTGAGCACCGGCTACCACTTCCGGGAGCT |
| | | GGGCGTGGTGCACAACCAGGACGTGAACCTGCACAGCTCCCGGCTGAGCTTCAAGGAGCTGCTGGTGTACGCCGCC |
| | | GACCCCGCCATGCACGCCGCAAGCGGCAACCTGCTCCTGGACAAGCGGACCACCTGCTTCAGCGTGGCCGCCCTG |
| | | ACCAACAACGTGGCCTTCCAGACAGTGAAACCCGGAAACTTCAACAAGGACTTCTACGACTTCGCCGTGAGCAAGGG |
| | | CTTCTTCAAGGAGGGCAGCAGCGTGGAGCTGAAGCACTTCTTCTTCGCCCAGGACGGCAACGCTGCCATTAGCGACT |
| | | ACGACTACTACCGGTACAACCTGCCCACCATGTGCGACATCCGGCAGCTGTTGTTCGTGGTCGAGGTGGTGGACAAG |
| | | TACTTCGACTGCTACGACGGCGGCTGCATCAACGCCAACCAGGTGATCGTGAACAACCTGGACAAGAGCGCCGGCT |
| | | TCCCCTTCAACAAGTGGGGCAAGGCCCGGCTGTACTACGACAGCATGAGCTACGAGGACCAGGACGCCCTGTTCGC |
| | | CTACACCAAGCGGAACGTGATCCCCACCATCACCCAGATGAACCTGAAGTACGCCATCAGCGCCAAGAACCGGGCC |
| | | CGGACCGTGGCCGGCGTGAGCATCTGCAGCACCATGACCAACCGGCAGTTCCACCAGAAGCTGCTCAAAAGTATCG |
| | | CCGCTACCCGGGGCGCCACCGTGGTTATCGGCACCAGCAAGTTCTACGGCGGCTGGCACAACATGCTGAAGACCGT |
| | | GTACAGCGACGTGGAGAACCCCCACCTGATGGGCTGGGACTACCCCAAGTGCGACCGGGCCATGCCCAACATGCTG |
| | | CGGATCATGGCCAGCCTGGTGCTGGCCCGCAAGCATACCACCTGCTGCAGCCTGAGCCACCGGTTCTACCGGCTGG |
| | | CCAACGAGTGCGCCCAGGTGCTGAGCGAGATGGTGATGTGCGGCGGGAGCCTGTACGTGAAGCCCGGCGGCACCA |
| | | GCTCTGGCGACGCCACCACCGCCTACGCCAACAGCGTGTTCAACATCTGCCAGGCCGTGACCGCCAACGTGAACGC |
| | | CCTGCTCAGCACCGACGGCAACAAGATCGCCGACAAGTACGTGCGGAACCTGCAGCACCGGCTGTACGAGTGCCTG |
| | | TACCGGAACCGGGACGTGGACACCGACTTCGTGAACGAGTTTTACGCCTACCTGCGGAAGCACTTCAGCATGATGAT |
| | | CCTGAGCGACGACGCCGTGGTGTGCTTCAACAGCACCTACGCCAGCCAGGGCCTGGTGGCCAGCATCAAGAACTTC |
| | | AAGAGCGTGCTGTACTACCAGAACAACGTGTTCATGAGCGAGGCCAAGTGCTGGACCGAAACAGACCTGACCAAGG |
| | | GCCCCCACGAGTTCTGCAGCCAGCACACCATGCTGGTGAAGCAGGGCGACGATTACGTGTACCTGCCCTACCCCGA |
| | | CCCCAGCCGGATCCTGGGCGCCGGCTGCTTCGTGGACGACATCGTGAAGACCGACGGCACCCTGATGATCGAGCG |
| | | GTTCGTGAGCCTGGCCATCGACGCCTACCCCCTGACCAAGCACCCCAACCAGGAGTACGCCGACGTGTTCCACCTG |
| | | TACCTGCAGTACATCCGGAAGCTGCACGACGAGCTGACCGGCCACATGCTGGACATGTACAGCGTGATGCTGACCAA |
| | | CGACAACACCAGCCGGTACTGGGAGCCCGAGTTCTACGAGGCCATGTACACCCCCCACACCGTGCTGCAGGGAGGA |
| | | GGGGGCTCTGGTGGCGGAGGAAGTAGCAAGATGAGCGACGTGAAGTGCACCAGCGTGGTCCTGCTCAGCGTGCTG |
| | | CAGCAACTGCGGGTGGAGAGCTCCAGCAAGCTGTGGGCCCAGTGCGTCCAGCTCCACAACGACATCCTGCTGGCCA |
| | | AGGACACCACAGAGGCCTTCGAGAAGATGGTGAGCCTGTTGAGCGTGCTGCTGAGCATGCAGGGCGCCGTGGACAT |
| | | CAACAAGCTGTGCGAGGAAATGCTGGACAACCGGGCCACCCTGCAG |
| 300 | Concatemer | ATGGGGGCCGTGGGCGCCTGCGTGCTGTGTAACAGTCAGACCAGCCTGCGGTGCGGCGCCTGCATCCGCAGGCCC |
| | (NSP13- | TTCCTGTGCTGTAAGTGCTGCTACGACCACGTGATTAGCACAAGCCACAAGCTCGTGCTCAGCGTGAACCCCTACGT |
| | G4furG4S- | GTGCAACGCCCCCGGCTGCGACGTGACAGACGTGACCCAGCTGTACCTGGGCGGAATGAGCTACTATTGTAAGAGT |
| | NSP12- | CACAAGCCCCCTATCAGCTTCCCCCTGTGCGCCAACGGCCAGGTGTTCGGCCTGTACAAGAACACCTGCGTGGGCA |
| | G4furG4S- | GCGACAACGTGACCGACTTCAACGCCATCGCCACCTGCGACTGGACCAACGCCGGCGACTACATCCTGGCCAACAC |
| | NSP7) | CTGCACCGAGCGGCTGAAGCTGTTCGCCGCTGAAACACTGAAGGCCACCGAGGAAACCTTCAAGCTGAGCTACGGC |
| | | ATCGCCACCGTGCGGGAGGTGCTCAGCGATCGGGAACTGCACCTGAGCTGGGAGGTGGGCAAGCCCCGGCCTCCT |
| | | CTGAACCGGAACTACGTGTTCACCGGCTACCGGGTGACCAAGAACAGCAAGGTGCAGATCGGCGAGTACACCTTCG |
| | | AGAAGGGCGACTACGGCGACGCCGTGGTCTACCGGGGCACCACAACCTACAAGCTGAACGTGGGCGACTACTTCGT |
| | | GCTGACCAGCCACACCGTGATGCCCCTGAGCGCCCCCACCCTGGTGCCCCAGGAACACTACGTGCGGATCACCGGC |
| | | CTGTACCCCACCCTGAACATCAGCGACGAGTTCAGCTCTAACGTCGCCAACTACCAGAAAGTGGGAATGCAGAAGTA |
| | | TAGCACACTGCAGGGCCCCCCAGGCACCGGCAAGAGCCACTTCGCCATCGGCCTGGCCCTGTACTACCCCAGCGCC |
| | | CGGATCGTGTACACCGCCTGCAGCCACGCCGCCGTGGACGCCCTGTGCGAGAAGGCCCTGAAGTACCTGCCCATCG |
| | | ACAAGTGCAGCCGGATCATTCCCGCTCGGGCTCGGGTGGAGTGCTTCGACAAGTTCAAGGTGAACAGTACCCTCGA |
| | | GCAGTACGTGTTCTGCACCGTGAACGCCCTGCCCGAAACAACTGCCGACATCGTGGTGTTCGACGAGATCAGCATGG |
| | | CCACCAACTACGACCTGAGCGTGGTTAACGCCCGGCTGCGGGCCAAGCACTACGTGTACATCGGCGACCCCGCCCA |
| | | GCTGCCCGCCCCCCGGACCCTGCTCACCAAGGGCACCCTGGAGCCCGAGTACTTCAACAGCGTGTGCCGGCTGATG |
| | | AAGACCATCGGCCCCGACATGTTCCTGGGCACCTGCCGGCGGTGCCCCGCCGAGATCGTGGACACCGTGAGCGCC |
| | | CTGGTGTACGACAACAAGCTGAAGGCCCACAAGGACAAGAGCGCCCAGTGCTTCAAGATGTTCTACAAGGGCGTGAT |
| | | CACCCACGACGTGAGCAGCGCCATCAACCGGCCCCAGATCGGCGTGGTGCGGGAGTTCCTGACCCGGAACCCCGC |
| | | CTGGCGGAAGGCCGTGTTCATCAGCCCCTACAACAGCCAGAACGCCGTGGCCAGCAAGATCCTGGGCCTGCCCACC |
| | | CAGACCGTGGACAGCTCTCAGGGCAGCGAGTACGACTACGTGATCTTCACCCAGACCACCGAAACAGCCCACAGCT |
| | | GCAACGTCAACCGCTTCAACGTGGCCATCACCCGGGCCAAGGTGGGCATCCTGTGCATCATGAGCGACCGGGACCT |
| | | GTACGACAAGCTGCAGTTCACCAGCCTGGAGATCCCCCGGAGGAACGTGGCCACCCTGCAGGGAGGAGGGGGCAG |
| | | ATCTAAGAGATCTGGTGGCGGAGGAAGTAGCGCCGACGCCCAGAGCTTCCTGAACCGGGTGTGCGGCGTGAGCGC |
| | | CGCTCGGCTGACCCCTTGCGGAACCGGAACCAGTACCGACGTGGTCTACCGGGCCTTCGACATCTACAACGACAAG |
| | | GTGGCCGGCTTCGCCAAGTTCCTGAAGACCAACTGCTGTCGGTTCCAGGAGAAGGACGAGGACGATAACCTGATCG |
| | | ACAGCTATTTCGTCGTGAAGCGGCACACCTTCAGCAACTACCAGCACGAGGAAACCATCTACAACCTGCTCAAGGAC |
| | | TGCCCCGCCGTGGCCAAGCACGACTTCTTTAAGTTCCGGATCGACGGCGACATGGTGCCCCACATCAGTCGGCAAC |
| | | GGCTGACCAAGTACACCATGGCCGACCTCGTGTACGCCCTGCGGCACTTCGACGAGGGCAACTGCGACACCCTGAA |
| | | GGAGATCCTGGTGACCTACAACTGCTGCGACGACGACTATTTCAATAAGAAAGATTGGTACGATTTTGTGGAAAACCC |
| | | TGACATCCTGCGGGTGTACGCCAACCTGGGCGAGCGGGTGCGGCAGGCCCTCCTGAAAACCGTCCAGTTCTGCGAC |
| | | GCCATGCGGAACGCCGGCATCGTGGGCGTGCTGACCCTGGATAACCAAGACCTGAACGGCAACTGGTACGACTTCG |
| | | GCGACTTCATCCAGACCACACCCGGCAGCGGCGTGCCCGTGGTTGACAGCTACTATAGCCTGTTGATGCCCATCCTG |
| | | ACCCTGACCCGGGCCCTGACCGCCGAGAGCCACGTGGATACCGATCTGACAAAGCCCTACATCAAGTGGGACCTGC |
| | | TGAAGTACGACTTCACCGAGGAGCGGCTGAAGCTGTTCGACCGGTACTTCAAGTACTGGGACCAGACCTACCACCCC |
| | | AACTGCGTGAACTGCCTGGACGACCGGTGCATCCTGCACTGCGCCAACTTCAACGTGCTGTTCAGCACCGTGTTCCC |
| | | CCCTACCAGCTTCGGCCCCCTGGTGCGGAAGATTTTCGTCGACGGCGTGCCCTTCGTGGTGAGCACCGGCTACCAC |
| | | TTCCGGGAGCTGGGCGTGGTGCACAACCAGGACGTGAACCTGCACAGCTCCCGGCTGAGCTTCAAGGAGCTGCTGG |
| | | TGTACGCCGCCGACCCCGCCATGCACGCCGCAAGCGGCAACCTGCTCCTGGACAAGCGGACCACCTGCTTCAGCGT |
| | | GGCCGCCCTGACCAACAACGTGGCCTTCCAGACAGTGAAACCCGGAAACTTCAACAAGGACTTCTACGACTTCGCCG |
| | | TGAGCAAGGGCTTCTTCAAGGAGGGCAGCAGCGTGGAGCTGAAGCACTTCTTCTTCGCCCAGGACGGCAACGCTGC |
| | | CATTAGCGACTACGACTACTACCGGTACAACCTGCCCACCATGTGCGACATCCGGCAGCTGTTGTTCGTGGTCGAGG |
| | | TGGTGGACAAGTACTTCGACTGCTACGACGGCGGCTGCATCAACGCCAACCAGGTGATCGTGAACAACCTGGACAAG |
| | | AGCGCCGGCTTCCCCTTCAACAAGTGGGGCAAGGCCCGGCTGTACTACGACAGCATGAGCTACGAGGACCAGGACG |
| | | CCCTGTTCGCCTACACCAAGCGGAACGTGATCCCCACCATCACCCAGATGAACCTGAAGTACGCCATCAGCGCCAAG |
| | | AACCGGGCCCGGACCGTGGCCGGCGTGAGCATCTGCAGCACCATGACCAACCGGCAGTTCCACCAGAAGCTGCTCA |
| | | AAAGTATCGCCGCTACCCGGGGCGCCACCGTGGTTATCGGCACCAGCAAGTTCTACGGCGGCTGGCACAACATGCT |
| | | GAAGACCGTGTACAGCGACGTGGAGAACCCCCACCTGATGGGCTGGGACTACCCCAAGTGCGACCGGGCCATGCC |
| | | CAACATGCTGCGGATCATGGCCAGCCTGGTGCTGGCCCGCAAGCATACCACCTGCTGCAGCCTGAGCCACCGGTTC |
| | | TACCGGCTGGCCAACGAGTGCGCCCAGGTGCTGAGCGAGATGGTGATGTGCGGGGGGAGCCTGTACGTGAAGCCC |
| | | GGCGGCACCAGCTCTGGCGACGCCACCACCGCCTACGCCAACAGCGTGTTCAACATCTGCCAGGCCGTGACCGCCA |
| | | ACGTGAACGCCCTGCTCAGCACCGACGGCAACAAGATCGCCGACAAGTACGTGCGGAACCTGCAGCACCGGCTGTA |
| | | CGAGTGCCTGTACCGGAACCGGGACGTGGACACCGACTTCGTGAACGAGTTTTACGCCTACCTGCGGAAGCACTTCA |
| | | GCATGATGATCCTGAGCGACGACGCCGTGGTGTGCTTCAACAGCACCTACGCCAGCCAGGGCCTGGTGGCCAGCAT |
| | | CAAGAACTTCAAGAGCGTGCTGTACTACCAGAACAACGTGTTCATGAGCGAGGCCAAGTGCTGGACCGAAACAGACC |
| | | TGACCAAGGGCCCCCACGAGTTCTGCAGCCAGCACACCATGCTGGTGAAGCAGGGCGACGATTACGTGTACCTGCC |
| | | CTACCCCGACCCCAGCCGGATCCTGGGCGCCGGCTGCTTCGTGGACGACATCGTGAAGACCGACGGCACCCTGATG |
| | | ATCGAGCGGTTCGTGAGCCTGGCCATCGACGCCTACCCCCTGACCAAGCACCCCAACCAGGAGTACGCCGACGTGT |
| | | TCCACCTGTACCTGCAGTACATCCGGAAGCTGCACGACGAGCTGACCGGCCACATGCTGGACATGTACAGCGTGATG |
| | | CTGACCAACGACAACACCAGCCGGTACTGGGAGCCCGAGTTCTACGAGGCCATGTACACCCCCCACACCGTGCTGC |
| | | AGGGAGGAGGGGGCAGATCTAAGAGATCTGGTGGCGGAGGAAGTAGCAAGATGAGCGACGTGAAGTGCACCAGCG |
| | | TGGTCCTGCTCAGCGTGCTGCAGCAACTGCGGGTGGAGAGCTCCAGCAAGCTGTGGGCCCAGTGCGTCCAGCTCCA |
| | | CAACGACATCCTGCTGGCCAAGGACACCACAGAGGCCTTCGAGAAGATGGTGAGCCTGTTGAGCGTGCTGCTGAGC |
| | | ATGCAGGGCGCCGTGGACATCAACAAGCTGTGCGAGGAAATGCTGGACAACCGGGCCACCCTGCAG |
| 301 | SARS-CoV- | MGVKVLFALICIAVAEAKRVQPTESIVRFPNITNLCPFGEVFNATRFASVYAWNRKRISNCVADYSVLYNSASFSTFKCYGVS |
| | 2 RBD | PTKLNDLCFTNVYADSFVIRGDEVRQIAPGQTGKIADYNYKLPDDFTGCVIAWNSNNLDSKVGGNYNYLYRLFRKSNLKPFE |
| | immunogen | RDISTEIYQAGSTPCNGVEGFNCYFPLQSYGFQPTNGVGYQPYRVVVLSFELLHAPATVCGPKKSTNLVKNKCVNFGYIPE |
| | fused to | APRDGQAYVRKDGEWVLLSTFL |
| | a T4 foldon | |
| | multi- | |
| | merization | |
| | domain | |
| 302 | Spike | MFVFLVLLPLVSSQCVNLTTRTQLPPAYTNSFTRGVYYPDKVFRSSVLHSTQDLFLPFFSNVTWFHAIHVSGTNGTKRFDN |
| | | PVLPFNDGVYFASTEKSNIIRGWIFGTTLDSKTQSLLIVNNATNVVIKVCEFQFCNDPFLGVYYHKNNKSWMESEFRVYSSA |
| | | NNCTFEYVSQPFLMDLEGKQGNFKNLREFVFKNIDGYFKIYSKHTPINLVRDLPQGFSALEPLVDLPIGINITRFQTLLALHRS |
| | | YLTPGDSSSGWTAGAAAYYVGYLQPRTFLLKYNENGTITDAVDCALDPLSETKCTLKSFTVEKGIYQTSNFRVQPTESIVRF |
| | | PNITNLCPFGEVFNATRFASVYAWNRKRISNCVADYSVLYNSASFSTFKCYGVSPTKLNDLCFTNVYADSFVIRGDEVRQIA |
| | | PGQTGKIADYNYKLPDDFTGCVIAWNSNNLDSKVGGNYNYLYRLFRKSNLKPFERDISTEIYQAGSTPCNGVEGFNCYFPL |
| | | QSYGFQPTNGVGYQPYRVVVLSFELLHAPATVCGPKKSTNLVKNKCVNFNFNGLTGTGVLTESNKKFLPFQQFGRDIADTT |
| | | DAVRDPQTLEILDITPCSFGGVSVITPGTNTSNQVAVLYQDVNCTEVPVAIHADQLTPTWRVYSTGSNVFQTRAGCLIGAEH |
| | | VNNSYECDIPIGAGICASYQTQTNSPRRARSVASQSIIAYTMSLGAENSVAYSNNSIAIPTNFTISVTTEILPVSMTKTSVDCT |
| | | MYICGDSTECSNLLLQYGSFCTQLNRALTGIAVEQDKNTQEVFAQVKQIYKTPPIKDFGGFNFSQILPDPSKPSKRSFIEDLL |
| | | FNKVTLADAGFIKQYGDCLGDIAARDLICAQKFNGLTVLPPLLTDEMIAQYTSALLAGTITSGWTFGAGAALQIPFAMQMAYR |
| | | FNGIGVTQNVLYENQKLIANQFNSAIGKIQDSLSSTASALGKLQDVVNQNAQALNTLVKQLSSNFGAISSVLNDILSRLDPPE |
| | | AEVQIDRLITGRLQSLQTYVTQQLIRAAEIRASANLAATKMSECVLGQSKRVDFCGKGYHLMSFPQSAPHGVVFLHVTYVPA |
| | | QEKNFTTAPAICHDGKAHFPREGVFVSNGTHWFVTQRNFYEPQIITTDNTFVSGNCDVVIGIVNNTVYDPLQPELDSFKEEL |
| | | DKYFKNHTSPDVDLGDISGINASVVNIQKEIDRLNEVAKNLNESLIDLQELGKYEQYIKWPWYIWLGFIAGLIAIVMVTIMLCC |
| | | MTSCCSCLKGCCSCGSCCKFDEDDSEPVLKGVKLHYT |
| 303 | SARS-CoV- | ATGGGAGTCAAAGTTCTGTTTGCCCTGATCTGCATTGCTGTGGCCGAGGCCAAGAGAGTCCAACCAACAGAATCTATT |
| | 2 RBD | GTTAGATTTCCTAATATTACAAACTTGTGCCCTTTTGGTGAAGTTTTTAACGCCACCAGATTTGCATCCGTGTATGCTTG |
| | immunogen | GAACAGGAAGAGAATCAGCAACTGTGTTGCTGATTATTCTGTCCTATATAATTCCGCATCATTTTCCACTTTTAAGTGTT |
| | fused to | ATGGAGTGTCTCCTACTAAATTAAATGATCTCTGCTTTACTAATGTCTATGCAGATTCATTTGTAATTAGAGGTGATGAA |
| | a T4 foldon | GTCAGACAAATCGCTCCAGGGCAAACTGGAAAGATTGCTGATTATAATTATAAATTACCAGATGATTTTACAGGCTGCG |
| | multi- | TTATAGCTTGGAATTCTAACAATCTTGATTCTAAGGTTGGTGGTAATTATAATTACCTGTATAGATTGTTTAGGAAGTCT |
| | merization | AATCTCAAACCTTTTGAGAGAGATATTTCAACTGAAATCTATCAGGCCGGTAGCACACCTTGTAATGGTGTTGAAGGTT |
| | domain | TTAATTGTTACTTTCCTTTACAATCATATGGTTTCCAACCCACTAATGGTGTTGGTTACCAACCATACAGAGTAGTAGTA |
| | | CTTTCTTTTGAACTTCTACATGCACCAGCAACTGTTTGTGGACCTAAAAAGTCTACTAATTTGGTTAAAAACAAATGTGT |
| | | CAATTTCGGGTATATCCCTGAAGCCCCCAGGGACGGCCAGGCTTACGTCAGAAAGGATGGAGAGTGGGTGCTCTTGA |
| | | GCACCTTCCTG |
| 304 | Spike | ATGTTCGTGTTCCTGGTGCTGCTGCCTCTGGTGTCCAGCCAGTGTGTGAACCTGACCACCAGAACACAGCTGCCTCC |
| | | AGCCTACACCAACAGCTTTACCAGAGGCGTGTACTACCCCGACAAGGTGTTCAGATCCAGCGTGCTGCACTCTACCC |
| | | AGGACCTGTTCCTGCCTTTCTTCAGCAACGTGACCTGGTTCCACGCCATCCACGTGTCCGGCACCAATGGCACCAAG |
| | | AGATTCGACAACCCCGTGCTGCCCTTCAACGACGGGGTGTACTTTGCCAGCACCGAGAAGTCCAACATCATCAGAGG |
| | | CTGGATCTTCGGCACCACACTGGACAGCAAGACCCAGAGCCTGCTGATCGTGAACAACGCCACCAACGTGGTCATCA |
| | | AAGTGTGCGAGTTCCAGTTCTGCAACGACCCCTTCCTGGGCGTCTACTACCACAAGAACAACAAGAGCTGGATGGAA |
| | | AGCGAGTTCCGGGTGTACAGCAGCGCCAACAACTGCACCTTCGAGTACGTGTCCCAGCCTTTCCTGATGGACCTGGA |
| | | AGGCAAGCAGGGCAACTTCAAGAACCTGCGCGAGTTCGTGTTTAAGAACATCGACGGCTACTTCAAGATCTACAGCA |
| | | AGCACACCCCTATCAACCTCGTGCGGGATCTGCCTCAGGGCTTCTCTGCTCTGGAACCCCTGGTGGATCTGCCCATC |
| | | GGCATCAACATCACCCGGTTTCAGACACTGCTGGCCCTGCACAGAAGCTACCTGACACCTGGCGATAGCAGCAGCG |
| | | GATGGACAGCTGGTGCCGCCGCTTACTATGTGGGCTACCTGCAGCCTAGAACCTTCCTGCTGAAGTACAACGAGAAC |
| | | GGCACCATCACCGACGCCGTGGATTGTGCTCTGGATCCTCTGAGCGAGACAAAGTGCACCCTGAAGTCCTTCACCGT |
| | | GGAAAAGGGCATCTACCAGACCAGCAACTTCCGGGTGCAGCCCACCGAATCCATCGTGCGGTTCCCCAATATCACCA |
| | | ATCTGTGCCCCTTCGGCGAGGTGTTCAATGCCACCAGATTCGCCTCTGTGTACGCCTGGAACCGGAAGCGGATCAGC |
| | | AATTGCGTGGCCGACTACTCCGTGCTGTACAACTCCGCCAGCTTCAGCACCTTCAAGTGCTACGGCGTGTCCCCTAC |
| | | CAAGCTGAACGACCTGTGCTTCACAAACGTGTACGCCGACAGCTTCGTGATCCGGGGAGATGAAGTGCGGCAGATTG |
| | | CCCCTGGACAGACAGGCAAGATCGCCGACTACAACTACAAGCTGCCCGACGACTTCACCGGCTGTGTGATTGCCTG |
| | | GAACAGCAACAACCTGGACTCCAAAGTCGGCGGCAACTACAATTACCTGTACCGGCTGTTCCGGAAGTCCAATCTGA |
| | | AGCCCTTCGAGCGGGACATCTCCACCGAGATCTATCAGGCCGGCAGCACCCCTTGTAACGGCGTGGAAGGCTTCAA |
| | | CTGCTACTTCCCACTGCAGTCCTACGGCTTTCAGCCCACAAATGGCGTGGGCTATCAGCCCTACAGAGTGGTGGTGC |
| | | TGAGCTTCGAACTGCTGCATGCCCCTGCCACAGTGTGCGGCCCTAAGAAAAGCACCAATCTCGTGAAGAACAAATGC |
| | | GTGAACTTCAACTTCAACGGCCTGACCGGCACCGGCGTGCTGACAGAGAGCAACAAGAAGTTCCTGCCATTCCAGCA |
| | | GTTTGGCCGGGATATCGCCGATACCACAGACGCCGTTAGAGATCCCCAGACACTGGAAATCCTGGACATCACCCCTT |
| | | GCAGCTTCGGCGGAGTGTCTGTGATCACCCCTGGCACCAACACCAGCAATCAGGTGGCAGTGCTGTACCAGGACGT |
| | | GAACTGTACCGAAGTGCCCGTGGCCATTCACGCCGATCAGCTGACACCTACATGGCGGGTGTACTCCACCGGCAGC |
| | | AATGTGTTTCAGACCAGAGCCGGCTGTCTGATCGGAGCCGAGCACGTGAACAATAGCTACGAGTGCGACATCCCCAT |
| | | CGGCGCTGGAATCTGCGCCAGCTACCAGACACAGACAAACAGCCCTCGGAGAGCCAGAAGCGTGGCCAGCCAGAG |
| | | CATCATTGCCTACACAATGTCTCTGGGCGCCGAGAACAGCGTGGCCTACTCCAACAACTCTATCGCTATCCCCACCAA |
| | | CTTCACCATCAGCGTGACCACAGAGATCCTGCCTGTGTCCATGACCAAGACCAGCGTGGACTGCACCATGTACATCT |
| | | GCGGCGATTCCACCGAGTGCTCCAACCTGCTGCTGCAGTACGGCAGCTTCTGCACCCAGCTGAATAGAGCCCTGAC |
| | | AGGGATCGCCGTGGAACAGGACAAGAACACCCAAGAGGTGTTCGCCCAAGTGAAGCAGATCTACAAGACCCCTCCTA |
| | | TCAAGGACTTCGGCGGCTTCAATTTCAGCCAGATTCTGCCCGATCCTAGCAAGCCCAGCAAGCGGAGCTTCATCGAG |
| | | GACCTGCTGTTCAACAAAGTGACACTGGCCGACGCCGGCTTCATCAAGCAGTATGGCGATTGTCTGGGCGACATTGC |
| | | CGCCAGGGATCTGATTTGCGCCCAGAAGTTTAACGGACTGACAGTGCTGCCTCCTCTGCTGACCGATGAGATGATCG |
| | | CCCAGTACACATCTGCCCTGCTGGCCGGCACAATCACAAGCGGCTGGACATTTGGAGCAGGCGCCGCTCTGCAGAT |
| | | CCCCTTTGCTATGCAGATGGCCTACCGGTTCAACGGCATCGGAGTGACCCAGAATGTGCTGTACGAGAACCAGAAGC |
| | | TGATCGCCAACCAGTTCAACAGCGCCATCGGCAAGATCCAGGACAGCCTGAGCAGCACAGCAAGCGCCCTGGGAAA |
| | | GCTGCAGGACGTGGTCAACCAGAATGCCCAGGCACTGAACACCCTGGTCAAGCAGCTGTCCTCCAACTTCGGCGCC |
| | | ATCAGCTCTGTGCTGAACGATATCCTGAGCAGACTGGACCCTCCTGAGGCCGAGGTGCAGATCGACAGACTGATCAC |
| | | AGGCAGACTGCAGAGCCTCCAGACATACGTGACCCAGCAGCTGATCAGAGCCGCCGAGATTAGAGCCTCTGCCAAT |
| | | CTGGCCGCCACCAAGATGTCTGAGTGTGTGCTGGGCCAGAGCAAGAGAGTGGACTTTTGCGGCAAGGGCTACCACC |
| | | TGATGAGCTTCCCTCAGTCTGCCCCTCACGGCGTGGTGTTTCTGCACGTGACATATGTGCCCGCTCAAGAGAAGAAT |
| | | TTCACCACCGCTCCAGCCATCTGCCACGACGGCAAAGCCCACTTTCCTAGAGAAGGCGTGTTCGTGTCCAACGGCAC |
| | | CCATTGGTTCGTGACACAGCGGAACTTCTACGAGCCCCAGATCATCACCACCGACAACACCTTCGTGTCTGGCAACT |
| | | GCGACGTCGTGATCGGCATTGTGAACAATACCGTGTACGACCCTCTGCAGCCCGAGCTGGACAGCTTCAAAGAGGAA |
| | | CTGGACAAGTACTTTAAGAACCACACAAGCCCCGACGTGGACCTGGGCGATATCAGCGGAATCAATGCCAGCGTCGT |
| | | GAACATCCAGAAAGAGATCGACCGGCTGAACGAGGTGGCCAAGAATCTGAACGAGAGCCTGATCGACCTGCAAGAA |
| | | CTGGGGAAGTACGAGCAGTACATCAAGTGGCCCTGGTACATCTGGCTGGGCTTTATCGCCGGACTGATTGCCATCGT |
| | | GATGGTCACAATCATGCTGTGTTGCATGACCAGCTGCTGTAGCTGCCTGAAGGGCTGTTGTAGCTGTGGCAGCTGCT |
| | | GCAAGTTCGACGAGGACGATTCTGAGCCCGTGCTGAAGGGCGTGAAACTGCACTACACA |
| 305 | CVB3 | TTAAAACAGCCTGTGGGTTGATCCCACCCACAGGCCCATTGGGCGCTAGCACTCTGGTATCACGGTACCTTTGTGCG |
| | modified | CCTGTTTTATACCCCCTCCCCCAACTGTAACTTAGAAGTAACACACACCGATCAACAGTCAGCGTGGCACACCAGCCA |
| | | CGTTTTGATCAAGCACTTCTGTTACCCCGGACTGAGTATCAATAGACTGCTCACGCGGTTGAAGGAGAAAGCGTTCGT |
| | | TATCCGGCCAACTACTTCGAAAAACCTAGTAACACCGTGGAAGTTGCAGAGTGTTTCGCTCAGCACTACCCCAGTGTA |
| | | GATCAGGTCGATGAGTCACCGCATTCCCCACGGGCGACCGTGGCGGTGGCTGCGTTGGCGGCCTGCCCATGGGGA |
| | | AACCCATGGGACGCTCTAATACAGACATGGTGCGAAGAGTCTATTGAGCTAGTTGGTAGTCCTCCGGCCCCTGAATG |
| | | CGGCTAATCCTAACTGCGGAGCACACACCCTCAAGCCAGAGGGCAGTGTGTCGTAACGGGCAACTCTGCAGCGGAA |
| | | CCGACTACTTTGGGTGTCCGTGTTTCATTTTATTCCTATACTGGCTGCTTATGGTGACAATTGAGAGATCGTTACCATA |
| | | TAGCTATTGGATTGGCCATCCGGTGACTAATAGAGCTATTATATATCCCTTTGTTGGGTTTATACCACTTAGCTTGAAA |
| | | GAGGTTAAAACATTACAATTCATTGTTAAGTTGAATACAGCAAC |
| 306 | EV71 | TTAAAACAGCTGTGGGTTGTCACCCACCCACAGGGTCCACTGGGCGCTAGTACACTGGTATCTCGGTACCTTTGTAC |
| | modified | GCCTGTTTTATACCCCCTCCCTGATTTGCAACTTAGAAGCAACGCAAACCAGATCAATAGTAGGTGTGACATACCAGT |
| | | CGCATCTTGATCAAGCACTTCTGTATCCCCGGACCGAGTATCAATAGACTGTGCACACGGTTGAAGGAGAAAACGTCC |
| | | GTTACCCGGCTAACTACTTCGAGAAGCCTAGTAACGCCATTGAAGTTGCAGAGTGTTTCGCTCAGCACTCCCCCCGT |
| | | GTAGATCAGGTCGATGAGTCACCGCATTCCCCACGGGCGACCGTGGCGGTGGCTGCGTTGGCGGCCTGCCTATGG |
| | | GGTAACCCATAGGACGCTCTAATACGGACATGGCGTGAAGAGTCTATTGAGCTAGTTAGTAGTCCTCCGGCCCCTGA |
| | | ATGCGGCTAATCCTAACTGCGGAGCACATACCCTTAATCCAAAGGGCAGTGTGTCGTAACGGGCAACTCTGCAGCGG |
| | | AACCGACTACTTTGGGTGTCCGTGTTTCTTTTTATTCTTGTATTGGCTGCTTATGGTGACAATTAAAGAATTGTTACCAT |
| | | ATAGCTATTGGATTGGCCATCCAGTGTCAAACAGAGCTATTGTATATCTCTTTGTTGGATTCACACCTCTCACTCTTGA |
| | | AACGTTACACACCCTCAATTACATTATACTGCTGAACACGAAGCGGCCACC |

1. A circular polyribonucleotide comprising an open reading frame encoding a coronavirus immunogen, wherein the coronavirus immunogen comprises an amino acid sequence having at least 95% sequence identity with the amino acid sequence of any one of SEQ ID NOs: 63-111 and 283-291, wherein the circular polyribonucleotide further comprises a sequence encoding an influenza immunogen.

2. The circular polyribonucleotide of claim 1, wherein the coronavirus immunogen is a RBD immunogen having at least 95% identity with the amino acid sequence of any one of SEQ ID NOs: 63-68, 74, 79, 81-86, and 98-111.

3. The circular polyribonucleotide of claim 1, wherein the coronavirus immunogen is a Spike immunogen having at least 95% identity with the amino acid sequence of any one of SEQ ID NOs: 69-73, 75-78, 80, 87-97 and 283-286.

4. The circular polyribonucleotide of claim 1, wherein the coronavirus immunogen is a nonstructural protein (nsp) having at least 95% identity with the amino acid sequence of any one of SEQ ID NOs: 291-295.

5. (canceled)

6. The circular polyribonucleotide of claim 1, wherein the open reading frame comprises a nucleic acid sequence having at least 95% sequence identity with the nucleic acid sequence of any one of SEQ ID NOs: 112-174 and 292-300.

7. The circular polyribonucleotide of claim 6, wherein the coronavirus immunogen is a RBD immunogen having at least 95% identity with the nucleic acid sequence of any one of SEQ ID NOs: 112-117, 123, 128, 133-138, and 163-174.

8. The circular polyribonucleotide of claim 6, wherein the coronavirus immunogen is a Spike immunogen having at least 95% identity with the nucleic sequence of any one of SEQ ID NOs: 118-122, 124-127, 129-132, 139-162, and 287-291.

9. The circular polyribonucleotide of claim 6, wherein the coronavirus immunogen is a nsp having at least 95% identity with the nucleic sequence of any one of SEQ ID NOs: 296-300.

10-15. (canceled)

16. A circular polyribonucleotide comprising a first sequence encoding a coronavirus immunogen having at least 95% sequence identity with the amino acid sequence of any one of SEQ ID NOs: 63-111 and 283-291 and a second sequence encoding a polypeptide adjuvant and/or multimerization domain.

17. (canceled)

18. An immunogenic composition comprising a first circular polyribonucleotide comprising an open reading frame encoding a coronavirus immunogen, wherein the coronavirus immunogen comprises an amino acid sequence having at least 95% sequence identity with the amino acid sequence of any one of SEQ ID NOs: 63-111 and 283-291, a second circular polyribonucleotide, and a pharmaceutically acceptable carrier or excipient.

19. (canceled)

20. The immunogenic composition of claim 18, wherein the second circular polyribonucleotide comprises an open reading frame encoding a second polypeptide immunogen.

21. The immunogenic composition of claim 18, wherein the second circular polyribonucleotide comprises an open reading frame encoding a polypeptide adjuvant.

22. A linear polyribonucleotide comprising an open reading frame encoding a coronavirus immunogen, wherein the coronavirus immunogen comprises an amino acid sequence having at least 95% sequence identity with the amino acid sequence of any one of SEQ ID NOs: 63-111 and 283-291, wherein the linear polyribonucleotide further comprises a sequence encoding an influenza immunogen.

23. (canceled)

24. The linear polyribonucleotide of claim 22, wherein the open reading frame comprises a nucleic acid sequence having at least 95% sequence identity with the nucleic acid sequence of any one of SEQ ID NOs: 112-174 and 292-300.

25-29. (canceled)

30. A linear polyribonucleotide comprising a first sequence encoding a coronavirus immunogen having at least 95% sequence identity with the amino acid sequence of any one of SEQ ID NOs: 63-111 and 283-291 and a second sequence encoding a multimerization domain, a second coronavirus immunogen, and/or polypeptide adjuvant.

31. An immunogenic composition comprising the linear polyribonucleotide of claim 22 and a pharmaceutically acceptable carrier or excipient.

32. The immunogenic composition of claim 31, wherein the composition further comprises a second linear polyribonucleotide, wherein the second linear polyribonucleotide comprises an open reading frame encoding a second polypeptide immunogen and/or polypeptide adjuvant.

33-34. (canceled)

35. A method of inducing an immune response in a subject against SARS-CoV-2, the method administering to the subject the circular polyribonucleotide of claim 1.

36. A method of preventing a SARS-CoV-2 infection in a subject, the method comprising administering to the subject the circular polyribonucleotide of claim 1.
